SEQUENCE LISTING (793 sequences)

SEQ ID NO: 1 - DNA, 52 bp, synthetic construct
  source 1..52: mol_type = other DNA
tcgtcggcagcgtcagatgtgtataagagacaggtgycagcmgccgcggtaa

SEQ ID NO: 2 - DNA, 54 bp, synthetic construct
  source 1..54: mol_type = other DNA
taatctwtgggvncatcagggacagagaatatgtgtagaggctcgggtgctctg

SEQ ID NO: 3 - DNA, 13 bp, synthetic construct
  source 1..13: mol_type = other DNA; note = RT Primer
aagtgattagcaa

SEQ ID NO: 4 - DNA, 60 bp, synthetic construct
  source 1..60: mol_type = other DNA; note = RT Primer
tcgtcggcagcgtcagatgtgtataagagacagnnnnnnnnaagtgattagcaannnnnn

SEQ ID NO: 5 - DNA, 13 bp, synthetic construct
  source 1..13: mol_type = other DNA; note = RT Primer
agaatccccctaa

SEQ ID NO: 6 - DNA, 60 bp, synthetic construct
  source 1..60: mol_type = other DNA; note = RT Primer
tcgtcggcagcgtcagatgtgtataagagacagnnnnnnnnagaatccccctaannnnnn

SEQ ID NO: 7 - DNA, 13 bp, synthetic construct
  source 1..13: mol_type = other DNA; note = RT Primer
acctgggaaacta

SEQ ID NO: 8 - DNA, 60 bp, synthetic construct
  source 1..60: mol_type = other DNA; note = RT Primer
tcgtcggcagcgtcagatgtgtataagagacagnnnnnnnnacctgggaaactannnnnn

SEQ ID NO: 9 - DNA, 13 bp, synthetic construct
  source 1..13: mol_type = other DNA; note = RT Primer
atacctcccagga

SEQ ID NO: 10 - DNA, 60 bp, synthetic construct
  source 1..60: mol_type = other DNA; note = RT Primer
tcgtcggcagcgtcagatgtgtataagagacagnnnnnnnnatacctcccaggannnnnn

SEQ ID NO: 11 - DNA, 13 bp, synthetic construct
  source 1..13: mol_type = other DNA; note = RT Primer
aatttgtggtata

SEQ ID NO: 12 - DNA, 60 bp, synthetic construct
  source 1..60: mol_type = other DNA; note = RT Primer
tcgtcggcagcgtcagatgtgtataagagacagnnnnnnnnaatttgtggtatannnnnn

SEQ ID NO: 13 - DNA, 13 bp, synthetic construct
  source 1..13: mol_type = other DNA; note = RT Primer
acccgagagatca

SEQ ID NO: 14 - DNA, 60 bp, synthetic construct
  source 1..60: mol_type = other DNA; note = RT Primer
tcgtcggcagcgtcagatgtgtataagagacagnnnnnnnnacccgagagatcannnnnn

SEQ ID NO: 15 - DNA, 13 bp, synthetic construct
  source 1..13: mol_type = other DNA; note = RT Primer
agagtatagggta

SEQ ID NO: 16 - DNA, 60 bp, synthetic construct
  source 1..60: mol_type = other DNA; note = RT Primer
tcgtcggcagcgtcagatgtgtataagagacagnnnnnnnnagagtatagggtannnnnn

SEQ ID NO: 17 - DNA, 13 bp, synthetic construct
  source 1..13: mol_type = other DNA; note = RT Primer
atcttaattgaga

SEQ ID NO: 18 - DNA, 60 bp, synthetic construct
  source 1..60: mol_type = other DNA; note = RT Primer
tcgtcggcagcgtcagatgtgtataagagacagnnnnnnnnatcttaattgagannnnnn

SEQ ID NO: 19 - DNA, 13 bp, synthetic construct
  source 1..13: mol_type = other DNA; note = RT Primer
aacgttctgtcga

SEQ ID NO: 20 - DNA, 60 bp, synthetic construct
  source 1..60: mol_type = other DNA; note = RT Primer
tcgtcggcagcgtcagatgtgtataagagacagnnnnnnnnaacgttctgtcgannnnnn

SEQ ID NO: 21 - DNA, 13 bp, synthetic construct
  source 1..13: mol_type = other DNA; note = RT Primer
acataatggcgaa

SEQ ID NO: 22 - DNA, 60 bp, synthetic construct
  source 1..60: mol_type = other DNA; note = RT Primer
tcgtcggcagcgtcagatgtgtataagagacagnnnnnnnnacataatggcgaannnnnn

SEQ ID NO: 23 - DNA, 13 bp, synthetic construct
  source 1..13: mol_type = other DNA; note = RT Primer
aggccggaacata

SEQ ID NO: 24 - DNA, 60 bp, synthetic construct
  source 1..60: mol_type = other DNA; note = RT Primer
tcgtcggcagcgtcagatgtgtataagagacagnnnnnnnnaggccggaacatannnnnn

SEQ ID NO: 25 - DNA, 13 bp, synthetic construct
  source 1..13: mol_type = other DNA; note = RT Primer
atcacttcaagaa

SEQ ID NO: 26 - DNA, 60 bp, synthetic construct
  source 1..60: mol_type = other DNA; note = RT Primer
tcgtcggcagcgtcagatgtgtataagagacagnnnnnnnnatcacttcaagaannnnnn

SEQ ID NO: 27 - DNA, 13 bp, synthetic construct
  source 1..13: mol_type = other DNA; note = RT Primer
aagctggccttca

SEQ ID NO: 28 - DNA, 60 bp, synthetic construct
  source 1..60: mol_type = other DNA; note = RT Primer
tcgtcggcagcgtcagatgtgtataagagacagnnnnnnnnaagctggccttcannnnnn

SEQ ID NO: 29 - DNA, 13 bp, synthetic construct
  source 1..13: mol_type = other DNA; note = RT Primer
actgcacattgga

SEQ ID NO: 30 - DNA, 60 bp, synthetic construct
  source 1..60: mol_type = other DNA; note = RT Primer
tcgtcggcagcgtcagatgtgtataagagacagnnnnnnnnactgcacattggannnnnn

SEQ ID NO: 31 - DNA, 13 bp, synthetic construct
  source 1..13: mol_type = other DNA; note = RT Primer
aggtccttcgata

SEQ ID NO: 32 - DNA, 60 bp, synthetic construct
  source 1..60: mol_type = other DNA; note = RT Primer
tcgtcggcagcgtcagatgtgtataagagacagnnnnnnnnaggtccttcgatannnnnn

SEQ ID NO: 33 - DNA, 13 bp, synthetic construct
  source 1..13: mol_type = other DNA; note = RT Primer
attcgcacccgaa

SEQ ID NO: 34 - DNA, 60 bp, synthetic construct
  source 1..60: mol_type = other DNA; note = RT Primer
tcgtcggcagcgtcagatgtgtataagagacagnnnnnnnnattcgcacccgaannnnnn

SEQ ID NO: 35 - DNA, 13 bp, synthetic construct
  source 1..13: mol_type = other DNA; note = RT Primer
accgtaaattaca

SEQ ID NO: 36 - DNA, 60 bp, synthetic construct
  source 1..60: mol_type = other DNA; note = RT Primer
tcgtcggcagcgtcagatgtgtataagagacagnnnnnnnnaccgtaaattacannnnnn

SEQ ID NO: 37 - DNA, 13 bp, synthetic construct
  source 1..13: mol_type = other DNA; note = RT Primer
agtagtcgatata

SEQ ID NO: 38 - DNA, 60 bp, synthetic construct
  source 1..60: mol_type = other DNA; note = RT Primer
tcgtcggcagcgtcagatgtgtataagagacagnnnnnnnnagtagtcgatatannnnnn

SEQ ID NO: 39 - DNA, 13 bp, synthetic construct
  source 1..13: mol_type = other DNA; note = RT Primer
atgtaagaatcaa

SEQ ID NO: 40 - DNA, 60 bp, synthetic construct
  source 1..60: mol_type = other DNA; note = RT Primer
tcgtcggcagcgtcagatgtgtataagagacagnnnnnnnnatgtaagaatcaannnnnn

SEQ ID NO: 41 - DNA, 13 bp, synthetic construct
  source 1..13: mol_type = other DNA; note = RT Primer
aaggcccgccgta

SEQ ID NO: 42 - DNA, 60 bp, synthetic construct
  source 1..60: mol_type = other DNA; note = RT Primer
tcgtcggcagcgtcagatgtgtataagagacagnnnnnnnnaaggcccgccgtannnnnn

SEQ ID NO: 43 - DNA, 13 bp, synthetic construct
  source 1..13: mol_type = other DNA; note = RT Primer
actccacgcaaga

SEQ ID NO: 44 - DNA, 60 bp, synthetic construct
  source 1..60: mol_type = other DNA; note = RT Primer
tcgtcggcagcgtcagatgtgtataagagacagnnnnnnnnactccacgcaagannnnnn

SEQ ID NO: 45 - DNA, 13 bp, synthetic construct
  source 1..13: mol_type = other DNA; note = RT Primer
agtctttgattaa

SEQ ID NO: 46 - DNA, 60 bp, synthetic construct
  source 1..60: mol_type = other DNA; note = RT Primer
tcgtcggcagcgtcagatgtgtataagagacagnnnnnnnnagtctttgattaannnnnn

SEQ ID NO: 47 - DNA, 13 bp, synthetic construct
  source 1..13: mol_type = other DNA; note = RT Primer
atagagcccgcca

SEQ ID NO: 48 - DNA, 60 bp, synthetic construct
  source 1..60: mol_type = other DNA; note = RT Primer
tcgtcggcagcgtcagatgtgtataagagacagnnnnnnnnatagagcccgccannnnnn

SEQ ID NO: 49 - DNA, 13 bp, synthetic construct
  source 1..13: mol_type = other DNA; note = RT Primer
aagaaatggagga

SEQ ID NO: 50 - DNA, 60 bp, synthetic construct
  source 1..60: mol_type = other DNA; note = RT Primer
tcgtcggcagcgtcagatgtgtataagagacagnnnnnnnnaagaaatggaggannnnnn

SEQ ID NO: 51 - DNA, 13 bp, synthetic construct
  source 1..13: mol_type = other DNA; note = RT Primer
tcagcgctggtta

SEQ ID NO: 52 - DNA, 60 bp, synthetic construct
  source 1..60: mol_type = other DNA; note = RT Primer
tcgtcggcagcgtcagatgtgtataagagacagnnnnnnnntcagcgctggttannnnnn

SEQ ID NO: 53 - DNA, 13 bp, synthetic construct
  source 1..13: mol_type = other DNA; note = RT Primer
tgttgacgaataa

SEQ ID NO: 54 - DNA, 60 bp, synthetic construct
  source 1..60: mol_type = other DNA; note = RT Primer
tcgtcggcagcgtcagatgtgtataagagacagnnnnnnnntgttgacgaataannnnnn

SEQ ID NO: 55 - DNA, 13 bp, synthetic construct
  source 1..13: mol_type = other DNA; note = RT Primer
ttgcatcagcgca

SEQ ID NO: 56 - DNA, 60 bp, synthetic construct
  source 1..60: mol_type = other DNA; note = RT Primer
tcgtcggcagcgtcagatgtgtataagagacagnnnnnnnnttgcatcagcgcannnnnn

SEQ ID NO: 57 - DNA, 13 bp, synthetic construct
  source 1..13: mol_type = other DNA; note = RT Primer
tagccatatgaga

SEQ ID NO: 58 - DNA, 60 bp, synthetic construct
  source 1..60: mol_type = other DNA; note = RT Primer
tcgtcggcagcgtcagatgtgtataagagacagnnnnnnnntagccatatgagannnnnn

SEQ ID NO: 59 - DNA, 13 bp, synthetic construct
  source 1..13: mol_type = other DNA; note = RT Primer
tcctgcccctgta

SEQ ID NO: 60 - DNA, 60 bp, synthetic construct
  source 1..60: mol_type = other DNA; note = RT Primer
tcgtcggcagcgtcagatgtgtataagagacagnnnnnnnntcctgcccctgtannnnnn

SEQ ID NO: 61 - DNA, 13 bp, synthetic construct
  source 1..13: mol_type = other DNA; note = RT Primer
tggtataaaacca

SEQ ID NO: 62 - DNA, 60 bp, synthetic construct
  source 1..60: mol_type = other DNA; note = RT Primer
tcgtcggcagcgtcagatgtgtataagagacagnnnnnnnntggtataaaaccannnnnn

SEQ ID NO: 63 - DNA, 13 bp, synthetic construct
  source 1..13: mol_type = other DNA; note = RT Primer
ttaaagtcatgca

SEQ ID NO: 64 - DNA, 60 bp, synthetic construct
  source 1..60: mol_type = other DNA; note = RT Primer
tcgtcggcagcgtcagatgtgtataagagacagnnnnnnnnttaaagtcatgcannnnnn

SEQ ID NO: 65 - DNA, 13 bp, synthetic construct
  source 1..13: mol_type = other DNA; note = RT Primer
tagaactcagaaa

SEQ ID NO: 66 - DNA, 60 bp, synthetic construct
  source 1..60: mol_type = other DNA; note = RT Primer
tcgtcggcagcgtcagatgtgtataagagacagnnnnnnnntagaactcagaaannnnnn

SEQ ID NO: 67 - DNA, 13 bp, synthetic construct
  source 1..13: mol_type = other DNA; note = RT Primer
tcagcttaatcgc

SEQ ID NO: 68 - DNA, 60 bp, synthetic construct
  source 1..60: mol_type = other DNA; note = RT Primer
tcgtcggcagcgtcagatgtgtataagagacagnnnnnnnntcagcttaatcgcnnnnnn

SEQ ID NO: 69 - DNA, 13 bp, synthetic construct
  source 1..13: mol_type = other DNA; note = RT Primer
tggcttaagagac

SEQ ID NO: 70 - DNA, 60 bp, synthetic construct
  source 1..60: mol_type = other DNA; note = RT Primer
tcgtcggcagcgtcagatgtgtataagagacagnnnnnnnntggcttaagagacnnnnnn

SEQ ID NO: 71 - DNA, 13 bp, synthetic construct
  source 1..13: mol_type = other DNA; note = RT Primer
ttcacctgtcggc

SEQ ID NO: 72 - DNA, 60 bp, synthetic construct
  source 1..60: mol_type = other DNA; note = RT Primer
tcgtcggcagcgtcagatgtgtataagagacagnnnnnnnnttcacctgtcggcnnnnnn

SEQ ID NO: 73 - DNA, 13 bp, synthetic construct
  source 1..13: mol_type = other DNA; note = RT Primer
tatagtgatactc

SEQ ID NO: 74 - DNA, 60 bp, synthetic construct
  source 1..60: mol_type = other DNA; note = RT Primer
tcgtcggcagcgtcagatgtgtataagagacagnnnnnnnntatagtgatactcnnnnnn

SEQ ID NO: 75 - DNA, 13 bp, synthetic construct
  source 1..13: mol_type = other DNA; note = RT Primer
tcatcagcgaacc

SEQ ID NO: 76 - DNA, 60 bp, synthetic construct
  source 1..60: mol_type = other DNA; note = RT Primer
tcgtcggcagcgtcagatgtgtataagagacagnnnnnnnntcatcagcgaaccnnnnnn

SEQ ID NO: 77 - DNA, 13 bp, synthetic construct
  source 1..13: mol_type = other DNA; note = RT Primer
tgcgccatgggac

SEQ ID NO: 78 - DNA, 60 bp, synthetic construct
  source 1..60: mol_type = other DNA; note = RT Primer
tcgtcggcagcgtcagatgtgtataagagacagnnnnnnnntgcgccatgggacnnnnnn

SEQ ID NO: 79 - DNA, 13 bp, synthetic construct
  source 1..13: mol_type = other DNA; note = RT Primer
ttgttgaactccc

SEQ ID NO: 80 - DNA, 60 bp, synthetic construct
  source 1..60: mol_type = other DNA; note = RT Primer
tcgtcggcagcgtcagatgtgtataagagacagnnnnnnnnttgttgaactcccnnnnnn

SEQ ID NO: 81 - DNA, 13 bp, synthetic construct
  source 1..13: mol_type = other DNA; note = RT Primer
tatatacgcagtc

SEQ ID NO: 82 - DNA, 60 bp, synthetic construct
  source 1..60: mol_type = other DNA; note = RT Primer
tcgtcggcagcgtcagatgtgtataagagacagnnnnnnnntatatacgcagtcnnnnnn

SEQ ID NO: 83 - DNA, 13 bp, synthetic construct
  source 1..13: mol_type = other DNA; note = RT Primer
tcgcacgacgtac

SEQ ID NO: 84 - DNA, 60 bp, synthetic construct
  source 1..60: mol_type = other DNA; note = RT Primer
tcgtcggcagcgtcagatgtgtataagagacagnnnnnnnntcgcacgacgtacnnnnnn

SEQ ID NO: 85 - DNA, 13 bp, synthetic construct
  source 1..13: mol_type = other DNA; note = RT Primer
tgggacagaaggc

SEQ ID NO: 86 - DNA, 60 bp, synthetic construct
  source 1..60: mol_type = other DNA; note = RT Primer
tcgtcggcagcgtcagatgtgtataagagacagnnnnnnnntgggacagaaggcnnnnnn

SEQ ID NO: 87 - DNA, 13 bp, synthetic construct
  source 1..13: mol_type = other DNA; note = RT Primer
tttaacaagtatc

SEQ ID NO: 88 - DNA, 60 bp, synthetic construct
  source 1..60: mol_type = other DNA; note = RT Primer
tcgtcggcagcgtcagatgtgtataagagacagnnnnnnnntttaacaagtatcnnnnnn

SEQ ID NO: 89 - DNA, 13 bp, synthetic construct
  source 1..13: mol_type = other DNA; note = RT Primer
tactcctgggtac

SEQ ID NO: 90 - DNA, 60 bp, synthetic construct
  source 1..60: mol_type = other DNA; note = RT Primer
tcgtcggcagcgtcagatgtgtataagagacagnnnnnnnntactcctgggtacnnnnnn

SEQ ID NO: 91 - DNA, 13 bp, synthetic construct
  source 1..13: mol_type = other DNA; note = RT Primer
tcagaaaaccccc

SEQ ID NO: 92 - DNA, 60 bp, synthetic construct
  source 1..60: mol_type = other DNA; note = RT Primer
tcgtcggcagcgtcagatgtgtataagagacagnnnnnnnntcagaaaacccccnnnnnn

SEQ ID NO: 93 - DNA, 13 bp, synthetic construct
  source 1..13: mol_type = other DNA; note = RT Primer
tgcacgagcagtc

SEQ ID NO: 94 - DNA, 60 bp, synthetic construct
  source 1..60: mol_type = other DNA; note = RT Primer
tcgtcggcagcgtcagatgtgtataagagacagnnnnnnnntgcacgagcagtcnnnnnn

SEQ ID NO: 95 - DNA, 13 bp, synthetic construct
  source 1..13: mol_type = other DNA; note = RT Primer
ttattatgcttcc

SEQ ID NO: 96 - DNA, 60 bp, synthetic construct
  source 1..60: mol_type = other DNA; note = RT Primer
tcgtcggcagcgtcagatgtgtataagagacagnnnnnnnnttattatgcttccnnnnnn

SEQ ID NO: 97 - DNA, 13 bp, synthetic construct
  source 1..13: mol_type = other DNA; note = RT Primer
tagcgactgtatc

SEQ ID NO: 98 - DNA, 60 bp, synthetic construct
  source 1..60: mol_type = other DNA; note = RT Primer
tcgtcggcagcgtcagatgtgtataagagacagnnnnnnnntagcgactgtatcnnnnnn

SEQ ID NO: 99 - DNA, 13 bp, synthetic construct
  source 1..13: mol_type = other DNA; note = RT Primer
cccttgcgacagc

SEQ ID NO: 100 - DNA, 60 bp, synthetic construct
  source 1..60: mol_type = other DNA; note = RT Primer
tcgtcggcagcgtcagatgtgtataagagacagnnnnnnnncccttgcgacagcnnnnnn

SEQ ID NO: 101 - DNA, 13 bp, synthetic construct
  source 1..13: mol_type = other DNA; note = RT Primer
cgttagattgcac

SEQ ID NO: 102 - DNA, 60 bp, synthetic construct
  source 1..60: mol_type = other DNA; note = RT Primer
tcgtcggcagcgtcagatgtgtataagagacagnnnnnnnncgttagattgcacnnnnnn

SEQ ID NO: 103 - DNA, 13 bp, synthetic construct
  source 1..13: mol_type = other DNA; note = RT Primer
ctaaataccatcc

SEQ ID NO: 104 - DNA, 60 bp, synthetic construct
  source 1..60: mol_type = other DNA; note = RT Primer
tcgtcggcagcgtcagatgtgtataagagacagnnnnnnnnctaaataccatccnnnnnn

SEQ ID NO: 105 - DNA, 13 bp, synthetic construct
  source 1..13: mol_type = other DNA; note = RT Primer
catattttttagc

SEQ ID NO: 106 - DNA, 60 bp, synthetic construct
  source 1..60: mol_type = other DNA; note = RT Primer
tcgtcggcagcgtcagatgtgtataagagacagnnnnnnnncatattttttagcnnnnnn

SEQ ID NO: 107 - DNA, 13 bp, synthetic construct
  source 1..13: mol_type = other DNA; note = RT Primer
cccctaaacgctc

SEQ ID NO: 108 - DNA, 60 bp, synthetic construct
  source 1..60: mol_type = other DNA; note = RT Primer
tcgtcggcagcgtcagatgtgtataagagacagnnnnnnnncccctaaacgctcnnnnnn

SEQ ID NO: 109 - DNA, 13 bp, synthetic construct
  source 1..13: mol_type = other DNA; note = RT Primer
cgattgttaggac

SEQ ID NO: 110 - DNA, 60 bp, synthetic construct
  source 1..60: mol_type = other DNA; note = RT Primer
tcgtcggcagcgtcagatgtgtataagagacagnnnnnnnncgattgttaggacnnnnnn

SEQ ID NO: 111 - DNA, 13 bp, synthetic construct
  source 1..13: mol_type = other DNA; note = RT Primer
ctaccgagtctcc

SEQ ID NO: 112 - DNA, 60 bp, synthetic construct
  source 1..60: mol_type = other DNA; note = RT Primer
tcgtcggcagcgtcagatgtgtataagagacagnnnnnnnnctaccgagtctccnnnnnn

SEQ ID NO: 113 - DNA, 13 bp, synthetic construct
  source 1..13: mol_type = other DNA; note = RT Primer
cagaacaaattgc

SEQ ID NO: 114 - DNA, 60 bp, synthetic construct
  source 1..60: mol_type = other DNA; note = RT Primer
tcgtcggcagcgtcagatgtgtataagagacagnnnnnnnncagaacaaattgcnnnnnn

SEQ ID NO: 115 - DNA, 13 bp, synthetic construct
  source 1..13: mol_type = other DNA; note = RT Primer
ccttgatctcatc

SEQ ID NO: 116 - DNA, 60 bp, synthetic construct
  source 1..60: mol_type = other DNA; note = RT Primer
tcgtcggcagcgtcagatgtgtataagagacagnnnnnnnnccttgatctcatcnnnnnn

SEQ ID NO: 117 - DNA, 13 bp, synthetic construct
  source 1..13: mol_type = other DNA; note = RT Primer
cgccacgcgttac

SEQ ID NO: 118 - DNA, 60 bp, synthetic construct
  source 1..60: mol_type = other DNA; note = RT Primer
tcgtcggcagcgtcagatgtgtataagagacagnnnnnnnncgccacgcgttacnnnnnn

SEQ ID NO: 119 - DNA, 13 bp, synthetic construct
  source 1..13: mol_type = other DNA; note = RT Primer
ctatagatacggc

SEQ ID NO: 120 - DNA, 60 bp, synthetic construct
  source 1..60: mol_type = other DNA; note = RT Primer
tcgtcggcagcgtcagatgtgtataagagacagnnnnnnnnctatagatacggcnnnnnn

SEQ ID NO: 121 - DNA, 13 bp, synthetic construct
  source 1..13: mol_type = other DNA; note = RT Primer
catacccagccac

SEQ ID NO: 122 - DNA, 60 bp, synthetic construct
  source 1..60: mol_type = other DNA; note = RT Primer
tcgtcggcagcgtcagatgtgtataagagacagnnnnnnnncatacccagccacnnnnnn

SEQ ID NO: 123 - DNA, 13 bp, synthetic construct
  source 1..13: mol_type = other DNA; note = RT Primer
ccccggatgtacc

SEQ ID NO: 124 - DNA, 60 bp, synthetic construct
  source 1..60: mol_type = other DNA; note = RT Primer
tcgtcggcagcgtcagatgtgtataagagacagnnnnnnnnccccggatgtaccnnnnnn

SEQ ID NO: 125 - DNA, 13 bp, synthetic construct
  source 1..13: mol_type = other DNA; note = RT Primer
cgatctcggcttc

SEQ ID NO: 126 - DNA, 60 bp, synthetic construct
  source 1..60: mol_type = other DNA; note = RT Primer
tcgtcggcagcgtcagatgtgtataagagacagnnnnnnnncgatctcggcttcnnnnnn

SEQ ID NO: 127 - DNA, 13 bp, synthetic construct
  source 1..13: mol_type = other DNA; note = RT Primer
ctagccgctttgc

SEQ ID NO: 128 - DNA, 60 bp, synthetic construct
  source 1..60: mol_type = other DNA; note = RT Primer
tcgtcggcagcgtcagatgtgtataagagacagnnnnnnnnctagccgctttgcnnnnnn

SEQ ID NO: 129 - DNA, 13 bp, synthetic construct
  source 1..13: mol_type = other DNA; note = RT Primer
cctaaatacagcc

SEQ ID NO: 130 - DNA, 60 bp, synthetic construct
  source 1..60: mol_type = other DNA; note = RT Primer
tcgtcggcagcgtcagatgtgtataagagacagnnnnnnnncctaaatacagccnnnnnn

SEQ ID NO: 131 - DNA, 13 bp, synthetic construct
  source 1..13: mol_type = other DNA; note = RT Primer
cgggttttaccag

SEQ ID NO: 132 - DNA, 60 bp, synthetic construct
  source 1..60: mol_type = other DNA; note = RT Primer
tcgtcggcagcgtcagatgtgtataagagacagnnnnnnnncgggttttaccagnnnnnn

SEQ ID NO: 133 - DNA, 13 bp, synthetic construct
  source 1..13: mol_type = other DNA; note = RT Primer
catatccattgcg

SEQ ID NO: 134 - DNA, 60 bp, synthetic construct
  source 1..60: mol_type = other DNA; note = RT Primer
tcgtcggcagcgtcagatgtgtataagagacagnnnnnnnncatatccattgcgnnnnnn

SEQ ID NO: 135 - DNA, 13 bp, synthetic construct
  source 1..13: mol_type = other DNA; note = RT Primer
cctcccgagagtg

SEQ ID NO: 136 - DNA, 60 bp, synthetic construct
  source 1..60: mol_type = other DNA; note = RT Primer
tcgtcggcagcgtcagatgtgtataagagacagnnnnnnnncctcccgagagtgnnnnnn

SEQ ID NO: 137 - DNA, 13 bp, synthetic construct
  source 1..13: mol_type = other DNA; note = RT Primer
cgaatatctccgg

SEQ ID NO: 138 - DNA, 60 bp, synthetic construct
  source 1..60: mol_type = other DNA; note = RT Primer
tcgtcggcagcgtcagatgtgtataagagacagnnnnnnnncgaatatctccggnnnnnn

SEQ ID NO: 139 - DNA, 13 bp, synthetic construct
  source 1..13: mol_type = other DNA; note = RT Primer
ctactaacccacg

SEQ ID NO: 140 - DNA, 60 bp, synthetic construct
  source 1..60: mol_type = other DNA; note = RT Primer
tcgtcggcagcgtcagatgtgtataagagacagnnnnnnnnctactaacccacgnnnnnn

SEQ ID NO: 141 - DNA, 13 bp, synthetic construct
  source 1..13: mol_type = other DNA; note = RT Primer
caaatgatacaag

SEQ ID NO: 142 - DNA, 60 bp, synthetic construct
  source 1..60: mol_type = other DNA; note = RT Primer
tcgtcggcagcgtcagatgtgtataagagacagnnnnnnnncaaatgatacaagnnnnnn

SEQ ID NO: 143 - DNA, 13 bp, synthetic construct
  source 1..13: mol_type = other DNA; note = RT Primer
cccccattatttg

SEQ ID NO: 144 - DNA, 60 bp, synthetic construct
  source 1..60: mol_type = other DNA; note = RT Primer
tcgtcggcagcgtcagatgtgtataagagacagnnnnnnnncccccattatttgnnnnnn

SEQ ID NO: 145 - DNA, 13 bp, synthetic construct
  source 1..13: mol_type = other DNA; note = RT Primer
cgagtcgcgacgg

SEQ ID NO: 146 - DNA, 60 bp, synthetic construct
  source 1..60: mol_type = other DNA; note = RT Primer
tcgtcggcagcgtcagatgtgtataagagacagnnnnnnnncgagtcgcgacggnnnnnn

SEQ ID NO: 147 - DNA, 13 bp, synthetic construct
  source 1..13: mol_type = other DNA; note = RT Primer
gtggttgtggccg

SEQ ID NO: 148 - DNA, 60 bp, synthetic construct
  source 1..60: mol_type = other DNA; note = RT Primer
tcgtcggcagcgtcagatgtgtataagagacagnnnnnnnngtggttgtggccgnnnnnn

SEQ ID NO: 149 - DNA, 13 bp, synthetic construct
  source 1..13: mol_type = other DNA; note = RT Primer
gattcaaggaaag

SEQ ID NO: 150 - DNA, 60 bp, synthetic construct
  source 1..60: mol_type = other DNA; note = RT Primer
tcgtcggcagcgtcagatgtgtataagagacagnnnnnnnngattcaaggaaagnnnnnn

SEQ ID NO: 151 - DNA, 13 bp, synthetic construct
  source 1..13: mol_type = other DNA; note = RT Primer
gccaagatctggg

SEQ ID NO: 152 - DNA, 60 bp, synthetic construct
  source 1..60: mol_type = other DNA; note = RT Primer
tcgtcggcagcgtcagatgtgtataagagacagnnnnnnnngccaagatctgggnnnnnn

SEQ ID NO: 153 - DNA, 13 bp, synthetic construct
  source 1..13: mol_type = other DNA; note = RT Primer
gatactgtatcag

SEQ ID NO: 154 - DNA, 60 bp, synthetic construct
  source 1..60: mol_type = other DNA; note = RT Primer
tcgtcggcagcgtcagatgtgtataagagacagnnnnnnnngatactgtatcagnnnnnn

SEQ ID NO: 155 - DNA, 13 bp, synthetic construct
  source 1..13: mol_type = other DNA; note = RT Primer
gccgattcgtgcg

SEQ ID NO: 156 - DNA, 60 bp, synthetic construct
  source 1..60: mol_type = other DNA; note = RT Primer
tcgtcggcagcgtcagatgtgtataagagacagnnnnnnnngccgattcgtgcgnnnnnn

SEQ ID NO: 157 - DNA, 13 bp, synthetic construct
  source 1..13: mol_type = other DNA; note = RT Primer
gtgacaacgctgg

SEQ ID NO: 158 - DNA, 60 bp, synthetic construct
  source 1..60: mol_type = other DNA; note = RT Primer
tcgtcggcagcgtcagatgtgtataagagacagnnnnnnnngtgacaacgctggnnnnnn

SEQ ID NO: 159 - DNA, 13 bp, synthetic construct
  source 1..13: mol_type = other DNA; note = RT Primer
gggctgtcttatg

SEQ ID NO: 160 - DNA, 60 bp, synthetic construct
  source 1..60: mol_type = other DNA; note = RT Primer
tcgtcggcagcgtcagatgtgtataagagacagnnnnnnnngggctgtcttatgnnnnnn

SEQ ID NO: 161 - DNA, 13 bp, synthetic construct
  source 1..13: mol_type = other DNA; note = RT Primer
gaccctggcctgg

SEQ ID NO: 162 - DNA, 60 bp, synthetic construct
  source 1..60: mol_type = other DNA; note = RT Primer
tcgtcggcagcgtcagatgtgtataagagacagnnnnnnnngaccctggcctggnnnnnn

SEQ ID NO: 163 - DNA, 13 bp, synthetic construct
  source 1..13: mol_type = other DNA; note = RT Primer
gcgtacgcttatg

SEQ ID NO: 164 - DNA, 60 bp, synthetic construct
  source 1..60: mol_type = other DNA; note = RT Primer
tcgtcggcagcgtcagatgtgtataagagacagnnnnnnnngcgtacgcttatgnnnnnn

SEQ ID NO: 165 - DNA, 13 bp, synthetic construct
  source 1..13: mol_type = other DNA; note = RT Primer
gttgcacttaatg

SEQ ID NO: 166 - DNA, 60 bp, synthetic construct
  source 1..60: mol_type = other DNA; note = RT Primer
tcgtcggcagcgtcagatgtgtataagagacagnnnnnnnngttgcacttaatgnnnnnn

SEQ ID NO: 167 - DNA, 13 bp, synthetic construct
  source 1..13: mol_type = other DNA; note = RT Primer
ggacaaagagaag

SEQ ID NO: 168 - DNA, 60 bp, synthetic construct
  source 1..60: mol_type = other DNA; note = RT Primer
tcgtcggcagcgtcagatgtgtataagagacagnnnnnnnnggacaaagagaagnnnnnn

SEQ ID NO: 169 - DNA, 13 bp, synthetic construct
  source 1..13: mol_type = other DNA; note = RT Primer
gaaccacagcacg

SEQ ID NO: 170 - DNA, 60 bp, synthetic construct
  source 1..60: mol_type = other DNA; note = RT Primer
tcgtcggcagcgtcagatgtgtataagagacagnnnnnnnngaaccacagcacgnnnnnn

SEQ ID NO: 171 - DNA, 13 bp, synthetic construct
  source 1..13: mol_type = other DNA; note = RT Primer
gcttatctagagg

SEQ ID NO: 172 - DNA, 60 bp, synthetic construct
  source 1..60: mol_type = other DNA; note = RT Primer
tcgtcggcagcgtcagatgtgtataagagacagnnnnnnnngcttatctagaggnnnnnn

SEQ ID NO: 173 - DNA, 13 bp, synthetic construct
  source 1..13: mol_type = other DNA; note = RT Primer
gtaatgtggtcag

SEQ ID NO: 174 - DNA, 60 bp, synthetic construct
  source 1..60: mol_type = other DNA; note = RT Primer
tcgtcggcagcgtcagatgtgtataagagacagnnnnnnnngtaatgtggtcagnnnnnn

SEQ ID NO: 175 - DNA, 13 bp, synthetic construct
  source 1..13: mol_type = other DNA; note = RT Primer
ggcgcgcgagctg

SEQ ID NO: 176 - DNA, 60 bp, synthetic construct
  source 1..60: mol_type = other DNA; note = RT Primer
tcgtcggcagcgtcagatgtgtataagagacagnnnnnnnnggcgcgcgagctgnnnnnn

SEQ ID NO: 177 - DNA, 13 bp, synthetic construct
  source 1..13: mol_type = other DNA; note = RT Primer
gtccatgtgtgtg

SEQ ID NO: 178 - DNA, 60 bp, synthetic construct
  source 1..60: mol_type = other DNA; note = RT Primer
tcgtcggcagcgtcagatgtgtataagagacagnnnnnnnngtccatgtgtgtgnnnnnn

SEQ ID NO: 179 - DNA, 13 bp, synthetic construct
  source 1..13: mol_type = other DNA; note = RT Primer
gggaggaatccag

SEQ ID NO: 180 - DNA, 60 bp, synthetic construct
  source 1..60: mol_type = other DNA; note = RT Primer
tcgtcggcagcgtcagatgtgtataagagacagnnnnnnnngggaggaatccagnnnnnn

SEQ ID NO: 181 - DNA, 13 bp, synthetic construct
  source 1..13: mol_type = other DNA; note = RT Primer
gatcaaaataggg

SEQ ID NO: 182 - DNA, 60 bp, synthetic construct
  source 1..60: mol_type = other DNA; note = RT Primer
tcgtcggcagcgtcagatgtgtataagagacagnnnnnnnngatcaaaatagggnnnnnn

SEQ ID NO: 183 - DNA, 13 bp, synthetic construct
  source 1..13: mol_type = other DNA; note = RT Primer
gcaagactgccag

SEQ ID NO: 184 - DNA, 60 bp, synthetic construct
  source 1..60: mol_type = other DNA; note = RT Primer
tcgtcggcagcgtcagatgtgtataagagacagnnnnnnnngcaagactgccagnnnnnn

SEQ ID NO: 185 - DNA, 13 bp, synthetic construct
  source 1..13: mol_type = other DNA; note = RT Primer
gtaggatggaccg

SEQ ID NO: 186 - DNA, 60 bp, synthetic construct
  source 1..60: mol_type = other DNA; note = RT Primer
tcgtcggcagcgtcagatgtgtataagagacagnnnnnnnngtaggatggaccgnnnnnn

SEQ ID NO: 187 - DNA, 13 bp, synthetic construct
  source 1..13: mol_type = other DNA; note = RT Primer
ggatcggacctag

SEQ ID NO: 188 - DNA, 60 bp, synthetic construct
  source 1..60: mol_type = other DNA; note = RT Primer
tcgtcggcagcgtcagatgtgtataagagacagnnnnnnnnggatcggacctagnnnnnn

SEQ ID NO: 189 - DNA, 13 bp, synthetic construct
  source 1..13: mol_type = other DNA; note = RT Primer
gcccaaaggaatg

SEQ ID NO: 190 - DNA, 60 bp, synthetic construct
  source 1..60: mol_type = other DNA; note = RT Primer
tcgtcggcagcgtcagatgtgtataagagacagnnnnnnnngcccaaaggaatgnnnnnn

SEQ ID NO: 191 - DNA, 13 bp, synthetic construct
  source 1..13: mol_type = other DNA; note = RT Primer
gtacctaacgcag

SEQ ID NO: 192 - DNA, 60 bp, synthetic construct
  source 1..60: mol_type = other DNA; note = RT Primer
tcgtcggcagcgtcagatgtgtataagagacagnnnnnnnngtacctaacgcagnnnnnn

SEQ ID NO: 193 - DNA, 13 bp, synthetic construct
  source 1..13: mol_type = other DNA; note = RT Primer
ggtcaggttcagg

SEQ ID NO: 194 - DNA, 60 bp, synthetic construct
  source 1..60: mol_type = other DNA; note = RT Primer
tcgtcggcagcgtcagatgtgtataagagacagnnnnnnnnggtcaggttcaggnnnnnn

SEQ ID NO: 195 - DNA, 13 bp, synthetic construct
  source 1..13: mol_type = other DNA; note = RT Primer
aacctccaaccca

SEQ ID NO: 196 - DNA, 60 bp, synthetic construct
  source 1..60: mol_type = other DNA; note = RT Primer
tcgtcggcagcgtcagatgtgtataagagacagnnnnnnnnaacctccaacccannnnnn

SEQ ID NO: 197 - DNA, 13 bp, synthetic construct
  source 1..13: mol_type = other DNA; note = RT Primer
acgtttcagccaa

SEQ ID NO: 198 - DNA, 60 bp, synthetic construct
  source 1..60: mol_type = other DNA; note = RT Primer
tcgtcggcagcgtcagatgtgtataagagacagnnnnnnnnacgtttcagccaannnnnn

SEQ ID NO: 199 - DNA, 13 bp, synthetic construct
  source 1..13: mol_type = other DNA; note = RT Primer
ataaggttagata

SEQ ID NO: 200 - DNA, 60 bp, synthetic construct
  source 1..60: mol_type = other DNA; note = RT Primer
tcgtcggcagcgtcagatgtgtataagagacagnnnnnnnnataaggttagatannnnnn

SEQ ID NO: 201 - DNA, 13 bp, synthetic construct
  source 1..13: mol_type = other DNA; note = RT Primer
aatataccagaca

SEQ ID NO: 202 - DNA, 60 bp, synthetic construct
  source 1..60: mol_type = other DNA; note = RT Primer
tcgtcggcagcgtcagatgtgtataagagacagnnnnnnnnaatataccagacannnnnn

SEQ ID NO: 203 - DNA, 13 bp, synthetic construct
  source 1..13: mol_type = other DNA; note = RT Primer
aggagagactgta

SEQ ID NO: 204 - DNA, 60 bp, synthetic construct
  source 1..60: mol_type = other DNA; note = RT Primer
tcgtcggcagcgtcagatgtgtataagagacagnnnnnnnnaggagagactgtannnnnn

SEQ ID NO: 205 - DNA, 13 bp, synthetic construct
  source 1..13: mol_type = other DNA; note = RT Primer
actcatcttagca

SEQ ID NO: 206 - DNA, 60 bp, synthetic construct
  source 1..60: mol_type = other DNA; note = RT Primer
tcgtcggcagcgtcagatgtgtataagagacagnnnnnnnnactcatcttagcannnnnn

SEQ ID NO: 207 - DNA, 13 bp, synthetic construct
  source 1..13: mol_type = other DNA; note = RT Primer
aaatttggccgta

SEQ ID NO: 208 - DNA, 60 bp, synthetic construct
  source 1..60: mol_type = other DNA; note = RT Primer
tcgtcggcagcgtcagatgtgtataagagacagnnnnnnnnaaatttggccgtannnnnn

SEQ ID NO: 209 - DNA, 13 bp, synthetic construct
  source 1..13: mol_type = other DNA; note = RT Primer
attaaggaagtaa

SEQ ID NO: 210 - DNA, 60 bp, synthetic construct
  source 1..60: mol_type = other DNA; note = RT Primer
tcgtcggcagcgtcagatgtgtataagagacagnnnnnnnnattaaggaagtaannnnnn

SEQ ID NO: 211 - DNA, 13 bp, synthetic construct
  source 1..13: mol_type = other DNA; note = RT Primer
aaccgatccagaa

SEQ ID NO: 212 - DNA, 60 bp, synthetic construct
  source 1..60: mol_type = other DNA; note = RT Primer
tcgtcggcagcgtcagatgtgtataagagacagnnnnnnnnaaccgatccagaannnnnn

SEQ ID NO: 213 - DNA, 13 bp, synthetic construct
  source 1..13: mol_type = other DNA; note = RT Primer
acaacgggactga

SEQ ID NO: 214 - DNA, 60 bp, synthetic construct
  source 1..60: mol_type = other DNA; note = RT Primer
tcgtcggcagcgtcagatgtgtataagagacagnnnnnnnnacaacgggactgannnnnn

SEQ ID NO: 215 - DNA, 13 bp, synthetic construct
  source 1..13: mol_type = other DNA; note = RT Primer
atccagtacaaca

SEQ ID NO: 216 - DNA, 60 bp, synthetic construct
  source 1..60: mol_type = other DNA; note = RT Primer
tcgtcggcagcgtcagatgtgtataagagacagnnnnnnnnatccagtacaacannnnnn

SEQ ID NO: 217 - DNA, 13 bp, synthetic construct
  source 1..13: mol_type = other DNA; note = RT Primer
agaggacgatgca

SEQ ID NO: 218 - DNA, 60 bp, synthetic construct
  source 1..60: mol_type = other DNA; note = RT Primer
tcgtcggcagcgtcagatgtgtataagagacagnnnnnnnnagaggacgatgcannnnnn

SEQ ID NO: 219 - DNA, 13 bp, synthetic construct
  source 1..13: mol_type = other DNA; note = RT Primer
aaagggtcgctta

SEQ ID NO: 220 - DNA, 60 bp, synthetic construct
  source 1..60: mol_type = other DNA; note = RT Primer
tcgtcggcagcgtcagatgtgtataagagacagnnnnnnnnaaagggtcgcttannnnnn

SEQ ID NO: 221 - DNA, 13 bp, synthetic construct
  source 1..13: mol_type = other DNA; note = RT Primer
acccatgtagcaa

SEQ ID NO: 222 - DNA, 60 bp, synthetic construct
  source 1..60: mol_type = other DNA; note = RT Primer
tcgtcggcagcgtcagatgtgtataagagacagnnnnnnnnacccatgtagcaannnnnn

SEQ ID NO: 223 - DNA, 13 bp, synthetic construct
  source 1..13: mol_type = other DNA; note = RT Primer
agtgaagtggaca

SEQ ID NO: 224 - DNA, 60 bp, synthetic construct
  source 1..60: mol_type = other DNA; note = RT Primer
tcgtcggcagcgtcagatgtgtataagagacagnnnnnnnnagtgaagtggacannnnnn

SEQ ID NO: 225 - DNA, 13 bp, synthetic construct
  source 1..13: mol_type = other DNA; note = RT Primer
ataataattacaa

SEQ ID NO: 226 - DNA, 60 bp, synthetic construct
  source 1..60: mol_type = other DNA; note = RT Primer
tcgtcggcagcgtcagatgtgtataagagacagnnnnnnnnataataattacaannnnnn

SEQ ID NO: 227 - DNA, 13 bp, synthetic construct
  source 1..13: mol_type = other DNA; note = RT Primer
agctgtaacgtga

SEQ ID NO: 228 - DNA, 60 bp, synthetic construct
  source 1..60: mol_type = other DNA; note = RT Primer
tcgtcggcagcgtcagatgtgtataagagacagnnnnnnnnagctgtaacgtgannnnnn

SEQ ID NO: 229 - DNA, 13 bp, synthetic construct
  source 1..13: mol_type = other DNA; note = RT Primer
aaaaagctgcgta

SEQ ID NO: 230 - DNA, 60 bp, synthetic construct
  source 1..60: mol_type = other DNA; note = RT Primer
tcgtcggcagcgtcagatgtgtataagagacagnnnnnnnnaaaaagctgcgtannnnnn

SEQ ID NO: 231 - DNA, 13 bp, synthetic construct
  source 1..13: mol_type = other DNA; note = RT Primer
acgcgcaaacaga

SEQ ID NO: 232 - DNA, 60 bp, synthetic construct
  source 1..60: mol_type = other DNA; note = RT Primer
tcgtcggcagcgtcagatgtgtataagagacagnnnnnnnnacgcgcaaacagannnnnn

SEQ ID NO: 233 - DNA, 13 bp, synthetic construct
  source 1..13: mol_type = other DNA; note = RT Primer
ataattttcccca

SEQ ID NO: 234 - DNA, 60 bp, synthetic construct
  source 1..60: mol_type = other DNA; note = RT Primer
tcgtcggcagcgtcagatgtgtataagagacagnnnnnnnnataattttccccannnnnn

SEQ ID NO: 235 - DNA, 13 bp, synthetic construct
  source 1..13: mol_type = other DNA; note = RT Primer
aggatactggtta

SEQ ID NO: 236 - DNA, 60 bp, synthetic construct
  source 1..60: mol_type = other DNA; note = RT Primer
tcgtcggcagcgtcagatgtgtataagagacagnnnnnnnnaggatactggttannnnnn

SEQ ID NO: 237 - DNA, 13 bp, synthetic construct
  source 1..13: mol_type = other DNA; note = RT Primer
aagggggaagcga

SEQ ID NO: 238 - DNA, 60 bp, synthetic construct
  source 1..60: mol_type = other DNA; note = RT Primer
tcgtcggcagcgtcagatgtgtataagagacagnnnnnnnnaagggggaagcgannnnnn

SEQ ID NO: 239 - DNA, 13 bp, synthetic construct
  source 1..13: mol_type = other DNA; note = RT Primer
acaacgccaaaaa

SEQ ID NO: 240 - DNA, 60 bp, synthetic construct
  source 1..60: mol_type = other DNA; note = RT Primer
tcgtcggcagcgtcagatgtgtataagagacagnnnnnnnnacaacgccaaaaannnnnn

SEQ ID NO: 241 - DNA, 13 bp, synthetic construct
  source 1..13: mol_type = other DNA; note = RT Primer
aacgaagcctgta

SEQ ID NO: 242 - DNA, 60 bp, synthetic construct
  source 1..60: mol_type = other DNA; note = RT Primer
tcgtcggcagcgtcagatgtgtataagagacagnnnnnnnnaacgaagcctgtannnnnn

SEQ ID NO: 243 - DNA, 13 bp, synthetic construct
  source 1..13: mol_type = other DNA; note = RT Primer
ttacgttgccaga

SEQ ID NO: 244 - DNA, 60 bp, synthetic construct
  source 1..60: mol_type = other DNA; note = RT Primer
tcgtcggcagcgtcagatgtgtataagagacagnnnnnnnnttacgttgccagannnnnn

SEQ ID NO: 245 - DNA, 13 bp, synthetic construct
  source 1..13: mol_type = other DNA; note = RT Primer
taattaacttata

SEQ ID NO: 246 - DNA, 60 bp, synthetic construct
  source 1..60: mol_type = other DNA; note = RT Primer
tcgtcggcagcgtcagatgtgtataagagacagnnnnnnnntaattaacttatannnnnn

SEQ ID NO: 247 - DNA, 13 bp, synthetic construct
  source 1..13: mol_type = other DNA; note = RT Primer
tctcggtatggga

SEQ ID NO: 248 - DNA, 60 bp, synthetic construct
  source 1..60: mol_type = other DNA; note = RT Primer
tcgtcggcagcgtcagatgtgtataagagacagnnnnnnnntctcggtatgggannnnnn

SEQ ID NO: 249 - DNA, 13 bp, synthetic construct
  source 1..13: mol_type = other DNA; note = RT Primer
ttcgtgtgaatta

SEQ ID NO: 250 - DNA, 60 bp, synthetic construct
  source 1..60: mol_type = other DNA; note = RT Primer
tcgtcggcagcgtcagatgtgtataagagacagnnnnnnnnttcgtgtgaattannnnnn

SEQ ID NO: 251 - DNA, 13 bp, synthetic construct
  source 1..13: mol_type = other DNA; note = RT Primer
tataattagttca

SEQ ID NO: 252 - DNA, 60 bp, synthetic construct
  source 1..60: mol_type = other DNA; note = RT Primer
tcgtcggcagcgtcagatgtgtataagagacagnnnnnnnntataattagttcannnnnn

SEQ ID NO: 253 - DNA, 13 bp, synthetic construct
  source 1..13: mol_type = other DNA; note = RT Primer
tggctaattccta

SEQ ID NO: 254 - DNA, 60 bp, synthetic construct
  source 1..60: mol_type = other DNA; note = RT Primer
tcgtcggcagcgtcagatgtgtataagagacagnnnnnnnntagaaggtcgtgannnnnn

SEQ ID NO: 255 - DNA, 13 bp, synthetic construct
  source 1..13: mol_type = other DNA; note = RT Primer
tagaaggtcgtga

SEQ ID NO: 256 - DNA, 60 bp, synthetic construct; identical to SEQ ID NO: 254
  source 1..60: mol_type = other DNA; note = RT Primer

SEQ ID NO: 257 - DNA, 13 bp, synthetic construct
  source 1..13: mol_type = other DNA; note = RT Primer
tcgtgagggggta

SEQ ID NO: 258 - DNA, 60 bp, synthetic construct
  source 1..60: mol_type = other DNA; note = RT Primer
tcgtcggcagcgtcagatgtgtataagagacagnnnnnnnntcgtgagggggtannnnnn

SEQ ID NO: 259 - DNA, 13 bp, synthetic construct
  source 1..13: mol_type = other DNA; note = RT Primer
ttaggctaaaggc

SEQ ID NO: 260 - DNA, 60 bp, synthetic construct
  source 1..60: mol_type = other DNA; note = RT Primer
tcgtcggcagcgtcagatgtgtataagagacagnnnnnnnnttaggctaaaggcnnnnnn

SEQ ID NO: 261 - DNA, 13 bp, synthetic construct
  source 1..13: mol_type = other DNA; note = RT Primer
tgaagtggcttcc

SEQ ID NO: 262 - DNA, 60 bp, synthetic construct
  source 1..60: mol_type = other DNA; note = RT Primer
tcgtcggcagcgtcagatgtgtataagagacagnnnnnnnntgaagtggcttccnnnnnn

SEQ ID NO: 263 - DNA, 13 bp, synthetic construct
  source 1..13: mol_type = other DNA; note = RT Primer
tatcgcatagcgc

SEQ ID NO: 264 - DNA, 60 bp, synthetic construct
  source 1..60: mol_type = other DNA; note = RT Primer
tcgtcggcagcgtcagatgtgtataagagacagnnnnnnnntatcgcatagcgcnnnnnn

SEQ ID NO: 265 - DNA, 13 bp, synthetic construct
  source 1..13: mol_type = other DNA; note = RT Primer
tctcctaagctcc

SEQ ID NO: 266 - DNA, 60 bp, synthetic construct
  source 1..60: mol_type = other DNA; note = RT Primer
tcgtcggcagcgtcagatgtgtataagagacagnnnnnnnntctcctaagctccnnnnnn

SEQ ID NO: 267 - DNA, 13 bp, synthetic construct
  source 1..13: mol_type = other DNA; note = RT Primer
taaatcatgattc

SEQ ID NO: 268 - DNA, 60 bp, synthetic construct
  source 1..60: mol_type = other DNA; note = RT Primer
tcgtcggcagcgtcagatgtgtataagagacagnnnnnnnntaaatcatgattcnnnnnn

SEQ ID NO: 269 - DNA, 13 bp, synthetic construct
  source 1..13: mol_type = other DNA; note = RT Primer
tcgcatacggaac

SEQ ID NO: 270 - DNA, 60 bp, synthetic construct
  source 1..60: mol_type = other DNA; note = RT Primer
tcgtcggcagcgtcagatgtgtataagagacagnnnnnnnntcgcatacggaacnnnnnn

SEQ ID NO: 271 - DNA, 13 bp, synthetic construct
  source 1..13: mol_type = other DNA; note = RT Primer
ttgcagccacagc

SEQ ID NO: 272 - DNA, 60 bp, synthetic construct
  source 1..60: mol_type = other DNA; note = RT Primer
tcgtcggcagcgtcagatgtgtataagagacagnnnnnnnnttgcagccacagcnnnnnn

SEQ ID NO: 273 - DNA, 13 bp, synthetic construct
  source 1..13: mol_type = other DNA; note = RT Primer
tacctggtgcttc

SEQ ID NO: 274 - DNA, 60 bp, synthetic construct
  source 1..60: mol_type = other DNA; note = RT Primer
tcgtcggcagcgtcagatgtgtataagagacagnnnnnnnntacctggtgcttcnnnnnn

SEQ ID NO: 275 - DNA, 13 bp, synthetic construct
  source 1..13: mol_type = other DNA; note = RT Primer
tgttttcataagc

SEQ ID NO: 276 - DNA, 60 bp, synthetic construct
  source 1..60: mol_type = other DNA; note = RT Primer
tcgtcggcagcgtcagatgtgtataagagacagnnnnnnnntgttttcataagcnnnnnn

SEQ ID NO: 277 - DNA, 13 bp, synthetic construct
  source 1..13: mol_type = other DNA; note = RT Primer
taaactgagatgc

SEQ ID NO: 278 - DNA, 60 bp, synthetic construct
  source 1..60: mol_type = other DNA; note = RT Primer
tcgtcggcagcgtcagatgtgtataagagacagnnnnnnnntaaactgagatgcnnnnnn

SEQ ID NO: 279 - DNA, 13 bp, synthetic construct
  source 1..13: mol_type = other DNA; note = RT Primer
tcagtgcgctgtc

SEQ ID NO: 280 - DNA, 60 bp, synthetic construct
  source 1..60: mol_type = other DNA; note = RT Primer
tcgtcggcagcgtcagatgtgtataagagacagnnnnnnnntcagtgcgctgtcnnnnnn

SEQ ID NO: 281 - DNA, 13 bp, synthetic construct
  source 1..13: mol_type = other DNA; note = RT Primer
ttgagtgggccac

SEQ ID NO: 282 - DNA, 60 bp, synthetic construct
  source 1..60: mol_type = other DNA; note = RT Primer
tcgtcggcagcgtcagatgtgtataagagacagnnnnnnnnttgagtgggccacnnnnnn

SEQ ID NO: 283 - DNA, 13 bp, synthetic construct
  source 1..13: mol_type = other DNA; note = RT Primer
tattctctcaacc

SEQ ID NO: 284 - DNA, 60 bp, synthetic construct
  source 1..60: mol_type = other DNA; note = RT Primer
tcgtcggcagcgtcagatgtgtataagagacagnnnnnnnntattctctcaaccnnnnnn

SEQ ID NO: 285 - DNA, 13 bp, synthetic construct
  source 1..13: mol_type = other DNA; note = RT Primer
tcctcctttacgc

SEQ ID NO: 286 - DNA, 60 bp, synthetic construct
  source 1..60: mol_type = other DNA; note = RT Primer
tcgtcggcagcgtcagatgtgtataagagacagnnnnnnnntcctcctttacgcnnnnnn

SEQ ID NO: 287 - DNA, 13 bp, synthetic construct
  source 1..13: mol_type = other DNA; note = RT Primer
tagagaactttac

SEQ ID NO: 288 - DNA, 60 bp, synthetic construct
  source 1..60: mol_type = other DNA; note = RT Primer
tcgtcggcagcgtcagatgtgtataagagacagnnnnnnnntagagaactttacnnnnnn

SEQ ID NO: 289 - DNA, 13 bp, synthetic construct
  source 1..13: mol_type = other DNA; note = RT Primer
ttactaccaggtc

SEQ ID NO: 290 - DNA, 60 bp, synthetic construct
  source 1..60: mol_type = other DNA; note = RT Primer
tcgtcggcagcgtcagatgtgtataagagacagnnnnnnnnttactaccaggtcnnnnnn

SEQ ID NO: 291 - DNA, 13 bp, synthetic construct
  source 1..13: mol_type = other DNA; note = RT Primer
cattgctacatcc

SEQ ID NO: 292 - DNA, 60 bp, synthetic construct
  source 1..60: mol_type = other DNA; note = RT Primer
tcgtcggcagcgtcagatgtgtataagagacagnnnnnnnncattgctacatccnnnnnn

SEQ ID NO: 293 - DNA, 13 bp, synthetic construct
  source 1..13: mol_type = other DNA; note = RT Primer
cggagtcacacgc

SEQ ID NO: 294 - DNA, 60 bp, synthetic construct
  source 1..60: mol_type = other DNA; note = RT Primer
tcgtcggcagcgtcagatgtgtataagagacagnnnnnnnncggagtcacacgcnnnnnn

SEQ ID NO: 295 - DNA, 13 bp, synthetic construct
  source 1..13: mol_type = other DNA; note = RT Primer
ccggtggagctcc

SEQ ID NO: 296 - DNA, 60 bp, synthetic construct
  source 1..60: mol_type = other DNA; note = RT Primer
tcgtcggcagcgtcagatgtgtataagagacagnnnnnnnnccggtggagctccnnnnnn

SEQ ID NO: 297 - DNA, 13 bp, synthetic construct
  source 1..13: mol_type = other DNA; note = RT Primer
cttcggcccaatc

SEQ ID NO: 298 - DNA, 60 bp, synthetic construct
  source 1..60: mol_type = other DNA; note = RT Primer
tcgtcggcagcgtcagatgtgtataagagacagnnnnnnnncttcggcccaatcnnnnnn

SEQ ID NO: 299 - DNA, 13 bp, synthetic construct
  source 1..13: mol_type = other DNA; note = RT Primer
cacgagacacccc

SEQ ID NO: 300 - DNA, 60 bp, synthetic construct
  source 1..60: mol_type = other DNA; note = RT Primer
tcgtcggcagcgtcagatgtgtataagagacagnnnnnnnncacgagacaccccnnnnnn

SEQ ID NO: 301 - DNA, 13 bp, synthetic construct
  source 1..13: mol_type = other DNA; note = RT Primer
cgtctagacaaac

SEQ ID NO: 302 - DNA, 60 bp, synthetic construct
  source 1..60: mol_type = other DNA; note = RT Primer
tcgtcggcagcgtcagatgtgtataagagacagnnnnnnnncgtctagacaaacnnnnnn

SEQ ID NO: 303 - DNA, 13 bp, synthetic construct
  source 1..13: mol_type = other DNA; note = RT Primer
caccgtacgttgc

SEQ ID NO: 304 - DNA, 60 bp, synthetic construct
  source 1..60: mol_type = other DNA; note = RT Primer
tcgtcggcagcgtcagatgtgtataagagacagnnnnnnnncaccgtacgttgcnnnnnn

SEQ ID NO: 305 - DNA, 13 bp, synthetic construct
  source 1..13: mol_type = other DNA; note = RT Primer
cctaaccctctcc

SEQ ID NO: 306 - DNA, 60 bp, synthetic construct
  source 1..60: mol_type = other DNA; note = RT Primer
tcgtcggcagcgtcagatgtgtataagagacagnnnnnnnncctaaccctctccnnnnnn

SEQ ID NO: 307 - DNA, 13 bp, synthetic construct
  source 1..13: mol_type = other DNA; note = RT Primer
ctagatttaactc

SEQ ID NO: 308 - DNA, 60 bp, synthetic construct
  source 1..60: mol_type = other DNA; note = RT Primer
tcgtcggcagcgtcagatgtgtataagagacagnnnnnnnnctagatttaactcnnnnnn

SEQ ID NO: 309 - DNA, 13 bp, synthetic construct
  source 1..13: mol_type = other DNA; note = RT Primer
cagattagagtac

SEQ ID NO: 310 - DNA, 60 bp, synthetic construct
  source 1..60: mol_type = other DNA; note = RT Primer
tcgtcggcagcgtcagatgtgtataagagacagnnnnnnnncagattagagtacnnnnnn

SEQ ID NO: 311 - DNA, 13 bp, synthetic construct
  source 1..13: mol_type = other DNA; note = RT Primer
cgggacgttttcc

SEQ ID NO: 312 - DNA, 60 bp, synthetic construct
  source 1..60: mol_type = other DNA; note = RT Primer
tcgtcggcagcgtcagatgtgtataagagacagnnnnnnnncgggacgttttccnnnnnn

SEQ ID NO: 313 - DNA, 13 bp, synthetic construct
  source 1..13: mol_type = other DNA; note = RT Primer
ctataccaagaac

SEQ ID NO: 314 - DNA, 60 bp, synthetic construct
  source 1..60: mol_type = other DNA; note = RT Primer
tcgtcggcagcgtcagatgtgtataagagacagnnnnnnnnctataccaagaacnnnnnn

SEQ ID NO: 315 - DNA, 13 bp, synthetic construct
  source 1..13: mol_type = other DNA; note = RT Primer
ccggcgagccagc

SEQ ID NO: 316 - DNA, 60 bp, synthetic construct
  source 1..60: mol_type = other DNA; note = RT Primer
tcgtcggcagcgtcagatgtgtataagagacagnnnnnnnnccggcgagccagcnnnnnn

SEQ ID NO: 317 - DNA, 13 bp, synthetic construct
  source 1..13: mol_type = other DNA; note = RT Primer
catttgaaaacgc

SEQ ID NO: 318 - DNA, 60 bp, synthetic construct
  source 1..60: mol_type = other DNA; note = RT Primer
tcgtcggcagcgtcagatgtgtataagagacagnnnnnnnncatttgaaaacgcnnnnnn

SEQ ID NO: 319 - DNA, 13 bp, synthetic construct
  source 1..13: mol_type = other DNA; note = RT Primer
cccctttctggac

SEQ ID NO: 320 - DNA, 60 bp, synthetic construct
  source 1..60: mol_type = other DNA; note = RT Primer
tcgtcggcagcgtcagatgtgtataagagacagnnnnnnnncccctttctggacnnnnnn

SEQ ID NO: 321 - DNA, 13 bp, synthetic construct
  source 1..13: mol_type = other DNA; note = RT Primer
ctgactgcagcgc

SEQ ID NO: 322 - DNA, 60 bp, synthetic construct
  source 1..60: mol_type = other DNA; note = RT Primer
tcgtcggcagcgtcagatgtgtataagagacagnnnnnnnnctgactgcagcgcnnnnnn

SEQ ID NO: 323 - DNA, 13 bp, synthetic construct
  source 1..13: mol_type = other DNA; note = RT Primer
cacgatcaagttg

SEQ ID NO: 324 - DNA, 60 bp, synthetic construct
  source 1..60: mol_type = other DNA; note = RT Primer
tcgtcggcagcgtcagatgtgtataagagacagnnnnnnnncacgatcaagttgnnnnnn

SEQ ID NO: 325 - DNA, 13 bp, synthetic construct
  source 1..13: mol_type = other DNA; note = RT Primer
cgaaatgctgacg

SEQ ID NO: 326 - DNA, 60 bp, synthetic construct
  source 1..60: mol_type = other DNA; note = RT Primer
tcgtcggcagcgtcagatgtgtataagagacagnnnnnnnncgaaatgctgacgnnnnnn

SEQ ID NO: 327 - DNA, 13 bp, synthetic construct
  source 1..13: mol_type = other DNA; note = RT Primer
cgctacctgaagg

SEQ ID NO: 328 - DNA, 60 bp, synthetic construct
  source 1..60: mol_type = other DNA; note = RT Primer
tcgtcggcagcgtcagatgtgtataagagacagnnnnnnnncgctacctgaaggnnnnnn

SEQ ID NO: 329 - DNA, 13 bp, synthetic construct
  source 1..13: mol_type = other DNA; note = RT Primer
caggaactacacg

SEQ ID NO: 330 - DNA, 60 bp, synthetic construct
  source 1..60: mol_type = other DNA; note = RT Primer
tcgtcggcagcgtcagatgtgtataagagacagnnnnnnnncaggaactacacgnnnnnn

SEQ ID NO: 331 - DNA, 13 bp, synthetic construct
  source 1..13: mol_type = other DNA; note = RT Primer
ctcggccggtcag

SEQ ID NO: 332 - DNA, 60 bp, synthetic construct
  source 1..60: mol_type = other DNA; note = RT Primer
tcgtcggcagcgtcagatgtgtataagagacagnnnnnnnnctcggccggtcagnnnnnn

SEQ ID NO: 333 - DNA, 13 bp, synthetic construct
  source 1..13: mol_type = other DNA; note = RT Primer
cttttccagcgtg

SEQ ID NO: 334 - DNA, 60 bp, synthetic construct
  source 1..60: mol_type = other DNA; note = RT Primer
tcgtcggcagcgtcagatgtgtataagagacagnnnnnnnncttttccagcgtgnnnnnn

SEQ ID NO: 335 - DNA, 13 bp, synthetic construct
  source 1..13: mol_type = other DNA; note = RT Primer
caatcgtagccgg

SEQ ID NO: 336 - DNA, 60 bp, synthetic construct
  source 1..60: mol_type = other DNA; note = RT Primer
tcgtcggcagcgtcagatgtgtataagagacagnnnnnnnncaatcgtagccggnnnnnn

SEQ ID NO: 337 - DNA, 13 bp, synthetic construct
  source 1..13: mol_type = other DNA; note = RT Primer
cgtaaactacgag

SEQ ID NO: 338 - DNA, 60 bp, synthetic construct
  source 1..60: mol_type = other DNA; note = RT Primer
tcgtcggcagcgtcagatgtgtataagagacagnnnnnnnncgtaaactacgagnnnnnn

SEQ ID NO: 339 - DNA, 13 bp, synthetic construct
  source 1..13: mol_type = other DNA; note = RT Primer
gacgagtatctgg

SEQ ID NO: 340 - DNA, 60 bp, synthetic construct
  source 1..60: mol_type = other DNA; note = RT Primer
tcgtcggcagcgtcagatgtgtataagagacagnnnnnnnngacgagtatctggnnnnnn

SEQ ID NO: 341 - DNA, 13 bp, synthetic construct
  source 1..13: mol_type = other DNA; note = RT Primer
gcaaagggtggtg

SEQ ID NO: 342 - DNA, 60 bp, synthetic construct
  source 1..60: mol_type = other DNA; note = RT Primer
tcgtcggcagcgtcagatgtgtataagagacagnnnnnnnngcaaagggtggtgnnnnnn

SEQ ID NO: 343 - DNA, 13 bp, synthetic construct
  source 1..13: mol_type = other DNA; note = RT Primer
gtaggtgagaaag

SEQ ID NO: 344 - DNA, 60 bp, synthetic construct
  source 1..60: mol_type = other DNA; note = RT Primer
tcgtcggcagcgtcagatgtgtataagagacagnnnnnnnngtaggtgagaaagnnnnnn

SEQ ID NO: 345 - DNA, 13 bp, synthetic construct
  source 1..13: mol_type = other DNA; note = RT Primer
gagcggattagtg

SEQ ID NO: 346 - DNA, 60 bp, synthetic construct
  source 1..60: mol_type = other DNA; note = RT Primer
tcgtcggcagcgtcagatgtgtataagagacagnnnnnnnngagcggattagtgnnnnnn

SEQ ID NO: 347 - DNA, 13 bp, synthetic construct
  source 1..13: mol_type = other DNA; note = RT Primer
ggtttgtatgtcg

SEQ ID NO: 348 - DNA, 60 bp, synthetic construct
  source 1..60: mol_type = other DNA; note = RT Primer
tcgtcggcagcgtcagatgtgtataagagacagnnnnnnnnggtttgtatgtcgnnnnnn

SEQ ID NO: 349 - DNA, 13 bp, synthetic construct
  source 1..13: mol_type = other DNA; note = RT Primer
gaacacatctgag

SEQ ID NO: 350 - DNA, 60 bp, synthetic construct
  source 1..60: mol_type = other DNA; note = RT Primer
tcgtcggcagcgtcagatgtgtataagagacagnnnnnnnngaacacatctgagnnnnnn

SEQ ID NO: 351 - DNA, 13 bp, synthetic construct
  source 1..13: mol_type = other DNA; note = RT Primer
gctaccaatgacg

SEQ ID NO: 352 - DNA, 60 bp, synthetic construct
  source 1..60: mol_type = other DNA; note = RT Primer
tcgtcggcagcgtcagatgtgtataagagacagnnnnnnnngctaccaatgacgnnnnnn

SEQ ID NO: 353 - DNA, 13 bp, synthetic construct
  source 1..13: mol_type = other DNA; note = RT Primer
gtgagggctcggg

SEQ ID NO: 354 - DNA, 60 bp, synthetic construct
  source 1..60: mol_type = other DNA; note = RT Primer
tcgtcggcagcgtcagatgtgtataagagacagnnnnnnnngtgagggctcgggnnnnnn

SEQ ID NO: 355 - DNA, 13 bp, synthetic construct
  source 1..13: mol_type = other DNA; note = RT Primer
gaaatgtgtcgcg

SEQ ID NO: 356 - DNA, 60 bp, synthetic construct
  source 1..60: mol_type = other DNA; note = RT Primer
tcgtcggcagcgtcagatgtgtataagagacagnnnnnnnngaaatgtgtcgcgnnnnnn

SEQ ID NO: 357 - DNA, 13 bp, synthetic construct
  source 1..13: mol_type = other DNA; note = RT Primer
ggcccacaccggg

SEQ ID NO: 358 - DNA, 60 bp, synthetic construct
  source 1..60: mol_type = other DNA; note = RT Primer
tcgtcggcagcgtcagatgtgtataagagacagnnnnnnnnggcccacaccgggnnnnnn

SEQ ID NO: 359 - DNA, 13 bp, synthetic construct
  source 1..13: mol_type = other DNA; note = RT Primer
gccggtagctccg

SEQ ID NO: 360 - DNA, 60 bp, synthetic construct
  source 1..60: mol_type = other DNA; note = RT Primer
tcgtcggcagcgtcagatgtgtataagagacagnnnnnnnngccggtagctccgnnnnnn

SEQ ID NO: 361 - DNA, 13 bp, synthetic construct
  source 1..13: mol_type = other DNA; note = RT Primer
gaagaattatggg

SEQ ID NO: 362 - DNA, 60 bp, synthetic construct
  source 1..60: mol_type = other DNA; note = RT Primer
tcgtcggcagcgtcagatgtgtataagagacagnnnnnnnngaagaattatgggnnnnnn

SEQ ID NO: 363 - DNA, 13 bp, synthetic construct
  source 1..13: mol_type = other DNA; note = RT Primer
gtctcgtcatatg

SEQ ID NO: 364 - DNA, 60 bp, synthetic construct
  source 1..60: mol_type = other DNA; note = RT Primer
tcgtcggcagcgtcagatgtgtataagagacagnnnnnnnngtctcgtcatatgnnnnnn

SEQ ID NO: 365 - DNA, 13 bp, synthetic construct
  source 1..13: mol_type = other DNA; note = RT Primer
ggacttttggtcg

SEQ ID NO: 366 - DNA, 60 bp, synthetic construct
  source 1..60: mol_type = other DNA; note = RT Primer
tcgtcggcagcgtcagatgtgtataagagacagnnnnnnnnggacttttggtcgnnnnnn

SEQ ID NO: 367 - DNA, 13 bp, synthetic construct
  source 1..13: mol_type = other DNA; note = RT Primer
gatttcgttccag

SEQ ID NO: 368 - DNA, 60 bp, synthetic construct
  source 1..60: mol_type = other DNA; note = RT Primer
tcgtcggcagcgtcagatgtgtataagagacagnnnnnnnngatttcgttccagnnnnnn

SEQ ID NO: 369 - DNA, 13 bp, synthetic construct
  source 1..13: mol_type = other DNA; note = RT Primer
gtcccggagccag

SEQ ID NO: 370 - DNA, 60 bp, synthetic construct
  source 1..60: mol_type = other DNA; note = RT Primer
tcgtcggcagcgtcagatgtgtataagagacagnnnnnnnngtcccggagccagnnnnnn

SEQ ID NO: 371 - DNA, 13 bp, synthetic construct
  source 1..13: mol_type = other DNA; note = RT Primer
ggggggccggacg

SEQ ID NO: 372 - DNA, 60 bp, synthetic construct
  source 1..60: mol_type = other DNA; note = RT Primer
tcgtcggcagcgtcagatgtgtataagagacagnnnnnnnnggggggccggacgnnnnnn

SEQ ID NO: 373 - DNA, 13 bp, synthetic construct
  source 1..13: mol_type = other DNA; note = RT Primer
gactacgtttggg

SEQ ID NO: 374 - DNA, 60 bp, synthetic construct
  source 1..60: mol_type = other DNA; note = RT Primer
tcgtcggcagcgtcagatgtgtataagagacagnnnnnnnngactacgtttgggnnnnnn

SEQ ID NO: 375 - DNA, 13 bp, synthetic construct
  source 1..13: mol_type = other DNA; note = RT Primer
gctttaatctaag

SEQ ID NO: 376 - DNA, 60 bp, synthetic construct
  source 1..60: mol_type = other DNA; note = RT Primer
tcgtcggcagcgtcagatgtgtataagagacagnnnnnnnngctttaatctaagnnnnnn

SEQ ID NO: 377 - DNA, 13 bp, synthetic construct
  source 1..13: mol_type = other DNA; note = RT Primer
gaggcaaaaatcg

SEQ ID NO: 378 - DNA, 60 bp, synthetic construct
  source 1..60: mol_type = other DNA; note = RT Primer
tcgtcggcagcgtcagatgtgtataagagacagnnnnnnnngaggcaaaaatcgnnnnnn

SEQ ID NO: 379 - DNA, 13 bp, synthetic construct
  source 1..13: mol_type = other DNA; note = RT Primer
gtttgattctcgg

SEQ ID NO: 380 - DNA, 60 bp, synthetic construct
  source 1..60: mol_type = other DNA; note = RT Primer
tcgtcggcagcgtcagatgtgtataagagacagnnnnnnnngtttgattctcggnnnnnn

SEQ ID NO: 381 - DNA, 13 bp, synthetic construct
  source 1..13: mol_type = other DNA; note = RT Primer
gagggacatcgag

SEQ ID NO: 382 - DNA, 60 bp, synthetic construct
  source 1..60: mol_type = other DNA; note = RT Primer
tcgtcggcagcgtcagatgtgtataagagacagnnnnnnnngagggacatcgagnnnnnn

SEQ ID NO: 383 - DNA, 13 bp, synthetic construct
  source 1..13: mol_type = other DNA; note = RT Primer
ggatccttacccg

SEQ ID NO: 384 - DNA, 60 bp, synthetic construct
  source 1..60: mol_type = other DNA; note = RT Primer
tcgtcggcagcgtcagatgtgtataagagacagnnnnnnnnggatccttacccgnnnnnn

SEQ ID NO: 385 - DNA, 13 bp, synthetic construct
  source 1..13: mol_type = other DNA; note = RT Primer
gcgatcctcgcgg

SEQ ID NO: 386 - DNA, 60 bp, synthetic construct
  source 1..60: mol_type = other DNA; note = RT Primer
tcgtcggcagcgtcagatgtgtataagagacagnnnnnnnngcgatcctcgcggnnnnnn

SEQ ID NO: 387 - DNA, 13 bp, synthetic construct
  source 1..13: mol_type = other DNA; note = RT Primer
atattggcactta

SEQ ID NO: 388 - DNA, 60 bp, synthetic construct
  source 1..60: mol_type = other DNA; note = RT Primer
tcgtcggcagcgtcagatgtgtataagagacagnnnnnnnnatattggcacttannnnnn

SEQ ID NO: 389 - DNA, 13 bp, synthetic construct
  source 1..13: mol_type = other DNA; note = RT Primer
aaaagaatcgcca

SEQ ID NO: 390 - DNA, 60 bp, synthetic construct
  source 1..60: mol_type = other DNA; note = RT Primer
tcgtcggcagcgtcagatgtgtataagagacagnnnnnnnnaaaagaatcgccannnnnn

SEQ ID NO: 391 - DNA, 13 bp, synthetic construct
  source 1..13: mol_type = other DNA; note = RT Primer
aggaagcccatta

SEQ ID NO: 392 - DNA, 60 bp, synthetic construct
  source 1..60: mol_type = other DNA; note = RT Primer
tcgtcggcagcgtcagatgtgtataagagacagnnnnnnnnaggaagcccattannnnnn

SEQ ID NO: 393 - DNA, 13 bp, synthetic construct
  source 1..13: mol_type = other DNA; note = RT Primer
accccttcgcaga

SEQ ID NO: 394 - DNA, 60 bp, synthetic construct
  source 1..60: mol_type = other DNA; note = RT Primer
tcgtcggcagcgtcagatgtgtataagagacagnnnnnnnnaccccttcgcagannnnnn

SEQ ID NO: 395 - DNA, 13 bp, synthetic construct
  source 1..13: mol_type = other DNA; note = RT Primer
atgtctattcaca

SEQ ID NO: 396 - DNA, 60 bp, synthetic construct
  source 1..60: mol_type = other DNA; note = RT Primer
tcgtcggcagcgtcagatgtgtataagagacagnnnnnnnnatgtctattcacannnnnn

SEQ ID NO: 397 - DNA, 13 bp, synthetic construct
  source 1..13: mol_type = other DNA; note = RT Primer
agaacgctttgaa

SEQ ID NO: 398 - DNA, 60 bp, synthetic construct
  source 1..60: mol_type = other DNA; note = RT Primer
tcgtcggcagcgtcagatgtgtataagagacagnnnnnnnnagaacgctttgaannnnnn

SEQ ID NO: 399 - DNA, 13 bp, synthetic construct
  source 1..13: mol_type = other DNA; note = RT Primer
aagggacccacca

SEQ ID NO: 400 - DNA, 60 bp, synthetic construct
  source 1..60: mol_type = other DNA; note = RT Primer
tcgtcggcagcgtcagatgtgtataagagacagnnnnnnnnaagggacccaccannnnnn

SEQ ID NO: 401 - DNA, 13 bp, synthetic construct
  source 1..13: mol_type = other DNA; note = RT Primer
acacatcgtttta

SEQ ID NO: 402 - DNA, 60 bp, synthetic construct
  source 1..60: mol_type = other DNA; note = RT Primer
tcgtcggcagcgtcagatgtgtataagagacagnnnnnnnnacacatcgttttannnnnn

SEQ ID NO: 403 - DNA, 13 bp, synthetic construct
  source 1..13: mol_type = other DNA; note = RT Primer
aatggttctccga

SEQ ID NO: 404 - DNA, 60 bp, synthetic construct
  source 1..60: mol_type = other DNA; note = RT Primer
tcgtcggcagcgtcagatgtgtataagagacagnnnnnnnnaatggttctccgannnnnn

SEQ ID NO: 405 - DNA, 13 bp, synthetic construct
  source 1..13: mol_type = other DNA; note = RT Primer
atgccgctgaaaa

SEQ ID NO: 406 - DNA, 60 bp, synthetic construct
  source 1..60: mol_type = other DNA; note = RT Primer
tcgtcggcagcgtcagatgtgtataagagacagnnnnnnnnatgccgctgaaaannnnnn

SEQ ID NO: 407 - DNA, 13 bp, synthetic construct
  source 1..13: mol_type = other DNA; note = RT Primer
aaacactctagta

SEQ ID NO: 408 - DNA, 60 bp, synthetic construct
  source 1..60: mol_type = other DNA; note = RT Primer
tcgtcggcagcgtcagatgtgtataagagacagnnnnnnnnaaacactctagtannnnnn

SEQ ID NO: 409 - DNA, 13 bp, synthetic construct
  source 1..13: mol_type = other DNA; note = RT Primer
atcctcggggtca

SEQ ID NO: 410 - DNA, 60 bp, synthetic construct
  source 1..60: mol_type = other DNA; note = RT Primer
tcgtcggcagcgtcagatgtgtataagagacagnnnnnnnnatcctcggggtcannnnnn

SEQ ID NO: 411 - DNA, 13 bp, synthetic construct
  source 1..13: mol_type = other DNA; note = RT Primer
aagaatcgctaga

SEQ ID NO: 412 - DNA, 60 bp, synthetic construct
  source 1..60: mol_type = other DNA; note = RT Primer
tcgtcggcagcgtcagatgtgtataagagacagnnnnnnnnaagaatcgctagannnnnn

SEQ ID NO: 413 - DNA, 13 bp, synthetic construct
  source 1..13: mol_type = other DNA; note = RT Primer
agaagtcaggtaa

SEQ ID NO: 414 - DNA, 60 bp, synthetic construct
  source 1..60: mol_type = other DNA; note = RT Primer
tcgtcggcagcgtcagatgtgtataagagacagnnnnnnnnagaagtcaggtaannnnnn

SEQ ID NO: 415 - DNA, 13 bp, synthetic construct
  source 1..13: mol_type = other DNA; note = RT Primer
acgcggctcatca

SEQ ID NO: 416 - DNA, 60 bp, synthetic construct
  source 1..60: mol_type = other DNA; note = RT Primer
tcgtcggcagcgtcagatgtgtataagagacagnnnnnnnnacgcggctcatcannnnnn

SEQ ID NO: 417 - DNA, 13 bp, synthetic construct
  source 1..13: mol_type = other DNA; note = RT Primer
aaacccaaatata

SEQ ID NO: 418 - DNA, 60 bp, synthetic construct
  source 1..60: mol_type = other DNA; note = RT Primer
tcgtcggcagcgtcagatgtgtataagagacagnnnnnnnnaaacccaaatatannnnnn

SEQ ID NO: 419 - DNA, 13 bp, synthetic construct
  source 1..13: mol_type = other DNA; note = RT Primer
atctcagaactga

SEQ ID NO: 420 - DNA, 60 bp, synthetic construct
  source 1..60: mol_type = other DNA; note = RT Primer
tcgtcggcagcgtcagatgtgtataagagacagnnnnnnnnatctcagaactgannnnnn

SEQ ID NO: 421 - DNA, 13 bp, synthetic construct
  source 1..13: mol_type = other DNA; note = RT Primer
agcaattaggaga

SEQ ID NO: 422 - DNA, 60 bp, synthetic construct
  source 1..60: mol_type = other DNA; note = RT Primer
tcgtcggcagcgtcagatgtgtataagagacagnnnnnnnnagcaattaggagannnnnn

SEQ ID NO: 423 - DNA, 13 bp, synthetic construct
  source 1..13: mol_type = other DNA; note = RT Primer
aagtaaacgcgta

SEQ ID NO: 424 - DNA, 60 bp, synthetic construct
  source 1..60: mol_type = other DNA; note = RT Primer
tcgtcggcagcgtcagatgtgtataagagacagnnnnnnnnaagtaaacgcgtannnnnn

SEQ ID NO: 425 - DNA, 13 bp, synthetic construct
  source 1..13: mol_type = other DNA; note = RT Primer
acatacagcatga

SEQ ID NO: 426 - DNA, 60 bp, synthetic construct
  source 1..60: mol_type = other DNA; note = RT Primer
tcgtcggcagcgtcagatgtgtataagagacagnnnnnnnnacatacagcatgannnnnn

SEQ ID NO: 427 - DNA, 13 bp, synthetic construct
  source 1..13: mol_type = other DNA; note = RT Primer
aacgcagagagaa

SEQ ID NO: 428 - DNA, 60 bp, synthetic construct
  source 1..60: mol_type = other DNA; note = RT Primer
tcgtcggcagcgtcagatgtgtataagagacagnnnnnnnnaacgcagagagaannnnnn

SEQ ID NO: 429 - DNA, 13 bp, synthetic construct
  source 1..13: mol_type = other DNA; note = RT Primer
atgacgcgattta

SEQ ID NO: 430 - DNA, 60 bp, synthetic construct
  source 1..60: mol_type = other DNA; note = RT Primer
tcgtcggcagcgtcagatgtgtataagagacagnnnnnnnnatgacgcgatttannnnnn

SEQ ID NO: 431 - DNA, 13 bp, synthetic construct
  source 1..13: mol_type = other DNA; note = RT Primer
aaacattcaccaa

SEQ ID NO: 432 - DNA, 60 bp, synthetic construct
  source 1..60: mol_type = other DNA; note = RT Primer
tcgtcggcagcgtcagatgtgtataagagacagnnnnnnnnaaacattcaccaannnnnn

SEQ ID NO: 433 - DNA, 13 bp, synthetic construct
  source 1..13: mol_type = other DNA; note = RT Primer
aggcctcatatta

SEQ ID NO: 434 - DNA, 60 bp, synthetic construct
  source 1..60: mol_type = other DNA; note = RT Primer
tcgtcggcagcgtcagatgtgtataagagacagnnnnnnnnaggcctcatattannnnnn

SEQ ID NO: 435 - DNA, 13 bp, synthetic construct
  source 1..13: mol_type = other DNA; note = RT Primer
tgacgctcactga

SEQ ID NO: 436 - DNA, 60 bp, synthetic construct
  source 1..60: mol_type = other DNA; note = RT Primer
tcgtcggcagcgtcagatgtgtataagagacagnnnnnnnntgacgctcactgannnnnn

SEQ ID NO: 437 - DNA, 13 bp, synthetic construct
  source 1..13: mol_type = other DNA; note = RT Primer
tccgggcggagca

SEQ ID NO: 438 - DNA, 60 bp, synthetic construct
  source 1..60: mol_type = other DNA; note = RT Primer
tcgtcggcagcgtcagatgtgtataagagacagnnnnnnnntccgggcggagcannnnnn

SEQ ID NO: 439 - DNA, 13 bp, synthetic construct
  source 1..13: mol_type = other DNA; note = RT Primer
tttcccgtttaaa

SEQ ID NO: 440 - DNA, 60 bp, synthetic construct
  source 1..60: mol_type = other DNA; note = RT Primer
tcgtcggcagcgtcagatgtgtataagagacagnnnnnnnntttcccgtttaaannnnnn

SEQ ID NO: 441 - DNA, 13 bp, synthetic construct
  source 1..13: mol_type = other DNA; note = RT Primer
tgtaactgagtga

SEQ ID NO: 442 - DNA, 60 bp, synthetic construct
  source 1..60: mol_type = other DNA; note = RT Primer
tcgtcggcagcgtcagatgtgtataagagacagnnnnnnnntgtaactgagtgannnnnn

SEQ ID NO: 443 - DNA, 13 bp, synthetic construct
  source 1..13: mol_type = other DNA; note = RT Primer
taacctatgccta

SEQ ID NO: 444 - DNA, 60 bp, synthetic construct
  source 1..60: mol_type = other DNA; note = RT Primer
tcgtcggcagcgtcagatgtgtataagagacagnnnnnnnntaacctatgcctannnnnn

SEQ ID NO: 445 - DNA, 13 bp, synthetic construct
  source 1..13: mol_type = other DNA; note = RT Primer
tccaaagaacata

SEQ ID NO: 446 - DNA, 60 bp, synthetic construct
  source 1..60: mol_type = other DNA; note = RT Primer
tcgtcggcagcgtcagatgtgtataagagacagnnnnnnnntccaaagaacatannnnnn

SEQ ID NO: 447 - DNA, 13 bp, synthetic construct
  source 1..13: mol_type = other DNA; note = RT Primer
ttctcgggtgaca

SEQ ID NO: 448 - DNA, 60 bp, synthetic construct
  source 1..60: mol_type = other DNA; note = RT Primer
tcgtcggcagcgtcagatgtgtataagagacagnnnnnnnnttctcgggtgacannnnnn

SEQ ID NO: 449 - DNA, 13 bp, synthetic construct
  source 1..13: mol_type = other DNA; note = RT Primer
tggtgttgtttta

SEQ ID NO: 450 - DNA, 60 bp, synthetic construct
  source 1..60: mol_type = other DNA; note = RT Primer
tcgtcggcagcgtcagatgtgtataagagacagnnnnnnnntggtgttgttttannnnnn

SEQ ID NO: 451 - DNA, 13 bp, synthetic construct
  source 1..13: mol_type = other DNA; note = RT Primer
taacccgtagtac

SEQ ID NO: 452 - DNA, 60 bp, synthetic construct
  source 1..60: mol_type = other DNA; note = RT Primer
tcgtcggcagcgtcagatgtgtataagagacagnnnnnnnntaacccgtagtacnnnnnn

SEQ ID NO: 453 - DNA, 13 bp, synthetic construct
  source 1..13: mol_type = other DNA; note = RT Primer
tctgcctactaac

SEQ ID NO: 454 - DNA, 60 bp, synthetic construct
  source 1..60: mol_type = other DNA; note = RT Primer
tcgtcggcagcgtcagatgtgtataagagacagnnnnnnnntctgcctactaacnnnnnn

SEQ ID NO: 455 - DNA, 13 bp, synthetic construct
  source 1..13: mol_type = other DNA; note = RT Primer
ttcagtatttatc

SEQ ID NO: 456 - DNA, 60 bp, synthetic construct
  source 1..60: mol_type = other DNA; note = RT Primer
tcgtcggcagcgtcagatgtgtataagagacagnnnnnnnnttcagtatttatcnnnnnn

SEQ ID NO: 457 - DNA, 13 bp, synthetic construct
  source 1..13: mol_type = other DNA; note = RT Primer
tgctagggaaaac

SEQ ID NO: 458 - DNA, 60 bp, synthetic construct
  source 1..60: mol_type = other DNA; note = RT Primer
tcgtcggcagcgtcagatgtgtataagagacagnnnnnnnntgctagggaaaacnnnnnn

SEQ ID NO: 459 - DNA, 13 bp, synthetic construct
  source 1..13: mol_type = other DNA; note = RT Primer
tagttacgagggc

SEQ ID NO: 460 - DNA, 60 bp, synthetic construct
  source 1..60: mol_type = other DNA; note = RT Primer
tcgtcggcagcgtcagatgtgtataagagacagnnnnnnnntagttacgagggcnnnnnn

SEQ ID NO: 461 - DNA, 13 bp, synthetic construct
  source 1..13: mol_type = other DNA; note = RT Primer
tcgacctaggttc

SEQ ID NO: 462 - DNA, 60 bp, synthetic construct
  source 1..60: mol_type = other DNA; note = RT Primer
tcgtcggcagcgtcagatgtgtataagagacagnnnnnnnntcgacctaggttcnnnnnn

SEQ ID NO: 463 - DNA, 13 bp, synthetic construct
  source 1..13: mol_type = other DNA; note = RT Primer
tgcctcgataggc

SEQ ID NO: 464 - DNA, 60 bp, synthetic construct
  source 1..60: mol_type = other DNA; note = RT Primer
tcgtcggcagcgtcagatgtgtataagagacagnnnnnnnntgcctcgataggcnnnnnn

SEQ ID NO: 465 - DNA, 13 bp, synthetic construct
  source 1..13: mol_type = other DNA; note = RT Primer
tattagtcaaacc

SEQ ID NO: 466 - DNA, 60 bp, synthetic construct
  source 1..60: mol_type = other DNA; note = RT Primer
tcgtcggcagcgtcagatgtgtataagagacagnnnnnnnntattagtcaaaccnnnnnn

SEQ ID NO: 467 - DNA, 13 bp, synthetic construct
  source 1..13: mol_type = other DNA; note = RT Primer
tcttactgaactc

SEQ ID NO: 468 - DNA, 60 bp, synthetic construct
  source 1..60: mol_type = other DNA; note = RT Primer
tcgtcggcagcgtcagatgtgtataagagacagnnnnnnnntcttactgaactcnnnnnn

SEQ ID NO: 469 - DNA, 13 bp, synthetic construct
  source 1..13: mol_type = other DNA; note = RT Primer
ttcaattgtgcac

SEQ ID NO: 470 - DNA, 60 bp, synthetic construct
  source 1..60: mol_type = other DNA; note = RT Primer
tcgtcggcagcgtcagatgtgtataagagacagnnnnnnnnttcaattgtgcacnnnnnn

SEQ ID NO: 471 - DNA, 13 bp, synthetic construct
  source 1..13: mol_type = other DNA; note = RT Primer
tgtacatgtcacc

SEQ ID NO: 472 - DNA, 60 bp, synthetic construct
  source 1..60: mol_type = other DNA; note = RT Primer
tcgtcggcagcgtcagatgtgtataagagacagnnnnnnnntgtacatgtcaccnnnnnn

SEQ ID NO: 473 - DNA, 13 bp, synthetic construct
  source 1..13: mol_type = other DNA; note = RT Primer
tacgtatcattac

SEQ ID NO: 474 - DNA, 60 bp, synthetic construct
  source 1..60: mol_type = other DNA; note = RT Primer
tcgtcggcagcgtcagatgtgtataagagacagnnnnnnnntacgtatcattacnnnnnn

SEQ ID NO: 475 - DNA, 13 bp, synthetic construct
  source 1..13: mol_type = other DNA; note = RT Primer
tcatggactctgc

SEQ ID NO: 476 - DNA, 60 bp, synthetic construct
  source 1..60: mol_type = other DNA; note = RT Primer
tcgtcggcagcgtcagatgtgtataagagacagnnnnnnnntcatggactctgcnnnnnn

SEQ ID NO: 477 - DNA, 13 bp, synthetic construct
  source 1..13: mol_type = other DNA; note = RT Primer
ttcatgctccatc

SEQ ID NO: 478 - DNA, 60 bp, synthetic construct
  source 1..60: mol_type = other DNA; note = RT Primer
tcgtcggcagcgtcagatgtgtataagagacagnnnnnnnnttcatgctccatcnnnnnn

SEQ ID NO: 479 - DNA, 13 bp, synthetic construct
  source 1..13: mol_type = other DNA; note = RT Primer
tgctaactctgcc

SEQ ID NO: 480 - DNA, 60 bp, synthetic construct
  source 1..60: mol_type = other DNA; note = RT Primer
tcgtcggcagcgtcagatgtgtataagagacagnnnnnnnntgctaactctgccnnnnnn

SEQ ID NO: 481 - DNA, 13 bp, synthetic construct
  source 1..13: mol_type = other DNA; note = RT Primer
tccctacttcaac

SEQ ID NO: 482 - DNA, 60 bp, synthetic construct
  source 1..60: mol_type = other DNA; note = RT Primer
tcgtcggcagcgtcagatgtgtataagagacagnnnnnnnntccctacttcaacnnnnnn

SEQ ID NO: 483 - DNA, 13 bp, synthetic construct
  source 1..13: mol_type = other DNA; note = RT Primer
cagtactacgcgc

SEQ ID NO: 484 - DNA, 60 bp, synthetic construct
  source 1..60: mol_type = other DNA; note = RT Primer
tcgtcggcagcgtcagatgtgtataagagacagnnnnnnnncagtactacgcgcnnnnnn

SEQ ID NO: 485 - DNA, 13 bp, synthetic construct
  source 1..13: mol_type = other DNA; note = RT Primer
ctcaccggctatc

SEQ ID NO: 486 - DNA, 60 bp, synthetic construct
  source 1..60: mol_type = other DNA; note = RT Primer
tcgtcggcagcgtcagatgtgtataagagacagnnnnnnnnctcaccggctatcnnnnnn

SEQ ID NO: 487 - DNA, 13 bp, synthetic construct
  source 1..13: mol_type = other DNA; note = RT Primer
cactatttaatac

SEQ ID NO: 488 - DNA, 60 bp, synthetic construct
  source 1..60: mol_type = other DNA; note = RT Primer
tcgtcggcagcgtcagatgtgtataagagacagnnnnnnnncactatttaatacnnnnnn

SEQ ID NO: 489 - DNA, 13 bp, synthetic construct
  source 1..13: mol_type = other DNA; note = RT Primer
ccggacttgctgc

SEQ ID NO: 490 - DNA, 60 bp, synthetic construct
  source 1..60: mol_type = other DNA; note = RT Primer
tcgtcggcagcgtcagatgtgtataagagacagnnnnnnnnccggacttgctgcnnnnnn

SEQ ID NO: 491 - DNA, 13 bp, synthetic construct
  source 1..13: mol_type = other DNA; note = RT Primer
ctcacatcttccc

SEQ ID NO: 492 - DNA, 60 bp, synthetic construct
  source 1..60: mol_type = other DNA; note = RT Primer
tcgtcggcagcgtcagatgtgtataagagacagnnnnnnnnctcacatcttcccnnnnnn

SEQ ID NO: 493 - DNA, 13 bp, synthetic construct
  source 1..13: mol_type = other DNA; note = RT Primer
cgaccacctgtac

SEQ ID NO: 494 - DNA, 60 bp, synthetic construct
  source 1..60: mol_type = other DNA; note = RT Primer
tcgtcggcagcgtcagatgtgtataagagacagnnnnnnnncgaccacctgtacnnnnnn

SEQ ID NO: 495 - DNA, 13 bp, synthetic construct
  source 1..13: mol_type = other DNA; note = RT Primer
catcaatcccagc

SEQ ID NO: 496 - DNA, 60 bp, synthetic construct
  source 1..60: mol_type = other DNA; note = RT Primer
tcgtcggcagcgtcagatgtgtataagagacagnnnnnnnncatcaatcccagcnnnnnn

SEQ ID NO: 497 - DNA, 13 bp, synthetic construct
  source 1..13: mol_type = other DNA; note = RT Primer
cccaagcaggccc

SEQ ID NO: 498 - DNA, 60 bp, synthetic construct
  source 1..60: mol_type = other DNA; note = RT Primer
tcgtcggcagcgtcagatgtgtataagagacagnnnnnnnncccaagcaggcccnnnnnn

SEQ ID NO: 499 - DNA, 13 bp, synthetic construct
  source 1..13: mol_type = other DNA; note = RT Primer
ctgtgcatagttc

SEQ ID NO: 500 - DNA, 60 bp, synthetic construct
  source 1..60: mol_type = other DNA; note = RT Primer
tcgtcggcagcgtcagatgtgtataagagacagnnnnnnnnctgtgcatagttcnnnnnn

SEQ ID NO: 501 - DNA, 13 bp, synthetic construct
  source 1..13: mol_type = other DNA; note = RT Primer
caacgcagtccac

SEQ ID NO: 502 - DNA, 60 bp, synthetic construct
  source 1..60: mol_type = other DNA; note = RT Primer
tcgtcggcagcgtcagatgtgtataagagacagnnnnnnnncaacgcagtccacnnnnnn

SEQ ID NO: 503 - DNA, 13 bp, synthetic construct
  source 1..13: mol_type = other DNA; note = RT Primer
cgcgttggtaatc

SEQ ID NO: 504 - DNA, 60 bp, synthetic construct
  source 1..60: mol_type = other DNA; note = RT Primer
tcgtcggcagcgtcagatgtgtataagagacagnnnnnnnncgcgttggtaatcnnnnnn

SEQ ID NO: 505 - DNA, 13 bp, synthetic construct
  source 1..13: mol_type = other DNA; note = RT Primer
ccttcagtaggac

SEQ ID NO: 506 - DNA, 60 bp, synthetic construct
  source 1..60: mol_type = other DNA; note = RT Primer
tcgtcggcagcgtcagatgtgtataagagacagnnnnnnnnccttcagtaggacnnnnnn

SEQ ID NO: 507 - DNA, 13 bp, synthetic construct
  source 1..13: mol_type = other DNA; note = RT Primer
ctagccttctgtc

SEQ ID NO: 508 - DNA, 60 bp, synthetic construct
  source 1..60: mol_type = other DNA; note = RT Primer
tcgtcggcagcgtcagatgtgtataagagacagnnnnnnnnctagccttctgtcnnnnnn

SEQ ID NO: 509 - DNA, 13 bp, synthetic construct
  source 1..13: mol_type = other DNA; note = RT Primer
cgtcccacgaagc

SEQ ID NO: 510 - DNA, 60 bp, synthetic construct
  source 1..60: mol_type = other DNA; note = RT Primer
tcgtcggcagcgtcagatgtgtataagagacagnnnnnnnncgtcccacgaagcnnnnnn

SEQ ID NO: 511 - DNA, 13 bp, synthetic construct
  source 1..13: mol_type = other DNA; note = RT Primer
ctagttcctattc

SEQ ID NO: 512 - DNA, 60 bp, synthetic construct
  source 1..60: mol_type = other DNA; note = RT Primer
tcgtcggcagcgtcagatgtgtataagagacagnnnnnnnnctagttcctattcnnnnnn

SEQ ID NO: 513 - DNA, 13 bp, synthetic construct
  source 1..13: mol_type = other DNA; note = RT Primer
ccccaaccggtgc

SEQ ID NO: 514 - DNA, 60 bp, synthetic construct
  source 1..60: mol_type = other DNA; note = RT Primer
tcgtcggcagcgtcagatgtgtataagagacagnnnnnnnnccccaaccggtgcnnnnnn

SEQ ID NO: 515 - DNA, 13 bp, synthetic construct
  source 1..13: mol_type = other DNA; note = RT Primer
ctcttaaagattg

SEQ ID NO: 516 - DNA, 60 bp, synthetic construct
  source 1..60: mol_type = other DNA; note = RT Primer
tcgtcggcagcgtcagatgtgtataagagacagnnnnnnnnctcttaaagattgnnnnnn

SEQ ID NO: 517 - DNA, 13 bp, synthetic construct
  source 1..13: mol_type = other DNA; note = RT Primer
cggtctccttgcg

SEQ ID NO: 518 - DNA, 60 bp, synthetic construct
  source 1..60: mol_type = other DNA; note = RT Primer
tcgtcggcagcgtcagatgtgtataagagacagnnnnnnnncggtctccttgcgnnnnnn

SEQ ID NO: 519 - DNA, 13 bp, synthetic construct
  source 1..13: mol_type = other DNA; note = RT Primer
caatgagcagagg

SEQ ID NO: 520 - DNA, 60 bp, synthetic construct
  source 1..60: mol_type = other DNA; note = RT Primer
tcgtcggcagcgtcagatgtgtataagagacagnnnnnnnncaatgagcagaggnnnnnn

SEQ ID NO: 521 - DNA, 13 bp, synthetic construct
  source 1..13: mol_type = other DNA; note = RT Primer
ccatattcccttg

SEQ ID NO: 522 - DNA, 60 bp, synthetic construct
  source 1..60: mol_type = other DNA; note = RT Primer
tcgtcggcagcgtcagatgtgtataagagacagnnnnnnnnccatattcccttgnnnnnn

SEQ ID NO: 523 - DNA, 13 bp, synthetic construct
  source 1..13: mol_type = other DNA; note = RT Primer
ctgttacccgtag

SEQ ID NO: 524 - DNA, 60 bp, synthetic construct
  source 1..60: mol_type = other DNA; note = RT Primer
tcgtcggcagcgtcagatgtgtataagagacagnnnnnnnnctgttacccgtagnnnnnn

SEQ ID NO: 525 - DNA, 13 bp, synthetic construct
  source 1..13: mol_type = other DNA; note = RT Primer
cgataattttctg

SEQ ID NO: 526 - DNA, 60 bp, synthetic construct
  source 1..60: mol_type = other DNA; note = RT Primer
tcgtcggcagcgtcagatgtgtataagagacagnnnnnnnncgataattttctgnnnnnn

SEQ ID NO: 527 - DNA, 13 bp, synthetic construct
  source 1..13: mol_type = other DNA; note = RT Primer
caaccttgagggg

SEQ ID NO: 528 - DNA, 60 bp, synthetic construct
  source 1..60: mol_type = other DNA; note = RT Primer
tcgtcggcagcgtcagatgtgtataagagacagnnnnnnnncaaccttgaggggnnnnnn

SEQ ID NO: 529 - DNA, 13 bp, synthetic construct
  source 1..13: mol_type = other DNA; note = RT Primer
ccgataaaacgcg

SEQ ID NO: 530 - DNA, 60 bp, synthetic construct
  source 1..60: mol_type = other DNA; note = RT Primer
tcgtcggcagcgtcagatgtgtataagagacagnnnnnnnnccgataaaacgcgnnnnnn

SEQ ID NO: 531 - DNA, 13 bp, synthetic construct
  source 1..13: mol_type = other DNA; note = RT Primer
gcctagagagttg

SEQ ID NO: 532 - DNA, 60 bp, synthetic construct
  source 1..60: mol_type = other DNA; note = RT Primer
tcgtcggcagcgtcagatgtgtataagagacagnnnnnnnngcctagagagttgnnnnnn

SEQ ID NO: 533 - DNA, 13 bp, synthetic construct
  source 1..13: mol_type = other DNA; note = RT Primer
ggtcaccatggag

SEQ ID NO: 534 - DNA, 60 bp, synthetic construct
  source 1..60: mol_type = other DNA; note = RT Primer
tcgtcggcagcgtcagatgtgtataagagacagnnnnnnnnggtcaccatggagnnnnnn

SEQ ID NO: 535 - DNA, 13 bp, synthetic construct
  source 1..13: mol_type = other DNA; note = RT Primer
gctgtggccccgg

SEQ ID NO: 536 - DNA, 60 bp, synthetic construct
  source 1..60: mol_type = other DNA; note = RT Primer
tcgtcggcagcgtcagatgtgtataagagacagnnnnnnnngctgtggccccggnnnnnn

SEQ ID NO: 537 - DNA, 13 bp, synthetic construct
  source 1..13: mol_type = other DNA; note = RT Primer
gtgggaggcgaag

SEQ ID NO: 538 - DNA, 60 bp, synthetic construct
  source 1..60: mol_type = other DNA; note = RT Primer
tcgtcggcagcgtcagatgtgtataagagacagnnnnnnnngtgggaggcgaagnnnnnn

SEQ ID NO: 539 - DNA, 13 bp, synthetic construct
  source 1..13: mol_type = other DNA; note = RT Primer
ggctgccgtcacg

SEQ ID NO: 540 - DNA, 60 bp, synthetic construct
  source 1..60: mol_type = other DNA; note = RT Primer
tcgtcggcagcgtcagatgtgtataagagacagnnnnnnnnggctgccgtcacgnnnnnn

SEQ ID NO: 541 - DNA, 13 bp, synthetic construct
  source 1..13: mol_type = other DNA; note = RT Primer
gatcttctatgtg

SEQ ID NO: 542 - DNA, 60 bp, synthetic construct
  source 1..60: mol_type = other DNA; note = RT Primer
tcgtcggcagcgtcagatgtgtataagagacagnnnnnnnngatcttctatgtgnnnnnn

SEQ ID NO: 543 - DNA, 13 bp, synthetic construct
  source 1..13: mol_type = other DNA; note = RT Primer
gctacttgctctg

SEQ ID NO: 544 - DNA, 60 bp, synthetic construct
  source 1..60: mol_type = other DNA; note = RT Primer
tcgtcggcagcgtcagatgtgtataagagacagnnnnnnnngctacttgctctgnnnnnn

SEQ ID NO: 545 - DNA, 13 bp, synthetic construct
  source 1..13: mol_type = other DNA; note = RT Primer
gtgggcctttagg

SEQ ID NO: 546 - DNA, 60 bp, synthetic construct
  source 1..60: mol_type = other DNA; note = RT Primer
tcgtcggcagcgtcagatgtgtataagagacagnnnnnnnngtgggcctttaggnnnnnn

SEQ ID NO: 547 - DNA, 13 bp, synthetic construct
  source 1..13: mol_type = other DNA; note = RT Primer
ggatacaccggcg

SEQ ID NO: 548 - DNA, 60 bp, synthetic construct
  source 1..60: mol_type = other DNA; note = RT Primer
tcgtcggcagcgtcagatgtgtataagagacagnnnnnnnnggatacaccggcgnnnnnn

SEQ ID NO: 549 - DNA, 13 bp, synthetic construct
  source 1..13: mol_type = other DNA; note = RT Primer
gtgcgctcggatg

SEQ ID NO: 550 - DNA, 60 bp, synthetic construct
  source 1..60: mol_type = other DNA; note = RT Primer
tcgtcggcagcgtcagatgtgtataagagacagnnnnnnnngtgcgctcggatgnnnnnn

SEQ ID NO: 551 - DNA, 13 bp, synthetic construct
  source 1..13: mol_type = other DNA; note = RT Primer
gcacaggtcaaag

SEQ ID NO: 552 - DNA, 60 bp, synthetic construct
  source 1..60: mol_type = other DNA; note = RT Primer
tcgtcggcagcgtcagatgtgtataagagacagnnnnnnnngcacaggtcaaagnnnnnn

SEQ ID NO: 553 - DNA, 13 bp, synthetic construct
  source 1..13: mol_type = other DNA; note = RT Primer
gtctgagtgcccg

SEQ ID NO: 554 - DNA, 60 bp, synthetic construct
  source 1..60: mol_type = other DNA; note = RT Primer
tcgtcggcagcgtcagatgtgtataagagacagnnnnnnnngtctgagtgcccgnnnnnn

SEQ ID NO: 555 - DNA, 13 bp, synthetic construct
  source 1..13: mol_type = other DNA; note = RT Primer
ggttgtgtagctg

SEQ ID NO: 556 - DNA, 60 bp, synthetic construct
  source 1..60: mol_type = other DNA; note = RT Primer
tcgtcggcagcgtcagatgtgtataagagacagnnnnnnnnggttgtgtagctgnnnnnn

SEQ ID NO: 557 - DNA, 13 bp, synthetic construct
  source 1..13: mol_type = other DNA; note = RT Primer
gatgcggggcggg

SEQ ID NO: 558 - DNA, 60 bp, synthetic construct
  source 1..60: mol_type = other DNA; note = RT Primer
tcgtcggcagcgtcagatgtgtataagagacagnnnnnnnngatgcggggcgggnnnnnn

SEQ ID NO: 559 - DNA, 13 bp, synthetic construct
  source 1..13: mol_type = other DNA; note = RT Primer
gccggtgtaaacg

SEQ ID NO: 560 - DNA, 60 bp, synthetic construct
  source 1..60: mol_type = other DNA; note = RT Primer
tcgtcggcagcgtcagatgtgtataagagacagnnnnnnnngccggtgtaaacgnnnnnn

SEQ ID NO: 561 - DNA, 13 bp, synthetic construct
  source 1..13: mol_type = other DNA; note = RT Primer
gtggcaagttcgg

SEQ ID NO: 562 - DNA, 60 bp, synthetic construct
  source 1..60: mol_type = other DNA; note = RT Primer
tcgtcggcagcgtcagatgtgtataagagacagnnnnnnnngtggcaagttcggnnnnnn

SEQ ID NO: 563 - DNA, 13 bp, synthetic construct
  source 1..13: mol_type = other DNA; note = RT Primer
ggtagaatgagtg

SEQ ID NO: 564 - DNA, 60 bp, synthetic construct
  source 1..60: mol_type = other DNA; note = RT Primer
tcgtcggcagcgtcagatgtgtataagagacagnnnnnnnnggtagaatgagtgnnnnnn

SEQ ID NO: 565 - DNA, 13 bp, synthetic construct
  source 1..13: mol_type = other DNA; note = RT Primer
gccctatggcccg

SEQ ID NO: 566 - DNA, 60 bp, synthetic construct
  source 1..60: mol_type = other DNA; note = RT Primer
tcgtcggcagcgtcagatgtgtataagagacagnnnnnnnngccctatggcccgnnnnnn

SEQ ID NO: 567 - DNA, 13 bp, synthetic construct
  source 1..13: mol_type = other DNA; note = RT Primer
gtctatcctcatg

SEQ ID NO: 568 - DNA, 60 bp, synthetic construct
  source 1..60: mol_type = other DNA; note = RT Primer
tcgtcggcagcgtcagatgtgtataagagacagnnnnnnnngtctatcctcatgnnnnnn

SEQ ID NO: 569 - DNA, 13 bp, synthetic construct
  source 1..13: mol_type = other DNA; note = RT Primer
ggagtgtacttgg

SEQ ID NO: 570 - DNA, 60 bp, synthetic construct
  source 1..60: mol_type = other DNA; note = RT Primer
tcgtcggcagcgtcagatgtgtataagagacagnnnnnnnnggagtgtacttggnnnnnn

SEQ ID NO: 571 - DNA, 13 bp, synthetic construct
  source 1..13: mol_type = other DNA; note = RT Primer
gatcgcgagtacg

SEQ ID NO: 572 - DNA, 60 bp, synthetic construct
  source 1..60: mol_type = other DNA; note = RT Primer
tcgtcggcagcgtcagatgtgtataagagacagnnnnnnnngatcgcgagtacgnnnnnn

SEQ ID NO: 573 - DNA, 13 bp, synthetic construct
  source 1..13: mol_type = other DNA; note = RT Primer
gcggtcccaagtg

SEQ ID NO: 574 - DNA, 60 bp, synthetic construct
  source 1..60: mol_type = other DNA; note = RT Primer
tcgtcggcagcgtcagatgtgtataagagacagnnnnnnnngcggtcccaagtgnnnnnn

SEQ ID NO: 575 - DNA, 13 bp, synthetic construct
  source 1..13: mol_type = other DNA; note = RT Primer
gttctgggagagg

SEQ ID NO: 576 - DNA, 60 bp, synthetic construct
  source 1..60: mol_type = other DNA; note = RT Primer
tcgtcggcagcgtcagatgtgtataagagacagnnnnnnnngttctgggagaggnnnnnn

SEQ ID NO: 577 - DNA, 13 bp, synthetic construct
  source 1..13: mol_type = other DNA; note = RT Primer
gaacttcgaaaag

SEQ ID NO: 578 - DNA, 60 bp, synthetic construct
  source 1..60: mol_type = other DNA; note = RT Primer
tcgtcggcagcgtcagatgtgtataagagacagnnnnnnnngaacttcgaaaagnnnnnn

SEQ ID NO: 579 - DNA, 13 bp, synthetic construct
  source 1..13: mol_type = other DNA; note = RT Primer
agcgtccacacaa

SEQ ID NO: 580 - DNA, 60 bp, synthetic construct
  source 1..60: mol_type = other DNA; note = RT Primer
tcgtcggcagcgtcagatgtgtataagagacagnnnnnnnnagcgtccacacaannnnnn

SEQ ID NO: 581 - DNA, 13 bp, synthetic construct
  source 1..13: mol_type = other DNA; note = RT Primer
acgaggtgccgta

SEQ ID NO: 582 - DNA, 60 bp, synthetic construct
  source 1..60: mol_type = other DNA; note = RT Primer
tcgtcggcagcgtcagatgtgtataagagacagnnnnnnnnacgaggtgccgtannnnnn

SEQ ID NO: 583 - DNA, 13 bp, synthetic construct
  source 1..13: mol_type = other DNA; note = RT Primer
atttattagacga

SEQ ID NO: 584 - DNA, 60 bp, synthetic construct
  source 1..60: mol_type = other DNA; note = RT Primer
tcgtcggcagcgtcagatgtgtataagagacagnnnnnnnnatttattagacgannnnnn

SEQ ID NO: 585 - DNA, 13 bp, synthetic construct
  source 1..13: mol_type = other DNA; note = RT Primer
agggctccgacaa

SEQ ID NO: 586 - DNA, 60 bp, synthetic construct
  source 1..60: mol_type = other DNA; note = RT Primer
tcgtcggcagcgtcagatgtgtataagagacagnnnnnnnnagggctccgacaannnnnn

SEQ ID NO: 587 - DNA, 13 bp, synthetic construct
  source 1..13: mol_type = other DNA; note = RT Primer
aaagtttatcaaa

SEQ ID NO: 588 - DNA, 60 bp, synthetic construct
  source 1..60: mol_type = other DNA; note = RT Primer
tcgtcggcagcgtcagatgtgtataagagacagnnnnnnnnaaagtttatcaaannnnnn

SEQ ID NO: 589 - DNA, 13 bp, synthetic construct
  source 1..13: mol_type = other DNA; note = RT Primer
acacgtgtgcgga

SEQ ID NO: 590 - DNA, 60 bp, synthetic construct
  source 1..60: mol_type = other DNA; note = RT Primer
tcgtcggcagcgtcagatgtgtataagagacagnnnnnnnnacacgtgtgcggannnnnn

SEQ ID NO: 591 - DNA, 13 bp, synthetic construct
  source 1..13: mol_type = other DNA; note = RT Primer
aacgtcaaggata

SEQ ID NO: 592 - DNA, 60 bp, synthetic construct
  source 1..60: mol_type = other DNA; note = RT Primer
tcgtcggcagcgtcagatgtgtataagagacagnnnnnnnnaacgtcaaggatannnnnn

SEQ ID NO: 593 - DNA, 13 bp, synthetic construct
  source 1..13: mol_type = other DNA; note = RT Primer
agtgacagctaca

SEQ ID NO: 594 - DNA, 60 bp, synthetic construct
  source 1..60: mol_type = other DNA; note = RT Primer
tcgtcggcagcgtcagatgtgtataagagacagnnnnnnnnagtgacagctacannnnnn

SEQ ID NO: 595 - DNA, 13 bp, synthetic construct
  source 1..13: mol_type = other DNA; note = RT Primer
aagctttacatga

SEQ ID NO: 596 - DNA, 60 bp, synthetic construct
  source 1..60: mol_type = other DNA; note = RT Primer
tcgtcggcagcgtcagatgtgtataagagacagnnnnnnnnaagctttacatgannnnnn

SEQ ID NO: 597 - DNA, 13 bp, synthetic construct
  source 1..13: mol_type = other DNA; note = RT Primer
atatgaacaacta

SEQ ID NO: 598 - DNA, 60 bp, synthetic construct
  source 1..60: mol_type = other DNA; note = RT Primer
tcgtcggcagcgtcagatgtgtataagagacagnnnnnnnnatatgaacaactannnnnn

SEQ ID NO: 599 - DNA, 13 bp, synthetic construct
  source 1..13: mol_type = other DNA; note = RT Primer
agtagatcgctaa

SEQ ID NO: 600 - DNA, 60 bp, synthetic construct
  source 1..60: mol_type = other DNA; note = RT Primer
tcgtcggcagcgtcagatgtgtataagagacagnnnnnnnnagtagatcgctaannnnnn

SEQ ID NO: 601 - DNA, 13 bp, synthetic construct
  source 1..13: mol_type = other DNA; note = RT Primer
aaagcgtctacca

SEQ ID NO: 602 - DNA, 60 bp, synthetic construct
  source 1..60: mol_type = other DNA; note = RT Primer
tcgtcggcagcgtcagatgtgtataagagacagnnnnnnnnaaagcgtctaccannnnnn

SEQ ID NO: 603 - DNA, 13 bp, synthetic construct
  source 1..13: mol_type = other DNA; note = RT Primer
acatttttgaaga

SEQ ID NO: 604 - DNA, 60 bp, synthetic construct
  source 1..60: mol_type = other DNA; note = RT Primer
tcgtcggcagcgtcagatgtgtataagagacagnnnnnnnnacatttttgaagannnnnn

SEQ ID NO: 605 - DNA, 13 bp, synthetic construct
  source 1..13: mol_type = other DNA; note = RT Primer
atgtagcttacca

SEQ ID NO: 606 - DNA, 60 bp, synthetic construct
  source 1..60: mol_type = other DNA; note = RT Primer
tcgtcggcagcgtcagatgtgtataagagacagnnnnnnnnatgtagcttaccannnnnn

SEQ ID NO: 607 - DNA, 13 bp, synthetic construct
  source 1..13: mol_type = other DNA; note = RT Primer
agtgagcggcaaa

SEQ ID NO: 608 - DNA, 60 bp, synthetic construct
  source 1..60: mol_type = other DNA; note = RT Primer
tcgtcggcagcgtcagatgtgtataagagacagnnnnnnnnagtgagcggcaaannnnnn

SEQ ID NO: 609 - DNA, 13 bp, synthetic construct
  source 1..13: mol_type = other DNA; note = RT Primer
aatgagcctgata

SEQ ID NO: 610 - DNA, 60 bp, synthetic construct
  source 1..60: mol_type = other DNA; note = RT Primer
tcgtcggcagcgtcagatgtgtataagagacagnnnnnnnnaatgagcctgatannnnnn

SEQ ID NO: 611 - DNA, 13 bp, synthetic construct
  source 1..13: mol_type = other DNA; note = RT Primer
atcgaaacgagga

SEQ ID NO: 612 - DNA, 60 bp, synthetic construct
  source 1..60: mol_type = other DNA; note = RT Primer
tcgtcggcagcgtcagatgtgtataagagacagnnnnnnnnatcgaaacgaggannnnnn

SEQ ID NO: 613 - DNA, 13 bp, synthetic construct
  source 1..13: mol_type = other DNA; note = RT Primer
agacagtagtcca

SEQ ID NO: 614 - DNA, 60 bp, synthetic construct
  source 1..60: mol_type = other DNA; note = RT Primer
tcgtcggcagcgtcagatgtgtataagagacagnnnnnnnnagacagtagtccannnnnn

SEQ ID NO: 615 - DNA, 13 bp, synthetic construct
  source 1..13: mol_type = other DNA; note = RT Primer
atttagctcggta

SEQ ID NO: 616 - DNA, 60 bp, synthetic construct
  source 1..60: mol_type = other DNA; note = RT Primer
tcgtcggcagcgtcagatgtgtataagagacagnnnnnnnnatttagctcggtannnnnn

SEQ ID NO: 617 - DNA, 13 bp, synthetic construct
  source 1..13: mol_type = other DNA; note = RT Primer
agcttgaggacca

SEQ ID NO: 618 - DNA, 60 bp, synthetic construct
  source 1..60: mol_type = other DNA; note = RT Primer
tcgtcggcagcgtcagatgtgtataagagacagnnnnnnnnagcttgaggaccannnnnn

SEQ ID NO: 619 - DNA, 13 bp, synthetic construct
  source 1..13: mol_type = other DNA; note = RT Primer
aacagacgcctaa

SEQ ID NO: 620 - DNA, 60 bp, synthetic construct
  source 1..60: mol_type = other DNA; note = RT Primer
tcgtcggcagcgtcagatgtgtataagagacagnnnnnnnnaacagacgcctaannnnnn

SEQ ID NO: 621 - DNA, 13 bp, synthetic construct
  source 1..13: mol_type = other DNA; note = RT Primer
attggacagggca

SEQ ID NO: 622 - DNA, 60 bp, synthetic construct
  source 1..60: mol_type = other DNA; note = RT Primer
tcgtcggcagcgtcagatgtgtataagagacagnnnnnnnnattggacagggcannnnnn

SEQ ID NO: 623 - DNA, 13 bp, synthetic construct
  source 1..13: mol_type = other DNA; note = RT Primer
aaaattcagtata

SEQ ID NO: 624 - DNA, 60 bp, synthetic construct
  source 1..60: mol_type = other DNA; note = RT Primer
tcgtcggcagcgtcagatgtgtataagagacagnnnnnnnnaaaattcagtatannnnnn

SEQ ID NO: 625 - DNA, 13 bp, synthetic construct
  source 1..13: mol_type = other DNA; note = RT Primer
actgtcgaaataa

SEQ ID NO: 626 - DNA, 60 bp, synthetic construct
  source 1..60: mol_type = other DNA; note = RT Primer
tcgtcggcagcgtcagatgtgtataagagacagnnnnnnnnactgtcgaaataannnnnn

SEQ ID NO: 627 - DNA, 13 bp, synthetic construct
  source 1..13: mol_type = other DNA; note = RT Primer
ttgggtatcagga

SEQ ID NO: 628 - DNA, 60 bp, synthetic construct
  source 1..60: mol_type = other DNA; note = RT Primer
tcgtcggcagcgtcagatgtgtataagagacagnnnnnnnnttgggtatcaggannnnnn

SEQ ID NO: 629 - DNA, 13 bp, synthetic construct
  source 1..13: mol_type = other DNA; note = RT Primer
tgctatcagtcta

SEQ ID NO: 630 - DNA, 60 bp, synthetic construct
  source 1..60: mol_type = other DNA; note = RT Primer
tcgtcggcagcgtcagatgtgtataagagacagnnnnnnnntgctatcagtctannnnnn

SEQ ID NO: 631 - DNA, 13 bp, synthetic construct
  source 1..13: mol_type = other DNA; note = RT Primer
taaggcggtaaga

SEQ ID NO: 632 - DNA, 60 bp, synthetic construct
  source 1..60: mol_type = other DNA; note = RT Primer
tcgtcggcagcgtcagatgtgtataagagacagnnnnnnnntaaggcggtaagannnnnn

SEQ ID NO: 633 - DNA, 13 bp, synthetic construct
  source 1..13: mol_type = other DNA; note = RT Primer
tcgattgggttta

SEQ ID NO: 634 - DNA, 60 bp, synthetic construct
  source 1..60: mol_type = other DNA; note = RT Primer
tcgtcggcagcgtcagatgtgtataagagacagnnnnnnnntcgattgggtttannnnnn

SEQ ID NO: 635 - DNA, 13 bp, synthetic construct
  source 1..13: mol_type = other DNA; note = RT Primer
ttgcccagcataa

SEQ ID NO: 636 - DNA, 60 bp, synthetic construct
  source 1..60: mol_type = other DNA; note = RT Primer
tcgtcggcagcgtcagatgtgtataagagacagnnnnnnnnttgcccagcataannnnnn

SEQ ID NO: 637 - DNA, 13 bp, synthetic construct
  source 1..13: mol_type = other DNA; note = RT Primer
tgcactgggcgca

SEQ ID NO: 638 - DNA, 60 bp, synthetic construct
  source 1..60: mol_type = other DNA; note = RT Primer
tcgtcggcagcgtcagatgtgtataagagacagnnnnnnnntgcactgggcgcannnnnn

SEQ ID NO: 639 - DNA, 13 bp, synthetic construct
  source 1..13: mol_type = other DNA; note = RT Primer
tatgggaataaaa

SEQ ID NO: 640 - DNA, 60 bp, synthetic construct
  source 1..60: mol_type = other DNA; note = RT Primer
tcgtcggcagcgtcagatgtgtataagagacagnnnnnnnntatgggaataaaannnnnn

SEQ ID NO: 641 - DNA, 13 bp, synthetic construct
  source 1..13: mol_type = other DNA; note = RT Primer
tctacccggttga

SEQ ID NO: 642 - DNA, 60 bp, synthetic construct
  source 1..60: mol_type = other DNA; note = RT Primer
tcgtcggcagcgtcagatgtgtataagagacagnnnnnnnntctacccggttgannnnnn

SEQ ID NO: 643 - DNA, 13 bp, synthetic construct
  source 1..13: mol_type = other DNA; note = RT Primer
tcatccagatcac

SEQ ID NO: 644 - DNA, 60 bp, synthetic construct
  source 1..60: mol_type = other DNA; note = RT Primer
tcgtcggcagcgtcagatgtgtataagagacagnnnnnnnntcatccagatcacnnnnnn

SEQ ID NO: 645 - DNA, 13 bp, synthetic construct
  source 1..13: mol_type = other DNA; note = RT Primer
tgaagcaattagc

SEQ ID NO: 646 - DNA, 60 bp, synthetic construct
  source 1..60: mol_type = other DNA; note = RT Primer
tcgtcggcagcgtcagatgtgtataagagacagnnnnnnnntgaagcaattagcnnnnnn

SEQ ID NO: 647 - DNA, 13 bp, synthetic construct
  source 1..13: mol_type = other DNA; note = RT Primer
tatggcgtccttc

SEQ ID NO: 648 - DNA, 60 bp, synthetic construct
  source 1..60: mol_type = other DNA; note = RT Primer
tcgtcggcagcgtcagatgtgtataagagacagnnnnnnnntatggcgtccttcnnnnnn

SEQ ID NO: 649 - DNA, 13 bp, synthetic construct
  source 1..13: mol_type = other DNA; note = RT Primer
tcaatatattgac

SEQ ID NO: 650 - DNA, 60 bp, synthetic construct
  source 1..60: mol_type = other DNA; note = RT Primer
tcgtcggcagcgtcagatgtgtataagagacagnnnnnnnntcaatatattgacnnnnnn

SEQ ID NO: 651 - DNA, 13 bp, synthetic construct
  source 1..13: mol_type = other DNA; note = RT Primer
tttggacgaccgc

SEQ ID NO: 652 - DNA, 60 bp, synthetic construct
  source 1..60: mol_type = other DNA; note = RT Primer
tcgtcggcagcgtcagatgtgtataagagacagnnnnnnnntttggacgaccgcnnnnnn

SEQ ID NO: 653 - DNA, 13 bp, synthetic construct
  source 1..13: mol_type = other DNA; note = RT Primer
tgattgaccaaac

SEQ ID NO: 654 - DNA, 60 bp, synthetic construct
  source 1..60: mol_type = other DNA; note = RT Primer
tcgtcggcagcgtcagatgtgtataagagacagnnnnnnnntgattgaccaaacnnnnnn

SEQ ID NO: 655 - DNA, 13 bp, synthetic construct
  source 1..13: mol_type = other DNA; note = RT Primer
tcgaacatacctc

SEQ ID NO: 656 - DNA, 60 bp, synthetic construct
  source 1..60: mol_type = other DNA; note = RT Primer
tcgtcggcagcgtcagatgtgtataagagacagnnnnnnnntcgaacatacctcnnnnnn

SEQ ID NO: 657 - DNA, 13 bp, synthetic construct
  source 1..13: mol_type = other DNA; note = RT Primer
tttcatccaatac

SEQ ID NO: 658 - DNA, 60 bp, synthetic construct
  source 1..60: mol_type = other DNA; note = RT Primer
tcgtcggcagcgtcagatgtgtataagagacagnnnnnnnntttcatccaatacnnnnnn

SEQ ID NO: 659 - DNA, 13 bp, synthetic construct
  source 1..13: mol_type = other DNA; note = RT Primer
tgcgggaaaggcc

SEQ ID NO: 660 - DNA, 60 bp, synthetic construct
  source 1..60: mol_type = other DNA; note = RT Primer
tcgtcggcagcgtcagatgtgtataagagacagnnnnnnnntgcgggaaaggccnnnnnn

SEQ ID NO: 661 - DNA, 13 bp, synthetic construct
  source 1..13: mol_type = other DNA; note = RT Primer
tcagggatcactc

SEQ ID NO: 662 - DNA, 60 bp, synthetic construct; identical to SEQ ID NO: 660
  source 1..60: mol_type = other DNA; note = RT Primer

SEQ ID NO: 663 - DNA, 13 bp, synthetic construct
  source 1..13: mol_type = other DNA; note = RT Primer
tgtgttacttccc

SEQ ID NO: 664 - DNA, 60 bp, synthetic construct
  source 1..60: mol_type = other DNA; note = RT Primer
tcgtcggcagcgtcagatgtgtataagagacagnnnnnnnntgtgttacttcccnnnnnn

SEQ ID NO: 665 - DNA, 13 bp, synthetic construct
  source 1..13: mol_type = other DNA; note = RT Primer
tacgggtgttgac

SEQ ID NO: 666 - DNA, 60 bp, synthetic construct
  source 1..60: mol_type = other DNA; note = RT Primer
tcgtcggcagcgtcagatgtgtataagagacagnnnnnnnntacgggtgttgacnnnnnn

SEQ ID NO: 667 - DNA, 13 bp, synthetic construct
  source 1..13: mol_type = other DNA; note = RT Primer
tcctgatggtagc

SEQ ID NO: 668 - DNA, 60 bp, synthetic construct
  source 1..60: mol_type = other DNA; note = RT Primer
tcgtcggcagcgtcagatgtgtataagagacagnnnnnnnntcctgatggtagcnnnnnn

SEQ ID NO: 669 - DNA, 13 bp, synthetic construct
  source 1..13: mol_type = other DNA; note = RT Primer
tttcgtgctttcc

SEQ ID NO: 670 - DNA, 60 bp, synthetic construct
  source 1..60: mol_type = other DNA; note = RT Primer
tcgtcggcagcgtcagatgtgtataagagacagnnnnnnnntttcgtgctttccnnnnnn

SEQ ID NO: 671 - DNA, 13 bp, synthetic construct
  source 1..13: mol_type = other DNA; note = RT Primer
tgacgcgacgatc

SEQ ID NO: 672 - DNA, 60 bp, synthetic construct
  source 1..60: mol_type = other DNA; note = RT Primer
tcgtcggcagcgtcagatgtgtataagagacagnnnnnnnntgacgcgacgatcnnnnnn

SEQ ID NO: 673 - DNA, 13 bp, synthetic construct
  source 1..13: mol_type = other DNA; note = RT Primer
tattgttaacgac

SEQ ID NO: 674 - DNA, 60 bp, synthetic construct
  source 1..60: mol_type = other DNA; note = RT Primer
tcgtcggcagcgtcagatgtgtataagagacagnnnnnnnntattgttaacgacnnnnnn

SEQ ID NO: 675 - DNA, 13 bp, synthetic construct
  source 1..13: mol_type = other DNA; note = RT Primer
ccaagcgcgtccc

SEQ ID NO: 676 - DNA, 60 bp, synthetic construct
  source 1..60: mol_type = other DNA; note = RT Primer
tcgtcggcagcgtcagatgtgtataagagacagnnnnnnnnccaagcgcgtcccnnnnnn

SEQ ID NO: 677 - DNA, 13 bp, synthetic construct
  source 1..13: mol_type = other DNA; note = RT Primer
cttatcgtcaagc

SEQ ID NO: 678 - DNA, 60 bp, synthetic construct
  source 1..60: mol_type = other DNA; note = RT Primer
tcgtcggcagcgtcagatgtgtataagagacagnnnnnnnncttatcgtcaagcnnnnnn

SEQ ID NO: 679 - DNA, 13 bp, synthetic construct
  source 1..13: mol_type = other DNA; note = RT Primer
cctagaagtcgac

SEQ ID NO: 680 - DNA, 60 bp, synthetic construct
  source 1..60: mol_type = other DNA; note = RT Primer
tcgtcggcagcgtcagatgtgtataagagacagnnnnnnnncctagaagtcgacnnnnnn

SEQ ID NO: 681 - DNA, 13 bp, synthetic construct
  source 1..13: mol_type = other DNA; note = RT Primer
ctcggcatgatcc

SEQ ID NO: 682 - DNA, 60 bp, synthetic construct
  source 1..60: mol_type = other DNA; note = RT Primer
tcgtcggcagcgtcagatgtgtataagagacagnnnnnnnnctcggcatgatccnnnnnn

SEQ ID NO: 683 - DNA, 13 bp, synthetic construct
  source 1..13: mol_type = other DNA; note = RT Primer
cgacggcgcacac

SEQ ID NO: 684 - DNA, 60 bp, synthetic construct
  source 1..60: mol_type = other DNA; note = RT Primer
tcgtcggcagcgtcagatgtgtataagagacagnnnnnnnncgacggcgcacacnnnnnn

SEQ ID NO: 685 - DNA, 13 bp, synthetic construct
  source 1..13: mol_type = other DNA; note = RT Primer
cagaccgtggacc

SEQ ID NO: 686 - DNA, 60 bp, synthetic construct
  source 1..60: mol_type = other DNA; note = RT Primer
tcgtcggcagcgtcagatgtgtataagagacagnnnnnnnncagaccgtggaccnnnnnn

SEQ ID NO: 687 - DNA, 13 bp, synthetic construct
  source 1..13: mol_type = other DNA; note = RT Primer
ccaggtgacggcc

SEQ ID NO: 688 - DNA, 60 bp, synthetic construct
  source 1..60: mol_type = other DNA; note = RT Primer
tcgtcggcagcgtcagatgtgtataagagacagnnnnnnnnccaggtgacggccnnnnnn

SEQ ID NO: 689 - DNA, 13 bp, synthetic construct
  source 1..13: mol_type = other DNA; note = RT Primer
ctcgtgtcggagc

SEQ ID NO: 690 - DNA, 60 bp, synthetic construct
  source 1..60: mol_type = other DNA; note = RT Primer
tcgtcggcagcgtcagatgtgtataagagacagnnnnnnnnctcgtgtcggagcnnnnnn

SEQ ID NO: 691 - DNA, 13 bp, synthetic construct
  source 1..13: mol_type = other DNA; note = RT Primer
ctttcgtgtagac

SEQ ID NO: 692 - DNA, 60 bp, synthetic construct
  source 1..60: mol_type = other DNA; note = RT Primer
tcgtcggcagcgtcagatgtgtataagagacagnnnnnnnnctttcgtgtagacnnnnnn

SEQ ID NO: 693 - DNA, 13 bp, synthetic construct
  source 1..13: mol_type = other DNA; note = RT Primer
cgccagtgctttc

SEQ ID NO: 694 - DNA, 60 bp, synthetic construct
  source 1..60: mol_type = other DNA; note = RT Primer
tcgtcggcagcgtcagatgtgtataagagacagnnnnnnnncgccagtgctttcnnnnnn

SEQ ID NO: 695 - DNA, 13 bp, synthetic construct
  source 1..13: mol_type = other DNA; note = RT Primer
catgttgcgggac

SEQ ID NO: 696 - DNA, 60 bp, synthetic construct
  source 1..60: mol_type = other DNA; note = RT Primer
tcgtcggcagcgtcagatgtgtataagagacagnnnnnnnncatgttgcgggacnnnnnn

SEQ ID NO: 697 - DNA, 13 bp, synthetic construct
  source 1..13: mol_type = other DNA; note = RT Primer
ccgcttgtatagc

SEQ ID NO: 698 - DNA, 60 bp, synthetic construct
  source 1..60: mol_type = other DNA; note = RT Primer
tcgtcggcagcgtcagatgtgtataagagacagnnnnnnnnccgcttgtatagcnnnnnn

SEQ ID NO: 699 - DNA, 13 bp, synthetic construct
  source 1..13: mol_type = other DNA; note = RT Primer
ctcgtcactggcc

SEQ ID NO: 700 - DNA, 60 bp, synthetic construct
  source 1..60: mol_type = other DNA; note = RT Primer
tcgtcggcagcgtcagatgtgtataagagacagnnnnnnnnctcgtcactggccnnnnnn

SEQ ID NO: 701 - DNA, 13 bp, synthetic construct
  source 1..13: mol_type = other DNA; note = RT Primer
cgcttttactgac

SEQ ID NO: 702 - DNA, 60 bp, synthetic construct
  source 1..60: mol_type = other DNA; note = RT Primer
tcgtcggcagcgtcagatgtgtataagagacagnnnnnnnncgcttttactgacnnnnnn

SEQ ID NO: 703 - DNA, 13 bp, synthetic construct
  source 1..13: mol_type = other DNA; note = RT Primer
cagcagggacgcc

SEQ ID NO: 704 - DNA, 60 bp, synthetic construct
  source 1..60: mol_type = other DNA; note = RT Primer
tcgtcggcagcgtcagatgtgtataagagacagnnnnnnnncagcagggacgccnnnnnn

SEQ ID NO: 705 - DNA, 13 bp, synthetic construct
  source 1..13: mol_type = other DNA; note = RT Primer
cccgctcgactac

SEQ ID NO: 706 - DNA, 60 bp, synthetic construct
  source 1..60: mol_type = other DNA; note = RT Primer
tcgtcggcagcgtcagatgtgtataagagacagnnnnnnnncccgctcgactacnnnnnn

SEQ ID NO: 707 - DNA, 13 bp, synthetic construct
  source 1..13: mol_type = other DNA; note = RT Primer
cttctgattttgg

SEQ ID NO: 708 - DNA, 60 bp, synthetic construct
  source 1..60: mol_type = other DNA; note = RT Primer
tcgtcggcagcgtcagatgtgtataagagacagnnnnnnnncttctgattttggnnnnnn

SEQ ID NO: 709 - DNA, 13 bp, synthetic construct
  source 1..13: mol_type = other DNA; note = RT Primer
cgatgtgcgtgtg

SEQ ID NO: 710 - DNA, 60 bp, synthetic construct
  source 1..60: mol_type = other DNA; note = RT Primer
tcgtcggcagcgtcagatgtgtataagagacagnnnnnnnncgatgtgcgtgtgnnnnnn

SEQ ID NO: 711 - DNA, 13 bp, synthetic construct
  source 1..13: mol_type = other DNA; note = RT Primer
caccccgctcctg

SEQ ID NO: 712 - DNA, 60 bp, synthetic construct
  source 1..60: mol_type = other DNA; note = RT Primer
tcgtcggcagcgtcagatgtgtataagagacagnnnnnnnncaccccgctcctgnnnnnn

SEQ ID NO: 713 - DNA, 13 bp, synthetic construct
  source 1..13: mol_type = other DNA; note = RT Primer
ccagattgtaacg

SEQ ID NO: 714 - DNA, 60 bp, synthetic construct
  source 1..60: mol_type = other DNA; note = RT Primer
tcgtcggcagcgtcagatgtgtataagagacagnnnnnnnnccagattgtaacgnnnnnn

SEQ ID NO: 715 - DNA, 13 bp, synthetic construct
  source 1..13: mol_type = other DNA; note = RT Primer
ctgctccaccaag

SEQ ID NO: 716 - DNA, 60 bp, synthetic construct
  source 1..60: mol_type = other DNA; note = RT Primer
tcgtcggcagcgtcagatgtgtataagagacagnnnnnnnnctgctccaccaagnnnnnn

SEQ ID NO: 717 - DNA, 13 bp, synthetic construct
  source 1..13: mol_type = other DNA; note = RT Primer
cgcgatattcggg

SEQ ID NO: 718 - DNA, 60 bp, synthetic construct
  source 1..60: mol_type = other DNA; note = RT Primer
tcgtcggcagcgtcagatgtgtataagagacagnnnnnnnncgcgatattcgggnnnnnn

SEQ ID NO: 719 - DNA, 13 bp, synthetic construct
  source 1..13: mol_type = other DNA; note = RT Primer
cacaggggcaacg

SEQ ID NO: 720 - DNA, 60 bp, synthetic construct
  source 1..60: mol_type = other DNA; note = RT Primer
tcgtcggcagcgtcagatgtgtataagagacagnnnnnnnncacaggggcaacgnnnnnn

SEQ ID NO: 721 - DNA, 13 bp, synthetic construct
  source 1..13: mol_type = other DNA; note = RT Primer
cctctgatccgtg

SEQ ID NO: 722 - DNA, 60 bp, synthetic construct
  source 1..60: mol_type = other DNA; note = RT Primer
tcgtcggcagcgtcagatgtgtataagagacagnnnnnnnncctctgatccgtgnnnnnn

SEQ ID NO: 723 - DNA, 13 bp, synthetic construct
  source 1..13: mol_type = other DNA; note = RT Primer
gtcattgacttgg

SEQ ID NO: 724 - DNA, 60 bp, synthetic construct
  source 1..60: mol_type = other DNA; note = RT Primer
tcgtcggcagcgtcagatgtgtataagagacagnnnnnnnngtcattgacttggnnnnnn

SEQ ID NO: 725 - DNA, 13 bp, synthetic construct
  source 1..13: mol_type = other DNA; note = RT Primer
gggaaatcgtacg

SEQ ID NO: 726 - DNA, 60 bp, synthetic construct
  source 1..60: mol_type = other DNA; note = RT Primer
tcgtcggcagcgtcagatgtgtataagagacagnnnnnnnngggaaatcgtacgnnnnnn

SEQ ID NO: 727 - DNA, 13 bp, synthetic construct
  source 1..13: mol_type = other DNA; note = RT Primer
gaacttactgcgg

SEQ ID NO: 728 - DNA, 60 bp, synthetic construct
  source 1..60: mol_type = other DNA; note = RT Primer
tcgtcggcagcgtcagatgtgtataagagacagnnnnnnnngaacttactgcggnnnnnn

SEQ ID NO: 729 - DNA, 13 bp, synthetic construct
  source 1..13: mol_type = other DNA; note = RT Primer
gcgacggattaag

SEQ ID NO: 730 - DNA, 60 bp, synthetic construct
  source 1..60: mol_type = other DNA; note = RT Primer
tcgtcggcagcgtcagatgtgtataagagacagnnnnnnnngcgacggattaagnnnnnn

SEQ ID NO: 731 - DNA, 13 bp, synthetic construct
  source 1..13: mol_type = other DNA; note = RT Primer
gttgcgatgtccg

SEQ ID NO: 732 - DNA, 60 bp, synthetic construct
  source 1..60: mol_type = other DNA; note = RT Primer
tcgtcggcagcgtcagatgtgtataagagacagnnnnnnnngttgcgatgtccgnnnnnn

SEQ ID NO: 733 - DNA, 13 bp, synthetic construct
  source 1..13: mol_type = other DNA; note = RT Primer
gggatcgcacagg

SEQ ID NO: 734 - DNA, 60 bp, synthetic construct
  source 1..60: mol_type = other DNA; note = RT Primer
tcgtcggcagcgtcagatgtgtataagagacagnnnnnnnngggatcgcacaggnnnnnn

SEQ ID NO: 735 - DNA, 13 bp, synthetic construct
  source 1..13: mol_type = other DNA; note = RT Primer
gaccaaattgtag

SEQ ID NO: 736 - DNA, 60 bp, synthetic construct
  source 1..60: mol_type = other DNA; note = RT Primer
tcgtcggcagcgtcagatgtgtataagagacagnnnnnnnngaccaaattgtagnnnnnn

SEQ ID NO: 737 - DNA, 13 bp, synthetic construct
  source 1..13: mol_type = other DNA; note = RT Primer
gctggtcagtttg

SEQ ID NO: 738 - DNA, 60 bp, synthetic construct
  source 1..60: mol_type = other DNA; note = RT Primer
tcgtcggcagcgtcagatgtgtataagagacagnnnnnnnngctggtcagtttgnnnnnn

SEQ ID NO: 739 - DNA, 13 bp, synthetic construct
  source 1..13: mol_type = other DNA; note = RT Primer
ggccaaggcgccg

SEQ ID NO: 740 - DNA, 60 bp, synthetic construct
  source 1..60: mol_type = other DNA; note = RT Primer
tcgtcggcagcgtcagatgtgtataagagacagnnnnnnnnggccaaggcgccgnnnnnn

SEQ ID NO: 741 - DNA, 13 bp, synthetic construct
  source 1..13: mol_type = other DNA; note = RT Primer
gatgtaagtcttg

SEQ ID NO: 742 - DNA, 60 bp, synthetic construct
  source 1..60: mol_type = other DNA; note = RT Primer
tcgtcggcagcgtcagatgtgtataagagacagnnnnnnnngatgtaagtcttgnnnnnn

SEQ ID NO: 743 - DNA, 13 bp, synthetic construct
  source 1..13: mol_type = other DNA; note = RT Primer
gggtgctccacag

SEQ ID NO: 744 - DNA, 60 bp, synthetic construct
  source 1..60: mol_type = other DNA; note = RT Primer
tcgtcggcagcgtcagatgtgtataagagacagnnnnnnnngggtgctccacagnnnnnn

SEQ ID NO: 745 - DNA, 13 bp, synthetic construct
  source 1..13: mol_type = other DNA; note = RT Primer
gctggtagaggtg

SEQ ID NO: 746 - DNA, 60 bp, synthetic construct
  source 1..60: mol_type = other DNA; note = RT Primer
tcgtcggcagcgtcagatgtgtataagagacagnnnnnnnngctggtagaggtgnnnnnn

SEQ ID NO: 747 - DNA, 13 bp, synthetic construct
  source 1..13: mol_type = other DNA; note = RT Primer
ggcagattccagg

SEQ ID NO: 748 - DNA, 60 bp, synthetic construct
  source 1..60: mol_type = other DNA; note = RT Primer
tcgtcggcagcgtcagatgtgtataagagacagnnnnnnnnggcagattccaggnnnnnn

SEQ ID NO: 749 - DNA, 13 bp, synthetic construct
  source 1..13: mol_type = other DNA; note = RT Primer
gatggttgcaatg

SEQ ID NO: 750 - DNA, 60 bp, synthetic construct
  source 1..60: mol_type = other DNA; note = RT Primer
tcgtcggcagcgtcagatgtgtataagagacagnnnnnnnngatggttgcaatgnnnnnn

SEQ ID NO: 751 - DNA, 13 bp, synthetic construct
  source 1..13: mol_type = other DNA; note = RT Primer
ggcgggtagatag

SEQ ID NO: 752 - DNA, 60 bp, synthetic construct
  source 1..60: mol_type = other DNA; note = RT Primer
tcgtcggcagcgtcagatgtgtataagagacagnnnnnnnnggcgggtagatagnnnnnn

SEQ ID NO: 753 - DNA, 13 bp, synthetic construct
  source 1..13: mol_type = other DNA; note = RT Primer
gccactcttgggg

SEQ ID NO: 754 - DNA, 60 bp, synthetic construct
  source 1..60: mol_type = other DNA; note = RT Primer
tcgtcggcagcgtcagatgtgtataagagacagnnnnnnnngccactcttggggnnnnnn

SEQ ID NO: 755 - DNA, 13 bp, synthetic construct
  source 1..13: mol_type = other DNA; note = RT Primer
ggtgaggtatgtg

SEQ ID NO: 756 - DNA, 60 bp, synthetic construct
  source 1..60: mol_type = other DNA; note = RT Primer
tcgtcggcagcgtcagatgtgtataagagacagnnnnnnnnggtgaggtatgtgnnnnnn

SEQ ID NO: 757 - DNA, 13 bp, synthetic construct
  source 1..13: mol_type = other DNA; note = RT Primer
gaacgtttatacg

SEQ ID NO: 758 - DNA, 60 bp, synthetic construct
  source 1..60: mol_type = other DNA; note = RT Primer
tcgtcggcagcgtcagatgtgtataagagacagnnnnnnnngaacgtttatacgnnnnnn

SEQ ID NO: 759 - DNA, 13 bp, synthetic construct
  source 1..13: mol_type = other DNA; note = RT Primer
ggtgccgccagcg

SEQ ID NO: 760 - DNA, 60 bp, synthetic construct
  source 1..60: mol_type = other DNA; note = RT Primer
tcgtcggcagcgtcagatgtgtataagagacagnnnnnnnnggtgccgccagcgnnnnnn

SEQ ID NO: 761 - DNA, 13 bp, synthetic construct
  source 1..13: mol_type = other DNA; note = RT Primer
gcgcgttactcag

SEQ ID NO: 762 - DNA, 60 bp, synthetic construct
  source 1..60: mol_type = other DNA; note = RT Primer
tcgtcggcagcgtcagatgtgtataagagacagnnnnnnnngcgcgttactcagnnnnnn

SEQ ID NO: 763 - DNA, 13 bp, synthetic construct
  source 1..13: mol_type = other DNA; note = RT Primer
gtgattgatggag

SEQ ID NO: 764 - DNA, 60 bp, synthetic construct
  source 1..60: mol_type = other DNA; note = RT Primer
tcgtcggcagcgtcagatgtgtataagagacagnnnnnnnngtgattgatggagnnnnnn

SEQ ID NO: 765 - DNA, 13 bp, synthetic construct
  source 1..13: mol_type = other DNA; note = RT Primer
ggttacgggccgg

SEQ ID NO: 766 - DNA, 60 bp, synthetic construct
  source 1..60: mol_type = other DNA; note = RT Primer
tcgtcggcagcgtcagatgtgtataagagacagnnnnnnnnggttacgggccggnnnnnn

SEQ ID NO: 767 - DNA, 13 bp, synthetic construct
  source 1..13: mol_type = other DNA; note = RT Primer
gaccagcatacag

SEQ ID NO: 768 - DNA, 60 bp, synthetic construct
  source 1..60: mol_type = other DNA; note = RT Primer
tcgtcggcagcgtcagatgtgtataagagacagnnnnnnnngaccagcatacagnnnnnn

SEQ ID NO: 769 - DNA, 13 bp, synthetic construct
  source 1..13: mol_type = other DNA; note = RT Primer
gcgagtttaatgg

SEQ ID NO: 770 - DNA, 60 bp, synthetic construct
  source 1..60: mol_type = other DNA; note = RT primer
tcgtcggcagcgtcagatgtgtataagagacagnnnnnnnngcgagtttaatggnnnnnn

SEQ ID NO: 771 - DNA, 39 bp, synthetic construct
  source 1..39: mol_type = other DNA; note = SMRT_dT Primer
aagcagtggtatcaacgcagagtttttttttttttttvn

SEQ ID NO: 772 - DNA, 23 bp, synthetic construct
  source 1..23: mol_type = other DNA; note = SMRT_PCR Primer
aagcagtggtatcaacgcagagt

SEQ ID NO: 773 - DNA, 22 bp, synthetic construct
  source 1..22: mol_type = other DNA; note = P5 Primer
aatgatacggcgaccaccgaga

SEQ ID NO: 774 - DNA, 34 bp, synthetic construct
  source 1..34: mol_type = other DNA; note = cspD_F Primer
actgggatccatgcagatggcggtttaatcgcat

SEQ ID NO: 775 - DNA, 34 bp, synthetic construct
  source 1..34: mol_type = other DNA; note = cspD_R
actgctcgagatgccatacttcgacatccttcgt

SEQ ID NO: 776 - DNA, 34 bp, synthetic construct
  source 1..34: mol_type = other DNA; note = YhcN_F Primer
actgggatccatgcaagcgttgtgatgagaacat

SEQ ID NO: 777 - DNA, 34 bp, synthetic construct
  source 1..34: mol_type = other DNA; note = YhcN_R Primer
actgctcgagatgcgatgttcacctcgtcgaatc

SEQ ID NO: 778 - DNA, 18 bp, synthetic construct
  source 1..18: mol_type = other DNA; note = RNA FISH Probe
gctgcctcccgtaggagt

SEQ ID NO: 779 - DNA, 20 bp, synthetic construct
  source 1..20: mol_type = other DNA; note = RNA FISH probe
tcgccgccgccctcagggca

SEQ ID NO: 780 - DNA, 21 bp, synthetic construct
  source 1..21: mol_type = other DNA; note = RNA FISH Probe
cgccgccgccctcagggcaga

SEQ ID NO: 781 - DNA, 20 bp, synthetic construct
  source 1..20: mol_type = other DNA; note = RNA FISH Probe
tcgccgccgccctcagggca

SEQ ID NO: 782 - DNA, 25 bp, synthetic construct
  source 1..25: mol_type = other DNA; note = RNA FISH Probe
gcgccctgcgccagcggtacatcgg

SEQ ID NO: 783 - DNA, 23 bp, synthetic construct
  source 1..23: mol_type = other DNA; note = RNA FISH Probe
gccgcccatgatcaccagccggc

SEQ ID NO: 784 - DNA, 23 bp, synthetic construct
  source 1..23: mol_type = other DNA; note = RNA FISH Probe
gcccggttggtgacgtccagccc

SEQ ID NO: 785 - DNA, 22 bp, synthetic construct
  source 1..22: mol_type = other DNA; note = RNA FISH Probe
gttccgggcgggccagccacgc

SEQ ID NO: 786 - DNA, 21 bp, synthetic construct
  source 1..21: mol_type = other DNA; note = RNA FISH Probe
cgccggctggcccagtcgtcc

SEQ ID NO: 787 - DNA, 22 bp, synthetic construct
  source 1..22: mol_type = other DNA; note = RNA FISH Probe
tcccggcgcggaccgtccagct

SEQ ID NO: 788 - DNA, 24 bp, synthetic construct
  source 1..24: mol_type = other DNA; note = RNA FISH Probe
ctccagcgtttccccgtcgcggtc

SEQ ID NO: 789 - DNA, 26 bp, synthetic construct
  source 1..26: mol_type = other DNA; note = RNA FISH Probe
cagtcacgccctgcgggagccacgtc

SEQ ID NO: 790 - DNA, 24 bp, synthetic construct
  source 1..24: mol_type = other DNA; note = RNA FISH Probe
ctggccactggccggtacgccctg

SEQ ID NO: 791 - DNA, 24 bp, synthetic construct
  source 1..24: mol_type = other DNA; note = RNA FISH Probe
ggccggttaagccgctctgcgcct

SEQ ID NO: 792 - DNA, 24 bp, synthetic construct
  source 1..24: mol_type = other DNA; note = RNA FISH Probe
ggcctggctgcaccagtaccggct

SEQ ID NO: 793 - DNA, 22 bp, synthetic construct
  source 1..22: mol_type = other DNA; note = RNA FISH Probe
gccgggccgacccacagcgtgc

(19) United States
(12) Patent Application Publication
(10) Pub. No.: US 2023/0323336 A1
(43) Pub. Date: Oct. 12, 2023

(54) HIGH-THROUGHPUT, DROPLET-BASED SINGLE CELL RNA SEQUENCING

(71) Applicants: The Broad Institute, Inc., Cambridge, MA (US); The General Hospital Corporation, Boston, MA (US)

(72) Inventors: Deborah Hung, Cambridge, MA (US); Peijun Ma, Cambridge, MA (US); Haley Amemiya, Cambridge, MA (US)

(21) Appl. No.: 17/819,034

(22) Filed: Aug. 11, 2022

Related U.S. Application Data
(60) Provisional application No. 63/231,899, filed on Aug. 11, 2021.
(60) Provisional application No. 63/341,067, filed on May 12, 2022.

(51) Int. Cl. C12N 15/10; C12Q 1/6806; C12Q 1/6869

(52) U.S. Cl. CPC C12N 15/1065; C12N 15/1096; C12Q 1/6806; C12Q 1/6869; C12Q 2563/179

(57) ABSTRACT

In one example embodiment, methods of generating a single prokaryotic cell cDNA library include barcoding RNAs from different prokaryotic cells individually, allowing cell identity of each RNA to be retained in a single sequencing library. In one example embodiment, methods include, prior to generating a cDNA library, degrading rRNA and DNA in each cell of a set of fixed and permeabilized cells. In one example embodiment, methods include converting mRNA in each fixed and permeabilized cell into cDNA labeled with a cell-specific first and second barcode combination and a unique molecular identifier (UMI). In one example embodiment, methods include amplifying labeled cDNA and preparing a sequencing library of amplified labeled cDNA. In one example embodiment, a cDNA sequencing library is capable of distinguishing species heterogeneity, strain heterogeneity, genotypic heterogeneity, phenotypic heterogeneity, or a combination thereof, of one or more uncharacterized organisms, an environmental microbiome, an organismal microbiome, or a combination thereof.

### CROSS-REFERENCE TO RELATED APPLICATIONS

This application claims the benefit of U.S. Provisional Application Nos. 63/231,899, filed Aug. 11, 2021 and 63/341,067, filed May 12, 2022. The entire contents of the above-identified applications are hereby fully incorporated herein by reference.

### STATEMENT REGARDING FEDERALLY SPONSORED RESEARCH

This invention was made with government support under Grant No. AI117043 awarded by the National Institutes of Health. The government has certain rights in the invention.

### REFERENCE TO AN ELECTRONIC SEQUENCE LISTING

The contents of the electronic sequence listing (“BROD-5365US_ST26.xml”; Size is 850,007 bytes and it was created on Aug. 9, 2022) is herein incorporated by reference in its entirety.

### TECHNICAL FIELD

The subject matter disclosed herein is generally directed to methods of creating a high-throughput droplet-based single cell RNA sequencing library for microbial cells, such as, for example, bacterial cells.

### BACKGROUND

Bacterial heterogeneity has been widely recognized in microbial communities as well as in isogeneic bacterial cultures. Besides genotypic heterogeneity, phenotypic heterogeneity also plays essential roles in bacterial survival, adaptation and evolution. Many technologies have been developed to investigate bacterial heterogeneity, including fluorescence microscopy, flow cytometry and probe-based single-cell approaches. Although these approaches have been successfully applied to single-cell microbiology, they are limited by the numbers of genes that can be studied in an experiment. In addition, genetic manipulations required to make fluorescent reporters are not only time-consuming but they may also introduce artifacts to the original strains.

### SUMMARY

In one aspect, the present invention provides for a method of generating a single prokaryotic cell cDNA library, wherein the RNAs from different prokaryotic cells are barcoded individually, allowing the cell identity of each RNA to be retained in a single library, the method comprising: a. degrading ribosomal RNA (rRNA) and genomic DNA (gDNA) in a set of fixed and permeabilized cells; b. labeling each mRNA in the fixed and permeabilized cells, the labeling comprising separating the fixed and permeabilized cells into a plurality of first reaction volumes, reverse transcribing the mRNA into cDNA, and adding a first barcode, a unique molecular identifier (UMI), and a poly-nucleotide tail to each first strand cDNA; c. labeling each cDNA from a same cell in the fixed and permeabilized cells, the labeling comprising separating the fixed and permeabilized cells into separate individual discrete second reaction volumes, conducting a second strand cDNA synthesis, and adding a second barcode unique to that second reaction volume to each second strand cDNA, thus labeling each cDNA with a cell-specific first and second barcode combination and a UMI; and d. amplifying the labeled cDNA and preparing a sequencing library comprising the amplified labeled cDNA.

In one example embodiment, the method further comprising, prior to the degrading step, preparing a set of fixed and permeabilized cells, the preparing comprising: a. fixing each cell of a set of prokaryotic cells; and b. permeabilizing each fixed cell of the set of prokaryotic cells.

In one example embodiment, each first reaction volume comprises a plurality of RT primers, wherein each RT primer comprises (i) a 5′ primer binding sequence (ii) a barcode sequence that is the same for all primers in the same first reaction volume but differs from the barcode sequence of primers in any other first reaction volume, and (iii) a unique molecular identifier (UMI) sequence that is different for each primer in a first reaction volume.

In one example embodiment, the method, each second reaction volume comprises a single bead and a plurality of primers capable of hybridizing to the poly-nucleotide tail, wherein each bead comprises a plurality of capture oligonucleotides attached 5′ to the bead surface, and wherein each capture oligonucleotide comprises (i) a barcode sequence that is the same for all capture oligonucleotides on the same bead but differs from the barcode sequence of capture oligonucleotides on other beads and (ii) a capture sequence that is the same as the 5′ primer binding sequence in each RT primer.

In one example embodiment, the generating second strand cDNA step is accomplished by linear amplification using the primers capable of hybridizing to the poly-nucleotide tail.

In one example embodiment, the second barcode is a bead barcode sequence, and wherein the adding a second barcode step is accomplished by amplifying the second strand cDNA using the capture sequence on the capture oligonucleotides attached to the bead to prime linear amplification.

In one example embodiment, mRNA accounts for more than 5%, more than 10%, more than 20%, more than 30%, more than 40%, more than 50%, more than 60%, more than 70%, or more than 80% of total aligned reads.

In one example embodiment, the mean mRNA UMI per cell is more than 25, more than 35, more than 45, more than 55, or more than 65.

In one example embodiment, each second reaction volume can be loaded with multiple fixed and permeabilized cells.

In one example embodiment, each second reaction volume comprises one, two, three, four, five, or six fixed and permeabilized cells.

In one example embodiment, each second reaction volume is an aqueous in oil droplet.

In one example embodiment, the sequencing library can distinguish species heterogeneity, strain heterogeneity, genotypic heterogeneity and/or phenotypic heterogeneity of the plurality of prokaryotic cells.

In one example embodiment, the sequencing library can distinguish species heterogeneity, strain heterogeneity, genotypic heterogeneity and/or phenotypic heterogeneity of one or more uncharacterized organisms, an environmental microbiome, or an organismal microbiome.

In one example embodiment, the sequencing library can distinguish phenotypic heterogeneity in the response of the plurality of prokaryotic cells to one or more drug treatments.

In one example embodiment, the sequencing library can distinguish phenotypic heterogeneity in the response of the plurality of prokaryotic cells to one or more antibiotic treatments.

In one example embodiment, the set of prokaryotic cells is a biological sample obtained from a subject.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, in conjunction with the accompanying drawings described herein.

FIGS. 1A-1G – BacDrop: a bacterial droplet-based high throughput scRNA-seq technology. (FIG. 1A) BacDrop workflow. Following cell fixation and permeabilization, rRNA and gDNA is depleted from cells in bulk. Then CB1 and UMIs are added to the 5′ end of cDNA via RT reactions in pools of ~200,000 cells per well in 384-well plates. After round 1 plate barcoding, all cells are pooled and cDNA is polyadenylated at the 3′ end using terminal transferase, followed by droplet generation and round 2 droplet barcoding. The 3′ poly-A tail of cDNA enables second strand synthesis using oligo-dT primers. Round 2 droplet barcoding is achieved via second strand cDNA synthesis and 4 capturing cycles by barcoded primers (on 10x gel beads) in droplets. The successfully captured cDNA contains UMIs, CB1 and CB2, as well as adaptor sequences at both 5′ and 3′ end. Each cell is identified by a combination of CB1 and CB2. (FIG. 1B) rRNA depletion inside fixed and permeabilized cells significantly increases the percentage of total reads that align to mRNA in BacDrop libraries (FIG. 1C) rRNA depletion does not affect mRNA profiles. BacDrop libraries were constructed using samples with or without rRNA depletion, and a linear regression model was fitted to the mRNA counts from each library. (FIG. 1D) Cell fixation and permeabilization does not affect mRNA profiles. Bulk RNA-seq results derived from Trizol-extracted RNA samples versus RNA derived from fixed and permeabilized cells were highly correlated. (FIG. 1E) Single-cell BacDrop results are highly correlated with bulk RNA-seq results when analyzed in bulk mode (without cell barcode extraction). (FIG. 1F) BacDrop has low barcode collision rates in an experiment where 2 million bacterial cells, mixed with K.pneumoniae and P.aeruginosa cells, were loaded into one 10x channel (~6 cells per droplet). About 2.8 % of the cells was assigned to both species. (FIG. 1G) BacDrop was performed on 4 different bacterial species including the gram-negative E.coli, K.pneumoniae, P.aeruginosa, and the gram-positive bacterium E.faecium. Uniform Manifold Approximation and Projection (UMAP) of this mixed population shows the separation of different species, colored by species identity.

FIGS. 2A-2F – Validating BacDrop’s ability to distinguish subpopulations based on distinct responses to different antibiotic treatments. (FIG. 2A), Creation of a BacDrop library containing cells of the same bacterial strain exposed to 4 different conditions. Homogeneous cultures of MGH66 were treated for 30 minutes with antibiotics: meropenem, 2 µg/mL; ciprofloxacin, 2.5 µg/mL; gentamicin, 4 µg/mL; and no antibiotic control. Cells were collected and processed separately until after round 1 plate barcoding. The four samples were then pooled for round 2 droplet barcoding and library construction. (FIG. 2B), Bulk RNA-seq results of cells exposed to the same antibiotic conditions as in a. The abundance for each treated condition and comparison between the treated and untreated cultures are shown as well as significantly up-and down- regulated genes from each treatment (performed in triplicates). (FIG. 2C) UMAP shows that BacDrop results could distinguish distinct responses from different antibiotic treatments when Applicants included ~60,000 cells with ≥ 15 mRNA detected. d-f, Applicants down-sampled 50% (FIG. 2D), 25% (FIG. 2E), and 10% (FIG. 2F) of cells from each sample and repeated the analysis as performed on the ~60,000 cells. As the number of cells included in the analysis decreases, the 4 populations arising from the different treatments becomes less distinct.

FIGS. 3A-3F – BacDrop reveals within population heterogeneity driven largely by mobile genetic elements. (FIGS. 3A, 3B), In a stable, untreated culture of MGH66, a population showing high-level expression of IS903B transposase (MGE, 5.7%; green) and a metabolically stressed population (0.25%; blue) were identified as distinct from the majority subpopulation (~94%; red). Genes expressed at significantly higher levels in these two populations are shown on the dot plot (FIG. 3B). (FIGS. 3C, 3D) Down-sampling of the data from the untreated sample to 20,000 and 10,000 cells, respectively, renders the metabolically stressed population undetectable. (FIGS. 3E, 3F) MGE-driven subpopulations were detected in another K. pneumoniae clinical isolate BIDMC35. UMAP (FIG. 3E) and heatmap (FIG. 3F) shows 4 subpopulations differing from the majority population (Cluster 0; red) of BIDMC35. Clusters 2, 3, and 4 are each driven by the high expression of a different transposase gene. In cluster 1, nearly all of the highly expressed genes belong to a prophage in BIDMC35 genome. Expression levels in all genes in FIG. 3F are normalized to expression in Cluster 0.

FIGS. 4A-4G – BacDrop reveals heterogeneous responses to meropenem exposure. (FIG. 4A) Meropenem treatment induced heterogenous subpopulations, including subpopulations showing the strongest stress response (cluster 5 and 12), undergoing active cell wall/membrane synthesis (cluster 7 and 11), undergoing active DNA replication and cell wall/membrane synthesis (cluster 9), expressing the toxin CspD (cluster 10), and highly expressing the IS903B transposase gene (MGE). (FIG. 4B) Dot plot showing the expression of genes that are significantly different among clusters and the percentage of cells expressing these genes in each cluster. (FIG. 4C) Expression of yhcN (left) and cspD (right) was only detected in their featured subpopulations. (FIG. 4D) In bulk RNA-seq results, the expression of yhcN and cspD was not significantly different in meropenem-treated versus untreated samples (black). In contrast, BacDrop revealed that the expression of yhcN was significantly higher in cluster 5 and 12, and that the expression of cspD was significantly higher in cluster 10 (gray). (FIG. 4E) Flow cytometry of reporter MGH66 strains expressing GFP driven by the promoter of yhcN (MGH66:PyhcN:gfp; left) or cspD (MGH66:PcspD:gfp; right) shows a heterogenous response to meropenem (green) but not to ciprofloxacin (blue) treatment, relative to untreated control (red). (FIG. 4F) MGH66:PcspD:gfp cells were analyzed by FACS at time 0 and 30 minutes after exposure to meropenem (2 µg/mL). After 30 minutes, live cells from GFP-low and GFP-high subpopulations were identified and sorted. (FIG. 4G) GFP-low and GFP-high subpopulations sorted from meropenem-treated MGH66:PcspD:gfp cells differed in their response to meropenem. GFP-low and GFP-high subpopulations were sorted directly into media with no antibiotic or with meropenem (2 µg/mL). Samples were then taken over time and plated on solid agar to enumerate CFU. Persisters only emerged from the GFP-high subpopulation. The limit of detection is indicated by black dashed line. Three asterisks from the treated GFP-low subpopulation indicate either no persister was present or the numbers of persisters were below the limit of detection. Three replicates were performed. The error bars were plotted as standard deviation, and two-tailed Student’s t-test were used for statistical analysis.

FIGS. 5A-5C – rRNA and gDNA depletion are implemented in BacDrop. RNA was extracted from permeabilized cells using proteinase K cell lysis (see methods). (FIG. 5A) no rRNA and gDNA depletion was performed. The three peaks are 16s rRNA (~1000 bp), 23 rRNA (~ 2000 bp), and gDNA (> 4000 bp). (FIG. 5B) only gDNA depletion was performed. (FIG. 5C) both rRNA and gDNA depletion was performed.

FIG. 6 – Scheme of two rounds of cell barcoding (plate barcoding and droplet barcoding) and library construction.

FIG. 7 – BacDrop was validated in multiples species. Unsupervised Cell clustering separate these four species into distinct clusters. Multiple subpopulations of E.faecium were identified, resulted from a small differential expression (Log2 fold change 0.6 – 0.7) of two highly expressed housekeeping genes (ef-tu and ef-g).

FIG. 8 – rRNA depletion efficiency in E.coli, P.aeruginosa, and E.faecium.

FIG. 9 – Cell barcode identification from the sequencing library containing ~ one million cells using the white list function in UMI-Tools. Cells barcodes with ≥10 UMIs per cell were selected for single-cell analysis.

FIG. 10 – Histogram of numbers of mRNA genes detected per cell in ~500,000 MGH66 cells.

FIG. 11 – Unsupervised UAMP clustering of four samples of MGH66, including the untreated, meropenem-treated, ciprofloxacin-treated, and gentamicin-treated samples. UMAP shows that BacDrop results could distinguish distinct responses from different antibiotic treatments when Applicants included ~60,000 cells with ≥ 15 mRNA detected. The ciprofloxacin-treated cells were clustered in cluster 4, and the gentamicin-treated cells were clustered in cluster 6 when 60,000 cells were included in the analysis. As the number of cells included in the analysis decreases, the four populations arising from the different treatments becomes less distinct. The MGE cluster, however, could be identified in all four samples, even when there are only 7,000 cells included in the analysis.

FIGS. 12A-12D – BacDrop results could identify sub-populations when the analysis threshold of minimal number of mRNA genes was set at 10. (FIGS. 12A, 12B) UMAP shows that BacDrop results could distinguish distinct responses from different antibiotic treatments when all ~150,000 cells were included. (FIGS. 12C, 12D) 50% down-sampling from each condition resulted in ~75,000 cells in the analysis. Reducing the numbers of cells in the analysis decreases the resolution of the separation of these four samples.

FIG. 13 – Violin plots of untreated MGH66. Numbers of mRNA genes (left) and mRNA UMIs (right) per cells were plotted. The distribution and mean numbers of mRNA genes or UMIs in three clusters were not significantly different.

FIG. 14 – The MGE subpopulation is present in all 4 samples of MGH66, including the untreated (red), and samples treated with meropenem (green), ciprofloxacin (blue), and gentamicin (purple). The numbers of cells in the MGE-driven clusters were plotted.

FIG. 15 – Violin plots of BIDMC35. Numbers of mRNA genes (left) and mRNA UMIs (right) per cells were plotted. The distribution and mean numbers of mRNA genes or UMIs in five clusters were not significantly different.

FIG. 16 – Killing kinetics of MGH66 treated with antibiotics. At each time points, cells from meropenem treatment (green), Ciprofloxacin treatment (blue), Gentamicin treatment (purple), and the untreated samples were diluted and plated on LB agar plates without antibiotics. CFUs from each condition were normalized to the CFUs of the untreated samples at the same time points to calculate the killing rates for each time points. The experiment was repeated three times and the error bars are plotted as standard deviation.

FIG. 17 – Violin plots of MGH66 treated with meropenem. Numbers of mRNA genes (left) and mRNA UMIs (right) per cells were plotted. The distribution and mean numbers of mRNA genes or UMIs in all clusters were not significantly different, except that most of cells in the MGE cluster had more mRNA genes and UMIs detected, which is caused by multiple copies of the IS903B elements. Due to limitation of the alignment program, Applicants cannot determine if the high-level expression of IS903B elements was caused by specific copies of it or all of the copies existing in the genome.

FIGS. 18A-18B – Live/Dead staining shows that the heterogeneous expression of MGH66:PcspD:gfp was not caused by cell death. (FIG. 18A), At 30 minutes of the meropenem treatment, cells were diluted into propidium iodide staining buffer (106 cells/mL) and incubated at room temperature for 15 min in the dark. The cells were then run through flowcytometry or FACS. The live cells were detected using green fluorescence and dead cells were detected using mCherry red fluorescence. In the GFP-low population, approximately 80% cells are alive, whereas in the GFP-high population, about 94% cells are alive. (FIG. 18B) Sorted cells were immediately diluted and plated on LB agar plates. CFUs were enumerated. This experiment was repeated 3 times. The error bars were plotted as standard deviation. Two-tailed Student’s t-test was used for the statistical testing (p=0.43).

FIG. 19 – Scheme of sequencing data processing.

FIG. 20 – Scheme of library structure.

FIGS. 21A-21D – Validating BacDrop’s ability to distinguish subpopulations based on distinct responses to different antibiotic treatments. (FIG. 21A) UMAP plotted based on the original identity of the 6 samples treated with meropenem (M and its replicate M.2), ciprofloxacin (C and its replicate C.2), and gentamicin (G and its replicate G.2). (FIG. 21B) Unsupervised UMAP showed three clusters with significantly (p < 0.05) higher expression of genes in the SOS-response pathway, heat-shock response, and genes encoding an IS903B transposase (MGE). (FIG. 21C) No strong batch effect was observed between the two biological replicates with the same treatment conditions. 56%-72% of the ciprofloxacin-treated cells were clustered in the SOS-response cluster, and 66%-71% of the gentamicin-treated cells were clustered in the heat-shock response cluster. All 6 samples contained cells highly expressing IS903B (MGE cluster). (FIG. 21D) Expression of a representative gene from each cluster was highlighted on the UMAP.

FIGS. 22A-22B – BacDrop reveals within population heterogeneity driven largely by mobile genetic elements (MGE). (FIGS. 22A, 22B) In an untreated culture of MGH66, a population showing high-level expression of IS903B transposase (MGE, 4.5%; green) was detected.

FIGS. 23A-23H – BacDrop reveals heterogeneous responses to meropenem exposure. (FIG. 23A) Besides the subpopulation highly expressing the IS903B transposase gene (MGE), meropenem treatment induced heterogenous responses, including a stress-response subpopulation, a cell wall synthesis subpopulation, a DNA replication and cell wall synthesis subpopulation, and a cspD-expressing subpopulation. (FIG. 23B) Dot plot showing the expression of genes that are significantly different among clusters and the percentage of cells expressing these genes in each cluster. (FIG. 23C) Validation of subpopulations identified in the meropenem-treated MGH66 using RNA FISH with double marker genes from the “cspD expressing” subpopulation (cspD + rihC) and the “strong stress response” subpopulation (rseB + yidC). A 16s rRNA probe was used as the positive control to show that more than 99% cells were successfully permeabilizated and hybridized. Subpopulations co-expressing double marker genes were identified. (FIG. 23D) Across 20 fields of view, the RNA FISH results showed that ~0.85% cells co-expressed cspD and rihC, and ~12% cells co-expressed rseB + yidC. This result verified the BacDrop result in which ~0.7% cells co-expressed cspD and rihC, and ~8% cells co-expressed rseB + yidC. (FIG. 23E) Flow cytometry of reporter MGH66 strains expressing GFP driven by the promoter of yhcN (MGH66:PyhcN:gfp; left) or cspD (MGH66:PcspD:gfp; right) shows a heterogenous response to meropenem (green) but not to ciprofloxacin (blue) treatment, relative to untreated control (red). (FIG. 23F) MGH66:PcspD:gfp cells were analyzed by FACS at time 0 and 30 minutes after exposure to meropenem (2 µg/mL). After 30 minutes, live cells from GFP-low and GFP-high subpopulations were identified and sorted. (FIG. 23G) GFP-low and GFP-high subpopulations sorted from meropenem-treated MGH66:PcspD:gfp cells differed in their response to meropenem. GFP-low and GFP-high subpopulations were sorted directly into media with no antibiotic or with meropenem (2 µg/mL). Samples were then taken over time and plated on solid agar to enumerate CFU. Persisters only emerged from the GFP-high subpopulation. The limit of detection is indicated by black dashed line. Three asterisks from the treated GFP-low subpopulation indicate either no persister was present or the numbers of persisters were below the limit of detection. (FIG. 23H) Overexpression of cspD in MGH66 increased numbers of persisters but did not affect the susceptibility of meropenem. cspD driven by the arabinose inducible promoter pBAD was transformed into MGH66. Induction of cspD with 1% arabinose did not affect the minimal inhibitory concentration (MIC) of meropenem. When cspD was induced with 1% arabinose and treated with meropenem at 2 µg/mL (purple), a greater number of persisters were formed compared to the culture without arabinose induction under 2 µg/mL meropenem treatment (blue). All experiments in panel c-h were repeated three times. Error bars were plotted as the standard deviation. The Student’s t-test was used for statistical analysis.

FIG. 24 – Development of BacDrop. RNA species and their percentage in a BacDrop library of K.pneumoniae.

FIGS. 25A-25C – BacDrop was validated in multiples species. (FIG. 25A) rRNA depletion efficiency in K.pneumoniae, P.aeruginosa, E.coli, and E.faecium. Due to the capacity of the rRNA depletion kit (10 µg total RNA maximal), the rRNA depletion efficiency is a function of cell numbers. In BacDrop protocol, Applicants used 4 × 10⁷ cell for each rRNA depletion reaction, resulting in a ~80% depletion efficiency. (FIGS. 25B, 25C) Cell lose in the mixed-species experiment is due to different cell recovery rates from centrifugation steps. In this experiment, equal numbers of cells from each species were mixed before round 1 plate barcoding (RT). Due to different cell sizes, these four species had different recovered rates at each centrifugation step (FIG. 25B) at 7,000 g. After PolyA tailing (right before round 2 droplet barcoding), the cells recovered from each species differed significantly (FIG. 25C). Therefore, Applicants recommend users to optimize centrifugation speed for different species. In cases multiplexing is needed, Applicants recommend keep samples separate until round 2 droplet barcoding. Experiments in b-d were repeated three times. Error bars are plotted as standard deviation.

FIGS. 26A-26C – Assessment of the transcriptome coverage of BacDrop libraries. (FIG. 26A) A library containing 10,000 cells of E.coli sequenced at 80,000 reads per cell. Roughly ~4,000 cells were recovered with an average of 90 mRNA genes detected per cell. (FIG. 26B) A library containing 12,000 cells of K.pneumoniae sequenced at 80,000 reads per cell. Roughly ~6,000 cells were recovered with an average of 88 mRNA genes detected per cell. (FIG. 26C) A library containing ~1 million cells of K.pneumoniae sequenced at 5,000 reads per cell. Top 3,000 cells were analyzed with an average of 127 mRNA genes detected per cell. For libraries in a and b, sequencing reads were randomly subsampled to ~40,000, ~20,000, ~4,000 reads per cell, and the analysis was repeated to calculate numbers of mRNA genes detected per cell. For library in c, sequencing reads were randomly subsampled to ~4,000, ~3,000, ~2,000 reads per cell, and the analysis was repeated to calculate numbers of mRNA genes detected per cell.

FIGS. 27A-27D – Assessment of BacDrop’s sensitivity. BacDrop has good sensitivity to distinguish cells based on the expression level of gfp. The expression levels of gfp in three E. coli strains were confirmed using flow cytometry (FIG. 27A) and the mRNA copy numbers of gfp in the sorted population were estimated using RT-qPCR (FIG. 27B). The mRNA copy numbers were 1-5 for the gfp.low strain, 9-30 for the gfp.mid strain, and 30-70 for the gfp.high strain. Roughly 3,300 cells from each strain were mixed to create a heterogeneous population and a BacDrop library was generated. (FIG. 27C) BacDrop results of gfp expression in three strains. (FIG. 27D) Identify of cells and their gfp expression levels.

FIGS. 28A-28C – Analysis of MGH66 experiment including the antibiotic-treated samples and untreated samples. (FIG. 28A) UMAP plotted based on the original identity of these 8 samples. Cells were separated well based on their treatment. (FIG. 28B) Unsupervised UMAP showed three clusters with significantly (p < 0.05) higher expression of genes in the SOS-response pathway, heat-shock response, and genes encoding an IS903B transposase (MGE). (FIG. 28C) No strong batch effect was observed between the two biological replicates with the same treatment conditions. 56%-72% of the ciprofloxacin-treated cells were clustered in the SOS-response cluster, and 66%-71% of the gentamicin-treated cells were clustered in the heat-shock response cluster. All 8 samples contained cells highly expressing IS903B (MGE cluster).

FIG. 29 – Analysis of the untreated samples. UMAP plotted based on the original identity of these two samples (replicate 1 and replicate 2).

FIG. 30 – A large number of cells is beneficial for the detection of small population. Both libraries of Replicate 1 and Replicate 2 were constructed with ~250k untreated MGH66 cells. Applicants sequenced Replicate 1 with ~5,000 reads/cell and Replicate 2 with ~3,000 reads/cell, and recovered ~40k cells and ~10k cells from Replicate 1 and Replicate 2, respectively. In these two libraries, a subpopulation highly expressing maltose transport genes (i.e, lamB) were detected. But this subpopulation was not detected in the library containing only ~3,000 untreated MGH66 cells that were collected at the same condition. The MGE subpopulation was detected in all libraries.

FIGS. 31A-31C – (FIG. 31A) Analysis of the meropenem-treated samples. UMAP plotted based on the original identity of these two samples (replicate 1 and replicate 2). (FIGS. 31B, 31C) FACS sorting coupled with Live/Dead staining to validate the expression of cspD in MGH66: PcspD:gfp strain. At 30 minutes of the meropenem treatment, cells were diluted into propidium iodide staining buffer (10⁶ cells/mL) and incubated at room temperature for 15 min in the dark. Then the cells were run through flowcytometry or subject to FACS sorting. (FIG. 31B) Gating for cells to exclude cell debris. (FIG. 31C) Dead cells were detected using mCherry red fluorescence, and GFP fluorescence of all cells was detected using green fluorescence. Roughly ~15.2% cells are dead in the gated population. The population showing no mCherry fluorescence is live cells. The gating of GFP-low and GFP-high population is shown.

FIG. 32 – Assess potential bias introduced by cell drop-off caused by insufficient sequencing depth. Since many cells were filtered out during the data analysis process due to low numbers of UMI, Applicants assessed the potential bias in RNA-seq results caused by cell drop-off. Applicants used the dataset from the untreated sample of K.pneumoniae MGH66 o calculate the correlation. Analyzed in bulk level, Applicants found the result from top 10k cells correlates well with the top 500k cells (R²=0.82), while the correlation between top 10k cells and traditional bulk RNA-seq has a R² at 0.61.

FIG. 33 – Genera captured by 16s rRNA DNA sequencing (left) and BacDrop (right). 127 genera were captured using BacDrop, whereas only 58 genera were captured using 16s rRNA DNA sequencing. 54 genera were captured by both methods. Each genera is also labeled with a number.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F.M. Ausubel et al. eds.); the series Methods in Enzymology (Academic Press, Inc.): PCR2: A Practical Approach (1995) (M.J. MacPherson, B.D. Hames, and G.R. Taylor eds.): Antibodies, A Laboratory Manual (1988) (Harlow and Lane, eds.): Antibodies A Laboratory Manual, 2^{nd} edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/-10% or less, +/-5% or less, +/-1% or less, and +/-0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Bacterial heterogeneity has been widely recognized in microbial communities and in isogenic bacterial populations. Besides genotypic heterogeneity, phenotypic heterogeneity also plays essential roles in bacterial survival, adaptation and evolution, often considered to be part of important bet-hedging or division of labor survival strategies^{1,2}. The recent advent of single-cell RNA-seq (scRNA-seq) has fostered numerous important discoveries in mammalian systems, resulting in a transforming appreciation of the heterogeneity in cell types and cell states^{3,4}. However, bacterial scRNA-seq is still underdeveloped due to several challenges⁵, including difficulties in bacterial lysis, ribosomal RNA (rRNA) contamination, the absence of a polyadenylated tail for messenger RNA (mRNA) capture, and the paucity of mRNA content in a single bacterial cell⁶. Recently, several bacterial scRNA-seq approaches have been developed, including the plate-based methods⁷⁻⁹, such as microSPLiT, PETRI-seq, and MATQ-seq. However, these plate-based scRNA-seq technologies have to date been limited by the numbers of cells that can be studied in one experiment and the overwhelming dominance of rRNA in the sequencing libraries. Meanwhile, recent probe-based approaches^{10,11}, such as par-seqFISH, have also been described, which differ in their ability to query the entire genome in a completely unbiased way, but with other potential advantages. Par-seqFISH, for example, has the advantage of being able to analyze large numbers of cells while excluding rRNA and providing spatial resolution of a bacterial population; however, prior definition of the genome(s) of interest is required to enable probe design and generation, with a pragmatic limitation to the numbers of genes that can be queried. Additionally, studies using these previously reported methods have mainly focused on between population heterogeneity, with a focus on proof of principle, whereas within population heterogeneity has not been extensively described.

Embodiments disclosed herein provide a droplet-based high throughput bacterial scRNA-seq technology that can be used to investigate millions of single bacterial cells simultaneously (BacDrop). No probe-design and prior knowledge of the genome is required. BacDrop can be used for many species and can be used for microbiome study. The optimized cell permeabilization method allows complete gDNA removal and efficient rRNA depletion. Two rounds of cell barcoding increase the throughput by at least 10 times in each microfluidic channel (e.g., microfluidic channels for forming droplets). The method is high throughput due to a second cell barcoding step performed in droplets. This allows for the analysis of >1,000,000 cells per sample.

The following detailed description is of example embodiments of the presently claimed invention with references to the accompanying drawings. Such description is intended to be illustrative and not limiting with respect to the scope of the present invention. Such embodiments are described in sufficient detail to enable one of ordinary skill in the art to practice the subject invention, and it will be understood that other embodiments may be practiced with some variations without departing from the spirit or scope of the subject invention.

### Methods of Generating a Single Prokaryotic Cell cDNA Library

In an aspect, the present disclosure provides methods of generating a single prokaryotic cell cDNA library. In one example embodiment, methods include barcoding RNAs from different prokaryotic cells individually, allowing cell identity of each RNA to be retained in a single sequencing library. In one example embodiment, methods include degrading rRNA and gDNA in fixed and permeabilized cells prior to generation of a cDNA library. In one example embodiment, methods include converting mRNA into cDNA and labeling cDNA with a cell-specific first and second barcode combination and a unique molecular identifier (UMI) in fixed and permeabilized cells. In one example embodiment, methods include amplifying labeled cDNA and preparing a sequencing library of amplified labeled cDNA. In one example embodiment, a cDNA sequencing library is capable of distinguishing species heterogeneity, strain heterogeneity, genotypic heterogeneity, phenotypic heterogeneity, or a combination thereof, of one or more uncharacterized organisms, an environmental microbiome, an organismal microbiome, or a combination thereof. Non-limiting examples of methods are provided herein.

In one example embodiment, methods comprise: degrading rRNA and gDNA in a set of fixed and permeabilized cells; labeling each mRNA in the fixed and permeabilized cells by reverse transcribing the mRNA into cDNA, the labeling adding a first barcode, a unique molecular identifier (UMI), and a poly-nucleotide tail to each first strand cDNA; labeling each cDNA from a same cell in the fixed and permeabilized cells, the labeling comprising separating the fixed and permeabilized cells into separate individual discrete second reaction volumes, conducting a second strand cDNA synthesis, and adding a second barcode unique to that second reaction volume to each second strand cDNA, thus labeling each cDNA with a cell-specific first and second barcode combination and a UMI; and amplifying the labeled cDNA and preparing a sequencing library comprising the amplified labeled cDNA.

### Prokaryotic Cell Samples

Samples comprising prokaryotic cells may be analyzed using the single cell sequencing methods described herein. Samples can be prokaryotic cells grown in the lab. The samples can be homogenous cultures. Samples can be microbial cells treated with an agent or perturbation (e.g., antibiotic). Samples can be isolated from a biological source, such as an animal or human subject. Samples can also be isolated from environmental sources such as soil and water. In one example embodiment, a microbiome is analyzed from a subject. Methods of obtaining a sample from a subject can use any method known in the art. In an exemplary embodiment, a sample can be obtained by a swab. In another exemplary embodiment, a sample is obtained during a surgery. Samples may be obtained from the throat, nose, rectum, or vaginal surfaces. Samples obtained may be cultured on media before analysis of single cells.

The methods may be applied to all prokaryotic cells including bacteria and archaea. In one example embodiment, the prokaryotic cells are Gram-negative or Gram-positive. Various combinations of cells may form a set of prokaryotic cells. In one example embodiment, a set of prokaryotic cells includes one or more prokaryotic cell species, one or more strains of the same prokaryotic cell species, one or more phenotypes of the same prokaryotic cell species, one or more phenotypes of the same prokaryotic cell strain, or a combination thereof. In one example embodiment, a set of prokaryotic cells is a biological sample obtained from a subject. In another example embodiment, a set of prokaryotic cells is a biological sample comprising one or more uncharacterized organisms, an environmental microbiome, or an organismal microbiome.

In various examples, the microbial cells can include one or more bacterial species selected from an Acinetobacter species, an Actinobacillus species, an Actinomycetes species, an Actinomyces species, Aerococcus species an Aeromonas species, an Anaplasma species, an Alcaligenes species, a Bacillus species, a Bacteroides species, a Bartonella species, a Bifidobacterium species, a Bordetella species, a Borrelia species, a Brucella species, a Burkholderia species, a Campylobacter species, a Capnocytophaga species, a Chlamydia species, a Citrobacter species, a Coxiella species, a Corynbacterium species, a Clostridium species, an Eikenella species, an Enterobacter species, an Escherichia species, an Enterococcus species, an Ehlichia species, an Epidermophyton species, an Erysipelothrix species, a Eubacterium species, a Francisella species, a Fusobacterium species, a Gardnerella species, a Gemella species, a Haemophilus species, a Helicobacter species, a Kingella species, a Klebsiella species, a Lactobacillus species, a Lactococcus species, a Listeria species, a Leptospira species, a Legionella species, a Leptospira species, Leuconostoc species, a Mannheimia species, a Microsporum species, a Micrococcus species, a Moraxella species, a Morganell species, a Mobiluncus species, a Micrococcus species, Mycobacterium species, a Mycoplasm species, a Nocardia species, a Neisseria species, a Pasteurelaa species, a Pediococcus species, a Peptostreptococcus species, a Pityrosporum species, a Plesiomonas species, a Prevotella species, a Porphyromonas species, a Proteus species, a Providencia species, a Pseudomonas species, a Propionibacteriums species, a Rhodococcus species, a Rickettsia species, a Rhodococcus species, a Serratia species, a Stenotrophomonas species, a Salmonella species, a Serratia species, a Shigella species, a Staphylococcus species, a Streptococcus species, a Spirillum species, a Streptobacillus species, a Treponema species, a Tropheryma species, a Trichophyton species, an Ureaplasma species, a Veillonella species, a Vibrio species, a Yersinia species, and a Xanthomonas species. In another example embodiment, the plurality of microbial cells comprise cells from one or more bacterial species selected from the group consisting of Escherichia coli, Klebsiella pneumoniae, Pseudomonas aeruginosa, and Enterococcus faecium.

### Degrading Ribosomal RNA (rRNA) and Genomic DNA (gDNA) in a Set of Fixed and Permeabilized Cells

In one example embodiment, a portion, substantially all, or all of the cells of a set of prokaryotic cells are fixed. In one example embodiment, a portion, substantially all, or all of the cells of a set of prokaryotic cells are fixed by crosslinking.

In one example embodiment, a portion, substantially all, or all of the cell wall of each fixed cell of a set of prokaryotic cells are permeabilized. In one example embodiment, a portion, substantially all, or all of the cell wall of each fixed cell of a set of prokaryotic cells are permeabilized by hydrolysis.

### Preparing Fixed and Permeabilized Cells

In one example embodiment, preparing a set of fixed and permeabilized cells is performed prior to degrading rRNA and gDNA. In one example embodiment, preparing a set of fixed and permeabilized cells comprises fixing each cell of a set of prokaryotic cells and permeabilizing each fixed cell of the set of prokaryotic cells. The fixing and permeabilizing of cells may be performed in two separate sequential steps, two partially overlapping sequential steps, or a single concurrent step.

In one example embodiment, fixing each cell of a set of prokaryotic cells comprises fixing a portion, substantially all, or all of each cell of a set of prokaryotic cells. Various fixing methods can be used. In one example embodiment, fixing is accomplished by crosslinking. Non-limiting methods of crosslinking are known in the art. Fixation methods can be divided into two groups: additive and denaturing fixation. Additive fixation solutions (also called cross-linking fixations) contain various aldehydes, including formaldehyde, paraformaldehyde, glutaraldehyde, etc., and can create covalent chemical bonds between proteins. This method can preserve the natural structure of proteins, i.e., secondary and tertiary structures. Another group is the denaturing (or precipitating) fixations. These methods can denature proteins by reducing their solubility and/or disrupting the hydrophobic interactions, and thus modify the tertiary structures of proteins as well as inactivate enzymes. Alcohols, such as methanol and ethanol, are commonly used for denaturing fixation. However, alcohols are seldom solely applied since they can induce serious cell shrinkage. Other denaturing chemicals, like acetone and acetic acid, are usually combined with alcohols to enhance the fixation performance. Common fixation solutions include 2.5% glutaraldehyde, 4-10% formalin (formalin is an alternative name for an aqueous solution of formaldehyde), 4% paraformaldehyde, methanol/acetone (1:1), and ethanol/acetic acid (3:1) (see, e.g., Chao Y, Zhang T. Optimization of fixation methods for observation of bacterial cell morphology and surface ultrastructures by atomic force microscopy. Appl Microbiol Biotechnol. 2011;92(2):381-392). In one example embodiment, a set of prokaryotic cells is fixed in a single fixing reaction volume comprising about 10 billion prokaryotic cells or less and/or about 143 million prokaryotic cells/ml or less. In one example embodiment, a set of prokaryotic cells is fixed in a plurality of fixing reaction volumes each comprising about 10 billion prokaryotic cells or less at about 143 million prokaryotic cells/ml or less. In one example embodiment, the fixing step is accomplished by addition of formaldehyde or paraformaldehyde. Fixing of bacteria may employ 4% (wt/vol) paraformaldehyde or formaldehyde, aldehydes that cross-link cellular macromolecules, ultimately creating a mesh type structure within the cell.

In one example embodiment, a permeabilizing step comprises permeabilizing a portion, substantially all, or all of the cell wall and cytoplasmic membrane of each fixed cell of a set of prokaryotic cells. The bacterial cell wall consists of peptidoglycan, an essential protective barrier for bacterial cells that encapsulates the cytoplasmic membrane of both Gram-positive and Gram-negative bacterial cells. Peptidoglycan is a rigid, highly conserved, complex structure of polymeric carbohydrates and amino acids. Various permeabilizing methods can be used. In one example embodiment, permeabilizing is accomplished by hydrolysis. Non-limiting methods of hydrolysis are known in the art. Different classes of permeabilizers include, but are not limited to organic solvents (ethanol), detergents (triton X-100) and enzymes (lysozyme). Lysozyme is a glycoside hydrolase that catalyzes the hydrolysis of 1,4-beta-linkages between N-acetylmuramic acid and N-acetyl-D-glucosamine residues in peptidoglycan, which is the major component of gram-positive bacterial cell wall. Lysozyme attacks peptidoglycans (found in the cell walls of bacteria, especially Gram-positive bacteria). In certain embodiments, the permeabilizing step comprises treatment with ethanol, detergent and/or lysozyme (see, e.g., Rocha R, Almeida C, Azevedo NF (2018) Influence of the fixing/permeabilizing step on peptide nucleic acid fluorescence in situ hybridization (PNA-FISH) for the detection of bacteria. PLoS ONE 13(5): e0196522). Gram-negative and many Gram-positive species may require the use of permeabilizing agents such as enzymes, solvents, detergents or even hydrochloric acid. Id. The choice of the permeabilizing procedure to be employed will depend on the characteristics of the microorganism(s) and their cell envelope composition. In preferred embodiments, a universal permeabilizing step applicable to all or substantially all microorganisms is used that includes treatment with ethanol, detergent and lysozyme (e.g., 50% Ethanol in one step, 0.04% Tween-20 in another step, and lysozyme in another step). In one example embodiment, a set of fixed prokaryotic cells is permeabilized in a single permeabilizing reaction volume comprising about 40 million fixed prokaryotic cells or less and/or about 200,000 fixed prokaryotic cells/µL or less. In another example embodiment, a set of fixed prokaryotic cells is permeabilized in a plurality of permeabilizing reaction volumes each comprising about 40 million fixed prokaryotic cells or less and/or about 200,000 fixed prokaryotic cells/µL or less.

### Degrading rRNA and gDNA in Fixed and Permeabilized Cells

Various methods of rRNA and gDNA degradation can be used. In one example embodiment, methods degrade rRNA and gDNA in a set of fixed and permeabilized cells. The permeabilization methods disclosed herein increase access to rRNA and gDNA resulting in increased rRNA and gDNA degradation (e.g., complete gDNA removal and efficient rRNA depletion). Non-limiting methods of removing rRNA and gDNA are known in the art (see, e.g., Giannoukos G., Ciulla D.M., Huang K., Haas B.J., Izard J., Levin J.Z., Livny J., Earl A.M., Gevers D., Ward D.V. et al. Efficient and robust RNA-seq process for cultured bacteria and complex community transcriptomes. Genome Biol. 2012; 13:R23; He S.M., Wurtzel O., Singh K., Froula J.L., Yilmaz S., Tringe S.G., Wang Z., Chen F., Lindquist E.A., Sorek R. et al. Validation of two ribosomal RNA removal methods for microbial metatranscriptomics. Nat. Methods. 2010; 7:U807-U858; Petrova O.E., Garcia-Alcalde F., Zampaloni C., Sauer K. Comparative evaluation of rRNA depletion procedures for the improved analysis of bacterial biofilm and mixed pathogen culture transcriptomes. Sci. Rep. 2017; 7:41114; He S.Izard J, Rivera MC Chapter 3 - Ribosomal RNA removal methods for microbial transcriptomics. Metagenomics for Microbiology. 2015; LondonElsevier39-53; Dunman P.M., Murphy E., Haney S., Palacios D., Tucker-Kellogg G., Wu S., Brown E.L., Zagursky R.J., Shlaes D., Projan S.J. Transcription profiling-based identification of Staphylococcus aureus genes regulated by the agr and/or sarA loci. J. Bacteriol. 2001; 183:7341-7353; Stewart F.J., Ottesen E.A., DeLong E.F. Development and quantitative analyses of a universal rRNA-subtraction protocol for microbial metatranscriptomics. ISMEJ. 2010; 4:896-907; Li S.K., Zhou J.W., Yim A.K.Y., Leung A.K.Y., Tsui S.K.W., Chan T.F., Lau T.C.K. Organism-specific rRNA capture system for application in next-generation sequencing. PLoS One. 2013; 8:e74286; Rey F.E., Faith J.J., Bain J., Muehlbauer M.J., Stevens R.D., Newgard C.B., Gordon J.I. Dissecting the in vivo metabolic potential of two human gut acetogens. J. Biol. Chem. 2010; 285:22082-22090; and Kim I.V., Ross E.J., Dietrich S., Döring K., Alvarado S.A., Kuhn C. Efficient depletion of ribosomal RNA for RNA sequencing in planarians. BMC Genomics. 2019; 20:909.

In one example embodiment, for rRNA degradation, single stranded probes that hybridize specifically to bacterial rRNA are contacted with the cells followed by contacting the cells with RNase H, which degrades the hybridized RNA (see, e.g., NEBNext Bacterial rRNA Depletion Kit, New England Biolabs, Ipswich, MA; Huang Y, Sheth RU, Kaufman A, Wang HH. Scalable and cost-effective ribonuclease-based rRNA depletion for transcriptomics. Nucleic Acids Res. 2020;48(4):e20); and Engelhardt F, Tomasch J, Häussler S. Organism-specific depletion of highly abundant RNA species from bacterial total RNA. Access Microbiol. 2020;2(10):acmi000159).

In another example embodiment, blocking primers specific to rRNA sequences are contacted with the fixed and permeabilized cells to prevent reverse transcription of rRNA (see, e.g., Wangsanuwat, et al., Efficient and cost-effective bacterial mRNA sequencing from low input samples through ribosomal RNA depletion. bioRxiv 2020.06.19.162412; and Wangsanuwat, C., Heom, K.A., Liu, E. et al. Efficient and cost-effective bacterial mRNA sequencing from low input samples through ribosomal RNA depletion. BMC Genomics 21, 717 (2020)).

In another example embodiment, CRISPR is used to deplete rRNA cDNA (i.e., after cDNA synthesis and before sequencing) (see, e.g., Prezza G, Heckel T, Dietrich S, Homberger C, Westermann AJ, Vogel J. Improved bacterial RNA-seq by Cas9-based depletion of ribosomal RNA reads. RNA. 2020;26(8):1069-1078. doi:10.1261/rna.075945.120; and CRISPRclean Pan Bacterial rRNA Depletion Kit, Jumpcode Genomics, San Diego, CA). In certain embodiments, CRISPR depletion is performed after the reverse transcription step. In certain embodiments, CRISPR depletion is performed after sequencing library preparation. In certain embodiments, rRNA is depleted by a method that removes or blocks rRNA before the reverse transcription step and a CRISPR method is used before sequencing to remove remaining rRNA sequences.

In another example embodiment, rRNA cDNA can be removed after the barcoding steps and before sequencing. For example, rRNA depletion can be performed by hybridizing samples to probes antisense to the rRNA cDNA to be depleted, where the antisense probes include a capture moiety that can be captured by a specific binding agent. In some examples, depleting the samples of non-target RNA cDNA comprises using one or more probes that specifically hybridize to the non-target RNA cDNA, wherein the one or more probes comprise a label that facilitates removal of the probe from the sample. In some examples the one or more probes is a plurality of probes, wherein probes in the plurality are selected to tile across the sequence of the non-target RNA cDNA. In some examples, the label comprises biotin. In some examples, the probes are biotinylated antisense probes that deplete ribosomal RNA cDNAs. The probe-RNA cDNA is later bound to streptavidin beads to physically remove the target RNA cDNAs from the solution. Due to the GC-rich sequence of 28S, it is better to use not only biotinylated UTP, but also biotinylated CTP.

Non-limiting methods of removing gDNA are known in the art. In example embodiments, gDNA is removed using DNase treatment. A deoxyribonuclease (DNase) is an enzyme that catalyzes the hydrolytic cleavage of phosphodiester linkages in the DNA backbone, thus degrading DNA. Deoxyribonucleases are one type of nuclease, a generic term for enzymes capable of hydrolyzing phosphodiester bonds that link nucleotides. Some DNases cut, or “cleave”, only residues at the ends of DNA molecules (exodeoxyribonucleases, a type of exonuclease). Others cleave anywhere along the chain (endodeoxyribonucleases, a subset of endonucleases). Some DNases are fairly indiscriminate about the DNA sequence at which they cut, while others, including restriction enzymes, are very sequence-specific. Some cleave only double-stranded DNA; others are specific for single-stranded molecules; and still others are active toward both. In example embodiments, a combination of DNases can be used or a single DNase can be used. In preferred embodiments, DNase I is used to degrade genomic DNA. Deoxyribonuclease I (DNase I) is an endonuclease of the DNase family coded by the human gene DNASE1. DNase I is a nuclease that cleaves DNA preferentially at phosphodiester linkages adjacent to a pyrimidine nucleotide, yielding 5′-phosphate-terminated polynucleotides with a free hydroxyl group on position 3′, on average producing tetranucleotides. DNase I acts on single-stranded DNA, double-stranded DNA, and chromatin.

In an example embodiment, rRNA and gDNA degradation may be performed in two separate sequential steps, two partially overlapping sequential steps, or a single concurrent step.

In one example embodiment, methods selectively degrade rRNA and gDNA over mRNA. In one example embodiment, mRNA in a set of fixed and permeabilized cells is not substantially degraded (e.g., degradation does not result in a detectable change in the mRNA profile).

Methods can be performed to various percent enrichment of mRNA. In one example embodiment, mRNA accounts for more than 5%, more than 10%, more than 20%, more than 30%, more than 40%, more than 50%, more than 60%, more than 70%, or more than 80% of total aligned reads in a cDNA library. In certain aspects, the mean mRNA UMI per cell is more than 25, more than 35, more than 45, more than 55, or more than 65.

In one example embodiment, a set of fixed and permeabilized prokaryotic cells undergoes rRNA and gDNA degradation in a single degradation reaction volume comprising 40 million fixed and permeabilized prokaryotic cells or less and/or 2 million fixed and permeabilized prokaryotic cells/µL or less. In one example embodiment, a set of fixed and permeabilized prokaryotic cells undergoes rRNA and gDNA degradation occurs in a plurality of degradation reaction volumes each comprising 40 million fixed and permeabilized prokaryotic cells or less and/or 2 million fixed and permeabilized prokaryotic cells/µL or less.

### Labeling Each mRNA in Fixed and Permeabilized Cells

In example embodiments, after separating the set of fixed and permeabilized prokaryotic cells into a plurality of first reaction volumes, the remaining RNA in each fixed and permeabilized cell is labeled with a first barcode via reverse transcribing RNA into cDNA using primers comprising a first cell barcode. A poly-nucleotide tail (e.g., poly A tail) may then be added to the 3′ end of the cDNA. The poly-nucleotide tailing may be done concurrently with or a separate reaction following the reverse transcription step.

### Reverse Transcribing the mRNA Into cDNA

In one example embodiment, reverse transcription of each mRNA into cDNA in the fixed and permeabilized cells adds a first barcode and, optionally, a unique molecular identifier (UMI) to each first strand cDNA in a first reaction volume. In preferred embodiments, a first barcode and a unique molecular identifier (UMI) is added to each first strand cDNA in a first reaction volume. In example embodiments, the first reaction volume can be tubes (such as, microcentrifuge tubes, test tubes, and the like), or wells (such as wells in a plate). In example embodiments, the first reaction volumes are the wells of a microplate, such as a 96 well, a 384 well, or a 1536 well microplate. In an example embodiment, cells are separated into different first reaction volumes, each volume including a reverse transcription primer having the same cell barcode, but different from the barcode in any other volume. In example embodiments, there are at least 96 discrete first reaction volumes, each comprising a different barcode sequence. In example embodiments, there are at least 384 discrete first reaction volumes, each comprising a different barcode sequence. In example embodiments, the set of fixed and permeabilized prokaryotic cells are separated into a plurality of first reaction volumes at about 2 million fixed and permeabilized prokaryotic cells/first reaction volume or less. Thus, after reverse transcription, each of the about 2 million cells in each first reaction volume is labeled with the same first barcode, but the cells from each different first volume have a different first barcode. As described further herein, pooling of the cells and then splitting into second reaction volumes for adding a second cell barcode results in single cells having unique barcodes. RT primers

In one example embodiment, a first reaction volume comprises reverse transcription reagents. In one example embodiment, reverse transcription reagents comprise RT primers. In one example embodiment, RT primers comprise one or more of a 5′ primer binding sequence (PBS), a barcode sequence, and a unique molecular identifier (UMI). In one example embodiment, a barcode sequence is the same for all primers in a same first reaction volume but differs from a barcode sequence of primers in any other first reaction volume. In one example embodiment, a unique molecular identifier (UMI) is different for each primer in a first reaction volume. In one example embodiment, the RT primers include random hexamers for RT priming of all RNAs in the fixed and permeabilized cells. In an example embodiment, each RT primer comprises from 5′ to 3′, a primer binding sequence (PBS), a unique molecular identifier (UMI), a first cell barcode sequence, and a random hexamer for RT priming.

### Nucleic Acid Barcode, Barcode, and Unique Molecular Identifier (UMI)

The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. Although it is not necessary to understand the mechanism of an invention, it is believed that the barcode sequence provides a high-quality individual read of a barcode associated with a single cell, such that multiple species can be sequenced together. Barcodes can also allow for identification and/or quantification of individual sequencing-reads.

In one example embodiment barcoding uses an error correcting scheme (T. K. Moon, Error Correction Coding: Mathematical Methods and Algorithms (Wiley, New York, ed. 1, 2005)).

In preferred embodiments, unique molecular identifiers (UMI) are used. The term “unique molecular identifiers” (UMI) as used herein refers to a sequencing linker or a subtype of nucleic acid barcode used in a method that uses molecular tags to detect and quantify unique products (e.g., individual transcripts). A UMI is used to distinguish effects through a single clone from multiple clones. The term “clone” as used herein may refer to a single mRNA or target nucleic acid to be sequenced. The UMI may be used to determine the number of transcripts in each single cell, as each individual transcript will have a different UMI that can be identified even after amplification (See e.g., Islam S. et al., 2014. Nature Methods No: 11, 163-166). In one example embodiment, an UMI is a random sequence of between 4 and 20 base pairs. Not being bound by a theory, the UMI’s are designed such that assignment to the original can take place despite up to 4-7 errors during amplification or sequencing.

Unique molecular identifiers can be used, for example, to normalize samples for variable amplification efficiency. For example, in various embodiments, featuring a solid or semisolid support (for example a bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. The nucleotide barcode can be a barcode having intervening sequences. For example, a barcode for identifying a single cell may include a first and second cell barcode sequence that are non-contiguous, but together can be used to identify RNA molecules originating from the same cell.

Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

In some embodiments, a nucleic acid identifier (for example, a nucleic acid barcode) may be attached to sequences that allow for amplification and sequencing (for example, SBS3 and P5 elements for Illumina sequencing). In one example embodiment, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type NNNNNNNNNNNN.

Target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In one example embodiment, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites.

### Barcode With Cleavage Sites

In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, a cleavage site is an enzymatic cleavage site, such an endonuclease site present in a specific nucleic acid sequence. In still other embodiments, a cleavage site is a site of chemical cleavage.

### Barcode Adapters

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In one example embodiment, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/ barcode concatemer.

### Other Barcoding Embodiments

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

### Adding Poly-Nucleotide Tail Sequence

In one example embodiment, the methods comprise tailing the 3′ end of each first strand cDNA after the reverse transcribing step. In other embodiments, any tail sequence that can be added to the 3′ end of each first strand cDNA can be used and a complementary primer to the tail sequence can be used in second strand synthesis. In an example embodiment, a poly-nucleotide tail is added. In an example embodiment, a poly A, C, T, or G tail is added. In preferred embodiments, a poly-A tail is added. Non-limiting methods of tailing cDNA are known in the art, such as with terminal transferase (TdT). Terminal transferase (TdT) is a template independent polymerase that catalyzes the addition of deoxynucleotides to the 3′ hydroxyl terminus of DNA molecules. Protruding, recessed or blunt-ended double or single-stranded DNA molecules serve as a substrate for TdT. The 58.3 kDa enzyme does not have 5′ or 3′ exonuclease activity. The addition of Co2+ in the reaction makes tailing more efficient. The rate of addition of dNTP’s and thus the length of the tail is a function of the ratio of 3′ DNA ends: dNTP concentration, and also which dNTP is used. In example embodiments, TdT is used to add a poly-A tail. In example embodiments, TdT is used to add a poly-C, -T, or -G tail. In certain embodiments, more than one nucleotide is used to tail the 3′ end, such as by tailing with a first nucleotide, washing out the first nucleotide and a performing an additional TdT reaction with a second nucleotide different from the first.

In one example embodiment, the fixed and permeabilized cells from the plurality of first reaction volumes is pooled into a single tailing reaction volume before adding a tail sequence. In one example embodiment, pooling fixed and permeabilized cells comprises only pooling fixed and permeabilized cells from the same permeabilizing reaction volume into a single tailing reaction volume. In one example embodiment, pooling fixed and permeabilized cells before tailing is optional. In one example embodiment, a set of fixed and permeabilized prokaryotic cells undergoes tailing in a tailing reaction volume comprising 40 million fixed and permeabilized prokaryotic cells and/or 650,000-800,000 fixed and permeabilized prokaryotic cells/µL.

### Labeling Each cDNA From the Same Cell With a Unique Barcode

Individual fixed and permeabilized cells comprising the labeled first strand cDNA are then separated into individual discrete volumes where second strand synthesis of cDNA and second round barcoding is conducted in the fixed and permeabilized cells. The second strand cDNA synthesis and second round barcoding results in addition of a second barcode and, optionally, a unique molecular identifier (UMI). For example, the primer sequence including the second barcode can also include a UMI. The second barcode is unique to each individual discrete volume such that all cDNA from the same individual discrete volume receive the same barcode sequence and thus the second barcode in combination with the first barcode can be distinguished as arising from the same individual cell. This is based on the low probability that two cells from the same first reaction volume will be in the same individual discrete second reaction volume.

### Individual Discrete Volumes

An “individual discrete volume” is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents maybe passed in through the discrete volume, while other material, such as target molecules, maybe maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate.

In one example embodiment, at least one fixed and permeabilized cell is separated into each second reaction volume. In one example embodiment, at least two or more fixed and permeabilized cells are separated into each second reaction volume. In one example embodiment, at least one, at least two, at least three, at least four, at least five, or at least six fixed and permeabilized cells are separated into each second reaction volume. In one example embodiment, one, two, three, four, five, six, seven, eight, nine, or ten fixed and permeabilized cells are separated into each second reaction volume. In preferred embodiments, about six fixed and permeabilized cells are separated into each second reaction volume (e.g., 5, 6, 7 cells). In one example embodiment, there is a Poisson distribution of numbers of cells in each second reaction volume, but the parameter λ, (the expected rate of occurrences), which is usually 0.1-0.3 in traditional drop-seq and 10x single-cell sequencing, is increased to >1 to 10, or even higher depending on the number of first cell barcodes in the first reaction volume (e.g., 384 first cell barcodes). In probability theory and statistics, the Poisson distribution is a discrete probability distribution that expresses the probability of a given number of events occurring (index k) in a fixed interval of time or space if these events occur with a known constant mean rate λ, (the expected rate of occurrences) and independently of the time since the last event. The average cell number in each second reaction volume is the same as λ. In one example embodiment, the number of cells in the second reaction volume is not limited to 6. In one example embodiment, there are 3-6 cells in the second reaction volume, which have a low barcode collision rate when there are 384 first cell barcodes. Barcode collision rate refers to the rate that two single cells acquire the same first and second cell barcodes. In one example embodiment, there are more than 384 first cell barcodes and there are more than 6 cells on average per second reaction volume. In one example embodiment, each second reaction volume does not comprise a Poisson distribution, such that each second reaction volume receives the same number of cells.

In one example embodiment, second reaction volumes are droplets. In one example embodiment, second reaction volumes are aqueous in oil droplets. In one example embodiment, second reaction volumes are microwells.

In example embodiments, each second reaction volume also includes approximately one or less barcoded bead that is conjugated to a plurality of capture oligonucleotides comprising the second barcode sequence, such that each bead includes a second barcode sequence. The loading of 1 or less bead ensures that single cells are not double barcoded or that each single cell receives the same barcode. The second barcode sequence is the same for all capture oligonucleotides on the same bead, but differ from capture oligonucleotides on any other bead. In other words, all capture oligonucleotides on the bead will share the same barcode, which is unique to that individual bead and distinct from the barcode on any other bead. In one example embodiment, each capture oligonucleotide comprises from 5′ to 3′ a second cell barcode sequence and a capture sequence capable of hybridizing to second strand cDNA and priming linear amplification. In one example embodiment, each bead comprises a plurality of capture oligonucleotides attached 5′ to the bead surface. In one example embodiment, each capture oligonucleotide comprises a barcode sequence that is the same for all capture oligonucleotides on a same bead but differs from the barcode sequence of capture oligonucleotides on other beads. In one example embodiment, each capture oligonucleotide comprises a capture sequence that is the same as a 5′ primer binding sequence in each RT primer. Thus, in one example embodiment, beads are delivered to individual discrete second reaction volumes with fixed and permeabilized prokaryotic cells comprising first strand cDNA (e.g., 1-6 cells). In one example embodiment, the capture oligonucleotides comprise a UMI sequence.

In one example embodiment, the beads are 10X Gel Beads from a Chromium Next GEM Single Cell ATAC Library & Gel Bead Kit (10x Genomics) and the capture oligonucleotides are the (i) partial Illumina Read 1(R1) sequence (22 bp) and (ii) the 16 bp 10x Barcode on the surface of each 10X Gel Bead. The 10x Barcode sequence is unique to each Gel Bead.

The formation of droplets with single beads is used for known methods of single cell sequencing of mammalian cells. Similar or the same microfluidic devices can be used for segregating fixed and permeabilized microbial cells into droplets or microwells. In example embodiments, flow rates and bead concentrations can use standard flow rates and concentrations such that each droplet receives about 1 or no bead. The flow rate for the channel introducing the microbial cells can be the same or adjusted such that each droplet receives about 1 to 6 microbial cells. In an example embodiment, the concentration of microbial cells introduced to the microbial system is used to control for the number of cells in each droplet. Exemplary microfluidic systems for single cell RNA sequencing applicable to the present invention are described and include Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan;12(1):44-73; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 Oct;14(10):955-958; International patent application number PCT/US2016/059239, published as WO2017164936 on Sep. 28, 2017; International patent application number PCT/US2018/060860, published as WO/2019/094984 on May 16, 2019; International patent application number PCT/US2019/055894, published as WO/2020/077236 on Apr. 16, 2020; and Drokhlyansky, et al., “The enteric nervous system of the human and mouse colon at a single-cell resolution,” bioRxiv 746743.

In an exemplary embodiment, single beads are distributed to microwells and the microbial cells are separated into the microwells (see, e.g., Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273). In exemplary embodiments, the microwell can only accommodate a single bead and the dilution of the microbial cells can be used to obtain the desired number of microbial cells per microwell. The microwells may accommodate more than one microbial cell (e.g., about 6).

### Conducting Second Strand cDNA Synthesis and Second Barcoding

Second strand cDNA synthesis is accomplished in each discrete second reaction volume by amplification. Non-limiting examples of amplification are known in the art. In one example embodiment, second strand cDNA synthesis is accomplished by linear amplification. Linear amplification generally refers to a method that involve the formation of one or more copies of the complement of only one strand of a nucleic acid or polynucleotide molecule, usually a nucleic acid or polynucleotide analyte. Thus, the primary difference between linear amplification and exponential amplification is that in the latter process, the product serves as substrate for the formation of more product, whereas in the former process the starting sequence is the substrate for the formation of product but the product of the reaction, i.e., the replication of the starting template, is not a substrate for generation of products. In linear amplification the amount of product formed increases as a linear function of time as opposed to exponential amplification where the amount of product formed is an exponential function of time. The methods of the invention generally incorporate single primer isothermal amplification (SPIA). SPIA is an isothermal amplification method described, for example, in U.S. Pat. Nos. 6,692,918, 6,251,639, 6,946,251 and 7,354,717. The primer used in the second strand cDNA synthesis is selected based on its ability to hybridize to the tail sequence added during first strand cDNA synthesis above. Thus, if a specific primer binding the tail sequence was added to the first cDNA strand, the primer selected for second strand cDNA synthesis will be able to hybridize to and prime the second strand synthesis. If a poly-A tail was added during the first strand cDNA synthesis, then a poly-T primer may be used to prime the second strand synthesis. The first strand cDNA includes a primer binding site (PBS) provided by the RT primer above. Second strand synthesis generates a second strand DNA that includes the reverse complement of the PBS. The PBS of the second strand cDNA is now available for hybridization with a sequence including the PBS.

In one example embodiment, the beads provided to each discrete second reaction volume comprise a capture oligonucleotide sequence comprising the primer binding sequence (PBS), thus allowing hybridization to the second strand cDNA for priming linear amplification. In one example embodiment, the second strand cDNA is captured by the capture oligonucleotides on the bead in the second reaction volume and the cDNA is amplified by linear amplification to generate cDNA that includes a first and second cell barcode and a UMI sequence.

In another exemplary embodiment, the beads can include capture oligonucleotides with the reverse complement of the PBS as the capture sequence. In this embodiment, first strand cDNA is directly captured by the capture oligonucleotide and extended by linear amplification to generate barcoded second strand cDNA. This second strand cDNA can be further amplified to generate the cDNA for sequencing.

### Preparing a Sequencing Library

In one example embodiment, methods further comprise preparing a sequencing library. In certain aspects, a sequencing library comprises amplified labeled cDNA. In one example embodiment, a single cell cDNA library is prepared. Methods for constructing sequencing libraries are known in the art (see, e.g., Head et al., Library construction for next-generation sequencing: Overviews and challenges. Biotechniques. 2014; 56(2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi:10.1002/0471142727.mb0422s107 (2014). PMCID:4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina’s reversible terminator method, long read nanopore sequencing, Roche’s pyrosequencing method (454), Life Technologies’ sequencing by ligation (the SOLiD platform) or Life Technologies’ Ion Torrent platform. Examples of such methods are described in the following references: Margulies et al (Nature 2005 437: 376-80); Schneider and Dekker (Nat Biotechnol. 2012 Apr 10;30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In one example embodiment, a sequencing library is able to distinguish species heterogeneity, strain heterogeneity, genotypic heterogeneity and/or phenotypic heterogeneity of the plurality of prokaryotic cells. In one example embodiment, a sequencing library is able to distinguish species heterogeneity, strain heterogeneity, genotypic heterogeneity and/or phenotypic heterogeneity of one or more uncharacterized organisms, an environmental microbiome, or an organismal microbiome. In one example embodiment, a sequencing library can distinguish phenotypic heterogeneity in the response of the plurality of prokaryotic cells to one or more drug treatments. In one example embodiment, a sequencing library can distinguish phenotypic heterogeneity in the response of the plurality of prokaryotic cells to one or more antibiotic treatments.

### Perturbation and Library Screening

The methods of single prokaryotic cell analysis can be utilized for evaluating environmental stress and/or state, for screening of chemical libraries, and to screen or identify structural, syntenic, genomic, and/or organism and species variations. Aspects of the present disclosure relate to the correlation of an environmental stress or state with the gene expression profile in a sample of cells, for example a culture of cells, can be exposed to an environmental stress, such as but not limited to heat shock, osmolarity, hypoxia, cold, oxidative stress, radiation, starvation, a chemical (for example a therapeutic agent or potential therapeutic agent, such as an antibiotic) and the like. After the stress is applied, a representative sample can be subjected to analysis, for example at various time points, and compared to a control, such as a sample from an organism or cell, for example a cell from an organism, or a standard value.

In some embodiments, the disclosed methods can be used to screen chemical libraries for agents that modulate gene expression profiles, and/or relationships thereof. By exposing prokaryotic cells to different members of the chemical libraries, and performing the methods described herein, different members of a chemical library can be screened for their effect on gene expression profiles, and/or relationships thereof simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

### Screening for Modulating Agents

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells (e.g., antibiotic resistance). In one example embodiment, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively – for example, where modulation comprises or consists of a change in a quantifiable variable such as a quantifiable property of a cell or where a quantifiable variable provides a suitable surrogate for the modulation – modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In one example embodiment, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. Prokaryotic cell gene signatures or biological programs may be identified using the present invention. Prokaryotic cell gene signatures or biological programs may be used to screen for drugs that reduce the signature or biological program in cells as described herein. The signature or biological program may be used for GE-HTS. In one example embodiment, pharmacological screens may be used to identify drugs that are selectively toxic to cells having a signature.

### Perturb-seq

In one example embodiment, the gene signatures and gene networks are identified by perturbation of target genes within said prokaryotic cells. Methods and tools for genome-scale screening of perturbations in single cells using CRISPR-Cas9 have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867–1882; Feldman et al., Lentiviral co-packaging mitigates the effects of intermolecular recombination and multiple integrations in pooled genetic screens, bioRxiv 262121, doi: doi.org/10.1101/262121; Datlinger, et al., 2017, Pooled CRISPR screening with single-cell transcriptome readout. Nature Methods. Vol.14 No.3 DOI: 10.1038/nmeth.4177; Hill et al., On the design of CRISPR-based single cell molecular screens, Nat Methods. 2018 Apr; 15(4): 271-274; Replogle, et al., “Combinatorial single-cell CRISPR screens by direct guide RNA capture and targeted sequencing” Nat Biotechnol (2020). doi.org/10.1038/s41587-020-0470-y; Schraivogel D, Gschwind AR, Milbank JH, et al. “Targeted Perturb-seq enables genome-scale genetic screens in single cells”. Nat Methods. 2020;17(6):629-635; Frangieh CJ, Melms JC, Thakore PI, et al. Multimodal pooled Perturb-CITE-seq screens in patient models define mechanisms of cancer immune evasion. Nat Genet. 2021;53(3):332-341; and International publication serial number WO/2017/075294). The present invention is compatible with perturb-seq, such that prokaryotic genes may be perturbed and the perturbation may be identified and assigned to the gene expression readouts of single cells. In one example embodiment, the perturbation and barcode sequence is expressed from a bacterial promoter and does not require polyadenylated RNAs. In one example embodiment, signature genes may be perturbed in single cells and gene expression analyzed. Not being bound by a theory, networks of genes that are disrupted due to perturbation of a gene may be determined. Understanding the network of genes effected by a perturbation may allow for a gene to be linked to a specific pathway that may be targeted to modulate the signature and treat a disease. Thus, in one example embodiment, perturb-seq is used to discover novel drug targets to allow treatment of specific bacterial infections.

The perturbation methods and tools allow reconstructing of a cellular network or circuit. In one embodiment, the method comprises (1) introducing single-order or combinatorial perturbations to a population of cells, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In one example embodiment, the measuring of phenotypic differences and assigning a perturbation to a single cell is determined by performing single prokaryotic cell RNA sequencing as described herein. In one example embodiment, a guide RNA is detected by RNA-seq using a transcript expressed from a vector encoding the guide RNA. The transcript may include a unique barcode specific to the guide RNA. Not being bound by a theory, a guide RNA and guide RNA barcode is expressed from the same vector and the barcode may be detected by RNA-seq. Not being bound by a theory, detection of a guide RNA barcode is more reliable than detecting a guide RNA sequence, reduces the chance of false guide RNA assignment and reduces the sequencing cost associated with executing these screens. Thus, a perturbation may be assigned to a single cell by detection of a guide RNA barcode in the cell. In one example embodiment, a cell barcode is added to the RNA in single cells, such that the RNA may be assigned to a single cell. Generating cell barcodes is described herein for single cell sequencing methods. In one example embodiment, a Unique Molecular Identifier (UMI) is added to each individual transcript. Not being bound by a theory, the UMI allows for determining the capture rate of measured signals or the number of transcripts captured.

In one example embodiment, other CRISPR-based perturbations are readily compatible with Perturb-seq, including alternative editors such as CRISPR/Cpf1. In one example embodiment, Perturb-seq uses Cpf1 as the CRISPR enzyme for introducing perturbations. Not being bound by a theory, Cpf1 does not require Tracr RNA and is a smaller enzyme, thus allowing higher combinatorial perturbations to be tested.

In one embodiment, perturbation is by deletion of regulatory elements. Non-coding elements may be targeted by using pairs of guide RNAs to delete regions of a defined size, and by tiling deletions covering sets of regions in pools.

In one example embodiment, whole genome screens can be used for understanding the phenotypic readout of perturbing potential target genes. In preferred embodiments, perturbations target expressed genes as defined by a gene signature using a focused sgRNA library. Libraries may be focused on expressed genes in specific networks or pathways. In other preferred embodiments, regulatory drivers are perturbed. Applicants can use gene expression profiling data to define the target of interest and perform follow-up single-cell and population RNA-seq analysis.

The invention is further described by the following numbered paragraphs:
1. A method of generating a single microbial cell cDNA library, wherein the RNAs from different microbial cells are barcoded individually, allowing the cell identity of each RNA to be retained in a single library, the method comprising:
   a. fixing each cell of a plurality of microbial cells within one or more first reaction volumes;
   b. permeabilizing each fixed cell within one or more second reaction volumes;
   c. degrading ribosomal RNA (rRNA) in each fixed and permeabilized cell, wherein at least a portion of the rRNA in each cell is degraded;
   d. degrading DNA in each fixed and permeabilized cell, wherein at least a portion of the DNA in each cell is degraded;
   e. segregating at least a portion of or all of the fixed and permeabilized cells into separate third reaction volumes, each third reaction volume comprising RT primers, wherein each third reaction volume comprises one or more of the fixed and permeabilized cells, and wherein each RT primer comprises (i) a 5′ primer binding sequence (PBS) (ii) a first barcode sequence that is the same for all primers in the same third reaction volume but differs from the barcode sequence of primers in any other third reaction volume, and (iii) a unique molecular identifier (UMI) sequence that is different for each primer in a third reaction volume;
   f. reverse transcribing the remaining RNA in each fixed and permeabilized cell using the RT primers to produce labeled individual first strand cDNA;
   g. adding a common priming sequence to the 3′ end of the first strand cDNA in each fixed and permeabilized cell;
   h. merging one or more of the fixed and permeabilized cells and a single bead into separate fourth reaction volumes comprising a plurality of primers capable of hybridizing to the common priming sequence added in step (g), wherein each bead comprises a plurality of capture oligonucleotides attached 5′ to the bead surface, each capture oligonucleotide comprising (i) a second barcode sequence that is the same for all capture oligonucleotides on the same bead but differs from the barcode sequence of capture oligonucleotides on other beads and (ii) a capture sequence that is the same as the 5′ primer binding sequence (PBS) in each RT primer;
   i. hybridizing the primers added at step (h) to the common priming sequence added at the 3′ end of the 1st strand cDNA in the fixed and permeabilized cells in the separate fourth reaction volumes;
   j. generating 2nd strand cDNA by linear amplification using the primers hybridized to the common priming sequence added at the 3′ end of the 1st strand cDNA;
   k. generating cDNA comprising the bead barcode sequence by amplifying the 2nd strand cDNA using the capture sequence on the capture oligonucleotides attached to the bead to prime linear amplification; and
   1. pooling the cDNA from the separate fourth reaction volumes, whereby a library of cDNA comprising a second barcode, UMI and first barcode is obtained.
2. The method of paragraph 1, wherein 10 billion microbial cells or less are fixed in each of the first reaction volumes.
3. The method of paragraph 1 or 2, wherein 40 million fixed cells or less are permeabilized in each of the second reaction volumes.
4. The method of any of paragraphs 1 to 3, wherein about 1,000 to 1 million fixed and permeabilized cells are segregated into the separate third reaction volumes.
5. The method of paragraph 4, wherein at least 10,000 fixed and permeabilized cells are segregated into the separate third reaction volumes.
6. The method of paragraph 4, wherein at least 30,000 fixed and permeabilized cells are segregated into the separate third reaction volumes comprise.
7. The method of paragraph 4, wherein at least 140,000 fixed and permeabilized cells are segregated into the separate third reaction volumes.
8. The method of any of paragraphs 1 to 7, wherein mRNA accounts for more than 5%, more than 10%, more than 20%, more than 30%, more than 40%, more than 50%, more than 60%, more than 70%, or more than 80% of total aligned reads.
9. The method of any of paragraphs 1 to 8, wherein the mean mRNA UMI per cell is more than 25, more than 35, more than 45, more than 55, or more than 65.
10. The method of any of paragraphs 1 to 9, wherein the fixing step is accomplished by addition of formaldehyde or paraformaldehyde.
11. The method of any of paragraphs 1 to 10, wherein the permeabilizing step comprises treatment with ethanol, detergent and/or lysozyme.
12. The method of any of paragraphs 1 to 11, wherein the degrading rRNA step is accomplished by addition of rRNA selective DNA probes and RNase H.
13. The method of any of paragraphs 1 to 12, wherein the degrading DNA step is accomplished by addition of DNase I.
14. The method of any of paragraphs 1 to 13, wherein the common priming sequence is a poly A sequence, such as SMRT_dT.
15. The method of paragraph 14, wherein the polyA sequence is added by a Terminal Transferase (TdT).
16. The method of any of paragraphs 1 to 15, wherein the method further comprises a lysing step accomplished by heating at about 98° C. for 2 minutes in the fourth reaction volume.
17. The method of any of paragraphs 1 to 16, wherein each third reaction volume is a microwell.
18. The method of any of paragraphs 1 to 17, wherein each fourth reaction volume can be loaded with multiple fixed and permeabilized cells.
19. The method of any of paragraphs 1 to 18, wherein each fourth reaction volume comprises one, two, three, four, five, six, or seven fixed and permeabilized cells.
20. The method of any of paragraphs 1 to 19, wherein each fourth reaction volume is an aqueous in oil droplet.
21. The method of any of paragraphs 1 to 20, wherein the bead material is a hydrogel, polystyrene, methylstyrene, acrylic polymer, paramagnetic material, ceramic, plastic, glass, titanium, latex, sepharose, cellulose, or nylon.
22. The method of paragraph 21, wherein the bead material is methacrylate resin.
23. The method of any of paragraphs 1 to 20, wherein the droplets are formed by a microfluidic device configured for merging microbial cells and beads into aqueous in oil droplets.
24. The method of any of paragraphs 1 to 23, wherein the plurality of microbial cells comprises cells from one or more bacterial species selected from the group consisting of an Acinetobacter species, an Actinobacillus species, an Actinomycetes species, an Actinomyces species, Aerococcus species an Aeromonas species, an Anaplasma species, an Alcaligenes species, a Bacillus species, a Bacteroides species, a Bartonella species, a Bifidobacterium species, a Bordetella species, a Borrelia species, a Brucella species, a Burkholderia species, a Campylobacter species, a Capnocytophaga species, a Chlamydia species, a Citrobacter species, a Coxiella species, a Corynbacterium species, a Clostridium species, an Eikenella species, an Enterobacter species, an Escherichia species, an Enterococcus species, an Ehlichia species, an Epidermophyton species, an Erysipelothrix species, a Eubacterium species, a Francisella species, a Fusobacterium species, a Gardnerella species, a Gemella species, a Haemophilus species, a Helicobacter species, a Kingella species, a Klebsiella species, a Lactobacillus species, a Lactococcus species, a Listeria species, a Leptospira species, a Legionella species, a Leptospira species, Leuconostoc species, a Mannheimia species, a Microsporum species, a Micrococcus species, a Moraxella species, a Morganell species, a Mobiluncus species, a Micrococcus species, Mycobacterium species, a Mycoplasm species, a Nocardia species, a Neisseria species, a Pasteurelaa species, a Pediococcus species, a Peptostreptococcus species, a Pityrosporum species, a Plesiomonas species, a Prevotella species, a Porphyromonas species, a Proteus species, a Providencia species, a Pseudomonas species, a Propionibacteriums species, a Rhodococcus species, a Rickettsia species, a Rhodococcus species, a Serratia species, a Stenotrophomonas species, a Salmonella species, a Serratia species, a Shigella species, a Staphylococcus species, a Streptococcus species, a Spirillum species, a Streptobacillus species, a Treponema species, a Tropheryma species, a Trichophyton species, an Ureaplasma species, a Veillonella species, a Vibrio species, a Yersinia species, and a Xanthomonas species.
25. The method of any of paragraphs 1 to 24, wherein the plurality of microbial cells comprise cells from one or more bacterial species selected from the group consisting of Escherichia coli, Klebsiella pneumoniae, Pseudomonas aeruginosa, and Enterococcus faecium.
26. The method of any of paragraphs 1 to 25, wherein the plurality of microbial cells comprise one or more uncharacterized organisms, an environmental microbiome, or an organismal microbiome.
27. The method of any of paragraphs 1 to 26, wherein the method can distinguish species heterogeneity, strain heterogeneity, genotypic heterogeneity and/or phenotypic heterogeneity of the plurality of microbial cells.
28. The method of any of paragraphs 1 to 27, wherein the method can distinguish phenotypic heterogeneity in the response of the plurality of microbial cells to one or more drug treatments.
29. The method of any of paragraphs 1 to 28, wherein the method can distinguish phenotypic heterogeneity in the response of the plurality of microbial cells to one or more antibiotic treatments.
30. The method of any of paragraphs 1 to 29, wherein the plurality of microbial cells is a biological sample obtained from a subject.

Further embodiments are illustrated in the following Examples which are given for illustrative purposes only and are not intended to limit the scope of the invention.

### EXAMPLES

### Example 1 -BacDrop: Droplet-Based, High Throughput Technology for Bacterial Single-Cell RNA Sequencing (scRNA-Seq)

Heterogeneity plays critical roles in bacterial survival and evolution. While single-cell RNA sequencing (scRNA-seq) has revolutionized the study of cellular heterogeneity in eukaryotic biology and disease, its application to bacteria is still limited due to technical and biological challenges. Here Applicants introduce BacDrop, a bacterial droplet-based high throughput scRNA-seq technology that has overcome many of these challenges. BacDrop has the flexibility to be applied to a range of cell numbers, from thousands to millions, and across both gram-negative and gram- positive bacterial species. It features universal ribosomal RNA depletion and combinatorial barcodes that enable multiplexing. Applicants applied BacDrop to study Klebsiella pneumoniae clinical isolates and to elucidate their critical, heterogenous responses to antibiotic stress. In an unperturbed population previously presumed to be homogenous, Applicants find within population heterogeneity, with subpopulations defined on a genome-wide level, driven largely by expression of mobile genetic elements. In the dynamic setting after antibiotic perturbation, BacDrop revealed transcriptionally distinct subpopulations previously masked by bulk analysis, that are associated with different phenotypic outcomes including decreased antibiotic efficacy and persister formation. BacDrop thus enables the detection of within population heterogeneity and critical transcriptional states that cannot be detected using bulk RNA-seq, especially when performed on large numbers of cells. Importantly, BacDrop can enable new microbiological insights, including characterizations of subpopulations within a larger community such as the microbiome or understanding of heterogenous responses to perturbation such as environmental stress, encounters with a host, residence in a biofilm, or antibiotic exposure.

Here Applicants report the development and application of BacDrop, a droplet-based high throughput bacterial scRNA-seq technology. BacDrop can investigate millions of single bacterial cells simultaneously, without requiring prior knowledge of the genome or probe-design. Due to the paucity of mRNA content in a single bacterial cell and the extremely low fraction of cells in a population that account for phenomena such as persistence or heteroresistance^{1,6,12,13}, a larger number of cells are needed for single-cell analysis to confidently assess differential expression and to identify rare populations, depending on the prevalence of the phenomenon of interest. In addition, if applied to samples with high complexity and diversity, such as microbiota samples that usually contains hundreds of species, large numbers of cells are critical to ensure the capture of enough cells from each species. BacDrop also includes a universal and efficient rRNA-depletion step, reducing sequencing costs by at least ten-fold, thus enabling higher throughput studies. Furthermore, Applicants demonstrated that BacDrop works on a variety of bacterial species, including the gram-negative Escherichia coli, Klebsiella pneumoniae, Pseudomonas aeruginosa, and the gram-positive bacterium Enterococcus faecium.

Applicants applied BacDrop to determine any within population heterogeneity of K. pneumoniae and characterize its responses to antibiotics. K.pneumoniae is a gram-negative pathogen which has become one of the leading threats in the antibiotic resistance crisis, with increasing numbers of strains resistant to carbapenems, the last-resort antibiotic used to treat the most resistant infections¹⁴. Under stable and dynamic conditions, with and without antibiotic perturbations, Applicants identified important new features of heterogeneity that has heretofore been masked by bulk analysis in K.pneumoniae clinical isolates. In the absence of antibiotic perturbation, Applicants found within population heterogeneity driven predominantly by mobile genetic elements (MGEs), thus demonstrating novel subpopulation structure in a previously presumed homogenous population. Applicants then measured the transcriptional response of a K. pneumoniae clinical isolate after perturbation with antibiotics and found heterogenous responses on the single-cell level. After antibiotic perturbation, BacDrop revealed transcriptionally distinct subpopulations previously masked by bulk analysis that are associated with different phenotypic outcomes including decreased antibiotic efficacy and persister formation. Specifically, a diverse range of stress responses was induced in different subpopulations rather than inducing a single or uniform response in all cells after specific antibiotic exposure. For example, after treatment by the carbapenem antibiotic meropenem, cspD, a DNA/RNA binding toxin that inhibits DNA replication and induces persister formation^{15,16}, was highly expressed in a subpopulation of K.pneumoniae. Importantly, meropenem had reduced efficacy in this subpopulation, which was enriched for persisters, thereby demonstrating the potential functional importance of these subpopulations. With this demonstration of the power of BacDrop to illuminate heterogeneity both in static bacterial populations and their dynamic responses to perturbations, Applicants propose that BacDrop has the potential to transform Applicants’ understanding of bacterial survival, adaptation, and evolution, with numerous potential applications including the characterization of complex communities.

### Results

### BacDrop: A Bacterial Droplet-based High Throughput scRNA-seq Technology

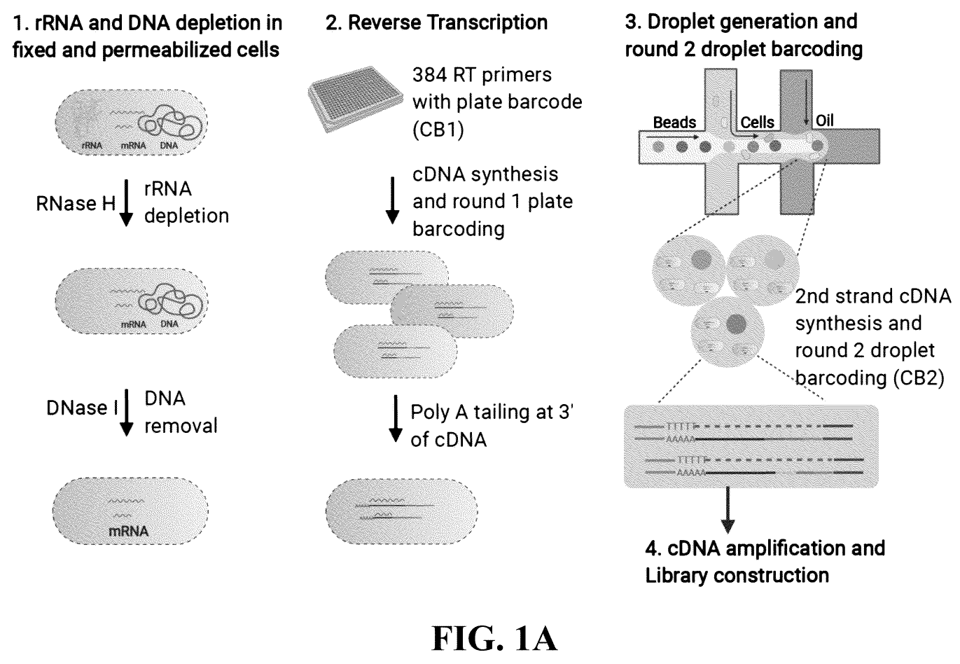

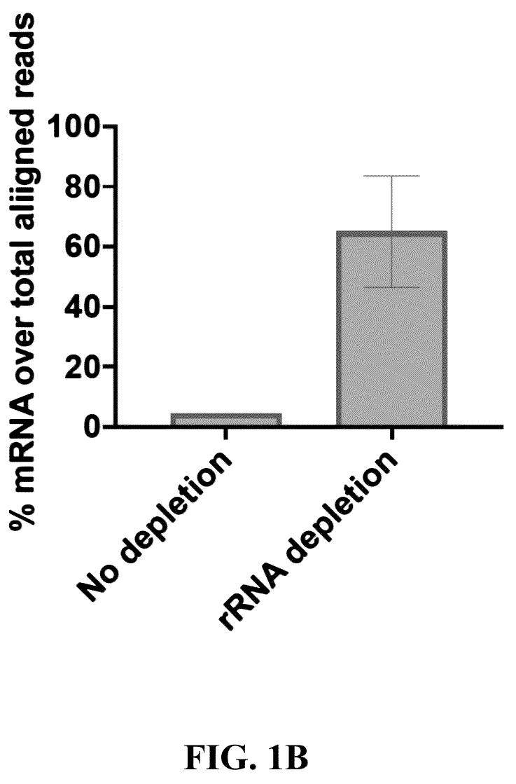

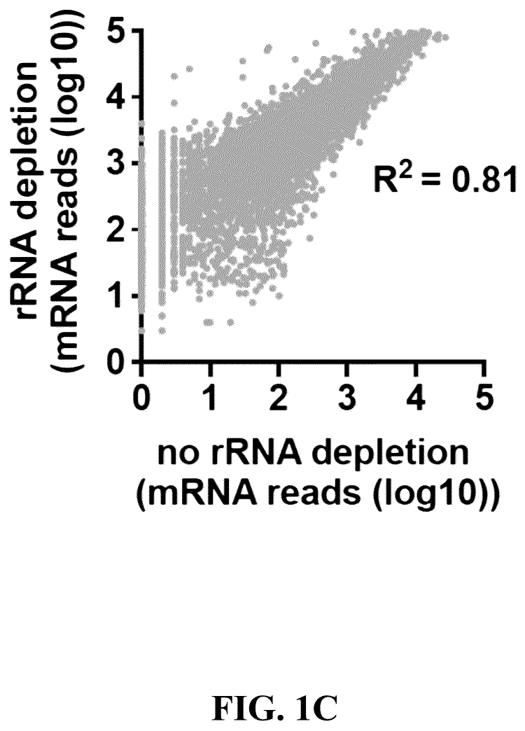

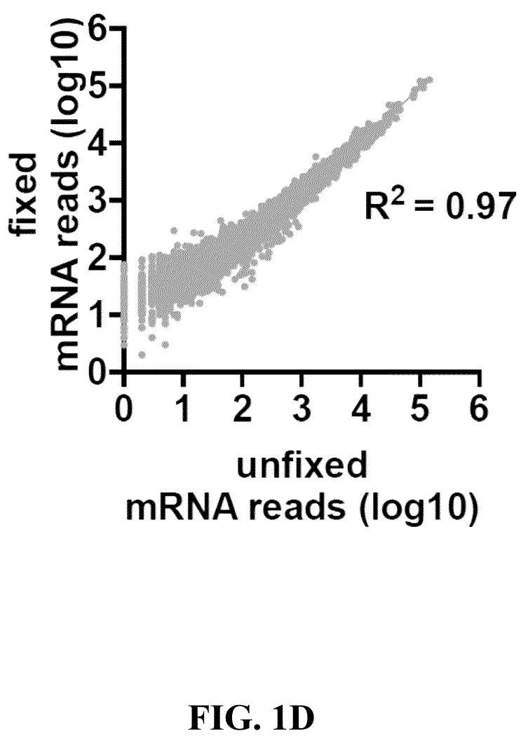

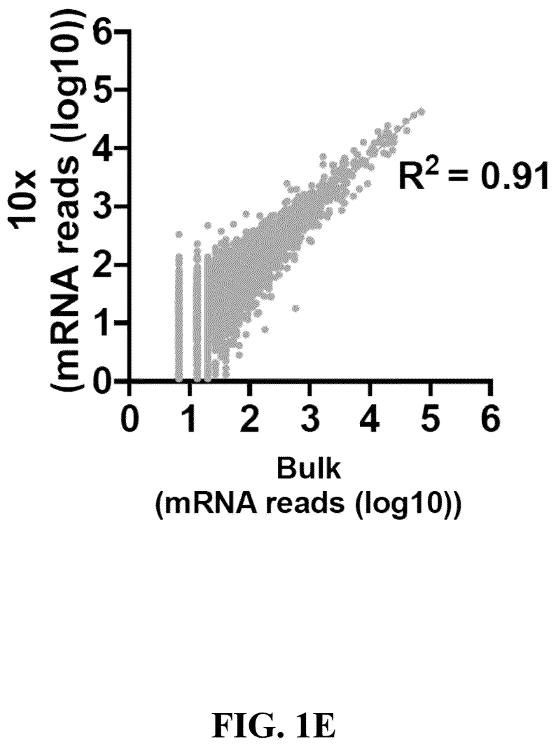

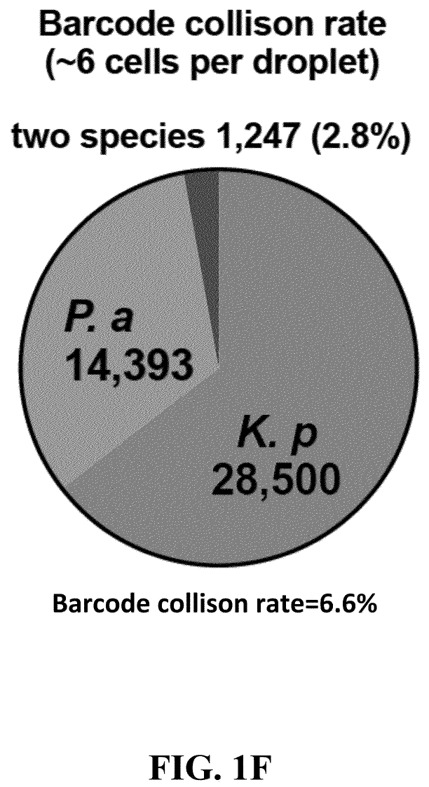

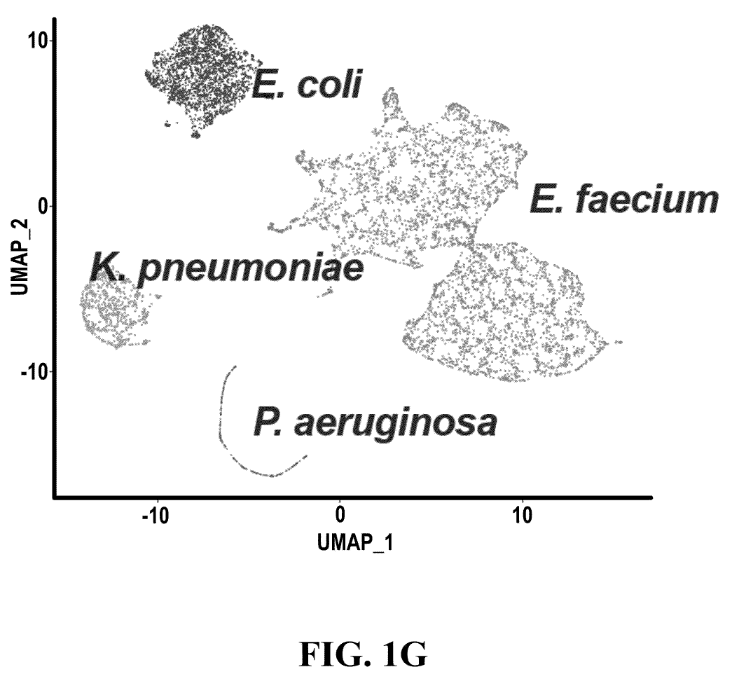

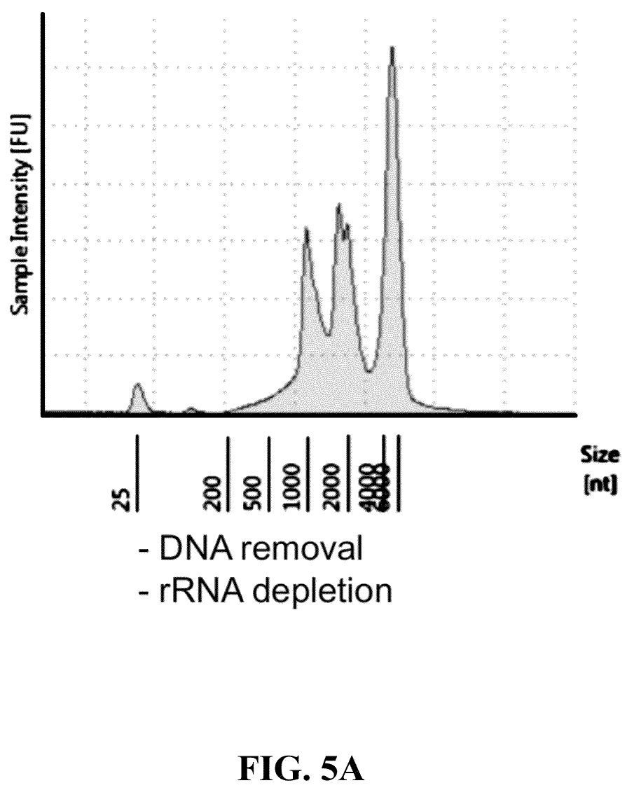

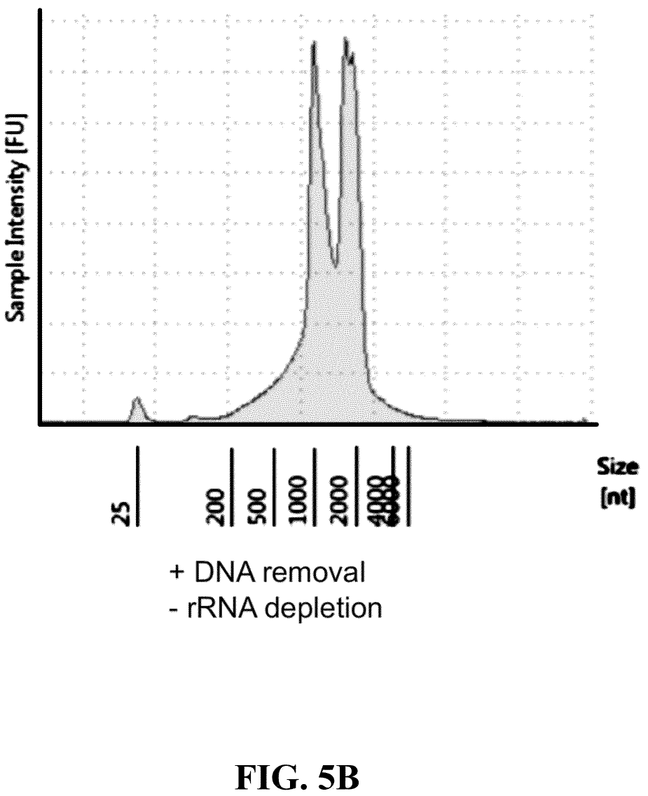

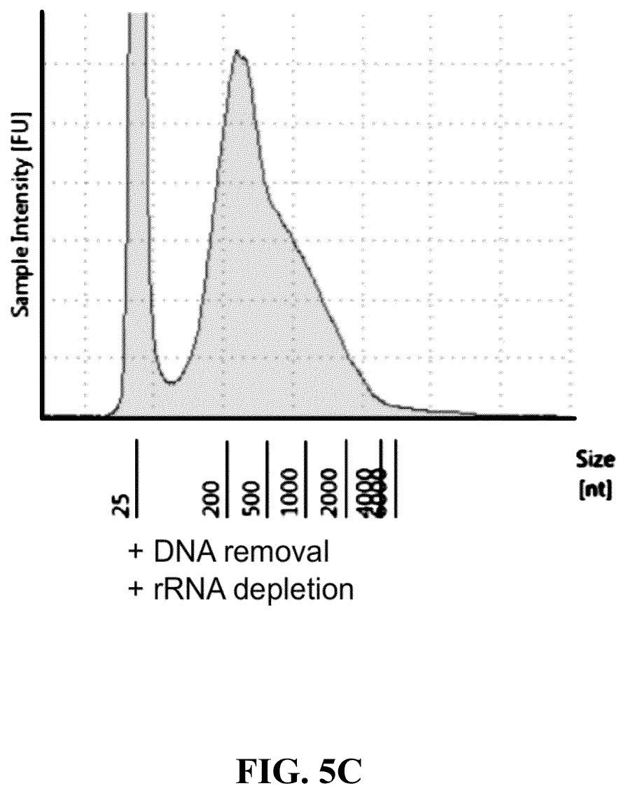

Applicants developed BacDrop based on droplet technology, which has the advantage of achieving much higher throughput than plate-based technologies, leveraging the 10x Genomics™ platform, a reliable and commercially available droplet-based platform for scRNA-seq. Two of the unique features of bacteria compared to mammalian cells are the challenge of lysing the bacterial cell wall, which requires harsher lysis conditions, while preserving RNA integrity, and the absence of mRNA polyadenylated tails, thus requiring an alternative approach to isolate mRNA (~5%) from the vastly more abundant rRNA (~95%). To overcome these problems, Applicants adapted a previously reported cell fixation and permeabilization protocol prior to droplet encapsulation to avoid cell lysis^{7,9}, and implemented universal rRNA and genomic DNA (gDNA) depletion steps within the permeabilized, fixed cell using RNase H and DNase I, respectively (FIG. 1A, FIGS. 5A-C).

Droplet-based single cell approaches require Poisson loading at mean droplet occupancy values (λ) far less than 1 to ensure that multiple cells are not encapsulated within a single droplet¹⁷ and thus do not share the same barcode. Because of this low occupancy value, Poisson loading limits numbers of cells that can be loaded in each microfluidic channel. To avoid this problem, Applicants utilized a multi-step barcoding strategy wherein cells are uniquely identified using the combination of two barcodes: a “plate barcode” (CB 1) as a pre-indexing step corresponding to one of a possible 384 different barcodes, and a second “droplet barcode” (CB2) that uniquely identifies each droplet, together constituting the cell barcode. A unique molecular identifier (UMI) is also included as part of CB1 to enable identification of unique transcripts via removal of PCR duplicates. This pre-indexing strategy has recently been successfully applied to mammalian droplet-based scRNA-seq¹⁸.

The pre-indexing with CB 1 was achieved via reverse transcription (RT) reactions using random hexamer priming in a 384-well plate (plate barcoding), with ~200,000 cells in each well containing one of 384 uniquely barcoded RT primers¹⁸ (FIG. 1A, Table 1). Template switching is less efficient for second strand cDNA synthesis from bacterial than mammalian mRNA due to the lack of proper modifications at the 5′ end of bacterial mRNA. Applicants thus used Terminal Transferase (TdT) to append poly-A tails to the 3′ end of cDNA to facilitate second strand cDNA synthesis inside droplets. By pre-indexing,Applicants were able to load each droplet with multiple cells, making Poisson loading unnecessary and increasing the throughput significantly from a λ, of ~0.3 for conventional droplet scRNA-seq experiments to a λ, of >1 for Applicants’ approach with multiple cells in each drop.

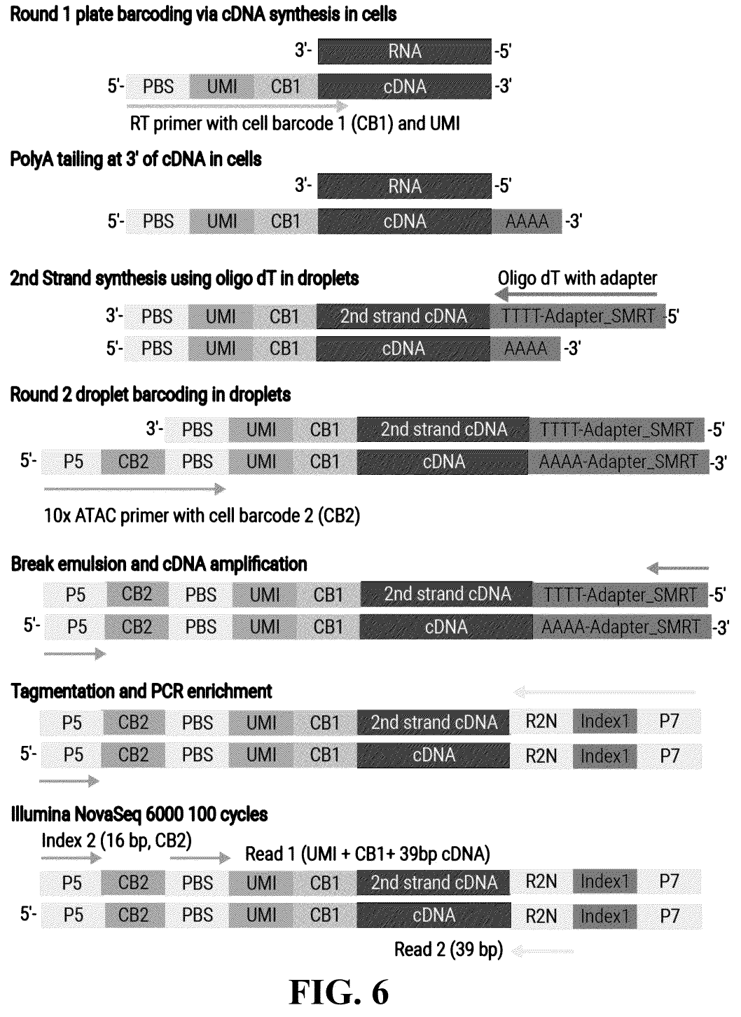

For droplet generation and round 2 droplet barcoding (CB2), Applicants utilized the commercially available single-cell kit from 10x Genomics™ (see methods) and loaded 3-6 cells per droplet. After round 2 droplet barcoding, cDNA from each cell was now labeled with a unique cell barcode, a combination of CB1 and CB2. cDNA libraries were then constructed using a protocol modified from the Illumina Nextera XT DNA library construction kit (FIG. 1A, FIG. 6).

### Determining the Technical Performance of BacDrop

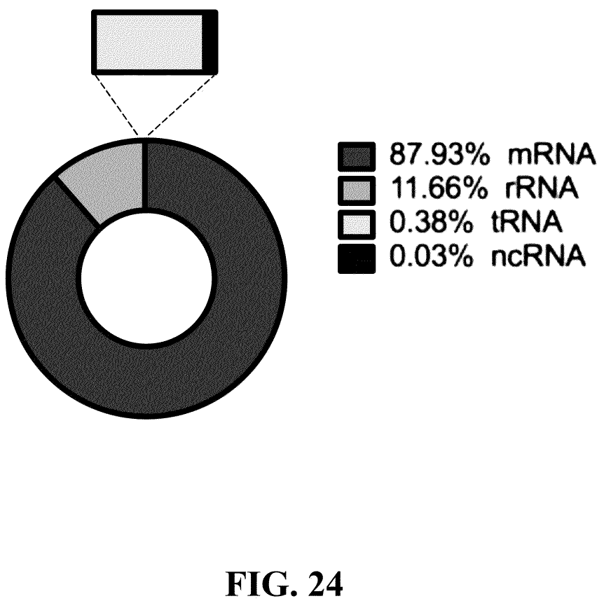

To characterize the performance of BacDrop, Applicants determined the efficiency of rRNA depletion, preservation of expression profiles despite fixation, correlation of transcriptional profiles generated by droplet library construction with bulk libraries, barcode collision rates, and generalizability to different bacterial species. After rRNA depletion, the fraction of mRNA reads increased from ~5% to 50-90% of the total aligned reads, while preserving mRNA profiles relative to non-depleted samples (R² = 0.81; FIGS. 1B & 1C; FIG. 24). In addition, Applicants confirmed that mRNA profiles were not affected by cell fixation in bulk experiments (R² = 0.97; FIG. 1D), and that BacDrop produces transcriptional profiles that are well-correlated with those generated by the traditional bulk RNA-seq method (R² = 0.91; FIG. 1E).

Applicants confirmed that the two-step barcoding strategy, which was used to enable loading of multiple cells per droplet, resulted in a minimal number of cells labelled with the same barcodes, that is the cell barcode collision rates after two rounds of barcoding. Applicants conducted round 1 plate barcoding using 384 RT primers and round 2 droplet barcoding containing on average 6 cells per droplet, though with a range of cell numbers per droplet, on a mixed population of two distinguishable species, K.pneumoniae and P.aeruginosa. Of 44,140 cells that passed the analysis threshold at the sequencing depth of ~1000 reads per cell, 42,893 cells (97.2%) had >99% of UMIs aligning to a single species, whereas the other 1,247 (2.8%) had > 1% of UMIs aligning to both species (FIG. 1F). The resulting barcode collision rate of 6.6% compares to published methods^{17,18}. Applicants performed all subsequent experiments with a target of <3 cells per droplet, which would yield a library containing maximally a million cells in each 10x channel with an even lower collision rate.

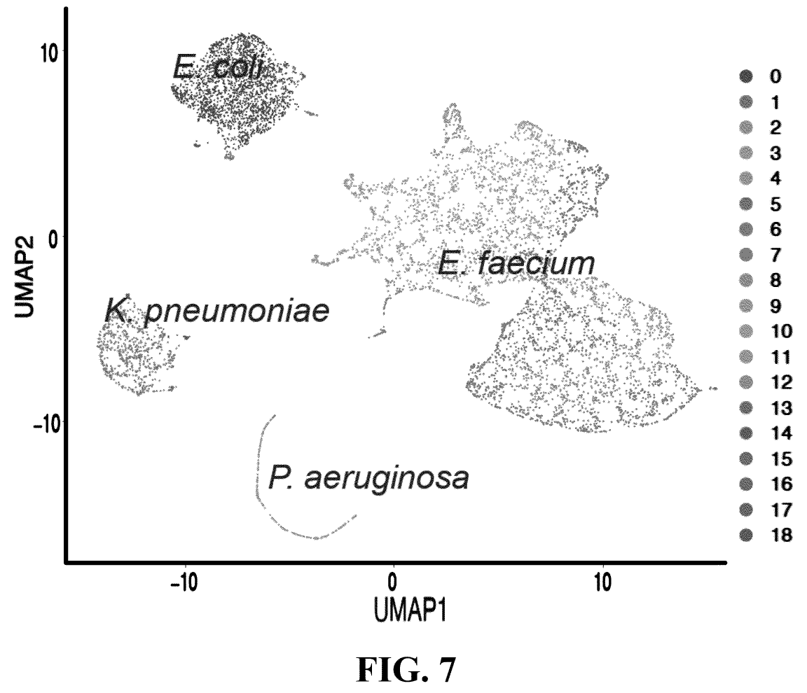

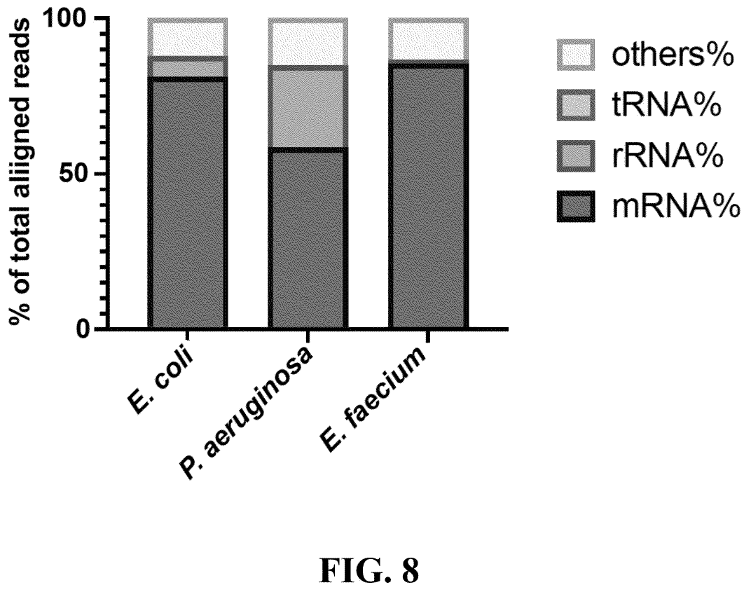

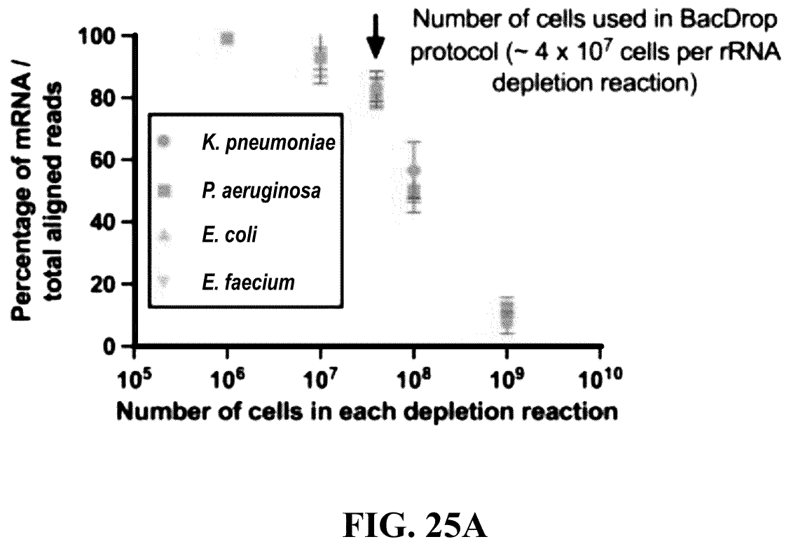

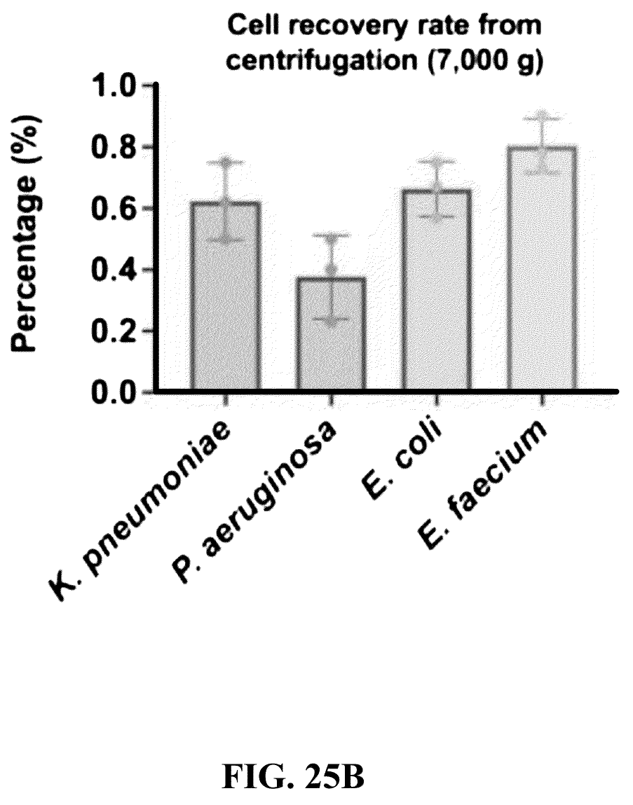

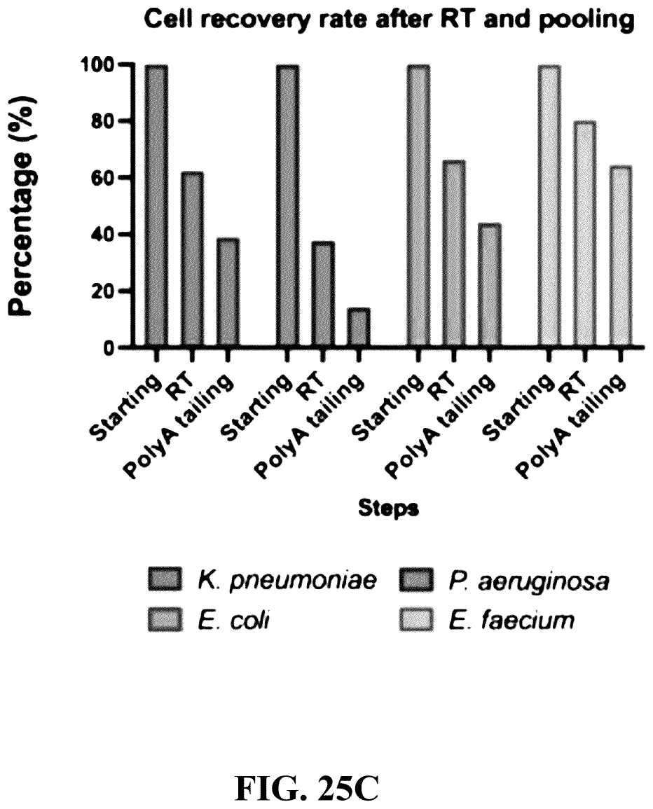

To demonstrate the generalizability of BacDrop to different bacterial species and the ability of BacDrop to differentiate among different species, Applicants applied BacDrop to the gram-negative E.coli, K.pneumoniae, P.aeruginosa, and the gram-positive E.faecium (FIG. 1G, Table 2). Applicants performed cell fixation, permeabilization, rRNA and gDNA depletion, and round 1 plate barcoding separately on individual cultures of these four species, and then mixed all four strains together into a single pool for round 2 droplet barcoding and library construction. Each of these strains were thus labeled with one unique set of CB1 (Table 1), allowing Applicants to track and validate accuracy of species identification in downstream analysis. Applicants used the standard workflow of Seurat 3 for cell clustering and subpopulation identification¹⁹. Applicants found that these 4 species were clearly distinguished in the analysis and that the RNase-H based rRNA depletion worked efficiently across all four species (FIG. 1G, FIG. 7, FIG. 8, FIG. 25A). Cells from E.faecium fell into several subpopulations distinguished by differential expression of two highly expressed housekeeping genes (ef-tu and ef-g; log2 fold change ~ 0.6) in E.faecium. Of note, the variation of cell numbers among these four species in the sequenced library was due to cell loss before round 2 droplet barcoding. Specifically, this was due to the loss of a larger fraction of P.aeruginosa cells, which are smaller, during the centrifugation steps (FIGS. 25B, 25C). For future experiments, Applicants only pooled samples right before round 2 droplet barcoding to ensure equal number of cells from each sample are loaded into the 10x channel for cell barcoding and library construction, or use of centrifugation speeds that have been optimized for each species or sample.

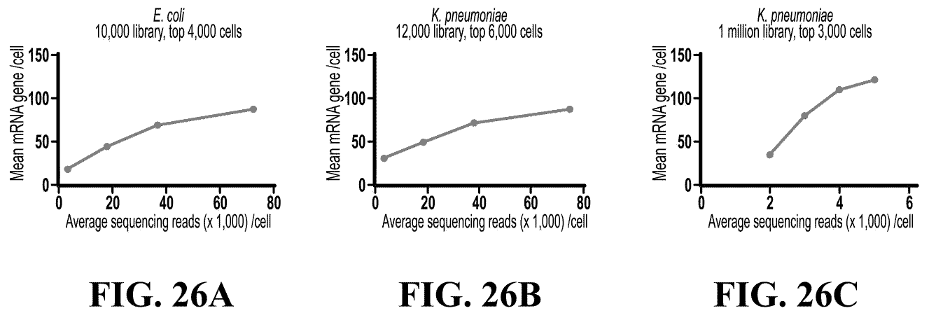

Applicants next examined the transcriptome coverage of BacDrop in E.coli and K. pneumoniae. Applicants generated two small libraries containing either ~10,000 cells of E.coli or ~12,000 cells of K.pneumoniae (FIGS. 26A, 26B). Both libraries were sequenced with ~80,000 reads per cell, and Applicants recovered roughly 4,000 or 6,000 cells, containing at least 15 mRNA genes per cell, respectively. Applicants detected the expression of ~70% genes of the E.coli genome and ~80% genes of the K.pneumoniae genome when Applicants analyzed all cells together (Table 8 and 9). At the single-cell level, Applicants detected an average of 90 and 88 mRNA genes per cell, respectively, which is comparable to other reported methods^{7,9}. Applicants also generated a large library from ~1 million K.pneumoniae cells and sequenced with ~5,000 reads per cell. Applicants recovered ~60,000 cells with at least 15 mRNA genes per cell and detected the expression of 96% genes of the whole genome when Applicants analyzed all cells together (Table 10). At the single-cell level Applicants detected an average of 30 mRNA genes per cell across all 60,000 cells. However, from this large library, the top 3,000 high-quality cells had an average of 127 mRNA genes detected per cell (FIG. 26C). Since this large library was only sequenced with ~5,000 reads per cell, Applicants expect to detect a higher number of mRNA genes per cell with increased sequencing depth, as illustrated with the smaller libraries.

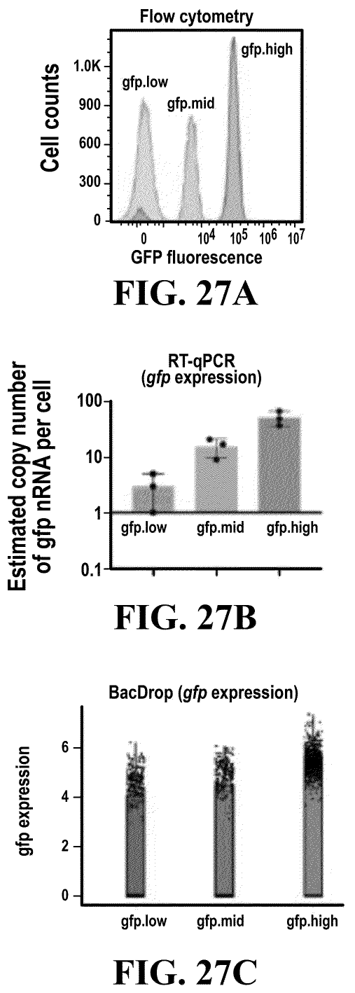

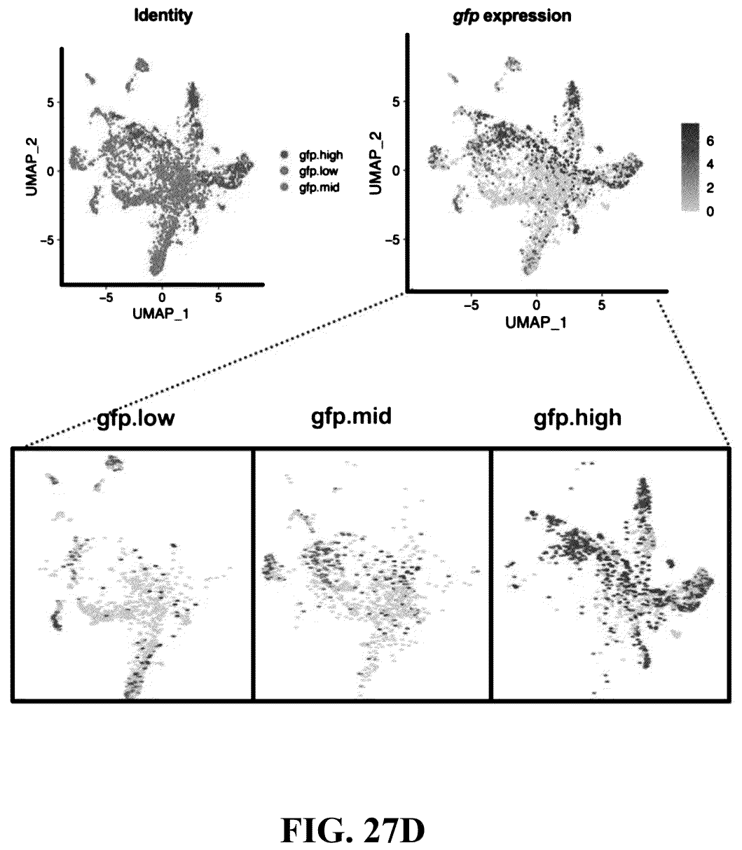

Finally, Applicants assessed BacDrop’s sensitivity to distinguish different expression levels of a gene. Applicants created a heterogeneous population containing three E.coli strains constitutively expressing gfp at different levels (Table 2). Applicants used flow cytometry to confirm the differing expression levels of gfp in these strains and estimated the mRNA copy numbers of gfp in each of these strains using RT-qPCR⁶ (FIGS. 27A, 27B). The estimated mRNA copy numbers per cell were 1-5 for the gfp.low strain, 9-30 for the gfp.mid strain, and 30-70 for the gfp.high strain. Applicants barcoded each strain with a unique set of CB1 and mixed ~3,300 cells from each strain for round 2 droplet barcoding to generate a BacDrop library. Applicants found BacDrop can distinguish cells based on their gfp expression levels. Importantly, the gfp expression levels detected via BacDrop were consistent with the flow cytometry and RT-qPCR results (FIGS. 27C, 27D), suggesting that BacDrop can reliably identify subpopulations with differentially expressed genes.

Together, these results suggest that BacDrop is a robust and reliable technology with sufficient coverage and sensitivity that can be applied across different numbers of cells and species. Applicants thus proceeded to test its capability in revealing biological heterogeneity in a single K. pneumoniae clinical isolate, using these experiments to additionally demonstrate BacDrop’s reproducibility.

### Validating BacDrop’s Ability to Identify Subpopulations of a Single Bacterial Isolate

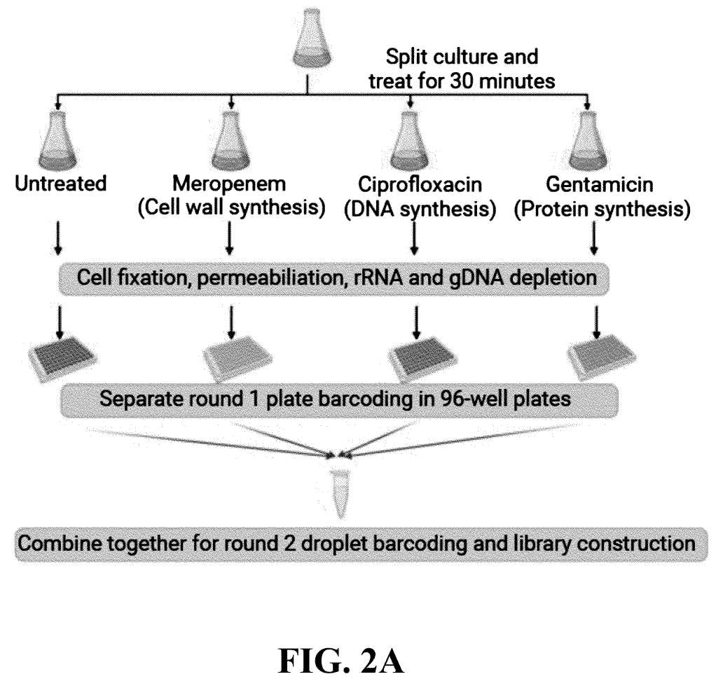

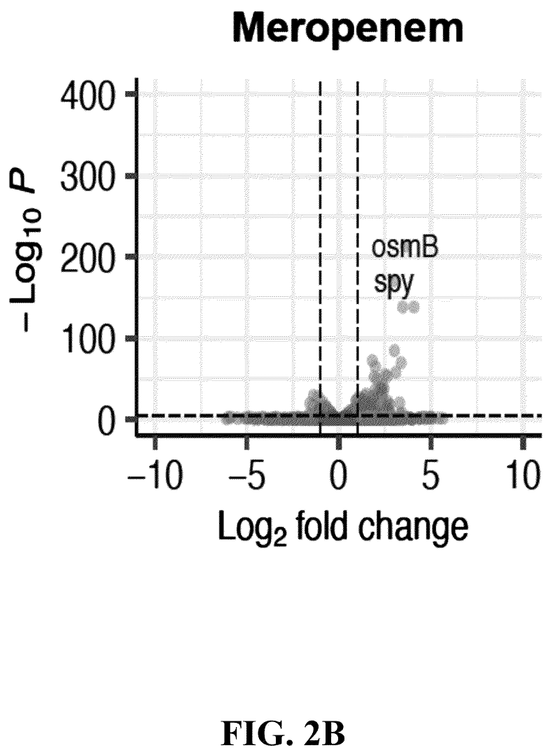

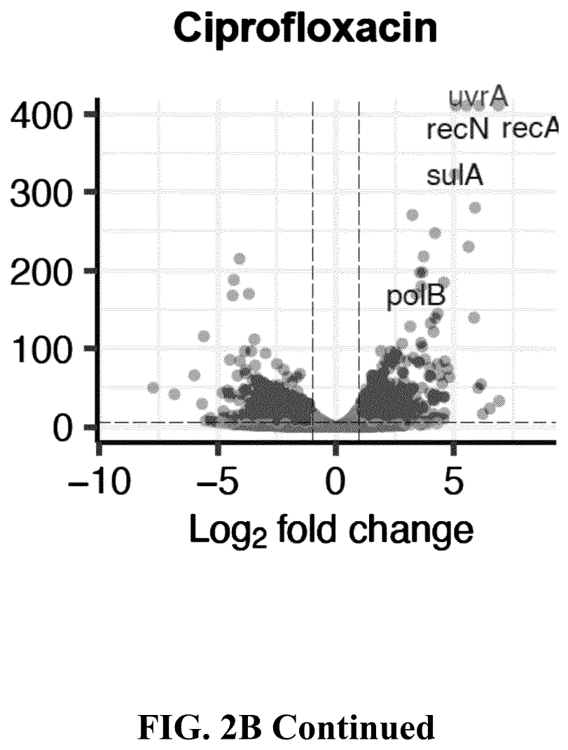

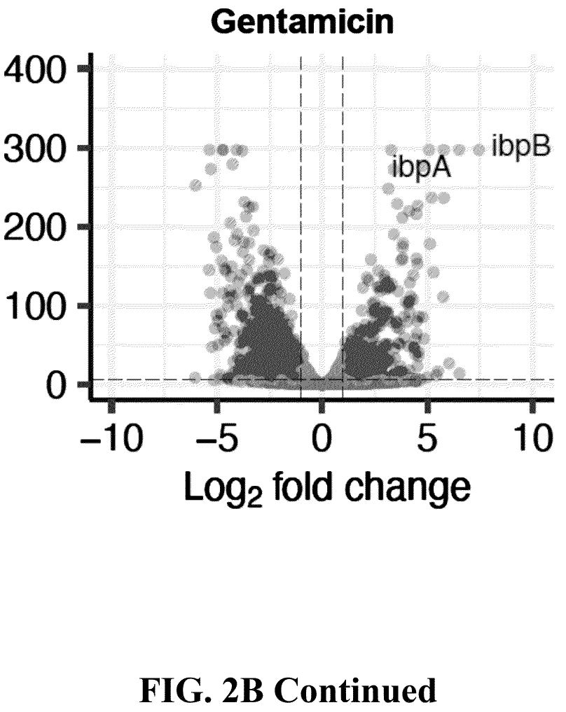

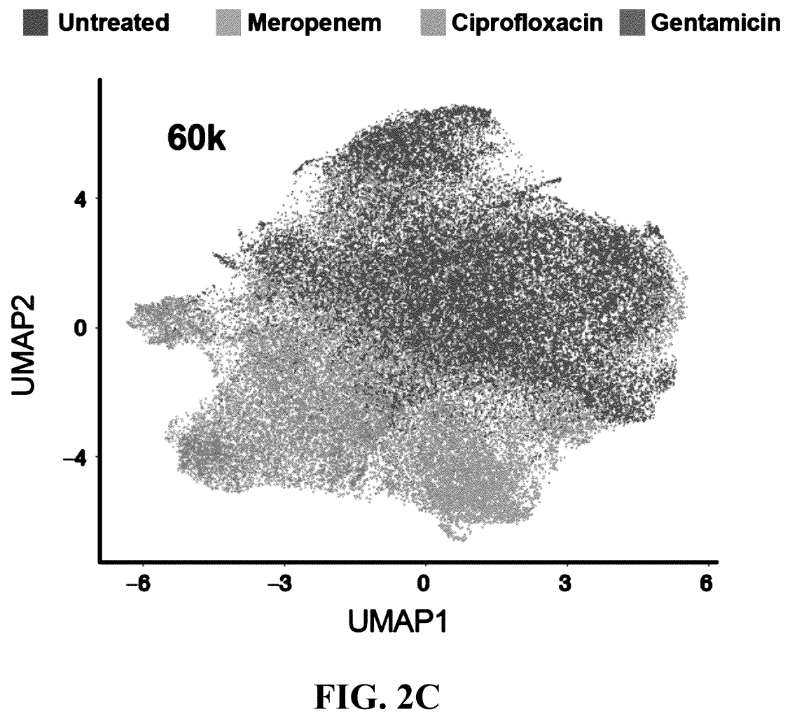

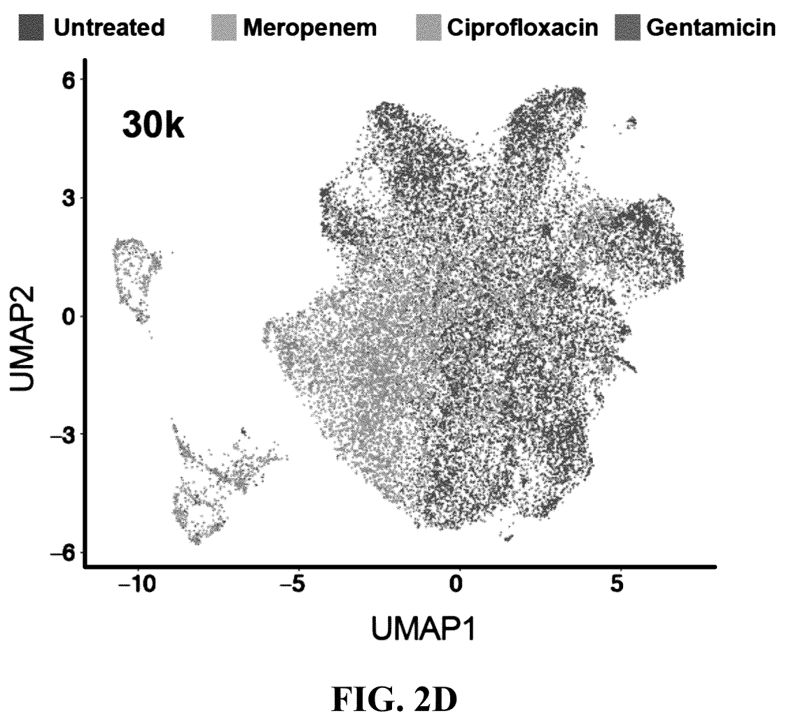

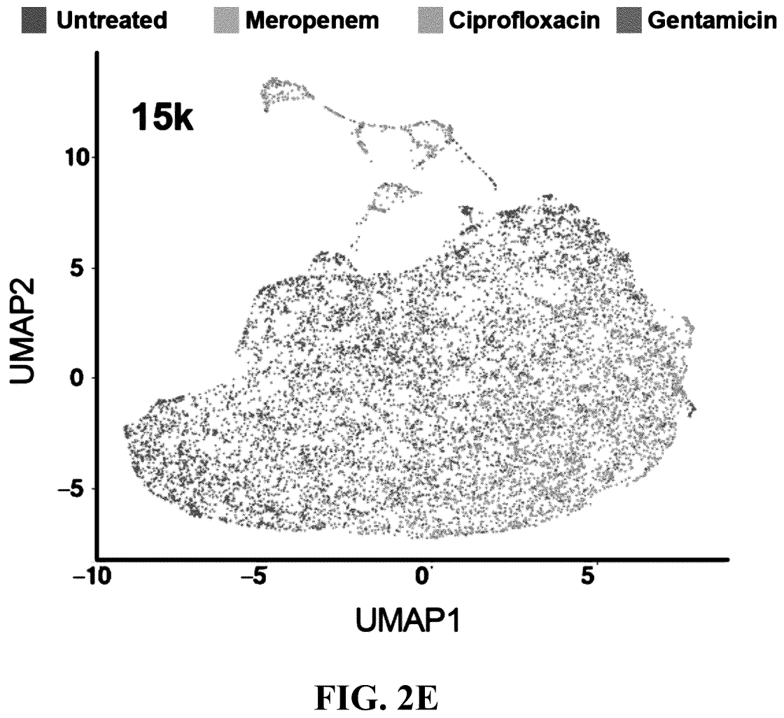

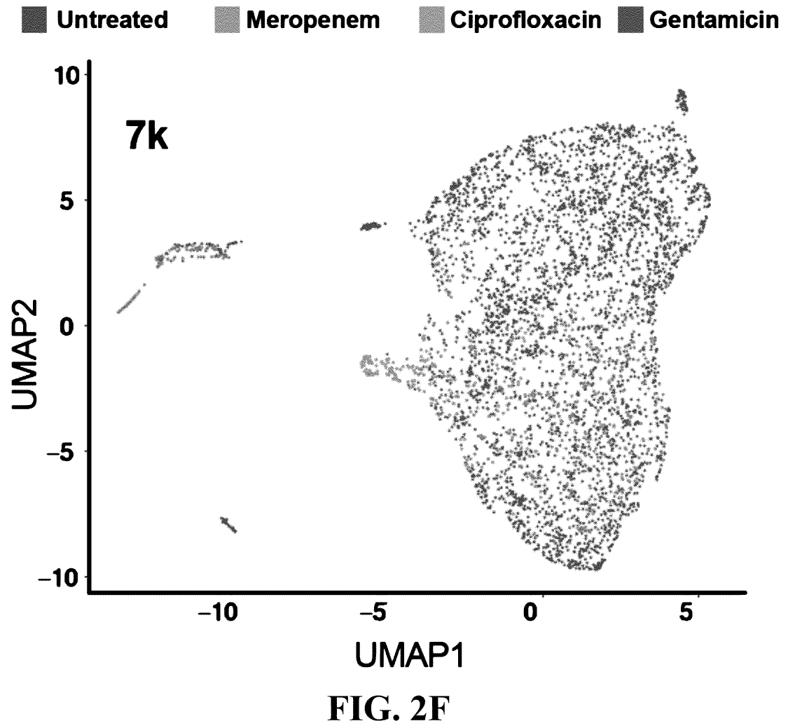

To validate that BacDrop could reproducibly identify subpopulations of cells of the same isolate, Applicants applied BacDrop to the antibiotic-susceptible clinical isolate, K. pneumoniae MGH66 in the absence and presence of antibiotic perturbations (FIG. 2A, Table 2). Applicants split a MGH66 culture (OD₆₀₀ ~0.2) into 4 identical cultures and treated 3 of them for 30 minutes with 3 antibiotics with different mechanisms of action, including inhibition of cell-wall synthesis (meropenem), DNA synthesis (ciprofloxacin), and protein synthesis (gentamicin), the fourth culture were left untreated (see methods for details). Bulk RNA-seq on the samples collected using the same treatment schemes revealed distinct cellular responses to each antibiotic relative to untreated control. As expected (FIG. 2B), ciprofloxacin induced genes involved in SOS response, e.g., recA., while gentamicin treatment induced a group of heat shock chaperone proteins, e.g., ibpB. In contrast, at 30 minutes, minimal transcriptional responses were observed in bulk from meropenem treatment, under the condition used (OD₆₀₀ ~ 0.2, meropenem concentration 2 µg/mL), which is consistent with previous observations²⁰ (FIG. 2B).

To validate BacDrop, Applicants applied round 1 plate barcoding to each of these 4 samples separately using one of 4 distinct sets of CB1 (96 different CB1s corresponded to each sample, Table 1). The distinct CB1 set identities allowed Applicants to confirm the original identity of these 4 samples and associate them with the corresponding antibiotic exposure. Applicants then mixed all cells from the different treatments for round 2 droplet barcoding and library construction (FIG. 2A). Each of the replicate libraries contained roughly one million cells. Applicants sequenced Replicate 1 with ~5 billion paired-end reads (~5,000 reads per cell) and Replicate 2 with ~3 billion paired-end reads (~3,000 reads per cell).

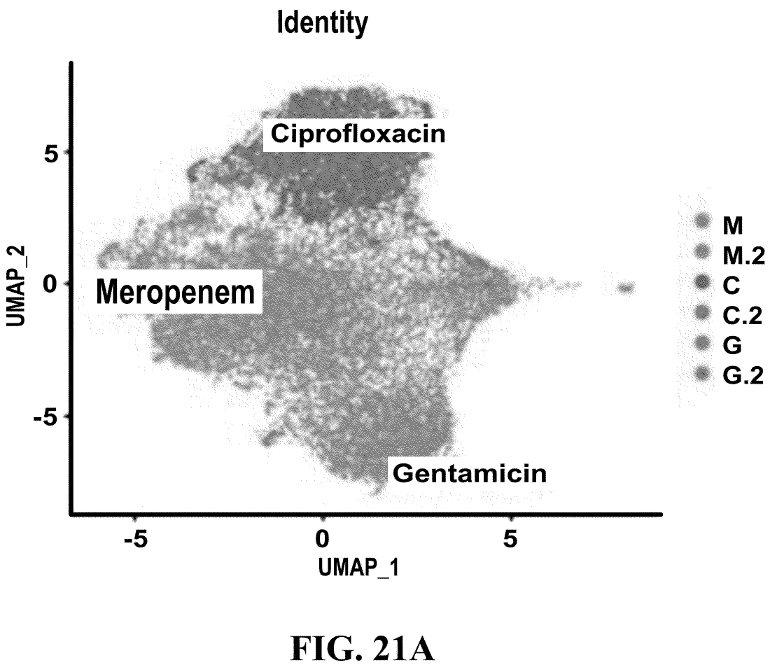

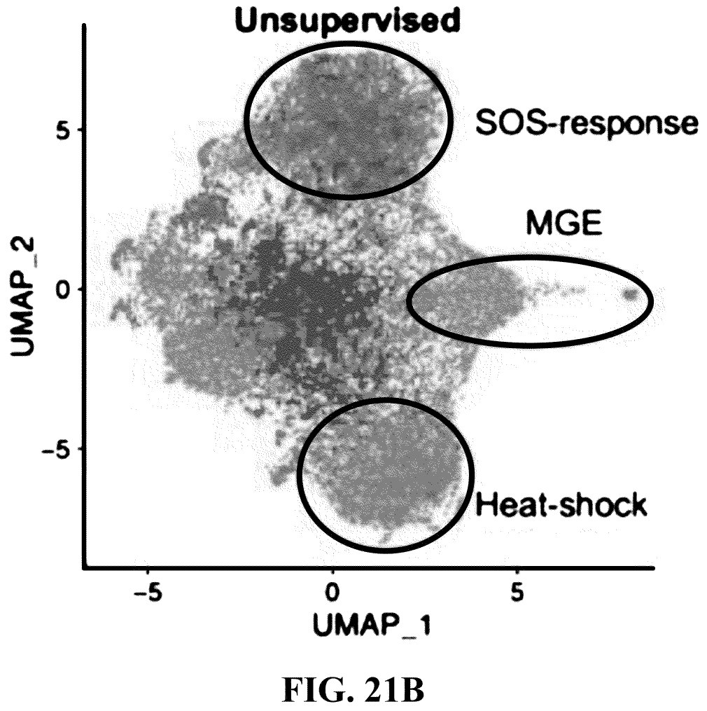

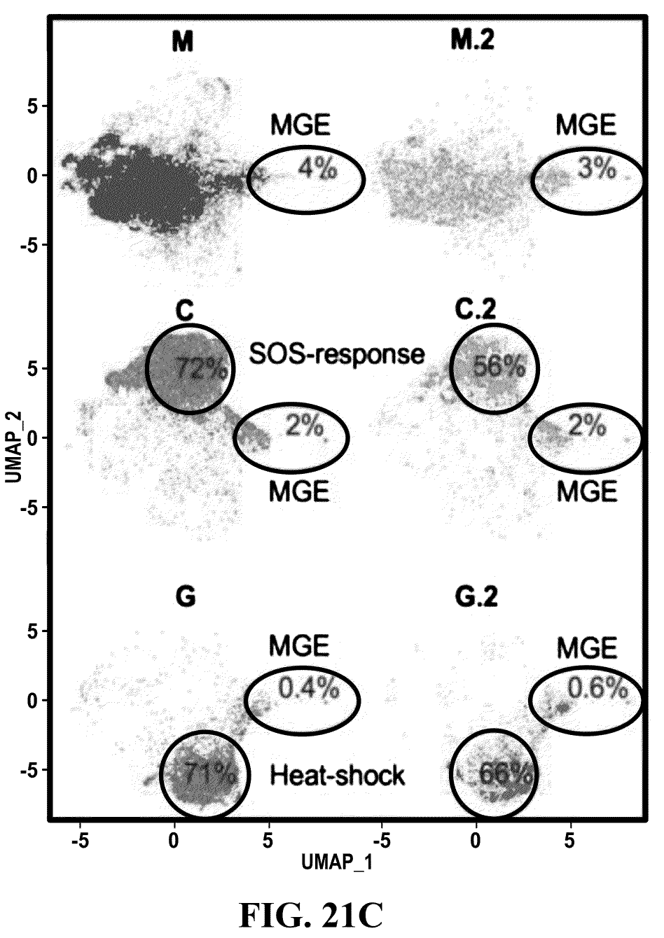

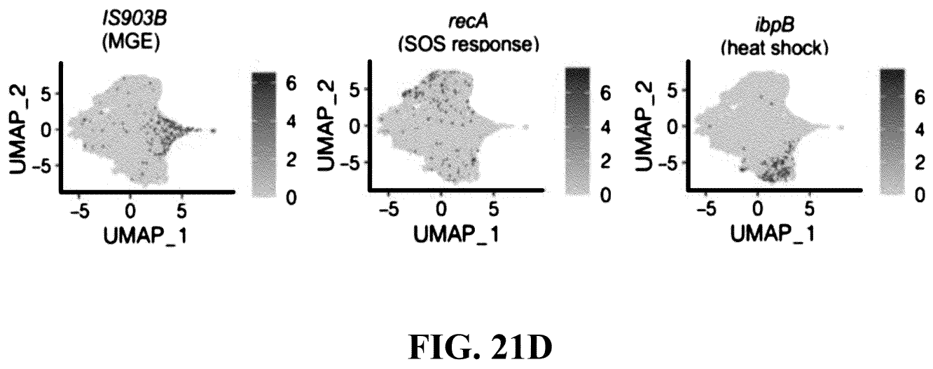

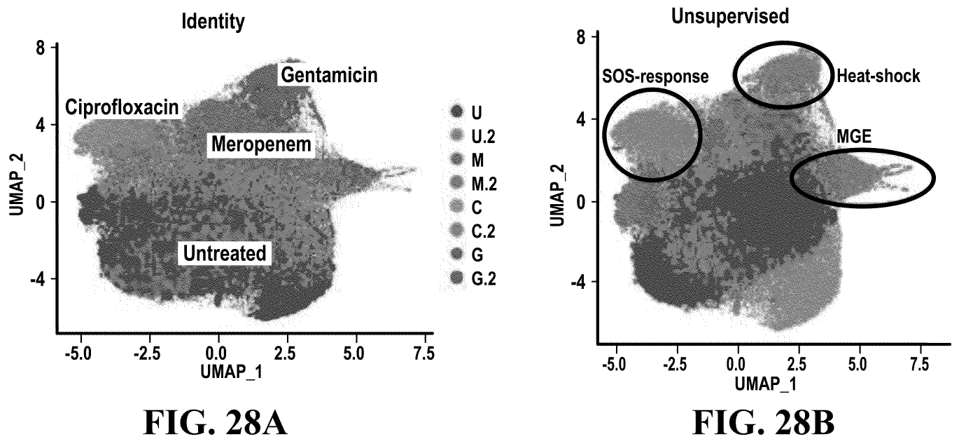

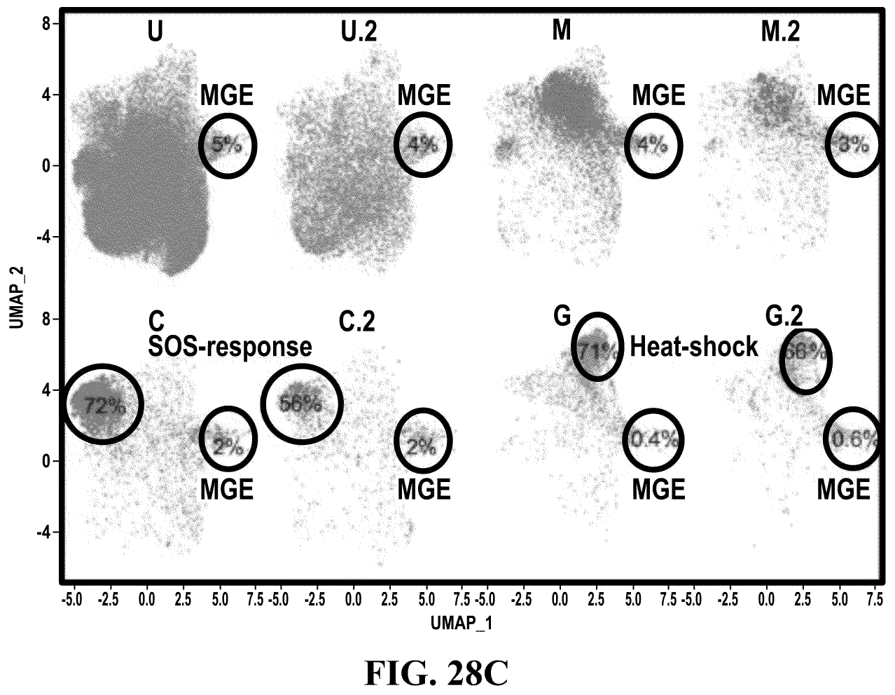

To confirm BacDrop’s robustness and reproducibility, Applicants compared the two replicate experiments, each involving 4 samples (3 antibiotic treated and 1 untreated). For the depth of sequencing performed for each experiment, Applicants obtained ~80,000 cells with ≥15 mRNA genes per cell, with an average of ~30 unique mRNA genes detected per cell. No strong batch effect was observed between the replicates. Treatment with the different antibiotics, ciprofloxacin, gentamicin, or meropenem, resulted in cells clustering based on their treatment conditions, (FIGS. 21A-21D). There was some overlap between the meropenem-treated and untreated samples (FIG. 28), consistent with their bulk RNA-seq results (FIG. 2B). Across both replicates, 56%-72% of the ciprofloxacin-treated cells belonged to the SOS-response cluster, while 66%-71% of the gentamicin-treated cells belonged to the heat-shock response cluster, with good reproducibility across the two replicates (FIG. 21B).

Besides the clusters driven by SOS response and heat-shock response, another cluster showed significantly higher expression of a mobile genetic element (MGE), an IS903B transposase (FIGS. 21B-D). This MGE subpopulation was observed in all 8 samples regardless of the treatment, suggesting the presence of this MGE population is a robust, reproducible phenomenon. Together, these experiments confirmed that BacDrop could effectively and reproducibly identify population heterogeneity.

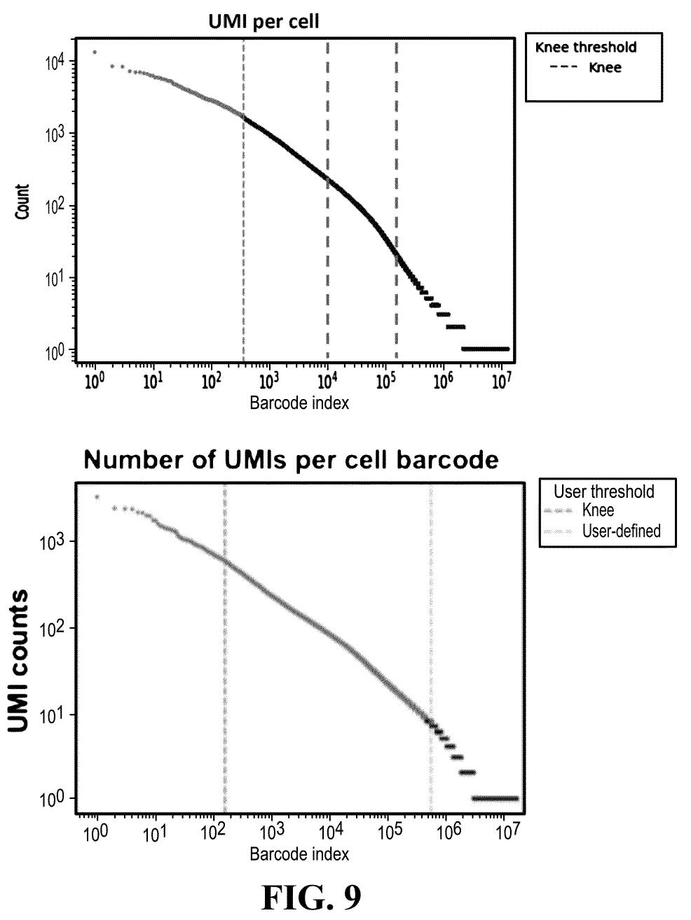

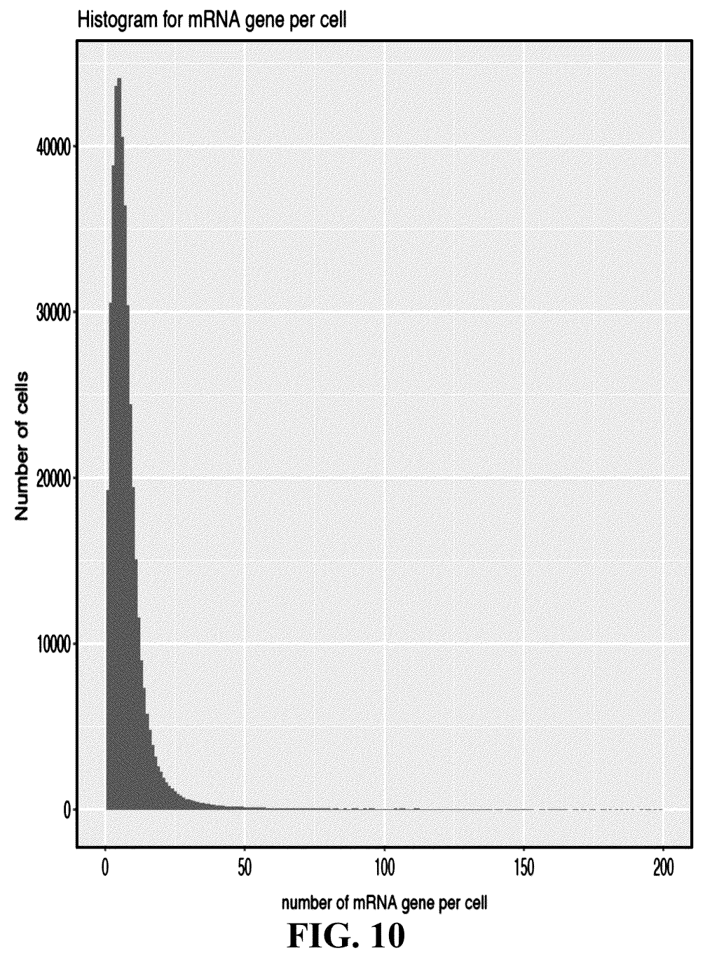

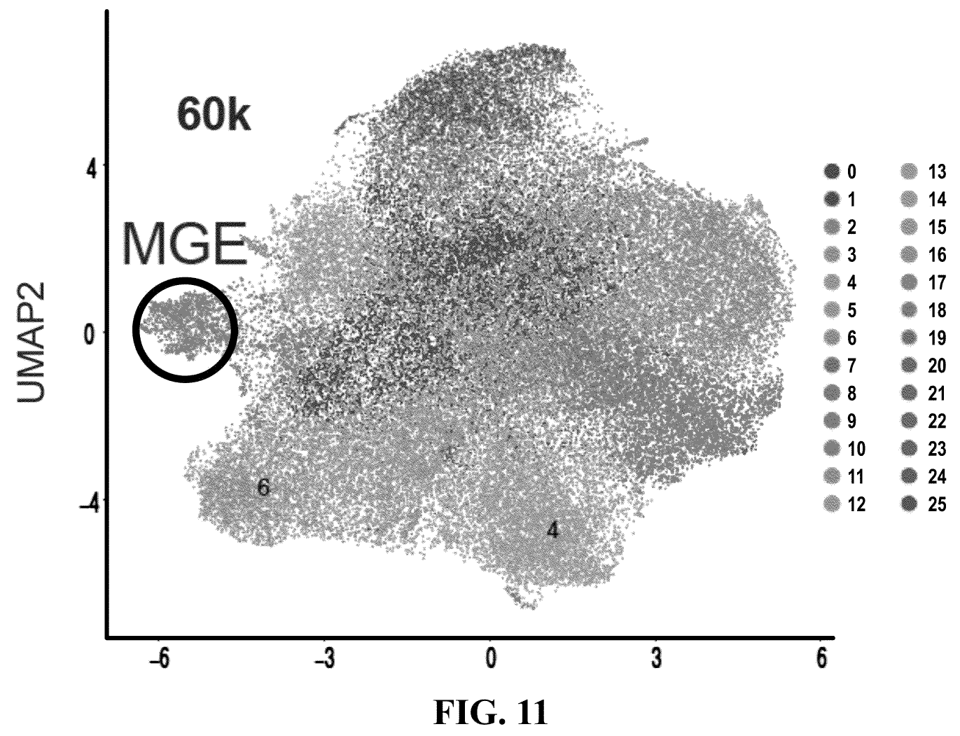

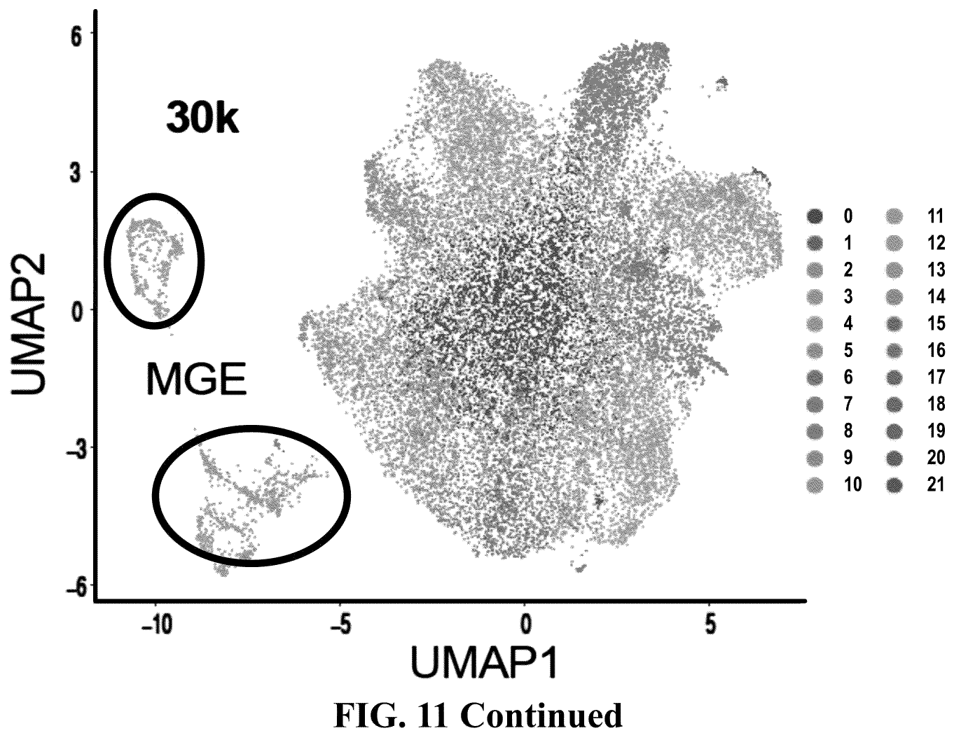

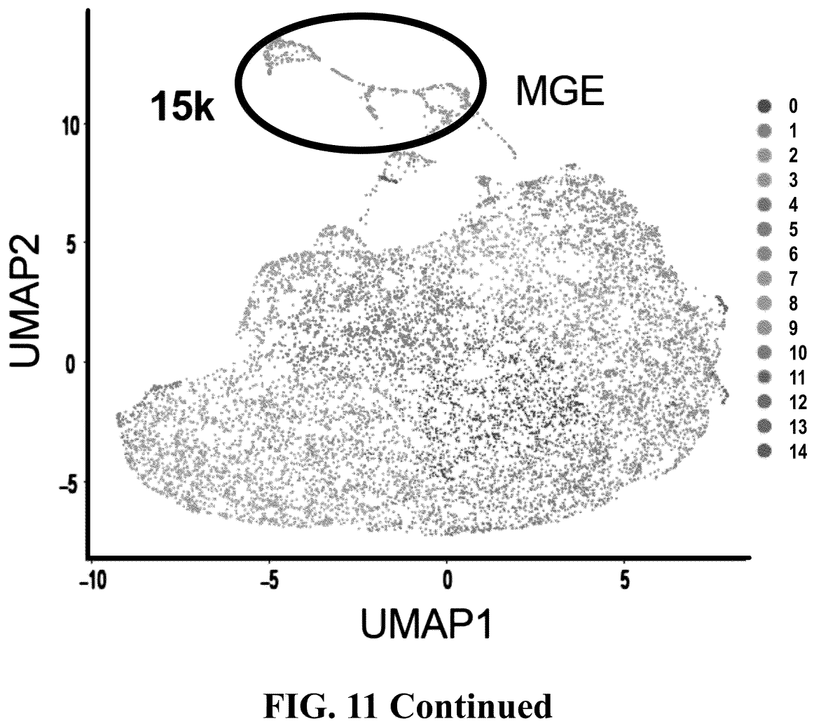

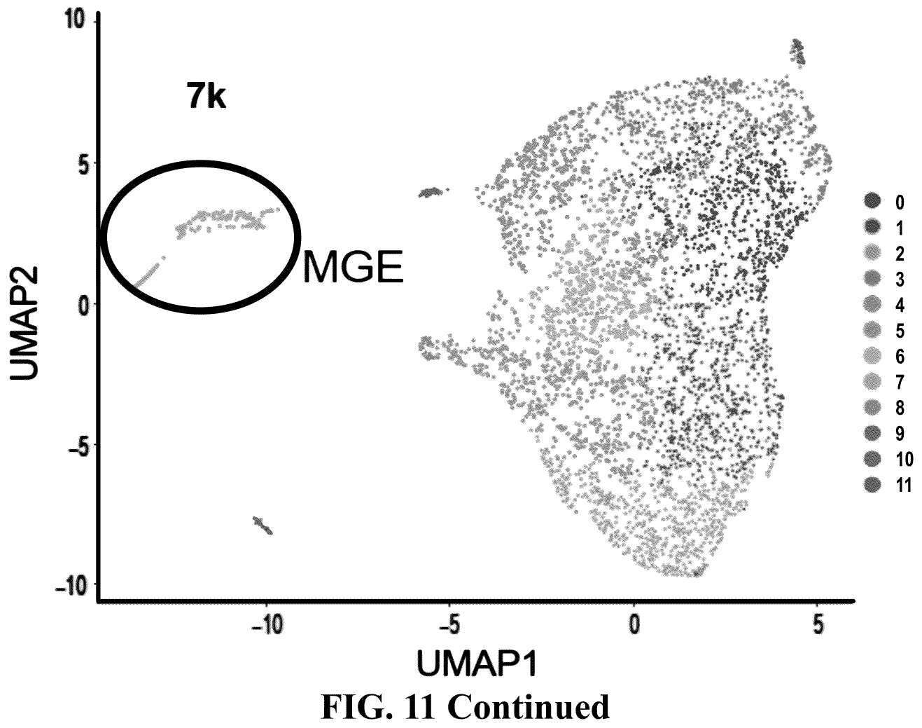

Using UMI-Tools²¹, Applicants extracted reads from cell barcodes with more than 10 unique molecular identifiers (UMIs) detected (FIG. 9), which constituted ~74% of the total sequencing reads. Applicants then aligned the extracted sequences and quantified numbers of mRNA UMIs and genes in each cell. Applicants found ~350,000 cells containing 1 to 9 mRNA genes, ~90,000 cells containing 10-14 mRNA genes, and ~60,000 cells containing ≥ 15 mRNA genes (FIG. 10). For cells which had the greatest sequencing depth, for example, the top 2000 cells, Applicants obtained a median number of 104 mRNA genes per cell. Applicants analyzed the ~60,000 cells that contained ≥15 mRNA genes, which had a median of ~30 mRNA genes detected, and the result showed a clear distinction among these 4 samples, especially for the ciprofloxacin and gentamicin treated samples (FIG. 2C, FIG. 11), confirming that BacDrop could effectively identify population heterogeneity. As expected, however, the ability to distinguish these samples depended on the numbers of cells analyzed. Applicants down-sampled 50%, 25%, and 10% cells from each sample and repeated the analyses, resulting in a total number of ~30,000, 15,000, and 7,000 cells. As the numbers of cells in the analysis decreased, distinctions between these four samples became less clear (FIGS. 2C - 2F, FIG. 11). When ~30,000 cells were included, the gentamicin treated cells and ciprofloxacin treated cells still stood out as distinct cell clusters, driven by either SOS response genes (ciprofloxacin-treated cells) or heat-shock chaperone proteins (gentamicin-treated cells), whereas the majority of meropenem treated cells clustered with the untreated cells. In contrast, when only ~7,000 cells were included, no clear clustering was observed except for the ciprofloxacin-treated sample from all other cells. Together, these results suggest that higher throughput, even on the order of tens of thousands of cells, facilitates cell classification and subpopulation identification in bacterial scRNA-seq experiments.

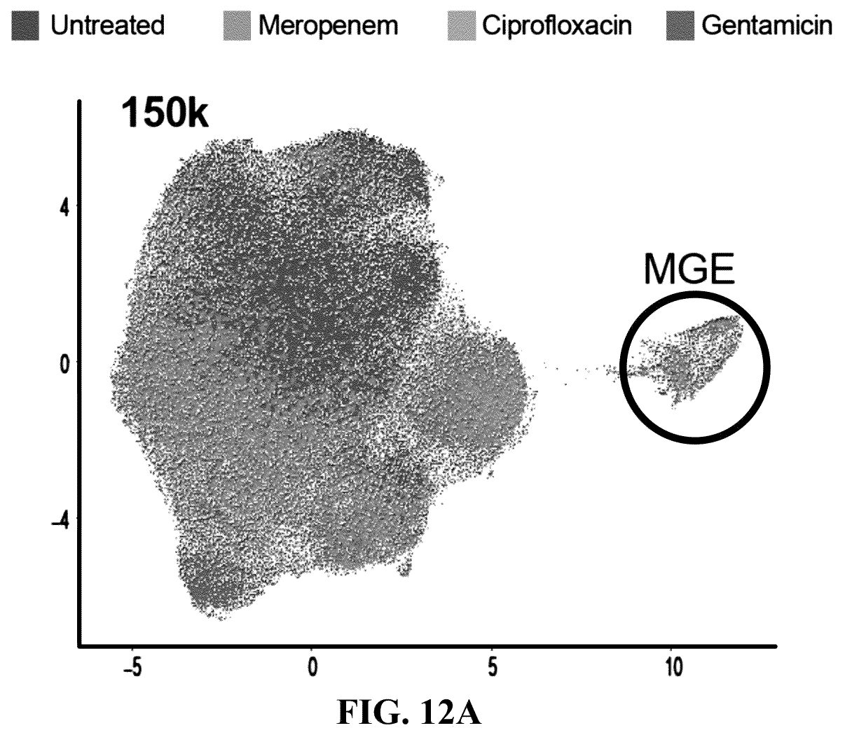

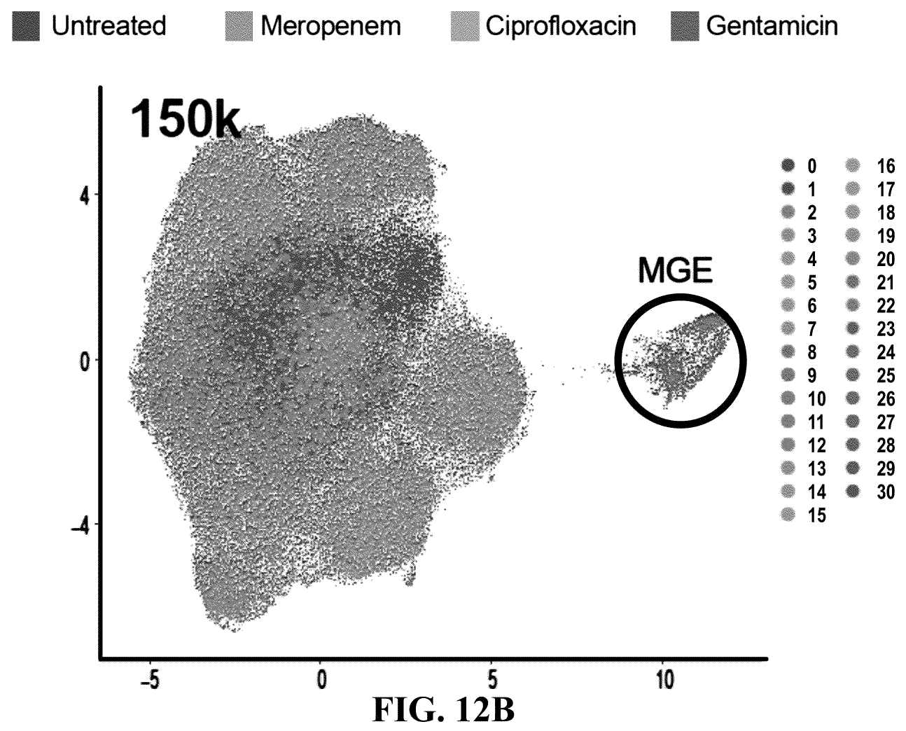

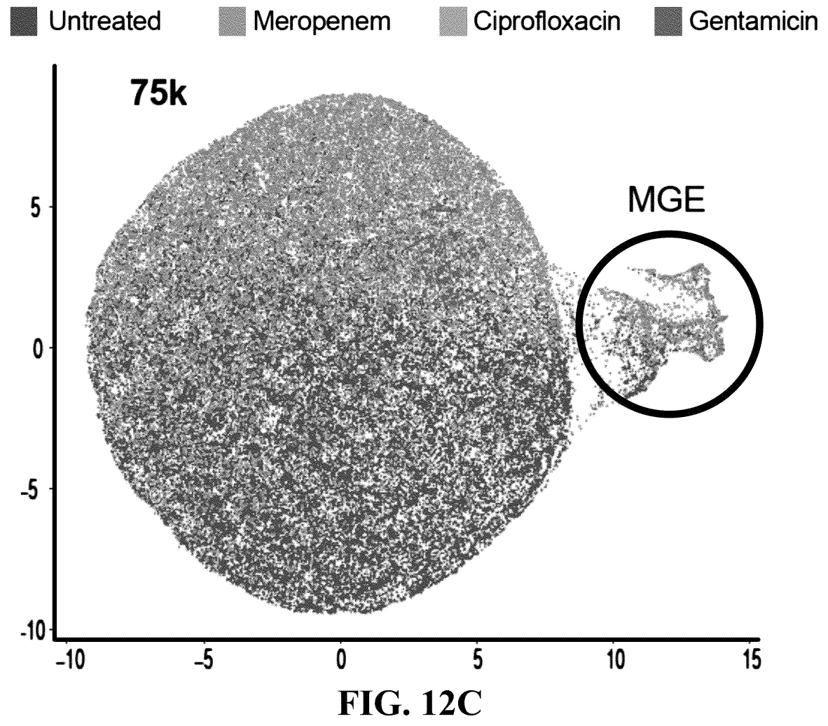

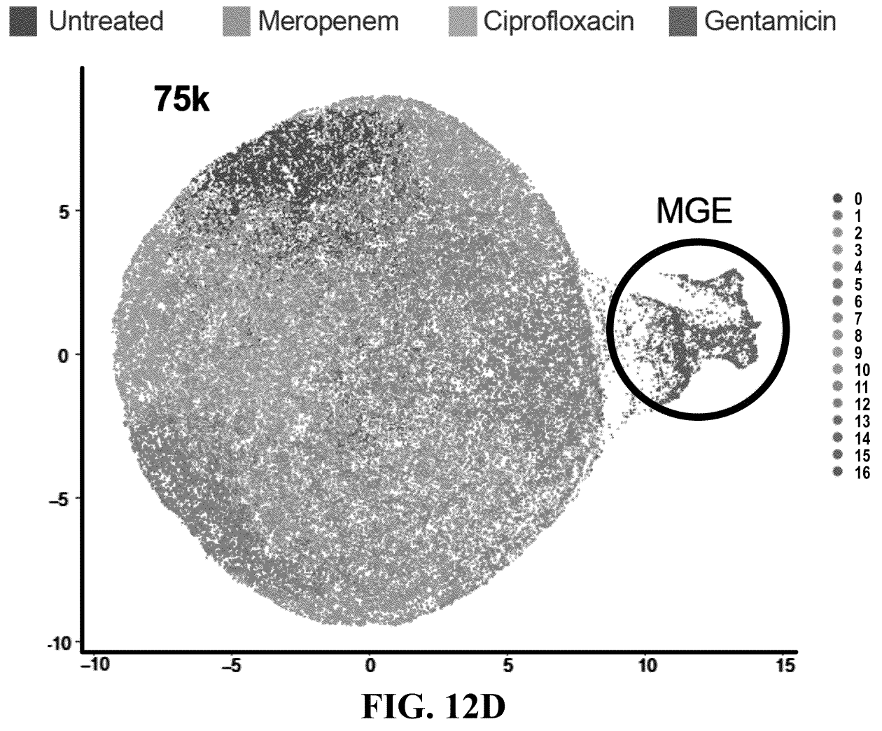

Of note, Applicants also performed this same analysis using all cells with ≥10 mRNA genes detected, rather than ≥15. Applicants found equally good clustering of these four samples (FIGS. 12A, 12B) when all ~150,000 cells were included. However, when Applicants down-sampled to ~75,000 cells (50%), the separation of these four samples was not as good (FIGS. 12C & 12D).

### BacDrop Reveals Within Population Heterogeneity With Subpopulations Driven Largely by the Expression of Mobile Genetic Elements (MGEs)

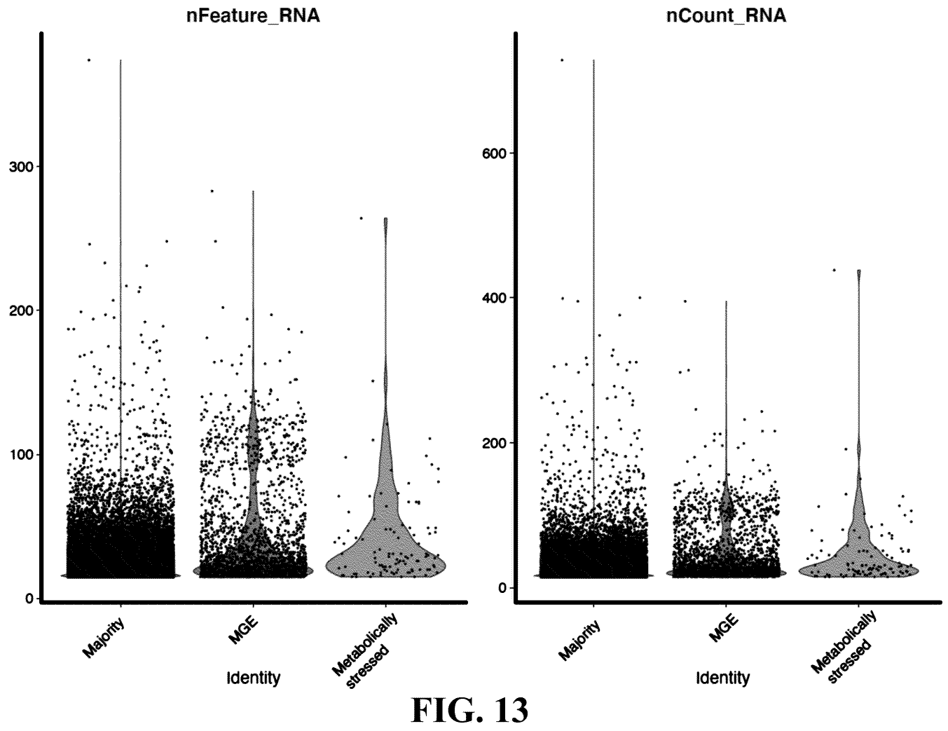

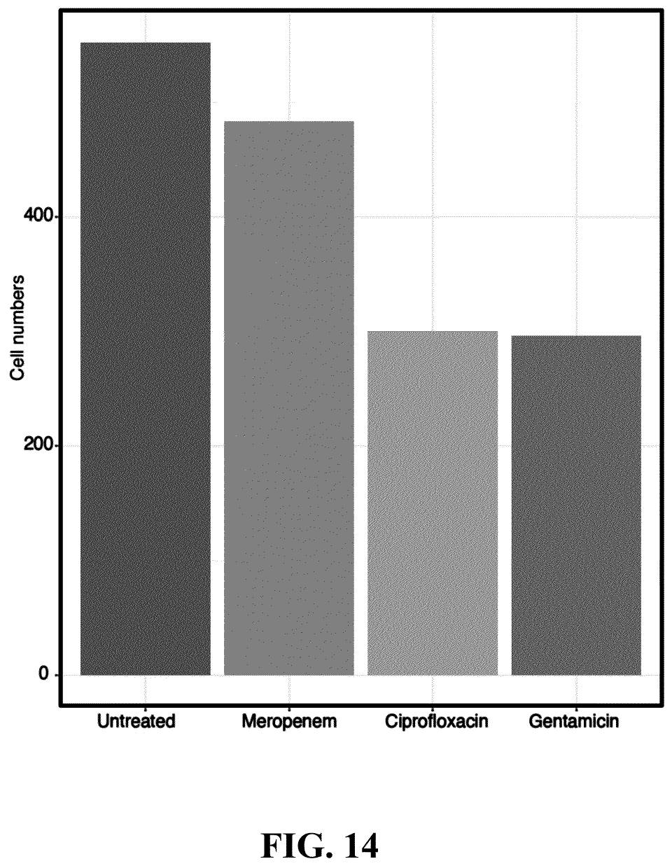

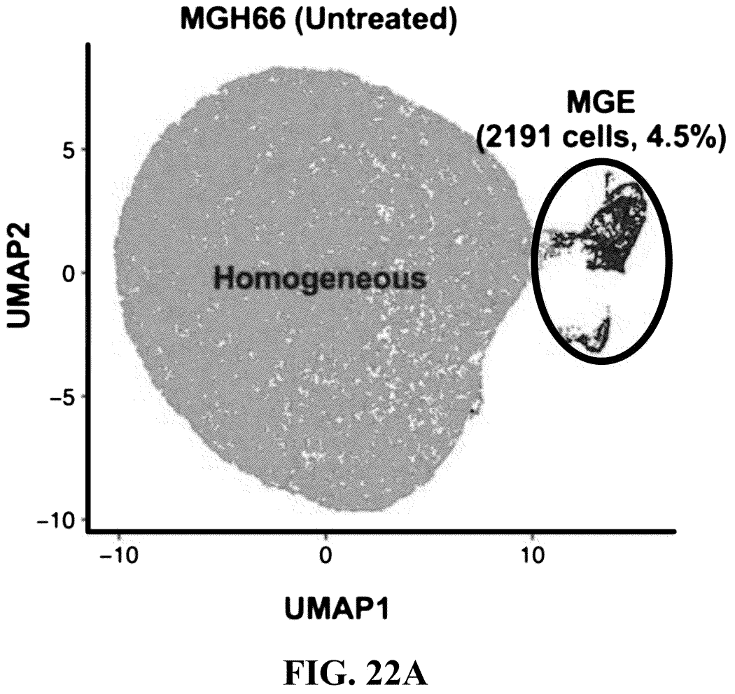

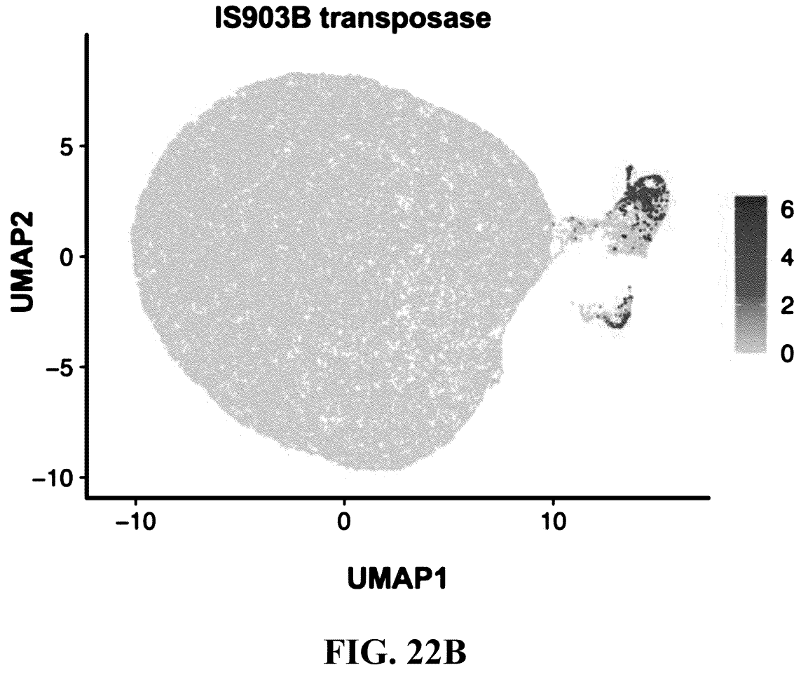

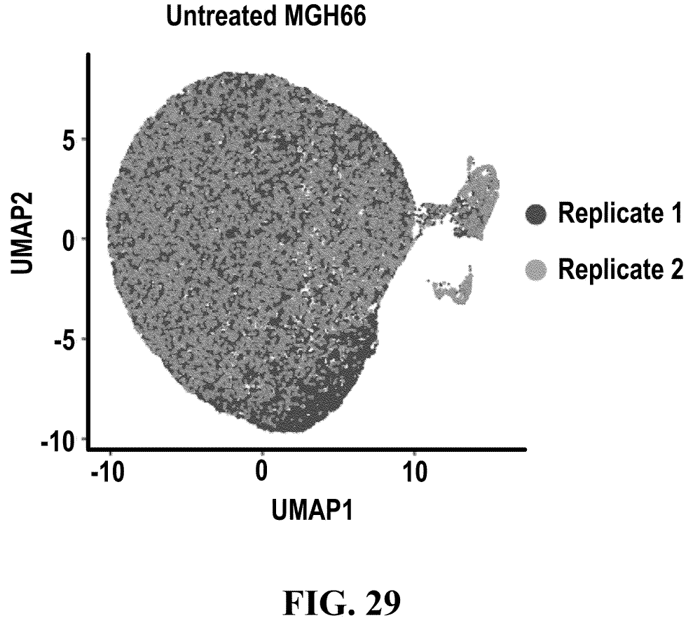

Applicants next analyzed the untreated culture of MGH66 at the single cell level to understand if it contained any previously unrecognized within population heterogeneity in transcriptional states. From this untreated condition Applicants recovered ~50,000 cells with at least 15 unique mRNA genes detected in each cell, and Applicants identified two major subpopulations in both replicates using an unsupervised clustering approach (see Methods; FIG. 22A, FIG. 29). While the majority of cells fell into one major homogenous subpopulation, 2,191 cells (~4.5%) fell into the MGE subpopulation driven by IS903B (FIG. 22B), which has 83 copies in MGH66 genome. In an analysis of ~40,000 cells with ≥15 or more unique mRNA genes detected in each cell, Applicants identified three subpopulations (FIG. 2A, FIG. 13) using an unsupervised clustering approach (see methods). While the majority of cells (~92%) fell into one major subpopulation, one smaller population, containing 2,263 cells (5.7%), was distinguished by the high-level expression of a mobile genetic element, an IS903B family transposase, which has 83 copies in MGH66 genome. Due to the limitation of the alignment program, Applicants cannot determine if the high-level expression of IS903B transposase in this subpopulation was caused by specific copies or all of the copies existing in the genome. Nevertheless, the overall expression level of IS903B in this subpopulation was hundreds of times higher than its expression in the rest of the population. Of note, this same MGE subpopulation was observed in the other 3 samples treated with different antibiotics (FIG. 14), suggesting the presence of this MGE population is a robust, reproducible phenomenon.

Previously Applicants had functionally shown that MGH66 and several other K. pneumoniae isolates have high-level transposon insertional mutagenesis activity, which contributes to their high frequencies of carbapenem resistance acquisition²²; however, bulk RNA-sequencing had failed to show elevated expression of any transposon genes in these strains. Here, the existence of this subpopulation with high-level transposon activity provides a possible explanation for the strain’s elevated carbapenem-resistance frequencies, with resistance likely emerging from this small subpopulation.

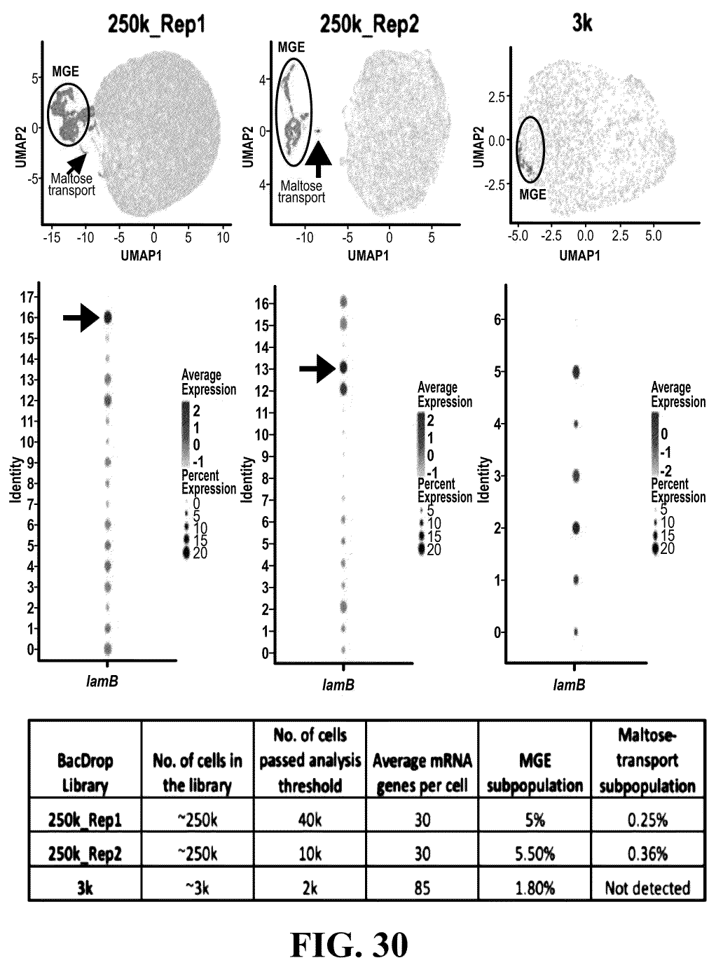

To examine the robustness of the MGE subpopulation in the datasets and to compare the relative values of deeper sequencing of fewer cells versus more shallow sequencing of more cells, Applicants analyzed the untreated samples in Replicate 1 and Replicate 2 libraries separately, along with a smaller BacDrop library containing ~3,000 cells of MGH66 similarly collected. Applicants sequenced this smaller library more deeply to obtain 80,000 reads per cell with recovery of ~2,000 cells with at least 15 mRNA genes per cell, and collectively an average of 85 mRNA genes detected per cell. Analysis of this smaller library detected the same MGE population identified in the larger cell population, albeit less distinctly (FIG. 30). In contrast, both the replicates of the larger libraries, even when sequenced only to a depth to obtain ~30 mRNA genes per cell, identified an additional small subpopulation (0.25% - 0.36%) featuring high-level expression of maltose transport genes that was not identified in the smaller library, despite its deeper sequencing (FIG. 30). Together, these results show that analyzing larger numbers of cells can help to reveal heterogeneity within a population. They also highlight BacDrop’s versatility and ability to be applied to the study of both large and small cell numbers, depending on the biology of interest.

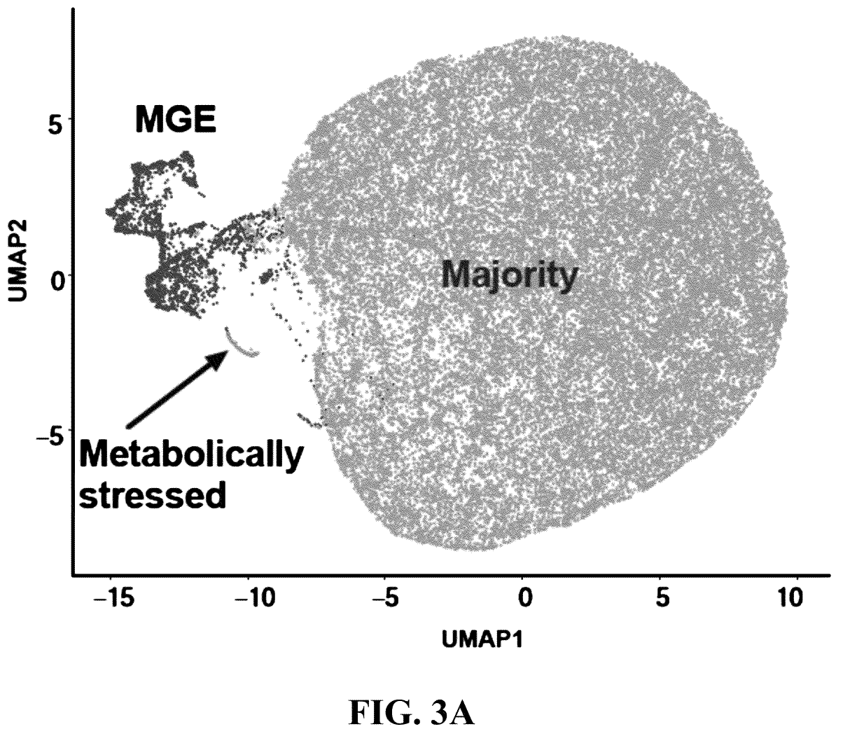

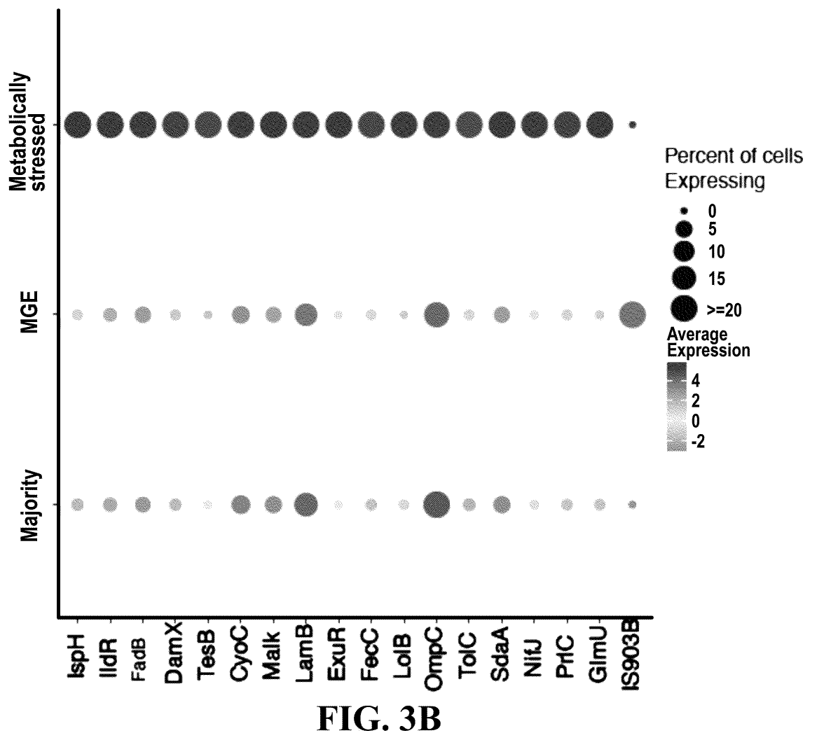

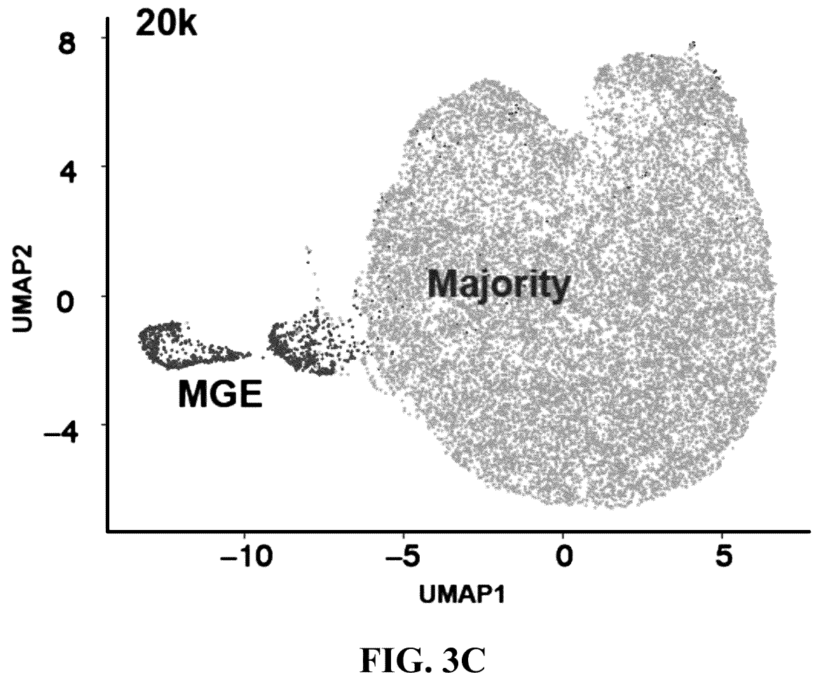

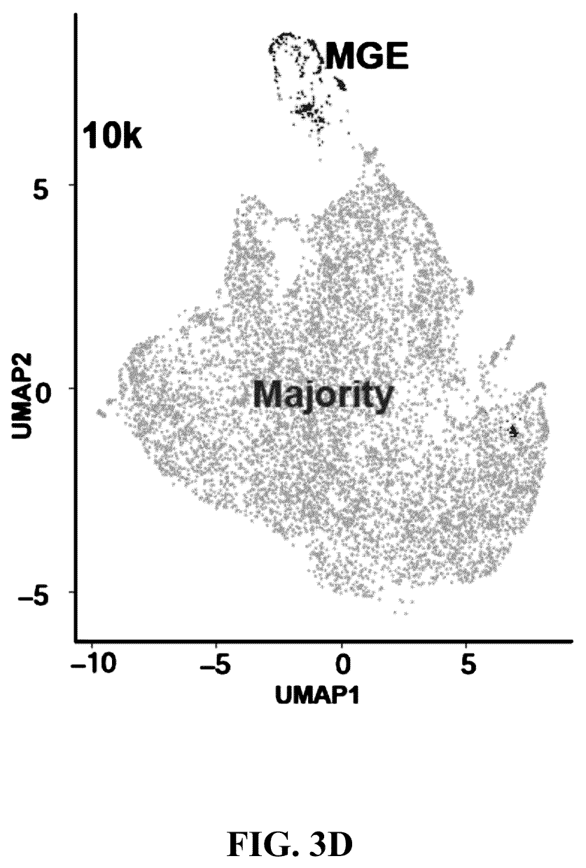

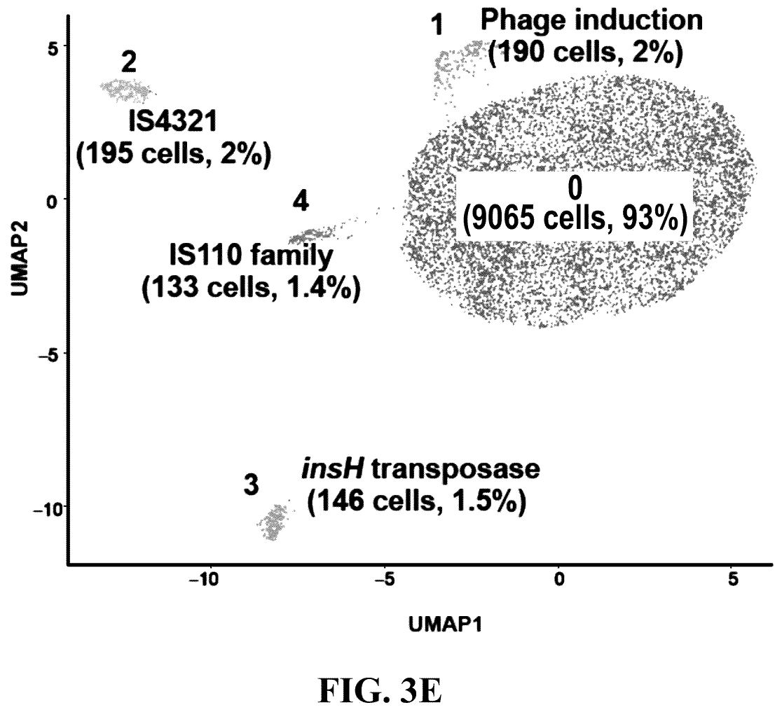

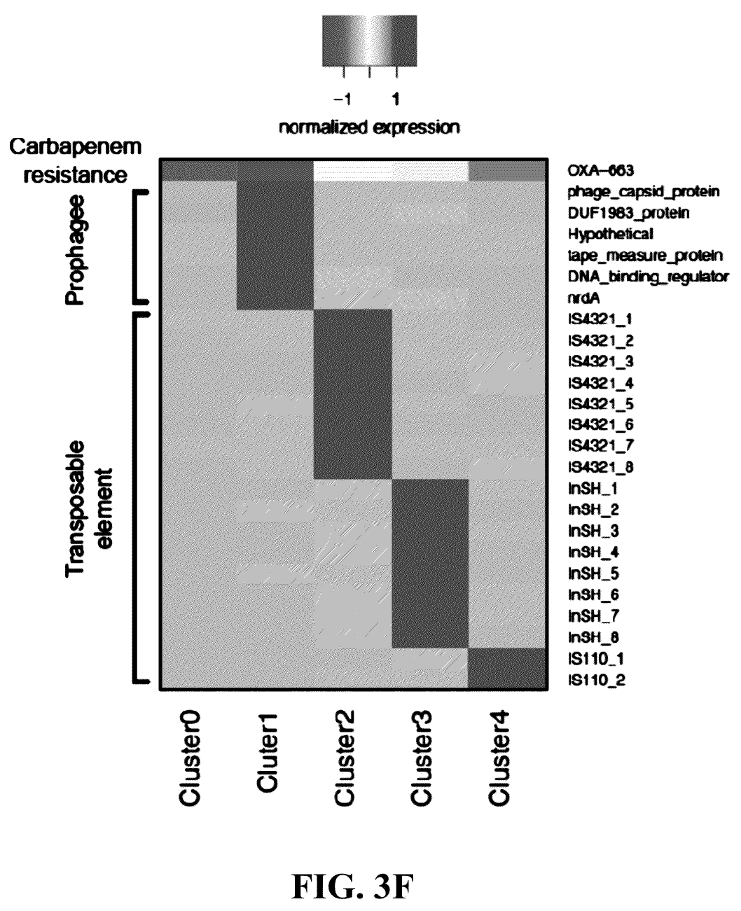

An even smaller subpopulation comprised of 103 cells (0.25%) showed a distinct metabolic state from the rest of the population (FIGS. 3A & 3B). This group of cells had significant up-regulation of genes involved in the stringent response (i.e., ispH) and starvation response, including genes involved in biofilm formation (lldR), filamentation (damX), and fatty acid degradation (fadB), as well as many nutrient transport genes, including genes involved in maltose transport (malK and lamB), ion transport (fecC), lipoprotein transport (lolB), and the major porin gene ompK36 which plays a role in nutrient uptake. In addition, Applicants found the major efflux pump, tolC, is also significantly up-regulated. These observations indicate that this subpopulation might be undergoing metabolic stress and actively importing nutrients. Of note, this small metabolically stressed population was sufficiently rare that 40,000 cells were required in order to detect it. Down-sampling to 50% or 25% of the total cells rendered this subpopulation undetectable, whereas the larger MGE-driven population could still be detected (FIGS. 3C & 3D). This result suggests that high throughput nature of BacDrop is advantageous for detecting rare populations.

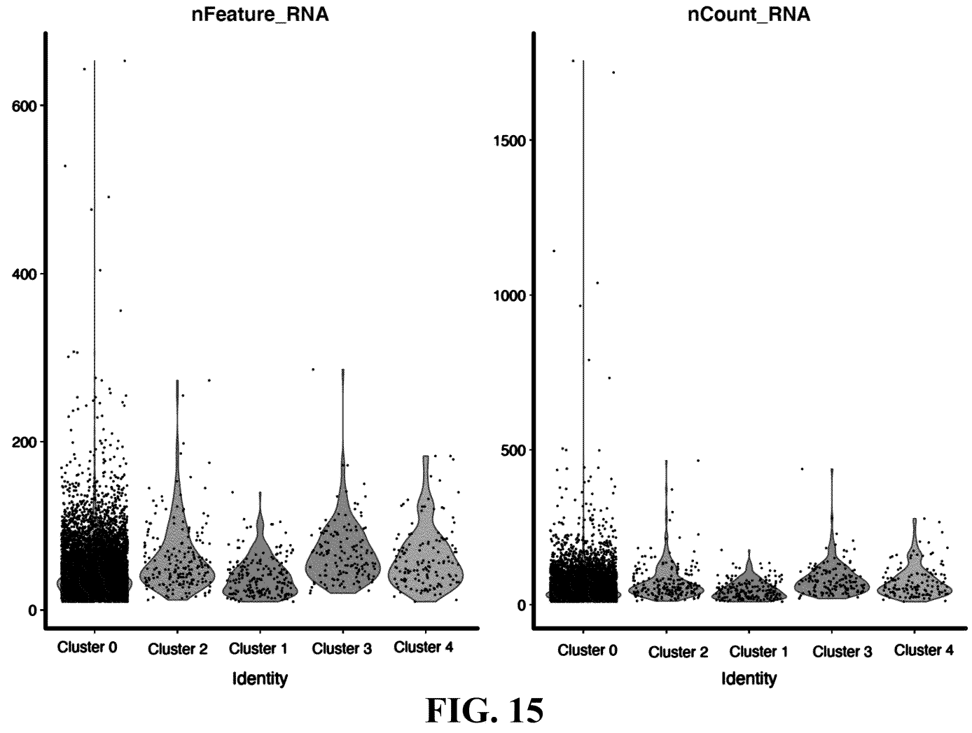

To determine if MGE-driven subpopulations are unique to MGH66, Applicants applied BacDrop to another K. pneumoniae clinical isolate (BIDMC35) (Table 2). BIDMC35 is a carbapenem-resistant isolate in which the carbapenem resistance resulted from a transposon disruption of the major porin gene ompK36 and the transposon-mediated high-level expression of a β-lactamase gene bla_{OXA-663}²³. Applicants again observed MGE-driven subpopulations, as in MGH66. Analyzing 9,748 BIDMC35 single cells that passed the analysis threshold at a sequencing depth of ~2000 reads per cell (FIGS. 3E & 3F, FIG. 15), Applicants identified three clusters driven separately by three different transposon genes, including an IS4321 family transposase (Cluster 2, 195 cells, 2%), the insH transposase (cluster 3, 146 cells, 1.5%), and an IS110 family transposase (cluster 4, 133 cells, 1.4%). Due to the presence of multiple copies of each of these transposase genes, Applicants could not resolve whether the high-level expression is caused by expression from a single copy or multiple copies of these transposase genes. Nonetheless, combining reads aligned to all copies of IS4321, insH, or IS110, the expression of each of these three transposase genes in their featured cluster is about 500 times higher than their expression in the rest of the population. Together with the observation in MGH66, it reinforces the finding that variable expression of MGEs may be one of the major drivers of population heterogeneity.

In BIDMC35, besides these MGE-driven subpopulations, Applicants observed another unique subpopulation (190 cells, 2%) driven by the 30- to 320-fold higher expression of a group of prophage genes (FIG. 3F, Table 3), compared to the rest of the populations, indicating that this cluster of cells was likely undergoing spontaneous phage induction. This observation is similar to the phage-induction subpopulation reported in the microSPLiT study⁹. Additionally, Applicants found the expression of bla_{OXA-663} is significantly lower in this phage-induction subpopulation (FIG. 3F), possibly caused by spontaneous phage induction.

### BacDrop Reveals Heterogeneous Stress Responses to Antibiotic Exposure

Finally, to determine if a perturbed population might have heterogenous transcriptional responses which are being masked by bulk-RNA-seq, Applicants analyzed the single cell responses to different antibiotic exposures from the BacDrop results. Under the conditions in which these samples were generated, the numbers of cells queried, and the level of resolution they were analyzed, Applicants did not detect obvious heterogeneity of response to ciprofloxacin or gentamicin. This is in contrast to what Applicants observed for meropenem treatment.

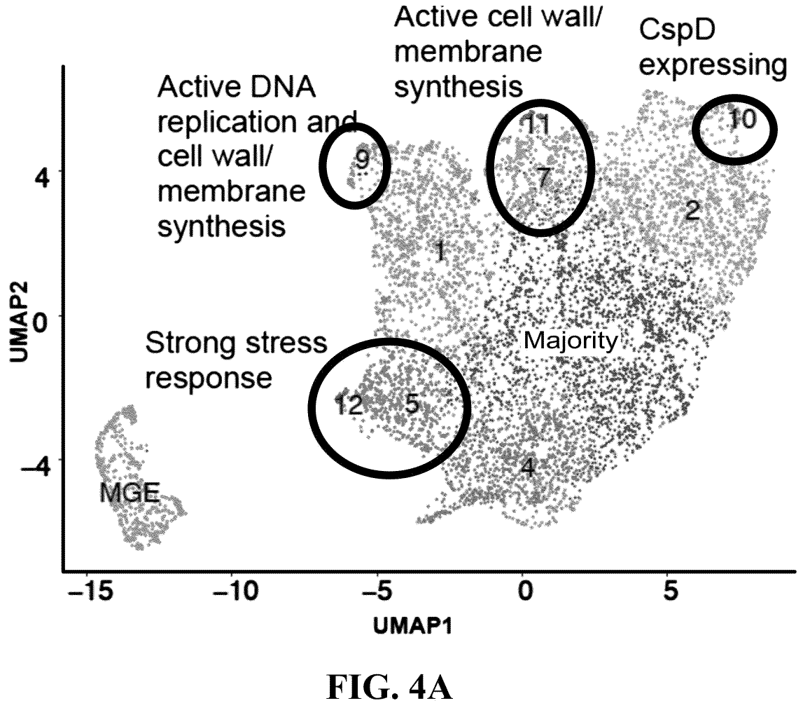

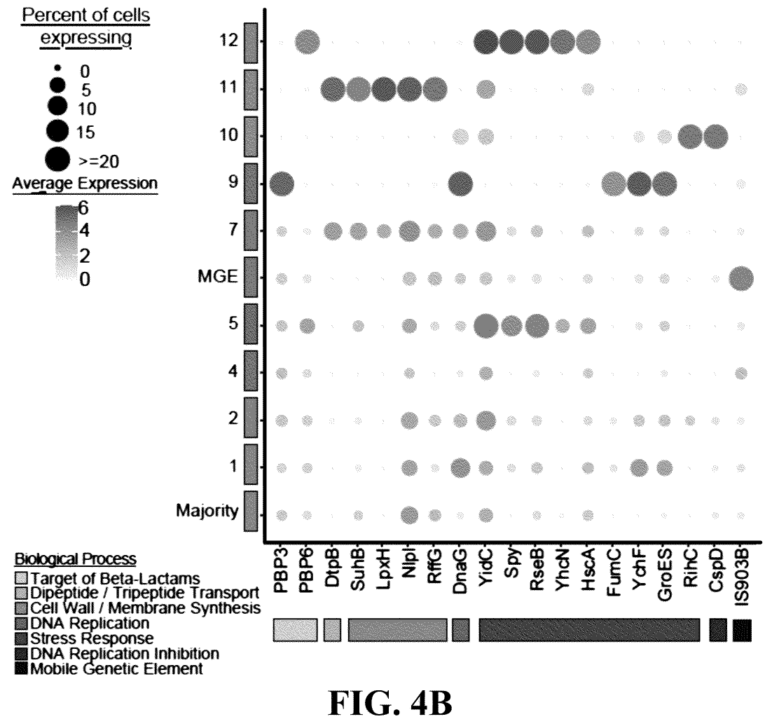

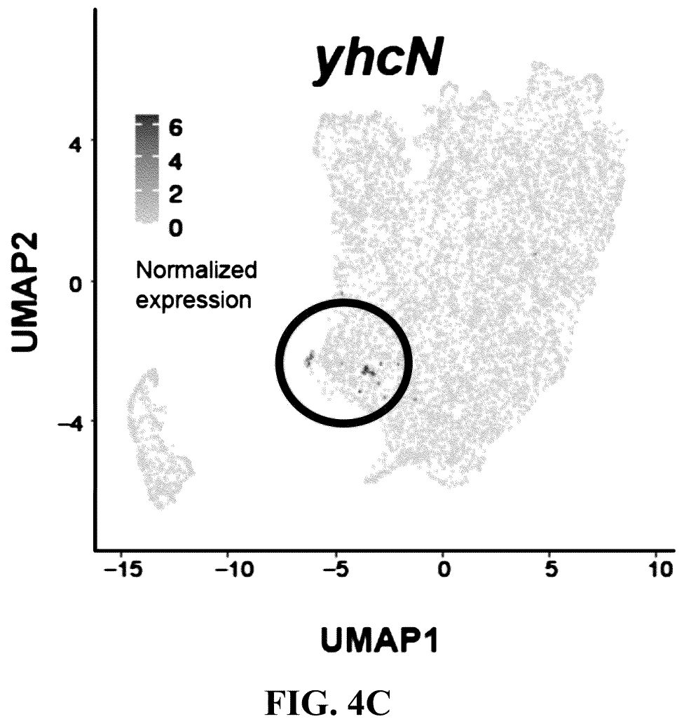

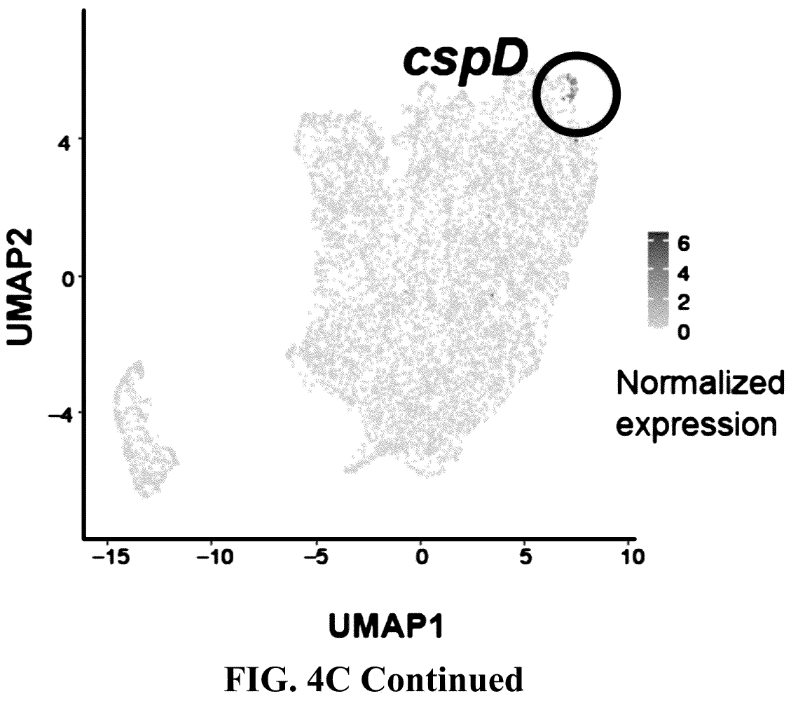

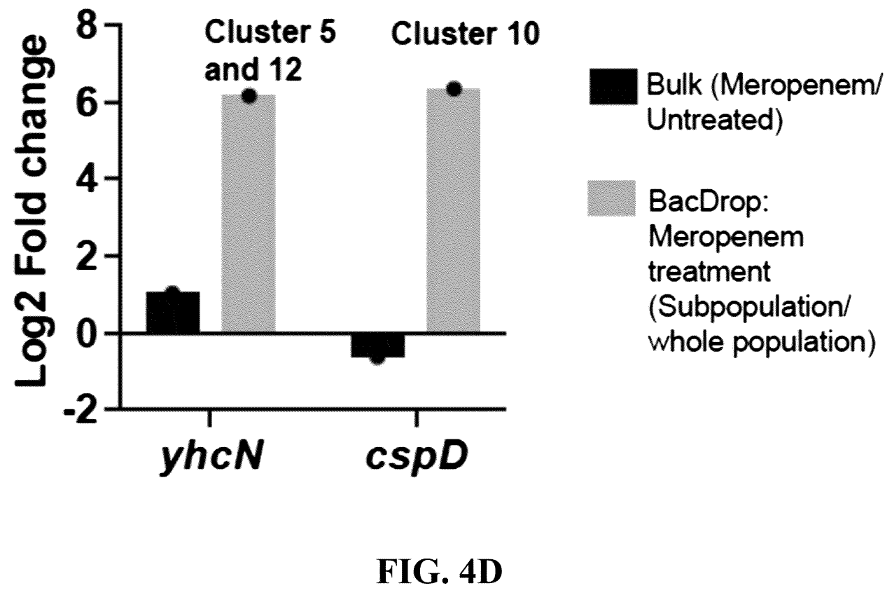

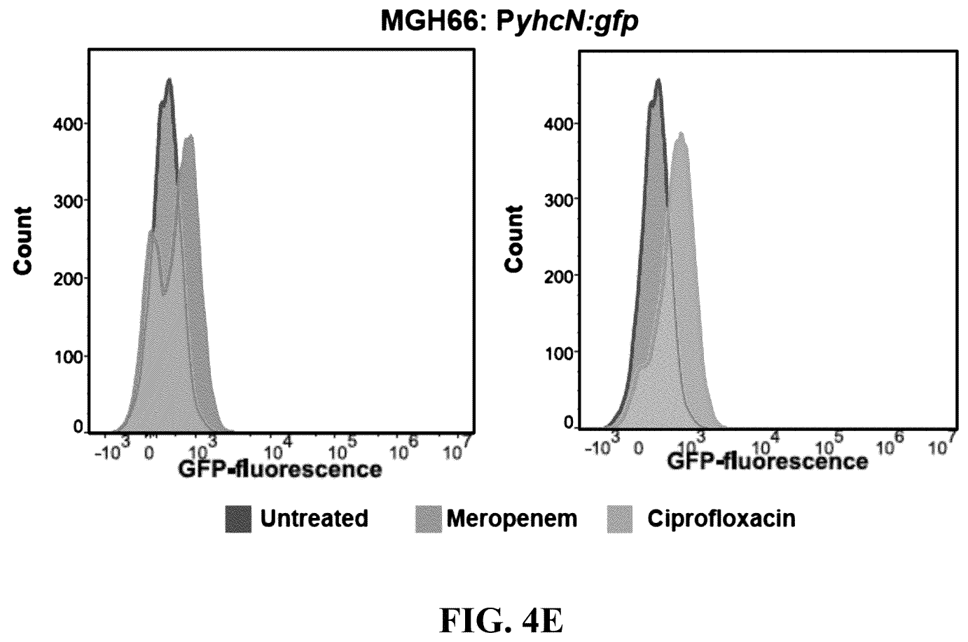

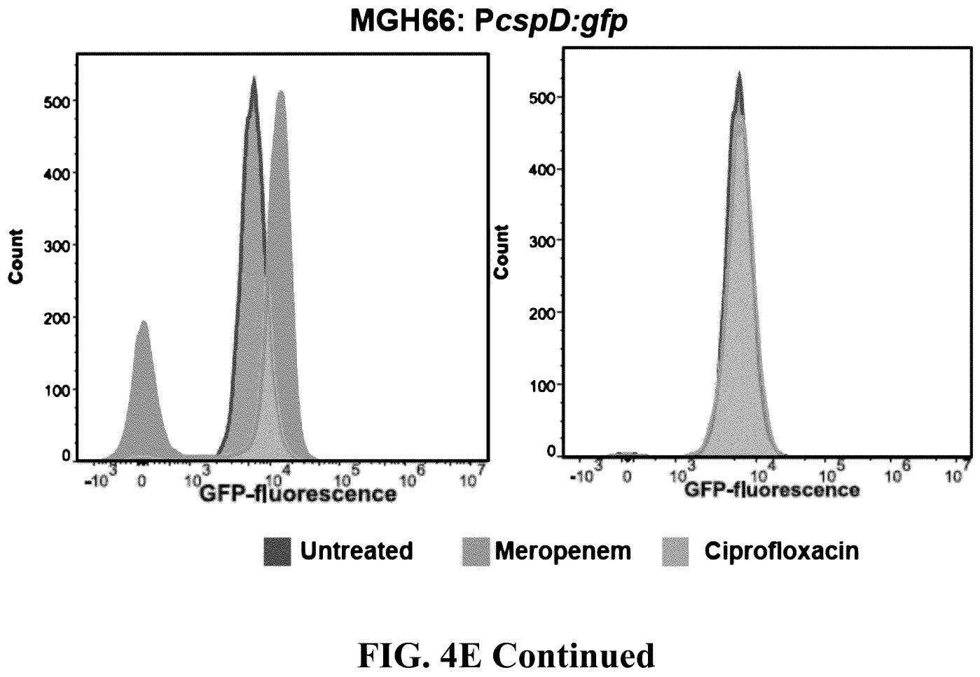

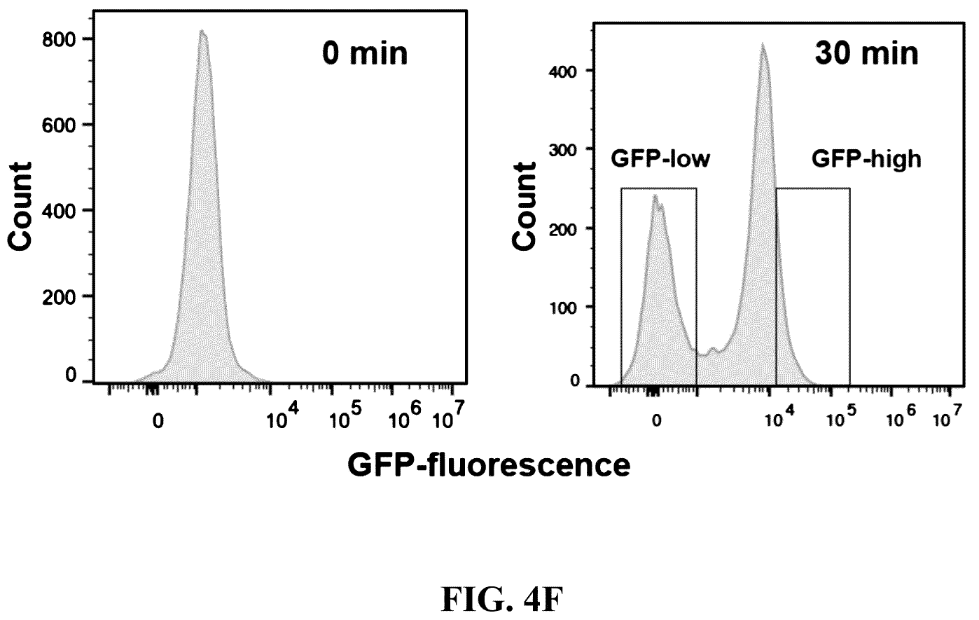

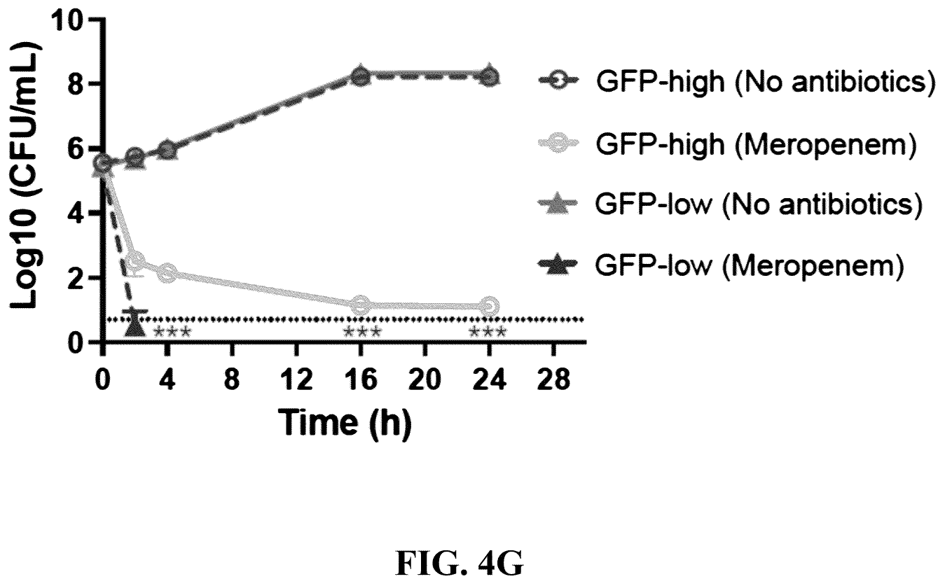

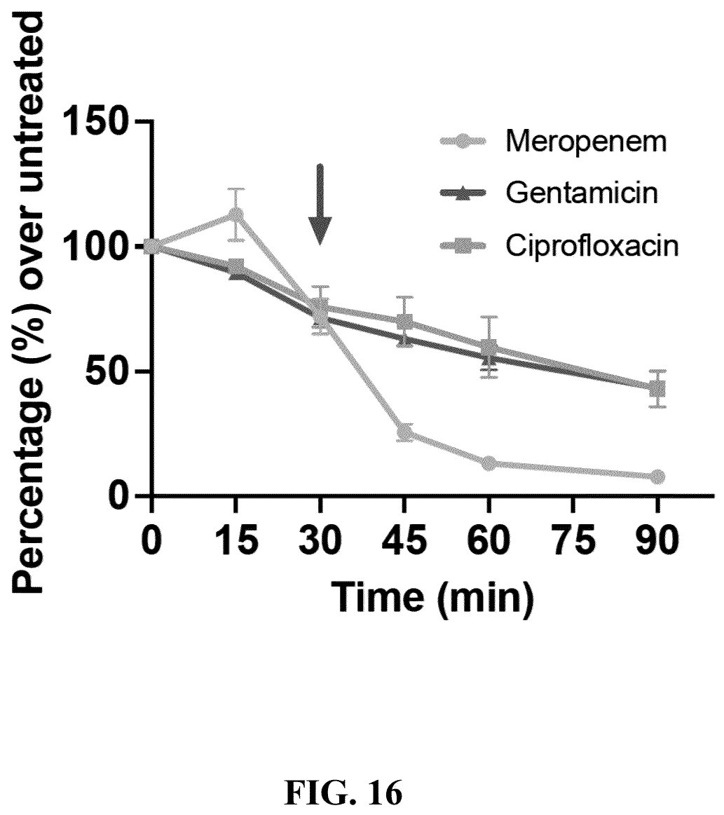

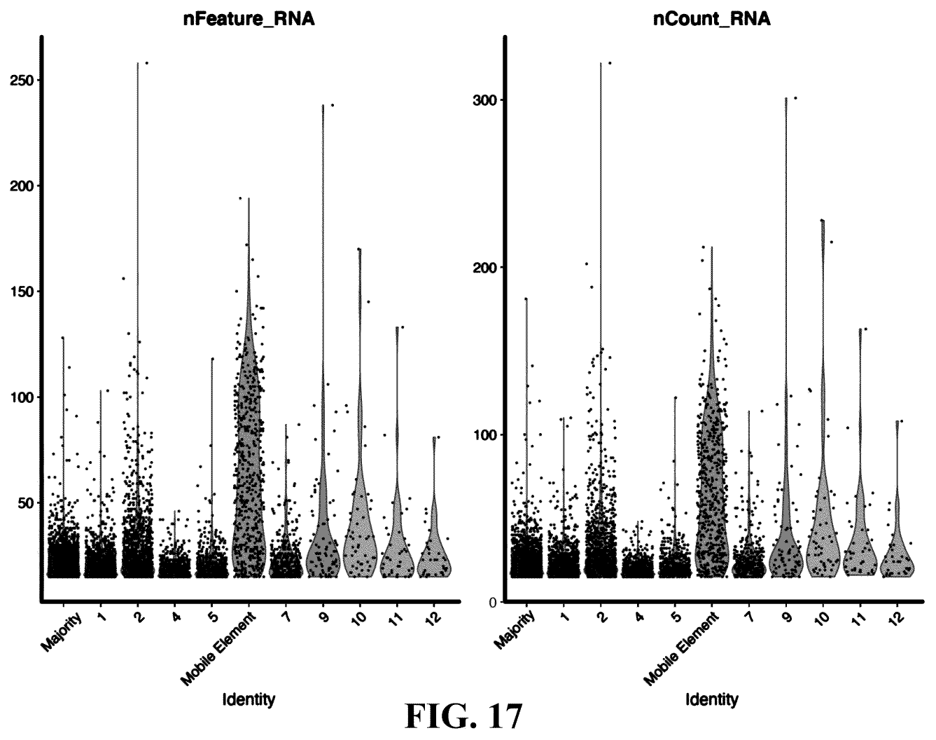

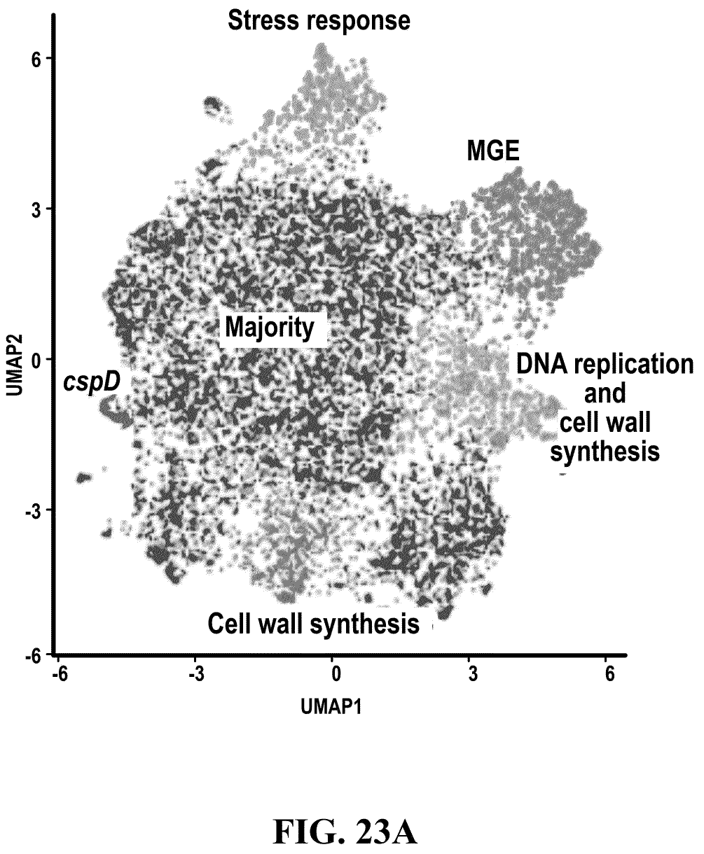

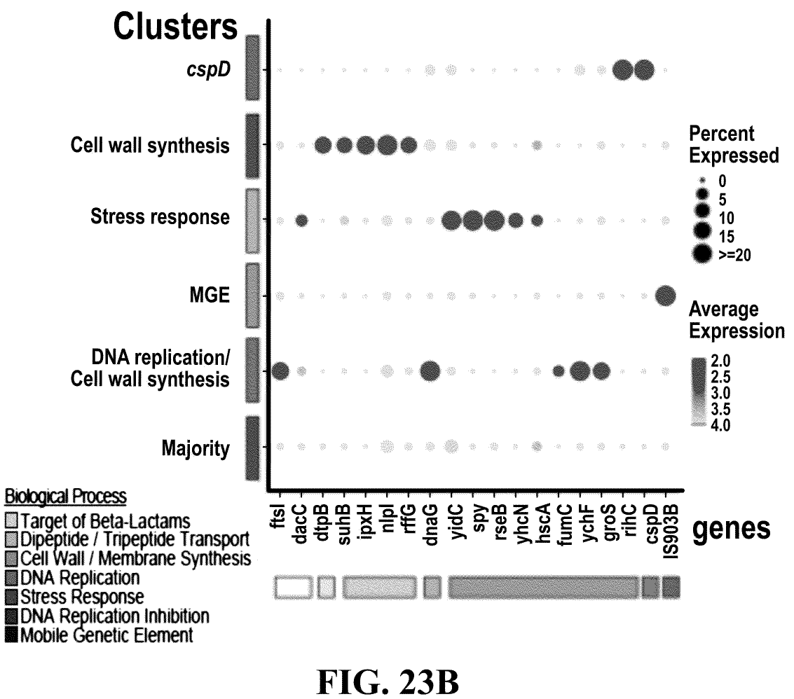

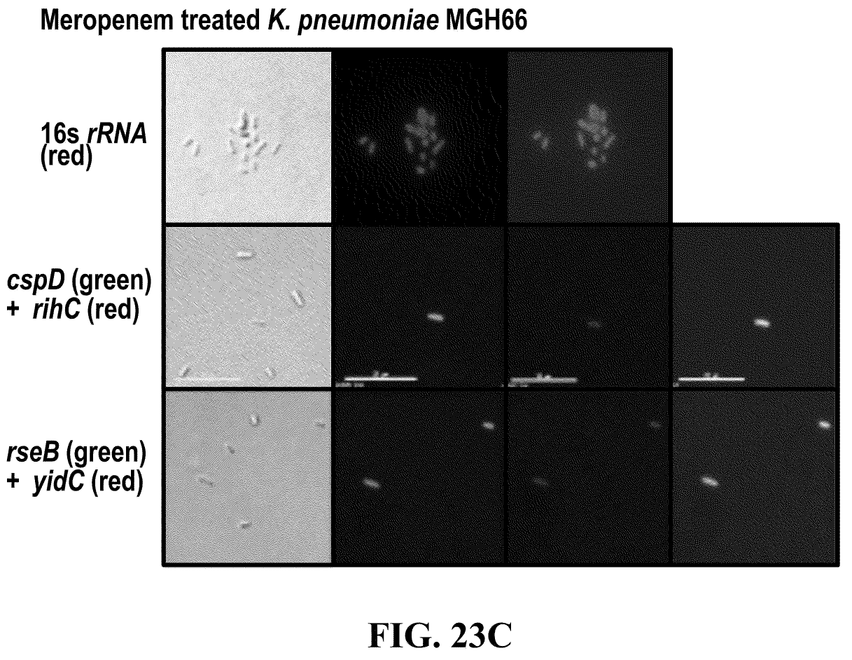

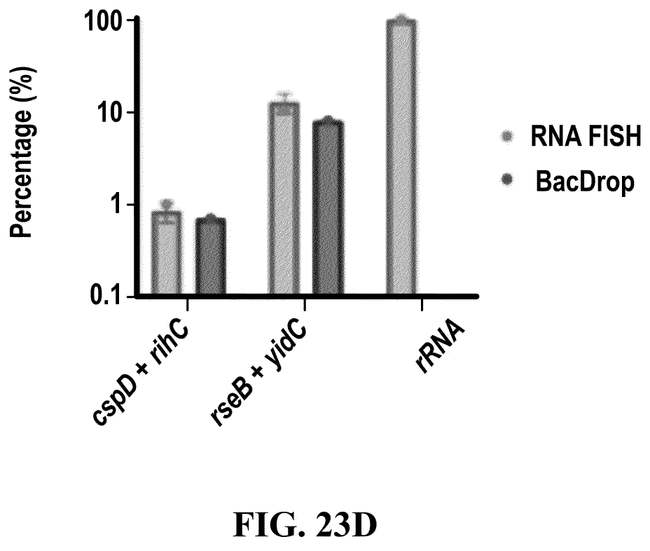

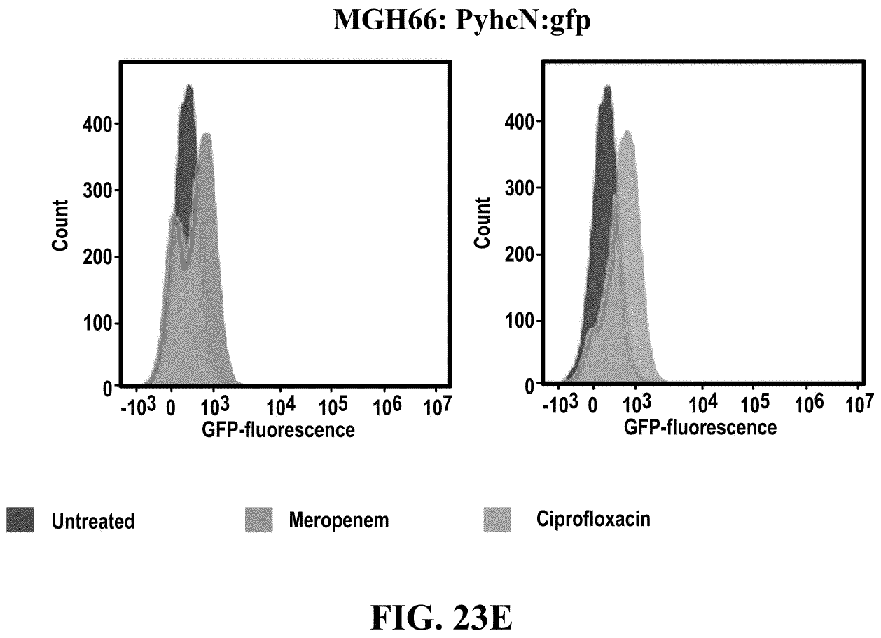

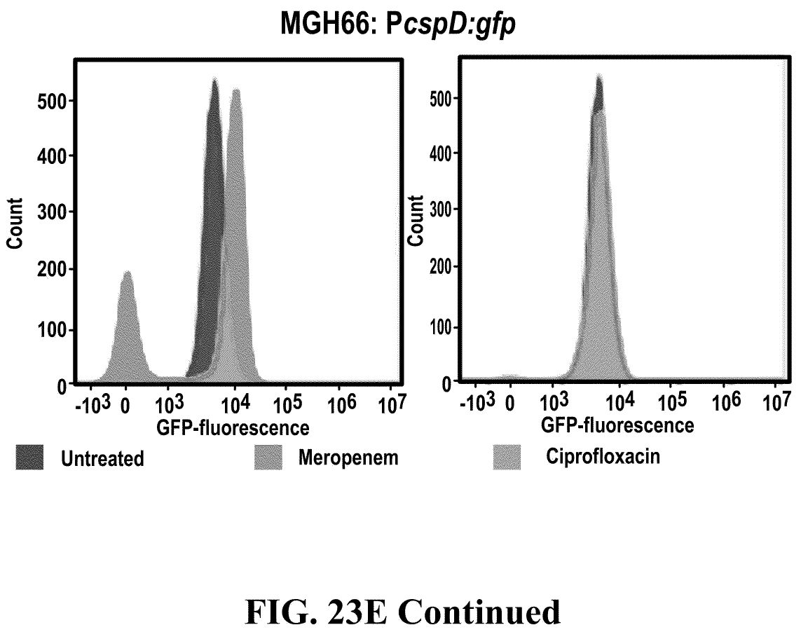

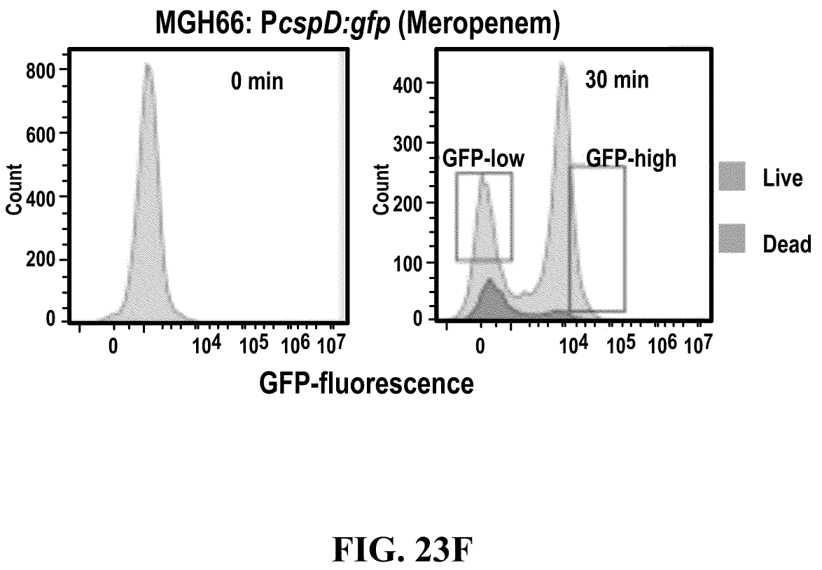

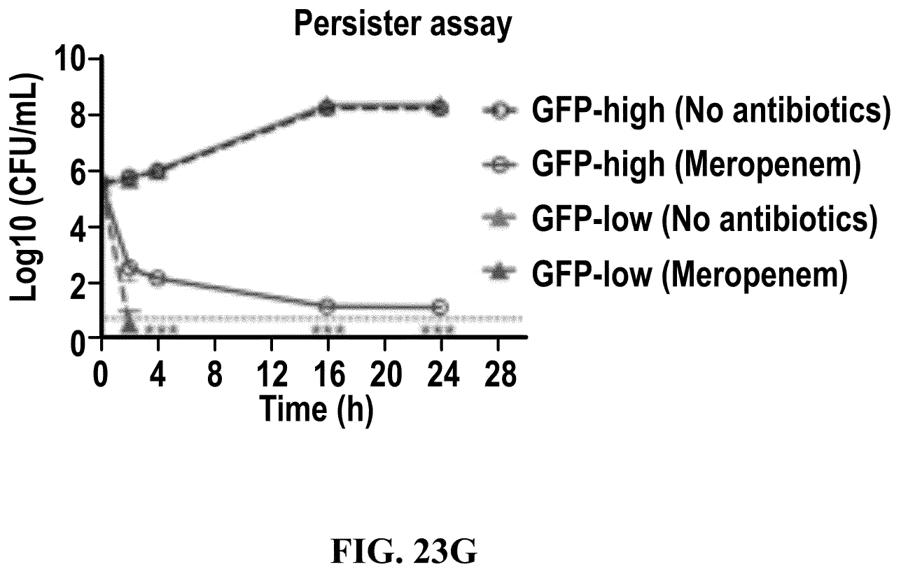

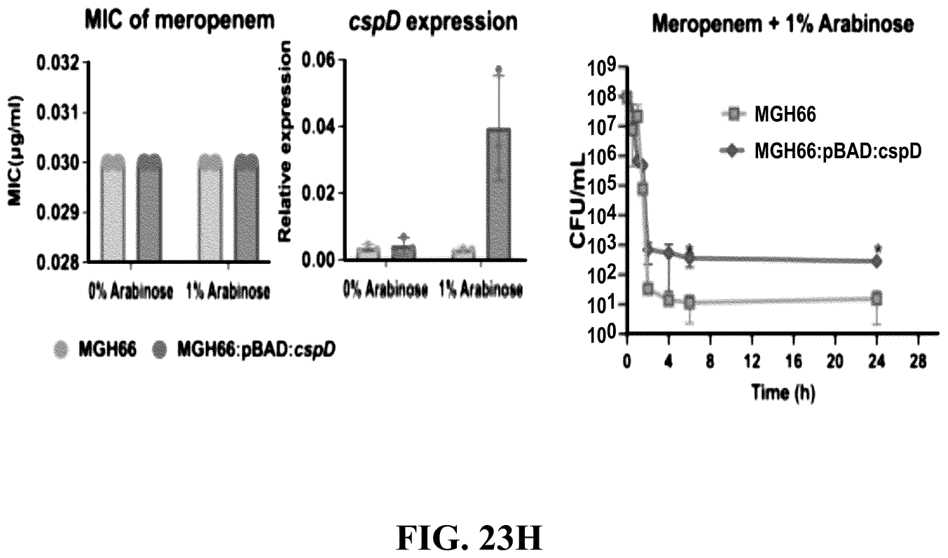

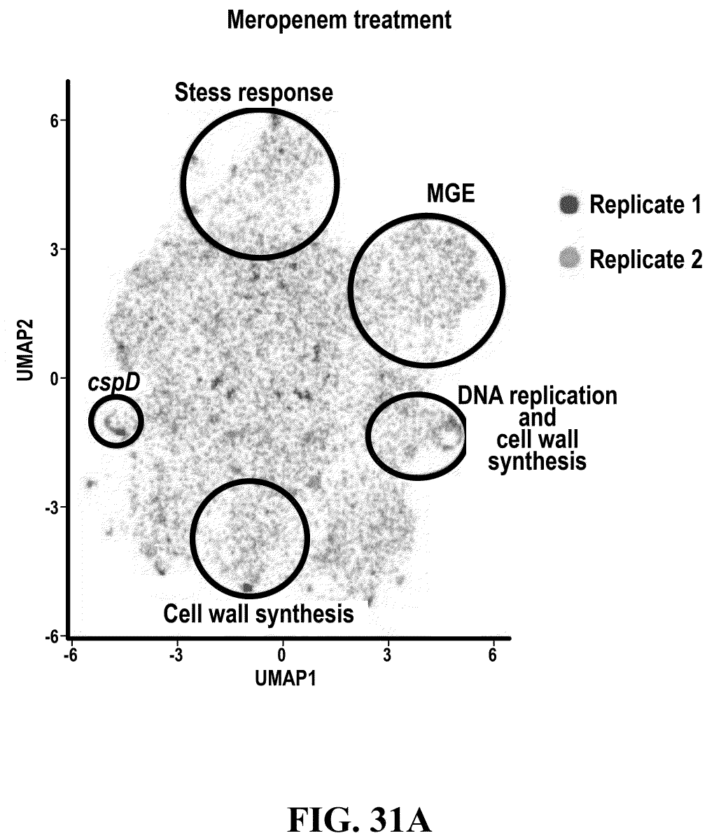

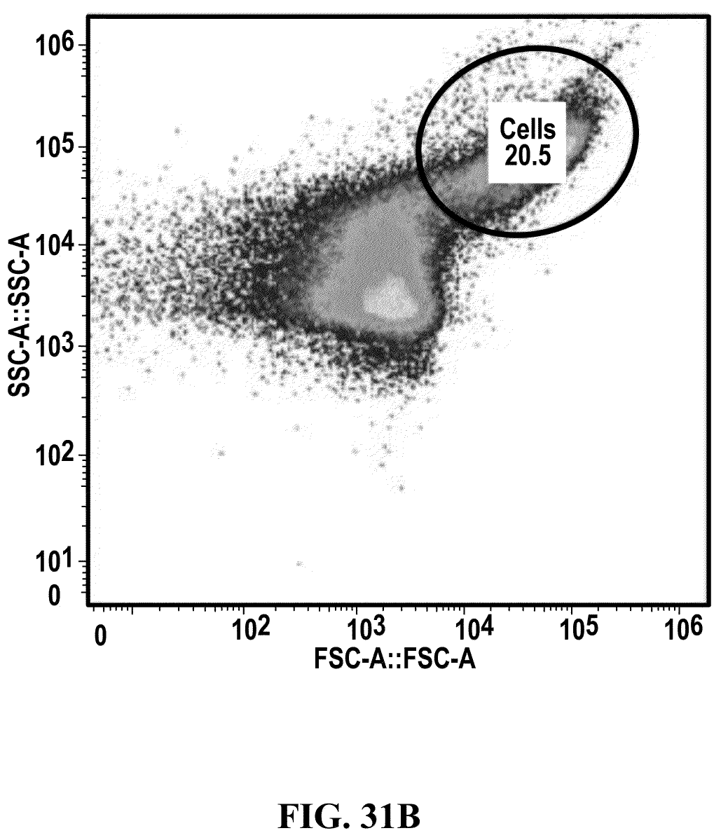

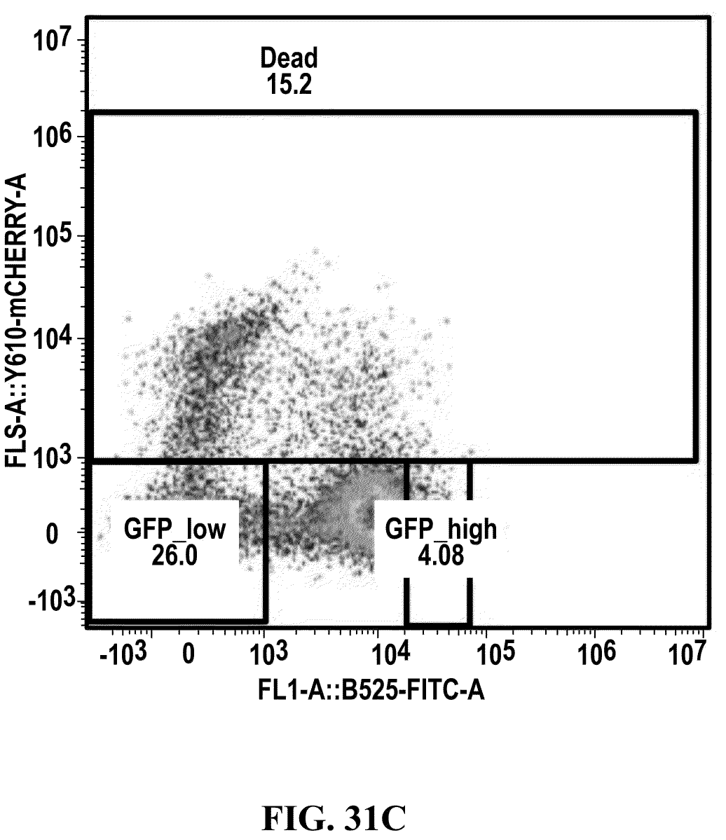

Applicants had been particularly puzzled by the fact that despite having the same impact on cell killing as ciprofloxacin and gentamicin at 30 minutes (FIG. 16), meropenem treatment only induced minimal transcriptional responses based on bulk RNA-seq analysis, in contrast to the other two antibiotics (FIG. 2B). Applicants thus analyzed the transcriptional response at the single cell level for the meropenem-treated samples and indeed, in addition to the MGE-driven subpopulation, found four interesting subpopulations with distinct molecular responses after meropenem treatment (FIG. 4A; FIGS. 23A and 23B; FIG. 31A). These four subpopulations include: i) a stress response cluster featuring high-level expression of stress response genes such as rseB, yidC, and yhcN; ii) a cell wall/membrane synthesis cluster featuring highly expressed genes involved in cell wall and membrane synthesis, i.e., nlpl and lpxH; iii) a cell wall synthesis and DNA replication cluster featuring highly expressed genes involved in DNA synthesis and cell wall/membrane synthesis such as dnaG and ftsI; and iv) a cspD-expressing cluster characterized by induced expression of cspD¹⁵, a DNA-binding toxin that inhibit DNA synthesis and induces the formation of persisters.

One subpopulation consisting of two clusters (clusters 5 and 12, 614 cells, 8%) showed the strongest stress response, compared to the rest of the population. While clusters 5 and 12 shared the same group of highly expressed genes, these genes were more highly expressed in cluster 12 compared to cluster 5. In this subpopulation, Applicants observed 5- to 200-fold higher expression of genes involved in cell envelope damage response (FIG. 4B, Table 4), including the sigma-E factor regulatory gene rseB, an ATP-independent periplasmic protein-refolding chaperone gene spy, and the membrane protein insertase gene yidC. In addition, Applicants also observed 10- to 100-fold higher expression of other stress response genes, including genes involved in a peroxide/acid stress response (yhcN) and cold-shock response (hscA). Interestingly, this subpopulation also showed 8- to 24-fold higher expression of dacC, the gene encoding penicillin-binding protein 6 (PBP6), a target of β-lactams such as meropenem.

In contrast, another subpopulation consisting of cluster 7 and 11 (432 cells, 5.7%) had high level expression of many genes involved in cell wall and membrane synthesis, with cluster 11 having higher expression of these genes compared to cluster 7 (FIG. 4B, Table 4). These genes include lpxH (lipid A biosynthesis), rffG (lipopolysaccharide (LPS) biosynthesis), secY and suhB (membrane protein transport and insertion), and nlpl (peptidoglycan biosynthesis and recycling). These results suggest that these cells may be actively synthesizing cell wall, cell membrane, and other cell surface structures. As β-lactam treatment has been previously reported to induce a futile cycle of cell wall synthesis to deplete peptidoglycan precursors and eventually lyse the cell²⁴, the induction of active cell wall/membrane synthesis observed in this subpopulation may represent the futile cycle of cell wall synthesis.

Another small, unique subpopulation consisting of cluster 9 (80 cells, 1%), features active DNA replication and cell division. Cells in this cluster showed 30- to 50-fold higher expression of genes essential for DNA replication and cell division (FIG. 4B, Table 4), including dnaG (encoding DNA primase) and ftsI (the essential cell division protein PBP3 and a target of 13-lactams). No strong response to cell wall/membrane damage was observed in this subpopulation. Instead, three other stress response factors are highly expressed, including groL, which encodes the heat-shock response chaperone GroEL, fumC, which encodes a backup enzyme in the TCA cycle when the FumA and FumB enzymes are damaged by reactive oxygen species, and ychF, which encodes a redox-regulated ATPase response to oxidative stress.

Finally, cluster 10 (53 cells, 0.7%) had high-level expression of cspD (FIG. 4B, Table 4), which encodes a toxin that inhibits DNA replication and induces the formation of persisters^{15,16}. In addition, rihC, the gene encoding ribonucleoside hydrolase RihC, an enzyme responsive to DNA damage, was also expressed exclusively in cluster 10. Previous studies have shown that the induction of rihC is independent of LexA²⁵, the key transcriptional regulator of the SOS response to DNA damage. Consistent with previous reports, no genes in the SOS response pathway were upregulated in cluster 10, suggesting that cells in cluster 10 may be responding to DNA damage in an SOS-independent way, which might facilitate the formation of persisters (FIG. 4B).

Having identified genes highly expressed in specific subpopulations after meropenem treatment (FIG. 4B, Table 4), Applicants used RNA fluorescence in situ hybridization (FISH) to validate the finding of the “stress response” and “cspD-expressing” subpopulations. Applicants simultaneously probed for two genes highly co-expressed in the “stress response” cluster, rseB and yidC, and confirmed the existence of this cluster (FIGS. 23B and 23C). Similarly, Applicants simultaneously probed for cspD and rihC, two genes highly co-expressed in the “cspD-expressing” cluster to confirm their existence (FIGS. 23B and 23C). The percentage of cells making up each cluster as detected by RNA-FISH and BacDrop were very similar (FIG. 23D).

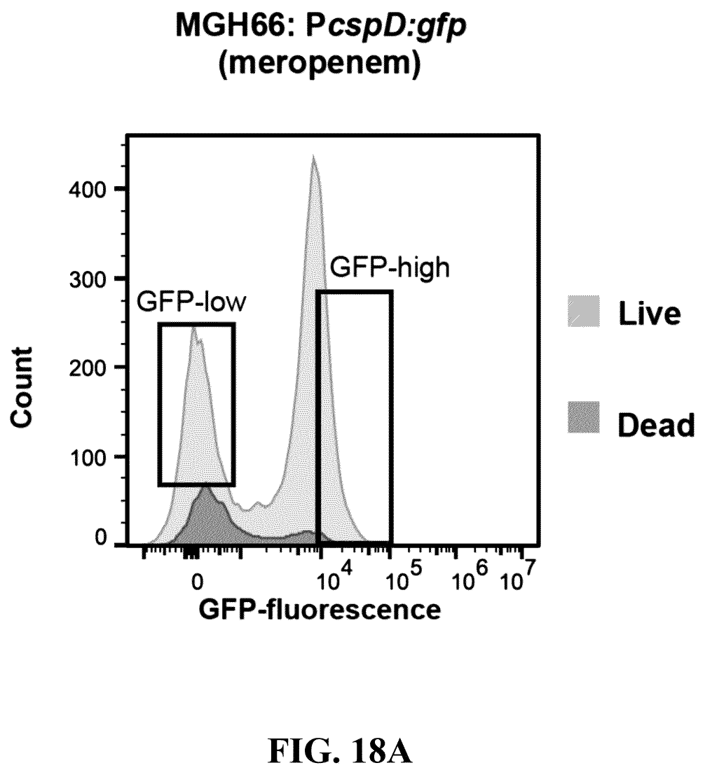

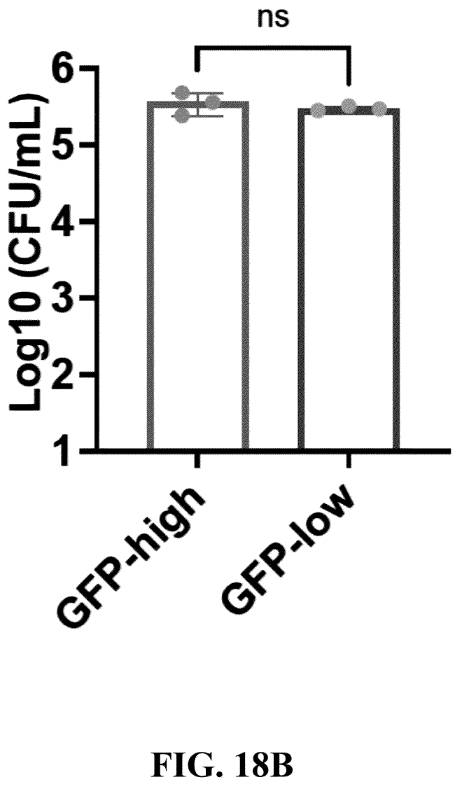

Applicants also used fluorescence cytometry to validate the expression patterns of genes in the “stress response” and “cspD-expressing” clusters. Applicants engineered MGH66 reporter strains expressing the short-lived green fluorescent protein²⁶ driven by the promoter of yhcN, a “stress response” gene (MGH66:PyhcN:gfp), or cspD (MGH66:PcspD:gfp) (FIGS. 4C, 4D and 4E). Indeed, meropenem, but not ciprofloxacin, induced heterogeneous expression of both yhcN or cspD, consistent with BacDrop results (FIG. 23E). Applicants confirmed that the cells with reduced fluorescence of MGH66:PcspD:gfp (GFP-low) were live and not simply dying using a live-dead stain and plating for colony forming units (FIG. 18; FIGS. 31B and 31C). Interestingly, the GFP-low population actually had lower fluorescence than the untreated population, suggesting that in fact, there may be suppression of cspD expression in this population. Of note, the fractions of cells highly expressing these two genes were greater by flow cytometry than by BacDrop; this would be consistent with the fact that RNA and protein levels are not necessarily well correlated in single cells⁶, as one copy of mRNA can produce one to hundreds of copies of proteins and mRNA is less stable than protein. Nevertheless, the heterogeneous responses were observed by both methods of measurement.

In summary, under meropenem perturbation, Applicants observed strong heterogeneous responses driven by various stress response pathways that had been previously masked by bulk RNA-seq results. Rather than uniformly turning on a specific stress response pathway in all cells, a diverse range of stress responses appear to be induced in different subpopulations, which could potentially contribute to heterogeneous cell fates such as cell lysis or antibiotic tolerance.

### BacDrop Identifies a Subpopulation With Reduced Meropenem Efficacy and Increased Persisters

Given CspD’s reported role in inducing the formation of antibiotic-tolerant persisters in E.coli^{15,16}, Applicants asked whether antibiotic tolerant cells might make up a greater percentage of the GFP-high than the GFP-low subpopulation, with cells in which cspD had been induced surviving preferentially under meropenem treatment. Because CspD plays a role in persister formation rather than maintenance, Applicants anticipated that some cells might induce cspD and thus make high levels of GFP, become antibiotic tolerant, then turn off CspD expression resulting in reduced expression of GFP as the GFP is degraded. Despite this possibility, Applicants nevertheless hypothesized that the GFP-high subpopulation might still be enriched for tolerant cells compared to the GFP-low subpopulation. Applicants performed fluorescence activated cell sorting (FACS) of the MGH66 GFP transcriptional reporter strain (MGH66:PcspD:gfp) coupled with dead-cell stain, after a 30-minute exposure to meropenem. Using flow cytometry coupled with dead-cell stain, after a 30-minute exposure to meropenem, Applicants sorted live cells into the two subpopulations of MGH66: PcspD:gfp (GFP-high and GFP-low) (FIG. 4F; FIG. 23F) directly into liquid media with and without meropenem. Applicants then plated cells from the ensuing 4 samples onto solid agar without antibiotics to enumerate surviving bacteria over time (FIG. 4G; FIG. 23G). Indeed, the GFP-high subpopulation was enriched for meropenem tolerant cells compared to the GFP-low subpopulation, with evidence of a persister population in the GFP-high but not the GFP-low subpopulations (FIG. 4G; FIG. 23G). Consistent with this observation, Applicants also observed ~ 100 times more persister cells in a MGH66 strain over- expressing cspD, though the over-expression of cspD did not reduce antibiotic susceptibility (FIG. 23H). Together, these results confirmed the role that cspD plays in persister formation in a subpopulation of K.penumoniae MGH66.

### Discussion

Applicants report a novel bacterial scRNA-seq method, BacDrop, that is robust and reproducible, and leverages droplet-based technology to enable the study of the transcriptional program of both large and smaller numbers of single bacterial cells. Applicants applied BacDrop to study the naturally occurring heterogeneity in a heretofore presumed uniform culture of bacteria and its heterogenous responses to perturbation. Previous genome-wide single cell studies on smaller numbers of cells (hundreds to thousands) have to date focused predominantly on samples with artificially-created heterogeneity, created as mixes of different populations, for method demonstration of between population heterogeneity⁷⁻¹⁰. Here, Applicants characterized both a stable and a dynamic population of cells derived from the same bacterial isolate, and demonstrated a diversity of states within the stable population and the heterogenous responses in the dynamic population after perturbation with antibiotic. Applicants find that in both cases, the ability to analyze large numbers of cells (thousands to tens of thousands, with the potential to go much higher) enables higher resolution identification of small subpopulations.

BacDrop revealed within population heterogeneity driven predominantly by the expression of MGEs in the presumed uniform cultures of K.pneumoniae. While MGEs drive genetic diversity by their relatively random movement in the genome, Applicants find that the expression levels of individual MGEs are also highly variable within populations of MGH66 and BIDMC35 (FIG. 3 and FIG. 22). Whether this heterogeneity in expression is due to genotypic variation resulting from the movement of genetic elements such as transposon insertions or phase variation, to transcriptional changes in response to stochastic/or local microenvironmental cues, or to epigenetic mechanisms, remains to be understood. However, the consequences of such variation can be significant. Applicants propose that the elevated transposition frequencies found only in a subpopulation of MGH66 likely contribute to the strain’s propensity to become carbapenem resistant, given Applicants’ previous demonstration of the role that high-level transposon mutagenesis plays in carbapenem resistance²². Applicants also identified a very small population of cells (0.25%) in a distinct metabolic state, with signatures of stringent response, nutrient starvation and scavenging, biofilm formation and cell filamentation. The high up-regulation of ispH, a gene involved in antibiotic tolerance and stringent response²⁷, suggests that this group of cells might be undergoing metabolic stress, perhaps maybe even with a potential role in antibiotic tolerance phenotypes.

Meanwhile, in response to a perturbation such as exposure to the important antibiotic meropenem, Applicants observed a wide range of transcriptional responses that are otherwise masked by bulk RNA-seq. Interestingly, a diverse range of stress responses appear to be induced in different subpopulations rather than a single or uniform response occurring in all cells; this subpopulation diversity could potentially contribute to heterogeneous cell fates or phenotypic outcomes, including cell lysis or antibiotic tolerance. It remains to be understood whether this heterogeneous stress response occurring in subpopulations is a general phenomenon in response to stressors or it is specific to certain types of stresses such as meropenem, which may be working in a much more pleiotropic manner than is assumed, thereby eliciting a wide range of stress responses. Meanwhile, Applicants examined one such subpopulation defined by high-level expression of cspD, a gene encoding a toxin that inhibits DNA replication and induces the formation of persisters, but that is not significantly upregulated in bulk RNA-seq results. Using both FISH and flow cytometry, Applicants confirmed that cspD is indeed induced in a subpopulation after meropenem treatment. Moreover, Applicants found a higher survival rate under meropenem treatment in the cspD-induced subpopulation, pointing to its role in antibiotic tolerance within this subpopulation and more generally, to the importance of certain responses in some subpopulations in surviving the lethal effects of antibiotics. Compared to other subpopulations, this cspD-induced subpopulation also showed unique SOS-independent DNA damage response, which could be potentially linked to the induction of cspD.

Applicants have shown that BacDrop can be applied to studies of both large and smaller numbers of cells, depending on the biological question of interest. Importantly, Applicants demonstrated that studying large numbers of cells can more robustly identify population heterogeneity including rare subpopulations even with fewer mRNA genes per cell (FIG. 30). With the ability to characterize large numbers (millions) of cells using BacDrop without requiring any prior knowledge of the genomes of interest, the limitation for understanding the single cell transcriptional programs within complex communities is no longer technical. Instead, one of the major limitations becomes that of sequencing cost, which currently necessitates a compromise between sequencing large numbers of cells and the sequencing depth of individual cells. There is value both in large numbers of cells sequenced less deeply, as well as deep sequencing of lower numbers of individual cells to obtain information on more unique genes. With the rapid development of sequencing technologies and platforms, one can easily imagine that such compromises may no longer be required in the near future.

New computational methods for analysis are also needed. In this study, Applicants analyzed BacDrop data using programs established for mammalian scRNA-seq. More efficient computational pipelines that easily accommodate these much larger cell numbers, and, importantly, are tailored towards bacterial genomes and their genetic architecture will greatly increase the power of BacDrop. This will require parallel efforts to improve annotation of bacterial genomes and to better define operon structure and networks in many different bacterial species, beyond E.coli.

Applicants have developed BacDrop to be a versatile, high-throughput method to characterize the transcriptional programs of bacterial cells in the range from thousands to millions. By doing so, one can reveal both heterogeneity in cell types (species) in a mixed community or cell states in a single strain under stable or dynamic conditions. To demonstrate its utility, Applicants validated BacDrop in four different pathogenic bacterial species. Applicants expect that BacDrop could be easily adapted to more species, including commensal bacterial strains and other environmental species. Applicants thus propose that BacDrop will become a powerful tool for a broad range of studies, including studies focused on elucidating phenotypic heterogeneity, understanding bacterial interactions in microbial communities, dissecting host-pathogen interactions, expanding Applicants’ knowledge of the microbiome beyond genomes and bulk metabolomics, and investigating the emergence of antibiotic resistance, persistence and tolerance.

### Materials and Methods

### Experimental Methods

### Bacterial Strains and Culture

Bacterial strains used in this study are listed in Table 2. K.pneumoniae, E.coli, and P. aeruginosa were cultured in Luria-Bertani (LB) medium or Mueller-Hinton Broth (MHB) with shaking at 37° C.E.faecium was cultured in Todd-Hewitt broth (THB) with shaking at 37° C. For the GFP experiment, E.coli strains expressing gfp driven by different promoters were cultured separately in LB at 37° C. Early exponential growth phase cells (OD₆₀₀ ~0.2) were collected and fixed immediately. For the antibiotic treatment experiment, K.pneumoniae clinical isolate MGH66 was cultured in MHB at 37° C. until early exponential phase. Then the culture was diluted to OD₆₀₀ 0.05 in MHB. After growing for two doublings (~40 min, OD₆₀₀ ~0.2), the cultures were split into four equal volume cultures. One culture was left untreated, while the other three were treated with relevant antibiotic at breakpoint concentrations set by the Clinical and Laboratory Standards Institute (CLSI): 2 µg/mL for meropenem, 1 µg/mL for ciprofloxacin and 4 µg/mL for gentamicin. After 30 min, 7 mL of cells were collected from each of these cultures, and immediately proceeded with the cell fixation protocol. For the bulk RNA-seq experiments, samples were collected using the same treatment schemes, and three biological replicates were included in each condition. For the BIDMC35 experiment, BIDMC35 was cultured in LB medium at 37° C. until early exponential phase (OD₆₀₀ ~0.2). 7 mL cells were then collected and immediately proceed with the cell fixation protocol.

### Cell Fixation and Permeabilization

All reagents (Table 5) for cell fixation and permeabilization were kept ice-cold. Up to 10 billion bacterial cells grown at specified conditions were collected by centrifuging at 5525 × g for 10 min at 4° C. The supernatant was removed and cell pellets were resuspended in 7 mL fresh, ice-cold 4% formaldehyde (Sigma, CN 47608) in 1x PBS and incubated with shaking overnight at 4° C. (Digital Platform Rocker Shaker, VWR). Following overnight fixation, cells were centrifuged at 5525 × g for 10 min at 4° C. The supernatant was removed and cells were resuspended in 7 mL PBS-RI (1x PBS supplemented with 0.1 U/µL NxGen RNase inhibitor (Lucigen, CN 30281)). Cells were centrifuged again at 5525 × g for 10 min at 4° C. and pellets were resuspended in 700 µL PBS-RI. Subsequent centrifugations were carried out at 7000 × g for 5 minutes at 4° C. Applicants noticed that some species, e.g., P.aeruginosa, do not pellet very well at this centrifugation speed (FIG. 25B). Thus, Applicants recommend optimizing the centrifugation speed for the specific species of interest. Cells were centrifuged and resuspended in 700 µL50% Ethanol in PBS-RI. Cells were then washed twice with PBS-RI. After the second wash, cells were resuspended in 1 mL100 mM Tris-HCL (pH 7.5) supplemented with 0.1 U/uL NxGen RNase inhibitor. Cells were diluted 100x and quantified using a hemocytometer (VWR, CN 102966).

Applicants found that the number of cells in a permeabilization reaction is critical for achieving sufficient permeabilization. Insufficient permeabilization may result in inefficient rRNA depletion and gDNA removal. For 1 permeabilization reaction, up to 40 million cells were centrifuged and resuspended in 250 µL0.04% Tween-20 in 1x PBS. If more cells are desired, multiple parallel reactions can be set up. Immediately following a 3-minute incubation on ice, 1 mL cold PBS-RI was added, and cells were spun down and resuspended in 200 µL lysozyme mix (100 mM Tris (pH 8.0), 50 mM EDTA pH 8.0, 0.25 U/µL NxGen RNase Inhibitor, 2.5 mg/mL lysozyme). Cells were then incubated at 37° C. for 15 minutes. After the incubation, 1 mL PBS-RI was added and cells were washed twice with 175 µL PBS-RI. After the second wash, cells were resuspended in 150 µL PBS (without RNase inhibitor added) and cell concentrations were measured.

### In-cell rRNA Depletion and gDNA Removal

Immediately after cell permeabilization, up to 40 million cells were centrifuged and resuspended in 11 µL nuclease-free H₂O. 2 µL NEBNext Bacterial rRNA depletion solution and 2 µL Probe Hybridization Buffer (NEB, CN E7850) were mixed with cells on ice. The hybridization was conducted per the following (lid temperature set to 55° C.): 50° C. for 2 minutes, ramp down to 22° C. at 0.1° C. /second, and hold at 22° C. for 5 minutes. Probe hybridization was immediately followed by RNase H digestion by mixing the probe-hybridized cells with 2 µL RNase H reaction buffer, 2 µL Thermostable RNase H (NEB, CN E7850), and 1 µL nuclease-free H₂O, followed by a 30-minute incubation at 50° C. (lid temperature set to 55° C.). The 20 µL reaction was centrifuged and resuspended in 10 µL DNase-RI buffer (1 µL DNase I reaction buffer (Sigma, CN AMPD1), 1 µL DNase I (Sigma, CN AMPD1), 0.025 µL NxGen RNase inhibitor (Lucigen, CN 30281), 8 µL nuclease-free H₂O). The reaction was incubated at room temperature for 30 minutes, and the DNase treatment was stopped by adding 1 µL Stop Solution (50 mM EDTA) and incubating at 50° C. for 10 minutes. After the incubation, cells were centrifuged and washed twice with 100 µL PBS-RI. After the second wash, cells were resuspended in 20 µL0.5x PBS supplemented with 1 U/µL SUPERase-In RNase inhibitor and used immediately for in-cell reverse transcription.

### In-cell Reverse Transcription, Round 1 Cell Barcoding and Sample Multiplexing

The round 1 plate barcoding and sample multiplexing is achieved via RT reactions in 384- or 96-well plates. 384 RT primers (Table 1) containing UMI sequences and round 1 plate barcodes (CB1) were synthesized at Integrated DNA Technologies at 100 µM concentration. The primers were diluted with ddH₂O to a working concentration of 25 µM, and 2.5 µL of each primer was aliquoted into individual wells of the 384- or 96- well plates. The rRNA and gDNA depleted cells was diluted with nuclease-free H₂O supplemented with 1 U/µL SUPERase-In RNase inhibitor and added to the plate containing RT primers (1 µL cells per well). Then the cell-primer mix was incubated at 55° C. for 5 minute and immediately put on ice. For each well, the RT master mix, containing 0.25 µL DTT (100 mM), 0.25 µL dNTP (10 mM each), 0.25 µL SUPERase-In, 1 µL RT buffer, 0.25 µL Maxima H Minus reverse transcriptase (Thermo Scientific, CN EP0753), was added and the RT reaction was incubated as follows (set lid temperature to 60° C.): 50° C. for 10 min, 3 cycles of [8° C. for 12 s, 15° C. for 45 s, 20° C. for 45 s, 30° C. for 30 s, 42° C. for 2 min, 50° C. for 3 min], 50° C. for 5 min, hold at 4° C.

### In-cell cDNA 3′ poly-A Tailing

After RT, cells were recovered and pooled from 384- or 96- well plate. For multiplexed samples, Applicants prefer to only pool cells from the same sample together, generating individual pools for each sample. This will allow more flexibility to adjust cell numbers of different samples during droplet generation. Then the pooled cells were centrifuged and each pool was resuspended 40 µL nuclease-free H₂O supplemented with 1 U/µL SUPERase-In. The poly-A tailing reaction was set up as the following: 38 µL cells, 5 µL 10x terminal transferase buffer, 1 µL dATP (100 mM) (NEB, N0446S), 1 µL SUPERase-In, 5 µL terminal transferase (TdT) (NEB, CN M0315L). The reaction was incubated at 37° C. for 1 hour. Then 10 µL0.2 M EDTA was added to each 50 µL reaction and incubated at room temperature for 10 minutes. Cells were then centrifuged and resuspended in 10 µL nuclease-free H₂O supplemented with 1 U/µL SUPERase-In. Cells were diluted and concentrations were quantified.

### Droplet Generation

The Chromium Next GEM Chip H (10x Genomics, PN 1000161) and Chromium Next GEM Single Cell ATAC Library & Gel Bead Kit (10x Genomics, PN 1000176) was used for the droplet generation. The unused wells were filled with 70 µL (row 1), 50 µL (row 2), and 40 µL (row 3) 50% glycerol solution. The desired number of cells was diluted to 33.75 µL with nuclease-free H₂O supplemented with 1 U/µL SUPERase-In. Right before loading the chip, 33.75 µL cells were mixed with a PCR master mix containing 37.5 µL KAPA HiFi HotStart ReadyMix (Roche, CN KK2602), 2.25 µL second strand synthesis primer SMRT_dT (10 µM) (Table 6), and 1.5 µL Reducing Agent B (10x Genomics, PN 2000087). The chip was loaded with 70 µL PCR-cell mix (row 1), 50 µL Gel Beads (row 2), and 40 µL partitioning oil (row 3), and run on the Chromium system. Approximately 100 µL droplet emulsion was obtained in row 3.

### Second Strand cDNA Synthesis and Round 2 Droplet Barcoding in Droplets

To increase thermostability of the droplets, Applicants split each 100-µL emulsion into 4 25-µL reactions into PCR tubes (USA Scientific, CN 1402-4700). In each reaction, 25 µL5% FC-40 oil (RAN Biotechnologies, CN 008-FluoroSurfactant-5wtF) was added to the bottom and 50 µL mineral oil (Sigma, CN M5904) was added on the top. The 2^{nd} strand cDNA synthesis and round 2 cell barcoding was performed as follows: 95° C. for 30 s, 39° C. for 5 min, 65° C. for 10 min; then 4 cycles of [98° C. for 20 s, 62° C. for 15 s, 72° C. for min], 72° C. for 5 min, hold at 4° C. Breaking emulsions and cDNA purification

After the round 2 cell barcoding was finished, the mineral oil and FC-40 oil was removed from the PCR tube, being careful and not to remove the middle layer which contains the emulsion. The emulsion was then combined and broken by adding 125 µL Recovery Agent (10x Genomics, PN 2000087). The tubes were inverted 10 times and centrifuged briefly to ensure that all droplets were broken. 125 µL Recovery Agent/Partitioning Oil (pink) from the bottom of the tube was then removed. cDNA was first purified using Dynabeads. In brief, for each reaction, a mix containing 182 µL cleanup buffer (10x Genomics, PN 2000088), 8 µL Dynabeads MyOne SILANE (10x Genomics, PN 2000048), 5 µL Reducing Agent B, and 5 µL Nuclease-free water was added. After mixing and incubating at room temperature for 10 minutes, samples were placed on a magnetic separator and washed twice with freshly prepared 80% ethanol. After removing ethanol from the second wash, each sample was eluted in 40.5 µL elution buffer that was prepared by mixing 98 µL Buffer EB (Qiagen, CN 19086), 1 µL10% Tween 20 (Teknova, CN T0710), and 1 µL Reducing Agent B. Then 40 µL of the elution was transferred to a fresh PCR tube and subjected to a 0.6x Cleanup with AMPure XP beads (Beckman Coulter, CN A63881). The cDNA was then eluted in 30 µL nuclease-free water.

### cDNA Enrichment

Before the cDNA enrichment, a qPCR reaction, containing 1 µL cDNA, 5 µL KAPA HiFi HotStart Ready Mix, 0.3 µL primer P5 (10 µM), 0.3 µL primer SMRT_PCR (10 µM) (Table 6), 2 µL 5x SYBR green (VWR, CN 12001-796), and 1.4 µL nuclease-free water, was set up to determine cycle numbers of the cDNA enrichment in a real-time thermocycler using the following program: 98° C. for 3 min, 30 cycles of [98° C. for 20 s, 67° C. for 20 s, 72° C. for 3 min], 72° C. for 5 min, hold at 4° C. The cycle numbers at which the qPCR reaction reaches early exponential amplification phase was determined as the cycle numbers for cDNA enrichment. For cDNA enrichment, 25 µL of cDNA was mixed with 125 µL KAPA HiFi HotStart Ready Mix, 7.5 µL primer P5 (10 µM), 7.5 µL primer SMRT_PCR (10 µM), and 85 µL nuclease-free water. The cDNA was enriched using the same program as the qPCR reaction, using the cycle numbers determined from the qPCR reaction. The enriched cDNA was purified using 0.6x AMPure XP beads and eluted in 50 µL nuclease-free water.

### Illumina Sequencing Library Construction

The Illumina Nextera XT DNA library preparation kit (Illumina, CN FC-131-1096) was used to prepare sequencing libraries with these following modifications: ~ 2 ng enriched cDNA and 4 µL ATM was used for each 50 µL tagmentation reaction. Following tagmentation, 2.5 µL primer P5 (5 µM) and 2.5 µL Index 1 primer (N7**, Illumina, CN FC-131-2001) was used for the PCR enrichment. The PCR reaction was removed from the thermocycler and immediately put on ice after 5 cycles of PCR amplification. Then a qPCR reaction, containing 5 µL Nextera XT PCR reaction, 3 µL NPM, 1 µL primer P5 (5 µM), 1 µL Index 1 primer, 3 µL SYBR green, and 2 µL nuclease-free water, was set up to determine the remaining cycle numbers of the library enrichment using the following program: 72° C. for 3 min, 95° C. for 30 s, 25 x [95° C. for 10 s, 55° C. for 30 s, 72° C. for 30 s], 72° C. for 5 min, hold at 10° C. The cycle numbers at which the qPCR reaction reaches one third of the fluorescence saturation was determined as the remaining cycle numbers for library enrichment. Then the remaining 45 µL library enrichment PCR reaction was put back to a thermocycler and amplified using the same program with cycle numbers determined from the qPCR. The libraries were then subjected to a 0.6x cleanup with AMPure XP beads and eluted in 25 µL nuclease-free water.

### Illumina Sequencing of BacDrop Libraries

Libraries were diluted to desired concentrations and sequenced on the Illumina NovaSeq 6000 platform with standard sequencing primers, using the following specifications: Read 1: 60 bp; Read 2: 39 bp; Index 1: 8 bp; Index 2: 16 bp. Depending the scale of the experiment and the sequencing depth desired, NovaSeq 6000 SP (Illumina #20027464), S1 (Illumina #20012865), or S2 (Illumina #20012862) reagent kits were used.

### Bulk Library Construction

To construct sequencing libraries from bacterial cultures, cell pellets collected from 1 mL early exponential phase cultures were re-suspended in 500 µL TRIzol Reagents (ThermoFisher, CN 15596026) and frozen at -80 C for at least 20 min. Cells were then thawed and mixed with 250 µL of 0.1 mm diameter Zirconia/Silica beads (BioSpec Products), and lysed mechanically via bead-beating for 90 second at 10 m/sec on a FastPrep (MP Bio). After addition of 0.1 mL chloroform, each sample tube was mixed thoroughly by inversion, incubated for 3 minutes at room temperature, and centrifuged at 12,000 xg for 15 minutes at 4° C. The aqueous phase was mixed with an equal volume of 100% ethanol, transferred to a Direct-zol spin column (Zymo Research, CN R2051), and RNA was extracted according the Direct-zol protocol. The sequencing libraries were then generated using the RNAtag-Seq protocol²⁸.

To construct sequencing libraries from fixed cells, or fixed and permeabilized cells, 20 µL cells were pelleted and resuspended 20 µL lysis buffer (50 mM Tris pH 8.0, 200 mM NaCl, 25 mM EDTA pH 8.0) supplemented with 1.6 µL proteinase K (50 mg/mL). Cells were lysed at 55° C. for 1 hour, then RNA was purified using 1.5x AMPure RNAClean XP beads (Beckman Coulter, CN A63987) and eluted in 20 µL nuclease-free water. The sequencing libraries were then generated using the RNAtag-Seq protocol.

### Estimation of mRNA Copy Numbers in E. Coli GFP Strains Using RT-qPCR

The estimation of mRNA copy numbers was performed using a protocol modified from a previous study⁶. In brief, a gfp gBlock fragment was synthesized at Integrated DNA technology. A serial dilution was performed to create gfp dsDNA standards ranging from 1 to 10¹⁰ molecules/µL. One µL of the standard was used in a 10 µL qPCR reaction to generate the standard curve. 10 ng RNA from each GFP strain (~66,666 cells with the assumption that there is 0.15 pg RNA per bacterial cell) was converted into cDNA and subjected to qPCR reaction together with the gfp dsDNA standards. GFP mRNA copy numbers were estimated by mapping to the standard curve.

### Construction of GFP Reporter Strains and Flow Cytometry

Applicants amplified the promoter region of cspD or yhcN from MGH66 and ligated it into pUA139²⁶ using the BamHI and XhoI sites, resulting in pUA139-PcspD and pUA139-PyhcN. The construct was then transformed into E.coli DH5a and the plasmids were extracted and verified using Sanger sequencing. Electrocompetent cells of MGH66 was made as previously reported²². The extracted plasmids were then transformed into MGH66 via electroporation, generating MGH66: PcspD:gfp and MGH66: PyhcN:gfp.

The cells for flow cytometry were prepared using the same scheme as the meropenem-treated sample for BacDrop. After treating with meropenem at 2 µg/mL for 30 minutes, cells were diluted into PBS at the final concentration of 10⁶ cells/mL and immediately run through flow cytometer. For the flow cytometry including dead-cell staining, 10 µL propidium iodide (1 mg/mL) (Invitrogen, cat# P1304MP) was mixed with 1 mL PBS. Cells after treatment were then diluted in the staining buffer at the final concentration of 10⁶ cells/mL, and incubated in the dark at room temperature for 15 minutes followed by running through flow cytometer. To sort GFP-low and GFP-high populations, cells were run through fluorescence-activated cell sorting (FACS). Applicants sorted ~10⁶ cells into 2 mL LB without or with meropenem (2 µg/mL) from each population. A 50 µL aliquot from cells sorted in LB medium without antibiotics was immediately diluted and plated on LB agar plates without antibiotics. The colony forming unites (CFU) on LB agar plates were enumerated to calculate the survival rates. The rest of the cells was used to measure MICs using the standard microdilution protocol²⁹ or proceeded with the persister assay. For the persister assay, cells were cultured at 37° C. with shaking. At each time points, a 50 µL aliquot was taken, diluted and plated on LB agar plates without antibiotics. Three replicates were performed in each experiment.

### RNA Fish

RNA FISH probes were designed using Design Probes tool of DECIPHER³². 3 - 5 probes were designed for each target gene (Table 11). Probes were synthesized at Integrated DNA technologies and labeled with either Cy3 or Alex488 dye at the 5′. Probes of the same gene were pooled together and diluted into 10 µM stocks. Meropenem-treated MGH66 cells were fixed and permeabilized using the same protocol as the cell fixation and permeabilization steps in BacDrop. The hybridization was carried out in 40% hybridization buffer at 50° C. overnight and the following washing steps of washing were performed as described previously³³. Cells were imaged using the DeltaVision widefield deconvolution imaging system with a 60x objective. ImageJ was used for image data analysis and cell quantification.

### Computational Methods

### Processing of Sequencing Data

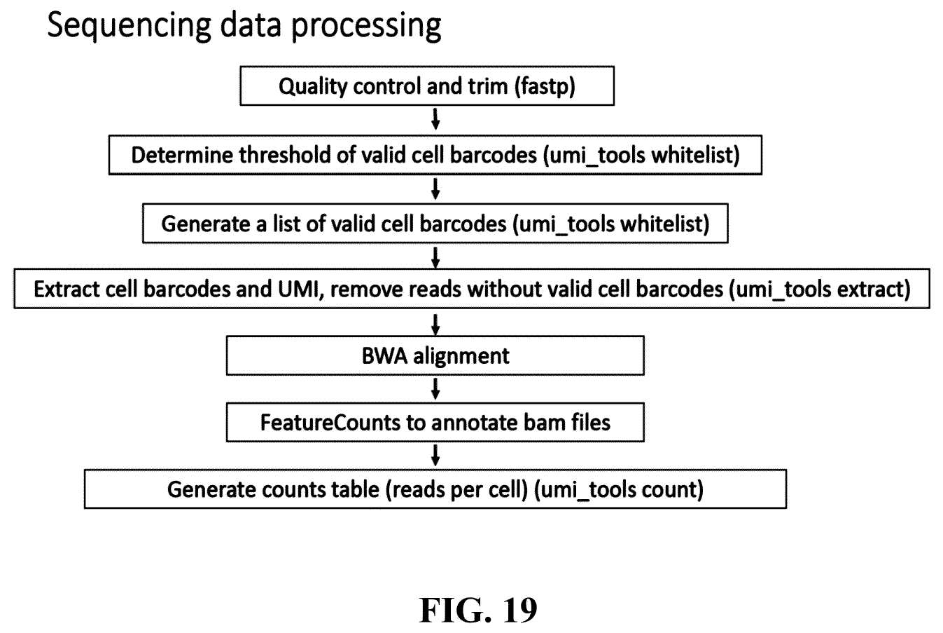

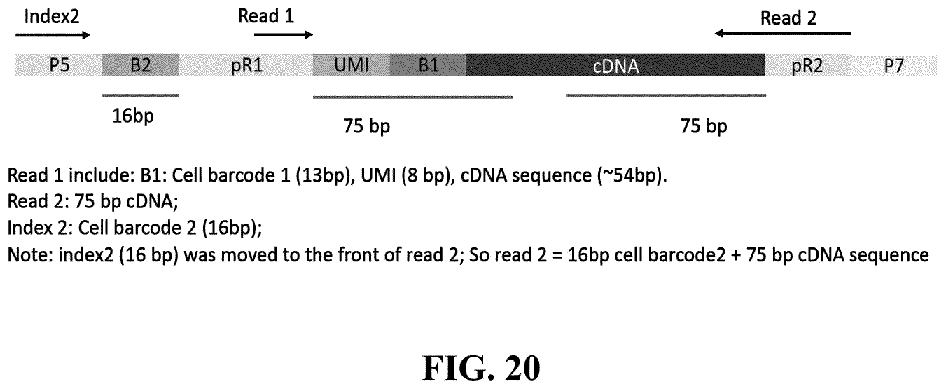

To build input matrices with gene-barcode information, Applicants created a pipeline that pulls UMI, barcodes 1 and 2 (FIG. 19). In experiments where multiple samples were pooled for droplet barcoding, a demultiplexing step based on CB1 was performed to parse different samples. Threshold of valid cell barcodes, removal invalid cell barcodes, and final count table generation was performed using UMI-tools²¹ (10.1101/gr.209601.116). Alignments were performed using BWA³⁰ and annotation of bam files were done using FeatureCounts³¹.

### Assess Potential Bias Introduced by Cell Drop-Off Caused by Insufficient Sequencing Depth

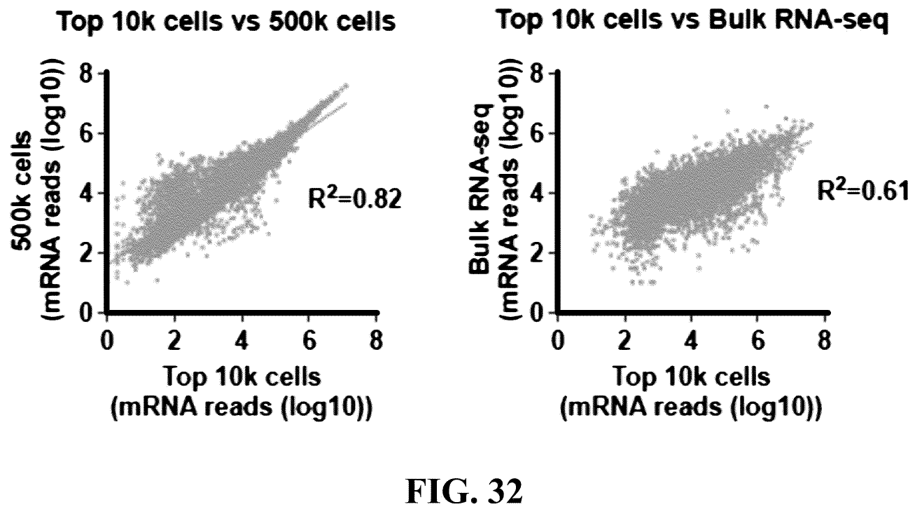

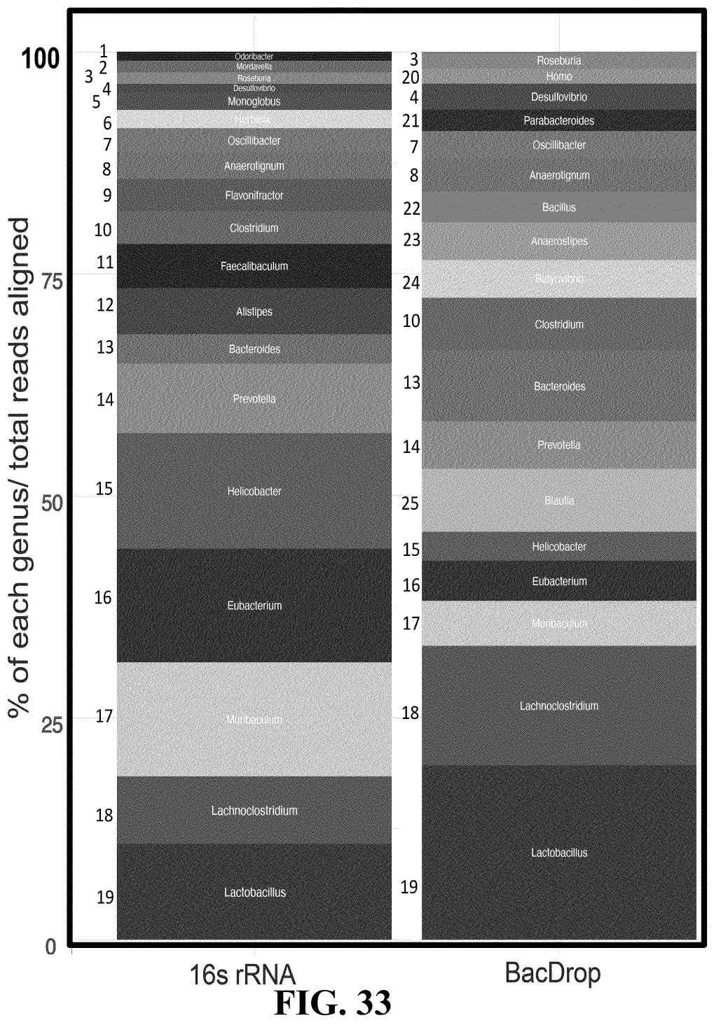

Since many cells were filtered out during the data analysis process due to low numbers of UMI, Applicants assessed the potential bias in RNA-seq results caused by cell drop-off. Applicants used the dataset from the untreated sample of K.pneumoniae MGH66 to calculate the correlation (FIG. 32). Analyzed in bulk level, Applicants found the result from top 10k cells correlates well with the top 500k cells (R²=0.82), while the correlation between top 10k cells and traditional bulk RNA-seq has a R² at 0.61.

### Analysis of BacDrop Data

Once the count tables were made, Applicants used the standard workflow of the R package Seurat 3¹⁹ (Butler A, Hoffman P, Smibert P, Papalexi E, Satija R. Integrating single-cell transcriptomic data across different conditions, technologies, and species. Nat Biotechnol. 2018;36(5):411-420; and Stuart T, Butler A, Hoffman P, et al. Comprehensive Integration of Single-Cell Data. Cell. 2019;177(7):1888-1902.e21) (v.3.2.2). Applicants excluded genes that were not expressed in any cells in the dataset, and excluded cells that had fewer than 10 or 15 genes detected and cells that had abnormally high numbers of mRNA detected. For experiments done using MGH66, Applicants identified three genes that showed consistently high-level expression in the majority of cells and in various conditions: WP-004174069.1, WP-004174069.1-2, and WP-002920103.1. Applicants use these three genes as internal controls and removed cells with a normalized expression of any of these three genes that is less than 50. The standard Seurat workflow prior to clustering was used including global normalization, feature selection, and scaling of gene expression. Applicants used the top 2000 highly variable genes as input features for clustering analysis and downstream annotation. The Seurat packages FindNeighbors and FindClusters were used for clustering at a resolution of 0.5. Uniform manifold approximation and projection (UMAP) was utilized for visualization of clustering. For the combined analysis of 4 samples of MGH66 (FIGS. 2C-2F; FIG. 11) and the analysis of 4 species (FIG. 1G; FIG. 7), both supervised and unsupervised cell clustering were performed. For the separate analysis of MGH66 samples and the BIDMC35 experiment, only unsupervised cell clustering was performed. The number of PC used in each analysis is listed in Table 7. For marker identification and annotation of clusters, Seurat’s FindMarkers tool was used with a requirement that the markers were expressed in 25% of the cells present in the dataset. For some rare population detection, the 25% criterion was removed. From the FindMarkers results, Applicants consider genes with log2 fold changes that are greater than 2, and adjusted p-value less than 0.05 as significantly differentiated genes. If a cluster did not contain any genes that pass this threshold, Applicants did not consider this as a significant cluster.

### Data Availability

Sequencing data and the processed counting matrix have been deposited to GEO repository (GSE180237).

### Example 2 – Microbiome scRNA-seq Methods

### Sample Collection

Three to four pellets of fresh murine fecal samples were taken and placed on ice until sample processing. All pellets were homogenized and a portion of the sample was used for DNA extraction and the same amount was used for BacDrop processing (see below for details).

### DNA Extraction and Library Preparation for 16S rRNA DNA Sequencing

DNA extraction was performed using the Qiagen DNeasy PowerSoil Pro Kit (HB-2495) according to manufacturer’s instructions and resuspended in 100 uL of Elution Buffer. To make the 16S library, DNA was amplified using primers targeting the V4 region:

Forward:

TCGTCGGCAGCGTCAGATGTGTATAAGAGACAGGTGYCAGCMGCCGCGGT
AA (SEQ ID NO: 1;

Reverse:

GTCTCGTGGGCTCGGAGATGTGTATAAGAGACAGGGACTACNVGGGTWTC
TAAT(SEQ ID NO: 2).

The Illumina 16S Metagenomic Sequencing Library Preparation protocol was followed according to manufacturer’s instructions and the resulting library was loaded onto an Illumina MiSeq.

### BacDrop Experimental Processing

The homogenized sample was washed once with 1mL of ice cold 1 × PBS + 4% Formaldehyde. After spin down, the sample was resuspended in 7 ml of ice cold 1 × PBS + 4% Formaldehyde and placed at 4C rotating overnight. The remainder of the protocol follows the standard BacDrop experimental set up.

### Data Analysis

Initial data processing with fastp remained the same as previously described for both the BacDrop and 16S libraries. The resulting reads were used as input in the Kraken2 (genomebiology.biomedcentral.com/articles/10.1186/s13059-019-1891-0) taxonomy identification pipeline. The Kraken2 report was analyzed using Pavian (academic.oup.com/bioinformatics/article/36/4/1303/5573755?login=false), and resulting genus comparisons were calculating using the abundances of reads in each genus. The percent was calculated by first normalizing the data from each library by the total number of reads in order to compare across libraries.

### Results

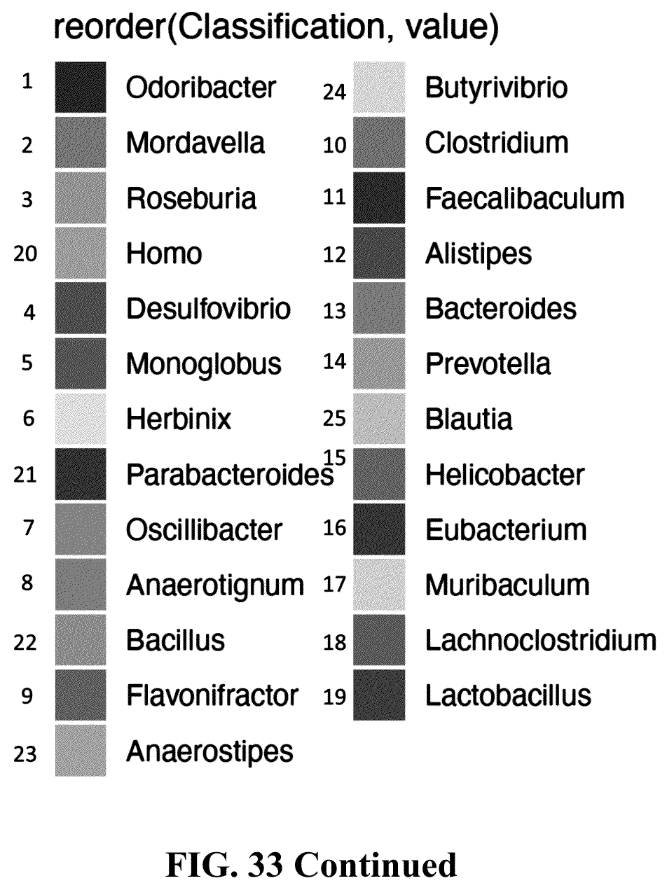

Applicants captured 127 genera using BacDrop, whereas only 58 genera were captured using 16s rRNA DNA sequencing (FIG. 33; Tables 12 and 13). 54 genera were captured by both methods.

### REFERENCES

1 Ackermann, M. A functional perspective on phenotypic heterogeneity in microorganisms. Nat Rev Microbiol 13, 497-508, doi:10.1038/nrmicro3491 (2015).
2 Davis, K. M. & Isberg, R. R. Defining heterogeneity within bacterial populations via single cell approaches. Bioessays 38, 782-790, doi:10.1002/bies.201500121 (2016).
3 Potter, S. S. Single-cell RNA sequencing for the study of development, physiology and disease. Nat Rev Nephrol 14, 479-492, doi:10.1038/s41581-018-0021-7 (2018).
4 Stuart, T. & Satija, R. Integrative single-cell analysis. Nat Rev Genet 20, 257-272, doi:10.1038/s41576-019-0093-7 (2019).
5 Imdahl, F. & Saliba, A. E. Advances and challenges in single-cell RNA-seq of microbial communities. Curr Opin Microbiol 57, 102-110, doi:10.1016/j.mib.2020.10.001 (2020).
6 Taniguchi, Y. et al. Quantifying E. coli proteome and transcriptome with single-molecule sensitivity in single cells. Science 329, 533-538, doi:10.1126/science.1188308 (2010).
7 Blattman, S. B., Jiang, W., Oikonomou, P. & Tavazoie, S. Prokaryotic single-cell RNA sequencing by in situ combinatorial indexing. Nat Microbiol 5, 1192-1201, doi:10.1038/s41564-020-0729-6 (2020).
8 Imdahl, F., Vafadarnejad, E., Homberger, C., Saliba, A. E. & Vogel, J. Single-cell RNA-sequencing reports growth-condition-specific global transcriptomes of individual bacteria. Nat Microbiol 5, 1202-1206, doi: 10.1038/s41564-020-0774-1 (2020).
9 Kuchina, A. et al. Microbial single-cell RNA sequencing by split-pool barcoding. Science 371, doi:10.1126/science.aba5257 (2021).
10 Dar, D., Dar, N., Cai, L. & Newman, D. K. Spatial transcriptomics of planktonic and sessile bacterial populations at single-cell resolution. Science 373, doi:10.1126/science.abi4882 (2021).
11 McNulty, R. S., Duluxan; Liu, Shichen; Hormoz, Sahand; Rosenthal, Adam Z.. Droplet-based single cell RNA sequencing of bacteria identifies known and previously unseen cellular states. bioRxiv preprint, doi:https://doi.org/10.1101/2021.03.10.434868 (2021).12 Dewachter, L., Fauvart, M. & Michiels, J. Bacterial Heterogeneity and Antibiotic Survival: Understanding and Combatting Persistence and Heteroresistance. Mol Cell 76, 255-267, doi:10.1016/j.molcel.2019.09.028 (2019).
13 Andersson, D. I., Nicoloff, H. & Hjort, K. Mechanisms and clinical relevance of bacterial heteroresistance. Nat Rev Microbiol 17, 479-496, doi:10.1038/s41579-019-0218-1 (2019).
14 Centers for Disease Control and Prevention: Antibiotic Resistance Threats in the United States. doi:https://www.cdc.gov/drugresistance/pdf/threatsreport/2019-ar-threats-report-508.pdf (2019).15 Kim, Y. & Wood, T. K. Toxins Hha and CspD and small RNA regulator Hfq are involved in persister cell formation through MqsR in Escherichia coli. Biochem Biophys Res Commun 391, 209-213, doi:10.1016/j.bbrc.2009.11.033 (2010).
16 Yamanaka, K., Zheng, W., Crooke, E., Wang, Y. H. & Inouye, M. CspD, a novel DNA replication inhibitor induced during the stationary phase in Escherichia coli. Mol Microbiol 39, 1572-1584, doi:10.1046/j.1365-2958.2001.02345.x (2001).
17 Macosko, E. Z. et al. Highly Parallel Genome-wide Expression Profiling of Individual Cells Using Nanoliter Droplets. Cell 161, 1202-1214, doi:10.1016/j.cell.2015.05.002 (2015).
18 Datlinger, P. et al. Ultra-high-throughput single-cell RNA sequencing and perturbation screening with combinatorial fluidic indexing. Nat Methods 18, 635-642, doi:10.1038/s41592-021-01153-z (2021).
19 Stuart, T. et al. Comprehensive Integration of Single-Cell Data. Cell 177, 1888-1902 e1821, doi:10.1016/j.cell.2019.05.031 (2019).
20 Bhattacharyya, R. P. et al. Simultaneous detection of genotype and phenotype enables rapid and accurate antibiotic susceptibility determination. Nat Med 25, 1858-1864, doi:10.1038/s41591-019-0650-9 (2019).
21 Smith, T., Heger, A. & Sudbery, I. UMI-tools: modeling sequencing errors in Unique Molecular Identifiers to improve quantification accuracy. Genome Res 27, 491-499, doi:10.1101/gr.209601.116 (2017).
22 Ma, P. et al. Genetic determinants facilitating the evolution of resistance to carbapenem antibiotics. Elife 10, doi:10.7554/eLife.67310 (2021).
23 Ma, P., Laibinis, H. H., Ernst, C. M. & Hung, D. T. Carbapenem Resistance Caused by High-Level Expression of OXA-663 beta-Lactamase in an OmpK36-Deficient Klebsiella pneumoniae Clinical Isolate. Antimicrob Agents Chemother 62, doi:10.1128/AAC.01281-18 (2018).
24 Cho, H., Uehara, T. & Bernhardt, T. G. Beta-lactam antibiotics induce a lethal malfunctioning of the bacterial cell wall synthesis machinery. Cell 159, 1300-1311, doi:10.1016/j.cell.2014.11.017 (2014).
25 Van Dyk, T. K., DeRose, E. J. & Gonye, G. E. LuxArray, a high-density, genomewide transcription analysis of Escherichia coli using bioluminescent reporter strains. J Bacteriol 183, 5496-5505, doi:10.1128/JB.183.19.5496-5505.2001 (2001).
26 Zaslaver, A. et al. A comprehensive library of fluorescent transcriptional reporters for Escherichia coli. Nat Methods 3, 623-628, doi:10.1038/nmeth895 (2006).
27 Harkness, R. E., Kusser, W., Qi, B. J. & Ishiguro, E. E. Genetic mapping of the lytA and lytB loci of Escherichia coli, which are involved in penicillin tolerance and control of the stringent response. Can J Microbiol 38, 975-978, doi:10.1139/m92-156 (1992).
28 Shishkin, A. A. et al. Simultaneous generation of many RNA-seq libraries in a single reaction. Nat Methods 12, 323-325, doi:10.1038/nmeth.3313 (2015).
29 Wiegand, I., Hilpert, K. & Hancock, R. E. Agar and broth dilution methods to determine the minimal inhibitory concentration (MIC) of antimicrobial substances. Nat Protoc 3, 163-175, doi:10.1038/nprot.2007.521 (2008).
30 Li, H. & Durbin, R. Fast and accurate short read alignment with Burrows-Wheeler transform. Bioinformatics 25, 1754-1760, doi:10.1093/bioinformatics/btp324 (2009).
31 Liao, Y., Smyth, G. K. & Shi, W. featureCounts: an efficient general purpose program for assigning sequence reads to genomic features. Bioinformatics 30, 923-930, doi:10.1093/bioinformatics/btt656 (2014).
32 Wright, E. S., Yilmaz, L. S., Corcoran, A. M., Okten, H. E. & Noguera, D. R. Automated design of probes for rRNA-targeted fluorescence in situ hybridization reveals the advantages of using dual probes for accurate identification. Appl Environ Microbiol 80, 5124-5133, doi:10.1128/AEM.01685-14 (2014).
33 Skinner, S. O., Sepulveda, L. A., Xu, H. & Golding, I. Measuring mRNA copy number in individual Escherichia coli cells using single-molecule fluorescent in situ hybridization. Nat Protoc 8, 1100-1113, doi:10.1038/nprot.2013.066 (2013).
34 Dar, D. D., Nina; Cai, Long; Newman, K. Dianne. In situ single-cell activities of microbial populations revealed by spatial transcriptomics bioRxiv preprint, doi.org/10.1101/2021.02.24.432792 (2021).

### TABLES

TABLE 1

RT Primers

| | | | | |
|---|---|---|---|---|
| Primer ID | plate_name | plate_ well | CB1 sequence | oligo_seq[5′>3′] |
| P1_01 | BacDrop_RT10X_384_plate1 | A01 | AAGTGATTAGCAA (SEQ ID NO: 3) | TCGTCGGCAGCGTCAGATGTGTATAAGAGACAGNNNNNNNNAAGTGATTAGCAANNNNNN (SEQ ID NO: 4) |
| P1_02 | BacDrop_RT10X_384_plate1 | A02 | AGAATCCCCCTAA (SEQ ID NO: 5) | TCGTCGGCAGCGTCAGATGTGTATAAGAGACAGNNNNNNNNAGAATCCCCCTAANNNNNN (SEQ ID NO: 6) |
| P1_03 | BacDrop_RT10X_384_plate1 | A03 | ACCTGGGAAACTA (SEQ ID NO: 7) | TCGTCGGCAGCGTCAGATGTGTATAAGAGACAGNNNNNNNNACCTGGGAAACTANNNNNN (SEQ ID NO: 8) |
| P1_04 | BacDrop_RT10X_384_platel | A04 | ATACCTCCCAGGA (SEQ ID NO: 9) | TCGTCGGCAGCGTCAGATGTGTATAAGAGACAGNNNNNNNNATACCTCCCAGGANNNNNN (SEQ ID NO: 10) |
| P1_05 | BacDrop_RT10X_384_platel | A05 | AATTTGTGGTATA (SEQ ID NO: 11) | TCGTCGGCAGCGTCAGATGTGTATAAGAGACAGNNNNNNNNAATTTGTGGTATANNNNNN (SEQ ID NO: 12) |
| P1_06 | BacDrop_RT10X_384_platel | A06 | ACCCGAGAGATCA (SEQ ID NO: 13) | TCGTCGGCAGCGTCAGATGTGTATAAGAGACAGNNNNNNNNACCCGAGAGATCANNNNNN (SEQ ID NO: 14) |
| P1_07 | BacDrop_RT10X_384_platel | A07 | AGAGTATAGGGTA (SEQ ID NO: 15) | TCGTCGGCAGCGTCAGATGTGTATAAGAGACAGNNNNNNNNAGAGTATAGGGTANNNNNN (SEQ ID NO: 16) |
| P1_08 | BacDrop_RT10X_384_plate1 | A08 | ATCTTAATTGAGA (SEQ ID NO: 17) | TCGTCGGCAGCGTCAGATGTGTATAAGAGACAGNNNNNNNNATCTTAATTGAGANNNNNN (SEQ ID NO: 18) |
| P1_09 | BacDrop_RT10X_384_plate1 | A09 | AACGTTCTGTCGA (SEQ ID NO: 19) | TCGTCGGCAGCGTCAGATGTGTATAAGAGACACNNNNNNNNAACGTTCTGTCGANNNNNN (SEQ ID NO: 20) |
| P1_10 | BacDrop_RT10X_384_plate1 | A10 | ACATAATGGCGAA (SEQ ID NO: 21) | TCGTCGGCAGCGTCAGATGTGTATAAGAGACAGNNNNNNNNACATAATGGCGAANNNNNN (SEQ ID NO: 22) |
| P1_11 | BacDrop_RT10X_384_plate1 | A11 | AGGCCGGAACATA (SEQ ID NO: 23) | TCGTCGGCAGCGTCAGATGTGTATAAGAGACAGNNNNNNNNAGGCCGGAACATANNNNNN (SEQ ID NO: 24) |
| P1_12 | BacDrop_RT10X_384_plate1 | A12 | ATCACTTCAAGAA (SEQ ID NO: 25) | TCGTCGGCAGCGTCAGATGTGTATAAGAGACAGNNNNNNNNATCACTTCAAGAANNNNNN (SEQ ID NO: 26) |
| P1_13 | BacDrop_RT10X_384_plate1 | B01 | AAGCTGGCCTTCA (SEQ ID NO: 27) | TCGTCGGCAGCGTCAGATGTGTATAAGAGACAGNNNNNNNNAAGCTGGCCTTCANNNNNN (SEQ ID NO: 28) |
| P1_14 | BacDrop_RT10X_384_platel1 | B02 | ACTGCACATTGGA (SEQ ID NO: 29) | TCGTCGGCAGCGTCAGATGTGTATAAGAGACAGNNNNNNNNACTGCACATTGGANNNNNN (SEQ ID NO: 30) |
| P1_15 | BacDrop_RT10X_384_plate1 | B03 | AGGTCCTTCGATA (SEQ ID NO: 31) | TCGTCGGCAGCGTCAGATGTGTATAAGAGACAGNNNNNNNNAGGTCCTTCGATANNNNNN (SEQ ID NO: 32) |
| P1_16 | BacDrop_RT10X_384_plate1 | B04 | ATTCGCACCCGAA (SEQ ID NO: 33) | TCGTCGGCAGCGTCAGATGTGTATAAGAGACAGNNNNNNNNATTCGCACCCGAANNNNNN (SEQ ID NO: 34) |
| P1_17 | BacDrop_RT10X_384_plate1 | B05 | ACCGTAAATTACA (SEQ ID NO: 35) | TCGTCGGCAGCGTCAGATGTGTATAAGAGACAGNNNNNNNNACCGTAAATTACANNNNNN (SEQ ID NO: 36) |
| P1_18 | BacDrop_RT10X_384_plate1 | B06 | AGTAGTCGATATA (SEQ ID NO: 37) | TCGTCGGCAGCGTCAGATGTGTATAAGAGACAGNNNNNNNNAGTAGTCGATATANNNNNN (SEQ ID NO: 38) |
| P1_19 | BacDrop_RT10X_384_plate1 | B07 | ATGTAAGAATCAA (SEQ ID NO: 39) | TCGTCGGCAGCGTCAGATGTGTATAAGAGACAGNNNNNNNNATGTAAGAATCAANNNNNN (SEQ ID NO: 40) |
| P1_20 | BacDrop_RT10X_384_plate1 | B08 | AAGGCCCGCCGTA (SEQ ID NO: 41) | TCGTCGGCAGCGTCAGATGTGTATAAGAGACAGNNNNNNNNAAGGCCCGCCGTANNNNNN (SEQ ID NO: 42) |
| P1_21 | BacDrop_RT10X_384_plate1 | B09 | ACTCCACGCAAGA (SEQ ID NO: 43) | TCGTCGGCAGCGTCAGATGTGTATAAGAGACAGNNNNNNNNACTCCACGCAAGANNNNNN (SEQ ID NO: 44) |
| P1_22 | BacDrop_RT10X_384_plate1 | B10 | AGTCTTTGATTAA (SEQ ID NO: 45) | TCGTCGGCAGCGTCAGATGTGTATAAGAGACAGNNNNNNNNAGTCTTTGATTAANNNNNN (SEQ ID NO: 46) |
| P1_23 | BacDrop_RT10X_384_plate1 | B11 | ATAGAGCCCGCCA (SEQ ID NO: 47) | TCGTCGGCAGCGTCAGATGTGTATAAGAGACAGNNNNNNNNATAGAGCCCGCCANNNNNN (SEQ ID NO: 48) |
| P1_24 | BacDrop_RT10X_384_plate1 | B12 | AAGAAATGGAGGA (SEQ ID NO: 49) | TCGTCGGCAGCGTCAGATGTGTATAAGAGACAGNNNNNNNNAAGAAATGGAGGANNNNNN (SEQ ID NO: 50) |
| P1_25 | BacDrop_RT10X_384_plate1 | C01 | TCAGCGCTGGTTA (SEQ ID NO: 51) | TCGTCGGCAGCGTCAGATGTGTATAAGAGACAGNNNNNNNNTCAGCGCTGGTTANNNNNN (SEQ ID NO: 52) |
| P1_26 | BacDrop_RT10X_384_plate1 | C02 | TGTTGACGAATAA (SEQ ID NO: 53) | TCGTCGGCAGCGTCAGATGTGTATAAGAGACAGNNNNNNNNTGTTGACGAATAANNNNNN (SEQ ID NO: 54) |
| P1_27 | BacDrop_RT10X_384_plate1 | C03 | TTGCATCAGCGCA (SEQ ID NO: 55) | TCGTCGGCAGCGTCAGATGTGTATAAGAGACAGNNNNNNNNTTGCATCAGCGCANNNNNN (SEQ ID NO: 56) |
| P1_28 | BacDrop_RT10X_384_plate1 | C04 | TAGCCATATGAGA (SEQ ID NO: 57) | TCGTCGGCAGCGTCAGATGTGTATAAGAGACAGNNNNNNNNTAGCCATATGAGANNNNNN (SEQ ID NO: 58) |
| P1_29 | BacDrop_RT10X_384_plate1 | C05 | TCCTGCCCCTGTA (SEQ ID NO: 59) | TCGTCGGCAGCGTCAGATGTGTATAAGAGACAGNNNNNNNNTCCTGCCCCTGTANNNNNN (SEQ ID NO: 60) |
| P1_30 | BacDrop_RT10X_384_plate1 | C06 | TGGTATAAAACCA (SEQ ID NO: 61) | TCGTCGGCAGCGTCAGATGTGTATAAGAGACAGNNNNNNNNTGGTATAAAACCANNNNNN (SEQ ID NO: 62) |
| P1_31 | BacDrop_RT10X_384_plate1 | C07 | TTAAAGTCATGCA (SEQ ID NO: 63) | TCGTCGGCAGCGTCAGATGTGTATAAGAGACAGNNNNNNNNTTAAAGTCATGCANNNNNN (SEQ ID NO: 64) |
| P1_32 | BacDrop_RT10X_384_plate1 | C08 | TAGAACTCAGAAA (SEQ ID NO: 65) | TCGTCGGCAGCGTCAGATGTGTATAAGAGACAGNNNNNNNNTAGAACTCAGAAANNNNNN (SEQ ID NO: 66) |
| P1_33 | BacDrop_RT10X_384_plate1 | C09 | TCAGCTTAATCGC (SEQ ID NO: 67) | TCGTCGGCAGCGTCAGATGTGTATAAGAGACAGNNNNNNNNTCAGCTTAATCGCNNNNNN (SEQ ID NO: 68) |
| P1_34 | BacDrop_RT10X_384_plate1 | C10 | TGGCTTAAGAGAC (SEQ ID NO: 69) | TCGTCGGCAGCGTCAGATGTGTATAAGAGACAGNNNNNNNNTGGCTTAAGAGACNNNNNN (SEQ ID NO: 70) |
| P1_35 | BacDrop_RT10X_384_plate1 | C11 | TTCACCTGTCGGC (SEQ ID NO: 71) | TCGTCGGCAGCGTCAGATGTGTATAAGAGACAGNNNNNNNNTTCACCTGTCGGCNNNNNN (SEQ ID NO: 72) |
| P1_36 | BacDrop_RT10X_384_plate1 | C12 | TATAGTGATACTC (SEQ ID NO: 73) | TCGTCGGCAGCGTCAGATGTGTATAAGAGACAGNNNNNNNNTATAGTGATACTCNNNNNN (SEQ ID NO: 74) |
| P1_37 | BacDrop_RT10X_384_plate1 | D01 | TCATCAGCGAACC (SEQ ID NO: 75) | TCGTCGGCAGCGTCAGATGTGTATAAGAGACAGNNNNNNNNTCATCAGCGAACCNNNNNN (SEQ ID NO: 76) |
| P1_38 | BacDrop_RT10X_384_plate1 | D02 | TGCGCCATGGGAC (SEQ ID NO: 77) | TCGTCGGCAGCGTCAGATGTGTATAAGAGACAGNNNNNNNNTGCGCCATGGGACNNNNNN (SEQ ID NO: 78) |
| P1_39 | BacDrop_RT10X_384_plate1 | D03 | TTGTTGAACTCCC (SEQ ID NO: 79) | TCGTCGGCAGCGTCAGATGTGTATAAGAGACAGNNNNNNNNTTGTTGAACTCCCNNNNNN (SEQ ID NO: 80) |
| P1_40 | BacDrop_RT10X_384_plate1 | D04 | TATATACGCAGTC (SEQ ID NO: 81) | TCGTCGGCAGCGTCAGATGTGTATAAGAGACAGNNNNNNNNTATATACGCAGTCNNNNNN (SEQ ID NO: 82) |
| P1_41 | BacDrop_RT10X_384_plate1 | D05 | TCGCACGACGTAC (SEQ ID NO: 83) | TCGTCGGCAGCGTCAGATGTGTATAAGAGACAGNNNNNNNNTCGCACGACGTACNNNNNN (SEQ ID NO: 84) |
| P1_42 | BacDrop_RT10X_384_plate1 | D06 | TGGGACAGAAGGC (SEQ ID NO: 85) | TCGTCGGCAGCGTCAGATGTGTATAAGAGACAGNNNNNNNNTGGGACAGAAGGCNNNNNN (SEQ ID NO: 86) |
| P1_43 | BacDrop_RT10X_384_plate1 | D07 | TTTAACAAGTATC (SEQ ID NO: 87) | TCGTCGGCAGCGTCAGATGTGTATAAGAGACAGNNNNNNNNTTTAACAAGTATCNNNNNN (SEQ ID NO: 88) |
| P1_44 | BacDrop_RT10X_384_plate1 | D08 | TACTCCTGGGTAC (SEQ ID NO: 89) | TCGTCGGCAGCGTCAGATGTGTATAAGAGACAGNNNNNNNNTACTCCTGGGTACNNNNNN (SEQ ID NO: 90) |
| P1_45 | BacDrop_RT10X_384_plate1 | D09 | TCAGAAAACCCCC (SEQ ID NO: 91) | TCGTCGGCAGCGTCAGATGTGTATAAGAGACAGNNNNNNNNTCAGAAAACCCCCNNNNNN (SEQ ID NO: 92) |
| P1_46 | BacDrop_RT10X_384_plate1 | D10 | TGCACGAGCAGTC (SEQ ID NO: 93) | TCGTCGGCAGCGTCAGATGTGTATAAGAGACAGNNNNNNNNTGCACGAGCAGTCNNNNNN (SEQ ID NO: 94) |
| P1_47 | BacDrop_RT10X_384_plate1 | D11 | TTATTATGCTTCC (SEQ ID NO: 95) | TCGTCGGCAGCGTCAGATGTGTATAAGAGACAGNNNNNNNNTTATTATGCTTCCNNNNNN (SEQ ID NO: 96) |
| P1_48 | BacDrop_RT10X_384_plate1 | D12 | TAGCGACTGTATC (SEQ ID NO: 97) | TCGTCGGCAGCGTCAGATGTGTATAAGAGACAGNNNNNNNNTAGCGACTGTATCNNNNNN (SEQ ID NO: 98) |
| P1_49 | BacDrop_RT10X_384_plate1 | E01 | CCCTTGCGACAGC (SEQ ID NO: 99) | TCGTCGGCAGCGTCAGATGTGTATAAGAGACAGNNNNNNNNCCCTTGCGACAGCNNNNNN (SEQ ID NO: 100) |
| P1_50 | BacDrop_RT10X_384_plate1 | E02 | CGTTAGATTGCAC (SEQ ID NO: 101) | TCGTCGGCAGCGTCAGATGTGTATAAGAGACAGNNNNNNNNCGTTAGATTGCACNNNNNN (SEQ ID NO: 102) |
| P1_51 | BacDrop_RT10X_384_plate1 | E03 | CTAAATACCATCC (SEQ ID NO: 103) | TCGTCGGCAGCGTCAGATGTGTATAAGAGACAGNNNNNNNNCTAAATACCATCCNNNNNN (SEQ ID NO: 104) |
| P1_52 | BacDrop_RT10X_384_plate1 | E04 | CATATTTTTTAGC (SEQ ID NO: 105) | TCGTCGGCAGCGTCAGATGTGTATAAGAGACAGNNNNNNNNCATATTTTTTAGCNNNNNN (SEQ ID NO: 106) |
| P1_53 | BacDrop_RT10X_384_plate1 | E05 | CCCCTAAACGCTC (SEQ ID NO: 107) | TCGTCGGCAGCGTCAGATGTGTATAAGAGACAGNNNNNNNNCCCCTAAACGCTCNNNNNN (SEQ ID NO: 108) |
| P1_54 | BacDrop_RT10X_384_plate1 | E06 | CGATTGTTAGGAC (SEQ ID NO: 109) | TCGTCGGCAGCGTCAGATGTGTATAAGAGACAGNNNNNNNNCGATTGTTAGGACNNNNNN (SEQ ID NO: 110) |
| P1_55 | BacDrop_RT10X_384_plate1 | E07 | CTACCGAGTCTCC (SEQ ID NO: 111) | TCGTCGGCAGCGTCAGATGTGTATAAGAGACAGNNNNNNNNCTACCGAGTCTCCNNNNNN (SEQ ID NO: 112) |
| P1_56 | BacDrop_RT10X_384_plate1 | E08 | CAGAACAAATTGC (SEQ ID NO: 113) | TCGTCGGCAGCGTCAGATGTGTATAAGAGACAGNNNNNNNNCAGAACAAATTGCNNNNNN (SEQ ID NO: 114) |
| P1_57 | BacDrop_RT10X_384_plate1 | E09 | CCTTGATCTCATC (SEQ ID NO: 115) | TCGTCGGCAGCGTCAGATGTGTATAAGAGACAGNNNNNNNNCCTTGATCTCATCNNNNNN (SEQ ID NO: 116) |
| P1_58 | BacDrop_RT10X_384_plate1 | E10 | CGCCACGCGTTAC (SEQ ID NO: 117) | TCGTCGGCAGCGTCAGATGTGTATAAGAGACAGNNNNNNNNCGCCACGCGTTACNNNNNN (SEQ ID NO: 118) |
| P1_59 | BacDrop_RT10X_384_plate1 | E11 | CTATAGATACGGC (SEQ ID NO: 119) | TCGTCGGCAGCGTCAGATGTGTATAAGAGACAGNNNNNNNNCTATAGATACGGCNNNNNN (SEQ ID NO: 120) |
| P1_60 | BacDrop_RT10X_384_plate1 | E12 | CATACCCAGCCAC (SEQ ID NO: 121) | TCGTCGGCAGCGTCAGATGTGTATAAGAGACAGNNNNNNNNCATACCCAGCCACNNNNNN (SEQ ID NO: 122) |
| P1_61 | BacDrop_RT10X_384_plate1 | F01 | CCCCGGATGTACC (SEQ ID NO: 123) | TCGTCGGCAGCGTCAGATGTGTATAAGAGACAGNNNNNNNNCCCCGGATGTACCNNNNNN (SEQ ID NO: 124) |
| P1_62 | BacDrop_RT10X_384_plate1 | F02 | CGATCTCGGCTTC (SEQ ID NO: 125) | TCGTCGGCAGCGTCAGATGTGTATAAGAGACAGNNNNNNNNCGATCTCGGCTTCNNNNNN (SEQ ID NO: 126) |
| P1_63 | BacDrop_RT10X_384_plate1 | F03 | CTAGCCGCTTTGC (SEQ ID NO: 127) | TCGTCGGCAGCGTCAGATGTGTATAAGAGACAGNNNNNNNNCTAGCCGCTTTGCNNNNNN (SEQ ID NO: 128) |
| P1_64 | BacDrop_RT10X_384_plate1 | F04 | CCTAAATACAGCC (SEQ ID NO: 129) | TCGTCGGCAGCGTCAGATGTGTATAAGAGACAGNNNNNNNNCCTAAATACAGCCNNNNNN (SEQ ID NO: 130) |
| P1_65 | BacDrop_RT10X_384_plate1 | F05 | CGGGTTTTACCAG (SEQ ID NO: 131) | TCGTCGGCAGCGTCAGATGTGTATAAGAGACAGNNNNNNNNCGGGTTTTACCAGNNNNNN (SEQ ID NO: 132) |
| P1_66 | BacDrop_RT10X_384_plate1 | F06 | CATATCCATTGCG (SEQ ID NO: 133) | TCGTCGGCAGCGTCAGATGTGTATAAGAGACAGNNNNNNNNCATATCCATTGCGNNNNNN (SEQ ID NO: 134) |
| P1_67 | BacDrop_RT10X_384_plate1 | F07 | CCTCCCGAGAGTG (SEQ ID NO: 135) | TCGTCGG CAGCGTCAGATGTGTATAAGAGACAG N N N N N N N N CCTCCCGAGAGTG N N N N N N (SEQ ID NO: 136) |
| P1_68 | BacDrop_RT10X_384_plate1 | F08 | CGAATATCTCCGG (SEQ ID NO: 137) | TCGTCGGCAGCGTCAGATGTGTATAAGAGACAGNNNNNNNNCGAATATCTCCGGNNNNNN (SEQ ID NO: 138) |
| P1_69 | BacDrop_RT10X_384_plate1 | F09 | CTACTAACCCACG (SEQ ID NO: 139) | TCGTCGGCAGCGTCAGATGTGTATAAGAGACAGNNNNNNNNCTACTAACCCACGNNNNNN (SEQ ID NO: 140) |
| P1_70 | BacDrop_RT10X_384_plate1 | F10 | CAAATGATACAAG (SEQ ID NO: 141) | TCGTCGG CAGCGTCAGATGTGTATAAGAGACAG N N N N N N N N CAAATGATACAAG N N N N N N (SEQ ID NO: 142) |
| P1_71 | BacDrop_RT10X_384_plate1 | F11 | CCCCCATTATTTG (SEQ ID NO: 143) | TCGTCGG CAGCGTCAGATGTGTATAAGAGACAGNNNNNNNNCCCCCATTATTTGNNNNNN (SEQ ID NO: 144) |
| P1_72 | BacDrop_RT10X_384_plate1 | F12 | CGAGTCGCGACGG (SEQ ID NO: 145) | TCGTCGGCAGCGTCAGATGTGTATAAGAGACAGNNNNNNNNCGAGTCGCGACGGNNNNNN (SEQ ID NO: 146) |
| P1_73 | BacDrop_RT10X_384_plate1 | G01 | GTGGTTGTGGCCG (SEQ ID NO: 147) | TCGTCGGCAGCGTCAGATGTGTATAAGAGACAGNNNNNNNNGTGGTTGTGGCCGNNNNNN (SEQ ID NO: 148) |
| P1_74 | BacDrop_RT10X_384_plate1 | G02 | GATTCAAGGAAAG (SEQ ID NO: 149) | TCGTCGGCAGCGTCAGATGTGTATAAGAGACAGNNNNNNNNGATTCAAGGAAAGNNNNNN (SEQ ID NO: 150) |
| P1_75 | BacDrop_RT10X_384_plate1 | G03 | GCCAAGATCTGGG (SEQ ID NO: 151) | TCGTCGGCAGCGTCAGATGTGTATAAGAGACAGNNNNNNNNGCCAAGATCTGGGNNNNNN (SEQ ID NO: 152) |
| P1_76 | BacDrop_RT10X_384_plate1 | G04 | GATACTGTATCAG (SEQ ID NO: 153) | TCGTCGGCAGCGTCAGATGTGTATAAGAGACAGNNNNNNNNGATACTGTATCAGNNNNNN (SEQ ID NO: 154) |
| P1_77 | BacDrop_RT10X_384_platel | G05 | GCCGATTCGTGCG (SEQ ID NO: 155) | TCGTCGGCAGCGTCAGATGTGTATAAGAGACAGNNNNNNNNGCCGATTCGTGCGNNNNNN (SEQ ID NO: 156) |
| P1_78 | BacDrop_RT10X_384_plate1 | G06 | GTGACAACGCTGG (SEQ ID NO: 157) | TCGTCGGCAGCGTCAGATGTGTATAAGAGACAGNNNNNNNNGTGACAACGCTGGNNNNNN (SEQ ID NO: 158) |
| P1_79 | BacDrop_RT10X_384_plate1 | G07 | GGGCTGTCTTATG (SEQ ID NO: 159) | TCGTCGGCAGCGTCAGATGTGTATAAGAGACAGNNNNNNNNGGGCTGTCTTATGNNNNNN (SEQ ID NO: 160) |
| P1_80 | BacDrop_RT10X_384_plate1 | G08 | GACCCTGGCCTGG (SEQ ID NO: 161) | TCGTCGGCAGCGTCAGATGTGTATAAGAGACAGNNNNNNNNGACCCTGGCCTGGNNNNNN (SEQ ID NO: 162) |
| P1_81 | BacDrop_RT10X_384_plate1 | G09 | GCGTACGCTTATG (SEQ ID NO: 163) | TCGTCGGCAGCGTCAGATGTGTATAAGAGACAGNNNNNNNNGCGTACGCTTATGNNNNNN (SEQ ID NO: 164) |
| P1_82 | BacDrop_RT10X_384_plate1 | G10 | GTTGCACTTAATG (SEQ ID NO: 165) | TCGTCGGCAGCGTCAGATGTGTATAAGAGACAGNNNNNNNNGTTGCACTTAATGNNNNNN (SEQ ID NO: 166) |
| P1_83 | BacDrop_RT10X_384_plate1 | G11 | GGACAAAGAGAAG (SEQ ID NO: 167) | TCGTCGGCAGCGTCAGATGTGTATAAGAGACAGNNNNNNNNGGACAAAGAGAAGNNNNNN (SEQ ID NO: 168) |
| P1_84 | BacDrop_RT10X_384_plate1 | G12 | GAACCACAGCACG (SEQ ID NO: 169) | TCGTCGGCAGCGTCAGATGTGTATAAGAGACAGNNNNNNNNGAACCACAGCACGNNNNNN (SEQ ID NO: 170) |
| P1_85 | BacDrop_RT10X_384_plate1 | H01 | GCTTATCTAGAGG (SEQ ID NO: 171) | TCGTCGGCAGCGTCAGATGTGTATAAGAGACAGNNNNNNNNGCTTATCTAGAGGNNNNNN (SEQ ID NO: 172) |
| P1_86 | BacDrop_RT10X_384_plate1 | H02 | GTAATGTGGTCAG (SEQ ID NO: 173) | TCGTCGGCAGCGTCAGATGTGTATAAGAGACAGNNNNNNNNGTAATGTGGTCAGNNNNNN (SEQ ID NO: 174) |
| P1_87 | BacDrop_RT10X_384_plate1 | H03 | GGCGCGCGAGCTG (SEQ ID NO: 175) | TCGTCGGCAGCGTCAGATGTGTATAAGAGACAGNNNNNNNNGGCGCGCGAGCTGNNNNNN (SEQ ID NO: 176) |
| P1_88 | BacDrop_RT10X_384_plate1 | H04 | GTCCATGTGTGTG (SEQ ID NO: 177) | TCGTCGGCAGCGTCAGATGTGTATAAGAGACAGNNNNNNNNGTCCATGTGTGTGNNNNNN (SEQ ID NO: 178) |
| P1_89 | BacDrop_RT10X_384_plate1 | H05 | GGGAGGAATCCAG (SEQ ID NO: 179) | TCGTCGGCAGCGTCAGATGTGTATAAGAGACAGNNNNNNNNGGGAGGAATCCAGNNNNNN (SEQ ID NO: 180) |
| P1_90 | BacDrop_RT10X_384_plate1 | H06 | GATCAAAATAGGG (SEQ ID NO: 181) | TCGTCGGCAGCGTCAGATGTGTATAAGAGACAGNNNNNNNNGATCAAAATAGGGNNNNNN (SEQ ID NO: 182) |
| P1_91 | BacDrop_RT10X_384_plate1 | H07 | GCAAGACTGCCAG (SEQ ID NO: 183) | TCGTCGGCAGCGTCAGATGTGTATAAGAGACAGNNNNNNNNGCAAGACTGCCAGNNNNNN (SEQ ID NO: 184) |
| P1_92 | BacDrop_RT10X_384_plate1 | H08 | GTAGGATGGACCG (SEQ ID NO: 185) | TCGTCGGCAGCGTCAGATGTGTATAAGAGACAGNNNNNNNNGTAGGATGGACCGNNNNNN (SEQ ID NO: 186) |
| P1_93 | BacDrop_RT10X_384_plate1 | H09 | GGATCGGACCTAG (SEQ ID NO: 187) | TCGTCGGCAGCGTCAGATGTGTATAAGAGACAGNNNNNNNNGGATCGGACCTAGNNNNNN (SEQ ID NO: 188) |
| P1_94 | BacDrop_RT10X_384_plate1 | H10 | GCCCAAAGGAATG (SEQ ID NO: 189) | TCGTCGGCAGCGTCAGATGTGTATAAGAGACAGNNNNNNNNGCCCAAAGGAATGNNNNNN (SEQ ID NO: 190) |
| P1_95 | BacDrop_RT10X_384_plate1 | H11 | GTACCTAACGCAG (SEQ ID NO: 191) | TCGTCGGCAGCGTCAGATGTGTATAAGAGACAGNNNNNNNNGTACCTAACGCAGNNNNNN (SEQ ID NO: 192) |
| P1_96 | BacDrop_RT10X_384_plate1 | H12 | GGTCAGGTTCAGG (SEQ ID NO: 193) | TCGTCGGCAGCGTCAGATGTGTATAAGAGACAGNNNNNNNNGGTCAGGTTCAGGNNNNNN (SEQ ID NO: 194) |
| P2_01 | BacDrop_RT10X_384_plate2 | A01 | AACCTCCAACCCA (SEQ ID NO: 195) | TCGTCGGCAGCGTCAGATGTGTATAAGAGACAGNNNNNNNNAACCTCCAACCCANNNNNN (SEQ ID NO: 196) |
| P2_02 | BacDrop_RT10X_384_plate2 | A02 | ACGTTTCAGCCAA (SEQ ID NO: 197) | TCGTCGGCAGCGTCAGATGTGTATAAGAGACAGNNNNNNNNACGTTTCAGCCAANNNNNN (SEQ ID NO: 198) |
| P2_03 | BacDrop_RT10X_384_plate2 | A03 | ATAAGGTTAGATA (SEQ ID NO: 199) | TCGTCGGCAGCGTCAGATGTGTATAAGAGACAGNNNNNNNNATAAGGTTAGATANNNNNN (SEQ ID NO: 200) |
| P2_04 | BacDrop_RT10X_384_plate2 | A04 | AATATACCAGACA (SEQ ID NO: 201) | TCGTCGGCAGCGTCAGATGTGTATAAGAGACAGNNNNNNNNAATATACCAGACANNNNNN (SEQ ID NO: 202) |
| P2_05 | BacDrop_RT10X_384_plate2 | A05 | AGGAGAGACTGTA (SEQ ID NO: 203) | TCGTCGGCAGCGTCAGATGTGTATAAGAGACAGNNNNNNNNAGGAGAGACTGTANNNNNN (SEQ ID NO: 204) |
| P2_06 | BacDrop_RT10X_384_plate2 | A06 | ACTCATCTTAGCA (SEQ ID NO: 205) | TCGTCGGCAGCGTCAGATGTGTATAAGAGACAGNNNNNNNNACTCATCTTAGCANNNNNN (SEQ ID NO: 206) |
| P2_07 | BacDrop_RT10X_384_plate2 | A07 | AAATTTGGCCGTA (SEQ ID NO: 207) | TCGTCGGCAGCGTCAGATGTGTATAAGAGACAGNNNNNNNNAAATTTGGCCGTANNNNNN (SEQ ID NO: 208) |
| P2_08 | BacDrop_RT10X_384_plate2 | A08 | ATTAAGGAAGTAA (SEQ ID NO: 209) | TCGTCGGCAGCGTCAGATGTGTATAAGAGACAGNNNNNNNNATTAAGGAAGTAANNNNNN (SEQ ID NO: 210) |
| P2_09 | BacDrop_RT10X_384_plate2 | A09 | AACCGATCCAGAA (SEQ ID NO: 211) | TCGTCGGCAGCGTCAGATGTGTATAAGAGACAGNNNNNNNNAACCGATCCAGAANNNNNN (SEQ ID NO: 212) |
| P2_10 | BacDrop_RT10X_384_plate2 | A10 | ACAACGGGACTGA (SEQ ID NO: 213) | TCGTCGGCAGCGTCAGATGTGTATAAGAGACAGNNNNNNNNACAACGGGACTGANNNNNN (SEQ ID NO: 214) |
| P2_11 | BacDrop_RT10X_384_plate2 | A11 | ATCCAGTACAACA (SEQ ID NO: 215) | TCGTCGGCAGCGTCAGATGTGTATAAGAGACAGNNNNNNNNATCCAGTACAACANNNNNN (SEQ ID NO: 216) |
| P2_12 | BacDrop_RT10X_384_plate2 | A12 | AGAGGACGATGCA (SEQ ID NO: 217) | TCGTCGGCAGCGTCAGATGTGTATAAGAGACAGNNNNNNNNAGAGGACGATGCANNNNNN (SEQ ID NO: 218) |
| P2_13 | BacDrop_RT10X_384_plate2 | B01 | AAAGGGTCGCTTA (SEQ ID NO: 219) | TCGTCGGCAGCGTCAGATGTGTATAAGAGACAGNNNNNNNNAAAGGGTCGCTTANNNNNN (SEQ ID NO: 220) |
| P2_14 | BacDrop_RT10X_384_plate2 | B02 | ACCCATGTAGCAA (SEQ ID NO: 221) | TCGTCGGCAGCGTCAGATGTGTATAAGAGACAGNNNNNNNNACCCATGTAGCAANNNNNN (SEQ ID NO: 222) |
| P2_15 | BacDrop_RT10X_384_plate2 | B03 | AGTGAAGTGGACA (SEQ ID NO: 223) | TCGTCGGCAGCGTCAGATGTGTATAAGAGACAGNNNNNNNNAGTGAAGTGGACANNNNNN (SEQ ID NO: 224) |
| P2_16 | BacDrop_RT10X_384_plate2 | B04 | ATAATAATTACAA (SEQ ID NO: 225) | TCGTCGGCAGCGTCAGATGTGTATAAGAGACAGNNNNNNNNATAATAATTACAANNNNNN (SEQ ID NO: 226) |
| P2_17 | BacDrop_RT10X_384_plate2 | B05 | AGCTGTAACGTGA (SEQ ID NO: 227) | TCGTCGGCAGCGTCAGATGTGTATAAGAGACAGNNNNNNNNAGCTGTAACGTGANNNNNN (SEQ ID NO: 228) |
| P2_18 | BacDrop_RT10X_384_plate2 | B06 | AAAAAGCTGCGTA (SEQ ID NO: 229) | TCGTCGGCAGCGTCAGATGTGTATAAGAGACAGNNNNNNNNAAAAAGCTGCGTANNNNNN (SEQ ID NO: 230) |
| P2_19 | BacDrop_RT10X_384_plate2 | B07 | ACGCGCAAACAGA (SEQ ID NO: 231) | TCGTCGGCAGCGTCAGATGTGTATAAGAGACAGNNNNNNNNACGCGCAAACAGANNNNNN (SEQ ID NO: 232) |
| P2_20 | BacDrop_RT10X_384_plate2 | B08 | ATAATTTTCCCCA (SEQ ID NO: 233) | TCGTCGG CAGCGTCAGATGTGTATAAGAGACAGNNNNNNNNATAATTTTCCCCANNNNNN (SEQ ID NO: 234) |
| P2_21 | BacDrop_RT10X_384_plate2 | B09 | AGGATACTGGTTA (SEQ ID NO: 235) | TCGTCGGCAGCGTCAGATGTGTATAAGAGACAGNNNNNNNNAGGATACTGGTTANNNNNN (SEQ ID NO: 236) |
| P2_22 | BacDrop_RT10X_384_plate2 | B10 | AAGGGGGAAGCGA (SEQ ID NO: 237) | TCGTCGGCAGCGTCAGATGTGTATAAGAGACAGNNNNNNNNAAGGGGGAAGCGANNNNNN (SEQ ID NO: 238) |
| P2_23 | BacDrop_RT10X_384_plate2 | B11 | ACAACGCCAAAAA (SEQ ID NO: 239) | TCGTCGGCAGCGTCAGATGTGTATAAGAGACAGNNNNNNNNACAACGCCAAAAANNNNNN (SEQ ID NO: 240) |
| P2_24 | BacDrop_RT10X_384_plate2 | B12 | AACGAAGCCTGTA (SEQ ID NO: 241) | TCGTCGGCAGCGTCAGATGTGTATAAGAGACAGNNNNNNNNAACGAAGCCTGTANNNNNN (SEQ ID NO: 242) |
| P2_25 | BacDrop_RT10X_384_plate2 | C01 | TTACGTTGCCAGA (SEQ ID NO: 243) | TCGTCGGCAGCGTCAGATGTGTATAAGAGACAGNNNNNNNNTTACGTTGCCAGANNNNNN (SEQ ID NO: 244) |
| P2_26 | BacDrop_RT10X_384_plate2 | C02 | TAATTAACTTATA (SEQ ID NO: 245) | TCGTCGGCAGCGTCAGATGTGTATAAGAGACAGNNNNNNNNTAATTAACTTATANNNNNN (SEQ ID NO: 246) |
| P2_27 | BacDrop_RT10X_384_plate2 | C03 | TCTCGGTATGGGA (SEQ ID NO: 247) | TCGTCGGCAGCGTCAGATGTGTATAAGAGACAGNNNNNNNNTCTCGGTATGGGANNNNNN (SEQ ID NO: 248) |
| P2_28 | BacDrop_RT10X_384_plate2 | C04 | TTCGTGTGAATTA (SEQ ID NO: 249) | TCGTCGGCAGCGTCAGATGTGTATAAGAGACAGNNNNNNNNTTCGTGTGAATTANNNNNN (SEQ ID NO: 250) |
| P2_29 | BacDrop_RT10X_384_plate2 | C05 | TATAATTAGTTCA (SEQ ID NO: 251) | TCGTCGGCAGCGTCAGATGTGTATAAGAGACAGNNNNNNNNTATAATTAGTTCANNNNNN (SEQ ID NO: 252) |
| P2_30 | BacDrop_RT10X_384_plate2 | C06 | TGGCTAATTCCTA (SEQ ID NO: 253) | TCGTCGGCAGCGTCAGATGTGTATAAGAGACAGNNNNNNNNTGGCTAATTCCTANNNNNN (SEQ ID NO: 254) |
| P2_31 | BacDrop_RT10X_384_plate2 | C07 | TAGAAGGTCGTGA (SEQ ID NO: 255) | TCGTCGGCAGCGTCAGATGTGTATAAGAGACAGNNNNNNNNTAGAAGGTCGTGANNNNNN (SEQ ID NO: 256) |
| P2_32 | BacDrop_RT10X_384_plate2 | C08 | TCGTGAGGGGGTA (SEQ ID NO: 257) | TCGTCGGCAGCGTCAGATGTGTATAAGAGACAGNNNNNNNNTCGTGAGGGGGTANNNNNN (SEQ ID NO: 258) |
| P2_33 | BacDrop_RT10X_384_plate2 | C09 | TTAGGCTAAAGGC (SEQ ID NO: 259) | TCGTCGGCAGCGTCAGATGTGTATAAGAGACAGNNNNNNNNTTAGGCTAAAGGCNNNNNN (SEQ ID NO: 260) |
| P2_34 | BacDrop_RT10X_384_plate2 | C10 | TGAAGTGGCTTCC (SEQ ID NO: 261) | TCGTCGGCAGCGTCAGATGTGTATAAGAGACAGNNNNNNNNTGAAGTGGCTTCCNNNNNN (SEQ ID NO: 262) |
| P2_35 | BacDrop_RT10X_384_plate2 | C11 | TATCGCATAGCGC (SEQ ID NO: 263) | TCGTCGGCAGCGTCAGATGTGTATAAGAGACAGNNNNNNNNTATCGCATAGCGCNNNNNN (SEQ ID NO: 264) |
| P2_36 | BacDrop_RT10X_384_plate2 | C12 | TCTCCTAAGCTCC (SEQ ID NO: 265) | TCGTCGGCAGCGTCAGATGTGTATAAGAGACAGNNNNNNNNTCTCCTAAGCTCCNNNNNN (SEQ ID NO: 266) |
| P2_37 | BacDrop_RT10X_384_plate2 | D01 | TAAATCATGATTC (SEQ ID NO: 267) | TCGTCGGCAGCGTCAGATGTGTATAAGAGACAGNNNNNNNNTAAATCATGATTCNNNNNN (SEQ ID NO: 268) |
| P2_38 | BacDrop_RT10X_384_plate2 | D02 | TCGCATACGGAAC (SEQ ID NO: 269) | TCGTCGGCAGCGTCAGATGTGTATAAGAGACAGNNNNNNNNTCGCATACGGAACNNNNNN (SEQ ID NO: 270) |
| P2_39 | BacDrop_RT10X_384_plate2 | D03 | TTGCAGCCACAGC (SEQ ID NO: 271) | TCGTCGGCAGCGTCAGATGTGTATAAGAGACAGNNNNNNNNTTGCAGCCACAGCNNNNNN (SEQ ID NO: 272) |
| P2_40 | BacDrop_RT10X_384_plate2 | D04 | TACCTGGTGCTTC (SEQ ID NO: 273) | TCGTCGGCAGCGTCAGATGTGTATAAGAGACAGNNNNNNNNTACCTGGTGCTTCNNNNNN (SEQ ID NO: 274) |
| P2_41 | BacDrop_RT10X_384_plate2 | D05 | TGTTTTCATAAGC (SEQ ID NO: 275) | TCGTCGGCAGCGTCAGATGTGTATAAGAGACAGNNNNNNNNTGTTTTCATAAGCNNNNNN (SEQ ID NO: 276) |
| P2_42 | BacDrop_RT10X_384_plate2 | D06 | TAAACTGAGATGC (SEQ ID NO: 277) | TCGTCGGCAGCGTCAGATGTGTATAAGAGACAGNNNNNNNNTAAACTGAGATGCNNNNNN (SEQ ID NO: 278) |
| P2_43 | BacDrop_RT10X_384_plate2 | D07 | TCAGTGCGCTGTC (SEQ ID NO: 279) | TCGTCGGCAGCGTCAGATGTGTATAAGAGACAGNNNNNNNNTCAGTGCGCTGTCNNNNNN (SEQ ID NO: 280) |
| P2_44 | BacDrop_RT10X_384_plate2 | D08 | TTGAGTGGGCCAC (SEQ ID NO: 281) | TCGTCGGCAGCGTCAGATGTGTATAAGAGACAGNNNNNNNNTTGAGTGGGCCACNNNNNN (SEQ ID NO: 282) |
| P2_45 | BacDrop_RT10X_384_plate2 | D09 | TATTCTCTCAACC (SEQ ID NO: 283) | TCGTCGG CAGCGTCAGATGTGTATAAGAGACAGNNNNNNNNTATTCTCTCAACCNNNNNN (SEQ ID NO: 284) |
| P2_46 | BacDrop_RT10X_384_plate2 | D10 | TCCTCCTTTACGC (SEQ ID NO: 285) | TCGTCGGCAGCGTCAGATGTGTATAAGAGACAGNNNNNNNNTCCTCCTTTACGCNNNNNN (SEQ ID NO: 286) |
| P2_47 | BacDrop_RT10X_384_plate2 | D11 | TAGAGAACTTTAC (SEQ ID NO: 287) | TCGTCGGCAGCGTCAGATGTGTATAAGAGACAGNNNNNNNNTAGAGAACTTTACNNNNNN (SEQ ID NO: 288) |
| P2_48 | BacDrop_RT10X_384_plate2 | D12 | TTACTACCAGGTC (SEQ ID NO: 289) | TCGTCGGCAGCGTCAGATGTGTATAAGAGACAGNNNNNNNNTTACTACCAGGTCNNNNNN (SEQ ID NO: 290) |
| P2_49 | BacDrop_RT10X_384_plate2 | E01 | CATTGCTACATCC (SEQ ID NO: 291) | TCGTCGGCAGCGTCAGATGTGTATAAGAGACAGNNNNNNNNCATTGCTACATCCNNNNNN (SEQ ID NO: 292) |
| P2_50 | BacDrop_RT10X_384_plate2 | E02 | CGGAGTCACACGC (SEQ ID NO: 293) | TCGTCGGCAGCGTCAGATGTGTATAAGAGACAGNNNNNNNNCGGAGTCACACGCNNNNNN (SEQ ID NO: 294) |
| P2_51 | BacDrop_RT10X_384_plate2 | E03 | CCGGTGGAGCTCC (SEQ ID NO: 295) | TCGTCGGCAGCGTCAGATGTGTATAAGAGACAGNNNNNNNNCCGGTGGAGCTCCNNNNNN (SEQ ID NO: 296) |
| P2_52 | BacDrop_RT10X_384_plate2 | E04 | CTTCGGCCCAATC (SEQ ID NO: 297) | TCGTCGGCAGCGTCAGATGTGTATAAGAGACAGNNNNNNNNCTTCGGCCCAATCNNNNNN (SEQ ID NO: 298) |
| P2_53 | BacDrop_RT10X_384_plate2 | E05 | CACGAGACACCCC (SEQ ID NO: 299) | TCGTCGGCAGCGTCAGATGTGTATAAGAGACAGNNNNNNNNCACGAGACACCCCNNNNNN (SEQ ID NO: 300) |
| P2_54 | BacDrop_RT10X_384_plate2 | E06 | CGTCTAGACAAAC (SEQ ID NO: 301) | TCGTCGGCAGCGTCAGATGTGTATAAGAGACAGNNNNNNNNCGTCTAGACAAACNNNNNN (SEQ ID NO: 302) |
| P2_55 | BacDrop_RT10X_384_plate2 | E07 | CACCGTACGTTGC (SEQ ID NO: 303) | TCGTCGGCAGCGTCAGATGTGTATAAGAGACAGNNNNNNNNCACCGTACGTTGCNNNNNN (SEQ ID NO: 304) |
| P2_56 | BacDrop_RT10X_384_plate2 | E08 | CCTAACCCTCTCC (SEQ ID NO: 305) | TCGTCGGCAGCGTCAGATGTGTATAAGAGACAGNNNNNNNNCCTAACCCTCTCCNNNNNN (SEQ ID NO: 306) |
| P2_57 | BacDrop_RT10X_384_plate2 | E09 | CTAGATTTAACTC (SEQ ID NO: 307) | TCGTCGGCAGCGTCAGATGTGTATAAGAGACAGNNNNNNNNCTAGATTTAACTCNNNNNN (SEQ ID NO: 308) |
| P2_58 | BacDrop_RT10X_384_plate2 | E10 | CAGATTAGAGTAC (SEQ ID NO: 309) | TCGTCGGCAGCGTCAGATGTGTATAAGAGACAGNNNNNNNNCAGATTAGAGTACNNNNNN (SEQ ID NO: 310) |
| P2_59 | BacDrop_RT10X_384_plate2 | E11 | CGGGACGTTTTCC (SEQ ID NO: 311) | TCGTCGGCAGCGTCAGATGTGTATAAGAGACAGNNNNNNNNCGGGACGTTTTCCNNNNNN (SEQ ID NO: 312) |
| P2_60 | BacDrop_RT10X_384_plate2 | E12 | CTATACCAAGAAC (SEQ ID NO: 313) | TCGTCGGCAGCGTCAGATGTGTATAAGAGACAGNNNNNNNNCTATACCAAGAACNNNNNN (SEQ ID NO: 314) |
| P2_61 | BacDrop_RT10X_384_plate2 | F01 | CCGGCGAGCCAGC (SEQ ID NO: 315) | TCGTCGGCAGCGTCAGATGTGTATAAGAGACAGNNNNNNNNCCGGCGAGCCAGCNNNNNN (SEQ ID NO: 316) |
| P2_62 | BacDrop_RT10X_384_plate2 | F02 | CATTTGAAAACGC (SEQ ID NO: 317) | TCGTCGGCAGCGTCAGATGTGTATAAGAGACAGNNNNNNNNCATTTGAAAACGCNNNNNN (SEQ ID NO: 318) |
| P2_63 | BacDrop_RT10X_384_plate2 | F03 | CCCCTTTCTGGAC (SEQ ID NO: 319) | TCGTCGGCAGCGTCAGATGTGTATAAGAGACAGNNNNNNNNCCCCTTTCTGGACNNNNNN (SEQ ID NO: 320) |
| P2_64 | BacDrop_RT10X_384_plate2 | F04 | CTGACTGCAGCGC (SEQ ID NO: 321) | TCGTCGGCAGCGTCAGATGTGTATAAGAGACAGNNNNNNNNCTGACTGCAGCGCNNNNNN (SEQ ID NO: 322) |
| P2_65 | BacDrop_RT10X_384_plate2 | F05 | CACGATCAAGTTG (SEQ ID NO: 323) | TCGTCGGCAGCGTCAGATGTGTATAAGAGACAGNNNNNNNNCACGATCAAGTTGNNNNNN (SEQ ID NO: 324) |
| P2_66 | BacDrop_RT10X_384_plate2 | F06 | CGAAATGCTGACG (SEQ ID NO: 325) | TCGTCGGCAGCGTCAGATGTGTATAAGAGACAGNNNNNNNNCGAAATGCTGACGNNNNNN (SEQ ID NO: 326) |
| P2_67 | BacDrop_RT10X_384_plate2 | F07 | CGCTACCTGAAGG (SEQ ID NO: 327) | TCGTCGGCAGCGTCAGATGTGTATAAGAGACAGNNNNNNNNCGCTACCTGAAGGNNNNNN (SEQ ID NO: 328) |
| P2_68 | BacDrop_RT10X_384_plate2 | F08 | CAGGAACTACACG (SEQ ID NO: 329) | TCGTCGGCAGCGTCAGATGTGTATAAGAGACAGNNNNNNNNCAGGAACTACACGNNNNNN (SEQ ID NO: 330) |
| P2_69 | BacDrop_RT10X_384_plate2 | F09 | CTCGGCCGGTCAG (SEQ ID NO: 331) | TCGTCGGCAGCGTCAGATGTGTATAAGAGACAGNNNNNNNNCTCGGCCGGTCAGNNNNNN (SEQ ID NO: 332) |
| P2_70 | BacDrop_RT10X_384_plate2 | F10 | CTTTTCCAGCGTG (SEQ ID NO: 333) | TCGTCGGCAGCGTCAGATGTGTATAAGAGACAGNNNNNNNNCTTTTCCAGCGTGNNNNNN (SEQ ID NO: 334) |
| P2_71 | BacDrop_RT10X_384_plate2 | F11 | CAATCGTAGCCGG (SEQ ID NO: 335) | TCGTCGGCAGCGTCAGATGTGTATAAGAGACAGNNNNNNNNCAATCGTAGCCGGNNNNNN (SEQ ID NO: 336) |
| P2_72 | BacDrop_RT10X_384_plate2 | F12 | CGTAAACTACGAG (SEQ ID NO: 337) | TCGTCGGCAGCGTCAGATGTGTATAAGAGACAGNNNNNNNNCGTAAACTACGAGNNNNNN (SEQ ID NO: 338) |
| P2_73 | BacDrop_RT10X_384_plate2 | G01 | GACGAGTATCTGG (SEQ ID NO: 339) | TCGTCGGCAGCGTCAGATGTGTATAAGAGACAGNNNNNNNNGACGAGTATCTGGNNNNNN (SEQ ID NO: 340) |
| P2_74 | BacDrop_RT10X_384_plate2 | G02 | GCAAAGGGTGGTG (SEQ ID NO: 341) | TCGTCGGCAGCGTCAGATGTGTATAAGAGACAGNNNNNNNNGCAAAGGGTGGTGNNNNNN (SEQ ID NO: 342) |
| P2_75 | BacDrop_RT10X_384_plate2 | G03 | GTAGGTGAGAAAG (SEQ ID NO: 343) | TCGTCGGCAGCGTCAGATGTGTATAAGAGACAGNNNNNNNNGTAGGTGAGAAAGNNNNNN (SEQ ID NO: 344) |
| P2_76 | BacDrop_RT10X_384_plate2 | G04 | GAGCGGATTAGTG (SEQ ID NO: 345) | TCGTCGGCAGCGTCAGATGTGTATAAGAGACAGNNNNNNNNGAGCGGATTAGTGNNNNNN (SEQ ID NO: 346) |
| P2_77 | BacDrop_RT10X_384_plate2 | G05 | GGTTTGTATGTCG (SEQ ID NO: 347) | TCGTCGGCAGCGTCAGATGTGTATAAGAGACAGNNNNNNNNGGTTTGTATGTCGNNNNNN (SEQ ID NO: 348) |
| P2_78 | BacDrop_RT10X_384_plate2 | G06 | GAACACATCTGAG (SEQ ID NO: 349) | TCGTCGGCAGCGTCAGATGTGTATAAGAGACAGNNNNNNNNGAACACATCTGAGNNNNNN (SEQ ID NO: 350) |
| P2_79 | BacDrop_RT10X_384_plate2 | G07 | GCTACCAATGACG (SEQ ID NO: 351) | TCGTCGGCAGCGTCAGATGTGTATAAGAGACAGNNNNNNNNGCTACCAATGACGNNNNNN (SEQ ID NO: 352) |
| P2_80 | BacDrop_RT10X_384_plate2 | G08 | GTGAGGGCTCGGG (SEQ ID NO: 353) | TCGTCGGCAGCGTCAGATGTGTATAAGAGACAGNNNNNNNNGTGAGGGCTCGGGNNNNNN (SEQ ID NO: 354) |
| P2_81 | BacDrop_RT10X_384_plate2 | G09 | GAAATGTGTCGCG (SEQ ID NO: 355) | TCGTCGGCAGCGTCAGATGTGTATAAGAGACAGNNNNNNNNGAAATGTGTCGCGNNNNNN (SEQ ID NO: 356) |
| P2_82 | BacDrop_RT10X_384_plate2 | G10 | GGCCCACACCGGG (SEQ ID NO: 357) | TCGTCGGCAGCGTCAGATGTGTATAAGAGACAGNNNNNNNNGGCCCACACCGGGNNNNNN (SEQ ID NO: 358) |
| P2_83 | BacDrop_RT10X_384_plate2 | G11 | GCCGGTAGCTCCG (SEQ ID NO: 359) | TCGTCGGCAGCGTCAGATGTGTATAAGAGACAGNNNNNNNNGCCGGTAGCTCCGNNNNNN (SEQ ID NO: 360) |
| P2_84 | BacDrop_RT10X_384_plate2 | G12 | GAAGAATTATGGG (SEQ ID NO: 361) | TCGTCGGCAGCGTCAGATGTGTATAAGAGACAGNNNNNNNNGAAGAATTATGGGNNNNNN (SEQ ID NO: 362) |
| P2_85 | BacDrop_RT10X_384_plate2 | H01 | GTCTCGTCATATG (SEQ ID NO: 363) | TCGTCGGCAGCGTCAGATGTGTATAAGAGACAGNNNNNNNNGTCTCGTCATATGNNNNNN (SEQ ID NO: 364) |
| P2_86 | BacDrop_RT10X_384_plate2 | H02 | GGACTTTTGGTCG (SEQ ID NO: 365) | TCGTCGGCAGCGTCAGATGTGTATAAGAGACAGNNNNNNNNGGACTTTTGGTCGNNNNNN (SEQ ID NO: 366) |
| P2_87 | BacDrop_RT10X_384_plate2 | H03 | GATTTCGTTCCAG (SEQ ID NO: 367) | TCGTCGGCAGCGTCAGATGTGTATAAGAGACAGNNNNNNNNGATTTCGTTCCAGNNNNNN (SEQ ID NO: 368) |
| P2_88 | BacDrop_RT10X_384_plate2 | H04 | GTCCCGGAGCCAG (SEQ ID NO: 369) | TCGTCGGCAGCGTCAGATGTGTATAAGAGACAGNNNNNNNNGTCCCGGAGCCAGNNNNNN (SEQ ID NO: 370) |
| P2_89 | BacDrop_RT10X_384_plate2 | H05 | GGGGGGCCGGACG (SEQ ID NO: 371) | TCGTCGGCAGCGTCAGATGTGTATAAGAGACAGNNNNNNNNGGGGGGCCGGACGNNNNNN (SEQ ID NO: 372) |
| P2_90 | BacDrop_RT10X_384_plate2 | H06 | GACTACGTTTGGG (SEQ ID NO: 373) | TCGTCGGCAGCGTCAGATGTGTATAAGAGACAGNNNNNNNNGACTACGTTTGGGNNNNNN (SEQ ID NO: 374) |
| P2_91 | BacDrop_RT10X_384_plate2 | H07 | GCTTTAATCTAAG (SEQ ID NO: 375) | TCGTCGGCAGCGTCAGATGTGTATAAGAGACAGNNNNNNNNGCTTTAATCTAAGNNNNNN (SEQ ID NO: 376) |
| P2_92 | BacDrop_RT10X_384_plate2 | H08 | GAGGCAAAAATCG (SEQ ID NO: 377) | TCGTCGGCAGCGTCAGATGTGTATAAGAGACAGNNNNNNNNGAGGCAAAAATCGNNNNNN (SEQ ID NO: 378) |
| P2_93 | BacDrop_RT10X_384_plate2 | H09 | GTTTGATTCTCGG (SEQ ID NO: 379) | TCGTCGGCAGCGTCAGATGTGTATAAGAGACAGNNNNNNNNGTTTGATTCTCGGNNNNNN (SEQ ID NO: 380) |
| P2_94 | BacDrop_RT10X_384_plate2 | H10 | GAGGGACATCGAG (SEQ ID NO: 381) | TCGTCGGCAGCGTCAGATGTGTATAAGAGACAGNNNNNNNNGAGGGACATCGAGNNNNNN (SEQ ID NO: 382) |
| P2_95 | BacDrop_RT10X_384_plate2 | H11 | GGATCCTTACCCG (SEQ ID NO: 383) | TCGTCGG CAGCGTCAGATGTGTATAAGAGACAG NNNNNNNNGGATCCTTACCCG NNNNNN (SEQ ID NO: 384) |
| P2_96 | BacDrop_RT10X_384_plate2 | H12 | GCGATCCTCGCGG (SEQ ID NO: 385) | TCGTCGGCAGCGTCAGATGTGTATAAGAGACAGNNNNNNNNGCGATCCTCGCGGNNNNNN (SEQ ID NO: 386) |
| P3_01 | BacDrop_RT10X_384_plate3 | A01 | ATATTGGCACTTA (SEQ ID NO: 387) | TCGTCGGCAGCGTCAGATGTGTATAAGAGACAGNNNNNNNNATATTGGCACTTANNNNNN (SEQ ID NO: 388) |
| P3_02 | BacDrop_RT10X_384_plate3 | A02 | AAAAGAATCGCCA (SEQ ID NO: 389) | TCGTCGGCAGCGTCAGATGTGTATAAGAGACAGNNNNNNNNAAAAGAATCGCCANNNNNN (SEQ ID NO: 390) |
| P3_03 | BacDrop_RT10X_384_plate3 | A03 | AGGAAGCCCATTA (SEQ ID NO: 391) | TCGTCGGCAGCGTCAGATGTGTATAAGAGACAGNNNNNNNNAGGAAGCCCATTANNNNNN (SEQ ID NO: 392) |
| P3_04 | BacDrop_RT10X_384_plate3 | A04 | ACCCCTTCGCAGA (SEQ ID NO: 393) | TCGTCGGCAGCGTCAGATGTGTATAAGAGACAGNNNNNNNNACCCCTTCGCAGANNNNNN (SEQ ID NO: 394) |
| P3_05 | BacDrop_RT10X_384_plate3 | A05 | ATGTCTATTCACA (SEQ ID NO: 395) | TCGTCGGCAGCGTCAGATGTGTATAAGAGACAGNNNNNNNNATGTCTATTCACANNNNNN (SEQ ID NO: 396) |
| P3_06 | BacDrop_RT10X_384_plate3 | A06 | AGAACGCTTTGAA (SEQ ID NO: 397) | TCGTCGGCAGCGTCAGATGTGTATAAGAGACAGNNNNNNNNAGAACGCTTTGAANNNNNN (SEQ ID NO: 398) |
| P3_07 | BacDrop_RT10X_384_plate3 | A07 | AAGGGACCCACCA (SEQ ID NO: 399) | TCGTCGGCAGCGTCAGATGTGTATAAGAGACAGNNNNNNNNAAGGGACCCACCANNNNNN (SEQ ID NO: 400) |
| P3_08 | BacDrop_RT10X_384_plate3 | A08 | ACACATCGTTTTA (SEQ ID NO: 401) | TCGTCGGCAGCGTCAGATGTGTATAAGAGACAGNNNNNNNNACACATCGTTTTANNNNNN (SEQ ID NO: 402) |
| P3_09 | BacDrop_RT10X_384_plate3 | A09 | AATGGTTCTCCGA (SEQ ID NO: 403) | TCGTCGGCAGCGTCAGATGTGTATAAGAGACAGNNNNNNNNAATGGTTCTCCGANNNNNN (SEQ ID NO: 404) |
| P3_10 | BacDrop_RT10X_384_plate3 | A10 | ATGCCGCTGAAAA (SEQ ID NO: 405) | TCGTCGGCAGCGTCAGATGTGTATAAGAGACAGNNNNNNNNATGCCGCTGAAAANNNNNN (SEQ ID NO: 406) |
| P3_11 | BacDrop_RT10X_384_plate3 | A11 | AAACACTCTAGTA (SEQ ID NO: 407) | TCGTCGGCAGCGTCAGATGTGTATAAGAGACAGNNNNNNNNAAACACTCTAGTANNNNNN (SEQ ID NO: 408) |
| P3_12 | BacDrop_RT10X_384_plate3 | A12 | ATCCTCGGGGTCA (SEQ ID NO: 409) | TCGTCGGCAGCGTCAGATGTGTATAAGAGACAGNNNNNNNNATCCTCGGGGTCANNNNNN (SEQ ID NO: 410) |
| P3_13 | BacDrop_RT10X_384_plate3 | B01 | AAGAATCGCTAGA (SEQ ID NO: 411) | TCGTCGGCAGCGTCAGATGTGTATAAGAGACAGNNNNNNNNAAGAATCGCTAGANNNNNN (SEQ ID NO: 412) |
| P3_14 | BacDrop_RT10X_384_plate3 | B02 | AGAAGTCAGGTAA (SEQ ID NO: 413) | TCGTCGGCAGCGTCAGATGTGTATAAGAGACAGNNNNNNNNAGAAGTCAGGTAANNNNNN (SEQ ID NO: 414) |
| P3_15 | BacDrop_RT10X_384_plate3 | B03 | ACGCGGCTCATCA (SEQ ID NO: 415) | TCGTCGGCAGCGTCAGATGTGTATAAGAGACAGNNNNNNNNACGCGGCTCATCANNNNNN (SEQ ID NO: 416) |
| P3_16 | BacDrop _RT10X _384 _plate3 | B04 | AAACCCAAATATA (SEQ ID NO: 417) | TCGTCGGCAGCGTCAGATGTGTATAAGAGACAGNNNNNNNNAAACCCAAATATANNNNNN (SEQ ID NO: 418) |
| P3_17 | BacDrop _RT10X _384 _plate3 | B05 | ATCTCAGAACTGA (SEQ ID NO: 419) | TCGTCGGCAGCGTCAGATGTGTATAAGAGACAGNNNNNNNNATCTCAGAACTGANNNNNN (SEQ ID NO: 420) |
| P3_18 | BacDrop_RT10X_384_plate3 | B06 | AGCAATTAGGAGA (SEQ ID NO: 421) | TCGTCGGCAGCGTCAGATGTGTATAAGAGACAGNNNNNNNNAGCAATTAGGAGANNNNNN (SEQ ID NO: 422) |
| P3_19 | BacDrop_RT10X _384 _plate3 | B07 | AAGTAAACGCGTA (SEQ ID NO: 423) | TCGTCGGCAGCGTCAGATGTGTATAAGAGACAGNNNNNNNNAAGTAAACGCGTANNNNNN (SEQ ID NO: 424) |
| P3_20 | BacDrop_RT10X _384 _plate3 | B08 | ACATACAGCATGA (SEQ ID NO: 425) | TCGTCGGCAGCGTCAGATGTGTATAAGAGACAGNNNNNNNNACATACAGCATGANNNNNN (SEQ ID NO: 426) |
| P3_21 | BacDrop_RT10X _384 _plate3 | B09 | AACGCAGAGAGAA (SEQ ID NO: 427) | TCGTCGGCAGCGTCAGATGTGTATAAGAGACAGNNNNNNNNAACGCAGAGAGAANNNNNN (SEQ ID NO: 428) |
| P3_22 | BacDrop_RT10X _384 _plate3 | B10 | ATGACGCGATTTA (SEQ ID NO: 429) | TCGTCGGCAGCGTCAGATGTGTATAAGAGACAGNNNNNNNNATGACGCGATTTANNNNNN (SEQ ID NO: 430) |
| P3_23 | BacDrop_RT10X_384_plate3 | B11 | AAACATTCACCAA (SEQ ID NO: 431) | TCGTCGGCAGCGTCAGATGTGTATAAGAGACAGNNNNNNNNAAACATTCACCAANNNNNN (SEQ ID NO: 432) |
| P3_24 | BacDrop _RT10X _384 _plate3 | B12 | AGGCCTCATATTA (SEQ ID NO: 433) | TCGTCGGCAGCGTCAGATGTGTATAAGAGACAGNNNNNNNNAGGCCTCATATTANNNNNN (SEQ ID NO: 434) |
| P3_25 | BacDrop _RT10X _384 _plate3 | C01 | TGACGCTCACTGA (SEQ ID NO: 435) | TCGTCGGCAGCGTCAGATGTGTATAAGAGACAGNNNNNNNNTGACGCTCACTGANNNNNN (SEQ ID NO: 436) |
| P3_26 | BacDrop _RT10X _384 _plate3 | C02 | TCCGGGCGGAGCA (SEQ ID NO: 437) | TCGTCGGCAGCGTCAGATGTGTATAAGAGACAGNNNNNNNNTCCGGGCGGAGCANNNNNN (SEQ ID NO: 438) |
| P3_27 | BacDrop _RT10X _384 _plate3 | C03 | TTTCCCGTTTAAA (SEQ ID NO: 439) | TCGTCGGCAGCGTCAGATGTGTATAAGAGACAGNNNNNNNNTTTCCCGTTTAAANNNNNN (SEQ ID NO: 440) |
| P3_28 | BacDrop_RT10X _384 _plate3 | C04 | TGTAACTGAGTGA (SEQ ID NO: 441) | TCGTCGGCAGCGTCAGATGTGTATAAGAGACAGNNNNNNNNTGTAACTGAGTGANNNNNN (SEQ ID NO: 442) |
| P3_29 | BacDrop_RT10X _384 _plate3 | C05 | TAACCTATGCCTA (SEQ ID NO: 443) | TCGTCGGCAGCGTCAGATGTGTATAAGAGACAGNNNNNNNNTAACCTATGCCTANNNNNN (SEQ ID NO: 444) |
| P3_30 | BacDrop_RT10X _384 _plate3 | C06 | TCCAAAGAACATA (SEQ ID NO: 445) | TCGTCGGCAGCGTCAGATGTGTATAAGAGACAGNNNNNNNNTCCAAAGAACATANNNNNN (SEQ ID NO: 446) |
| P3_31 | BacDrop_RT10X _384 _plate3 | C07 | TTCTCGGGTGACA (SEQ ID NO: 447) | TCGTCGGCAGCGTCAGATGTGTATAAGAGACAGNNNNNNNNTTCTCGGGTGACANNNNNN (SEQ ID NO: 448) |
| P3_32 | BacDrop_RT10X _384 _plate3 | C08 | TGGTGTTGTTTTA (SEQ ID NO: 449) | TCGTCGGCAGCGTCAGATGTGTATAAGAGACAGNNNNNNNNTGGTGTTGTTTTANNNNNN (SEQ ID NO: 450) |
| P3_33 | BacDrop _RT10X _384 _plate3 | C09 | TAACCCGTAGTAC (SEQ ID NO: 451) | TCGTCGGCAGCGTCAGATGTGTATAAGAGACAGNNNNNNNNTAACCCGTAGTACNNNNNN (SEQ ID NO: 452) |
| P3_34 | BacDrop _RT10X _384 _plate3 | C10 | TCTGCCTACTAAC (SEQ ID NO: 453) | TCGTCGGCAGCGTCAGATGTGTATAAGAGACAGNNNNNNNNTCTGCCTACTAACNNNNNN (SEQ ID NO: 454) |
| P3_35 | BacDrop _RT10X _384 _plate3 | C11 | TTCAGTATTTATC (SEQ ID NO: 455) | TCGTCGGCAGCGTCAGATGTGTATAAGAGACAGNNNNNNNNTTCAGTATTTATCNNNNNN (SEQ ID NO: 456) |
| P3_36 | BacDrop _RT10X _384 _plate3 | C12 | TGCTAGGGAAAAC (SEQ ID NO: 457) | TCGTCGGCAGCGTCAGATGTGTATAAGAGACAGNNNNNNNNTGCTAGGGAAAACNNNNNN (SEQ ID NO: 458) |
| P3_37 | BacDrop _RT10X _384 _plate3 | D01 | TAGTTACGAGGGC (SEQ ID NO: 459) | TCGTCGGCAGCGTCAGATGTGTATAAGAGACAGNNNNNNNNTAGTTACGAGGGCNNNNNN (SEQ ID NO: 460) |
| P3_38 | BacDrop_RT10X _384 _plate3 | D02 | TCGACCTAGGTTC (SEQ ID NO: 461) | TCGTCGGCAGCGTCAGATGTGTATAAGAGACAGNNNNNNNNTCGACCTAGGTTCNNNNNN (SEQ ID NO: 462) |
| P3_39 | BacDrop_RT10X _384 _plate3 | D03 | TGCCTCGATAGGC (SEQ ID NO: 463) | TCGTCGGCAGCGTCAGATGTGTATAAGAGACAGNNNNNNNNTGCCTCGATAGGCNNNNNN (SEQ ID NO: 464) |
| P3_40 | BacDrop_RT10X _384 _plate3 | D04 | TATTAGTCAAACC (SEQ ID NO: 465) | TCGTCGGCAGCGTCAGATGTGTATAAGAGACAGNNNNNNNNTATTAGTCAAACCNNNNNN (SEQ ID NO: 466) |
| P3_41 | BacDrop_RT10X _384 _plate3 | D05 | TCTTACTGAACTC (SEQ ID NO: 467) | TCGTCGGCAGCGTCAGATGTGTATAAGAGACAGNNNNNNNNTCTTACTGAACTCNNNNNN (SEQ ID NO: 468) |
| P3_42 | BacDrop_RT10X _384 _plate3 | D06 | TTCAATTGTGCAC (SEQ ID NO: 469) | TCGTCGGCAGCGTCAGATGTGTATAAGAGACAGNNNNNNNNTTCAATTGTGCACNNNNNN (SEQ ID NO: 470) |
| P3_43 | BacDrop _RT10X _384 _plate3 | D07 | TGTACATGTCACC (SEQ ID NO: 471) | TCGTCGGCAGCGTCAGATGTGTATAAGAGACAGNNNNNNNNTGTACATGTCACCNNNNNN (SEQ ID NO: 472) |
| P3_44 | BacDrop _RT10X _384 _plate3 | D08 | TACGTATCATTAC (SEQ ID NO: 473) | TCGTCGGCAGCGTCAGATGTGTATAAGAGACAGNNNNNNNNTACGTATCATTACNNNNNN (SEQ ID NO: 474) |
| P3_45 | BacDrop _RT10X _384 _plate3 | D09 | TCATGGACTCTGC (SEQ ID NO: 475) | TCGTCGGCAGCGTCAGATGTGTATAAGAGACAGNNNNNNNNTCATGGACTCTGCNNNNNN (SEQ ID NO: 476) |
| P3_46 | BacDrop _RT10X _384 _plate3 | D10 | TTCATGCTCCATC (SEQ ID NO: 477) | TCGTCGGCAGCGTCAGATGTGTATAAGAGACAGNNNNNNNNTTCATGCTCCATCNNNNNN (SEQ ID NO: 478) |
| P3_47 | BacDrop _RT10X _384 _plate3 | D11 | TGCTAACTCTGCC (SEQ ID NO: 479) | TCGTCGGCAGCGTCAGATGTGTATAAGAGACAGNNNNNNNNTGCTAACTCTGCCNNNNNN (SEQ ID NO: 480) |
| P3_48 | BacDrop_RT10X _384 _plate3 | D12 | TCCCTACTTCAAC (SEQ ID NO: 481) | TCGTCGGCAGCGTCAGATGTGTATAAGAGACAGNNNNNNNNTCCCTACTTCAACNNNNNN (SEQ ID NO: 482) |
| P3_49 | BacDrop_RT10X _384 _plate3 | E01 | CAGTACTACGCGC (SEQ ID NO: 483) | TCGTCGGCAGCGTCAGATGTGTATAAGAGACAGNNNNNNNNCAGTACTACGCGCNNNNNN (SEQ ID NO: 484) |
| P3_50 | BacDrop_RT10X _384 _plate3 | E02 | CTCACCGGCTATC (SEQ ID NO: 485) | TCGTCGGCAGCGTCAGATGTGTATAAGAGACAGNNNNNNNNCTCACCGGCTATCNNNNNN (SEQ ID NO: 486) |
| P3_51 | BacDrop_RT10X _384 _plate3 | E03 | CACTATTTAATAC (SEQ ID NO: 487) | TCGTCGGCAGCGTCAGATGTGTATAAGAGACAGNNNNNNNNCACTATTTAATACNNNNNN (SEQ ID NO: 488) |
| P3_52 | BacDrop_RT10X_384_plate3 | E04 | CCGGACTTGCTGC (SEQ ID NO: 489) | TCGTCGGCAGCGTCAGATGTGTATAAGAGACAGNNNNNNNNCCGGACTTGCTGCNNNNNN (SEQ ID NO: 490) |
| P3_53 | BacDrop _RT10X _384 _plate3 | E05 | CTCACATCTTCCC (SEQ ID NO: 491) | TCGTCGG CAGCGTCAGATGTGTATAAGAGACAGNNNNNNNNCTCACATCTTCCCNNNNNN (SEQ ID NO: 492) |
| P3_54 | BacDrop _RT10X _384 _plate3 | E06 | CGACCACCTGTAC (SEQ ID NO: 493) | TCGTCGGCAGCGTCAGATGTGTATAAGAGACAGNNNNNNNNCGACCACCTGTACNNNNNN (SEQ ID NO: 494) |
| P3_55 | BacDrop_RT10X_384_plate3 | E07 | CATCAATCCCAGC (SEQ ID NO: 495) | TCGTCGGCAGCGTCAGATGTGTATAAGAGACAGNNNNNNNNCATCAATCCCAGCNNNNNN (SEQ ID NO: 496) |
| P3_56 | BacDrop_RT10X _384 _plate3 | E08 | CCCAAGCAGGCCC (SEQ ID NO: 497) | TCGTCGGCAGCGTCAGATGTGTATAAGAGACAGNNNNNNNNCCCAAGCAGGCCCNNNNNN (SEQ ID NO: 498) |
| P3_57 | BacDrop_RT10X _384 _plate3 | E09 | CTGTGCATAGTTC (SEQ ID NO: 499) | TCGTCGGCAGCGTCAGATGTGTATAAGAGACAGNNNNNNNNCTGTGCATAGTTCNNNNNN (SEQ ID NO: 500) |
| P3_58 | BacDrop_RT10X _384 _plate3 | E10 | CAACGCAGTCCAC (SEQ ID NO: 501) | TCGTCGGCAGCGTCAGATGTGTATAAGAGACAGNNNNNNNNCAACGCAGTCCACNNNNNN (SEQ ID NO: 502) |
| P3_59 | BacDrop_RT10X _384 _plate3 | E11 | CGCGTTGGTAATC (SEQ ID NO: 503) | TCGTCGGCAGCGTCAGATGTGTATAAGAGACAGNNNNNNNNCGCGTTGGTAATCNNNNNN (SEQ ID NO: 504) |
| P3_60 | BacDrop_RT10X_384_plate3 | E12 | CCTTCAGTAGGAC (SEQ ID NO: 505) | TCGTCGGCAGCGTCAGATGTGTATAAGAGACAGNNNNNNNNCCTTCAGTAGGACNNNNNN (SEQ ID NO: 506) |
| P3_61 | BacDrop _RT10X _384 _plate3 | F01 | CTAGCCTTCTGTC (SEQ ID NO: 507) | TCGTCGGCAGCGTCAGATGTGTATAAGAGACAGNNNNNNNNCTAGCCTTCTGTCNNNNNN (SEQ ID NO: 508) |
| P3_62 | BacDrop _RT10X _384 _plate3 | F02 | CGTCCCACGAAGC (SEQ ID NO: 509) | TCGTCGGCAGCGTCAGATGTGTATAAGAGACAGNNNNNNNNCGTCCCACGAAGCNNNNNN (SEQ ID NO: 510) |
| P3_63 | BacDrop _RT10X _384 _plate3 | F03 | CTAGTTCCTATTC (SEQ ID NO: 511) | TCGTCGGCAGCGTCAGATGTGTATAAGAGACAGNNNNNNNNCTAGTTCCTATTCNNNNNN (SEQ ID NO: 512) |
| P3_64 | BacDrop _RT10X _384 _plate3 | F04 | CCCCAACCGGTGC (SEQ ID NO: 513) | TCGTCGGCAGCGTCAGATGTGTATAAGAGACAGNNNNNNNNCCCCAACCGGTGCNNNNNN (SEQ ID NO: 514) |
| P3_65 | BacDrop_RT10X _384 _plate3 | F05 | CTCTTAAAGATTG (SEQ ID NO: 515) | TCGTCGGCAGCGTCAGATGTGTATAAGAGACAGNNNNNNNNCTCTTAAAGATTGNNNNNN (SEQ ID NO: 516) |
| P3_66 | BacDrop_RT10X _384 _plate3 | F06 | CGGTCTCCTTGCG (SEQ ID NO: 517) | TCGTCGGCAGCGTCAGATGTGTATAAGAGACAGNNNNNNNNCGGTCTCCTTGCGNNNNNN (SEQ ID NO: 518) |
| P3_67 | BacDrop_RT10X _384 _plate3 | F07 | CAATGAGCAGAGG (SEQ ID NO: 519) | TCGTCGGCAGCGTCAGATGTGTATAAGAGACAGNNNNNNNNCAATGAGCAGAGGNNNNNN (SEQ ID NO: 520) |
| P3_68 | BacDrop_RT10X _384 _plate3 | F08 | CCATATTCCCTTG (SEQ ID NO: 521) | TCGTCGGCAGCGTCAGATGTGTATAAGAGACAGNNNNNNNNCCATATTCCCTTGNNNNNN (SEQ ID NO: 522) |
| P3_69 | BacDrop_RT10X _384 _plate3 | F09 | CTGTTACCCGTAG (SEQ ID NO: 523) | TCGTCGGCAGCGTCAGATGTGTATAAGAGACAGNNNNNNNNCTGTTACCCGTAGNNNNNN (SEQ ID NO: 524) |
| P3_70 | BacDrop _RT10X _384 _plate3 | F10 | CGATAATTTTCTG (SEQ ID NO: 525) | TCGTCGGCAGCGTCAGATGTGTATAAGAGACAGNNNNNNNNCGATAATTTTCTGNNNNNN (SEQ ID NO: 526) |
| P3_71 | BacDrop _RT10X _384 _plate3 | F11 | CAACCTTGAGGGG (SEQ ID NO: 527) | TCGTCGGCAGCGTCAGATGTGTATAAGAGACAGNNNNNNNNCAACCTTGAGGGGNNNNNN (SEQ ID NO: 528) |
| P3_72 | BacDrop _RT10X _384 _plate3 | F12 | CCGATAAAACGCG (SEQ ID NO: 529) | TCGTCGGCAGCGTCAGATGTGTATAAGAGACAGNNNNNNNNCCGATAAAACGCGNNNNNN (SEQ ID NO: 530) |
| P3_73 | BacDrop _RT10X _384 _plate3 | G01 | GCCTAGAGAGTTG (SEQ ID NO: 531) | TCGTCGGCAGCGTCAGATGTGTATAAGAGACAGNNNNNNNNGCCTAGAGAGTTGNNNNNN (SEQ ID NO: 532) |
| P3_74 | BacDrop _RT10X _384 _plate3 | G02 | GGTCACCATGGAG (SEQ ID NO: 533) | TCGTCGGCAGCGTCAGATGTGTATAAGAGACAGNNNNNNNNGGTCACCATGGAGNNNNNN (SEQ ID NO: 534) |
| P3_75 | BacDrop_RT10X _384 _plate3 | G03 | GCTGTGGCCCCGG (SEQ ID NO: 535) | TCGTCGGCAGCGTCAGATGTGTATAAGAGACAGNNNNNNNNGCTGTGGCCCCGGNNNNNN (SEQ ID NO: 536) |
| P3_76 | BacDrop_RT10X _384 _plate3 | G04 | GTGGGAGGCGAAG (SEQ ID NO: 537) | TCGTCGGCAGCGTCAGATGTGTATAAGAGACAGNNNNNNNNGTGGGAGGCGAAGNNNNNN (SEQ ID NO: 538) |
| P3_77 | BacDrop_RT10X _384 _plate3 | G05 | GGCTGCCGTCACG (SEQ ID NO: 539) | TCGTCGGCAGCGTCAGATGTGTATAAGAGACAGNNNNNNNNGGCTGCCGTCACGNNNNNN (SEQ ID NO: 540) |
| P3_78 | BacDrop_RT10X _384 _plate3 | G06 | GATCTTCTATGTG (SEQ ID NO: 541) | TCGTCGG CAGCGTCAGATGTGTATAAGAGACAGNNNNNNNNGATCTTCTATGTGNNNNNN (SEQ ID NO: 542) |
| P3_79 | BacDrop_RT10X _384 _plate3 | G07 | GCTACTTGCTCTG (SEQ ID NO: 543) | TCGTCGGCAGCGTCAGATGTGTATAAGAGACAGNNNNNNNNGCTACTTGCTCTGNNNNNN (SEQ ID NO: 544) |
| P3_80 | BacDrop _RT10X _384 _plate3 | G08 | GTGGGCCTTTAGG (SEQ ID NO: 545) | TCGTCGGCAGCGTCAGATGTGTATAAGAGACAGNNNNNNNNGTGGGCCTTTAGGNNNNNN (SEQ ID NO: 546) |
| P3_81 | BacDrop _RT10X _384 _plate3 | G09 | GGATACACCGGCG (SEQ ID NO: 547) | TCGTCGGCAGCGTCAGATGTGTATAAGAGACAGNNNNNNNNGGATACACCGGCGNNNNNN (SEQ ID NO: 548) |
| P3_82 | BacDrop _RT10X _384 _plate3 | G10 | GTGCGCTCGGATG (SEQ ID NO: 549) | TCGTCGGCAGCGTCAGATGTGTATAAGAGACAGNNNNNNNNGTGCGCTCGGATGNNNNNN (SEQ ID NO: 550) |
| P3_83 | BacDrop _RT10X _384 _plate3 | G11 | GCACAGGTCAAAG (SEQ ID NO: 551) | TCGTCGGCAGCGTCAGATGTGTATAAGAGACAGNNNNNNNNGCACAGGTCAAAGNNNNNN (SEQ ID NO: 552) |
| P3_84 | BacDrop _RT10X _384 _plate3 | G12 | GTCTGAGTGCCCG (SEQ ID NO: 553) | TCGTCGG CAGCGTCAGATGTGTATAAGAGACAGNNNNNNNNGTCTGAGTGCCCGNNNNNN (SEQ ID NO: 554) |
| P3_85 | BacDrop_RT10X _384 _plate3 | H01 | GGTTGTGTAGCTG (SEQ ID NO: 555) | TCGTCGGCAGCGTCAGATGTGTATAAGAGACAGNNNNNNNNGGTTGTGTAGCTGNNNNNN (SEQ ID NO: 556) |
| P3_86 | BacDrop_RT10X _384 _plate3 | H02 | GATGCGGGGCGGG (SEQ ID NO: 557) | TCGTCGGCAGCGTCAGATGTGTATAAGAGACAGNNNNNNNNGATGCGGGGCGGGNNNNNN (SEQ ID NO: 558) |
| P3_87 | BacDrop_RT10X _384 _plate3 | H03 | GCCGGTGTAAACG (SEQ ID NO: 559) | TCGTCGGCAGCGTCAGATGTGTATAAGAGACAGNNNNNNNNGCCGGTGTAAACGNNNNNN (SEQ ID NO: 560) |
| P3_88 | BacDrop_RT10X _384 _plate3 | H04 | GTGGCAAGTTCGG (SEQ ID NO: 561) | TCGTCGGCAGCGTCAGATGTGTATAAGAGACAGNNNNNNNNGTGGCAAGTTCGGNNNNNN (SEQ ID NO: 562) |
| P3_89 | BacDrop_RT10X_384_plate3 | H05 | GGTAGAATGAGTG (SEQ ID NO: 563) | TCGTCGGCAGCGTCAGATGTGTATAAGAGACAGNNNNNNNNGGTAGAATGAGTGNNNNNN (SEQ ID NO: 564) |
| P3_90 | BacDrop _RT10X _384 _plate3 | H06 | GCCCTATGGCCCG (SEQ ID NO: 565) | TCGTCGGCAGCGTCAGATGTGTATAAGAGACAGNNNNNNNNGCCCTATGGCCCGNNNNNN (SEQ ID NO: 566) |
| P3_91 | BacDrop _RT10X _384 _plate3 | H07 | GTCTATCCTCATG (SEQ ID NO: 567) | TCGTCGGCAGCGTCAGATGTGTATAAGAGACAGNNNNNNNNGTCTATCCTCATGNNNNNN (SEQ ID NO: 568) |
| P3_92 | BacDrop_RT10X_384_plate3 | H08 | GGAGTGTACTTGG (SEQ ID NO: 569) | TCGTCGGCAGCGTCAGATGTGTATAAGAGACAGNNNNNNNNGGAGTGTACTTGGNNNNNN (SEQ ID NO: 570) |
| P3_93 | BacDrop_RT10X _384 _plate3 | H09 | GATCGCGAGTACG (SEQ ID NO: 571) | TCGTCGGCAGCGTCAGATGTGTATAAGAGACAGNNNNNNNNGATCGCGAGTACGNNNNNN (SEQ ID NO: 572) |
| P3_94 | BacDrop_RT10X _384 _plate3 | H10 | GCGGTCCCAAGTG (SEQ ID NO: 573) | TCGTCGGCAGCGTCAGATGTGTATAAGAGACAGNNNNNNNNGCGGTCCCAAGTGNNNNNN (SEQ ID NO: 574) |
| P3_95 | BacDrop_RT10X _384 _plate3 | H11 | GTTCTGGGAGAGG (SEQ ID NO: 575) | TCGTCGGCAGCGTCAGATGTGTATAAGAGACAGNNNNNNNNGTTCTGGGAGAGGNNNNNN (SEQ ID NO: 576) |
| P3_96 | BacDrop_RT10X _384 _plate3 | H12 | GAACTTCGAAAAG (SEQ ID NO: 577) | TCGTCGGCAGCGTCAGATGTGTATAAGAGACAGNNNNNNNNGAACTTCGAAAAGNNNNNN (SEQ ID NO: 578) |
| P4_01 | BacDrop_RT10X_384_plate4 | A01 | AGCGTCCACACAA (SEQ ID NO: 579) | TCGTCGGCAGCGTCAGATGTGTATAAGAGACAGNNNNNNNNAGCGTCCACACAANNNNNN (SEQ ID NO: 580) |
| P4_02 | BacDrop _RT10X _384 _plate4 | A02 | ACGAGGTGCCGTA (SEQ ID NO: 581) | TCGTCGGCAGCGTCAGATGTGTATAAGAGACAGNNNNNNNNACGAGGTGCCGTANNNNNN (SEQ ID NO: 582) |
| P4_03 | BacDrop _RT10X _384 _plate4 | A03 | ATTTATTAGACGA (SEQ ID NO: 583) | TCGTCGGCAGCGTCAGATGTGTATAAGAGACAGNNNNNNNNATTTATTAGACGANNNNNN (SEQ ID NO: 584) |
| P4_04 | BacDrop _RT10X _384 _plate4 | A04 | AGGGCTCCGACAA (SEQ ID NO: 585) | TCGTCGGCAGCGTCAGATGTGTATAAGAGACAGNNNNNNNNAGGGCTCCGACAANNNNNN (SEQ ID NO: 586) |
| P4_05 | BacDrop _RT10X _384 _plate4 | A05 | AAAGTTTATCAAA (SEQ ID NO: 587) | TCGTCGGCAGCGTCAGATGTGTATAAGAGACAGNNNNNNNNAAAGTTTATCAAANNNNNN (SEQ ID NO: 588) |
| P4_06 | BacDrop_RT10X _384 _plate4 | A06 | ACACGTGTGCGGA (SEQ ID NO: 589) | TCGTCGGCAGCGTCAGATGTGTATAAGAGACAGNNNNNNNNACACGTGTGCGGANNNNNN (SEQ ID NO: 590) |
| P4_07 | BacDrop_RT10X _384 _plate4 | A07 | AACGTCAAGGATA (SEQ ID NO: 591) | TCGTCGGCAGCGTCAGATGTGTATAAGAGACAGNNNNNNNNAACGTCAAGGATANNNNNN (SEQ ID NO: 592) |
| P4_08 | BacDrop_RT10X _384 _plate4 | A08 | AGTGACAGCTACA (SEQ ID NO: 593) | TCGTCGGCAGCGTCAGATGTGTATAAGAGACAGNNNNNNNNAGTGACAGCTACANNNNNN (SEQ ID NO: 594) |
| P4_09 | BacDrop_RT10X _384 _plate4 | A09 | AAGCTTTACATGA (SEQ ID NO: 595) | TCGTCGGCAGCGTCAGATGTGTATAAGAGACAGNNNNNNNNAAGCTTTACATGANNNNNN (SEQ ID NO: 596) |
| P4_10 | BacDrop_RT10X _384 _plate4 | A10 | ATATGAACAACTA (SEQ ID NO: 597) | TCGTCGGCAGCGTCAGATGTGTATAAGAGACAGNNNNNNNNATATGAACAACTANNNNNN (SEQ ID NO: 598) |
| P4_11 | BacDrop _RT10X _384 _plate4 | A11 | AGTAGATCGCTAA (SEQ ID NO: 599) | TCGTCGGCAGCGTCAGATGTGTATAAGAGACAGNNNNNNNNAGTAGATCGCTAANNNNNN (SEQ ID NO: 600) |
| P4_12 | BacDrop _RT10X _384 _plate4 | A12 | AAAGCGTCTACCA (SEQ ID NO: 601) | TCGTCGGCAGCGTCAGATGTGTATAAGAGACAGNNNNNNNNAAAGCGTCTACCANNNNNN (SEQ ID NO: 602) |
| P4_13 | BacDrop _RT10X _384 _plate4 | B01 | ACATTTTTGAAGA (SEQ ID NO: 603) | TCGTCGGCAGCGTCAGATGTGTATAAGAGACAGNNNNNNNNACATTTTTGAAGANNNNNN (SEQ ID NO: 604) |
| P4_14 | BacDrop _RT10X _384 _plate4 | B02 | ATGTAGCTTACCA (SEQ ID NO: 605) | TCGTCGGCAGCGTCAGATGTGTATAAGAGACAGNNNNNNNNATGTAGCTTACCANNNNNN (SEQ ID NO: 606) |
| P4_15 | BacDrop _RT10X _384 _plate4 | B03 | AGTGAGCGGCAAA (SEQ ID NO: 607) | TCGTCGGCAGCGTCAGATGTGTATAAGAGACAGNNNNNNNNAGTGAGCGGCAAANNNNNN (SEQ ID NO: 608) |
| P4_16 | BacDrop_RT10X _384 _plate4 | B04 | AATGAGCCTGATA (SEQ ID NO: 609) | TCGTCGGCAGCGTCAGATGTGTATAAGAGACAGNNNNNNNNAATGAGCCTGATANNNNNN (SEQ ID NO: 610) |
| P4_17 | BacDrop_RT10X _384 _plate4 | B05 | ATCGAAACGAGGA (SEQ ID NO: 611) | TCGTCGGCAGCGTCAGATGTGTATAAGAGACAGNNNNNNNNATCGAAACGAGGANNNNNN (SEQ ID NO: 612) |
| P4_18 | BacDrop_RT10X _384 _plate4 | B06 | AGACAGTAGTCCA (SEQ ID NO: 613) | TCGTCGGCAGCGTCAGATGTGTATAAGAGACAGNNNNNNNNAGACAGTAGTCCANNNNNN (SEQ ID NO: 614) |
| P4_19 | BacDrop_RT10X _384 _plate4 | B07 | ATTTAGCTCGGTA (SEQ ID NO: 615) | TCGTCGGCAGCGTCAGATGTGTATAAGAGACAGNNNNNNNNATTTAGCTCGGTANNNNNN (SEQ ID NO: 616) |
| P4_20 | BacDrop_RT10X _384 _plate4 | B08 | AGCTTGAGGACCA (SEQ ID NO: 617) | TCGTCGGCAGCGTCAGATGTGTATAAGAGACAGNNNNNNNNAGCTTGAGGACCANNNNNN (SEQ ID NO: 618) |
| P4_21 | BacDrop _RT10X _384 _plate4 | B09 | AACAGACGCCTAA (SEQ ID NO: 619) | TCGTCGGCAGCGTCAGATGTGTATAAGAGACAGNNNNNNNNAACAGACGCCTAANNNNNN (SEQ ID NO: 620) |
| P4_22 | BacDrop _RT10X _384 _plate4 | B10 | ATTGGACAGGGCA (SEQ ID NO: 621) | TCGTCGGCAGCGTCAGATGTGTATAAGAGACAGNNNNNNNNATTGGACAGGGCANNNNNN (SEQ ID NO: 622) |
| P4_23 | BacDrop _RT10X _384 _plate4 | B11 | AAAATTCAGTATA (SEQ ID NO: 623) | TCGTCGGCAGCGTCAGATGTGTATAAGAGACAGNNNNNNNNAAAATTCAGTATANNNNNN (SEQ ID NO: 624) |
| P4_24 | BacDrop _RT10X _384 _plate4 | B12 | ACTGTCGAAATAA (SEQ ID NO: 625) | TCGTCGGCAGCGTCAGATGTGTATAAGAGACAGNNNNNNNNACTGTCGAAATAANNNNNN (SEQ ID NO: 626) |
| P4_25 | BacDrop _RT10X _384 _plate4 | C01 | TTGGGTATCAGGA (SEQ ID NO: 627) | TCGTCGGCAGCGTCAGATGTGTATAAGAGACAGNNNNNNNNTTGGGTATCAGGANNNNNN (SEQ ID NO: 628) |
| P4_26 | BacDrop_RT10X _384 _plate4 | C02 | TGCTATCAGTCTA (SEQ ID NO: 629) | TCGTCGGCAGCGTCAGATGTGTATAAGAGACAGNNNNNNNNTGCTATCAGTCTANNNNNN (SEQ ID NO: 630) |
| P4_27 | BacDrop_RT10X _384 _plate4 | C03 | TAAGGCGGTAAGA (SEQ ID NO: 631) | TCGTCGGCAGCGTCAGATGTGTATAAGAGACAGNNNNNNNNTAAGGCGGTAAGANNNNNN (SEQ ID NO: 632) |
| P4_28 | BacDrop_RT10X _384 _plate4 | C04 | TCGATTGGGTTTA (SEQ ID NO: 633) | TCGTCGGCAGCGTCAGATGTGTATAAGAGACAGNNNNNNNNTCGATTGGGTTTANNNNNN (SEQ ID NO: 634) |
| P4_29 | BacDrop_RT10X _384 _plate4 | C05 | TTGCCCAGCATAA (SEQ ID NO: 635) | TCGTCGGCAGCGTCAGATGTGTATAAGAGACAGNNNNNNNNTTGCCCAGCATAANNNNNN (SEQ ID NO: 636) |
| P4_30 | BacDrop_RT10X_384_plate4 | C06 | TGCACTGGGCGCA (SEQ ID NO: 637) | TCGTCGGCAGCGTCAGATGTGTATAAGAGACAGNNNNNNNNTGCACTGGGCGCANNNNNN (SEQ ID NO: 638) |
| P4_31 | BacDrop_RT10X_384_plate4 | C07 | TATGGGAATAAAA (SEQ ID NO: 639) | TCGTCGGCAGCGTCAGATGTGTATAAGAGACAGNNNNNNNNTATGGGAATAAAANNNNNN (SEQ ID NO: 640) |
| P4_32 | BacDrop_RT10X_384_plate4 | C08 | TCTACCCGGTTGA (SEQ ID NO: 641) | TCGTCGGCAGCGTCAGATGTGTATAAGAGACAGNNNNNNNNTCTACCCGGTTGANNNNNN (SEQ ID NO: 642) |
| P4_33 | BacDrop_RT10X_384_plate4 | C09 | TCATCCAGATCAC (SEQ ID NO: 643) | TCGTCGGCAGCGTCAGATGTGTATAAGAGACAGNNNNNNNNTCATCCAGATCACNNNNNN (SEQ ID NO: 644) |
| P4_34 | BacDrop_RT10X_384_plate4 | C10 | TGAAGCAATTAGC (SEQ ID NO: 645) | TCGTCGGCAGCGTCAGATGTGTATAAGAGACAGNNNNNNNNTGAAGCAATTAGCNNNNNN (SEQ ID NO: 646) |
| P4_35 | BacDrop_RT10X_384_plate4 | C11 | TATGGCGTCCTTC (SEQ ID NO: 647) | TCGTCGGCAGCGTCAGATGTGTATAAGAGACAGNNNNNNNNTATGGCGTCCTTCNNNNNN (SEQ ID NO: 648) |
| P4_36 | BacDrop_RT10X_384_plate4 | C12 | TCAATATATTGAC (SEQ ID NO: 649) | TCGTCGGCAGCGTCAGATGTGTATAAGAGACAGNNNNNNNNTCAATATATTGACNNNNNN (SEQ ID NO: 650) |
| P4_37 | BacDrop_RT10X_384_plate4 | D01 | TTTGGACGACCGC (SEQ ID NO: 651) | TCGTCGGCAGCGTCAGATGTGTATAAGAGACAGNNNNNNNNTTTGGACGACCGCNNNNNN (SEQ ID NO: 652) |
| P4_38 | BacDrop_RT10X_384_plate4 | D02 | TGATTGACCAAAC (SEQ ID NO: 653) | TCGTCGGCAGCGTCAGATGTGTATAAGAGACAGNNNNNNNNTGATTGACCAAACNNNNNN (SEQ ID NO: 654) |
| P4_39 | BacDrop_RT10X_384_plate4 | D03 | TCGAACATACCTC (SEQ ID NO: 655) | TCGTCGGCAGCGTCAGATGTGTATAAGAGACAGNNNNNNNNTCGAACATACCTCNNNNNN (SEQ ID NO: 656) |
| P4_40 | BacDrop_RT10X_384_plate4 | D04 | TTTCATCCAATAC (SEQ ID NO: 657) | TCGTCGGCAGCGTCAGATGTGTATAAGAGACAGNNNNNNNNTTTCATCCAATACNNNNNN (SEQ ID NO: 658) |
| P4_41 | BacDrop_RT10X_384_plate4 | D05 | TGCGGGAAAGGCC (SEQ ID NO: 659) | TCGTCGGCAGCGTCAGATGTGTATAAGAGACAGNNNNNNNNTGCGGGAAAGGCCNNNNNN (SEQ ID NO: 660) |
| P4_42 | BacDrop_RT10X_384_plate4 | D06 | TCAGGGATCACTC (SEQ ID NO: 661) | TCGTCGGCAGCGTCAGATGTGTATAAGAGACAGNNNNNNNNTCAGGGATCACTCNNNNNN (SEQ ID NO: 662) |
| P4_43 | BacDrop_RT10X_384_plate4 | D07 | TGTGTTACTTCCC (SEQ ID NO: 663) | TCGTCGGCAGCGTCAGATGTGTATAAGAGACAGNNNNNNNNTGTGTTACTTCCCNNNNNN (SEQ ID NO: 664) |
| P4_44 | BacDrop_RT10X_384_plate4 | D08 | TACGGGTGTTGAC (SEQ ID NO: 665) | TCGTCGGCAGCGTCAGATGTGTATAAGAGACAGNNNNNNNNTACGGGTGTTGACNNNNNN (SEQ ID NO: 666) |
| P4_45 | BacDrop_RT10X_384_plate4 | D09 | TCCTGATGGTAGC (SEQ ID NO: 667) | TCGTCGGCAGCGTCAGATGTGTATAAGAGACAGNNNNNNNNTCCTGATGGTAGCNNNNNN (SEQ ID NO: 668) |
| P4_46 | BacDrop_RT10X_384_plate4 | D10 | TTTCGTGCTTTCC (SEQ ID NO: 669) | TCGTCGGCAGCGTCAGATGTGTATAAGAGACAGNNNNNNNNTTTCGTGCTTTCCNNNNNN (SEQ ID NO: 670) |
| P4_47 | BacDrop_RT10X_384_plate4 | D11 | TGACGCGACGATC (SEQ ID NO: 671) | TCGTCGGCAGCGTCAGATGTGTATAAGAGACAGNNNNNNNNTGACGCGACGATCNNNNNN (SEQ ID NO: 672) |
| P4_48 | BacDrop_RT10X_384_plate4 | D12 | TATTGTTAACGAC (SEQ ID NO: 673) | TCGTCGGCAGCGTCAGATGTGTATAAGAGACAGNNNNNNNNTATTGTTAACGACNNNNNN (SEQ ID NO: 674) |
| P4_49 | BacDrop_RT10X_384_plate4 | E01 | CCAAGCGCGTCCC (SEQ ID NO: 675) | TCGTCGGCAGCGTCAGATGTGTATAAGAGACAGNNNNNNNNCCAAGCGCGTCCCNNNNNN (SEQ ID NO: 676) |
| P4_50 | BacDrop_RT10X_384_plate4 | E02 | CTTATCGTCAAGC (SEQ ID NO: 677) | TCGTCGGCAGCGTCAGATGTGTATAAGAGACAGNNNNNNNNCTTATCGTCAAGCNNNNNN (SEQ ID NO: 678) |
| P4_51 | BacDrop_RT10X_384_plate4 | E03 | CCTAGAAGTCGAC (SEQ ID NO: 679) | TCGTCGGCAGCGTCAGATGTGTATAAGAGACAGNNNNNNNNCCTAGAAGTCGACNNNNNN (SEQ ID NO: 680) |
| P4_52 | BacDrop_RT10X_384_plate4 | E04 | CTCGGCATGATCC (SEQ ID NO: 681) | TCGTCGGCAGCGTCAGATGTGTATAAGAGACAGNNNNNNNNCTCGGCATGATCCNNNNNN (SEQ ID NO: 682) |
| P4_53 | BacDrop_RT10X_384_plate4 | E05 | CGACGGCGCACAC (SEQ ID NO: 683) | TCGTCGGCAGCGTCAGATGTGTATAAGAGACAGNNNNNNNNCGACGGCGCACACNNNNNN (SEQ ID NO: 684) |
| P4_54 | BacDrop_RT10X_384_plate4 | E06 | CAGACCGTGGACC (SEQ ID NO: 685) | TCGTCGGCAGCGTCAGATGTGTATAAGAGACAGNNNNNNNNCAGACCGTGGACCNNNNNN (SEQ ID NO: 686) |
| P4_55 | BacDrop_RT10X_384_plate4 | E07 | CCAGGTGACGGCC (SEQ ID NO: 687) | TCGTCGGCAGCGTCAGATGTGTATAAGAGACAGNNNNNNNNCCAGGTGACGGCCNNNNNN (SEQ ID NO: 688) |
| P4_56 | BacDrop_RT10X_384_plate4 | E08 | CTCGTGTCGGAGC (SEQ ID NO: 689) | TCGTCGGCAGCGTCAGATGTGTATAAGAGACAGNNNNNNNNCTCGTGTCGGAGCNNNNNN (SEQ ID NO: 690) |
| P4_57 | BacDrop_RT10X_384_plate4 | E09 | CTTTCGTGTAGAC (SEQ ID NO: 691) | TCGTCGGCAGCGTCAGATGTGTATAAGAGACAC CT T TCGTGTTAGACNNNNNN (SEQ ID NO: 692) |
| P4_58 | BacDrop_RT10X_384_plate4 | E10 | CGCCAGTGCTTTC (SEQ ID NO: 693) | TCGTCGGCAGCGTCAGATGTGTATAAGAGACAGNNNNNNNNCGCCAGTGCTTTCNNNNNN (SEQ ID NO: 694) |
| P4_59 | BacDrop_RT10X_384_plate4 | E11 | CATGTTGCGGGAC (SEQ ID NO: 695) | TCGTCGGCAGCGTCAGATGTGTATAAGAGACAGNNNNNNNNCATGTTGCGGGACNNNNNN (SEQ ID NO: 696) |
| P4_60 | BacDrop_RT10X_384_plate4 | E12 | CCGCTTGTATAGC (SEQ ID NO: 697) | TCGTCGGCAGCGTCAGATGTGTATAAGAGACAGNNNNNNNNCCGCTTGTATAGCNNNNNN (SEQ ID NO: 698) |
| P4_61 | BacDrop_RT10X_384_plate4 | F01 | CTCGTCACTGGCC (SEQ ID NO: 699) | TCGTCGGCAGCGTCAGATGTGTATAAGAGACAGNNNNNNNNCTCGTCACTGGCCNNNNNN (SEQ ID NO: 700) |
| P4_62 | BacDrop_RT10X_384_plate4 | F02 | CGCTTTTACTGAC (SEQ ID NO: 701) | TCGTCGGCAGCGTCAGATGTGTATAAGAGACAGNNNNNNNNCGCTTTTACTGACNNNNNN (SEQ ID NO: 702) |
| P4_63 | BacDrop_RT10X_384_plate4 | F03 | CAGCAGGGACGCC (SEQ ID NO: 703) | TCGTCGGCAGCGTCAGATGTGTATAAGAGACAGNNNNNNNNCAGCAGGGACGCCNNNNNN (SEQ ID NO: 704) |
| P4_64 | BacDrop_RT10X_384_plate4 | F04 | CCCGCTCGACTAC (SEQ ID NO: 705) | TCGTCGGCAGCGTCAGATGTGTATAAGAGACAGNNNNNNNNCCCGCTCGACTACNNNNNN (SEQ ID NO: 706) |
| P4_65 | BacDrop_RT10X_384_plate4 | F05 | CTTCTGATTTTGG (SEQ ID NO: 707) | TCGTCGGCAGCGTCAGATGTGTATAAGAGACAGNNNNNNNNCTTCTGATTTTGGNNNNNN (SEQ ID NO: 708) |
| P4_66 | BacDrop_RT10X_384_plate4 | F06 | CGATGTGCGTGTG (SEQ ID NO: 709) | TCGTCGGCAGCGTCAGATGTGTATAAGAGACAGNNNNNNNNCGATGTGCGTGTGNNNNNN (SEQ ID NO: 710) |
| P4_67 | BacDrop_RT10X_384_plate4 | F07 | CACCCCGCTCCTG (SEQ ID NO: 711) | TCGTCGGCAGCGTCAGATGTGTATAAGAGACAGNNNNNNNNCACCCCGCTCCTGNNNNNN (SEQ ID NO: 712) |
| P4_68 | BacDrop_RT10X_384_plate4 | F08 | CCAGATTGTAACG (SEQ ID NO: 713) | TCGTCGGCAGCGTCAGATGTGTATAAGAGACAGNNNNNNNNCCAGATTGTAACGNNNNNN (SEQ ID NO: 714) |
| P4_69 | BacDrop_RT10X_384_plate4 | F09 | CTGCTCCACCAAG (SEQ ID NO: 715) | TCGTCGGCAGCGTCAGATGTGTATAAGAGACAGNNNNNNNNCTGCTCCACCAAGNNNNNN (SEQ ID NO: 716) |
| P4_70 | BacDrop_RT10X_384_plate4 | F10 | CGCGATATTCGGG (SEQ ID NO: 717) | TCGTCGGCAGCGTCAGATGTGTATAAGAGACAGNNNNNNNNCGCGATATTCGGGNNNNNN (SEQ ID NO: 718) |
| P4_71 | BacDrop_RT10X_384_plate4 | F11 | CACAGGGGCAACG (SEQ ID NO: 719) | TCGTCGGCAGCGTCAGATGTGTATAAGAGACAGNNNNNNNNCACAGGGGCAACGNNNNNN (SEQ ID NO: 720) |
| P4_72 | BacDrop_RT10X_384_plate4 | F12 | CCTCTGATCCGTG (SEQ ID NO: 721) | TCGTCGGCAGCGTCAGATGTGTATAAGAGACAGNNNNNNNNCCTCTGATCCGTGNNNNNN (SEQ ID NO: 722) |
| P4_73 | BacDrop_RT10X_384_plate4 | G01 | GTCATTGACTTGG (SEQ ID NO: 723) | TCGTCGGCAGCGTCAGATGTGTATAAGAGACAGNNNNNNNNGTCATTGACTTGGNNNNNN (SEQ ID NO: 724) |
| P4_74 | BacDrop_RT10X_384_plate4 | G02 | GGGAAATCGTACG (SEQ ID NO: 725) | TCGTCGGCAGCGTCAGATGTGTATAAGAGACAGNNNNNNNNGGGAAATCGTACGNNNNNN (SEQ ID NO: 726) |
| P4_75 | BacDrop_RT10X_384_plate4 | G03 | GAACTTACTGCGG (SEQ ID NO: 727) | TCGTCGGCAGCGTCAGATGTGTATAAGAGACAGNNNNNNNNGAACTTACTGCGGNNNNNN (SEQ ID NO: 728) |
| P4_76 | BacDrop_RT10X_384_plate4 | G04 | GCGACGGATTAAG (SEQ ID NO: 729) | TCGTCGGCAGCGTCAGATGTGTATAAGAGACAGNNNNNNNNGCGACGGATTAAGNNNNNN (SEQ ID NO: 730) |
| P4_77 | BacDrop_RT10X_384_plate4 | G05 | GTTGCGATGTCCG (SEQ ID NO: 731) | TCGTCGGCAGCGTCAGATGTGTATAAGAGACAGNNNNNNNNGTTGCGATGTCCGNNNNNN (SEQ ID NO: 732) |
| P4_78 | BacDrop_RT10X_384_plate4 | G06 | GGGATCGCACAGG (SEQ ID NO: 733) | TCGTCGGCAGCGTCAGATGTGTATAAGAGACAGNNNNNNNNGGGATCGCACAGGNNNNNN (SEQ ID NO: 734) |
| P4_79 | BacDrop_RT10X_384_plate4 | G07 | GACCAAATTGTAG (SEQ ID NO: 735) | TCGTCGGCAGCGTCAGATGTGTATAAGAGACAGNNNNNNNNGACCAAATTGTAGNNNNNN (SEQ ID NO: 736) |
| P4_80 | BacDrop_RT10X_384_plate4 | G08 | GCTGGTCAGTTTG (SEQ ID NO: 737) | TCGTCGGCAGCGTCAGATGTGTATAAGAGACAGNNNNNNNNGCTGGTCAGTTTGNNNNNN (SEQ ID NO: 738) |
| P4_81 | BacDrop_RT10X_384_plate4 | G09 | GGCCAAGGCGCCG (SEQ ID NO: 739) | TCGTCGGCAGCGTCAGATGTGTATAAGAGACAGNNNNNNNNGGCCAAGGCGCCGNNNNNN (SEQ ID NO: 740) |
| P4_82 | BacDrop_RT10X_384_plate4 | G10 | GATGTAAGTCTTG (SEQ ID NO: 741) | TCGTCGGCAGCGTCAGATGTGTATAAGAGACAGNNNNNNNNGATGTAAGTCTTGNNNNNN (SEQ ID NO: 742) |
| P4_83 | BacDrop_RT10X_384_plate4 | G11 | GGGTGCTCCACAG (SEQ ID NO: 743) | TCGTCGGCAGCGTCAGATGTGTATAAGAGACAGNNNNNNNNGGGTGCTCCACAGNNNNNN (SEQ ID NO: 744) |
| P4_84 | BacDrop_RT10X_384_plate4 | G12 | GCTGGTAGAGGTG (SEQ ID NO: 745) | TCGTCGGCAGCGTCAGATGTGTATAAGAGACAGNNNNNNNNGCTGGTAGAGGTGNNNNNN (SEQ ID NO: 746) |
| P4_85 | BacDrop_RT10X_384_plate4 | H01 | GGCAGATTCCAGG (SEQ ID NO: 747) | TCGTCGGCAGCGTCAGATGTGTATAAGAGACAGNNNNNNNNGGCAGATTCCAGGNNNNNN (SEQ ID NO: 748) |
| P4_86 | BacDrop_RT10X_384_plate4 | H02 | GATGGTTGCAATG (SEQ ID NO: 749) | TCGTCGGCAGCGTCAGATGTGTATAAGAGACAGNNNNNNNNGATGGTTGCAATGNNNNNN (SEQ ID NO: 750) |
| P4_87 | BacDrop_RT10X_384_plate4 | H03 | GGCGGGTAGATAG (SEQ ID NO: 751) | TCGTCGGCAGCGTCAGATGTGTATAAGAGACAGNNNNNNNNGGCGGGTAGATAGNNNNNN (SEQ ID NO: 752) |
| P4_88 | BacDrop_RT10X_384_plate4 | H04 | GCCACTCTTGGGG (SEQ ID NO: 753) | TCGTCGGCAGCGTCAGATGTGTATAAGAGACAGNNNNNNNNGCCACTCTTGGGGNNNNNN (SEQ ID NO: 754) |
| P4_89 | BacDrop_RT10X_384_plate4 | H05 | GGTGAGGTATGTG (SEQ ID NO: 755) | TCGTCGGCAGCGTCAGATGTGTATAAGAGACAGNNNNNNNNGGTGAGGTATGTGNNNNNN (SEQ ID NO: 756) |
| P4_90 | BacDrop_RT10X_384_plate4 | H06 | GAACGTTTATACG (SEQ ID NO: 757) | TCGTCGGCAGCGTCAGATGTGTATAAGAGACAGNNNNNNNNGAACGTTTATACGNNNNNN (SEQ ID NO: 758) |
| P4_91 | BacDrop_RT10X_384_plate4 | H07 | GGTGCCGCCAGCG (SEQ ID NO: 759) | TCGTCGGCAGCGTCAGATGTGTATAAGAGACAGNNNNNNNNGGTGCCGCCAGCGNNNNNN (SEQ ID NO: 760) |
| P4_92 | BacDrop_RT10X_384_plate4 | H08 | GCGCGTTACTCAG (SEQ ID NO: 761) | TCGTCGGCAGCGTCAGATGTGTATAAGAGACAGNNNNNNNNGCGCGTTACTCAGNNNNNN (SEQ ID NO: 762) |
| P4_93 | BacDrop_RT10X_384_plate4 | H09 | GTGATTGATGGAG (SEQ ID NO: 763) | TCGTCGGCAGCGTCAGATGTGTATAAGAGACAGNNNNNNNNGTGATTGATGGAGNNNNNN (SEQ ID NO: 764) |
| P4_94 | BacDrop_RT10X_384_plate4 | H10 | GGTTACGGGCCGG (SEQ ID NO: 765) | TCGTCGGCAGCGTCAGATGTGTATAAGAGACAGNNNNNNNNGGTTACGGGCCGGNNNNNN (SEQ ID NO: 766) |
| P4_95 | BacDrop_RT10X_384_plate4 | H11 | GACCAGCATACAG (SEQ ID NO: 767) | TCGTCGGCAGCGTCAGATGTGTATAAGAGACAGNNNNNNNNGACCAGCATACAGNNNNNN (SEQ ID NO: 768) |
| P4_96 | BacDrop_RT10X_384_plate4 | H12 | GCGAGTTTAATGG (SEQ ID NO: 769) | TCGTCGGCAGCGTCAGATGTGTATAAGAGACAGNNNNNNNNGCGAGTTTAATGGNNNNNN (SEQ ID NO: 770) |

TABLE 2

Bacterial Strains and plasmids used in this study

| Strain Name | Species | Description | MICs of Meropenem (ug/mL) | MICs of Ciprofloxacin (ug/mL) | MICs of Gentamicin (ug/mL) | Reference | Accession no. |
|---|---|---|---|---|---|---|---|
| MGH66 | K. pneumoniae | A K. pneumoniae clinical isolate, susceptible to carbapenems, ciprofloxacin, and gentamicin | 0.03 | 0.03 | 1 | Ma et al., eLife, 2021 | GCA_00069455 5.1 |
| BIDMC35 | K. pneumoniae | A carbapenemresistant K. pneumoniae clinical isolate | >32 | >32 | 0.5 | Ma et al., AAC, 2018 | GCA_00056722 5.2 |
| UCI38 | K. pneumoniae | A K. pneumoniae clinical isolate, susceptible to carbapenems, resistant to ciprofloxacin and gentamicin | 0.03 | 32 | 2 | Ma et al., eLife, 2021 | GCA_00056680 5.1 |
| E. coli 10ß | E. coli | E. coli strain from NEB | <0.03 | <0.03 | <0.03 | NEB | GCF_00000886 5.2 |
| PAO1 | P. aeruginosa | A P. aeruginosa pathogenic isolate | Not tested | Not tested | Not tested | Stover et al., Nature 2000 | GCF_00000676 5.1 |
| EnGen0052-E3346 | E. faecium | An E. faecium human commensal isolate | Not tested | Not tested | Not tested | Lebreton et al., Cell, 2017 | GCA_00032228 5.1 |
| gfp.low | E. coli | E. coli expressing a GFP reporter driven by the promoter of spy | Not tested | Not tested | Not tested | Zaslaver, A. et al, Nature Method, 2006 | |
| gfp.mid | E. coli | E. coli expressing a GFP reporter driven by the promoter of rpoH | Not tested | Not tested | Not tested | Zaslaver, A. et al, Nature Method, 2006 | |
| gfp.high | E. coli | E. coli expressing a GFP reporter driven by the promoter of rpsM | Not tested | Not tested | Not tested | Zaslaver, A. et al, Nature Method, 2006 | |
| MGH66: PyhcN:gfp | K. pneumoniae | MGH66 expressing a GFP reporter driven by the promoter of yhcN | 0.03 | 0.03 | 1 | This study | |
| MGH66: PcpsD:gfp | K. pneumoniae | MGH66 expressing a GFP reporter driven by the promoter of cspD | 0.03 | 0.03 | 1 | This study | |
| pUA139 | plasmid | | | | | Zaslaver, A. et al, Nature Method, 2006 | |
| pUA139-PyhcN:gfp | plasmid | | | | | This study | |
| pUA139-PcspD:gfp | plasmid | | | | | This study | |
| MGH66:pBA D: cspD | K. pneumoniae | MGH66 transformed with pBAD:cspD | 0.03 | Not tested | Not tested | This study | |

TABLE 3

Prophage predicted in BIDMC35

| Candidate ID | SequenceID | Start | End | Length | Category | Score | Closest phage | Gene number |
|---|---|---|---|---|---|---|---|---|
| Candidate_34 | JCMW02000001.1 | 2417098 | 2452250 | 35153 | Active | 0.99 | Escherichia phage TL-2011b | 40 |
| Candidate_86 | JCMW02000004.1 | 10520 | 88007 | 77488 | Active | 0.88 | Salmonella phage SSU5 | 81 |

TABLE 4

Markers gene identified from significant clusters of the meropenem treated sample

| GeneID | Gene product | Pathway | Biological processes | Cluster | p_val | avg_lo g2FC | pct.1 | pct.2 | p_val_ adj |
|---|---|---|---|---|---|---|---|---|---|
| cds-WP-032430210.1 | PBP3 | Target of beta-lactams | Target of beta-lactams | 9 | 0.00E+ 00 | 5.2957 | 0.712 | 0.013 | 0.00E+ 00 |
| cds-WP-032429893.1 | PBP6 | Target of beta-lactams | Target of beta-lactams | 5_12 | 5.46E-44 | 3.7036 599 | 0.257 | 0.01 | 1.81E-40 |
| cds-WP-002921264.1 | DtpB | Transportation of beta-lactams | Dipeptide/Tripeptide Transport | 7_11 | 0.00E+ 00 | 6.2815 94 | 0.619 | 0.004 | 0.00E+ 00 |
| cds-WP-002913995.1 | SuhB | Membrane protein transport and insertion | Cell wall/membrane synthesis | 7_11 | 4.19E-129 | 4.4065 98 | 0.381 | 0.008 | 1.39E-125 |
| cds-WP-004151320.1 | LpxH | Lipid A biosynthesis | Cell wall/membrane synthesis | 7_11 | 0.00E+ 00 | 7.6053 74 | 0.976 | 0.002 | 0.00E+ 00 |
| cds-WP-002918241.1 | Nlpl | Cell wall synthesis, bind to PBP4 and PBP1A | Cell wall/membrane synthesis | 7_11 | 7.64E-91 | 3.4877 22 | 0.833 | 0.059 | 2.54E-87 |
| cds-WP-002883303.1 | RffG | LPS synthesis | Cell wall/membrane synthesis | 7_11 | 1.80E-110 | 4.2802 85 | 0.476 | 0.015 | 5.97E-107 |
| cds-WP-004149864.1 | DnaG | DNA replication | DNA replication | 9 | 0.00E+ 00 | 4.4907 15 | 0.862 | 0.029 | 0.00E+ 00 |
| cds-WP-004150295.1 | YidC | Membrane damage response | Stress response | 5_12 | 2.48E-103 | 4.0687 447 | 1 | 0.068 | 8.25E-100 |
| cds-WP-004217130.1 | Spy | Membrane damage response | Stress response | 5_12 | 7.00E-102 | 4.0142 941 | 0.124 | 0.007 | 2.33E-98 |
| cds-WP-002914070.1 | RseB | Membrane damage response | Stress response | 5_12 | 6.81E-222 | 5.1874 707 | 0.886 | 0.022 | 2.26E-218 |
| cds-WP-032428421.1 | YhcN | Cytoplasmic acid stress | Stress response | 5_12 | 0.00E+ 00 | 6.1654 092 | 0.486 | 0.003 | 0.00E+ 00 |
| cds-WP-032429693.1 | HscA | Cold shock | Stress response | 5_12 | 1.73E-29 | 3.2262 902 | 0.257 | 0.015 | 5.75E-26 |
| cds-WP-002903315.1 | FumC | Oxidative stress | Stress response | 9 | 0.00E+ 00 | 5.3042 57 | 0.262 | 0.001 | 0.00E+ 00 |
| cds-WP-002910443.1 | YchF | Oxidative stress | Stress response | 9 | 0.00E+ 00 | 5.2866 79 | 0.912 | 0.017 | 0.00E+ 00 |
| cds-WP-004152420.1 | GroES | Stress response | Stress response | 9 | 7.24E-265 | 4.5107 94 | 0.588 | 0.016 | 2.41E-261 |
| cds-WP-032430200.1 | RihC | DNA damage response | Stress response | 10 | 0.00E+ 00 | 6.1612 2 | 0.472 | 0.002 | 0.00E+ 00 |
| cds-WP-002896516.1 | CspD | Persister formation | DNA replication inhibition | 10 | 0.00E+ 00 | 6.3423 85 | 0.509 | 0.001 | 0.00E+ 00 |
| cds-WP-013815099.1-4 | IS903B | Insertion sequence | Mobile genetic elements | 4_6_8 | 0 | 4.4631 17 | 0.489 | 0.017 | 0 |

TABLE 5

Reagent lists and cost

| | Item | Supplier | Supplier | PN/CN | Cost per 1 sample (USD, estimated) | Cost per cell (~1e6 cells/sample; USD, estimated) |
|---|---|---|---|---|---|---|
| A1) Fixation and permeabilization, with rRNA and gDNA depletion | | | | | | |
| | NxGen RNase Inhibitor | Lucigen | Lucigen | 30281-2 | 60 | 0.00006 |
| | Lysozyme | ThermoFisher | ThermoFisher | 90082 | | |
| | NEBNext rRNA Depletion Kit | NEB | NEB | E7850X | | |
| | DNase I | Sigma | Sigma | AMPD1 | | |
| | SUPERase-In RNase inhibitor | ThermoFishe r | ThermoFisher | AM2696 | | |
| A2) In-cell reverse transcription, R1 barcoding, and poly-A tailing | | | | | | |
| | Round 1 Barcodes | IDT | IDT | | 500 | 0.0005 |
| | Maxima H Minus reverse transcriptase | Thermo Scientific | ThermoFisher | EP0753 | | |
| | DTT | ?? | ProMega | P1171 | | |
| | dNTPs | ?? | NEB | N0447L | | |
| | Terminal transferase (TdT) | NEB | NEB | M0315L | | |
| | dATP | NEB | NEB | N0446S | | |
| | SUPERase-In RNase inhibitor | ThermoFishe r | ThermoFisher | AM2696 | | |
| A3) Droplet generation and R2 barcoding | | | | | | |
| | Chromium Next GEM Chip H | 10X Genomics | 10X Genomics | 1000161 | 1500 | 0.0015 |
| | Chromium Next GEM Single Cell ATAC Library & Gel Bead Kit | 10X Genomics | 10X Genomics | 1000176 | | |
| | KAPA HiFi HotStart ReadyMix | Roche | Roche | KK2602 | | |
| | 5% FC-40 oil | RAN Biotechnolog ies | RAN Biotechnologies | 008-FluoroSurf actant-5wtF | | |
| | SMRT_dT | IDT?? | IDT | | | |
| A4) cDNA amplification and Illumina library construction | | | | | | |
| | Buffer EB | Qiagen | Qiagen | 19086 | 200 | 0.0002 |
| | Primer P5 | | IDT | | | |
| | Primer SMRT_PCR | | IDT | | | |
| | 5X SYBR Green | VWR | VWR | 12001-796 | | |
| | Illumina Nextera XT DNA library preparation kit | Illumina | Illumina | FC-131-1096 | | |
| | Primer P5 | IDT | IDT | | | |
| | Nextera index 1 primer | Nextera | Illumina | FC-131-2001 | | |
| | AMPure XP beads | Beckman Coulter | Beckman Coulter | A63881 | | |
| B) Sequencing | | | | | | |
| | NovaSeq 6000 SP | Illumina | Illumina | | 3250 | 0.00325 |
| Total Costs | | | | | 5510 | 0.00551 |

TABLE 6

Other primers used in this study

| | | |
|---|---|---|
| Primer Name | Sequence | Comments |
| SMRT_dT | AAGCAGTGGTATCAACGCAGAGTTTTTTTTTTTTTTTVN (SEQ ID NO: 771) | 2nd strand synthesis after TdT treatment with dATP |
| SMRT_PCR | AAGCAGTGGTATCAACGCAGAGT (SEQ ID NO: 772) | used for cDNA amplification |
| P5 | AATGATACGGCGACCACCGAGA (SEQ ID NO: 773) | P5 partial primer, used for cDNA amplification (paired with SMRT_PCR) and for library construction (paired with Nextera index primers N7**) |
| cspD_F | ACTGGGATCCATGCAGATGGCGGTTTAATCGCAT (SEQ ID NO: 774) | Primers used to amplify the promoter of cspD from MGH66 and ligate into pUA139. |
| cspD_R | ACTGCTCGAGATGCCAT ACT TCG ACA TCC TTC GT (SEQ ID NO: 775) | |
| YhcN_F | ACTGGGATCCATGCAAGCGTTGTGATGAGAACAT (SEQ ID NO: 776) | Primers used to amplify the promoter of cspD from MGH66 and ligate into pUA139. |
| YhcN_R | ACTGCTCGAGATGCGAT GTT CAC CTC GTC GAA TC (SEQ ID NO: 777) | |

TABLE 7

Number of PC used in the analysis

| Experiment | Sample_name | Sample_type | Downsample | Number of cells in the analysis | min.Feature | Number of PC |
|---|---|---|---|---|---|---|
| MGH66_combined | MGH66_combined | between population | No | ~60k | 15 | 9 |
| MGH66_combined | MGH66_combined | between population | 50% | ~30k | 15 | 10 |
| MGH66_combined | MGH66_combined | between population | 25% | ~15k | 15 | 5 |
| MGH66_combined | MGH66_combined | between population | 10% | ~7k | 15 | 5 |
| MGH66_combined | MGH66_combined | between population | No | ~150k | 10 | 10 |
| MGH66_combined | MGH66_combined | between population | 50% | ~70k | 10 | 10 |
| MGH66 | MGH66_untreated | within population | No | ~40k | 15 | 5 |
| MGH66 | MGH66_untreated | within population | 50% | ~20k | 15 | 5 |
| MGH66 | MGH66_untreated | within population | 25% | ~10k | 15 | 7 |
| MGH66 | MGH66_meropenem | within population | No | ~10k | 15 | 5 |
| BIDMC35 | BIDMC35 | within population | No | ~10k | 10 | 10 |

TABLE 8

Counts table of the E. coli library with 10k cells. Table organized as Geneid, Chr, Start, End, Strand, Length, E. coli_10k; Geneid, Chr, Start, End, Strand, Length, E. coli_10k; etc
cds-WP_001386572.1, NZ_CP025268.1, 190, 255, +, 66, 0; cds-WP_001264707.1, NZ_CP025268.1, 337, 2799, +, 2463, 1181867; cds-WP_000241662.1, NZ_CP025268.1, 2801, 3733, +, 933, 57154; cds-WP_000781074.1, NZ_CP025268.1, 3734, 5020, +, 1287, 102096; cds-WP_000738719.1, NZ_CP025268.1, 5234, 5530, +, 297, 32407; cds-WP_000906197.1, NZ_CP025268.1, 5683, 6459, -, 777, 436; cds-CXP41_RS00035, NZ_CP025268.1, 6529, 7960, -, 1432, 0; cds-WP_000130185.1, NZ_CP025268.1, 8239, 9192, +, 954, 137861; cds-WP_001295414.1, NZ_CP025268.1, 9307, 9894, +, 588, 171445; cds-WP_000528538.1, NZ_CP025268.1, 9929, 10495, -, 567, 246268; cds-WP_101348525.1, NZ_CP025268.1, 10644, 11357, -, 714, 58514; cds-WP_000843565.1, NZ_CP025268.1, 11383, 11787, -, 405, 0; cds-WP_000516135.1, NZ_CP025268.1, 12164, 14080, +, 1917, 2865083; cds-WP_101348526.1, NZ_CP025268.1, 14169, 15299, +, 1131, 1239622; cds-WP_001300563.1, NZ_CP025268.1, 15446, 16558, +, 1113, 135079; cds-WP_000809168.1, NZ_CP025268.1, 16752, 16904, -, 153, 418; cds-WP_000681354.1, NZ_CP025268.1, 17490, 18656, +, 1167, 112; cds-WP_001338226.1, NZ_CP025268.1, 18722, 19621, +, 900, 10211; cds-CXP41_RS24330, NZ_CP025268.1, 19659, 19796, -, 138, 5; cds-CXP41_RS00100, NZ_CP025268.1, 19812, 20509, -, 698, 156287; cds-WP_001274021.1, NZ_CP025268.1, 20816, 21079, -, 264, 43781; cds-WP_001300728.1, NZ_CP025268.1, 21182, 21400, +, 219, 2173; cds-WP_000767329.1, NZ_CP025268.1, 21408, 22349, +, 942, 252327; cds-WP_001286857.1, NZ_CP025268.1, 22392, 25208, +, 2817, 1999037; cds-WP_000083372.1, NZ_CP025268.1, 25208, 25702, +, 495, 290863; cds-WP_000004655.1, NZ_CP025268.1, 25827, 26276, +, 450, 597564; cds-WP_001166395.1, NZ_CP025268.1, 26278, 27228, +, 951, 334110; cds-WP_001239142.1, NZ_CP025268.1, 27294, 28208, +, 915, 33875; cds-WP_000543604.1, NZ_CP025268.1, 28375, 29196, +, 822, 48722; cds-WP_000597260.1, NZ_CP025268.1, 29652, 30800, +, 1149, 263909; cds-WP_001126348.1,
NZ_CP025268.1, 30818, 34039, +, 3222, 299328; cds-WP_000333125.1, NZ_CP025268.1, 34301, 34696, +, 396, 208; cds-WP_000122871.1, NZ_CP025268.1, 34782, 35372, -, 591, 0; cds-WP_001295419.1, NZ_CP025268.1, 35378, 36163, -, 786, 0; cds-WP_001350478.1, NZ_CP025268.1, 36272, 37825, -, 1554, 8947; cds-WP_101348527.1, NZ_CP025268.1, 37899, 39116, -, 1218, 3; cds-WP_000347117.1, NZ_CP025268.1, 39245, 40387, -, 1143, 0; cds-WP_000787103.1, NZ_CP025268.1, 40418, 41932, -, 1515, 0; cds-WP_000692204.1, NZ_CP025268.1, 42404, 43174, +, 771, 0; cds-WP_001091499.1, NZ_CP025268.1, 43189, 44130, +, 942, 0; cds-WP_001287715.1, NZ_CP025268.1, 44181, 45467, +, 1287, 0; cds-WP_000203741.1, NZ_CP025268.1, 45464, 45751, +, 288, 0; cds-WP_001183198.1, NZ_CP025268.1, 45808, 47139, +, 1332, 0; cds-WP_000600725.1, NZ_CP025268.1, 47247, 47777, +, 531, 21; cds-WP_101348528.1, NZ_CP025268.1, 47770, 49632, +, 1863, 0; cds-WP_000624375.1, NZ_CP025268.1, 49824, 50303, +, 480, 2; cds-WP_000257192.1, NZ_CP025268.1, 50381, 51223, -, 843, 88424; cds-WP_000610901.1, NZ_CP025268.1, 51230, 51607, -, 378, 119806; cds-WP_001065381.1, NZ_CP025268.1, 51610, 52431, -, 822, 79948; cds-WP_000241277.1, NZ_CP025268.1, 52428, 53417, -, 990, 784424; cds-WP_000800457.1, NZ_CP025268.1, 53417, 54703, -, 1287, 998930; cds-WP_000746151.1, NZ_CP025268.1, 54756, 57110, -, 2355, 816469; cds-WP_001200579.1, NZ_CP025268.1, 57365, 58180, +, 816, 24699; cds-WP_001393555.1, NZ_CP025268.1, 58475, 59125, +, 651, 39794; cds-WP_001393554.1, NZ_CP025268.1, 59122, 59280, +, 159, 45; cds-CXP41_RS00280, NZ_CP025268.1, 59297, 59475, -, 179, 28; cds-WP_000525176.1, NZ_CP025268.1, 59688, 60347, -, 660, 101855; cds-WP_001117011.1, NZ_CP025268.1, 60359, 63265, -, 2907, 1560983; cds-WP_000035670.1, NZ_CP025268.1, 63430, 65781, -, 2352, 70731; cds-WP_101348529.1, NZ_CP025268.1, 65856, 66551, -, 696, 0; cds-WP_000151748.1, NZ_CP025268.1, 66666, 68168, -, 1503, 87024; cds-WP_000951856.1, NZ_CP025268.1, 68179, 69879, -, 1701, 0; cds-WP_001300811.1, NZ_CP025268.1, 70218, 71096, +, 879, 0; cds-WP_001148390.1, NZ_CP025268.1, 71182, 71946, +, 765, 1319; cds-WP_000916281.1, NZ_CP025268.1, 72060, 72758, -, 699, 4996; cds-WP_000235721.1, NZ_CP025268.1, 72742, 74352, -, 1611, 4943; cds-WP_101348530.1, NZ_CP025268.1, 74328, 75311, -, 984, 105916; cds-WP_001138624.1, NZ_CP025268.1, 75475, 77130, -, 1656, 560025; cds-WP_001248770.1, NZ_CP025268.1, 77219, 77350, +, 132, 0; cds-WP_000637847.1, NZ_CP025268.1, 77452, 78630, +, 1179, 1; cds-WP_000818228.1, NZ_CP025268.1, 78679, 79284, -, 606, 37641; cds-WP_001140652.1, NZ_CP025268.1, 79295, 80695, -, 1401, 12487; cds-WP_000042353.1, NZ_CP025268.1, 80698, 81789, -, 1092, 74683; cds-WP_000082850.1, NZ_CP025268.1, 81789, 83360, -, 1572, 0; cds-WP_001300467.1, NZ_CP025268.1, 83453, 83539, -, 87, 0; cds-WP_001115472.1, NZ_CP025268.1, 84199, 85143, +, 945, 0; cds-WP_120795371.1, NZ_CP025268.1, 85298, 85342, -, 45, 0; cds-WP_001295534.1, NZ_CP025268.1, 85461, 87185, +, 1725, 7; cds-WP_001300383.1, NZ_CP025268.1, 87188, 87679, +, 492, 1; cds-WP_001303787.1, NZ_CP025268.1, 87691, 87777, +, 87, 2; cds-WP_000762401.1, NZ_CP025268.1, 87859, 88863, +, 1005, 214052; cds-WP_001295533.1, NZ_CP025268.1, 89465, 89923, +, 459, 4804; cds-WP_000970479.1, NZ_CP025268.1, 89925, 90866, +, 942, 473538; cds-WP_000625658.1, NZ_CP025268.1, 90863, 91228, +, 366, 221353; cds-WP_000642196.1, NZ_CP025268.1, 91244, 93010, +, 1767, 696601; cds-WP_000775093.1, NZ_CP025268.1, 92997, 94484, +, 1488, 362751; cds-WP_000626685.1, NZ_CP025268.1, 94481, 95839, +, 1359, 719053; cds-WP_101348531.1, NZ_CP025268.1, 95833, 96915, +, 1083, 223379; cds-WP_000796481.1, NZ_CP025268.1, 96918, 98234, +, 1317, 61584; cds-WP_001295532.1, NZ_CP025268.1, 98234, 99478, +, 1245, 240164; cds-WP_000016560.1, NZ_CP025268.1, 99475, 100542, +, 1068, 137396; cds-WP_001096049.1, NZ_CP025268.1, 100596, 102071, +, 1476, 966539; cds-WP_000130056.1, NZ_CP025268.1, 102064, 102984, +, 921, 454721; cds-WP_000075748.1, NZ_CP025268.1, 102986, 103816, +, 831, 72573; cds-WP_101348532.1, NZ_CP025268.1, 103813, 105075, +, 1263, 895413; cds-WP_000462776.1, NZ_CP025268.1, 105136, 106287, +, 1152, 268973; cds-WP_000595482.1, NZ_CP025268.1, 106388, 107305, +, 918, 637582; cds-WP_000014321.1, NZ_CP025268.1, 107536, 108048, +, 513, 183489; cds-WP_000905789.1, NZ_CP025268.1, 108110, 110815, +, 2706, 1485344; cds-WP_000736007.1, NZ_CP025268.1, 110875, 111264, +, 390, 3454; cds-WP_000005042.1, NZ_CP025268.1, 111480, 111677, -, 198, 289; cds-WP_101348533.1, NZ_CP025268.1, 111687, 112430, -, 744, 9140; cds-WP_001269520.1, NZ_CP025268.1, 112430, 113050, -, 621, 18240; cds-WP_120795372.1, NZ_CP025268.1, 113075, 113119, +, 45, 0; cds-WP_001217338.1, NZ_CP025268.1, 113275, 114318, +, 1044, 18118; cds-WP_101348534.1, NZ_CP025268.1, 114353, 115555, -, 1203, 0; cds-WP_001025145.1, NZ_CP025268.1, 115545, 116930, -, 1386, 8357; cds-WP_000360904.1, NZ_CP025268.1, 116940, 117380, -, 441, 0; cds-WP_101348535.1, NZ_CP025268.1, 117583, 118476, -, 894, 0; cds-WP_000923721.1, NZ_CP025268.1, 118564, 119115, +, 552, 35; cds-WP_000172005.1, NZ_CP025268.1, 119112, 119966, +, 855, 10; cds-WP_000969915.1, NZ_CP025268.1, 120009, 121382, -, 1374, 1439; cds-WP_000331776.1, NZ_CP025268.1, 121923, 122687, +, 765, 872024; cds-WP_000003820.1, NZ_CP025268.1, 122848, 125511, +, 2664, 17016091; cds-WP_000963518.1, NZ_CP025268.1, 125526, 127418, +, 1893, 9504704; cds-WP_000102485.1, NZ_CP025268.1, 127743, 129167, +, 1425, 3914037; cds-WP_000784470.1, NZ_CP025268.1, 129238, 131091, -, 1854, 28; cds-WP_001307570.1, NZ_CP025268.1, 131446, 134043, +, 2598, 1131892; cds-WP_000384306.1, NZ_CP025268.1, 134219, 134581, +, 363, 4701; cds-WP_000734287.1, NZ_CP025268.1, 134619, 135413, -, 795, 125317; cds-WP_000818411.1, NZ_CP025268.1, 135429, 136295, -, 867, 147739; cds-WP_001295568.1, NZ_CP025268.1, 136401, 136748, -, 348, 3; cds-WP_101348536.1, NZ_CP025268.1, 136914, 138464, +, 1551, 27; cds-WP_001306211.1, NZ_CP025268.1, 138666, 141056, -, 2391, 592226; cds-WP_000683335.1, NZ_CP025268.1, 141262, 141798, +, 537, 104861; cds-WP_000651599.1, NZ_CP025268.1, 141839, 142501, -, 663, 316787; cds-WP_000150637.1, NZ_CP025268.1, 142610, 143536, +, 927, 82309; cds-WP_000972203.1, NZ_CP025268.1, 143533, 144303, +, 771, 87583; cds-WP_000901987.1, NZ_CP025268.1, 144408, 144848, +, 441, 4980; cds-WP_000277917.1, NZ_CP025268.1, 144912, 146141, +, 1230, 11516; cds-WP_000621515.1, NZ_CP025268.1, 146145, 146525, -, 381, 8; cds-WP_000339954.1, NZ_CP025268.1, 146799, 147701, +, 903, 11178; cds-WP_000905383.1, NZ_CP025268.1, 147775, 148626, -, 852, 52229; cds-WP_000805497.1, NZ_CP025268.1, 148638, 149432, -, 795, 41065; cds-WP_101348537.1, NZ_CP025268.1, 149546, 150784, -, 1239, 0; cds-WP_000553749.1, NZ_CP025268.1, 150834, 151430, -, 597, 0; cds-WP_101348538.1, NZ_CP025268.1, 151457, 152062, -, 606, 0; cds-WP_000598300.1, NZ_CP025268.1, 152074, 152643, -, 570, 0; cds-WP_000151605.1, NZ_CP025268.1, 152660, 155257, -, 2598, 0; cds-WP_000465928.1, NZ_CP025268.1, 155292, 156032, -, 741, 0; cds-WP_000038438.1, NZ_CP025268.1, 156130, 156714, -, 585, 150; cds-WP_000215139.1, NZ_CP025268.1, 157084, 157563, -, 480, 82507; cds-WP_010723084.1, NZ_CP025268.1, 157560, 158957, -, 1398, 812071; cds-WP_000937424.1, NZ_CP025268.1, 159017, 159943, -, 927, 98409; cds-WP_001155227.1, NZ_CP025268.1, 159980, 160435, -, 456, 4116; cds-WP_000396036.1, NZ_CP025268.1, 160613, 161317, -, 705, 28044; cds-WP_001294700.1, NZ_CP025268.1, 161332, 161862, -, 531, 14833; cds-WP_101348539.1, NZ_CP025268.1, 161936, 164365, +, 2430, 46703; cds-WP_000918162.1, NZ_CP025268.1, 164561, 167095, +, 2535, 36024; cds-WP_101348540.1, NZ_CP025268.1, 167315, 169558, +, 2244, 215038; cds-WP_001158931.1, NZ_CP025268.1, 169609, 170406, +, 798, 7752; cds-WP_001310529.1, NZ_CP025268.1, 170406, 171296, +, 891, 12; cds-WP_000044072.1, NZ_CP025268.1, 171293, 173275, +, 1983, 382956; cds-WP_000045315.1, NZ_CP025268.1, 173433, 174713, -, 1281, 151714; cds-WP_211180485.1, NZ_CP025268.1, 174879, 174926, +, 48, 2351; cds-WP_000845394.1, NZ_CP025268.1, 174938, 176359, +, 1422, 465366; cds-WP_120795373.1, NZ_CP025268.1, 176383, 176448, +, 66, 1051; cds-WP_001295564.1, NZ_CP025268.1, 176441, 176785, +, 345, 7116; cds-WP_000964221.1, NZ_CP025268.1, 176832, 177455, -, 624, 144; cds-WP_001129927.1, NZ_CP025268.1, 177493, 178293, -, 801, 15430; cds-WP_000689844.1, NZ_CP025268.1, 178286, 178984, -, 699, 20; cds-WP_000057073.1, NZ_CP025268.1, 179068, 180585, +, 1518, 19959; cds-WP_000753946.1, NZ_CP025268.1, 180715, 182139, +, 1425, 142768; cds-CXP41_RS00845, NZ_CP025268.1, 182294, 183436, +, 1143, 79617; cds-WP_000272188.1, NZ_CP025268.1, 183539, 183925, -, 387, 59; cds-WP_000625631.1, NZ_CP025268.1, 184087, 184899, -, 813, 892; cds-WP_001186650.1, NZ_CP025268.1, 184953, 185777, -, 825, 553; cds-WP_001094586.1, NZ_CP025268.1, 185808, 188480, -, 2673, 296534; cds-WP_001018194.1, NZ_CP025268.1, 188542, 189336, -, 795, 311102; cds-WP_000246882.1, NZ_CP025268.1, 189704, 190429, +, 726, 6151197; cds-WP_000818114.1, NZ_CP025268.1, 190687, 191538, +, 852, 1892216; cds-WP_000224573.1, NZ_CP025268.1, 191685, 192410, +, 726, 1521610; cds-WP_000622418.1, NZ_CP025268.1, 192702, 193259, +, 558, 830799; cds-WP_000811923.1, NZ_CP025268.1, 193351, 194547, +, 1197, 65026; cds-WP_001295562.1, NZ_CP025268.1, 194736, 195494, +, 759, 199767; cds-WP_000922446.1, NZ_CP025268.1, 195507, 196364, +, 858, 648915; cds-WP_001295561.1, NZ_CP025268.1, 196376, 197728, +, 1353, 1295660; cds-WP_101348541.1, NZ_CP025268.1, 197758, 200190, +, 2433, 2246169; cds-WP_000758956.1, NZ_CP025268.1, 200312, 200797, +, 486, 430588; cds-WP_001139279.1, NZ_CP025268.1, 200801, 201826, +, 1026, 726837; cds-WP_000210739.1, NZ_CP025268.1, 201931, 202386, +, 456, 519088; cds-WP_000565966.1, NZ_CP025268.1, 202390, 203178, +, 789, 177761; cds-WP_000139654.1, NZ_CP025268.1, 203178, 204326, +, 1149, 602474; cds-WP_000569430.1, NZ_CP025268.1, 204323, 204919, +, 597, 237509; cds-WP_001294757.1, NZ_CP025268.1, 204956, 208438, +, 3483, 1586443; cds-WP_000055741.1, NZ_CP025268.1, 208451, 209410, +, 960, 809920; cds-WP_001020973.1, NZ_CP025268.1, 209509, 211650, +, 2142, 118208; cds-WP_000901099.1, NZ_CP025268.1, 211707, 212096, +, 390, 226829; cds-WP_000176549.1, NZ_CP025268.1, 212161, 213459, +, 1299, 193; cds-WP_000062312.1, NZ_CP025268.1, 213508, 213768, -, 261, 3; cds-WP_000417058.1, NZ_CP025268.1, 213755, 213955, -, 201, 23; cds-WP_001185290.1, NZ_CP025268.1, 214121, 214666, +, 546, 9668; cds-WP_000635537.1, NZ_CP025268.1, 214663, 215085, +, 423, 89619; cds-WP_000239163.1, NZ_CP025268.1, 215099, 215809, +, 711, 218; cds-WP_001336393.1, NZ_CP025268.1, 216009, 216833, -, 825, 816; cds-WP_001260717.1, NZ_CP025268.1, 216887, 218605, -, 1719, 469255; cds-WP_000094011.1, NZ_CP025268.1, 218717, 219424, -, 708, 74005; cds-WP_001202329.1, NZ_CP025268.1, 219421, 219825, -, 405, 95030; cds-WP_000874226.1, NZ_CP025268.1, 219943, 220758, -, 816, 34349; cds-WP_001294600.1, NZ_CP025268.1, 220798, 221451, -, 654, 17583; cds-WP_000594006.1, NZ_CP025268.1, 221444, 222475, -, 1032, 348221; cds-WP_001140187.1, NZ_CP025268.1, 222663, 223238, +, 576, 12; cds-WP_000997010.1, NZ_CP025268.1, 228997, 229800, +, 804, 211220; cds-WP_000648572.1, NZ_CP025268.1, 229797, 230711, -,915, 134097; cds-WP_001230983.1, NZ_CP025268.1, 230952, 231752, +, 801, 1613; cds-CXP41_RS01095, NZ_CP025268.1, 231783, 232379, +, 597, 0; cds-WP_000644685.1, NZ_CP025268.1, 232427, 233785, -, 1359, 64163; cds-WP_001052715.1, NZ_CP025268.1, 233857, 234612, -, 756, 4484; cds-WP_001326702.1, NZ_CP025268.1, 234646, 235368, +, 723, 1492; cds-WP_000917883.1, NZ_CP025268.1, 235365, 235832, -, 468, 74891; cds-WP_001340895.1, NZ_CP025268.1, 235897, 236628, +, 732, 10; cds-WP_001086141.1, NZ_CP025268.1, 237165, 237950, +, 786, 27; cds-WP_001236645.1, NZ_CP025268.1, 238087, 238566, -, 480, 411; cds-CXP41_RS01145, NZ_CP025268.1, 238576, 238932, -, 357, 0; cds-CXP41_RS01150, NZ_CP025268.1, 238930, 239208, +, 279, 95; cds-WP_001118036.1, NZ_CP025268.1, 239249, 240019, -, 771, 10; cds-WP_001308392.1, NZ_CP025268.1, 240200, 240646, +, 447, 1; cds-WP_000973083.1, NZ_CP025268.1, 240689, 243133, -, 2445, 264; cds-WP_000284050.1, NZ_CP025268.1, 243373, 243951, +, 579, 40658; cds-WP_000333380.1, NZ_CP025268.1, 244157, 244924, +, 768, 57063; cds-WP_001225679.1, NZ_CP025268.1, 244895, 245635, -, 741, 21211; cds-WP_000615983.1,
NZ_CP025268.1, 245791, 246069, -, 279, 0; cds-WP_000729703.1, NZ_CP025268.1, 246072, 246332, -, 261, 0; cds-WP_000056849.1, NZ_CP025268.1, 246542, 247291, +, 750, 0; cds-WP_000006255.1, NZ_CP025268.1, 247466, 247963, +, 498, 93; cds-CXP41_RS01205, NZ_CP025268.1, 248187, 249899, -, 1713, 0; cds-WP_000207552.1, NZ_CP025268.1, 249871, 250656, +, 786, 0; cds-WP_001226164.1, NZ_CP025268.1, 250727, 251782, +, 1056, 0; cds-WP_000554758.1, NZ_CP025268.1, 251834, 252127, +, 294, 571; cds-WP_001263489.1, NZ_CP025268.1, 252130, 252528, +, 399, 863; cds-WP_101348542.1, NZ_CP025268.1, 252538, 252990, +, 453, 33; cds-CXP41_RS01235, NZ_CP025268.1, 253308, 253514, +, 207, 0; cds-CXP41_RS01240, NZ_CP025268.1, 253510, 254031, +, 522, 3437; cds-WP_101348543.1, NZ_CP025268.1, 254088, 255545, -, 1458, 26869; cds-WP_001291990.1, NZ_CP025268.1, 255806, 256264, +, 459, 33035; cds-WP_000189532.1, NZ_CP025268.1, 256356, 257600, +, 1245, 117129; cds-WP_000174689.1, NZ_CP025268.1, 257658, 258059, +, 402, 7439; cds-WP_000749863.1, NZ_CP025268.1, 258098, 259153, -, 1056, 8420; cds-WP_001285288.1, NZ_CP025268.1, 259441, 260544, +, 1104, 191443; cds-WP_094314171.1, NZ_CP025268.1, 260556, 261809, +, 1254, 275458; cds-WP_000854672.1, NZ_CP025268.1, 262381, 262722, -, 342, 5804; cds-WP_000070395.1, NZ_CP025268.1, 262743, 263060, -, 318, 50; cds-WP_000691994.1, NZ_CP025268.1, 263079, 263300, -, 222, 143; cds-WP_000811693.1, NZ_CP025268.1, 263309, 263785, -, 477, 368; cds-WP_000211838.1, NZ_CP025268.1, 263801, 264259, -, 459, 763; cds-WP_000194654.1, NZ_CP025268.1, 264357, 264596, -, 240, 0; cds-WP_001547765.1, NZ_CP025268.1, 264673, 265140, -, 468, 0; cds-WP_000824223.1, NZ_CP025268.1, 265163, 265606, -, 444, 2361; cds-WP_001548158.1, NZ_CP025268.1, 265606, 265833, -, 228, 0; cds-CXP41_RS01330, NZ_CP025268.1, 265829, 266020, -, 192, 0; cds-WP_000197389.1, NZ_CP025268.1, 266237, 267058, -, 822, 0; cds-WP_001065553.1, NZ_CP025268.1, 267150, 268013, -, 864, 0; cds-WP_000770246.1, NZ_CP025268.1, 268342, 269235, -, 894, 832; cds-CXP41_RS01355, NZ_CP025268.1, 269331, 269588, +, 258, 6; cds-WP_001254938.1, NZ_CP025268.1, 269656, 270807, +, 1152, 902; cds-CXP41_RS01365, NZ_CP025268.1, 270817, 271242, +, 426, 50185; cds-CXP41_RS01375, NZ_CP025268.1, 271915, 273006, +, 1092, 72762; cds-WP_000019403.1, NZ_CP025268.1, 273154, 274134, -, 981, 191221; cds-WP_001393629.1, NZ_CP025268.1, 274378, 275781, +, 1404, 0; cds-WP_000081352.1, NZ_CP025268.1, 275768, 276700, +, 933, 2; cds-WP_000192349.1, NZ_CP025268.1, 276809, 277855, -, 1047, 0; cds-CXP41_RS01405, NZ_CP025268.1, 277867, 278214, -, 348, 0; cds-CXP41_RS01410, NZ_CP025268.1, 278231, 278928, -, 698, 21896; cds-CXP41_RS01420, NZ_CP025268.1, 278984, 279157, +, 174, 3; cds-CXP41_RS01425, NZ_CP025268.1, 279167, 279415, -, 249, 0; cds-WP_001030800.1, NZ_CP025268.1, 279438, 279788, -, 351, 0; cds-CXP41_RS01435, NZ_CP025268.1, 279882, 281035, -, 1154, 0; cds-WP_001136613.1, NZ_CP025268.1, 281330, 282238, +, 909, 511; cds-WP_101348544.1, NZ_CP025268.1, 282253, 284220, +, 1968, 1; cds-WP_000192863.1, NZ_CP025268.1, 284447, 285829, +, 1383, 4; cds-WP_101348545.1, NZ_CP025268.1, 285841, 287451, +, 1611, 64081; cds-WP_001121657.1, NZ_CP025268.1, 287456, 288214, -, 759, 11474; cds-WP_001281825.1, NZ_CP025268.1, 288353, 289357, -, 1005, 0; cds-WP_211180486.1, NZ_CP025268.1, 289327, 289422, -, 96, 0; cds-CXP41_RS01470, NZ_CP025268.1, 289481, 289684, +, 204, 0; cds-CXP41_RS01480, NZ_CP025268.1, 289701, 290398, -, 698, 20405; cds-CXP41_RS01490, NZ_CP025268.1, 290453, 291283, +, 831, 715; cds-WP_000860996.1, NZ_CP025268.1, 291374, 292000, -, 627, 0; cds-WP_001214248.1, NZ_CP025268.1, 292272, 292970, -, 699, 1; cds-WP_000072039.1, NZ_CP025268.1, 292997, 293851, -, 855, 0; cds-WP_001272156.1, NZ_CP025268.1, 293970, 294194, -, 225, 0; cds-WP_000224818.1, NZ_CP025268.1, 294191, 294631, -, 441, 78; cds-WP_001407714.1, NZ_CP025268.1, 294748, 296148, -, 1401, 125; cds-CXP41_RS01530, NZ_CP025268.1, 296433, 296771, -, 339, 14; cds-WP_000121359.1, NZ_CP025268.1, 296822, 297778, -, 957, 38; cds-WP_000667026.1, NZ_CP025268.1, 297788, 299986, -, 2199, 0; cds-WP_000643333.1, NZ_CP025268.1, 299983, 300939, -, 957, 0; cds-WP_000070700.1, NZ_CP025268.1, 300936, 301625, -, 690, 0; cds-WP_001019920.1, NZ_CP025268.1, 302043, 302657, +, 615, 1135; cds-WP_001303809.1, NZ_CP025268.1, 302905, 303234, -, 330, 0; cds-WP_001350485.1, NZ_CP025268.1, 303547, 304257, -, 711, 0; cds-WP_001310578.1, NZ_CP025268.1, 304226, 305869, -, 1644, 23; cds-WP_001131096.1, NZ_CP025268.1, 305859, 308384, -, 2526, 1901; cds-WP_000716398.1, NZ_CP025268.1, 308410, 309078, -, 669, 0; cds-WP_000730972.1, NZ_CP025268.1, 309136, 309723, -, 588, 0; cds-WP_001301257.1, NZ_CP025268.1, 309798, 310340, -, 543, 0; cds-WP_001147279.1, NZ_CP025268.1, 311164, 311391, +, 228, 0; cds-WP_000866436.1, NZ_CP025268.1, 311426, 311566, -, 141, 0; cds-WP_000803998.1, NZ_CP025268.1, 311566, 311829, -, 264, 0; cds-WP_000662258.1, NZ_CP025268.1, 312193, 312294, -, 102, 0; cds-CXP41_RS01640, NZ_CP025268.1, 313409, 314284, +, 876, 0; cds-WP_085947771.1, NZ_CP025268.1;NZ_CP025268.1, 314343;314604, 314604;315505, +;+, 1163, 4; cds-CXP41_RS01650, NZ_CP025268.1, 315535, 316188, -, 654, 79341; cds-CXP41_RS24070, NZ_CP025268.1, 316491, 316619, +, 129, 0; cds-WP_001299021.1, NZ_CP025268.1, 316778, 317371, -, 594, 250; cds-WP_000474084.1, NZ_CP025268.1, 317383, 317619, -, 237, 0; cds-WP_001046307.1, NZ_CP025268.1, 317728, 319053, -, 1326, 0; cds-WP_000339594.1, NZ_CP025268.1, 319279, 320133, +, 855, 0; cds-WP_001102108.1, NZ_CP025268.1, 320659, 321378, +, 720, 1; cds-WP_032289499.1, NZ_CP025268.1, 321389, 322816, +, 1428, 20174; cds-WP_000370307.1, NZ_CP025268.1, 322809, 323504, +, 696, 0; cds-WP_001209100.1, NZ_CP025268.1, 323747, 324415, -, 669, 814; cds-WP_001159094.1, NZ_CP025268.1, 324628, 326298, -, 1671, 205497; cds-WP_000089110.1, NZ_CP025268.1, 326312, 327784, -, 1473, 92537; cds-WP_001335745.1, NZ_CP025268.1, 327798, 328385, -, 588, 9761; cds-WP_000131044.1, NZ_CP025268.1, 328514, 330547, +, 2034, 318484; cds-WP_120795374.1, NZ_CP025268.1, 330853, 330927, +, 75, 0; cds-WP_001301264.1, NZ_CP025268.1, 331422, 332510, +, 1089, 20559; cds-WP_001084394.1, NZ_CP025268.1, 332552, 333484, -, 933, 9; cds-WP_001013892.1, NZ_CP025268.1, 333576, 334073, -, 498, 6; cds-WP_000023635.1, NZ_CP025268.1, 334331, 334936, +, 606, 0; cds-CXP41_RS01765, NZ_CP025268.1, 334976, 335839, +, 864, 0; cds-WP_000111836.1, NZ_CP025268.1, 335829, 337376, +, 1548, 0; cds-WP_000083429.1, NZ_CP025268.1, 337376, 338794, +, 1419, 0; cds-WP_001295687.1, NZ_CP025268.1, 338844, 339140, +, 297, 7; cds-WP_000661672.1, NZ_CP025268.1, 339216, 340166, +, 951, 0; cds-WP_000665120.1, NZ_CP025268.1, 340176, 341558, +, 1383, 0; cds-WP_000692754.1, NZ_CP025268.1, 341935, 342984, +, 1050, 151092; cds-WP_001381673.1, NZ_CP025268.1, 343227, 344042, +, 816, 0; cds-WP_000290616.1, NZ_CP025268.1, 344455, 344700, +, 246, 0; cds-WP_000983410.1, NZ_CP025268.1, 344717, 345388, -, 672, 0; cds-WP_000691956.1, NZ_CP025268.1, 345535, 345810, +, 276, 0; cds-WP_000941041.1, NZ_CP025268.1, 345908, 347494, -, 1587, 0; cds-WP_000052206.1, NZ_CP025268.1, 347733, 348623, +, 891, 2; cds-WP_001285927.1, NZ_CP025268.1, 349063, 350232, +, 1170, 0; cds-WP_001275859.1, NZ_CP025268.1, 350266, 351717, +, 1452, 0; cds-WP_000010288.1, NZ_CP025268.1, 351757, 353643, +, 1887, 0; cds-WP_000076233.1, NZ_CP025268.1, 353973, 355232, +, 1260, 125307; cds-WP_001301240.1, NZ_CP025268.1, 355222, 356505, +, 1284, 8118; cds-WP_000952503.1, NZ_CP025268.1, 356842, 357741, -, 900, 0; cds-WP_000658652.1, NZ_CP025268.1, 357850, 358509, +, 660, 2; cds-WP_000616243.1, NZ_CP025268.1, 358540, 359010, +, 471, 0; cds-WP_001301263.1, NZ_CP025268.1, 359016, 360197, +, 1182, 0; cds-WP_001335915.1, NZ_CP025268.1, 360300, 360911, -, 612, 0; cds-WP_000291549.1, NZ_CP025268.1, 360977, 362230, -, 1254, 0; cds-WP_000177906.1, NZ_CP025268.1, 362282, 365356, -, 3075, 2; cds-WP_000805902.1, NZ_CP025268.1, 365479, 366561, -, 1083, 302; cds-WP_001301325.1, NZ_CP025268.1, 366638, 367471, -, 834, 0; cds-WP_001007407.1, NZ_CP025268.1, 367662, 369326, +, 1665, 0; cds-WP_000543457.1, NZ_CP025268.1, 369328, 370272, +, 945, 0; cds-CXP41_RS01915, NZ_CP025268.1, 370290, 371156, +, 867, 0; cds-WP_000160710.1, NZ_CP025268.1, 371166, 371975, +, 810, 0; cds-WP_101348546.1, NZ_CP025268.1, 371972, 372922, +, 951, 0; cds-WP_001013499.1, NZ_CP025268.1, 372919, 373932, +, 1014, 0; cds-WP_000107627.1, NZ_CP025268.1, 374510, 375721, +, 1212, 3; cds-WP_001096705.1, NZ_CP025268.1, 375823, 376362, +, 540, 0; cds-WP_000419081.1, NZ_CP025268.1, 376586, 377419, -, 834, 92829; cds-WP_000842106.1, NZ_CP025268.1, 377513, 378622, -, 1110, 136110; cds-WP_001141271.1, NZ_CP025268.1, 378657, 378932, -, 276, 1176; cds-WP_000596084.1, NZ_CP025268.1, 379120, 379893, -, 774, 0; cds-WP_000012218.1, NZ_CP025268.1, 379895, 380338, -, 444, 467; cds-WP_088895425.1, NZ_CP025268.1;NZ_CP025268.1, 380402;380717, 380717;381630, +;+, 1229, 8565; cds-CXP41_RS01980, NZ_CP025268.1, 381639, 381824, -, 186, 0; cds-WP_101348547.1, NZ_CP025268.1, 381790, 382986, -, 1197, 0; cds-CXP41_RS01990, NZ_CP025268.1, 382996, 383667, -, 672, 0; cds-WP_101348548.1, NZ_CP025268.1, 384283, 385245, +, 963, 0; cds-WP_000939375.1, NZ_CP025268.1, 385258, 386025, +, 768, 0; cds-WP_000114620.1, NZ_CP025268.1, 386022, 386849, +, 828, 0; cds-WP_000004027.1, NZ_CP025268.1, 386846, 387697, +, 852, 8290; cds-WP_001295337.1, NZ_CP025268.1, 387804, 388778, -, 975, 519371; cds-CXP41_RS02030, NZ_CP025268.1, 389302, 390828, +, 1527, 10331; cds-WP_101348549.1, NZ_CP025268.1;NZ_CP025268.1, 390790;391691, 391691;391952, -;-, 1163, 5; cds-CXP41_RS02040, NZ_CP025268.1, 392015, 393469, +, 1455, 243; cds-WP_001295335.1, NZ_CP025268.1, 393557, 394180, +, 624, 0; cds-WP_000830741.1, NZ_CP025268.1, 394181, 395338, -, 1158, 0; cds-WP_001301663.1, NZ_CP025268.1, 395690, 396910, +, 1221, 208421; cds-WP_000092067.1, NZ_CP025268.1, 396923, 398017, +, 1095, 555; cds-WP_000763151.1, NZ_CP025268.1, 398076, 398384, -, 309, 0; cds-WP_001295334.1, NZ_CP025268.1, 398644, 398856, +, 213, 7292; cds-WP_000413677.1, NZ_CP025268.1, 398880, 399974, -, 1095, 138485; cds-WP_001300163.1, NZ_CP025268.1, 400052, 400255, +, 204, 0; cds-CXP41_RS02095, NZ_CP025268.1, 400437, 400696, +, 260, 0; cds-WP_000814403.1, NZ_CP025268.1, 400797, 402212, +, 1416, 0; cds-WP_001295332.1, NZ_CP025268.1, 402331, 402651, +, 321, 0; cds-WP_000484048.1, NZ_CP025268.1, 402753, 403868, +, 1116, 4; cds-WP_001295331.1, NZ_CP025268.1, 403885, 404694, -, 810, 0; cds-WP_000158159.1, NZ_CP025268.1, 404814, 405272, +, 459, 1704; cds-WP_000193393.1, NZ_CP025268.1, 405455, 405979, +, 525, 274152; cds-WP_001142439.1, NZ_CP025268.1, 406029, 406220, +, 192, 136107; cds-WP_001276420.1, NZ_CP025268.1, 406478, 407155, +, 678, 51080; cds-WP_000941942.1, NZ_CP025268.1, 407227, 407511, +, 285, 5361; cds-CXP41_RS02155, NZ_CP025268.1, 407659, 408000, +, 342, 110696; cds-WP_120795376.1, NZ_CP025268.1, 407997, 408080, +, 84, 45022; cds-WP_001298537.1, NZ_CP025268.1, 408158, 409069, -, 912, 364132; cds-WP_001219309.1, NZ_CP025268.1, 409194, 410102, +, 909, 43; cds-WP_001326926.1, NZ_CP025268.1, 410347, 411531, -, 1185, 0; cds-WP_000698951.1, NZ_CP025268.1, 411657, 414803, -, 3147, 20025; cds-WP_001221319.1, NZ_CP025268.1, 414800, 416002, -, 1203, 18989; cds-WP_000113933.1, NZ_CP025268.1, 416192, 416881, +, 690, 19; cds-WP_000893623.1, NZ_CP025268.1, 416939, 418234, +, 1296, 3; cds-WP_000149639.1, NZ_CP025268.1, 418641, 419960, +, 1320, 221773; cds-WP_001295329.1, NZ_CP025268.1, 420036, 421409, +, 1374, 192918; cds-WP_001310597.1, NZ_CP025268.1, 421565, 423382, +, 1818, 70733; cds-WP_001009885.1, NZ_CP025268.1, 423387, 423968, -, 582, 0; cds-WP_001266503.1, NZ_CP025268.1, 424061, 425131, +, 1071, 36189; cds-WP_000667319.1, NZ_CP025268.1, 425187, 426314, +, 1128, 219819; cds-WP_000007629.1, NZ_CP025268.1, 426337, 426669, +, 333, 297141; cds-WP_000934822.1, NZ_CP025268.1, 426697, 428544, +, 1848, 1875237; cds-WP_000046637.1, NZ_CP025268.1, 428555, 429526, +, 972, 467087; cds-WP_000974813.1, NZ_CP025268.1, 429655, 430002, +, 348, 38; cds-WP_001295328.1, NZ_CP025268.1, 430179, 431063, -, 885, 99222; cds-WP _001326929.1, NZ_CP025268.1, 431362, 431901, -, 540, 0; cds-WP_000543535.1, NZ_CP025268.1, 432052, 432501, +, 450, 120; cds-WP_001150457.1, NZ_CP025268.1, 432505, 433608, +, 1104, 581; cds-WP_001021161.1, NZ_CP025268.1, 433697, 434167, +, 471, 251662; cds-WP_000801125.1,
NZ_CP025268.1, 434187, 434606, +, 420, 179709; cds-WP_000742109.1, NZ_CP025268.1, 434684, 435661, +, 978, 191140; cds-WP_000154044.1, NZ_CP025268.1, 435639, 436157, +, 519, 128798; cds-WP_001199800.1, NZ_CP025268.1, 436211, 437185, -, 975, 79281; cds-WP_000006797.1, NZ_CP025268.1, 437365, 439227, -, 1863, 482472; cds-WP_000347217.1, NZ_CP025268.1, 439252, 440151, -, 900, 277058; cds-WP_001124935.1, NZ_CP025268.1, 440151, 440393, -, 243, 9897; cds-WP_000668662.1, NZ_CP025268.1, 440599, 442047, +, 1449, 620556; cds-WP_101348550.1, NZ_CP025268.1, 442101, 442691, -, 591, 26390; cds-WP_000705865.1, NZ_CP025268.1, 442654, 443565, -, 912, 112446; cds-WP_001138904.1, NZ_CP025268.1, 443733, 444224, +, 492, 565; cds-WP_001000963.1, NZ_CP025268.1, 444352, 445716, -, 1365, 2771; cds-WP_000971336.1, NZ_CP025268.1, 445865, 446755, -, 891, 1253013; cds-WP_000019869.1, NZ_CP025268.1, 446767, 447096, -, 330, 457145; cds-WP_000179819.1, NZ_CP025268.1, 447096, 447710, -, 615, 1919279; cds-WP_000467180.1, NZ_CP025268.1, 447700, 449691, -, 1992, 5642384; cds-WP_101348551.1, NZ_CP025268.1, 449713, 450660, -, 948, 2183442; cds-WP_000098429.1, NZ_CP025268.1, 451120, 452595, -, 1476, 316638; cds-WP_001295326.1, NZ_CP025268.1, 452639, 453217, -, 579, 257680; cds-WP_000973448.1, NZ_CP025268.1, 453522, 453839, +, 318, 0; cds-WP_001198386.1, NZ_CP025268.1, 454183, 455481, +, 1299, 1862277; cds-WP_000122253.1, NZ_CP025268.1, 455727, 456350, +, 624, 82332; cds-WP_000130305.1, NZ_CP025268.1, 456476, 457750, +, 1275, 303812; cds-WP_001295325.1, NZ_CP025268.1, 457938, 460292, +, 2355, 601545; cds-WP_001043542.1, NZ_CP025268.1, 460501, 460773, +, 273, 80488; cds-WP_000969372.1, NZ_CP025268.1, 460965, 462836, +, 1872, 1046069; cds-WP_000680312.1, NZ_CP025268.1, 462987, 463358, +, 372, 21257; cds-WP_001194534.1, NZ_CP025268.1, 463452, 463850, +, 399, 0; cds-WP_000817220.1, NZ_CP025268.1, 463902, 464597, -, 696, 647; cds-WP_001238234.1, NZ_CP025268.1, 464662, 466362, -, 1701, 0; cds-WP_001336137.1, NZ_CP025268.1, 466462, 467280, +, 819, 4617; cds-WP_052994949.1, NZ_CP025268.1, 467433, 467891, +, 459, 13994; cds-WP_101348552.1, NZ_CP025268.1, 467921, 469693, +, 1773, 22443; cds-WP_101348553.1, NZ_CP025268.1, 469686, 471467, +, 1782, 155774; cds-WP_000780338.1, NZ_CP025268.1, 471648, 471986, +, 339, 3; cds-WP_000685029.1, NZ_CP025268.1, 472016, 473302, +, 1287, 11102; cds-WP_000075876.1, NZ_CP025268.1, 473351, 474211, -, 861, 1; cds-WP_101348554.1, NZ_CP025268.1, 474429, 475001, +, 573, 13021; cds-WP_000409911.1, NZ_CP025268.1, 475032, 475343, -, 312, 1163; cds-WP_000878140.1, NZ_CP025268.1, 475722, 476075, +, 354, 77593; cds-WP_101348720.1, NZ_CP025268.1, 476117, 477667, -, 1551, 168280; cds-WP_000136192.1, NZ_CP025268.1, 477831, 478301, -, 471, 1585; cds-WP_000102564.1, NZ_CP025268.1, 478417, 478968, -, 552, 62940; cds-WP_001291435.1, NZ_CP025268.1, 479140, 479358, -, 219, 170393; cds-WP_000344800.1, NZ_CP025268.1, 479384, 479758, -, 375, 91333; cds-WP_001132469.1, NZ_CP025268.1, 480304, 483453, -, 3150, 2399433; cds-WP_001295324.1, NZ_CP025268.1, 483476, 484669, -, 1194, 427261; cds-WP_000101737.1, NZ_CP025268.1, 484811, 485458, +, 648, 10946; cds-WP_101348555.1, NZ_CP025268.1, 485586, 488948, +, 3363, 29083; cds-WP_000051153.1, NZ_CP025268.1, 489160, 489321, -, 162, 6; cds-WP_000844874.1, NZ_CP025268.1, 489335, 489862, -, 528, 0; cds-WP_001188905.1, NZ_CP025268.1, 489932, 490309, +, 378, 0; cds-WP_101348556.1, NZ_CP025268.1, 490462, 491013, +, 552, 15010; cds-WP_000122013.1, NZ_CP025268.1, 491142, 493073, +, 1932, 1021894; cds-WP_000467098.1, NZ_CP025268.1, 493126, 493455, +, 330, 212413; cds-WP_001195025.1, NZ_CP025268.1, 493455, 494060, +, 606, 222550; cds-WP_000678201.1, NZ_CP025268.1, 494170, 496044, +, 1875, 319379; cds-WP_001220233.1, NZ_CP025268.1, 496225, 496869, +, 645, 211333; cds-WP_001250103.1, NZ_CP025268.1, 497105, 498067, +, 963, 92; cds-WP_000801813.1, NZ_CP025268.1, 498064, 499023, -, 960, 0; cds-WP_000671574.1, NZ_CP025268.1, 499175, 500479, +, 1305, 221061; cds-WP_000546237.1, NZ_CP025268.1, 500612, 502288, -, 1677, 193789; cds-WP_001251608.1, NZ_CP025268.1, 502526, 503746, -, 1221, 1069; cds-WP_000771748.1, NZ_CP025268.1, 503964, 505616, +, 1653, 79985; cds-WP_000186631.1, NZ_CP025268.1, 505653, 506132, -, 480, 2742; cds-WP_000365173.1, NZ_CP025268.1, 506336, 507130, -, 795, 77021; cds-WP_000806442.1, NZ_CP025268.1, 507268, 507609, +, 342, 17; cds-WP_000083955.1, NZ_CP025268.1, 507925, 510429, -, 2505, 27327; cds-WP_000883034.1, NZ_CP025268.1, 510691, 511623, +, 933, 0; cds-WP_000982172.1, NZ_CP025268.1, 511626, 512918, +, 1293, 52; cds-WP_001026747.1, NZ_CP025268.1, 513043, 513450, +, 408, 1997; cds-WP_000970323.1, NZ_CP025268.1, 513451, 513909, -, 459, 0; cds-WP_000904502.1, NZ_CP025268.1, 513906, 514823, -, 918, 373; cds-WP_001157540.1, NZ_CP025268.1, 514969, 515646, +, 678, 0; cds-WP_001295323.1, NZ_CP025268.1, 515633, 516412, +, 780, 0; cds-WP_001300573.1, NZ_CP025268.1, 516475, 517329, -, 855, 155535; cds-WP_101348557.1, NZ_CP025268.1, 517390, 518199, -, 810, 20690; cds-WP_001295836.1, NZ_CP025268.1, 518189, 518812, -, 624, 0; cds-WP_001110573.1, NZ_CP025268.1, 518783, 519469, +, 687, 0; cds-WP_101348558.1, NZ_CP025268.1, 519466, 521880, +, 2415, 0; cds-WP_000014739.1, NZ_CP025268.1, 522311, 526591, +, 4281, 7556; cds-WP_000877768.1, NZ_CP025268.1, 526631, 526999, +, 369, 0; cds-WP_001300714.1, NZ_CP025268.1, 526999, 527709, +, 711, 0; cds-WP_001320180.1, NZ_CP025268.1, 527690, 527950, +, 261, 0; cds-WP_001297301.1, NZ_CP025268.1, 528007, 528180, +, 174, 0; cds-CXP41_RS02745, NZ_CP025268.1, 528701, 529066, -, 366, 2; cds-WP_001157938.1, NZ_CP025268.1, 529182, 530276, -, 1095, 72936; cds-WP_000460145.1, NZ_CP025268.1, 530345, 531271, -, 927, 0; cds-WP_000776377.1, NZ_CP025268.1, 531501, 531983, +, 483, 0; cds-WP_000141275.1, NZ_CP025268.1, 532061, 532876, +, 816, 34932; cds-WP_001096881.1, NZ_CP025268.1, 532966, 534747, +, 1782, 0; cds-WP_000943544.1, NZ_CP025268.1, 534760, 535536, +, 777, 0; cds-WP_000765839.1, NZ_CP025268.1, 535636, 536514, +, 879, 0; cds-WP_000401100.1, NZ_CP025268.1, 536683, 538137, +, 1455, 246; cds-WP_000006900.1, NZ_CP025268.1, 538197, 539558, +, 1362, 0; cds-WP_001302767.1, NZ_CP025268.1, 539615, 540916, +, 1302, 2423; cds-WP_101348559.1, NZ_CP025268.1, 540938, 542083, +, 1146, 0; cds-WP_000540997.1, NZ_CP025268.1, 542311, 543096, -, 786, 0; cds-WP_101348560.1, NZ_CP025268.1, 543107, 544342, -, 1236, 4; cds-WP_000703900.1, NZ_CP025268.1, 544364, 545413, -, 1050, 0; cds-WP_000580829.1, NZ_CP025268.1, 545730, 547397, +, 1668, 0; cds-WP_000495367.1, NZ_CP025268.1, 547407, 548666, +, 1260, 0; cds-WP_000152519.1, NZ_CP025268.1, 548677, 549492, +, 816, 0; cds-WP_000855379.1, NZ_CP025268.1, 549489, 550382, +, 894, 0; cds-WP_000815571.1, NZ_CP025268.1, 550577, 551644, -, 1068, 103844; cds-WP_001295318.1, NZ_CP025268.1, 551641, 552150, -, 510, 1110; cds-WP_000212247.1, NZ_CP025268.1, 552268, 552990, -, 723, 18664; cds-WP_000255997.1, NZ_CP025268.1, 552993, 553487, -, 495, 164837; cds-WP_000912385.1, NZ_CP025268.1, 553661, 555046, +, 1386, 185111; cds-WP_001143542.1, NZ_CP025268.1, 555082, 555603, -, 522, 31581; cds-WP_000190288.1, NZ_CP025268.1, 555711, 555923, -, 213, 79; cds-WP_000729160.1, NZ_CP025268.1, 555925, 556791, -, 867, 336; cds-WP_000776555.1, NZ_CP025268.1, 557262, 557804, +, 543, 0; cds-WP_000988364.1, NZ_CP025268.1, 558024, 558716, +, 693, 0; cds-WP_001333622.1, NZ_CP025268.1, 558747, 561350, +, 2604, 0; cds-WP_001350487.1, NZ_CP025268.1, 561329, 562369, +, 1041, 0; cds-WP_001255230.1, NZ_CP025268.1, 562380, 562895, +, 516, 0; cds-WP_000805428.1, NZ_CP025268.1, 562898, 563530, -, 633, 0; cds-WP_001350488.1, NZ_CP025268.1, 563865, 565028, -, 1164, 1253; cds-CXP41_RS02925, NZ_CP025268.1, 565148, 565426, -, 279, 0; cds-CXP41_RS02930, NZ_CP025268.1, 565426, 565737, +, 312, 0; cds-CXP41_RS02935, NZ_CP025268.1, 565734, 565823, +, 90, 0; cds-WP_085947771.1-2, NZ_CP025268.1;NZ_CP025268.1, 565892;566153, 566153;567054, +;+, 1163, 6; cds-CXP41_RS02945, NZ_CP025268.1, 567085, 567297, +, 213, 0; cds-WP_001070439.1, NZ_CP025268.1, 567365, 567697, +, 333, 545; cds-WP_001372443.1, NZ_CP025268.1, 567745, 567894, +, 150, 0; cds-WP_000709082.1, NZ_CP025268.1, 567952, 569478, +, 1527, 3; cds-WP_001306955.1, NZ_CP025268.1, 569943, 570494, +, 552, 0; cds-WP_000881075.1, NZ_CP025268.1, 570504, 571301, +, 798, 724; cds-WP_001303586.1, NZ_CP025268.1, 571418, 571519, +, 102, 0; cds-WP_001054340.1, NZ_CP025268.1, 571516, 571971, +, 456, 0; cds-WP_000224915.1, NZ_CP025268.1, 571971, 572141, +, 171, 0; cds-WP_000774486.1, NZ_CP025268.1, 572134, 572424, +, 291, 0; cds-WP_001099712.1, NZ_CP025268.1, 572421, 572783, +, 363, 0; cds-WP_000971055.1, NZ_CP025268.1, 572780, 572920, +, 141, 0; cds-WP_001204791.1, NZ_CP025268.1, 573006, 573389, +, 384, 0; cds-WP_000019403.1-2, NZ_CP025268.1, 573787, 574767, -, 981, 227007; cds-WP_000737282.1, NZ_CP025268.1, 574808, 575875, -, 1068, 221952; cds-WP_000839596.1, NZ_CP025268.1, 576448, 576663, +, 216, 202; cds-WP_001135281.1, NZ_CP025268.1, 576663, 577160, +, 498, 0; cds-WP_001228697.1, NZ_CP025268.1, 577377, 577559, +, 183, 3474; cds-WP_000738500.1, NZ_CP025268.1, 577650, 577943, -, 294, 3921; cds-WP_000079503.1, NZ_CP025268.1, 578234, 578644, -, 411, 0; cds-WP_001031427.1, NZ_CP025268.1, 578930, 579136, +, 207, 7916; cds-WP_001421937.1, NZ_CP025268.1, 579301, 579495, -, 195, 0; cds-WP_000453566.1, NZ_CP025268.1, 579884, 580429, +, 546, 0; cds-CXP41_RS24350, NZ_CP025268.1, 580404, 580712, +, 309, 0; cds-CXP41_RS24355, NZ_CP025268.1, 580710, 581147, +, 438, 0; cds-WP_026089880.1, NZ_CP025268.1, 581201, 581827, -, 627, 0; cds-CXP41_RS03090, NZ_CP025268.1, 581924, 582184, +, 261, 0; cds-WP_000386784.1, NZ_CP025268.1, 582730, 583479, +, 750, 0; cds-WP_001201845.1, NZ_CP025268.1, 583729, 584682, -, 954, 997472; cds-WP_101348561.1, NZ_CP025268.1, 585196, 585957, -, 762, 0; cds-WP_001224604.1, NZ_CP025268.1, 586140, 587030, -, 891, 12; cds-WP_000662357.1, NZ_CP025268.1, 587031, 590003, -, 2973, 143238; cds-WP_000383932.1, NZ_CP025268.1, 589990, 592227, -, 2238, 93; cds-WP_000253839.1, NZ_CP025268.1, 592377, 593819, -, 1443, 27915; cds-WP_000770953.1, NZ_CP025268.1, 593809, 594492, -, 684, 45025; cds-WP_000074234.1, NZ_CP025268.1, 594649, 596022, +, 1374, 0; cds-WP_000709870.1, NZ_CP025268.1, 596180, 596512, +, 333, 0; cds-WP_000717157.1, NZ_CP025268.1, 596528, 597751, +, 1224, 0; cds-WP_000573945.1, NZ_CP025268.1, 597763, 600906, +, 3144, 53049; cds-WP_000786319.1, NZ_CP025268.1, 601008, 602384, +, 1377, 1691; cds-WP_001153148.1, NZ_CP025268.1, 602465, 603712, -, 1248, 117698; cds-WP_000351487.1, NZ_CP025268.1, 603820, 604473, -, 654, 474; cds-WP_000360951.1, NZ_CP025268.1, 604567, 604935, -, 369, 275; cds-WP_000682509.1, NZ_CP025268.1, 605000, 605248, -, 249, 22; cds-WP_001130654.1, NZ_CP025268.1, 605314, 606432, -, 1119, 100888; cds-WP_000956455.1, NZ_CP025268.1, 606885, 607037, +, 153, 0; cds-WP_001300563.1-2, NZ_CP025268.1, 607114, 608226, +, 1113, 95065; cds-WP_001749708.1, NZ_CP025268.1, 608508, 609128, -, 621, 0; cds-WP_001034943.1, NZ_CP025268.1, 609303, 611543, -, 2241, 55; cds-WP_000125843.1, NZ_CP025268.1, 611786, 612988, +, 1203, 4; cds-WP_000885798.1, NZ_CP025268.1, 612991, 613209, +, 219, 0; cds-WP_000077805.1, NZ_CP025268.1, 613206, 617087, +, 3882, 58; cds-WP_000096692.1, NZ_CP025268.1, 617303, 618436, +, 1134, 0; cds-WP_000140647.1, NZ_CP025268.1, 618433, 619248, -, 816, 997; cds-WP_000640988.1, NZ_CP025268.1, 619245, 620237, -, 993, 79105; cds-WP_000016947.1, NZ_CP025268.1, 620234, 621238, -, 1005, 155930; cds-WP_001041786.1, NZ_CP025268.1, 621349, 622599, +, 1251, 406782; cds-WP_001234311.1, NZ_CP025268.1, 622603, 623559, -, 957, 3754; cds-WP_000381303.1, NZ_CP025268.1, 623934, 625109, +, 1176, 1; cds-WP_000026812.1, NZ_CP025268.1, 625119, 626729, +, 1611, 0; cds-WP_001007138.1, NZ_CP025268.1, 626743, 627600, +, 858, 19; cds-WP_000347651.1, NZ_CP025268.1, 627600, 628346, +, 747, 0; cds-WP_000637953.1, NZ_CP025268.1, 628349, 628762, +, 414, 0; cds-WP_101348562.1, NZ_CP025268.1, 628943, 631048, +, 2106, 0; cds-WP_000460431.1, NZ_CP025268.1, 631231, 631428, +, 198, 0; cds-WP_001120441.1, NZ_CP025268.1, 631438, 632526, -, 1089, 0; cds-WP_000183907.1, NZ_CP025268.1, 632635, 633795, +, 1161, 9; cds-WP_000502945.1, NZ_CP025268.1, 633796, 634425, -, 630, 9; cds-WP_000029814.1, NZ_CP025268.1, 634398, 635618, -, 1221, 0; cds-
WP_000002474.1, NZ_CP025268.1, 635765, 636667, -, 903, 0; cds-WP_000913829.1, NZ_CP025268.1, 636876, 637622, -, 747, 0; cds-WP_000052796.1, NZ_CP025268.1, 637994, 638557, +, 564, 264945; cds-WP_000887629.1, NZ_CP025268.1, 638802, 640367, +, 1566, 69361; cds-WP_000278509.1, NZ_CP025268.1, 640488, 640916, -, 429, 0; cds-WP_000646173.1, NZ_CP025268.1, 641137, 642375, +, 1239, 0; cds-WP_000089731.1, NZ_CP025268.1, 642606, 643016, -, 411, 42; cds-WP_000643397.1, NZ_CP025268.1, 643246, 644052, -, 807, 589; cds-WP_000050323.1, NZ_CP025268.1, 644166, 645629, -, 1464, 0; cds-WP_000062457.1, NZ_CP025268.1, 645680, 646558, -, 879, 33515; cds-WP_000550422.1, NZ_CP025268.1, 646533, 647084, -, 552, 0; cds-WP_101348563.1, NZ_CP025268.1, 647088, 648620, -, 1533, 0; cds-WP_000622357.1, NZ_CP025268.1, 648631, 649539, -, 909, 0; cds-WP_000700703.1, NZ_CP025268.1, 649536, 649832, -, 297, 0; cds-WP_000467705.1, NZ_CP025268.1, 649847, 650905, -, 1059, 0; cds-WP_000939767.1, NZ_CP025268.1, 651284, 652942, +, 1659, 0; cds-WP_000126500.1, NZ_CP025268.1, 652911, 653591, +, 681, 4924; cds-WP_000955063.1, NZ_CP025268.1, 653632, 655017, -, 1386, 0; cds-WP_001103094.1, NZ_CP025268.1, 655606, 656166, +, 561, 3; cds-WP_000034825.1, NZ_CP025268.1, 656341, 656550, +, 210, 3339; cds-WP_000939747.1, NZ_CP025268.1, 656604, 656987, -, 384, 9664; cds-CXP41_RS03465, NZ_CP025268.1, 657080, 657867, +, 788, 0; cds-WP_000503931.1, NZ_CP025268.1, 657996, 658199, +, 204, 3; cds-WP_000042632.1, NZ_CP025268.1, 658300, 659265, -, 966, 118663; cds-WP_000378026.1, NZ_CP025268.1, 659474, 660427, -, 954, 2434; cds-WP_101348564.1, NZ_CP025268.1, 660686, 661327, -, 642, 1165; cds-WP_000850550.1, NZ_CP025268.1, 661428, 661691, -, 264, 94; cds-WP_001092082.1, NZ_CP025268.1, 661801, 663012, -, 1212, 410974; cds-WP_001231430.1, NZ_CP025268.1, 663151, 664239, -, 1089, 336757; cds-WP_000131719.1, NZ_CP025268.1, 664250, 665362, -, 1113, 296774; cds-WP_000776191.1, NZ_CP025268.1, 665365, 667266, -, 1902, 1836341; cds-WP_000776104.1, NZ_CP025268.1, 667297, 667764, -, 468, 282734; cds-WP_001161664.1, NZ_CP025268.1, 667768, 668085, -, 318, 170801; cds-WP_001241872.1, NZ_CP025268.1, 668345, 668956, -, 612, 94; cds-WP_000838889.1, NZ_CP025268.1, 668980, 669621, -, 642, 186507; cds-WP_000620535.1, NZ_CP025268.1, 669623, 670654, -, 1032, 211024; cds-WP_001269673.1, NZ_CP025268.1, 670654, 671235, -, 582, 173175; cds-WP_101348565.1, NZ_CP025268.1, 671250, 673832, -, 2583, 2149953; cds-WP_001044880.1, NZ_CP025268.1, 674067, 674549, +, 483, 0; cds-CXP41_RS03565, NZ_CP025268.1, 674619, 675595, -, 977, 0; cds-WP_000367023.1, NZ_CP025268.1, 675759, 676466, +, 708, 0; cds-WP_101348566.1, NZ_CP025268.1, 676463, 677890, +, 1428, 0; cds-WP_001035674.1, NZ_CP025268.1, 677900, 678454, -, 555, 0; cds-WP_101348567.1, NZ_CP025268.1, 678556, 679263, +, 708, 0; cds-WP 001300403.1, NZ_CP025268.1, 679260, 680711, +, 1452, 2; cds-WP 000367875.1, NZ_CP025268.1, 680771, 682441, -, 1671, 0; cds-WP_001207520.1, NZ_CP025268.1, 682525, 683460, -, 936, 1897; cds-WP_000631384.1, NZ_CP025268.1, 683578, 684303, -, 726, 4; cds-WP_000272824.1, NZ_CP025268.1, 684303, 684977, -, 675, 7697; cds-WP_000020941.1, NZ_CP025268.1, 684977, 685717, -, 741, 1140; cds-WP_001177086.1, NZ_CP025268.1, 685887, 686795, -, 909, 412365; cds-WP_000019403.1-3, NZ_CP025268.1, 687045, 688025, -, 981, 165883; cds-WP_000853021.1, NZ_CP025268.1, 688391, 689929, -, 1539, 270915; cds-WP_001278605.1, NZ_CP025268.1, 689954, 690832, -, 879, 606116; cds-WP_000084469.1, NZ_CP025268.1, 690922, 691389, -, 468, 110931; cds-WP_001018040.1, NZ_CP025268.1, 691386, 692426, -, 1041, 271361; cds-WP_000162740.1, NZ_CP025268.1, 692579, 694003, -, 1425, 468465; cds-WP_000184541.1, NZ_CP025268.1, 694149, 695324, +, 1176, 30490; cds-WP_000337077.1, NZ_CP025268.1, 696561, 698225, -, 1665, 2104; cds-WP_000153129.1, NZ_CP025268.1, 698622, 699374, -, 753, 45908; cds-WP_000187594.1, NZ_CP025268.1, 699422, 700642, -, 1221, 134199; cds-WP_000271153.1, NZ_CP025268.1, 700651, 701799, -, 1149, 13807; cds-WP_001237072.1, NZ_CP025268.1, 701859, 702659, -, 801, 0; cds-WP_001023093.1, NZ_CP025268.1, 702992, 704938, +, 1947, 155467; cds-WP_001287154.1, NZ_CP025268.1, 705141, 706805, +, 1665, 945796; cds-WP_001258819.1, NZ_CP025268.1, 707382, 708788, +, 1407, 11508; cds-WP_000733579.1, NZ_CP025268.1, 708838, 709164, +, 327, 1694; cds-WP_000131702.1, NZ_CP025268.1, 709248, 709694, -, 447, 125843; cds-WP_001406816.1, NZ_CP025268.1, 709687, 709773, -, 87, 52385; cds-WP_001018618.1, NZ_CP025268.1, 709983, 710513, -, 531, 592988; cds-WP_101348568.1, NZ_CP025268.1, 710653, 710946, -, 294, 26661; cds-WP_000773288.1, NZ_CP025268.1, 711086, 711850, -, 765, 790; cds-WP_000848387.1, NZ_CP025268.1, 712035, 712580, +, 546, 108189; cds-WP_001297249.1, NZ_CP025268.1, 712606, 714246, +, 1641, 208785; cds-WP_000856353.1, NZ_CP025268.1, 714460, 714954, +, 495, 35278; cds-CXP41 R503795, NZ_CP025268.1, 714995, 715645, -, 651, 0; cds-CXP41_RS03800, NZ_CP025268.1, 715769, 715918, -, 150, 0; cds-WP_000075845.1, NZ_CP025268.1, 715994, 717313, -, 1320, 0; cds-WP_000040195.1, NZ_CP025268.1, 717310, 719508, -, 2199, 3; cds-WP_211180488.1, NZ_CP025268.1, 719766, 719870, -, 105, 0; cds-WP_000186076.1, NZ_CP025268.1, 720104, 720781, -, 678, 0; cds-CXP41_RS03825, NZ_CP025268.1, 720778, 723463, -, 2686, 9; cds-WP_001300431.1, NZ_CP025268.1, 723456, 724028, -, 573, 0; cds-WP_000087939.1, NZ_CP025268.1, 724037, 726085, -, 2049, 549; cds-WP_000741129.1, NZ_CP025268.1, 726108, 727781, -, 1674, 93; cds-WP_001272653.1, NZ_CP025268.1, 727781, 727870, -, 90, 0; cds-WP_000424924.1, NZ_CP025268.1, 728183, 728389, +, 207, 0; cds-WP_000015200.1, NZ_CP025268.1, 728632, 732825, +, 4194, 14025; cds-WP_000873944.1, NZ_CP025268.1, 732825, 733151, +, 327, 0; cds-CXP41_RS03865, NZ_CP025268.1, 733197, 734702, +, 1506, 0; cds-WP_000832338.1, NZ_CP025268.1, 734699, 735268, +, 570, 0; cds-CXP41_RS03875, NZ_CP025268.1, 735494, 735727, +, 234, 0; cds-CXP41_RS03880, NZ_CP025268.1, 735874, 737010, +, 1137, 80; cds-CXP41_RS03885, NZ_CP025268.1, 737141, 737899, +, 759, 0; cds-WP_001053305.1, NZ_CP025268.1, 738050, 738559, +, 510, 0; cds-WP_000207142.1, NZ_CP025268.1, 738556, 739974, +, 1419, 1; cds-WP_001032689.1, NZ_CP025268.1, 740124, 741605, -, 1482, 95222; cds-WP_000798871.1, NZ_CP025268.1, 741876, 742619, +, 744, 24496; cds-WP_001188343.1, NZ_CP025268.1, 742642, 743298, +, 657, 240; cds-WP_000912724.1, NZ_CP025268.1, 743292, 744224, +, 933, 5715; cds-WP_000687112.1, NZ_CP025268.1, 744214, 744948, +, 735, 0; cds-WP_001113989.1, NZ_CP025268.1, 744984, 745775, +, 792, 10705; cds-WP_001336221.1, NZ_CP025268.1, 745772, 746818, -, 1047, 0; cds-WP_001272410.1, NZ_CP025268.1, 746970, 748031, -, 1062, 0; cds-WP_000142799.1, NZ_CP025268.1, 748028, 748756, -, 729, 0; cds-WP_001310647.1, NZ_CP025268.1, 748771, 751227, -, 2457, 0; cds-WP_000472412.1, NZ_CP025268.1, 751278, 751844, -, 567, 0; cds-WP_000785834.1, NZ_CP025268.1, 752234, 753517, -, 1284, 98508; cds-WP_001305582.1, NZ_CP025268.1, 753723, 753830, -, 108, 688; cds-WP_000263347.1, NZ_CP025268.1, 754211, 754615, +, 405, 47351; cds-WP_000254365.1, NZ_CP025268.1, 754609, 754956, +, 348, 17; cds-WP_000775540.1, NZ_CP025268.1, 754956, 756722, +, 1767, 35784; cds-WP_001235254.1, NZ_CP025268.1, 756738, 757454, +, 717, 139338; cds-WP_001181473.1, NZ_CP025268.1, 757755, 760556, +, 2802, 762321; cds-WP_000099823.1, NZ_CP025268.1, 760571, 761788, +, 1218, 228455; cds-WP_001048602.1, NZ_CP025268.1, 762063, 763229, +, 1167, 671; cds-WP_000025458.1, NZ_CP025268.1, 763229, 764098, +, 870, 14684; cds-WP_000509902.1, NZ_CP025268.1, 764202, 764924, -, 723, 0; cds-WP_001054864.1, NZ_CP025268.1, 765093, 767009, +, 1917, 35; cds-WP_000648976.1, NZ_CP025268.1, 767027, 769660, +, 2634, 8; cds-WP_000884361.1, NZ_CP025268.1, 770507, 772075, +, 1569, 1193815; cds-WP_000568275.1, NZ_CP025268.1, 772091, 773230, +, 1140, 1803969; cds-WP_000270282.1, NZ_CP025268.1, 773245, 773358, +, 114, 34634; cds-WP_000034602.1, NZ_CP025268.1, 773358, 773651, +, 294, 114145; cds-WP_001098384.1, NZ_CP025268.1, 773801, 774205, +, 405, 37149; cds-WP_000131314.1, NZ_CP025268.1, 774202, 774894, +, 693, 142109; cds-WP_000090097.1, NZ_CP025268.1, 774898, 775326, +, 429, 106443; cds-WP_000030637.1, NZ_CP025268.1, 775391, 776656, +, 1266, 255216; cds-WP_001295307.1, NZ_CP025268.1, 776789, 778081, +, 1293, 186560; cds-WP_001295306.1, NZ_CP025268.1, 778116, 778637, +, 522, 39438; cds-WP_000097571.1, NZ_CP025268.1, 778647, 779438, +, 792, 327012; cds-WP_000115290.1, NZ_CP025268.1, 781134, 782177, +, 1044, 0; cds-WP_000345410.1, NZ_CP025268.1, 782215, 782934, +, 720, 51009; cds-WP_000951292.1, NZ_CP025268.1, 782931, 783872, -, 942, 1; cds-WP_000784351.1, NZ_CP025268.1, 783986, 784366, -, 381, 27; cds-WP_001109196.1, NZ_CP025268.1, 784682, 785734, +, 1053, 13; cds-WP_001295305.1, NZ_CP025268.1, 785892, 786644, -, 753, 335355; cds-WP_101348569.1, NZ_CP025268.1, 786846, 787895, -, 1050, 19212; cds-WP_000191497.1, NZ_CP025268.1, 787883, 788929, -, 1047, 132; cds-WP_001265438.1, NZ_CP025268.1, 788939, 789955, -, 1017, 38897; cds-WP_000096869.1, NZ_CP025268.1, 790216, 791688, -, 1473, 45779; cds-WP_001147439.1, NZ_CP025268.1, 791756, 792544, -, 789, 1106; cds-WP_000891515.1, NZ_CP025268.1, 792673, 792822, +, 150, 0; cds-WP_000101984.1, NZ_CP025268.1, 792989, 793762, +, 774, 0; cds-WP_000604034.1, NZ_CP025268.1, 793762, 794451, +, 690, 10876; cds-WP_000891692.1, NZ_CP025268.1, 794454, 795512, +, 1059, 1316; cds-WP_001300666.1, NZ_CP025268.1, 795513, 796331, -, 819, 80942; cds-WP_000815435.1, NZ_CP025268.1, 796486, 797481, +, 996, 73; cds-WP_001350493.1, NZ_CP025268.1, 797522, 798475, -, 954, 0; cds-WP_000723652.1, NZ_CP025268.1, 798659, 799711, +, 1053, 0; cds-WP_001036475.1, NZ_CP025268.1, 799787, 801220, +, 1434, 0; cds-WP_000593938.1, NZ_CP025268.1, 801403, 803664, +, 2262, 0; cds-WP_001091569.1, NZ_CP025268.1, 803898, 805181, -, 1284, 88979; cds-WP_000533640.1, NZ_CP025268.1, 805316, 806386, -, 1071, 0; cds-WP_002414258.1, NZ_CP025268.1, 806364, 806582, -, 219, 0; cds-WP_000545733.1, NZ_CP025268.1, 806622, 806789, -, 168, 0; cds-WP_000026224.1, NZ_CP025268.1, 806878, 807159, -, 282, 0; cds-WP_001289873.1, NZ_CP025268.1, 807351, 807899, -, 549, 0; cds-WP_000763367.1, NZ_CP025268.1, 807896, 808117, -, 222, 0; cds-CXP41_RS04275, NZ_CP025268.1, 808216, 808432, -, 217, 0; cds-WP_000548551.1, NZ_CP025268.1, 808509, 808700, -, 192, 0; cds-WP_000149542.1, NZ_CP025268.1, 808673, 808855, -, 183, 0; cds-WP_000186891.1, NZ_CP025268.1, 808852, 809532, -, 681, 0; cds-WP_000100844.1, NZ_CP025268.1, 809529, 810314, -, 786, 0; cds-WP_000995451.1, NZ_CP025268.1, 810320, 810616, -, 297, 0; cds-WP_000372937.1, NZ_CP025268.1, 810691, 810834, -, 144, 0; cds-WP_157825722.1, NZ_CP025268.1, 810803, 810967, -, 165, 0; cds-WP_000065374.1, NZ_CP025268.1, 811040, 811408, -, 369, 0; cds-WP_000213975.1, NZ_CP025268.1, 811591, 811791, -, 201, 0; cds-WP_000281856.1, NZ_CP025268.1, 812058, 812540, +, 483, 0; cds-WP_001564525.1, NZ_CP025268.1, 812541, 812864, -, 324, 0; cds-WP_001245922.1, NZ_CP025268.1, 813329, 813763, -, 435, 0; cds-WP_000788349.1, NZ_CP025268.1, 813779, 814618, -, 840, 0; cds-WP_101348571.1, NZ_CP025268.1, 814731, 815444, -, 714, 0; cds-WP_000088605.1, NZ_CP025268.1, 815706, 816329, +, 624, 0; cds-WP_000027050.1, NZ_CP025268.1, 816414, 817274, +, 861, 0; cds-WP_000118826.1, NZ_CP025268.1, 817488, 818642, +, 1155, 0; cds-WP_000246761.1, NZ_CP025268.1, 818629, 819384, +, 756, 0; cds-WP_000044843.1, NZ_CP025268.1, 819377, 820054, +, 678, 0; cds-WP_000042533.1, NZ_CP025268.1, 820633, 822654, +, 2022, 328; cds-WP_001295302.1, NZ_CP025268.1, 822846, 823754, -, 909, 10; cds-WP_001350494.1, NZ_CP025268.1, 824151, 825140, +, 990, 103496; cds-WP_000084639.1, NZ_CP025268.1, 825162, 825674, +, 513, 6608; cds-WP_000080885.1, NZ_CP025268.1, 825677, 826162, +, 486, 9625; cds-WP_000598619.1, NZ_CP025268.1, 826155, 826400, +, 246, 3094; cds-WP_000852296.1, NZ_CP025268.1, 826402, 826854, +, 453, 44576; cds-WP_000373624.1, NZ_CP025268.1, 826991, 827695, +, 705, 583; cds-WP_000446911.1, NZ_CP025268.1, 827900, 828613, +, 714, 68; cds-WP_000045450.1, NZ_CP025268.1, 828649, 829605, -, 957, 0; cds-WP _000650337.1, NZ_CP025268.1, 829605, 830846, -, 1242, 0; cds-WP_001113348.1, NZ_CP025268.1, 830843, 831604, -, 762, 0; cds-WP_000871982.1, NZ_CP025268.1, 831737, 832147, +, 411, 38; cds-WP_101348572.1, NZ_CP025268.1, 832109, 833215, -, 1107, 290; cds-WP_000070131.1, NZ_CP025268.1,
833226, 834359, -, 1134, 14; cds-WP_000996107.1, NZ_CP025268.1, 834352, 836088, -, 1737, 24026; cds-WP_000976409.1, NZ_CP025268.1, 836081, 837079, -, 999, 8; cds-WP_001296991.1, NZ_CP025268.1, 837079, 837750, -, 672, 0; cds-WP_000007101.1, NZ_CP025268.1, 837979, 839343, +, 1365, 459241; cds-WP_001145126.1, NZ_CP025268.1, 839575, 840057, -, 483, 24; cds-WP_001340191.1, NZ_CP025268.1, 840177, 842327, +, 2151, 550154; cds-WP_000386551.1, NZ_CP025268.1, 842355, 843317, +, 963, 0; cds-WP_000443530.1, NZ_CP025268.1, 843458, 844543, +, 1086, 1; cds-WP_211180490.1, NZ_CP025268.1, 844610, 844669, +, 60, 6; cds-WP_000849301.1, NZ_CP025268.1, 844772, 845032, -, 261, 215; cds-WP_000146343.1, NZ_CP025268.1, 845297, 845563, -, 267, 19; cds-WP_000990177.1, NZ_CP025268.1, 845637, 846314, -, 678, 112175; cds-WP_000430057.1, NZ_CP025268.1, 846356, 848638, -, 2283, 13; cds-WP_000710619.1, NZ_CP025268.1, 848903, 849163, -, 261, 0; cds-WP_001295299.1, NZ_CP025268.1, 849439, 850365, +, 927, 48; cds-WP_001267253.1, NZ_CP025268.1, 850362, 852587, -, 2226, 68; cds-WP_000569083.1, NZ_CP025268.1, 852848, 853570, -, 723, 121985; cds-WP_001159065.1, NZ_CP025268.1, 853567, 854226, -, 660, 20435; cds-WP_000843866.1, NZ_CP025268.1, 854365, 855111, -, 747, 73058; cds-WP_000100800.1, NZ_CP025268.1, 855515, 856018, -, 504, 0; cds-WP_001295297.1, NZ_CP025268.1, 856317, 857204, -, 888, 115; cds-WP_148936914.1, NZ_CP025268.1, 857439, 857504, +, 66, 10483; cds-WP_001295296.1, NZ_CP025268.1, 857557, 858072, +, 516, 321947; cds-WP_001054678.1, NZ_CP025268.1, 858121, 859704, -, 1584, 36526; cds-WP_001001761.1, NZ_CP025268.1, 859976, 860104, -, 129, 13035; cds-WP_000091016.1, NZ_CP025268.1, 860290, 860757, +, 468, 3969; cds-WP_000056457.1, NZ_CP025268.1, 860754, 861872, +, 1119, 54467; cds-WP_001056384.1, NZ_CP025268.1, 861931, 862851, -, 921, 5940; cds-WP_000961458.1, NZ_CP025268.1, 863070, 864662, +, 1593, 208011; cds-WP_000168797.1, NZ_CP025268.1, 864903, 866168, -, 1266, 73; cds-WP_000114272.1, NZ_CP025268.1, 866320, 867135, -, 816, 7921; cds-WP_101348573.1, NZ_CP025268.1, 867281, 869713, -, 2433, 0; cds-WP_001295295.1, NZ_CP025268.1, 869719, 870618, -, 900, 0; cds-WP_001336208.1, NZ_CP025268.1, 870749, 871411, +, 663, 56; cds-WP_000829217.1, NZ_CP025268.1, 871487, 872236, -, 750, 52; cds-WP_000397340.1, NZ_CP025268.1, 872236, 873471, -, 1236, 51680; cds-WP_000513781.1, NZ_CP025268.1, 873675, 874640, +, 966, 11; cds-WP_001301279.1, NZ_CP025268.1, 874627, 876498, +, 1872, 195083; cds-WP_000090140.1, NZ_CP025268.1, 876518, 878056, +, 1539, 43681; cds-WP_000936043.1, NZ_CP025268.1, 878074, 878994, +, 921, 43559; cds-WP_001236051.1, NZ_CP025268.1, 878997, 879908, +, 912, 4; cds-WP_001360263.1, NZ_CP025268.1, 880086, 882434, +, 2349, 112; cds-WP_000086904.1, NZ_CP025268.1, 882442, 883770, +, 1329, 19002; cds-WP_000049367.1, NZ_CP025268.1, 883817, 885142, -, 1326, 50494; cds-WP_000497137.1, NZ_CP025268.1, 885355, 885738, +, 384, 0; cds-WP 000555031.1, NZ_CP025268.1, 885849, 886964, +, 1116, 30; cds-WP_001295292.1, NZ_CP025268.1, 886961, 887587, -, 627, 345; cds-WP_001300708.1, NZ_CP025268.1, 887834, 889036, +, 1203, 36131; cds-WP_000450121.1, NZ_CP025268.1, 889083, 889841, -, 759, 27098; cds-WP_001295291.1, NZ_CP025268.1, 889899, 890495, -, 597, 4396; cds-WP_001180089.1, NZ_CP025268.1, 890780, 892012, +, 1233, 34510; cds-WP_000605480.1, NZ_CP025268.1, 892053, 892337, -, 285, 37095; cds-WP_000023565.1, NZ_CP025268.1, 892423, 893238, -, 816, 74458; cds-WP_000217859.1, NZ_CP025268.1, 893238, 894446, -, 1209, 8253; cds-WP_001295289.1, NZ_CP025268.1, 894530, 895066, +, 537, 0; cds-WP_001024876.1, NZ_CP025268.1, 895241, 896926, -, 1686, 128295; cds-WP_000681108.1, NZ_CP025268.1, 897196, 897573, +, 378, 971; cds-WP_001195240.1, NZ_CP025268.1, 897603, 897860, -, 258, 0; cds-WP 001201560.1, NZ_CP025268.1, 898020, 898307, +, 288, 721; cds-WP_000189159.1, NZ_CP025268.1, 898291, 899013, +, 723, 115708; cds-WP_000684321.1, NZ_CP025268.1, 899074, 899976, +, 903, 173825; cds-WP_000203025.1, NZ_CP025268.1, 900064, 900540, +, 477, 80294; cds-WP_000126072.1, NZ_CP025268.1, 900891, 902003, +, 1113, 43415; cds-WP_000996005.1, NZ_CP025268.1, 902098, 903231, +, 1134, 24197; cds-WP_000105430.1, NZ_CP025268.1, 903241, 904194, +, 954, 0; cds-WP_001061658.1, NZ_CP025268.1, 904191, 905036, +, 846, 0; cds-WP_000389260.1, NZ_CP025268.1, 905096, 905584, +, 489, 5; cds-WP_001149682.1, NZ_CP025268.1, 905625, 906752, +, 1128, 195; cds-WP_001295339.1, NZ_CP025268.1, 906951, 907682, -, 732, 63741; cds-WP_000464491.1, NZ_CP025268.1, 907973, 908641, -, 669, 30919; cds-WP_001001691.1, NZ_CP025268.1, 908641, 909357, -, 717, 101531; cds-WP_000756569.1, NZ_CP025268.1, 909364, 910095, -, 732, 13200; cds-WP_000027205.1, NZ_CP025268.1, 910113, 910841, -, 729, 37369; cds-WP_001270734.1, NZ_CP025268.1, 911059, 911574, -, 516, 178; cds-WP_001160737.1, NZ_CP025268.1, 911700, 912023, +, 324, 12; cds-WP_001252135.1, NZ_CP025268.1, 912020, 912850, +, 831, 11; cds-WP_001338420.1, NZ_CP025268.1, 912847, 913860, -, 1014, 667; cds-WP_001136554.1, NZ_CP025268.1, 913959, 915389, -, 1431, 12119; cds-WP_000566376.1, NZ_CP025268.1, 915400, 916401, -, 1002, 869; cds-WP_000815337.1, NZ_CP025268.1, 916438, 918156, -, 1719, 2; cds-WP_000178677.1, NZ_CP025268.1, 918289, 919257, -, 969, 0; cds-WP_000458809.1, NZ_CP025268.1, 919269, 920921, -, 1653, 0; cds-WP_000491142.1, NZ_CP025268.1, 921065, 921964, -, 900, 100; cds-WP_001298299.1, NZ_CP025268.1, 922459, 923154, -, 696, 35; cds-WP_000599802.1, NZ_CP025268.1, 923580, 925238, +, 1659, 555482; cds-WP_001380339.1, NZ_CP025268.1, 925235, 926191, -, 957, 146922; cds-WP_000746442.1, NZ_CP025268.1, 926342, 927457, +, 1116, 4; cds-WP_000188144.1, NZ_CP025268.1, 927454, 929400, +, 1947, 77152; cds-WP_000410785.1, NZ_CP025268.1, 929473, 929697, -, 225, 6; cds-WP_001406719.1, NZ_CP025268.1, 929847, 929921, +, 75, 0; cds-WP_000520781.1, NZ_CP025268.1, 930020, 930340, +, 321, 0; cds-WP_000934041.1, NZ_CP025268.1, 930371, 932647, +, 2277, 422920; cds-WP_001040187.1, NZ_CP025268.1, 933332, 933550, -, 219, 160435; cds-WP_001241678.1, NZ_CP025268.1, 933835, 934539, -, 705, 32; cds-WP_001202177.1, NZ_CP025268.1, 934581, 936302, -, 1722, 56628; cds-WP_001043598.1, NZ_CP025268.1, 936303, 938069, -, 1767, 46988; cds-WP_000537418.1, NZ_CP025268.1, 938192, 939157, -, 966, 134577; cds-WP_000228473.1, NZ_CP025268.1, 939701, 940195, +, 495, 45903; cds-WP_000076967.1, NZ_CP025268.1, 940330, 944319, +, 3990, 249622; cds-WP_001295343.1, NZ_CP025268.1, 944478, 945089, +, 612, 196847; cds-WP_000067755.1, NZ_CP025268.1, 945100, 946443, +, 1344, 183583; cds-WP_000886683.1, NZ_CP025268.1, 946534, 947826, +, 1293, 250058; cds-WP_000850303.1, NZ_CP025268.1, 948065, 950509, +, 2445, 2162; cds-WP_000213098.1, NZ_CP025268.1, 950520, 951137, +, 618, 1; cds-WP_000534637.1, NZ_CP025268.1, 951139, 952002, +, 864, 0; cds-WP_000165879.1, NZ_CP025268.1, 952037, 952663, -, 627, 0; cds-WP_000109259.1, NZ_CP025268.1, 952977, 954125, +, 1149, 0; cds-WP_000918506.1, NZ_CP025268.1, 954335, 955765, +, 1431, 6; cds-WP_001242684.1, NZ_CP025268.1, 955766, 956674, -, 909, 0; cds-WP_001190363.1, NZ_CP025268.1, 956774, 957364, +, 591, 0; cds-WP_000111043.1, NZ_CP025268.1, 957446, 958186, -, 741, 1374; cds-WP_001292822.1, NZ_CP025268.1, 958378, 960660, -, 2283, 1333550; cds-WP_000642546.1, NZ_CP025268.1, 960715, 961572, -, 858, 25; cds-WP_001295344.1, NZ_CP025268.1, 961978, 963738, -, 1761, 155681; cds-WP_000642849.1, NZ_CP025268.1, 963868, 964560, +, 693, 0; cds-WP_000057138.1, NZ_CP025268.1, 964759, 965847, +, 1089, 203547; cds-WP_000445231.1, NZ_CP025268.1, 965918, 967201, +, 1284, 198564; cds-WP_001350496.1, NZ_CP025268.1, 967370, 968134, +, 765, 0; cds-WP_000125016.1, NZ_CP025268.1, 968307, 968990, +, 684, 121094; cds-WP_000140327.1, NZ_CP025268.1, 969101, 970774, +, 1674, 19851014; cds-WP_000167336.1, NZ_CP025268.1, 970934, 971218, +, 285, 87540; cds-WP_000705764.1, NZ_CP025268.1, 971426, 973690, +, 2265, 92592; cds-WP_000551270.1, NZ_CP025268.1, 973727, 975475, +, 1749, 153829; cds-WP_000570539.1, NZ_CP025268.1, 975472, 976458, +, 987, 27446; cds-WP_000056538.1, NZ_CP025268.1, 976495, 977727, +, 1233, 29682; cds-WP_000350058.1, NZ_CP025268.1, 977779, 977961, +, 183, 13909; cds-WP_000011603.1, NZ_CP025268.1, 977958, 978704, +, 747, 204944; cds-WP_000436922.1, NZ_CP025268.1, 978858, 979751, +, 894, 101396; cds-WP_000899600.1, NZ_CP025268.1, 979728, 980507, -, 780, 0; cds-WP_001298300.1, NZ_CP025268.1, 980643, 981428, +, 786, 58179; cds-WP_001288850.1, NZ_CP025268.1, 981425, 982747, +, 1323, 563258; cds-WP_001295347.1, NZ_CP025268.1, 982728, 983432, +, 705, 499093; cds-WP_000572698.1, NZ_CP025268.1, 983432, 987892, +, 4461, 1463254; cds-WP_000925969.1, NZ_CP025268.1, 988153, 990000, +, 1848, 50; cds-WP_001295932.1, NZ_CP025268.1, 990181, 990729, +, 549, 1369; cds-WP_001109486.1, NZ_CP025268.1, 990756, 991403, +, 648, 129997; cds-WP_000462687.1, NZ_CP025268.1, 991625, 992815, -, 1191, 434944; cds-WP_000977920.1, NZ_CP025268.1, 993000, 994088, -, 1089, 16802; cds-WP_000117881.1, NZ_CP025268.1, 994691, 996091, -, 1401, 621626; cds-WP_001307697.1, NZ_CP025268.1, 996260, 997462, -, 1203, 86598; cds-WP_000193841.1, NZ_CP025268.1, 997728, 1000340, +, 2613, 170974; cds-WP_001090506.1, NZ_CP025268.1, 1000383, 1001150, -, 768, 4; cds-WP_000235203.1, NZ_CP025268.1, 1001147, 1001938, -, 792, 0; cds-WP_000056006.1, NZ_CP025268.1, 1001949, 1003094, -, 1146, 0; cds-WP_001244308.1, NZ_CP025268.1, 1003091, 1004050, -, 960, 0; cds-WP_001263933.1, NZ_CP025268.1, 1004043, 1004618, -, 576, 0; cds-WP_000750293.1, NZ_CP025268.1, 1004974, 1005513, +, 540, 0; cds-WP_001295349.1, NZ_CP025268.1, 1005596, 1006297, +, 702, 0; cds-WP_000286312.1, NZ_CP025268.1, 1006322, 1008922, +, 2601, 6; cds-WP_001165668.1, NZ_CP025268.1, 1008913, 1009983, +, 1071, 0; cds-WP_000730614.1, NZ_CP025268.1, 1009995, 1010537, +, 543, 0; cds-WP_000919497.1, NZ_CP025268.1, 1010545, 1011060, +, 516, 0; cds-WP_001111465.1, NZ_CP025268.1, 1011026, 1011763, +, 738, 0; cds-WP_001295352.1, NZ_CP025268.1, 1011874, 1012884, +, 1011, 4337; cds-CXP41_RS05255, NZ_CP025268.1, 1013058, 1013599, +, 542, 900; cds-WP_000212426.1, NZ_CP025268.1, 1013596, 1014705, -, 1110, 778; cds-WP_001086539.1, NZ_CP025268.1, 1014949, 1017057, +, 2109, 966096; cds-WP_000053099.1, NZ_CP025268.1, 1017069, 1018976, +, 1908, 503501; cds-WP_000333176.1, NZ_CP025268.1, 1019106, 1020359, +, 1254, 136698; cds-WP_000445533.1, NZ_CP025268.1, 1020364, 1022004, +, 1641, 546554; cds-WP_000759120.1, NZ_CP025268.1, 1022001, 1022564, +, 564, 157996; cds-WP_000828648.1, NZ_CP025268.1, 1022820, 1022987, +, 168, 13088; cds-WP_000227927.1, NZ_CP025268.1, 1023057, 1023575, -, 519, 43952; cds-WP_000156518.1, NZ_CP025268.1, 1023644, 1025404, -, 1761, 396922; cds-WP_000877161.1, NZ_CP025268.1, 1025590, 1026042, +, 453, 374; cds-WP_000750416.1, NZ_CP025268.1, 1026118, 1027158, -, 1041, 4387920; cds-WP_000288710.1, NZ_CP025268.1, 1027515, 1028024, -, 510, 10; cds-WP_000839153.1, NZ_CP025268.1, 1028243, 1028872, +, 630, 33881; cds-WP_000875061.1, NZ_CP025268.1, 1028835, 1030997, -, 2163, 5491; cds-WP_001261235.1, NZ_CP025268.1, 1031007, 1031453, -, 447, 3521; cds-WP_001295354.1, NZ_CP025268.1, 1031576, 1033630, +, 2055, 2845; cds-WP_000424181.1, NZ_CP025268.1, 1033662, 1034120, -, 459, 49; cds-WP_000847791.1, NZ_CP025268.1, 1034216, 1034878, -, 663, 0; cds-WP_001301418.1, NZ_CP025268.1, 1035051, 1035464, +, 414, 0; cds-WP_001295356.1, NZ _CP025268.1, 1035509, 1035826, -, 318, 7846; cds-WP_000116297.1, NZ_CP025268.1, 1035884, 1037074, -, 1191, 142701; cds-WP_000048252.1, NZ_CP025268.1, 1037169, 1037447, +, 279, 0; cds-WP_000904442.1, NZ_CP025268.1, 1037444, 1037773, -, 330, 1; cds-WP_000375136.1, NZ_CP025268.1, 1037864, 1038523, -, 660, 10041; cds-WP_001058323.1, NZ_CP025268.1, 1039244, 1040362, +, 1119, 0; cds-WP_000107384.1, NZ_CP025268.1, 1040359, 1042152, +, 1794, 0; cds-WP_001186424.1, NZ_CP025268.1, 1042171, 1042878, +, 708, 32553; cds-WP_000003671.1, NZ_CP025268.1, 1042875, 1043462, +, 588, 81173; cds-WP_000063978.1, NZ_CP025268.1, 1043459, 1043857, +, 399, 0; cds-WP_000004899.1, NZ_CP025268.1, 1043854, 1044711, +, 858, 0; cds-WP_000263582.1, NZ_CP025268.1, 1044845, 1046389, +, 1545, 111; cds-WP_000460803.1, NZ_CP025268.1, 1046401, 1047537, +, 1137, 0; cds-WP_000270305.1, NZ_CP025268.1, 1047550, 1047642, +, 93, 0; cds-WP_001300464.1, NZ_CP025268.1, 1047722, 1049020, +, 1299, 0; cds-WP_000208650.1, NZ_CP025268.1, 1049135, 1051315, -,
2181, 21531; cds-WP_000057871.1, NZ_CP025268.1, 1051335, 1051781, -, 447, 0; cds-WP_001295357.1, NZ_CP025268.1, 1051769, 1052908, -, 1140, 0; cds-WP_000742328.1, NZ_CP025268.1, 1052954, 1055050, -, 2097, 0; cds-WP_001038079.1, NZ_CP025268.1, 1055050, 1055796, -, 747, 0; cds-WP_001247610.1, NZ_CP025268.1, 1055793, 1056437, -, 645, 87; cds-WP_001295358.1, NZ_CP025268.1, 1056544, 1056849, -, 306, 0; cds-CXP41_RS05480, NZ_CP025268.1, 1056938, 1057634, +, 697, 135520; cds-WP_000087763.1, NZ_CP025268.1, 1058067, 1058279, -, 213, 7403; cds-WP_000066490.1, NZ_CP025268.1, 1058565, 1058777, +, 213, 108600; cds-WP_071524879.1, NZ_CP025268.1, 1058788, 1058976, +, 189, 41729; cds-WP_001309400.1, NZ _CP025268.1, 1058951, 1059181, +, 231, 28719; cds-WP_001019197.1, NZ _CP025268.1, 1059171, 1059344, +, 174, 44619; cds-WP_000829662.1, NZ_CP025268.1, 1059393, 1060466, -, 1074, 783; cds-WP_001300633.1, NZ_CP025268.1, 1060538, 1063282, -, 2745, 10299; cds-WP_001264933.1, NZ_CP025268.1, 1063365, 1064393, +, 1029, 0; cds-WP_001120125.1, NZ_CP025268.1, 1064366, 1065058, -, 693, 0; cds-WP_001230242.1, NZ_CP025268.1, 1065188, 1066360, +, 1173, 0; cds-WP_001062091.1, NZ_CP025268.1, 1066360, 1068906, +, 2547, 247; cds-WP_000209894.1, NZ_CP025268.1, 1068903, 1069502, +, 600, 5; cds-WP_000024560.1, NZ_CP025268.1, 1069654, 1069959, -, 306, 1; cds-WP 000420621.1, NZ_CP025268.1, 1069959, 1070879, -, 921, 0; cds-WP_000535346.1, NZ_CP025268.1, 1071140, 1072396, +, 1257, 0; cds-WP_001044279.1, NZ_CP025268.1, 1072689, 1073930, +, 1242, 22; cds-WP_001143120.1, NZ_CP025268.1, 1073968, 1074195, -, 228, 0; cds-WP_001151437.1, NZ_CP025268.1, 1074216, 1074812, -, 597, 0; cds-WP_001273658.1, NZ_CP025268.1, 1075185, 1075358, +, 174, 0; cds-WP_001326838.1, NZ_CP025268.1, 1075613, 1076941, -, 1329, 0; cds-WP_001028083.1, NZ_CP025268.1, 1076962, 1077456, -, 495, 0; cds-WP_001001176.1, NZ_CP025268.1, 1077467, 1078057, -, 591, 0; cds-WP_000777653.1, NZ_CP025268.1, 1078067, 1078867, -, 801, 0; cds-WP_001126780.1, NZ_CP025268.1, 1078875, 1079261, -, 387, 0; cds-WP_001393558.1, NZ_CP025268.1, 1079273, 1079965, -, 693, 0; cds-WP_155555868.1, NZ_CP025268.1, 1079965, 1081056, -, 1092, 0; cds-WP_000191701.1, NZ_CP025268.1, 1081344, 1081982, +, 639, 0; cds-WP_001326840.1, NZ _CP025268.1, 1082022, 1085984, -, 3963, 177; cds-WP_000979516.1, NZ _CP025268.1, 1086039, 1086248, +, 210, 0; cds-WP_001018479.1, NZ_CP025268.1, 1086407, 1087915, +, 1509, 18210; cds-CXP41_RS05660, NZ_CP025268.1, 1088458, 1089287, +, 830, 169080; cds-WP_000154398.1, NZ_CP025268.1, 1089345, 1090472, +, 1128, 2961; cds-WP_001199471.1, NZ_CP025268.1, 1090478, 1091749, +, 1272, 24; cds-WP_000533522.1, NZ_CP025268.1, 1092370, 1093158, +, 789, 0; cds-WP_001061095.1, NZ_CP025268.1, 1093208, 1093621, -, 414, 0; cds-WP_000610451.1, NZ_CP025268.1, 1093623, 1094948, -, 1326, 0; cds-WP_000945561.1, NZ_CP025268.1, 1094941, 1096959, -, 2019, 20167; cds-WP_000287458.1, NZ_CP025268.1, 1096968, 1099391, -, 2424, 0; cds-WP_000409873.1, NZ_CP025268.1, 1099978, 1101336, +, 1359, 0; cds-WP_085947771.1-3, NZ_CP025268.1;NZ_CP025268.1, 1101377;1102278, 1102278;1102539, -;-, 1163, 3; cds-CXP41_RS05710, NZ_CP025268.1, 1102607, 1102948, +, 342, 0; cds-WP_000611859.1, NZ _CP025268.1, 1102945, 1103931, +, 987, 0; cds-WP_000351317.1, NZ_CP025268.1, 1104988, 1105926, +, 939, 8; cds-WP_000283667.1, NZ_CP025268.1, 1105981, 1106718, +, 738, 73159; cds-WP_001001921.1, NZ_CP025268.1, 1106742, 1107296, +, 555, 86492; cds-WP _001300785.1, NZ_CP025268.1, 1107398, 1107889, +, 492, 29; cds-WP_001189321.1, NZ_CP025268.1, 1107953, 1108786, -, 834, 58; cds-WP_001264088.1, NZ_CP025268.1, 1108813, 1109229, -, 417, 0; cds-WP_000833288.1, NZ_CP025268.1, 1109254, 1109643, -, 390, 0; cds-WP_000481509.1, NZ_CP025268.1, 1109648, 1110298, -, 651, 0; cds-WP_000791650.1, NZ_CP025268.1, 1111053, 1111508, +, 456, 0; cds-WP_000771437.1, NZ_CP025268.1, 1111549, 1112004, +, 456, 22; cds-WP_001094300.1, NZ_CP025268.1, 1112063, 1112395, +, 333, 9; cds-WP_000489587.1, NZ_CP025268.1, 1112516, 1112827, +, 312, 0; cds-WP_000857405.1, NZ_CP025268.1, 1112922, 1113455, +, 534, 26; cds-WP_001307714.1, NZ_CP025268.1, 1113397, 1114878, +, 1482, 250912; cds-WP_001070375.1, NZ_CP025268.1, 1114886, 1116043, -, 1158, 52; cds-WP_001343212.1, NZ_CP025268.1, 1116437, 1117972, +, 1536, 298536; cds-WP_001295445.1, NZ_CP025268.1, 1117965, 1120508, +, 2544, 2026219; cds-WP_001237205.1, NZ_CP025268.1, 1120681, 1120908, +, 228, 51214; cds-WP_000180056.1, NZ_CP025268.1, 1120909, 1121283, -, 375, 2; cds-WP_000074172.1, NZ_CP025268.1, 1121366, 1122592, -, 1227, 18308; cds-WP_000183364.1, NZ_CP025268.1, 1122764, 1123684, -, 921, 15863; cds-WP_001144615.1, NZ_CP025268.1, 1123909, 1124961, +, 1053, 20849; cds-WP_000749261.1, NZ_CP025268.1, 1125003, 1125578, -, 576, 84476; cds-WP_000011114.1, NZ_CP025268.1, 1125582, 1126148, -, 567, 0; cds-WP_001253418.1, NZ_CP025268.1, 1126409, 1126522, -, 114, 0; cds-WP_000872833.1, NZ_CP025268.1, 1126570, 1127688, -, 1119, 14; cds-WP_000414438.1, NZ_CP025268.1, 1127803, 1128057, -, 255, 0; cds-WP_001217754.1, NZ_CP025268.1, 1128344, 1128589, -, 246, 0; cds-WP_000126534.1, NZ_CP025268.1, 1128663, 1129709, -, 1047, 2289; cds-WP_001295443.1, NZ_CP025268.1, 1129815, 1130375, -, 561, 115; cds-WP_000780912.1, NZ_CP025268.1, 1130509, 1131156, -, 648, 43202; cds-WP_101348576.1, NZ_CP025268.1, 1131220, 1132428, -, 1209, 0; cds-WP_000468186.1, NZ_CP025268.1, 1132664, 1133248, +, 585, 0; cds-WP_000877107.1, NZ_CP025268.1, 1133259, 1133906, +, 648, 1214; cds-WP_000736158.1, NZ_CP025268.1, 1133908, 1134831, +, 924, 72; cds-WP_001050683.1, NZ_CP025268.1, 1134941, 1136476, +, 1536, 29054; cds-WP_000197363.1, NZ_CP025268.1, 1136516, 1136932, -, 417, 223329; cds-WP_000020880.1, NZ_CP025268.1, 1136937, 1137230, -, 294, 76178; cds-WP_000905458.1, NZ_CP025268.1, 1137306, 1137965, -, 660, 6383; cds-WP_000884702.1, NZ_CP025268.1, 1138120, 1138536, +, 417, 47522; cds-WP_001196460.1, NZ_CP025268.1, 1138540, 1138944, +, 405, 146912; cds-WP_000020486.1, NZ_CP025268.1, 1138956, 1139651, +, 696, 1076371; cds-WP_000885873.1, NZ_CP025268.1, 1139676, 1140884, +, 1209, 1124646; cds-WP_000349316.1, NZ_CP025268.1, 1140904, 1141659, +, 756, 277131; cds-WP_000625837.1, NZ_CP025268.1, 1141831, 1142613, +, 783, 526748; cds-WP_001295442.1, NZ_CP025268.1, 1142666, 1143364, +, 699, 101817; cds-WP_000589326.1, NZ_CP025268.1, 1143376, 1144473, +, 1098, 163847; cds-WP_001295441.1, NZ_CP025268.1, 1144473, 1145414, +, 942, 34129; cds-CXP41_RS06000, NZ_CP025268.1, 1145480, 1147122, +, 1643, 469683; cds-WP_001212782.1, NZ_CP025268.1, 1147134, 1148087, +, 954, 220104; cds-WP_000827360.1, NZ_CP025268.1, 1148283, 1151468, -, 3186, 960861; cds-WP_000846343.1, NZ_CP025268.1, 1152041, 1153000, +, 960, 45946; cds-WP_001125202.1, NZ_CP025268.1, 1153112, 1153696, -, 585, 88622; cds-WP_001174481.1, NZ_CP025268.1, 1153895, 1154416, +, 522, 1435619; cds-WP_000290727.1, NZ_CP025268.1, 1154468, 1154641, +, 174, 679039; cds-WP_000197597.1, NZ_CP025268.1, 1154722, 1155792, +, 1071, 1205000; cds-WP_000288132.1, NZ_CP025268.1, 1155860, 1156813, +, 954, 3480344; cds-WP_000191372.1, NZ_CP025268.1, 1156829, 1157758, +, 930, 938806; cds-WP_001008535.1, NZ_CP025268.1, 1157771, 1158505, +, 735, 1538109; cds-WP_000103754.1, NZ_CP025268.1, 1158716, 1158952, +, 237, 996475; cds-WP_101348577.1, NZ_CP025268.1, 1159040, 1160281, +, 1242, 2687278; cds-WP_000478681.1, NZ_CP025268.1, 1160401, 1161210, +, 810, 199189; cds-WP_000756827.1, NZ_CP025268.1, 1161213, 1162235, +, 1023, 205660; cds-WP_001257000.1, NZ_CP025268.1, 1162225, 1162866, +, 642, 105026; cds-WP_001267956.1, NZ_CP025268.1, 1162863, 1163867, +, 1005, 63528; cds-WP_000480245.1, NZ_CP025268.1, 1163878, 1164675, +, 798, 80914; cds-WP_000475719.1, NZ_CP025268.1, 1164970, 1166403, +, 1434, 883967; cds-WP_000953441.1, NZ_CP025268.1, 1166463, 1168652, -, 2190, 197; cds-WP_000807125.1, NZ_CP025268.1, 1168986, 1169345, +, 360, 1315; cds-WP_001225295.1, NZ _CP025268.1, 1169348, 1169725, +, 378, 34; cds-WP_000164439.1, NZ_CP025268.1, 1169739, 1170380, +, 642, 18; cds-WP_001116592.1, NZ_CP025268.1, 1170361, 1171185, +, 825, 1561; cds-WP_000529320.1, NZ_CP025268.1, 1171196, 1172221, +, 1026, 45029; cds-WP_000587933.1, NZ_CP025268.1, 1172244, 1172786, +, 543, 63224; cds-WP_000211045.1, NZ_CP025268.1, 1173186, 1174490, +, 1305, 765029; cds-WP_001043459.1, NZ_CP025268.1, 1174700, 1175239, +, 540, 39204; cds-CXP41_RS06145, NZ_CP025268.1, 1175301, 1175932, -, 632, 1; cds-WP_000800153.1, NZ_CP025268.1, 1176173, 1176430, +, 258, 0; cds-WP_001350500.1, NZ_CP025268.1, 1176512, 1177474, -, 963, 3; cds-WP_001115094.1, NZ_CP025268.1, 1177618, 1181064, -, 3447, 130329; cds-WP_000810253.1, NZ_CP025268.1, 1181192, 1182265, -, 1074, 0; cds-WP_000284714.1, NZ_CP025268.1, 1182527, 1183726, +, 1200, 32890; cds-WP_001033694.1, NZ_CP025268.1, 1183719, 1184420, +, 702, 5; cds-WP_001251348.1, NZ_CP025268.1, 1184420, 1185664, +, 1245, 125905; cds-WP_000291270.1, NZ _CP025268.1, 1185693, 1186604, +, 912, 146433; cds-WP_000952737.1, NZ_CP025268.1, 1186620, 1187459, +, 840, 326778; cds-WP_000713462.1, NZ_CP025268.1, 1187579, 1188367, -, 789, 123643; cds-WP_000076376.1, NZ_CP025268.1, 1188364, 1188825, -, 462, 21043; cds-WP_000759317.1, NZ_CP025268.1, 1188883, 1189929, -, 1047, 648491; cds-WP_000580316.1, NZ_CP025268.1, 1189926, 1190720, -, 795, 417457; cds-WP_ 000799401.1, NZ_CP025268.1, 1190717, 1191574, -, 858, 265718; cds-WP_000531594.1, NZ_CP025268.1, 1191558, 1192694, -, 1137, 601018; cds-WP_000359434.1, NZ_CP025268.1, 1192944, 1194170, +, 1227, 6063; cds-WP_000456506.1, NZ_CP025268.1, 1194219, 1195340, -, 1122, 125915; cds-WP_000735412.1, NZ_CP025268.1, 1195416, 1196876, -, 1461, 152056; cds-WP_001265471.1, NZ_CP025268.1, 1196876, 1197547, -, 672, 41145; cds-WP_000423742.1, NZ_CP025268.1, 1197716, 1199086, -, 1371, 228076; cds-WP_ 001297479.1, NZ_CP025268.1, 1199090, 1199731, -, 642, 220776; cds-WP_001297484.1, NZ_CP025268.1, 1199767, 1200873, -, 1107, 320405; cds-WP_000476093.1, NZ_CP025268.1, 1200927, 1201388, -, 462, 112809; cds-WP_001248691.1, NZ_CP025268.1, 1201398, 1202051, -, 654, 18625; cds-WP_000444484.1, NZ_CP025268.1, 1202223, 1203473, +, 1251, 466826; cds-WP_000241967.1, NZ_CP025268.1, 1203967, 1204632, -, 666, 39487; cds-WP_010723094.1, NZ_CP025268.1, 1204633, 1205337, -, 705, 4; cds-WP_001257372.1, NZ_CP025268.1, 1205795, 1206688, +, 894, 94; cds-CXP41_RS06295, NZ_CP025268.1, 1206779, 1207907, -, 1129, 22; cds-WP_101348578.1, NZ_CP025268.1, 1207888, 1208133, -, 246, 0; cds-WP_011443584.1, NZ_CP025268.1, 1208170, 1208481, -, 312, 4; cds-WP_010723095.1, NZ_CP025268.1, 1208598, 1208939, +, 342, 0; cds-WP_001005353.1, NZ_CP025268.1, 1208877, 1209185, -, 309, 0; cds-WP_000848748.1, NZ_CP025268.1, 1209360, 1210034, -, 675, 25729; cds-WP_000649480.1, NZ_CP025268.1, 1210125, 1210325, +, 201, 0; cds-WP_000515837.1, NZ_CP025268.1, 1210369, 1210926, +, 558, 0; cds-WP_101348579.1, NZ_CP025268.1, 1211102, 1211281, +, 180, 0; cds-WP_000104929.1, NZ_CP025268.1, 1211271, 1212638, +, 1368, 0; cds-WP_000605606.1, NZ_CP025268.1, 1212650, 1212832, +, 183, 0; cds-CXP41_RS06355, NZ_CP025268.1, 1212832, 1213239, +, 408, 0; cds-WP_001350503.1, NZ_CP025268.1, 1213232, 1214023, +, 792, 0; cds-WP_000383574.1, NZ_CP025268.1, 1214014, 1214598, +, 585, 0; cds-WP_000554703.1, NZ_CP025268.1, 1214602, 1215231, +, 630, 0; cds-CXP41_RS06375, NZ_CP025268.1, 1215233, 1215637, +, 405, 0; cds-CXP41_RS06380, NZ_CP025268.1, 1215635, 1216219, -, 585, 0; cds-WP_024184299.1, NZ_CP025268.1, 1216219, 1216713, -, 495, 0; cds-WP_000905001.1, NZ_CP025268.1, 1216785, 1217339, +, 555, 0; cds-WP_000557907.1, NZ_CP025268.1, 1217446, 1218279, +, 834, 0; cds-CXP41_RS06400, NZ_CP025268.1, 1218510, 1218677, +, 168, 0; cds-WP_001295666.1, NZ_CP025268.1, 1218780, 1219103, -, 324, 0; cds-WP_120795380.1, NZ_CP025268.1, 1219360, 1219404, -, 45, 0; cds-WP_032082692.1, NZ_CP025268.1, 1219640, 1219750, -, 111, 0; cds-WP_ 000373101.1, NZ_CP025268.1, 1219803, 1220207, -, 405, 0; cds-WP_000332303.1, NZ_CP025268.1, 1220428, 1221159, -, 732, 4; cds-WP_001299269.1, NZ_CP025268.1, 1221364, 1222575, -, 1212, 79766; cds-WP_000554140.1, NZ_CP025268.1, 1222889, 1223125, +, 237, 0; cds-WP_000858002.1, NZ_CP025268.1, 1223168, 1223440, +, 273, 0; cds-WP_000888772.1, NZ_CP025268.1, 1223469, 1223735, +, 267, 0; cds-WP_001065752.1, NZ_CP025268.1, 1223848, 1224096, +, 249, 0; cds-WP_001246499.1, NZ_CP025268.1, 1224426, 1225949, +, 1524, 161; cds-
WP_001065861.1, NZ_CP025268.1, 1226081, 1226299, +, 219, 1; cds-CXP41_RS24365, NZ_CP025268.1, 1226699, 1229346, +, 2648, 1; cds-WP_000979977.1, NZ_CP025268.1 1229403, 1229738, -, 336, 0; cds-WP_000726974.1, NZ_CP025268.1, 1229742, 1230086, -, 345, 0; cds-WP_000122462.1, NZ_CP025268.1 1230088, 1230261, -, 174, 0; cds-WP_001325005.1, NZ_CP025268.1 1230362, 1230547, +, 186, 70; cds-WP_001131446.1, NZ_CP025268.1, 1230508, 1230627, +, 120, 0; cds-CXP41_RSO6490, NZ_CP025268.1, 1230662, 1231005, +, 344, 0; cds-WP_001185665.1, NZ_CP025268.1 CP025268.1, 1231377, 1231643, -, 267, 336; cds-WP_000101055.1, NZ_CP025268.1 1231647, 1232459, -, 813, 559687; cds-WP_001301105.1, NZ_CP025268.1, 1232483, 1233178, -, 696, 32889; cds-WP_0010568401, NZ_CP025268.1, 1233698, 1234066, +, 369, 0; cds-WP_000695215.1, NZ_CP025268.1, 1234169, 1234570, -, 402, 0; cds-WP_001295992.1, NZ_CP025268.1, 1234812, 1235105, +, 294, 0; cds-WP_101348580.1, NZ_CP025268.1, 1235177, 1235836, +, 660, 3756; cds-WP_001306825.1, NZ_CP025268.1, 1235928, 1236374, +, 447, 1; cds-WP_001336523.1, NZ_CP025268.1, 1236581, 1237492, -, 912, 0; cds-WP_000897378.1, NZ_CP025268.1, 1237865, 1238284, +, 420, 0; cds-WP_000457616.1, NZ_CP025268.1, 1238284, 1239552, +, 1269, 23344; cds-WP_000943459.1, NZ_CP025268.1, 1239598, 1240128, -, 531, 7340; cds-WP_000406391.1, NZ_CP025268.1, 1240274, 1241815, -, 1542, 159507; cds-WP_000234823.1, NZ_CP025268.1, 1242036, 1242755, +, 720, 220; cds-WP_000190854.1, NZ_CP025268.1, 1242807, 1244339, -, 1533, 1431; cds-WP_001266908.1, NZ_CP025268.1, 1244669, 1245967, +, 1299, 290; cds-WP_000197881.1, NZ_CP025268.1, 1245977, 1247047, +, 1071, 3701; cds-WP_000340194.1, NZ_CP025268.1, 1247433, 1249169, -, 1737, 50; cds-WP_000051560.1, NZ_CP025268.1, 1249264, 1250178, -, 915, 8; cds-WP_001301104.1, NZ_CP025268.1, 1250278, 1250889, +, 612, 6999; cds-WP_000020169.1, NZ_CP025268.1, 1250891, 1251625, -, 735, 36475; cds-WP_001310727.1, NZ_CP025268.1, 1251826, 1252080, +, 255, 0; cds-WP_000615067.1, NZ_CP025268.1, 1252258, 1252698, +, 441, 0; cds-WP_000841714.1, NZ_CP025268.1, 1252777, 1254474, -, 1698, 8228; cds-WP_001301101.1, NZ_CP025268.1, 1254794, 1256212, -, 1419, 13484; cds-WP_000059411.1, NZ_CP025268.1, 1256223, 1256855, -, 633, 0; cds-CXP41_RS06650, NZ_CP025268.1, 1256866, 1257937, -, 1072, 0; cds-WP _101348581.1, NZ_CP025268.1, 1258165, 1260084, +, 1920, 1807; cds-WP_001334788.1, NZ_CP025268.1, 1260184, 1263051, -, 2868, 3; cds-WP_000505866.1, NZ_CP025268.1, 1263820, 1264911, -, 1092, 333168; cds-WP_000152933.1, NZ_CP025268.1, 1265028, 1265612, -, 585, 7625; cds-WP_000823885.1, NZ_CP025268.1, 1265890, 1266168, +, 279, 3740; cds-WP_001033352.1, NZ_CP025268.1, 1266223, 1267902, -, 1680, 161138; cds-WP_001298109.1, NZ_CP025268.1, 1268027, 1268974, -, 948, 112829; cds-WP_001260332.1, NZ_CP025268.1, 1269125, 1269976, -, 852, 19540; cds-WP_001130692.1, NZ_CP025268.1, 1269976, 1270599, -, 624, 62029; cds-WP_001299679.1, NZ_CP025268.1, 1270813, 1272069, +, 1257, 9288; cds-WP_000456467.1, NZ_CP025268.1, 1272111, 1272944, +, 834, 99296; cds-WP_000200374.1, NZ_CP025268.1, 1272941, 1273333, +, 393, 217940; cds-WP_001257044.1, NZ_CP025268.1, 1273337, 1274146, +, 810, 419626; cds-WP_000811065.1, NZ_CP025268.1, 1274182, 1275036, +, 855, 47500; cds-WP_000170955.1, NZ_CP025268.1, 1275185, 1275292, -, 108, 3934; cds-WP_000170963.1, NZ_CP025268.1, 1275720, 1275827, -, 108, 550; cds-WP_000170955.1-2, NZ_CP025268.1, 1276255, 1276362, -, 108, 1678; cds-WP_101348582.1, NZ_CP025268.1, 1276766, 1277866, -, 1101, 9660; cds-WP _001146444.1, NZ_CP025268.1, 1278136, 1278366, +, 231, 0; cds-WP_001336325.1, NZ_CP025268.1, 1278524, 1279219, +, 696, 0; cds-WP_001169669.1, NZ_CP025268.1, 1279263, 1279616, -, 354, 0; cds-WP_000086217.1, NZ_CP025268.1, 1279801, 1281195, +, 1395, 1078; cds-WP_000070491.1, NZ_CP025268.1, 1281196, 1281846, -, 651, 274; cds-WP_000918073.1, NZ_CP025268.1, 1281839, 1283635, -, 1797, 87229; cds-WP_000019827.1, NZ_CP025268.1, 1283974, 1285365, +, 1392, 0; cds-WP_000032939.1, NZ_CP025268.1, 1285881, 1289624, +, 3744, 0; cds-WP_000702650.1, NZ_CP025268.1, 1289621, 1291159, +, 1539, 0; cds-WP_000571681.1, NZ_CP025268.1, 1291156, 1291866, +, 711, 0; cds-WP_001160108.1, NZ_CP025268.1, 1291866, 1292543, +, 678, 0; cds-WP_000555857.1, NZ_CP025268.1, 1293799, 1294641, -, 843, 144106; cds-WP_001307143.1, NZ_CP025268.1, 1294691, 1295149, -, 459, 524; cds-WP_001295622.1, NZ_CP025268.1, 1295262, 1296167, +, 906, 1571; cds-WP_000193447.1, NZ_CP025268.1, 1296259, 1297272, +, 1014, 298; cds-WP_000718995.1, NZ _CP025268.1, 1297474, 1298382, +, 909, 242776; cds-WP_001287378.1, NZ_CP025268.1, 1298526, 1298939, -, 414, 189818; cds-WP_000068077.1, NZ_CP025268.1, 1299544, 1300161, +, 618, 1; cds-CXP41_RS06895, NZ_CP025268.1, 1300443, 1301339, -, 897, 101136; cds-WP_000301651.1, NZ_CP025268.1, 1301463, 1304138, -, 2676, 544626; cds-WP_211180492.1, NZ_CP025268.1, 1304531, 1304584, +, 54, 0; cds-WP_000616554.1, NZ_CP025268.1, 1304615, 1305262, +, 648, 1; cds-WP_001211520.1, NZ_CP025268.1, 1305420, 1305716, -, 297, 11217; cds-WP_001393463.1, NZ_CP025268.1, 1306000, 1307631, +, 1632, 2397957; cds-WP_000911112.1, NZ_CP025268.1, 1307717, 1308637, +, 921, 847228; cds-WP_000979661.1, NZ_CP025268.1, 1308652, 1309560, +, 909, 653115; cds-WP_000110948.1, NZ_CP025268.1, 1309572, 1310585, +, 1014, 765740; cds-WP_000994906.1, NZ_CP025268.1 1310582, 1311586, +, 1005, 263125; cds-WP_000366959.1, NZ_CP025268.1 1311639, 1311968, -, 330, 16994; cds-WP_000214516.1, NZ_CP025268.1, 1312003, 1313463, -, 1461, 73962; cds-WP_001309467.1, NZ_CP025268.1, 1313606, 1313779, +, 174, 0; cds-WP_001295624.1, NZ_CP025268.1, 1313834, 1315087, -, 1254, 2; cds-WP_000967595.1, NZ_CP025268.1, 1315387, 1315683, -, 297, 0; cds-WP_001360141.1, NZ_CP025268.1, 1315907, 1316626, +, 720, 101510; cds-WP_000108160.1, NZ_CP025268.1, 1316666, 1317064, -, 399, 26993; cds-WP_000808667.1, NZ_CP025268.1, 1317708, -, 540, 2651; cds-WP_000028545.1, NZ_CP025268.1, 1317738, 1318481, -, 744, 2; cds-CXP41_RS24150, NZ_CP025268.1, 1318509, 1318598, -, 90, 0; cds-WP_000737226.1, NZ_CP025268.1, 1318838, 1319476, +, 639, 0; cds-WP_001079505.1, NZ_CP025268.1,1319536, 1320042, -, 507, 0; cds-WP_001056490.1, NZ_CP025268.1, 1320088, 1320588, -, 501, 147; cds-WP_000807651.1, NZ_CP025268.1, 1320674, 1320853, -, 180, 0; cds-WP_000443067.1, NZ_CP025268.1, 1321234, 1322040, -, 807, 193; cds-WP_000209520.1, NZ_CP025268.1, 1322040, 1323233, -, 1194, 26649; cds-WP_000983871.1, NZ_CP025268.1, 1323245, 1324606, -, 1362, 19; cds-WP_000763511.1, NZ_CP025268.1, 1324607, 1326202, -, 1596, 19; cds-WP_001194582.1, NZ_CP025268.1, 1326202, 1327764, -, 1563, 0; cds-WP_001700591.1, NZ_CP025268.1, 1327856, 1327900, -, 45, 0; cds-WP_001285661.1, NZ_CP025268.1, 1328038, 1328919, +, 882, 4; cds-WP_001295575.1, NZ_CP025268.1, 1328916, 1329536, +, 621, 728; cds-WP_001326916.1, NZ_CP025268.1, 1329564, 1331459, +, 1896, 1109; cds-WP_001291217.1, NZ_CP025268.1, 1331670, 1332545, +, 876, 223441; cds-WP_001278906.1, NZ_CP025268.1, 1332585, 1333175, -, 591, 65112; cds-WP_000559286.1, NZ_CP025268.1, 1333172, 1333930, -, 759, 46; cds-WP_000422045.1, NZ_CP025268.1, 1334150, 1335199, +, 1050, 10878; cds-WP_001031530.1, NZ_CP025268.1, 1335235, 1335486, -, 252, 19; cds-WP_001297122.1, NZ_CP025268.1, 1335866, 1338463, +, 2598, 1375391; cds-WP_000776253.1, NZ_CP025268.1, 1338673, 1339647, +, 975, 2188; cds-WP_001300896.1, NZ_CP025268.1, 1339966, 1340106, +, 141, 0; cds-WP_001297116.1, NZ_CP025268.1, 1340109, 1340276, +, 168, 3; cds-WP_001310756.1, NZ_CP025268.1, 1340390, 1340485, +, 96, 0; cds-WP_000099535.1, NZ_CP025268.1, 1340649, 1343324, +, 2676, 225194; cds-WP_001176295.1, NZ_CP025268.1, 1343388, 1343978, -, 591, 142527; cds-WP_001256538.1, NZ_CP025268.1, 1344148, 1344912, +, 765, 37919; cds-WP_000876286.1, NZ_CP025268.1, 1345061, 1345369, +, 309, 28; cds-WP_000891353.1, NZ_CP025268.1, 1345376, 1346545, +, 1170, 15236; cds-WP_000176270.1, NZ_CP025268.1, 1346739, 1347476, +, 738, 20754; cds-WP_001295580.1, NZ_CP025268.1, 1347476, 1347802, +, 327, 50902; cds-WP_000498253.1, NZ_CP025268.1, 1347928, 1348146, -, 219, 0; cds-WP_001088620.1, NZ_CP025268.1, 1348415, 1349164, -, 750, 16953; cds-WP_001288368.1, NZ_CP025268.1, 1349254, 1349427, -, 174, 63133; cds-WP_211180493.1, NZ_CP025268.1, 1349465, 1349593, +, 129, 32252; cds-WP_000859945.1, NZ_CP025268.1, 1349575, 1351560, -, 1986, 659935; cds-WP_000221855.1, NZ_CP025268.1, 1351614, 1351718, +, 105, 0; cds-WP_000484984.1, NZ_CP025268.1, 1351796, 1353730, -, 1935, 442405; cds-WP_001335988.1, NZ_CP025268.1,1353798, 1354925, -, 1128, 0; cds-WP_000506490.1, NZ_CP025268.1, 1355069, 1355857, -, 789, 177411; cds-WP_000565727.1, NZ_CP025268.1, 1356226, 1356579, -, 354, 1254; cds-WP_000573407.1, NZ_CP025268.1, 1356647, 1357453, -, 807, 10412; cds-WP_001128858.1, NZ_CP025268.1, 1357455, 1358447, -, 993, 2670; cds-WP_101348583.1, NZ_CP025268.1, 1358447, 1359337, -, 891, 54721; cds-WP_000583277.1, NZ_CP025268.1, 1359324, 1360289, -, 966, 540; cds-WP_001250216.1, NZ_CP025268.1, 1360286, 1361929, -, 1644, 68895; cds-WP_001015110.1, NZ_CP025268.1, 1362242, 1362487, -, 246, 2728; cds-WP_000996856.1, NZ_CP025268.1, 1362621, 1364006, -, 1386, 0; cds-WP_120795382.1, NZ_CP025268.1, 1363996, 1364160, -, 165, 0; cds-WP_001296746.1, NZ_CP025268.1, 1364309, 1365727, -, 1419, 5; cds-WP_001300506.1, NZ_CP025268.1, 1365939, 1366703, +, 765, 2995; cds-WP_001278727.1, NZ_CP025268.1, 1366730, 1367287, +, 558, 105443; cds-WP_001009090.1, NZ _CP025268.1, 1367562, 1369049, +, 1488, 0; cds-WP_000134870.1, NZ_CP025268.1, 1369051, 1370331, +, 1281, 0; cds-WP_000069229.1, NZ_CP025268.1, 1370369, 1371634, +, 1266, 0; cds-WP_001301108.1, NZ_CP025268.1, 1371754, 1372731, -, 978, 0; cds-WP_000511025.1, NZ_CP025268.1, 1372898, 1373566, +, 669, 1; cds-WP_001274963.1, NZ _CP025268.1, 1373620, 1373844, +, 225, 0; cds-WP_000907387.1, NZ_CP025268.1, 1373844, 1374203, +, 360, 0; cds-WP_001295585.1, NZ_CP025268.1, 1374212, 1374433, +, 222, 3; cds-WP_000473109.1, NZ_CP025268.1, 1374508, 1374822, +, 315, 0; cds-WP_000810499.1, NZ_CP025268.1, 1375034, 1376713, +, 1680, 7; cds-WP_000597466.1, NZ_CP025268.1, 1376727, 1378019, +, 1293, 0; cds-WP_001080790.1, NZ_CP025268.1, 1378040, 1378921, +, 882, 0; cds-WP_000224659.1, NZ_CP025268.1, 1378908, 1379750, +, 843, 0; cds-WP_000737347.1, NZ_CP025268.1,1379781, 1380833, +, 1053, 0; cds-WP_000690229.1, NZ_CP025268.1, 1380852, 1381640, +, 789, 0; cds-WP_001395397.1, NZ _CP025268.1, 1381650, 1382705, +, 1056, 0; cds-WP_000198036.1, NZ_CP025268.1, 1382702, 1384969, +, 2268, 0; cds-WP_000775794.1, NZ_CP025268.1, 1384966, 1385625, +, 660, 0; cds-CXP41_RS07340, NZ_CP025268.1, 1385639, 1386720, +, 1082, 0; cds-WP_000735257.1, NZ_CP025268.1, 1386765, 1387670, +, 906, 0; cds-WP_000075375.1, NZ_CP025268.1, 1387781, 1388779, -, 999, 0; cds-WP_000825775.1, NZ_CP025268.1, 1388935, 1390332, +, 1398, 1; cds-WP_000138728.1, NZ_CP025268.1, 1390329, 1391390, +, 1062, 2; cds-WP_097766186.1, NZ_CP025268.1, 1391538, 1393079, +, 1542, 228381; cds-WP_000084387.1, NZ_CP025268.1, 1393123, 1393629, -, 507, 157475; cds-CXP41_RS07375, NZ_CP025268.1, 1393748, 1394713, +, 966, 88144; cds-WP_101348584.1, NZ_CP025268.1 CP025268.1, 1394688, 1395416, -, 729, 44; cds-CXP41_RS07385, NZ_CP025268.1, 1395543, 1395680, -, 138, 109685; cds-WP_001300523.1, NZ_CP025268.1, 1395751, 1396671, -, 921, 61005; cds-WP_000817708.1, NZ_CP025268.1, 1396809, 1397708, +, 900, 939700; cds-WP_000683020.1, NZ_CP025268.1, 1398045, 1399658, +, 1614, 21905; cds-WP_000559900.1, NZ_CP025268.1, 1399709, 1400740, -, 1032, 190; cds-WP_000019403.1-4, NZ_CP025268.1, 1400930, 1401910, +, 981, 164925; cds-WP_000605090.1, NZ_CP025268.1, 1402183, 1402440, +, 258, 1854; cds-WP_001262123.1, NZ_CP025268.1, 1402490, 1403440, -, 951, 0; cds-WP_000611911.1, NZ_CP025268.1, 1404344, -, 753, 0; cds-CXP41_RS07430, NZ_CP025268.1, 1404539, 1405055, -, 517, 0; cds-WP_000062973.1, NZ_CP025268.1, 1405066, 1406592, -, 1527, 0; cds-WP_001156451.1, NZ_CP025268.1, 1406629, 1408074, -, 1446, 0; cds-WP_000444929.1, NZ_CP025268.1, 1408074, 1409384, -, 1311, 0; cds-WP_000885458.1, NZ_CP025268.1, 1409560, 1410468, +, 909, 0; cds-WP_001046829.1, NZ_CP025268.1, 1410798, 1411361, +, 564, 0; cds-WP_000628058.1, NZ_CP025268.1, 1411382, 1412614, -, 1233, 0; cds-WP_211180494.1, NZ_CP025268.1, 1412808, 1412855, +, 48, 0; cds-WP_000387388.1,
NZ_CP025268.1, 1412869, 1413852, +, 984, 0; cds-WP_000123737.1, NZ_CP025268.1, 1414330, 1415703, +, 1374, 0; cds-WP_001157406.1, NZ_CP025268.1, 1415832, 1416767, -, 936, 495142; cds-WP _000040852.1, NZ_CP025268.1, 1416819, 1418054, -, 1236, 121497; cds-WP_000079604.1, NZ_CP025268.1, 1418056, 1418271, -, 216, 0; cds-WP_000276809.1, NZ_CP025268.1, 1418350, 1418559, -, 210, 0; cds-WP_001317028.1, NZ_CP025268.1, 1418552, 1418746, -, 195, 0; cds-WP_000166319.1, NZ_CP025268.1, 1418803, 1419612, -, 810, 0; cds-WP_000105143.1, NZ_CP025268.1, 1419605, 1422205, -, 2601, 39; cds-WP_000632297.1, NZ_CP025268.1, 1422307, 1422582, -, 276, 0; cds-WP_001352098.1, NZ_CP025268.1, 1422657, 1422827, -, 171, 0; cds-WP_000560225.1, NZ_CP025268.1, 1422827, 1423048, -, 222, 0; cds-WP_001312793.1, NZ_CP025268.1, 1423490, 1423978, +, 489, 0; cds-WP_001169151.1, NZ_CP025268.1, 1423975, 1424130, -, 156, 5; cds-WP_000948459.1, NZ_CP025268.1, 1424584, 1425060, -, 477, 32423; cds-WP_000712069.1, NZ_CP025268.1, 1425184, 1425480, +, 297, 0; cds-WP_000693797.1, NZ_CP025268.1, 1425503, 1425925, +, 423, 0; cds-WP_000899748.1, NZ_CP025268.1, 1425938, 1426795, +, 858, 105; cds-WP_000788970.1, NZ_CP025268.1, 1426802, 1427548, +, 747, 0; cds-CXP41_RS07585, NZ_CP025268.1, 1427571, 1428023, +, 453, 0; cds-WP_001228696.1, NZ_CP025268.1, 1428219, 1428404, +, 186, 4458; cds-WP_001097895.1, NZ_CP025268.1, 1428601, 1430058, +, 1458, 89; cds-WP_001350510.1, NZ _CP025268.1, 1430196, 1430459, +, 264, 0; cds-WP_000091628.1, NZ_CP025268.1, 1430440, 1430799, +, 360, 0; cds-WP_001755909.1, NZ_CP025268.1, 1430907, 1431107, +, 201, 0; cds-CXP41_RS07620, NZ_CP025268.1, 1431273, 1432205, +, 933, 0; cds-CXP41_RS07625, NZ_CP025268.1, 1432208, 1432423, +, 216, 0; cds-WP_033544729.1, NZ_CP025268.1, 1432565, 1433545, -, 981, 5761; cds-CXP41_RS07640, NZ_CP025268.1, 1433615, 1433803, +, 189, 0; cds-WP_101348721.1, NZ_CP025268.1, 1433868, 1437230, +, 3363, 0; cds-WP_001698950.1, NZ_CP025268.1, 1437230, 1437805, +, 576, 0; cds-WP_000086527.1, NZ_CP025268.1, 1437903, 1438493, -, 591, 2; cds-WP_000836770.1, NZ_CP025268.1, 1438810, 1439043, -, 234, 288321; cds-WP_120795384.1, NZ_CP025268.1, 1439112, 1439225, -, 114, 119559; cds-WP_001157926.1, NZ_CP025268.1, 1439565, 1439738, +, 174, 3; cds-WP 001300461.1, NZ_CP025268.1, 1440004, 1440438, -, 435, 0; cds-WP_000837921.1, NZ_CP025268.1, 1440579, 1441712, -, 1134, 2; cds-WP_000628244.1, NZ_CP025268.1, 1442079, 1445603, -, 3525, 135076; cds-WP_001295715.1, NZ_CP025268.1, 1445877, 1446143, +, 267, 15; cds-WP_001298828.1, NZ_CP025268.1, 1446140, 1446562, -, 423, 7; cds-WP_000762236.1, NZ_CP025268.1, 1446673, 1447662, -, 990, 37307; cds-WP_000900941.1, NZ_CP025268.1, 1447870, 1450509, +, 2640, 263; cds-WP_000698145.1, NZ_CP025268.1, 1450506, 1450691, +, 186, 5665; cds-WP_001300734.1, NZ_CP025268.1, 1450699, 1451025, +, 327, 8510; cds-WP_001067514.1, NZ_CP025268.1, 1451197, 1452102, -, 906, 0; cds-WP_000138615.1, NZ_CP025268.1, 1452338, 1453837, +, 1500, 0; cds-WP_101348587.1, NZ_CP025268.1, 1453895, 1456168, -, 2274, 0; cds-WP_001186469.1, NZ_CP025268.1, 1456416, 1458461, -, 2046, 0; cds-WP_000191077.1, NZ_CP025268.1, 1458746, 1459675, +, 930, 0; cds-WP_000073393.1, NZ_CP025268.1, 1459687, 1459974, +, 288, 0; cds-WP_001072837.1, NZ_CP025268.1, 1459983, 1460729, +, 747, 0; cds-WP_001189201.1, NZ_CP025268.1, 1460744, 1461241, +, 498, 0; cds-WP_000206388.1, NZ_CP025268.1, 1461249, 1462319, +, 1071, 309; cds-WP_001292353.1, NZ_CP025268.1, 1462316, 1463083, +, 768, 0; cds-WP_000969784.1, NZ_CP025268.1, 1463083, 1463871, +, 789, 0; cds-WP_000973360.1, NZ_CP025268.1, 1463873, 1465300, +, 1428, 0; cds-WP_000018413.1, NZ_CP025268.1, 1465290, 1465712, +, 423, 0; cds-WP_001206197.1, NZ_CP025268.1, 1465712, 1466917, +, 1206, 0; cds-WP_000632280.1, NZ_CP025268.1, 1466944, 1468257, +, 1314, 0; cds-WP_000039887.1, NZ_CP025268.1, 1468358, 1469308, +, 951, 0; cds-WP_101348588.1, NZ_CP025268.1, 1469290, 1469880, +, 591, 0; cds-WP_010723099.1, NZ_CP025268.1, 1469984, 1470049, -, 66, 0; cds-CXP41 R507805, NZ_CP025268.1, 1470211, 1472736, +, 2526, 18; cds-WP_088895425.1-2, NZ CP025268.1;NZ CP025268.1, 1472740;1473653, 1473653;1473968, -;-, 1229, 12158; cds-WP_001254932.1, NZ_CP025268.1, 1474177, 1475328, +, 1152, 699; cds-CXP41 R507825, NZ_CP025268.1, 1475335, 1478832, +, 3498, 57179; cds-WP_00OO97801.1, NZ_CP025268.1, 1479040, 1479900, +, 861, 0; cds-WP_000910026.1, NZ_CP025268.1, 1479963, 1482269, +, 2307, 0; cds-WP_000177515.1, NZ _CP025268.1, 1482440, 1483045, +, 606, 0; cds-WP_000890941.1, NZ_CP025268.1, 1483045, 1483941, +, 897, 0; cds-WP_000431845.1, NZ_CP025268.1, 1483957, 1485714, +, 1758, 0; cds-WP_000627368.1, NZ_CP025268.1, 1485704, 1487020, +, 1317, 0; cds-WP_000048950.1, NZ_CP025268.1, 1487074, 1487679, -, 606, 59; cds-WP_000139543.1, NZ_CP025268.1, 1487880, 1491782, +, 3903, 127962; cds-WP_001027942.1, NZ_CP025268.1, 1492054, 1492854, +, 801, 0; cds-WP_000115943.1, NZ_CP025268.1, 1493051, 1494490, +, 1440, 16150; cds-CXP41_RS24180, NZ_CP025268.1, 1494532, 1495532, -, 1001, 18049; cds-WP_101348589.1, NZ_CP025268.1, 1495721, 1496251, +, 531, 66923; cds-WP_000731833.1, NZ_CP025268.1, 1496496, 1496669, +, 174, 12241; cds-WP_211180495.1, NZ_CP025268.1, 1496695, 1496763, +, 69, 646; cds-WP_001320773.1, NZ_CP025268.1, 1496741, 1496890, -, 150, 0; cds-WP_001098559.1, NZ_CP025268.1, 1497289, 1498929, +, 1641, 12972; cds-WP_000414567.1, NZ_CP025268.1, 1498967, 1499890, -, 924, 0; cds-WP_000013789.1, NZ_CP025268.1, 1500107, 1501450, +, 1344, 16; cds-WP_000375961.1, NZ_CP025268.1, 1501675, 1503330, +, 1656, 289281; cds-WP_001296778.1, NZ_CP025268.1, 1503470, 1503694, +, 225, 2; cds-WP_000140873.1, NZ_CP025268.1, 1503757, 1504296, +, 540, 0; cds-WP_001234042.1, NZ_CP025268.1, 1504288, 1505268, -, 981, 0; cds-WP_001301046.1, NZ_CP025268.1, 1505392, 1506384, +, 993, 51145; cds-WP_000586732.1, NZ_CP025268.1, 1506381, 1506974, +, 594, 306313; cds-WP_001261013.1, NZ_CP025268.1, 1507276, 1507944, +, 669, 241; cds-CXP41_RS07970, NZ_CP025268.1, 1507962, 1508468, -, 507, 0; cds-WP_000826416.1, NZ_CP025268.1, 1508476, 1509684, +, 1209, 4; cds-WP_001326689.1, NZ_CP025268.1, 1509724, 1510938, -, 1215, 5472; cds-WP_000429155.1, NZ_CP025268.1, 1510991, 1511527, +, 537, 0; cds-WP_001303492.1, NZ_CP025268.1, 1511600, 1513561, +, 1962, 2660; cds-WP _000494244.1, NZ_CP025268.1, 1513653, 1513883, -, 231, 3; cds-WP_000813794.1, NZ_CP025268.1, 1514105, 1514281, +, 177, 0; cds-WP_001270286.1, NZ_CP025268.1, 1514327, 1514743, +, 417, 0; cds-WP_000760626.1, NZ_CP025268.1, 1514822, 1516228, +, 1407, 375; cds-WP_000047424.1, NZ_CP025268.1, 1516473, 1517618, +, 1146, 0; cds-WP_101348590.1, NZ_CP025268.1, 1517636, 1518649, +, 1014, 0; cds-WP_001251304.1, NZ_CP025268.1, 1518650, 1519591, +, 942, 2; cds-WP_000555458.1, NZ_CP025268.1, 1519581, 1520375, +, 795, 0; cds-WP_001163872.1, NZ_CP025268.1, 1520397, 1521821, +, 1425, 0; cds-WP_001310798.1, NZ_CP025268.1, 1521918, 1522013, -, 96, 0; cds-WP_000061178.1, NZ_CP025268.1, 1522208, 1522381, +, 174, 6487; cds-WP_000018633.1, NZ_CP025268.1, 1522467, 1522700, +, 234, 8; cds-WP_001076535.1, NZ_CP025268.1, 1522701, 1523150, -, 450, 0; cds-WP_001310799.1, NZ_CP025268.1, 1523147, 1523665, -, 519, 0; cds-WP_000531462.1, NZ_CP025268.1, 1523846, 1524883, +, 1038, 0; cds-WP_001300742.1, NZ_CP025268.1, 1525081, 1525746, +, 666, 4983; cds-WP_000689363.1, NZ_CP025268.1, 1525782, 1527884, -, 2103, 315184; cds-WP_000550695.1, NZ_CP025268.1, 1528126, 1529187, +, 1062, 92409; cds-WP_001300590.1, NZ _CP025268.1, 1529300, 1530799, -, 1500, 373; cds-WP_000598857.1, NZ _CP025268.1, 1531066, 1531683, +, 618, 0; cds-WP_000226995.1, NZ_CP025268.1, 1531759, 1531971, +, 213, 0; cds-CXP41_RS08125, NZ_CP025268.1, 1532721, 1534757, +, 2037, 0; cds-WP_101348591.1, NZ_CP025268.1, 1534741, 1535223, +, 483, 2106; cds-WP_120795385.1, NZ_CP025268.1, 1535138, 1535323, -, 186, 4343; cds-CXP41_RS08135, NZ_CP025268.1, 1535405, 1536549, +, 1145, 14342; cds-WP_000421020.1, NZ_CP025268.1, 1536635, 1537771, +, 1137, 598; cds-WP_001120143.1, NZ_CP025268.1, 1537871, 1538104, +, 234, 0; cds-WP_000085899.1, NZ_CP025268.1, 1538101, 1538670, -, 570, 1; cds-WP_000187925.1, NZ_CP025268.1, 1538843, 1539688, +, 846, 5; cds-WP_000804385.1, NZ_CP025268.1, 1539784, 1540677, -, 894, 0; cds-WP_000617115.1, NZ_CP025268.1, 1540756, 1541436, -, 681, 0; cds-WP_001166353.1, NZ_CP025268.1, 1541433, 1542128, -, 696, 0; cds-WP_000702535.1, NZ_CP025268.1, 1542128, 1543672, -, 1545, 0; cds-WP_000040458.1, NZ_CP025268.1, 1543669, 1547409, -, 3741, 390; cds-WP_001207901.1, NZ_CP025268.1, 1547491, 1548879, -, 1389, 49; cds-CXP41_RS08190, NZ_CP025268.1, 1549203, 1550533, -, 1331, 0; cds-WP_000768384.1, NZ_CP025268.1, 1550557, 1550847, -, 291, 0; cds-WP_000198205.1, NZ_CP025268.1, 1551107, 1551988, -, 882, 351; cds-WP_157825723.1, NZ_CP025268.1, 1552220, 1555267, +, 3048, 3253; cds-WP_001240582.1, NZ_CP025268.1, 1555280, 1556164, +, 885, 1702; cds-WP_000045648.1, NZ_CP025268.1, 1556157, 1556810, +, 654, 818; cds-WP_000781370.1, NZ_CP025268.1, 1557217, 1557501, -, 285, 15; cds-WP_000642433.1, NZ_CP025268.1, 1557647, 1558657, -, 1011, 99; cds-WP_000433476.1, NZ_CP025268.1, 1558791, 1560488, -, 1698, 241640; cds-WP_000841554.1, NZ_CP025268.1, 1560645, 1560782, -, 138, 6; cds-WP_000495771.1, NZ_CP025268.1, 1560884, 1561099, -, 216, 0; cds-WP_000152305.1, NZ_CP025268.1, 1561444, 1561875, +, 432, 46254; cds-WP_001285536.1, NZ_CP025268.1, 1561931, 1562857, -, 927, 0; cds-WP_000193547.1, NZ_CP025268.1, 1562850, 1563836, -, 987, 0; cds-WP_000979626.1, NZ_CP025268.1, 1563833, 1564729, -, 897, 192; cds-WP_000145123.1, NZ_CP025268.1, 1564726, 1565748, -, 1023, 35; cds-WP_000830550.1, NZ_CP025268.1, 1565750, 1567300, -, 1551, 0; cds-WP_001285833.1, NZ_CP025268.1, 1567314, 1567895, -, 582, 0; cds-WP_001360132.1, NZ_CP025268.1, 1568153, 1570552, -, 2400, 133; cds-WP_000426292.1, NZ_CP025268.1, 1570577, 1571959, -, 1383, 0; cds-WP_120795386.1, NZ_CP025268.1, 1571997, 1572038, -, 42, 0; cds-WP_000350395.1, NZ_CP025268.1, 1572323, 1573642, -, 1320, 0; cds-WP_000246019.1, NZ_CP025268.1, 1573773, 1575308, -, 1536, 0; cds-WP_000358930.1, NZ_CP025268.1, 1575464, 1576864, -, 1401, 0; cds-WP_001244968.1, NZ_CP025268.1, 1577226, 1580009, -, 2784, 35309; cds-WP_000832437.1, NZ_CP025268.1, 1580066, 1582438, -, 2373, 0; cds-WP_000628576.1, NZ_CP025268.1, 1582476, 1584161, -, 1686, 0; cds-WP_120795387.1, NZ_CP025268.1, 1584364, 1584486, +, 123, 0; cds-WP_001301030.1, NZ_CP025268.1, 1584452, 1585609, -, 1158, 0; cds-WP_001295684.1, NZ_CP025268.1, 1585661, 1587343, -, 1683, 0; cds-WP_000060495.1, NZ_CP025268.1, 1587745, 1588506, -, 762, 0; cds-WP_000543384.1, NZ_CP025268.1, 1588581, 1588778, -, 198, 0; cds-WP 101348593.1, NZ_CP025268.1, 1589026, 1591305, -, 2280, 0; cds-WP_000520676.1, NZ_CP025268.1, 1591639, 1592553, -, 915, 0; cds-WP_000825452.1, NZ_CP025268.1, 1592612, 1593115, -, 504, 0; cds-WP_000876763.1, NZ_CP025268.1, 1593128, 1593658, -, 531, 0; cds-CXP41 R508370, NZ_CP025268.1, 1593672, 1594898, -, 1227, 2; cds-CXP41_RS08375, NZ_CP025268.1, 1595153, 1595361, -, 209, 0; cds-WP_001125439.1, NZ_CP025268.1, 1595673, 1596995, -, 1323, 168; cds-WP_001301023.1, NZ_CP025268.1, 1596995, 1597261, -, 267, 0; cds-CXP41_RS08400, NZ_CP025268.1, 1597484, 1602906, -, 5423, 2; cds-WP_000113108.1, NZ_CP025268.1, 1603437, 1605029, -, 1593, 14; cds-WP_000154342.1, NZ_CP025268.1, 1605108, 1606061, -, 954, 0; cds-WP_001194860.1, NZ_CP025268.1, 1606310, 1607845, +, 1536, 0; cds-WP_000911184.1, NZ_CP025268.1, 1607839, 1608867, +, 1029, 0; cds-WP_001222721.1, NZ_CP025268.1, 1608867, 1609859, +, 993, 0; cds-WP_000172465.1, NZ_CP025268.1, 1609871, 1610893, +, 1023, 0; cds-WP_000774165.1, NZ _CP025268.1, 1610920, 1611795, +, 876, 0; cds-WP_000558527.1, NZ_CP025268.1, 1611819, 1612109, +, 291, 0; cds-WP_001286597.1, NZ_CP025268.1, 1612166, 1612924, +, 759, 13311; cds-WP_001313828.1, NZ_CP025268.1, 1612928, 1613842, -, 915, 4; cds-WP_000854633.1, NZ_CP025268.1, 1614049, 1615500, -, 1452, 3; cds-CXP41 R508470, NZ_CP025268.1, 1615727, 1616713, -, 987, 2845; cds-WP_001191027.1, NZ_CP025268.1, 1616786, 1617145, -, 360, 158; cds-WP_000257409.1, NZ_CP025268.1, 1617145, 1618071, -, 927, 97025; cds-WP_000156615.1, NZ_CP025268.1, 1618135, 1619523, -, 1389, 70382; cds-WP_000366501.1, NZ_CP025268.1, 1619624, 1620505, +, 882, 39851; cds-WP_000258599.1, NZ_CP025268.1, 1620583, 1621698, +, 1116, 0; cds-WP_000210799.1, NZ_CP025268.1, 1621848, 1623038, +, 1191, 4; cds-WP_000885033.1, NZ_CP025268.1,
1623063, 1623728, -, 666, 0; cds-WP_000843414.1, NZ_CP025268.1, 1623940, 1624374, +, 435, 8963; cds-WP_000091199.1, NZ_CP025268.1, 1624394, 1624777, +, 384, 99081; cds-WP_000803657.1, NZ_CP025268.1, 1624809, 1625027, +, 219, 184273; cds-WP_000087187.1, NZ_CP025268.1, 1625058, 1625957, -, 900, 20860; cds-WP_101348595.1, NZ_CP025268.1, 1626152, 1627339, +, 1188, 0; cds-WP_000901367.1, NZ_CP025268.1, 1627466, 1627561, +, 96, 1658; cds-WP_211180496.1, NZ_CP025268.1, 1627561, 1627665, +, 105, 137; cds-WP_000592814.1, NZ_CP025268.1, 1627780, 1628670, -, 891, 11289; cds-CXP41_R508545, NZ_CP025268.1, 1628925, 1629316, -, 392, 0; cds-WP_001024746.1, NZ_CP025268.1, 1629592, 1630110, +, 519, 0; cds-WP_101348596.1, NZ_CP025268.1, 1630154, 1632199, -, 2046, 4167; cds-WP_000636571.1, NZ_CP025268.1, 1632336, 1633082, +, 747, 144; cds-WP_000215549.1, NZ_CP025268.1, 1633171, 1633857, +, 687, 70174; cds-WP_000214712.1, NZ_CP025268.1, 1634034, 1634237, +, 204, 88183; cds-WP_000527743.1, NZ_CP025268.1, 1634272, 1635732, -, 1461, 3; cds-CXP41_RS08580, NZ_CP025268.1, 1635821, 1637188, -, 1368, 0; cds-WP_120795384.1-2, NZ_CP025268.1, 1637709, 1637822, +, 114, 5664; cds-WP_000836768.1, NZ_CP025268.1, 1637891, 1638124, +, 234, 20370; cds-WP_000078177.1, NZ_CP025268.1, 1638441, 1639031, +, 591, 0; cds-WP_000885611.1, NZ_CP025268.1, 1639129, 1639704, -, 576, 0; cds-WP_001027733.1, NZ_CP025268.1, 1639704, 1640666, -, 963, 0; cds-WP_000453612.1, NZ_CP025268.1, 1640617, 1641186, -, 570, 0; cds-WP_001368374.1, NZ_CP025268.1, 1641575, 1641808, +, 234, 0; cds-WP_000373090.1, NZ_CP025268.1, 1641866, 1642276, +, 411, 0; cds-WP_001019606.1, NZ_CP025268.1, 1642428, 1642601, -, 174, 3584; cds-WP_001309517.1, NZ_CP025268.1, 1642773, 1642928, -, 156, 2; cds-WP_120795388.1, NZ_CP025268.1, 1643007, 1643072, -, 66, 0; cds-WP_071524604.1, NZ_CP025268.1, 1643075, 1643263, -, 189, 117; cds-WP_000066495.1, NZ_CP025268.1, 1643274, 1643486, -, 213, 113; cds-WP_001071769.1, NZ_CP025268.1, 1643849, 1644346, -, 498, 0; cds-WP_001092971.1, NZ_CP025268.1, 1644343, 1644876, -, 534, 0; cds-WP_000189916.1, NZ_CP025268.1, 1644873, 1645184, -, 312, 941; cds-WP_000839590.1, NZ_CP025268.1, 1645189, 1645404, -, 216, 0; cds-WP_211180497.1, NZ_CP025268.1, 1645624, 1645794, +, 171, 0; cds-WP_000066484.1, NZ_CP025268.1, 1646158, 1646373, -, 216, 92777; cds-WP_000087756.1, NZ_CP025268.1, 1646674, 1646886, +, 213, 85166; cds-WP_120795389.1, NZ_CP025268.1, 1646941, 1647030, +, 90, 122089; cds-WP_001047135.1, NZ_CP025268.1, 1647308, 1648060, -, 753, 120657; cds-WP_001393597.1, NZ_CP025268.1, 1648074, 1649123, -, 1050, 0; cds-WP_012304870.1, NZ_CP025268.1, 1649125, 1649403, -, 279, 29; cds-WP_000980994.1, NZ_CP025268.1, 1649470, 1649721, -, 252, 0; cds-WP_000813254.1, NZ_CP025268.1, 1649938, 1650093, -, 156, 0; cds-WP_000323025.1, NZ_CP025268.1, 1650165, 1650452, -, 288, 4; cds-WP_000534858.1, NZ_CP025268.1, 1650452, 1650691, -, 240, 44; cds-WP_001326990.1, NZ_CP025268.1, 1650716, 1651021, +, 306, 0; cds-WP_001301033.1, NZ_CP025268.1, 1651224, 1651556, +, 333, 6; cds-WP_011443592.1, NZ_CP025268.1, 1651993, 1652142, -, 150, 0; cds-CXP41_RS08725, NZ_CP025268.1, 1652177, 1652455, -, 279, 0; cds-WP_000920568.1, NZ_CP025268.1, 1652439, 1652669, -, 231, 0; cds-WP_000448564.1, NZ_CP025268.1, 1652753, 1653160, +, 408, 931; cds-WP_000379575.1, NZ_CP025268.1, 1653327, 1653482, +, 156, 0; cds-WP_001171942.1, NZ_CP025268.1, 1653642, 1653860, +, 219, 0; cds-WP_000854559.1, NZ_CP025268.1, 1654428, 1654616, +, 189, 0; cds-WP_001083297.1, NZ_CP025268.1, 1654613, 1654804, +, 192, 0; cds-CXP41_R508770, NZ_CP025268.1, 1654897, 1655664, +, 768, 0; cds-CXP41_RS08775, NZ_CP025268.1, 1655661, 1656356, +, 696, 8; cds-CXP41_RS08780, NZ_CP025268.1, 1656370, 1657527, +, 1158, 0; cds-WP_000836066.1, NZ_CP025268.1, 1657715, 1658734, -, 1020, 0; cds-WP_101348598.1, NZ_CP025268.1, 1658746, 1659960, -, 1215, 0; cds-WP_101348599.1, NZ_CP025268.1, 1660141, 1660491, -, 351, 0; cds-WP_000705211.1, NZ_CP025268.1, 1660626, 1660967, +, 342, 15355; cds-WP_001138581.1, NZ_CP025268.1, 1661002, 1661562, +, 561, 309704; cds-WP_001321287.1, NZ_CP025268.1, 1661565, 1662275, -, 711, 2; cds-WP_001300836.1, NZ_CP025268.1, 1662383, 1662688, +, 306, 751; cds-WP_000041675.1, NZ_CP025268.1, 1662887, 1665313, +, 2427, 1; cds-WP_001340362.1, NZ_CP025268.1, 1665374, 1667797, +, 2424, 105; cds-WP_000213028.1, NZ_CP025268.1, 1667808, 1668425, +, 618, 70932; cds-WP_101348600.1, NZ_CP025268.1, 1668427, 1669281, +, 855, 1; cds-WP_000148710.1, NZ_CP025268.1, 1669324, 1669938, +, 615, 3; cds-WP_071592181.1, NZ_CP025268.1, 1670097, 1671389, +, 1293, 222; cds-WP_000919231.1, NZ_CP025268.1, 1671342, 1672037, -, 696, 0; cds-WP_000225262.1, NZ_CP025268.1, 1672162, 1673382, -, 1221, 22924; cds-WP_001019525.1, NZ_CP025268.1, 1673517, 1674410, -, 894, 0; cds-WP_101348601.1, NZ_CP025268.1, 1674517, 1675770, +, 1254, 5532; cds-WP_001299090.1, NZ_CP025268.1, 1675819, 1675938, -, 120, 0; cds-WP_001340364.1, NZ_CP025268.1, 1676194, 1676502, +, 309, 0; cds-WP_000233093.1, NZ_CP025268.1, 1676595, 1676678, +, 84, 0; cds-WP_001260865.1, NZ_CP025268.1, 1676778, 1677599, +, 822, 0; cds-WP_000046661.1, NZ_CP025268.1, 1677638, 1677967, -, 330, 0; cds-CXP41_RS08905, NZ_CP025268.1, 1677954, 1678320, -, 367, 12800; cds-WP_211180498.1, NZ_CP025268.1, 1678427, 1678528, -, 102, 3399; cds-WP_001118241.1, NZ_CP025268.1, 1678732, 1679766, +, 1035, 250; cds-WP_000014036.1, NZ_CP025268.1, 1679791, 1681179, -, 1389, 233571; cds-WP_001300486.1, NZ_CP025268.1, 1681190, 1682722, -, 1533, 160606; cds-WP_000769303.1, NZ_CP025268.1, 1683246, 1684190, +, 945, 151865; cds-WP_000412379.1, NZ_CP025268.1, 1684376, 1685758, +, 1383, 100133; cds-WP_000520804.1, NZ_CP025268.1, 1685795, 1686517, +, 723, 5350; cds-WP_000524868.1, NZ_CP025268.1, 1686514, 1686849, -, 336, 559; cds-WP_001092508.1, NZ_CP025268.1, 1686978, 1687697, +, 720, 1115; cds-WP_000732512.1, NZ_CP025268.1, 1687701, 1689002, +, 1302, 115904; cds-WP_000135181.1, NZ_CP025268.1, 1689078, 1690007, +, 930, 45; cds-CXP41_RS08970, NZ_CP025268.1, 1690004, 1691406, -, 1403, 295; cds-WP_000066639.1, NZ_CP025268.1, 1691549, 1693195, -, 1647, 33; cds-WP_001170664.1, NZ_CP025268.1, 1693394, 1694569, +, 1176, 23829; cds-WP_001043339.1, NZ_CP025268.1, 1694670, 1696178, +, 1509, 83554; cds-WP_001227023.1, NZ_CP025268.1, 1696404, 1697669, -, 1266, 0; cds-WP_001075849.1, NZ_CP025268.1, 1697708, 1699081, -, 1374, 0; cds-WP_000945878.1, NZ_CP025268.1, 1699078, 1700889, -, 1812, 3; cds-WP_000969092.1, NZ_CP025268.1, 1701279, 1701869, -, 591, 1895; cds-WP_000483353.1, NZ_CP025268.1, 1702090, 1702857, -, 768, 1102; cds-WP_000179510.1, NZ_CP025268.1, 1702969, 1703997, -, 1029, 0; cds-CXP41_RS09020, NZ_CP025268.1, 1704172, 1705765, +, 1594, 1; cds-WP_000459379.1, NZ_CP025268.1, 1705775, 1706947, +, 1173, 4; cds-WP_000567490.1, NZ_CP025268.1, 1707051, 1708052, +, 1002, 14885; cds-WP_001282550.1, NZ_CP025268.1, 1708086, 1709126, -, 1041, 135653; cds-WP_001300888.1, NZ_CP025268.1, 1709369, 1709494, +, 126, 0; cds-WP_000217950.1, NZ_CP025268.1, 1709767, 1709982, +, 216, 0; cds-WP_001310854.1, NZ_CP025268.1, 1710068, 1710508, +, 441, 381; cds-WP_000133193.1, NZ_CP025268.1, 1710585, 1711166, +, 582, 9349; cds-WP_000991805.1, NZ_CP025268.1, 1711166, 1711744, +, 579, 79106; cds-WP_000915778.1, NZ_CP025268.1, 1711737, 1713959, +, 2223, 613504; cds-WP_101348602.1, NZ_CP025268.1, 1713960, 1715018, +, 1059, 466319; cds-WP_000920784.1, NZ_CP025268.1, 1715022, 1715642, +, 621, 84542; cds-WP_001289652.1, NZ_CP025268.1, 1715646, 1716341, +, 696, 111854; cds-WP_001030339.1, NZ_CP025268.1, 1716341, 1716976, +, 636, 34696; cds-WP_000100932.1, NZ_CP025268.1, 1717588, 1719090, +, 1503, 930937; cds-WP_000765749.1, NZ_CP025268.1, 1719196, 1719801, +, 606, 47425; cds-WP_001307229.1, NZ_CP025268.1, 1719845, 1720708, -, 864, 72; cds-WP_001295400.1, NZ_CP025268.1, 1720767, 1722041, -, 1275, 233030; cds-WP_001282319.1, NZ_CP025268.1, 1722170, 1722826, -, 657, 5558; cds-WP_000178044.1, NZ_CP025268.1, 1722885, 1723214, -, 330, 324; cds-WP_101348603.1, NZ_CP025268.1, 1723312, 1724421, -, 1110, 2; cds-WP_000597196.1, NZ_CP025268.1, 1724695, 1725162, +, 468, 36643; cds-WP_001296943.1, NZ_CP025268.1, 1725209, 1725643, -, 435, 78869; cds-WP_000670998.1, NZ_CP025268.1, 1725844, 1726080, +, 237, 117613; cds-WP_001300356.1, NZ_CP025268.1, 1726083, 1726940, +, 858, 18034; cds-WP_000994333.1, NZ_CP025268.1, 1726940, 1728952, +, 2013, 11899; cds-WP_001296937.1, NZ_CP025268.1, 1728953, 1729474, -, 522, 113543; cds-WP_000250656.1, NZ_CP025268.1, 1729555, 1730451, -, 897, 11278; cds-WP_000840481.1, NZ_CP025268.1, 1730500, 1730739, -, 240, 0; cds-WP_001032936.1, NZ_CP025268.1, 1730842, 1731441, +, 600, 273874; cds-WP_000093589.1, NZ_CP025268.1, 1731478, 1732575, +, 1098, 3141; cds-WP_001237796.1, NZ_CP025268.1, 1732656, 1733063, +, 408, 46197; cds-WP_001282283.1, NZ_CP025268.1, 1733166, 1733813, +, 648, 248; cds-WP_001310858.1, NZ_CP025268.1, 1733906, 1738522, +, 4617, 307; cds-WP_000108172.1, NZ_CP025268.1, 1738573, 1738920, -, 348, 278; cds-WP_000101193.1, NZ_CP025268.1, 1739254, 1740069, +, 816, 31; cds-WP_101348604.1, NZ_CP025268.1, 1740197, 1740778, +, 582, 383332; cds-WP_000701040.1, NZ_CP025268.1, 1740940, 1742109, -, 1170, 16; cds-WP_000102278.1, NZ_CP025268.1, 1742275, 1742364, -, 90, 0; cds-WP_000190982.1, NZ_CP025268.1, 1742663, 1743688, +, 1026, 127800; cds-WP_000269501.1, NZ_CP025268.1, 1743685, 1744617, -, 933, 893; cds-WP_001182347.1, NZ_CP025268.1, 1744730, 1745941, +, 1212, 29983; cds-WP_000098896.1, NZ_CP025268.1, 1746232, 1747380, +, 1149, 199430; cds-WP_000493947.1, NZ_CP025268.1, 1747420, 1748061, -, 642, 0; cds-WP_001174940.1, NZ_CP025268.1, 1748276, 1749649, +, 1374, 17667; cds-WP_000534271.1, NZ_CP025268.1, 1749690, 1750946, -, 1257, 243803; cds-WP_000212657.1, NZ_CP025268.1, 1751519, 1751824, +, 306, 78478; cds-WP_000716950.1, NZ_CP025268.1, 1751950, 1753554, +, 1605, 62498; cds-WP_000587525.1, NZ_CP025268.1, 1753566, 1754378, -, 813, 0; cds-WP_001069979.1, NZ_CP025268.1, 1754382, 1755167, -, 786, 330; cds-WP_001310861.1, NZ_CP025268.1, 1755164, 1755832, -, 669, 0; cds-WP_001301419.1, NZ_CP025268.1, 1755896, 1756534, -, 639, 0; cds-WP_001273527.1, NZ_CP025268.1, 1756547, 1758649, -, 2103, 0; cds-WP_001070230.1, NZ_CP025268.1, 1758670, 1759296, -, 627, 14; cds-WP_000528342.1, NZ_CP025268.1, 1759751, 1759960, -, 210, 0; cds-WP_101348605.1, NZ_CP025268.1, 1760517, 1761929, +, 1413, 1554104; cds-WP_000648420.1, NZ_CP025268.1, 1762240, 1762476, +, 237, 3406; cds-CXP41_RS09330, NZ_CP025268.1, 1762540, 1763539, -, 1000, 12314; cds-WP_001196530.1, NZ_CP025268.1, 1763688, 1764104, -, 417, 2; cds-CXP41_RS09340, NZ_CP025268.1, 1764117, 1765338, -, 1222, 306087; cds-WP_101348606.1, NZ_CP025268.1, 1765335, 1766606, -, 1272, 0; cds-WP_000948863.1, NZ_CP025268.1, 1766581, 1767327, -, 747, 2048; cds-CXP41_RS09355, NZ_CP025268.1, 1767337, 1768823, -, 1487, 0; cds-WP_000367160.1, NZ_CP025268.1, 1768832, 1769200, -, 369, 6; cds-WP_001296104.1, NZ_CP025268.1, 1769748, 1769936, -, 189, 0; cds-WP_000637982.1, NZ_CP025268.1, 1770036, 1770446, -, 411, 1993; cds-WP_000613008.1, NZ_CP025268.1, 1770443, 1773499, -, 3057, 43930; cds-WP_101348607.1, NZ_CP025268.1, 1773888, 1775003, +, 1116, 98; cds-WP_001310864.1, NZ_CP025268.1, 1775432, 1775788, +, 357, 0; cds-WP_000795537.1, NZ_CP025268.1, 1775888, 1777102, +, 1215, 0; cds-WP_000081197.1, NZ_CP025268.1, 1777329, 1778594, +, 1266, 0; cds-WP_000383469.1, NZ_CP025268.1, 1778606, 1779472, +, 867, 0; cds-WP_000860201.1, NZ_CP025268.1, 1779503, 1780261, +, 759, 0; cds-WP_000805700.1, NZ_CP025268.1, 1780404, 1781999, +, 1596, 0; cds-WP_000347850.1, NZ_CP025268.1, 1782013, 1783164, +, 1152, 0; cds-WP_000284799.1, NZ_CP025268.1, 1783207, 1784118, -, 912, 0; cds-WP_000692135.1, NZ_CP025268.1, 1784434, 1785198, +, 765, 0; cds-WP_000080722.1, NZ_CP025268.1, 1785218, 1786156, +, 939, 1; cds-WP_001278525.1, NZ_CP025268.1, 1786212, 1787501, +, 1290, 127; cds-WP_000081081.1, NZ_CP025268.1, 1787498, 1787791, +, 294, 0; cds-WP_001336282.1, NZ_CP025268.1, 1787848, 1789494, +, 1647, 0; cds-WP_000069375.1, NZ_CP025268.1, 1789551, 1791929, -, 2379, 15; cds-WP_000368046.1, NZ_CP025268.1, 1792262, 1793095, +, 834, 0; cds-WP_001082229.1, NZ_CP025268.1, 1793252, 1794298, +, 1047, 1819; cds-WP_001270809.1, NZ_CP025268.1, 1794430, 1794621, +, 192, 42; cds-WP_000175703.1, NZ_CP025268.1, 1794625, 1796061, -, 1437, 29900; cds-
WP_001300634.1, NZ_CP025268.1, 1796124, 1796837, -, 714, 73568; cds-WP _001209795.1, NZ_CP025268.1, 1797084, 1797548, -, 465, 1671; cds-WP _000029474.1, NZ_CP025268.1, 1797626, 1798375, -, 750, 1; cds-WP_001154168.1, NZ_CP025268.1, 1798375, 1798926, -, 552, 0; cds-WP_000956528.1, NZ_CP025268.1, 1798989, 1799969, -, 981, 1374; cds-WP _001229265.1, NZ_CP025268.1, 1800070, 1800369, -, 300, 11548; cds-WP _000672380.1, NZ_CP025268.1, 1800374, 1802761, -, 2388, 2320260; cds-WP_000018596.1, NZ_CP025268.1, 1802776, 1803759, -, 984, 652332; cds-WP_001386830.1, NZ_CP025268.1, 1804043, 1804087, -, 45, 0; cds-WP_000124850.1, NZ_CP025268.1, 1804210, 1804566, -, 357, 2136197; cds-WP _001124225.1, NZ_CP025268.1, 1804619, 1804816, -, 198, 1505527; cds-WP_001700733.1, NZ_CP025268.1, 1804913, 1805455, -, 543, 1388071; cds-WP _001144202.1, NZ_CP025268.1, 1805459, 1807387, -, 1929, 2954188; cds-CXP41_RS09565, NZ_CP025268.1, 1807911, 1809810, +, 1900, 510; cds-WP_001142445.1, NZ_CP025268.1, 1809982, 1810089, +, 108, 0; cds-WP_000771391.1, NZ_CP025268.1, 1810142, 1810900, -, 759, 5; cds-WP _000251727.1, NZ_CP025268.1, 1811187, 1812116, +, 930, 15; cds-WP _000146159.1, NZ_CP025268.1, 1812217, 1812507, +, 291, 0; cds-WP_000267650.1, NZ_CP025268.1, 1812613, 1813473, +, 861, 0; cds-WP_000222163.1, NZ_CP025268.1, 1813514, 1814050, -, 537, 10112; cds-WP_000106833.1, NZ_CP025268.1, 1814197, 1814865, +, 669, 1779; cds-WP _001297653.1, NZ_CP025268.1, 1815028, 1815618, +, 591, 6; cds-WP _001010696.1, NZ_CP025268.1, 1815751, 1817142, +, 1392, 1356; cds-WP_001310874.1, NZ_CP025268.1, 1817146, 1817949, -, 804, 12125; cds-WP_001241561.1, NZ_CP025268.1, 1818238, 1818501, -, 264, 0; cds-WP _000077872.1, NZ_CP025268.1, 1818684, 1820945, +, 2262, 2792; cds-WP_000440471.1, NZ_CP025268.1, 1821203, 1821952, -, 750, 0; cds-WP_000078765.1, NZ_CP025268.1, 1821965, 1823317, -, 1353, 0; cds-WP_000983647.1, NZ_CP025268.1, 1823422, 1824264, -, 843, 0; cds-WP _000968919.1, NZ_CP025268.1, 1824272, 1824622, -, 351, 0; cds-WP_000073041.1, NZ_CP025268.1, 1824673, 1826031, -, 1359, 0; cds-WP_000412169.1, NZ_CP025268.1, 1826116, 1826436, -, 321, 24; cds-WP_001039044.1, NZ_CP025268.1, 1826734, 1827072, -, 339, 0; cds-WP_000175026.1, NZ_CP025268.1, 1827274,1828101, +, 828, 7026; cds-WP _000252397.1, NZ_CP025268.1, 1828331, 1829218, +, 888, 251; cds-WP _001300480.1, NZ_CP025268.1, 1829178, 1829753, -, 576, 0; cds-WP_001228984.1, NZ_CP025268.1, 1829956, 1830441, -, 486, 0; cds-WP _000368506.1, NZ_CP025268.1, 1830771, 1831739, -, 969, 0; cds-WP_000994973.1, NZ_CP025268.1, 1831732, 1833075, -, 1344, 0; cds-WP _000177206.1, NZ_CP025268.1, 1833072, 1834550, -, 1479, 14; cds-WP_000989419.1, NZ_CP025268.1, 1834547, 1835581, -, 1035, 0; cds-WP_000081983.1, NZ_CP025268.1, 1835578, 1836798, -, 1221, 0; cds-WP _000673918.1, NZ_CP025268.1, 1837244, 1838050, +, 807, 297444; cds-WP _001300395.1, NZ_CP025268.1, 1838217, 1838927, +, 711, 16101; cds-WP _000939212.1, NZ_CP025268.1, 1838932, 1839609, +, 678, 0; cds-WP_000980098.1, NZ_CP025268.1, 1839624, 1840331, +, 708, 0; cds-WP _000524097.1, NZ_CP025268.1, 1840331, 1840879, +, 549, 0; cds-WP _001215339.1, NZ_CP025268.1, 1840889, 1842055, +, 1167, 0; cds-WP_000224958.1, NZ_CP025268.1, 1842028, 1843563, +, 1536, 0; cds-WP_001300558.1, NZ_CP025268.1, 1843563, 1844216, +, 654, 9; cds-WP _001350515.1, NZ_CP025268.1, 1844283, 1845590, +, 1308, 121; cds-WP _001295484.1, NZ_CP025268.1, 1845599, 1846219, -, 621, 54; cds-WP _000781888.1, NZ_CP025268.1, 1846306, 1846713, +, 408, 0; cds-WP_101348608.1, NZ_CP025268.1, 1846679, 1846951, -, 273, 0; cds-WP _000373021.1, NZ_CP025268.1, 1847187, 1848530, +, 1344, 0; cds-WP _000756910.1, NZ_CP025268.1, 1848647, 1849687, -, 1041, 20241; cds-WP_001235800.1, NZ_CP025268.1, 1849815, 1851776, -, 1962, 276660; cds-WP _001295485.1, NZ_CP025268.1, 1851781, 1852824, -, 1044, 803479; cds-WP _000339283.1, NZ_CP025268.1, 1852941, 1853492, -, 552, 28944; cds-WP _001259810.1, NZ_CP025268.1, 1853653, 1855509, +, 1857, 30; cds-WP_001170162.1, NZ_CP025268.1, 1855675, 1856691, +, 1017, 105360; cds-WP_001135066.1, NZ_CP025268.1, 1856702, 1857343, +, 642, 6; cds-WP _000438807.1, NZ_CP025268.1, 1857436, 1858794, -, 1359, 1000; cds-WP_000719096.1, NZ_CP025268.1, 1858911, 1859669, -, 759, 0; cds-WP _000723710.1, NZ_CP025268.1, 1859806, 1860786, -, 981, 0; cds-WP _000369231.1, NZ_CP025268.1, 1860796, 1861743, -, 948, 589; cds-WP _000878893.1, NZ_CP025268.1, 1861748, 1862584, -, 837, 0; cds-WP_000798110.1, NZ_CP025268.1, 1862605, 1863648, -, 1044, 0; cds-WP _000435292.1, NZ_CP025268.1, 1863665, 1865044, -, 1380, 0; cds-WP_000645221.1, NZ_CP025268.1, 1865071, 1866147, -, 1077, 0; cds-WP _001300415.1, NZ _CP025268.1, 1866517, 1866789, -, 273, 15142; cds-WP _001284618.1, NZ_CP025268.1, 1866831, 1867244, -, 414, 114524; cds-WP_000153502.1, NZ_CP025268.1, 1867586, 1868581, +, 996, 1396973; cds-WP _001335909.1, NZ_CP025268.1, 1868665, 1869549, +, 885, 3646; cds-WP _001186371.1, NZ_CP025268.1, 1869597, 1870451, -, 855, 5318; cds-WP _000163771.1, NZ_CP025268.1, 1870541, 1871287, -, 747, 234387; cds-WP_001019882.1, NZ_CP025268.1, 1871723, 1873657, +, 1935, 8973; cds-CXP41_RS09905, NZ_CP025268.1, 1873770, 1875052, +, 1283, 0; cds-WP _000616433.1, NZ_CP025268.1, 1875199, 1876674, +, 1476, 0; cds-WP_000766132.1, NZ_CP025268.1, 1876855, 1878345, +, 1491, 7; cds-WP_000138043.1, NZ_CP025268.1, 1878388, 1878891, +, 504, 0; cds-WP _000999630.1, NZ_CP025268.1, 1878892, 1878996, -, 105, 0; cds-WP _000460707.1, NZ_CP025268.1, 1879166, 1879612, +, 447, 0; cds-CXP41_RS09935, NZ_CP025268.1, 1879569, 1880389, -, 821, 4955; cds-WP_000128491.1, NZ_CP025268.1, 1880486, 1881667, +, 1182, 2; cds-WP_001326526.1, NZ_CP025268.1, 1881722, 1882069, +, 348, 2; cds-WP _000691930.1, NZ_CP025268.1, 1882091, 1882345, -, 255, 0; cds-WP _001310896.1, NZ_CP025268.1, 1882528, 1883553, +, 1026, 7; cds-WP_001219350.1, NZ_CP025268.1, 1883586, 1883684, +, 99, 2; cds-WP_001386836.1, NZ_CP025268.1, 1883687, 1883761, +, 75, 0; cds-WP_000512153.1, NZ_CP025268.1, 1883820, 1884068, -, 249, 0; cds-WP _000513737.1, NZ_CP025268.1, 1884216, 1884398, -, 183, 0; cds-WP _000939317.1, NZ_CP025268.1, 1884402, 1884761, -, 360, 90; cds-WP _000457206.1, NZ_CP025268.1, 1884934, 1885572, -, 639, 3147; cds-WP_101348722.1, NZ_CP025268.1, 1885699, 1886622, -, 924, 0; cds-WP _000978494.1, NZ_CP025268.1, 1886725, 1887810, +, 1086, 0; cds-CXP41_RS10000, NZ_CP025268.1, 1887965, 1889446, +, 1482, 0; cds-WP_000067822.1, NZ_CP025268.1, 1889478, 1890602, +, 1125, 0; cds-WP_001287026.1, NZ_CP025268.1, 1890658, 1891623, +, 966, 35471; cds-WP_001307844.1, NZ_CP025268.1, 1891677, 1892792, -, 1116, 18232; cds-WP_000758422.1, NZ_CP025268.1, 1892874, 1894559, -, 1686, 82298; cds-WP _000290576.1, NZ_CP025268.1, 1894764, 1895345, -, 582, 53331; cds-WP_001220966.1, NZ_CP025268.1, 1895385, 1896080, -, 696, 41924; cds-WP_000128841.1, NZ_CP025268.1, 1896138, 1898048, -, 1911, 630259; cds-WP _001295493.1, NZ_CP025268.1, 1898180, 1898524, +, 345, 669; cds-WP _001111995.1, NZ_CP025268.1, 1898946, 1899245, +, 300, 0; cds-WP_000457334.1, NZ_CP025268.1, 1899365, 1899544, -, 180, 0; cds-WP_000854958.1, NZ_CP025268.1, 1899618, 1900979, +, 1362, 27; cds-WP_000456715.1, NZ_CP025268.1, 1900983, 1901561, +, 579, 163; cds-WP _000624298.1, NZ_CP025268.1, 1901745, 1903109, +, 1365, 198212; cds-WP_001295494.1, NZ_CP025268.1, 1903240, 1904838, +, 1599, 0; cds-WP _000394983.1, NZ_CP025268.1, 1904842, 1906398, -, 1557, 1; cds-WP _000150543.1, NZ_CP025268.1, 1906861, 1907832, +, 972, 495; cds-WP _000406926.1, NZ_CP025268.1, 1907895, 1908695, +, 801, 68622; cds-WP_000228655.1, NZ_CP025268.1, 1908708, 1909559, +, 852, 1141; cds-WP _000156255.1, NZ_CP025268.1, 1909614, 1910072, +, 459, 3; cds-WP _021542535.1, NZ_CP025268.1, 1910501, 1911067, +, 567, 3087; cds-WP _000010115.1, NZ_CP025268.1, 1911064, 1911873, -, 810, 55; cds-WP_001062678.1, NZ_CP025268.1, 1912039, 1912248, -, 210, 3786; cds-WP_001295496.1, NZ_CP025268.1, 1912261, 1912404, -, 144, 35455; cds-WP _001006866.1, NZ_CP025268.1, 1913074, 1913361, -, 288, 5158; cds-WP _000714550.1, NZ_CP025268.1, 1913436, 1913579, -, 144, 61; cds-WP _001211011.1, NZ_CP025268.1, 1913738, 1913977, +, 240, 4; cds-WP _001262203.1, NZ_CP025268.1, 1914121, 1914912, -, 792, 150427; cds-WP_101348609.1, NZ_CP025268.1, 1915089, 1916462, +, 1374, 5; cds-WP_000984520.1, NZ_CP025268.1, 1916508, 1917389, -, 882, 166532; cds-WP_001055791.1, NZ_CP025268.1, 1917581, 1919629, -, 2049, 682104; cds-WP _000431381.1, NZ_CP025268.1, 1919649, 1920347, -, 699, 518391; cds-WP _001043877.1, NZ_CP025268.1, 1920444, 1920941, -, 498, 98200; cds-WP_001207282.1, NZ_CP025268.1, 1921071, 1922354, +, 1284, 6; cds-WP _001326728.1, NZ_CP025268.1, 1922323, 1924956, +, 2634, 31807; cds-WP_001352260.1, NZ_CP025268.1, 1925037, 1926476, +, 1440, 54423; cds-WP_001295499.1, NZ_CP025268.1, 1926594, 1926830, +, 237, 5615; cds-WP _000457842.1, NZ_CP025268.1, 1926935, 1927126, +, 192, 209; cds-WP _000812724.1, NZ _CP025268.1, 1927127, 1927783, -, 657, 6; cds-WP _000976476.1, NZ_CP025268.1, 1928179, 1928520, -, 342, 3075; cds-WP_000879289.1, NZ_CP025268.1, 1928533, 1929405, -, 873, 8023; cds-WP_000168747.1, NZ_CP025268.1, 1929409, 1929783, -, 375, 6524; cds-WP _000916763.1, NZ_CP025268.1, 1929922, 1930152, +, 231, 345; cds-WP_000011658.1, NZ_CP025268.1, 1930254, 1930910, +, 657, 1; cds-WP _000944256.1, NZ_CP025268.1, 1930934, 1931596, +, 663, 84; cds-WP _000936927.1, NZ_CP025268.1, 1931593, 1933653, -, 2061, 425; cds-WP_000024757.1, NZ_CP025268.1, 1933862, 1934521, -, 660, 427; cds-WP_001350516.1, NZ_CP025268.1, 1934848, 1935204, -, 357, 1093; cds-WP_000257738.1, NZ_CP025268.1, 1935271, 1935561, -, 291, 944; cds-CXP41_RS10265, NZ_CP025268.1, 1935695, 1936874, +, 1180, 0; cds-WP _000800512.1, NZ_CP025268.1, 1936930, 1937571, -, 642, 44831; cds-WP _001069467.1, NZ_CP025268.1, 1937608, 1939419, -, 1812, 73068; cds-WP_000301727.1, NZ_CP025268.1, 1939654, 1941129, -, 1476, 359265; cds-WP _001056706.1, NZ_CP025268.1, 1941467, 1942336, +, 870, 11133; cds-WP_000091148.1, NZ_CP025268.1, 1942464, 1943906, +, 1443, 5242; cds-WP_101348610.1, NZ_CP025268.1, 1944037, 1945008, -, 972, 26215; cds-WP_001184045.1, NZ_CP025268.1, 1945128, 1946450, -, 1323, 179727; cds-WP _001300644.1, NZ_CP025268.1, 1946466, 1947398, -, 933, 151142; cds-WP_101348611.1, NZ_CP025268.1, 1947477, 1948232, +, 756, 25733; cds-WP _000571480.1, NZ_CP025268.1, 1948229, 1949014, +, 786, 81; cds-WP _000568519.1, NZ_CP025268.1, 1949161, 1950171, -, 1011, 1552; cds-WP _000580323.1, NZ_CP025268.1, 1950180, 1950791, -, 612, 3; cds-WP_010723105.1, NZ_CP025268.1, 1950930, 1950995, -, 66, 0; cds-WP_001024932.1, NZ_CP025268.1, 1951066, 1951668, +, 603, 64548; cds-WP_001295503.1, NZ_CP025268.1, 1951670, 1952191, -, 522, 62958; cds-WP_000907248.1, NZ_CP025268.1, 1952226, 1952966, -, 741, 302189; cds-WP_001300367.1, NZ_CP025268.1, 1952995, 1953447, -, 453, 158832; cds-WP _001258678.1, NZ_CP025268.1, 1953565, 1955337, -, 1773, 559912; cds-WP _000891621.1, NZ_CP025268.1, 1955648, 1956214, +, 567, 149; cds-WP_000639274.1, NZ_CP025268.1, 1956211, 1957029, +, 819, 295; cds-WP_000252980.1, NZ_CP025268.1, 1957082, 1957477, +, 396, 1388; cds-WP _000019590.1, NZ_CP025268.1, 1957518, 1958261, +, 744, 88607; cds-WP_000564725.1, NZ_CP025268.1, 1958258, 1959229, +, 972, 44494; cds-WP_000176781.1, NZ_CP025268.1, 1959394, 1961823, -, 2430, 0; cds-WP _001214331.1, NZ_CP025268.1, 1961848, 1962948, -, 1101, 8; cds-WP _001185741.1, NZ_CP025268.1, 1963336, 1964082, -, 747, 305; cds-WP_001326725.1, NZ_CP025268.1, 1964096, 1964662, -, 567, 5; cds-WP_001025322.1, NZ_CP025268.1, 1964878, 1966611, +, 1734, 123037; cds-WP_211180499.1, NZ_CP025268.1, 1966653, 1966697, -, 45, 669; cds-WP_001299676.1, NZ_CP025268.1, 1966788, 1967276, +, 489, 4186; cds-WP _001259583.1, NZ_CP025268.1, 1967396, 1967788, -, 393, 184; cds-WP_000066951.1, NZ_CP025268.1, 1967788, 1969866, -, 2079, 320333; cds-WP_001278954.1, NZ_CP025268.1, 1969859, 1971007, -, 1149, 72; cds-WP_000983609.1, NZ_CP025268.1, 1971209, 1971853, -, 645, 8631; cds-WP_000763867.1, NZ_CP025268.1, 1971864, 1972253, -, 390, 17615; cds-WP _000036361.1, NZ_CP025268.1, 1972268, 1973317, -, 1050, 301052; cds-WP _000204337.1, NZ_CP025268.1, 1973320, 1974180, -, 861, 32114; cds-WP _000483239.1, NZ_CP025268.1, 1974199, 1975800, -, 1602, 314159; cds-WP _001297437.1, NZ_CP025268.1, 1975846, 1977507, -, 1662, 17650; cds-WP_000147302.1, NZ_CP025268.1, 1977652, 1978155, -, 504, 772; cds-
WP_001350517.1, NZ_CP025268.1, 1978176, 1980140, -, 1965, 574532; cds-WP _000795630.1, NZ_CP025268.1, 1980145, 1981071, -, 927, 423235; cds-WP _000906340.1, NZ_CP025268.1, 1981068, 1981955, -, 888, 231158; cds-WP_001291603.1, NZ_CP025268.1, 1982082, 1982660, -, 579, 6608; cds-WP_001295647.1, NZ_CP025268.1, 1982663, 1983013, -, 351, 3220; cds-WP _000122416.1, NZ_CP025268.1, 1983793, 1984221, +, 429, 0; cds-WP_001295646.1, NZ_CP025268.1, 1984228, 1985652, -, 1425, 59; cds-WP_001295645.1, NZ_CP025268.1, 1985627, 1986427, -, 801, 472; cds-WP _000100205.1, NZ_CP025268.1, 1986594, 1987580, -, 987, 50; cds-WP_001187819.1, NZ_CP025268.1, 1987595, 1989109, -, 1515, 0; cds-WP _000548675.1, NZ_CP025268.1, 1989179, 1990168, -, 990, 0; cds-WP _000179469.1, NZ_CP025268.1, 1990965, 1991468, +, 504, 12; cds-WP_000082127.1, NZ_CP025268.1, 1991547, 1991798, -, 252, 1; cds-WP_010723106.1, NZ_CP025268.1, 1991913, 1991999, -, 87, 1; cds-WP_001237869.1, NZ_CP025268.1, 1992262, 1992585, +, 324, 3; cds-WP_000917208.1, NZ_CP025268.1, 1992756, 1993253, +, 498, 47261; cds-WP _000377225.1, NZ_CP025268.1, 1993291, 1993530, -, 240, 0; cds-WP_000797560.1, NZ_CP025268.1, 1993721, 1994932, +, 1212, 33843; cds-WP_000847902.1, NZ_CP025268.1, 1994994, 1995659, -, 666, 46250; cds-WP _001160187.1, NZ_CP025268.1, 1996309, 1996857, -, 549, 0; cds-WP _001283421.1, NZ_CP025268.1, 1996914, 1998746, -, 1833, 244575; cds-WP _000611335.1, NZ_CP025268.1, 1998743, 1999399, -, 657, 16084; cds-WP_000590347.1, NZ_CP025268.1, 1999695, 1999871, +, 177, 23; cds-WP_000106474.1, NZ_CP025268.1, 1999858, 2000082, +, 225, 11; cds-WP_001152715.1, NZ_CP025268.1, 2000150, 2000872, -, 723, 12292; cds-WP _001272991.1, NZ_CP025268.1, 2001102, 2001854, -, 753, 68628; cds-WP _001158220.1, NZ_CP025268.1, 2001851, 2002519, -, 669, 4249; cds-WP _001128215.1, NZ_CP025268.1, 2002534, 2003520, -, 987, 23; cds-WP _001296168.1, NZ_CP025268.1, 2003625, 2004425, -, 801, 39753; cds-WP _001350518.1, NZ_CP025268.1, 2004513, 2005064, -, 552, 252496; cds-WP_001087467.1, NZ_CP025268.1, 2005110, 2005829, -, 720, 179333; cds-WP_000079739.1, NZ_CP025268.1, 2006150, 2007646, -, 1497, 2442576; cds-WP_000146784.1, NZ_CP025268.1, 2007912, 2009318, +, 1407, 248527; cds-WP _000270671.1, NZ_CP025268.1, 2009343, 2009753, +, 411, 45043; cds-WP_001015033.1, NZ_CP025268.1, 2009753, 2010118, +, 366, 5302; cds-WP _001245695.1, NZ_CP025268.1, 2010196, 2011683, +, 1488, 65371; cds-WP _001350519.1, NZ_CP025268.1, 2011717, 2012130, -, 414, 4632; cds-WP _000118897.1, NZ_CP025268.1, 2012317, 2013522, +, 1206, 19861; cds-WP_000790504.1, NZ_CP025268.1, 2013519, 2013752, +, 234, 11; cds-WP_000334583.1, NZ_CP025268.1, 2013861, 2014529, +, 669, 0; cds-WP _000494172.1, NZ_CP025268.1, 2014640, 2015119, +, 480, 19513; cds-WP _001362554.1, NZ_CP025268.1, 2015388, 2015579, -, 192, 1784; cds-CXP41_RS10705, NZ_CP025268.1, 2015589, 2016391, -, 803, 68548; cds-WP_001274299.1, NZ_CP025268.1, 2016740, 2017054, -, 315, 1393; cds-WP _000994427.1, NZ_CP025268.1, 2017269, 2018927, +, 1659, 116023; cds-WP _000067950.1, NZ_CP025268.1, 2018920, 2019915, +, 996, 303259; cds-WP _001282683.1, NZ_CP025268.1, 2019908, 2020594, +, 687, 29965; cds-WP_000213294.1, NZ_CP025268.1, 2020594, 2021967, +, 1374, 511687; cds-WP _000807579.1, NZ_CP025268.1, 2021986, 2022429, +, 444, 90634; cds-WP_000620071.1, NZ_CP025268.1, 2022426, 2023553, +, 1128, 48053; cds-WP_000133106.1, NZ_CP025268.1, 2023658, 2024122, +, 465, 142261; cds-WP_001350520.1, NZ_CP025268.1, 2024127, 2025131, +, 1005, 653829; cds-WP _001282101.1, NZ_CP025268.1, 2025128, 2025541, +, 414, 97349; cds-WP_001302429.1, NZ_CP025268.1, 2025544, 2025909, +, 366, 351875; cds-WP_001253441.1, NZ_CP025268.1, 2025909, 2026646, +, 738, 107785; cds-WP _000187358.1, NZ_CP025268.1, 2026656, 2026925, +, 270, 105328; cds-WP_000942319.1, NZ_CP025268.1, 2026933, 2027718, +, 786, 142296; cds-WP_000104001.1, NZ_CP025268.1, 2028008, 2028631, +, 624, 14; cds-WP _000867217.1, NZ_CP025268.1, 2028675, 2028863, -, 189, 0; cds-WP _000844800.1, NZ_CP025268.1, 2029026, 2029253, +, 228, 5; cds-WP_000491520.1, NZ_CP025268.1, 2029551, 2030366, +, 816, 7; cds-WP _001350521.1, NZ_CP025268.1, 2030363, 2032057, -, 1695, 335800; cds-WP _000009307.1, NZ_CP025268.1, 2032228, 2032410, -, 183, 0; cds-WP_000922686.1, NZ_CP025268.1, 2032489, 2033406, -, 918, 3; cds-CXP41_RS10830, NZ_CP025268.1, 2033579, 2034498, +, 920, 149346; cds-WP _000786005.1, NZ_CP025268.1, 2034487, 2034957, -, 471, 29891; cds-WP _001157239.1, NZ_CP025268.1, 2034938, 2036356, -, 1419, 331913; cds-WP _000365562.1, NZ_CP025268.1, 2036423, 2037118, -, 696, 3629; cds-WP_001313057.1, NZ_CP025268.1, 2037158, 2037523, -, 366, 0; cds-CXP41_RS10855, NZ_CP025268.1, 2038090, 2039282, +, 1193, 26; cds-WP _000218212.1, NZ_CP025268.1, 2039874, 2040725, +, 852, 82; cds-WP _000826792.1, NZ_CP025268.1, 2040833, 2042191, -, 1359, 901; cds-WP_001395354.1, NZ_CP025268.1, 2042191, 2042862, -, 672, 12082; cds-WP_000920120.1, NZ_CP025268.1, 2042995, 2043408, +, 414, 0; cds-WP_000740106.1, NZ_CP025268.1, 2043517, 2044521, +, 1005, 0; cds-WP_001240091.1, NZ_CP025268.1, 2044522, 2045157, +, 636, 0; cds-WP _001007805.1, NZ_CP025268.1, 2045414, 2046064, +, 651, 0; cds-WP_001079074.1, NZ_CP025268.1, 2046407, 2046937, +, 531, 5174; cds-WP_001302302.1, NZ_CP025268.1, 2047690, 2048487, +, 798, 0; cds-WP_157825727.1, NZ_CP025268.1, 2048977, 2056053, +, 7077, 62990; cds-CXP41_RS10930, NZ_CP025268.1, 2056315, 2057367, -, 1053, 0; cds-WP _000378575.1, NZ_CP025268.1, 2057682, 2058998, +, 1317, 19469; cds-WP _001060244.1, NZ_CP025268.1, 2059100, 2060554, +, 1455, 188343; cds-WP _000532923.1, NZ_CP025268.1, 2060897, 2061613, +, 717, 6206; cds-WP_000943916.1, NZ_CP025268.1, 2062242, 2063885, -, 1644, 0; cds-WP_001011008.1, NZ_CP025268.1, 2064003, 2064953, -, 951, 701; cds-WP_001011462.1, NZ_CP025268.1, 2065055, 2065972, -, 918, 0; cds-WP _000986321.1, NZ_CP025268.1, 2066430, 2067365, -, 936, 25096; cds-WP _001166160.1, NZ_CP025268.1, 2067427, 2068506, -, 1080, 29955; cds-WP _001326708.1, NZ_CP025268.1, 2068518, 2069261, -, 744, 353; cds-WP_000973176.1, NZ_CP025268.1, 2069258, 2069803, -, 546, 3832; cds-WP_000019402.1, NZ_CP025268.1, 2070344, 2071324, -, 981, 189892; cds-CXP41_RS11035, NZ_CP025268.1, 2072674, 2072979, +, 306, 7; cds-WP_088895425.1-3, NZ_CP025268.1;NZ_CP025268.1, 2072991;2073904, 2073904;2074219, -;-, 1229, 8621; cds-CXP41_RS11045, NZ_CP025268.1, 2074313, 2074510, +, 198, 0; cds-WP_014639038.1, NZ_CP025268.1, 2074540, 2075250, +, 711, 0; cds-WP_101348613.1, NZ_CP025268.1, 2075578, 2078697, +, 3120, 10516; cds-WP _001350525.1, NZ_CP025268.1, 2078818, 2080350, +, 1533, 44496; cds-WP _000187523.1, NZ_CP025268.1, 2080347, 2080793, +, 447, 0; cds-WP_000692323.1, NZ_CP025268.1, 2080856, 2081077, +, 222, 0; cds-WP _001285584.1, NZ_CP025268.1, 2081151, 2081519, +, 369, 0; cds-WP _000854814.1, NZ_CP025268.1, 2081608, 2081982, +, 375, 0; cds-WP_000988600.1, NZ_CP025268.1, 2081979, 2082173, +, 195, 0; cds-WP _001161660.1, NZ_CP025268.1, 2082186, 2082299, +, 114, 0; cds-CXP41_RS11090, NZ_CP025268.1, 2082588, 2082716, -, 129, 0; cds-WP_001016348.1, NZ_CP025268.1, 2082788, 2082970, +, 183, 0; cds-WP _000450409.1, NZ_CP025268.1, 2083071, 2083400, -, 330, 6805; cds-WP_001200891.1, NZ_CP025268.1, 2083572, 2084630, -, 1059, 6; cds-WP_001105415.1, NZ_CP025268.1, 2084828, 2085301, -, 474, 88974; cds-WP _000830156.1, NZ_CP025268.1, 2085420, 2086586, -, 1167, 10679; cds-WP_000980589.1, NZ_CP025268.1, 2086795, 2088222, +, 1428, 1; cds-WP_000234896.1, NZ_CP025268.1, 2088265, 2088492, -, 228, 162; cds-WP_000492339.1, NZ_CP025268.1, 2088506, 2089564, -, 1059, 6; cds-WP_101348614.1, NZ_CP025268.1, 2089743, 2091101, -, 1359, 372683; cds-WP_010723108.1, NZ_CP025268.1, 2091091, 2091153, -, 63, 1514; cds-WP_000803351.1, NZ_CP025268.1, 2091368, 2092297, -, 930, 229; cds-WP_000754737.1, NZ_CP025268.1, 2092343, 2093167, -, 825, 49299; cds-WP _000767829.1, NZ_CP025268.1, 2093250, 2093504, -, 255, 73730; cds-WP _001259255.1, NZ_CP025268.1, 2093501, 2093752, -, 252, 35696; cds-WP _001364200.1, NZ_CP025268.1, 2094035, 2094085, +, 51, 76; cds-WP_000131782.1, NZ_CP025268.1, 2094231, 2095130, +, 900, 7199; cds-WP _000009594.1, NZ_CP025268.1, 2095136, 2096440, +, 1305, 59; cds-WP_000108941.1, NZ_CP025268.1, 2096437, 2097507, +, 1071, 4059; cds-WP _000080105.1, NZ_CP025268.1, 2097507, 2098574, +, 1068, 46591; cds-WP_001103560.1, NZ_CP025268.1, 2098574, 2099164, +, 591, 72334; cds-WP_000586462.1, NZ_CP025268.1, 2099164, 2099901, +, 738, 603; cds-WP_000880182.1, NZ_CP025268.1, 2099883, 2100659, +, 777, 346209; cds-WP _000954911.1, NZ_CP025268.1, 2100653, 2101264, +, 612, 72119; cds-WP _001393575.1, NZ_CP025268.1, 2101360, 2102340, -, 981, 232026; cds-WP_101348615.1, NZ_CP025268.1, 2102486, 2103652, -, 1167, 25336; cds-WP_000043484.1, NZ_CP025268.1, 2103901, 2105307, -, 1407, 1579997; cds-CXP41_RS11230, NZ_CP025268.1, 2105435, 2105788, -, 354, 18028; cds-WP_000019403.1-5, NZ_CP025268.1, 2105934, 2106914, -, 981, 96838; cds-CXP41_RS11245, NZ_CP025268.1, 2106985, 2107428, -, 444, 56891; cds-WP_101348616.1, NZ_CP025268.1, 2107430, 2108548, -, 1119, 614798; cds-WP _000601187.1, NZ_CP025268.1, 2108533, 2109123, -, 591, 103462; cds-WP_001407542.1, NZ_CP025268.1, 2109104, 2110096, -, 993, 146180; cds-WP_000639866.1, NZ_CP025268.1, 2110099, 2111265, -, 1167, 741782; cds-WP_000272486.1, NZ_CP025268.1, 2111265, 2112368, -, 1104,724072; cds-WP_001393538.1, NZ_CP025268.1, 2112376, 2113623, -, 1248, 659655; cds-WP_001100981.1, NZ_CP025268.1, 2113620, 2114177, -, 558, 278096; cds-WP_000783975.1, NZ_CP025268.1, 2114177, 2115058, -, 882, 692185; cds-WP _001023610.1, NZ_CP025268.1, 2115116, 2116015, -, 900, 1610962; cds-WP _000699460.1, NZ_CP025268.1, 2116015, 2117100, -, 1086, 762907; cds-WP _000183060.1, NZ_CP025268.1, 2117473, 2118366, -, 894, 72839; cds-WP_001116026.1, NZ_CP025268.1, 2118541, 2119935, -, 1395, 0; cds-WP_000862592.1, NZ_CP025268.1, 2119946, 2121166, -, 1221, 5; cds-WP_101348617.1, NZ_CP025268.1, 2121163, 2122443, -, 1281, 0; cds-WP_000058424.1, NZ_CP025268.1, 2122719, 2124197, -, 1479, 0; cds-WP _000183107.1, NZ_CP025268.1, 2124199, 2125593, -, 1395, 0; cds-WP_001350528.1, NZ_CP025268.1, 2125648, 2127018, -, 1371, 0; cds-WP_000079274.1, NZ_CP025268.1, 2127123, 2128559, -, 1437, 0; cds-WP _000699693.1, NZ_CP025268.1, 2128562, 2129785, -, 1224, 0; cds-WP _001393539.1, NZ_CP025268.1, 2129782, 2130261, -, 480, 0; cds-WP _000043654.1, NZ_CP025268.1, 2130264, 2131229, -, 966, 0; cds-WP 000048190.1, NZ_CP025268.1, 2131232, 2132353, -, 1122, 0; cds-WP_001153547.1, NZ_CP025268.1, 2132379, 2132927, -, 549, 0; cds-WP _000927089.1, NZ_CP025268.1, 2132943, 2133689, -, 747, 0; cds-WP_101348618.1, NZ_CP025268.1, 2133700, 2134917, -, 1218, 0; cds-WP_101348619.1, NZ_CP025268.1, 2134892, 2136109, -, 1218, 0; cds-WP_000888740.1, NZ_CP025268.1, 2136106, 2136594, -, 489, 0; cds-WP_000794699.1, NZ_CP025268.1, 2136597, 2137436, -, 840,11803; cds-WP_000137196.1, NZ_CP025268.1, 2137529, 2139691, -, 2163, 8086; cds-WP_000482901.1, NZ_CP025268.1, 2139694, 2140137, -, 444, 0; cds-WP_000978094.1, NZ_CP025268.1, 2140143, 2141282, -, 1140, 0; cds-WP_000454701.1, NZ_CP025268.1, 2141941, 2143524, +, 1584, 10074; cds-WP_001252331.1, NZ_CP025268.1, 2143798, 2145651, -, 1854, 480531; cds-WP_001234768.1, NZ_CP025268.1, 2145673, 2146254, -, 582, 66104; cds-WP _001295424.1, NZ_CP025268.1, 2146346, 2146987, -, 642, 73641; cds-WP_000043761.1, NZ_CP025268.1, 2147305, 2150622, +, 3318, 61; cds-WP _000288420.1, NZ_CP025268.1, 2150731, 2151579, -, 849, 5; cds-WP _000469708.1, NZ_CP025268.1, 2151713, 2153065, +, 1353, 15833; cds-WP_000856081.1, NZ_CP025268.1, 2153078, 2155024, -, 1947, 20565; cds-WP _000722348.1, NZ_CP025268.1, 2155224, 2155685, +, 462, 0; cds-WP_000119090.1, NZ_CP025268.1, 2155750, 2156511, -, 762, 0; cds-WP_000003154.1, NZ_CP025268.1, 2156508, 2157167, -, 660, 0; cds-WP_010723109.1, NZ_CP025268.1, 2157388, 2157447, -, 60, 0; cds-WP _001386899.1, NZ_CP025268.1, 2157720, 2157776, -, 57, 0; cds-WP_000678989.1, NZ_CP025268.1, 2158055, 2159302, +, 1248, 33675; cds-WP_001197875.1, NZ_CP025268.1, 2159302, 2162424, +, 3123, 528; cds-WP_000667481.1, NZ_CP025268.1, 2162425, 2165502, +, 3078, 0; cds-WP _000130850.1, NZ_CP025268.1, 2165503, 2166918, +, 1416, 24; cds-WP _000675150.1, NZ_CP025268.1, 2166915, 2168318, +, 1404, 5; cds-WP _000137877.1, NZ_CP025268.1, 2168315, 2169037, +, 723, 316; cds-WP _001350529.1, NZ_CP025268.1, 2169228, 2169560, +, 333, 0; cds-WP _000476011.1, NZ_CP025268.1, 2169707, 2171068,
+, 1362, 75524; cds-WP _000468310.1, NZ_CP025268.1, 2171341, 2171559, -, 219, 0; cds-CXP41_RS11525, NZ_CP025268.1, 2171641, 2171853, -, 213, 0; cds-WP _001318299.1, NZ_CP025268.1, 2172028, 2172345, -, 318, 0; cds-WP_000807348.1, NZ_CP025268.1, 2172751, 2173650, +, 900, 0; cds-CXP41_RS11545, NZ_CP025268.1, 2173732, 2174208, -, 477, 0; cds-WP _085947771.1-4, NZ_CP025268.1;NZ_CP025268.1, 2174275;2174536, 2174536;2175437, +;+, 1163, 6; cds-CXP41_RS11555, NZ_CP025268.1, 2175467, 2175766, -, 300, 0; cds-WP_000844219.1, NZ_CP025268.1, 2175872, 2176912, -, 1041, 0; cds-WP _000490679.1, NZ_CP025268.1, 2176960, 2178315, -, 1356, 134433; cds-WP _000823270.1, NZ_CP025268.1, 2178319, 2178603, -, 285, 3865; cds-WP _000182899.1, NZ_CP025268.1, 2178634, 2179086, -, 453, 22; cds-WP _000853883.1, NZ_CP025268.1, 2179096, 2180358, -, 1263, 54874; cds-WP_001307281.1, NZ_CP025268.1, 2180387, 2181241, -, 855, 427067; cds-WP_000129551.1, NZ_CP025268.1, 2181549, 2182601, -, 1053, 0; cds-WP_000858484.1, NZ_CP025268.1, 2182858, 2184135, +, 1278, 27; cds-WP_000846217.1, NZ_CP025268.1, 2184132, 2185136, +, 1005, 2; cds-WP_000012001.1, NZ_CP025268.1, 2185133, 2186098, +, 966, 0; cds-WP_000434038.1, NZ_CP025268.1, 2186072, 2186818, -, 747, 2; cds-WP _001351783.1, NZ_CP025268.1, 2186870, 2187688, -, 819, 0; cds-WP_000822274.1, NZ_CP025268.1, 2187753, 2188553, -, 801, 21751; cds-WP _001195564.1, NZ_CP025268.1, 2188550, 2189338, -, 789, 68376; cds-WP _000019944.1, NZ_CP025268.1, 2189561, 2189833, -, 273, 0; cds-WP_000134636.1, NZ_CP025268.1, 2189954, 2190778, +, 825, 0; cds-WP_000153067.1, NZ_CP025268.1, 2190997, 2191335, +, 339, 5; cds-WP _000405713.1, NZ_CP025268.1, 2191417, 2192451, -, 1035, 0; cds-WP_000945454.1, NZ_CP025268.1, 2192467, 2194947, -, 2481, 9; cds-WP _000677393.1, NZ_CP025268.1, 2194963, 2195637, -, 675, 0; cds-WP_000830456.1, NZ_CP025268.1, 2195717, 2196259, -, 543, 0; cds-WP_001324851.1, NZ_CP025268.1, 2196552, 2196833, -, 282, 16745; cds-WP_001005448.1, NZ_CP025268.1, 2197096, 2198205, -, 1110, 146358; cds-WP _001350533.1, NZ_CP025268.1, 2198337, 2200370, +, 2034, 637547; cds-CXP41_RS24230, NZ_CP025268.1, 2200511, 2204306, +, 3796, 10830; cds-WP _001373533.1, NZ_CP025268.1, 2204316, 2204558, +, 243, 0; cds-WP_101348620.1, NZ_CP025268.1, 2204555, 2207947, +, 3393, 398; cds-WP_000636925.1, NZ_CP025268.1, 2208008, 2208325, +, 318, 0; cds-WP_001350535.1, NZ_CP025268.1, 2208632, 2209720, +, 1089, 0; cds-WP _001294387.1, NZ_CP025268.1, 2209731, 2212010, +, 2280, 0; cds-WP_001333512.1, NZ_CP025268.1, 2212003, 2213139, +, 1137, 0; cds-CXP41_RS11730, NZ_CP025268.1, 2213136, 2215136, +, 2001, 108; cds-WP _001295429.1, NZ_CP025268.1, 2215261, 2215722, +, 462, 9720; cds-WP _000950409.1, NZ_CP025268.1, 2215762, 2216232, -, 471, 9462; cds-WP _000598641.1, NZ_CP025268.1, 2216279, 2216998, -, 720, 4; cds-CXP41_RS11750, NZ_CP025268.1, 2216995, 2218679, -, 1685, 0; cds-WP_001240401.1, NZ_CP025268.1, 2218901, 2219632, +, 732, 0; cds-WP_001216963.1, NZ_CP025268.1, 2219692, 2219799, +, 108, 0; cds-WP _000783123.1, NZ_CP025268.1, 2219780, 2220511, -, 732, 6999; cds-WP_000569315.1, NZ_CP025268.1, 2220516, 2221442, -, 927, 0; cds-WP_000220837.1, NZ_CP025268.1, 2221435, 2222592, -, 1158, 0; cds-WP_001130308.1, NZ_CP025268.1, 2222599, 2223516, -, 918, 41544; cds-WP_069905050.1, NZ_CP025268.1, 2223727, 2226024, -, 2298, 522442; cds-WP _000097403.1, NZ_CP025268.1, 2226220, 2227935, +, 1716, 32203; cds-WP_001319943.1, NZ_CP025268.1, 2227973, 2228905, -, 933, 1082; cds-WP _001295454.1, NZ_CP025268.1, 2229079, 2229666, -, 588, 0; cds-WP _001296821.1, NZ_CP025268.1, 2229836, 2230414, +, 579, 17; cds-WP_000079538.1, NZ_CP025268.1, 2230544, 2231305, -, 762, 0; cds-CXP41_RS11815, NZ_CP025268.1, 2231358, 2232793, -, 1436, 256; cds-WP _000691708.1, NZ_CP025268.1, 2233017, 2233100, +, 84, 0; cds-WP_001264861.1, NZ_CP025268.1, 2233473, 2234420, -, 948, 36; cds-WP _001295452.1, NZ_CP025268.1, 2234659, 2235057, +, 399, 1; cds-WP_000968208.1, NZ_CP025268.1, 2235054, 2235749, +, 696, 5; cds-WP_000553555.1, NZ_CP025268.1, 2235879, 2236763, +, 885, 19; cds-WP_000920064.1, NZ_CP025268.1, 2236913, 2237632, +, 720, 11065; cds-WP _000383096.1, NZ_CP025268.1, 2237635, 2237874, +, 240, 1; cds-WP_001136389.1, NZ_CP025268.1, 2238068, 2239306, +, 1239, 0; cds-WP_000956071.1, NZ_CP025268.1, 2239300, 2240535, +, 1236, 0; cds-WP_001275118.1, NZ_CP025268.1, 2240778, 2241788, -, 1011, 0; cds-WP _000255039.1, NZ_CP025268.1, 2241804, 2243324, -, 1521, 40; cds-WP _001036964.1, NZ_CP025268.1, 2243385, 2244383, -, 999, 0; cds-CXP41_RS11890, NZ_CP025268.1, 2244663, 2245704, -, 1042, 8; cds-WP_000440926.1, NZ_CP025268.1, 2245846, 2247003, -, 1158, 7203; cds-WP _001139613.1, NZ_CP025268.1, 2247020, 2247688, -, 669, 241878; cds-WP_000425438.1, NZ_CP025268.1, 2247946, 2248782, +, 837, 16411; cds-WP_000489247.1, NZ_CP025268.1, 2248814, 2250805, -, 1992, 128422; cds-WP _000253273.1, NZ_CP025268.1, 2251099, 2252568, -, 1470, 74012; cds-WP _000548294.1, NZ_CP025268.1, 2252773, 2253654, -, 882, 14; cds-WP_000182053.1, NZ_CP025268.1, 2253753, 2254802, +, 1050, 171; cds-WP _000873894.1, NZ_CP025268.1, 2254876, 2255733, +, 858, 59013; cds-WP_001065016.1, NZ_CP025268.1, 2255736, 2256824, +, 1089, 24; cds-WP _000382966.1, NZ_CP025268.1, 2256931, 2258181, -, 1251, 647; cds-WP_000415429.1, NZ_CP025268.1, 2258281, 2259222, -, 942, 0; cds-WP_000658575.1, NZ_CP025268.1, 2259352, 2260050, +, 699, 0; cds-WP _000353878.1, NZ_CP025268.1, 2260121, 2261371, -, 1251, 0; cds-WP _001292460.1, NZ_CP025268.1, 2261465, 2262403, -, 939, 0; cds-WP _001208118.1, NZ_CP025268.1, 2262391, 2263332, -, 942, 0; cds-WP_000854447.1, NZ_CP025268.1, 2263755, 2265446, -, 1692, 522789; cds-WP _000091263.1, NZ_CP025268.1, 2265463, 2266401, -, 939, 285075; cds-WP _000487246.1, NZ_CP025268.1, 2266401, 2267531, -, 1131, 75977; cds-WP_000551980.1, NZ_CP025268.1, 2267899, 2269080, +, 1182, 0; cds-WP_000389030.1, NZ_CP025268.1, 2269077, 2269331, -, 255, 0; cds-WP_001136827.1, NZ_CP025268.1, 2269486, 2270058, +, 573, 1098; cds-WP _001091940.1, NZ_CP025268.1, 2270281, 2271747, +, 1467, 225689; cds-WP_000198828.1, NZ_CP025268.1, 2271865, 2272851, +, 987, 44; cds-WP_000594599.1, NZ_CP025268.1, 2272890, 2273603, +, 714, 34818; cds-WP _000241011.1, NZ_CP025268.1, 2274015, 2274581, +, 567, 125896; cds-WP_000470560.1, NZ_CP025268.1, 2274762, 2276318, +, 1557, 96952; cds-WP_000637046.1, NZ_CP025268.1, 2276400, 2278214, +, 1815, 5; cds-WP _000501604.1, NZ_CP025268.1, 2278215, 2279309, +, 1095, 12027; cds-WP_000088923.1, NZ_CP025268.1, 2279309, 2280334, +, 1026, 87543; cds-WP_000194927.1, NZ_CP025268.1, 2280336, 2281925, +, 1590, 123153; cds-WP_000202798.1, NZ_CP025268.1, 2281929, 2282273, -, 345, 19501; cds-WP _000213361.1, NZ_CP025268.1, 2282606, 2283796, -, 1191, 59460; cds-WP _001234850.1, NZ_CP025268.1, 2283824, 2284519, -, 696, 11500; cds-WP_000578061.1, NZ_CP025268.1, 2284668, 2286428, +, 1761, 15; cds-WP _000494183.1, NZ_CP025268.1, 2286553, 2286837, +, 285, 259862; cds-WP _000050793.1, NZ_CP025268.1, 2286976, 2287983, -, 1008, 1016; cds-WP_001135667.1, NZ_CP025268.1, 2288165, 2288392, +, 228, 50009; cds-WP_000256203.1, NZ_CP025268.1, 2288412, 2290172, +, 1761, 936850; cds-CXP41_RS12130, NZ_CP025268.1, 2290426, 2292956, -, 2531, 59713; cds-WP_000019403.1-6, NZ_CP025268.1, 2293100, 2294080, -, 981, 106606; cds-WP_001113639.1, NZ_CP025268.1, 2294535, 2295182, +, 648, 19; cds-WP _001211575.1, NZ_CP025268.1, 2295393, 2296445, -, 1053, 18216; cds-WP _000824439.1, NZ_CP025268.1, 2296442, 2296999, -, 558, 37; cds-WP_000982447.1, NZ_CP025268.1, 2296996, 2298939, -, 1944, 2775; cds-WP _001026418.1, NZ_CP025268.1, 2298936, 2299415, -, 480, 176; cds-WP _000186540.1, NZ_CP025268.1, 2299412, 2299621, -, 210, 0; cds-WP_001295447.1, NZ_CP025268.1, 2299618, 2300355, -, 738, 0; cds-WP_000971730.1, NZ_CP025268.1, 2300397, 2301059, -, 663, 1; cds-WP_000525587.1, NZ_CP025268.1, 2301056, 2301679, -, 624, 18867; cds-WP _000528376.1, NZ_CP025268.1, 2301692, 2302294, -, 603, 3; cds-WP _000835174.1, NZ_CP025268.1, 2302304, 2302753, -, 450, 0; cds-WP _000013515.1, NZ_CP025268.1, 2302750, 2303613, -, 864, 48407; cds-WP _000091291.1, NZ_CP025268.1, 2303600, 2304295, -, 696, 1987; cds-WP_000778061.1, NZ_CP025268.1, 2304302, 2306788, -, 2487, 0; cds-WP_000557378.1, NZ_CP025268.1, 2306785, 2307048, -, 264, 0; cds-WP_000686723.1, NZ_CP025268.1, 2307038, 2307532, -, 495, 0; cds-WP_001310984.1, NZ_CP025268.1, 2307641, 2307805, +, 165, 0; cds-WP _000849209.1, NZ_CP025268.1, 2307940, 2308428, +, 489, 3813; cds-WP _000758077.1, NZ_CP025268.1, 2309143, 2310789, -, 1647, 168934; cds-WP _000422182.1, NZ_CP025268.1, 2311007, 2312650, -, 1644, 461733; cds-WP _000884971.1, NZ_CP025268.1, 2312726, 2313376, -, 651, 0; cds-WP _000710375.1, NZ_CP025268.1, 2313376, 2314440, -, 1065, 382308; cds-WP _000406064.1, NZ_CP025268.1, 2314514, 2315569, -, 1056, 64199; cds-WP_000865568.1, NZ_CP025268.1, 2315681, 2316784, -, 1104, 2637154; cds-WP _001249081.1, NZ_CP025268.1, 2317523, 2320195, +, 2673, 880544; cds-WP _001061917.1, NZ_CP025268.1, 2320212, 2320862, +, 651, 107674; cds-WP_101348621.1, NZ_CP025268.1, 2321062, 2323911, -, 2850,137816; cds-WP_000559125.1, NZ_CP025268.1, 2324078, 2325904, +, 1827, 3; cds-WP_000125282.1, NZ_CP025268.1, 2325901, 2327286, +, 1386, 0; cds-WP _000850540.1, NZ_CP025268.1, 2327482, 2328144, +, 663, 0; cds-WP_000339065.1, NZ_CP025268.1, 2328144, 2328794, +, 651, 0; cds-WP_000580275.1, NZ_CP025268.1, 2328791, 2330113, +, 1323, 0; cds-WP _000786547.1, NZ_CP025268.1, 2330144, 2331328, +, 1185, 0; cds-WP_001225852.1, NZ_CP025268.1, 2331402, 2332178, -, 777, 0; cds-WP _001104549.1, NZ_CP025268.1, 2332183, 2333832, -, 1650, 0; cds-CXP41_RS12345, NZ_CP025268.1, 2333833, 2338347, -, 4515, 2; cds-WP_001295211.1, NZ_CP025268.1, 2338371, 2338994, -,624,0; cds-WP_000012305.1, NZ_CP025268.1, 2338991, 2340679, -, 1689, 6; cds-WP_001281242.1, NZ_CP025268.1, 2340828, 2343455, -, 2628, 1558803; cds-WP _000990765.1, NZ_CP025268.1, 2343602, 2344324, +, 723, 601; cds-WP_001220077.1, NZ_CP025268.1, 2344452, 2348204, -, 3753, 4319; cds-WP_001075164.1, NZ_CP025268.1, 2348900, 2351185, +, 2286, 1963785; cds-WP_211180501.1, NZ_CP025268.1, 2351385, 2351420, -, 36, 228887; cds-WP_000332037.1, NZ_CP025268.1, 2351419, 2352549, +, 1131,980744; cds-WP_000135040.1, NZ_CP025268.1, 2352549, 2352803, +, 255, 99236; cds-WP _000301050.1, NZ_CP025268.1, 2352857, 2353507, -, 651, 20470; cds-CXP41_RS12400, NZ_CP025268.1, 2353686, 2353928, +, 243, 0; cds-WP_000779105.1, NZ_CP025268.1, 2353970, 2355046, -, 1077, 0; cds-WP_000948731.1, NZ_CP025268.1, 2355051, 2356409, -, 1359, 0; cds-WP_000857257.1, NZ_CP025268.1, 2356682, 2358310, +, 1629, 282299; cds-WP _001209927.1, NZ_CP025268.1, 2358300, 2359559, +, 1260, 0; cds-WP_001000379.1, NZ_CP025268.1, 2359556, 2360746, +, 1191, 0; cds-WP_000140592.1, NZ_CP025268.1, 2360939, 2361838, +, 900, 33; cds-CXP41_RS12435, NZ_CP025268.1, 2361839, 2362036, +, 198, 886; cds-WP_088209894.1, NZ_CP025268.1, 2362077, 2362880, -, 804, 0; cds-WP_000100890.1, NZ_CP025268.1, 2362898, 2364187, -, 1290, 0; cds-WP_001319848.1, NZ_CP025268.1, 2364244, 2365449, -, 1206, 0; cds-WP_000894185.1, NZ_CP025268.1, 2365464, 2366246, -, 783, 0; cds-WP_000921621.1, NZ_CP025268.1, 2366466, 2367668, -, 1203, 142; cds-WP_001300392.1, NZ_CP025268.1, 2367768, 2368310, -, 543, 2; cds-WP _001300564.1, NZ_CP025268.1, 2368589, 2369014, +, 426, 0; cds-WP_000879112.1, NZ_CP025268.1, 2369053, 2369655, -, 603, 0; cds-WP _001295286.1, NZ_CP025268.1, 2369963, 2371102, +, 1140, 85081; cds-WP _000461657.1, NZ_CP025268.1, 2371106, 2372074, +, 969, 82; cds-WP_101348623.1, NZ_CP025268.1, 2372074, 2374065, +, 1992, 21; cds-WP_101348624.1, NZ_CP025268.1, 2374050, 2374943, +, 894, 0; cds-WP _000844057.1, NZ_CP025268.1, 2374943, 2376595, +, 1653, 13197; cds-WP _000638031.1, NZ_CP025268.1, 2376592, 2376927, +, 336, 9650; cds-WP_000523880.1, NZ_CP025268.1, 2376927, 2377313, +, 387, 1550; cds-WP_001295285.1, NZ_CP025268.1, 2377307, 2377573, -, 267, 0; cds-WP_000577625.1, NZ_CP025268.1, 2377683, 2379038, -, 1356, 49109; cds-WP_001255628.1, NZ_CP025268.1, 2379035, 2379997, -, 963, 26552; cds-WP_000639996.1, NZ_CP025268.1, 2379997, 2380854, -, 858, 360; cds-WP_000600499.1, NZ_CP025268.1, 2380869, 2381627, -, 759, 2006; cds-WP_001295284.1, NZ_CP025268.1, 2381624, 2383294, -, 1671, 234791; cds-WP _001191419.1, NZ_CP025268.1, 2383383, 2384678, -, 1296, 44341; cds-WP _000070621.1, NZ_CP025268.1, 2384757, 2385062, -, 306, 704; cds-WP_000574091.1, NZ_CP025268.1, 2385117,
2385578, -, 462, 1030; cds-WP _001300687.1, NZ_CP025268.1, 2385643, 2386560, +, 918, 119; cds-WP_001393504.1, NZ_CP025268.1, 2386748, 2387959, +, 1212, 551; cds-WP_001244806.1, NZ_CP025268.1, 2388030, 2389757, -, 1728, 0; cds-WP _000719999.1, NZ_CP025268.1, 2389895, 2390866, +, 972, 0; cds-WP _000525360.1, NZ_CP025268.1, 2390969, 2391472, +, 504, 0; cds-WP _000455114.1, NZ_CP025268.1, 2391745, 2392461, -, 717, 0; cds-WP_000188051.1, NZ_CP025268.1, 2392670, 2393092, +, 423, 0; cds-WP_000767280.1, NZ_CP025268.1, 2393151, 2393999, +, 849, 145549; cds-WP _000156701.1, NZ_CP025268.1, 2394083, 2395540, -, 1458, 403303; cds-WP _000926441.1, NZ_CP025268.1, 2395547, 2397076, -, 1530, 1119696; cds-WP _001056643.1, NZ_CP025268.1, 2397240, 2399081, -, 1842, 870993; cds-WP _000612644.1, NZ_CP025268.1, 2399078, 2399380, -, 303, 78787; cds-WP _000393511.1, NZ_CP025268.1, 2399377, 2399931, -, 555, 717066; cds-WP _000172749.1, NZ_CP025268.1, 2399943, 2400485, -, 543, 524948; cds-WP _000118507.1, NZ_CP025268.1, 2400500, 2401477, -, 978, 356088; cds-WP _000190939.1, NZ_CP025268.1, 2401474, 2404200, -, 2727, 1052505; cds-WP_000789500.1, NZ_CP025268.1, 2404253, 2405590, -, 1338, 795632; cds-WP _000545042.1, NZ_CP025268.1, 2405587, 2406087, -, 501, 238472; cds-WP _000247879.1, NZ_CP025268.1, 2406090, 2407892, -, 1803, 1213256; cds-WP _000386733.1, NZ_CP025268.1, 2407986, 2408648, -, 663, 385483; cds-WP _000062997.1, NZ_CP025268.1, 2408664, 2409107, -, 444, 269829; cds-WP_000622280.1, NZ_CP025268.1, 2409738, 2410676, -, 939, 47; cds-WP_000074527.1, NZ_CP025268.1, 2411596, 2412813, +, 1218, 509011; cds-WP_000813859.1, NZ_CP025268.1, 2412897, 2413496, +, 600, 0; cds-WP_001012899.1, NZ_CP025268.1, 2413555, 2415387, -, 1833, 182569; cds-WP _001203392.1, NZ_CP025268.1, 2415474, 2416124, -, 651, 37325; cds-WP _000426124.1, NZ_CP025268.1, 2416135, 2416629, -, 495, 8055; cds-WP_000106627.1, NZ_CP025268.1, 2416712, 2417167, -, 456, 91; cds-WP_000095707.1, NZ_CP025268.1, 2417505, 2418707, +, 1203, 722010; cds-WP_101348625.1, NZ_CP025268.1, 2418782, 2420926, +, 2145, 2101037; cds-WP_001274493.1, NZ_CP025268.1, 2421116, 2422636, +, 1521, 247146; cds-WP _000437935.1, NZ_CP025268.1, 2422669, 2423211, -, 543, 17927; cds-WP _000772452.1, NZ_CP025268.1, 2423269, 2423820, -, 552, 7903; cds-WP _000042442.1, NZ_CP025268.1, 2423876, 2424520, -, 645, 0; cds-WP_000566471.1, NZ_CP025268.1, 2424656, 2425303, +, 648, 0; cds-WP _000068457.1, NZ_CP025268.1, 2425360, 2425722, +, 363, 1174; cds-WP_001028341.1, NZ_CP025268.1, 2425743, 2426636, +, 894, 0; cds-WP _000156149.1, NZ_CP025268.1, 2426684, 2427574, -, 891, 2555; cds-WP_001293613.1, NZ_CP025268.1, 2427771, 2428544, -, 774, 46659; cds-WP_000569958.1, NZ_CP025268.1, 2428552, 2429268, -, 717, 5905; cds-WP _000965522.1, NZ_CP025268.1, 2429265, 2429951, -, 687, 233; cds-WP_000737621.1, NZ_CP025268.1, 2430041, 2430823, -, 783, 44; cds-WP_000748271.1, NZ_CP025268.1, 2431044, 2431826, -, 783, 125786; cds-WP _000825700.1, NZ_CP025268.1, 2432092, 2432661, -, 570, 24557; cds-WP _000334220.1, NZ_CP025268.1, 2432756, 2434273, -, 1518, 165527; cds-WP _000262111.1, NZ_CP025268.1, 2434310, 2434798, -, 489, 64623; cds-WP _000146992.1, NZ_CP025268.1, 2435057, 2435719, -, 663, 5851; cds-WP_000584546.1, NZ_CP025268.1, 2435709, 2436977, -, 1269, 381005; cds-WP_000118404.1, NZ_CP025268.1, 2437047, 2437961, -, 915, 210550; cds-WP_000364335.1, NZ_CP025268.1, 2438117, 2438776, -, 660, 12788; cds-WP_001283586.1, NZ_CP025268.1, 2438859, 2439671, -, 813, 143221; cds-WP _001289167.1, NZ_CP025268.1, 2439671, 2440684, -, 1014, 264232; cds-WP _000699148.1, NZ_CP025268.1, 2440750, 2441886, -, 1137, 283618; cds-WP_101348626.1, NZ_CP025268.1, 2441985, 2442980, +, 996, 5946; cds-WP _000127781.1, NZ_CP025268.1, 2442977, 2444155, -, 1179, 12888; cds-WP_000817178.1, NZ_CP025268.1, 2444420, 2445640, -, 1221, 2255396; cds-WP _000683799.1, NZ_CP025268.1, 2445799, 2447805, +, 2007, 27905; cds-WP_000559764.1, NZ_CP025268.1, 2447926, 2448204, -, 279, 107; cds-WP_001089222.1, NZ_CP025268.1, 2448238, 2448786, -, 549, 79979; cds-WP_000447361.1, NZ_CP025268.1, 2448786, 2449595, -, 810, 262905; cds-WP _001043825.1, NZ_CP025268.1, 2449595, 2450419, -, 825, 191488; cds-WP_001333535.1, NZ_CP025268.1, 2450423, 2451508, -, 1086, 174988; cds-WP_001300582.1, NZ_CP025268.1, 2451543, 2452475, -, 933, 124918; cds-WP_000730806.1, NZ_CP025268.1, 2452641, 2453192, +, 552, 10218; cds-WP_000698745.1, NZ_CP025268.1, 2453263, 2454084, -, 822, 13901; cds-WP_001043398.1, NZ_CP025268.1, 2454086, 2454625, -, 540, 1; cds-WP _001232541.1, NZ_CP025268.1, 2454622, 2455110, -, 489, 0; cds-WP_001311011.1, NZ_CP025268.1, 2455107, 2455619, -, 513, 0; cds-WP _001281606.1, NZ_CP025268.1, 2455619, 2456371, -, 753, 0; cds-CXP41_RS12915, NZ_CP025268.1, 2456391, 2459036, -, 2646, 19; cds-WP_000033316.1, NZ_CP025268.1, 2459118, 2459681, -, 564, 0; cds-WP _001195819.1, NZ_CP025268.1, 2460362, 2460847, -, 486, 0; cds-WP_000426176.1, NZ_CP025268.1, 2461050, 2463194, -, 2145, 0; cds-WP _000531952.1, NZ_CP025268.1, 2463194, 2464504, -, 1311, 0; cds-WP _001296261.1, NZ_CP025268.1, 2464685, 2464969, -, 285, 303; cds-WP_001295701.1, NZ_CP025268.1, 2465341, 2466681, +, 1341, 246832; cds-WP_000937788.1, NZ_CP025268.1, 2467046, 2468104, +, 1059, 335; cds-WP _000776765.1, NZ_CP025268.1, 2468286, 2469041, -, 756, 746; cds-WP_000368140.1, NZ_CP025268.1, 2469335, 2470267, +, 933, 0; cds-WP _000958671.1, NZ_CP025268.1, 2470579, 2471736, +, 1158, 33243; cds-WP _000915541.1, NZ_CP025268.1, 2471889, 2472251, +, 363, 318; cds-WP _000703651.1, NZ_CP025268.1, 2472248, 2473168, +, 921, 166932; cds-WP_001030215.1, NZ_CP025268.1, 2473165, 2474496, +, 1332, 19807; cds-WP_211180502.1, NZ_CP025268.1, 2474531, 2474677, -, 147, 27; cds-CXP41_RS13005, NZ_CP025268.1, 2474849, 2475139, +, 291, 6; cds-WP_101348627.1, NZ_CP025268.1, 2475111, 2475551, -, 441, 0; cds-WP_011443597.1, NZ_CP025268.1, 2475578, 2476096, -, 519, 0; cds-WP_000066913.1, NZ_CP025268.1, 2476146, 2476421, -, 276, 0; cds-WP_001393497.1, NZ_CP025268.1, 2476421, 2476915, -, 495, 0; cds-CXP41_RS24380, NZ_CP025268.1, 2476912, 2477619, -, 708, 0; cds-WP_000135673.1, NZ_CP025268.1, 2477638, 2478000, +, 363, 0; cds-WP_000081278.1, NZ_CP025268.1, 2478066, 2478890, +, 825, 0; cds-WP _000008183.1, NZ_CP025268.1, 2479018, 2479554, +, 537, 0; cds-WP _001242717.1, NZ_CP025268.1, 2479545, 2479907, +, 363, 901; cds-WP _000206809.1, NZ_CP025268.1, 2479907, 2480212, +, 306, 0; cds-WP_001163428.1, NZ_CP025268.1, 2480344, 2480544, +, 201, 0; cds-WP _001326971.1, NZ_CP025268.1, 2480728, 2481663, -, 936, 0; cds-WP_000556048.1, NZ_CP025268.1, 2481881, 2483218, +, 1338, 0; cds-WP_000426427.1, NZ_CP025268.1, 2483236, 2484564, +, 1329, 528; cds-WP _001018714.1, NZ_CP025268.1, 2484672, 2486210, -, 1539, 0; cds-WP _000435167.1, NZ_CP025268.1, 2486210, 2487373, -, 1164, 0; cds-WP _000991370.1, NZ_CP025268.1, 2487789, 2488403, +, 615, 30494; cds-CXP41_RS13095, NZ_CP025268.1, 2488408, 2492002, +, 3595, 107925; cds-WP_001296867.1, NZ_CP025268.1, 2492058, 2493203, -, 1146, 1; cds-WP_000955028.1, NZ_CP025268.1, 2493277, 2494221, -, 945, 0; cds-WP_001283490.1, NZ_CP025268.1, 2494291, 2495985, -, 1695, 0; cds-WP_000106759.1, NZ_CP025268.1, 2496039, 2497289, -, 1251, 0; cds-WP _000825600.1, NZ_CP025268.1, 2497802, 2498437, -, 636, 12; cds-WP_101348628.1, NZ_CP025268.1, 2498733, 2499008, +, 276, 0; cds-WP_000609275.1, NZ_CP025268.1, 2499085, 2499327, -, 243, 0; cds-WP _000484404.1, NZ_CP025268.1, 2499680, 2500600, +, 921, 33118; cds-WP _010723117.1, NZ_CP025268.1, 2500956, 2501027, +, 72, 0; cds-WP_000785931.1, NZ_CP025268.1, 2501092, 2502330, -, 1239, 294138; cds-WP_000544359.1, NZ_CP025268.1, 2502706, 2504403, +, 1698, 17; cds-WP _001295458.1, NZ_CP025268.1, 2504418, 2505152, +, 735, 11560; cds-WP _000646835.1, NZ_CP025268.1, 2505165, 2506022, +, 858, 24; cds-WP_000955906.1, NZ_CP025268.1, 2506025, 2508520, -, 2496, 0; cds-WP _000366028.1, NZ_CP025268.1, 2508545, 2509582, -, 1038, 0; cds-WP_000173288.1, NZ_CP025268.1, 2509582, 2510667, -, 1086, 0; cds-WP _000985359.1, NZ_CP025268.1, 2510682, 2511929, -, 1248, 0; cds-WP _000038456.1, NZ_CP025268.1, 2511951, 2512277, -, 327, 0; cds-WP_000170346.1, NZ_CP025268.1, 2512496, 2513461, -, 966, 904; cds-WP _000903148.1, NZ_CP025268.1, 2513665, 2514921, +, 1257, 3091; cds-WP _000490072.1, NZ_CP025268.1, 2515036, 2515362, +, 327, 73291; cds-WP _000186369.1, NZ_CP025268.1, 2515503, 2516741, -, 1239, 1900; cds-WP _000376337.1, NZ_CP025268.1, 2517077, 2518279, +, 1203, 96487; cds-WP_001300563.1-3, NZ_CP025268.1, 2518366, 2519478, +, 1113, 87645; cds-WP_0004974001, NZ_CP025268.1, 2519678, 2521867, -, 2190, 131624; cds-WP _000472021.1, NZ_CP025268.1, 2522487, 2522846, +, 360, 1698; cds-WP_000826503.1, NZ_CP025268.1, 2522848, 2523240, +, 393, 52588; cds-WP _000695655.1, NZ_CP025268.1, 2523292, 2524707, -, 1416, 481505; cds-WP_101348629.1, NZ_CP025268.1, 2525628, 2526512, -, 885, 221; cds-WP_000651752.1, NZ_CP025268.1, 2526553, 2526711, -, 159, 0; cds-WP_000020402.1, NZ_CP025268.1, 2526764, 2528020, -, 1257, 0; cds-WP_000084573.1, NZ_CP025268.1, 2528080, 2528913, -, 834, 0; cds-WP_000716421.1, NZ_CP025268.1, 2529162, 2529926, +, 765, 0; cds-WP_001109794.1, NZ_CP025268.1, 2529956, 2530882, -, 927, 0; cds-WP _000765610.1, NZ_CP025268.1, 2530972, 2531970, +, 999, 32; cds-WP _000410201.1, NZ_CP025268.1, 2531967, 2532185, -, 219, 0; cds-WP_000443661.1, NZ_CP025268.1, 2532187, 2534202, -, 2016, 48993; cds-WP_001300494.1, NZ_CP025268.1, 2534273, 2535259, -, 987, 23384; cds-WP_000254839.1, NZ_CP025268.1, 2535489, 2536250, +, 762, 15; cds-WP _000034402.1, NZ_CP025268.1, 2536435, 2537406, +, 972, 130076; cds-WP_000487600.1, NZ_CP025268.1, 2537790, 2538047, +, 258, 251220; cds-WP_000623140.1, NZ_CP025268.1, 2538092, 2539819, +, 1728, 2613075; cds-WP_000522247.1, NZ_CP025268.1, 2539860, 2540369, +, 510, 652770; cds-WP_000096674.1, NZ_CP025268.1, 2540412, 2541263, -, 852,12; cds-WP_000719962.1, NZ_CP025268.1, 2541368, 2541742, +, 375, 815; cds-WP_000745534.1, NZ_CP025268.1, 2541775, 2542509, +, 735, 35406; cds-WP _001336044.1, NZ_CP025268.1, 2542698, 2543609, -, 912, 1333; cds-WP_000021036.1, NZ_CP025268.1, 2543743, 2544840, -, 1098, 22; cds-WP _000852686.1, NZ_CP025268.1, 2544830, 2545705, -, 876, 351; cds-WP_000458406.1, NZ_CP025268.1, 2545705, 2546538, -, 834, 0; cds-WP _000290230.1, NZ_CP025268.1, 2546538, 2547554, -, 1017, 32; cds-WP_000517431.1, NZ_CP025268.1, 2547858, 2548649, -, 792, 6405; cds-WP_000966470.1, NZ_CP025268.1, 2548778, 2549635, -, 858, 15; cds-WP_001159160.1, NZ_CP025268.1, 2549799, 2550695, +, 897, 0; cds-WP_001040483.1, NZ_CP025268.1, 2550699, 2552123, +, 1425, 54231; cds-WP_001327042.1, NZ_CP025268.1, 2552128, 2553432, +, 1305, 140; cds-WP_001350538.1, NZ_CP025268.1, 2553672, 2554571, -, 900, 9; cds-WP _000838944.1, NZ_CP025268.1, 2554667, 2555242, -, 576, 16107; cds-WP _001350539.1, NZ_CP025268.1, 2555303, 2555752, -, 450, 678; cds-WP _000405996.1, NZ_CP025268.1, 2555739, 2556164, -, 426, 4521; cds-WP _000102886.1, NZ_CP025268.1, 2556378, 2557247, +, 870, 1091; cds-WP _000801365.1, NZ_CP025268.1, 2557251, 2558150, +, 900, 184859; cds-WP_000753690.1, NZ_CP025268.1, 2558156, 2559208, -, 1053, 0; cds-WP_000606257.1, NZ_CP025268.1, 2559254, 2559754, -, 501, 0; cds-WP _001111023.1, NZ_CP025268.1, 2559767, 2560426, -, 660, 0; cds-WP_000372316.1, NZ_CP025268.1, 2560436, 2561323, -, 888, 36890; cds-WP_000769961.1, NZ_CP025268.1, 2561344, 2562705, -, 1362, 12645; cds-WP_000047773.1, NZ_CP025268.1, 2562884, 2564092, +, 1209, 24217; cds-WP _000458337.1, NZ_CP025268.1, 2564283, 2564924, +, 642, 1887; cds-WP_010723122.1, NZ_CP025268.1, 2565394, 2565639, +, 246, 0; cds-WP _000683389.1, NZ_CP025268.1, 2565651, 2566019, +, 369, 0; cds-WP _000398186.1, NZ_CP025268.1, 2566137, 2566553, +, 417, 0; cds-WP _000052882.1, NZ_CP025268.1, 2566550, 2567143, +, 594, 0; cds-WP _000770705.1, NZ_CP025268.1, 2567618, 2567995, +, 378, 0; cds-WP_000865921.1, NZ_CP025268.1, 2568006, 2568398, +, 393, 0; cds-WP _010723125.1, NZ_CP025268.1, 2568549, 2569358, +, 810, 8; cds-WP_001097542.1, NZ_CP025268.1, 2569507, 2570910, -, 1404, 0; cds-WP_000512372.1, NZ_CP025268.1, 2570907, 2572133, -, 1227, 1; cds-WP _001326575.1, NZ_CP025268.1, 2572350, 2573537, -, 1188, 0; cds-WP _000929729.1, NZ_CP025268.1, 2573527, 2574363, -, 837, 0; cds-WP _001075716.1, NZ_CP025268.1, 2574374, 2575777, -, 1404, 0; cds-WP_000762196.1, NZ_CP025268.1, 2575789, 2576076, -, 288, 0; cds-WP _000387713.1, NZ_CP025268.1, 2576183, 2576476, -,
294, 0; cds-WP _000582977.1, NZ_CP025268.1, 2576515, 2577531, -, 1017, 0; cds-WP_000651298.1, NZ_CP025268.1, 2577528, 2578331, -, 804, 0; cds-WP_000733906.1, NZ_CP025268.1, 2578328, 2579029, -, 702, 0; cds-WP_000820763.1, NZ_CP025268.1, 2579004, 2579483, -, 480, 0; cds-WP_000356956.1, NZ_CP025268.1, 2579496, 2579831, -, 336, 0; cds-WP_000342644.1, NZ_CP025268.1, 2580124, 2582403, -, 2280, 231; cds-WP_001003709.1, NZ_CP025268.1, 2582692, 2583642, +, 951, 0; cds-WP _000087280.1, NZ_CP025268.1, 2583662, 2585665, +, 2004, 39200; cds-WP _001270542.1, NZ_CP025268.1, 2585760, 2586803, -, 1044, 2185; cds-WP _001300814.1, NZ_CP025268.1, 2586929, 2587504, -, 576, 2; cds-WP _001078880.1, NZ_CP025268.1, 2587572, 2589551, -, 1980, 0; cds-WP_001300881.1, NZ_CP025268.1, 2589757, 2591457, +, 1701, 16492; cds-WP_001273151.1, NZ_CP025268.1, 2591621, 2594734, +, 3114, 116306; cds-WP _001386977.1, NZ_CP025268.1, 2594833, 2594892, -, 60, 3003; cds-WP _000258236.1, NZ_CP025268.1, 2595273, 2595629, +, 357, 173; cds-WP _001277801.1, NZ_CP025268.1, 2595633, 2596760, +, 1128, 12471; cds-WP _000383836.1, NZ_CP025268.1, 2596788, 2596988, +, 201, 425; cds-WP_000679799.1, NZ_CP025268.1, 2597098, 2597796, -, 699, 10436; cds-WP_000829360.1, NZ_CP025268.1, 2597870, 2599885, -, 2016, 67780; cds-WP_001267498.1, NZ_CP025268.1, 2599900, 2600763, -, 864, 68787; cds-WP _001295467.1, NZ_CP025268.1, 2600931, 2601644, -, 714, 17026; cds-WP _001297320.1, NZ_CP025268.1, 2601857, 2602891, -, 1035, 289039; cds-WP_001311023.1, NZ_CP025268.1, 2602908, 2603786, -, 879, 382737; cds-WP_000176187.1, NZ_CP025268.1, 2603932, 2604504, +, 573, 29; cds-CXP41_RS13680, NZ_CP025268.1, 2604504, 2604975, +, 472, 246; cds-WP _001336048.1, NZ_CP025268.1, 2605228, 2605845, +, 618, 254; cds-WP_000339455.1, NZ_CP025268.1, 2605845, 2607863, +, 2019, 1; cds-WP_001311024.1, NZ_CP025268.1, 2607874, 2608821, +, 948, 0; cds-WP _000429104.1, NZ_CP025268.1, 2608838, 2610277, +, 1440, 0; cds-WP _000147987.1, NZ_CP025268.1, 2610289, 2610939, +, 651, 0; cds-WP _000122577.1, NZ_CP025268.1, 2610944, 2612524, +, 1581, 0; cds-WP_001102321.1, NZ_CP025268.1, 2612514, 2614181, +, 1668, 0; cds-WP_000916055.1, NZ_CP025268.1, 2614191, 2614736, +, 546, 0; cds-WP _000075558.1, NZ_CP025268.1, 2614733, 2615491, +, 759, 0; cds-WP_001326580.1, NZ_CP025268.1, 2615484, 2615897, +, 414, 0; cds-WP _001251544.1, NZ_CP025268.1, 2615927, 2617939, +, 2013, 47893; cds-WP_101348630.1, NZ_CP025268.1, 2617961, 2618806, +, 846, 42464; cds-WP_000892044.1, NZ_CP025268.1, 2618844, 2619905, -, 1062, 218445; cds-WP _000489667.1, NZ_CP025268.1, 2620118, 2621581, +, 1464, 89; cds-WP _000166446.1, NZ_CP025268.1, 2621602, 2621961, +, 360, 7; cds-WP _001307333.1, NZ_CP025268.1, 2622099, 2622800, -, 702, 66541; cds-WP _000198328.1, NZ_CP025268.1, 2622895, 2624184, -, 1290, 22452; cds-WP _001295473.1, NZ_CP025268.1, 2624270, 2624896, -, 627, 142515; cds-WP_001336050.1, NZ_CP025268.1, 2625221, 2626258, +, 1038, 32620; cds-WP _001028612.1, NZ_CP025268.1, 2626258, 2626896, +, 639, 25350; cds-WP _000529576.1, NZ_CP025268.1, 2627068, 2629134, +, 2067, 486415; cds-WP _001121363.1, NZ_CP025268.1, 2629139, 2630680, +, 1542, 399875; cds-WP _000772731.1, NZ_CP025268.1, 2630719, 2632962, -, 2244, 106861; cds-WP _000017552.1, NZ_CP025268.1, 2633144, 2633296, -, 153, 0; cds-WP_000076001.1, NZ_CP025268.1, 2633314, 2633505, +, 192, 149; cds-WP_001295476.1, NZ_CP025268.1, 2633816, 2634334, +, 519, 2; cds-WP_000755173.1, NZ_CP025268.1, 2634350, 2634889, +, 540, 219; cds-WP _000138270.1, NZ_CP025268.1, 2634982, 2636559, -, 1578, 1854312; cds-WP _001299507.1, NZ_CP025268.1, 2636628, 2638094, -, 1467, 633481; cds-WP _000937912.1, NZ_CP025268.1, 2638256, 2639626, +, 1371, 115550; cds-WP _001301158.1, NZ_CP025268.1, 2639623, 2639838, -, 216, 48464; cds-WP_101348631.1, NZ_CP025268.1, 2639908, 2641380, -, 1473, 681902; cds-WP_001177052.1, NZ_CP025268.1, 2641498, 2642676, -, 1179, 102626; cds-WP _000409205.1, NZ_CP025268.1, 2642687, 2643307, -, 621, 160236; cds-WP_001107167.1, NZ_CP025268.1, 2643325, 2644599, -, 1275, 1102527; cds-WP _000551807.1, NZ_CP025268.1, 2644710, 2645828, -, 1119, 139521; cds-WP _001090866.1, NZ_CP025268.1, 2645855, 2646868, -, 1014, 117721; cds-WP_000003317.1, NZ_CP025268.1, 2647153, 2648307, -, 1155, 23286; cds-WP _000963837.1, NZ_CP025268.1, 2648457, 2648888, -, 432, 24175; cds-WP_001137675.1, NZ_CP025268.1, 2649037, 2651349, -, 2313, 185542; cds-WP_101348632.1, NZ_CP025268.1, 2651350, 2656311, -, 4962, 70; cds-WP_000108626.1, NZ_CP025268.1, 2656518, 2657363, +, 846, 782; cds-WP _001295479.1, NZ_CP025268.1, 2658181, 2658957, -, 777, 89371; cds-WP_101348633.1, NZ_CP025268.1, 2659099, 2660382, -, 1284, 76601; cds-WP _000523616.1, NZ_CP025268.1, 2660560, 2660760, -, 201, 97472; cds-WP_001124469.1, NZ_CP025268.1, 2660772, 2661107, -, 336, 229194; cds-WP_001196613.1, NZ_CP025268.1, 2661109, 2662959, -, 1851, 107307; cds-WP _000384413.1, NZ_CP025268.1, 2662976, 2663491, -, 516, 574240; cds-WP_000028953.1, NZ_CP025268.1, 2663587, 2663910, -, 324, 141560; cds-WP_000331707.1, NZ_CP025268.1, 2663927, 2664313, -, 387, 57309; cds-WP _001295373.1, NZ_CP025268.1, 2664341, 2665555, -, 1215, 1576250; cds-WP_001241357.1, NZ_CP025268.1, 2665667, 2666155, -, 489, 46328; cds-WP_000940019.1, NZ_CP025268.1, 2666607, 2667347, -, 741, 117591; cds-WP_000553451.1, NZ_CP025268.1, 2667466, 2668269, +, 804, 376167; cds-WP_001297705.1, NZ_CP025268.1, 2668414, 2669268, +, 855, 241865; cds-WP_000982994.1, NZ_CP025268.1, 2669459, 2670739, +, 1281, 7; cds-WP_000244193.1, NZ_CP025268.1, 2670731, 2671870, -, 1140, 701; cds-WP _000423261.1, NZ_CP025268.1, 2672030, 2672920, -, 891, 0; cds-WP_000211172.1, NZ_CP025268.1, 2673056, 2674417, +, 1362, 0; cds-WP_001276072.1, NZ_CP025268.1, 2674414, 2674932, +, 519, 0; cds-WP_001080102.1, NZ_CP025268.1, 2674932, 2675252, +, 321, 0; cds-WP_001281379.1, NZ_CP025268.1, 2675249, 2676061, +, 813, 0; cds-WP _000660788.1, NZ_CP025268.1, 2676071, 2677273, +, 1203, 0; cds-WP_001094726.1, NZ_CP025268.1, 2677370, 2677792, +, 423, 0; cds-WP_000158547.1, NZ_CP025268.1, 2677840, 2678712, -, 873, 0; cds-WP_000700787.1, NZ_CP025268.1, 2678724, 2679818, -, 1095, 3; cds-WP _001276670.1, NZ_CP025268.1, 2679851, 2680849, -, 999, 0; cds-WP _000493470.1, NZ_CP025268.1, 2680874, 2682385, -, 1512, 0; cds-WP_001142427.1, NZ_CP025268.1, 2682408, 2683391, -, 984, 0; cds-WP_001333903.1, NZ_CP025268.1, 2683488, 2686769, -, 3282, 0; cds-WP_001200769.1, NZ_CP025268.1, 2686887, 2688080, +, 1194, 11; cds-WP _000919159.1, NZ_CP025268.1, 2688278, 2689531, -, 1254, 59463; cds-WP_101348634.1, NZ_CP025268.1, 2689859, 2691049, +, 1191, 1; cds-WP_000717694.1, NZ_CP025268.1, 2691094, 2691432, -, 339, 773; cds-WP_001295369.1, NZ_CP025268.1, 2691493, 2692827, -, 1335, 3523; cds-WP _001215861.1, NZ_CP025268.1, 2692817, 2693530, -, 714, 366; cds-WP _001311037.1, NZ_CP025268.1, 2693695, 2695122, -, 1428, 87240; cds-WP_000970102.1, NZ_CP025268.1, 2695680, 2699567, -, 3888, 498552; cds-WP_000734212.1, NZ_CP025268.1, 2699825, 2701381, +, 1557, 24121; cds-WP_001297409.1, NZ_CP025268.1, 2701378, 2701881, -, 504, 5; cds-WP _000190655.1, NZ_CP025268.1, 2701939, 2702574, -, 636, 0; cds-WP_001013779.1, NZ_CP025268.1, 2702783, 2703631, +, 849, 0; cds-WP_001196283.1, NZ_CP025268.1, 2703687, 2703947, +, 261, 17; cds-WP_000128776.1, NZ_CP025268.1, 2704141, 2704221, -, 81, 4590; cds-WP_000986023.1, NZ_CP025268.1, 2704642, 2705022, -, 381, 21681; cds-WP_001297412.1, NZ_CP025268.1, 2705022, 2705753, -, 732, 214727; cds-WP _000399404.1, NZ_CP025268.1, 2705765, 2706493, -, 729, 58386; cds-WP _000020749.1, NZ_CP025268.1, 2706505, 2707410, -, 906, 302898; cds-WP_001068343.1, NZ_CP025268.1, 2707407, 2708087, -, 681, 650608; cds-WP_000002541.1, NZ_CP025268.1, 2708359, 2709333, -, 975, 616421; cds-WP_101348635.1, NZ_CP025268.1, 2709349, 2711148, -, 1800, 636521; cds-WP_000589068.1, NZ_CP025268.1, 2711346, 2711825, -, 480, 308714; cds-WP_000812053.1, NZ_CP025268.1, 2711822, 2712778, -, 957, 1166867; cds-WP _001168459.1, NZ_CP025268.1, 2712778, 2713428, -, 651, 1182023; cds-WP_001295364.1, NZ_CP025268.1, 2713461, 2714036, -, 576, 218293; cds-WP_001303621.1, NZ_CP025268.1, 2714033, 2714188, -, 156, 3; cds-WP _001094491.1, NZ_CP025268.1, 2714444, 2716066, +, 1623, 0; cds-WP _001295363.1, NZ_CP025268.1, 2716051, 2716788, -, 738, 247; cds-WP_101348636.1, NZ_CP025268.1, 2716920, 2718254, +, 1335, 81542; cds-WP_001300638.1, NZ_CP025268.1, 2718463, 2719344, -, 882, 13084; cds-WP _000189207.1, NZ_CP025268.1, 2719447, 2720034, +, 588, 0; cds-WP_000627807.1, NZ_CP025268.1, 2720090, 2720473, -, 384, 0; cds-WP_001262716.1, NZ_CP025268.1, 2720778, 2721467, +, 690,127; cds-WP_000997403.1, NZ_CP025268.1, 2721515, 2722552, -, 1038, 187613; cds-WP_001098726.1, NZ_CP025268.1, 2722759, 2723178, +, 420, 4; cds-WP _001300438.1, NZ_CP025268.1, 2723247, 2723945, +, 699, 7; cds-WP_000083005.1, NZ_CP025268.1, 2723977, 2726637, +, 2661, 40731; cds-WP_211180504.1, NZ_CP025268.1, 2726726, 2726767, +, 42, 3915; cds-WP_000949265.1, NZ_CP025268.1, 2726751, 2728106, +, 1356, 321450; cds-WP_001300818.1, NZ_CP025268.1, 2728152, 2728475, +, 324, 410892; cds-WP_000841103.1, NZ_CP025268.1, 2728472, 2729770, -, 1299, 1009147; cds-WP_001235102.1, NZ_CP025268.1, 2735624, 2738197, -, 2574, 568703; cds-WP _000040169.1, NZ_CP025268.1, 2738327, 2739058, -, 732, 55341; cds-WP _000079100.1, NZ_CP025268.1, 2739055, 2740035, -, 981, 212914; cds-WP_000197686.1, NZ_CP025268.1, 2740170, 2740907, +, 738, 166147; cds-WP_000178456.1, NZ_CP025268.1, 2741178, 2741519, +, 342, 154602; cds-WP_010723158.1, NZ_CP025268.1, 2741623, 2741670, +, 48, 25784; cds-WP_000200120.1, NZ_CP025268.1, 2741769, 2742929, +, 1161, 100613; cds-WP_000225229.1, NZ_CP025268.1, 2742972, 2744093, -, 1122, 7501; cds-WP_001168037.1, NZ_CP025268.1, 2744104, 2745174, -, 1071, 70; cds-WP_000976004.1, NZ_CP025268.1, 2745384, 2745749, +, 366, 0; cds-WP_001212391.1, NZ_CP025268.1, 2745899, 2746417, +, 519, 42; cds-WP_097519179.1, NZ_CP025268.1, 2746407, 2747633, +, 1227, 3853; cds-WP_000589847.1, NZ_CP025268.1, 2747649, 2748131, +, 483, 4254; cds-WP_000065253.1, NZ_CP025268.1, 2748207, 2748554, -, 348, 2311327; cds-WP_000264777.1, NZ_CP025268.1, 2748596, 2749363, -, 768, 6212154; cds-WP_000043335.1, NZ_CP025268.1, 2749394, 2749942, -, 549, 3145360; cds-WP_000256450.1, NZ_CP025268.1, 2749961, 2750209, -, 249, 407517; cds-WP_000460035.1, NZ_CP025268.1, 2750458, 2751819, -, 1362, 340057; cds-WP_001338897.1, NZ_CP025268.1, 2751986, 2752777, +, 792, 81934; cds-WP_010723175.1, NZ_CP025268.1, 2752798, 2754084, +, 1287, 15042; cds-WP_001393454.1, NZ_CP025268.1, 2754139, 2754732, -, 594, 184005; cds-WP_001059169.1, NZ_CP025268.1, 2754855, 2755733, +, 879, 281953; cds-WP _000880910.1, NZ_CP025268.1, 2755819, 2757480, +, 1662, 62592; cds-WP_001203437.1, NZ_CP025268.1, 2757629, 2757970, +, 342, 1832; cds-WP_001117838.1, NZ_CP025268.1, 2758032, 2758322, -, 291, 2; cds-WP _001300750.1, NZ_CP025268.1, 2758312, 2758749, -, 438, 62; cds-WP_000162574.1, NZ_CP025268.1, 2758920, 2759402, +, 483, 5962; cds-WP_000101723.1, NZ_CP025268.1, 2760183, 2761424, +, 1242, 18732; cds-WP_000510827.1, NZ_CP025268.1, 2761668, 2762624, -, 957, 0; cds-WP_001065343.1, NZ_CP025268.1, 2762668, 2762880, +, 213, 2; cds-WP_000481742.1, NZ_CP025268.1, 2763009, 2764418, +, 1410, 0; cds-WP_000220524.1, NZ_CP025268.1, 2764571, 2765197, +, 627, 0; cds-WP_000136402.1, NZ_CP025268.1, 2765375, 2767564, -, 2190, 666; cds-WP_000445380.1, NZ_CP025268.1, 2767561, 2769177, -, 1617, 24098; cds-WP_000502050.1, NZ_CP025268.1, 2769537, 2769800, -, 264, 0; cds-WP_000155570.1, NZ_CP025268.1, 2769941, 2771014, +, 1074, 185682; cds-WP _000461704.1, NZ_CP025268.1, 2771007, 2771378, +, 372, 85742; cds-WP_000787597.1, NZ_CP025268.1, 2771734, 2772597, +, 864, 2146; cds-WP _000197391.1, NZ_CP025268.1, 2772689, 2773510, +, 822, 0; cds-WP _000189409.1, NZ_CP025268.1, 2773726, 2774427, +, 702, 0; cds-WP _001144029.1, NZ_CP025268.1, 2774468, 2774704, +, 237, 0; cds-WP_000824222.1, NZ_CP025268.1, 2774704, 2775147, +, 444, 0; cds-WP_000702495.1, NZ_CP025268.1, 2775171, 2775638, +, 468, 0; cds-WP _000065803.1, NZ_CP025268.1, 2775863, 2776177, -, 315, 0; cds-CXP41_RS14535, NZ_CP025268.1, 2776190, 2777199, -, 1010, 1454; cds-WP_000896263.1, NZ_CP025268.1, 2777341, 2779044, +, 1704, 0; cds-CXP41_RS14545, NZ_CP025268.1, 2779616, 2779839, +, 224, 0; cds-WP_000211841.1, NZ_CP025268.1, 2779942, 2780400, +, 459, 0; cds-WP_001407480.1, NZ_CP025268.1,
2780409, 2780891, +, 483, 0; cds-WP_001395452.1, NZ_CP025268.1, 2780900, 2781100, +, 201, 5; cds-WP_000072690.1, NZ_CP025268.1, 2781138, 2781455, +, 318, 0; cds-WP_001094400.1, NZ_CP025268.1, 2781476, 2781805, +, 330, 0; cds-CXP41_RS14575, NZ_CP025268.1, 2782169, 2786748, -, 4580, 5; cds-WP_001120794.1, NZ_CP025268.1, 2786813, 2786932, -, 120, 0; cds-WP_071524906.1, NZ_CP025268.1, 2787087, 2787305, -, 219, 1657; cds-CXP41_RS24250, NZ_CP025268.1, 2787660, 2789033, -, 1374, 0; cds-CXP41_RS24390, NZ_CP025268.1, 2790419, 2792670, +, 2252, 0; cds-WP_000993126.1, NZ_CP025268.1, 2793006, 2793983, +, 978, 0; cds-WP_000271909.1, NZ_CP025268.1, 2794003, 2795271, +, 1269, 0; cds-WP_000772831.1, NZ_CP025268.1, 2795294, 2796742, +, 1449, 0; cds-WP_001087611.1, NZ_CP025268.1, 2796756, 2798036, +, 1281, 2316; cds-WP_001295173.1, NZ_CP025268.1, 2798274, 2799674, +, 1401, 1; cds-WP_077392725.1, NZ_CP025268.1, 2799695, 2800357, +, 663, 0; cds-WP_000522415.1, NZ_CP025268.1, 2800358, 2800807, -, 450, 0; cds-WP_101348637.1, NZ_CP025268.1, 2800891, 2801049, -, 159, 153; cds-WP_000137280.1, NZ_CP025268.1, 2801232, 2801531, +, 300, 0; cds-WP_001229442.1, NZ_CP025268.1, 2801541, 2802065, +, 525, 1; cds-WP_000115383.1, NZ_CP025268.1, 2802112, 2802516, -, 405, 6; cds-WP_000492656.1, NZ_CP025268.1, 2803185, 2803634, +, 450, 0; cds-WP_000281320.1, NZ_CP025268.1, 2803671, 2804015, -, 345, 0; cds-WP_001295174.1, NZ_CP025268.1, 2804167, 2804496, +, 330, 0; cds-WP_001223227.1, NZ_CP025268.1, 2804744, 2804989, +, 246, 24; cds-WP_000080947.1, NZ_CP025268.1, 2804986, 2805396, +, 411, 0; cds-WP_000246534.1, NZ_CP025268.1, 2805369, 2807513, +, 2145, 5527; cds-WP_000777969.1, NZ_CP025268.1, 2807523, 2808482, +, 960, 60517; cds-WP_000985494.1, NZ_CP025268.1, 2808836, 2810038, +, 1203, 1219492; cds-WP_000774988.1, NZ_CP025268.1, 2810031, 2811095, +, 1065, 496755; cds-WP_001216525.1, NZ_CP025268.1, 2811153, 2812145, +, 993, 371508; cds-CXP41_RS14730, NZ_CP025268.1, 2812337, 2813514, +, 1178, 45693; cds-WP_000445651.1, NZ_CP025268.1, 2813638, 2814375, +, 738, 83038; cds-WP_000119763.1, NZ_CP025268.1, 2814365, 2814700, +, 336, 4170; cds-WP_000378442.1, NZ_CP025268.1, 2814791, 2815321, +, 531, 67664; cds-WP_001326681.1, NZ_CP025268.1, 2815448, 2816620, +, 1173, 470; cds-WP_001295176.1, NZ_CP025268.1, 2816637, 2818175, +, 1539, 560146; cds-WP_001130211.1, NZ_CP025268.1, 2818239, 2818754, -, 516, 1545; cds-WP_000611804.1, NZ_CP025268.1, 2818904, 2820460, -, 1557, 178897; cds-WP_001287454.1, NZ_CP025268.1, 2820533, 2820961, -, 429, 53022; cds-WP_000273290.1, NZ_CP025268.1, 2820958, 2821524, -, 567, 7998; cds-WP_000906486.1, NZ_CP025268.1, 2822982, 2823167, -, 186, 76649; cds-WP_000047184.1, NZ_CP025268.1, 2823402, 2826032, -, 2631, 941092; cds-WP_000140508.1, NZ_CP025268.1, 2826160, 2826660, -, 501, 0; cds-WP_000963143.1, NZ_CP025268.1, 2826729, 2827790, -, 1062, 816627; cds-WP_000132231.1, NZ_CP025268.1, 2827870, 2828367, -, 498, 814; cds-CXP41_RS14830, NZ_CP025268.1, 2828517, 2829596, -, 1080, 104118; cds-WP_000573321.1, NZ_CP025268.1, 2829852, 2830415, +, 564, 0; cds-WP_000148878.1, NZ_CP025268.1, 2830412, 2831371, +, 960, 0; cds-WP_000216194.1, NZ_CP025268.1, 2831382, 2831753, +, 372, 270; cds-WP_001077358.1, NZ_CP025268.1, 2831757, 2832536, +, 780, 0; cds-WP_000252908.1, NZ_CP025268.1, 2832641, 2833000, +, 360, 0; cds-WP_000804550.1, NZ_CP025268.1, 2833067, 2833840, +, 774, 26; cds-WP_001287420.1, NZ_CP025268.1, 2833833, 2834798, +, 966, 6; cds-WP_000010749.1, NZ_CP025268.1, 2834795, 2836309, -, 1515, 72440; cds-WP_000029634.1, NZ_CP025268.1, 2836496, 2837935, +, 1440, 9763; cds-WP_000064752.1, NZ_CP025268.1, 2837932, 2839065, +, 1134, 12052; cds-WP_001107704.1, NZ_CP025268.1, 2839193, 2841445, -, 2253, 28; cds-WP_001078777.1, NZ_CP025268.1, 2841598, 2842125, -, 528, 0; cds-WP_001394680.1, NZ_CP025268.1, 2842274, 2843284, -, 1011, 2; cds-WP_001107828.1, NZ_CP025268.1, 2843544, 2845001, +, 1458, 0; cds-WP_000110363.1, NZ_CP025268.1, 2845010, 2846434, +, 1425, 146496; cds-WP_000132961.1, NZ_CP025268.1, 2846593, 2847063, -, 471,14; cds-WP_001291921.1, NZ_CP025268.1, 2847056, 2847466, -, 411, 0; cds-WP_000067392.1, NZ_CP025268.1, 2847463, 2848230, -, 768, 0; cds-WP_000493781.1, NZ_CP025268.1, 2848230, 2848772, -, 543, 0; cds-WP_001288122.1, NZ_CP025268.1, 2848782, 2850491, -, 1710, 6; cds-WP_000115224.1, NZ_CP025268.1, 2850509, 2851432, -, 924, 0; cds-WP_001274396.1, NZ_CP025268.1, 2851435, 2853261, -, 1827, 0; cds-WP_001079186.1, NZ_CP025268.1, 2853258, 2853869, -, 612, 0; cds-WP_000158056.1, NZ_CP025268.1, 2853994, 2854455, -, 462, 0; cds-WP_001299100.1, NZ_CP025268.1, 2854667, 2855017, +, 351, 0; cds-WP_000337665.1, NZ_CP025268.1, 2855021, 2855893, +, 873, 0; cds-WP_071342902.1, NZ_CP025268.1, 2855884, 2856156, +, 273, 44; cds-WP_001212985.1, NZ_CP025268.1, 2856156, 2857277, +, 1122, 0; cds-WP_001059922.1, NZ_CP025268.1, 2857274, 2858284, +, 1011, 0; cds-CXP41_RS14980, NZ_CP025268.1, 2858358, 2860435, +, 2078, 87; cds-WP_000015497.1, NZ_CP025268.1, 2860472, 2860825, -, 354, 4; cds-WP_000611932.1, NZ_CP025268.1, 2860902, 2861036, -, 135, 0; cds-WP_101348638.1, NZ_CP025268.1, 2861226, 2861600, +, 375, 0; cds-WP_001141337.1, NZ_CP025268.1, 2862069, 2862725, +, 657, 6700; cds-WP_001300386.1, NZ_CP025268.1, 2862775, 2863542, -, 768, 0; cds-WP_000848004.1, NZ_CP025268.1, 2863738, 2864646, +, 909, 0; cds-WP_001393459.1, NZ_CP025268.1, 2864643, 2865809, +, 1167, 0; cds-WP_001278994.1, NZ_CP025268.1, 2865901, 2866539, +, 639, 0; cds-WP_001136934.1, NZ_CP025268.1, 2866544, 2867320, +, 777, 0; cds-WP_000104441.1, NZ_CP025268.1, 2867409, 2868773, +, 1365, 5754; cds-WP_000081588.1, NZ_CP025268.1, 2868867, 2869859, -, 993, 17399; cds-WP_001272592.1, NZ_CP025268.1, 2869922, 2871061, -, 1140, 232423; cds-WP_000254708.1, NZ_CP025268.1, 2871201, 2871827, -, 627, 200532; cds-WP_001295182.1, NZ_CP025268.1, 2871821, 2872582, -, 762, 318959; cds-WP_000568943.1, NZ_CP025268.1, 2872563, 2873612, -, 1050, 9641; cds-WP_001219242.1, NZ_CP025268.1, 2873609, 2874088, -, 480, 536649; cds-WP_000246138.1, NZ_CP025268.1, 2874088, 2874798, -, 711, 620891; cds-WP_000517476.1, NZ_CP025268.1, 2874817, 2875128, -, 312, 149070; cds-WP_001246104.1, NZ_CP025268.1, 2875322, 2875645, -, 324, 5813; cds-WP_001173673.1, NZ_CP025268.1, 2875695, 2876300, -, 606, 2123; cds-WP_001090361.1, NZ_CP025268.1, 2876300, 2877727, -, 1428,9; cds-WP_000372108.1, NZ_CP025268.1, 2877729, 2878637, -, 909, 2; cds-WP_101348639.1, NZ_CP025268.1, 2878889, 2879926, +, 1038, 47120; cds-WP_001381369.1, NZ_CP025268.1, 2880877, 2881161, -, 285, 0; cds-WP_000220066.1, NZ_CP025268.1, 2881163, 2882080, -, 918, 0; cds-WP_000281400.1, NZ_CP025268.1, 2882096, 2882695, -, 600, 0; cds-WP_001334996.1, NZ_CP025268.1, 2882682, 2883356, -, 675, 0; cds-WP_000064450.1, NZ_CP025268.1, 2883359, 2884450, -, 1092, 4; cds-WP_000752800.1, NZ_CP025268.1, 2884463, 2884945, -, 483, 0; cds-WP_001050401.1, NZ_CP025268.1, 2884938, 2886446, -, 1509, 0; cds-WP_000433152.1, NZ_CP025268.1, 2886861, 2889527, -, 2667, 2; cds-WP_000039850.1, NZ_CP025268.1, 2889886, 2890620, -, 735, 5; cds-WP_001290679.1, NZ_CP025268.1, 2890695, 2892407, -, 1713, 0; cds-WP_000211913.1, NZ_CP025268.1, 2892407, 2894206, -, 1800, 21; cds-WP_000108294.1, NZ_CP025268.1, 2894525, 2894887, +, 363, 47464; cds-WP_001301334.1, NZ_CP025268.1, 2894965, 2896236, +, 1272, 165482; cds-WP_101348640.1, NZ_CP025268.1, 2896227, 2896484, +, 258, 51629; cds-WP_001130266.1, NZ_CP025268.1, 2896501, 2897076, +, 576, 50489; cds-WP_001299097.1, NZ_CP025268.1, 2897224, 2898084, -, 861, 6; cds-WP_001299652.1, NZ_CP025268.1, 2898081, 2898860, -, 780, 0; cds-WP_000147666.1, NZ_CP025268.1, 2898838, 2900247, -, 1410, 0; cds-WP_000059307.1, NZ_CP025268.1, 2900269, 2901723, -, 1455, 0; cds-WP_000021334.1, NZ_CP025268.1, 2901793, 2902578, -, 786, 0; cds-WP_001164544.1, NZ_CP025268.1, 2902897, 2904174, +, 1278, 0; cds-WP_000039688.1, NZ_CP025268.1, 2904201, 2905679, +, 1479, 0; cds-WP_001199973.1, NZ_CP025268.1, 2907052, 2907723, -, 672, 43919; cds-WP_001288228.1, NZ_CP025268.1, 2907862, 2908002, +, 141, 0; cds-WP_001268460.1, NZ_CP025268.1, 2908016, 2908888, +, 873, 2; cds-WP_000036723.1, NZ_CP025268.1, 2908948, 2910246, -, 1299, 1828361; cds-WP_000210878.1, NZ_CP025268.1, 2910334, 2911971, -, 1638, 918782; cds-WP_001071648.1, NZ_CP025268.1, 2912199, 2912990, -, 792, 56644; cds-WP_000254738.1, NZ_CP025268.1, 2913061, 2913396, -, 336, 60670; cds-WP_000581937.1, NZ_CP025268.1, 2913396, 2913644, -, 249, 203672; cds-WP_000226815.1, NZ_CP025268.1, 2913722, 2915956, -, 2235, 525863; cds-WP_000046812.1, NZ_CP025268.1, 2916004, 2917305, -, 1302, 7; cds-WP_101348641.1, NZ_CP025268.1, 2917362, 2920118, +, 2757, 251862; cds-WP_000098255.1, NZ_CP025268.1, 2920350, 2921690, -, 1341, 0; cds-WP_000235391.1, NZ_CP025268.1, 2921711, 2923051, -, 1341, 1; cds-WP_000097072.1, NZ_CP025268.1, 2923053, 2924405, -, 1353, 0; cds-WP_000807753.1, NZ_CP025268.1, 2924840, 2925289, -, 450, 77; cds-WP_000890001.1, NZ_CP025268.1, 2925307, 2926089, -, 783, 62; cds-WP_000206990.1, NZ_CP025268.1, 2926089, 2926418, -, 330, 70553; cds-WP_000342431.1, NZ_CP025268.1, 2927040, 2927585, -, 546, 9671; cds-WP_000100421.1, NZ_CP025268.1, 2927653, 2928501, +, 849, 2129; cds-WP_000627995.1, NZ_CP025268.1, 2928613, 2929977, +, 1365, 114465; cds-WP_000450476.1, NZ_CP025268.1, 2930534, 2931823, +, 1290, 1184005; cds-WP_000626422.1, NZ_CP025268.1, 2931881, 2933248, +, 1368, 519042; cds-WP_000268232.1, NZ_CP025268.1, 2933360, 2934115, +, 756, 12278; cds-WP_000013588.1, NZ_CP025268.1, 2934170, 2935318, -, 1149, 16063; cds-WP_000440781.1, NZ_CP025268.1, 2935346, 2935993, -, 648, 0; cds-WP_000528603.1, NZ_CP025268.1, 2936540, 2937856, +, 1317, 0; cds-WP_101348642.1, NZ_CP025268.1, 2937889, 2939664, +, 1776, 1139; cds-WP_101348643.1, NZ_CP025268.1, 2939773, 2941191, +, 1419, 0; cds-WP_000920840.1, NZ_CP025268.1, 2941193, 2941615, +, 423, 3; cds-WP_000642344.1, NZ_CP025268.1, 2941673, 2942404, +, 732, 1375; cds-WP_001045520.1, NZ_CP025268.1, 2942448, 2943548, -, 1101, 62756; cds-WP_000203905.1, NZ_CP025268.1, 2943541, 2943936, -, 396, 14224; cds-WP_000044401.1, NZ_CP025268.1, 2943955, 2944872, -, 918, 23085; cds-WP_000750398.1, NZ_CP025268.1, 2945223, 2945450, -, 228, 2178; cds-WP_001300698.1, NZ_CP025268.1, 2945642, 2946847, +, 1206, 25385; cds-WP_000184261.1, NZ_CP025268.1, 2946847, 2947290, +, 444, 29069; cds-WP_000117728.1, NZ_CP025268.1, 2947341, 2948147, -, 807, 738; cds-WP_000678646.1, NZ_CP025268.1, 2948386, 2949483, -, 1098, 225922; cds-WP_000016907.1, NZ_CP025268.1, 2950062, 2951315, -, 1254, 278483; cds-WP_000237947.1, NZ_CP025268.1, 2951547, 2952878, +, 1332, 50066; cds-WP_000775955.1, NZ_CP025268.1, 2952940, 2954766, -, 1827, 77459; cds-WP_001285993.1, NZ_CP025268.1, 2954766, 2958308, -, 3543, 349202; cds-WP_001138201.1, NZ_CP025268.1, 2958301, 2961189, -, 2889, 923499; cds-WP_000946938.1, NZ_CP025268.1, 2961365, 2964733, -, 3369, 73873; cds-WP_001276460.1, NZ_CP025268.1, 2964746, 2965069, -, 324, 3; cds-WP_001078376.1, NZ_CP025268.1, 2965054, 2965461, -, 408, 0; cds-WP_001144315.1, NZ_CP025268.1, 2965458, 2966021, -, 564, 0; cds-WP_000857051.1, NZ_CP025268.1, 2966012, 2966482, -, 471, 0; cds-WP_000816232.1, NZ_CP025268.1, 2966666, 2967460, -, 795, 207006; cds-WP_000204658.1, NZ_CP025268.1, 2967467, 2968342, -, 876, 61363; cds-WP_000957910.1, NZ_CP025268.1, 2968493, 2970739, -, 2247, 454341; cds-WP_000564489.1, NZ_CP025268.1, 2970752, 2971282, -, 531, 17362; cds-WP 000082201.1, NZ_CP025268.1, 2971967, 2972656, +, 690, 35; cds-WP_000895624.1, NZ_CP025268.1, 2972725, 2973438, +, 714, 49574; cds-WP_000758655.1, NZ_CP025268.1, 2973576, 2973794, +, 219, 68449; cds-WP_001199295.1, NZ_CP025268.1, 2973902, 2974942, +, 1041, 10; cds-WP_000004616.1, NZ_CP025268.1, 2974974, 2976167, -, 1194, 4420; cds-WP_000899054.1, NZ_CP025268.1, 2976160, 2978319, -, 2160, 55420; cds-WP_000201063.1, NZ_CP025268.1, 2978904, 2979935, +, 1032, 54; cds-WP_001120711.1, NZ_CP025268.1, 2979942, 2981204, -, 1263, 48528; cds-WP_101348644.1, NZ_CP025268.1, 2981326, 2982261, +, 936, 0; cds-WP_000848659.1, NZ_CP025268.1, 2982248, 2982940, -, 693, 2546; cds-WP_000256438.1, NZ_CP025268.1, 2983069, 2984487, -, 1419, 204; cds-WP_000603502.1, NZ_CP025268.1, 2984802, 2985563, -, 762, 0; cds-WP_101348645.1, NZ_CP025268.1, 2985593, 2986429, -, 837, 0; cds-WP_000656030.1, NZ_CP025268.1, 2986716, 2987897, -, 1182, 90; cds-WP_000065950.1, NZ_CP025268.1, 2988152, 2989381, +, 1230, 61266;
cds-WP _001336169.1, NZ_CP025268.1, 2989841, 2990473, +, 633, 372; cds-WP_001300710.1, NZ_CP025268.1, 2990807, 2991616, +, 810, 31301; cds-WP _000568339.1, NZ_CP025268.1, 2991609, 2992091, +, 483, 0; cds-WP_000655820.1, NZ_CP025268.1, 2992124, 2992204, -, 81, 0; cds-WP_000350612.1, NZ_CP025268.1, 2992240, 2992665, -, 426, 0; cds-WP _001333802.1, NZ_CP025268.1, 2992859, 2993305, +, 447, 0; cds-WP_000102780.1, NZ_CP025268.1, 2993573, 2994064, +, 492, 0; cds-WP _000362702.1, NZ_CP025268.1, 2994399, 2995775, +, 1377, 0; cds-WP _000184091.1, NZ_CP025268.1, 2995943, 2996161, +, 219, 85; cds-WP _0013361671, NZ_CP025268.1, 2996244, 2996720, +, 477, 0; cds-CXP41_RS15625, NZ_CP025268.1, 2996765, 2997397, -, 633, 0; cds-WP _000606430.1, NZ_CP025268.1, 2997620, 2998051, -, 432, 0; cds-WP _001372633.1, NZ_CP025268.1, 2998054, 2998275, -, 222, 0; cds-CXP41_RS15645, NZ_CP025268.1, 2998268, 2998666, -, 399, 1562; cds-WP_088895425.1-4, NZ_CP025268.1;NZ_CP025268.1, 2998678;2999591, 2999591;2999906, -;-, 1229, 8180; cds-CXP41_RS15660, NZ_CP025268.1, 2999998, 3001134, -, 1137, 207; cds-WP _001301085.1, NZ_CP025268.1, 3001442, 3002197, -, 756, 0; cds-WP _000388150.1, NZ_CP025268.1, 3002612, 3004909, +, 2298, 1082; cds-WP_000459182.1, NZ_CP025268.1, 3004920, 3005798, +, 879, 0; cds-WP_001016605.1, NZ_CP025268.1, 3005795, 3006274, +, 480, 0; cds-WP_000417791.1, NZ_CP025268.1, 3006314, 3008092, -, 1779, 1; cds-WP _000859787.1, NZ_CP025268.1, 3008571, 3009758, +, 1188, 27; cds-WP _000110493.1, NZ_CP025268.1, 3009816, 3011012, +, 1197, 0; cds-WP_001107125.1, NZ_CP025268.1, 3011070, 3012281, +, 1212, 29; cds-WP_001264442.1, NZ_CP025268.1, 3012334, 3013719, +, 1386, 0; cds-WP_000037992.1, NZ_CP025268.1, 3013767, 3014699, +, 933, 0; cds-WP _001020381.1, NZ_CP025268.1, 3014919, 3016544, -, 1626, 0; cds-WP _001298920.1, NZ_CP025268.1, 3016591, 3017361, -, 771, 0; cds-WP_001272828.1, NZ_CP025268.1, 3017464, 3018042, +, 579, 0; cds-WP_000502395.1, NZ_CP025268.1, 3018364, 3021462, +, 3099, 0; cds-WP_000906252.1, NZ_CP025268.1, 3021465, 3022793, +, 1329, 0; cds-WP _000572462.1, NZ_CP025268.1, 3022844, 3023623, +, 780, 0; cds-WP_000583615.1, NZ_CP025268.1, 3023620, 3026490, +, 2871, 0; cds-WP_001280192.1, NZ_CP025268.1, 3026655, 3028055, +, 1401, 0; cds-WP _001336279.1, NZ_CP025268.1, 3028073, 3029389, +, 1317, 0; cds-WP _000012163.1, NZ_CP025268.1, 3029425, 3030792, +, 1368, 0; cds-WP _000838428.1, NZ_CP025268.1, 3030828, 3031316, -, 489, 22; cds-WP _001350545.1, NZ_CP025268.1, 3031316, 3033235, -, 1920, 0; cds-WP _001295374.1, NZ_CP025268.1, 3033671, 3035119, +, 1449, 49263; cds-WP_001010156.1, NZ_CP025268.1, 3035121, 3035246, +, 126, 0; cds-WP_120795390.1, NZ_CP025268.1, 3035243, 3035314, -, 72, 0; cds-WP _001192820.1, NZ_CP025268.1, 3035369, 3035917, +, 549, 17487; cds-WP _000003071.1, NZ_CP025268.1, 3035961, 3037478, -, 1518, 1378272; cds-WP _001701073.1, NZ_CP025268.1;NZ_CP025268.1, 3037488;3038512, 3038510;3038586, -;-, 1098, 1145535; cds-WP _000813200.1, NZ_CP025268.1, 3038677, 3040410, -, 1734, 216382; cds-WP_000715214.1, NZ_CP025268.1, 3040416, 3041126, -, 711, 93230; cds-WP _000806638.1, NZ_CP025268.1, 3041151, 3042047, -, 897, 11116; cds-WP _001055874.1, NZ_CP025268.1, 3042159, 3042680, +, 522, 54292; cds-WP _000244777.1, NZ_CP025268.1, 3042720, 3043127, -, 408, 71956; cds-WP _000354046.1, NZ_CP025268.1, 3043108, 3043374, -, 267, 101408; cds-WP _000886062.1, NZ_CP025268.1, 3043617, 3044597, +, 981, 28026; cds-WP _000250274.1, NZ_CP025268.1, 3044793, 3045452, -, 660, 94319; cds-WP_001182957.1, NZ_CP025268.1, 3045616, 3045927, -, 312, 3505; cds-WP _001336277.1, NZ_CP025268.1, 3045972, 3047405, +, 1434, 94899; cds-WP _000951964.1, NZ_CP025268.1, 3047462, 3048205, -, 744, 93; cds-WP _000195062.1, NZ_CP025268.1, 3048472, 3051345, -, 2874, 126198; cds-WP_001295377.1, NZ_CP025268.1, 3051464, 3051853, -, 390, 2; cds-WP _000068701.1, NZ_CP025268.1, 3051877, 3052971, -, 1095, 53; cds-WP_120795391.1, NZ_CP025268.1, 3053262, 3053345, +, 84, 0; cds-WP_001192229.1, NZ_CP025268.1, 3053419, 3054621, -, 1203, 611890; cds-WP _000111195.1, NZ_CP025268.1, 3054644, 3055822, -, 1179, 313724; cds-WP _001290136.1, NZ_CP025268.1, 3055819, 3057144, -, 1326, 87198; cds-WP_001295378.1, NZ_CP025268.1, 3057170, 3057748, -, 579, 221023; cds-WP _001276008.1, NZ_CP025268.1, 3057916, 3058245, +, 330, 348186; cds-WP_000192748.1, NZ_CP025268.1, 3058491, 3059093, +, 603, 200842; cds-WP _010723220.1, NZ_CP025268.1, 3059194, 3059253, -, 60, 0; cds-WP_001151604.1, NZ_CP025268.1, 3059482, 3060714, -, 1233, 32; cds-WP _000189743.1, NZ_CP025268.1, 3060970, 3061629, -, 660, 16; cds-WP_000545308.1, NZ_CP025268.1, 3061685, 3061915, -, 231, 0; cds-WP_000828351.1, NZ_CP025268.1, 3062057, 3062950, +, 894, 21890; cds-WP_101348647.1, NZ_CP025268.1, 3063154, 3065298, +, 2145, 0; cds-WP_000606525.1, NZ_CP025268.1, 3065291, 3066286, +, 996, 0; cds-WP_000122080.1, NZ_CP025268.1, 3066297, 3067082, +, 786, 0; cds-WP 000449965.1, NZ_CP025268.1, 3067106, 3068584, +, 1479, 26; cds-WP _000350807.1, NZ_CP025268.1, 3068581, 3069477, -, 897, 1; cds-WP_000669834.1, NZ_CP025268.1, 3069644, 3070384, -, 741, 34709; cds-WP_000493352.1, NZ_CP025268.1, 3070477, 3071112, -, 636, 4; cds-WP _000389818.1, NZ_CP025268.1, 3071251, 3072111, -, 861, 2849; cds-WP _000034372.1, NZ_CP025268.1, 3072469, 3073548, -, 1080, 2054720; cds-WP _000111269.1, NZ_CP025268.1, 3073763, 3074926, -, 1164, 1574310; cds-WP _000218480.1, NZ_CP025268.1, 3074976, 3075995, -, 1020, 63675; cds-WP _000687705.1, NZ_CP025268.1, 3076280, 3076993, -, 714, 1901; cds-WP _000224147.1, NZ_CP025268.1, 3076990, 3077499, -, 510, 24562; cds-WP_000987283.1, NZ_CP025268.1, 3077521, 3078486, -, 966, 0; cds-WP_000853247.1, NZ_CP025268.1, 3078483, 3079760, -, 1278, 7; cds-WP_101348648.1, NZ_CP025268.1, 3079775, 3081163, -, 1389, 0; cds-WP_001239650.1, NZ_CP025268.1, 3081191, 3081634, -, 444, 0; cds-WP _000098614.1, NZ_CP025268.1, 3081948, 3083939, -, 1992, 1740431; cds-WP _001326497.1, NZ_CP025268.1, 3084217, 3084975, +, 759, 171; cds-WP_101348649.1, NZ_CP025268.1, 3085181, 3086101, -, 921, 229307; cds-WP_101348650.1, NZ_CP025268.1, 3086239, 3088215, -, 1977, 237048; cds-WP _001297406.1, NZ_CP025268.1, 3088224, 3088355, -, 132, 23014; cds-WP _001303650.1, NZ_CP025268.1, 3088491, 3088706, +, 216, 14109; cds-WP_001062128.1, NZ_CP025268.1, 3089010, 3090164, +, 1155, 464510; cds-WP_001112301.1, NZ_CP025268.1, 3090588, 3091982, +, 1395, 1; cds-WP_001300769.1, NZ_CP025268.1, 3092059, 3092556, +, 498, 167; cds-WP _000286500.1, NZ_CP025268.1, 3092651, 3093358, +, 708, 6672; cds-WP_001222509.1, NZ_CP025268.1, 3093438, 3094169, +, 732, 51; cds-WP_000593273.1, NZ_CP025268.1, 3094182, 3095132, +, 951, 50440; cds-WP _001053178.1, NZ_CP025268.1, 3095241, 3095804, +, 564, 161205; cds-WP_000017106.1, NZ_CP025268.1, 3095804, 3096220, +, 417, 22817; cds-WP_101348651.1, NZ_CP025268.1, 3096404, 3097384, -, 981, 191; cds-WP _000997795.1, NZ_CP025268.1, 3097402, 3098106, +, 705, 4937; cds-WP_001094831.1, NZ_CP025268.1, 3098124, 3098690, +, 567, 26149; cds-WP-_000994920.1, NZ_CP025268.1, 3098687, 3098977, +, 291, 17694; cds-WP _001174777.1, NZ_CP025268.1, 3098985, 3099578, +, 594, 32239; cds-WP _000239943.1, NZ_CP025268.1, 3099571, 3100707, +, 1137, 35490; cds-WP _000745210.1, NZ_CP025268.1, 3100862, 3101869, -, 1008, 1260; cds-WP _000394140.1, NZ_CP025268.1, 3101986, 3103032, -, 1047, 106075; cds-CXP41_RS16160, NZ_CP025268.1, 3103208, 3103928, -, 721, 97968; cds-WP _001107564.1, NZ_CP025268.1, 3104112, 3104438, -, 327, 6399; cds-WP_000786911.1, NZ_CP025268.1, 3104438, 3105157, -, 720, 20866; cds-WP_001321419.1, NZ_CP025268.1, 3105318, 3106370, +, 1053, 5717; cds-WP _000091700.1, NZ_CP025268.1, 3106398, 3106673, +, 276, 75717; cds-WP_000760323.1, NZ_CP025268.1, 3106738, 3107817, +, 1080, 141876; cds-WP_001049791.1, NZ_CP025268.1, 3108019, 3109275, +, 1257, 44999; cds-WP_001326492.1, NZ_CP025268.1, 3109325, 3111460, -, 2136, 5586; cds-WP_211180506.1, NZ_CP025268.1, 3111622, 3111705, -, 84, 0; cds-WP_000234514.1, NZ_CP025268.1, 3111858, 3112565, +, 708, 0; cds-WP_000942785.1, NZ_CP025268.1, 3112895, 3113431, -, 537, 15511; cds-CXP41_RS16225, NZ_CP025268.1, 3113433, 3114398, -, 966, 12998; cds-WP_000135077.1, NZ_CP025268.1, 3114359, 3115354, -, 996, 7; cds-WP _001324279.1, NZ_CP025268.1, 3115372, 3115782, -, 411, 106; cds-WP_001333829.1, NZ_CP025268.1, 3115848, 3116657, -, 810, 171203; cds-WP_101348652.1, NZ_CP025268.1, 3116855, 3121417, -, 4563, 2305837; cds-WP _000259302.1, NZ_CP025268.1, 3121902, 3123584, -, 1683, 0; cds-WP _000084131.1, NZ_CP025268.1, 3123939, 3126110, -, 2172, 1190; cds-WP _000853256.1, NZ_CP025268.1, 3126132, 3126536, -, 405, 1; cds-WP _001194661.1, NZ_CP025268.1, 3126541, 3127764, -, 1224, 217; cds-WP _000943100.1, NZ_CP025268.1, 3127775, 3128827, -, 1053, 0; cds-WP_000026117.1, NZ_CP025268.1, 3128827, 3130326, -, 1500, 0; cds-WP_001297764.1, NZ_CP025268.1, 3130577, 3131341, +, 765, 53; cds-WP_157825724.1, NZ_CP025268.1, 3131348, 3132484, -, 1137, 590; cds-WP _000019403.1-7, NZ_CP025268.1, 3132519, 3133499, +, 981, 201173; cds-CXP41_RS16300, NZ_CP025268.1, 3133649, 3134713, -, 1065, 25790; cds-WP_000339531.1, NZ_CP025268.1, 3134759, 3135517, -, 759, 0; cds-WP_001076243.1, NZ_CP025268.1, 3135549, 3136262, -, 714, 3; cds-CXP41_RS16315, NZ_CP025268.1, 3136436, 3137116, +, 681, 0; cds-WP_000933250.1, NZ_CP025268.1, 3137176, 3138675, -, 1500, 1; cds-WP_001297309.1, NZ_CP025268.1, 3138967, 3140826, -, 1860, 127; cds-WP _001295515.1, NZ_CP025268.1, 3141031, 3141897, +, 867, 8093; cds-WP_000334896.1, NZ_CP025268.1, 3142020, 3142268, -, 249, 8572; cds-WP_101348654.1, NZ_CP025268.1, 3142281, 3142622, -, 342, 0; cds-WP_000134014.1, NZ_CP025268.1, 3142615, 3143103, -, 489, 3463; cds-WP_001221939.1, NZ_CP025268.1, 3143096, 3143590, -, 495, 8381; cds-WP _000083065.1, NZ_CP025268.1, 3143590, 3145293, -, 1704, 17; cds-WP_000017703.1, NZ_CP025268.1, 3145290, 3146468, -, 1179, 0; cds-WP_001081870.1, NZ_CP025268.1, 3146458, 3147444, -, 987, 651139; cds-WP _000145410.1, NZ_CP025268.1, 3147447, 3148565, -, 1119, 1; cds-WP _001059136.1, NZ_CP025268.1, 3148754, 3149041, -, 288, 0; cds-CXP41_RS16385, NZ_CP025268.1, 3149160, 3150044, -, 885, 0; cds-WP_000262172.1, NZ_CP025268.1, 3150201, 3151241, +, 1041, 47254; cds-WP_000439331.1, NZ_CP025268.1, 3151281, 3151775, -, 495, 40220; cds-WP _000018760.1, NZ_CP025268.1, 3151966, 3152850, +, 885, 0; cds-WP_001240712.1, NZ_CP025268.1, 3153122, 3153547, -, 426, 3204; cds-WP_000527844.1, NZ_CP025268.1, 3153554, 3154288, -, 735, 210587; cds-WP _001301079.1, NZ_CP025268.1, 3154540, 3155727, +, 1188, 9; cds-WP _000268419.1, NZ_CP025268.1, 3155867, 3156526, +, 660, 18221; cds-WP_001407451.1, NZ_CP025268.1, 3156566, 3157465, -, 900, 811; cds-WP_001058802.1, NZ_CP025268.1, 3157659, 3158822, +, 1164, 148863; cds-WP_000013149.1, NZ_CP025268.1, 3158927, 3159754, +, 828, 171641; cds-WP_000691619.1, NZ_CP025268.1, 3159954, 3160880, +, 927, 42699; cds-WP _000848528.1, NZ_CP025268.1, 3160931, 3161188, +, 258, 74533; cds-WP _000095187.1, NZ_CP025268.1, 3161231, 3163450, -, 2220, 179330; cds-WP_101348655.1, NZ_CP025268.1, 3163561, 3164973, -, 1413, 34146; cds-WP_000965722.1, NZ_CP025268.1, 3165048, 3165785, -, 738, 7853; cds-WP _001281881.1, NZ_CP025268.1, 3166019, 3168277, -, 2259, 337489; cds-WP _001295629.1, NZ_CP025268.1, 3168415, 3170022, -, 1608, 0; cds-WP_000650107.1, NZ_CP025268.1, 3170155, 3170550, -, 396, 1614; cds-WP _000415584.1, NZ_CP025268.1, 3170552, 3170848, -, 297, 0; cds-WP_000183505.1, NZ_CP025268.1, 3171053, 3171535, -, 483, 367; cds-WP_000712658.1, NZ_CP025268.1, 3171588, 3171980, -, 393, 0; cds-WP _001221495.1, NZ_CP025268.1, 3172132, 3172791, +, 660, 5; cds-WP_000673402.1, NZ_CP025268.1, 3172788, 3174137, +, 1350, 59650; cds-WP _000917684.1, NZ_CP025268.1, 3174183, 3174515, -, 333, 0; cds-WP _000065430.1, NZ_CP025268.1, 3174834, 3175415, +, 582, 102496; cds-WP_000958598.1, NZ_CP025268.1, 3175446, 3175760, +, 315, 29; cds-WP _000195296.1, NZ_CP025268.1, 3175808, 3177700, -, 1893, 161962; cds-WP_000105733.1, NZ_CP025268.1, 3177729, 3178310, -, 582, 91985; cds-WP_101348656.1, NZ_CP025268.1, 3178310, 3179137, -, 828, 181992; cds-WP_000833393.1, NZ_CP025268.1, 3179162, 3179584, -, 423, 309; cds-WP_000917117.1, NZ_CP025268.1, 3179585, 3180214, -, 630, 1263; cds-WP _000831543.1, NZ_CP025268.1, 3180430, 3181101, +, 672, 98625; cds-WP _000442860.1, NZ_CP025268.1, 3181107, 3182267, +, 1161, 511910; cds-WP_001320780.1, NZ_CP025268.1, 3182305,
3183093, -, 789, 402; cds-WP_101348658.1, NZ_CP025268.1, 3183236, 3184009, +, 774, 51653; cds-WP_000469266.1, NZ_CP025268.1, 3184067, 3184237, -, 171, 281; cds-WP_001076997.1, NZ_CP025268.1, 3184499, 3185152, -, 654, 215838; cds-WP _001295626.1, NZ_CP025268.1, 3185526, 3185816, +, 291, 749; cds-WP _001272145.1, NZ_CP025268.1, 3186100, 3186651, +, 552, 1; cds-WP _088895425.1-5, NZ_CP025268.1;NZ_CP025268.1, 3186873;3187188, 3187188;3188101, +;+, 1229, 14838; cds-WP_101348659.1, NZ_CP025268.1, 3188086, 3190551, +, 2466, 0; cds-WP_001269824.1, NZ_CP025268.1, 3190567, 3191316, +, 750, 2; cds-WP _001269802.1, NZ_CP025268.1, 3191318, 3192382, +, 1065, 568; cds-WP_000350095.1, NZ_CP025268.1, 3192425, 3192625, -, 201, 0; cds-WP _000599926.1, NZ_CP025268.1, 3192894, 3193523, +, 630, 0; cds-WP_001311137.1, NZ_CP025268.1, 3193550, 3195211, +, 1662, 116570; cds-WP _001387081.1, NZ_CP025268.1, 3195452, 3195511, +, 60, 3; cds-WP _010723221.1, NZ_CP025268.1, 3195827, 3195886, +, 60, 8858; cds-WP _000869178.1, NZ_CP025268.1, 3196006, 3197439, -, 1434, 79885; cds-WP_001301081.1, NZ_CP025268.1, 3197487, 3200327, -, 2841, 288553; cds-WP _000046281.1, NZ_CP025268.1, 3200350, 3201651, -, 1302, 79013; cds-WP_001125331.1, NZ_CP025268.1, 3201893, 3202513, +, 621, 77954; cds-WP_000708487.1, NZ_CP025268.1, 3202577, 3203815, +, 1239, 154856; cds-WP _001281927.1, NZ_CP025268.1, 3203996, 3204817, -, 822, 206616; cds-WP _001295541.1, NZ_CP025268.1, 3204907, 3205275, -, 369, 795; cds-WP _001272796.1, NZ_CP025268.1, 3205380, 3205997, +, 618, 19410; cds-WP_000935206.1, NZ_CP025268.1, 3206010, 3206942, -, 933, 0; cds-WP _000986797.1, NZ_CP025268.1, 3207149, 3208060, +, 912, 0; cds-WP _000722957.1, NZ_CP025268.1, 3208057, 3208662, +, 606, 0; cds-WP_000804973.1, NZ_CP025268.1, 3208710, 3210173, +, 1464, 19; cds-WP_001264352.1, NZ_CP025268.1, 3210216, 3211229, -, 1014, 11; cds-WP _001144069.1, NZ_CP025268.1, 3211467, 3211682, +, 216, 73775; cds-WP_000918827.1, NZ_CP025268.1, 3211793, 3213538, +, 1746, 601564; cds-WP_000437376.1, NZ_CP025268.1, 3213733, 3215574, +, 1842, 1711864; cds-WP_000228937.1, NZ_CP025268.1, 3215653, 3216159, -, 507, 10768; cds-WP_001066494.1, NZ_CP025268.1, 3216413, 3217177, -, 765, 41; cds-WP_000018003.1, NZ_CP025268.1, 3217465, 3218088, +, 624, 8524; cds-WP_101348660.1, NZ_CP025268.1, 3218242, 3219762, -, 1521, 74; cds-WP_101348661.1, NZ_CP025268.1, 3220180, 3221559, +, 1380, 0; cds-WP_000450594.1, NZ_CP025268.1, 3221601, 3221933, -, 333, 0; cds-WP_000212475.1, NZ_CP025268.1, 3222152, 3223135, +, 984, 15223; cds-WP_001082856.1, NZ_CP025268.1, 3223319, 3226411, +, 3093, 9137; cds-WP _001219954.1, NZ_CP025268.1, 3226408, 3226857, +, 450, 0; cds-WP_001285432.1, NZ_CP025268.1, 3226920, 3228353, +, 1434, 0; cds-WP_000767652.1, NZ_CP025268.1, 3228487, 3229557, +, 1071, 0; cds-WP_000695487.1, NZ_CP025268.1, 3229574, 3231925, +, 2352, 1; cds-WP_000121433.1, NZ_CP025268.1, 3232351, 3234369, +, 2019, 2; cds-WP_000560266.1, NZ_CP025268.1, 3234414, 3234830, -, 417, 0; cds-WP_000550189.1, NZ_CP025268.1, 3234827, 3235141, -, 315, 118257; cds-WP _000018695.1, NZ_CP025268.1, 3235425, 3236561, -, 1137, 43020; cds-WP _001333820.1, NZ_CP025268.1, 3236646, 3237149, +, 504, 23; cds-WP_000942548.1, NZ_CP025268.1, 3237226, 3237918, +, 693, 0; cds-WP_000617698.1, NZ_CP025268.1, 3237997, 3238983, +, 987, 4; cds-WP _001098806.1, NZ_CP025268.1, 3239265, 3240230, +, 966, 7976; cds-WP_000211655.1, NZ_CP025268.1, 3240629, 3241873, +, 1245, 25200; cds-WP_000128135.1, NZ_CP025268.1, 3241878, 3242429, -, 552, 1; cds-WP_001199390.1, NZ_CP025268.1, 3242512, 3243999, -, 1488, 546; cds-WP_000187442.1, NZ_CP025268.1, 3244014, 3245426, -, 1413, 0; cds-WP_001226465.1, NZ_CP025268.1, 3245909, 3247207, +, 1299, 0; cds-WP_000406488.1, NZ_CP025268.1, 3247337, 3248113, +, 777, 234; cds-WP_211180508.1, NZ_CP025268.1, 3248353, 3248436, +, 84, 3; cds-WP_000422149.1, NZ_CP025268.1, 3248458, 3249120, +, 663, 633; cds-WP 001166760.1, NZ_CP025268.1, 3249124, 3249507, +, 384, 6; cds-WP_001295543.1, NZ_CP025268.1, 3249654, 3250022, +, 369, 7428; cds-WP_000031415.1, NZ_CP025268.1, 3250060, 3250365, +, 306, 7401; cds-WP_000785722.1, NZ_CP025268.1, 3250368, 3250772, +, 405, 0; cds-WP 000096091.1, NZ_CP025268.1, 3250762, 3251061, +, 300, 0; cds-WP 000732240.1, NZ_CP025268.1, 3251247, 3251639, +, 393, 0; cds-WP_000531213.1, NZ_CP025268.1, 3251709, 3252695, +, 987, 0; cds-WP 000384145.1, NZ_CP025268.1, 3252989, 3253354, +, 366, 320; cds-WP_001198807.1, NZ_CP025268.1, 3253596, 3253952, +, 357, 0; cds-WP_001041009.1, NZ_CP025268.1, 3254003, 3254899, -, 897, 120; cds-WP_000633576.1, NZ_CP025268.1, 3255004, 3255705, +, 702, 0; cds-WP_001345966.1, NZ_CP025268.1, 3255728, 3255892, +, 165, 0; cds-WP _000460499.1, NZ_CP025268.1, 3256026, 3257336, -, 1311, 1; cds-WP_000401598.1, NZ_CP025268.1, 3257364, 3258695, -, 1332, 4; cds-WP_000622126.1, NZ_CP025268.1, 3258970, 3260334, -, 1365, 72; cds-WP_000719990.1, NZ_CP025268.1, 3260406, 3260795, -, 390, 0; cds-WP _000861734.1, NZ_CP025268.1, 3260809, 3263103, -, 2295, 27471; cds-WP_001295545.1, NZ_CP025268.1, 3263137, 3264345, -, 1209, 0; cds-WP_000107723.1, NZ_CP025268.1, 3264371, 3265702, -, 1332, 544; cds-WP _000548347.1, NZ_CP025268.1, 3265724, 3266713, -, 990, 1; cds-WP _000104211.1, NZ_CP025268.1, 3266812, 3267750, -, 939, 1; cds-WP_001301311.1, NZ_CP025268.1, 3268065, 3268283, +, 219, 0; cds-WP_000675733.1, NZ_CP025268.1, 3268539, 3269078, +, 540, 0; cds-WP_101348662.1, NZ_CP025268.1, 3269100, 3270287, +, 1188, 17; cds-WP _001300387.1, NZ_CP025268.1, 3271310, 3272455, -, 1146, 0; cds-WP_101348723.1, NZ_CP025268.1, 3272552, 3273442, -, 891, 0; cds-WP_001058209.1, NZ_CP025268.1, 3273472, 3274242, -, 771, 0; cds-WP_000599636.1, NZ_CP025268.1, 3274258, 3275592, -, 1335, 0; cds-WP_001273753.1, NZ_CP025268.1, 3275967, 3277538, +, 1572, 0; cds-WP_001307405.1, NZ_CP025268.1, 3277687, 3278022, +, 336, 7; cds-WP _000347273.1, NZ_CP025268.1, 3278022, 3278486, +, 465, 40203; cds-WP_101348663.1, NZ_CP025268.1, 3278541, 3279350, -, 810, 12875; cds-WP _000681920.1, NZ_CP025268.1, 3279599, 3280879, +, 1281, 503; cds-WP _001336162.1, NZ_CP025268.1, 3280902, 3281375, +, 474, 0; cds-CXP41_RS17120, NZ_CP025268.1, 3281386, 3281757, +, 372, 0; cds-CXP41_RS17125, NZ_CP025268.1, 3281753, 3282310, +, 558, 0; cds-WP _001114869.1, NZ_CP025268.1, 3282661, 3283815, +, 1155, 2; cds-WP _000022766.1, NZ_CP025268.1, 3283828, 3284688, +, 861, 0; cds-WP _000203711.1, NZ_CP025268.1, 3284855, 3285331, +, 477, 0; cds-WP_000544489.1, NZ_CP025268.1, 3285370, 3286173, +, 804, 0; cds-WP_000534347.1, NZ_CP025268.1, 3286163, 3286954, +, 792, 0; cds-WP_001311150.1, NZ_CP025268.1, 3286955, 3287710, +, 756, 0; cds-WP _001045432.1, NZ_CP025268.1, 3288111, 3288695, +, 585, 0; cds-WP _000044775.1, NZ_CP025268.1, 3288775, 3289470, +, 696, 0; cds-WP_001355538.1, NZ_CP025268.1, 3289499, 3292015, +, 2517, 819; cds-WP_000816992.1, NZ_CP025268.1, 3292026, 3293117, +, 1092, 4; cds-WP_000809262.1, NZ_CP025268.1, 3293160, 3294020, -, 861, 97; cds-WP_000249104.1, NZ_CP025268.1, 3294085, 3296121, +, 2037, 82001; cds-WP_000246830.1, NZ_CP025268.1, 3296079, 3296474, +, 396, 185210; cds-WP_001158034.1, NZ_CP025268.1, 3296494, 3297084, +, 591, 13195; cds-WP_000646033.1, NZ_CP025268.1, 3297094, 3297669, +, 576, 40467; cds-WP_101348664.1, NZ_CP025268.1, 3297783, 3298823, -, 1041, 2855; cds-WP _001300423.1, NZ_CP025268.1, 3298896, 3299531, -, 636, 1; cds-WP_000037608.1, NZ_CP025268.1, 3299659, 3300177, +, 519, 3; cds-WP_000449030.1, NZ_CP025268.1, 3300157, 3300600, -, 444, 3; cds-WP_101348665.1, NZ_CP025268.1, 3300651, 3300953, +, 303, 4167; cds-WP_000908554.1, NZ_CP025268.1, 3300940, 3301443, -, 504, 3619; cds-WP _001295552.1, NZ_CP025268.1, 3301437, 3301961, -, 525, 19314; cds-WP_101348666.1, NZ_CP025268.1, 3302170, 3303165, +, 996, 0; cds-WP _001301318.1, NZ_CP025268.1, 3303174, 3304052, +, 879, 0; cds-WP_000130380.1, NZ_CP025268.1, 3304133, 3305140, +, 1008, 2405; cds-WP_000224351.1, NZ_CP025268.1, 3305258, 3306502, -, 1245, 139265; cds-WP_001295553.1, NZ_CP025268.1, 3306656, 3308545, -, 1890, 4688190; cds-WP_010723222.1, NZ_CP025268.1, 3308538, 3308618, -, 81, 200640; cds-WP _000802080.1, NZ_CP025268.1, 3308725, 3309609, -, 885, 1696468; cds-WP _001295554.1, NZ_CP025268.1, 3309717, 3311852, -, 2136, 4195203; cds-WP _000059466.1, NZ_CP025268.1, 3312099, 3312368, -, 270, 354048; cds-WP_000089698.1, NZ_CP025268.1, 3312517, 3313461, -, 945, 1082886; cds-WP _001040205.1, NZ_CP025268.1, 3313461, 3313862, -, 402, 72669; cds-WP _000133044.1, NZ_CP025268.1, 3314026, 3316698, -, 2673, 5975794; cds-WP _001031057.1, NZ_CP025268.1, 3316723, 3318210, -, 1488, 4193086; cds-WP_001300397.1, NZ_CP025268.1, 3318238, 3318690, -, 453, 711148; cds-WP_000207680.1, NZ_CP025268.1, 3319321, 3320664, +, 1344, 16940; cds-WP _001333576.1, NZ_CP025268.1, 3320672, 3322297, -, 1626, 13228; cds-WP_001295556.1, NZ_CP025268.1, 3322857, 3323189, -, 333, 112503; cds-WP _000071134.1, NZ_CP025268.1, 3323417, 3324754, -, 1338, 458764; cds-WP_000764731.1, NZ_CP025268.1, 3324747, 3325595, -, 849, 199698; cds-WP_001107467.1, NZ_CP025268.1, 3325685, 3327619, -, 1935, 2702686; cds-WP_000145975.1, NZ_CP025268.1, 3327719, 3328348, -, 630, 365240; cds-WP_0010544201, NZ_CP025268.1, 3328474, 3328767, +, 294, 28273; cds-WP _001148001.1, NZ_CP025268.1, 3328923, 3329399, -, 477, 39130; cds-WP_101348667.1, NZ_CP025268.1, 3329647, 3331044, +, 1398, 93391; cds-WP_000673575.1, NZ_CP025268.1, 3331267, 3332439, -, 1173, 556725; cds-WP 000813037.1, NZ_CP025268.1, 3332455, 3333420, -, 966, 183054; cds-WP_000940595.1, NZ_CP025268.1, 3333547, 3333804, -, 258, 756856; cds-WP_000271401.1, NZ_CP025268.1, 3333825, 3334136, -, 312, 844174; cds-WP_001047336.1, NZ_CP025268.1, 3334395, 3335366, +, 972, 8338; cds-WP _000445413.1, NZ_CP025268.1, 3335594, 3335872, +, 279, 2; cds-WP_000357259.1, NZ_CP025268.1, 3335920, 3337179, -, 1260, 70923; cds-WP_000429656.1, NZ_CP025268.1, 3337234, 3337488, -, 255, 30991; cds-WP_000004488.1, NZ_CP025268.1, 3337648, 3337941, -, 294, 94319; cds-WP_000476487.1, NZ_CP025268.1, 3337941, 3338576, -, 636, 302948; cds-WP_001296448.1, NZ_CP025268.1, 3338595, 3339146, -, 552, 204326; cds-WP _000925795.1, NZ_CP025268.1, 3339151, 3339933, -, 783, 368525; cds-WP _000438245.1, NZ_CP025268.1, 3339941, 3340750, -, 810, 608281; cds-WP_101348668.1, NZ_CP025268.1, 3340960, 3341937, +, 978, 65150; cds-WP_001295557.1, NZ_CP025268.1, 3341951, 3342937, +, 987, 22866; cds-WP _000030005.1, NZ_CP025268.1, 3342958, 3343524, +, 567, 36025; cds-WP _000030537.1, NZ_CP025268.1, 3343521, 3344096, +, 576, 142399; cds-WP_000669785.1, NZ_CP025268.1, 3344065, 3344622, +, 558, 302570; cds-WP_000224099.1, NZ_CP025268.1, 3344629, 3345354, +, 726, 299169; cds-WP _000809057.1, NZ_CP025268.1, 3345402, 3346835, +, 1434, 1320026; cds-WP_001176599.1, NZ_CP025268.1, 3346858, 3347145, +, 288, 171334; cds-WP _000183676.1, NZ_CP025268.1, 3347263, 3347754, +, 492, 275166; cds-WP_000243741.1, NZ_CP025268.1, 3347800, 3348654, +, 855, 440506; cds-WP_000216791.1, NZ_CP025268.1, 3348651, 3348923, +, 273, 261; cds-WP_000620405.1, NZ_CP025268.1, 3349137, 3349769, +, 633, 22089; cds-WP_000047091.1, NZ_CP025268.1, 3349766, 3350494, -, 729, 229183; cds-WP _001300411.1, NZ_CP025268.1, 3350491, 3351144, -, 654, 47032; cds-WP _000809774.1, NZ_CP025268.1, 3351374, 3353710, -, 2337, 138444; cds-WP _001299745.1, NZ_CP025268.1, 3353806, 3354735, -, 930, 0; cds-WP_001300352.1, NZ_CP025268.1, 3355410, 3359870, +, 4461, 48671; cds-WP_000081674.1, NZ_CP025268.1, 3359883, 3361301, +, 1419, 5; cds-WP_000465371.1, NZ_CP025268.1, 3361861, 3362625, +, 765, 0; cds-WP_001311157.1, NZ_CP025268.1, 3362797, 3363471, +, 675, 0; cds-WP _000914994.1, NZ_CP025268.1, 3363492, 3365873, +, 2382, 346; cds-CXP41_RS17555, NZ_CP025268.1, 3365870, 3366238, +, 369, 235; cds-WP_000019403.1-8, NZ_CP025268.1, 3366387, 3367367, -, 981, 136247; cds-CXP41_RS17565, NZ_CP025268.1, 3367435, 3367614, +, 180, 237; cds-WP_101348669.1, NZ_CP025268.1, 3367611, 3368327, +, 717, 0; cds-WP _000445111.1, NZ_CP025268.1, 3368512, 3369639, +,1128,166; cds-WP_000979882.1, NZ_CP025268.1, 3369699, 3370163, -, 465, 0; cds-WP_000209011.1, NZ_CP025268.1, 3370160, 3371035, -, 876, 0; cds-WP_000054239.1, NZ_CP025268.1, 3371032, 3371721, -, 690, 0; cds-WP_000108454.1, NZ_CP025268.1, 3371769, 3373259, -, 1491, 0; cds-WP _000224714.1, NZ_CP025268.1, 3373368, 3374261, -, 894, 0; cds-WP_000523845.1, NZ_CP025268.1, 3374383, 3375174, -, 792, 2272; cds-WP_000467018.1, NZ_CP025268.1, 3375554, 3376921,
+, 1368, 19275; cds-WP_000366129.1, NZ_CP025268.1, 3376964, 3377461, -, 498, 508575; cds-WP _000257293.1, NZ_CP025268.1, 3377467, 3378105, -, 639, 319459; cds-WP_000829818.1, NZ_CP025268.1, 3378500, 3378892, -, 393, 3193737; cds-WP _000847559.1, NZ_CP025268.1, 3378908, 3379336, -, 429, 2213122; cds-WP _001192332.1, NZ_CP025268.1, 3379555, 3380682, -, 1128, 29; cds-WP _001295270.1, NZ_CP025268.1, 3380876, 3381274, +, 399, 391; cds-WP _001295271.1, NZ_CP025268.1, 3381428, 3382795, +, 1368, 34946; cds-WP _000497723.1, NZ_CP025268.1, 3382885, 3383952, +, 1068, 90; cds-WP _001295272.1, NZ_CP025268.1, 3384015, 3384953, -, 939, 103868; cds-WP_001257846.1, NZ_CP025268.1, 3385388, 3385858, +, 471, 3823; cds-WP_000695690.1, NZ_CP025268.1, 3386223, 3386486, +, 264, 44265; cds-WP _001029013.1, NZ_CP025268.1, 3386542, 3386814, -, 273, 130769; cds-WP_000510991.1, NZ_CP025268.1, 3386906, 3388873, -, 1968, 97206; cds-WP _000854021.1, NZ_CP025268.1, 3388879, 3389811, -, 933, 0; cds-WP _000051841.1, NZ_CP025268.1, 3389819, 3390022, -, 204, 0; cds-WP _000440317.1, NZ_CP025268.1, 3390205, 3391134, +, 930, 220847; cds-WP_000055909.1, NZ_CP025268.1, 3391268, 3392713, -, 1446, 352552; cds-WP _001253618.1, NZ_CP025268.1, 3393143, 3396943, -, 3801, 549076; cds-WP _000123197.1, NZ_CP025268.1, 3397011, 3398480, -, 1470, 814142; cds-WP_000203105.1, NZ_CP025268.1, 3398470, 3399063, -, 594, 726515; cds-WP_000179409.1, NZ_CP025268.1, 3399072, 3399560, -, 489, 644355; cds-WP_000802511.1, NZ_CP025268.1, 3399560, 3400663, -, 1104, 389329; cds-WP_000913396.1, NZ_CP025268.1, 3400729, 3401772, -, 1044, 184553; cds-WP_001241469.1, NZ_CP025268.1, 3402077, 3404017, -, 1941, 41068; cds-WP_001148481.1, NZ_CP025268.1, 3404169, 3405143, +, 975, 14; cds-WP _000354622.1, NZ_CP025268.1, 3406121, 3406591, +, 471, 581733; cds-WP _000884639.1, NZ_CP025268.1, 3406602, 3407951, +, 1350, 1852492; cds-WP _000381173.1, NZ_CP025268.1, 3408060, 3408302, +, 243, 7175; cds-WP_001175728.1, NZ_CP025268.1, 3408292, 3409743, +, 1452, 230973; cds-WP_001145827.1, NZ_CP025268.1, 3409755, 3410636, +, 882, 178703; cds-WP_101348670.1, NZ_CP025268.1, 3410965, 3411930, +, 966, 1685262; cds-WP_000462905.1, NZ_CP025268.1, 3411956, 3412252, +, 297, 922298; cds-WP_001258895.1, NZ_CP025268.1, 3412338, 3413222, +, 885, 124269; cds-WP_001295275.1, NZ_CP025268.1, 3413306, 3413485, +, 180, 0; cds-WP _001129518.1, NZ_CP025268.1, 3413488, 3414150, -, 663, 0; cds-WP_000160334.1, NZ_CP025268.1, 3414549, 3415706, +, 1158, 0; cds-WP_001273238.1, NZ_CP025268.1, 3415718, 3418822, +, 3105, 22620; cds-WP_000825639.1, NZ_CP025268.1, 3419075, 3419296, +, 222, 0; cds-CXP41_RS17845, NZ_CP025268.1, 3419727, 3420751, +, 1025, 0; cds-WP _000019655.1, NZ_CP025268.1, 3420819, 3422000, +, 1182, 9; cds-WP_001300681.1, NZ_CP025268.1, 3422010, 3423113, +, 1104, 0; cds-WP_000078349.1, NZ_CP025268.1, 3423121, 3423879, +, 759, 11746; cds-WP _001286216.1, NZ_CP025268.1, 3429921, 3430475, +, 555, 75088; cds-WP _001070563.1, NZ_CP025268.1, 3430451, 3430708, -, 258, 19687; cds-WP_000451243.1, NZ_CP025268.1, 3430705, 3431523, -, 819, 7970; cds-WP_001301412.1, NZ_CP025268.1, 3431528, 3432100, -, 573, 8; cds-WP_001129722.1, NZ_CP025268.1, 3432105, 3432647, -, 543, 54922; cds-WP_000460680.1, NZ_CP025268.1, 3432676, 3433149, -, 474, 3080; cds-CXP41_RS17930, NZ_CP025268.1, 3433121, 3434246, -, 1126, 1826; cds-WP_074523692.1, NZ_CP025268.1, 3434376, 3434885, +, 510, 50646; cds-WP_101348671.1, NZ_CP025268.1, 3434900, 3435847, +, 948, 95755; cds-WP _000744778.1, NZ_CP025268.1, 3435893, 3437182, +, 1290, 492436; cds-WP _000691382.1, NZ_CP025268.1, 3437204, 3438580, +, 1377, 294961; cds-WP _000022442.1, NZ_CP025268.1, 3438710, 3439120, +, 411, 73587; cds-WP _000092696.1, NZ_CP025268.1, 3439117, 3439335, -, 219, 7276; cds-WP_000285607.1, NZ_CP025268.1, 3439391, 3439816, -, 426, 45643; cds-WP _000266486.1, NZ_CP025268.1, 3439827, 3440195, -, 369, 29066; cds-WP_001216368.1, NZ_CP025268.1, 3440302, 3440685, -, 384, 4815238; cds-WP_101348672.1, NZ_CP025268.1, 3440726, 3441715, -, 990, 21425773; cds-WP _000135224.1, NZ_CP025268.1, 3441741, 3442361, -, 621, 14335744; cds-WP _001029684.1, NZ_CP025268.1, 3442395, 3442784, -, 390, 6788459; cds-WP _000090775.1, NZ_CP025268.1, 3442801, 3443157, -, 357, 5624737; cds-WP _000868187.1, NZ_CP025268.1, 3443304, 3443420, -, 117, 1443665; cds-WP _001118861.1, NZ_CP025268.1, 3443452, 3444783, -, 1332, 27160434; cds-WP_001238914.1, NZ_CP025268.1, 3444791, 3445225, -, 435, 8105130; cds-WP_001140433.1, NZ_CP025268.1, 3445229, 3445408, -, 180, 5500590; cds-WP _000940121.1, NZ_CP025268.1, 3445412, 3445915, -, 504, 11853099; cds-WP_000358960.1, NZ_CP025268.1, 3445930, 3446283, -, 354, 9707218; cds-WP_000091945.1, NZ_CP025268.1, 3446293, 3446826, -, 534, 12462326; cds-WP _000062611.1, NZ_CP025268.1, 3446839, 3447231, -, 393, 6629373; cds-WP _001118930.1, NZ_CP025268.1, 3447265, 3447570, -, 306, 13871607; cds-WP _001096200.1, NZ_CP025268.1, 3447585, 3448124, -, 540, 14583055; cds-WP_000729185.1, NZ_CP025268.1, 3448139, 3448453, -, 315, 5440817; cds-WP _000613955.1, NZ_CP025268.1, 3448464, 3448835, -, 372, 3169150; cds-WP_000130100.1, NZ_CP025268.1, 3449000, 3449254, -, 255, 4010266; cds-WP _000644741.1, NZ_CP025268.1, 3449254, 3449445, -, 192, 3152131; cds-WP_000941212.1, NZ_CP025268.1, 3449445, 3449855, -, 411, 6449844; cds-WP _000529945.1, NZ_CP025268.1, 3449868, 3450569, -, 702, 7460363; cds-WP _000447529.1, NZ_CP025268.1, 3450587, 3450919, -, 333, 5778702; cds-WP _001138117.1, NZ_CP025268.1, 3450934, 3451212, -, 279, 6067094; cds-WP _000301864.1, NZ_CP025268.1, 3451229, 3452050, -, 822, 19182687; cds-WP_000617544.1, NZ_CP025268.1, 3452068, 3452370, -, 303, 4241425; cds-WP _000424395.1, NZ_CP025268.1, 3452367, 3452972, -, 606, 9972905; cds-WP_000579833.1, NZ_CP025268.1, 3452983, 3453612, -, 630, 8424155; cds-WP _001181004.1, NZ_CP025268.1, 3453645, 3453956, -, 312, 3967454; cds-WP_000461450.1, NZ_CP025268.1, 3454194, 3454613, -, 420, 0; cds-WP_000107576.1, NZ_CP025268.1, 3454615, 3456084, -, 1470, 5537; cds-WP_001142708.1, NZ_CP025268.1, 3456264, 3457079, +, 816, 0; cds-WP_101348673.1, NZ_CP025268.1, 3457063, 3459015, +, 1953, 0; cds-WP_001219894.1, NZ_CP025268.1, 3459025, 3460506, +, 1482, 11; cds-WP_001109573.1, NZ_CP025268.1, 3460503, 3461699, +, 1197, 103374; cds-WP_001202874.1, NZ_CP025268.1, 3461709, 3462146, +, 438, 0; cds-WP _001076046.1, NZ_CP025268.1, 3462154, 3462663, +, 510, 0; cds-WP_001041743.1, NZ_CP025268.1, 3462660, 3463037, +, 378, 0; cds-WP_000610634.1, NZ_CP025268.1, 3463030, 3463617, +, 588, 0; cds-WP_001058522.1, NZ_CP025268.1, 3463610, 3464593, +, 984, 0; cds-WP _001115238.1, NZ_CP025268.1, 3464608, 3465771, +, 1164, 7; cds-WP _001295161.1, NZ_CP025268.1, 3465768, 3466229, +, 462, 20; cds-WP_000178154.1, NZ_CP025268.1, 3466229, 3466906, +, 678, 245; cds-WP_101348674.1, NZ_CP025268.1, 3466935, 3467411, -, 477, 6809; cds-WP_000289085.1, NZ_CP025268.1, 3467483, 3467677, -, 195, 280; cds-WP_000773156.1, NZ_CP025268.1, 3467846, 3470539, -, 2694, 79667; cds-WP_000031783.1, NZ_CP025268.1, 3470831, 3472015, -, 1185, 19540468; cds-WP_000124700.1, NZ_CP025268.1, 3472086, 3474200, -, 2115, 44071846; cds-CXP41_RS18215, NZ_CP025268.1, 3474300, 3474767, -, 468, 7283746; cds-WP _000246815.1, NZ_CP025268.1, 3474864, 3475238, -, 375, 3561247; cds-WP _000903373.1, NZ_CP025268.1, 3475364, 3475651, -, 288, 21291; cds-WP _000820714.1, NZ_CP025268.1, 3475659, 3476018, -, 360, 3051; cds-WP _001209680.1, NZ_CP025268.1, 3476018, 3476404, -, 387, 4; cds-WP_000091466.1, NZ_CP025268.1, 3476404, 3477126, -, 723, 416398; cds-WP_000838250.1, NZ_CP025268.1, 3477293, 3478105, -, 813, 122690; cds-WP _001153615.1, NZ_CP025268.1, 3478326, 3478544, +, 219, 13749; cds-WP_000861334.1, NZ_CP025268.1, 3478593, 3479183, -, 591, 231338; cds-WP _001007730.1, NZ_CP025268.1, 3479278, 3479478, -, 201, 62097; cds-WP _000399147.1, NZ_CP025268.1, 3479488, 3481293, -, 1806, 46756; cds-WP _001297514.1, NZ_CP025268.1, 3481293, 3481844, -, 552, 13; cds-WP_101348675.1, NZ_CP025268.1, 3481975, 3483888, +, 1914, 99350; cds-WP _000057356.1, NZ_CP025268.1, 3483888, 3484910, +, 1023, 8870; cds-WP _000907085.1, NZ_CP025268.1, 3484904, 3485122, +, 219, 0; cds-WP _001274680.1, NZ_CP025268.1, 3485176, 3486045, +, 870, 20992; cds-WP_001148908.1, NZ_CP025268.1, 3486100, 3486504, -, 405, 0; cds-WP_000242755.1, NZ_CP025268.1, 3486806, 3487438, +, 633, 64107; cds-WP _001295162.1, NZ_CP025268.1, 3487489, 3489579, +, 2091, 142787; cds-WP_000963792.1, NZ_CP025268.1, 3489646, 3490866, -, 1221, 155720; cds-WP_000601847.1, NZ_CP025268.1, 3490952, 3491515, -, 564, 0; cds-WP _001280641.1, NZ_CP025268.1, 3491547, 3492149, -, 603, 27; cds-WP_000736862.1, NZ_CP025268.1, 3492139, 3492306, -, 168, 7465; cds-WP _000477225.1, NZ_CP025268.1, 3492411, 3492983, -, 573, 6139; cds-WP_000185247.1, NZ_CP025268.1, 3493254, 3494435, +, 1182, 9486; cds-WP_000049208.1, NZ_CP025268.1, 3494697, 3497240, +, 2544, 82176; cds-WP_000084764.1, NZ_CP025268.1, 3497237, 3497563, +, 327, 0; cds-WP_000493556.1, NZ_CP025268.1, 3497689, 3498495, +, 807, 0; cds-WP _000349855.1, NZ_CP025268.1, 3498514, 3499887, +, 1374, 492; cds-WP_001031834.1, NZ_CP025268.1, 3500134, 3500301, +, 168, 0; cds-WP _001311175.1, NZ_CP025268.1, 3500545, 3501933, +, 1389, 0; cds-WP_001295163.1, NZ_CP025268.1, 3501954, 3502976, +, 1023, 0; cds-WP_000847837.1, NZ_CP025268.1, 3503026, 3503856, +, 831, 0; cds-WP _000853353.1, NZ_CP025268.1, 3503853, 3504638, +, 786, 0; cds-WP _001276847.1, NZ_CP025268.1, 3504738, 3505469, +, 732, 43; cds-WP _000847144.1, NZ_CP025268.1, 3505621, 3506706, -, 1086, 2; cds-WP_000366857.1, NZ_CP025268.1, 3506718, 3508022, -, 1305, 0; cds-WP _000719728.1, NZ_CP025268.1, 3508034, 3508387, -, 354, 2; cds-CXP41_RS18420, NZ_CP025268.1, 3508398, 3509275, -, 878, 0; cds-WP_000090644.1, NZ_CP025268.1, 3509272, 3510498, -, 1227, 0; cds-WP_000497334.1, NZ_CP025268.1, 3510498, 3511661, -, 1164, 0; cds-WP_001295165.1, NZ_CP025268.1, 3511745, 3512107, -, 363, 0; cds-CXP41_RS18440, NZ_CP025268.1, 3512124, 3513030, -, 907, 3004; cds-WP _000165552.1, NZ_CP025268.1, 3513320, 3514324, -, 1005, 553136; cds-WP_001031729.1, NZ_CP025268.1, 3514317, 3515075, -, 759, 54147; cds-WP _000816280.1, NZ_CP025268.1, 3515068, 3515745, -, 678, 116283; cds-WP_101348676.1, NZ_CP025268.1, 3515763, 3516599, -, 837, 216262; cds-WP _000343215.1, NZ_CP025268.1, 3516706, 3517992, -, 1287, 788500; cds-WP_000439848.1, NZ_CP025268.1, 3518084, 3519172, -, 1089, 1098492; cds-WP_000818618.1, NZ_CP025268.1, 3519229, 3519750, -, 522, 90342; cds-WP _000815987.1, NZ_CP025268.1, 3520151, 3521389, -, 1239, 0; cds-WP_001264141.1, NZ_CP025268.1, 3521301, 3521705, -, 405, 0; cds-WP _001055759.1, NZ_CP025268.1, 3521695, 3522135, -, 441, 0; cds-WP _001069315.1, NZ_CP025268.1, 3522119, 3522658, -, 540, 0; cds-WP _001295166.1, NZ_CP025268.1, 3522658, 3523437, -, 780, 0; cds-WP _001336003.1, NZ_CP025268.1, 3523557, 3526109, +, 2553, 487247; cds-WP_000045744.1, NZ_CP025268.1, 3526275, 3526835, -, 561, 27543; cds-WP _000104533.1, NZ_CP025268.1, 3527155, 3529290, +, 2136, 245141; cds-WP _001295168.1, NZ_CP025268.1, 3529355, 3530023, +, 669, 56167; cds-WP_000660483.1, NZ_CP025268.1, 3530034, 3530435, +, 402, 71781; cds-WP_001135574.1, NZ_CP025268.1, 3530460, 3531338, +, 879, 668594; cds-WP _101348677.1, NZ_CP025268.1, 3531401, 3533125, -, 1725, 774; cds-WP _001265681.1, NZ_CP025268.1, 3533504, 3535126, +, 1623, 357011; cds-WP_101348678.1, NZ_CP025268.1, 3535202, 3536554, -, 1353, 109182; cds-WP_001157751.1, NZ_CP025268.1, 3536551, 3537270, -, 720, 41496; cds-WP _000856737.1, NZ_CP025268.1, 3537498, 3537974, +, 477, 5817; cds-WP _000980727.1, NZ_CP025268.1, 3538071, 3540392, +, 2322, 188770; cds-WP _001200455.1, NZ_CP025268.1, 3540849, 3541076, +, 228, 0; cds-WP _000737039.1, NZ_CP025268.1, 3541093, 3543414, +, 2322, 133782; cds-WP _000157586.1, NZ_CP025268.1, 3543414, 3543650, +, 237, 7435; cds-WP _000039062.1, NZ_CP025268.1, 3543853, 3544731, +, 879, 145662; cds-WP _001060070.1, NZ_CP025268.1, 3544760, 3545530, -, 771, 7479; cds-WP_001333371.1, NZ_CP025268.1, 3545568, 3546251, +, 684, 7797; cds-WP_000619389.1, NZ_CP025268.1, 3546310, 3546885, +, 576, 94524; cds-WP_001131758.1, NZ_CP025268.1, 3547245, 3548561, +, 1317, 38; cds-WP _000444342.1, NZ_CP025268.1, 3548672, 3550756, -, 2085, 3; cds-WP_000081909.1, NZ_CP025268.1, 3550766, 3553159, -, 2394, 354136; cds-WP _000906961.1, NZ_CP025268.1, 3553771, 3556476, +, 2706, 583570; cds-WP_101348679.1, NZ_CP025268.1, 3556519, 3557535, -,
1017, 2; cds-WP_001105504.1, NZ_CP025268.1, 3557539, 3558765, -, 1227, 0; cds-WP_001232948.1, NZ_CP025268.1, 3558954, 3560552, +, 1599, 1577; cds-WP _000815099.1, NZ_CP025268.1, 3560534, 3561292, -, 759, 267995; cds-WP_000928723.1, NZ_CP025268.1, 3561309, 3562139, -, 831, 173819; cds-WP _000371928.1, NZ_CP025268.1, 3562184, 3562510, -, 327, 873; cds-WP _000448136.1, NZ_CP025268.1, 3562700, 3564205, +, 1506, 184133; cds-CXP41_RS18680, NZ_CP025268.1, 3564474, 3564695, -, 222, 3; cds-WP_000993449.1, NZ_CP025268.1, 3564821, 3567268, -, 2448, 26; cds-WP _001197646.1, NZ_CP025268.1, 3567287, 3568720, -, 1434, 59; cds-WP _000253975.1, NZ_CP025268.1, 3568720, 3570015, -, 1296, 69711; cds-WP_000192523.1, NZ_CP025268.1, 3570033, 3572006, -, 1974, 32245; cds-WP_001283723.1, NZ_CP025268.1, 3572003, 3574189, -, 2187, 561341; cds-WP_211180509.1, NZ_CP025268.1, 3574347, 3574388, -, 42, 772; cds-WP_000799956.1, NZ_CP025268.1, 3574462, 3575565, -, 1104, 27065; cds-WP _001002544.1, NZ_CP025268.1, 3575758, 3576351, +, 594, 3539; cds-WP _000210111.1, NZ_CP025268.1, 3576408, 3577748, -, 1341, 1; cds-WP _000108330.1, NZ_CP025268.1, 3577752, 3578279, -, 528, 23300; cds-WP_000730252.1, NZ_CP025268.1, 3578418, 3579413, -, 996, 10304; cds-WP_000639811.1, NZ_CP025268.1, 3579637, 3580332, -, 696, 4602; cds-WP_000236293.1, NZ_CP025268.1, 3580455, 3581492, -, 1038, 339; cds-WP_001295206.1, NZ_CP025268.1, 3581825, 3582313, +, 489, 0; cds-WP_000065894.1, NZ_CP025268.1, 3582550, 3583728, +, 1179, 4011; cds-WP_000988497.1, NZ_CP025268.1, 3583725, 3584141, +, 417, 0; cds-CXP41_RS18765, NZ_CP025268.1, 3584170, 3584867, +, 698, 154869; cds-WP_001307436.1, NZ_CP025268.1, 3585091, 3585246, +, 156, 0; cds-WP_000634159.1, NZ_CP025268.1, 3585446, 3585730, +, 285, 0; cds-WP_000595082.1, NZ_CP025268.1, 3585768, 3587510, -, 1743, 0; cds-WP_101348680.1, NZ_CP025268.1, 3587630, 3588070, +, 441, 22; cds-WP_000073623.1, NZ_CP025268.1, 3588057, 3588800, -, 744, 468; cds-WP_000907792.1, NZ_CP025268.1, 3588797, 3589867, -, 1071, 66; cds-WP_101348681.1, NZ_CP025268.1, 3589869, 3590714, -, 846, 0; cds-WP_000099289.1, NZ_CP025268.1, 3590711, 3591598, -, 888, 0; cds-WP_000803190.1, NZ_CP025268.1, 3591696, 3593012, -, 1317, 1; cds-WP_000416891.1, NZ_CP025268.1, 3593411, 3594124, -, 714, 94799; cds-WP_000082101.1, NZ_CP025268.1, 3594126, 3594893, -, 768, 0; cds-WP_000803813.1, NZ_CP025268.1, 3594890, 3596167, -, 1278, 0; cds-WP_001295111.1, NZ_CP025268.1, 3596164, 3597090, -, 927, 1; cds-WP_000827696.1, NZ_CP025268.1, 3597138, 3598247, -, 1110, 33; cds-WP _000778768.1, NZ_CP025268.1, 3598671, 3599054, +, 384, 52; cds-WP_083586216.1, NZ_CP025268.1, 3599203, 3600345, -, 1143, 25947; cds-WP _000130217.1, NZ_CP025268.1, 3600616, 3601470, -, 855, 786295; cds-WP _001042003.1, NZ_CP025268.1, 3601715, 3602773, -, 1059, 81533; cds-WP _000617723.1, NZ_CP025268.1, 3602766, 3603434, -, 669, 31761; cds-WP_001040654.1, NZ_CP025268.1, 3603437, 3604930, -, 1494, 620350; cds-WP _000743193.1, NZ_CP025268.1, 3605080, 3605676, +, 597, 59019; cds-WP_001311191.1, NZ_CP025268.1, 3605666, 3605935, +, 270, 73316; cds-WP_000042895.1, NZ_CP025268.1, 3605938, 3606297, -, 360, 4; cds-WP _000964718.1, NZ_CP025268.1, 3606438, 3607064, +, 627, 14; cds-WP _000106551.1, NZ_CP025268.1, 3607138, 3609336, +, 2199, 415371; cds-WP _000130621.1, NZ_CP025268.1, 3609438, 3609683, -, 246, 9533; cds-WP _001100469.1, NZ_CP025268.1, 3609904, 3610569, +, 666, 3676; cds-WP _001245295.1, NZ_CP025268.1, 3610642, 3611199, +, 558, 568; cds-WP _001300943.1, NZ_CP025268.1, 3611203, 3612420, -, 1218, 348444; cds-WP_001300885.1, NZ_CP025268.1, 3612552, 3613601, +, 1050, 8; cds-WP_000285774.1, NZ_CP025268.1, 3613656, 3614243, +, 588, 9463; cds-WP _000953361.1, NZ_CP025268.1, 3614354, 3615928, +, 1575, 150060; cds-WP _000947068.1, NZ_CP025268.1, 3615928, 3616872, +, 945, 0; cds-WP _001008963.1, NZ_CP025268.1, 3616869, 3617702, +, 834, 0; cds-WP _001136229.1, NZ_CP025268.1, 3617702, 3618466, +, 765, 0; cds-WP _000173631.1, NZ_CP025268.1, 3618463, 3619269, +, 807, 0; cds-WP_001190062.1, NZ_CP025268.1, 3619275, 3619676, +, 402, 12; cds-WP _000015298.1, NZ_CP025268.1, 3619879, 3624114, +, 4236, 48; cds-WP _001271686.1, NZ_CP025268.1, 3624086, 3624469, +, 384, 904; cds-CXP41_RS18970, NZ_CP025268.1, 3624574, 3624924, +, 351, 1775; cds-WP _000420980.1, NZ_CP025268.1, 3625065, 3626201, +, 1137, 3903; cds-WP_001216257.1, NZ_CP025268.1, 3626366, 3627490, -, 1125, 620; cds-WP _000149156.1, NZ_CP025268.1, 3627490, 3630225, -, 2736, 223744; cds-WP _000361477.1, NZ_CP025268.1, 3630222, 3631289, -, 1068, 29; cds-WP_001296785.1, NZ_CP025268.1, 3631352, 3631465, -, 114, 9; cds-WP _001393583.1, NZ_CP025268.1, 3631655, 3633277, -, 1623, 0; cds-CXP41_RS19000, NZ_CP025268.1, 3633539, 3635145, -, 1607, 0; cds-WP_001028905.1, NZ_CP025268.1, 3635528, 3636580, +, 1053, 40894; cds-WP _000439170.1, NZ_CP025268.1, 3636895, 3638097, -, 1203, 14019; cds-WP _000902780.1, NZ_CP025268.1, 3638329, 3639828, +, 1500, 712468; cds-WP_000626187.1, NZ_CP025268.1, 3640072, 3640407, -, 336, 21411; cds-WP _000323571.1, NZ_CP025268.1, 3640798, 3641232, +, 435, 38644; cds-WP_211180511.1, NZ_CP025268.1, 3641299, 3641373, -, 75, 0; cds-WP_001098652.1, NZ_CP025268.1, 3641549, 3643018, +, 1470, 9271; cds-WP_000686620.1, NZ_CP025268.1, 3643067, 3643819, -, 753, 8680; cds-WP _001298719.1, NZ_CP025268.1, 3643827, 3645869, -, 2043, 165422; cds-WP_001300574.1, NZ_CP025268.1, 3646072, 3646914, +, 843, 158485; cds-WP_000160816.1, NZ_CP025268.1, 3646986, 3648338, +, 1353, 427736; cds-WP_001295215.1, NZ_CP025268.1, 3648392, 3648475, -, 84, 0; cds-WP _000008957.1, NZ_CP025268.1, 3649215, 3649568, +, 354, 0; cds-WP_000922639.1, NZ_CP025268.1, 3649622, 3650911, +, 1290, 9126; cds-WP_000065769.1, NZ_CP025268.1, 3650924, 3651349, +, 426, 0; cds-CXP41_RS19090, NZ_CP025268.1, 3651978, 3652724, +, 747, 51; cds-WP_000019403.1-9, NZ_CP025268.1, 3652869, 3653849, -, 981, 112221; cds-CXP41_RS19100, NZ_CP025268.1, 3653915, 3654293, +, 379, 91; cds-WP_001350553.1, NZ_CP025268.1, 3654648, 3655214, +, 567, 2; cds-WP _000478619.1, NZ_CP025268.1, 3655370, 3655900, +, 531, 0; cds-WP _001296814.1, NZ_CP025268.1, 3655942, 3656589, -, 648, 0; cds-WP_001298717.1, NZ_CP025268.1, 3656653, 3656979, -, 327, 0; cds-WP _000756550.1, NZ_CP025268.1, 3657095, 3657427, -, 333, 0; cds-WP_000965672.1, NZ_CP025268.1, 3657682, 3658254, +, 573, 0; cds-WP_000576690.1, NZ_CP025268.1, 3659053, 3659580, +, 528, 0; cds-WP _001205329.1, NZ_CP025268.1, 3659581, 3659859, -, 279, 0; cds-WP _001081984.1, NZ_CP025268.1, 3659919, 3661076, +, 1158, 0; cds-WP_000024872.1, NZ_CP025268.1, 3661101, 3664214, +, 3114, 25; cds-CXP41_RS19165, NZ_CP025268.1, 3664577, 3665306, -, 730, 22; cds-WP _001191059.1, NZ_CP025268.1, 3665674, 3666498, -, 825, 13; cds-WP_000372240.1, NZ_CP025268.1, 3666868, 3668268, -, 1401, 1; cds-WP _000784827.1, NZ_CP025268.1, 3668479, 3669876, -, 1398, 0; cds-WP _000934216.1, NZ_CP025268.1, 3670280, 3671929, +, 1650, 7; cds-WP_001167676.1, NZ_CP025268.1, 3671980, 3672582, -, 603, 88; cds-WP _000357790.1, NZ_CP025268.1, 3673102, 3674001, +, 900, 0; cds-WP _000191237.1, NZ_CP025268.1, 3674050, 3675063, +, 1014, 2293; cds-WP _001149003.1, NZ_CP025268.1, 3675474, 3676796, +, 1323, 18288; cds-WP_001296794.1, NZ_CP025268.1, 3676978, 3679038, -, 2061, 378923; cds-WP _001295219.1, NZ_CP025268.1, 3679108, 3679875, -, 768, 18291; cds-WP_000037562.1, NZ_CP025268.1, 3680107, 3681036, +, 930, 259499; cds-WP_001163141.1, NZ_CP025268.1, 3681132, 3682628, -, 1497, 456221; cds-WP _000858214.1, NZ_CP025268.1, 3682849, 3684135, -, 1287, 95537; cds-WP _001266293.1, NZ_CP025268.1, 3684318, 3686306, -, 1989, 17842; cds-WP_001225124.1, NZ_CP025268.1, 3686388, 3689861, -, 3474, 0; cds-WP _001311203.1, NZ_CP025268.1, 3689843, 3690949, -, 1107, 324636; cds-WP _001407405.1, NZ_CP025268.1, 3690956, 3693295, -, 2340, 581387; cds-WP_101348682.1, NZ_CP025268.1, 3693306, 3695924, -, 2619, 0; cds-CXP41_RS19270, NZ_CP025268.1, 3695921, 3696673, -, 753, 87665; cds-WP_001063318.1, NZ_CP025268.1, 3696685, 3696873, -, 189, 208; cds-WP _001204931.1, NZ_CP025268.1, 3697146, 3698717, +, 1572, 19967; cds-WP_000988308.1, NZ_CP025268.1, 3698714, 3698905, +, 192, 19; cds-WP _000191622.1, NZ_CP025268.1, 3698902, 3700581, +, 1680, 372; cds-WP _000141634.1, NZ_CP025268.1, 3700668, 3700775, -, 108, 0; cds-WP_001295225.1, NZ_CP025268.1, 3701251, 3702522, +, 1272, 48354; cds-WP_000107012.1, NZ_CP025268.1, 3702552, 3703556, -, 1005, 0; cds-WP_001196486.1, NZ_CP025268.1, 3703553, 3704536, -, 984, 506; cds-WP_000084677.1, NZ_CP025268.1, 3704547, 3705449, -, 903, 56398; cds-WP _000938855.1, NZ_CP025268.1, 3705459, 3706478, -, 1020, 15949; cds-WP _001222883.1, NZ_CP025268.1, 3706786, 3708393, -, 1608, 21819; cds-CXP41_RS19355, NZ_CP025268.1, 3709472, 3711164, -, 1693, 137260; cds-WP _000189019.1, NZ_CP025268.1, 3711488, 3712696, -, 1209, 0; cds-WP _001299523.1, NZ_CP025268.1, 3712925, 3713623, -, 699, 28036; cds-WP_000438957.1, NZ_CP025268.1, 3713781, 3714344, +, 564, 60178; cds-WP _000617473.1, NZ_CP025268.1, 3714341, 3714781, +, 441, 462; cds-WP _000013950.1, NZ_CP025268.1, 3714750, 3717083, -, 2334, 8010; cds-WP _000747625.1, NZ_CP025268.1, 3717236, 3717895, +, 660, 9381; cds-WP_000805038.1, NZ_CP025268.1, 3717999, 3718973, +, 975, 8121; cds-WP _000190516.1, NZ_CP025268.1, 3719023, 3719733, -, 711, 140496; cds-WP _000455798.1, NZ_CP025268.1, 3720167, 3720457, +, 291, 0; cds-WP_000014594.1, NZ_CP025268.1, 3720738, 3720950, +, 213, 530883; cds-WP_211180513.1, NZ_CP025268.1, 3721089, 3721160, +, 72, 780; cds-WP _001135732.1, NZ_CP025268.1, 3721137, 3721289, -, 153, 5775; cds-WP _085947770.1, NZ_CP025268.1;NZ_CP025268.1, 3721369;3721849, 3721849;3722738, +;+, 1370, 99448; cds-WP_001291772.1, NZ_CP025268.1, 3723017, 3725086, -, 2070, 1777903; cds-WP_001168560.1, NZ_CP025268.1, 3725096, 3726007, -, 912, 106820; cds-WP _000980107.1, NZ_CP025268.1, 3726102, 3726401, -, 300, 0; cds-WP_001182650.1, NZ_CP025268.1, 3726576, 3727571, +, 996, 0; cds-WP _001296808.1, NZ_CP025268.1, 3727613, 3728050, -, 438, 0; cds-WP _001295228.1, NZ_CP025268.1, 3728096, 3728437, -, 342, 0; cds-CXP41_RS19455, NZ_CP025268.1, 3728606, 3730062, -, 1457, 0; cds-WP _001149591.1, NZ_CP025268.1, 3730134, 3731456, -, 1323, 0; cds-WP_101348684.1, NZ_CP025268.1, 3731822, 3732814, +, 993, 0; cds-WP_101348685.1, NZ_CP025268.1, 3732892, 3734433, +, 1542, 0; cds-WP _000045978.1, NZ_CP025268.1, 3734411, 3735592, +, 1182, 0; cds-WP _000494484.1, NZ_CP025268.1, 3735670, 3736848, +, 1179, 24833; cds-WP _001297957.1, NZ_CP025268.1, 3737045, 3737869, -, 825, 231322; cds-WP_000761225.1, NZ_CP025268.1, 3738189, 3740219, +, 2031, 171185; cds-WP_000144363.1, NZ_CP025268.1, 3740397, 3741650, +, 1254, 8752; cds-WP _001300918.1, NZ_CP025268.1, 3741801, 3742274, -, 474, 4; cds-WP _000514240.1, NZ_CP025268.1, 3742376, 3743224, -, 849, 6; cds-WP_000053415.1, NZ_CP025268.1, 3743406, 3744554, +, 1149, 350138; cds-WP_000869037.1, NZ_CP025268.1, 3744655, 3745653, +, 999, 0; cds-WP _000576079.1, NZ_CP025268.1, 3745665, 3746132, +, 468, 0; cds-WP _000721664.1, NZ_CP025268.1, 3746250, 3746723, +, 474, 0; cds-WP_000279599.1, NZ_CP025268.1, 3746726, 3748003, +, 1278, 370; cds-WP _000776887.1, NZ_CP025268.1, 3748016, 3749002, +, 987, 0; cds-WP _000196054.1, NZ_CP025268.1, 3749006, 3750502, +, 1497, 0; cds-WP _000089481.1, NZ_CP025268.1, 3750499, 3751161, +, 663, 0; cds-WP_001350555.1, NZ_CP025268.1, 3751154, 3752014, +, 861, 0; cds-WP_101348686.1, NZ_CP025268.1, 3752008, 3752703, +, 696, 0; cds-CXP41_RS24405, NZ_CP025268.1, 3752735, 3752836, -, 102, 0; cds-CXP41_RS19560, NZ_CP025268.1, 3752840, 3753028, +, 189, 0; cds-WP _000906819.1, NZ_CP025268.1, 3753050, 3753790, -, 741, 0; cds-WP_000164036.1, NZ_CP025268.1, 3753914, 3754888, +, 975, 0; cds-WP_000364884.1, NZ_CP025268.1, 3754885, 3756021, -, 1137, 0; cds-WP_000478195.1, NZ_CP025268.1, 3756027, 3756350, -, 324, 408; cds-WP_001066080.1, NZ_CP025268.1, 3756533, 3756676, +, 144, 0; cds-WP_000183980.1, NZ_CP025268.1, 3756895, 3758433, -, 1539, 712; cds-WP_000741518.1, NZ_CP025268.1, 3758598, 3759749, -, 1152, 0; cds-WP_000582468.1, NZ_CP025268.1, 3759939, 3761783, -, 1845, 127680; cds-WP _000206275.1, NZ_CP025268.1, 3761780, 3763171, -, 1392, 94584; cds-WP_101348687.1, NZ_CP025268.1, 3763269, 3763877, -, 609, 3628; cds-WP_000015300.1, NZ_CP025268.1, 3764105, 3768238, +, 4134, 60; cds-WP_000072850.1, NZ_CP025268.1, 3768259, 3769101, +, 843, 128; cds-CXP41_RS24295, NZ_CP025268.1, 3769129, 3770087, +, 959, 5; cds-WP_000642478.1, NZ_CP025268.1, 3770099, 3770560, +, 462, 0; cds-
WP_001326561.1, NZ_CP025268.1, 3771267, 3771602, +, 336, 0; cds-WP_000364939.1, NZ_CP025268.1, 3772165, 3773301, -, 1137, 0; cds-WP_000479624.1, NZ_CP025268.1, 3773304, 3773666, -, 363, 0; cds-WP_101348688.1, NZ_CP025268.1, 3774203, 3776116, +, 1914, 67758; cds-WP_000645439.1, NZ_CP025268.1, 3776346, 3777494, +, 1149, 4850; cds-WP_000228269.1, NZ_CP025268.1, 3777494, 3778081, +, 588, 52547; cds-WP_000517100.1, NZ_CP025268.1, 3778093, 3778302, -, 210, 21564; cds-WP _000665680.1, NZ_CP025268.1, 3778587, 3778949, +, 363, 5103; cds-WP_001295233.1, NZ_CP025268.1, 3779321, 3780976, +, 1656, 2932; cds-WP_000636500.1, NZ_CP025268.1, 3780976, 3781752, +, 777, 2; cds-WP_000586962.1, NZ_CP025268.1, 3781749, 3782939, +, 1191, 0; cds-WP _000932347.1, NZ_CP025268.1, 3783137, 3783610, +, 474, 32261; cds-WP_001277561.1, NZ_CP025268.1, 3783663, 3784484, -, 822, 219629; cds-WP_001076194.1, NZ_CP025268.1, 3784564, 3785583, -, 1020, 372722; cds-WP _000003377.1, NZ_CP025268.1, 3785583, 3786050, -, 468, 403389; cds-WP_000024392.1, NZ_CP025268.1, 3786113, 3786364, -, 252, 386813; cds-WP _001156181.1, NZ_CP025268.1, 3786506, 3786937, -, 432, 223624; cds-WP_001350558.1, NZ_CP025268.1, 3787182, 3788726, +, 1545, 248455; cds-WP_001214147.1, NZ_CP025268.1, 3788736, 3790019, +, 1284, 212648; cds-WP_101348689.1, NZ_CP025268.1, 3790023, 3790982, +, 960, 1332; cds-WP_101348690.1, NZ_CP025268.1, 3790969, 3792003, -, 1035, 26; cds-WP _000646007.1, NZ_CP025268.1, 3792242, 3793267, -, 1026, 21437; cds-WP_001213834.1, NZ_CP025268.1, 3793277, 3794473, -, 1197, 20561; cds-WP_000842820.1, NZ_CP025268.1, 3794748, 3795605, -, 858, 926; cds-WP_000587764.1, NZ_CP025268.1, 3795909, 3796841, +, 933, 389746; cds-WP_000699219.1, NZ_CP025268.1, 3796851, 3797897, +, 1047, 80152; cds-WP_001264584.1, NZ_CP025268.1, 3797901, 3798860, +, 960, 304626; cds-WP_001395405.1, NZ_CP025268.1, 3798870, 3800129, +, 1260, 142366; cds-WP _001236433.1, NZ_CP025268.1, 3800161, 3801234, -, 1074, 37840; cds-WP_000790279.1, NZ_CP025268.1, 3801267, 3802118, -, 852, 131193; cds-WP_000615254.1, NZ_CP025268.1, 3802189, 3802887, -, 699, 33444; cds-WP_000376841.1, NZ_CP025268.1, 3802905, 3803921, -, 1017, 114374; cds-WP_001188013.1, NZ_CP025268.1, 3803961, 3804980, -, 1020, 16033; cds-WP_000683964.1, NZ_CP025268.1, 3804980, 3806059, -, 1080, 42794; cds-WP_000158225.1, NZ_CP025268.1, 3806103, 3807038, -, 936, 41950; cds-WP _000229840.1, NZ_CP025268.1, 3807075, 3807872, -, 798, 43529; cds-WP_000634283.1, NZ_CP025268.1, 3807865, 3808989, -, 1125, 168272; cds-WP_001350560.1, NZ_CP025268.1, 3808986, 3810056, -, 1071, 10833; cds-WP_000891564.1, NZ_CP025268.1, 3810462, 3811739, +, 1278, 186522; cds-WP_001171866.1, NZ_CP025268.1, 3811747, 3812226, +, 480, 1810; cds-WP_001114543.1, NZ_CP025268.1, 3812265, 3813074, -, 810, 11585; cds-WP_001051798.1, NZ_CP025268.1, 3813172, 3813339, -, 168, 44298; cds-WP _000091955.1, NZ_CP025268.1, 3813360, 3813596, -, 237, 208948; cds-WP_001350561.1, NZ_CP025268.1, 3813813, 3814481, -, 669, 19016; cds-WP_000050139.1, NZ_CP025268.1, 3814653, 3815873, +, 1221, 1583; cds-WP_000976073.1, NZ_CP025268.1, 3815851, 3816309, +, 459, 4711; cds-WP_000818601.1, NZ_CP025268.1, 3816416, 3817012, +, 597, 189030; cds-WP_000806177.1, NZ_CP025268.1, 3817049, 3817690, -, 642, 1; cds-CXP41_RS19895, NZ_CP025268.1, 3817757, 3818472, -, 716, 5364; cds-WP_000621340.1, NZ_CP025268.1, 3818599, 3819462, +, 864, 16906; cds-WP_001350563.1, NZ_CP025268.1, 3819683, 3820507, +, 825, 3956; cds-WP _000924289.1, NZ_CP025268.1, 3820797, 3821414, +, 618, 2016; cds-WP _000870036.1, NZ_CP025268.1, 3821411, 3823093, -, 1683, 0; cds-WP_001295237.1, NZ_CP025268.1, 3823351, 3823974, +, 624, 490712; cds-WP _000135058.1, NZ_CP025268.1, 3824029, 3824304, +, 276, 172550; cds-WP _000280488.1, NZ_CP025268.1, 3824323, 3826431, +, 2109, 2010454; cds-WP _001070177.1, NZ_CP025268.1, 3826438, 3827127, +, 690, 173338; cds-WP_000678419.1, NZ_CP025268.1, 3827133, 3829214, +, 2082, 761330; cds-WP_000468833.1, NZ_CP025268.1, 3829383, 3830588, -, 1206, 34023; cds-WP _001295238.1, NZ_CP025268.1, 3830868, 3832259, +, 1392, 315899; cds-WP _001300954.1, NZ_CP025268.1, 3832380, 3834089, +, 1710, 13; cds-WP_000702898.1, NZ_CP025268.1, 3834142, 3836460, -, 2319, 3; cds-WP_000834439.1, NZ_CP025268.1, 3836470, 3837852, -, 1383, 11; cds-WP_001172878.1, NZ_CP025268.1, 3838876, 3840060, +, 1185, 1686; cds-WP _000535961.1, NZ_CP025268.1, 3840171, 3841094, +, 924, 9630; cds-WP _000779426.1, NZ_CP025268.1, 3841098, 3841916, -, 819, 16966; cds-WP_000805509.1, NZ_CP025268.1, 3842138, 3842431, +, 294, 17; cds-WP_001288549.1, NZ_CP025268.1, 3842472, 3843662, -, 1191, 124758; cds-WP _001295241.1, NZ_CP025268.1, 3843873, 3844325, -, 453, 0; cds-WP_001403836.1, NZ_CP025268.1, 3844378, 3845712, -, 1335, 0; cds-WP_001065718.1, NZ_CP025268.1, 3845887, 3847653, +,1767,1395; cds-WP_000879194.1, NZ_CP025268.1, 3847699, 3849090, -, 1392, 0; cds-WP_000936566.1, NZ_CP025268.1, 3849228, 3850547, -, 1320, 557; cds-CXP41_RS20045, NZ_CP025268.1, 3850557, 3852058, -, 1502, 217; cds-WP_000633668.1, NZ_CP025268.1, 3852058, 3852648, -, 591, 96732; cds-WP_001181706.1, NZ_CP025268.1, 3852724, 3853014, -, 291, 1608; cds-WP _000168475.1, NZ_CP025268.1, 3853018, 3854706, -, 1689, 631; cds-WP _001300753.1, NZ_CP025268.1, 3854812, 3854910, -, 99, 105; cds-WP_001054909.1, NZ_CP025268.1, 3855475, 3855564, +, 90, 0; cds-WP_211180519.1, NZ_CP025268.1, 3855688, 3855762, -, 75, 0; cds-WP _000828746.1, NZ_CP025268.1, 3855844, 3857028, +, 1185, 49; cds-WP _000148061.1, NZ_CP025268.1, 3857036, 3857533, -, 498, 32; cds-WP _074557320.1, NZ_CP025268.1, 3857530, 3857892, -, 363, 9; cds-WP_000703959.1, NZ_CP025268.1, 3857882, 3858229, -, 348, 0; cds-WP_000511289.1, NZ_CP025268.1, 3858337, 3858786, +, 450, 2; cds-WP _000828527.1, NZ_CP025268.1, 3858833, 3860326, -, 1494, 0; cds-WP _001087145.1, NZ_CP025268.1, 3860323, 3862038, -, 1716, 0; cds-WP _001013540.1, NZ_CP025268.1, 3862205, 3863098, +, 894, 0; cds-CXP41_RS20135, NZ_CP025268.1, 3863095, 3863909, -, 815, 0; cds-CXP41_RS20140, NZ_CP025268.1, 3863909, 3865521, -, 1613, 5; cds-WP_001300779.1, NZ_CP025268.1, 3865817, 3866533, +, 717, 0; cds-WP_001279752.1, NZ_CP025268.1, 3866530, 3868191, -, 1662, 2753; cds-WP_001243431.1, NZ_CP025268.1, 3868387, 3868815, -, 429, 35; cds-WP_001243437.1, NZ_CP025268.1, 3868927, 3869340, -, 414, 16367; cds-WP _000620888.1, NZ_CP025268.1, 3869646, 3869978, +, 333, 82; cds-WP _001336371.1, NZ_CP025268.1, 3869980, 3871194, -, 1215, 3; cds-WP _001340434.1, NZ_CP025268.1, 3871295, 3872359, +, 1065, 0; cds-WP _000253455.1, NZ_CP025268.1, 3872356, 3873693, -, 1338, 1951; cds-WP _000705001.1, NZ_CP025268.1, 3873768, 3874916, -, 1149, 766; cds-WP _001198722.1, NZ_CP025268.1, 3874913, 3875530, -, 618, 0; cds-WP _000127091.1, NZ_CP025268.1, 3875514, 3876392, -, 879, 0; cds-WP_000174305.1, NZ_CP025268.1, 3876389, 3877078, -, 690, 0; cds-WP_000772934.1, NZ_CP025268.1, 3877356, 3878012, +, 657, 82; cds-WP _000985549.1, NZ_CP025268.1, 3878058, 3878870, -, 813, 112987; cds-WP _000522208.1, NZ_CP025268.1, 3878985, 3879383, -, 399, 1; cds-WP _000072067.1, NZ_CP025268.1, 3879623, 3882037, -, 2415, 563499; cds-WP _000060112.1, NZ_CP025268.1, 3882066, 3883139, -, 1074, 435621; cds-WP _000673464.1, NZ_CP025268.1, 3883139, 3884239, -, 1101, 245630; cds-WP _000059111.1, NZ_CP025268.1, 3884244, 3885647, -, 1404, 155359; cds-WP_120795392.1, NZ_CP025268.1, 3885944, 3886024, +, 81, 0; cds-WP_000831330.1, NZ_CP025268.1, 3886254, 3886394, +, 141, 89214; cds-WP _000239730.1, NZ_CP025268.1, 3886411, 3886770, +, 360, 511738; cds-WP _001307474.1, NZ_CP025268.1, 3886734, 3886991, +, 258, 714363; cds-WP _000378250.1, NZ_CP025268.1, 3886994, 3888640, +, 1647, 3228559; cds-WP _001282346.1, NZ_CP025268.1, 3888746, 3890110, +, 1365, 225559; cds-WP _001364348.1, NZ_CP025268.1, 3890353, 3890427, +, 75, 0; cds-WP_001295247.1, NZ_CP025268.1, 3890648, 3892063, +, 1416, 0; cds-WP_000131925.1, NZ_CP025268.1, 3892154, 3893401, +, 1248, 0; cds-WP_101348691.1, NZ_CP025268.1, 3893533, 3894708, +, 1176, 23464; cds-WP _001311238.1, NZ_CP025268.1, 3894683, 3895642, +, 960, 47438; cds-WP _001296581.1, NZ_CP025268.1, 3895799, 3896548, +, 750, 99465; cds-WP _001291268.1, NZ_CP025268.1, 3896570, 3897136, +, 567, 29417; cds-WP_000019348.1, NZ_CP025268.1, 3897190, 3898527, -, 1338, 644268; cds-CXP41_RS20310, NZ_CP025268.1, 3898692, 3899358, +, 667, 0; cds-WP_000116777.1, NZ_CP025268.1, 3899425, 3899892, +, 468, 1; cds-WP_000193072.1, NZ_CP025268.1, 3899941, 3900528, +, 588, 0; cds-WP _000766900.1, NZ_CP025268.1, 3900590, 3901312, -, 723, 0; cds-WP_001336377.1, NZ_CP025268.1, 3901327, 3902496, -, 1170, 0; cds-WP_000489881.1, NZ_CP025268.1, 3902523, 3904139, -, 1617, 0; cds-WP_000643228.1, NZ_CP025268.1, 3904208, 3905620, -, 1413, 0; cds-WP _000137296.1, NZ_CP025268.1, 3905639, 3907516, -, 1878, 280602; cds-WP _001295251.1, NZ_CP025268.1, 3907650, 3908486, -, 837, 0; cds-WP_000377786.1, NZ_CP025268.1, 3908772, 3909497, -, 726, 205679; cds-WP _000063125.1, NZ_CP025268.1, 3909512, 3910285, -, 774, 239107; cds-WP _001251998.1, NZ_CP025268.1, 3910468, 3911358, -, 891, 168777; cds-WP _000741620.1, NZ_CP025268.1, 3911358, 3912317, -, 960, 49866; cds-WP_000867146.1, NZ_CP025268.1, 3912404, 3913444, -, 1041, 1616; cds-WP _047397264.1, NZ_CP025268.1, 3913758, 3915587, -, 1830, 3647621; cds-WP _000933736.1, NZ_CP025268.1, 3915749, 3917119, -, 1371, 1025329; cds-WP _001251965.1, NZ_CP025268.1, 3917472, 3917891, -, 420, 576702; cds-WP _000190506.1, NZ_CP025268.1, 3917912, 3919294, -, 1383, 2956632; cds-WP_000896498.1, NZ_CP025268.1, 3919321, 3920184, -, 864, 4171310; cds-WP _001176745.1, NZ_CP025268.1, 3920235, 3921776, -, 1542, 4640035; cds-WP_001288587.1, NZ_CP025268.1, 3921789, 3922322, -, 534, 1048830; cds-WP _001052219.1, NZ_CP025268.1, 3922337, 3922807, -, 471, 2185721; cds-WP_000429386.1, NZ_CP025268.1, 3922869, 3923108, -, 240, 537530; cds-WP_000135625.1, NZ_CP025268.1, 3923155, 3923970, -, 816, 2075845; cds-WP _000116695.1, NZ_CP025268.1, 3923979, 3924359, -, 381, 246809; cds-WP _000932839.1, NZ_CP025268.1, 3924976, 3925599, -, 624, 40092; cds-WP_000499788.1, NZ_CP025268.1, 3925663, 3927552, -, 1890, 1333383; cds-WP _000763724.1, NZ_CP025268.1, 3927931, 3928374, -, 444, 153344; cds-WP_000432970.1, NZ_CP025268.1, 3928464, 3928922, -, 459, 0; cds-WP_000845104.1, NZ_CP025268.1, 3929074, 3930066, +, 993, 6360; cds-WP _000956642.1, NZ_CP025268.1, 3930071, 3931522, -, 1452, 1905; cds-WP _001299914.1, NZ_CP025268.1, 3931516, 3933012, -, 1497, 0; cds-WP_000102319.1, NZ_CP025268.1, 3933235, 3935103, +, 1869, 24613; cds-WP_001301979.1, NZ_CP025268.1, 3935270, 3935689, +, 420, 18; cds-WP _000387770.1, NZ_CP025268.1, 3935697, 3937202, +, 1506, 185324; cds-WP _000211858.1, NZ_CP025268.1, 3937207, 3938172, +, 966, 90371; cds-WP_001056271.1, NZ_CP025268.1, 3938197, 3939087, +, 891, 149749; cds-WP _001300603.1, NZ_CP025268.1, 3939213, 3940142, +, 930, 155374; cds-WP _000224470.1, NZ_CP025268.1, 3940146, 3941138, +, 993, 264441; cds-WP_101348692.1, NZ_CP025268.1, 3941104, 3942531, -, 1428, 5063; cds-WP _001131164.1, NZ_CP025268.1, 3942554, 3943246, -, 693, 276; cds-WP _000379245.1, NZ_CP025268.1, 3949047, 3949886, -, 840, 76707; cds-WP_000841001.1, NZ_CP025268.1, 3950005, 3950343, +, 339, 98628; cds-WP_000058964.1, NZ_CP025268.1, 3950368, 3951888, -, 1521, 191247; cds-WP_001311244.1, NZ_CP025268.1, 3952241, 3952339, +, 99, 1; cds-WP _001387183.1, NZ_CP025268.1, 3952426, 3952476, +, 51, 5; cds-CXP41_RS20575, NZ_CP025268.1, 3952479, 3954123, +, 1645, 35755; cds-WP _000983255.1, NZ_CP025268.1, 3954120, 3954383, +, 264, 46571; cds-WP _000208520.1, NZ_CP025268.1, 3954403, 3955332, +, 930, 586; cds-WP_001127399.1, NZ_CP025268.1, 3955397, 3957247, +, 1851, 6; cds-WP_000785596.1, NZ_CP025268.1, 3957250, 3958794, +, 1545, 9; cds-WP _000365791.1, NZ_CP025268.1, 3958846, 3959739, -, 894, 5; cds-WP _000024939.1, NZ_CP025268.1, 3959889, 3961364, +, 1476, 136198; cds-WP _001140251.1, NZ_CP025268.1, 3961451, 3961732, -, 282, 1933; cds-CXP41_RS20615, NZ_CP025268.1, 3961931, 3962379, -, 449, 0; cds-WP_101348693.1, NZ_CP025268.1, 3962596, 3964617, +, 2022, 57897; cds-WP_101348694.1, NZ_CP025268.1, 3964664, 3966148, -, 1485, 1326354; cds-WP _000047499.1, NZ_CP025268.1, 3966284, 3967549, -, 1266, 444515; cds-WP _001280776.1, NZ_CP025268.1, 3967680, 3968009, +, 330, 82772; cds-WP _001295255.1, NZ_CP025268.1, 3968150, 3968251, +, 102, 99997; cds-WP_001054527.1, NZ_CP025268.1, 3968336, 3969595, +, 1260, 3609513; cds-WP_001050960.1, NZ_CP025268.1, 3969835, 3970938, +, 1104,
459326; cds-WP_101348695.1, NZ_CP025268.1, 3970950, 3972014, +, 1065, 881019; cds-WP _001340422.1, NZ_CP025268.1, 3972053, 3973183, +, 1131, 190405; cds-WP_000006621.1, NZ_CP025268.1, 3973180, 3974442, +, 1263, 461537; cds-WP _001226601.1, NZ_CP025268.1, 3974442, 3975509, +, 1068, 802708; cds-WP _000676056.1, NZ_CP025268.1, 3975528, 3976409, +, 882, 1592; cds-WP_001145183.1, NZ_CP025268.1, 3976387, 3977061, +, 675, 59260; cds-WP _000612043.1, NZ_CP025268.1, 3977066, 3978196, +, 1131, 511768; cds-WP_101348696.1, NZ_CP025268.1, 3978198, 3979448, +, 1251, 62950; cds-WP_000217234.1, NZ_CP025268.1, 3979445, 3980524, +, 1080, 242435; cds-WP _000055129.1, NZ_CP025268.1, 3980521, 3981873, +, 1353, 8305; cds-WP _001064040.1, NZ_CP025268.1, 3981876, 3982616, +, 741, 1; cds-CXP41_RS20720, NZ_CP025268.1, 3982807, 3984194, +, 1388, 124912; cds-WP _000941517.1, NZ_CP025268.1, 3984880, 3986115, +, 1236, 234957; cds-WP _000395863.1, NZ_CP025268.1, 3986274, 3987929, -, 1656, 1830; cds-WP _000921791.1, NZ_CP025268.1, 3988608, 3989804, -, 1197, 40427; cds-WP _000138997.1, NZ_CP025268.1, 3989807, 3990988, -, 1182, 298164; cds-WP _000026046.1, NZ_CP025268.1, 3991010, 3991750, -, 741, 135080; cds-WP _001338644.1, NZ_CP025268.1, 3991747, 3992688, -, 942, 48; cds-WP_101348697.1, NZ_CP025268.1, 3993075, 3995621, +, 2547, 1121125; cds-WP_000999947.1, NZ_CP025268.1, 3995661, 3995981, -, 321, 0; cds-WP_000799889.1, NZ_CP025268.1, 3996444, 3996647, +, 204, 461; cds-WP _001160654.1, NZ_CP025268.1, 3996684, 3997508, +, 825, 95123; cds-WP_000812796.1, NZ_CP025268.1, 3997505, 3998212, +, 708, 54573; cds-WP_000130691.1, NZ_CP025268.1, 3998209, 3999105, +, 897, 350117; cds-WP _001213584.1, NZ_CP025268.1, 3999105, 3999821, +, 717, 9434; cds-WP _000383406.1, NZ_CP025268.1, 3999905, 4002067, +, 2163, 104358; cds-WP_000951133.1, NZ_CP025268.1, 4002214, 4002978, -, 765, 268; cds-WP_211180520.1, NZ_CP025268.1, 4003140, 4003196, +, 57, 69288; cds-WP _000947159.1, NZ_CP025268.1, 4003348, 4004298, +, 951, 159779; cds-WP_000032581.1, NZ_CP025268.1, 4004341, 4004721, -, 381, 15; cds-WP_000955197.1, NZ_CP025268.1, 4004735, 4005151, -, 417, 0; cds-WP _000339104.1, NZ_CP025268.1, 4005210, 4006100, -, 891, 104828; cds-WP _001277142.1, NZ_CP025268.1, 4006152, 4006619, -, 468, 0; cds-WP_001259700.1, NZ_CP025268.1, 4006784, 4007653, +, 870, 86202; cds-WP _000035581.1, NZ_CP025268.1, 4007786, 4009615, +, 1830, 214996; cds-WP_000928824.1, NZ_CP025268.1, 4009679, 4010299, +, 621, 21520; cds-WP _000171710.1, NZ_CP025268.1, 4010361, 4010981, -, 621, 27806; cds-WP_000487654.1, NZ_CP025268.1, 4011092, 4012114, +, 1023, 49; cds-WP_000285362.1, NZ_CP025268.1, 4012122, 4012922, +, 801, 1017; cds-WP _001196238.1, NZ_CP025268.1, 4012998, 4013897, +, 900, 297; cds-WP _000573621.1, NZ_CP025268.1, 4013785, 4014738, -, 954, 0; cds-WP _000153907.1, NZ_CP025268.1, 4014975, 4017236, +, 2262, 33; cds-WP_001336442.1, NZ_CP025268.1, 4017276, 4018091, -, 816, 1; cds-WP_000045177.1, NZ_CP025268.1, 4018353, 4019114, +, 762, 3285; cds-WP _000347714.1, NZ_CP025268.1, 4019255, 4020682, +, 1428, 76900; cds-WP_000227958.1, NZ_CP025268.1, 4020777, 4021532, +, 756, 164300; cds-WP_001295259.1, NZ_CP025268.1, 4021546, 4022151, +, 606, 585342; cds-WP _000187530.1, NZ_CP025268.1, 4022148, 4023788, +, 1641, 708483; cds-WP_001295260.1, NZ_CP025268.1, 4023867, 4024136, +, 270, 19144; cds-WP_000459594.1, NZ_CP025268.1, 4024140, 4024655, +, 516, 508380; cds-WP_000109943.1, NZ_CP025268.1, 4024658, 4025434, +, 777, 47609; cds-WP _000459630.1, NZ_CP025268.1, 4025476, 4026258, +, 783, 8270; cds-WP_001192396.1, NZ_CP025268.1, 4026255, 4026743, -, 489, 16696; cds-WP_000339804.1, NZ_CP025268.1, 4026910, 4028403, +, 1494, 205198; cds-WP_000209826.1, NZ_CP025268.1, 4028449, 4029150, +, 702, 186952; cds-WP _000438725.1, NZ_CP025268.1, 4029531, 4030694, -, 1164, 227494; cds-WP_000965936.1, NZ_CP025268.1, 4030704, 4032893, -, 2190, 1831; cds-WP _000444561.1, NZ_CP025268.1, 4033083, 4034414, +, 1332, 219173; cds-WP _001308167.1, NZ_CP025268.1, 4034414, 4035028, +, 615, 74896; cds-WP _000545677.1, NZ_CP025268.1, 4035067, 4036518, +, 1452, 233528; cds-WP_000853959.1, NZ_CP025268.1, 4036530, 4037075, +, 546, 11744; cds-WP _000907622.1, NZ_CP025268.1, 4042828, 4043355, -, 528, 4; cds-WP_001052181.1, NZ_CP025268.1, 4043337, 4043921, -, 585, 4718; cds-WP_001295263.1, NZ_CP025268.1, 4043991, 4044260, +, 270, 17; cds-WP_001065497.1, NZ_CP025268.1, 4044337, 4045323, +, 987, 53485; cds-WP _000725337.1, NZ_CP025268.1, 4045340, 4045966, +, 627, 69008; cds-WP _001333507.1, NZ_CP025268.1, 4046121, 4047551, +, 1431, 1235; cds-WP _001311257.1, NZ_CP025268.1, 4047592, 4048524, -, 933, 404; cds-WP _000250006.1, NZ_CP025268.1, 4048888, 4051674, +, 2787, 94328; cds-WP _000183349.1, NZ_CP025268.1, 4052055, 4052687, -, 633, 36449; cds-WP_001295266.1, NZ_CP025268.1, 4053269, 4053778, +, 510, 18; cds-WP_000116090.1, NZ_CP025268.1, 4053967, 4055340, +, 1374, 50126; cds-WP _000893994.1, NZ_CP025268.1, 4055569, 4055679, -, 111, 5276; cds-WP_001188777.1, NZ_CP025268.1, 4055791, 4057200, -, 1410, 289713; cds-WP_000190577.1, NZ_CP025268.1, 4057212, 4058261, -, 1050, 46250; cds-WP_001271717.1, NZ_CP025268.1, 4058547, 4059956, -, 1410, 985980; cds-WP _000570668.1, NZ_CP025268.1, 4060329, 4062152, +, 1824, 2320178; cds-WP_000829798.1, NZ_CP025268.1, 4062369, 4063079, +, 711, 0; cds-WP _000256396.1, NZ_CP025268.1, 4063087, 4064067, +, 981, 0; cds-WP _000956313.1, NZ_CP025268.1, 4064169, 4065434, +, 1266, 0; cds-WP_000723465.1, NZ_CP025268.1, 4065525, 4066217, -, 693, 0; cds-WP_001295267.1, NZ_CP025268.1, 4066285, 4067688, -, 1404, 0; cds-WP _000018380.1, NZ_CP025268.1, 4067731, 4069116, -, 1386, 0; cds-WP_000380843.1, NZ_CP025268.1, 4069162, 4071198, -, 2037, 1107; cds-WP _001295268.1, NZ_CP025268.1, 4071397, 4072299, -, 903, 0; cds-WP _000870916.1, NZ_CP025268.1, 4072437, 4073678, -, 1242, 0; cds-WP _001046453.1, NZ_CP025268.1, 4073695, 4074573, -, 879, 2538; cds-WP_000718893.1, NZ_CP025268.1, 4074597, 4075493, -, 897, 0; cds-WP _000621656.1, NZ_CP025268.1, 4075661, 4076557, +, 897, 134; cds-WP _000059678.1, NZ_CP025268.1, 4076591, 4077376, +, 786, 4529; cds-WP_001295269.1, NZ_CP025268.1, 4077475, 4078074, +, 600,1; cds-WP_000920762.1, NZ_CP025268.1, 4078068, 4078940, +, 873, 8726; cds-WP _000560983.1, NZ_CP025268.1, 4078937, 4079374, +, 438, 3257; cds-WP_001297068.1, NZ_CP025268.1, 4079419, 4080360, +, 942, 71516; cds-WP_001295676.1, NZ_CP025268.1, 4081213, 4081431, +, 219, 0; cds-WP_001086388.1, NZ_CP025268.1, 4081649, 4081891, +, 243, 0; cds-WP_000027703.1, NZ_CP025268.1, 4082221, 4083150, -, 930, 322886; cds-WP_000829013.1, NZ_CP025268.1, 4083147, 4083782, -, 636, 166370; cds-WP_000331377.1, NZ_CP025268.1, 4083779, 4084681, -, 903, 1152679; cds-WP _010723259.1, NZ_CP025268.1, 4084694, 4087744, -, 3051, 6889307; cds-WP _000753617.1, NZ_CP025268.1, 4087938, 4088771, +, 834, 66; cds-WP_001295677.1, NZ_CP025268.1, 4088924, 4089979, +, 1056, 1; cds-WP_000931299.1, NZ_CP025268.1, 4090029, 4091777, -, 1749, 0; cds-WP_001019484.1, NZ_CP025268.1, 4091777, 4092847, -, 1071, 0; cds-WP _000446023.1, NZ_CP025268.1, 4092837, 4094288, -, 1452, 0; cds-WP _000729595.1, NZ_CP025268.1, 4094299, 4094745, -, 447, 0; cds-WP_000619493.1, NZ_CP025268.1, 4095046, 4095360, -, 315, 0; cds-WP _001179745.1, NZ_CP025268.1, 4095370, 4096194, -, 825, 0; cds-WP_001311268.1, NZ_CP025268.1, 4096645, 4097904, -, 1260, 213; cds-WP _000144073.1, NZ_CP025268.1, 4097901, 4099370, -, 1470, 0; cds-WP _000217137.1, NZ_CP025268.1, 4099658, 4100494, +, 837, 0; cds-WP_001336056.1, NZ_CP025268.1, 4100568, 4101416, +, 849, 2; cds-WP_000063526.1, NZ_CP025268.1, 4101413, 4102447, -, 1035, 1; cds-WP _000122641.1, NZ_CP025268.1, 4102732, 4103352, +, 621, 44783; cds-WP _001166063.1, NZ_CP025268.1, 4103612, 4104595, +, 984, 256; cds-WP _001270260.1, NZ_CP025268.1, 4104744, 4105418, +, 675, 32455; cds-WP _000580417.1, NZ_CP025268.1, 4105524, 4106897, -, 1374, 231365; cds-WP _001033722.1, NZ_CP025268.1, 4106894, 4107592, -, 699, 64674; cds-WP_001223800.1, NZ_CP025268.1, 4107742, 4108242, +, 501, 62564; cds-WP_001076742.1, NZ_CP025268.1, 4108391, 4109293, +, 903, 3; cds-WP _000591795.1, NZ_CP025268.1, 4109474, 4110436, +, 963, 157830; cds-WP _001045689.1, NZ_CP025268.1, 4110756, 4111745, +, 990, 5; cds-WP_001326656.1, NZ_CP025268.1, 4111852, 4112607, +, 756, 277; cds-WP_001216325.1, NZ_CP025268.1, 4112662, 4113429, -, 768, 16296; cds-WP_000802216.1, NZ_CP025268.1, 4113537, 4114136, -, 600, 0; cds-WP_000155257.1, NZ_CP025268.1, 4114237, 4114677, +, 441, 0; cds-WP_000655989.1, NZ_CP025268.1, 4114889, 4115188, +, 300, 158; cds-WP _000323556.1, NZ_CP025268.1, 4115215, 4115643, +, 429, 118532; cds-WP_000796332.1, NZ_CP025268.1, 4115648, 4116394, -, 747, 9; cds-WP_001250644.1, NZ_CP025268.1, 4116491, 4117501, -, 1011, 52444; cds-WP_101348698.1, NZ_CP025268.1, 4117636, 4119144, -, 1509, 77; cds-WP_000084268.1, NZ_CP025268.1, 4119167, 4120012, -, 846, 6133; cds-WP _001296623.1, NZ_CP025268.1, 4120437, 4120682, +, 246, 1648; cds-WP_000872908.1, NZ_CP025268.1, 4120767, 4121252, -, 486, 76969; cds-WP_000139496.1, NZ_CP025268.1, 4121345, 4122271, -, 927, 59632; cds-WP _001293341.1, NZ_CP025268.1, 4122338, 4123669, -, 1332, 937425; cds-WP_000208242.1, NZ_CP025268.1, 4123679, 4124209, -, 531, 289081; cds-WP_000068828.1, NZ_CP025268.1, 4124302, 4125261, -, 960, 175443; cds-WP_000644904.1, NZ_CP025268.1, 4125353, 4126378, -, 1026, 100086; cds-WP _001301269.1, NZ_CP025268.1, 4126534, 4128732, -, 2199, 764921; cds-WP _000710769.1, NZ_CP025268.1, 4128935, 4129147, +, 213, 60050; cds-WP _000797353.1, NZ_CP025268.1, 4129208, 4129816, -, 609, 48; cds-WP_000852812.1, NZ_CP025268.1, 4130000, 4130317, -, 318, 586; cds-WP _001295694.1, NZ_CP025268.1, 4130594, 4131754, +, 1161, 1; cds-WP_000110772.1, NZ_CP025268.1, 4131757, 4134189, +, 2433, 242835; cds-WP_000007523.1, NZ_CP025268.1, 4134538, 4135428, +, 891, 48; cds-WP _001295695.1, NZ_CP025268.1, 4135757, 4137937, +, 2181, 17216; cds-WP _001271242.1, NZ_CP025268.1, 4138030, 4138935, +, 906, 0; cds-WP_000647891.1, NZ_CP025268.1, 4138962, 4139579, -, 618, 0; cds-WP_000374004.1, NZ_CP025268.1, 4139854, 4140957, -, 1104, 0; cds-WP_000424840.1, NZ_CP025268.1, 4140968, 4141630, -, 663, 0; cds-WP_001174077.1, NZ_CP025268.1, 4141642, 4144143, -, 2502, 0; cds-WP_001004446.1, NZ_CP025268.1, 4144452, 4145531, +, 1080, 0; cds-WP_000161265.1, NZ_CP025268.1, 4145546, 4145866, +, 321, 0; cds-WP_000184811.1, NZ_CP025268.1, 4145917, 4148214, +, 2298, 44; cds-WP_000204105.1, NZ_CP025268.1, 4148180, 4149058, +, 879, 0; cds-WP_000323846.1, NZ_CP025268.1, 4149060, 4149401, +, 342, 0; cds-WP_101348699.1, NZ_CP025268.1, 4149388, 4150239, -, 852, 5; cds-WP_101348700.1, NZ_CP025268.1, 4150454, 4152187, -, 1734, 272899; cds-WP _001005586.1, NZ_CP025268.1, 4152369, 4155020, -, 2652, 85224; cds-WP _001298964.1, NZ_CP025268.1, 4155618, 4156769, -, 1152, 16687; cds-WP _000935370.1, NZ_CP025268.1, 4156923, 4157927, +, 1005, 8810; cds-WP_001302318.1, NZ_CP025268.1, 4157935, 4158711, +, 777, 17214; cds-WP_001230087.1, NZ_CP025268.1, 4158772, 4160145, +, 1374, 1; cds-WP_101348701.1, NZ_CP025268.1, 4160412, 4161329, +, 918, 16; cds-WP _001120810.1, NZ_CP025268.1, 4161312, 4162712, -, 1401, 2241; cds-WP _001309117.1, NZ_CP025268.1, 4163043, 4163693, +, 651, 42079; cds-WP_000806411.1, NZ_CP025268.1, 4163693, 4164052, +, 360, 27441; cds-WP_000187022.1, NZ_CP025268.1, 4164092, 4165192, -, 1101, 445044; cds-WP_000591359.1, NZ_CP025268.1, 4165561, 4167405, +, 1845, 106792; cds-WP_000201820.1, NZ_CP025268.1, 4167350, 4168207, +, 858, 6285; cds-WP_001016699.1, NZ_CP025268.1, 4173979, 4175007, +, 1029, 7481; cds-WP _000654630.1, NZ_CP025268.1, 4175004, 4175969, +, 966, 40101; cds-WP_000023081.1, NZ_CP025268.1, 4175998, 4176948, -, 951, 165948; cds-WP _000031784.1, NZ_CP025268.1, 4177866, 4179050, +, 1185, 9189359; cds-WP _001275702.1, NZ_CP025268.1, 4179280, 4179663, +, 384, 644556; cds-WP _001287516.1, NZ_CP025268.1, 4179665, 4180210, +, 546, 157940; cds-WP _001085926.1, NZ_CP025268.1, 4180369, 4180797, +, 429, 3020157; cds-WP _001096684.1, NZ_CP025268.1, 4180801, 4181505, +, 705, 10979757; cds-WP_001207201.1, NZ_CP025268.1, 4181918, 4182415, +, 498, 16356929; cds-WP _000028878.1, NZ_CP025268.1, 4182482, 4182847, +, 366, 7794591; cds-WP _000263098.1, NZ_CP025268.1, 4183167, 4187195, +, 4029, 17562917; cds-WP _000653944.1, NZ_CP025268.1, 4187272, 4191495, +, 4224, 18484796; cds-WP _001307500.1, NZ_CP025268.1,
4191708, 4192247, +, 540, 18627; cds-WP_000847447.1, NZ_CP025268.1, 4192657, 4193790, -, 1134, 135825; cds-WP_000944104.1, NZ_CP025268.1, 4193787, 4194557, -, 771, 43844; cds-WP_001166226.1, NZ_CP025268.1, 4194559, 4194759, -, 201, 0; cds-WP_000999737.1, NZ_CP025268.1, 4194743, 4195498, -, 756, 0; cds-WP_000284615.1, NZ_CP025268.1, 4195491, 4196126, -, 636, 0; cds-WP_001276926.1, NZ_CP025268.1, 4196126, 4198021, -, 1896, 198; cds-WP_000934302.1, NZ_CP025268.1, 4198254, 4198730, -, 477, 42; cds-WP_000373940.1, NZ_CP025268.1, 4198825, 4199598, +, 774, 44; cds-WP_000137657.1, NZ_CP025268.1, 4199638, 4200702, +, 1065, 13411; cds-WP_000362388.1, NZ_CP025268.1, 4200712, 4201383, +, 672, 68; cds-WP_000940106.1, NZ_CP025268.1, 4201426, 4202016, +, 591, 45282; cds-CXP41_RS21775, NZ_CP025268.1, 4202203, 4202475, +, 273, 20672; cds-WP_001084037.1, NZ_CP025268.1, 4202488, 4203183, +, 696, 20186; cds-WP_000828222.1, NZ_CP025268.1, 4203185, 4203610, -, 426, 7125; cds-WP_001211892.1, NZ_CP025268.1, 4203848, 4205245, +, 1398, 0; cds-WP_000148503.1, NZ_CP025268.1, 4205242, 4206567, +, 1326, 0; cds-WP_000866800.1, NZ_CP025268.1, 4206564, 4207853, -, 1290, 34; cds-WP_001187559.1, NZ_CP025268.1, 4207865, 4209454, -, 1590, 204981; cds-WP_001326558.1, NZ_CP025268.1, 4215156, 4215539, +, 384, 35837; cds-WP_000237004.1, NZ_CP025268.1, 4215602, 4216045, -, 444, 322093; cds-WP_001122779.1, NZ_CP025268.1, 4216202, 4217131, +, 930, 4100; cds-WP_000138905.1, NZ_CP025268.1, 4217400, 4219001, +, 1602, 35509; cds-WP_000857856.1, NZ_CP025268.1, 4219031, 4220335, +, 1305, 227; cds-WP_101348702.1, NZ_CP025268.1, 4220518, 4222254, +, 1737, 10; cds-WP_000632913.1, NZ_CP025268.1, 4222223, 4224409, -, 2187, 310; cds-WP_000226403.1, NZ_CP025268.1, 4224726, 4225550, -, 825, 1484; cds-WP_000096011.1, NZ_CP025268.1, 4225750, 4229433, +, 3684, 247; cds-WP_000956830.1, NZ_CP025268.1, 4229653, 4231284, +, 1632, 4817; cds-WP_000421763.1, NZ_CP025268.1, 4231375, 4232064, -, 690, 0; cds-WP_000936377.1, NZ_CP025268.1, 4232276, 4233148, +, 873, 815; cds-WP_001207634.1, NZ_CP025268.1, 4233281, 4233553, -, 273, 41; cds-WP_097756446.1, NZ_CP025268.1, 4233806, 4235155, -, 1350, 25878; cds-WP_000789986.1, NZ_CP025268.1, 4235680, 4237329, +, 1650, 746326; cds-WP_000757333.1, NZ_CP025268.1, 4237828, 4238070, +, 243, 0; cds-WP_001295278.1, NZ_CP025268.1, 4238184, 4238822, +, 639, 0; cds-WP_000595558.1, NZ_CP025268.1, 4238819, 4239556, +, 738, 0; cds-WP_000745776.1, NZ_CP025268.1, 4239556, 4241652, +, 2097, 0; cds-WP_001295279.1, NZ_CP025268.1, 4241699, 4241977, -, 279, 0; cds-WP_000202902.1, NZ_CP025268.1, 4242247, 4242657, +, 411, 0; cds-WP_001097274.1, NZ_CP025268.1, 4242701, 4244176, -, 1476, 0; cds-WP_001252058.1, NZ_CP025268.1, 4244548, 4245438, -, 891, 1; cds-WP_001297290.1, NZ_CP025268.1, 4245453, 4246997, -, 1545, 9; cds-WP_000695387.1, NZ_CP025268.1, 4247151, 4248341, -, 1191, 0; cds-WP_000179165.1, NZ_CP025268.1, 4248706, 4249821, +, 1116, 0; cds-WP_000973663.1, NZ_CP025268.1, 4249893, 4251233, +, 1341, 0; cds-WP_001326641.1, NZ_CP025268.1, 4251476, 4252396, +, 921, 3; cds-CXP41_RS22000, NZ_CP025268.1, 4252625, 4254205, +, 1581, 0; cds-WP_001326644.1, NZ_CP025268.1, 4254428, 4254925, +, 498, 3712; cds-WP_000455227.1, NZ_CP025268.1, 4254938, 4255810, +, 873, 18599; cds-WP_000017354.1, NZ_CP025268.1, 4255965, 4258388, -, 2424, 284739; cds-WP_000002907.1, NZ_CP025268.1, 4258559, 4258927, +, 369, 12636; cds-WP_000646078.1, NZ_CP025268.1, 4259037, 4259645, +, 609, 39652; cds-WP_001326646.1, NZ_CP025268.1, 4259718, 4261043, +, 1326, 147627; cds-WP_001030593.1, NZ_CP025268.1, 4261159, 4261368, +, 210, 10005; cds-WP_001295691.1, NZ_CP025268.1, 4261410, 4261925, -, 516, 10811; cds-CXP41_RS22055, NZ_CP025268.1, 4262243, 4263228, +, 986, 0; cds-WP_001298868.1, NZ_CP025268.1, 4263591, 4264628, +, 1038, 143325; cds-WP_000891404.1, NZ_CP025268.1, 4264762, 4265004, +, 243, 2339; cds-WP_000235508.1, NZ_CP025268.1, 4265170, 4266153, -, 984, 1429; cds-WP_000918363.1, NZ_CP025268.1, 4266236, 4267651, +, 1416, 301052; cds-WP_001147328.1, NZ_CP025268.1, 4267704, 4268783, +, 1080, 164949; cds-WP_000486985.1, NZ_CP025268.1, 4269036, 4270229, +, 1194, 3; cds-WP_001321562.1, NZ_CP025268.1, 4270732, 4270935, -, 204, 0; cds-WP_001226928.1, NZ_CP025268.1, 4271337, 4272050, +, 714, 0; cds-WP_000270375.1, NZ_CP025268.1, 4272161, 4272577, +, 417, 0; cds-WP_000155657.1, NZ_CP025268.1, 4272581, 4272937, +, 357, 0; cds-WP_000357740.1, NZ_CP025268.1, 4272972, 4275794, -, 2823, 317749; cds-WP_000168305.1, NZ_CP025268.1, 4276048, 4276584, +, 537, 41203; cds-WP_001295689.1, NZ_CP025268.1, 4276683, 4276964, -, 282, 8811; cds-WP_000019548.1, NZ_CP025268.1, 4277394, 4278980, +, 1587, 8; cds-WP_000019358.1, NZ_CP025268.1, 4278983, 4279306, -, 324, 2; cds-WP_000412428.1, NZ_CP025268.1, 4279392, 4279856, +, 465, 0; cds-WP_000106882.1, NZ_CP025268.1, 4280402, 4281751, +, 1350, 43933; cds-WP_000402210.1, NZ_CP025268.1, 4281903, 4283552, +, 1650, 15006; cds-WP_001270094.1, NZ_CP025268.1, 4283706, 4284998, -, 1293, 0; cds-WP_000832573.1, NZ_CP025268.1, 4285176, 4286825, -, 1650, 0; cds-WP_001014565.1, NZ_CP025268.1, 4286822, 4287136, -, 315, 0; cds-WP_000078239.1, NZ_CP025268.1, 4287336, 4289294, -, 1959, 618; cds-WP_000196875.1, NZ_CP025268.1, 4289687, 4291123, +, 1437, 0; cds-WP_001295391.1, NZ_CP025268.1, 4291168, 4291734, +, 567, 0; cds-WP_000220281.1, NZ_CP025268.1, 4291731, 4292402, +, 672, 0; cds-WP_000195171.1, NZ_CP025268.1, 4292399, 4293355, +, 957, 0; cds-WP_071524917.1, NZ_CP025268.1, 4293375, 4295093, +, 1719, 0; cds-WP_001032531.1, NZ_CP025268.1, 4295086, 4295469, +, 384, 0; cds-WP_000812896.1, NZ_CP025268.1, 4295466, 4296062, +, 597, 0; cds-WP_000789592.1, NZ_CP025268.1, 4296404, 4297717, +, 1314, 1316; cds-WP_000719886.1, NZ_CP025268.1, 4298361, 4299050, -, 690, 50184; cds-WP_101348703.1, NZ_CP025268.1, 4299144, 4301291, -, 2148, 0; cds-WP_000610601.1, NZ_CP025268.1, 4301489, 4302955, -, 1467, 0; cds-WP_101348704.1, NZ_CP025268.1, 4302952, 4305003, -, 2052, 0; cds-WP_000446378.1, NZ_CP025268.1, 4305003, 4306034, -, 1032, 0; cds-WP_001295389.1, NZ_CP025268.1, 4306053, 4306328, -, 276, 0; cds-WP_001066019.1, NZ_CP025268.1, 4306537, 4308522, -, 1986, 0; cds-WP_001171687.1, NZ_CP025268.1, 4308795, 4309724, -, 930, 0; cds-WP_001311314.1, NZ_CP025268.1, 4309708, 4310403, -, 696, 0; cds-WP_000507106.1, NZ_CP025268.1, 4310414, 4311394, -, 981, 0; cds-WP_000235257.1, NZ_CP025268.1, 4311373, 4312905, -, 1533, 0; cds-WP_001046187.1, NZ_CP025268.1, 4313032, 4313967, -, 936, 0; cds-WP_000083216.1, NZ_CP025268.1, 4314026, 4314916, -, 891, 0; cds-WP_000716794.1, NZ_CP025268.1, 4315275, 4315724, +, 450, 0; cds-WP_000819746.1, NZ_CP025268.1, 4315793, 4316122, +, 330, 0; cds-WP_000059908.1, NZ_CP025268.1, 4316269, 4317027, -, 759, 1; cds-WP_001110490.1, NZ_CP025268.1, 4317029, 4317463, -, 435, 0; cds-WP_000971886.1, NZ_CP025268.1, 4317450, 4318007, -, 558, 0; cds-WP_000586325.1, NZ_CP025268.1, 4318007, 4319143, -, 1137, 0; cds-WP_101348705.1, NZ_CP025268.1, 4319140, 4319820, -, 681, 0; cds-WP_001075514.1, NZ_CP025268.1, 4319931, 4320689, -, 759, 1; cds-WP_000002303.1, NZ_CP025268.1, 4320686, 4321531, -, 846, 0; cds-WP_001295388.1, NZ_CP025268.1, 4321524, 4322588, -, 1065, 0; cds-WP_000171628.1, NZ_CP025268.1, 4322588, 4323172, -, 585, 0; cds-WP_000542790.1, NZ_CP025268.1, 4323169, 4323621, -, 453, 0; cds-WP_001295387.1, NZ_CP025268.1, 4323622, 4324347, -, 726, 0; cds-CXP41_RS22375, NZ_CP025268.1, 4324368, 4325155, -, 788, 0; cds-WP_000992002.1, NZ_CP025268.1, 4325261, 4326277, -, 1017, 0; cds-WP_101348706.1, NZ_CP025268.1, 4326302, 4327090, -, 789, 0; cds-WP_001131339.1, NZ_CP025268.1, 4327223, 4327666, -, 444, 0; cds-WP_001300891.1, NZ_CP025268.1, 4328324, 4328659, -, 336, 1305; cds-WP_000288603.1, NZ_CP025268.1, 4329060, 4331288, +, 2229, 112300; cds-CXP41_RS22410, NZ_CP025268.1, 4331285, 4332164, +, 880, 5897; cds-WP_101348707.1, NZ_CP025268.1, 4332428, 4333930, +, 1503, 895790; cds-WP_000797657.1, NZ_CP025268.1, 4334042, 4334131, +, 90, 33076; cds-WP_001051092.1, NZ_CP025268.1, 4334107, 4335207, -, 1101, 101474; cds-WP_000697926.1, NZ_CP025268.1, 4335208, 4335876, -, 669, 7; cds-WP_000919792.1, NZ_CP025268.1, 4335873, 4337516, -, 1644, 0; cds-WP_000093154.1, NZ_CP025268.1, 4337620, 4338957, -, 1338, 0; cds-WP_001217060.1, NZ_CP025268.1, 4339094, 4339855, -, 762, 0; cds-WP_001381593.1, NZ_CP025268.1, 4340180, 4342447, -, 2268, 11197; cds-WP_001090758.1, NZ_CP025268.1, 4342646, 4343554, -, 909, 0; cds-WP_000986601.1, NZ_CP025268.1, 4343837, 4345192, +, 1356, 14; cds-WP_000028111.1, NZ_CP025268.1, 4345295, 4346716, +, 1422, 0; cds-WP_000198773.1, NZ_CP025268.1, 4346855, 4347484, -, 630, 0; cds-WP_101348708.1, NZ_CP025268.1, 4347606, 4349252, -, 1647, 5; cds-WP_000899522.1, NZ_CP025268.1, 4349330, 4350670, -, 1341, 0; cds-WP_000611288.1, NZ_CP025268.1, 4351241, 4351960, -, 720, 3; cds-WP_001216477.1, NZ_CP025268.1, 4351957, 4353588, -, 1632, 257482; cds-WP_000371704.1, NZ_CP025268.1, 4353769, 4353999, +, 231, 0; cds-WP_000405651.1, NZ_CP025268.1, 4354011, 4354283, +, 273, 151; cds-WP_000398619.1, NZ_CP025268.1, 4354510, 4354806, +, 297, 528; cds-WP_001173343.1, NZ_CP025268.1, 4354834, 4355007, +, 174, 305; cds-WP_001295074.1, NZ_CP025268.1, 4355126, 4356643, -, 1518, 524813; cds-WP_000856829.1, NZ_CP025268.1, 4356880, 4358337, -, 1458, 0; cds-WP_001295383.1, NZ_CP025268.1, 4358396, 4360543, -, 2148, 0; cds-WP_000092909.1, NZ_CP025268.1, 4360623, 4361957, -, 1335, 0; cds-WP_001187173.1, NZ_CP025268.1, 4362322, 4363860, -, 1539, 9375; cds-WP_001188520.1, NZ_CP025268.1, 4364659, 4365234, -, 576, 293; cds-WP_000068922.1, NZ_CP025268.1, 4365271, 4366968, -, 1698, 103; cds-WP_000883400.1, NZ_CP025268.1, 4366944, 4367282, -, 339, 359; cds-WP_000961959.1, NZ_CP025268.1, 4367398, 4368699, -, 1302, 49108; cds-WP_000069437.1, NZ_CP025268.1, 4368817, 4370253, -, 1437, 214; cds-WP_001267448.1, NZ_CP025268.1, 4370590, 4371066, +, 477, 0; cds-WP_000015837.1, NZ_CP025268.1, 4371082, 4372338, -, 1257, 46581; cds-WP_001026276.1, NZ_CP025268.1, 4372614, 4372907, +, 294, 74515; cds-WP_000729117.1, NZ_CP025268.1, 4372951, 4374597, +, 1647, 3333904; cds-WP_000558209.1, NZ_CP025268.1, 4374735, 4375088, +, 354, 0; cds-WP_001008040.1, NZ_CP025268.1, 4375291, 4376160, -, 870, 0; cds-WP_000940549.1, NZ_CP025268.1, 4376555, 4377583, -, 1029, 13765; cds-WP_000257278.1, NZ_CP025268.1, 4377625, 4378191, +, 567, 88357; cds-WP_000977757.1, NZ_CP025268.1, 4378243, 4378368, +, 126, 104613; cds-WP_000239596.1, NZ_CP025268.1, 4378479, 4378625, +, 147, 15126; cds-WP_000118482.1, NZ_CP025268.1, 4378801, 4379118, +, 318, 1233; cds-WP_001238378.1, NZ_CP025268.1, 4379115, 4379648, -, 534, 8; cds-WP_001336292.1, NZ_CP025268.1, 4379737, 4380870, -, 1134, 4266; cds-WP_000609663.1, NZ_CP025268.1, 4380933, 4381292, -, 360, 0; cds-WP_000208757.1, NZ_CP025268.1, 4381303, 4381698, -, 396, 0; cds-WP_000829498.1, NZ_CP025268.1, 4381709, 4382443, -, 735, 33; cds-WP_001192973.1, NZ_CP025268.1, 4382436, 4384244, -, 1809, 23; cds-WP_000004771.1, NZ_CP025268.1, 4384569, 4385546, +, 978, 80834; cds-WP_001336293.1, NZ_CP025268.1, 4385765, 4387267, +, 1503, 61256; cds-WP_000342867.1, NZ_CP025268.1, 4387319, 4387633, +, 315, 217210; cds-WP_001276180.1, NZ_CP025268.1, 4387630, 4387944, +, 315, 106616; cds-WP_001236847.1, NZ_CP025268.1, 4387973, 4391296, -, 3324, 118085; cds-WP_101348709.1, NZ_CP025268.1, 4391318, 4392286, -, 969, 1049361; cds-WP_000041964.1, NZ_CP025268.1, 4392383, 4393435, -, 1053, 383511; cds-WP_001295188.1, NZ_CP025268.1, 4393530, 4394075, +, 546, 86883; cds-WP_010723271.1, NZ_CP025268.1, 4394818, 4394871, +, 54, 178; cds-WP_001294219.1, NZ_CP025268.1, 4394854, 4395993, -, 1140, 70338; cds-WP_001295189.1, NZ_CP025268.1, 4395992, 4397539, +, 1548, 6; cds-WP_000981977.1, NZ_CP025268.1, 4397511, 4397972, +, 462, 0; cds-WP_000990333.1, NZ_CP025268.1, 4397991, 4399328, +, 1338, 288778; cds-WP_001122505.1, NZ_CP025268.1, 4399338, 4401185, +, 1848, 39527; cds-WP_001280345.1, NZ_CP025268.1, 4401178, 4402128, +, 951, 210169; cds-WP_001051883.1, NZ_CP025268.1, 4402214, 4402522, +, 309, 90721; cds-WP_000460362.1, NZ_CP025268.1, 4402598, 4403878, +, 1281, 902688; cds-WP_000312488.1, NZ_CP025268.1, 4403964, 4405223, +, 1260, 762357; cds-WP_001232412.1, NZ_CP025268.1, 4405226, 4406230, +, 1005, 660285; cds-WP_001089295.1, NZ_CP025268.1, 4406312, 4406509, +, 198, 11;
cds-WP_000527955.1, NZ_CP025268.1, 4406613, 4407911, +, 1299, 512206; cds-WP_001177639.1, NZ_CP025268.1, 4408116, 4408541, +, 426, 97852; cds-WP_000076332.1, NZ_CP025268.1, 4408580, 4411021, +, 2442, 482130; cds-WP_001293282.1, NZ_CP025268.1, 4411201, 4411932, +, 732, 18166; cds-WP_000220128.1, NZ_CP025268.1, 4412059, 4412460, +, 402, 24388; cds-WP_000511955.1, NZ_CP025268.1, 4412479, 4413177, +, 699, 0; cds-WP_000012550.1, NZ_CP025268.1, 4413228, 4413887, +, 660, 0; cds-WP_000547760.1, NZ_CP025268.1, 4413905, 4414303, +, 399, 0; cds-WP_000101658.1, NZ_CP025268.1, 4414313, 4414951, +, 639, 0; cds-WP_000943959.1, NZ_CP025268.1, 4414954, 4416117, +, 1164, 0; cds-WP_001350567.1, NZ_CP025268.1, 4416201, 4417826, +, 1626, 1; cds-WP_000811566.1, NZ_CP025268.1, 4417943, 4418218, -, 276, 0; cds-WP_000254633.1, NZ_CP025268.1, 4418367, 4418696, -, 330, 3572; cds-WP_000569707.1, NZ_CP025268.1, 4418878, 4419627, +, 750, 0; cds-WP_000133631.1, NZ_CP025268.1, 4419624, 4420379, -, 756, 2973; cds-WP_001295191.1, NZ_CP025268.1, 4420487, 4421551, -, 1065, 0; cds-WP_001350568.1, NZ_CP025268.1, 4421906, 4423303, +, 1398, 0; cds-WP_000218362.1, NZ_CP025268.1, 4423319, 4423624, +, 306, 0; cds-WP_000776517.1, NZ_CP025268.1, 4423634, 4424098, +, 465, 0; cds-WP_101348710.1, NZ_CP025268.1, 4424112, 4424762, +, 651, 0; cds-WP_000949502.1, NZ_CP025268.1, 4424772, 4425626, +, 855, 0; cds-WP_001170847.1, NZ_CP025268.1, 4425626, 4426312, +, 687, 0; cds-CXP41_RS22905, NZ_CP025268.1, 4426442, 4426716, -, 275, 0; cds-WP_001216676.1, NZ_CP025268.1, 4427043, 4427438, +, 396, 2428846; cds-WP_001315977.1, NZ_CP025268.1, 4427445, 4427759, +, 315, 4722802; cds-WP_000135199.1, NZ_CP025268.1, 4427764, 4427991, +, 228, 4344849; cds-WP_001196062.1, NZ_CP025268.1, 4428033, 4428482, +, 450, 2251752; cds-WP_000174714.1, NZ_CP025268.1, 4428553, 4429347, -, 795, 166290; cds-CXP41_RS22940, NZ_CP025268.1, 4429619, 4430020, +, 402, 0; cds-WP_001119478.1, NZ_CP025268.1, 4430004, 4430642, -, 639, 7; cds-WP_000211225.1, NZ_CP025268.1, 4430860, 4431480, +, 621, 938; cds-WP_000228346.1, NZ_CP025268.1, 4431789, 4433201, +, 1413, 8988; cds-WP_000331456.1, NZ_CP025268.1, 4433246, 4433908, -, 663, 4; cds-WP_001350569.1, NZ_CP025268.1, 4434016, 4434981, -, 966, 2; cds-WP_000560561.1, NZ_CP025268.1, 4435089, 4435949, -, 861, 25; cds-WP_000084622.1, NZ_CP025268.1, 4436038, 4436418, +, 381, 2; cds-WP_000589431.1, NZ_CP025268.1, 4436547, 4438490, -, 1944, 39160; cds-WP_000886919.1, NZ_CP025268.1, 4438680, 4439420, +, 741, 0; cds-WP_000937655.1, NZ_CP025268.1, 4439632, 4440570, +, 939, 69; cds-WP_000175279.1, NZ_CP025268.1, 4440633, 4441187, -, 555, 0; cds-WP_000689228.1, NZ_CP025268.1, 4441512, 4441718, +, 207, 2170; cds-WP_000935036.1, NZ_CP025268.1, 4441797, 4443140, -, 1344, 1492; cds-WP_101348711.1, NZ_CP025268.1, 4443463, 4444101, -, 639, 0; cds-WP_001269327.1, NZ_CP025268.1, 4444307, 4446040, +, 1734, 65055; cds-CXP41_RS23035, NZ_CP025268.1, 4446037, 4449815, +, 3779, 270027; cds-WP_001219160.1, NZ_CP025268.1, 4449818, 4450159, +, 342, 3488; cds-WP_001223208.1, NZ_CP025268.1, 4450371, 4450622, +, 252, 1164; cds-WP_000239577.1, NZ_CP025268.1, 4450616, 4450966, +, 351, 91; cds-WP_000055075.1, NZ_CP025268.1, 4451046, 4451576, -, 531, 62382; cds-WP_000265913.1, NZ_CP025268.1, 4451886, 4452842, +, 957, 68213; cds-WP_000205805.1, NZ_CP025268.1, 4452982, 4454484, +, 1503, 22; cds-WP_001313531.1, NZ_CP025268.1, 4454498, 4455520, +, 1023, 68; cds-WP_000596014.1, NZ_CP025268.1, 4455507, 4456502, +, 996, 5; cds-WP_000853753.1, NZ_CP025268.1, 4456535, 4457533, -, 999, 56; cds-WP_001219813.1, NZ_CP025268.1, 4457709, 4459082, +, 1374, 121451; cds-WP_000166270.1, NZ_CP025268.1, 4459238, 4459789, -, 552, 26242; cds-WP_001162171.1, NZ_CP025268.1, 4459883, 4461235, +, 1353, 646940; cds-WP_023649327.1, NZ_CP025268.1, 4461414, 4461779, +, 366, 596; cds-WP_001106233.1, NZ_CP025268.1, 4461824, 4462288, -, 465, 1; cds-WP_000187778.1, NZ_CP025268.1, 4462446, 4464584, -, 2139, 46173; cds-WP_001336303.1, NZ_CP025268.1, 4464978, 4466633, -, 1656, 141016; cds-WP_001407733.1, NZ_CP025268.1, 4466683, 4468104, -, 1422, 1; cds-WP_001181307.1, NZ_CP025268.1, 4468223, 4469170, -, 948, 78; cds-WP_001387276.1, NZ_CP025268.1, 4469355, 4469408, +, 54, 9463; cds-WP_000471889.1, NZ_CP025268.1, 4469549, 4472245, +, 2697, 1200541; cds-WP_000047539.1, NZ_CP025268.1, 4472451, 4472837, -, 387, 706; cds-WP_000148581.1, NZ_CP025268.1, 4472910, 4473371, -, 462, 129; cds-WP_000013046.1, NZ_CP025268.1, 4473384, 4474319, -, 936, 599; cds-WP_001296693.1, NZ_CP025268.1, 4474323, 4474457, -, 135, 0; cds-WP_000230281.1, NZ_CP025268.1, 4474738, 4475133, -, 396, 3272; cds-WP_000500727.1, NZ_CP025268.1, 4475264, 4475977, -, 714, 0; cds-CXP41_RS23175, NZ_CP025268.1, 4476058, 4476642, +, 585, 2; cds-WP_000583469.1, NZ_CP025268.1, 4476787, 4477239, +, 453, 30; cds-CXP41_RS23185, NZ_CP025268.1, 4477362, 4479177, +, 1816, 45; cds-WP_000012931.1, NZ_CP025268.1, 4479233, 4480237, -, 1005, 30; cds-WP_000002953.1, NZ_CP025268.1, 4480399, 4480815, +, 417, 15127; cds-WP_001059402.1, NZ_CP025268.1, 4480960, 4481463, -, 504, 40697; cds-WP_000079634.1, NZ_CP025268.1, 4481656, 4482852, +, 1197, 25632; cds-WP_000416392.1, NZ_CP025268.1, 4482908, 4485763, -, 2856, 1759757; cds-WP_000786400.1, NZ_CP025268.1, 4485763, 4486206, -, 444, 127753; cds-WP_000397144.1, NZ_CP025268.1, 4486366, 4487877, -, 1512, 216254; cds-WP_211180521.1, NZ_CP025268.1, 4488031, 4488081, +, 51, 194; cds-WP_000584114.1, NZ_CP025268.1, 4488144, 4489244, +, 1101, 10626; cds-WP_001295681.1, NZ_CP025268.1, 4489244, 4490326, +, 1083, 74728; cds-WP_001294573.1, NZ_CP025268.1, 4490487, 4491989, -, 1503, 1872; cds-WP_001309159.1, NZ_CP025268.1, 4492056, 4493054, -, 999, 16681; cds-WP_001128335.1, NZ_CP025268.1, 4493121, 4494440, -, 1320,6236; cds-WP_000998695.1, NZ_CP025268.1, 4494502, 4495266, -, 765, 0; cds-WP_001197411.1, NZ_CP025268.1, 4495290, 4496321, -, 1032, 0; cds-WP_000896738.1, NZ_CP025268.1, 4496538, 4497101, +, 564, 0; cds-WP_001309160.1, NZ_CP025268.1, 4497105, 4498124, -, 1020, 0; cds-CXP41_RS23280, NZ_CP025268.1, 4498590, 4499855, +, 1266, 109719; cds-WP_088895425.1-6, NZ_CP025268.1;NZ_CP025268.1, 4500187;4500502, 4500502;4501415, +;+, 1229, 52084; cds-CXP41_RS23290, NZ_CP025268.1, 4501508, 4502688, -, 1181, 21330; cds-CXP41_RS23305, NZ_CP025268.1, 4503275, 4503504, +, 230, 0; cds-WP_000483767.1, NZ_CP025268.1, 4504018, 4505364, -, 1347, 0; cds-WP_000179691.1, NZ_CP025268.1, 4505973, 4507190, +, 1218, 0; cds-WP_000611568.1, NZ_CP025268.1, 4507202, 4508320, +, 1119, 10; cds-WP_000594405.1, NZ_CP025268.1, 4508363, 4508488, +, 126, 0; cds-WP_000254999.1, NZ_CP025268.1, 4508541, 4508798, -, 258, 0; cds-CXP41_RS24310, NZ_CP025268.1, 4508788, 4509024, +, 237, 0; cds-CXP41_RS23345, NZ_CP025268.1, 4509112, 4509369, +, 258, 1; cds-WP_001254932.1-2, NZ_CP025268.1, 4509381, 4510532, -, 1152, 666; cds-CXP41_RS23355, NZ_CP025268.1, 4510597, 4510857, -, 261, 2; cds-CXP41_RS23360, NZ_CP025268.1, 4510924, 4511708, +, 785, 3; cds-WP_000177057.1, NZ_CP025268.1, 4511791, 4512048, +, 258, 0; cds-CXP41_RS23370, NZ_CP025268.1, 4512605, 4513372, -, 768, 15980; cds-WP_000684852.1, NZ_CP025268.1, 4513373, 4514329, -, 957, 127807; cds-WP_000125187.1, NZ_CP025268.1, 4514326, 4515324, -, 999, 4; cds-WP_000879155.1, NZ_CP025268.1, 4515321, 4516223, -, 903, 15316; cds-WP_101348713.1, NZ_CP025268.1, 4516268, 4518592, -, 2325, 47408; cds-WP_001068910.1, NZ_CP025268.1, 4518679, 4519632, -, 954, 126240; cds-WP_001283626.1, NZ_CP025268.1, 4519629, 4520150, -, 522, 10099; cds-WP_211180522.1, NZ_CP025268.1, 4520287, 4520355, +, 69, 0; cds-CXP41_RS23410, NZ_CP025268.1, 4520442, 4521139, +, 698, 40454; cds-WP_000373366.1, NZ_CP025268.1, 4521253, 4522239, -, 987, 2171; cds-WP_001128363.1, NZ_CP025268.1, 4522586, 4523935, -, 1350, 5; cds-WP_000116326.1, NZ_CP025268.1, 4524042, 4526009, -, 1968, 668; cds-WP_000714563.1, NZ_CP025268.1, 4526020, 4526925, -, 906, 0; cds-WP_000251798.1, NZ_CP025268.1, 4526930, 4527718, -, 789, 0; cds-WP_000082780.1, NZ_CP025268.1, 4528021, 4528803, -, 783, 0; cds-WP_000600622.1, NZ_CP025268.1, 4528820, 4529452, -, 633, 0; cds-WP_101348714.1, NZ_CP025268.1, 4529464, 4529895, -, 432, 0; cds-WP_000118626.1, NZ_CP025268.1, 4530026, 4530832, -, 807, 43725; cds-WP_000460843.1, NZ_CP025268.1, 4530845, 4532158, -, 1314, 1; cds-WP_000722973.1, NZ_CP025268.1, 4532170, 4532448, -, 279, 0; cds-WP_000010829.1, NZ_CP025268.1, 4532445, 4533566, -, 1122, 0; cds-CXP41_RS24410, NZ_CP025268.1, 4533811, 4533927, -, 117, 0; cds-CXP41_RS23480, NZ_CP025268.1, 4533965, 4534183, -, 219, 0; cds-WP_000354251.1, NZ_CP025268.1, 4534352, 4535098, -, 747, 0; cds-WP_001309181.1, NZ_CP025268.1, 4535154, 4535699, -, 546, 0; cds-WP_001054376.1, NZ_CP025268.1, 4535711, 4535968, -, 258, 0; cds-CXP41_RS23505, NZ_CP025268.1, 4536345, 4536590, -, 246, 0; cds-CXP41_RS23515, NZ_CP025268.1, 4536706, 4537946, +, 1241, 0; cds-WP_000991438.1, NZ_CP025268.1, 4538529, 4539509, -, 981, 0; cds-WP_001309184.1, NZ_CP025268.1, 4539574, 4540680, -, 1107, 0; cds-WP_001295734.1, NZ_CP025268.1, 4540700, 4541416, -, 717, 0; cds-WP_032162589.1, NZ_CP025268.1, 4542907, 4543473, +, 567, 10952; cds-WP_000044711.1, NZ_CP025268.1, 4543951, 4544547, +, 597, 1; cds-WP_000695564.1, NZ_CP025268.1, 4545029, 4545577, +, 549, 0; cds-WP_000824114.1, NZ_CP025268.1, 4545642, 4546181, +, 540, 0; cds-WP_000066547.1, NZ_CP025268.1, 4546218, 4546943, +, 726, 0; cds-WP_000121005.1, NZ_CP025268.1, 4547010, 4549646, +, 2637, 9; cds-WP_001244827.1, NZ_CP025268.1, 4549656, 4550186, +, 531, 0; cds-WP_000870592.1, NZ_CP025268.1, 4550199, 4550702, +, 504, 0; cds-WP_000832247.1, NZ_CP025268.1, 4550722, 4551624, +, 903, 0; cds-WP_000558251.1, NZ_CP025268.1, 4551867, 4553210, -, 1344, 0; cds-WP_000438562.1, NZ_CP025268.1, 4553550, 4554734, +, 1185, 354234; cds-WP_000208205.1, NZ_CP025268.1, 4554815, 4556275, +, 1461, 395; cds-WP_000833679.1, NZ_CP025268.1, 4556490, 4557263, +, 774, 2; cds-WP_000062552.1, NZ_CP025268.1, 4557404, 4558234, -, 831, 0; cds-WP_120795393.1, NZ_CP025268.1, 4558827, 4558910, +, 84, 0; cds-WP_001309187.1, NZ_CP025268.1, 4558907, 4559299, +, 393, 6868; cds-WP_000340740.1, NZ_CP025268.1, 4559292, 4560203, -, 912, 5871; cds-WP_000568432.1, NZ_CP025268.1, 4560268, 4561440, -, 1173, 33; cds-WP_000211971.1, NZ_CP025268.1, 4561453, 4561914, -, 462, 0; cds-WP_001295597.1, NZ_CP025268.1, 4561911, 4562594, -, 684, 45090; cds-WP_001151855.1, NZ_CP025268.1, 4562844, 4563398, +, 555, 0; cds-WP_001141220.1, NZ_CP025268.1, 4563411, 4564589, -, 1179, 0; cds-WP_001350572.1, NZ_CP025268.1, 4564657, 4565517, -, 861, 0; cds-WP_000657675.1, NZ_CP025268.1, 4565582, 4565839, -, 258, 0; cds-WP_000331745.1, NZ_CP025268.1, 4565836, 4566603, -, 768, 0; cds-WP_101348716.1, NZ_CP025268.1, 4566613, 4567764, -, 1152, 0; cds-WP_001037419.1, NZ_CP025268.1, 4567880, 4569160, -, 1281, 16424; cds-WP_001137036.1, NZ_CP025268.1, 4569201, 4570433, -, 1233, 30213; cds-CXP41_RS24325, NZ_CP025268.1, 4570912, 4571832, +, 921, 0; cds-WP_000199300.1, NZ_CP025268.1, 4572076, 4573488, -, 1413, 55426; cds-WP_000394280.1, NZ_CP025268.1, 4573665, 4573829, +, 165, 0; cds-CXP41_RS23730, NZ_CP025268.1, 4574328, 4575836, +, 1509, 8144; cds-CXP41_RS23735, NZ_CP025268.1, 4575833, 4578769, +, 2937, 22335; cds-WP_000437621.1, NZ_CP025268.1, 4578826, 4579872, -, 1047, 47608; cds-WP_000443951.1, NZ_CP025268.1, 4579872, 4581251, -, 1380, 124702; cds-WP_000132601.1, NZ_CP025268.1, 4581413, 4581754, -, 342, 0; cds-WP_001272447.1, NZ_CP025268.1, 4581982, 4583376, -, 1395, 61694; cds-WP_001063204.1, NZ_CP025268.1, 4583373, 4584962, -, 1590, 548219; cds-WP_000981388.1, NZ_CP025268.1, 4585163, 4588675, -, 3513, 30496; cds-WP_000217931.1, NZ_CP025268.1, 4588863, 4589777, +, 915, 13; cds-WP_001312515.1, NZ_CP025268.1, 4589823, 4590779, -, 957, 388; cds-WP_000467859.1, NZ_CP025268.1, 4590790, 4590993, -, 204, 169; cds-WP_001299714.1, NZ_CP025268.1, 4591043, 4593193, -, 2151, 200203; cds-WP_000919536.1, NZ_CP025268.1, 4593571, 4595226, +, 1656, 207925; cds-WP_000410113.1, NZ_CP025268.1, 4595275, 4596636, -, 1362, 540260; cds-WP_000091570.1, NZ_CP025268.1, 4596851, 4597765, -, 915, 0; cds-WP_000106030.1, NZ_CP025268.1, 4597904, 4598926, +, 1023, 249; cds-WP_001292679.1, NZ_CP025268.1, 4599064, 4601355, -, 2292, 448880; cds-WP_001308243.1, NZ_CP025268.1, 4601609, 4602103, -, 495, 6173; cds-WP_000799911.1, NZ_CP025268.1, 4602152, 4602889, -, 738, 70; cds-WP_000098818.1,
NZ_CP025268.1, 4602892, 4603431, -, 540, 26; cds-WP_000538191.1, NZ_CP025268.1, 4603538, 4604011, -, 474, 0; cds-WP_001338213.1, NZ_CP025268.1, 4604002, 4604772, -771, 0; cds-WP_000936635.1, NZ_CP025268.1, 4605391, 4606116, +, 726, 0; cds-WP_101348717.1, NZ_CP025268.1, 4606074, 4606751, +, 678, 10596; cds-WP_000331618.1, NZ_CP025268.1, 4606789, 4607577, -, 789, 7845; cds-WP_001393508.1, NZ_CP025268.1, 4607718, 4607954, +, 237, 70402; cds-WP_001272330.1, NZ_CP025268.1, 4608584, 4609615, -, 1032, 60867; cds-WP_000204012.1, NZ_CP025268.1, 4609718, 4610131, +, 414, 9474; cds-WP_001092461.1, NZ_CP025268.1, 4610100, 4610546, +, 447, 3952; cds-WP_000870710.1, NZ_CP025268.1, 4610561, 4611238, +, 678, 123988; cds-WP_000175940.1, NZ_CP025268.1, 4611330, 4612919, +, 1590, 494020; cds-WP_001295748.1, NZ_CP025268.1, 4613312, 4613917, +, 606, 28; cds-WP_000490275.1, NZ_CP025268.1, 4614044, 4614205, +, 162, 8; cds-WP_001299799.1, NZ_CP025268.1, 4614327, 4615400, +, 1074, 7; cds-WP_000563040.1, NZ_CP025268.1, 4615397, 4616176, +, 780, 0; cds-WP_001088415.1, NZ_CP025268.1, 4616596, 4617459, -, 864, 0; cds-WP_001143253.1, NZ_CP025268.1, 4617431, 4618981, -, 1551, 6; cds-WP_001298497.1, NZ_CP025268.1, 4619239, 4620018, +, 780, 22; cds-WP_101348718.1, NZ_CP025268.1, 4620145, 4621458, +, 1314, 283472; cds-WP_101348719.1, NZ_CP025268.1, 4621510, 4622733, +, 1224, 395661; cds-WP_000224877.1, NZ_CP025268.1, 4622790, 4623509, +, 720, 663269; cds-WP_001293115.1, NZ_CP025268.1, 4623676, 4625007, +, 1332, 249081; cds-WP_000105884.1, NZ_CP025268.1, 4625008, 4626024, -, 1017, 194; cds-WP_000124615.1, NZ_CP025268.1, 4626052, 4626696, -, 645, 21710; cds-WP_001132955.1, NZ_CP025268.1, 4626802, 4627770, +, 969, 16; cds-WP_001029687.1, NZ_CP025268.1, 4627819, 4629201, +, 1383, 20693; cds-WP_000093814.1, NZ_CP025268.1, 4629222, 4630454, +, 1233, 193503; cds-WP_000007444.1, NZ_CP025268.1, 4630488, 4630655, +, 168, 45; cds-WP_000046749.1, NZ_CP025268.1, 4630762, 4632429, -, 1668, 731111; cds-WP_000409451.1, NZ_CP025268.1, 4632640, 4634577, +, 1938, 25666; cds-WP_000068679.1, NZ_CP025268.1, 4634667, 4634993, +, 327, 3406; cds-WP_001338221.1, NZ_CP025268.1, 4635140, 4635652, -, 513, 457; cds-WP_000942344.1, NZ_CP025268.1, 4635704, 4636351, +, 648, 46735; cds-WP_000371666.1, NZ_CP025268.1, 4636348, 4637217, -, 870, 35267; cds-WP_000875487.1, NZ_CP025268.1, 4637428, 4637901, +, 474, 123; cds-WP_001188654.1, NZ_CP025268.1, 4637914, 4638603, +, 690, 3; cds-WP_001219577.1, NZ_CP025268.1, 4638603, 4640027, +, 1425, 44533; cds-WP_000920294.1, NZ_CP025268.1, 4640085, 4641437, +, 1353, 177380; cds-WP_001194358.1, NZ_CP025268.1, 4641497, 4642213, -, 717, 204297; cds-WP_001303782.1, NZ_CP025268.1, 4642309, 4642449, +, 141, 895; cds-WP_001223132.1, NZ_CP025268.1, 4642849, 4643535, +, 687, 17;

TABLE 9

Counts table of the small (12k) K. pneumoniae library when analyzed in bulk. Table organized as Geneid, Chr, Start, End, Strand, Length, K.pneumoniae_small; Geneid, Chr, Start, End, Strand, Length, K.pneumoniae_small; etc
cds-AF52_RS22895, NZ_KK737546.1, 1, 1202, +, 1202, 4; cds-AF52_RS22890, NZ_KK737546.1, 1257, 1415, -, 159, 212; cds-AF52_RS26590, NZ_KK737546.1, 6165, 6338, -, 174, 717; cds-AF52_RS26600, NZ_KK737546.1, 8440, 8543, -, 104, 173; cds-AF52_RS0122900, NZ_KK737546.1, 8577, 10166, -, 1590, 0; cds-WP_014228879.1, NZ_KK737546.1, 10308, 10652, -, 345, 0; cds-WP_032429382.1, NZ_KK737546.1, 10695, 10889, -, 195, 0; cds-AF52_RS0122905, NZ_KK737546.1, 11334, 11537, +, 204, 10; cds-AF52_RS26610, NZ_KK737546.1, 11581, 11765, +, 185, 208; cds-AF52_RS22870, NZ_KK737546.1, 12344, 12603, +, 260, 1028; cds-WP_040234937.1, NZ_KK737546.1, 13181, 13495, +, 315, 81216; cds-WP_032429384.1, NZ_KK737546.1, 13777, 14166, -, 390, 5608; cds-WP_072001558.1, NZ_KK737546.1, 14276, 14503, +, 228, 1137; cds-WP_032429385.1, NZ_KK737546.1, 14506, 15060, +, 555, 0; cds-WP_072001559.1, NZ_KK737546.1, 15105, 16124, +, 1020, 0; cds-WP_032429698.1, NZ_KK737546.1, 16117, 16581, +, 465, 2; cds-WP_032429386.1, NZ_KK737546.1, 16595, 17035, +, 441, 0; cds-WP_013815099.1, NZ_KK737546.1, 17339, 18307, -, 969, 1975; cds-WP_004147997.1, NZ_KK737546.1, 18343, 18546, -, 204, 14; cds-WP_139042930.1, NZ_KK737546.1, 19315, 19434, -, 120, 3; cds-WP_016160648.1, NZ_KK737546.1, 20352, 20567, +, 216, 0; cds-WP_032429387.1, NZ_KK737546.1, 20567, 21064, +, 498, 0; cds-WP_016160650.1, NZ_KK737546.1, 21061, 21411, +, 351, 0; cds-WP_050484272.1, NZ_KK737546.1, 22361, 22786, +, 426, 38; cds-WP_013815099.1-2, NZ_KK737546.1, 22812, 23780, -, 969, 7706; cds-WP_023289182.1, NZ_KK737546.1, 23903, 24127, -, 225, 997; cds-WP_162924227.1, NZ_KK737546.1, 24306, 24470, +, 165, 0; cds-AF52_RS24930, NZ_KK737546.1, 24454, 24816, +, 363, 0; cds-WP_023289183.1, NZ_KK737546.1, 24768, 25091, +, 324, 0; cds-WP_023289184.1, NZ_KK737546.1, 25088, 25519, +, 432, 0; cds-WP_012542168.1, NZ_KK737546.1, 25767, 26201, +, 435, 0; cds-WP_021462603.1, NZ_KK737546.1, 26201, 27922, +, 1722, 0; cds-WP_017898992.1, NZ_KK737546.1, 27916, 28095, +, 180, 4; cds-WP_012542166.1, NZ_KK737546.1, 28095, 29354, +, 1260, 0; cds-WP_032429388.1, NZ_KK737546.1, 29391, 30311, +, 921, 0; cds-WP_032429389.1, NZ_KK737546.1, 30389, 31675, +, 1287, 0; cds-WP_014907814.1, NZ_KK737546.1, 31734, 31994, +, 261, 0; cds-WP_020317538.1, NZ_KK737546.1, 31975, 32292, +, 318, 0; cds-WP_014228910.1, NZ_KK737546.1, 32289, 32627, +, 339, 0; cds-WP_032429390.1, NZ_KK737546.1, 32608, 32997, +, 390, 0; cds-WP_017898997.1, NZ_KK737546.1, 32994, 33395, +, 402, 0; cds-WP_014228913.1, NZ_KK737546.1, 33427, 33888, +, 462, 19; cds-WP_014228914.1, NZ_KK737546.1, 33946, 34311, +, 366, 0; cds-WP_032429392.1, NZ_KK737546.1, 34544, 37879, +, 3336, 0; cds-WP_014228916.1, NZ_KK737546.1, 37879, 38217, +, 339, 0; cds-WP_032429393.1, NZ_KK737546.1, 38214, 38969, +, 756, 0; cds-WP_023317958.1, NZ_KK737546.1, 38971, 39681, +, 711, 0; cds-WP_032429394.1, NZ_KK737546.1, 39714, 40061, +, 348, 0; cds-WP_023159867.1, NZ_KK737546.1, 40113, 40706, +, 594, 0; cds-AF52_RS0122945, NZ_KK737546.1, 40769, 53314, +, 12546, 79; cds-WP_032429396.1, NZ_KK737546.1, 53376, 54872, +, 1497, 5; cds-WP_004892953.1, NZ_KK737546.1, 55027, 55179, +,153,188; cds-WP_023279886.1, NZ_KK737546.1, 55452, 56165, -, 714, 0; cds-WP_004190914.1, NZ_KK737546.1, 56162, 56554, -, 393, 0; cds-WP_004150797.1, NZ_KK737546.1, 56547, 56870, -, 324, 0; cds-AF52_RS0122950, NZ_KK737546.1, 56993, 57166, -, 174, 3; cds-WP_004140530.1, NZ_KK737546.1, 57320, 57547, -, 228, 2; cds-WP_021462619.1, NZ_KK737546.1, 57660, 58853, -, 1194, 0; cds-WP_004150795.1, NZ_KK737546.1, 59476, 59661, +, 186, 0; cds-WP_004148027.1, NZ_KK737546.1, 59752, 60246, +, 495, 0; cds-WP_004140514.1, NZ_KK737546.1, 60273, 60779, +, 507, 0; cds-WP_013815099.1-3, NZ_KK737546.1, 60849, 61817, +, 969, 6093; cds-WP_032429398.1, NZ_KK737546.1, 61874, 62749, +, 876, 19; cds-WP_004140511.1, NZ_KK737546.1, 62805, 64211, +, 1407, 3; cds-WP_004183659.1, NZ_KK737546.1, 64208, 65218, +, 1011, 87; cds-WP_004140506.1, NZ_KK737546.1, 65334, 65531, -, 198, 0; cds-WP_004183660.1, NZ_KK737546.1, 66098, 66730, +, 633, 11; cds-WP_004140501.1, NZ_KK737546.1, 66770, 66949, +, 180, 0; cds-WP_004150790.1, NZ_KK737546.1, 67347, 68033, +, 687, 34940; cds-WP_004150789.1, NZ_KK737546.1, 68146, 68310, -, 165, 39; cds-WP_004140497.1, NZ_KK737546.1, 68344, 69852, -, 1509, 0; cds-WP_021313530.1, NZ_KK737546.1, 69973, 70863, +, 891, 1; cds-WP_004213090.1, NZ_KK737546.1, 70870, 72654, -, 1785, 10587; cds-WP_004140494.1, NZ_KK737546.1, 72728, 73936, -, 1209, 37; cds-WP_004179363.1, NZ_KK737546.1, 74239, 75282, +, 1044, 22; cds-WP_004148038.1, NZ_KK737546.1, 75944, 76858, +, 915, 545; cds-WP_004150783.1, NZ_KK737546.1, 76948, 77586, +, 639, 16; cds-WP_004140489.1, NZ_KK737546.1, 77717, 77980, +, 264, 1132; cds-WP_004140488.1, NZ_KK737546.1, 78039, 78164, -, 126, 828; cds-WP_004213093.1, NZ_KK737546.1, 78282, 78356, -, 75, 1; cds-WP_004150782.1, NZ_KK737546.1, 78356, 78457, -, 102, 28; cds-WP_004176549.1, NZ_KK737546.1, 78515, 79528, -, 1014, 1112; cds-WP_004176548.1, NZ_KK737546.1, 79829, 80068, +, 240, 8031; cds-WP_014343000.1, NZ_KK737546.1, 80058, 80396, -, 339, 3; cds-WP_020802835.1, NZ_KK737546.1, 80401, 80910, -, 510, 3; cds-WP_021441569.1, NZ_KK737546.1, 81056, 81748, +, 693, 1811; cds-WP_020324105.1, NZ_KK737546.1, 81780, 82955, -, 1176, 3850; cds-WP_004140469.1, NZ_KK737546.1, 83063, 83857, +, 795, 3; cds-WP_002901080.1, NZ_KK737546.1, 83841, 84287, -, 447, 2; cds-WP_002901088.1, NZ_KK737546.1, 84404, 84904, -, 501, 8; cds-WP_072271772.1, NZ_KK737546.1, 85151, 86287, +, 1137, 0; cds-WP_002901096.1, NZ_KK737546.1, 86458, 86700, -, 243, 1; cds-WP_002901192.1, NZ_KK737546.1, 86971, 87390, +, 420, 0; cds-WP_004152360.1, NZ_KK737546.1, 87393, 88658, +, 1266, 0; cds-WP_021441571.1, NZ_KK737546.1, 88665, 89570, -, 906, 3272; cds-WP_004176542.1, NZ_KK737546.1, 89737, 90486, +, 750, 0; cds-WP_021441573.1, NZ_KK737546.1, 90483, 91700, -, 1218, 0; cds-WP_004179386.1, NZ_KK737546.1, 91876, 92757, +, 882, 23; cds-WP_004220356.1, NZ_KK737546.1, 92828, 92947, -, 120, 342; cds-WP_016946410.1, NZ_KK737546.1, 93015, 93326, +, 312, 0; cds-WP_032428825.1, NZ_KK737546.1, 93618, 94166, +, 549, 0; cds-WP_124053788.1, NZ_KK737546.1, 94180, 94551, +, 372, 0; cds-WP_004140429.1, NZ_KK737546.1, 94548, 95831, -, 1284, 20; cds-WP_002901234.1, NZ_KK737546.1, 95917, 97851, -, 1935, 4403; cds-WP_002901236.1, NZ_KK737546.1, 98268, 99014, +, 747, 59720; cds-WP_072143276.1, NZ_KK737546.1, 99145, 99960, +, 816, 12129; cds-WP_002901240.1, NZ_KK737546.1, 100017, 100901, -, 885, 124773; cds-WP_002901243.1, NZ_KK737546.1, 100976, 101971, -, 996, 484985; cds-WP_002901246.1, NZ_KK737546.1, 102310, 102723, +, 414, 8523; cds-WP_002901249.1, NZ_KK737546.1, 102767, 103045, +, 279, 483; cds-WP_002901254.1, NZ_KK737546.1, 103140, 103484, -, 345, 2002; cds-WP_002901255.1, NZ_KK737546.1, 103645, 104898, -, 1254, 11894; cds-WP_004140413.1, NZ_KK737546.1, 105070, 105711, -, 642, 24509; cds-WP_004176535.1, NZ_KK737546.1, 105721, 106740, -, 1020, 140153; cds-WP_004140411.1, NZ_KK737546.1, 106879, 108732, -, 1854, 532; cds-WP_004176531.1, NZ_KK737546.1, 108898, 109449, +, 552, 19; cds-WP_002901387.1, NZ_KK737546.1, 110185, 110931, -, 747, 11261; cds-WP_004176528.1, NZ_KK737546.1, 111157, 112200, +, 1044, 112; cds-WP_032428827.1, NZ_KK737546.1, 112205, 114151, +, 1947, 13156; cds-WP_004140406.1, NZ_KK737546.1, 114331, 115311, +, 981, 26; cds-WP_004176526.1, NZ_KK737546.1, 115355, 116698, -, 1344, 461; cds-WP_004183673.1, NZ_KK737546.1, 116881, 117291, -, 411, 74; cds-WP_002901398.1, NZ_KK737546.1, 117378, 118001, +, 624, 0; cds-WP_032429402.1, NZ_KK737546.1, 118053, 119360, -, 1308, 115; cds-WP_032428829.1, NZ_KK737546.1, 119454, 120086, -, 633, 0; cds-WP_004892845.1, NZ_KK737546.1, 120086, 121621, -, 1536, 0; cds-WP_004224226.1, NZ_KK737546.1, 121594, 122772, -, 1179, 0; cds-WP_004176519.1, NZ_KK737546.1, 122775, 123335, -, 561, 0; cds-WP_004151923.1, NZ_KK737546.1, 123332, 124042, -, 711, 0; cds-WP_021441584.1, NZ_KK737546.1, 124029, 124685, -, 657, 2; cds-WP_002901486.1, NZ_KK737546.1, 124899, 125705, -, 807, 13016; cds-WP_002901489.1, NZ_KK737546.1, 126144,127364, +, 1221, 19157; cds-WP_032429405.1, NZ_KK737546.1, 127361, 128395, +, 1035, 13524; cds-WP_004151922.1, NZ_KK737546.1, 128392, 129870, +, 1479, 34155;
cds-WP_004892826.1, NZ_KK737546.1, 129867, 131192, +, 1326, 717; cds-WP_021441588.1, NZ_KK737546.1, 131202, 132167, +, 966, 21; cds-WP_004217130.1, NZ_KK737546.1, 132511, 132996, +, 486, 32034; cds-WP_004176514.1, NZ_KK737546.1, 133084, 133947, -, 864, 0; cds-WP_032429406.1, NZ_KK737546.1, 134048, 134875, -, 828, 13161; cds-WP_002901544.1, NZ_KK737546.1, 135158, 135496, +, 339, 654; cds-WP_013815099.1-4, NZ_KK737546.1, 135868, 136836, +, 969, 7626; cds-WP_050484274.1, NZ_KK737546.1, 136880, 137161, +, 282, 10; cds-WP_014907779.1, NZ_KK737546.1, 137181, 137297, +, 117, 0; cds-WP_004148091.1, NZ_KK737546.1, 137309, 138667, +, 1359, 0; cds-WP_004183683.1, NZ_KK737546.1, 138721, 139068, +, 348, 0; cds-WP_002901552.1, NZ_KK737546.1, 139096, 139920, +, 825, 19; cds-WP_032429409.1, NZ_KK737546.1, 140031, 141377, +, 1347, 7628; cds-WP_004179430.1, NZ_KK737546.1, 141390, 142148, +, 759, 9; cds-WP_002901554.1, NZ_KK737546.1, 142206, 144464, -, 2259, 7358; cds-WP_004140343.1, NZ_KK737546.1, 144577, 144909, +, 333, 0; cds-WP_032429412.1, NZ_KK737546.1, 144969, 146360, -, 1392, 127; cds-WP_002901611.1, NZ_KK737546.1, 146496, 147086, -, 591, 0; cds-WP_004176511.1, NZ_KK737546.1, 147178, 147939, -, 762, 648; cds-WP_004140335.1, NZ_KK737546.1, 148089, 148757, -, 669, 7; cds-WP_002901627.1, NZ_KK737546.1, 148946, 149482, +, 537, 372; cds-WP_002901629.1, NZ_KK737546.1, 149512, 149910, -, 399, 13; cds-AF52_RS0123015, NZ_KK737546.1, 150010, 152194, -, 2185, 0; cds-WP_002901631.1, NZ_KK737546.1, 152466, 153005, -, 540, 15; cds-WP_002901632.1, NZ_KK737546.1, 153060, 153803, -, 744, 8777; cds-WP_032429413.1, NZ_KK737546.1, 153829, 154242, -, 414, 437; cds-WP_002901634.1, NZ_KK737546.1, 154512, 155150, +, 639, 0; cds-WP_032429414.1, NZ_KK737546.1, 155235, 155912, -, 678, 13723; cds-WP_032429415.1, NZ_KK737546.1, 155961, 158246, -, 2286, 0; cds-WP_021441592.1, NZ_KK737546.1, 158556, 159263, -, 708, 2; cds-WP_004151911.1, NZ_KK737546.1, 159382, 159582, +, 201, 0; cds-WP_032429416.1, NZ_KK737546.1, 159691, 160638, -, 948, 0; cds-WP_021441594.1, NZ_KK737546.1, 160713, 161171, +, 459, 0; cds-WP_004183698.1, NZ_KK737546.1, 161214, 162122, -, 909, 30; cds-WP_004210755.1, NZ_KK737546.1, 162251, 163561, +, 1311, 0; cds-WP_002901724.1, NZ_KK737546.1, 163576, 164664, +, 1089, 6; cds-WP_004179454.1, NZ_KK737546.1, 164667, 165926, +, 1260, 0; cds-WP_021441596.1, NZ_KK737546.1, 166145, 166954, -, 810, 2; cds-WP_032423750.1, NZ_KK737546.1, 166954, 168147, -, 1194, 23066; cds-WP_032429417.1, NZ_KK737546.1, 168157, 169515, -, 1359, 255; cds-WP_021441599.1, NZ_KK737546.1, 169519, 171114, -, 1596, 0; cds-WP_004176488.1, NZ_KK737546.1, 171114, 172676, -, 1563, 1034; cds-WP_002901735.1, NZ_KK737546.1, 172965, 173828, +, 864, 203; cds-WP_002901738.1, NZ_KK737546.1, 173845, 174465, +, 621, 3597; cds-WP_021441600.1, NZ_KK737546.1, 174487, 175395, -, 909, 0; cds-WP_004176482.1, NZ_KK737546.1, 175507, 176403, +, 897, 0; cds-WP_002901746.1, NZ_KK737546.1, 176685, 177587, +, 903, 991; cds-WP_002901749.1, NZ_KK737546.1, 177648, 178238, -, 591, 270; cds-WP_032429418.1, NZ_KK737546.1, 178235, 178996, -, 762, 21659; cds-WP_004148112.1, NZ_KK737546.1, 178991, 179206, +, 216, 1; cds-WP_002901758.1, NZ_KK737546.1, 179251, 180297, +, 1047, 13728; cds-WP_002901761.1, NZ_KK737546.1, 180345, 180596, -, 252, 54; cds-WP _002901763.1, NZ_KK737546.1, 181003, 183600, +, 2598, 87342; cds-WP_002901772.1, NZ_KK737546.1, 183946, 184920, +, 975, 17094; cds-WP_002901776.1, NZ_KK737546.1, 185166, 185333, +, 168, 5049; cds-WP_032429420.1, NZ_KK737546.1, 185722, 188400, +, 2679, 8958; cds-WP_032429421.1, NZ_KK737546.1, 188448, 189050, -, 603, 64; cds-WP_002901779.1, NZ_KK737546.1, 189214, 189981, +, 768, 855; cds-WP_002901780.1, NZ_KK737546.1, 190117, 190425, +, 309, 216; cds-WP_002901781.1, NZ_KK737546.1, 190432, 191601, +, 1170, 17; cds-WP_014907762.1, NZ_KK737546.1, 191793, 192530, +, 738, 2; cds-WP_004210732.1, NZ_KK737546.1, 192530, 192856, +, 327, 0; cds-WP_002901783.1, NZ_KK737546.1, 192988, 193206, -, 219, 87; cds-WP_002901785.1, NZ_KK737546.1, 193482, 194231, -, 750, 54; cds-WP_002901786.1, NZ_KK737546.1, 194303, 194482, -, 180, 615; cds-WP_002901787.1, NZ_KK737546.1, 194641, 196575, -, 1935, 27359; cds-WP_032421254.1, NZ_KK737546.1, 196657, 197814, -, 1158, 2; cds-WP_004140277.1, NZ_KK737546.1, 198005, 198793, -, 789, 51406; cds-WP_014907760.1, NZ_KK737546.1, 198992, 199534, -, 543, 15124; cds-WP_004151902.1, NZ_KK737546.1, 199782, 201161, +, 1380,8909; cds-WP_004140269.1, NZ_KK737546.1, 201206, 202015, -, 810, 4254; cds-WP_004140266.1, NZ_KK737546.1, 202017, 203009, -, 993, 83222; cds-WP_004151901.1, NZ_KK737546.1, 203009, 203899, -, 891, 3160; cds-WP_032429422.1, NZ_KK737546.1, 204046, 205263, +, 1218, 0; cds-WP_004892750.1, NZ_KK737546.1, 205484, 205723, -, 240, 0; cds-WP_016831904.1, NZ_KK737546.1, 205731, 206039, -, 309, 0; cds-WP_108918811.1, NZ_KK737546.1, 206036, 206677, -, 642, 0; cds-WP_016831906.1, NZ_KK737546.1, 206689, 206910, -, 222, 0; cds-WP_032426563.1, NZ_KK737546.1, 206907, 207434, -, 528, 0; cds-WP_008807811.1, NZ_KK737546.1, 207463, 208086, -, 624, 14; cds-WP_032429423.1, NZ_KK737546.1, 208083, 208829, -, 747, 0; cds-WP_008807813.1, NZ_KK737546.1, 208846, 209130, -, 285, 0; cds-WP_008807814.1, NZ_KK737546.1, 209211, 209417, -, 207, 1; cds-WP_161477215.1, NZ_KK737546.1, 209410, 209568, -, 159, 165; cds-WP_004223168.1, NZ_KK737546.1, 209854, 210057, +, 204, 1; cds-WP_008807815.1, NZ_KK737546.1, 210087, 210734, -, 648, 44865; cds-WP_008807816.1, NZ_KK737546.1, 210731, 211165, -, 435, 15398; cds-WP_024622727.1, NZ_KK737546.1, 211385, 212074, -, 690, 910; cds-WP_004178811.1, NZ_KK737546.1, 212179, 212412, +, 234, 0; cds-WP_004141720.1, NZ_KK737546.1, 212452, 212772, +, 321, 8; cds-WP_004151298.1, NZ_KK737546.1, 212859, 213005, +, 147, 0; cds-WP_073545955.1, NZ_KK737546.1, 213046, 213897, +, 852, 1; cds-WP_075209545.1, NZ_KK737546.1, 213902, 215317, +, 1416, 10; cds-WP_032429424.1, NZ_KK737546.1, 215317, 215619, +, 303, 0; cds-AF52_RS21920, NZ_KK737546.1, 215616, 215912, +, 297, 0; cds-WP_032429425.1, NZ_KK737546.1, 216035, 216298, +, 264, 0; cds-AF52_RS25000, NZ_KK737546.1, 216450, 216854, +, 405, 0; cds-WP_023286281.1, NZ_KK737546.1, 217078, 217299, +, 222, 0; cds-WP_032429427.1, NZ_KK737546.1, 217303, 218010, +, 708, 13; cds-WP_016831923.1, NZ_KK737546.1, 218000, 218224, +, 225, 0; cds-WP_032429428.1, NZ_KK737546.1, 218299, 218811, +, 513, 7; cds-WP_032429704.1, NZ_KK737546.1, 218879, 219136, +, 258, 3; cds-WP_032429429.1, NZ_KK737546.1, 219243, 219839, +, 597, 16; cds-WP_004146340.1, NZ_KK737546.1, 220048, 220338, +, 291, 3; cds-WP_004177081.1, NZ_KK737546.1, 220335, 220697, +, 363, 0; cds-WP_023279926.1, NZ_KK737546.1, 220694, 220834, +, 141, 0; cds-WP_032429430.1, NZ_KK737546.1, 220831, 221520, +, 690, 0; cds-WP_024940884.1, NZ_KK737546.1, 222173, 222472, +, 300, 0; cds-WP_008807831.1, NZ_KK737546.1, 222469, 223008, +, 540, 11023; cds-WP_004190674.1, NZ_KK737546.1, 223005, 223349, +, 345, 0; cds-WP_032429431.1, NZ_KK737546.1, 223346, 223621, +, 276, 0; cds-WP_004218558.1, NZ_KK737546.1, 224580, 224825, +, 246, 98; cds-WP_013815099.1-5, NZ_KK737546.1, 225397, 226365, -, 969, 6184; cds-WP_032429432.1, NZ_KK737546.1, 226754, 227758, +, 1005, 57; cds-WP_004190663.1, NZ_KK737546.1, 227736, 229043, +, 1308, 2027; cds-AF52_RS21820, NZ_KK737546.1, 229043, 229582, +, 540, 3806; cds-WP_170965341.1, NZ_KK737546.1, 229616, 229810, +, 195, 331; cds-AF52_RS26695, NZ_KK737546.1, 231136, 231250, +, 115, 163; cds-WP_187079184.1, NZ_KK737546.1, 231498, 232175, +, 678, 41493; cds-WP_032429434.1, NZ_KK737546.1, 232159, 233271, +, 1113, 15197; cds-WP_032429435.1, NZ_KK737546.1, 233356, 234141, +, 786, 28; cds-WP_032429436.1, NZ_KK737546.1, 234152, 235105, +, 954, 152229; cds-WP_142689607.1, NZ_KK737546.1, 235114, 235386, +, 273, 14109; cds-WP_004217348.1, NZ_KK737546.1, 235427, 235822, +, 396, 2460; cds-WP_004190649.1, NZ_KK737546.1, 235824, 236078, +, 255, 31591; cds-WP_004217344.1, NZ_KK737546.1, 236307, 236690, +, 384, 0; cds-WP_008807839.1, NZ_KK737546.1, 236692, 237243, +, 552, 0; cds-WP_004190640.1, NZ_KK737546.1, 237240, 237632, +, 393, 0; cds-WP_004217341.1, NZ_KK737546.1, 237656, 238828, +, 1173, 225738; cds-WP_004226994.1, NZ_KK737546.1, 238882, 239364, +, 483, 0; cds-WP_074185736.1, NZ_KK737546.1, 239502, 239696, +, 195, 0; cds-WP_032429437.1, NZ_KK737546.1, 240136, 240315, +, 180, 10065; cds-WP_032417044.1, NZ_KK737546.1, 240325, 240810, +, 486, 48355; cds-AF52_RS21755, NZ_KK737546.1, 240849, 241094, -, 246, 17803; cds-WP_013815099.1-6, NZ_KK737546.1, 241160, 242128, -, 969, 9202; cds-WP_032429438.1, NZ_KK737546.1, 242146, 242397, -, 252, 0; cds-WP_077254381.1, NZ_KK737546.1, 242561, 242902, +, 342, 4; cds-AF52_RS21745, NZ_KK737546.1, 243004, 244416, +, 1413, 6993; cds-WP_013815099.1-7, NZ_KK737546.1, 244471, 245439, -, 969, 4011; cds-AF52_RS21735, NZ_KK737546.1, 245474, 246952, +, 1479, 85; cds-WP_032429439.1, NZ_KK737546.1, 246952, 247425, +, 474, 0; cds-WP_032429440.1, NZ_KK737546.1, 247412, 247894, +, 483, 0; cds-WP_032429442.1, NZ_KK737546.1, 247904, 248284, +, 381, 0; cds-AF52_RS21715, NZ_KK737546.1, 248281, 251259, +, 2979, 50; cds-WP_013815099.1-8, NZ_KK737546.1, 251293, 252261, +, 969, 3512; cds-AF52_RS0123075, NZ_KK737546.1, 252965, 253330, -, 366, 0; cds-WP_032429443.1, NZ_KK737546.1, 253422, 253970, +, 549, 0; cds-WP_004179627.1, NZ_KK737546.1, 255020, 255439, +, 420, 430; cds-AF52_RS0123080, NZ_KK737546.1, 255441, 256706, +, 1266, 2; cds-WP_032429444.1, NZ_KK737546.1, 256748, 257389, +, 642, 93; cds-WP_016831214.1, NZ_KK737546.1, 257382, 258056, -, 675, 1; cds-WP_002901816.1, NZ_KK737546.1, 258261, 259226, -, 966, 26785; cds-WP_004140258.1, NZ_KK737546.1, 259223, 260866, -, 1644,8092; cds-WP_021441607.1, NZ_KK737546.1, 261200, 262108, -, 909, 0; cds-WP_019705465.1, NZ_KK737546.1, 262216, 263046, +, 831, 0; cds-WP_032429447.1, NZ_KK737546.1, 263025, 265334, -, 2310, 2422; cds-WP_002901900.1, NZ_KK737546.1, 265506, 266675, +, 1170, 0; cds-WP_021441611.1, NZ_KK737546.1, 266701, 268092, +, 1392, 34; cds-WP_004148137.1, NZ_KK737546.1, 268083, 269072, -, 990, 2; cds-WP_002901908.1, NZ_KK737546.1, 269225, 269893, +, 669, 10356; cds-WP_002901911.1, NZ_KK737546.1, 269949, 270173, +, 225, 35994; cds-WP_002901913.1, NZ_KK737546.1, 270173, 270532, +, 360, 15; cds-WP_002901915.1, NZ_KK737546.1, 270561, 270779, +, 219, 784; cds-WP_002901917.1, NZ_KK737546.1, 270882, 272279, +, 1398, 67; cds-WP_004140239.1, NZ_KK737546.1, 272276, 273337, +, 1062, 3; cds-WP_004888583.1, NZ_KK737546.1, 273427, 274326, -, 900, 12; cds-WP_002901977.1, NZ_KK737546.1, 274440, 275753, +, 1314, 0; cds-WP_002901979.1, NZ_KK737546.1, 275926, 277467, +, 1542, 91; cds-WP_032428836.1, NZ_KK737546.1, 277569, 278621, +, 1053, 671; cds-WP_004152131.1, NZ_KK737546.1, 278692, 279198, -, 507, 9283; cds-WP_032429452.1, NZ_KK737546.1, 279301, 280266, +, 966, 53; cds-WP_004152134.1, NZ_KK737546.1, 280263, 280970, -, 708, 3; cds-WP_004148146.1, NZ_KK737546.1, 281043, 281159, +, 117, 97; cds-WP_004176446.1, NZ_KK737546.1, 281156, 282772, +, 1617, 12506; cds-WP_021441615.1, NZ_KK737546.1, 282873, 283259, -, 387, 1048; cds-WP_004190572.1, NZ_KK737546.1, 283490, 284323, +, 834, 357; cds-WP_004152138.1, NZ_KK737546.1, 284448, 285332, -, 885, 6; cds-WP_004190569.1, NZ_KK737546.1, 285505, 286641, +, 1137, 0; cds-WP_023287551.1, NZ_KK737546.1, 286714, 287916, +, 1203, 0; cds-WP_004152141.1, NZ_KK737546.1, 287995, 288864, +, 870, 30762; cds-AF52_RS21545, NZ_KK737546.1, 289095, 289340, +, 246, 1; cds-WP_072001575.1, NZ_KK737546.1, 289412, 289558, -, 147, 17; cds-WP_013815099.1-9, NZ_KK737546.1, 289602, 290570, -, 969, 3218; cds-WP_187079185.1, NZ_KK737546.1, 290586, 291959, +, 1374, 0; cds-WP_002902422.1, NZ_KK737546.1, 292005, 292940, -, 936, 2545; cds-WP_004140161.1, NZ_KK737546.1, 293174, 293608, -, 435, 82; cds-WP_002902432.1, NZ_KK737546.1, 293690, 293902, -, 213, 0; cds-WP_004176397.1, NZ_KK737546.1, 294046, 295170, -, 1125, 0; cds-WP_021313170.1, NZ_KK737546.1, 295527, 299054, -, 3528, 28151; cds-WP_004140155.1, NZ_KK737546.1, 299417, 299680, +, 264, 0; cds-WP_032429459.1, NZ_KK737546.1, 299726, 300166, -, 441, 1; cds-WP_002902515.1, NZ_KK737546.1, 300289, 301278, -, 990, 62821; cds-WP_002902516.1, NZ_KK737546.1, 301426, 301926, -, 501, 0; cds-WP_023279029.1, NZ_KK737546.1, 302133, 304772, +, 2640, 182; cds-WP_004140145.1, NZ_KK737546.1, 304769, 304954, +, 186, 29; cds-WP_002902522.1, NZ_KK737546.1, 304962, 305285, +, 324, 1; cds-WP_004140140.1,
NZ_KK737546.1, 305286, 306188, -, 903, 143; cds-WP_032429462.1, NZ_KK737546.1, 306424, 307923, +, 1500, 22; cds-WP_032429463.1, NZ_KK737546.1, 308048, 308938, +, 891, 179; cds-WP_021440033.1, NZ_KK737546.1, 308982, 309965, +, 984, 34; cds-WP_021440034.1, NZ_KK737546.1, 309968, 312235, -, 2268, 8251; cds-WP _032429464.1, NZ_KK737546.1, 312712, 314757, -, 2046, 59991; cds-WP_032428268.1, NZ_KK737546.1, 315044, 315973, +, 930, 168104; cds-WP_032429465.1, NZ_KK737546.1, 315985, 316272, +, 288, 85592; cds-WP_020324632.1, NZ_KK737546.1, 316280, 317035, +, 756, 50114; cds-WP_021440037.1, NZ_KK737546.1, 317047, 317544, +, 498, 92127; cds-WP_002902772.1, NZ_KK737546.1, 317552, 318622, +, 1071, 80402; cds-WP_032429468.1, NZ_KK737546.1, 318619, 319386, +, 768, 6208; cds-WP_004140117.1, NZ_KK737546.1, 319389, 320177, +, 789, 11398; cds-WP_021440038.1, NZ_KK737546.1, 320178, 321602, +, 1425, 26687; cds-WP_002902876.1, NZ_KK737546.1, 321592, 322014, +, 423, 8960; cds-WP_014907683.1, NZ_KK737546.1, 322014, 323219, +, 1206, 3868; cds-WP_002902877.1, NZ_KK737546.1, 323246, 324562, +, 1317, 4263; cds-WP_004194367.1, NZ_KK737546.1, 324682, 325608, +, 927, 33362; cds-WP_002902883.1, NZ_KK737546.1, 325618, 326214, +, 597, 0; cds-WP_024940864.1, NZ_KK737546.1, 326223, 326633, +, 411, 1; cds-WP_002902889.1, NZ_KK737546.1, 326670, 327275, -, 606, 9233; cds-WP_032429470.1, NZ_KK737546.1, 327480, 331382, +, 3903, 7550; cds-WP_020324670.1, NZ_KK737546.1, 331443, 332117, -, 675, 39; cds-WP_021440039.1, NZ_KK737546.1, 332118, 333497, -, 1380, 64086; cds-WP_002902965.1, NZ_KK737546.1, 333494, 334579, -, 1086, 37323; cds-WP_002902967.1, NZ_KK737546.1, 334608, 336260, -, 1653, 648; cds-WP_002902969.1, NZ_KK737546.1, 336260, 336691, -, 432, 6; cds-WP_002902971.1, NZ_KK737546.1, 336799, 337746, -, 948, 0; cds-WP_032429472.1, NZ_KK737546.1, 337847, 338845, +, 999, 2963; cds-WP_004151574.1, NZ_KK737546.1, 339001, 340275, -, 1275, 678112; cds-WP_004224524.1, NZ_KK737546.1, 340488, 340634, -, 147, 6898; cds-WP_020802294.1, NZ_KK737546.1, 340804, 342009, +, 1206, 128906; cds-WP_004140076.1, NZ_KK737546.1, 342028, 343050, +, 1023, 61699; cds-WP_004183870.1, NZ_KK737546.1, 343041, 344507, +, 1467, 64759; cds-WP_004190366.1, NZ_KK737546.1, 344529, 345869, +, 1341, 14107; cds-WP_004176358.1, NZ_KK737546.1, 345880, 346875, +, 996, 3046; cds-WP_016947259.1, NZ_KK737546.1, 346885, 348315, +, 1431, 36098; cds-WP_021440044.1, NZ_KK737546.1, 348524, 349849, -, 1326, 348; cds-WP_020316474.1, NZ_KK737546.1, 350230, 351030, +, 801, 1; cds-WP_004140057.1, NZ_KK737546.1, 351092, 351229, -, 138, 2; cds-WP_004151568.1, NZ_KK737546.1, 351228, 352667, +, 1440, 79; cds-WP_002903227.1, NZ_KK737546.1, 352716, 353714, -, 999, 1803; cds-WP_002903230.1, NZ_KK737546.1, 353997, 354155, +, 159, 1; cds-WP_004224558.1, NZ_KK737546.1, 354190, 354582, -, 393, 0; cds-WP_002903233.1, NZ_KK737546.1, 354833, 355066, -, 234, 475; cds-WP_021440045.1, NZ_KK737546.1, 355063, 356271, -, 1209, 0; cds-WP_004179665.1, NZ_KK737546.1, 356375, 356728, -, 354, 0; cds-WP_004179667.1, NZ_KK737546.1, 356965, 357444, +, 480, 0; cds-WP_004151566.1, NZ_KK737546.1, 357514, 357717, -, 204, 0; cds-WP_032429476.1, NZ_KK737546.1, 358029, 359057, +, 1029, 0; cds-WP_032429478.1, NZ_KK737546.1, 359071, 360243, -, 1173, 18504; cds-WP_021440047.1, NZ_KK737546.1, 360295, 361887, -, 1593, 12134; cds-WP_021440048.1, NZ_KK737546.1, 362060, 363088, +, 1029, 850; cds-WP_004190339.1, NZ_KK737546.1, 363128, 364687, -, 1560, 33208; cds-WP_032429480.1, NZ_KK737546.1, 364774, 365952, -, 1179, 46643; cds-WP_032429482.1, NZ_KK737546.1, 366154, 367800, +, 1647, 376679; cds-WP_002903315.1, NZ_KK737546.1, 368140, 369540, +, 1401, 2457; cds-WP_021440051.1, NZ_KK737546.1, 369530, 370462, -, 933, 14; cds-WP_021440052.1, NZ_KK737546.1, 370538, 371839, -, 1302, 51278; cds-WP_032429484.1, NZ_KK737546.1, 372095, 372457, -, 363, 1307; cds-WP_002903377.1, NZ_KK737546.1, 372700, 373419, -, 720, 51; cds-WP_002903379.1, NZ_KK737546.1, 373548, 373883, +, 336, 53; cds-WP_004179681.1, NZ_KK737546.1, 373880, 374602, -, 723, 66; cds-WP_004183892.1, NZ_KK737546.1, 374639, 376021, -, 1383, 1; cds-WP_002903384.1, NZ_KK737546.1, 376199, 377149, -, 951, 39992; cds-WP_004140012.1, NZ_KK737546.1, 377146, 377328, -, 183, 347; cds-WP_032422490.1, NZ_KK737546.1, 377671, 379200, +, 1530, 30984; cds-WP_002903386.1, NZ_KK737546.1, 379211, 380599, +, 1389, 49553; cds-WP_004887037.1, NZ_KK737546.1, 380748, 380936, +, 189, 2; cds-WP _002903391.1, NZ_KK737546.1, 381046, 381996, -, 951, 28733; cds-WP_002903394.1, NZ_KK737546.1, 382135, 382887, -, 753, 23533; cds-WP_004224598.1, NZ_KK737546.1, 383082, 383597, -, 516, 0; cds-WP_002903398.1, NZ_KK737546.1, 383890, 384048, +, 159, 0; cds-AF52_RS21120, NZ_KK737546.1, 384081, 384533, -, 453, 0; cds-WP_023343160.1, NZ_KK737546.1, 384656, 384919, -, 264, 0; cds-WP_032429712.1, NZ_KK737546.1, 384927, 385493, -, 567, 0; cds-WP_072001561.1, NZ_KK737546.1, 385495, 386064, -, 570, 0; cds-WP_032429489.1, NZ_KK737546.1, 386061, 386354, -, 294, 0; cds-AF52_RS0123125, NZ_KK737546.1, 386354, 386536, -, 183, 0; cds-WP_032429490.1, NZ_KK737546.1, 386715, 386915, -, 201,0; cds-WP_032429491.1, NZ_KK737546.1, 386912, 387394, -, 483, 0; cds-AF52_RS26290, NZ_KK737546.1, 387508, 387846, -, 339, 0; cds-WP_032429493.1, NZ_KK737546.1, 388073, 388858, -, 786, 0; cds-WP_023343167.1, NZ_KK737546.1, 388851, 389051, -, 201, 0; cds-WP_032429494.1, NZ_KK737546.1, 389051, 389578, -, 528, 0; cds-WP_032429495.1, NZ_KK737546.1, 389714, 390544, -, 831, 8; cds-WP_032429496.1, NZ_KK737546.1, 390597, 390968, -, 372, 0; cds-WP_187079182.1, NZ_KK737546.1, 391972, 392158, +, 187, 209; cds-AF52_RS26730, NZ_KK737546.1, 393389, 393527, +, 139, 3720; cds-WP_032429497.1, NZ_KK737546.1, 393786, 394019, -, 234, 1; cds-WP_032429029.1, NZ_KK737546.1, 394208, 394867, -, 660, 16047; cds-WP_023343174.1, NZ_KK737546.1, 394955, 395155, +, 201, 0; cds-WP_032429498.1, NZ_KK737546.1, 395200, 395751, +, 552, 1395; cds-WP_023322342.1, NZ_KK737546.1, 395924, 396103, +, 180, 0; cds-AF52_RS21035, NZ_KK737546.1, 396093, 396482, +, 390, 0; cds-WP_013815099.1-10, NZ_KK737546.1, 396540, 397508, -, 969, 2680; cds-AF52_RS21025, NZ_KK737546.1, 397537, 398793, -, 1257, 318; cds-WP_004143402.1, NZ_KK737546.1, 399073, 399846, -, 774, 3; cds-WP_023301931.1, NZ_KK737546.1, 399893, 401398, -, 1506, 28629; cds-WP_021440130.1, NZ_KK737546.1, 401452, 402201, -, 750, 755; cds-WP_021440129.1, NZ_KK737546.1, 402565, 403365, +, 801, 33; cds-AF52_RS21000, NZ_KK737546.1, 403475, 404163, +, 689, 0; cds-WP_032429499.1, NZ_KK737546.1, 404175, 405119, +, 945, 0; cds-WP_004143414.1, NZ_KK737546.1, 405156, 406490, +, 1335, 8; cds-WP_004176179.1, NZ_KK737546.1, 406492, 406881, +, 390, 0; cds-WP_021440127.1, NZ_KK737546.1, 407130, 408686, +, 1557, 23; cds-WP_021440126.1, NZ_KK737546.1, 408683, 410275, +, 1593, 23; cds-WP_004190119.1, NZ_KK737546.1, 410287, 411021, +, 735, 8; cds-WP_004148393.1, NZ_KK737546.1, 411033, 412223, +, 1191, 18; cds-WP_004143425.1, NZ_KK737546.1, 412359, 413696, +, 1338, 116; cds-WP_004148392.1, NZ_KK737546.1, 413710, 414588, +, 879, 6; cds-WP_004176181.1, NZ_KK737546.1, 414665, 416035, +, 1371, 2760; cds-WP_021440125.1, NZ_KK737546.1, 416049, 417224, +, 1176, 2; cds-WP_002904834.1, NZ_KK737546.1, 417221, 418378, +, 1158, 11; cds-WP_002904832.1, NZ_KK737546.1, 418461, 419336, -, 876, 0; cds-WP_021440123.1, NZ_KK737546.1, 419448, 420332, -, 885, 1; cds-WP_002904827.1, NZ_KK737546.1, 420444, 421187, +, 744, 6; cds-WP_002904825.1, NZ_KK737546.1, 421193, 421891, -, 699, 4623; cds-WP_032417116.1, NZ_KK737546.1, 422159, 423691, +, 1533, 13; cds-WP_021440120.1, NZ_KK737546.1, 423703, 425031, +, 1329, 1; cds-WP_032417115.1, NZ_KK737546.1, 425777, 427222, +, 1446, 250262; cds-WP_021440118.1, NZ_KK737546.1, 427212, 427655, +, 444, 93061; cds-WP_002904816.1, NZ_KK737546.1, 428072, 428311, +, 240, 0; cds-WP_009308870.1, NZ_KK737546.1, 428336, 428476, +, 141, 125; cds-WP_004148380.1, NZ_KK737546.1, 428664, 429599, +, 936, 1; cds-WP_004143447.1, NZ_KK737546.1, 429633, 431288, -, 1656, 40727; cds-WP_002904810.1, NZ_KK737546.1, 431585, 431833, +, 249, 168; cds-WP_002904808.1, NZ_KK737546.1, 432123, 432341, +, 219, 9; cds-WP_004179825.1, NZ_KK737546.1, 432322, 433104, +, 783, 7507; cds-WP_004176193.1, NZ_KK737546.1, 433331, 433618, -, 288, 0; cds-WP_004143457.1, NZ_KK737546.1, 434062, 434625, +, 564, 0; cds-WP_004176194.1, NZ_KK737546.1, 434703, 435398, +, 696, 0; cds-WP_016831617.1, NZ_KK737546.1, 435414, 437915, +, 2502, 0; cds-WP_004176196.1, NZ_KK737546.1, 437903, 438907, +, 1005, 8; cds-WP_002904794.1, NZ_KK737546.1, 439049, 440341, +, 1293, 2071; cds-WP_004176197.1, NZ_KK737546.1, 440358, 441680, -, 1323, 1; cds-WP_032417111.1, NZ_KK737546.1, 441680, 441946, -, 267, 6; cds-WP_002904788.1, NZ_KK737546.1, 442211, 442537, -, 327, 0; cds-WP_002904787.1, NZ_KK737546.1, 442534, 443034, -, 501, 670; cds-WP_032429500.1, NZ_KK737546.1, 443012, 444391, -, 1380, 2; cds-WP_002904783.1, NZ_KK737546.1, 444737, 445891, +, 1155, 7184; cds-WP_032429501.1, NZ_KK737546.1, 445872, 446795, -, 924, 5978; cds-WP_021440114.1, NZ_KK737546.1, 446921, 447958, +, 1038, 92; cds-WP_032429502.1, NZ_KK737546.1, 447970, 448953, +, 984, 0; cds-WP_014907616.1, NZ_KK737546.1, 448930, 450168, -, 1239, 21; cds-WP_032429503.1, NZ_KK737546.1, 450274, 451218, +, 945, 8; cds-WP_032429504.1, NZ_KK737546.1, 451228, 451983, -, 756, 0; cds-WP_002904646.1, NZ_KK737546.1, 452160, 452471, -, 312, 0; cds-WP_002904643.1, NZ_KK737546.1, 452493, 452756, -, 264, 51; cds-WP_004176215.1, NZ_KK737546.1, 453039, 454067, +, 1029, 0; cds-WP_002904638.1, NZ_KK737546.1, 454091, 454897, +, 807, 2; cds-WP_021440110.1, NZ_KK737546.1, 454894, 455685, +, 792, 0; cds-WP_021440109.1, NZ_KK737546.1, 455682, 456341, +, 660, 819; cds-WP_021440108.1, NZ_KK737546.1, 456352, 457425, -, 1074, 5; cds-WP_004143508.1, NZ_KK737546.1, 457500, 458345, +, 846, 0; cds-WP_032417109.1, NZ_KK737546.1, 458342, 459499, -, 1158, 15325; cds-WP_032417108.1, NZ_KK737546.1, 459707, 460129, +, 423, 189; cds-WP_004176220.1, NZ_KK737546.1, 460285, 461472, +, 1188, 16; cds-WP_002904624.1, NZ_KK737546.1, 461701, 461991, +, 291, 8; cds-WP_004176221.1, NZ_KK737546.1, 462200, 462961, +, 762, 0; cds-WP_004143517.1, NZ_KK737546.1, 463122, 463442, +, 321, 0; cds-WP_032429505.1, NZ_KK737546.1, 463433, 463813, +, 381, 0; cds-WP_021440101.1, NZ_KK737546.1, 464024, 464470, -, 447, 24; cds-WP_004183995.1, NZ_KK737546.1, 464724, 466175, -, 1452, 43391; cds-WP_021440100.1, NZ_KK737546.1, 466289, 466819, -, 531, 15; cds-WP_004148352.1, NZ_KK737546.1, 466897, 468321, -, 1425, 7443; cds-WP_004190206.1, NZ_KK737546.1, 468436, 468804, -, 369, 0; cds-AF52_RS0123140, NZ_KK737546.1, 468804, 469730, -, 927, 58; cds-WP_021440098.1, NZ_KK737546.1, 469811, 471199, -, 1389, 0; cds-WP_002904495.1, NZ_KK737546.1, 471301, 472179, +, 879, 0; cds-WP_009308907.1, NZ_KK737546.1, 472133, 472615, -, 483, 0; cds-WP_021440097.1, NZ_KK737546.1, 472760, 473704, +, 945, 115; cds-WP_002904489.1, NZ_KK737546.1, 473719, 474480, -, 762, 31; cds-WP_016532445.1, NZ_KK737546.1, 474482, 475468, -, 987, 58; cds-WP_032429506.1, NZ_KK737546.1, 475461, 476696, -, 1236, 0; cds-WP_004179774.1, NZ_KK737546.1, 476871, 478019, +, 1149, 404; cds-WP_021440094.1, NZ_KK737546.1, 478034, 478918, +, 885, 14029; cds-WP_021440093.1, NZ_KK737546.1, 479004, 479954, +, 951, 18; cds-WP_004179768.1, NZ_KK737546.1, 480018, 481337, +, 1320, 4; cds-WP_004179766.1, NZ_KK737546.1, 481383, 482177, -, 795, 28; cds-WP_002904407.1, NZ_KK737546.1, 482379, 483578, +, 1200, 0; cds-WP_002904403.1, NZ_KK737546.1, 483647, 484315, -, 669, 25; cds-WP_004217987.1, NZ_KK737546.1, 484359, 484487, -, 129, 45; cds-WP_013815099.1-11, NZ_KK737546.1, 484550, 485518, -, 969, 7632; cds-WP_032429507.1, NZ_KK737546.1, 485643, 486077, +, 435, 2; cds-WP_002904397.1, NZ_KK737546.1, 486098, 486472, +, 375, 1262; cds-WP_002904394.1, NZ_KK737546.1, 486513, 486731, +, 219, 0; cds-WP_004143566.1, NZ_KK737546.1, 486780, 487679, -, 900, 0; cds-WP_021440091.1, NZ_KK737546.1, 487875, 489071, +, 1197, 0; cds-WP_002904389.1, NZ_KK737546.1, 489201, 489683, +, 483, 0; cds-WP_004176246.1, NZ_KK737546.1, 489719, 490417, -, 699, 5; cds-WP_004148342.1, NZ_KK737546.1, 490427, 491224, -, 798, 0; cds-WP_032429508.1, NZ_KK737546.1, 491224, 492297, -, 1074, 0; cds-WP_015958384.1, NZ_KK737546.1, 492297, 493871, -, 1575, 0; cds-WP_032429509.1, NZ_KK737546.1, 493933, 495204, -, 1272, 0; cds-WP_004148338.1, NZ_KK737546.1, 495376, 496479, -, 1104, 0; cds-
WP_004183961.1, NZ_KK737546.1, 496628, 497026, -, 399, 0; cds-WP_029602386.1, NZ_KK737546.1, 497094, 498191, +, 1098, 37; cds-WP_004205985.1, NZ_KK737546.1, 498160, 498375, +, 216, 2; cds-WP_021440087.1, NZ_KK737546.1, 498428, 498868, -, 441, 95; cds-WP_004183956.1, NZ_KK737546.1, 499122, 500186, -, 1065, 0; cds-WP_021440086.1, NZ_KK737546.1, 500383, 503490, +, 3108, 517; cds-WP_032429510.1, NZ_KK737546.1, 503545, 504810, +, 1266, 995; cds-WP_021440085.1, NZ_KK737546.1, 504841, 505929, -, 1089, 22; cds-WP_004176262.1, NZ_KK737546.1, 506016, 506276, -, 261, 1159; cds-WP_021440083.1, NZ_KK737546.1, 506568, 507434, +, 867, 1034; cds-WP_002210513.1, NZ_KK737546.1, 507455, 508216, -, 762, 1737; cds-AF52_RS0123150, NZ_KK737546.1, 508478, 509380, +, 903, 0; cds-WP_004224682.1, NZ_KK737546.1, 509392, 510657, +, 1266, 0; cds-WP_002210516.1, NZ_KK737546.1, 510650, 511270, +, 621, 0; cds-WP_004176276.1, NZ_KK737546.1, 511275, 512057, +, 783, 4; cds-WP_013815099.1-12, NZ_KK737546.1, 512157, 513125, -, 969, 10989; cds-AF52_RS0123155, NZ_KK737546.1, 513181, 514545, +, 1365, 2; cds-WP_021440081.1, NZ_KK737546.1, 514572, 515498, -, 927, 0; cds-AF52_RS0123160, NZ_KK737546.1, 515809, 516421, +, 613, 0; cds-WP_004195995.1, NZ_KK737546.1, 516452, 516571, -, 120, 0; cds-WP_004143607.1, NZ_KK737546.1, 516972, 518189, +, 1218, 8647; cds-WP_032429511.1, NZ_KK737546.1, 518210, 518722, -, 513, 1; cds-WP_004183942.1, NZ_KK737546.1, 518888, 519361, -, 474, 20; cds-WP_004173721.1, NZ_KK737546.1, 519807, 519941, -, 135, 19; cds-WP_020316505.1, NZ_KK737546.1, 520183, 520305, +, 123, 0; cds-WP_013815099.1-13, NZ_KK737546.1, 520461, 521429, +, 969, 13000; cds-WP_032429512.1, NZ_KK737546.1, 521466, 522074, +, 609, 11; cds-WP_004143611.1, NZ_KK737546.1, 522071, 522193, -, 123, 0; cds-WP_004219446.1, NZ_KK737546.1, 522248, 522376, -, 129, 0; cds-WP_004176282.1, NZ_KK737546.1, 522548, 523222, +, 675, 67; cds-WP_015958377.1, NZ_KK737546.1, 523273, 524115, -, 843, 106; cds-WP_002903735.1, NZ_KK737546.1, 524143, 524529, +, 387, 62; cds-WP_032417100.1, NZ_KK737546.1, 524607, 526652, -, 2046, 327; cds-WP_032429513.1, NZ_KK737546.1, 526783, 527532, +, 750, 123659; cds-WP_002903730.1, NZ_KK737546.1, 527624, 528310, +, 687, 40595; cds-WP_004183938.1, NZ_KK737546.1, 528363, 528794, -, 432, 1; cds-WP_032429514.1, NZ_KK737546.1, 529059, 530522, -, 1464, 80; cds-WP_032429515.1, NZ_KK737546.1, 530776, 532059, -, 1284, 0; cds-WP_072143281.1, NZ_KK737546.1, 532173, 532535, -, 363, 62; cds-WP_002903720.1, NZ_KK737546.1, 532644, 532985, +, 342, 4975; cds-WP_002903719.1, NZ_KK737546.1, 533064, 533624, +, 561, 14243; cds-WP_032428279.1, NZ_KK737546.1, 533618, 534328, -, 711, 44801; cds-WP_002903714.1, NZ_KK737546.1, 534430, 534699, +, 270, 195; cds-WP_032429516.1, NZ_KK737546.1, 534850, 537285, +, 2436, 0; cds-WP_004152235.1, NZ_KK737546.1, 537296, 537913, +, 618, 4; cds-WP_002903710.1, NZ_KK737546.1, 537915, 538772, +, 858, 0; cds-WP_004209765.1, NZ_KK737546.1, 538814, 539422, +, 609, 0; cds-WP_021440073.1, NZ_KK737546.1, 539556, 540851, +, 1296, 2; cds-WP_032428278.1, NZ_KK737546.1, 540804, 541499, -, 696, 784; cds-WP_002903701.1, NZ_KK737546.1, 541627, 542847, -, 1221, 56; cds-WP_002903700.1, NZ_KK737546.1, 542971, 543873, -, 903, 2; cds-WP_002903698.1, NZ_KK737546.1, 544004, 545254, +, 1251, 743; cds-WP_029884324.1, NZ_KK737546.1, 545594, 547081, +, 1488, 5264; cds-WP_002903693.1, NZ_KK737546.1, 547246, 547665, +, 420, 963; cds-WP_013815099.1-14, NZ_KK737546.1, 548708, 549676, -, 969, 7238; cds-WP_050484275.1, NZ_KK737546.1, 549709, 550482, +, 774, 2; cds-WP_004224649.1, NZ_KK737546.1, 550496, 550612, +, 117, 17; cds-WP_032429518.1, NZ_KK737546.1, 550656, 550985, -, 330, 47641; cds-WP_002903681.1, NZ_KK737546.1, 550972, 551334, -, 363, 54050; cds-WP_002903679.1, NZ_KK737546.1, 551777, 552811, +, 1035, 586; cds-WP_004176297.1, NZ_KK737546.1, 553036, 554691, +, 1656, 0; cds-WP_009486289.1, NZ_KK737546.1, 554691, 555533, +, 843, 0; cds-WP_032428276.1, NZ_KK737546.1, 555551, 555850, +, 300, 0; cds-WP_004143105.1, NZ_KK737546.1, 555843, 556676, +, 834, 0; cds-WP_032429519.1, NZ_KK737546.1, 556676, 557476, +, 801, 0; cds-WP_004148291.1, NZ_KK737546.1, 557613, 558572, +, 960, 0; cds-WP_004152239.1, NZ_KK737546.1, 558576, 559193, +, 618, 2; cds-WP_021440066.1, NZ_KK737546.1, 559193, 560095, +, 903, 0; cds-WP_002903632.1, NZ_KK737546.1, 560085, 561011, -, 927, 1390; cds-AF52_RS20250, NZ_KK737546.1, 561169, 562206, -, 1038, 32; cds-AF52_RS20245, NZ_KK737546.1, 562264, 562518, -, 255, 1282; cds-WP_032429520.1, NZ_KK737546.1, 563260, 563512, -, 253, 352; cds-AF52_RS20235, NZ_KK737546.1, 563544, 564125, -, 582, 0; cds-WP_032428274.1, NZ_KK737546.1, 564433, 565356, -, 924, 1879; cds-WP_016529947.1, NZ_KK737546.1, 565520, 565876, -, 357, 0; cds-WP_032429521.1, NZ_KK737546.1, 566032, 567648, +, 1617, 118; cds-WP_004176303.1, NZ_KK737546.1, 567645, 568364, +, 720, 17; cds-WP_020803870.1, NZ_KK737546.1, 568345, 569295, -, 951, 3; cds-WP_004152245.1, NZ_KK737546.1, 569363, 572140, -, 2778, 3; cds-WP_004148278.1, NZ_KK737546.1, 572782, 574293, +, 1512, 0; cds-WP_004152246.1, NZ_KK737546.1, 574348, 576000, +, 1653, 371; cds-WP_032417097.1, NZ_KK737546.1, 576164, 577780, +, 1617, 0; cds-WP_004143067.1, NZ_KK737546.1, 578370, 578759, -, 390, 0; cds-WP_004151556.1, NZ_KK737546.1, 578752, 579516, -, 765, 219; cds-WP_032429522.1, NZ_KK737546.1, 579506, 580858, -, 1353, 35; cds-WP_009308944.1, NZ_KK737546.1, 580868, 582070, -, 1203, 5; cds-WP_032429523.1, NZ_KK737546.1, 582081, 582737, -, 657, 1; cds-WP_002903522.1, NZ_KK737546.1, 582748, 583434, -, 687, 0; cds-WP_004151560.1, NZ_KK737546.1, 583604, 584410, +, 807, 82; cds-WP_032429524.1, NZ_KK737546.1, 584407, 584970, -, 564, 130; cds-WP_002903460.1, NZ_KK737546.1, 585072, 585980, -, 909, 6451; cds-WP_021440059.1, NZ_KK737546.1, 586147, 587457, +, 1311, 5911; cds-WP_020805029.1, NZ_KK737546.1, 587457, 588902, +, 1446, 61502; cds-WP_017879817.1, NZ_KK737546.1, 589022, 590140, +, 1119, 10011; cds-WP_072001564.1, NZ_KK737546.1, 590269, 591363, +, 1095, 31890; cds-WP_072001565.1, NZ_KK737546.1, 591373, 592209, +, 837, 3953; cds-WP_021314545.1, NZ_KK737546.1, 592340, 592711, -, 372, 0; cds-WP_032429525.1, NZ_KK737546.1, 593013, 593564, -, 552, 0; cds-AF52_RS27770, NZ_KK737546.1, 593656, 594012, +, 357, 0; cds-WP_013815099.1-15, NZ_KK737546.1, 596036, 597004, -, 969, 4381; cds-AF52_RS25145, NZ_KK737546.1, 597030, 597290, +, 261, 401; cds-WP_004150800.1, NZ_KK737546.1, 597408, 598658, -, 1251, 472179; cds-WP_004150801.1, NZ_KK737546.1, 598899, 599549, +, 651, 2850; cds-WP_021313538.1, NZ_KK737546.1, 599566, 600024, +, 459, 2; cds-WP_004150803.1, NZ_KK737546.1, 600081, 601187, +, 1107, 14708; cds-WP_004140557.1, NZ_KK737546.1, 601242, 601883, +, 642, 4172; cds-WP_004176557.1, NZ_KK737546.1, 601887, 603257, +, 1371, 44995; cds-WP_004213084.1, NZ_KK737546.1, 603312, 603674, -, 363, 32; cds-WP_004183655.1, NZ_KK737546.1, 603758, 604564, -, 807, 5; cds-WP_004150807.1, NZ_KK737546.1, 604848, 605519, +, 672, 365; cds-WP_004147969.1, NZ_KK737546.1, 605519, 606985, +, 1467, 37800; cds-WP_004176560.1, NZ_KK737546.1, 607071, 608192, +, 1122, 9355; cds-WP_004150810.1, NZ_KK737546.1, 608332, 609564, -, 1233, 0; cds-WP_004150811.1, NZ_KK737546.1, 609823, 610959, +, 1137, 12764; cds-WP_004150812.1, NZ_KK737546.1, 610943, 611800, +, 858, 21; cds-WP_004150813.1, NZ_KK737546.1, 611797, 612582, +, 786, 133; cds-WP_004140590.1, NZ_KK737546.1, 612579, 613625, +, 1047, 7168; cds-WP_032429526.1, NZ_KK737546.1, 613672, 615396, -, 1725, 6; cds-WP_032429527.1, NZ_KK737546.1, 615393, 617021, -, 1629, 9; cds-WP_032429528.1, NZ_KK737546.1, 617259, 619262, -, 2004, 231923; cds-WP_009484250.1, NZ_KK737546.1, 619282, 620235, -, 954, 1; cds-AF52_RS0123220, NZ_KK737546.1, 620263, 620421, -, 159, 4; cds-AF52_RS0123225, NZ_KK737546.1, 620476, 621173, +, 698, 14702; cds-WP_032429529.1, NZ_KK737546.1, 621229, 622248, -, 1020, 0; cds-AF52_RS25155, NZ_KK737546.1, 622341, 622724, -, 384, 10; cds-WP_013815099.1-16, NZ_KK737546.1, 622777, 623745, -, 969, 5558; cds-AF52_RS19995, NZ_KK737546.1, 623776, 625125, -, 1350, 8; cds-WP_004179336.1, NZ_KK737546.1, 625161, 626168, -, 1008, 31393; cds-WP_004211823.1, NZ_KK737546.1, 626411, 627205, +, 795, 16; cds-WP_004140612.1, NZ_KK737546.1, 627241, 628203, +, 963, 562; cds-WP_032429531.1, NZ_KK737546.1, 628228, 628572, -, 345, 3355; cds-WP_004179333.1, NZ_KK737546.1, 628663, 629493, -, 831, 28351; cds-WP_004140619.1, NZ_KK737546.1, 629508, 630419, -, 912, 24908; cds-WP_002900801.1, NZ_KK737546.1, 630468, 631712, -, 1245, 1362; cds-WP_002900798.1, NZ_KK737546.1, 631712, 632413, -, 702, 857; cds-WP_004176566.1, NZ_KK737546.1, 632406, 633605, -, 1200, 2190; cds-WP_004140630.1, NZ_KK737546.1, 633739, 633870, -, 132, 0; cds-WP_016946615.1, NZ_KK737546.1, 633910, 637356, +, 3447, 926; cds-WP_023159039.1, NZ_KK737546.1, 637514, 638479, +, 966, 14631; cds-WP_002900788.1, NZ_KK737546.1, 638608, 638865, -, 258, 4; cds-WP_004147941.1, NZ_KK737546.1, 638963, 639118, +, 156, 49; cds-WP_004140640.1, NZ_KK737546.1, 639115, 639750, +, 636, 41379; cds-WP_004140642.1, NZ_KK737546.1, 639803, 639925, -, 123, 2542; cds-WP_004150817.1, NZ_KK737546.1, 639994, 642183, +, 2190, 370603; cds-WP_002900781.1, NZ_KK737546.1, 642241, 642780, -, 540, 1; cds-WP_002900778.1, NZ_KK737546.1, 642975, 644279, -, 1305, 11; cds-WP_002900775.1, NZ_KK737546.1, 644525, 645067, -, 543, 11783; cds-WP_004147935.1, NZ_KK737546.1, 645103, 646113, -, 1011, 12064; cds-WP_023286229.1, NZ_KK737546.1, 646137, 646964, -, 828, 3747; cds-WP_014342994.1, NZ_KK737546.1, 646945, 647583, -, 639, 3200; cds-WP_002900683.1, NZ_KK737546.1, 647605, 647979, -, 375, 224; cds-WP_002900681.1, NZ_KK737546.1, 647984, 648340, -, 357, 1492; cds-WP_002900680.1, NZ_KK737546.1, 648602, 650035, -, 1434, 128248; cds-WP_009484238.1, NZ_KK737546.1, 650332, 651126, -, 795, 165; cds-WP_021441547.1, NZ_KK737546.1, 651137, 652141, -, 1005, 21061; cds-WP_002900674.1, NZ_KK737546.1, 652138, 652779, -, 642, 26; cds-WP_002900671.1, NZ_KK737546.1, 652769, 653791, -, 1023, 34; cds-WP_002900669.1, NZ_KK737546.1, 653793, 654602, -, 810, 52; cds-WP_032428777.1, NZ_KK737546.1, 654728, 655969, -, 1242, 245600; cds-WP_000103754.1, NZ_KK737546.1, 656059, 656295, -, 237, 173314; cds-WP_002899294.1, NZ_KK737546.1, 656506, 657240, -, 735, 71714; cds-WP_009308402.1, NZ_KK737546.1, 657253, 658182, -, 930, 138442; cds-WP_002899288.1, NZ_KK737546.1, 658198, 659151, -, 954, 62400; cds-WP_004140697.1, NZ_KK737546.1, 659205, 660365, -, 1161, 208019; cds-WP_000290724.1, NZ_KK737546.1, 660506, 660679, -, 174, 392185; cds-WP_002899276.1, NZ_KK737546.1, 660696, 661217, -, 522, 912516; cds-WP_004183641.1, NZ_KK737546.1, 661359, 661943, +, 585, 1; cds-WP_002899271.1, NZ_KK737546.1, 661981, 662934, -, 954, 39; cds-AF52_RS19800, NZ_KK737546.1, 663634, 666601, +, 2968, 267479; cds-WP_014342992.1, NZ_KK737546.1, 666598, 666849, +, 252, 83; cds-WP_004218670.1, NZ_KK737546.1, 666888, 667916, -, 1029, 281; cds-WP_004176581.1, NZ_KK737546.1, 667933, 669147, +, 1215, 2942; cds-WP_002899104.1, NZ_KK737546.1, 669202, 670737, -, 1536, 167525; cds-WP_004140718.1, NZ_KK737546.1, 670849, 671760, -, 912, 5805; cds-WP_004179323.1, NZ_KK737546.1, 671776, 672429, -, 654, 6021; cds-WP_002899099.1, NZ_KK737546.1, 672439, 673023, -, 585, 2071; cds-WP_002899096.1, NZ_KK737546.1, 673253, 674461, +, 1209, 733; cds-WP_004150824.1, NZ_KK737546.1, 674575, 675135, +, 561, 645; cds-WP_032428773.1, NZ_KK737546.1, 675261, 676307, +, 1047, 60431; cds-WP_002898994.1, NZ_KK737546.1, 676380, 676631, +, 252,1169; cds-WP_002898992.1, NZ_KK737546.1, 676934, 677188, +, 255, 7060; cds-WP_014907855.1, NZ_KK737546.1, 677314, 678432, +, 1119, 4660; cds-WP_002898988.1, NZ_KK737546.1, 678504, 678617, +, 114, 4133; cds-WP_032429533.1, NZ_KK737546.1, 678603, 679667, -, 1065, 8100; cds-WP_004140735.1, NZ_KK737546.1, 679873, 680793, +, 921, 77; cds-WP_002898984.1, NZ_KK737546.1, 680965, 682191, +, 1227, 0; cds-WP_002898981.1, NZ_KK737546.1, 682283, 682669, +, 387, 1; cds-WP_002898978.1, NZ_KK737546.1, 682670, 682897, -, 228, 0; cds-WP_002898967.1, NZ_KK737546.1, 682964, 685492, -, 2529, 101042; cds-WP_004183630.1, NZ_KK737546.1, 685485, 687038, -, 1554, 263280; cds-WP_021441537.1, NZ_KK737546.1, 687290, 688450, +, 1161, 5293; cds-WP_004179313.1, NZ_KK737546.1, 688485, 689078, -, 594, 13; cds-WP_021441536.1, NZ_KK737546.1, 689145, 689672, -, 528, 5; cds-WP_004191006.1, NZ_KK737546.1, 689822, 690304, -, 483, 8; cds-WP_004140758.1, NZ_KK737546.1, 690408, 690962, -, 555,
8083; cds-WP_004140764.1, NZ_KK737546.1, 690985, 691722, -, 738, 11609; cds-WP_032429534.1, NZ_KK737546.1, 691811, 692749, -, 939, 798; cds-WP_020804646.1, NZ_KK737546.1, 693463, 694368, -, 906, 0; cds-WP_032428769.1, NZ_KK737546.1, 694483, 695343, +, 861, 2; cds-WP_002898927.1, NZ_KK737546.1, 695362, 696039, +, 678, 42; cds-WP_013815099.1-17, NZ_KK737546.1, 696673, 697641, +, 969, 5470; cds-AF52_RS0123255, NZ_KK737546.1, 697961, 698214, +, 254, 0; cds-WP_148673676.1, NZ_KK737546.1, 698298, 699011, +, 714, 23; cds-WP_072001576.1, NZ_KK737546.1, 699534, 699737, -, 204, 70; cds-WP_020806188.1, NZ_KK737546.1, 699763, 700743, +, 981, 4501; cds-WP_002906471.1, NZ_KK737546.1, 701021, 701311, -, 291, 0; cds-AF52_RS0123260, NZ_KK737546.1, 701331, 702492, -, 1162, 0; cds-WP_187079186.1, NZ_KK737546.1, 702608, 703498, +, 891, 288; cds-WP_032429537.1, NZ_KK737546.1, 703493, 704992, -, 1500, 959; cds-WP_021440209.1, NZ_KK737546.1, 705116, 705691, +, 576, 183; cds-WP_004151231.1, NZ_KK737546.1, 705866, 707248, +, 1383, 0; cds-WP_021440211.1, NZ_KK737546.1, 707363, 711103, +, 3741, 1; cds-WP_004148517.1, NZ_KK737546.1, 711100, 712644, +, 1545, 20; cds-WP_002906547.1, NZ_KK737546.1, 712644, 713339, +, 696, 0; cds-WP_004180107.1, NZ_KK737546.1, 713336, 714016, +, 681, 0; cds-WP_032429538.1, NZ_KK737546.1, 714449, 715294, -, 846, 535; cds-WP_021440213.1, NZ_KK737546.1, 715371, 717026, -, 1656, 0; cds-WP_004189830.1, NZ_KK737546.1, 717084, 718052, -, 969, 0; cds-WP_004189827.1, NZ_KK737546.1, 718049, 718753, -, 705, 49; cds-WP_004151228.1, NZ_KK737546.1, 718965, 719546, +, 582, 933; cds-WP_021440214.1, NZ_KK737546.1, 719670, 719897, -, 228, 1552; cds-WP_021440215.1, NZ_KK737546.1, 719968, 720585, -, 618, 1; cds-WP_014343117.1, NZ_KK737546.1, 720663, 721514, -, 852, 0; cds-WP_002906697.1, NZ_KK737546.1, 721553, 722332, -, 780, 0; cds-WP_021440216.1, NZ_KK737546.1, 722316, 723242, -, 927, 0; cds-WP_021440217.1, NZ_KK737546.1, 723252, 723761, -, 510, 0; cds-WP_032429539.1, NZ_KK737546.1, 723773, 724705, -, 933, 2; cds-WP_032429540.1, NZ_KK737546.1, 724723, 726036, -, 1314, 0; cds-WP_032429541.1, NZ_KK737546.1, 726061, 727182, -, 1122, 0; cds-WP_004898544.1, NZ_KK737546.1, 727172, 728179, -, 1008, 0; cds-WP_004180132.1, NZ_KK737546.1, 728271, 728822, -, 552, 0; cds-WP_021440220.1, NZ_KK737546.1, 729096, 729983, +, 888, 65; cds-WP_002906778.1, NZ_KK737546.1, 730173, 731627, +, 1455, 311; cds-WP_002906779.1, NZ_KK737546.1, 731678, 731899, -, 222, 2945; cds-WP_032429542.1, NZ_KK737546.1, 732068, 733129, -, 1062, 74507; cds-WP_032429543.1, NZ_KK737546.1, 733388, 735493, +, 2106, 28020; cds-AF52_RS0123265, NZ_KK737546.1, 735691, 737396, +, 1706, 1; cds-WP_002906788.1, NZ_KK737546.1, 737494, 737793, -, 300, 9; cds-WP_032428300.1, NZ_KK737546.1, 737883, 738113, -, 231, 0; cds-WP_021440225.1, NZ_KK737546.1, 738075, 739010, +, 936, 122; cds-WP_002906792.1, NZ_KK737546.1, 739074, 740111, -, 1038, 6067; cds-WP_002906794.1, NZ_KK737546.1, 740177, 740758, -, 582, 5343; cds-WP_002906796.1, NZ_KK737546.1, 740921, 741439, +, 519, 350; cds-WP_004180135.1, NZ_KK737546.1, 741436, 741885, +, 450, 5; cds-WP_002906801.1, NZ_KK737546.1, 741894, 742127, -, 234, 34; cds-WP_009484786.1, NZ_KK737546.1, 742144, 743742, -, 1599, 29548; cds-WP_021440226.1, NZ_KK737546.1, 744012, 745445, +, 1434, 3569; cds-WP_004200090.1, NZ_KK737546.1, 745459, 746208, -, 750, 3; cds-AF52_RS25200, NZ_KK737546.1, 746297, 746392, -, 96, 0; cds-WP_013815099.1-18, NZ_KK737546.1, 746451, 747419, -, 969, 2343; cds-AF52_RS0123285, NZ_KK737546.1, 747453, 747536, -, 84, 0; cds-WP_002906891.1, NZ_KK737546.1, 747732, 747824, +, 93, 0; cds-AF52_RS0123290, NZ_KK737546.1, 747881, 748849, +, 969,11731; cds-WP_002906893.1, NZ_KK737546.1, 748942, 749193, -, 252, 106; cds-WP_004189776.1, NZ_KK737546.1, 749331, 749528, -, 198, 0; cds-WP_004175940.1, NZ_KK737546.1, 749677, 749799, +, 123, 0; cds-WP_004175939.1, NZ_KK737546.1, 750140, 751567, -, 1428, 14; cds-WP_004143773.1, NZ_KK737546.1, 751590, 752396, -, 807, 0; cds-WP_032429544.1, NZ_KK737546.1, 752386, 753315, -, 930, 0; cds-WP_004151215.1, NZ_KK737546.1, 753317, 754330, -, 1014, 0; cds-AF52_RS0123305, NZ_KK737546.1, 754347, 755492, -, 1146, 0; cds-WP_032429724.1, NZ_KK737546.1, 755797, 757209, -, 1413, 9231; cds-WP_032429545.1, NZ_KK737546.1, 757897, 759294, +, 1398, 893; cds-WP_020802114.1, NZ_KK737546.1, 759294, 760304, +, 1011, 0; cds-WP_004180143.1, NZ_KK737546.1, 760304, 760429, +, 126, 0; cds-WP_187079190.1, NZ_KK737546.1, 760525, 760692, +, 168, 110; cds-WP_013815099.1-19, NZ_KK737546.1, 760738, 761706, -, 969, 7026; cds-WP_077254734.1, NZ_KK737546.1, 761708, 761899, -, 192, 0; cds-WP_004143781.1, NZ_KK737546.1, 762045, 762275, +, 231, 3064; cds-WP_032429546.1, NZ_KK737546.1, 762288, 764249, -, 1962, 3765; cds-WP_020806151.1, NZ_KK737546.1, 764283, 764450, +, 168, 3041; cds-WP_002907473.1, NZ_KK737546.1, 764461, 765030, -, 570, 14207; cds-WP_032428296.1, NZ_KK737546.1, 765216, 766382, +, 1167, 2; cds-WP_004151213.1, NZ_KK737546.1, 766359, 767228, -, 870, 906; cds-WP_004151212.1, NZ_KK737546.1, 767409, 768305, +, 897, 0; cds-WP_002907480.1, NZ_KK737546.1, 768352, 769020, -, 669, 318; cds-WP_021440235.1, NZ_KK737546.1, 769281, 769877, -, 597, 23; cds-WP_002907483.1, NZ_KK737546.1, 769874, 770878, -, 1005, 0; cds-WP_004891515.1, NZ_KK737546.1, 770990, 771970, +, 981, 319; cds-WP_002907487.1, NZ_KK737546.1, 771956, 772534, -, 579, 173477; cds-WP_002907489.1, NZ_KK737546.1, 772588, 774243, -, 1656, 68573; cds-WP_032428297.1, NZ_KK737546.1, 774681, 775769, +, 1089, 1; cds-WP_021440237.1, NZ_KK737546.1, 775816, 777321, -, 1506, 14; cds-WP_002907549.1, NZ_KK737546.1, 777363, 778706, -, 1344, 1301; cds-WP_021440238.1, NZ_KK737546.1, 778946, 779686, +, 741, 6; cds-WP_004143799.1, NZ_KK737546.1, 779683, 780303, +, 621, 1; cds-WP_004143800.1, NZ_KK737546.1, 780481, 781404, +, 924, 144820; cds-WP_021440239.1, NZ_KK737546.1, 781704, 784076, +, 2373, 48464; cds-WP_004143804.1, NZ_KK737546.1, 784146, 785264, -, 1119, 61539; cds-WP_002907563.1, NZ_KK737546.1, 785289, 785564, -, 276, 0; cds-WP_002907640.1, NZ_KK737546.1, 785669, 786196, -, 528, 405; cds-WP_004143806.1, NZ_KK737546.1, 786456, 786818, +, 363, 71; cds-WP_002907644.1, NZ_KK737546.1, 786828, 787013, -, 186, 586; cds-WP_032429547.1, NZ_KK737546.1, 787257, 787721, -, 465, 3680; cds-WP_032428298.1, NZ_KK737546.1, 787915, 789069, +, 1155, 82694; cds-WP_004151209.1, NZ_KK737546.1, 789227, 790228, +, 1002, 49322; cds-WP_021440243.1, NZ_KK737546.1, 790264, 791304, -, 1041, 1172; cds-WP_004227851.1, NZ_KK737546.1, 791495, 791632, +, 138, 175; cds-WP_021440244.1, NZ_KK737546.1, 791748, 793118, +, 1371, 1057; cds-WP_002907652.1, NZ_KK737546.1, 793335, 793550, +, 216, 2160; cds-WP_002907654.1, NZ_KK737546.1, 793626, 794066, +, 441, 10604; cds-WP_002907658.1, NZ_KK737546.1, 794145, 794726, +, 582, 7960; cds-WP_004143816.1, NZ_KK737546.1, 794726, 795304, +, 579, 13489; cds-AF52_RS19200, NZ_KK737546.1, 795297, 797532, +, 2236, 687; cds-WP_002907730.1, NZ_KK737546.1, 797533, 798585, +, 1053, 4572; cds-WP_002907731.1, NZ_KK737546.1, 798596, 799216, +, 621, 996; cds-WP_002907733.1, NZ_KK737546.1, 799219, 799917, +, 699, 13708; cds-WP_032429549.1, NZ_KK737546.1, 799919, 800554, +, 636, 9239; cds-WP_004151206.1, NZ_KK737546.1, 801162, 802667, +, 1506, 157144; cds-WP_021440245.1, NZ_KK737546.1, 802778, 803383, +, 606, 27555; cds-WP_032428303.1, NZ_KK737546.1, 803426, 804286, -, 861, 4333; cds-WP_032429550.1, NZ_KK737546.1, 804348, 805622, -, 1275, 27416; cds-WP_032428304.1, NZ_KK737546.1, 805747, 806403, -, 657, 3050; cds-WP_032428306.1, NZ_KK737546.1, 806461, 806784, -, 324, 292; cds-WP_004143831.1, NZ_KK737546.1, 806877, 808001, -, 1125, 73; cds-WP_002907749.1, NZ_KK737546.1, 808269, 808736, +, 468, 2665; cds-AF52_RS19130, NZ_KK737546.1, 808959, 810134, +, 1176, 95; cds-WP_013815099.1-20, NZ_KK737546.1, 810186, 811154, -, 969, 3472; cds-AF52_RS25210, NZ_KK737546.1, 811180, 811386, +, 207, 17; cds-WP_004143833.1, NZ_KK737546.1, 811420, 811860, -, 441, 21496; cds-WP_002907752.1, NZ_KK737546.1, 812033, 812269, +, 237, 132; cds-WP_004143835.1, NZ_KK737546.1, 812280, 813176, +, 897, 11038; cds-WP_002907754.1, NZ_KK737546.1, 813176, 815200, +, 2025, 9935; cds-WP_032429551.1, NZ_KK737546.1, 815193, 815711, -, 519, 13894; cds-WP_021440254.1, NZ_KK737546.1, 815781, 816677, -, 897, 35155; cds-WP_004189722.1, NZ_KK737546.1, 816726, 816968, -, 243, 0; cds-WP_004175857.1, NZ_KK737546.1, 817108, 817707, +, 600, 15; cds-WP_002907763.1, NZ_KK737546.1, 817761, 818858, +, 1098, 1438; cds-WP_013815099.1-21, NZ_KK737546.1, 818914, 819882, +, 969, 4572; cds-WP_002907764.1, NZ_KK737546.1, 820003, 820410, +, 408, 3; cds-WP_002907766.1, NZ_KK737546.1, 820548, 821195, +, 648, 108; cds-WP_032429552.1, NZ_KK737546.1, 821243, 821590, -, 348, 620; cds-WP_004151204.1, NZ_KK737546.1, 821931, 822803, +, 873, 14654; cds-WP_004200127.1, NZ_KK737546.1, 822758, 823243, -, 486, 354; cds-WP_002907771.1, NZ_KK737546.1, 823713, 824294, +, 582, 53581; cds-WP_002907773.1, NZ_KK737546.1, 824356, 825522, -, 1167, 3314; cds-WP_004152976.1, NZ_KK737546.1, 825690, 825779, -, 90, 210; cds-WP_002907778.1, NZ_KK737546.1, 826075, 827100, +, 1026, 74; cds-WP_004180170.1, NZ_KK737546.1, 827027, 828031, -, 1005, 4; cds-WP_002907785.1, NZ_KK737546.1, 828144, 829325, +, 1182, 2384; cds-WP_032429553.1, NZ_KK737546.1, 829618, 830766, +, 1149, 16424; cds-WP_021440257.1, NZ_KK737546.1, 830803, 831438, -, 636, 2685; cds-WP_032429554.1, NZ_KK737546.1, 831668, 833041, +, 1374, 4164; cds-WP_004180172.1, NZ_KK737546.1, 833217, 833453, +, 237, 0; cds-AF52_RS0123325, NZ_KK737546.1, 833594, 834171, -, 578, 247; cds-AF52_RS25220, NZ_KK737546.1, 835237, 835697, -, 461, 0; cds-WP_032428308.1, NZ_KK737546.1, 836525, 837277, -, 753, 17; cds-WP_004180175.1, NZ_KK737546.1, 837376, 838302, +, 927, 0; cds-WP_002907799.1, NZ_KK737546.1, 838299, 839183, -, 885, 16; cds-WP_021440259.1, NZ_KK737546.1, 839340, 839855, +, 516, 0; cds-WP_004184289.1, NZ_KK737546.1, 839863, 840699, +, 837, 10423; cds-WP_002907802.1, NZ_KK737546.1, 840787, 840936, -, 150, 627; cds-WP_004143863.1, NZ_KK737546.1, 841338, 841772, +, 435, 0; cds-WP_021440261.1, NZ_KK737546.1, 841818, 842534, +, 717, 3; cds-WP_002907807.1, NZ_KK737546.1, 842619, 842828, +, 210, 0; cds-WP_002907811.1, NZ_KK737546.1, 843141, 843776, -, 636, 10736; cds-WP_002907812.1, NZ_KK737546.1, 844026, 844655, +, 630, 11671; cds-WP_004189677.1, NZ_KK737546.1, 844660, 845430, -, 771, 0; cds-WP_004175834.1, NZ_KK737546.1, 845522, 845905, +, 384, 1; cds-WP_004175829.1, NZ_KK737546.1, 845937, 846620, +, 684, 9; cds-WP_032428309.1, NZ_KK737546.1, 846965, 848668, +, 1704, 5; cds-WP_004175825.1, NZ_KK737546.1, 848712, 849671, +, 960, 0; cds-WP_016197619.1, NZ_KK737546.1, 849668, 850618, +, 951, 0; cds-WP_021440263.1, NZ_KK737546.1, 850615, 851595, +, 981, 0; cds-WP_021440264.1, NZ_KK737546.1, 851592, 852563, +, 972, 0; cds-WP_002907918.1, NZ_KK737546.1, 852929, 853456, +, 528, 8187; cds-WP_032429555.1, NZ_KK737546.1, 853574, 854062, +, 489, 73231; cds-AF52_RS25225, NZ_KK737546.1, 854099, 854194, -, 96, 4; cds-AF52_RS0123345, NZ_KK737546.1, 854208, 855739, -, 1532, 3; cds-WP_021440267.1, NZ_KK737546.1, 855856, 856773, +, 918, 5; cds-WP_021440268.1, NZ_KK737546.1, 856847, 857836, -, 990, 0; cds-WP_032429556.1, NZ_KK737546.1, 857857, 859275, -, 1419, 5; cds-WP_032428310.1, NZ_KK737546.1, 859287, 860276, -, 990, 116; cds-WP_021440271.1, NZ_KK737546.1, 860571, 861470, -, 900, 609; cds-WP_032428312.1, NZ_KK737546.1, 861678, 862646, +, 969, 0; cds-WP_021440273.1, NZ_KK737546.1, 862658, 863941, +, 1284, 0; cds-WP_002908128.1, NZ_KK737546.1, 863931, 864410, +, 480, 0; cds-WP_021440274.1, NZ_KK737546.1, 864407, 865288, +, 882, 0; cds-WP_002908130.1, NZ_KK737546.1, 865288, 866271, +, 984, 2; cds-WP_032429558.1, NZ_KK737546.1, 866268, 867098, +, 831, 0; cds-WP_002908134.1, NZ_KK737546.1, 867100, 867915, +, 816, 0; cds-WP_032429559.1, NZ_KK737546.1, 867942, 869363, +, 1422, 1; cds-WP_026005880.1, NZ_KK737546.1, 869443, 869691, +, 249, 0; cds-WP_004145368.1, NZ_KK737546.1, 869857, 870765, -, 909, 78716; cds-WP_002908189.1, NZ_KK737546.1, 871055, 871195, +, 141, 170; cds-WP_004189584.1, NZ_KK737546.1, 871242, 872132, -, 891, 0; cds-WP_004175762.1, NZ_KK737546.1, 872273, 872980, +, 708, 0; cds-WP_021440277.1, NZ_KK737546.1, 872982, 873638, +, 657, 0; cds-WP_032429560.1, NZ_KK737546.1, 873648, 874829, +, 1182, 7; cds-WP_004891326.1, NZ_KK737546.1, 874841, 875764, +, 924, 5; cds-WP_032428313.1, NZ_KK737546.1, 875812, 877203, +, 1392, 0; cds-WP_002908202.1,
NZ_KK737546.1, 877227, 877997, +, 771, 0; cds-WP_004145376.1, NZ_KK737546.1, 878017, 878889, -, 873, 0; cds-WP_002908205.1, NZ_KK737546.1, 878996, 879775, +, 780, 0; cds-WP_004200174.1, NZ_KK737546.1, 879785, 881464, +, 1680, 0; cds-WP_004151179.1, NZ_KK737546.1, 881488, 882258, +, 771, 904; cds-WP_013815099.1-22, NZ_KK737546.1, 883311, 884279, -, 969, 6246; cds-AF52_RS0123360, NZ_KK737546.1, 884312, 884632, +, 321, 618; cds-WP_032428316.1, NZ_KK737546.1, 885134, 885976, -, 843, 0; cds-WP_014599186.1, NZ_KK737546.1, 885973, 886419, -, 447, 0; cds-WP_002908216.1, NZ_KK737546.1, 886431, 886985, -, 555, 144; cds-WP_021440283.1, NZ_KK737546.1, 886982, 888934, -, 1953, 4307; cds-WP_021440284.1, NZ_KK737546.1, 888931, 889389, -, 459, 0; cds-WP_021440285.1, NZ_KK737546.1, 889373, 889603, -, 231, 0; cds-WP_004189553.1, NZ_KK737546.1, 889600, 890337, -, 738, 291; cds-WP_032428317.1, NZ_KK737546.1, 890387, 891046, -, 660, 0; cds-WP_004151165.1, NZ_KK737546.1, 891043, 891666, -, 624, 0; cds-WP_182920176.1, NZ_KK737546.1, 891750, 893090, -, 1341, 45; cds-WP_002908285.1, NZ_KK737546.1, 893101, 894885, -, 1785, 0; cds-WP_004217200.1, NZ_KK737546.1, 894888, 895643, -, 756, 1; cds-WP_002908289.1, NZ_KK737546.1, 896145, 896516, +, 372, 98; cds-WP_002908292.1, NZ_KK737546.1, 896524, 897594, -, 1071, 49; cds-WP_002908294.1, NZ_KK737546.1, 897587, 899356, -, 1770, 8800; cds-WP_002908297.1, NZ_KK737546.1, 899430, 900518, -, 1089, 108729; cds-WP_021313976.1, NZ_KK737546.1, 900821, 901900, +, 1080, 4684; cds-WP_021440289.1, NZ_KK737546.1, 902147, 904297, +, 2151, 17; cds-WP_014343153.1, NZ_KK737546.1, 904340, 904501, -, 162, 0; cds-AF52_RS18635, NZ_KK737546.1, 904465, 905497, -, 1033, 55; cds-WP_004148650.1, NZ_KK737546.1, 905793, 906992, +, 1200, 2; cds-WP_004175720.1, NZ_KK737546.1, 907108, 907542, +, 435, 58; cds-WP_032428318.1, NZ_KK737546.1, 908108, 909340, +, 1233, 0; cds-WP_002908370.1, NZ_KK737546.1, 909377, 909769, +, 393, 0; cds-WP_021440293.1, NZ_KK737546.1, 909898, 910485, -, 588, 0; cds-WP_002908374.1, NZ_KK737546.1, 910785, 911507, -, 723, 35; cds-WP_032429562.1, NZ_KK737546.1, 911494, 912258, -, 765, 16; cds-WP_032428319.1, NZ_KK737546.1, 912322, 913089, -, 768, 1568; cds-WP_002908378.1, NZ_KK737546.1, 913182, 914084, -, 903, 0; cds-WP_004148675.1, NZ_KK737546.1, 914081, 915058, -, 978, 3; cds-WP_021440296.1, NZ_KK737546.1, 915048, 916100, -, 1053, 17; cds-WP_004213478.1, NZ_KK737546.1, 916097, 917122, -, 1026, 0; cds-WP_002908416.1, NZ_KK737546.1, 917119, 917940, -, 822, 3; cds-WP_004891261.1, NZ_KK737546.1, 918446, 920518, +, 2073, 0; cds-WP_004175711.1, NZ_KK737546.1, 920627, 921400, -, 774, 0; cds-WP_004148681.1, NZ_KK737546.1, 921394, 922389, -, 996, 0; cds-WP_032429563.1, NZ_KK737546.1, 922370, 923407, -, 1038, 0; cds-WP_002908454.1, NZ_KK737546.1, 923404, 924366, -, 963, 0; cds-WP_032429564.1, NZ_KK737546.1, 924372, 925523, -, 1152, 0; cds-WP_016947511.1, NZ_KK737546.1, 925520, 926605, -, 1086, 3; cds-WP_032429565.1, NZ_KK737546.1, 926750, 927643, -, 894, 0; cds-WP_002908526.1, NZ_KK737546.1, 927750, 928352, +, 603, 0; cds-WP_021440299.1, NZ_KK737546.1, 928358, 929938, +, 1581, 1; cds-WP_021440300.1, NZ_KK737546.1, 929951, 930676, -, 726, 34; cds-AF52_RS0123375, NZ_KK737546.1, 930779, 931975, -, 1197, 0; cds-WP_004180340.1, NZ_KK737546.1, 932051, 933067, -, 1017, 14; cds-WP_021440302.1, NZ_KK737546.1, 933064, 934014, -, 951, 0; cds-WP_032429566.1, NZ_KK737546.1, 934007, 934813, -, 807, 1; cds-WP_004175683.1, NZ_KK737546.1, 934824, 935690, -, 867, 0; cds-WP_021440303.1, NZ_KK737546.1, 935708, 936652, -, 945, 0; cds-WP_024622675.1, NZ_KK737546.1, 936654, 938318, -, 1665, 0; cds-WP_004184404.1, NZ_KK737546.1, 938496, 939308, +, 813, 1; cds-WP_016530930.1, NZ_KK737546.1, 939400, 940458, +, 1059, 287; cds-WP_004151840.1, NZ_KK737546.1, 940448, 941455, +, 1008, 8; cds-WP_004151841.1, NZ_KK737546.1, 941452, 942201, +, 750, 0; cds-WP_014907413.1, NZ_KK737546.1, 942185, 942928, -, 744, 8; cds-WP_004175664.1, NZ_KK737546.1, 942985, 943821, -, 837, 393; cds-WP_004148701.1, NZ_KK737546.1, 943837, 945159, -, 1323, 2; cds-WP_032429567.1, NZ_KK737546.1, 945231, 947084, -, 1854, 3; cds-WP_002908844.1, NZ_KK737546.1, 947211, 947849, +, 639, 17574; cds-WP_004180357.1, NZ_KK737546.1, 947867, 948079, -, 213, 4001; cds-WP_014907411.1, NZ_KK737546.1, 948069, 948227, -, 159, 18; cds-WP_004217210.1, NZ_KK737546.1, 948316, 948447, -, 132, 1; cds-WP_002908859.1, NZ_KK737546.1, 948585, 949997, +, 1413, 102382; cds-WP_032429569.1, NZ_KK737546.1, 950310, 950546, +, 237, 179271; cds-WP_004143241.1, NZ_KK737546.1, 950608, 951594, -, 987, 6420; cds-WP_002908865.1, NZ_KK737546.1, 951694, 952110, -, 417, 0; cds-WP_002908867.1, NZ_KK737546.1, 952122, 953342, -, 1221, 0; cds-AF52_RS18405, NZ_KK737546.1, 953339, 954046, -, 708, 0; cds-WP_013815099.1-23, NZ_KK737546.1, 954079, 955047, +, 969, 5830; cds-AF52_RS18395, NZ_KK737546.1, 955104, 955679, -, 576, 92; cds-WP_002908876.1, NZ_KK737546.1, 955654, 956400, -, 747, 2; cds-WP_002908877.1, NZ_KK737546.1, 956417, 957904, -, 1488, 4136; cds-WP_004143232.1, NZ_KK737546.1, 957907, 958284, -, 378, 914; cds-WP_002908880.1, NZ_KK737546.1, 958767, 958964, -, 198, 922; cds-WP_014343173.1, NZ_KK737546.1, 958963, 959100, +, 138, 346; cds-WP_004143228.1, NZ_KK737546.1, 959201, 959596, +, 396, 0; cds-WP_002908882.1, NZ_KK737546.1, 959593, 960225, +, 633, 2; cds-WP_004151846.1, NZ_KK737546.1, 960480, 961664, +, 1185, 195; cds-WP_021440309.1, NZ_KK737546.1, 961720, 964812, -, 3093, 0; cds-WP_032429570.1, NZ_KK737546.1, 964809, 965918, -, 1110, 0; cds-WP_020805192.1, NZ_KK737546.1, 965993, 966529, -, 537, 3; cds-WP_021440311.1, NZ_KK737546.1, 966703, 967698, -, 996, 1; cds-WP_004175653.1, NZ_KK737546.1, 967795, 968706, +, 912, 3; cds-WP_004180366.1, NZ_KK737546.1, 968865, 969863, +, 999, 27; cds-WP_002908998.1, NZ_KK737546.1, 969863, 970900, +, 1038, 9390; cds-WP_004212506.1, NZ_KK737546.1, 970900, 971661, +, 762, 19798; cds-WP_182920187.1, NZ_KK737546.1, 971718, 972905, +, 1188, 283; cds-WP_002909005.1, NZ_KK737546.1, 972988, 974019, -, 1032, 21; cds-WP_002909008.1, NZ_KK737546.1, 974043, 975023, -, 981, 1958; cds-WP_004212509.1, NZ_KK737546.1, 975593, 975913, +, 321, 305; cds-WP_002909014.1, NZ_KK737546.1, 976011, 976259, -, 249, 818; cds-WP_002909015.1, NZ_KK737546.1, 976464, 976874, -, 411, 161; cds-WP_004175614.1, NZ_KK737546.1, 976871, 979927, -, 3057, 9433; cds-WP_004148729.1, NZ_KK737546.1, 980146, 981258, +, 1113, 6612; cds-WP_002909055.1, NZ_KK737546.1, 981641, 984019, -, 2379, 329014; cds-WP_009484225.1, NZ_KK737546.1, 984095, 984214, +, 120, 1; cds-WP_002909061.1, NZ_KK737546.1, 984359, 985192, +, 834, 5697; cds-WP_021440317.1, NZ_KK737546.1, 985347, 986393, +, 1047, 10058; cds-WP_002909070.1, NZ_KK737546.1, 986501, 986728, +, 228, 862; cds-WP_021440318.1, NZ_KK737546.1, 986758, 988200, -, 1443, 15428; cds-WP_002909082.1, NZ_KK737546.1, 988314, 988778, -, 465, 1604; cds-WP_020803964.1, NZ_KK737546.1, 988860, 989609, -, 750, 139; cds-WP_004180371.1, NZ_KK737546.1, 989609, 990160, -, 552, 2; cds-WP_002909085.1, NZ_KK737546.1, 990397, 991197, +, 801, 15879; cds-WP_004151850.1, NZ_KK737546.1, 991234, 992220, -, 987, 1; cds-WP_014907393.1, NZ_KK737546.1, 992342, 993775, -, 1434, 1; cds-WP_002909089.1, NZ_KK737546.1, 994056, 994970, +, 915, 85800; cds-WP_020802655.1, NZ_KK737546.1, 995020, 996273, +, 1254, 427; cds-WP_021440321.1, NZ_KK737546.1, 996270, 996623, +, 354, 541; cds-WP_002909098.1, NZ_KK737546.1, 996676, 996975, -, 300, 631; cds-WP_015958605.1, NZ_KK737546.1, 996980, 999367, -, 2388, 80582; cds-WP_002909105.1, NZ_KK737546.1, 999383, 1000366, -, 984, 26282; cds-WP_001386830.1, NZ_KK737546.1, 1000505, 1000549, -, 45, 0; cds-WP_000124850.1, NZ_KK737546.1, 1000673, 1001029, -, 357, 652392; cds-WP_001124225.1, NZ_KK737546.1, 1001080, 1001277, -, 198, 475630; cds-WP_004189469.1, NZ_KK737546.1, 1001370, 1001912, -, 543, 464925; cds-WP_032429575.1, NZ_KK737546.1, 1001916, 1003844, -, 1929, 745117; cds-WP_002910028.1, NZ_KK737546.1, 1004276, 1004509, -, 234, 45; cds-WP_002910030.1, NZ_KK737546.1, 1004781, 1005539, -, 759, 78; cds-WP_032429576.1, NZ_KK737546.1, 1005896, 1006828, +, 933, 3; cds-WP_004184572.1, NZ_KK737546.1, 1006923, 1007207, +, 285, 5; cds-WP_032429577.1, NZ_KK737546.1, 1007313, 1008185, +, 873, 5703; cds-WP_014343181.1, NZ_KK737546.1, 1008229, 1009017, -, 789, 141; cds-WP_002910042.1, NZ_KK737546.1, 1009186, 1009482, +, 297, 1552; cds-WP_032103891.1, NZ_KK737546.1, 1009595, 1009735, +, 141, 5; cds-WP_072271743.1, NZ_KK737546.1, 1010163, 1010810, +, 648, 0; cds-WP_004194006.1, NZ_KK737546.1, 1011313, 1011486, -, 174, 3; cds-WP_002910077.1, NZ_KK737546.1, 1011630, 1013090, +, 1461, 464; cds-WP_002910079.1, NZ_KK737546.1, 1013125, 1013454, +, 330, 724; cds-WP_002910080.1, NZ_KK737546.1, 1013517, 1014344, -, 828, 15; cds-WP_002910083.1, NZ_KK737546.1, 1014400, 1015404, -, 1005, 2134; cds-WP_004151853.1, NZ_KK737546.1, 1015401, 1016414, -, 1014,501; cds-WP_004145396.1, NZ_KK737546.1, 1016426, 1017334, -, 909, 6871; cds-WP_002910089.1, NZ_KK737546.1, 1017349, 1018269, -, 921, 8395; cds-WP_004148754.1, NZ_KK737546.1, 1018355, 1019989, -, 1635, 238670; cds-WP_002910095.1, NZ_KK737546.1, 1020727, 1021374, -, 648, 0; cds-WP_004145398.1, NZ_KK737546.1, 1021558, 1021773, -, 216, 0; cds-WP_002910097.1, NZ_KK737546.1, 1021851, 1024526, +, 2676, 228481; cds-WP_002910100.1, NZ_KK737546.1, 1024674, 1025291, -, 618, 212; cds-WP_004145401.1, NZ_KK737546.1, 1025299, 1025430, +, 132, 0; cds-WP_002910103.1, NZ_KK737546.1, 1025853, 1026260, +, 408, 19552; cds-WP_002910105.1, NZ_KK737546.1, 1026382, 1027284, -, 903, 333596; cds-WP_002910107.1, NZ_KK737546.1, 1027482, 1028495, -, 1014, 3568; cds-WP_002910108.1, NZ_KK737546.1, 1028585, 1029487, -, 903, 41826; cds-WP_032429578.1, NZ_KK737546.1, 1029600, 1030058, +, 459, 14422; cds-WP_004151854.1, NZ_KK737546.1, 1030101, 1030943, +, 843, 16212; cds-WP_032428322.1, NZ_KK737546.1, 1031951, 1032394, -, 444, 4; cds-WP_021440328.1, NZ_KK737546.1, 1032502, 1033398, +, 897, 317; cds-WP_032429579.1, NZ_KK737546.1, 1033395, 1034072, -, 678, 4; cds-WP_002910191.1, NZ_KK737546.1, 1034072, 1034782, -, 711, 0; cds-WP_019705142.1, NZ_KK737546.1, 1034779, 1036314, -, 1536, 176; cds-WP_004180382.1, NZ_KK737546.1, 1036311, 1040054, -, 3744, 3006; cds-WP_032428329.1, NZ_KK737546.1, 1040221, 1040334, -, 114, 0; cds-WP_032428323.1, NZ_KK737546.1, 1040460, 1041848, -, 1389, 7608; cds-WP_004175566.1, NZ_KK737546.1, 1042160, 1043938, +, 1779, 18; cds-WP_002910198.1, NZ_KK737546.1, 1043949, 1044599, +, 651, 2312; cds-WP_002910201.1, NZ_KK737546.1, 1044596, 1045978, -, 1383, 605; cds-WP_004175564.1, NZ_KK737546.1, 1046130, 1048730, -, 2601, 0; cds-WP_021440334.1, NZ_KK737546.1, 1048730, 1052797, -, 4068, 0; cds-WP_004145418.1, NZ_KK737546.1, 1052808, 1053596, -, 789, 0; cds-WP_004152255.1, NZ_KK737546.1, 1053606, 1054490, -, 885, 0; cds-WP_032429580.1, NZ_KK737546.1, 1054492, 1055748, -, 1257, 0; cds-WP_021440335.1, NZ_KK737546.1, 1055991, 1057172, -, 1182, 0; cds-WP_002910380.1, NZ_KK737546.1, 1057240, 1057593, +, 354, 63; cds-WP_002910387.1, NZ_KK737546.1, 1057831,1058073, +, 243, 18906; cds-WP_004152258.1, NZ_KK737546.1, 1058291, 1058971, -, 681, 10; cds-WP_002910389.1, NZ_KK737546.1, 1059108, 1059338, -, 231, 6; cds-WP_002910392.1, NZ_KK737546.1, 1059602, 1060702, +, 1101, 18136; cds-WP_002910393.1, NZ_KK737546.1, 1060789, 1061643, -, 855, 69718; cds-WP_032429581.1, NZ_KK737546.1, 1061683, 1062495, -, 813, 12634; cds-WP_004145428.1, NZ_KK737546.1, 1062499, 1062891, -, 393, 528; cds-WP_004898979.1, NZ_KK737546.1, 1062891, 1063739, -, 849, 13; cds-WP_002910403.1, NZ_KK737546.1, 1063739, 1064821, -, 1083, 29470; cds-WP_002910404.1, NZ_KK737546.1, 1064864, 1066120, -, 1257, 12209; cds-WP_002910405.1, NZ_KK737546.1, 1066391, 1067002, +, 612, 2632; cds-WP_004898981.1, NZ_KK737546.1, 1067002, 1067850, +, 849, 67817; cds-WP_002910407.1, NZ_KK737546.1, 1068034, 1068981, +, 948, 311505; cds-WP_004200291.1, NZ_KK737546.1, 1069106, 1070785, +, 1680, 1016; cds-WP_004175547.1, NZ_KK737546.1, 1070786, 1071832, -, 1047, 2; cds-WP_002910437.1, NZ_KK737546.1, 1072054, 1072329, -, 276, 11436; cds-WP_004180389.1, NZ_KK737546.1, 1072602, 1073186, +, 585, 29868; cds-WP_002910443.1, NZ_KK737546.1, 1073304, 1074395, +, 1092, 146825; cds-WP_004145442.1, NZ_KK737546.1, 1074477, 1074806, -, 330, 19; cds-WP_002910453.1, NZ_KK737546.1, 1074890, 1075804, -, 915, 9778; cds-WP_032429582.1, NZ_KK737546.1, 1075936, 1077351, -, 1416, 8; cds-WP_004175538.1, NZ_KK737546.1, 1077371,
1077814, -, 444, 6217; cds-WP_004175532.1, NZ_KK737546.1, 1077817, 1078353, -, 537, 0; cds-WP _021440342.1, NZ_KK737546.1, 1078334, 1079350, -, 1017, 0; cds-AF52_RS17835, NZ_KK737546.1, 1079380, 1081143, -, 1764, 0; cds-WP_171991785.1, NZ_KK737546.1, 1081277, 1084693, -, 3417, 69; cds-WP_004899005.1, NZ_KK737546.1, 1084708, 1085919, -, 1212, 3; cds-WP _004899008.1, NZ_KK737546.1, 1085924, 1086181, -, 258, 0; cds-WP _013815099.1-24, NZ_KK737546.1, 1086223, 1087191, +, 969, 4297; cds-WP_071603213.1, NZ_KK737546.1, 1087273, 1088052, -, 780, 0; cds-AF52_RS25250, NZ_KK737546.1, 1088184, 1089413, -, 1230, 0; cds-AF52_RS26885, NZ_KK737546.1, 1089482, 1089616, +, 135, 61; cds-WP_187079183.1, NZ_KK737546.1, 1089947, 1090528, -, 582, 13; cds-AF52_RS0123460, NZ_KK737546.1, 1090628, 1090912, -, 285, 4597; cds-AF52_RS26890, NZ_KK737546.1, 1095758, 1095891, -, 134, 838; cds-AF52_RS25260, NZ_KK737546.1, 1096147, 1096341, +, 195, 2; cds-WP _050484277.1, NZ_KK737546.1, 1096700, 1097497, -, 798, 25; cds-AF52_RS26910, NZ_KK737546.1, 1101418, 1101547, -, 130, 63; cds-WP _024622687.1, NZ_KK737546.1, 1101978, 1103318, +, 1341, 0; cds-WP_032429587.1, NZ_KK737546.1, 1103315, 1103968, +, 654, 0; cds-WP_032429588.1, NZ_KK737546.1, 1103972, 1105669, +, 1698, 0; cds-AF52_RS27855, NZ_KK737546.1, 1105856, 1105980, +, 125, 0; cds-AF52_RS17780, NZ_KK737546.1, 1106131, 1108194, +, 2064, 10; cds-WP _013815099.1-25, NZ_KK737546.1, 1108246, 1109214, -, 969, 3267; cds-WP_050484282.1, NZ_KK737546.1, 1109194, 1110111, +, 918, 5511; cds-WP _032429589.1, NZ_KK737546.1, 1110157, 1110477, -, 321, 59; cds-WP _032429590.1, NZ_KK737546.1, 1110545, 1110808, -, 264, 0; cds-WP_032429591.1, NZ_KK737546.1, 1110819, 1111511, -, 693, 0; cds-WP_032429592.1, NZ_KK737546.1, 1111564, 1113084, -, 1521, 77; cds-WP _032429593.1, NZ_KK737546.1, 1113085, 1114761, -, 1677, 0; cds-WP _032429594.1, NZ_KK737546.1, 1114766, 1115335, -, 570, 0; cds-WP _032429596.1, NZ_KK737546.1, 1115340, 1115555, -, 216, 0; cds-WP_087749278.1, NZ_KK737546.1, 1115890, 1116027, -, 138, 0; cds-WP_032429598.1, NZ_KK737546.1, 1116030, 1116497, -, 468, 3; cds-WP_020804048.1, NZ_KK737546.1, 1116494, 1117024, -, 531, 4; cds-WP _004151282.1, NZ_KK737546.1, 1117027, 1117275, -, 249, 0; cds-WP _032429600.1, NZ _KK737546.1, 1118641, 1118994, -, 354, 7973; cds-WP _004884209.1, NZ_KK737546.1, 1119587, 1120087, -, 501, 0; cds-WP _032429601.1, NZ _KK737546.1, 1120084, 1120224, -, 141, 0; cds-WP _032429603.1, NZ_KK737546.1, 1120221, 1120583, -, 363, 0; cds-WP_004141687.1, NZ_KK737546.1, 1120580, 1120870, -, 291, 0; cds-WP _032429604.1, NZ_KK737546.1, 1120863, 1121033, -, 171, 0; cds-WP_032429606.1, NZ_KK737546.1, 1121014, 1121481, -, 468, 0; cds-WP_032429607.1, NZ_KK737546.1, 1121740, 1121937, -, 198, 0; cds-WP_032429608.1, NZ_KK737546.1, 1121930, 1122235, -, 306, 0; cds-WP_077254738.1, NZ_KK737546.1, 1122226, 1122615, -, 390, 1; cds-AF52_RS25290, NZ_KK737546.1, 1122697, 1122954, -, 258, 0; cds-WP_001096658.1, NZ_KK737546.1, 1123182, 1123418, -, 237, 0; cds-WP_032429609.1, NZ_KK737546.1, 1123418, 1123792, -, 375, 0; cds-WP_050484278.1, NZ_KK737546.1, 1123792, 1124439, -, 648, 0; cds-WP _032429610.1, NZ_KK737546.1, 1124546, 1124806, -, 261, 0; cds-WP _032429612.1, NZ_KK737546.1, 1124803, 1125198, -, 396, 0; cds-AF52_RS0123495, NZ_KK737546.1, 1125195, 1125496, -, 302, 7; cds-WP_187079187.1, NZ_KK737546.1, 1125496, 1126911, -, 1416, 1; cds-WP_187079188.1, NZ_KK737546.1, 1126916, 1127767, -, 852, 0; cds-WP_004151298.1-2, NZ_KK737546.1, 1127808, 1127954, -, 147, 0; cds-WP _004141720.1-2, NZ_KK737546.1, 1128041, 1128361, -, 321, 4168; cds-WP _023284762.1, NZ_KK737546.1, 1128411, 1128626, -, 216, 1642; cds-WP _019704102.1, NZ_KK737546.1, 1128726, 1129358, +, 633, 234; cds-WP_074440723.1, NZ_KK737546.1, 1129864, 1129989, +, 126, 454; cds-WP_072001571.1, NZ_KK737546.1, 1129982, 1130188, +, 207, 1; cds-WP_032429616.1, NZ_KK737546.1, 1130270, 1130554, +, 285, 11; cds-AF52_RS27875, NZ_KK737546.1, 1130566, 1130764, +, 199, 0; cds-WP_014342891.1, NZ_KK737546.1, 1130734, 1131069, +, 336, 0; cds-WP _032429617.1, NZ_KK737546.1, 1131066, 1131689, +, 624, 1; cds-WP_032429618.1, NZ_KK737546.1, 1131686, 1132114, +, 429, 1407; cds-WP_032429619.1, NZ_KK737546.1, 1132111, 1132767, +, 657, 2; cds-WP _032429620.1, NZ_KK737546.1, 1132764, 1133933, +, 1170, 0; cds-WP_032429621.1, NZ_KK737546.1, 1134147, 1134491, +, 345, 0; cds-WP _072001572.1, NZ_KK737546.1, 1134484, 1134678, +, 195, 0; cds-WP_032429622.1, NZ_KK737546.1, 1134675, 1134866, +, 192, 0; cds-WP_032429623.1, NZ_KK737546.1, 1134863, 1135081, +, 219, 0; cds-WP_019725112.1, NZ_KK737546.1, 1135085, 1135327, +, 243, 0; cds-WP_032429624.1, NZ_KK737546.1, 1135375, 1136637, +, 1263, 1418; cds-AF52_RS0123510, NZ_KK737546.1, 1136676, 1136903, +, 228, 0; cds-WP _004145468.1, NZ_KK737546.1, 1136878, 1137687, -, 810, 0; cds-WP_004175494.1, NZ_KK737546.1, 1137699, 1138994, -, 1296, 750; cds-WP _032429625.1, NZ_KK737546.1, 1139298, 1140224, -, 927, 5673; cds-WP _002910717.1, NZ_KK737546.1, 1140323, 1140799, +, 477, 9689; cds-WP_004148802.1, NZ_KK737546.1, 1140849, 1142492, -, 1644, 605742; cds-WP _002910720.1, NZ_KK737546.1, 1142776, 1143669, +, 894, 327; cds-WP _004175491.1, NZ_KK737546.1, 1143675, 1144394, -, 720, 0; cds-WP _004148803.1, NZ_KK737546.1, 1144391, 1145266, -, 876, 0; cds-WP_004148804.1, NZ_KK737546.1, 1145263, 1146549, -, 1287, 0; cds-WP_004175489.1, NZ_KK737546.1, 1146559, 1147473, -, 915, 0; cds-WP_004189329.1, NZ_KK737546.1, 1147583, 1148641, -, 1059, 0; cds-WP _004225356.1, NZ_KK737546.1, 1148931, 1149077, +, 147, 0; cds-WP_024622740.1, NZ_KK737546.1, 1149129, 1151423, -, 2295, 3; cds-WP_021440352.1, NZ_KK737546.1, 1151452, 1152660, -, 1209, 0; cds-WP_004145486.1, NZ_KK737546.1, 1152688, 1154019, -, 1332, 84; cds-WP_004211070.1, NZ_KK737546.1, 1154045, 1155034, -, 990, 0; cds-WP_002910764.1, NZ_KK737546.1, 1155128, 1156069, -, 942, 0; cds-WP _004148810.1, NZ_KK737546.1, 1156246, 1156389, +, 144, 0; cds-WP_004145488.1, NZ_KK737546.1, 1156687, 1156809, +, 123, 0; cds-WP _024622741.1, NZ_KK737546.1, 1156847, 1157647, -, 801, 0; cds-WP _032425076.1, NZ_KK737546.1, 1157640, 1158626, -, 987, 0; cds-WP _002910830.1, NZ_KK737546.1, 1159065, 1159811, -, 747, 14; cds-WP _008804319.1, NZ_KK737546.1, 1159828, 1160643, -, 816, 0; cds-WP_004184646.1, NZ_KK737546.1, 1160640, 1161569, -, 930, 0; cds-WP _004211064.1, NZ_KK737546.1, 1161563, 1163002, -, 1440, 0; cds-AF52_RS26335, NZ_KK737546.1, 1163005, 1163203, -, 199, 0; cds-WP _004175476.1, NZ_KK737546.1, 1163388, 1165133, +, 1746, 440; cds-WP_023328505.1, NZ_KK737546.1, 1165384, 1167504, +, 2121, 2019; cds-WP_002910846.1, NZ_KK737546.1, 1167546, 1168157, -, 612, 7558; cds-WP_002910883.1, NZ_KK737546.1, 1168333, 1169247, +, 915, 10; cds-WP _002910885.1, NZ _KK737546.1, 1169340, 1171073, +, 1734, 12694; cds-WP_002910887.1, NZ_KK737546.1, 1171090, 1172160, -, 1071, 43034; cds-WP _004175465.1, NZ_KK737546.1, 1172170, 1173468, -, 1299, 139371; cds-WP _032409324.1, NZ_KK737546.1, 1173567, 1173713, -, 147, 0; cds-WP_032429737.1, NZ_KK737546.1, 1173790, 1175322, +, 1533, 46909; cds-WP_002910890.1, NZ_KK737546.1, 1175397, 1176116, -, 720, 10350; cds-WP_004151442.1, NZ_KK737546.1, 1176365, 1177915, +, 1551, 24920; cds-WP_002910894.1, NZ _KK737546.1, 1178044, 1178574, +, 531, 2129; cds-WP_002910895.1, NZ _KK737546.1, 1178725, 1179069, +, 345, 0; cds-WP_021440363.1, NZ_KK737546.1, 1179076, 1179522, -, 447, 19; cds-WP _004180422.1, NZ_KK737546.1, 1179617, 1180276, -, 660, 5801; cds-WP _002910898.1, NZ_KK737546.1, 1180293, 1180574, -, 282, 28813; cds-WP_004180423.1, NZ_KK737546.1, 1180701, 1181399, +, 699, 36220; cds-WP_002910900.1, NZ_KK737546.1, 1181423, 1182235, +, 813, 287591; cds-WP _002910902.1, NZ_KK737546.1, 1182239, 1182508, +, 270, 186844; cds-WP_187079189.1, NZ_KK737546.1, 1182586, 1183701, -, 1116, 1986; cds-WP _002910904.1, NZ_KK737546.1, 1183783, 1185468, -, 1686, 14793; cds-WP_002910905.1, NZ _KK737546.1, 1185674, 1186255, -, 582, 15720; cds-WP _004151443.1, NZ_KK737546.1, 1186294, 1186989, -, 696, 14787; cds-WP_004175456.1, NZ_KK737546.1, 1187135, 1189045, -, 1911, 20175; cds-WP_002910910.1, NZ_KK737546.1, 1189176, 1189520, +, 345, 19090; cds-WP_004145519.1, NZ_KK737546.1, 1189521, 1189706, -, 186, 2372; cds-WP_004145520.1, NZ_KK737546.1, 1189795, 1191150, +, 1356, 6621; cds-WP_004180429.1, NZ_KK737546.1, 1191154, 1191732, +, 579, 12; cds-WP_004151445.1, NZ_KK737546.1, 1191895, 1193259, +, 1365, 51226; cds-WP _024622747.1, NZ_KK737546.1, 1193442, 1195025, +, 1584, 8543; cds-WP_004145524.1, NZ_KK737546.1, 1195059, 1196618, -, 1560, 56105; cds-WP_002910917.1, NZ_KK737546.1, 1197065, 1198036, +, 972, 174726; cds-WP _002910918.1, NZ _KK737546.1, 1198093, 1198893, +, 801, 286296; cds-WP_004145526.1, NZ _KK737546.1, 1198906, 1199757, +, 852, 161413; cds-WP_002910920.1, NZ _KK737546.1, 1199820, 1200278, +, 459, 19088; cds-WP _009484665.1, NZ_KK737546.1, 1200697, 1201263, +, 567, 1178; cds-WP _002910922.1, NZ_KK737546.1, 1201267, 1202061, -, 795, 1; cds-WP_002910924.1, NZ_KK737546.1, 1202123, 1203871, -, 1749, 0; cds-WP_001062678.1, NZ_KK737546.1, 1204059, 1204268, -, 210, 67103; cds-WP _071526670.1, NZ_KK737546.1, 1204351, 1204425, -, 75, 23353; cds-WP_002911374.1, NZ_KK737546.1, 1205016, 1205306, -, 291, 340; cds-WP _002911375.1, NZ_KK737546.1, 1205381, 1205524, -, 144, 191; cds-WP_004145533.1, NZ_KK737546.1, 1205684, 1205923, +, 240, 28223; cds-WP _002911378.1, NZ_KK737546.1, 1205980, 1206771, -, 792, 25836; cds-WP_072200054.1, NZ_KK737546.1, 1206898, 1208319, +, 1422, 1375; cds-WP _002911381.1, NZ_KK737546.1, 1208385, 1209269, -, 885, 274443; cds-WP _004145536.1, NZ_KK737546.1, 1209505, 1211553, -, 2049, 169503; cds-WP_002911385.1, NZ_KK737546.1, 1211573, 1212250, -, 678, 31752; cds-WP_002911386.1, NZ_KK737546.1, 1212348, 1212845, -, 498, 12488; cds-WP_002911387.1, NZ_KK737546.1, 1212978, 1214261, +, 1284, 40762; cds-WP _002911388.1, NZ_KK737546.1, 1214230, 1216863, +, 2634, 160357; cds-WP_032429627.1, NZ_KK737546.1, 1216988, 1218421, +, 1434, 1382; cds-WP _002911393.1, NZ_KK737546.1, 1218537, 1218776, +, 240, 699; cds-WP _002911395.1, NZ _KK737546.1, 1218874, 1219065, +, 192, 0; cds-WP_009484457.1, NZ_KK737546.1, 1219062, 1219715, -, 654, 357; cds-WP _014907334.1, NZ _KK737546.1, 1219872, 1220840, +, 969, 21380; cds-WP _002911398.1, NZ_KK737546.1, 1220945, 1221088, +, 144, 1; cds-WP _002911404.1, NZ_KK737546.1, 1221724, 1222062, -, 339, 13509; cds-WP_032429628.1, NZ_KK737546.1, 1222079, 1222948, -, 870, 873; cds-WP_004151448.1, NZ_KK737546.1, 1222952, 1223326, -, 375, 2692; cds-WP _002911406.1, NZ_KK737546.1, 1223439, 1223669, +, 231, 3481; cds-WP_002911407.1, NZ_KK737546.1, 1223748, 1224407, +, 660, 13413; cds-WP _004151449.1, NZ_KK737546.1, 1224411, 1226471, -, 2061, 17014; cds-WP_004180434.1, NZ_KK737546.1, 1226592, 1227251, -, 660, 26; cds-WP_004184662.1, NZ_KK737546.1, 1227391, 1227738, -, 348, 686; cds-WP_002911418.1, NZ_KK737546.1, 1227883, 1229061, +, 1179, 2243; cds-WP_002911423.1, NZ_KK737546.1, 1229119, 1229760, -, 642, 17355; cds-WP _002911425.1, NZ _KK737546.1, 1229799, 1231610, -, 1812, 16249; cds-WP _002911427.1, NZ _KK737546.1, 1231833, 1233308, -, 1476, 198694; cds-WP _004145554.1, NZ_KK737546.1, 1233319, 1233468, -, 150, 3425; cds-WP_004145555.1, NZ_KK737546.1, 1233628, 1234533, +, 906, 91081; cds-WP_002911440.1, NZ_KK737546.1, 1234659, 1236101, +, 1443, 265884; cds-WP_032429629.1, NZ_KK737546.1, 1236142, 1237116, -, 975, 7717; cds-WP_004148858.1, NZ_KK737546.1, 1237234, 1238553, -, 1320, 46997; cds-WP _004180437.1, NZ_KK737546.1, 1238569, 1239513, -, 945, 32337; cds-WP _002911449.1, NZ_KK737546.1, 1239592, 1240344, +, 753, 7489; cds-WP_004212745.1, NZ_KK737546.1, 1240344, 1241129, +, 786, 1; cds-WP _004148860.1, NZ_KK737546.1, 1241193, 1242203, -, 1011, 466; cds-WP_002911454.1, NZ_KK737546.1, 1242212, 1242823, -, 612, 284; cds-WP_002911456.1, NZ_KK737546.1, 1242903, 1243424, -, 522, 152; cds-WP_002911459.1, NZ_KK737546.1, 1243459, 1244199, -, 741, 25191; cds-WP _002911477.1, NZ_KK737546.1, 1244227, 1244670, -, 444, 5540; cds-WP_002911479.1, NZ_KK737546.1, 1244672, 1246459, -, 1788, 175527; cds-WP_004145564.1, NZ_KK737546.1, 1246727, 1247293, +, 567, 7154; cds-WP _009307531.1, NZ_KK737546.1, 1247290, 1248108, +, 819, 7934; cds-WP _002911484.1, NZ_KK737546.1, 1248161, 1248556, +, 396, 1463; cds-WP_004175417.1, NZ_KK737546.1, 1248596, 1249339, +, 744, 12261; cds-WP _032429630.1, NZ_KK737546.1, 1249336, 1250340, +, 1005, 1599; cds-WP _032429631.1,
NZ _KK737546.1, 1250422, 1251165, -, 744, 3287; cds-WP _015958659.1, NZ_KK737546.1, 1251242, 1251811, -, 570, 0; cds-WP _004151452.1, NZ _KK737546.1, 1252047, 1253780, +, 1734, 56470; cds-WP _002911497.1, NZ_KK737546.1, 1253842, 1254981, -, 1140, 167; cds-WP _021440394.1, NZ_KK737546.1, 1254986, 1256569, -, 1584, 0; cds-WP_002911500.1, NZ_KK737546.1, 1256923, 1257351, +, 429, 3; cds-WP _004175414.1, NZ_KK737546.1, 1257375, 1258799, -, 1425, 20; cds-WP _002911505.1, NZ_KK737546.1, 1258774, 1259562, -, 789, 2; cds-WP _002911507.1, NZ_KK737546.1, 1259724, 1260704, -, 981, 0; cds-WP_004184670.1, NZ_KK737546.1, 1260719, 1262233, -, 1515, 1; cds-WP_009484443.1, NZ_KK737546.1, 1262296, 1263276, -, 981, 0; cds-WP_160540596.1, NZ_KK737546.1, 1263638, 1263736, +, 99, 0; cds-WP _004175413.1, NZ_KK737546.1, 1264168, 1264677, +, 510, 2; cds-WP_004148869.1, NZ_KK737546.1, 1264748, 1264906, -, 159, 4; cds-WP_002911522.1, NZ_KK737546.1, 1264972, 1266483, +, 1512, 353; cds-WP_002911524.1, NZ_KK737546.1, 1266521, 1266772, -, 252, 164; cds-WP _021440395.1, NZ_KK737546.1, 1266923, 1268344, +, 1422, 6088; cds-WP _021440396.1, NZ_KK737546.1, 1268394, 1269032, -, 639, 0; cds-AF52_RS0123570, NZ_KK737546.1, 1269204, 1269384, +, 181, 0; cds-WP _004141101.1, NZ_KK737546.1, 1269442, 1269939, +, 498, 25074; cds-WP_024622766.1, NZ_KK737546.1, 1269975, 1270214, -, 240, 0; cds-WP_004151453.1, NZ_KK737546.1, 1270406, 1271617, +, 1212,5022; cds-WP _004141106.1, NZ_KK737546.1, 1271642, 1272310, -, 669, 2070; cds-WP _004180444.1, NZ_KK737546.1, 1272409, 1273290, -, 882, 16; cds-WP_002911537.1, NZ_KK737546.1, 1273958, 1274506, -, 549, 6841; cds-WP _002911538.1, NZ_KK737546.1, 1274564, 1276396, -, 1833, 43594; cds-WP _002911539.1, NZ_KK737546.1, 1276393, 1277049, -, 657, 9402; cds-WP_002911541.1, NZ_KK737546.1, 1277511, 1277735, +, 225, 162; cds-WP _032429632.1, NZ_KK737546.1, 1277803, 1278525, -, 723, 20744; cds-WP _024622767.1, NZ_KK737546.1, 1278843, 1279595, -, 753, 507; cds-WP_002911547.1, NZ_KK737546.1, 1279592, 1280260, -, 669, 80; cds-WP_021313403.1, NZ_KK737546.1, 1280276, 1281262, -, 987, 2162; cds-WP _004141135.1, NZ_KK737546.1, 1281358, 1282158, -, 801, 4051; cds-WP_004899206.1, NZ_KK737546.1, 1282359, 1283846, +, 1488, 91; cds-WP_004189223.1, NZ_KK737546.1, 1283888, 1284322, -, 435, 551; cds-WP_004180447.1, NZ_KK737546.1, 1284695, 1285318, +, 624, 6195; cds-WP _002911586.1, NZ_KK737546.1, 1285351, 1285542, -, 192, 7584; cds-WP _002911589.1, NZ_KK737546.1, 1285676, 1285897, +, 222, 0; cds-WP _013815099.1-26, NZ_KK737546.1, 1286063, 1287031, -, 969, 5522; cds-AF52_RS25350, NZ_KK737546.1, 1287063, 1287197, -, 135, 0; cds-WP _004184683.1, NZ_KK737546.1, 1287375, 1288286, +, 912, 44; cds-WP_024622769.1, NZ_KK737546.1, 1288261, 1288755, -, 495, 830; cds-WP_004153246.1, NZ_KK737546.1, 1288736, 1290136, -, 1401, 287; cds-WP _002911594.1, NZ_KK737546.1, 1290213, 1290920, -, 708, 195; cds-WP_004151461.1, NZ_KK737546.1, 1290963, 1291244, -, 282, 4; cds-WP _002911596.1, NZ_KK737546.1, 1291783, 1292928, +, 1146, 8269; cds-WP _004227143.1, NZ_KK737546.1, 1293381, 1294181, +, 801, 3769; cds-WP_009484428.1, NZ_KK737546.1, 1294767, 1295462, +, 696, 23; cds-WP_016946077.1, NZ_KK737546.1, 1295493, 1295690, +, 198, 99; cds-WP_022631352.1, NZ_KK737546.1, 1295840, 1296781, -, 942, 2807; cds-WP_004175385.1, NZ_KK737546.1, 1296860, 1297810, -, 951, 0; cds-WP _002911729.1, NZ_KK737546.1, 1297917, 1298834, -, 918, 0; cds-WP _004148971.1, NZ_KK737546.1, 1299334, 1300968, -, 1635, 173; cds-WP_022631351.1, NZ_KK737546.1, 1301560, 1302264, +, 705, 7162; cds-WP _004151466.1, NZ_KK737546.1, 1302261, 1303238, +, 978, 2230; cds-WP _004184763.1, NZ_KK737546.1, 1303359, 1304291, +, 933, 39119; cds-WP_032429633.1, NZ_KK737546.1, 1304202, 1305824, -, 1623, 0; cds-WP _004141189.1, NZ_KK737546.1, 1306097, 1306342, +, 246, 0; cds-WP _004148975.1, NZ_KK737546.1, 1306472, 1307926, +, 1455, 73771; cds-WP _004151469.1, NZ_KK737546.1, 1308292, 1309728, -, 1437, 2; cds-AF52_RS25355, NZ_KK737546.1, 1310050, 1310136, -, 87, 0; cds-WP_022631349.1, NZ_KK737546.1, 1310278, 1311381, +, 1104, 2; cds-WP _002911798.1, NZ_KK737546.1, 1311378, 1312349, -, 972, 570; cds-WP _004180471.1, NZ_KK737546.1, 1312556, 1313392, -, 837, 158; cds-WP_016532101.1, NZ_KK737546.1, 1313585, 1314160, -, 576, 0; cds-WP_022631348.1, NZ_KK737546.1, 1314292, 1315206, +, 915, 15; cds-WP _002911862.1, NZ_KK737546.1, 1315212, 1315547, -, 336, 0; cds-WP _002911866.1, NZ_KK737546.1, 1315554, 1315886, -, 333, 0; cds-WP_004144110.1, NZ_KK737546.1, 1315984, 1316112, +, 129, 0; cds-WP_016831855.1, NZ _KK737546.1, 1316109, 1318019, -, 1911, 19452; cds-WP_014907250.1, NZ_KK737546.1, 1318030, 1318491, -, 462, 1618; cds-WP_022631347.1, NZ_KK737546.1, 1318835, 1320154, +,1320,0; cds-WP_022631346.1, NZ_KK737546.1, 1320201, 1320941, +, 741, 0; cds-WP_032429739.1, NZ_KK737546.1, 1320901, 1321857, -, 957, 71; cds-WP_004175282.1, NZ_KK737546.1, 1321973, 1322362, +, 390, 572; cds-WP_002911879.1, NZ_KK737546.1, 1322384, 1323115, -, 732, 1; cds-WP _004200363.1, NZ_KK737546.1, 1323314, 1324423, +, 1110, 12582; cds-WP _004200364.1, NZ_KK737546.1, 1324466, 1325533, -, 1068, 11; cds-WP_021440419.1, NZ_KK737546.1, 1325561, 1326301, -, 741, 27; cds-WP_002911883.1, NZ_KK737546.1, 1326616, 1326939, -, 324, 777; cds-WP _032429634.1, NZ_KK737546.1, 1327107, 1328165, -, 1059, 513; cds-WP_021440421.1, NZ_KK737546.1, 1328264, 1328737, -, 474, 2019; cds-WP_004153104.1, NZ_KK737546.1, 1328913, 1330076, -, 1164, 519; cds-WP _032429635.1, NZ_KK737546.1, 1330257, 1331681, +, 1425, 72414; cds-WP _002912106.1, NZ _KK737546.1, 1331905, 1334007, -, 2103, 26; cds-WP_002912109.1, NZ_KK737546.1, 1334247, 1334558, +, 312, 10; cds-WP_004148994.1, NZ_KK737546.1, 1334740, 1336098, -, 1359, 10485; cds-WP _096335043.1, NZ_KK737546.1, 1336088, 1336150, -, 63, 1126; cds-WP _004175276.1, NZ_KK737546.1, 1336388, 1337278, -, 891, 32; cds-WP_004152513.1, NZ_KK737546.1, 1337317, 1338141, -, 825, 209; cds-WP_004899375.1, NZ_KK737546.1, 1338318, 1338368, +, 51, 0; cds-WP _002912152.1, NZ_KK737546.1, 1338514, 1339413, +, 900, 89; cds-WP _009307478.1, NZ_KK737546.1, 1339453, 1340757, +, 1305, 122; cds-WP _032429636.1, NZ _KK737546.1, 1340754, 1341815, +, 1062, 1060; cds-WP_004144133.1, NZ_KK737546.1, 1341812, 1342879, +, 1068, 87; cds-WP _002912227.1, NZ_KK737546.1, 1342879, 1343469, +, 591, 2; cds-WP _004175272.1, NZ_KK737546.1, 1343469, 1344206, +, 738, 11; cds-WP _004180497.1, NZ_KK737546.1, 1344188, 1344964, +, 777, 10733; cds-WP_032429637.1, NZ_KK737546.1, 1344958, 1345557, +, 600, 5907; cds-WP _004198893.1, NZ_KK737546.1, 1345750, 1346127, +, 378, 1926; cds-AF52_RS25360, NZ_KK737546.1, 1346124, 1346378, +, 255, 15; cds-AF52_RS16545, NZ_KK737546.1, 1351106, 1351364, +, 259, 1332; cds-AF52_RS16540, NZ_KK737546.1, 1351435, 1352232, +, 798, 160; cds-AF52_RS27905, NZ_KK737546.1, 1352265, 1352369, +, 105, 218; cds-AF52_RS0123600, NZ_KK737546.1, 1352352, 1353032, +, 681, 241; cds-AF52_RS0123605, NZ_KK737546.1, 1353102, 1353650, -, 549, 6014; cds-WP_013815099.1-27, NZ_KK737546.1, 1353685, 1354653, +, 969, 9667; cds-AF52_RS25365, NZ_KK737546.1, 1354708, 1355376, -, 669, 6059; cds-WP _072269281.1, NZ_KK737546.1, 1355472, 1356614, -, 1143, 15027; cds-WP_032429639.1, NZ_KK737546.1, 1356615, 1357508, -, 894, 172517; cds-WP_004890861.1, NZ_KK737546.1, 1357505, 1358659, -, 1155, 85872; cds-WP _004214006.1, NZ_KK737546.1, 1358675, 1360570, -, 1896, 183455; cds-WP _004175264.1, NZ_KK737546.1, 1360586, 1361326, -, 741, 32281; cds-WP_002912373.1, NZ_KK737546.1, 1361326, 1362093, -, 768, 20095; cds-WP _013815099.1-28, NZ_KK737546.1, 1362205, 1363173, +, 969, 6289; cds-WP _032429640.1, NZ _KK737546.1, 1364205, 1365209, +, 1005, 22305; cds-AF52_RS26960, NZ_KK737546.1, 1368345, 1368481, +, 137, 226; cds-AF52_RS26965, NZ_KK737546.1, 1368526, 1368645, -, 120, 56; cds-WP_032429643.1, NZ _KK737546.1, 1368690, 1368818, +, 129, 1; cds-WP _032429644.1, NZ_KK737546.1, 1369241, 1370407, -, 1167, 56381; cds-WP _004899416.1, NZ_KK737546.1, 1370571, 1371941, -, 1371, 110659; cds-WP _004180506.1, NZ_KK737546.1, 1371964, 1373379, -, 1416, 159418; cds-WP_020802781.1, NZ_KK737546.1, 1373607, 1375013, -, 1407, 1166312; cds-WP _013815099.1-29, NZ_KK737546.1, 1375211, 1376179, -, 969, 5890; cds-WP_020802783.1, NZ_KK737546.1, 1376246, 1377643, -, 1398, 1234; cds-WP_032429645.1, NZ_KK737546.1, 1377862, 1378497, -, 636, 0; cds-AF52_RS26980, NZ_KK737546.1, 1381542, 1381660, -, 119, 4226; cds-WP _072001573.1, NZ_KK737546.1, 1381682, 1382065, -, 384, 9545; cds-WP _032429646.1, NZ_KK737546.1, 1382073, 1383821, -, 1749, 61917; cds-WP_020801666.1, NZ_KK737546.1, 1383842, 1384609, -, 768, 67463; cds-WP_020801633.1, NZ_KK737546.1, 1384619, 1385788, -, 1170, 94805; cds-WP_032429647.1, NZ_KK737546.1, 1385800, 1386888, -, 1089, 28447; cds-WP_032429746.1, NZ_KK737546.1, 1386888, 1388180, -, 1293, 3765; cds-WP_020801632.1, NZ_KK737546.1, 1388137, 1389291, -, 1155, 5530; cds-WP _020801659.1, NZ_KK737546.1, 1389302, 1390468, -, 1167, 124241; cds-WP_020801642.1, NZ_KK737546.1, 1390472, 1391422, -, 951, 61904; cds-WP_013815099.1-30, NZ_KK737546.1, 1391484, 1392452, +, 969, 20438; cds-WP _032429648.1, NZ_KK737546.1, 1392641, 1394812, -, 2172, 137210; cds-WP_072001574.1, NZ_KK737546.1, 1394831, 1395262, -, 432, 64498; cds-WP _020801619.1, NZ_KK737546.1, 1395269, 1396402, -, 1134, 180323; cds-WP_032429649.1, NZ_KK737546.1, 1396548, 1397981, -, 1434, 72155; cds-WP_032429650.1, NZ_KK737546.1, 1398272, 1398394, +, 123, 12; cds-WP _020806188.1-2, NZ_KK737546.1, 1398736, 1399716, -, 981, 6892; cds-WP _004184823.1, NZ_KK737546.1, 1400141, 1400770, -, 630, 1286; cds-WP_001741945.1, NZ_KK737546.1, 1401163, 1402053, -, 891, 41417; cds-WP _004151147.1, NZ_KK737546.1, 1402818, 1404401, +, 1584, 2; cds-WP _032412678.1, NZ_KK737546.1, 1404896, 1406743, -, 1848, 423; cds-WP _004151145.1, NZ_KK737546.1, 1406774, 1407355, -, 582, 21854; cds-WP_002912442.1, NZ_KK737546.1, 1407446, 1408087, -, 642, 66543; cds-WP_032429651.1, NZ_KK737546.1, 1408202, 1409050, -, 849, 7; cds-WP_032429652.1, NZ_KK737546.1, 1409188, 1410540, +, 1353, 6; cds-WP _032429747.1, NZ_KK737546.1, 1410830, 1412686, +, 1857, 26756; cds-WP_032429653.1, NZ_KK737546.1, 1412701, 1413402, +, 702, 16176; cds-WP _032409828.1, NZ_KK737546.1, 1413640, 1413780, +, 141, 0; cds-WP_004899454.1, NZ_KK737546.1, 1413896, 1414507, +, 612, 3; cds-WP _004149053.1, NZ _KK737546.1, 1414781, 1416055, -, 1275, 0; cds-WP _032429654.1, NZ_KK737546.1, 1416356, 1417594, +, 1239, 8; cds-WP _004149055.1, NZ _KK737546.1, 1417594, 1420716, +, 3123, 6029; cds-WP _032429655.1, NZ_KK737546.1, 1420717, 1423794, +, 3078, 55; cds-WP_032429656.1, NZ_KK737546.1, 1423796, 1425211, +, 1416, 0; cds-WP_004149058.1, NZ_KK737546.1, 1425208, 1426686, +, 1479, 946; cds-WP _004175198.1, NZ_KK737546.1, 1426683, 1427405, +, 723, 582; cds-WP _004144192.1, NZ _KK737546.1, 1427724, 1429085, +, 1362, 418; cds-WP_004151134.1, NZ _KK737546.1, 1429328, 1430224, +, 897,1; cds-WP_032429657.1, NZ_KK737546.1, 1430465, 1431238, -, 774, 11; cds-WP _032429748.1, NZ_KK737546.1, 1431249, 1432112, -, 864, 3; cds-WP _004200464.1, NZ_KK737546.1, 1432084, 1432989, -, 906, 10; cds-WP _004149062.1, NZ_KK737546.1, 1432986, 1434023, -, 1038, 15; cds-WP_004180553.1, NZ_KK737546.1, 1434020, 1435642, -, 1623, 17; cds-WP_002912648.1, NZ_KK737546.1, 1435762, 1436916, -, 1155, 8; cds-WP _032429658.1, NZ_KK737546.1, 1437214, 1437876, -, 663, 0; cds-WP _016529109.1, NZ_KK737546.1, 1437925, 1439202, -, 1278, 68; cds-WP_032429659.1, NZ_KK737546.1, 1439273, 1440736, -, 1464, 0; cds-WP _004144197.1, NZ_KK737546.1, 1440746, 1442113, -, 1368, 1; cds-WP_004144198.1, NZ_KK737546.1, 1442320,1443261, +, 942, 32; cds-WP_032429660.1, NZ_KK737546.1, 1443284, 1444285, -, 1002, 5121; cds-WP_004180592.1, NZ_KK737546.1, 1444540, 1445289, +, 750, 0; cds-WP_023285368.1, NZ_KK737546.1, 1445316, 1446923, +, 1608, 33832; cds-WP _004175116.1, NZ_KK737546.1, 1447005, 1448288, +, 1284, 1333; cds-WP _002912704.1, NZ_KK737546.1, 1448347, 1449399, -, 1053, 97; cds-WP _002912707.1, NZ_KK737546.1, 1449547, 1450347, -, 801, 4869; cds-WP _004151131.1, NZ_KK737546.1, 1450344, 1451117, -, 774, 21; cds-WP _009307427.1, NZ_KK737546.1, 1451435, 1451857, +, 423, 112; cds-WP _004175111.1, NZ_KK737546.1, 1451854, 1452768, -, 915, 29; cds-WP_009307426.1, NZ_KK737546.1, 1452889, 1454700, +, 1812, 865; cds-WP _009307425.1, NZ_KK737546.1, 1454703, 1456046, +, 1344, 0; cds-WP _004149074.1,
NZ_KK737546.1, 1456036, 1456935, -, 900, 10; cds-WP_002912749.1, NZ_KK737546.1, 1457119, 1457436, +, 318, 1513; cds-WP _072269284.1, NZ_KK737546.1, 1457408, 1457872, +, 465, 14; cds-WP_032429661.1, NZ_KK737546.1, 1457869, 1458978, -, 1110, 10057; cds-WP_002912753.1, NZ_KK737546.1, 1459132, 1461165, +, 2034, 89294; cds-WP_002912756.1, NZ_KK737546.1, 1461280, 1461750, -, 471, 59724; cds-WP _004144215.1, NZ_KK737546.1, 1461798, 1462517, -, 720, 25949; cds-WP _002912760.1, NZ_KK737546.1, 1462511, 1464199, -, 1689, 16763; cds-WP _002912762.1, NZ_KK737546.1, 1464429, 1464542, +, 114, 0; cds-WP _022631308.1, NZ_KK737546.1, 1464517, 1465254, -, 738, 1070; cds-WP_022631307.1, NZ_KK737546.1, 1465238, 1466185, -, 948, 0; cds-WP _023285369.1, NZ_KK737546.1, 1466178, 1467335, -, 1158, 4677; cds-WP_002912823.1, NZ_KK737546.1, 1467343, 1468260, -, 918, 6378; cds-WP_009307419.1, NZ_KK737546.1, 1468397, 1470694, -, 2298, 44745; cds-WP _002912827.1, NZ_KK737546.1, 1470889, 1472634, +, 1746, 65919; cds-WP_002912829.1, NZ_KK737546.1, 1472720, 1473274, +, 555, 4864; cds-WP _004180615.1, NZ_KK737546.1, 1473497, 1474447, -, 951, 39564; cds-WP _002912831.1, NZ_KK737546.1, 1474636, 1475223, -, 588, 0; cds-WP_009307418.1, NZ_KK737546.1, 1475378, 1475947, +, 570, 4; cds-WP_032429662.1, NZ_KK737546.1, 1475981, 1477420, -, 1440, 55701; cds-WP _022631304.1, NZ_KK737546.1, 1477692, 1479086, -, 1395, 28384; cds-WP _032429663.1, NZ_KK737546.1, 1479102, 1480964, -, 1863, 11212; cds-WP _002912842.1, NZ_KK737546.1, 1481107, 1481940, -, 834, 510; cds-WP _004175080.1, NZ_KK737546.1, 1482096, 1483460, -, 1365, 0; cds-WP _002912862.1, NZ_KK737546.1, 1483817, 1484749, -, 933, 75; cds-WP _002912865.1, NZ_KK737546.1, 1485107, 1485505, +, 399, 2; cds-WP_002912867.1, NZ_KK737546.1, 1485502, 1486197, +, 696, 0; cds-WP_002912869.1, NZ_KK737546.1, 1486325, 1487209, +, 885, 7; cds-WP_004211527.1, NZ_KK737546.1, 1487485,1488201, +, 717, 38479; cds-WP_002912871.1, NZ_KK737546.1, 1488272, 1489282, -, 1011, 191511; cds-WP_004175074.1, NZ_KK737546.1, 1489298, 1490818, -, 1521, 314069; cds-WP_002912878.1, NZ_KK737546.1, 1490937, 1491935, -, 999, 392077; cds-WP_002912919.1, NZ_KK737546.1, 1492232, 1493254, -, 1023, 79683; cds-WP_021440476.1, NZ_KK737546.1, 1493410, 1494567, -, 1158, 19830; cds-WP _002912924.1, NZ_KK737546.1, 1494583, 1495251, -, 669, 79155; cds-WP _004151123.1, NZ_KK737546.1, 1495612, 1496445, +, 834, 8906; cds-WP _032429664.1, NZ_KK737546.1, 1496516, 1498489, -, 1974, 40906; cds-WP_002912929.1, NZ_KK737546.1, 1498988, 1500457, -, 1470, 40399; cds-WP_002912932.1, NZ_KK737546.1, 1500636, 1501502, -, 867, 0; cds-WP_002912935.1, NZ_KK737546.1, 1501768, 1502817, +, 1050, 0; cds-WP_002912937.1, NZ_KK737546.1, 1502890, 1503744, +, 855, 120; cds-WP _021440480.1, NZ_KK737546.1, 1503795, 1505489, -, 1695, 28918; cds-WP _002912941.1, NZ_KK737546.1, 1505506, 1506444, -, 939, 21507; cds-WP _032428355.1, NZ_KK737546.1, 1506445, 1507575, -, 1131, 96; cds-WP _032429665.1, NZ_KK737546.1, 1507911, 1509092, +, 1182, 1276; cds-WP_004175066.1, NZ_KK737546.1, 1509132, 1509386, -, 255, 1742; cds-WP _002912951.1, NZ_KK737546.1, 1509534, 1510106, +, 573, 66985; cds-WP_002912952.1, NZ_KK737546.1, 1510177, 1511367, -, 1191, 23093; cds-WP_004200498.1, NZ_KK737546.1, 1511575, 1513041, +, 1467, 1950; cds-WP_021463904.1, NZ_KK737546.1, 1513162, 1514139, +, 978, 1; cds-WP _072269286.1, NZ_KK737546.1, 1514177, 1514890, +, 714, 21510; cds-WP_002912967.1, NZ_KK737546.1, 1515317, 1515886, +, 570, 50408; cds-WP_021440487.1, NZ _KK737546.1, 1516075, 1517637, +, 1563, 181352; cds-WP _032429666.1, NZ_KK737546.1, 1517706, 1519511, +, 1806, 49; cds-WP_004200501.1, NZ_KK737546.1, 1519521, 1520615, +, 1095, 126; cds-WP _004149131.1, NZ_KK737546.1, 1520615, 1521640, +, 1026, 7; cds-WP _004151118.1, NZ_KK737546.1, 1521642, 1523231, +, 1590, 256; cds-WP _002912974.1, NZ_KK737546.1, 1523235, 1523579, -, 345, 2413; cds-WP_002912975.1, NZ_KK737546.1, 1523911, 1525107, -, 1197, 186; cds-WP _002912977.1, NZ_KK737546.1, 1525104, 1525823, -, 720, 1807; cds-WP _004144263.1, NZ_KK737546.1, 1525971, 1527728, +, 1758, 730; cds-WP_002912979.1, NZ_KK737546.1, 1527865, 1528149, +, 285, 18731; cds-WP _004189004.1, NZ_KK737546.1, 1528208, 1529095, -, 888, 27; cds-AF52_RS0123640, NZ_KK737546.1, 1529200, 1530485, +, 1286, 0; cds-WP_024622810.1, NZ_KK737546.1, 1530504, 1531100, -, 597, 0; cds-WP_002912986.1, NZ_KK737546.1, 1531183, 1531944, +, 762, 485; cds-WP _004144267.1, NZ_KK737546.1, 1531964, 1532971, -, 1008, 17838; cds-WP _004195346.1, NZ_KK737546.1, 1533158, 1533385, +, 228, 120; cds-WP_002912990.1, NZ_KK737546.1, 1533404, 1535164, +, 1761, 18829; cds-WP _032429667.1, NZ_KK737546.1, 1535502, 1536611, +, 1110, 5020; cds-WP _032428356.1, NZ_KK737546.1, 1536786, 1537277, +, 492, 200878; cds-WP _002912994.1, NZ_KK737546.1, 1537344, 1538978, -, 1635, 14569; cds-AF52_RS0123645, NZ_KK737546.1, 1539025, 1539722, -, 698, 16586; cds-WP_020324457.1, NZ_KK737546.1, 1540115, 1541275, +, 1161, 5570; cds-WP_002912996.1, NZ_KK737546.1, 1541238, 1542674, -, 1437, 9666; cds-WP_004184919.1, NZ_KK737546.1, 1542823, 1544466, -, 1644, 45; cds-WP_016530253.1, NZ_KK737546.1, 1544541, 1545194, -, 654, 0; cds-WP_021440495.1, NZ_KK737546.1, 1545194, 1546258, -, 1065, 2339; cds-WP _032429668.1, NZ_KK737546.1, 1546332, 1547384, -, 1053, 1072; cds-WP_032429669.1, NZ_KK737546.1, 1547488, 1548582, -, 1095, 2203612; cds-WP_002913006.1, NZ_KK737546.1, 1549354, 1552011, +, 2658, 30889; cds-WP _002913007.1, NZ_KK737546.1, 1552027, 1552677, +, 651, 16362; cds-WP_032429670.1, NZ_KK737546.1, 1552723, 1555563, -, 2841, 139781; cds-WP _032429671.1, NZ_KK737546.1, 1555695, 1558328, -, 2634, 143101; cds-WP _002913014.1, NZ_KK737546.1, 1558475, 1559203, +, 729, 703; cds-WP _002913016.1, NZ_KK737546.1, 1559548, 1561833, +, 2286, 63733; cds-WP _004140835.1, NZ_KK737546.1, 1561935, 1563065, +, 1131, 153478; cds-WP _002913017.1, NZ_KK737546.1, 1563065, 1563319, +, 255, 22768; cds-WP _032429672.1, NZ_KK737546.1, 1563783, 1564853, -, 1071, 755865; cds-WP_002913019.1, NZ_KK737546.1, 1564863, 1566209, -, 1347, 553531; cds-WP _004214634.1, NZ_KK737546.1, 1566481, 1568103, +, 1623, 0; cds-WP _015958752.1, NZ_KK737546.1, 1568093, 1569352, +, 1260, 0; cds-WP_004184966.1, NZ_KK737546.1, 1569349, 1570527, +, 1179, 115; cds-WP_032428357.1, NZ_KK737546.1, 1570549, 1571352, -, 804, 9; cds-WP_002913045.1, NZ_KK737546.1, 1571367, 1572656, -, 1290, 0; cds-WP_004175029.1, NZ_KK737546.1, 1572699, 1573904, -, 1206, 0; cds-WP_002913046.1, NZ_KK737546.1, 1573915, 1574700, -, 786, 0; cds-WP _004184973.1, NZ_KK737546.1, 1574905, 1576101, -, 1197, 46070; cds-WP _019705497.1, NZ_KK737546.1, 1576204, 1576746, -, 543, 11351; cds-WP _024622815.1, NZ_KK737546.1, 1577021, 1577962, -, 942, 221; cds-WP _032429673.1, NZ_KK737546.1, 1578100, 1578525, +, 426, 0; cds-WP _021440500.1, NZ_KK737546.1, 1578581, 1579957, -, 1377, 3735; cds-WP _021440501.1, NZ_KK737546.1, 1579954, 1580919, -, 966, 47415; cds-WP_002913098.1, NZ_KK737546.1, 1580919, 1581776, -, 858, 3254; cds-WP _021440502.1, NZ_KK737546.1, 1581791, 1582549, -, 759, 118; cds-WP _002913103.1, NZ_KK737546.1, 1582546, 1584216, -, 1671, 21; cds-WP_021440503.1, NZ_KK737546.1, 1584299, 1585588, -, 1290, 6; cds-WP_004184987.1, NZ_KK737546.1, 1585724, 1586185, -, 462, 7699; cds-WP_004214593.1, NZ_KK737546.1, 1586243, 1587163, +, 921, 14; cds-WP _004151107.1, NZ_KK737546.1, 1587218, 1588675, -, 1458, 64799; cds-WP_032429674.1, NZ_KK737546.1, 1588682, 1590211, -, 1530, 326802; cds-WP _032429675.1, NZ_KK737546.1, 1590381, 1592222, -, 1842, 205426; cds-WP _002913148.1, NZ_KK737546.1, 1592219, 1592521, -, 303, 36962; cds-WP_002913150.1, NZ_KK737546.1, 1592518, 1593072, -, 555, 146159; cds-WP_002913152.1, NZ_KK737546.1, 1593084, 1593626, -, 543, 245938; cds-WP_002913156.1, NZ_KK737546.1, 1593641, 1594618, -, 978, 117528; cds-WP_023285394.1, NZ_KK737546.1, 1594615, 1597341, -, 2727, 317211; cds-WP_002913158.1, NZ_KK737546.1, 1597374, 1598711, -, 1338, 487311; cds-WP _002913160.1, NZ_KK737546.1, 1598708, 1599208, -, 501, 62403; cds-WP _002913162.1, NZ_KK737546.1, 1599211, 1601019, -, 1809, 806231; cds-WP_002913178.1, NZ_KK737546.1, 1601106, 1601780, -, 675, 297635; cds-WP_002913181.1, NZ_KK737546.1, 1601796, 1602242, -, 447, 58633; cds-WP_021440508.1, NZ _KK737546.1, 1602894, 1603820, -, 927, 8949; cds-WP_002913185.1, NZ_KK737546.1, 1604693, 1605910, +, 1218, 108565; cds-WP_002913188.1, NZ_KK737546.1, 1605992, 1606591, +, 600, 92061; cds-WP _032428358.1, NZ_KK737546.1, 1606636, 1608468, -, 1833, 42684; cds-WP _019705510.1, NZ_KK737546.1, 1608545, 1609204, -, 660, 9838; cds-WP _002913193.1, NZ_KK737546.1, 1609216, 1609710, -, 495, 3368; cds-WP _002913194.1, NZ_KK737546.1, 1609798, 1610253, -, 456, 43; cds-WP_002913195.1, NZ_KK737546.1, 1610591, 1611793, +, 1203, 484395; cds-WP_002913196.1, NZ_KK737546.1, 1611994, 1614141, +, 2148, 530303; cds-WP_032429676.1, NZ_KK737546.1, 1614234, 1614785, -, 552, 24658; cds-WP _004174991.1, NZ_KK737546.1, 1614843, 1615394, -, 552, 33259; cds-WP _004180746.1, NZ_KK737546.1, 1615457, 1616095, -, 639, 13236; cds-WP_004174988.1, NZ_KK737546.1, 1616276, 1616905, +, 630, 0; cds-WP _002913200.1, NZ_KK737546.1, 1616971, 1617333, +, 363, 3161; cds-WP _002913203.1, NZ_KK737546.1, 1617352, 1618245, +, 894, 6884; cds-WP_004217247.1, NZ_KK737546.1, 1618225, 1618341, -, 117, 0; cds-WP_032429677.1, NZ_KK737546.1, 1618461, 1618988, +, 528, 72; cds-WP_002913206.1, NZ_KK737546.1, 1618994, 1619767, -, 774, 1628; cds-WP_002913207.1, NZ_KK737546.1, 1619775, 1620491, -, 717, 64192; cds-WP_002913208.1, NZ_KK737546.1, 1620488, 1621174, -, 687, 7410; cds-WP _002913209.1, NZ_KK737546.1, 1621239, 1622021, -, 783, 89476; cds-WP _002913210.1, NZ_KK737546.1, 1622289, 1623071, -, 783, 68617; cds-WP_004140952.1, NZ_KK737546.1, 1623117, 1623278, +, 162, 10154; cds-WP _014599227.1, NZ_KK737546.1, 1623359, 1623928, -, 570, 3; cds-WP_032429678.1, NZ_KK737546.1, 1624064, 1625581, -, 1518, 49321; cds-WP _002913214.1, NZ_KK737546.1, 1625615, 1626103, -, 489, 3500; cds-WP _004151102.1, NZ_KK737546.1, 1626320, 1627009, -, 690, 882; cds-WP _021440515.1, NZ_KK737546.1, 1626999, 1628267, -, 1269, 19796; cds-WP_002913220.1, NZ_KK737546.1, 1628343, 1629263, -, 921, 118618; cds-WP _002913221.1, NZ_KK737546.1, 1629449, 1630108, -, 660, 10420; cds-WP _002913226.1, NZ_KK737546.1, 1630135, 1630947, -, 813, 5197; cds-WP_002913227.1, NZ_KK737546.1, 1630947, 1631960, -, 1014, 4666; cds-WP _021440517.1, NZ_KK737546.1, 1632024, 1633160, -, 1137, 11171; cds-WP_032429679.1, NZ_KK737546.1, 1633271, 1634248, +, 978, 23; cds-WP_004149211.1, NZ_KK737546.1, 1634335, 1635510, -, 1176, 911; cds-WP_002913291.1, NZ_KK737546.1, 1635720, 1636940, -, 1221, 3400; cds-WP_032429751.1, NZ_KK737546.1, 1637099, 1639087, +, 1989, 8680; cds-WP _002913338.1, NZ_KK737546.1, 1639149, 1639430, -, 282, 10017; cds-WP_032429680.1, NZ_KK737546.1, 1639462, 1640010, -, 549, 5430; cds-WP _002913340.1, NZ_KK737546.1, 1640010, 1640819, -, 810, 80; cds-WP _004174960.1, NZ_KK737546.1, 1640819, 1641643, -, 825, 18; cds-WP _002913342.1, NZ_KK737546.1, 1641646, 1642731, -, 1086, 36471; cds-WP_002913346.1, NZ_KK737546.1, 1642773, 1643705, -, 933, 52979; cds-WP_002913348.1, NZ_KK737546.1, 1643873, 1644424, +, 552, 1083; cds-WP _002913355.1, NZ_KK737546.1, 1644445, 1644930, -, 486, 2104; cds-WP_032429681.1, NZ_KK737546.1, 1645140, 1647284, -, 2145, 164589; cds-WP_002913358.1, NZ_KK737546.1, 1647284, 1648594, -, 1311, 70928; cds-WP _002913359.1, NZ_KK737546.1, 1648754, 1649038, -, 285, 10291; cds-WP _002913360.1, NZ_KK737546.1, 1649412, 1650734, +, 1323, 77710; cds-WP_002913362.1, NZ_KK737546.1, 1650796, 1651557, -, 762, 77524; cds-WP_004149224.1, NZ_KK737546.1, 1651847, 1652776, +, 930, 91; cds-WP _004174945.1, NZ_KK737546.1, 1653189, 1653671, +, 483, 6096; cds-WP _032428359.1, NZ_KK737546.1, 1654039, 1654920, +, 882, 11; cds-WP_021440524.1, NZ_KK737546.1, 1654930, 1655838, -, 909, 4203; cds-WP _009486224.1, NZ_KK737546.1, 1655971, 1656429, +, 459, 1; cds-WP _023282888.1, NZ_KK737546.1, 1656426, 1657622, +, 1197, 0; cds-WP_099119320.1, NZ_KK737546.1, 1657961, 1658032, +, 72, 1596; cds-WP _002913372.1, NZ_KK737546.1, 1658103, 1659317, -, 1215, 7969; cds-WP _004188912.1, NZ _KK737546.1, 1659406, 1659591, +, 186, 0; cds-WP _004153200.1, NZ_KK737546.1, 1659588, 1659701, -, 114, 3; cds-WP_004149230.1, NZ_KK737546.1, 1659700, 1661397, +,
1698, 4577; cds-WP_002913374.1, NZ_KK737546.1, 1661409, 1662146, +, 738, 10752; cds-WP_002913377.1, NZ_KK737546.1, 1662200, 1663165, -, 966, 61693; cds-WP_004154521.1, NZ_KK737546.1, 1663429, 1664664, +, 1236, 1; cds-WP _032428368.1, NZ_KK737546.1, 1664661, 1666322, -, 1662, 0; cds-WP _002913417.1, NZ_KK737546.1, 1666503, 1667495, +, 993, 75567; cds-WP_002913419.1, NZ_KK737546.1, 1667626, 1667985, +, 360, 268; cds-WP_002913421.1, NZ_KK737546.1, 1668043, 1669284, -, 1242, 537; cds-WP _002913423.1, NZ_KK737546.1, 1669631, 1670833, +, 1203, 3018; cds-WP_032429682.1, NZ_KK737546.1, 1670882, 1673053, -, 2172, 2954; cds-WP _002913434.1, NZ_KK737546.1, 1673558, 1673911, +, 354, 0; cds-WP_002913435.1, NZ_KK737546.1, 1673915, 1674307, +, 393, 36; cds-WP_004145598.1, NZ_KK737546.1, 1674359, 1675777, -, 1419, 214245; cds-WP _021440528.1, NZ_KK737546.1, 1676741, 1677667, -, 927, 322; cds-WP_021440529.1, NZ_KK737546.1, 1677756, 1678754, +, 999, 26509; cds-WP_002913440.1, NZ_KK737546.1, 1678751, 1678969, -, 219, 2152; cds-WP_004180820.1, NZ_KK737546.1, 1678971, 1680986, -, 2016, 8953; cds-WP _004180822.1, NZ_KK737546.1, 1681056, 1682117, -, 1062, 45813; cds-WP_004174922.1, NZ_KK737546.1, 1682348, 1683109, +, 762, 1262; cds-WP_002913498.1, NZ_KK737546.1, 1683286, 1684257, +, 972, 11840; cds-WP _002913505.1, NZ_KK737546.1, 1684638, 1684895, +, 258, 73258; cds-WP _021440531.1, NZ_KK737546.1, 1684940, 1686667, +, 1728, 283276; cds-WP_000522253.1, NZ_KK737546.1, 1686710, 1687219, +, 510, 63933; cds-WP_004890526.1, NZ_KK737546.1, 1687261, 1687500, -, 240, 1; cds-WP _004890524.1, NZ_KK737546.1, 1687497, 1688363, -, 867, 4612; cds-WP_004174915.1, NZ_KK737546.1, 1688446, 1689744, +, 1299, 22; cds-WP _004890521.1, NZ_KK737546.1, 1689885, 1690280, +, 396, 0; cds-WP _004890515.1, NZ_KK737546.1, 1690277, 1691188, -, 912, 317; cds-WP _002913623.1, NZ_KK737546.1, 1691307, 1692401, -, 1095, 16795; cds-WP_004890510.1, NZ_KK737546.1, 1692391, 1693266, -, 876, 4; cds-WP _002913626.1, NZ_KK737546.1, 1693266, 1694099, -, 834, 0; cds-WP _032429684.1, NZ_KK737546.1, 1694099, 1695121, -, 1023, 9; cds-AF52_RS25445, NZ_KK737546.1, 1695322, 1695665, +, 344, 12; cds-WP _020803947.1, NZ_KK737546.1, 1695761, 1696660, -, 900, 7135; cds-WP_002913630.1, NZ_KK737546.1, 1696755, 1697330, -, 576, 2569; cds-WP _002913635.1, NZ_KK737546.1, 1697392, 1697841, -, 450, 4; cds-WP_002913637.1, NZ_KK737546.1, 1697828,1698253, -, 426, 34; cds-WP_002913639.1, NZ_KK737546.1, 1698465, 1699337, +, 873, 189715; cds-WP_032429685.1, NZ_KK737546.1, 1699337, 1700236, +, 900, 3320; cds-WP_002913642.1, NZ_KK737546.1, 1700280, 1701332, -, 1053, 6; cds-WP_032428360.1, NZ_KK737546.1, 1701378, 1701863, -, 486, 0; cds-WP_004174905.1, NZ_KK737546.1, 1701875, 1702534, -, 660, 2; cds-WP_021440537.1, NZ_KK737546.1, 1702544, 1703443, -, 900, 0; cds-WP _032429686.1, NZ_KK737546.1, 1703464, 1704825, -, 1362, 28; cds-WP_021440539.1, NZ_KK737546.1, 1704837, 1706240, -, 1404, 0; cds-WP _004149260.1, NZ_KK737546.1, 1706237, 1707469, -, 1233, 0; cds-WP_032429687.1, NZ_KK737546.1, 1707669, 1708856, -, 1188, 0; cds-WP_004180842.1, NZ_KK737546.1, 1708846, 1709685, -, 840, 0; cds-WP_004152003.1, NZ_KK737546.1, 1709695, 1711098, -, 1404, 0; cds-WP_002913719.1, NZ_KK737546.1, 1711110, 1711397, -, 288, 0; cds-WP _000387720.1, NZ_KK737546.1, 1711526, 1711816, -, 291, 0; cds-WP _021440542.1, NZ_KK737546.1, 1711855, 1712871, -, 1017, 0; cds-WP _004180847.1, NZ_KK737546.1, 1712861, 1713667, -, 807, 0; cds-WP _002913725.1, NZ_KK737546.1, 1713664, 1714353, -, 690, 0; cds-WP_021440543.1, NZ_KK737546.1, 1714331, 1714810, -, 480, 0; cds-WP _002913729.1, NZ_KK737546.1, 1714823, 1715158, -, 336, 1; cds-WP _004185057.1, NZ_KK737546.1, 1715377, 1717656, -, 2280, 132791; cds-WP_002913732.1, NZ_KK737546.1, 1717951, 1718907, +, 957, 10089; cds-WP _004149269.1, NZ _KK737546.1, 1718924, 1720918, +, 1995, 1443; cds-WP _009309515.1, NZ_KK737546.1, 1720908, 1721954, -, 1047, 2; cds-WP _002913754.1, NZ_KK737546.1, 1722018, 1722614, -, 597, 107; cds-WP _032429689.1, NZ_KK737546.1, 1722673, 1724655, -, 1983, 2; cds-WP_004185072.1, NZ_KK737546.1, 1725028, 1728141, +, 3114, 251; cds-WP_100181659.1, NZ_KK737546.1, 1728269, 1728328, -, 60, 3196; cds-WP _002913764.1, NZ_KK737546.1, 1728694, 1729047, +, 354, 696; cds-WP_021440546.1, NZ_KK737546.1, 1729051, 1730178, +, 1128, 49002; cds-WP_002913768.1, NZ_KK737546.1, 1730205, 1730408, +, 204, 117194; cds-WP _002913770.1, NZ_KK737546.1, 1730457, 1731152, -, 696, 51; cds-WP _021440547.1, NZ_KK737546.1, 1731229, 1733232, -, 2004, 477; cds-WP _032429690.1, NZ_KK737546.1, 1733242, 1734117, -, 876, 5403; cds-WP _002913801.1, NZ_KK737546.1, 1734237, 1734950, -, 714, 33855; cds-WP_004149279.1, NZ_KK737546.1, 1735167, 1736201, -, 1035, 18092; cds-WP_002913803.1, NZ_KK737546.1, 1736218, 1737096, -, 879, 192853; cds-WP_002913804.1, NZ_KK737546.1, 1737250, 1737816, +, 567, 27370; cds-WP_002913805.1, NZ_KK737546.1, 1737820, 1738290, +, 471, 60443; cds-WP _002913806.1, NZ_KK737546.1, 1738352, 1739413, -, 1062, 11650; cds-WP _004221267.1, NZ_KK737546.1, 1739468, 1739584, -, 117, 6; cds-WP _002913807.1, NZ_KK737546.1, 1739636, 1741099, +, 1464, 59189; cds-WP_002913810.1, NZ_KK737546.1, 1741109, 1741468, +, 360, 89; cds-WP _023301620.1, NZ_KK737546.1, 1741596, 1742519, +, 924, 86; cds-WP_020947395.1, NZ_KK737546.1, 1742516, 1743217, -, 702, 47914; cds-WP_002913824.1, NZ_KK737546.1, 1743316, 1744602, -, 1287, 18337; cds-WP_002913827.1, NZ_KK737546.1, 1744698, 1745324, -, 627, 17491; cds-WP_021440551.1, NZ_KK737546.1, 1745542, 1746975, -, 1434, 63201; cds-WP_021440552.1, NZ_KK737546.1, 1746985, 1747878, -, 894, 1032; cds-WP _004149288.1, NZ_KK737546.1, 1748142, 1749179, +, 1038, 883; cds-WP _004145656.1, NZ_KK737546.1, 1749176, 1749817, +, 642, 20; cds-WP_002913838.1, NZ_KK737546.1, 1749998, 1752058, +, 2061, 134090; cds-WP _002913839.1, NZ_KK737546.1, 1752062, 1753594, +, 1533, 98283; cds-WP _032429691.1, NZ_KK737546.1, 1753648, 1755876, -, 2229, 15088; cds-WP_004174861.1, NZ_KK737546.1, 1756247, 1756420, +, 174, 58; cds-WP_004221278.1, NZ_KK737546.1, 1756517, 1757428, -, 912, 8; cds-WP _021440556.1, NZ_KK737546.1, 1757502, 1758734, +, 1233, 0; cds-WP _021440557.1, NZ_KK737546.1, 1759028, 1760206, +, 1179, 0; cds-WP_009309501.1, NZ_KK737546.1, 1760190, 1762058, +, 1869, 3; cds-WP_004151979.1, NZ_KK737546.1, 1762153, 1763730, -, 1578, 64132; cds-WP _004151980.1, NZ_KK737546.1, 1763798, 1765264, -, 1467, 9536; cds-WP _015958798.1, NZ_KK737546.1, 1765423, 1766814, +, 1392, 21156; cds-WP _004144303.1, NZ_KK737546.1, 1766798, 1767016, -, 219, 74; cds-WP _021313079.1, NZ_KK737546.1, 1767066, 1768145, -, 1080, 17; cds-WP_020317242.1, NZ_KK737546.1, 1768329, 1768808, -, 480, 0; cds-WP_032418518.1, NZ_KK737546.1, 1769030, 1769722, -, 693, 2; cds-WP_004144309.1, NZ_KK737546.1, 1770089, 1771567, -, 1479,6638; cds-WP_002913889.1, NZ_KK737546.1, 1771681,1772859, -, 1179, 144090; cds-WP_002913890.1, NZ_KK737546.1, 1772870, 1773490, -, 621, 83909; cds-WP_004149335.1, NZ_KK737546.1, 1773525, 1774799, -, 1275, 62983; cds-WP _002913892.1, NZ_KK737546.1, 1774891, 1776012, -, 1122, 12687; cds-WP_004149336.1, NZ_KK737546.1, 1776039, 1777034, -, 996, 14143; cds-WP_002913894.1, NZ_KK737546.1, 1777311, 1778477, -, 1167, 7248; cds-WP_004144312.1, NZ_KK737546.1, 1778961, 1779392, -, 432, 214962; cds-WP_004890414.1, NZ_KK737546.1, 1779553, 1781877, -, 2325, 31; cds-WP_021440561.1, NZ_KK737546.1, 1781878, 1786827, -, 4950, 132278; cds-WP_002913950.1, NZ_KK737546.1, 1787033, 1787890, +, 858, 26269; cds-WP _032429692.1, NZ_KK737546.1, 1788117, 1788959, +, 843, 19; cds-WP_021440563.1, NZ_KK737546.1, 1789030, 1789806, -, 777, 12820; cds-WP_002913953.1, NZ_KK737546.1, 1789905, 1791191, -, 1287, 61183; cds-WP _002913954.1, NZ_KK737546.1, 1791262, 1791462, -, 201, 55548; cds-WP _002913956.1, NZ_KK737546.1, 1791464, 1791799, -, 336, 45297; cds-WP_032429693.1, NZ_KK737546.1, 1791801, 1793651, -, 1851, 93323; cds-WP_004174835.1, NZ_KK737546.1, 1793667, 1794182, -, 516, 48685; cds-WP _002913979.1, NZ_KK737546.1, 1794257, 1794580, -, 324, 15296; cds-WP _002913991.1, NZ_KK737546.1, 1794598, 1794984, -, 387, 3136; cds-WP_002913992.1, NZ_KK737546.1, 1795011, 1796225, -, 1215, 73703; cds-WP_002913993.1, NZ_KK737546.1, 1796404, 1796895, -, 492, 46; cds-WP _004185116.1, NZ_KK737546.1, 1797130, 1797864, -, 735, 5547; cds-WP_002913995.1, NZ_KK737546.1, 1797985, 1798788, +, 804, 17345; cds-WP_021440564.1, NZ_KK737546.1, 1798835, 1799671, -, 837, 0; cds-WP_021440565.1, NZ_KK737546.1, 1799771, 1800751, -, 981, 0; cds-WP _023301580.1, NZ_KK737546.1, 1800742, 1801380, -, 639, 9638; cds-WP_002914002.1, NZ_KK737546.1, 1801505, 1802782, +, 1278, 1407; cds-WP_004144329.1, NZ_KK737546.1, 1802779, 1803915, -, 1137, 442; cds-WP _021440567.1, NZ_KK737546.1, 1803998, 1804831, -, 834, 0; cds-WP _002914018.1, NZ_KK737546.1, 1804831, 1806267, -, 1437, 3; cds-WP _015958809.1, NZ_KK737546.1, 1806337, 1809612, -, 3276, 5; cds-WP_004214134.1, NZ_KK737546.1, 1809725, 1810921, +, 1197, 1378; cds-WP_002914024.1, NZ_KK737546.1, 1810995, 1811417, +, 423, 1035; cds-WP_002914027.1, NZ_KK737546.1, 1811473, 1812726, -, 1254, 504254; cds-WP _004185137.1, NZ_KK737546.1, 1813052, 1814242, +, 1191, 7423; cds-WP _002914032.1, NZ_KK737546.1, 1814317, 1814655, -, 339, 448; cds-WP_004149357.1, NZ_KK737546.1, 1814721, 1816058, -, 1338, 7260; cds-WP _002914033.1, NZ_KK737546.1, 1816045, 1816731, -, 687, 9857; cds-WP _004888415.1, NZ_KK737546.1, 1816761, 1818182, -, 1422, 3293; cds-WP _004185139.1, NZ_KK737546.1, 1818773, 1822660, -, 3888, 23278; cds-WP_004144346.1, NZ_KK737546.1, 1822836, 1824452, +, 1617, 4497; cds-WP_071603195.1, NZ_KK737546.1, 1824449, 1824991, -, 543, 10; cds-WP _004180922.1, NZ _KK737546.1, 1825021, 1825656, -, 636, 1; cds-WP_004144349.1, NZ _KK737546.1, 1825870, 1826718, +, 849, 281; cds-WP _004888420.1, NZ _KK737546.1, 1826775, 1827035, +, 261, 4442; cds-WP_004144351.1, NZ_KK737546.1, 1827048, 1827428, -, 381, 26874; cds-WP _004888422.1, NZ_KK737546.1, 1827428, 1828159, -, 732, 28083; cds-WP_002914062.1, NZ_KK737546.1, 1828171, 1828908, -, 738, 18679; cds-WP _002914063.1, NZ_KK737546.1, 1828920, 1829825, -, 906, 44735; cds-WP _002914065.1, NZ_KK737546.1, 1829822, 1830502, -, 681, 5145; cds-WP_002914067.1, NZ_KK737546.1, 1830752, 1831726, -, 975, 90827; cds-WP _002914069.1, NZ_KK737546.1, 1831742, 1833541, -, 1800, 91992; cds-WP_004144354.1, NZ _KK737546.1, 1833727, 1834206, -, 480, 12007; cds-WP _002914070.1, NZ_KK737546.1, 1834203, 1835159, -, 957, 47371; cds-WP_002914072.1, NZ_KK737546.1, 1835159, 1835809, -, 651, 172503; cds-WP _002914074.1, NZ_KK737546.1, 1835842, 1836417, -, 576, 148341; cds-WP_121980491.1, NZ _KK737546.1, 1836414, 1836509, -, 96, 43; cds-WP _004144357.1, NZ _KK737546.1, 1836838, 1838457, +, 1620, 720; cds-WP_002914079.1, NZ_KK737546.1, 1838442, 1839179, -, 738, 5; cds-WP_002914082.1, NZ_KK737546.1, 1839311, 1840642, +, 1332, 1965; cds-WP_002914084.1, NZ_KK737546.1, 1840688, 1841071, -, 384, 1; cds-WP _002914088.1, NZ_KK737546.1, 1841384, 1842073, +, 690, 1098; cds-WP _002914089.1, NZ_KK737546.1, 1842131, 1843216, -, 1086, 218; cds-WP_002914091.1, NZ_KK737546.1, 1843420, 1843845, +, 426, 266; cds-WP _002914092.1, NZ_KK737546.1, 1843915, 1844613, +, 699, 23528; cds-WP _004149373.1, NZ_KK737546.1, 1844648, 1847299, +, 2652, 147388; cds-WP_002914094.1, NZ_KK737546.1, 1847420, 1848775, +, 1356, 39916; cds-WP _004888435.1, NZ_KK737546.1, 1848817, 1849140, +, 324, 1023; cds-WP _004144368.1, NZ_KK737546.1, 1849144, 1850442, -, 1299, 34790; cds-AF52_RS14425, NZ_KK737547.1, 98, 328, -, 231, 0; cds-WP_002890376.1, NZ_KK737547.1, 349, 636, -, 288, 0; cds-AF52_RS0123765, NZ_KK737547.1, 642, 842, -, 201, 25; cds-AF52_RS27035, NZ_KK737547.1, 876, 1049, +, 174, 232; cds-AF52_RS14415, NZ_KK737547.1, 4070, 4221, +, 152, 209; cds-AF52_RS14410, NZ_KK737547.1, 4277, 4528, -, 252, 14; cds-WP _002890386.1, NZ_KK737547.1, 4875, 5477, -, 603, 47035; cds-AF52_RS14400, NZ_KK737547.1, 5700, 5873, -, 174, 318; cds-AF52_RS27040, NZ_KK737547.1, 7013, 7174, -, 162, 291; cds-WP _032429763.1, NZ_KK737547.1, 7192, 7758, -, 567, 0; cds-WP _032429764.1, NZ_KK737547.1, 7833, 7949, +, 117, 0; cds-WP _002890390.1, NZ_KK737547.1, 8014, 9078, +, 1065, 9958; cds-WP_002890395.1, NZ_KK737547.1, 9187, 10314, +, 1128, 211181; cds-WP_002890398.1, NZ_KK737547.1, 10337, 10669, +, 333, 74802; cds-WP_002890400.1, NZ_KK737547.1, 10696, 12543, +, 1848, 122457; cds-WP_002890403.1, NZ_KK737547.1,
12554, 13525, +, 972, 23908; cds-WP _002890404.1, NZ_KK737547.1, 13662, 14027, +, 366, 0; cds-WP _004144646.1, NZ_KK737547.1, 14052, 14747, +, 696, 2; cds-WP_002890412.1, NZ_KK737547.1, 14802, 15686, -, 885, 234877; cds-WP _002890414.1, NZ _KK737547.1, 15985, 16524, -, 540, 4; cds-WP _002890417.1, NZ_KK737547.1, 16672, 17121, +, 450, 129; cds-WP _004144650.1, NZ_KK737547.1, 17125, 18228, +, 1104, 2089; cds-WP_001021161.1, NZ_KK737547.1, 18316, 18786, +, 471, 18355; cds-WP_002891356.1, NZ_KK737547.1, 18806, 19225, +, 420, 57928; cds-WP_004177267.1, NZ_KK737547.1, 19297, 20268, +, 972, 29559; cds-WP_004177266.1, NZ_KK737547.1, 20261, 20764, +, 504, 177; cds-WP _004191727.1, NZ_KK737547.1, 20810, 21784, -, 975, 2; cds-WP_004144655.1, NZ_KK737547.1, 21845, 23707, -, 1863, 1926; cds-WP_004144656.1, NZ_KK737547.1, 23726, 24625, -, 900, 31216; cds-WP _032429766.1, NZ_KK737547.1, 24626, 24868, -, 243, 10082; cds-WP _004151340.1, NZ_KK737547.1, 25050, 26498, +, 1449, 2250; cds-WP_032429768.1, NZ_KK737547.1, 26565, 27041, -, 477, 855; cds-WP_002891460.1, NZ_KK737547.1, 27092, 27685, -, 594, 0; cds-WP_002891462.1, NZ_KK737547.1, 27648, 28559, -, 912, 3737; cds-WP_002891465.1, NZ_KK737547.1, 28751, 29242, +, 492, 40395; cds-WP _004144660.1, NZ_KK737547.1, 29286, 30650, -, 1365, 1650; cds-WP_004177261.1, NZ_KK737547.1, 30810, 31988, -, 1179, 5116; cds-WP _002891473.1, NZ_KK737547.1, 32320, 33684, -, 1365, 3; cds-WP_002891476.1, NZ_KK737547.1, 33695, 34513, -, 819, 0; cds-WP_032103307.1, NZ_KK737547.1, 34588, 36267, -, 1680, 0; cds-WP _002891541.1, NZ_KK737547.1, 36278, 36598, -, 321, 0; cds-WP_032429771.1, NZ_KK737547.1, 36612, 38288, -, 1677, 19192; cds-WP _032419345.1, NZ_KK737547.1, 38285, 39106, -, 822, 0; cds-WP _002891624.1, NZ_KK737547.1, 39259, 40017, +, 759,158; cds-WP_004178748.1, NZ_KK737547.1, 40057, 40389, -, 333, 8; cds-WP_002891629.1, NZ_KK737547.1, 40639, 41526, -, 888, 300094; cds-WP_002891634.1, NZ_KK737547.1, 41538, 41867, -, 330, 236106; cds-WP_002891635.1, NZ_KK737547.1, 41867, 42478, -, 612, 347651; cds-WP _032424651.1, NZ_KK737547.1, 42468, 44459, -, 1992, 1820420; cds-WP_002891641.1, NZ_KK737547.1, 44479, 45423, -, 945, 987323; cds-WP_032429776.1, NZ_KK737547.1, 45903, 47378, -, 1476, 13639; cds-WP_002891647.1, NZ_KK737547.1, 47422, 48000, -, 579, 8225; cds-WP _032429779.1, NZ_KK737547.1, 48303, 48620, +, 318, 3804; cds-WP _004147333.1, NZ_KK737547.1, 48966, 50264, +, 1299, 387426; cds-WP _002891804.1, NZ_KK737547.1, 50574, 51197, +, 624, 17980; cds-WP_002891807.1, NZ_KK737547.1, 51448, 52722, +, 1275, 95680; cds-WP _004151336.1, NZ_KK737547.1, 52906, 55260, +, 2355, 220166; cds-WP_002444653.1, NZ_KK737547.1, 55470, 55742, +, 273, 53188; cds-WP _004177252.1, NZ_KK737547.1, 55957, 57831, +, 1875, 231193; cds-WP _004144680.1, NZ_KK737547.1, 57980, 58348, +, 369, 0; cds-WP _002891860.1, NZ_KK737547.1, 58460, 58861, +, 402, 2473; cds-WP_002891863.1, NZ_KK737547.1, 58973, 59674, -, 702, 27590; cds-WP_032429782.1, NZ_KK737547.1, 59741, 61441, -, 1701, 207; cds-WP _004151335.1, NZ_KK737547.1, 61569, 62387, +, 819, 5; cds-WP _002891871.1, NZ_KK737547.1, 62432, 63484, -, 1053, 2885; cds-WP_002891873.1, NZ_KK737547.1, 63600, 64058, +, 459, 5239; cds-WP_032429784.1, NZ_KK737547.1, 64102, 65874, +, 1773, 71; cds-WP _032429785.1, NZ_KK737547.1, 65867, 67645, +, 1779, 112; cds-WP_002891893.1, NZ_KK737547.1, 67889, 68227, +, 339, 0; cds-WP _004152876.1, NZ_KK737547.1, 68261, 69547, +, 1287, 833; cds-WP_020804789.1, NZ_KK737547.1, 69602, 70465, -, 864, 7203; cds-WP_002891903.1, NZ_KK737547.1, 70682, 71173, +, 492, 38; cds-WP _004142687.1, NZ_KK737547.1, 71211, 71522, -, 312, 3963; cds-WP _004178755.1, NZ_KK737547.1, 71590, 71997, -, 408, 165; cds-WP_013815099.1-31, NZ_KK737547.1, 72608, 73576, -, 969, 6354; cds-WP _004147373.1, NZ_KK737547.1, 74028, 77174, -, 3147, 345479; cds-WP_004177236.1, NZ_KK737547.1, 77197, 78390, -, 1194, 402111; cds-WP_002892080.1, NZ_KK737547.1, 78533, 79183, +, 651, 10375; cds-WP _032429786.1, NZ_KK737547.1, 79313, 82660, +, 3348, 128850; cds-WP _032429788.1, NZ_KK737547.1, 82696, 83595, -, 900, 33; cds-WP_002892136.1, NZ_KK737547.1, 83663, 83836, -, 174, 4; cds-WP_002892142.1, NZ_KK737547.1, 83849, 84376, -, 528, 0; cds-WP _002892144.1, NZ_KK737547.1, 84446, 84823, +, 378, 12; cds-WP _002892145.1, NZ_KK737547.1, 84974, 85525, +, 552, 638; cds-WP _032429789.1, NZ_KK737547.1, 85618, 87525, +, 1908, 6661; cds-WP_002892173.1, NZ_KK737547.1, 87583, 87915, +, 333, 1252; cds-WP_002892177.1, NZ_KK737547.1, 87915, 88520, +, 606, 344; cds-WP_002892181.1, NZ_KK737547.1, 88632, 90506, +, 1875, 54764; cds-WP_032423667.1, NZ_KK737547.1, 90729, 91373, +, 645, 96701; cds-WP _002892189.1, NZ_KK737547.1, 91502, 92464, +, 963, 46356; cds-WP_002892192.1, NZ_KK737547.1, 92528, 93832, +, 1305, 6766; cds-WP_002892195.1, NZ_KK737547.1, 93868, 95544, -, 1677, 7495; cds-WP_004147385.1, NZ_KK737547.1, 95770, 96990, -, 1221, 1; cds-WP _002892203.1, NZ_KK737547.1, 97260, 98912, +, 1653, 6957; cds-WP _002892205.1, NZ_KK737547.1, 99018, 99497, -, 480, 172; cds-WP_004151327.1, NZ_KK737547.1, 99700, 100494, -, 795, 8944; cds-WP_004893800.1, NZ_KK737547.1, 100626, 103127, -, 2502, 7294; cds-WP_002892208.1, NZ_KK737547.1, 103234, 103644, +, 411, 985; cds-WP _004893797.1, NZ_KK737547.1, 103641, 104099, -, 459, 0; cds-WP _002892258.1, NZ_KK737547.1, 104096, 105013, -, 918, 2274; cds-WP _004893793.1, NZ_KK737547.1, 105154, 105831, +, 678, 3232; cds-WP_004893788.1, NZ_KK737547.1, 105818, 106600, +, 783, 5594; cds-WP_002892263.1, NZ_KK737547.1, 106708, 107562, -, 855, 88484; cds-WP_009484702.1, NZ_KK737547.1, 107621, 108430, -, 810, 42; cds-WP _004196998.1, NZ_KK737547.1, 108420, 109043, -, 624, 4; cds-WP _032429794.1, NZ_KK737547.1, 109014, 109700, +, 687, 0; cds-WP_032429796.1, NZ_KK737547.1, 109697, 112111, +, 2415, 21631; cds-WP_004219504.1, NZ_KK737547.1, 112088, 112201, -, 114, 0; cds-WP _004142997.1, NZ_KK737547.1, 112293, 113438, +, 1146, 2; cds-WP _004151323.1, NZ_KK737547.1, 113546, 114622, -, 1077, 1; cds-WP_032429798.1, NZ_KK737547.1, 114734, 115801, -, 1068, 5352; cds-WP_004151322.1, NZ_KK737547.1, 115798, 116307, -, 510, 2357; cds-WP _032429801.1, NZ_KK737547.1, 116404, 116742, -, 339, 0; cds-WP _004151320.1, NZ_KK737547.1, 116850, 117572, -, 723, 369; cds-WP_004151319.1, NZ_KK737547.1, 117576, 118070, -, 495, 13418; cds-WP _004143010.1, NZ_KK737547.1, 118246, 119631, +, 1386, 18722; cds-WP_004143016.1, NZ_KK737547.1, 119677, 119889, -, 213, 0; cds-WP_004143017.1, NZ_KK737547.1, 119891, 120757, -, 867, 8315; cds-WP_013815099.1-32, NZ_KK737547.1, 121337, 122305, +, 969, 4154; cds-AF52_RS25480, NZ_KK737547.1, 122362, 122511, +, 150, 58; cds-WP _002893005.1, NZ_KK737547.1, 122529, 123590, +, 1062, 0; cds-AF52_RS13845, NZ_KK737547.1, 123655, 124524, +, 870, 0; cds-WP_013815099.1-33, NZ_KK737547.1, 124555, 125523, +, 969, 6426; cds-AF52_RS27075, NZ_KK737547.1, 125582, 125707, +, 126, 57; cds-AF52_RS13835, NZ_KK737547.1, 125760, 126170, -, 411, 0; cds-AF52_RS27080, NZ_KK737547.1, 127801, 127981, -, 181, 266; cds-AF52_RS13830, NZ_KK737547.1, 128015, 130339, -, 2325, 92; cds-WP _021441375.1, NZ_KK737547.1, 130436, 131470, -, 1035, 86; cds-WP_004151823.1, NZ_KK737547.1, 132136, 133002, -, 867, 47; cds-WP_004223482.1, NZ_KK737547.1, 133111, 134538, +, 1428, 0; cds-WP _004142570.1, NZ_KK737547.1, 134609, 135853, +, 1245, 6623; cds-WP _004176981.1, NZ_KK737547.1, 135857, 136837, -, 981, 1932; cds-WP _004183289.1, NZ_KK737547.1, 137086, 137502, +, 417, 0; cds-WP_016946855.1, NZ_KK737547.1, 137522, 138673, -, 1152, 15335; cds-WP _032429809.1, NZ_KK737547.1, 138675, 139697, -, 1023, 2; cds-WP _021441372.1, NZ_KK737547.1, 139714, 140919, -, 1206, 2; cds-WP _021441371.1, NZ_KK737547.1, 141107, 142483, -, 1377, 1; cds-WP_032428635.1, NZ_KK737547.1, 142591, 144789, -, 2199, 0; cds-WP_032428633.1, NZ_KK737547.1, 144849, 145931, -, 1083, 0; cds-WP _032428631.1, NZ_KK737547.1, 146327, 147811, -, 1485, 0; cds-WP _004176990.1, NZ_KK737547.1, 147970, 149271, +, 1302, 22; cds-WP _002892824.1, NZ_KK737547.1, 149285, 149650, +, 366, 0; cds-WP_021441367.1, NZ_KK737547.1, 150030, 151529, +, 1500, 0; cds-WP_021441366.1, NZ_KK737547.1, 151507, 152049, +, 543, 15081; cds-WP _004178885.1, NZ_KK737547.1, 152067, 152429, -, 363, 39328; cds-WP _004178884.1, NZ_KK737547.1, 152430, 152732, -, 303, 27508; cds-WP _021441365.1, NZ _KK737547.1, 152816, 153931, -, 1116, 26574; cds-WP _004183273.1, NZ_KK737547.1, 153948, 155120, -, 1173, 2676; cds-WP _002892774.1, NZ_KK737547.1, 155238, 155921, -, 684, 13926; cds-WP_009308086.1, NZ_KK737547.1, 156346, 157533, +, 1188, 7; cds-WP_004223434.1, NZ_KK737547.1, 157578, 159311, +, 1734, 0; cds-WP _004183270.1, NZ_KK737547.1, 159399, 160175, +, 777, 0; cds-WP _032429971.1, NZ_KK737547.1, 160201, 160935, +, 735, 172; cds-WP _013815099.1-34, NZ_KK737547.1, 160961, 161929, -, 969, 4801; cds-AF52_RS13690, NZ_KK737547.1, 161961, 162431, -, 471, 0; cds-WP_021441356.1, NZ_KK737547.1, 162670, 163593, +, 924, 0; cds-WP _004147437.1, NZ_KK737547.1, 163758, 165275, +, 1518,0; cds-WP_021441355.1, NZ_KK737547.1, 165380, 166750, +, 1371,0; cds-WP_021441354.1, NZ_KK737547.1, 166750, 168150, +, 1401, 0; cds-WP_187079191.1, NZ_KK737547.1, 168174, 169184, +, 1011, 0; cds-WP_004177009.1, NZ_KK737547.1, 169200, 169841, -, 642, 0; cds-WP _004152946.1, NZ_KK737547.1, 170025, 171047, -, 1023, 0; cds-WP_004142660.1, NZ_KK737547.1, 171067, 172551, -, 1485, 0; cds-WP_002892597.1, NZ_KK737547.1, 172584, 173507, -, 924, 0; cds-WP_004191476.1, NZ_KK737547.1, 173937, 174956, -, 1020, 0; cds-AF52_RS13635, NZ_KK737547.1, 175058, 176062, +, 1005, 0; cds-WP_004147428.1, NZ_KK737547.1, 176984, 178015, -, 1032, 0; cds-WP_004191478.1, NZ_KK737547.1, 178025, 179197, -, 1173, 0; cds-WP _004215104.1, NZ_KK737547.1, 179194, 180114, -, 921, 0; cds-WP_187079192.1, NZ_KK737547.1, 180220, 181188, -, 969, 3812; cds-AF52_RS0123810, NZ_KK737547.1, 181222, 181695, +, 474, 641; cds-WP _032429814.1, NZ_KK737547.1, 181760, 183334, -, 1575, 208; cds-WP _002892402.1, NZ_KK737547.1, 183628, 183900, -, 273, 11736; cds-WP _002892400.1, NZ_KK737547.1, 184001, 184921, -, 921, 131199; cds-WP _032429816.1, NZ_KK737547.1, 185432, 186298, +, 867, 2387; cds-WP _032428620.1, NZ_KK737547.1, 186371, 187675, -, 1305, 0; cds-WP_004142684.1, NZ_KK737547.1, 187721, 187861, -, 141, 0; cds-WP _002892370.1, NZ_KK737547.1, 188186, 188356, +, 171, 0; cds-AF52_RS27950, NZ_KK737547.1, 188435, 188512, -, 78, 0; cds-WP _013815099.1-35, NZ _KK737547.1, 188546, 189514, +, 969, 8529; cds-AF52_RS13575, NZ_KK737547.1, 189571, 190284, -, 714, 1; cds-WP_004183295.1, NZ_KK737547.1, 190287, 191030, -, 744, 0; cds-WP_004176974.1, NZ_KK737547.1, 191045, 191515, -, 471, 0; cds-WP _002893017.1, NZ_KK737547.1, 191508, 191942, -, 435, 0; cds-WP _004178896.1, NZ_KK737547.1, 192190, 192843, -, 654, 136; cds-WP _004142557.1, NZ_KK737547.1, 192963, 193331, -, 369, 3837; cds-WP _014599061.1, NZ_KK737547.1, 193331, 193912, -, 582, 741; cds-WP_171991789.1, NZ_KK737547.1, 194092, 195207, +, 1116, 13; cds-WP_002893035.1, NZ_KK737547.1, 195238, 195579, +, 342, 340; cds-WP_002893037.1, NZ_KK737547.1, 195597, 195845, -, 249, 261; cds-WP_032428645.1, NZ_KK737547.1, 196348, 199071, +, 2724, 7; cds-WP_021441380.1, NZ_KK737547.1, 199124, 200203, +, 1080, 0; cds-WP_032429823.1, NZ_KK737547.1, 200217, 203309, +, 3093, 0; cds-WP _004176956.1, NZ_KK737547.1, 203658, 204377, -, 720, 5966; cds-WP _004178906.1, NZ_KK737547.1, 204425, 205540, -, 1116, 3; cds-WP _021441381.1, NZ_KK737547.1, 205867, 207534, +, 1668, 0; cds-WP _004178910.1, NZ_KK737547.1, 207553, 208506, +, 954, 0; cds-WP_008805546.1, NZ_KK737547.1, 208509, 209402, +, 894, 0; cds-WP _004223507.1, NZ_KK737547.1, 209406, 210443, +, 1038, 0; cds-WP_021441382.1, NZ_KK737547.1, 210433, 211443, +, 1011, 4; cds-WP _021441383.1, NZ_KK737547.1, 211541, 212809, +, 1269, 0; cds-WP _032428650.1, NZ_KK737547.1, 212913, 213821, -, 909, 0; cds-WP_032429824.1, NZ_KK737547.1, 213946, 215280, +, 1335, 0; cds-WP _004176948.1, NZ_KK737547.1, 215277, 216740, -, 1464, 0; cds-WP_004176946.1, NZ_KK737547.1, 216982, 217833, +, 852, 0; cds-WP _002893187.1, NZ_KK737547.1, 217881, 218522, +, 642, 6; cds-WP _002893189.1, NZ_KK737547.1, 218537, 219202, +, 666, 0; cds-WP_004151676.1, NZ_KK737547.1, 219195, 219956, +, 762, 0; cds-WP _032428652.1, NZ_KK737547.1, 220010, 220774, -, 765, 0; cds-WP _004183313.1, NZ_KK737547.1, 220955, 222292, +, 1338, 0; cds-WP_104443363.1, NZ_KK737547.1, 222667, 222822, -, 156, 0; cds-WP_077254746.1, NZ_KK737547.1, 223649, 223759, -, 111, 22; cds-WP_013815099.1-
36, NZ_KK737547.1, 223803, 224771, -, 969, 6537; cds-WP _022615459.1, NZ_KK737547.1, 225071, 226066, -, 996, 2917; cds-WP _009483809.1, NZ_KK737547.1, 226423, 227127, -, 705, 119; cds-WP_021441389.1, NZ_KK737547.1, 227114, 227950, -, 837, 3; cds-WP_032429827.1, NZ_KK737547.1, 227943, 228776, -, 834, 117; cds-WP _021441390.1, NZ_KK737547.1, 228776, 229798, -, 1023, 13; cds-WP_009308119.1, NZ_KK737547.1, 229918, 231486, -, 1569, 1509; cds-WP _002893466.1, NZ_KK737547.1, 231692, 232387, +, 696, 9; cds-WP _032429829.1, NZ_KK737547.1, 232508, 234172, -, 1665, 31397; cds-WP _004147503.1, NZ_KK737547.1, 234186, 235658, -, 1473, 8505; cds-WP _004151671.1, NZ_KK737547.1, 235672, 236259, -, 588, 13; cds-WP_004142478.1, NZ_KK737547.1, 236388, 238421, +, 2034, 31; cds-WP_071603220.1, NZ_KK737547.1, 238467, 238994, -, 528, 132; cds-WP_021441392.1, NZ_KK737547.1, 239138, 240394, +, 1257, 13; cds-WP _021441393.1, NZ_KK737547.1, 240391, 241284, -, 894, 1; cds-WP _004183326.1, NZ_KK737547.1, 241539, 242144, +, 606, 788; cds-WP_021441395.1, NZ_KK737547.1, 242203, 243702, -, 1500, 476511; cds-WP_032428654.1, NZ_KK737547.1, 243717, 245135, -, 1419, 846558; cds-WP _004893550.1, NZ_KK737547.1, 245170, 246120, -, 951, 363393; cds-WP _004893549.1, NZ_KK737547.1, 246113, 246943, -, 831, 589397; cds-WP_032429976.1, NZ_KK737547.1, 247255, 248205, +, 951, 1003; cds-WP_021441398.1, NZ_KK737547.1, 248202, 249164, -, 963, 15051; cds-WP _004885708.1, NZ_KK737547.1, 249320, 250408, -, 1089, 360871; cds-WP_032429833.1, NZ_KK737547.1, 250410, 251936, -, 1527, 1320731; cds-WP_032428655.1, NZ_KK737547.1, 251936, 252544, -, 609, 600641; cds-WP _021441401.1, NZ_KK737547.1, 252555, 253496, -, 942, 396104; cds-WP_021441402.1, NZ_KK737547.1, 253614, 254273, -, 660, 14316; cds-WP _021441403.1, NZ_KK737547.1, 254340, 256568, -, 2229, 48187; cds-WP_004147519.1, NZ_KK737547.1, 256828, 258036, +, 1209, 548; cds-WP_004176919.1, NZ_KK737547.1, 258064, 258267, +, 204, 145; cds-WP_021441404.1, NZ_KK737547.1, 258278, 262159, +, 3882, 1762; cds-WP_002893737.1, NZ_KK737547.1, 262224, 263018, -, 795, 17; cds-WP _004147521.1, NZ_KK737547.1, 263015, 264007, -, 993, 2; cds-WP _032429835.1, NZ_KK737547.1, 264004, 265011, -, 1008, 14; cds-WP_032429836.1, NZ_KK737547.1, 265124, 266365, +, 1242, 0; cds-WP _004147526.1, NZ_KK737547.1, 266628, 267587, -, 960, 35; cds-WP_032429837.1, NZ_KK737547.1, 267776, 268951, +, 1176, 2973; cds-WP _004147529.1, NZ_KK737547.1, 268961, 270568, +, 1608, 1667; cds-WP_020324582.1, NZ_KK737547.1, 270582, 271433, +, 852, 16; cds-WP _024622880.1, NZ_KK737547.1, 271442, 272188, +, 747, 3; cds-WP_002893892.1, NZ_KK737547.1, 272189, 272602, +, 414, 1; cds-WP_021441407.1, NZ_KK737547.1, 272959, 275064, +, 2106, 69280; cds-WP _002893903.1, NZ_KK737547.1, 275169, 275366, +, 198, 32419; cds-WP _004178982.1, NZ_KK737547.1, 275363, 275767, -, 405, 32; cds-WP _021441408.1, NZ_KK737547.1, 275742, 276044, -, 303, 59; cds-WP_021464368.1, NZ_KK737547.1, 276228, 276971, -, 744, 5; cds-WP _032429838.1, NZ_KK737547.1, 277029, 278117, -, 1089, 0; cds-WP _020801584.1, NZ_KK737547.1, 278281, 279783, +, 1503, 0; cds-WP _002894035.1, NZ_KK737547.1, 279780, 280778, +, 999, 0; cds-WP_002894036.1, NZ_KK737547.1, 280799, 281863, +, 1065, 1; cds-WP _004178989.1, NZ_KK737547.1, 281965, 283206, +, 1242, 5699; cds-WP_004210859.1, NZ_KK737547.1, 283420, 284619, -, 1200, 9; cds-WP _004183373.1, NZ_KK737547.1, 284721, 285749, +, 1029, 0; cds-WP_032429839.1, NZ_KK737547.1, 285810, 287078, -, 1269, 0; cds-WP _032429841.1, NZ_KK737547.1, 287104, 288462, -, 1359, 13; cds-WP_004151655.1, NZ_KK737547.1, 288573, 289382, -, 810, 1; cds-WP_072157843.1, NZ_KK737547.1, 289504, 290454, -, 951,108; cds-WP_032429842.1, NZ_KK737547.1, 290600, 291838, -, 1239, 0; cds-WP _002894159.1, NZ_KK737547.1, 291835, 293070, -, 1236, 1; cds-WP_032429843.1, NZ_KK737547.1, 293067, 294329, -, 1263, 0; cds-WP_002894164.1, NZ_KK737547.1, 294781, 295389, +, 609, 1474; cds-WP _032429844.1, NZ_KK737547.1, 295402, 296898, +, 1497, 0; cds-WP_032429847.1, NZ_KK737547.1, 296895, 297923, +, 1029, 1; cds-WP_032429849.1, NZ_KK737547.1, 297966, 298907, -, 942, 1; cds-WP_004223619.1, NZ_KK737547.1, 298919, 299923, -, 1005, 0; cds-WP_004147555.1, NZ_KK737547.1, 299920, 300909, -, 990, 0; cds-WP_021441413.1, NZ_KK737547.1, 300906, 302408, -, 1503, 0; cds-WP_032429851.1, NZ_KK737547.1, 302453, 303433, -, 981, 0; cds-WP_032429853.1, NZ_KK737547.1, 303842, 306151, +, 2310, 0; cds-WP_002894357.1, NZ_KK737547.1, 306259, 306801, -, 543, 0; cds-WP _004142397.1, NZ_KK737547.1, 306798, 307487, -, 690, 47; cds-WP_020802207.1, NZ_KK737547.1, 307629, 308774, +, 1146, 1; cds-WP_004212613.1, NZ_KK737547.1, 308775, 309401, -, 627, 4; cds-WP_002894369.1, NZ_KK737547.1, 309386, 310609, -, 1224, 31; cds-WP _004147565.1, NZ_KK737547.1, 310720, 311652, -, 933, 0; cds-WP_002894375.1, NZ_KK737547.1, 311833, 312582, -, 750, 251; cds-WP_002894394.1, NZ_KK737547.1, 312994, 313557, +, 564, 125584; cds-WP_004183398.1, NZ_KK737547.1, 313745, 315310, +, 1566, 7867; cds-WP_002894401.1, NZ_KK737547.1, 315387, 315815, -, 429, 1147; cds-WP_002894404.1, NZ_KK737547.1, 316002, 316412, -, 411, 135; cds-WP_032429854.1, NZ_KK737547.1, 316591, 317388, -, 798, 11; cds-WP _004142371.1, NZ_KK737547.1, 317481, 318854, -, 1374, 7007; cds-WP_072143258.1, NZ_KK737547.1, 319136, 319711, +, 576, 10535; cds-WP_002439184.1, NZ_KK737547.1, 319906, 320115, +, 210, 73246; cds-WP_002894459.1, NZ_KK737547.1, 320183, 320566, -, 384, 166; cds-WP _002894461.1, NZ_KK737547.1, 320656, 321444, +, 789, 22; cds-WP_002894464.1, NZ_KK737547.1, 321687, 321893, +, 207, 2267; cds-WP_002894467.1, NZ_KK737547.1, 322084, 323049, -, 966, 45573; cds-WP_020803604.1, NZ_KK737547.1, 323230, 324189, -, 960, 81; cds-WP_002894472.1, NZ_KK737547.1, 324320, 324964, -, 645, 2502; cds-WP _002894474.1, NZ_KK737547.1, 325069, 325332, -, 264, 652; cds-WP _002894539.1, NZ_KK737547.1, 325445, 326644, -, 1200, 38637; cds-WP _004147579.1, NZ_KK737547.1, 326783, 327931, -, 1149, 7282; cds-WP_002894613.1, NZ_KK737547.1, 327943, 329055, -, 1113, 79; cds-WP _002894617.1, NZ_KK737547.1, 329055, 330956, -, 1902, 31380; cds-WP_002894620.1, NZ_KK737547.1, 330984, 331451, -, 468, 4607; cds-WP_002894623.1, NZ_KK737547.1, 331455, 331772, -, 318, 31; cds-WP _004151650.1, NZ_KK737547.1, 331995, 332624, -, 630, 279; cds-WP_004176879.1, NZ_KK737547.1, 332697, 333347, -, 651, 4285; cds-WP_032429857.1, NZ_KK737547.1, 333340, 334371, -, 1032, 105419; cds-WP _002894693.1, NZ_KK737547.1, 334371, 334961, -, 591, 5794; cds-WP _004147581.1, NZ_KK737547.1, 334976, 337558, -, 2583, 490179; cds-WP _032429977.1, NZ_KK737547.1, 337785, 338267, +, 483, 14; cds-WP_032429859.1, NZ_KK737547.1, 338313, 340106, -, 1794, 1581; cds-WP_032429860.1, NZ_KK737547.1, 340172, 341842, -, 1671, 977; cds-WP _002894706.1, NZ_KK737547.1, 342216, 342941, -, 726, 195; cds-WP _002894707.1, NZ_KK737547.1, 342941, 343615, -, 675, 628; cds-WP_002894709.1, NZ_KK737547.1, 343615, 344355, -, 741, 18338; cds-WP_002894712.1, NZ_KK737547.1, 344520, 345431, -, 912, 106501; cds-WP _032429861.1, NZ_KK737547.1, 345786, 347324, -, 1539, 23153; cds-WP_004197606.1, NZ_KK737547.1, 347450, 348328, -, 879, 39738; cds-WP_002894722.1, NZ_KK737547.1, 348476, 348949, -, 474, 1225; cds-WP_004176871.1, NZ_KK737547.1, 348946, 349992, -, 1047, 19147; cds-WP_002894730.1, NZ_KK737547.1, 350146, 351570, -, 1425, 44165; cds-WP _032429863.1, NZ_KK737547.1, 351750, 352925, +, 1176, 44; cds-WP_002894734.1, NZ_KK737547.1, 354033, 355697, -, 1665, 89732; cds-WP_002894738.1, NZ_KK737547.1, 355976, 356728, -, 753, 100847; cds-WP _002894742.1, NZ_KK737547.1, 356845, 358065, -, 1221, 9800; cds-WP_002894747.1, NZ_KK737547.1, 358074, 359222, -, 1149, 18951; cds-WP _002894749.1, NZ_KK737547.1, 359336, 360136, -, 801, 5998; cds-WP_032429865.1, NZ_KK737547.1, 360451, 362418, +, 1968, 4992; cds-WP_002894753.1, NZ_KK737547.1, 362597, 364264, +, 1668, 341659; cds-WP_004142270.1, NZ_KK737547.1, 364318, 364626, -, 309, 6; cds-WP _004152230.1, NZ_KK737547.1, 364699, 366102, +, 1404, 38; cds-WP_002894759.1, NZ_KK737547.1, 366150, 366482, +, 333, 2053; cds-WP_166686314.1, NZ_KK737547.1, 366534, 366977, -, 444, 549; cds-WP _002894761.1, NZ_KK737547.1, 367099, 367551, -, 453, 122525; cds-WP_002894765.1, NZ_KK737547.1, 367843, 368373, -, 531, 5353; cds-WP_002894767.1, NZ_KK737547.1, 368525, 368818, -, 294, 619; cds-WP_004152229.1, NZ_KK737547.1, 368952, 369725, -, 774, 22310; cds-WP _002894771.1, NZ_KK737547.1, 369909, 370457, +, 549, 59722; cds-WP _002894773.1, NZ_KK737547.1, 370490, 372130, +, 1641, 330165; cds-WP_004179052.1, NZ_KK737547.1, 372185, 372622, +, 438, 1239; cds-WP _002894776.1, NZ_KK737547.1, 372604, 373281, -, 678, 220; cds-WP _021441429.1, NZ_KK737547.1, 373278, 375965, -, 2688, 0; cds-WP _032429868.1, NZ_KK737547.1, 375966, 376541, -, 576, 16; cds-WP_021441430.1, NZ_KK737547.1, 376552, 378600, -, 2049, 0; cds-WP_004152226.1, NZ_KK737547.1, 378621, 380300, -, 1680, 0; cds-WP _020323459.1, NZ_KK737547.1, 380300, 380389, -, 90, 34; cds-WP _004142243.1, NZ_KK737547.1, 380696, 380905, +, 210, 0; cds-WP_032429869.1, NZ_KK737547.1, 381089, 382531, +, 1443, 7; cds-WP_004893379.1, NZ_KK737547.1, 382503, 383981, -, 1479, 0; cds-WP_002894919.1, NZ_KK737547.1, 384249, 384992, +, 744, 109; cds-WP _004176857.1, NZ_KK737547.1, 385007, 385663, +, 657, 19; cds-WP_004183433.1, NZ_KK737547.1, 385657, 386589, +, 933, 2036; cds-WP _004183435.1, NZ_KK737547.1, 386579, 387322, +, 744, 59; cds-WP _002894928.1, NZ_KK737547.1, 387404, 388129, +, 726, 4; cds-WP_004176855.1, NZ_KK737547.1, 388126, 389121, +, 996, 9714; cds-WP_004183438.1, NZ_KK737547.1, 389131, 389775, +, 645, 0; cds-WP_002894935.1, NZ_KK737547.1, 389791, 390582, +, 792, 48; cds-WP_004183439.1, NZ_KK737547.1, 390673, 391956, -, 1284, 1291721; cds-WP _004142216.1, NZ_KK737547.1, 392597, 393001, +, 405, 284571; cds-WP_002894949.1, NZ_KK737547.1, 392995, 393342, +, 348, 360445; cds-WP_004179067.1, NZ_KK737547.1, 393342, 395108, +, 1767, 1476851; cds-WP _002894954.1, NZ_KK737547.1, 395124, 395840, +, 717, 610782; cds-WP_012068464.1, NZ_KK737547.1, 396222, 399029, +, 2808, 2953128; cds-WP _004142209.1, NZ_KK737547.1, 399044, 400270, +, 1227, 547398; cds-WP _002895039.1, NZ_KK737547.1, 400374, 401540, +, 1167, 929955; cds-WP _002895043.1, NZ_KK737547.1, 401540, 402409, +, 870, 195922; cds-WP_002895047.1, NZ_KK737547.1, 403234, 404802, +, 1569, 137318; cds-WP _002895049.1, NZ_KK737547.1, 404818, 405957, +, 1140, 148537; cds-WP _002895051.1, NZ_KK737547.1, 405973, 406089, +, 117, 624; cds-WP_002895053.1, NZ_KK737547.1, 406089, 406379, +, 291, 46; cds-WP_002895056.1, NZ_KK737547.1, 406529, 406933, +, 405, 39927; cds-WP_004152221.1, NZ_KK737547.1, 406930, 407622, +, 693, 40909; cds-WP_002895061.1, NZ_KK737547.1, 407626, 408054, +, 429, 82; cds-WP _004179068.1, NZ_KK737547.1, 408154, 409479, +, 1326, 53812; cds-WP _032429872.1, NZ_KK737547.1, 409609, 410901, +, 1293, 253984; cds-WP _002895068.1, NZ_KK737547.1, 410936, 411460, +, 525, 370269; cds-WP _002895071.1, NZ_KK737547.1, 411470, 412264, +, 795, 261964; cds-WP_032409073.1, NZ_KK737547.1, 413744, 413893, -, 150, 0; cds-WP_002895074.1, NZ_KK737547.1, 413963, 415006, +, 1044, 95; cds-WP _004151689.1, NZ_KK737547.1, 415042, 415761, +, 720, 64; cds-WP_004147641.1, NZ_KK737547.1, 415758, 416702, -, 945, 48; cds-WP_002895084.1, NZ_KK737547.1, 416820, 417185, -, 366, 3; cds-WP_002895086.1, NZ_KK737547.1, 417500, 418552, +, 1053, 1639; cds-WP_002895089.1, NZ_KK737547.1, 418626, 419378, -, 753, 380666; cds-WP _023302155.1, NZ_KK737547.1, 419670, 420713, -, 1044, 2147; cds-WP _004179071.1, NZ_KK737547.1, 420707, 421855, -, 1149, 41273; cds-WP_004142166.1, NZ_KK737547.1, 421858, 422904, -, 1047, 27105; cds-WP _002895150.1, NZ_KK737547.1, 422914, 423930, -, 1017, 56353; cds-WP _032428718.1, NZ_KK737547.1, 424140, 425612, -, 1473, 62; cds-WP _032429873.1, NZ_KK737547.1, 425680, 426468, -, 789, 20; cds-WP_002895156.1, NZ_KK737547.1, 426621, 426770, +, 150, 1; cds-WP_032428721.1, NZ_KK737547.1, 426913, 427686, +, 774, 2825; cds-WP_002895159.1, NZ_KK737547.1, 427686, 428375, +, 690, 1166; cds-WP _004176832.1, NZ_KK737547.1, 428378, 429436, +, 1059, 166; cds-WP _002895164.1, NZ_KK737547.1, 429437, 430255, -, 819, 994; cds-WP_002895166.1, NZ_KK737547.1, 430484, 431479, +, 996, 203; cds-WP _002895168.1, NZ_KK737547.1, 431589, 431855, -, 267, 34169; cds-WP _004147654.1, NZ_KK737547.1, 431907, 432212, -, 306, 26147; cds-WP _004151693.1, NZ_KK737547.1, 432339, 432584, -, 246, 23955; cds-WP_004176831.1, NZ_KK737547.1, 432913, 434124, +, 1212, 98976;
cds-WP _032429874.1, NZ_KK737547.1, 434280, 435014, +, 735, 8465; cds-WP_004151695.1, NZ_KK737547.1, 435056, 435610, -, 555, 31; cds-WP_002895280.1, NZ_KK737547.1, 435692, 435904, -, 213, 231; cds-WP_032428722.1, NZ_KK737547.1, 436289, 436435, +, 147, 0; cds-WP _002895283.1, NZ_KK737547.1, 436640, 437146, +, 507, 0; cds-WP_072271782.1, NZ_KK737547.1, 437163, 438086, -, 924, 10; cds-AF52_RS0123875, NZ_KK737547.1, 438125, 441190, -, 3066, 1832; cds-WP_032429876.1, NZ_KK737547.1, 441191, 442276, -, 1086, 2; cds-WP _021441450.1, NZ_KK737547.1, 442810, 443241, +, 432, 0; cds-WP_020316497.1, NZ_KK737547.1, 443293, 445980, +, 2688, 11; cds-WP_004142122.1, NZ_KK737547.1, 446254, 447537, -, 1284, 18975; cds-WP_004182478.1, NZ_KK737547.1, 447692, 449011, +, 1320, 15524; cds-WP _021441452.1, NZ_KK737547.1, 449008, 449964, +, 957, 33665; cds-WP _002895358.1, NZ_KK737547.1, 450026, 450751, +, 726, 65001; cds-AF52_RS27965, NZ_KK737547.1, 450894, 451647, +, 754, 52370; cds-WP_160333877.1, NZ_KK737547.1, 451644, 452582, +, 939, 102950; cds-WP_032428723.1, NZ_KK737547.1, 452579, 454105, +, 1527, 34190; cds-WP_004147672.1, NZ_KK737547.1, 454204, 455586, +, 1383, 13159; cds-WP_002895575.1, NZ_KK737547.1, 456305, 456781, -, 477, 705; cds-WP_004223758.1, NZ_KK737547.1, 456852, 458141, -, 1290, 47; cds-WP _032429877.1, NZ_KK737547.1, 458355, 459395, +, 1041, 947; cds-WP_004183475.1, NZ_KK737547.1, 459392, 460549, +, 1158, 0; cds-WP_032429878.1, NZ_KK737547.1, 460533, 461288, +, 756, 588; cds-WP _002895648.1, NZ_KK737547.1, 461281, 462003, +, 723, 2; cds-WP_004152853.1, NZ_KK737547.1, 461945, 462667, -, 723, 911; cds-WP _002895652.1, NZ_KK737547.1, 462754, 462966, -, 213, 1; cds-WP _004147678.1, NZ_KK737547.1, 463378, 465399, +, 2022, 256; cds-WP_032429879.1, NZ_KK737547.1, 465648, 466745, -, 1098, 137; cds-WP _002895659.1, NZ_KK737547.1, 466749, 467420, -, 672, 5; cds-WP _021441454.1, NZ_KK737547.1, 467420, 469060, -, 1641, 11; cds-WP_004142092.1, NZ_KK737547.1, 469165, 470070, -, 906, 3155; cds-WP_002895665.1, NZ_KK737547.1, 470405, 471394, +, 990, 11156; cds-WP_021441455.1, NZ_KK737547.1, 471418, 471930, +, 513, 13630; cds-WP _002895670.1, NZ_KK737547.1, 471934, 472422, +, 489, 2; cds-WP_002895672.1, NZ_KK737547.1, 472415, 472663, +, 249, 3; cds-WP _002895674.1, NZ_KK737547.1, 472665, 473117, +, 453, 987; cds-WP _004142079.1, NZ_KK737547.1, 473163, 473867, +, 705, 1041; cds-WP_032429880.1, NZ_KK737547.1, 474048, 475013, -, 966, 4; cds-WP_032429882.1, NZ_KK737547.1, 475010, 476254, -, 1245, 3; cds-WP_004179098.1, NZ_KK737547.1, 476251, 477012, -, 762, 7; cds-WP_004176793.1, NZ_KK737547.1, 477144, 477581, +, 438, 4287; cds-WP_004142068.1, NZ_KK737547.1, 477642, 478748, -, 1107, 9424; cds-WP_004212202.1, NZ_KK737547.1, 478878, 480011, -, 1134, 81; cds-WP_032429883.1, NZ_KK737547.1, 480001, 481740, -, 1740, 0; cds-WP _002895745.1, NZ_KK737547.1, 481733, 482728, -, 996, 25; cds-WP_002895746.1, NZ_KK737547.1, 482728, 483387, -, 660, 0; cds-WP_004223785.1, NZ_KK737547.1, 483461, 484384, -, 924, 19456; cds-WP_004142057.1, NZ_KK737547.1, 484376, 484513, +, 138, 0; cds-WP _019705008.1, NZ_KK737547.1, 484801, 486132, +, 1332, 0; cds-AF52_RS27125, NZ_KK737547.1, 486247, 486348, -, 102, 157; cds-WP_032429885.1, NZ_KK737547.1, 488364, 488701, -, 338, 275; cds-WP_002895750.1, NZ_KK737547.1, 488735, 489484, +, 750, 0; cds-WP_002895753.1, NZ_KK737547.1, 489498, 490343, +, 846, 4; cds-WP_002895757.1, NZ_KK737547.1, 490343, 491335, +, 993, 0; cds-WP_032429886.1, NZ_KK737547.1, 491550, 492905, +, 1356, 121; cds-WP_004147693.1, NZ_KK737547.1, 493093, 495249, +, 2157, 48468; cds-WP_004179105.1, NZ_KK737547.1, 495279, 496247, +, 969, 1179; cds-WP _004176772.1, NZ_KK737547.1, 496357, 496617, -, 261, 646; cds-WP_032428726.1, NZ_KK737547.1, 496903, 497169, -, 267, 0; cds-WP_004183485.1, NZ_KK737547.1, 497190, 497450, -, 261, 2; cds-WP _016529094.1, NZ_KK737547.1, 497590, 498204, -, 615, 896; cds-WP_021441457.1, NZ_KK737547.1, 498301, 499215, +, 915, 16; cds-WP _021441458.1, NZ_KK737547.1, 499218, 501413, -, 2196, 5919; cds-WP_004142040.1, NZ_KK737547.1, 501525, 502247, -, 723, 21863; cds-WP_002895837.1, NZ_KK737547.1, 502244, 502903, -, 660, 48047; cds-WP_002895839.1, NZ_KK737547.1, 503029, 503775, -, 747, 258478; cds-WP_002895841.1, NZ_KK737547.1, 504184, 504687, -, 504, 484; cds-WP_002895842.1, NZ_KK737547.1, 504925, 505812, -, 888, 0; cds-WP _002895845.1, NZ_KK737547.1, 506164, 506676, +, 513, 27120; cds-WP_021441460.1, NZ_KK737547.1, 506767, 508347, -, 1581, 79450; cds-WP _002895851.1, NZ_KK737547.1, 508616, 508741, -, 126, 2386; cds-WP _004176765.1, NZ_KK737547.1, 508927, 509400, +, 474, 12; cds-WP _021441462.1, NZ_KK737547.1, 509397, 510509, +, 1113, 55; cds-WP _004176762.1, NZ_KK737547.1, 510679, 512055, +, 1377, 2653; cds-WP_004151705.1, NZ_KK737547.1, 512124, 512840, +, 717, 1294; cds-WP _002895871.1, NZ_KK737547.1, 512986, 513900, -, 915, 80712; cds-WP _002895874.1, NZ_KK737547.1, 514101, 514886, -, 786, 431; cds-WP _002895876.1, NZ_KK737547.1, 515065, 516657, +, 1593, 11422; cds-WP _021441465.1, NZ_KK737547.1, 517192, 519555, -, 2364, 2; cds-WP _004183491.1, NZ_KK737547.1, 519571, 520872, -, 1302, 0; cds-WP _004219745.1, NZ_KK737547.1, 521115, 522245, +, 1131, 835; cds-WP_004176744.1, NZ_KK737547.1, 522242, 523507, -, 1266, 11; cds-WP _004212174.1, NZ_KK737547.1, 523647, 524462, -, 816, 5868; cds-WP _032429888.1, NZ_KK737547.1, 524639, 527071, -, 2433, 1; cds-WP_004183492.1, NZ_KK737547.1, 527076, 527975, -, 900, 0; cds-WP_004141998.1, NZ_KK737547.1, 528130, 528885, -, 756, 27; cds-WP _032429890.1, NZ_KK737547.1, 528885, 530120, -, 1236, 30; cds-WP _004147725.1, NZ_KK737547.1, 530356, 531297, +, 942, 237; cds-WP _004151710.1, NZ_KK737547.1, 531315, 533174, +, 1860, 479; cds-WP _012068490.1, NZ_KK737547.1, 533207, 534748, +, 1542, 7331; cds-WP _002892997.1, NZ_KK737547.1, 534795, 535715, +, 921, 9715; cds-WP _004147729.1, NZ_KK737547.1, 535717, 536628, +, 912, 4202; cds-WP _032429891.1, NZ_KK737547.1, 537149, 538474, -, 1326, 206050; cds-WP _004176729.1, NZ_KK737547.1, 538707, 539948, -, 1242, 61870; cds-WP_004151712.1, NZ_KK737547.1, 540327, 540710, +, 384, 22377; cds-WP_029884283.1, NZ_KK737547.1, 540826, 541935, +, 1110, 982; cds-WP_004176727.1, NZ_KK737547.1, 541938, 542564, -, 627, 3717; cds-AF52_RS27135, NZ_KK737547.1, 542764, 542887, -, 124, 309; cds-WP_171290763.1, NZ_KK737547.1, 551069, 551268, -, 200, 532; cds-WP_179139609.1, NZ_KK737547.1, 552619, 552832, +, 214, 441; cds-AF52_RS11970, NZ_KK737547.1, 559347, 559565, +, 219, 735; cds-WP _032428727.1, NZ_KK737547.1, 559740, 559868, +, 129, 13; cds-WP_032429893.1, NZ_KK737547.1, 560520, 561722, +, 1203, 37496; cds-WP_004151717.1, NZ_KK737547.1, 561761, 562519, -, 759, 7828; cds-WP _004141974.1, NZ_KK737547.1, 562621, 563214, -, 594, 24; cds-WP_004141972.1, NZ_KK737547.1, 563542, 564774, +, 1233, 526; cds-WP _004141971.1, NZ_KK737547.1, 564812, 565624, -, 813, 162; cds-WP_004151719.1, NZ_KK737547.1, 565624, 566826, -, 1203, 1445; cds-WP _032429895.1, NZ_KK737547.1, 566909, 567472, +, 564, 5; cds-WP _032429979.1, NZ_KK737547.1, 567650, 569335, -, 1686, 51; cds-WP _002896351.1, NZ_KK737547.1, 569602, 569985, +, 384, 449; cds-WP_002896352.1, NZ_KK737547.1, 569992, 570255, -, 264, 4074; cds-WP_004179132.1, NZ_KK737547.1, 570458, 570745, +, 288, 3587; cds-WP_004179133.1, NZ_KK737547.1, 570729, 571451, +, 723, 2; cds-WP_002896363.1, NZ_KK737547.1, 571566, 572468, +, 903, 352; cds-WP_002896365.1, NZ_KK737547.1, 572557, 573036, +, 480, 10803; cds-WP _002896368.1, NZ_KK737547.1, 573385, 574497, +, 1113, 10251; cds-WP_002896370.1, NZ_KK737547.1, 574661, 575794, +, 1134, 13124; cds-WP _002896371.1, NZ_KK737547.1, 575805, 576758, +, 954, 34; cds-WP _002896372.1, NZ_KK737547.1, 576755, 577600, +, 846, 2; cds-WP_002896376.1, NZ_KK737547.1, 577658, 578146, +, 489, 2; cds-WP _004147758.1, NZ_KK737547.1, 578188, 579315, +, 1128, 3930; cds-WP _002896380.1, NZ_KK737547.1, 579394, 580110, +, 717, 4893; cds-WP_002896382.1, NZ_KK737547.1, 580107, 581579, +, 1473, 4; cds-WP _002896384.1, NZ_KK737547.1, 581666, 582397, -, 732, 1440; cds-WP_004141917.1, NZ_KK737547.1, 582581, 583249, -, 669, 35209; cds-WP_002896386.1, NZ_KK737547.1, 583249, 583965, -, 717, 17979; cds-WP_002896390.1, NZ_KK737547.1, 583972, 584703, -, 732, 788; cds-WP_002896392.1, NZ_KK737547.1, 584724, 585452, -, 729, 215; cds-WP _002896394.1, NZ_KK737547.1, 585679, 586194, -, 516, 1240; cds-AF52_RS11825, NZ_KK737547.1, 587073, 587345, +, 273, 142; cds-WP_013815099.1-37, NZ_KK737547.1, 587378, 588346, +, 969, 3269; cds-AF52_RS11815, NZ_KK737547.1, 588400, 589278, +, 879, 93; cds-WP_032429899.1, NZ_KK737547.1, 589310, 590140, +, 831, 23377; cds-WP _002896399.1, NZ_KK737547.1, 590137, 591150, -, 1014, 9698; cds-WP_004209682.1, NZ_KK737547.1, 591238, 592680, -, 1443, 39330; cds-WP_032429901.1, NZ_KK737547.1, 592691, 593692, -, 1002, 37375; cds-WP_004176700.1, NZ_KK737547.1, 593731, 595449, -, 1719, 25; cds-WP _002896408.1, NZ_KK737547.1, 595601, 596035, +, 435, 0; cds-WP _004147771.1, NZ_KK737547.1, 596247, 597215, -, 969, 0; cds-WP_004176696.1, NZ_KK737547.1, 597226, 598878, -, 1653, 1; cds-AF52_RS11770, NZ_KK737547.1, 599022, 599681, -, 660, 0; cds-WP_013815099.1-38, NZ_KK737547.1, 599738, 600706, -, 969, 4863; cds-AF52_RS11760, NZ_KK737547.1, 600739, 600987, -, 249, 167; cds-WP _032429902.1, NZ_KK737547.1, 601103, 601798, -, 696, 9; cds-WP_013815099.1-39, NZ_KK737547.1, 602061, 603029, -, 969, 4723; cds-WP_032429903.1, NZ_KK737547.1, 603283, 604941, +, 1659, 3658; cds-WP _002896440.1, NZ_KK737547.1, 605088, 606203, +, 1116, 50; cds-WP_032429904.1, NZ_KK737547.1, 606200, 608140, +, 1941, 5; cds-WP _002896516.1, NZ_KK737547.1, 608217, 608438, -, 222, 14357; cds-WP_002896520.1, NZ_KK737547.1, 608764, 609081, +, 318, 160863; cds-WP _002896522.1, NZ_KK737547.1, 609112, 611391, +, 2280, 228557; cds-WP_001040187.1, NZ_KK737547.1, 611511, 611729, -, 219, 19295; cds-WP _002898014.1, NZ_KK737547.1, 612083, 612784, -, 702, 15541; cds-WP_004224003.1, NZ_KK737547.1, 612829, 614550, -, 1722, 2306; cds-WP_032429906.1, NZ_KK737547.1, 614551, 616317, -, 1767,9299; cds-WP_002898019.1, NZ_KK737547.1, 616432, 617400, -, 969, 24905; cds-WP_000228469.1, NZ_KK737547.1, 617933, 618427, +, 495, 5107; cds-AF52_RS0123915, NZ_KK737547.1, 618563, 622808, +, 4246, 66886; cds-WP_002898132.1, NZ_KK737547.1, 622931, 623542, +, 612, 311; cds-WP_004179165.1, NZ_KK737547.1, 623551, 624894, +, 1344, 7408; cds-WP _002898139.1, NZ_KK737547.1, 624985, 626277, +, 1293, 117868; cds-WP_004147794.1, NZ_KK737547.1, 626478, 628916, +, 2439, 0; cds-WP_004150845.1, NZ_KK737547.1, 628927, 629544, +, 618, 0; cds-WP_004150844.1, NZ_KK737547.1, 629546, 630409, +, 864, 0; cds-WP_032429909.1, NZ_KK737547.1, 630684, 631832, +, 1149, 6384; cds-WP _002898145.1, NZ_KK737547.1, 631995, 632735, -, 741, 3; cds-WP_002898148.1, NZ_KK737547.1, 632927, 635209, -, 2283, 189377; cds-WP_032429911.1, NZ_KK737547.1, 635261, 636118, -, 858, 5352; cds-WP_004150842.1, NZ_KK737547.1, 636504, 638264, -, 1761, 15462; cds-WP_002898152.1, NZ_KK737547.1, 638380, 639072, +, 693, 3940; cds-WP _004176687.1, NZ_KK737547.1, 639261, 640349, +, 1089, 560; cds-WP_002898157.1, NZ_KK737547.1, 640423, 641706, +, 1284, 23; cds-WP_002898160.1, NZ_KK737547.1, 641881, 642564, +, 684, 73377; cds-WP_002898162.1, NZ_KK737547.1, 642698, 644371, +, 1674, 2929632; cds-WP _002898165.1, NZ_KK737547.1, 644523, 644810, +, 288, 23616; cds-WP_032411196.1, NZ_KK737547.1, 645016, 647280, +, 2265, 0; cds-WP_004201387.1, NZ_KK737547.1, 647317, 649065, +, 1749, 12139; cds-WP_016946992.1, NZ_KK737547.1, 649062, 650042, +, 981, 13; cds-WP_032429912.1, NZ_KK737547.1, 650097, 651326, +, 1230, 100; cds-WP _002898177.1, NZ_KK737547.1, 651390, 651572, +, 183, 8666; cds-WP_032429914.1, NZ_KK737547.1, 651569, 652315, +, 747, 41024; cds-WP _002898182.1, NZ_KK737547.1, 652453, 653346, +, 894, 0; cds-WP_004141786.1, NZ_KK737547.1, 653323, 654102, -, 780, 2; cds-WP _002898187.1, NZ_KK737547.1, 654224, 655024, +, 801, 10735; cds-WP_032429915.1, NZ_KK737547.1, 655021, 656343, +, 1323, 16514; cds-WP_004150840.1, NZ_KK737547.1, 656324, 657028, +, 705, 216; cds-WP_004176682.1, NZ_KK737547.1, 657028, 661476, +, 4449, 216607; cds-WP _002898195.1, NZ_KK737547.1, 661677, 663467, +, 1791, 1112; cds-WP _002898198.1, NZ_KK737547.1, 663770, 664321, +, 552, 195539; cds-WP _002898200.1, NZ_KK737547.1, 664335, 664982, +, 648, 392; cds-WP _002898202.1, NZ_KK737547.1, 665051, 666241, -, 1191, 62998; cds-WP_004141771.1, NZ_KK737547.1, 666429, 667508, -, 1080, 106019; cds-
WP_032429917.1, NZ_KK737547.1, 668100, 669500, -, 1401, 126913; cds-AF52_RS11510, NZ_KK737547.1, 669581, 670140, -, 560, 1; cds-WP _013815099.1-40, NZ_KK737547.1, 670203, 671171, -, 969, 5124; cds-WP_004179185.1, NZ_KK737547.1, 671515, 672726, +, 1212, 1; cds-WP_023286202.1, NZ_KK737547.1, 672849, 674138, +, 1290, 0; cds-WP_032429918.1, NZ_KK737547.1, 674163, 675401, +, 1239, 5; cds-WP _004179190.1, NZ_KK737547.1, 675420, 676586, +, 1167, 16; cds-WP_002898217.1, NZ_KK737547.1, 676629, 677831, -, 1203, 2040; cds-WP _002898220.1, NZ_KK737547.1, 678158, 680773, +, 2616, 171608; cds-WP _004150838.1, NZ_KK737547.1, 680978, 681751, -, 774, 5427; cds-WP_004141755.1, NZ_KK737547.1, 681748, 682539, -, 792, 11; cds-WP _002898293.1, NZ_KK737547.1, 682550, 683695, -, 1146, 0; cds-WP _023284918.1, NZ_KK737547.1, 683692, 684654, -, 963, 0; cds-WP _032429920.1, NZ_KK737547.1, 684647, 685222, -, 576, 0; cds-WP _002898303.1, NZ_KK737547.1, 685472, 686482, +, 1011, 1955; cds-WP_002898308.1, NZ_KK737547.1, 686652, 687194, +, 543, 10627; cds-WP_004179196.1, NZ_KK737547.1, 687191, 688300, -, 1110, 1098; cds-WP _004147847.1, NZ_KK737547.1, 688400, 690505, +, 2106, 3812; cds-WP_004147848.1, NZ_KK737547.1, 690518, 692425, +, 1908, 12611; cds-WP_004147849.1, NZ_KK737547.1, 692440, 693708, +, 1269, 13325; cds-WP_002898390.1, NZ_KK737547.1, 693698, 695335, +, 1638, 1035; cds-WP _002898392.1, NZ_KK737547.1, 695335, 695898, +, 564, 239; cds-WP _002898396.1, NZ_KK737547.1, 696152, 696319, +, 168, 4; cds-WP _002898398.1, NZ_KK737547.1, 696388, 696906, -, 519, 3863; cds-WP_032429922.1, NZ_KK737547.1, 696976, 698733, -, 1758, 109687; cds-WP_002898404.1, NZ_KK737547.1, 698919, 699371, +, 453, 65; cds-WP _002898408.1, NZ_KK737547.1, 699503, 700573, -, 1071, 2138616; cds-WP_002898418.1, NZ_KK737547.1, 700926, 701435, -, 510, 169; cds-WP_032429924.1, NZ_KK737547.1, 701784, 702380, +, 597, 4; cds-WP_002898422.1, NZ_KK737547.1, 702394, 704529, -, 2136, 2163; cds-WP_002898425.1, NZ_KK737547.1, 704547, 704993, -, 447, 199; cds-WP_004176653.1, NZ_KK737547.1, 705118, 707172, +, 2055, 3801; cds-WP_002898432.1, NZ_KK737547.1, 707184, 707642, -, 459, 366; cds-WP _004144085.1, NZ_KK737547.1, 707797, 708210, +, 414, 690; cds-WP _013815099.1-41, NZ_KK737547.1, 708377, 709345, -, 969, 4742; cds-AF52_RS0123935, NZ_KK737547.1, 709895, 712733, +, 2839, 58718; cds-WP _072001581.1, NZ_KK737547.1, 724264, 728283, +, 4020, 17188; cds-AF52_RS11335, NZ_KK737547.1, 728452, 729213, +, 762, 0; cds-AF52_RS27170, NZ_KK737547.1, 729248, 729418, +, 171, 364; cds-AF52_RS11330, NZ_KK737547.1, 730319, 730471, +, 153, 232; cds-AF52_RS11325, NZ_KK737547.1, 730530, 733256, +, 2727, 0; cds-WP_032429986.1, NZ_KK737547.1, 733403, 734278, +, 876, 0; cds-WP _002898441.1, NZ_KK737547.1, 734321, 734638, -, 318, 2545; cds-WP_032429928.1, NZ_KK737547.1, 734698, 735900, -, 1203, 508; cds-WP _004210995.1, NZ_KK737547.1, 736091, 736645, +, 555, 825; cds-WP_004183586.1, NZ_KK737547.1, 736663, 737451, +, 789, 897; cds-WP _032429987.1, NZ_KK737547.1, 737500, 737781, +, 282, 0; cds-WP _002898457.1, NZ_KK737547.1, 737778, 738107, -, 330, 2241; cds-WP _002898458.1, NZ_KK737547.1, 738196, 738855, -, 660, 61435; cds-WP _032429929.1, NZ_KK737547.1, 739125, 740777, +, 1653, 152; cds-AF52_RS26415, NZ_KK737547.1, 741134, 741886, -, 753, 1; cds-WP_032429931.1, NZ_KK737547.1, 741867, 742055, +, 189, 0; cds-WP _032429933.1, NZ_KK737547.1, 742225, 743190, -, 966, 27; cds-WP_004211006.1, NZ_KK737547.1, 743248, 744696, -, 1449, 221; cds-WP_002898571.1, NZ_KK737547.1, 744715, 745839, -, 1125, 3559; cds-WP _009308376.1, NZ_KK737547.1, 745876, 747483, -, 1608, 55552; cds-WP _032429935.1, NZ_KK737547.1, 747988, 749073, -, 1086, 0; cds-WP _004221441.1, NZ_KK737547.1, 749156, 750118, +, 963, 10; cds-WP_002898585.1, NZ_KK737547.1, 750115, 751395, -, 1281, 577; cds-WP_032429937.1, NZ_KK737547.1, 751398, 752885, -, 1488, 10227; cds-WP_002898590.1, NZ_KK737547.1, 753206, 753763, -, 558, 182883; cds-WP_020802183.1, NZ_KK737547.1, 753789, 754541, -, 753, 30701; cds-WP_002898595.1, NZ_KK737547.1, 754767, 756188, +, 1422, 71874; cds-WP _004141463.1, NZ_KK737547.1, 756542, 757933, +, 1392, 62263; cds-WP _002898600.1, NZ_KK737547.1, 758052, 758174, -, 123, 1257; cds-WP_021441517.1, NZ_KK737547.1, 758431, 758652, +, 222,1; cds-WP_004150831.1, NZ_KK737547.1, 758673, 759461, -, 789, 577; cds-WP_004141456.1, NZ_KK737547.1, 759577, 760026, -, 450, 0; cds-WP_004183618.1, NZ_KK737547.1, 760179, 761426, +, 1248, 36170; cds-WP_002898701.1, NZ_KK737547.1, 761495, 761722, -, 228, 41; cds-WP_002898704.1, NZ_KK737547.1, 761743, 762339, -, 597, 26; cds-WP_004224058.1, NZ_KK737547.1, 762556, 762687, -, 132, 0; cds-WP_002898708.1, NZ_KK737547.1, 762775, 762948, +, 174, 0; cds-WP _004199515.1, NZ_KK737547.1, 763111, 764049, +, 939, 0; cds-WP_004191019.1, NZ_KK737547.1, 764455, 765777, -, 1323, 0; cds-WP _004176611.1, NZ_KK737547.1, 765799, 766293, -, 495, 0; cds-WP _004141442.1, NZ_KK737547.1, 766304, 766894, -, 591, 0; cds-WP_004179289.1, NZ_KK737547.1, 766891, 767694, -, 804, 0; cds-WP _002898877.1, NZ_KK737547.1, 767691, 768083, -, 393, 0; cds-WP_004224061.1, NZ_KK737547.1, 768076, 768786, -, 711, 0; cds-WP _004179293.1, NZ_KK737547.1, 768786, 769877, -, 1092, 0; cds-WP _002898883.1, NZ_KK737547.1, 770162, 770800, +, 639, 17; cds-WP _002898886.1, NZ_KK737547.1, 770797, 771192, -, 396, 0; cds-WP_032429939.1, NZ_KK737547.1, 771378, 775340, -, 3963, 171600; cds-WP_013815099.1-42, NZ_KK737547.1, 775708, 776676, +, 969, 8388; cds-WP_032429941.1, NZ_KK737547.1, 776720, 777919, +, 1200, 7166; cds-AF52_RS0123960, NZ_KK737547.1, 777953, 778729, +, 777, 2; cds-WP _020806188.1-3, NZ_KK737547.1, 778830, 779810, -, 981, 5328; cds-WP _032429943.1, NZ_KK737547.1, 779855, 781216, +, 1362, 0; cds-WP_021440205.1, NZ_KK737547.1, 781216, 781701, +, 486, 0; cds-WP_021440204.1, NZ_KK737547.1, 781711, 782727, +, 1017, 0; cds-WP _004143751.1, NZ_KK737547.1, 782737, 783510, +, 774, 0; cds-WP _002906464.1, NZ_KK737547.1, 783507, 784385, -, 879, 1; cds-WP_148673683.1, NZ_KK737547.1, 784611, 787658, +, 3048, 3592; cds-WP_004143747.1, NZ_KK737547.1, 787670, 788554, +, 885, 0; cds-WP_002906457.1, NZ_KK737547.1, 788547, 789203, +, 657, 0; cds-AF52_RS25665, NZ_KK737547.1, 789256, 789581, -, 326, 0; cds-WP_004175994.1, NZ_KK737547.1, 789578, 789763, -, 186, 0; cds-WP _004180084.1, NZ_KK737547.1, 789942, 790760, -, 819, 0; cds-WP _021440202.1, NZ_KK737547.1, 790828, 791268, -, 441, 0; cds-WP_004180082.1, NZ_KK737547.1, 791533, 792687, +, 1155, 1632; cds-WP_020802157.1, NZ_KK737547.1, 792694, 793785, -, 1092, 0; cds-WP_004184178.1, NZ_KK737547.1, 793795, 794781, -, 987, 0; cds-WP_021440201.1, NZ_KK737547.1, 794781, 796397, -, 1617, 0; cds-WP _020324770.1, NZ_KK737547.1, 796394, 797242, -, 849, 2; cds-WP_023280140.1, NZ_KK737547.1, 797239, 798183, -, 945, 0; cds-WP_002906226.1, NZ_KK737547.1, 798194, 799816, -, 1623, 0; cds-WP_002906221.1, NZ_KK737547.1, 799986, 800996, -, 1011, 63967; cds-WP _002906218.1, NZ_KK737547.1, 801249, 801848, +, 600, 0; cds-WP _004176019.1, NZ_KK737547.1, 802022, 802975, +, 954, 132; cds-WP_032428292.1, NZ_KK737547.1, 803012, 804727, -, 1716, 3281; cds-WP_004200032.1, NZ_KK737547.1, 805090, 805776, -, 687, 5; cds-WP _021440197.1, NZ_KK737547.1, 805773, 806468, -, 696, 0; cds-WP_002906125.1, NZ_KK737547.1, 806652, 808349, -, 1698, 94103; cds-WP _002906122.1, NZ_KK737547.1, 808406, 808534, +, 129, 255; cds-WP_004143727.1, NZ_KK737547.1, 808531, 808671, -, 141, 0; cds-WP _032429948.1, NZ_KK737547.1, 808849, 809700, -, 852, 0; cds-WP_021440195.1, NZ_KK737547.1, 809814, 810707, +, 894, 0; cds-WP _004180069.1, NZ_KK737547.1, 810775, 811020, -, 246, 1; cds-WP _004151242.1, NZ_KK737547.1, 811217, 812131, -, 915, 10; cds-WP _004143718.1, NZ_KK737547.1, 812787, 812972, +, 186, 1; cds-WP_013815099.1-43, NZ_KK737547.1, 813024, 813992, +, 969, 5839; cds-WP _004143717.1, NZ_KK737547.1, 814404, 814619, +, 216, 0; cds-WP _023301896.1, NZ_KK737547.1, 814643, 815149, +, 507, 0; cds-WP _004143715.1, NZ_KK737547.1, 815166, 815348, +, 183, 0; cds-WP _021440193.1, NZ_KK737547.1, 815388, 817013, +, 1626, 0; cds-WP _032429949.1, NZ_KK737547.1, 817010, 818005, -, 996, 0; cds-WP _004148482.1, NZ_KK737547.1, 818324, 818770, -, 447, 0; cds-WP _021440191.1, NZ_KK737547.1, 818779, 819282, -, 504, 0; cds-WP_002906035.1, NZ_KK737547.1, 819427, 819831, +, 405, 1; cds-WP_002906033.1, NZ_KK737547.1, 819847, 819993, +, 147, 0; cds-WP _002906030.1, NZ_KK737547.1, 820107, 820538, +, 432, 2; cds-WP _021440189.1, NZ_KK737547.1, 820643, 821512, +, 870, 0; cds-WP_004184165.1, NZ_KK737547.1, 821514, 822200, -, 687, 349; cds-WP_004143707.1, NZ_KK737547.1, 822190, 822372, -, 183, 53; cds-WP _004151892.1, NZ_KK737547.1, 822422, 823345, +, 924, 0; cds-WP _023282626.1, NZ_KK737547.1, 823595, 824512, +, 918, 110; cds-WP _023280134.1, NZ_KK737547.1, 824828, 827128, +, 2301, 565; cds-WP_002906016.1, NZ_KK737547.1, 827278, 828159, +, 882, 1; cds-WP _004180040.1, NZ_KK737547.1, 828422, 829828, -, 1407, 388; cds-WP _002906011.1, NZ_KK737547.1, 830416, 831096, +, 681, 2; cds-AF52_RS0123990, NZ_KK737547.1, 831194, 832255, +, 1062, 7571; cds-WP_004180028.1, NZ_KK737547.1, 832290, 834467, -, 2178, 17731; cds-WP_004184162.1, NZ_KK737547.1, 834736, 835668, -, 933, 13; cds-WP_002905764.1, NZ_KK737547.1, 835768, 836202, +, 435, 0; cds-WP_004143692.1, NZ_KK737547.1, 836212, 836688, +, 477, 0; cds-WP_002905759.1, NZ_KK737547.1, 836887, 838797, +, 1911, 28; cds-WP _004189919.1, NZ_KK737547.1, 838910, 839647, +, 738, 0; cds-WP _004189922.1, NZ_KK737547.1, 839644, 841929, -, 2286, 0; cds-WP _004184158.1, NZ_KK737547.1, 841929, 843071, -, 1143, 5; cds-WP_004143686.1, NZ_KK737547.1, 843058, 843336, -, 279, 0; cds-WP_004143685.1, NZ_KK737547.1, 843339, 844094, -, 756, 0; cds-WP_004148467.1, NZ_KK737547.1, 844088, 845014, -, 927, 43; cds-WP _002905689.1, NZ_KK737547.1, 845069, 845140, -, 72, 5; cds-WP_021440182.1, NZ_KK737547.1, 845277, 846296, -, 1020, 128; cds-WP _032429953.1, NZ_KK737547.1, 846539, 847531, +, 993, 0; cds-WP_004176065.1, NZ_KK737547.1, 847618, 848358, +, 741, 0; cds-WP _032428291.1, NZ_KK737547.1, 848355, 849698, +, 1344, 0; cds-WP_004184155.1, NZ_KK737547.1, 849801, 850991, +, 1191, 1; cds-WP_032429955.1, NZ_KK737547.1, 851905, 852798, -, 894, 11; cds-WP_187079193.1, NZ_KK737547.1, 852992, 854473, -, 1482, 131945; cds-WP_002905540.1, NZ_KK737547.1, 854483, 855868, -, 1386, 23858; cds-WP_013815099.1-44, NZ_KK737547.1, 856165, 857133, +, 969, 3991; cds-WP _021440179.1, NZ_KK737547.1, 857241, 858176, -, 936, 10882; cds-WP_032429957.1, NZ_KK737547.1, 858201, 858941, -, 741, 3464; cds-WP_004189949.1, NZ_KK737547.1, 858938, 859666, -, 729, 80; cds-WP_004189951.1, NZ_KK737547.1, 859663, 860358, -, 696, 27706; cds-AF52_RS28000, NZ_KK737547.1, 860529, 860615, +, 87, 0; cds-WP _002905531.1, NZ_KK737547.1, 860775, 861881, +, 1107, 0; cds-WP _021440176.1, NZ_KK737547.1, 861919, 863091, -, 1173, 10460; cds-WP _032411533.1, NZ_KK737547.1, 863105, 864028, -, 924, 384; cds-WP_004148452.1, NZ_KK737547.1, 864221, 865261, -, 1041, 1; cds-WP _032429958.1, NZ_KK737547.1, 865660, 866916, +, 1257, 19582; cds-WP _021440173.1, NZ_KK737547.1, 867015, 868325, -, 1311, 3342; cds-WP _021440172.1, NZ_KK737547.1, 868427, 869923, -, 1497, 0; cds-WP_004179987.1, NZ_KK737547.1, 870068, 870784, +, 717, 23; cds-AF52_RS10695, NZ_KK737547.1, 870784, 871655, +, 872, 0; cds-WP_004179983.1, NZ_KK737547.1, 871540, 872472, +, 933, 9134; cds-WP _004184144.1, NZ_KK737547.1, 872561, 873352, -, 792, 0; cds-WP_004189983.1, NZ_KK737547.1, 873462, 874229, +, 768, 251; cds-WP _004176101.1, NZ_KK737547.1, 874432, 874656, +, 225, 1; cds-AF52_RS25690, NZ_KK737547.1, 874992, 875342, +, 351, 26; cds-WP _002882540.1, NZ_KK737548.1, 3401, 4102, +, 702, 237; cds-WP _002882539.1, NZ_KK737548.1, 4115, 5512, +, 1398, 1639; cds-WP_004187944.1, NZ_KK737548.1, 5509, 6501, -, 993, 4725; cds-WP_187079194.1, NZ_KK737548.1, 6505, 7437, -, 933, 44; cds-WP _002882536.1, NZ_KK737548.1, 7538, 8428, -, 891, 9319; cds-WP_002882531.1, NZ_KK737548.1, 8456, 9421, -, 966, 16; cds-WP _002882529.1, NZ_KK737548.1, 9427, 10932, -, 1506, 57; cds-WP_002882527.1, NZ_KK737548.1, 10943, 11362, -, 420, 331; cds-WP _002882520.1, NZ_KK737548.1, 11548, 13416, -, 1869, 74054; cds-WP _004151555.1, NZ_KK737548.1, 13634, 15133, +, 1500, 7228; cds-WP_002882517.1, NZ_KK737548.1, 15130, 16578, +, 1449, 1439; cds-WP _002882514.1, NZ_KK737548.1, 16582, 17574, -, 993, 393; cds-WP_002882510.1, NZ_KK737548.1, 17726, 18184, +, 459, 20; cds-WP _004144988.1, NZ_KK737548.1, 18283, 18723, +, 441, 1671; cds-WP_004144989.1, NZ_KK737548.1, 19105,
20994, +, 1890, 61084; cds-WP_004151554.1, NZ_KK737548.1, 21110, 21733, +, 624, 143; cds-WP _013815099.1-45, NZ_KK737548.1, 22264, 23232, +, 969, 5906; cds-WP_004144991.1, NZ_KK737548.1, 23416, 23796, +, 381, 30511; cds-WP _004151552.1, NZ_KK737548.1, 23804, 24619, +, 816, 218156; cds-WP_000429386.1, NZ_KK737548.1, 24669, 24908, +, 240, 196443; cds-WP _004107293.1, NZ_KK737548.1, 24959, 25429, +, 471, 442517; cds-WP _004144994.1, NZ_KK737548.1, 25444, 25977, +, 534, 309143; cds-WP_004144995.1, NZ_KK737548.1, 25990, 27531, +, 1542, 1079801; cds-WP _004144996.1, NZ_KK737548.1, 27582, 28445, +, 864, 775955; cds-WP _004144997.1, NZ_KK737548.1, 28472, 29854, +, 1383, 1371647; cds-WP_001251971.1, NZ_KK737548.1, 29875, 30294, +, 420, 176446; cds-WP _004151550.1, NZ_KK737548.1, 30991, 32361, +, 1371, 33899; cds-WP_004150309.1, NZ_KK737548.1, 32545, 34374, +, 1830, 95722; cds-WP_004150308.1, NZ_KK737548.1, 34711, 35751, +, 1041, 393; cds-WP _004145004.1, NZ_KK737548.1, 35880, 36839, +, 960, 61511; cds-WP_004151547.1, NZ_KK737548.1, 36839, 37729, +, 891, 81964; cds-WP _004145006.1, NZ_KK737548.1, 37777, 38550, +, 774, 26945; cds-WP _004145007.1, NZ_KK737548.1, 38601, 39326, +, 726, 20; cds-AF52_RS10495, NZ_KK737548.1, 39804, 40754, +, 951, 618; cds-WP _013815099.1-46, NZ_KK737548.1, 40806, 41774, -, 969, 3449; cds-AF52_RS10485, NZ_KK737548.1, 41809, 42504, +, 696, 4; cds-WP_032430005.1, NZ_KK737548.1, 42543, 43709, +, 1167, 16189; cds-WP_004895208.1, NZ_KK737548.1, 43759, 44478, +, 720, 1; cds-WP_004145102.1, NZ_KK737548.1, 44558, 45223, -, 666, 4; cds-WP_0041451001, NZ_KK737548.1, 45393, 46730, +, 1338, 18950; cds-WP_004151543.1, NZ_KK737548.1, 46776, 47342, -, 567, 5332; cds-WP_004145098.1, NZ_KK737548.1, 47379, 47954, -, 576, 2241; cds-WP_004211197.1, NZ_KK737548.1, 48264, 49223, -, 960, 21744; cds-WP _004181598.1, NZ_KK737548.1, 49198, 50376, -, 1179, 6366; cds-WP _004151539.1, NZ_KK737548.1, 50476, 50895, -, 420, 1013; cds-WP_004151538.1, NZ_KK737548.1, 51015, 52379, -, 1365, 2458; cds-WP_004150295.1, NZ_KK737548.1, 52574, 54220, -, 1647, 507683; cds-WP_004151536.1, NZ_KK737548.1, 54223, 54480, -, 258, 107480; cds-WP_004151535.1, NZ_KK737548.1, 54444, 54803, -, 360, 64588; cds-WP_000831330.1, NZ_KK737548.1, 54819, 54959, -, 141, 15915; cds-WP_004151534.1, NZ_KK737548.1, 55581, 56984, +, 1404, 94280; cds-WP _004145090.1, NZ_KK737548.1, 56989, 58089, +, 1101, 3943; cds-WP_004150293.1, NZ_KK737548.1, 58297, 59370, +, 1074, 14234; cds-WP _004173845.1, NZ_KK737548.1, 59399, 61813, +, 2415, 301071; cds-WP _050484285.1, NZ_KK737548.1, 62015, 62869, +, 855, 173; cds-WP _020806188.1-4, NZ_KK737548.1, 62970, 63950, -, 981, 6441; cds-AF52_RS25730, NZ_KK737548.1, 64019, 64090, +, 72, 286; cds-WP_021440944.1, NZ_KK737548.1, 64268, 65605, +, 1338, 89; cds-WP_004150284.1, NZ_KK737548.1, 65657, 66883, +, 1227, 206; cds-WP_004151524.1, NZ_KK737548.1, 66885, 67217, -, 333, 137; cds-WP_004151523.1, NZ_KK737548.1, 67532, 67945, +, 414, 259; cds-WP _004145074.1, NZ_KK737548.1, 68062, 68490, +, 429, 85; cds-WP _009308837.1, NZ_KK737548.1, 68627, 69451, +, 825, 3901; cds-WP_019705399.1, NZ_KK737548.1, 69553, 71214, +, 1662, 40; cds-WP_004151520.1, NZ_KK737548.1, 71206, 71928, -, 723, 465; cds-WP_002923307.1, NZ_KK737548.1, 72227, 73849, +, 1623, 628; cds-WP _002923306.1, NZ_KK737548.1, 73846, 75168, +, 1323, 4119; cds-AF52_RS24880, NZ_KK737548.1, 75270, 75617, +, 348, 22; cds-WP_002923304.1, NZ_KK737548.1, 75607, 75969, +, 363, 45; cds-WP _002923302.1, NZ_KK737548.1, 76044, 76466, +, 423, 1; cds-WP _020723563.1, NZ_KK737548.1, 76470, 77798, -, 1329, 8; cds-WP _004173855.1, NZ_KK737548.1, 77816, 79153, -, 1338, 5; cds-WP_032410116.1, NZ_KK737548.1, 79245, 79391, +, 147, 0; cds-WP_032430010.1, NZ_KK737548.1, 79379, 80302, +, 924, 1080; cds-WP_004181587.1, NZ_KK737548.1, 80462, 81646, -, 1185, 115; cds-WP_032430012.1, NZ_KK737548.1, 81822, 82655, -, 834, 3; cds-WP_002923294.1, NZ_KK737548.1, 82724, 83170, -, 447, 4; cds-WP _004145059.1, NZ_KK737548.1, 83261, 83380, -, 120, 0; cds-WP _004150272.1, NZ_KK737548.1, 83833, 84000, -, 168, 0; cds-WP_002923286.1, NZ_KK737548.1, 84013, 84108, +, 96, 96; cds-WP _004173858.1, NZ_KK737548.1, 84213, 85901, +, 1689, 56; cds-WP_002923283.1, NZ_KK737548.1, 85905, 86192, +, 288, 0; cds-WP _002923282.1, NZ_KK737548.1, 86343, 86936, +, 594, 6; cds-AF52_RS10285, NZ_KK737548.1, 86954, 88434, +, 1481, 8; cds-WP_004145056.1, NZ_KK737548.1, 88446, 89774, +, 1329, 0; cds-WP_002923280.1, NZ_KK737548.1, 89920, 91311, +, 1392, 0; cds-WP _004901545.1, NZ_KK737548.1, 91448, 91900, +, 453, 17; cds-WP_004173862.1, NZ_KK737548.1, 92029, 92352, -, 324, 6; cds-AF52_RS10260, NZ_KK737548.1, 92336, 92696, -, 361, 3; cds-WP_002923205.1, NZ_KK737548.1, 92908, 94101, +, 1194, 37490; cds-WP_021440939.1, NZ_KK737548.1, 94098, 94907, -, 810, 5492; cds-WP _021440938.1, NZ_KK737548.1, 94917, 96020, -, 1104, 5380; cds-WP_004145049.1, NZ_KK737548.1, 96162, 96881, +, 720, 0; cds-WP_021440937.1, NZ_KK737548.1, 97058, 98071, +, 1014, 0; cds-WP_004150258.1, NZ_KK737548.1, 98077, 99189, +, 1113, 0; cds-WP _004150257.1, NZ_KK737548.1, 99189, 100049, +, 861, 80; cds-WP _004145044.1, NZ_KK737548.1, 100052, 100849, +, 798, 1; cds-WP _032422633.1, NZ_KK737548.1, 101047, 102426, -, 1380, 6; cds-WP _002923158.1, NZ_KK737548.1, 102571, 102873, -, 303, 0; cds-WP _002923156.1, NZ_KK737548.1, 102971, 104293, -, 1323, 26; cds-WP _004145041.1, NZ_KK737548.1, 104305, 104619, -, 315, 1; cds-WP_002923154.1, NZ_KK737548.1, 104913, 105854, +, 942, 3; cds-WP _032426549.1, NZ_KK737548.1, 106066, 107436, +, 1371, 604; cds-WP _002923152.1, NZ_KK737548.1, 107450, 108712, +, 1263, 24145; cds-WP _032430013.1, NZ_KK737548.1, 108709, 109935, -, 1227, 21747; cds-WP _002923146.1, NZ_KK737548.1, 110063, 110803, +, 741, 1; cds-WP _032430014.1, NZ_KK737548.1, 110800, 111513, +, 714, 0; cds-WP_032428449.1, NZ_KK737548.1, 111514, 112599, +, 1086, 0; cds-WP _032428448.1, NZ_KK737548.1, 112602, 113465, +, 864, 1; cds-WP _004145033.1, NZ_KK737548.1, 113469, 113744, -, 276, 1027; cds-WP_004234160.1, NZ_KK737548.1, 113866, 114804, -, 939, 50141; cds-WP_004150245.1, NZ_KK737548.1, 114920, 116353, 1434, 5673; cds-WP_021440928.1, NZ_KK737548.1, 116378, 117280, -, 903, 1273; cds-WP_002923107.1, NZ_KK737548.1, 117443, 118606, -, 1164, 0; cds-WP _004889164.1, NZ _KK737548.1, 118652, 120016, -, 1365, 48; cds-WP_004151514.1, NZ_KK737548.1, 120020, 123127, -, 3108, 0; cds-WP _016529678.1, NZ _KK737548.1, 123127, 124251, -, 1125, 0; cds-WP _032412474.1, NZ_KK737548.1, 124458, 124580, +, 123, 1; cds-WP _032428455.1, NZ_KK737548.1, 124540, 124656, -, 117, 0; cds-WP_004173879.1, NZ_KK737548.1, 124631, 125047, +, 417, 5; cds-WP _004181564.1, NZ_KK737548.1, 125551, 126198, -, 648, 4546; cds-WP_032430016.1, NZ_KK737548.1, 126279, 127772, +, 1494, 1; cds-WP_002923020.1, NZ_KK737548.1, 127769, 128683, -, 915, 762; cds-WP_021440922.1, NZ_KK737548.1, 128779, 129405, +, 627, 0; cds-WP_021440921.1, NZ_KK737548.1, 129408, 129878, +, 471, 0; cds-WP _004145017.1, NZ_KK737548.1, 129899, 130435, -, 537, 0; cds-WP _004150236.1, NZ_KK737548.1, 130552, 131457, +, 906, 95; cds-WP_004173884.1, NZ_KK737548.1, 131586, 132395, +, 810, 1038; cds-WP_032430018.1, NZ_KK737548.1, 132420, 133274, +, 855, 4890; cds-WP_021440918.1, NZ_KK737548.1, 133264, 133824, -, 561, 3; cds-WP_004173886.1, NZ_KK737548.1, 133990, 134769, +, 780, 0; cds-WP _004145010.1, NZ_KK737548.1, 134791, 135744, +, 954, 2; cds-WP_004901462.1, NZ _KK737548.1, 135741, 136790, -, 1050, 0; cds-WP_004873113.1, NZ _KK737548.1, 137163, 137861, +, 699, 2; cds-WP_021440916.1, NZ_KK737548.1, 137912, 138202, -, 291, 0; cds-WP _002922964.1, NZ_KK737548.1, 138204, 139211, -, 1008, 0; cds-WP _002922963.1, NZ_KK737548.1, 139324, 139791, -, 468, 2; cds-WP _088296409.1, NZ_KK737548.1;NZ_KK737548.1, 140181;140442, 140442;141343, +;+, 1163, 506; cds-WP_108443077.1, NZ_KK737548.1, 141410, 141619, +, 210, 5549; cds-AF52_RS0124095, NZ_KK737548.1, 141929, 142276, -, 348, 1679; cds-AF52_RS0124105, NZ_KK737548.1, 142332, 143029, +, 698, 13238; cds-WP_021440909.1, NZ_KK737548.1, 143089, 144468, +, 1380, 23; cds-WP _004173889.1, NZ_KK737548.1, 144515, 145237, -, 723, 904; cds-AF52_RS0124110, NZ_KK737548.1, 145393, 147711, -, 2319, 1; cds-WP _002922962.1, NZ _KK737548.1, 147724, 149118, -, 1395, 0; cds-WP_032428443.1, NZ_KK737548.1, 149805, 151016, +, 1212, 47841; cds-WP_013815099.1-47, NZ_KK737548.1, 151813, 152781, -, 969, 4575; cds-WP_148673684.1, NZ_KK737548.1, 153177, 153419, -, 243, 0; cds-WP _013815099.1-48, NZ_KK737548.1, 153544, 154512, -, 969, 5237; cds-WP _050484287.1, NZ _KK737548.1, 154501, 155697, +, 1197, 0; cds-WP_002922944.1, NZ_KK737548.1, 155812, 157323, -, 1512, 667406; cds-WP_002922941.1, NZ_KK737548.1, 157345, 158196, -, 852, 52331; cds-WP_004150222.1, NZ_KK737548.1, 158639, 158884, +, 246, 5266; cds-WP_002922936.1, NZ_KK737548.1, 159036, 160025, +, 990, 45755; cds-WP_020803798.1, NZ_KK737548.1, 160188, 160958, +, 771, 1441; cds-WP_019724988.1, NZ_KK737548.1, 161023, 162327, +, 1305, 161; cds-WP_002922909.1, NZ_KK737548.1, 162420, 163187, -, 768, 62626; cds-WP_002922908.1, NZ_KK737548.1, 163297, 163899, -, 603, 121; cds-WP _021440903.1, NZ_KK737548.1, 164012, 164440, +, 429, 1; cds-WP _032430022.1, NZ_KK737548.1, 164441, 165187, -, 747, 5250; cds-WP _021440902.1, NZ_KK737548.1, 165338, 166348, -, 1011, 810; cds-WP _032428441.1, NZ_KK737548.1, 166397, 168478, -, 2082, 15838; cds-WP_004181538.1, NZ_KK737548.1, 168484, 169173, -, 690, 9448; cds-WP _004150214.1, NZ_KK737548.1, 169178, 171298, -, 2121, 331255; cds-WP_000135058.1, NZ_KK737548.1, 171317, 171592, -, 276, 78200; cds-WP _002922664.1, NZ_KK737548.1, 171647, 172270, -, 624, 44457; cds-WP_021440900.1, NZ_KK737548.1, 172529, 174205, +, 1677, 0; cds-WP_002922654.1, NZ_KK737548.1, 174211, 174828, -, 618, 11; cds-WP_021440899.1, NZ_KK737548.1, 175104, 176354, +, 1251, 7; cds-WP_004151500.1, NZ_KK737548.1, 176410, 177468, -, 1059, 7; cds-WP_023317368.1, NZ_KK737548.1, 177471, 178217, -, 747, 0; cds-WP_002922636.1, NZ_KK737548.1, 178400, 179263, -, 864, 6766; cds-AF52_RS27235, NZ_KK737548.1, 184808, 184969, -, 162, 272; cds-AF52_RS09865, NZ_KK737548.1, 185020, 185550, +, 531, 0; cds-AF52_RS27245, NZ_KK737548.1, 185584, 185730, +, 147, 219; cds-AF52_RS27250, NZ_KK737548.1, 189864, 189965, +, 102, 247; cds-AF52_RS09860, NZ_KK737548.1, 190018, 190773, +, 756, 6; cds-WP_004216995.1, NZ_KK737548.1, 190811, 190942, -, 132, 0; cds-WP_187079195.1, NZ_KK737548.1, 190941, 191795, +, 855, 1; cds-AF52_RS27260, NZ_KK737548.1, 191813, 191960, +, 148, 218; cds-AF52_RS09845, NZ_KK737548.1, 195939, 196123, +, 185, 520; cds-WP _032430029.1, NZ_KK737548.1, 196229, 197146, +, 918, 289; cds-AF52_RS09835, NZ_KK737548.1, 197178, 197753, +, 576, 14; cds-WP _013815099.1-49, NZ_KK737548.1, 197803, 198771, -, 969, 4507; cds-WP _032430030.1, NZ_KK737548.1, 198813, 199445, +, 633, 0; cds-WP _004181470.1, NZ_KK737548.1, 199673, 200161, +, 489, 62; cds-WP _009484082.1, NZ_KK737548.1, 200629, 202374, -, 1746, 190659; cds-WP _004150073.1, NZ_KK737548.1, 202498, 202818, +, 321, 0; cds-WP _021440835.1, NZ_KK737548.1, 202842, 203582, -, 741, 9; cds-WP _004150074.1, NZ_KK737548.1, 203579, 204649, -, 1071, 0; cds-WP_004145129.1, NZ_KK737548.1, 204651, 205496, -, 846, 18; cds-WP_014906831.1, NZ_KK737548.1, 205493, 206380, -, 888, 2470; cds-WP _004174008.1, NZ_KK737548.1, 206487, 207803, -, 1317, 45; cds-WP_025861226.1, NZ_KK737548.1, 208076, 208444, -, 369, 4; cds-WP_004174006.1, NZ_KK737548.1, 208441, 208662, -, 222, 105; cds-WP _004145133.1, NZ_KK737548.1, 208827, 209540, -, 714, 0; cds-WP _002920803.1, NZ_KK737548.1, 209542, 210309, -, 768, 0; cds-WP_004174005.1, NZ_KK737548.1, 210306, 211583, -, 1278, 0; cds-WP_002920807.1, NZ_KK737548.1, 211580, 212506, -, 927, 1; cds-WP_004150079.1, NZ_KK737548.1, 212568, 213677, -, 1110, 1; cds-WP_021312829.1, NZ_KK737548.1, 214101, 214484, +, 384, 3912; cds-WP _002920812.1, NZ_KK737548.1, 214753, 215856, -, 1104, 493; cds-WP _004150081.1, NZ_KK737548.1, 216114, 217358, -, 1245, 64; cds-WP _021440838.1, NZ_KK737548.1, 217506, 218771, -, 1266, 0; cds-WP_021440839.1, NZ_KK737548.1, 218890, 220413, +, 1524, 31427; cds-WP_032430034.1, NZ_KK737548.1, 220466, 221320, -, 855, 70848; cds-WP_004173994.1, NZ_KK737548.1, 221590, 222645, -, 1056, 1578; cds-WP_002920817.1, NZ_KK737548.1, 222638, 223306, -, 669, 25148; cds-WP_004173992.1, NZ_KK737548.1, 223309, 224832, -, 1524, 59532; cds-WP_002920820.1, NZ_KK737548.1, 225015, 225611, +, 597, 27; cds-WP_002920822.1, NZ_KK737548.1, 225601, 225876, +, 276, 69; cds-WP_004173989.1, NZ_KK737548.1, 225890, 226240, -, 351, 147; cds-WP _004173987.1, NZ_KK737548.1,
226391, 227017, +, 627, 102; cds-WP_004173985.1, NZ_KK737548.1, 227096, 229306, +, 2211, 1059; cds-WP_002920858.1, NZ_KK737548.1, 229410, 229655, -, 246, 14; cds-WP_002920860.1, NZ_KK737548.1, 229862, 230527, +, 666, 81487; cds-WP _002920861.1, NZ_KK737548.1, 230601, 231158, +, 558, 21215; cds-WP _004173981.1, NZ_KK737548.1, 231159, 232379, -, 1221, 42; cds-WP _004145147.1, NZ_KK737548.1, 232513, 233562, +, 1050, 35; cds-WP_002920863.1, NZ_KK737548.1, 233559, 233939, -, 381, 0; cds-WP _002920864.1, NZ_KK737548.1, 233939, 234277, -, 339, 0; cds-WP _004186016.1, NZ_KK737548.1, 234274, 235929, -, 1656, 14205; cds-WP _009308746.1, NZ_KK737548.1, 235929, 236831, -, 903, 11741; cds-WP_032430036.1, NZ_KK737548.1, 236828, 238813, -, 1986, 1243; cds-WP_002921035.1, NZ_KK737548.1, 238810, 239793, -, 984, 128; cds-WP_002921037.1, NZ_KK737548.1, 239795, 240934, -, 1140, 9; cds-WP _004181483.1, NZ_KK737548.1, 241331, 241837, -, 507, 52; cds-WP _032430038.1, NZ_KK737548.1, 241935, 242795, +, 861, 0; cds-WP_021440844.1, NZ_KK737548.1, 242934, 244502, +, 1569, 6; cds-WP_004150102.1, NZ_KK737548.1, 244502, 245446, +, 945, 1; cds-WP_004151422.1, NZ_KK737548.1, 245443, 246276, +, 834, 0; cds-WP _004173959.1, NZ_KK737548.1, 246276, 247040, +, 765, 0; cds-WP_009308750.1, NZ_KK737548.1, 247037, 247828, +, 792, 0; cds-WP_002921188.1, NZ_KK737548.1, 247816, 248214, +, 399, 0; cds-WP _002921192.1, NZ_KK737548.1, 248552, 249565, +, 1014, 0; cds-WP_004150109.1, NZ_KK737548.1, 249605, 250798, -, 1194, 32; cds-WP _002921200.1, NZ_KK737548.1, 251028, 252524, +, 1497, 7078; cds-WP _002921203.1, NZ_KK737548.1, 252580, 252915, -, 336, 30; cds-WP _004145166.1, NZ_KK737548.1, 253298, 253735, +, 438, 36715; cds-WP _002921264.1, NZ_KK737548.1, 253992, 255464, +, 1473, 46310; cds-WP_004173956.1, NZ_KK737548.1, 255592, 256344, -, 753, 148; cds-WP _004901172.1, NZ_KK737548.1, 256352, 258394, -, 2043, 8217; cds-WP_021465315.1, NZ_KK737548.1, 258577, 259848, -, 1272, 0; cds-WP _021440845.1, NZ_KK737548.1, 260042, 260884, +, 843, 980; cds-WP_002921271.1, NZ_KK737548.1, 260957, 262309, +, 1353, 24913; cds-WP_004173953.1, NZ_KK737548.1, 262363, 262986, -, 624, 64689; cds-WP_032430040.1, NZ_KK737548.1, 263113, 264261, +, 1149, 57; cds-WP _004150117.1, NZ_KK737548.1, 264501, 266153, +, 1653, 46979; cds-WP _032430042.1, NZ_KK737548.1, 266220, 266753, +, 534, 0; cds-WP_004211228.1, NZ_KK737548.1, 266788, 267546, -, 759, 1; cds-WP_004145179.1, NZ_KK737548.1, 267666, 268565, +, 900, 1130; cds-WP_002921337.1, NZ_KK737548.1, 268672, 269703, +, 1032, 25039; cds-WP _004150118.1, NZ_KK737548.1, 269975, 271297, +, 1323, 0; cds-WP_004901194.1, NZ_KK737548.1, 271335, 273386, -, 2052, 4; cds-WP _002921434.1, NZ_KK737548.1, 273581, 274510, +, 930, 126; cds-WP_032430046.1, NZ_KK737548.1, 274565, 276067, -, 1503, 69547; cds-WP _002921438.1, NZ_KK737548.1, 276287, 277573, -, 1287, 59384; cds-WP _072198875.1, NZ_KK737548.1, 277730, 279760, -, 2031, 6423; cds-WP _032430051.1, NZ_KK737548.1, 279979, 283458, -, 3480, 28; cds-WP _004173946.1, NZ_KK737548.1, 283440, 284549, -, 1110, 4; cds-WP_072001583.1, NZ_KK737548.1, 284553, 286898, -, 2346, 813; cds-WP _002921508.1, NZ_KK737548.1, 286909, 289527, -, 2619, 2; cds-WP _002921510.1, NZ_KK737548.1, 289524, 290255, -, 732, 0; cds-WP _002921513.1, NZ_KK737548.1, 290267, 290455, -, 189, 0; cds-WP _032430057.1, NZ_KK737548.1, 290624, 292177, +, 1554, 86; cds-WP_002921518.1, NZ_KK737548.1, 292177, 292374, +, 198, 2; cds-WP_004188236.1, NZ_KK737548.1, 292371, 294050, +, 1680, 2; cds-WP_032430059.1, NZ_KK737548.1, 294158, 295159, -, 1002, 0; cds-WP _002921529.1, NZ_KK737548.1, 295171, 295647, -, 477, 49; cds-WP_032430061.1, NZ_KK737548.1, 295647, 299699, -, 4053, 1; cds-WP_004151435.1, NZ_KK737548.1, 299696, 302131, -, 2436, 1; cds-WP _004173941.1, NZ_KK737548.1, 302128, 304239, -, 2112, 70; cds-WP _004173940.1, NZ_KK737548.1, 304259, 305062, -, 804, 2430; cds-WP _004186070.1, NZ_KK737548.1, 305053, 305592, -, 540, 1731; cds-WP _004145206.1, NZ_KK737548.1, 305910, 306089, -, 180, 57; cds-WP_021440859.1, NZ_KK737548.1, 306108, 307121, -, 1014, 0; cds-WP_021440860.1, NZ_KK737548.1, 307118, 308101, -, 984, 0; cds-WP_004145207.1, NZ_KK737548.1, 308112, 309014, -, 903, 0; cds-WP_002921786.1, NZ_KK737548.1, 309024, 310043, -, 1020, 0; cds-WP_050484288.1, NZ_KK737548.1, 310178, 311779, -, 1602, 0; cds-WP_013815099.1-50, NZ_KK737548.1, 311759, 312727, +, 969, 6399; cds-WP_004214574.1, NZ_KK737548.1, 313931, 315325, -, 1395, 0; cds-WP_004152427.1, NZ_KK737548.1, 315517, 317190, -, 1674, 87878; cds-WP _002921907.1, NZ_KK737548.1, 317433, 318638, -, 1206, 227; cds-WP _020803959.1, NZ_KK737548.1, 318880, 319461, -, 582, 2; cds-WP _002921914.1, NZ_KK737548.1, 319667, 320248, +, 582, 34178; cds-WP _015959126.1, NZ_KK737548.1, 320226, 320666, +, 441, 1493; cds-WP _032428435.1, NZ_KK737548.1, 320635, 322965, -, 2331, 22683; cds-WP_002921917.1, NZ_KK737548.1, 323133, 323795, +, 663, 61; cds-WP _002921918.1, NZ_KK737548.1, 323899, 324915, +, 1017, 57523; cds-WP_002921919.1, NZ_KK737548.1, 325027, 325776, +, 750, 5797; cds-WP _002921921.1, NZ_KK737548.1, 325780, 326715, +, 936, 38; cds-WP_002921927.1, NZ_KK737548.1, 326820, 328100, +, 1281, 7756; cds-WP_021440865.1, NZ_KK737548.1, 328125, 329096, +, 972, 1567; cds-WP_002921929.1, NZ_KK737548.1, 329205, 329915, -, 711, 4202; cds-WP_021440866.1, NZ_KK737548.1, 330356, 331711, +, 1356, 0; cds-WP _013815099.1-51, NZ_KK737548.1, 331921, 332889, -, 969, 4442; cds-AF52_RS26455, NZ_KK737548.1, 332922, 333029, -, 108, 0; cds-WP_002922127.1, NZ_KK737548.1, 333330, 335399, -, 2070, 99597; cds-WP _002922128.1, NZ_KK737548.1, 335409, 336320, -, 912, 47159; cds-WP_002922129.1, NZ_KK737548.1, 336447, 336746, -, 300, 0; cds-WP _004145225.1, NZ_KK737548.1, 336926, 337921, +, 996, 787; cds-WP_004188180.1, NZ_KK737548.1, 337948, 338394, -, 447, 967; cds-WP _002922133.1, NZ_KK737548.1, 338426, 338797, -, 372, 0; cds-WP _004150156.1, NZ_KK737548.1, 338966, 340420, -, 1455, 13; cds-WP_021440868.1, NZ_KK737548.1, 340513, 341835, -, 1323, 4861; cds-WP_014906789.1, NZ_KK737548.1, 342270, 343325, +, 1056, 6; cds-WP _002922346.1, NZ_KK737548.1, 343390, 344931, +, 1542, 32087; cds-WP_002922349.1, NZ_KK737548.1, 344909, 346090, +, 1182, 4; cds-WP_002922351.1, NZ_KK737548.1, 346323, 347501, +, 1179, 2721; cds-WP_004188166.1, NZ_KK737548.1, 347540, 348358, -, 819, 8; cds-WP _004173926.1, NZ_KK737548.1, 348673, 350706, +, 2034, 24344; cds-WP _002922359.1, NZ_KK737548.1, 350875, 352131, +, 1257, 17325; cds-WP _021440872.1, NZ_KK737548.1, 352174, 352659, -, 486, 1; cds-WP _021440873.1, NZ_KK737548.1, 352769, 353596, -, 828, 475; cds-WP_021440874.1, NZ_KK737548.1, 353815, 354813, +, 999, 11; cds-WP _021440875.1, NZ_KK737548.1, 354825, 355289, +, 465, 0; cds-WP_004150167.1, NZ_KK737548.1, 355314, 356243, +, 930, 8673; cds-WP _032430066.1, NZ_KK737548.1, 356280, 357599, +, 1320, 10858; cds-WP_021440877.1, NZ_KK737548.1, 357678, 359183, +, 1506, 1; cds-WP_004889285.1, NZ_KK737548.1, 359180, 359842, +, 663, 0; cds-WP_004889283.1, NZ_KK737548.1, 359835, 360695, +, 861, 0; cds-WP_021440878.1, NZ_KK737548.1, 360689, 361387, +, 699, 8; cds-WP_032430069.1, NZ_KK737548.1, 361445, 363286, -, 1842, 11; cds-WP _021440880.1, NZ_KK737548.1, 363283, 364671, -, 1389, 26820; cds-WP_004145238.1, NZ_KK737548.1, 364777, 365385, -, 609, 3706; cds-AF52_RS26460, NZ_KK737548.1, 365492, 365822, -, 331, 0; cds-WP_002922397.1, NZ_KK737548.1, 366352, 368259, +, 1908, 17445; cds-WP _019705056.1, NZ_KK737548.1, 368360, 369508, +, 1149, 50; cds-WP_002922401.1, NZ_KK737548.1, 369508, 370104, +, 597, 194; cds-WP_002922402.1, NZ_KK737548.1, 370093, 370317, -, 225, 0; cds-WP_002922410.1, NZ_KK737548.1, 370590, 370952, +, 363, 2865; cds-WP _002922413.1, NZ_KK737548.1, 371238, 372893, +, 1656, 961977; cds-WP _002922415.1, NZ_KK737548.1, 372890, 373663, +, 774, 182941; cds-WP _002922420.1, NZ_KK737548.1, 373663, 374847, +, 1185, 114488; cds-WP _002922423.1, NZ_KK737548.1, 374940, 375413, +, 474, 144831; cds-WP_004164645.1, NZ_KK737548.1, 375490, 375757, +, 268, 3576; cds-WP _032430073.1, NZ_KK737548.1, 376056, 376773, +, 718, 6385; cds-WP_004185922.1, NZ_KK737548.1, 376896, 377726, -, 831, 76711; cds-WP _002920136.1, NZ_KK737548.1, 377949, 378167, +, 219, 5; cds-WP _002920140.1, NZ_KK737548.1, 378226, 378813, -, 588, 35254; cds-WP _002920143.1, NZ_KK737548.1, 378907, 379107, -, 201, 64; cds-WP _004150031.1, NZ_KK737548.1, 379119, 380924, -, 1806, 5191; cds-WP _004151401.1, NZ_KK737548.1, 380911, 381474, -, 564, 435; cds-WP _032430077.1, NZ_KK737548.1, 381648, 383552, +, 1905, 81313; cds-WP_072145335.1, NZ_KK737548.1, 383780, 384778, +, 999, 1032; cds-WP_002920151.1, NZ_KK737548.1, 384775, 384993, +, 219, 10; cds-WP _021440811.1, NZ_KK737548.1, 385030, 385899, +, 870, 36261; cds-WP _002920158.1, NZ_KK737548.1, 385954, 386358, -, 405, 6887; cds-WP 000242758.1, NZ_KK737548.1, 386665, 387297, +, 633, 305413; cds-WP _004213494.1, NZ_KK737548.1, 387349, 389427, +, 2079, 16271; cds-WP _021440813.1, NZ_KK737548.1, 389417, 390637, -, 1221, 275; cds-WP_021440814.1, NZ_KK737548.1, 390728, 391291, -, 564, 15; cds-WP _004181446.1, NZ_KK737548.1, 391324, 391926, -, 603, 14; cds-WP _002920231.1, NZ_KK737548.1, 391916, 392083, -, 168, 88; cds-WP _002920238.1, NZ_KK737548.1, 392190, 392759, -, 570, 12570; cds-WP_002920240.1, NZ_KK737548.1, 393036, 394223, +, 1188, 1196; cds-WP_004174045.1, NZ_KK737548.1, 394220, 395542, -, 1323, 12752; cds-WP_002920243.1, NZ_KK737548.1, 395781, 398324, +, 2544, 36; cds-WP_002920249.1, NZ_KK737548.1, 398321, 398647, +, 327, 0; cds-WP_002920252.1, NZ_KK737548.1, 398801, 400174, +, 1374, 743; cds-WP_002920253.1, NZ_KK737548.1, 400247, 401251, -, 1005, 204696; cds-WP_004188345.1, NZ_KK737548.1, 401244, 402005, -, 762, 20155; cds-WP_002920259.1, NZ_KK737548.1, 401998, 402675, -, 678, 29998; cds-WP_002920260.1, NZ_KK737548.1, 402693, 403520, -, 828,120113; cds-WP_021440816.1, NZ_KK737548.1, 403599, 404885, -, 1287, 50822; cds-WP_004150047.1, NZ_KK737548.1, 404970, 406064, -, 1095, 80645; cds-WP_002920267.1, NZ_KK737548.1, 406120, 406641, -, 522, 37616; cds-WP _021440817.1, NZ_KK737548.1, 406912, 408156, -, 1245, 28; cds-WP _002920279.1, NZ_KK737548.1, 408089, 408472, -, 384, 0; cds-WP_004174029.1, NZ_KK737548.1, 408462, 408890, -, 429, 0; cds-WP_032428423.1, NZ_KK737548.1, 408880, 409413, -, 534, 0; cds-WP_004174027.1, NZ_KK737548.1, 409397, 410197, -, 801, 0; cds-WP_004185951.1, NZ_KK737548.1, 410316, 412874, +, 2559, 60576; cds-WP _032430083.1, NZ_KK737548.1, 412928, 413488, -, 561, 6649; cds-WP_032430086.1, NZ_KK737548.1, 413809, 415938, +, 2130, 722; cds-WP_004181456.1, NZ_KK737548.1, 415999, 416682, +, 684, 3656; cds-WP_002920326.1, NZ_KK737548.1, 416679, 417080, +, 402, 2640; cds-WP_004188324.1, NZ_KK737548.1, 417099, 417983, +, 885, 652; cds-WP _032430089.1, NZ_KK737548.1, 418344, 419966, +, 1623, 189131; cds-WP_002920333.1, NZ_KK737548.1, 420044, 421399, -, 1356, 50; cds-WP_001157751.1, NZ_KK737548.1, 421396, 422115, -, 720, 1838; cds-WP _002920503.1, NZ_KK737548.1, 422343, 422816, +, 474, 4; cds-WP _004150056.1, NZ_KK737548.1, 422917, 425247, +, 2331, 203816; cds-WP_013815099.1-52, NZ_KK737548.1, 425442, 426410, -, 969, 2658; cds-WP_002920506.1, NZ_KK737548.1, 426726, 426953, +, 228,6492; cds-WP_014906839.1, NZ_KK737548.1, 426982, 429300, +, 2319, 5295; cds-WP_002920509.1, NZ_KK737548.1, 429310, 429549, +, 240, 0; cds-WP _002920510.1, NZ_KK737548.1, 429638, 429907, -, 270, 6; cds-WP _032430095.1, NZ_KK737548.1, 430028, 430801, -, 774,136; cds-WP_004185961.1, NZ_KK737548.1, 430840, 431514, +, 675, 11; cds-WP_002920540.1, NZ_KK737548.1, 431573, 432148, +, 576, 2086; cds-WP_002920542.1, NZ_KK737548.1, 432485, 433801, +, 1317, 10; cds-WP _004185965.1, NZ_KK737548.1, 433904, 435997, -, 2094, 15189; cds-WP _004151407.1, NZ_KK737548.1, 436008, 438398, -, 2391, 223585; cds-WP _002920545.1, NZ_KK737548.1, 439023, 441728, +, 2706, 153020; cds-WP _002920548.1, NZ_KK737548.1, 441744, 442502, -, 759, 13848; cds-WP_032428425.1, NZ_KK737548.1, 442552, 443382, -, 831, 30867; cds-WP_002920552.1, NZ_KK737548.1, 443433, 443762, -, 330, 5090; cds-WP_002920554.1, NZ_KK737548.1, 443967, 445475, +, 1509, 69394; cds-WP_004174017.1, NZ_KK737548.1, 445538, 445837, -, 300, 0; cds-WP _016529833.1, NZ_KK737548.1, 445837, 446184, -, 348, 839; cds-WP_002920561.1, NZ_KK737548.1, 446360, 448807, -, 2448, 6228; cds-WP _002920564.1, NZ_KK737548.1, 448825, 450258, -, 1434, 6701; cds-WP_002920566.1, NZ_KK737548.1, 450258, 451553, -, 1296, 1881; cds-WP_023342583.1, NZ_KK737548.1, 451634, 453610, -, 1977, 33; cds-WP_004150069.1, NZ_KK737548.1,
453607, 455793, -, 2187, 60111; cds-WP_002920570.1, NZ_KK737548.1, 456031, 457137, -, 1107, 360; cds-WP_004151411.1, NZ_KK737548.1, 457241, 457603, +, 363, 0; cds-WP_002920773.1, NZ_KK737548.1, 457600, 458940, -, 1341, 9610; cds-WP_002920775.1, NZ_KK737548.1, 458940, 459470, -, 531, 666; cds-WP_002920777.1, NZ_KK737548.1, 459651, 460646, -, 996, 27440; cds-WP_002920779.1, NZ_KK737548.1, 460760, 461455, -, 696, 173; cds-WP_021440831.1, NZ_KK737548.1, 461577, 462614, -, 1038, 331; cds-WP_013815099.1-53, NZ_KK737548.1, 462956, 463924, -, 969, 3725; cds-AF52_RS08695, NZ_KK737548.1, 463958, 464491, +, 534, 53; cds-WP_002922602.1, NZ_KK737548.1, 464588, 465304, +, 717, 305; cds-WP _004173904.1, NZ_KK737548.1, 465415, 466056, +, 642, 52331; cds-WP _004173905.1, NZ_KK737548.1, 466092, 467108, -, 1017, 17256; cds-WP_002922598.1, NZ_KK737548.1, 467284, 467880, -, 597, 3209; cds-WP_004145270.1, NZ_KK737548.1, 468005, 468463, -, 459, 19879; cds-WP_004188117.1, NZ_KK737548.1, 468441, 469655, -, 1215, 17283; cds-WP_002922589.1, NZ_KK737548.1, 469828, 470493, +, 666, 41358; cds-WP _000091955.1, NZ_KK737548.1, 470710, 470946, +, 237, 154350; cds-WP_002922510.1, NZ_KK737548.1, 470967, 471134, +, 168, 52214; cds-WP _004173907.1, NZ_KK737548.1, 471270, 472079, +, 810, 76; cds-WP _002922501.1, NZ_KK737548.1, 472268, 472747, -, 480, 41; cds-WP_004145264.1, NZ_KK737548.1, 472744, 473520, -, 777, 26567; cds-WP_004145263.1, NZ_KK737548.1, 473520, 474794, -, 1275, 1194; cds-WP _002922498.1, NZ_KK737548.1, 474886, 475875, -, 990, 7099; cds-WP _032430101.1, NZ_KK737548.1, 475905, 476999, -, 1095, 99; cds-WP _009308781.1, NZ_KK737548.1, 477002, 478129, -, 1128, 12695; cds-WP_021440889.1, NZ_KK737548.1, 478126, 479202, -, 1077, 20421; cds-WP _021440888.1, NZ_KK737548.1, 479314, 480276, +, 963, 44723; cds-WP _032428440.1, NZ_KK737548.1, 480295, 481374, +, 1080, 9733; cds-WP _032430104.1, NZ_KK737548.1, 481418, 482587, -, 1170, 11665; cds-WP_002922468.1, NZ_KK737548.1, 482565, 483473, -, 909, 18257; cds-WP_004186128.1, NZ_KK737548.1, 483473, 484444, -, 972, 2469; cds-WP_002922465.1, NZ_KK737548.1, 484448, 485506, -, 1059, 3060; cds-WP _002922463.1, NZ_KK737548.1, 485516, 486448, -, 933, 78343; cds-WP_002922462.1, NZ_KK737548.1, 486662, 487855, +, 1194, 59787; cds-WP_002922461.1, NZ_KK737548.1, 487868, 488893, +, 1026, 30341; cds-WP_032428438.1, NZ_KK737548.1, 489062, 489850, +, 789, 24301; cds-WP_002922459.1, NZ_KK737548.1, 489856, 490803, -, 948, 2245; cds-WP _032428437.1, NZ_KK737548.1, 490807, 492078, -, 1272, 40144; cds-WP_004152046.1, NZ_KK737548.1, 492088, 493632, -, 1545, 50; cds-WP_002922436.1, NZ_KK737548.1, 493878, 494309, +, 432, 61157; cds-WP_002922429.1, NZ_KK737548.1, 494416, 494667, +, 252, 42171; cds-WP _002922428.1, NZ_KK737548.1, 494728, 495195, +, 468, 90834; cds-WP _002922427.1, NZ_KK737548.1, 495195, 496214, +, 1020, 108248; cds-WP_002922426.1, NZ_KK737548.1, 496288, 497109, +, 822, 3788; cds-AF52_RS27300, NZ_KK737548.1, 497212, 497488, -, 277, 2228; cds-WP_032430113.1, NZ_KK737548.1, 498141, 498462, -, 322, 825; cds-WP_002920130.1, NZ_KK737548.1, 498606, 499328, +, 723, 6529; cds-WP_002920128.1, NZ_KK737548.1, 499328, 499714, +, 387, 1; cds-WP_004889490.1, NZ_KK737548.1, 499714, 500073, +, 360, 127; cds-WP _002920119.1, NZ_KK737548.1, 500081, 500368, +, 288, 370; cds-WP_002920115.1, NZ_KK737548.1, 500493, 500867, +, 375, 558210; cds-WP_002920113.1, NZ_KK737548.1, 500964, 501434, +, 471, 1256241; cds-WP_002920103.1, NZ_KK737548.1, 501531, 503645, +, 2115, 5379147; cds-WP _004174069.1, NZ_KK737548.1, 503715, 504899, +, 1185, 5313536; cds-WP_004152415.1, NZ_KK737548.1, 505080, 505274, +, 195, 28800; cds-WP _002919971.1, NZ_KK737548.1, 505348, 505824, +, 477, 61616; cds-WP_020801891.1, NZ_KK737548.1, 505808, 506293, -, 486, 34; cds-WP _001181005.1, NZ_KK737548.1, 506677, 506988, +, 312, 484272; cds-WP_002919796.1, NZ_KK737548.1, 507021, 507650, +, 630, 1110399; cds-WP _002919794.1, NZ_KK737548.1, 507661, 508266, +, 606, 1813373; cds-WP _000617546.1, NZ_KK737548.1, 508263, 508565, +, 303, 742840; cds-WP _002919786.1, NZ_KK737548.1, 508583, 509404, +, 822, 1811558; cds-WP _001138115.1, NZ_KK737548.1, 509421, 509699, +, 279, 1378924; cds-WP _002919773.1, NZ_KK737548.1, 509714, 510046, +, 333, 1775303; cds-WP _002919766.1, NZ_KK737548.1, 510064, 510762, +, 699, 1917106; cds-WP_002919759.1, NZ_KK737548.1, 510775, 511185, +, 411, 1138021; cds-WP_002919754.1, NZ_KK737548.1, 511185, 511376, +, 192, 470677; cds-WP _002919751.1, NZ_KK737548.1, 511376, 511630, +, 255, 350824; cds-WP_002919748.1, NZ_KK737548.1, 511796, 512167, +, 372, 240800; cds-WP_000729185.1, NZ_KK737548.1, 512178, 512492, +, 315, 556271; cds-WP 001096200.1, NZ_KK737548.1, 512507, 513046, +, 540, 1582314; cds-WP_002919667.1, NZ_KK737548.1, 513061, 513366, +, 306, 1236396; cds-WP_002919665.1, NZ_KK737548.1, 513400, 513792, +, 393, 704910; cds-WP_002919662.1, NZ_KK737548.1, 513805, 514338, +, 534, 1098814; cds-WP _000358960.1, NZ_KK737548.1, 514348, 514701, +, 354, 975894; cds-WP_002919545.1, NZ_KK737548.1, 514716, 515219, +, 504, 747206; cds-WP_001140434.1, NZ_KK737548.1, 515223, 515402, +, 180, 356655; cds-WP _002919516.1, NZ_KK737548.1, 515406, 515840, +, 435, 1660488; cds-WP _002919515.1, NZ_KK737548.1, 515848, 517179, +, 1332, 2541458; cds-WP_000868187.1, NZ_KK737548.1, 517213, 517329, +, 117, 196565; cds-WP_002919259.1, NZ_KK737548.1, 517476, 517832, +, 357, 894926; cds-WP _002919257.1, NZ_KK737548.1, 517849, 518238, +, 390, 1399975; cds-WP_002919224.1, NZ_KK737548.1, 518272, 518892, +, 621, 1683053; cds-WP _002919219.1, NZ_KK737548.1, 518918, 519907, +, 990, 2679755; cds-WP_002919206.1, NZ_KK737548.1, 519948, 520334, +, 387, 535665; cds-WP_002919204.1, NZ_KK737548.1, 520443, 520811, +, 369, 2869; cds-WP_004185894.1, NZ_KK737548.1, 520814, 521239, +, 426, 5912; cds-WP_004145324.1, NZ_KK737548.1, 521297, 521575, +, 279, 2; cds-WP_004145325.1, NZ_KK737548.1, 521511, 521924, -, 414, 6617; cds-WP _032430127.1, NZ_KK737548.1, 522065, 523441, -, 1377, 11910; cds-WP _004212524.1, NZ_KK737548.1, 523455, 524750, -, 1296, 43275; cds-WP_032430129.1, NZ_KK737548.1, 524803, 525750, -, 948, 39804; cds-WP_004889527.1, NZ_KK737548.1, 525765, 526274, -, 510, 6761; cds-WP _032428422.1, NZ_KK737548.1, 526403, 527527, +, 1125, 7726; cds-WP _002919137.1, NZ_KK737548.1, 527499, 527972, +, 474, 39215; cds-WP_004145330.1, NZ_KK737548.1, 527999, 528541, +, 543, 46869; cds-WP _021440795.1, NZ_KK737548.1, 528546, 529118, +, 573, 16256; cds-WP_002919126.1, NZ_KK737548.1, 529122, 529940, +, 819, 2980; cds-WP_002919125.1, NZ_KK737548.1, 529937, 530194, +, 258, 36; cds-AF52_RS08260, NZ_KK737548.1, 530170, 530502, -, 333, 4; cds-AF52_RS08255, NZ_KK737548.1, 530559, 530709, -, 151, 300; cds-AF52_RS08250, NZ_KK737548.1, 538177, 538410, -, 234, 19; cds-WP_002919103.1, NZ_KK737548.1, 546983, 547204, -, 222, 0; cds-WP _032430131.1, NZ_KK737548.1, 547497, 550607, -, 3111, 235; cds-WP _002919101.1, NZ_KK737548.1, 550620, 551759, -, 1140, 17; cds-WP_021440791.1, NZ_KK737548.1, 552138, 552791, +, 654, 0; cds-WP_000462905.1, NZ_KK737548.1, 552876, 553172, -, 297, 27073; cds-WP _004144972.1, NZ_KK737548.1, 553197, 554162, -, 966, 95131; cds-WP _004144971.1, NZ_KK737548.1, 554520, 555401, -, 882, 14841; cds-WP_004188394.1, NZ_KK737548.1, 555413, 556864, -, 1452, 13239; cds-WP _002918740.1, NZ_KK737548.1, 556854, 557096, -, 243, 440; cds-WP _002918738.1, NZ_KK737548.1, 557207, 558556, -, 1350, 112325; cds-WP_002918736.1, NZ_KK737548.1, 558567, 559034, -, 468, 63644; cds-WP_019725248.1, NZ_KK737548.1, 559057, 559509, -, 453, 275700; cds-WP_002918689.1, NZ_KK737548.1, 559733, 560341, -, 609, 44; cds-WP_004211677.1, NZ_KK737548.1, 560341, 561342, -, 1002, 2; cds-WP _004211674.1, NZ_KK737548.1, 561572, 561763, +, 192, 6; cds-WP _002918686.1, NZ_KK737548.1, 561843, 563783, +, 1941, 4615; cds-WP_002918653.1, NZ_KK737548.1, 564089, 565132, +, 1044, 104680; cds-WP _004149974.1, NZ_KK737548.1, 565203, 566195, +, 993, 19077; cds-WP _002918648.1, NZ_KK737548.1, 566195, 566683, +, 489, 20341; cds-WP _032430132.1, NZ_KK737548.1, 566691, 567272, +, 582, 2953; cds-WP _002918644.1, NZ_KK737548.1, 567275, 568744, +, 1470, 80793; cds-WP_032417624.1, NZ_KK737548.1, 568782, 572579, +, 3798, 43939; cds-WP _004144958.1, NZ_KK737548.1, 572668, 574113, +, 1446, 80694; cds-WP_002918641.1, NZ_KK737548.1, 574149, 575078, -, 930, 87; cds-WP_002918640.1, NZ_KK737548.1, 575210, 575413, +, 204, 13; cds-WP_002918639.1, NZ_KK737548.1, 575421, 576353, +, 933, 36972; cds-WP _002918632.1, NZ_KK737548.1, 576359, 578326, +, 1968, 2; cds-WP_002918629.1, NZ_KK737548.1, 578406, 578681, +, 276, 212; cds-WP_002918627.1, NZ_KK737548.1, 578732, 578998, -, 267, 4; cds-WP _032428421.1, NZ_KK737548.1, 579097, 579360, -, 264, 13; cds-WP _002918625.1, NZ_KK737548.1, 579735, 580205, -, 471, 6724; cds-WP_002918570.1, NZ_KK737548.1, 580620, 581558, +, 939, 138149; cds-WP_002918568.1, NZ_KK737548.1, 581695, 582753, -, 1059, 9525; cds-WP_032430134.1, NZ_KK737548.1, 582841, 584208, -, 1368, 36; cds-WP _002918565.1, NZ_KK737548.1, 584382, 584780, -, 399, 20733; cds-WP_004900926.1, NZ_KK737548.1, 584971, 586098, +, 1128, 136975; cds-WP _002918559.1, NZ_KK737548.1, 586364, 586792, +, 429, 462292; cds-WP_000829818.1, NZ_KK737548.1, 586808, 587200, +, 393, 829996; cds-WP _002918467.1, NZ_KK737548.1, 587512, 588150, +, 639, 95770; cds-WP _002918465.1, NZ_KK737548.1, 588154, 588648, +, 495, 16819; cds-WP _004185859.1, NZ_KK737548.1, 588773, 589477, +, 705, 187; cds-WP_002918461.1, NZ_KK737548.1, 589532, 590950, -, 1419, 36736; cds-WP_004144939.1, NZ_KK737548.1, 590960, 595420, -4461, 183936; cds-WP _002918455.1, NZ_KK737548.1, 596094, 597026, +, 933, 6; cds-AF52_RS08010, NZ_KK737548.1, 597078, 597653, -, 576, 9; cds-AF52_RS08005, NZ_KK737548.1, 597707, 597899, -, 193, 289; cds-WP _032430135.1, NZ_KK737548.1, 600002, 601147, -, 1146, 38586; cds-WP_002917952.1, NZ_KK737548.1, 601212, 602102, -891, 93386; cds-WP _002917954.1, NZ_KK737548.1, 602129, 602899, -, 771, 29803; cds-WP _020802049.1, NZ_KK737548.1, 602926, 604257, -, 1332, 59840; cds-WP_002917960.1, NZ_KK737548.1, 604650, 606221, +, 1572, 47601; cds-WP_004144878.1, NZ_KK737548.1, 606434, 607297, -, 864, 5115; cds-WP _032430136.1, NZ_KK737548.1, 607361, 609469, +, 2109, 18603; cds-WP_002918206.1, NZ_KK737548.1, 609427, 609813, +, 387, 401; cds-WP _002918211.1, NZ_KK737548.1, 609839, 610429, +, 591, 1264; cds-WP_002918214.1, NZ_KK737548.1, 610439, 611014, +, 576, 2662; cds-WP_004149921.1, NZ_KK737548.1, 611136, 612176, -, 1041, 4292; cds-WP _002918218.1, NZ_KK737548.1, 612252, 612899, -, 648, 0; cds-WP _002918221.1, NZ_KK737548.1, 613028, 613564, +, 537, 0; cds-WP _002918223.1, NZ_KK737548.1, 613526, 613969, -, 444, 5521; cds-WP_002918226.1, NZ_KK737548.1, 614019, 614309, +, 291, 3162; cds-WP _002918229.1, NZ_KK737548.1, 614296, 614799, -, 504, 6; cds-WP _002918231.1, NZ_KK737548.1, 614793, 615317, -, 525, 101; cds-WP_002918232.1, NZ_KK737548.1, 615539, 616534, +, 996, 0; cds-WP_020802035.1, NZ_KK737548.1, 616540, 617418, +, 879, 682; cds-WP_020802054.1, NZ_KK737548.1, 617562, 618569, +, 1008, 13785; cds-WP _002918235.1, NZ_KK737548.1, 618614, 619858, -, 1245, 10; cds-WP _004150943.1, NZ_KK737548.1, 620003, 621934, -, 1932, 43996; cds-WP_085903200.1, NZ_KK737548.1, 621927, 622007, -, 81, 43774; cds-WP _002918241.1, NZ_KK737548.1, 622112, 622996, -, 885, 157847; cds-WP_004150944.1, NZ_KK737548.1, 623105, 625240, -, 2136, 554174; cds-WP _002918244.1, NZ_KK737548.1, 625483, 625752, -, 270, 44638; cds-WP _023280600.1, NZ_KK737548.1, 625901, 626845, -, 945, 37001; cds-WP_002918248.1, NZ_KK737548.1, 626845, 627246, -, 402, 8870; cds-WP_002918250.1, NZ_KK737548.1, 627349, 630039, -2691, 688553; cds-WP_002918252.1, NZ_KK737548.1, 630064, 631551, -, 1488, 284367; cds-WP_002918364.1, NZ_KK737548.1, 631579, 632031, -, 453, 80046; cds-WP_004144895.1, NZ_KK737548.1, 632624, 633967, +, 1344, 33330; cds-WP_013815099.1-54, NZ_KK737548.1, 634121, 635089, -, 969, 7690; cds-AF52_RS0124215, NZ_KK737548.1, 635123, 635506, +, 384, 1831; cds-WP_004174150.1, NZ_KK737548.1, 635644, 636273, +, 630, 1; cds-WP_002918367.1, NZ_KK737548.1, 636616, 637167, +, 552, 0; cds-WP_014906872.1, NZ_KK737548.1, 637209, 637457, -, 249, 0; cds-WP_002918369.1, NZ_KK737548.1, 637851, 638180, -, 330, 2597; cds-WP _002918370.1, NZ_KK737548.1, 638410, 639747, -, 1338, 54044; cds-WP_002918371.1, NZ_KK737548.1, 639740, 640588, -, 849, 3607; cds-WP_002918372.1, NZ_KK737548.1, 640682, 642616,
-, 1935, 667533; cds-WP _002918373.1, NZ_KK737548.1, 642716, 643345, -, 630, 20446; cds-WP_002918374.1, NZ_KK737548.1, 643469, 643762, +, 294, 24299; cds-WP_002918375.1, NZ_KK737548.1, 643915, 644391, -, 477, 40527; cds-WP_004149937.1, NZ_KK737548.1, 644627, 646060, +, 1434, 11082; cds-WP _002918377.1, NZ_KK737548.1, 646112, 647290, -, 1179, 256031; cds-WP_004149938.1, NZ_KK737548.1, 647306, 648271, -, 966, 24951; cds-WP _002434222.1, NZ_KK737548.1, 648365, 648622, -, 258, 159445; cds-WP _002918379.1, NZ_KK737548.1, 648643, 648954, -, 312, 318725; cds-WP _004144907.1, NZ_KK737548.1, 649215, 650186, +, 972, 44921; cds-WP_002918381.1, NZ_KK737548.1, 650383, 650655, +, 273, 15; cds-WP_002918382.1, NZ_KK737548.1, 650711, 651970, -, 1260, 35347; cds-WP _004144910.1, NZ_KK737548.1, 652024, 652278, -, 255, 10437; cds-WP _004174142.1, NZ_KK737548.1, 652415, 652705, -, 291, 6706; cds-WP _032430139.1, NZ_KK737548.1, 652705, 653340, -, 636, 14426; cds-WP_002918387.1, NZ_KK737548.1, 653359, 653910, -, 552, 2039; cds-WP_004150949.1, NZ_KK737548.1, 653915, 654697, -, 783, 28787; cds-WP_004150950.1, NZ_KK737548.1, 654705, 655517, -, 813, 121791; cds-WP_004149949.1, NZ_KK737548.1, 655727, 656704, +, 978, 31; cds-WP _002918399.1, NZ_KK737548.1, 656719, 657705, +, 987, 2327; cds-WP_002918405.1, NZ_KK737548.1, 657720, 658286, +, 567, 752; cds-WP_002918413.1, NZ_KK737548.1, 658283, 658858, +, 576, 66133; cds-WP_002918415.1, NZ_KK737548.1, 658827, 659372, +, 546, 104770; cds-WP _002918417.1, NZ_KK737548.1, 659379, 660104, +, 726, 253516; cds-WP _002918420.1, NZ_KK737548.1, 660152, 661585, +, 1434, 121513; cds-WP _002918423.1, NZ_KK737548.1, 661608, 661895, +, 288, 43629; cds-WP _004144926.1, NZ_KK737548.1, 661966, 662454, +, 489, 34804; cds-WP _002918428.1, NZ_KK737548.1, 662500, 663354, +, 855, 91008; cds-WP _002918431.1, NZ_KK737548.1, 663351, 663623, +, 273, 10476; cds-WP_032430141.1, NZ_KK737548.1, 663687, 664412, -, 726, 925; cds-WP_004889584.1, NZ_KK737548.1, 664409, 665062, -, 654, 255; cds-WP_002918444.1, NZ_KK737548.1, 665296, 667635, -, 2340, 65653; cds-WP _021440775.1, NZ_KK737548.1, 667782, 668846, -, 1065, 2259; cds-WP _032428420.1, NZ_KK737548.1, 669143, 670159, +, 1017, 0; cds-AF52_RS07625, NZ_KK737548.1, 670245, 670964, +, 720, 0; cds-WP _032430142.1, NZ_KK737548.1, 670997, 671650, +, 654, 3379; cds-WP_032175380.1, NZ_KK737548.1, 671925, 672273, +, 349, 1176; cds-AF52_RS07610, NZ_KK737548.1, 672318, 672869, -, 552, 26; cds-WP_004144865.1, NZ_KK737548.1, 672974, 673870, +, 897, 757; cds-WP_002917923.1, NZ_KK737548.1, 673912, 674280, -, 369, 8; cds-WP_002917922.1, NZ_KK737548.1, 674407, 675393, -, 987,118; cds-WP _002917920.1, NZ_KK737548.1, 675474, 675866, -, 393, 0; cds-WP_004181402.1, NZ_KK737548.1, 676212, 676508, -, 297, 0; cds-WP_002917917.1, NZ_KK737548.1, 676505, 676903, -, 399, 252; cds-WP _002917916.1, NZ_KK737548.1, 676906, 677211, -, 306, 179; cds-WP_002917914.1, NZ_KK737548.1, 677257, 677625, -, 369, 19049; cds-WP_004218213.1, NZ_KK737548.1, 677775, 678152, -, 378, 5184; cds-WP_002917909.1, NZ_KK737548.1, 678161, 678823, -, 663, 83; cds-WP _002917907.1, NZ_KK737548.1, 679164, 679940, -, 777, 229; cds-WP_002917905.1, NZ_KK737548.1, 680068, 681369, -, 1302, 150; cds-WP_050484286.1, NZ_KK737548.1, 681850, 683202, +, 1353, 10345; cds-WP_002917898.1, NZ_KK737548.1, 683281, 684768, +, 1488, 43; cds-WP _002917897.1, NZ_KK737548.1, 684898, 685452, +, 555, 9787; cds-WP_002917895.1, NZ_KK737548.1, 685468, 686715, -, 1248, 11465; cds-WP _004185787.1, NZ_KK737548.1, 686982, 687953, -, 972, 49; cds-WP_004181399.1, NZ_KK737548.1, 688225, 689214, -, 990, 23963; cds-WP_004144843.1, NZ_KK737548.1, 689288, 689788, -, 501, 7; cds-WP _021440752.1, NZ_KK737548.1, 689808, 690410, -, 603, 75; cds-WP_002917889.1, NZ_KK737548.1, 690454, 691161, +, 708, 5; cds-WP _021440751.1, NZ_KK737548.1, 691247, 692377, +, 1131, 984; cds-WP_004900808.1, NZ_KK737548.1, 692486, 694507, -, 2022, 8924; cds-WP _019704938.1, NZ_KK737548.1, 694692, 696290, -, 1599, 2; cds-WP_004144837.1, NZ_KK737548.1, 696328, 697299, -, 972, 8; cds-WP_022615670.1, NZ_KK737548.1, 697518, 699005, +, 1488, 0; cds-WP_004174225.1, NZ_KK737548.1, 699002, 700036, +, 1035, 22887; cds-WP_032430144.1, NZ_KK737548.1, 700037, 701035, +, 999, 70; cds-WP _032430145.1, NZ_KK737548.1, 701037, 702038, +, 1002, 83; cds-WP_002917724.1, NZ_KK737548.1, 702050, 702937, +, 888, 100; cds-WP _002917723.1, NZ_KK737548.1, 702934, 703230, +, 297, 0; cds-WP_023316052.1, NZ_KK737548.1, 703277, 704656, -, 1380, 1; cds-WP_002917718.1, NZ_KK737548.1, 704914, 705468, -, 555, 0; cds-WP _021440748.1, NZ_KK737548.1, 705706, 706482, +, 777, 27733; cds-WP _004174228.1, NZ_KK737548.1, 706638, 708287, -, 1650, 23; cds-WP_004174229.1, NZ_KK737548.1, 708363, 709784, -, 1422, 4130; cds-WP _004181378.1, NZ_KK737548.1, 709795, 710427, -, 633, 12988; cds-WP_002917682.1, NZ_KK737548.1, 710438, 711508, -, 1071, 8330; cds-WP_020324489.1, NZ_KK737548.1, 711664, 711849, -, 186, 0; cds-WP _002917681.1, NZ_KK737548.1, 712119, 713216, +, 1098, 571; cds-WP _002917680.1, NZ_KK737548.1, 713318, 715243, +, 1926, 136; cds-WP_002917679.1, NZ_KK737548.1, 715221, 715751, -, 531, 0; cds-WP_021440747.1, NZ_KK737548.1, 715752, 716105, -, 354, 0; cds-WP _032430191.1, NZ_KK737548.1, 716126, 717277, -, 1152, 0; cds-WP_013815099.1-55, NZ_KK737548.1, 717293, 718261, +, 969, 3565; cds-WP _015875176.1, NZ_KK737548.1, 718377, 718805, -, 429, 0; cds-WP_002917676.1, NZ_KK737548.1, 719219, 720886, +, 1668, 9; cds-WP_021440746.1, NZ_KK737548.1, 720899, 721483, +, 585, 0; cds-WP _002917670.1, NZ_KK737548.1, 721486, 721911, +, 426, 0; cds-AF52_RS0124235, NZ_KK737548.1, 721924, 723747, +, 1824, 41; cds-WP _015959037.1, NZ_KK737548.1, 723801, 724604, +, 804, 0; cds-WP _002917655.1, NZ_KK737548.1, 724656, 724961, -, 306, 0; cds-WP _002917651.1, NZ_KK737548.1, 724988, 725563, -, 576, 0; cds-WP _004144810.1, NZ_KK737548.1, 725804, 726466, +, 663, 0; cds-AF52_RS07345, NZ_KK737548.1, 726557, 728848, -, 2292, 0; cds-WP_013815099.1-56, NZ_KK737548.1, 728883, 729851, +, 969, 4849; cds-AF52_RS07335, NZ_KK737548.1, 729909, 730865, -, 957, 43; cds-WP_004174237.1, NZ_KK737548.1, 730870, 731484, -, 615, 0; cds-WP_021440741.1, NZ_KK737548.1, 731785, 732150, +, 366, 0; cds-WP _023313669.1, NZ_KK737548.1, 732219, 732491, -, 273, 0; cds-AF52_RS27330, NZ_KK737548.1, 732623, 732801, +, 179, 256; cds-AF52_RS27335, NZ_KK737548.1, 735673, 735777, -, 105, 0; cds-WP _004152286.1, NZ_KK737548.1, 736364, 737446, +, 1083, 2270; cds-WP _032430148.1, NZ_KK737548.1, 737568, 740642, +, 3075, 259161; cds-WP_023158026.1, NZ_KK737548.1, 740694, 741947, +, 1254, 40958; cds-AF52_RS25890, NZ_KK737548.1, 742013, 742153, +, 141, 0; cds-AF52_RS25895, NZ_KK737548.1, 742168, 742863, -, 696, 3; cds-AF52_RS25905, NZ_KK737548.1, 742956, 743322, +, 367, 1; cds-AF52_RS07290, NZ_KK737548.1, 743401, 743690, +, 290, 3; cds-WP _032430151.1, NZ_KK737548.1, 746080, 747084, +, 1005, 341; cds-AF52_RS07280, NZ_KK737548.1, 747266, 747803, -, 538, 10870; cds-AF52_RS25915, NZ_KK737548.1, 748423, 748868, -, 446, 106; cds-WP_004118832.1, NZ_KK737548.1, 749044, 750777, -, 1734, 37470; cds-WP_004118225.1, NZ_KK737548.1, 750785, 751732, -, 948, 42484; cds-WP_004152278.1, NZ_KK737548.1, 751777, 753381, -, 1605, 667; cds-WP_004118227.1, NZ_KK737548.1, 753394, 754314, -, 921, 0; cds-WP_004118228.1, NZ_KK737548.1, 754314, 755162, -, 849, 343; cds-WP_004118229.1, NZ_KK737548.1, 755159, 755752, -, 594, 2; cds-WP_004118840.1, NZ_KK737548.1, 755749, 756876, -, 1128, 266; cds-WP_004118231.1, NZ_KK737548.1, 757161, 757328, -, 168, 0; cds-WP_162551856.1, NZ_KK737548.1, 757257, 757469, +, 213, 0; cds-AF52_RS07235, NZ_KK737548.1, 757470, 757955, +, 486, 0; cds-WP_004197062.1, NZ_KK737548.1, 760634, 761155, +, 522, 1080; cds-WP_004197067.1, NZ_KK737548.1, 761152, 762105, +, 954, 8; cds-WP_032430155.1, NZ_KK737548.1, 762198, 764522, +, 2325, 1058535; cds-WP_023157913.1, NZ_KK737548.1, 764567, 765469, +, 903, 150849; cds-WP _032430156.1, NZ_KK737548.1, 765466, 766464, +, 999, 42033; cds-WP_004118246.1, NZ_KK737548.1, 766461, 767417, +, 957, 11037; cds-WP_004152282.1, NZ_KK737548.1, 767418, 768185, +, 768, 10521; cds-WP_004118251.1, NZ_KK737548.1, 768284, 768577, +, 294, 44; cds-AF52_RS25920, NZ_KK737548.1, 768652, 768879, +, 228, 3; cds-WP _020806188.1-5, NZ_KK737548.1, 769118, 770098, -, 981, 6587; cds-WP _032430158.1, NZ_KK737548.1, 770398, 771066, +, 669, 0; cds-WP_002885079.1, NZ_KK737548.1, 771455, 772804, +, 1350, 51; cds-WP_002885082.1, NZ_KK737548.1, 772952, 774598, +, 1647, 5836; cds-WP_002885085.1, NZ_KK737548.1, 774761, 775642, -, 882, 2; cds-WP_002885086.1, NZ_KK737548.1, 775748, 776155, +, 408, 0; cds-WP _002885088.1, NZ_KK737548.1, 776152, 776841, +, 690, 0; cds-AF52_RS0124245, NZ_KK737548.1, 777352, 777909, +, 558, 3; cds-WP_004210006.1, NZ_KK737548.1, 777970, 778659, +, 690, 9; cds-WP _032428472.1, NZ_KK737548.1, 778613, 781192, +, 2580, 5; cds-WP _004151734.1, NZ_KK737548.1, 781228, 781776, +, 549, 9; cds-WP _002885139.1, NZ_KK737548.1, 781796, 782893, +, 1098, 645; cds-WP_024623073.1, NZ_KK737548.1, 782941, 784593, -, 1653, 11697; cds-WP_004177812.1, NZ_KK737548.1, 784593, 784904, -, 312, 2016; cds-WP_002885145.1, NZ_KK737548.1, 785088, 787046, -, 1959, 366880; cds-WP_004146658.1, NZ_KK737548.1, 787147, 787347, +, 201, 0; cds-WP_004146659.1, NZ_KK737548.1, 787459, 788772, +, 1314, 1769; cds-WP_004151733.1, NZ_KK737548.1, 788809, 789495, -, 687, 21; cds-WP _012737173.1, NZ_KK737548.1, 789725, 791872, -, 2148, 2; cds-WP _023159071.1, NZ_KK737548.1, 792145, 792873, +, 729, 7539; cds-WP _004177809.1, NZ_KK737548.1, 792959, 793717, +, 759, 22; cds-WP _002885155.1, NZ_KK737548.1, 793723, 794640, +, 918, 1124; cds-AF52_RS0124265, NZ_KK737548.1, 794765, 795772, -, 1008, 71; cds-WP _002885156.1, NZ_KK737548.1, 795847, 796788, -, 942, 6; cds-WP_002885172.1, NZ_KK737548.1, 796819, 797817, -, 999, 0; cds-WP_002885173.1, NZ_KK737548.1, 797814, 799334, -, 1521, 0; cds-WP_021441042.1, NZ_KK737548.1, 799514, 801799, +, 2286, 4; cds-WP_032430167.1, NZ_KK737548.1, 801870, 802223, +, 354, 0; cds-WP _004210023.1, NZ_KK737548.1, 802245, 803003, -, 759, 2; cds-WP _004151727.1, NZ_KK737548.1, 803019, 803582, -, 564, 0; cds-WP _009484481.1, NZ_KK737548.1, 803582, 804718, -, 1137, 180; cds-WP_002885196.1, NZ_KK737548.1, 804715, 805395, -, 681, 0; cds-WP _004177787.1, NZ_KK737548.1, 805503, 806261, -, 759, 0; cds-WP_002885202.1, NZ_KK737548.1, 806258, 807106, -, 849, 0; cds-WP_021441044.1, NZ_KK737548.1, 807099, 808163, -, 1065, 0; cds-WP_002885207.1, NZ_KK737548.1, 808164, 808748, -, 585, 0; cds-WP _020802927.1, NZ_KK737548.1, 808745, 809197, -, 453, 0; cds-WP _032428478.1, NZ_KK737548.1, 809198, 809923, -, 726, 0; cds-WP_004177784.1, NZ_KK737548.1, 810079, 810513, -, 435, 93; cds-WP _004177783.1, NZ_KK737548.1, 810742, 811077, -, 336, 1037; cds-WP_002885227.1, NZ_KK737548.1, 811622, 813124, +, 1503, 129074; cds-WP_004177781.1, NZ_KK737548.1, 813174, 814112, -, 939, 2877; cds-WP _032430168.1, NZ_KK737548.1, 814352, 815704, +, 1353, 6; cds-WP _019724884.1, NZ_KK737548.1, 815798, 817213, +, 1416, 159; cds-WP _004146677.1, NZ_KK737548.1, 817219, 817341, +, 123, 2650; cds-WP_004177778.1, NZ_KK737548.1, 817489, 818445, +, 957, 44512; cds-WP _004177777.1, NZ_KK737548.1, 818455, 818826, +, 372, 1; cds-WP _002885324.1, NZ_KK737548.1, 819518, 819823, -, 306, 0; cds-WP _021441050.1, NZ_KK737548.1, 819823, 820740, -, 918, 38; cds-WP_020316611.1, NZ_KK737548.1, 820816, 820929, +, 114, 0; cds-WP _004221988.1, NZ_KK737548.1, 820886, 821569, -, 684, 0; cds-WP_002885391.1, NZ_KK737548.1, 821795, 822580, +, 786, 29; cds-WP _004177775.1, NZ_KK737548.1, 822626, 823075, -, 450, 1; cds-WP _002885393.1, NZ_KK737548.1, 823075, 824403, -, 1329, 1; cds-WP_032430193.1, NZ_KK737548.1, 824396, 826411, -, 2016, 8; cds-WP_072271759.1, NZ_KK737548.1, 826420, 828867, -, 2448, 0; cds-AF52_RS06930, NZ_KK737548.1, 829500, 829859, +, 360, 0; cds-WP _032430171.1, NZ_KK737548.1, 829892, 830562, +, 671, 9245; cds-AF52_RS27350, NZ_KK737548.1, 830663, 830977, +, 315, 6603; cds-AF52_RS0124285, NZ_KK737548.1, 831034, 831249, +, 216, 15; cds-WP_004146686.1, NZ_KK737548.1, 831244, 831366, -, 123, 0; cds-WP_004153693.1, NZ_KK737548.1, 831561, 831851, +, 291, 2; cds-WP _021441054.1, NZ_KK737548.1, 832920, 835007, -, 2088, 216; cds-WP_002885410.1, NZ_KK737548.1, 835075, 835665, -, 591, 36140; cds-WP _032430173.1, NZ_KK737548.1, 835686, 837482, -, 1797, 46; cds-WP_002885422.1, NZ_KK737548.1, 837458, 837781, -, 324, 58; cds-WP _002885423.1, NZ_KK737548.1, 837906, 839207, -, 1302, 4117; cds-WP_002885424.1, NZ_KK737548.1,
839323, 840759, -, 1437, 373380; cds-WP _002885425.1, NZ_KK737548.1, 841097, 841573, +, 477, 17; cds-WP _009485424.1, NZ_KK737548.1, 841625, 842875, -, 1251, 1; cds-WP_004152420.1, NZ_KK737548.1, 843108, 843401, +, 294, 74304; cds-WP _002885441.1, NZ_KK737548.1, 843439, 845085, +, 1647, 1027168; cds-AF52_RS27360, NZ_KK737548.1, 846097, 846318, -, 222, 0; cds-WP_013815099.1-57, NZ_KK737548.1, 846353, 847321, +, 969, 2482; cds-AF52_RS25950, NZ_KK737548.1, 847378, 848127, -, 750, 0; cds-WP_004186605.1, NZ_KK737548.1, 848177, 848857, -, 681, 0; cds-WP _004186607.1, NZ_KK737548.1, 848908, 849450, -, 543, 0; cds-WP _004222578.1, NZ_KK737549.1, 190, 258, +, 69, 1; cds-WP _002887848.1, NZ_KK737549.1, 340, 2802, +, 2463, 27500; cds-WP_002887853.1, NZ_KK737549.1, 2804, 3733, +, 930, 27; cds-WP _002887863.1, NZ_KK737549.1, 3737, 5017, +, 1281, 10344; cds-WP _004222583.1, NZ_KK737549.1, 5332, 5709, +, 378, 3; cds-WP _004178561.1, NZ_KK737549.1, 5778, 6551, -, 774, 11494; cds-WP_002887871.1, NZ_KK737549.1, 6629, 8059, -, 1431, 9105; cds-WP_002887897.1, NZ_KK737549.1, 8262, 9215, +, 954, 221510; cds-WP _002887898.1, NZ_KK737549.1, 9315, 9902, +, 588, 404; cds-WP_021441173.1, NZ_KK737549.1, 10022, 11326, +, 1305, 2411; cds-WP_004177461.1, NZ_KK737549.1, 11389, 11955, -, 567, 4285; cds-WP _021441175.1, NZ_KK737549.1, 12044, 12793, -, 750, 0; cds-WP_002887909.1, NZ_KK737549.1, 12821, 13225, -, 405, 0; cds-WP_004146997.1, NZ_KK737549.1, 13611, 15527, +, 1917, 133683; cds-WP_002887955.1, NZ_KK737549.1, 15615, 16748, +, 1134, 7041; cds-WP _004192071.1, NZ_KK737549.1, 16926, 18101, +, 1176, 44; cds-WP_002887961.1, NZ_KK737549.1, 18156, 19052, +, 897, 249; cds-WP_002887965.1, NZ_KK737549.1, 19172, 19435, -, 264, 30932; cds-WP _002887969.1, NZ_KK737549.1, 19765, 20703, +, 939, 183083; cds-WP_032430302.1, NZ_KK737549.1, 20747, 23563, +, 2817, 229571; cds-WP _002887973.1, NZ_KK737549.1, 23563, 24063, +, 501, 23780; cds-WP_002887976.1, NZ_KK737549.1, 24179, 24628, +, 450, 50470; cds-WP_002887979.1, NZ_KK737549.1, 24630, 25580, +, 951, 37163; cds-WP_032430200.1, NZ_KK737549.1, 25647, 26561, +, 915, 3437; cds-WP_032428524.1, NZ_KK737549.1, 26592, 26957, +, 366, 53; cds-WP _021441178.1, NZ_KK737549.1, 26905, 27834, -, 930, 54; cds-WP_004192057.1, NZ_KK737549.1, 27927, 28556, +, 630, 4116; cds-WP _004222618.1, NZ_KK737549.1, 28562, 29266, -, 705, 30265; cds-WP _004151379.1, NZ_KK737549.1, 29259, 30887, -, 1629, 2075; cds-WP_032430201.1, NZ_KK737549.1, 31059, 32360, -, 1302, 0; cds-WP _021441180.1, NZ_KK737549.1, 32376, 34154, -, 1779, 0; cds-WP _012540458.1, NZ_KK737549.1, 34170, 34421, -, 252, 0; cds-WP_004151375.1, NZ_KK737549.1, 34577, 35917, -, 1341, 2; cds-WP _004222626.1, NZ_KK737549.1, 36174, 37202, +, 1029, 0; cds-WP _004151373.1, NZ_KK737549.1, 37233, 37526, +, 294, 0; cds-WP _004151372.1, NZ_KK737549.1, 37523, 38392, +, 870, 0; cds-WP_032428528.1, NZ_KK737549.1, 38403, 39929, +, 1527, 236; cds-WP_004222628.1, NZ_KK737549.1, 39948, 40856, +, 909, 0; cds-WP _021441183.1, NZ_KK737549.1, 41085, 41906, +, 822, 12843; cds-WP_004212571.1, NZ_KK737549.1, 42364, 43512, +, 1149, 163662; cds-WP _002888056.1, NZ_KK737549.1, 43530, 46754, +, 3225, 888223; cds-WP _002888059.1, NZ_KK737549.1, 46924, 47157, +, 234, 0; cds-WP_002888068.1, NZ_KK737549.1, 47289, 47822, +, 534, 53984; cds-WP_004145992.1, NZ_KK737549.1, 47815, 49680, +, 1866, 5627; cds-WP_002888320.1, NZ_KK737549.1, 49854, 50333, +, 480, 570; cds-WP_002888321.1, NZ_KK737549.1, 50586, 51434, -, 849, 519; cds-WP _002888323.1, NZ_KK737549.1, 51439, 51816, -, 378, 37828; cds-WP_002888324.1, NZ_KK737549.1, 51819, 52640, -, 822, 100528; cds-WP_032430205.1, NZ_KK737549.1, 52637, 53626, -, 990, 31067; cds-WP_002888329.1, NZ_KK737549.1, 53632, 54918, -, 1287, 80546; cds-WP_032103829.1, NZ_KK737549.1, 54973, 57321, -, 2349, 32646; cds-WP_002888332.1, NZ_KK737549.1, 57565, 58392, +, 828, 1392; cds-WP_002888337.1, NZ_KK737549.1, 58425, 59123, -, 699, 1201; cds-WP_004192048.1, NZ_KK737549.1, 59113, 60738, -, 1626, 53355; cds-WP _002888345.1, NZ_KK737549.1, 61063, 62427, +, 1365, 114; cds-WP_021441189.1, NZ_KK737549.1, 62607, 63146, +, 540, 39; cds-WP_002888348.1, NZ_KK737549.1, 63209, 63868, -, 660, 0; cds-WP_032428530.1, NZ_KK737549.1, 63881, 66787, -, 2907, 61095; cds-WP _021441191.1, NZ_KK737549.1, 66975, 69332, -, 2358, 401; cds-WP _002888353.1, NZ_KK737549.1, 69499, 70194, -, 696, 0; cds-WP_021441192.1, NZ_KK737549.1, 70332, 71834, -, 1503, 81; cds-WP_023279864.1, NZ_KK737549.1, 71845, 73554, -, 1710, 0; cds-WP _004145972.1, NZ_KK737549.1, 73892, 74737, +, 846, 14290; cds-WP_002888373.1, NZ_KK737549.1, 74866, 75633, +, 768, 7957; cds-WP_032430207.1, NZ_KK737549.1, 75620, 76321, -, 702, 2; cds-WP_002888430.1, NZ_KK737549.1, 76305, 77915, -, 1611, 10814; cds-WP_032428532.1, NZ_KK737549.1, 77891, 78874, -, 984, 39879; cds-AF52_RS0124360, NZ_KK737549.1, 79164, 80819, -, 1656, 781; cds-WP_002888448.1, NZ_KK737549.1, 80907, 81059, +, 153, 1298; cds-WP_004147046.1, NZ_KK737549.1, 81327, 82511, +, 1185, 8898; cds-WP_004147047.1, NZ_KK737549.1, 82545, 83855, -, 1311, 91; cds-WP _013815099.1-58, NZ_KK737549.1, 84110, 85078, +, 969, 4306; cds-WP _002888455.1, NZ_KK737549.1, 85153, 86049, +, 897, 0; cds-WP_004177420.1, NZ_KK737549.1, 86087, 86842, -, 756, 67; cds-WP_032428534.1, NZ_KK737549.1, 86873, 88087, -, 1215, 11; cds-WP_002888460.1, NZ_KK737549.1, 88098, 88961, -, 864, 0; cds-WP_004147052.1, NZ_KK737549.1, 88973, 91006, -, 2034, 12; cds-WP _002888481.1, NZ_KK737549.1, 91402, 92007, -, 606, 8; cds-WP_021441196.1, NZ_KK737549.1, 92018, 93418, -, 1401, 0; cds-WP_002888487.1, NZ_KK737549.1, 93421, 94512, -, 1092, 263; cds-WP _023287248.1, NZ_KK737549.1, 94512, 96083, -, 1572, 4; cds-WP _004222662.1, NZ_KK737549.1, 96179, 96265, -, 87, 0; cds-WP_148673689.1, NZ_KK737549.1, 96879, 97847, +, 969, 0; cds-WP_002888534.1, NZ_KK737549.1, 98242, 99966, +, 1725, 4109; cds-WP _002888536.1, NZ_KK737549.1, 99969, 100460, +, 492, 117; cds-WP_004145952.1, NZ_KK737549.1, 100641, 101645, +, 1005, 76000; cds-WP _002888541.1, NZ_KK737549.1, 102286, 102744, +, 459, 675; cds-WP_002888547.1, NZ_KK737549.1, 102747, 103688, +, 942, 15637; cds-WP_002888550.1, NZ_KK737549.1, 103685, 104050, +, 366, 12599; cds-WP_032430210.1, NZ_KK737549.1, 104066, 105832, +, 1767, 25195; cds-WP_004182923.1, NZ_KK737549.1, 105819, 107306, +, 1488, 23222; cds-WP_004177411.1, NZ_KK737549.1, 107303, 108661, +, 1359, 14166; cds-WP_002888562.1, NZ_KK737549.1, 108655, 109737, +, 1083, 25753; cds-WP _004151364.1, NZ_KK737549.1, 109740, 111056, +, 1317, 547; cds-WP _002888565.1, NZ_KK737549.1, 111056, 112330, +, 1275, 1784; cds-WP_002888566.1, NZ_KK737549.1, 112327, 113397, +, 1071, 16058; cds-WP _004145947.1, NZ_KK737549.1, 113445, 114920, +, 1476, 50440; cds-WP _002888623.1, NZ _KK737549.1, 114913, 115833, +, 921, 10736; cds-WP _002888624.1, NZ_KK737549.1, 115835, 116665, +, 831, 20887; cds-WP_002888625.1, NZ _KK737549.1, 116662, 117924, +, 1263, 753; cds-WP_002888628.1, NZ_KK737549.1, 117987, 119138, +, 1152, 53387; cds-WP_032430211.1, NZ_KK737549.1, 119239, 120156, +, 918, 253697; cds-WP_004152922.1, NZ_KK737549.1, 120473, 120976, +, 504, 19241; cds-WP_002888638.1, NZ_KK737549.1, 121039, 123744, +, 2706, 370239; cds-WP _004145942.1, NZ_KK737549.1, 123803, 124195, +, 393, 3515; cds-WP_002888644.1, NZ_KK737549.1, 124248, 124442, -, 195, 2111; cds-WP _004178596.1, NZ_KK737549.1, 124452, 125195, -, 744, 19502; cds-WP_002888649.1, NZ_KK737549.1, 125195, 125815, -, 621, 39339; cds-WP _004145938.1, NZ_KK737549.1, 126159, 127202, +, 1044, 10966; cds-WP_021441203.1, NZ_KK737549.1, 127239, 128444, -, 1206, 0; cds-WP _004145935.1, NZ_KK737549.1, 128434, 129819, -, 1386, 0; cds-WP _002888683.1, NZ_KK737549.1, 129830, 130261, -, 432, 0; cds-WP _002888684.1, NZ_KK737549.1, 130443, 131336, -, 894, 30; cds-WP_002888692.1, NZ_KK737549.1, 131470, 132033, +, 564, 106; cds-WP_002888694.1, NZ_KK737549.1, 132030, 132884, +, 855, 1498; cds-WP_021441204.1, NZ_KK737549.1, 133120, 134070, -, 951, 51; cds-WP _032428537.1, NZ_KK737549.1, 134070, 135476, -, 1407, 115; cds-WP_004147090.1, NZ_KK737549.1, 135661, 137031, -, 1371, 26; cds-WP _002888699.1, NZ_KK737549.1, 137675, 138454, +, 780, 336; cds-WP_032430213.1, NZ_KK737549.1, 138591, 141254, +, 2664, 828611; cds-WP _021441205.1, NZ_KK737549.1, 141269, 143167, +, 1899, 122054; cds-WP _002888731.1, NZ_KK737549.1, 143374, 144798, +, 1425, 765442; cds-WP_004177403.1, NZ_KK737549.1, 144963, 145247, +, 285, 19; cds-WP _002888733.1, NZ_KK737549.1, 145293, 146099, -, 807, 0; cds-WP _032430214.1, NZ_KK737549.1, 146111, 147721, -, 1611, 18; cds-WP _002888735.1, NZ_KK737549.1, 148153, 150750, +, 2598, 2880974; cds-WP _002888737.1, NZ_KK737549.1, 150937, 151299, +, 363, 1662; cds-WP_002888739.1, NZ_KK737549.1, 151372, 152166, -, 795, 491; cds-WP _002888741.1, NZ_KK737549.1, 152191, 153051, -, 861, 24090; cds-WP_072072754.1, NZ_KK737549.1, 153201, 154394, -, 1194, 6; cds-AF52_RS28060, NZ_KK737549.1, 154404, 154643, -, 240, 0; cds-WP _002888804.1, NZ_KK737549.1, 155225, 155635, -, 411, 0; cds-WP_002888808.1, NZ_KK737549.1, 155882, 156229, -, 348, 31; cds-WP _032430215.1, NZ_KK737549.1, 156375, 157973, +, 1599, 11372; cds-WP _002888816.1, NZ_KK737549.1, 158057, 160447, -, 2391, 27582; cds-WP _012542835.1, NZ_KK737549.1, 160528, 160647, +, 120, 0; cds-WP _002888819.1, NZ _KK737549.1, 160652, 161188, +, 537, 2461; cds-WP_002888821.1, NZ_KK737549.1, 161248, 161910, -, 663, 7361; cds-WP _002888823.1, NZ_KK737549.1, 162095, 163021, +, 927, 475; cds-WP _004204389.1, NZ_KK737549.1, 163018, 163788, +, 771, 353; cds-WP_023282187.1, NZ_KK737549.1, 163911, 164351, +, 441, 12; cds-WP _004145907.1, NZ _KK737549.1, 164410, 165663, +, 1254, 12146; cds-WP_002888834.1, NZ_KK737549.1, 165699, 166079, -, 381, 1723; cds-WP_002888835.1, NZ_KK737549.1, 166172, 167026, -, 855, 10982; cds-WP_002888841.1, NZ_KK737549.1, 167038, 167829, -, 792, 5032; cds-WP_004145905.1, NZ_KK737549.1, 167950, 168429, -, 480, 1550; cds-WP _071526609.1, NZ _KK737549.1, 168426, 169820, -, 1395, 16236; cds-WP _021441212.1, NZ_KK737549.1, 169883, 170764, -, 882, 39309; cds-WP _002888845.1, NZ_KK737549.1, 170824, 171279, -, 456, 64328; cds-WP _032430216.1, NZ_KK737549.1, 171442, 172158, -, 717, 414; cds-WP_004145901.1, NZ_KK737549.1, 172158, 172694, -, 537, 2; cds-WP _023282188.1, NZ_KK737549.1, 172767, 175196, +, 2430, 93160; cds-WP _021441214.1, NZ_KK737549.1, 175244, 176044, -, 801, 81; cds-WP _021441215.1, NZ_KK737549.1, 176041, 176787, -, 747, 0; cds-WP_004151943.1, NZ_KK737549.1, 176777, 177262, -, 486, 0; cds-WP _021441216.1, NZ_KK737549.1, 177255, 178448, -, 1194, 0; cds-WP _004183041.1, NZ _KK737549.1, 178435, 179421, -, 987, 37; cds-WP_032428541.1, NZ_KK737549.1, 179418, 180014, -, 597, 0; cds-WP _002889025.1, NZ_KK737549.1, 180011, 180376, -, 366, 0; cds-WP_021441218.1, NZ_KK737549.1, 180379, 180888, -, 510, 0; cds-WP _002889054.1, NZ_KK737549.1, 180888, 181310, -, 423, 131; cds-WP _004177382.1, NZ_KK737549.1, 181331, 182545, -, 1215, 6; cds-WP_004145888.1, NZ_KK737549.1, 182547, 184040, -, 1494, 4493; cds-WP _032430217.1, NZ_KK737549.1, 184037, 186010, -, 1974, 72; cds-WP_004177377.1, NZ_KK737549.1, 186020, 186862, -, 843, 3250; cds-WP _004219434.1, NZ_KK737549.1, 186847, 186984, -, 138, 2; cds-WP _023286046.1, NZ_KK737549.1, 187169, 190477, +, 3309, 1465; cds-WP_021441221.1, NZ_KK737549.1, 190734, 191291, +, 558, 0; cds-WP _004194926.1, NZ_KK737549.1, 191330, 191704, -, 375, 107; cds-WP_004147125.1, NZ_KK737549.1, 191767, 191901, -, 135, 0; cds-WP _002889208.1, NZ_KK737549.1, 192292, 194844, +, 2553, 1083; cds-WP _004178624.1, NZ_KK737549.1, 195079, 197286, +, 2208, 9555; cds-WP _002889212.1, NZ_KK737549.1, 197332, 198129, +, 798, 14123; cds-WP _004178627.1, NZ_KK737549.1, 198129, 199019, +, 891, 28095; cds-WP_032430218.1, NZ_KK737549.1, 199016, 200998, +, 1983, 328; cds-WP_002889272.1, NZ_KK737549.1, 201135, 202415, -, 1281, 42656; cds-WP _004151932.1, NZ_KK737549.1, 202596, 204014, +, 1419, 7; cds-WP_002889275.1, NZ_KK737549.1, 204096, 204440, +, 345, 39; cds-WP _002889277.1, NZ_KK737549.1, 204512, 205135, -, 624, 1529; cds-WP _002889279.1, NZ_KK737549.1, 205172, 205972, -, 801, 42804; cds-WP_002889282.1, NZ_KK737549.1, 205965, 206663, -, 699, 77574; cds-WP _004145871.1, NZ_KK737549.1, 206747, 208261, +, 1515, 18; cds-WP_004177363.1, NZ_KK737549.1, 208396, 209829, +, 1434, 34282; cds-WP _002889289.1, NZ_KK737549.1, 209996, 211153, +, 1158, 949; cds-WP _002889292.1, NZ_KK737549.1, 211272, 211658, -, 387, 21241; cds-WP _002889295.1, NZ_KK737549.1, 211766, 212590, -, 825, 118747; cds-
WP_004151931.1, NZ_KK737549.1, 212621, 215284, -, 2664, 40940; cds-WP_004177362.1, NZ_KK737549.1, 215342, 216136, -, 795, 7911; cds-WP_002889299.1, NZ_KK737549.1, 216459, 217184, +, 726, 1233533; cds-WP _004151930.1, NZ_KK737549.1, 217304, 218155, +, 852, 226348; cds-WP_002889306.1, NZ_KK737549.1, 218305, 219030, +, 726, 164210; cds-WP _002889308.1, NZ_KK737549.1, 219181, 219738, +, 558, 126140; cds-WP_004178642.1, NZ_KK737549.1, 219967, 221169, +, 1203, 1492; cds-WP _002889316.1, NZ_KK737549.1, 221407, 222165, +, 759, 36898; cds-WP _032430220.1, NZ_KK737549.1, 222178, 223035, +, 858, 4342; cds-WP _004177358.1, NZ_KK737549.1, 223047, 224399, +, 1353, 42857; cds-WP _004145860.1, NZ_KK737549.1, 224431, 226860, +, 2430, 441178; cds-WP_004178645.1, NZ_KK737549.1, 226982, 227467, +, 486, 339712; cds-WP_002889327.1, NZ_KK737549.1, 227471, 228496, +, 1026, 43278; cds-WP _004145858.1, NZ_KK737549.1, 228602, 229057, +, 456, 54552; cds-WP _004178647.1, NZ_KK737549.1, 229061, 229849, +, 789, 23440; cds-WP _002889374.1, NZ_KK737549.1, 229849, 231000, +, 1152, 2174; cds-WP_021441229.1, NZ_KK737549.1, 230997, 231596, +, 600, 18490; cds-WP_004177353.1, NZ_KK737549.1, 231614, 235096, +, 3483, 17802; cds-WP _004145855.1, NZ_KK737549.1, 235109, 236068, +, 960, 46011; cds-WP_021441230.1, NZ_KK737549.1, 236182, 238335, +, 2154, 1507; cds-WP_021441231.1, NZ_KK737549.1, 238382, 238771, +, 390, 1715; cds-WP 009309280.1, NZ_KK737549.1, 238825, 240138, +, 1314, 1268; cds-WP _002889424.1, NZ_KK737549.1, 240152, 240412, -, 261, 362; cds-WP _002889429.1, NZ_KK737549.1, 240399, 240599, -, 201, 115; cds-WP_009309281.1, NZ_KK737549.1, 240796, 241341, +, 546, 724; cds-WP _002889433.1, NZ_KK737549.1, 241338, 241751, +, 414, 1093; cds-WP_002889435.1, NZ_KK737549.1, 241794, 242492, +, 699, 937; cds-WP _004212714.1, NZ_KK737549.1, 242624, 243466, -, 843, 142; cds-WP _004151928.1, NZ_KK737549.1, 243525, 245243, -, 1719, 98057; cds-WP_032430223.1, NZ_KK737549.1, 245356, 246063, -, 708, 5762; cds-WP_002889443.1, NZ_KK737549.1, 246060, 246467, -, 408, 21380; cds-WP_002889445.1, NZ_KK737549.1, 246575, 247390, -, 816, 1546; cds-WP_002889448.1, NZ_KK737549.1, 247434, 248087, -, 654, 44; cds-WP _002889450.1, NZ_KK737549.1, 248080, 249111, -, 1032, 19; cds-WP_002889452.1, NZ_KK737549.1, 249300, 249866, +, 567, 43; cds-WP _012737430.1, NZ_KK737549.1, 256451, 257254, +, 804, 8; cds-WP_002889616.1, NZ_KK737549.1, 257300, 258205, -, 906, 2; cds-WP_021441232.1, NZ_KK737549.1, 258310, 259494, +, 1185, 6754; cds-WP_002889629.1, NZ_KK737549.1, 259597, 260394, +, 798, 338; cds-WP_021441233.1, NZ_KK737549.1, 260486, 261256, +, 771, 0; cds-WP_002889632.1, NZ_KK737549.1, 261314, 262681, -, 1368, 2567; cds-WP _032428550.1, NZ_KK737549.1, 262753, 263508, -, 756, 16; cds-WP _002889685.1, NZ_KK737549.1, 263541, 264263, +, 723, 1708; cds-WP_002889686.1, NZ_KK737549.1, 264260, 264727, -, 468, 1009; cds-WP_023286054.1, NZ_KK737549.1, 264783, 265523, +, 741, 54; cds-WP_032430225.1, NZ_KK737549.1, 265793, 266674, -, 882,3; cds-WP_004178672.1, NZ_KK737549.1, 266778, 267368, +, 591, 1; cds-WP_002889689.1, NZ_KK737549.1, 267399, 268169, -, 771, 715; cds-WP_021441237.1, NZ_KK737549.1, 268347, 270791, -, 2445, 195997; cds-WP_004145825.1, NZ_KK737549.1, 271031, 271612, +, 582, 73197; cds-WP_002889696.1, NZ_KK737549.1, 271687, 272454, +, 768, 1436; cds-WP _002889699.1, NZ_KK737549.1, 272425, 273165, -, 741, 93659; cds-WP _002889702.1, NZ_KK737549.1, 273577, 274920, +, 1344, 22540; cds-WP _002889703.1, NZ_KK737549.1, 274924, 276162, +, 1239, 26752; cds-WP_004177336.1, NZ_KK737549.1, 276155, 276949, +, 795, 9565; cds-WP_002889716.1, NZ_KK737549.1, 276942, 277580, +, 639, 28021; cds-WP _004177335.1, NZ_KK737549.1, 277587, 278183, +, 597, 6992; cds-WP _021441239.1, NZ_KK737549.1, 278196, 279419, +, 1224, 29093; cds-WP_004178678.1, NZ_KK737549.1, 279422, 279637, +, 216, 20; cds-WP _021441240.1, NZ_KK737549.1, 280018, 280878, -, 861, 13; cds-WP_002889726.1, NZ_KK737549.1, 281038, 282093, +, 1056, 327; cds-WP _002889727.1, NZ_KK737549.1, 282096, 282548, +, 453, 0; cds-WP_004152033.1, NZ_KK737549.1, 282591, 284048, -, 1458, 31423; cds-WP _002889733.1, NZ_KK737549.1, 284306, 284764, +, 459, 5367; cds-WP _002889742.1, NZ_KK737549.1, 284870, 286114, +, 1245, 1297; cds-WP _004177332.1, NZ_KK737549.1, 286172, 286570, +, 399, 8569; cds-WP _004177331.1, NZ_KK737549.1, 286656, 287348, -, 693, 9; cds-WP _032430227.1, NZ_KK737549.1, 287394, 288794, -, 1401, 1; cds-WP_032430228.1, NZ_KK737549.1, 288944, 290227, -, 1284, 1; cds-WP_032430229.1, NZ_KK737549.1, 290240, 291688, -, 1449, 3; cds-WP_002889846.1, NZ_KK737549.1, 291713, 292981, -, 1269, 30; cds-WP_002889847.1, NZ_KK737549.1, 293009, 293986, -, 978, 27; cds-WP_032430230.1, NZ_KK737549.1, 294448, 294705, +, 258, 29; cds-WP_032430232.1, NZ_KK737549.1, 294763, 295512, +, 750, 143; cds-WP_032421047.1, NZ_KK737549.1, 295784, 296248, +, 465, 1639; cds-WP _009309773.1, NZ_KK737549.1, 296439, 297173, -, 735, 0; cds-WP _021441244.1, NZ_KK737549.1, 297139, 298473, -, 1335, 0; cds-WP _032430234.1, NZ_KK737549.1, 298478, 301033, -, 2556, 16; cds-WP_009309770.1, NZ_KK737549.1, 301017, 301775, -, 759, 0; cds-AF52_RS05525, NZ_KK737549.1, 301835, 302236, -, 402, 0; cds-AF52_RS27385, NZ_KK737549.1, 302271, 302442, +, 172, 324; cds-AF52_RS27390, NZ_KK737549.1, 303421, 303558, +, 138, 1896; cds-WP_050484291.1, NZ_KK737549.1, 303624, 304661, +, 1038, 1658; cds-WP _004222859.1, NZ_KK737549.1, 304706, 305272, -, 567, 416; cds-WP_023279253.1, NZ_KK737549.1, 305685, 306554, +, 870, 0; cds-WP_021441254.1, NZ_KK737549.1, 306650, 307315, +, 666, 0; cds-WP_124053765.1, NZ_KK737549.1, 307782, 307952, -, 171, 0; cds-WP _016531458.1, NZ_KK737549.1, 308463, 309347, -, 885, 0; cds-WP_002890001.1, NZ_KK737549.1, 309344, 310216, -, 873, 0; cds-WP _002890002.1, NZ_KK737549.1, 310283, 311221, -, 939, 0; cds-WP_004177295.1, NZ_KK737549.1, 311244, 312086, -, 843, 1; cds-WP _013815099.1-59, NZ_KK737549.1, 312505, 313473, -, 969, 6075; cds-WP _013815099.1-60, NZ_KK737549.1, 313954, 314922, +, 969, 4799; cds-AF52_RS27405, NZ_KK737549.1, 314979, 315062, -, 84, 17; cds-WP _004144574.1, NZ_KK737549.1, 315352, 316455, +, 1104, 28555; cds-WP _004147253.1, NZ_KK737549.1, 316466, 317719, +, 1254, 18911; cds-AF52_RS27410, NZ_KK737549.1, 318130, 318266, -, 137, 699; cds-AF52_RS27415, NZ_KK737549.1, 319963, 320115, -, 153, 207; cds-AF52_RS05440, NZ_KK737549.1, 320150, 320659, +, 510, 0; cds-WP _032430236.1, NZ_KK737549.1, 320718, 321386, +, 669, 69; cds-WP_032430238.1, NZ_KK737549.1, 321412, 323937, +, 2526, 0; cds-WP_002890060.1, NZ_KK737549.1, 323927, 325570, +, 1644, 0; cds-WP 002890061.1, NZ_KK737549.1, 325539, 326249, +, 711, 0; cds-WP_004178714.1, NZ_KK737549.1, 326253, 326942, -, 690, 0; cds-WP_032430239.1, NZ_KK737549.1, 326932, 327684, -, 753, 0; cds-WP _004151355.1, NZ_KK737549.1, 327681, 328499, -, 819, 0; cds-WP _002890106.1, NZ_KK737549.1, 328501, 329310, -, 810, 0; cds-WP_013815099.1-61, NZ_KK737549.1, 329684, 330652, +, 969, 29151; cds-WP _002890108.1, NZ_KK737549.1, 330723, 330893, +, 171, 37579; cds-WP _002890126.1, NZ_KK737549.1, 331010, 331705, -, 696, 0; cds-WP_004151354.1, NZ_KK737549.1, 331698, 332480, -, 783, 0; cds-WP_004144594.1, NZ_KK737549.1, 332467, 333516, -, 1050, 1186; cds-WP_025861891.1, NZ_KK737549.1, 333516, 334415, -, 900, 0; cds-WP _004147267.1, NZ_KK737549.1, 334435, 335541, -, 1107, 0; cds-WP _009485305.1, NZ_KK737549.1, 335534, 335677, +, 144, 0; cds-WP_004147268.1, NZ_KK737549.1, 336117, 336893, -, 777, 0; cds-WP_032430241.1, NZ_KK737549.1, 336890, 338278, -, 1389, 0; cds-WP_032430243.1, NZ_KK737549.1, 338288, 339667, -, 1380, 0; cds-WP_021441263.1, NZ_KK737549.1, 339730, 339858, +, 129, 0; cds-WP_002890161.1, NZ_KK737549.1, 339941, 341350, +, 1410, 6; cds-WP_032430245.1, NZ_KK737549.1, 341337, 342269, +, 933, 66; cds-WP _032430246.1, NZ_KK737549.1, 342509, 343471, +, 963, 0; cds-WP_004183141.1, NZ_KK737549.1, 343483, 344250, +, 768, 0; cds-WP_004144603.1, NZ_KK737549.1, 344247, 345074, +, 828, 0; cds-WP_004147277.1, NZ_KK737549.1, 345071, 345922, +, 852, 0; cds-WP _032430247.1, NZ_KK737549.1, 346096, 347814, -, 1719, 3; cds-WP _002890201.1, NZ_KK737549.1, 348267, 349241, -, 975, 11124; cds-WP_004151350.1, NZ_KK737549.1, 349348, 350508, -, 1161, 194; cds-WP _004151349.1, NZ_KK737549.1, 350727, 351239, +, 513, 49831; cds-WP _002890204.1, NZ_KK737549.1, 351357, 352577, +, 1221, 473; cds-WP_004183146.1, NZ_KK737549.1, 352596, 353702, +, 1107, 19; cds-WP_002890210.1, NZ_KK737549.1, 353699, 354001, -, 303, 5; cds-WP_002890215.1, NZ_KK737549.1, 354254, 354457, +, 204, 360; cds-WP_004151348.1, NZ_KK737549.1, 354490, 355587, -, 1098, 53634; cds-WP_004212277.1, NZ_KK737549.1, 355687, 356370, +, 684, 0; cds-WP_002890219.1, NZ_KK737549.1, 356540, 357739, +, 1200, 0; cds-WP _002890221.1, NZ_KK737549.1, 358039, 358299, +, 261, 0; cds-WP_004232385.1, NZ_KK737549.1, 358389, 359819, +, 1431, 3256; cds-WP _002890225.1, NZ_KK737549.1, 359911, 360228, +, 318, 1; cds-WP_004177276.1, NZ_KK737549.1, 360296, 361105, -, 810, 4565; cds-WP _002890230.1, NZ_KK737549.1, 361222, 361680, +, 459, 0; cds-WP _032430252.1, NZ_KK737549.1, 361677, 362387, -, 711, 312; cds-WP_002890276.1, NZ_KK737549.1, 362716, 363249, +, 534, 322; cds-WP _002890278.1, NZ_KK737549.1, 363321, 363512, +, 192, 27; cds-WP_004144619.1, NZ_KK737549.1, 363763, 364443, +, 681, 2; cds-WP _002890284.1, NZ_KK737549.1, 364521, 364805, +, 285, 4661; cds-WP_002890285.1, NZ_KK737549.1, 364877, 365788, -, 912, 11923; cds-WP_032430253.1, NZ_KK737549.1, 365880, 366794, +, 915, 16; cds-WP _032430254.1, NZ_KK737549.1, 366868, 370005, -, 3138, 977; cds-WP _032430256.1, NZ_KK737549.1, 370002, 371207, -, 1206, 28; cds-WP_002890343.1, NZ_KK737549.1, 371390, 372079, +, 690, 12; cds-WP_002890344.1, NZ_KK737549.1, 372101, 373396, +, 1296, 5361; cds-WP_032430258.1, NZ_KK737549.1, 373810, 375129, +, 1320, 32171; cds-WP_002890348.1, NZ_KK737549.1, 375216, 376589, +, 1374, 47; cds-WP _032430260.1, NZ_KK737549.1, 376741, 378558, +, 1818, 2667; cds-WP _002890353.1, NZ_KK737549.1, 378555, 379457, -, 903, 5; cds-WP _002890357.1, NZ_KK737549.1, 379724, 380365, +, 642, 0; cds-WP_002890365.1, NZ_KK737549.1, 380552, 381097, +, 546, 100; cds-WP _023286084.1, NZ_KK737549.1, 381122, 382990, +, 1869, 0; cds-WP_004218830.1, NZ_KK737549.1, 383049, 383903, -, 855, 0; cds-WP_032430262.1, NZ_KK737549.1, 383939, 384883, -, 945, 0; cds-WP_013815099.1-62, NZ_KK737549.1, 384903, 385871, +, 969, 5529; cds-AF52_RS05135, NZ_KK737549.1, 386033, 387202, -, 1170, 4; cds-WP _013815099.1-63, NZ_KK737549.1, 387236, 388204, +, 969, 5618; cds-AF52_RS28095, NZ_KK737549.1, 388263, 388406, +, 144, 24; cds-WP _019704181.1, NZ_KK737549.1, 388474, 388707, +, 234, 0; cds-WP _032430264.1, NZ_KK737549.1, 388707, 388934, +, 228, 0; cds-AF52_RS05115, NZ_KK737549.1, 388931, 389551, +, 621, 12; cds-WP _013815099.1-64, NZ_KK737549.1, 389609, 390577, -, 969, 3658; cds-WP_021441029.1, NZ_KK737549.1, 390933, 392513, +, 1581, 0; cds-WP _004151744.1, NZ_KK737549.1, 392638, 393162, -, 525, 13884; cds-WP _004146620.1, NZ_KK737549.1, 393414, 396239, +, 2826, 23793; cds-WP_002884953.1, NZ_KK737549.1, 396240, 396596, -, 357, 3; cds-WP _002884951.1, NZ_KK737549.1, 396600, 397016, -, 417, 43; cds-WP _004151745.1, NZ_KK737549.1, 397147, 397860, -, 714, 115; cds-WP _009485000.1, NZ_KK737549.1, 398051, 399244, -, 1194, 18548; cds-WP_004214117.1, NZ_KK737549.1, 399421, 400500, -, 1080, 680; cds-WP_002884942.1, NZ_KK737549.1, 400532, 401947, -, 1416, 19636; cds-WP_032428470.1, NZ_KK737549.1, 402117, 403100, +, 984, 1; cds-WP _009484997.1, NZ_KK737549.1, 403282, 403524, -, 243, 0; cds-WP_004214118.1, NZ_KK737549.1, 403664, 404662, -, 999, 701; cds-WP _002884935.1, NZ_KK737549.1, 404763, 405251, -, 489, 201; cds-WP_002884931.1, NZ_KK737549.1, 405501, 406016, +, 516, 35255; cds-WP_004151747.1, NZ_KK737549.1, 406112, 406321, -, 210, 3; cds-WP _004219133.1, NZ_KK737549.1, 406387, 406536, +, 150, 1; cds-WP_004151748.1, NZ_KK737549.1, 406556, 407872, -, 1317, 13275; cds-WP_002884822.1, NZ_KK737549.1, 407942, 408550, -, 609, 20014; cds-WP_002884819.1, NZ_KK737549.1, 408673, 409041, -, 369, 4; cds-WP_004178269.1, NZ_KK737549.1, 409170, 411593, +, 2424, 213145; cds-WP _002884808.1, NZ_KK737549.1, 411642, 412508, -, 867, 414; cds-WP _002884807.1, NZ_KK737549.1, 412521, 413018, -, 498, 445; cds-WP _032428466.1, NZ_KK737549.1, 413206, 414129, -, 924, 235037; cds-WP_002884747.1, NZ_KK737549.1, 414244, 415533, -, 1290, 1109869; cds-WP _002884743.1, NZ_KK737549.1, 415617, 416726, -, 1110, 426756; cds-WP _002884740.1,
NZ_KK737549.1, 417097, 418287, +, 1191, 1983767; cds-WP_002884726.1, NZ_KK737549.1, 418405, 419949, +, 1545, 867090; cds-WP_002884725.1, NZ_KK737549.1, 419964, 420854, +, 891, 53461; cds-WP_002884721.1, NZ_KK737549.1, 421046, 421456, -, 411, 100; cds-WP_013815099.1-65, NZ_KK737549.1, 421813, 422781, -, 969, 5609; cds-WP_021441021.1, NZ_KK737549.1, 422896, 424173, +, 1278, 0; cds-AF52_RS04955, NZ_KK737549.1, 424257, 424484, +, 228, 10; cds-WP_013815099.1-66, NZ_KK737549.1, 424540, 425508, -, 969, 5728; cds-AF52_RS04945, NZ_KK737549.1, 425541, 425747, -, 207, 0; cds-WP_004177855.1, NZ_KK737549.1, 425768, 426088, -, 321, 0; cds-AF52_RS04935, NZ_KK737549.1, 426081, 426602, -, 522, 0; cds-WP_013815099.1-67, NZ_KK737549.1, 426636, 427604, +, 969, 5057; cds-AF52_RS0124460, NZ_KK737549.1, 427655, 427843, +, 189, 25; cds-WP_004177856.1, NZ_KK737549.1, 427951, 428460, +, 510, 89; cds-WP_019725463.1, NZ_KK737549.1, 428469, 428924, +, 456, 853; cds-WP_002884631.1, NZ_KK737549.1, 428974, 430623, -, 1650, 176176; cds-WP_004146591.1, NZ_KK737549.1, 431388, 432737, +, 1350, 388; cds-WP_002884627.1, NZ_KK737549.1, 432876, 433805, +, 930, 0; cds-WP_002884625.1, NZ_KK737549.1, 433922, 434194, +, 273, 817; cds-WP _002884619.1, NZ_KK737549.1, 434191, 435066, -, 876, 32963; cds-WP _002884616.1, NZ_KK737549.1, 435416, 436363, +, 948, 1382; cds-WP_032430268.1, NZ_KK737549.1, 436434, 437237, +, 804, 0; cds-WP _021441018.1, NZ_KK737549.1, 437247, 437654, +, 408, 0; cds-WP_032430269.1, NZ_KK737549.1, 437654, 438148, +, 495, 0; cds-WP_002884608.1, NZ_KK737549.1, 438214, 439014, +, 801, 0; cds-WP_032428464.1, NZ_KK737549.1, 439026, 439850, +, 825, 0; cds-WP _021441016.1, NZ_KK737549.1, 439923, 441155, +, 1233, 0; cds-WP_002884583.1, NZ_KK737549.1, 441148, 441957, +, 810, 1341; cds-WP_004177864.1, NZ_KK737549.1, 442045, 443676, -, 1632, 3; cds-WP _015959249.1, NZ_KK737549.1, 443806, 447489, -, 3684, 65187; cds-WP_002884578.1, NZ_KK737549.1, 447684, 448514, +, 831, 17; cds-WP_002884575.1, NZ_KK737549.1, 448564, 450348, -, 1785, 1298; cds-WP _004192658.1, NZ_KK737549.1, 450451, 451755, -, 1305, 0; cds-WP_002884559.1, NZ_KK737549.1, 451835, 453436, -, 1602, 5; cds-WP_004146576.1, NZ_KK737549.1, 453697, 454626, -, 930, 1; cds-WP_004178258.1, NZ_KK737549.1, 454783, 455244, +, 462,456; cds-WP _032425377.1, NZ_KK737549.1, 462509, 464098, +, 1590, 81404; cds-WP _002884357.1, NZ_KK737549.1, 464114, 465409, +, 1296, 199; cds-WP_032430271.1, NZ_KK737549.1, 465399, 466730, -, 1332, 36; cds-WP_021441012.1, NZ_KK737549.1, 466717, 468111, -, 1395, 0; cds-WP_002884346.1, NZ_KK737549.1, 468371, 468808, +, 438, 0; cds-WP_002884344.1, NZ_KK737549.1, 468812, 469507, -, 696, 11586; cds-WP _002884342.1, NZ_KK737549.1, 469520, 469792, -, 273, 143539; cds-WP _002884331.1, NZ_KK737549.1, 469979, 470569, -, 591, 76038; cds-WP _009485014.1, NZ_KK737549.1, 470612, 471283, -, 672, 6953; cds-WP_004178235.1, NZ_KK737549.1, 471296, 472360, -, 1065, 7997; cds-WP_002884327.1, NZ_KK737549.1, 472401, 473174, -, 774, 2239; cds-WP _002884324.1, NZ_KK737549.1, 473268, 473765, +, 498, 13616; cds-WP _004178230.1, NZ_KK737549.1, 474028, 475923, +, 1896, 1684; cds-WP_004152310.1, NZ_KK737549.1, 475923, 476558, +, 636, 36; cds-WP_004177871.1, NZ_KK737549.1, 476551, 477306, +, 756, 0; cds-WP _002884183.1, NZ_KK737549.1, 477290, 477490, +, 201, 289; cds-WP _004146302.1, NZ_KK737549.1, 477492, 478262, +, 771, 427; cds-WP_004212852.1, NZ_KK737549.1, 478259, 479392, +, 1134, 37800; cds-WP _004178221.1, NZ_KK737549.1, 479709, 481241, +, 1533, 722; cds-WP_0028841501, NZ_KK737549.1, 481414, 481719, +, 306, 16197; cds-WP _004212847.1, NZ_KK737549.1, 481723, 482040, +, 318, 72919; cds-WP_004192681.1, NZ_KK737549.1, 482083, 483423, -, 1341, 67969; cds-WP_002884146.1, NZ_KK737549.1, 483824, 488047, -, 4224, 2351100; cds-WP _004901914.1, NZ_KK737549.1, 488124, 492152, -, 4029, 2263607; cds-WP _002884142.1, NZ_KK737549.1, 492477, 492842, -, 366, 836570; cds-WP_001207203.1, NZ_KK737549.1, 492909, 493406, -, 498, 1552139; cds-WP_002884036.1, NZ_KK737549.1, 493820, 494524, -, 705, 721300; cds-WP _002884034.1, NZ_KK737549.1, 494528, 494956, -, 429, 484358; cds-WP_001287521.1, NZ_KK737549.1, 495113, 495658, -, 546, 80030; cds-WP _002883772.1, NZ_KK737549.1, 495660, 496043, -, 384, 1878; cds-WP _004174069.1-2, NZ_KK737549.1, 496380, 497564, -, 1185, 3484655; cds-WP_002883524.1, NZ_KK737549.1, 498550, 499500, +, 951, 13776; cds-WP_002883521.1, NZ_KK737549.1, 499526, 500488, -, 963, 11366; cds-WP_032430275.1, NZ_KK737549.1, 500485, 501513, -, 1029, 905; cds-WP _002883453.1, NZ_KK737549.1, 509001, 509534, -, 534, 365; cds-WP_002883450.1, NZ_KK737549.1, 509546, 510997, -, 1452, 346; cds-WP_002883449.1, NZ_KK737549.1, 511036, 511650, -, 615, 835; cds-WP_032430276.1, NZ_KK737549.1, 511650, 512981, -, 1332, 26618; cds-WP_032430277.1, NZ_KK737549.1, 513173, 515362, +, 2190, 341493; cds-WP_004150456.1, NZ_KK737549.1, 515372, 516535, +, 1164, 30762; cds-WP _002883448.1, NZ_KK737549.1, 516642, 517343, -, 702, 53616; cds-WP_004146456.1, NZ_KK737549.1, 517393, 518868, -, 1476, 72778; cds-WP _002883430.1, NZ_KK737549.1, 519047, 519535, +, 489, 7728; cds-WP _004146458.1, NZ_KK737549.1, 519532, 520335, -, 804, 93; cds-WP_002883427.1, NZ_KK737549.1, 520368, 521147, -, 780, 3656; cds-WP_002883426.1, NZ_KK737549.1, 521150, 521686, -, 537, 37996; cds-WP _002883425.1, NZ_KK737549.1, 521690, 521941, -, 252, 24160; cds-WP _004177924.1, NZ_KK737549.1, 522071, 523711, -, 1641, 14453; cds-WP _021441002.1, NZ_KK737549.1, 523708, 524313, -, 606, 13216; cds-WP _002883421.1, NZ_KK737549.1, 524327, 525082, -, 756, 25014; cds-WP _021441001.1, NZ_KK737549.1, 525153, 526601, -, 1449, 13788; cds-WP _004181652.1, NZ_KK737549.1, 526737, 527498, -, 762, 21267; cds-WP_002883417.1, NZ_KK737549.1, 527771, 528583, +, 813, 3555; cds-WP_02144100.1, NZ_KK737549.1, 528642, 530906, -, 2265, 0; cds-WP _002883415.1, NZ_KK737549.1, 531154, 532107, +, 954, 8; cds-WP_021440999.1, NZ_KK737549.1, 531995, 532894, -, 900, 9794; cds-WP_004146466.1, NZ_KK737549.1, 532983, 533783, -, 801, 15701; cds-WP_002883412.1, NZ_KK737549.1, 533828, 534820, -, 993, 2295; cds-WP_002883411.1, NZ_KK737549.1, 534930, 535550, +, 621, 1337; cds-WP _021440998.1, NZ_KK737549.1, 535713, 536633, +, 921, 4; cds-WP _021440997.1, NZ_KK737549.1, 536679, 537299, -, 621, 418; cds-WP_032430279.1, NZ_KK737549.1, 537361, 539187, -, 1827, 1679; cds-WP_002883408.1, NZ_KK737549.1, 539255, 540115, -, 861, 2036; cds-WP_002883404.1, NZ_KK737549.1, 540268, 540732, +, 465, 2169; cds-WP _002883402.1, NZ_KK737549.1, 540781, 541674, +, 894, 5427; cds-WP_002883400.1, NZ_KK737549.1, 541718, 542668, -, 951, 3825; cds-WP _002883398.1, NZ_KK737549.1, 543024, 545186, -, 2163, 41372; cds-WP_032430280.1, NZ_KK737549.1, 545250, 545966, -, 717, 5980; cds-WP _002883396.1, NZ_KK737549.1, 545966, 546868, -, 903, 2178; cds-WP_002883395.1, NZ_KK737549.1, 546865, 547572, -, 708, 1482; cds-WP _002883394.1, NZ_KK737549.1, 547569, 548393, -, 825, 18221; cds-WP_002883392.1, NZ_KK737549.1, 548429, 548632, -, 204, 8809; cds-WP_002883389.1, NZ_KK737549.1, 548779, 549099, +, 321, 344; cds-WP_002883383.1, NZ_KK737549.1, 549233, 551785, -, 2553, 39299; cds-WP_021440994.1, NZ_KK737549.1, 552143, 553084, +, 942, 614; cds-WP_012737128.1, NZ_KK737549.1, 553081, 553821, +, 741, 18009; cds-WP _004150429.1, NZ_KK737549.1, 553843, 555024, +, 1182, 24726; cds-WP_004150428.1, NZ_KK737549.1, 555028, 556224, +, 1197, 53883; cds-WP _002883322.1, NZ_KK737549.1, 557224, 558609, -, 1386, 16663; cds-WP _004186366.1, NZ_KK737549.1, 558815, 559555, -, 741, 2; cds-WP _032428460.1, NZ_KK737549.1, 559567, 560913, -, 1347, 16989; cds-WP_021440992.1, NZ_KK737549.1, 560910, 561986, -, 1077, 23520; cds-WP_002883312.1, NZ_KK737549.1, 561983, 563233, -, 1251, 18693; cds-WP _002883310.1, NZ_KK737549.1, 563235, 564365, -, 1131, 15077; cds-WP_004146507.1, NZ_KK737549.1, 564371, 565045, -, 675, 14522; cds-WP_002883307.1, NZ_KK737549.1, 565023, 565904, -, 882, 527; cds-WP_002883303.1, NZ_KK737549.1, 565923, 566990, -, 1068, 17459; cds-WP_021440991.1, NZ_KK737549.1, 566987, 568249, -, 1263, 41483; cds-WP_002883297.1, NZ_KK737549.1, 568246, 569376, -, 1131, 19217; cds-WP_002883296.1, NZ_KK737549.1, 569433, 570479, -, 1047, 1851; cds-WP_002883295.1, NZ_KK737549.1, 570493, 571596, -, 1104, 1523; cds-WP _002883293.1, NZ_KK737549.1, 571837, 573096, -, 1260, 491339; cds-WP _002883224.1, NZ_KK737549.1, 573427, 573756, -, 330, 102411; cds-WP_002883222.1, NZ_KK737549.1, 574098, 575363, +, 1266, 109357; cds-WP_072271788.1, NZ_KK737549.1, 575482, 576990, +, 1509, 7107; cds-WP _002883211.1, NZ_KK737549.1, 577010, 577936, +, 927, 2834; cds-WP_004181642.1, NZ_KK737549.1, 578067, 579065, +, 999, 1; cds-WP_002883185.1, NZ_KK737549.1, 579128, 579850, +, 723, 6630; cds-WP_032430283.1, NZ_KK737549.1, 579855, 581876, -, 2022, 8246; cds-WP _002883181.1, NZ_KK737549.1, 581989, 582270, +, 282, 2; cds-WP _002883180.1, NZ_KK737549.1, 582322, 582603, +, 282, 2853; cds-WP_002883178.1, NZ_KK737549.1, 582807, 584282, -, 1476, 58587; cds-WP_002883176.1, NZ_KK737549.1, 584435, 585325, +, 891, 14726; cds-WP _002883174.1, NZ_KK737549.1, 585322, 586866, -, 1545, 23664; cds-WP_002883173.1, NZ_KK737549.1, 586869, 588719, -, 1851, 13594; cds-WP _032430284.1, NZ_KK737549.1, 588780, 589709, -, 930, 3563; cds-WP_002883170.1, NZ_KK737549.1, 589726, 589983, -, 258, 0; cds-WP_012737117.1, NZ_KK737549.1, 589980, 591626, -, 1647, 6; cds-WP_001311244.1, NZ_KK737549.1, 591767, 591865, -, 99, 0; cds-WP_032430285.1, NZ_KK737549.1, 592220, 593740, +, 1521, 398; cds-WP_004146520.1, NZ_KK737549.1, 593766, 594104, -, 339, 104; cds-WP _004177958.1, NZ_KK737549.1, 594223, 595044, +, 822, 8379; cds-WP _032430287.1, NZ_KK737549.1, 603456, 604307, -, 852, 13533; cds-WP _004146288.1, NZ_KK737549.1, 604252, 606090, -, 1839, 29922; cds-WP_002883023.1, NZ_KK737549.1, 606457, 607557, +, 1101, 16111; cds-WP_002883021.1, NZ_KK737549.1, 607609, 607968, -, 360, 27802; cds-WP _004151856.1, NZ_KK737549.1, 607984, 608619, -, 636, 7400; cds-WP _002883016.1, NZ_KK737549.1, 608816, 610216, +, 1401, 227632; cds-WP_002883015.1, NZ_KK737549.1, 610199, 611116, -, 918, 27965; cds-WP_032430289.1, NZ_KK737549.1, 611375, 612748, -, 1374, 741; cds-WP_004187843.1, NZ_KK737549.1, 612848, 613624, -, 777, 21; cds-WP_004181637.1, NZ_KK737549.1, 613631, 614635, -, 1005, 646; cds-WP_019705783.1, NZ_KK737549.1, 614749, 615900, +, 1152, 2567; cds-WP_004181636.1, NZ_KK737549.1, 616159, 618810, +, 2652, 9828; cds-WP_021440981.1, NZ_KK737549.1, 618854, 619504, -, 651, 0; cds-WP _004220885.1, NZ_KK737549.1, 619529, 620515, +, 987, 40; cds-WP _004177977.1, NZ_KK737549.1, 620721, 621383, +, 663, 0; cds-WP _004146274.1, NZ_KK737549.1, 621438, 622541, +, 1104, 1556; cds-WP_004177983.1, NZ_KK737549.1, 622605, 623486, -, 882, 0; cds-WP _021440979.1, NZ_KK737549.1, 623630, 624517, -, 888, 0; cds-WP_021440978.1, NZ_KK737549.1, 624746, 625654, -, 909, 27360; cds-WP_021440977.1, NZ_KK737549.1, 625746, 626117, +, 372, 0; cds-WP _032428457.1, NZ_KK737549.1, 626155, 627711, -, 1557, 0; cds-WP_013815099.1-68, NZ_KK737549.1, 627780, 628748, +, 969, 4589; cds-WP _004181629.1, NZ_KK737549.1, 628916, 630052, +, 1137, 13; cds-WP _032430291.1, NZ_KK737549.1, 630027, 632459, -, 2433, 121; cds-WP_004150388.1, NZ_KK737549.1, 632462, 633622, -, 1161, 6333; cds-WP_002882924.1, NZ_KK737549.1, 633890, 634207, +, 318, 4261; cds-WP_002882922.1, NZ_KK737549.1, 634309, 634521, -, 213, 28424; cds-WP _025710954.1, NZ_KK737549.1, 634774, 636969, +, 2196, 673; cds-WP _002882921.1, NZ_KK737549.1, 637120, 638148, +, 1029, 44908; cds-WP _071600598.1, NZ_KK737549.1, 638242, 639210, +, 969, 28626; cds-WP_002882918.1, NZ_KK737549.1, 639302, 639832, +, 531, 22458; cds-WP_002882917.1, NZ_KK737549.1, 639842, 641176, +, 1335, 143779; cds-WP _032430292.1, NZ_KK737549.1, 641246, 642166, +, 921, 14172; cds-WP _032430293.1, NZ_KK737549.1, 642259, 642744, +, 486, 3901; cds-WP _002882911.1, NZ_KK737549.1, 642806, 643768, -, 963, 39385; cds-WP_002882907.1, NZ_KK737549.1, 643965, 644867, -,903,1081; cds-WP_002882903.1, NZ_KK737549.1, 645026, 645529, -, 504, 13728; cds-WP_002882901.1, NZ_KK737549.1, 645679, 646377, +, 699, 27846; cds-WP _032430294.1, NZ_KK737549.1, 646374, 647747, +, 1374, 35592; cds-WP_002882896.1, NZ_KK737549.1, 647829, 648503, -, 675, 162; cds-WP_002882894.1, NZ_KK737549.1, 648576, 649196, -, 621, 457098; cds-WP_032430295.1, NZ_KK737549.1, 649486, 650520, +, 1035, 866; cds-WP _002882891.1, NZ_KK737549.1, 650553, 651398, -, 846, 559; cds-WP _002882888.1,
NZ_KK737549.1, 651472, 652308, -, 837, 0; cds-WP_002882885.1, NZ_KK737549.1, 652619, 654085, +, 1467, 0; cds-WP _004150375.1, NZ_KK737549.1, 654082, 655341, +, 1260, 0; cds-WP_004187188.1, NZ_KK737549.1, 655491, 656321, +, 831, 2; cds-WP _002882880.1, NZ_KK737549.1, 656403, 657389, +, 987, 1; cds-WP_021440966.1, NZ_KK737549.1, 657535, 659046, +, 1512, 12; cds-WP _004181621.1, NZ_KK737549.1, 659043, 660050, +, 1008, 0; cds-WP _032430296.1, NZ_KK737549.1, 660047, 661051, +, 1005, 0; cds-WP_002882871.1, NZ_KK737549.1, 661141, 662289, +, 1149, 1; cds-WP _021440964.1, NZ_KK737549.1, 662286, 662600, +, 315, 0; cds-WP_004210464.1, NZ_KK737549.1, 662638, 663903, -, 1266, 1650; cds-WP_004178018.1, NZ_KK737549.1, 663945, 666068, -, 2124, 22086; cds-WP_021440963.1, NZ_KK737549.1, 666184, 667179, -, 996, 4; cds-WP_002882865.1, NZ_KK737549.1, 667267, 667815, -, 549, 0; cds-WP_032430297.1, NZ_KK737549.1, 667915, 668571, +, 657, 0; cds-WP _004146241.1, NZ_KK737549.1, 668571, 668894, +, 324, 0; cds-WP _002882830.1, NZ_KK737549.1, 668935, 669147, -, 213, 0; cds-WP_032430298.1, NZ_KK737549.1, 669276, 670112, -, 837, 6; cds-WP _085921749.1, NZ_KK737549.1, 670269, 673319, +, 3051, 1008492; cds-WP_002882822.1, NZ_KK737549.1, 673332, 674234, +, 903, 21748; cds-WP _032430299.1, NZ_KK737549.1, 674231, 674866, +, 636, 116385; cds-WP _004150358.1, NZ_KK737549.1, 674863, 675792, +, 930, 136306; cds-WP _019705794.1, NZ_KK737549.1, 675966, 676349, -, 384, 297; cds-WP_004150355.1, NZ_KK737549.1, 676349, 676591, -, 243, 79; cds-WP _032430300.1, NZ_KK737549.1, 676796, 677713, +, 918, 7635; cds-WP _004178031.1, NZ_KK737549.1, 677728, 678669, -, 942, 468; cds-WP _002882809.1, NZ_KK737549.1, 678714, 679151, -, 438, 7; cds-WP _004146232.1, NZ_KK737549.1, 679148, 680008, -, 861, 245; cds-WP_004151865.1, NZ_KK737549.1, 680002, 680601, -, 600, 1879; cds-WP_002882755.1, NZ_KK737549.1, 680737, 682560, -, 1824, 210469; cds-WP _002882753.1, NZ_KK737549.1, 682939, 684348, +, 1410, 111458; cds-WP_004146229.1, NZ_KK737549.1, 684537, 685586, +, 1050, 11; cds-WP_002882749.1, NZ_KK737549.1, 685595, 687004, +, 1410, 25824; cds-WP_004152951.1, NZ_KK737549.1, 687115, 687225, +, 111, 1576; cds-WP_002882745.1, NZ_KK737549.1, 687261, 688634, -, 1374, 3295; cds-WP_002882743.1, NZ_KK737549.1, 688823, 689329, -, 507, 1545; cds-WP _002882734.1, NZ_KK737549.1, 689913, 690545, +, 633, 15890; cds-WP _002882729.1, NZ_KK737549.1, 690893, 693685, -, 2793, 14370; cds-WP_004150336.1, NZ_KK737549.1, 694082, 694981, +, 900, 1990; cds-WP _004146224.1, NZ_KK737549.1, 695030, 695653, -, 624, 26320; cds-WP_004187940.1, NZ_KK737549.1, 695681, 696667, -, 987, 16802; cds-WP _002882612.1, NZ_KK737549.1, 696743, 697012, -, 270, 228; cds-WP _004178039.1, NZ_KK737549.1, 697084, 697665, +, 582, 2; cds-WP_002882596.1, NZ_KK737549.1, 697662, 698168, +, 507, 7; cds-WP_004150973.1, NZ_KK737550.1, 3504, 6077, -, 2574, 72041; cds-WP_021440571.1, NZ_KK737550.1, 6207, 6938, -, 732, 18103; cds-WP _032428369.1, NZ_KK737550.1, 6935, 7915, -, 981, 7730; cds-WP _004145664.1, NZ_KK737550.1, 8047, 8784, +, 738, 79428; cds-WP_002914111.1, NZ_KK737550.1, 9055, 9390, +, 336, 29053; cds-WP_100250063.1, NZ_KK737550.1, 9497, 9544, +, 48, 1; cds-WP _021440574.1, NZ_KK737550.1, 9645, 10805, +, 1161, 8133; cds-WP _021440575.1, NZ_KK737550.1, 10802, 11674, -, 873, 49; cds-WP _014907099.1, NZ_KK737550.1, 11737, 12858, -, 1122, 30009; cds-WP _002914114.1, NZ_KK737550.1, 12868, 13938, -, 1071, 481; cds-WP_004174804.1, NZ_KK737550.1, 14281, 14790, +, 510, 14; cds-WP _002914116.1, NZ_KK737550.1, 14783, 16006, +, 1224, 331; cds-WP _004145671.1, NZ_KK737550.1, 16020, 16502, +, 483, 4060; cds-WP _023285466.1, NZ_KK737550.1, 16511, 17881, -, 1371, 392; cds-WP_002914119.1, NZ_KK737550.1, 17938, 18396, -, 459, 77; cds-WP_002914145.1, NZ_KK737550.1, 18516, 18863, -, 348, 209740; cds-WP _002914147.1, NZ_KK737550.1, 18903, 19670, -, 768, 1111933; cds-WP_004150977.1, NZ_KK737550.1, 19702, 20250, -, 549, 737611; cds-WP _002914149.1, NZ_KK737550.1, 20269, 20517, -, 249, 186675; cds-WP _002914152.1, NZ_KK737550.1, 20777, 22141, -, 1365, 54018; cds-WP _002914153.1, NZ_KK737550.1, 22305, 23096, +, 792, 1840; cds-WP _004149392.1, NZ_KK737550.1, 23116, 24402, +, 1287, 207; cds-WP _004174800.1, NZ_KK737550.1, 24523, 25113, -, 591, 37358; cds-WP_002914158.1, NZ_KK737550.1, 25238, 26116, +, 879, 12395; cds-WP _004174799.1, NZ_KK737550.1, 26203, 27864, +, 1662, 276; cds-WP _002914160.1, NZ_KK737550.1, 28012, 28353, +, 342, 17238; cds-WP _004145682.1, NZ_KK737550.1, 28420, 28710, -, 291, 3391; cds-WP _015875096.1, NZ_KK737550.1, 28700, 29176, -, 477, 2511; cds-WP_002914164.1, NZ_KK737550.1, 29287, 29769, +, 483, 16221; cds-WP _032430315.1, NZ_KK737550.1, 30546, 30794, +, 249, 2172; cds-WP _032430316.1, NZ_KK737550.1, 31673, 32251, -, 579, 0; cds-WP _032430369.1, NZ_KK737550.1, 32977, 34431, +, 1455, 2874; cds-WP _032430317.1, NZ_KK737550.1, 34432, 35181, +, 750, 3959; cds-WP _032430318.1, NZ_KK737550.1, 35321, 36064, -, 744, 0; cds-WP_032430320.1, NZ_KK737550.1, 36079, 37839, -, 1761, 3; cds-AF52_RS03525, NZ_KK737550.1, 37916, 38458, -, 543, 0; cds-WP_013815099.1-69, NZ_KK737550.1, 38493, 39461, +, 969, 5161; cds-AF52_RS0124535, NZ_KK737550.1, 39512, 39685, +, 174, 41; cds-WP _002914289.1, NZ_KK737550.1, 39771, 40220, -, 450, 8401; cds-WP _004217497.1, NZ_KK737550.1, 40931, 41350, +, 420, 10318; cds-WP_002914284.1, NZ_KK737550.1, 41423, 41602, -, 180, 13312; cds-AF52_RS03500, NZ_KK737550.1, 41808, 42377, +, 570, 0; cds-WP_013815099.1-70, NZ_KK737550.1, 42433, 43401, -, 969, 5276; cds-AF52_RS03490, NZ_KK737550.1, 43435, 43776, +, 342, 56; cds-WP _021440595.1, NZ_KK737550.1, 43757, 44302, -, 546, 7; cds-WP _002914277.1, NZ_KK737550.1, 44310, 44609, -, 300, 238; cds-WP_004212889.1, NZ_KK737550.1, 44685, 45290, -, 606, 0; cds-WP _021440594.1, NZ_KK737550.1, 45394, 46302, +, 909, 6418; cds-WP_021440593.1, NZ_KK737550.1, 46385, 48172, -, 1788, 15537; cds-WP_004174747.1, NZ_KK737550.1, 48436, 49956, +, 1521, 3047; cds-WP_013815099.1-71, NZ_KK737550.1, 50334, 51302, +, 969, 4313; cds-WP _032430322.1, NZ_KK737550.1, 51339, 51674, +, 336, 25; cds-WP _021440597.1, NZ_KK737550.1, 51818, 53170, -, 1353, 3; cds-WP_004151037.1, NZ_KK737550.1, 53259, 53690, +, 432, 676; cds-WP 002914320.1, NZ_KK737550.1, 53874, 54119, +, 246, 38; cds-WP_002914321.1, NZ_KK737550.1, 54116, 54526, +, 411, 10; cds-WP _032428382.1, NZ_KK737550.1, 54499, 56640, +, 2142, 34; cds-WP _021440599.1, NZ_KK737550.1, 56651, 57613, +, 963, 5; cds-WP_002914328.1, NZ_KK737550.1, 57970, 59172, +, 1203, 0; cds-WP_021312381.1, NZ_KK737550.1, 59165, 60232, +, 1068, 0; cds-WP _002914330.1, NZ_KK737550.1, 60311, 61309, +, 999, 2; cds-WP_002914333.1, NZ_KK737550.1, 61487, 62233, +, 747, 584; cds-WP_021440601.1, NZ_KK737550.1, 62223, 62558, +, 336, 75; cds-WP_002914337.1, NZ_KK737550.1, 62649, 63179, +, 531, 59733; cds-WP_002914339.1, NZ_KK737550.1, 63305, 64477, +, 1173, 34339; cds-WP_002914342.1, NZ_KK737550.1, 64493, 66031, +, 1539, 129; cds-WP _004185353.1, NZ_KK737550.1, 66168, 67844, +, 1677, 79; cds-WP_004149452.1, NZ_KK737550.1, 67823, 68590, -, 768, 7974; cds-WP_002914351.1, NZ_KK737550.1, 68643, 69158, -, 516, 14017; cds-WP_002914353.1, NZ_KK737550.1, 69315, 70871, -, 1557, 153982; cds-WP_002914355.1, NZ_KK737550.1, 70938, 71366, -, 429, 746; cds-WP _004174701.1, NZ_KK737550.1, 71363, 71929, -, 567, 2794; cds-WP_002914359.1, NZ_KK737550.1, 72055, 72369, -, 315, 4223; cds-WP_000906486.1, NZ_KK737550.1, 73958, 74143, -, 186, 4496; cds-WP _004174700.1, NZ_KK737550.1, 74507, 77134, -, 2628, 416693; cds-WP_004213244.1, NZ_KK737550.1, 77385, 77885, -, 501, 22; cds-WP_002914769.1, NZ_KK737550.1, 77953, 79011, -, 1059, 170494; cds-WP_002914771.1, NZ_KK737550.1, 79102, 79599, -, 498, 118; cds-WP_032428383.1, NZ_KK737550.1, 79739, 80617, -, 879, 0; cds-WP_004149458.1, NZ_KK737550.1, 80625, 81488, -, 864, 0; cds-AF52_RS0124565, NZ_KK737550.1, 81485, 82195, -, 711, 1; cds-WP_004174694.1, NZ_KK737550.1, 82534, 83613, -, 1080, 571; cds-WP _002914812.1, NZ_KK737550.1, 83890, 84453, +, 564, 20114; cds-WP _021440607.1, NZ_KK737550.1, 84450, 85430, +, 981, 38665; cds-WP_004174693.1, NZ_KK737550.1, 85444, 85806, +, 363, 1209; cds-WP_002914815.1, NZ_KK737550.1, 85819, 86598, +, 780, 0; cds-WP_021440608.1, NZ_KK737550.1, 86756, 87115, +, 360, 0; cds-WP_002914817.1, NZ_KK737550.1, 87182, 87955, +, 774, 2042; cds-WP _004174691.1, NZ_KK737550.1, 87948, 88913, +, 966, 3190; cds-WP_004890240.1, NZ_KK737550.1, 88876, 90426, -, 1551, 107; cds-WP_004145759.1, NZ_KK737550.1, 90614, 92062, +, 1449, 0; cds-WP_021440610.1, NZ_KK737550.1, 92059, 93192, +, 1134, 0; cds-WP _004181028.1, NZ_KK737550.1, 93289, 94302, -, 1014, 465; cds-WP _032430324.1, NZ_KK737550.1, 94299, 96515, -, 2217, 51; cds-WP_002914875.1, NZ_KK737550.1, 96524, 97051, -, 528, 1922; cds-WP _004174685.1, NZ_KK737550.1, 97188, 98201, -, 1014, 302; cds-WP _032430325.1, NZ_KK737550.1, 98469, 99923, +, 1455, 356; cds-WP _004181034.1, NZ_KK737550.1, 99938, 101362, +, 1425, 53015; cds-WP _002914883.1, NZ_KK737550.1, 101544, 101852, +, 309, 4; cds-WP_032430326.1, NZ_KK737550.1, 101849, 102319, -, 471, 5; cds-WP_002914887.1, NZ_KK737550.1, 102312, 102722, -, 411, 0; cds-WP _002914891.1, NZ_KK737550.1, 102719, 103486, -, 768, 8; cds-WP_002914893.1, NZ_KK737550.1, 103486, 104028, -, 543, 0; cds-WP_021440614.1, NZ_KK737550.1, 104038, 105747, -, 1710, 0; cds-WP_032428387.1, NZ_KK737550.1, 105764, 106687, -, 924, 0; cds-WP_004890213.1, NZ_KK737550.1, 106690, 108516, -, 1827, 0; cds-WP _004151047.1, NZ_KK737550.1, 108513, 109121, -, 609, 0; cds-WP_002914925.1, NZ_KK737550.1, 109204, 109653, -, 450, 0; cds-WP _002914928.1, NZ_KK737550.1, 109863, 110207, +, 345, 0; cds-WP_004181043.1, NZ_KK737550.1, 110211, 111083, +, 873, 0; cds-WP_014907033.1, NZ_KK737550.1, 111074, 111346, +, 273, 0; cds-WP_004145781.1, NZ _KK737550.1, 111346, 112467, +, 1122, 0; cds-WP _032428388.1, NZ_KK737550.1, 112464, 113474, +, 1011, 0; cds-WP_032428389.1, NZ_KK737550.1, 113711, 115783, +, 2073, 515; cds-WP_002914970.1, NZ_KK737550.1, 115837, 117237, -, 1401, 1116; cds-WP_021440618.1, NZ_KK737550.1, 117288, 119480, -, 2193, 2644; cds-AF52_RS03110, NZ_KK737550.1, 119493, 120829, -, 1337, 6193; cds-WP_032430327.1, NZ_KK737550.1, 121092, 122363, -, 1272, 3; cds-WP _002914979.1, NZ_KK737550.1, 122541, 122855, +, 315, 0; cds-WP_002914981.1, NZ_KK737550.1, 122865, 124187, +, 1323, 0; cds-WP _032430328.1, NZ_KK737550.1, 124211, 124525, +, 315, 7; cds-WP_004151053.1, NZ_KK737550.1, 124576, 125490, +, 915, 7; cds-WP_004181055.1, NZ_KK737550.1, 125547, 126509, +, 963, 7336; cds-WP _004174672.1, NZ_KK737550.1, 126688, 127605, +, 918, 45; cds-WP_021440623.1, NZ_KK737550.1, 127602, 128423, +, 822, 8; cds-WP_004185394.1, NZ_KK737550.1, 128420, 129268, +, 849, 0; cds-WP_002915035.1, NZ _KK737550.1, 129262, 130116, +, 855, 2; cds-WP _004193741.1, NZ_KK737550.1, 130183, 130524, -, 342, 0; cds-WP_032430329.1, NZ_KK737550.1, 130591, 131478, -, 888, 1; cds-WP _002915041.1, NZ_KK737550.1, 131663, 132604, +, 942, 0; cds-WP_032430330.1, NZ_KK737550.1, 132640, 134349, +, 1710, 0; cds-WP_021440627.1, NZ_KK737550.1, 134389, 134682, -, 294, 1; cds-WP_021440628.1, NZ_KK737550.1, 134898, 137261, +, 2364, 121; cds-WP _032428391.1, NZ_KK737550.1, 137333, 138364, +, 1032, 12; cds-WP _004181072.1, NZ_KK737550.1, 138361, 139179, +, 819, 0; cds-WP_032428392.1, NZ_KK737550.1, 139179, 140174, +, 996, 11; cds-WP_021440630.1, NZ_KK737550.1, 140167, 140946, +, 780, 4991; cds-WP_032430331.1, NZ_KK737550.1, 141100, 143661, +, 2562, 2207; cds-WP _021440632.1, NZ_KK737550.1, 143781, 143933, -, 153, 0; cds-WP _002915098.1, NZ_KK737550.1, 144272, 144508, -, 237, 0; cds-WP _004151060.1, NZ_KK737550.1, 144519, 145946, -, 1428, 12; cds-WP_002915099.1, NZ_KK737550.1, 145946, 146539, -, 594, 0; cds-WP_002915102.1, NZ_KK737550.1, 146713, 147120, +, 408, 18; cds-WP _032430332.1, NZ _KK737550.1, 147129, 148016, -, 888, 42; cds-WP _021440634.1, NZ_KK737550.1, 148125, 149327, +, 1203, 223; cds-WP _021440635.1, NZ_KK737550.1, 149324, 149701, +, 378, 0; cds-WP _002915106.1, NZ_KK737550.1, 149754, 150746, -, 993, 110084; cds-WP_014907019.1, NZ_KK737550.1, 150904, 152034, -, 1131, 254576; cds-WP _002915108.1, NZ_KK737550.1, 152160, 152786, -, 627, 89268; cds-WP _004142750.1, NZ_KK737550.1, 152780, 153541, -, 762, 17550; cds-WP_004185423.1, NZ_KK737550.1, 153522, 154571, -, 1050, 2019; cds-WP_002915113.1, NZ_KK737550.1, 154568, 155047, -, 480, 9453; cds-WP _021440636.1, NZ_KK737550.1, 155047, 155757, -, 711, 274; cds-WP_002915156.1, NZ_KK737550.1, 155777, 156094, -, 318, 12566; cds-WP _002915157.1,
NZ_KK737550.1, 156235, 156561, -, 327, 0; cds-WP_002915158.1, NZ_KK737550.1, 156612, 157217, -, 606, 53547; cds-WP_004181094.1, NZ_KK737550.1, 157217, 158644, -, 1428, 11599; cds-WP_002915160.1, NZ_KK737550.1, 158654, 159562, -, 909, 545; cds-WP_072271746.1, NZ_KK737550.1, 159572, 160978, -, 1407, 17; cds-WP_004174643.1, NZ_KK737550.1, 161225, 162271, +, 1047, 98; cds-WP_002915177.1, NZ_KK737550.1, 162344, 163078, -, 735, 1364; cds-WP _021440639.1, NZ_KK737550.1, 163177, 164889, -, 1713, 87603; cds-WP_004174642.1, NZ_KK737550.1, 164889, 166688, -, 1800, 1090; cds-WP_187079196.1, NZ_KK737550.1, 167005, 167370, +, 366, 10605; cds-WP_021440641.1, NZ_KK737550.1, 167411, 168082, -, 672, 197; cds-WP_032430333.1, NZ_KK737550.1, 168557, 169666, +, 1110, 17; cds-WP_002915213.1, NZ_KK737550.1, 169730, 171028, -, 1299, 1263201; cds-WP_004142808.1, NZ_KK737550.1, 171110, 172747, -, 1638, 221219; cds-WP_002915215.1, NZ_KK737550.1, 173028, 173819, -, 792, 1; cds-WP_002915217.1, NZ_KK737550.1, 173931, 176168, -, 2238, 32423; cds-WP_023301525.1, NZ_KK737550.1, 176290, 177594, -, 1305, 3509; cds-WP_004151066.1, NZ_KK737550.1, 177708, 180458, +, 2751, 6588; cds-WP_004149562.1, NZ_KK737550.1, 180616, 181755, -, 1140,44848; cds-WP_002915223.1, NZ_KK737550.1, 181828, 183168, -, 1341, 37047; cds-WP_032428394.1, NZ_KK737550.1, 183186, 184526, -, 1341, 17358; cds-WP_002915225.1, NZ_KK737550.1, 184529, 185881, -, 1353, 18326; cds-WP_002915235.1, NZ_KK737550.1, 186353, 186796, -, 444, 2096; cds-WP_002915236.1, NZ_KK737550.1, 186801, 187595, -, 795, 4534; cds-WP_002915249.1, NZ_KK737550.1, 187595, 187924, -, 330, 2; cds-WP_002915253.1, NZ_KK737550.1, 188556, 189101, -, 546, 10178; cds-WP_004174633.1, NZ_KK737550.1, 189170, 190015, +, 846, 1107; cds-WP_002915258.1, NZ_KK737550.1, 190127, 191494, +, 1368, 63335; cds-WP_004151070.1, NZ_KK737550.1, 192004, 193293, +, 1290, 1361800; cds-WP_002915259.1, NZ_KK737550.1, 193359, 194726, +, 1368,410917; cds-WP_032430336.1, NZ_KK737550.1, 194865, 195806, +, 942, 6399; cds-WP_002915263.1, NZ_KK737550.1, 195852, 197075, -, 1224, 0; cds-WP_004151071.1, NZ_KK737550.1, 197086, 198507, -, 1422, 368; cds-WP_002915265.1, NZ_KK737550.1, 198701, 199579, -, 879, 0; cds-WP_002915267.1, NZ_KK737550.1, 199576, 200274, -, 699, 0; cds-WP_004174621.1, NZ_KK737550.1, 200271, 201293, -, 1023, 0; cds-WP_032428395.1, NZ_KK737550.1, 201290, 202696, -, 1407, 0; cds-AF52_RS0124590, NZ_KK737550.1, 202770, 204167, -, 1398, 0; cds-WP_021440649.1, NZ_KK737550.1, 204214, 205386, -, 1173, 0; cds-WP_032428396.1, NZ_KK737550.1, 205464, 205580, -, 117, 0; cds-WP_021440650.1, NZ_KK737550.1, 205658, 207601, +, 1944, 0; cds-WP_004142871.1, NZ_KK737550.1, 207698, 208453, +, 756, 197; cds-WP_032430337.1, NZ_KK737550.1, 208512, 209660, -, 1149, 0; cds-WP_002915470.1, NZ_KK737550.1, 209688, 210335, -, 648, 1; cds-WP_004151076.1, NZ_KK737550.1, 210902, 212212, +, 1311, 0; cds-WP_032430338.1, NZ_KK737550.1, 212245, 214020, +, 1776, 0; cds-WP_004181132.1, NZ_KK737550.1, 214128, 215546, +, 1419, 0; cds-WP_002915538.1, NZ_KK737550.1, 215548, 215970, +, 423, 0; cds-WP_004142883.1, NZ_KK737550.1, 216029, 216739, +, 711, 306; cds-WP_002915541.1, NZ_KK737550.1, 216788, 217888, -, 1101, 24237; cds-WP_002915543.1, NZ_KK737550.1, 217881, 218276, -, 396, 11144; cds-WP_002915545.1, NZ_KK737550.1, 218296, 219213, -, 918, 13718; cds-WP _002915549.1, NZ_KK737550.1, 219565, 219789, -, 225, 447; cds-WP_021440652.1, NZ_KK737550.1, 219987, 221192, +, 1206, 20; cds-WP_021440653.1, NZ_KK737550.1, 221192, 221623, +, 432, 6; cds-WP_002915577.1, NZ_KK737550.1, 221666, 222487, -, 822, 19531; cds-WP_002915579.1, NZ_KK737550.1, 222575, 223672, -, 1098, 16983; cds-WP_004900253.1, NZ_KK737550.1, 224055, 225110, -, 1056, 2476; cds-WP_004143921.1, NZ_KK737550.1, 225154, 225699, -, 546, 6; cds-WP_021440655.1, NZ_KK737550.1, 225693, 227216, -, 1524, 6; cds-WP_002915614.1, NZ_KK737550.1, 227213, 228037, -, 825, 3891; cds-WP_004143925.1, NZ_KK737550.1, 228046, 228723, -, 678, 39; cds-WP_002915619.1, NZ_KK737550.1, 228710, 228991, -, 282, 0; cds-WP_004143926.1, NZ_KK737550.1, 228993, 229730, -, 738, 0; cds-WP_004143927.1, NZ_KK737550.1, 229730, 230440, -, 711, 278; cds-WP_016532596.1, NZ_KK737550.1, 230437, 231231, -, 795, 1; cds-WP_002915627.1, NZ_KK737550.1, 231242, 232027, -, 786, 1; cds-WP_004143930.1, NZ_KK737550.1, 232024, 232749, -, 726, 5; cds-WP_021440657.1, NZ_KK737550.1, 232749, 233804, -, 1056, 8; cds-WP_004149602.1, NZ_KK737550.1, 233785, 234558, -, 774, 0; cds-WP_032430341.1, NZ_KK737550.1, 234551, 235120, -, 570, 1813; cds-WP_016947545.1, NZ_KK737550.1, 235110, 235715, -, 606, 0; cds-WP _002915708.1, NZ_KK737550.1, 235709, 236848, -, 1140, 624; cds-WP _021440659.1, NZ_KK737550.1, 236845, 237480, -, 636, 30; cds-WP_002915724.1, NZ_KK737550.1, 237491, 238450, -, 960, 0; cds-WP_004185466.1, NZ_KK737550.1, 238447, 239823, -, 1377, 1; cds-AF52_RS02545, NZ_KK737550.1, 240376, 241203, -, 828, 0; cds-AF52_RS26545, NZ_KK737550.1, 241259, 241436, -, 178, 208; cds-WP_170999280.1, NZ_KK737550.1, 241947, 242236, -, 290, 245; cds-WP_002915737.1, NZ_KK737550.1, 242699, 242983, +, 285, 0; cds-WP_002915739.1, NZ_KK737550.1, 242980, 243792, +, 813, 0; cds-WP_002915744.1, NZ_KK737550.1, 243811, 245475, +, 1665, 0; cds-WP_008806302.1, NZ_KK737550.1, 245486, 246175, +, 690, 0; cds-WP_021440661.1, NZ_KK737550.1, 246190, 246714, +, 525, 0; cds-WP_021440662.1, NZ_KK737550.1, 246727, 248559, +, 1833, 25; cds-WP_004900279.1, NZ_KK737550.1, 248549, 248899, +, 351, 0; cds-WP_002915806.1, NZ_KK737550.1, 248918, 249193, +, 276, 0; cds-WP_002915808.1, NZ_KK737550.1, 249215, 249661, +, 447, 0; cds-WP_002915810.1, NZ_KK737550.1, 249661, 250293, +, 633, 0; cds-WP_021440663.1, NZ_KK737550.1, 250290, 250781, +, 492, 0; cds-WP_021440664.1, NZ_KK737550.1, 250785, 251060, +, 276, 0; cds-WP_004211864.1, NZ_KK737550.1, 251071, 252087, +, 1017, 0; cds-WP_021440665.1, NZ_KK737550.1, 252084, 253472, +, 1389, 0; cds-WP_021440666.1, NZ_KK737550.1, 253484, 254596, +, 1113, 0; cds-WP_023301518.1, NZ_KK737550.1, 254593, 255945, +, 1353, 0; cds-WP_004181171.1, NZ_KK737550.1, 255948, 256502, +, 555, 0; cds-WP_002915850.1, NZ_KK737550.1, 256502, 256852, +, 351, 0; cds-WP_004185483.1, NZ_KK737550.1, 256857, 257297, +, 441, 0; cds-WP_032430342.1, NZ_KK737550.1, 257294, 258508, +, 1215, 2156; cds-WP_032428406.1, NZ_KK737550.1, 258600, 259466, +, 867, 2; cds-WP_004143954.1, NZ_KK737550.1, 259470, 260546, -, 1077, 12952; cds-WP_004174538.1, NZ_KK737550.1, 260564, 261817, -, 1254, 648; cds-AF52_RS02415, NZ_KK737550.1, 262045, 263376, +, 1332, 552; cds-WP_009485668.1, NZ_KK737550.1, 263606, 263926, +, 321, 2; cds-WP_004900323.1, NZ_KK737550.1, 263984, 265828, -, 1845, 1; cds-WP_021440669.1, NZ_KK737550.1, 265825, 269361, -, 3537, 47236; cds-WP_023301515.1, NZ_KK737550.1, 269358, 272243, -, 2886, 39381; cds-WP_004211885.1, NZ_KK737550.1, 272364, 275741, -, 3378, 20440; cds-WP_021440672.1, NZ_KK737550.1, 275754, 276077, -, 324, 0; cds-WP_004181182.1, NZ_KK737550.1, 276062, 276469, -, 408, 5; cds-WP _021440673.1, NZ_KK737550.1, 276466, 277023, -, 558, 0; cds-WP_002915932.1, NZ_KK737550.1, 277020, 277571, -, 552, 0; cds-WP_002915933.1, NZ_KK737550.1, 277757, 278551, -, 795, 6366; cds-WP_002915934.1, NZ_KK737550.1, 278558, 279433, -, 876, 24052; cds-WP_032430343.1, NZ_KK737550.1, 279679, 281925, -, 2247, 27541; cds-WP_002915936.1, NZ_KK737550.1, 281938, 282468, -, 531, 47153; cds-WP_004218186.1, NZ_KK737550.1, 282922, 283065, +, 144, 0; cds-WP_004143967.1, NZ_KK737550.1, 283151, 283846, +, 696, 0; cds-WP_002915973.1, NZ_KK737550.1, 283910, 284623, +, 714, 3156; cds-WP_002915974.1, NZ_KK737550.1, 284747, 284965, +, 219, 21540; cds-WP _032430346.1, NZ_KK737550.1, 285186, 286226, +, 1041, 177; cds-WP_004185495.1, NZ_KK737550.1, 286329, 287522, -, 1194, 472; cds-WP_002915977.1, NZ_KK737550.1, 287515, 289674, -, 2160, 13923; cds-AF52_RS28115, NZ_KK737550.1, 289938, 290202, -, 265, 0; cds-WP_002915981.1, NZ_KK737550.1, 290296, 291312, +, 1017, 3876; cds-WP_021440677.1, NZ_KK737550.1, 291273, 291752, -, 480, 34; cds-WP_032430347.1, NZ_KK737550.1, 291749, 292522, -, 774, 186; cds-WP_004174521.1, NZ_KK737550.1, 292590, 294017, -, 1428, 15142; cds-WP_004174520.1, NZ_KK737550.1, 294025, 295362, -, 1338, 5; cds-WP_021440678.1, NZ_KK737550.1, 295662, 296663, +, 1002, 53852; cds-WP_002915990.1, NZ_KK737550.1, 296656, 297918, -, 1263, 24813; cds-WP_002915991.1, NZ_KK737550.1, 298055, 298981, +, 927, 0; cds-WP_032428400.1, NZ_KK737550.1, 298947, 299660, -, 714, 348; cds-WP_002915993.1, NZ_KK737550.1, 299800, 300126, +, 327, 0; cds-WP_002915995.1, NZ_KK737550.1, 300579, 301469, +, 891, 58; cds-WP_002915996.1, NZ_KK737550.1, 301462, 302364, +, 903, 1777; cds-WP_021440680.1, NZ_KK737550.1, 302377, 303504, +, 1128, 1123; cds-WP_002915998.1, NZ_KK737550.1, 303521, 304807, +, 1287, 11755; cds-WP_032415517.1, NZ_KK737550.1, 304867, 306210, -, 1344, 0; cds-WP_026005853.1, NZ_KK737550.1, 306332, 307765, -, 1434, 2; cds-WP_002916000.1, NZ_KK737550.1, 307785, 309026, -, 1242, 18; cds-WP_004143987.1, NZ_KK737550.1, 309039, 309173, -, 135, 0; cds-WP_032428401.1, NZ_KK737550.1, 309268, 310263, +, 996, 14; cds-WP_002916001.1, NZ_KK737550.1, 310260, 311681, -, 1422, 16; cds-WP_002916003.1, NZ_KK737550.1, 311977, 312738, -, 762, 4233; cds-WP_002916048.1, NZ_KK737550.1, 313224, 314399, +, 1176, 345; cds-WP_004143995.1, NZ_KK737550.1, 314535, 315230, -, 696, 0; cds-WP_002916052.1, NZ_KK737550.1, 315479, 316657, -, 1179, 5957; cds-WP_002916055.1, NZ_KK737550.1, 316776, 317645, -, 870, 2; cds-WP_002916056.1, NZ_KK737550.1, 317748, 318209, +, 462, 10; cds-WP_002916058.1, NZ_KK737550.1, 318333, 319562, +, 1230, 42815; cds-WP_002916061.1, NZ_KK737550.1, 319681, 320559, +, 879, 584; cds-WP_013815099.1-72, NZ_KK737550.1, 320898, 321866, -, 969, 7025; cds-AF52_RS02175, NZ_KK737550.1, 321899, 322303, +, 405, 4; cds-WP_013815099.1-73, NZ_KK737550.1, 322616, 323584, +, 969, 6911; cds-WP_021440689.1, NZ_KK737550.1, 323835, 324443, +, 609, 603; cds-WP_002916190.1, NZ_KK737550.1, 324922, 325470, +, 549, 0; cds-WP_021440690.1, NZ_KK737550.1, 325541, 326077, +, 537, 0; cds-WP_002916192.1, NZ_KK737550.1, 326106, 326831, +, 726, 0; cds-WP_032430351.1, NZ_KK737550.1, 326913, 329525, +, 2613, 0; cds-WP_004185520.1, NZ_KK737550.1, 329536, 330063, +, 528, 0; cds-WP_004229447.1, NZ_KK737550.1, 330091, 330576, +, 486, 0; cds-WP_032423847.1, NZ_KK737550.1, 330594, 331496, +, 903, 0; cds-WP_021440694.1, NZ_KK737550.1, 331493, 332905, +, 1413, 23; cds-WP_002916201.1, NZ_KK737550.1, 332948, 333220, -, 273, 0; cds-WP_004144031.1, NZ_KK737550.1, 333232, 333501, -, 270, 161; cds-WP_002916204.1, NZ_KK737550.1, 333668, 333940, +, 273, 585; cds-WP_004174502.1, NZ_KK737550.1, 333947, 334972, +, 1026, 793; cds-WP_004185523.1, NZ_KK737550.1, 334985, 336922, +, 1938, 2299; cds-WP_032430352.1, NZ_KK737550.1, 336919, 338367, +, 1449, 13; cds-WP_002916274.1, NZ_KK737550.1, 338632, 338931, -, 300, 2022; cds-WP_002916277.1, NZ_KK737550.1, 339116, 340669, -, 1554, 456448; cds-WP_016947386.1, NZ_KK737550.1, 341142, 341564, -, 423, 1823; cds-WP_023301507.1, NZ_KK737550.1, 341574, 342866, -, 1293, 449; cds-WP_002916281.1, NZ_KK737550.1, 342918, 343247, -, 330, 0; cds-WP_002916282.1, NZ_KK737550.1, 343466, 343759, +, 294, 155; cds-WP_002916283.1, NZ_KK737550.1, 343790, 344362, +, 573, 2994; cds-WP_014599254.1, NZ_KK737550.1, 344441, 344644, +, 204, 0; cds-WP_004174493.1, NZ_KK737550.1, 344813, 345712, -, 900, 6439; cds-WP_004151785.1, NZ_KK737550.1, 345851, 346159, +, 309, 6; cds-WP_023285538.1, NZ_KK737550.1, 346168, 346725, +, 558, 2; cds-WP_032428403.1, NZ_KK737550.1, 346746, 347765, +, 1020, 0; cds-WP_004185540.1, NZ_KK737550.1, 347798, 348601, +, 804, 0; cds-WP_015958948.1, NZ_KK737550.1, 348965, 349894, -, 930, 3461; cds-AF52_RS02025, NZ_KK737550.1, 350024, 351103, +, 1080, 7; cds-WP_013815099.1-74, NZ_KK737550.1, 351161, 352129, -, 969, 5948; cds-AF52_RS26135, NZ_KK737550.1, 352161, 352478, +, 318, 0; cds-WP_004157874.1, NZ_KK737550.1, 352800, 353513, -, 714, 16674; cds-WP_002916298.1, NZ_KK737550.1, 353830, 354384, +, 555, 370; cds-WP_002916299.1, NZ_KK737550.1, 354620, 356137, -, 1518, 98981; cds-WP_095858446.1, NZ_KK737550.1;NZ_KK737550.1, 356147;357171, 357169;357245, -;-, 1098, 134972; cds-WP_004149758.1, NZ_KK737550.1, 357331, 359064, -, 1734, 33923; cds-WP_004185548.1, NZ_KK737550.1, 359070, 359783, -, 714, 8279; cds-WP_004144729.1, NZ_KK737550.1, 359806, 360702, -, 897, 5659; cds-WP_004144730.1, NZ_KK737550.1, 360803, 361324, +, 522, 7368; cds-WP_002916310.1, NZ_KK737550.1, 361331, 361741, -, 411, 4802; cds-
WP_002916312.1, NZ_KK737550.1, 361722, 361988, -, 267, 3366; cds-WP_004185555.1, NZ_KK737550.1, 362234, 363217, +, 984, 9759; cds-WP_002916317.1, NZ_KK737550.1, 363368, 364027, -, 660, 756; cds-WP_004144734.1, NZ_KK737550.1, 364191, 364502, -, 312, 0; cds-WP_002916321.1, NZ_KK737550.1, 364552, 365280, +, 729, 199; cds-WP_002916322.1, NZ_KK737550.1, 365399, 366832, +, 1434, 384; cds-WP_004185561.1, NZ_KK737550.1, 366957, 367316, +, 360, 0; cds-WP_009308616.1, NZ_KK737550.1, 367366, 369375, +, 2010, 1; cds-WP_009308617.1, NZ_KK737550.1, 369377, 369988, +, 612, 5; cds-WP_009308618.1, NZ_KK737550.1, 369978, 370481, +, 504, 1; cds-WP_004174442.1, NZ_KK737550.1, 370602, 371345, -, 744, 326; cds-WP_004900541.1, NZ_KK737550.1, 371408, 374281, -, 2874, 1279756; cds-WP_002916479.1, NZ_KK737550.1, 374488, 374877, -, 390, 455166; cds-WP_002916480.1, NZ_KK737550.1, 374902, 375996, -, 1095, 485020; cds-WP_002916481.1, NZ_KK737550.1, 376419, 377621, -, 1203, 4360; cds-WP_021440702.1, NZ_KK737550.1, 377632, 378810, -, 1179, 15; cds-WP_002916483.1, NZ_KK737550.1, 378807, 380123, -, 1317, 20014; cds-WP_002916486.1, NZ_KK737550.1, 380147, 380725, -, 579, 157; cds-WP_002916488.1, NZ_KK737550.1, 380892, 381221, +, 330, 7542; cds-WP_004144746.1, NZ_KK737550.1, 381545, 382141, +, 597, 13511; cds-WP_002916493.1, NZ_KK737550.1, 382225, 383457, -, 1233, 23718; cds-WP_002916495.1, NZ_KK737550.1, 383727, 384386, -, 660, 563; cds-WP_002916497.1, NZ_KK737550.1, 384554, 385447, +, 894, 193; cds-WP_002916499.1, NZ_KK737550.1, 385501, 386268, -, 768, 44696; cds-WP_004144747.1, NZ_KK737550.1, 386358, 386993, -, 636, 54; cds-WP_002916504.1, NZ_KK737550.1, 387145, 388002, -, 858, 6997; cds-WP_004144748.1, NZ_KK737550.1, 388193, 389272, -, 1080, 1627091; cds-WP_002916508.1, NZ_KK737550.1, 389370, 390533, -, 1164, 245189; cds-WP_002916512.1, NZ_KK737550.1, 390573, 391601, -, 1029, 34366; cds-WP_004174438.1, NZ_KK737550.1, 391987, 393978, -, 1992,494518; cds-WP_004151780.1, NZ_KK737550.1, 394257, 395015, +, 759, 1776; cds-WP_032428408.1, NZ_KK737550.1, 395393, 396661, +, 1269, 0; cds-WP_021440705.1, NZ_KK737550.1, 396798, 398438, -, 1641, 0; cds-WP_032430353.1, NZ_KK737550.1, 398452, 399921, -, 1470, 0; cds-WP_004211378.1, NZ_KK737550.1, 399937, 400701, -, 765, 0; cds-WP_032430354.1, NZ_KK737550.1, 400711, 401505, -, 795, 0; cds-WP_032428409.1, NZ_KK737550.1, 401489, 402259, -, 771, 0; cds-WP_032430355.1, NZ_KK737550.1, 402269, 402799, -, 531, 8; cds-WP_004149797.1, NZ_KK737550.1, 402810, 404066, -, 1257, 0; cds-WP_004174432.1, NZ_KK737550.1, 404093, 405289, -, 1197, 6; cds-WP_004149799.1, NZ_KK737550.1, 405286, 406212, -, 927, 12562; cds-WP_021440707.1, NZ_KK737550.1, 406301, 407089, -, 789, 21015; cds-WP_021440708.1, NZ_KK737550.1, 407103, 407852, -, 750, 26308; cds-WP_004174429.1, NZ_KK737550.1, 407852, 408874, -, 1023, 46956; cds-WP_004144765.1, NZ_KK737550.1, 408885, 409193, -, 309, 616; cds-WP_004151769.1, NZ_KK737550.1, 409190, 409513, -, 324, 528; cds-WP_032428411.1, NZ_KK737550.1, 409941, 411161, +, 1221, 9; cds-WP_002916572.1, NZ_KK737550.1, 411289, 412209, -, 921, 48275; cds-WP_004149805.1, NZ_KK737550.1, 412443, 414419, -, 1977, 7027; cds-WP_002916578.1, NZ_KK737550.1, 414428, 414559, -, 132, 42; cds-WP_157828762.1, NZ_KK737550.1, 414740, 415063, +, 324, 5408; cds-WP_004149807.1, NZ_KK737550.1, 415261, 416415, +, 1155, 21538; cds-WP_002916587.1, NZ_KK737550.1, 416803, 418197, +, 1395, 641; cds-WP_002916588.1, NZ_KK737550.1, 418275, 418775, +, 501, 76; cds-WP_002916589.1, NZ_KK737550.1, 418870, 419577, +, 708, 10; cds-WP_002916597.1, NZ_KK737550.1, 419669, 420400, +, 732, 7886; cds-WP _002916600.1, NZ_KK737550.1, 420421, 421371, +, 951, 49391; cds-WP_002916603.1, NZ_KK737550.1, 421546, 422109, +, 564, 7245; cds-WP_002916605.1, NZ_KK737550.1, 422109, 422525, +, 417, 57; cds-WP_002916607.1, NZ_KK737550.1, 422578, 423231, -, 654, 516; cds-WP_004149811.1, NZ_KK737550.1, 423580, 424560, -, 981, 0; cds-WP_004144780.1, NZ_KK737550.1, 424577, 425278, +, 702, 77; cds-WP_002916613.1, NZ_KK737550.1, 425299, 425865, +, 567, 4439; cds-WP _002916614.1, NZ_KK737550.1, 425862, 426152, +, 291, 19011; cds-WP_002916615.1, NZ_KK737550.1, 426165, 426758, +, 594, 23882; cds-WP_004149812.1, NZ_KK737550.1, 426751, 427887, +, 1137, 70; cds-WP_023285550.1, NZ_KK737550.1, 428193, 429179, +, 987, 12; cds-WP_025861936.1, NZ_KK737550.1, 429226, 429729, +, 504, 2; cds-WP_004149816.1, NZ_KK737550.1, 429729, 431030, +, 1302, 10349; cds-WP_019704316.1, NZ_KK737550.1, 431078, 431797, -, 720, 60049; cds-WP_002916620.1, NZ_KK737550.1, 431848, 432174, -, 327, 2853; cds-WP_002916627.1, NZ_KK737550.1, 432174, 432893, -, 720, 2359; cds-AF52_RS01615, NZ_KK737550.1, 433030, 434087, +, 1058, 1132; cds-WP_004105744.1, NZ_KK737550.1, 434116, 434391, +, 276, 11034; cds-WP_004900580.1, NZ_KK737550.1, 434441, 435523, +, 1083,32301; cds-WP_004144787.1, NZ_KK737550.1, 435715, 436968, +, 1254, 6484; cds-WP_009484134.1, NZ_KK737550.1, 437012, 439150, -, 2139, 13; cds-WP_002916694.1, NZ_KK737550.1, 439570, 440283, +, 714, 13995; cds-AF52_RS01580, NZ_KK737550.1, 441124, 441340, -, 217, 2353; cds-WP_004188553.1, NZ_KK737550.1, 441437, 442030, -, 594, 1903; cds-WP_021314147.1, NZ_KK737550.1, 442734, 443219, -, 486, 1924; cds-WP_004150912.1, NZ_KK737550.1, 443216, 443509, -, 294, 10; cds-AF52_RS27585, NZ_KK737550.1, 444170, 444344, +, 175, 348; cds-WP_032175380.1-2, NZ_KK737550.1, 445134, 445482, +, 349, 1650; cds-WP_004217775.1, NZ_KK737550.1, 445564, 446940, +, 1377, 13; cds-WP_004150915.1, NZ_KK737550.1, 446951, 448099, +, 1149, 4032; cds-WP_004188550.1, NZ_KK737550.1, 448100, 449011, -, 912, 0; cds-WP_021440720.1, NZ_KK737550.1, 449109, 449711, +, 603, 1; cds-WP_004174409.1, NZ_KK737550.1, 449798, 450142, +, 345, 3; cds-WP_002916738.1, NZ_KK737550.1, 450342, 450530, +, 189, 3250; cds-WP_004149825.1, NZ_KK737550.1, 450707, 451888, -, 1182, 747; cds-WP _002916740.1, NZ_KK737550.1, 452034, 453899, -, 1866, 21930; cds-WP_004217169.1, NZ_KK737550.1, 453976, 454098, -, 123, 0; cds-WP_004150918.1, NZ_KK737550.1, 454119, 454679, +, 561, 3; cds-WP_002916742.1, NZ_KK737550.1, 454727, 455593, +, 867, 404; cds-WP_002916743.1, NZ_KK737550.1, 455666, 455953, -, 288, 0; cds-WP _004144560.1, NZ_KK737550.1, 456032, 456919, -, 888, 1105; cds-WP_004105804.1, NZ_KK737550.1, 457041, 457535, -, 495, 1472; cds-WP_032412422.1, NZ_KK737550.1, 457642, 459165, -, 1524, 213; cds-WP _002916782.1, NZ_KK737550.1, 459179, 460597, -, 1419, 5729; cds-WP_002916785.1, NZ_KK737550.1, 460802, 461227, -, 426, 10923; cds-WP_004174395.1, NZ_KK737550.1, 461234, 461965, -, 732, 1464; cds-WP_004149836.1, NZ_KK737550.1, 462216, 463403, +, 1188, 108; cds-WP_002916791.1, NZ_KK737550.1, 463535, 464194, +, 660, 741; cds-WP_032430356.1, NZ_KK737550.1, 464244, 465143, -, 900, 4367; cds-WP_004144551.1, NZ_KK737550.1, 465340, 466503, +, 1164, 59; cds-WP _023286874.1, NZ_KK737550.1, 466649, 467476, +, 828, 0; cds-WP_004144550.1, NZ_KK737550.1, 467512, 468039, -, 528, 36; cds-WP_004174383.1, NZ_KK737550.1, 468096, 470279, -, 2184, 70985; cds-WP_004144547.1, NZ_KK737550.1, 470403, 471815, -, 1413, 32584; cds-WP_002916826.1, NZ_KK737550.1, 471899, 472636, -, 738, 64957; cds-WP_004181324.1, NZ_KK737550.1, 472828, 475086, -, 2259, 56355; cds-WP_002916831.1, NZ_KK737550.1, 475208, 476077, -, 870, 50; cds-WP _002916833.1, NZ_KK737550.1, 476155, 476550, -, 396, 4700; cds-WP_002916836.1, NZ_KK737550.1, 476701, 477360, +, 660, 38; cds-WP_021440728.1, NZ_KK737550.1, 477357, 478706, +, 1350, 0; cds-WP_002916840.1, NZ_KK737550.1, 478813, 479394, +, 582, 953; cds-WP_002916842.1, NZ_KK737550.1, 479475, 479789, +, 315, 34802; cds-WP_002916844.1, NZ_KK737550.1, 479862, 481757, -, 1896, 75575; cds-WP_002916845.1, NZ_KK737550.1, 481788, 482363, -, 576, 73233; cds-WP_002916846.1, NZ_KK737550.1, 482363, 483190, -, 828, 27868; cds-WP_002916847.1, NZ_KK737550.1, 483215, 483637, -, 423, 21885; cds-WP_002916848.1, NZ_KK737550.1, 483634, 484266, -, 633, 9449; cds-WP_004150921.1, NZ_KK737550.1, 484463, 485932, +, 1470, 123649; cds-WP_002916849.1, NZ_KK737550.1, 486100, 486771, +, 672, 24387; cds-WP_004900674.1, NZ_KK737550.1, 486777, 487937, +, 1161, 76494; cds-WP_004174357.1, NZ_KK737550.1, 487982, 488773, -, 792, 206; cds-WP_032430358.1, NZ_KK737550.1, 488960, 489730, +, 771, 35; cds-WP_004185664.1, NZ_KK737550.1, 489792, 490445, -, 654, 250; cds-WP_002916856.1, NZ_KK737550.1, 490823, 491095, +, 273, 7971; cds-WP_002916857.1, NZ_KK737550.1, 491131, 491328, -, 198, 11036; cds-AF52_RS28140, NZ_KK737550.1, 491320, 491470, +, 151, 0; cds-WP_002916858.1, NZ_KK737550.1, 491548, 492981, -, 1434, 11597; cds-WP 002916860.1, NZ_KK737550.1, 493040, 495877, -, 2838, 15222; cds-WP_009308672.1, NZ_KK737550.1, 495898, 497196, -, 1299, 65; cds-WP_002916862.1, NZ_KK737550.1, 497411, 498031, +, 621, 33678; cds-WP_002916864.1, NZ_KK737550.1, 498093, 499334, +, 1242, 23721; cds-WP_004144532.1, NZ_KK737550.1, 499346, 500167, -, 822, 8568; cds-WP_004150923.1, NZ_KK737550.1, 500366, 500749, -, 384, 3729; cds-WP_004144530.1, NZ_KK737550.1, 500857, 501474, +, 618, 3; cds-WP_021440736.1, NZ_KK737550.1, 501606, 501725, +, 120, 0; cds-AF52_RS27590, NZ_KK737550.1, 501736, 501798, -, 63, 0; cds-WP_013815099.1-75, NZ_KK737550.1, 501856, 502824, -, 969, 8190; cds-WP_032430359.1, NZ_KK737550.1, 502826, 502948, -, 123, 0; cds-WP_004174349.1, NZ_KK737550.1, 502938, 503762, +, 825, 2; cds-WP_002916871.1, NZ_KK737550.1, 503772, 504074, +, 303, 0; cds-WP_021440738.1, NZ_KK737550.1, 504084, 504404, +, 321, 0; cds-WP_015958999.1, NZ_KK737550.1, 504397, 506100, +, 1704, 1; cds-WP_004215466.1, NZ_KK737550.1, 506110, 506586, +, 477, 0; cds-WP_016532672.1, NZ_KK737550.1, 506588, 507262, +, 675, 1; cds-WP_002916877.1, NZ_KK737550.1, 507271, 507888, +, 618, 3362; cds-WP_025367880.1, NZ_KK737550.1, 508042, 509649, +, 1608, 3667; cds-WP_004900709.1, NZ_KK737550.1, 509694, 510707, -, 1014, 14; cds-WP _001144069.1, NZ_KK737550.1, 510945, 511160, +, 216, 42897; cds-WP_004149864.1, NZ_KK737550.1, 511272, 513017, +, 1746, 145901; cds-WP_004174339.1, NZ_KK737550.1, 513236, 515077, +, 1842, 329702; cds-WP_002917636.1, NZ_KK737550.1, 515177, 515683, -, 507, 0; cds-WP_004174338.1, NZ_KK737550.1, 516020, 516238, -, 219, 162; cds-WP_032430361.1, NZ_KK737550.1, 516309, 517406, -, 1098, 0; cds-WP_004185683.1, NZ_KK737550.1, 517403, 517888, -, 486, 0; cds-AF52_RS01195, NZ_KK737550.1, 517885, 519153, -, 1269, 2; cds-WP_020806188.1-6, NZ_KK737550.1, 519222, 520202, +, 981, 7952; cds-WP_050484295.1, NZ_KK737550.1, 520559, 522556, -, 1998, 468202; cds-WP_032430362.1, NZ_KK737550.1, 522572, 523600, -, 1029, 24116; cds-WP_032414744.1, NZ_KK737550.1, 523929, 524162, -, 234, 2424; cds-WP_032414845.1, NZ_KK737550.1, 524174, 524362, -, 189, 16; cds-WP_032430364.1, NZ_KK737550.1, 524546, 526954, -, 2409, 2; cds-AF52_RS27605, NZ_KK737550.1, 527242, 527414, +, 173, 566; cds-AF52_RS0124695, NZ_KK737550.1, 528005, 528295, +, 291, 3535; cds-WP_021441030.1, NZ_KK737550.1, 528625, 530883, +, 2259, 17; cds-WP _004177844.1, NZ_KK737550.1, 531147, 531515, -, 369, 3; cds-WP_004177843.1, NZ_KK737550.1, 531549, 533309, -, 1761, 511; cds-WP_004146634.1, NZ_KK737550.1, 533683, 533913, -, 231, 0; cds-WP_013815099.1-76, NZ_KK737550.1, 533996, 534964, -, 969, 3602; cds-WP_023285711.1, NZ_KK737550.1, 535001, 537424, +, 2424, 63511; cds-WP_013815099.1-77, NZ_KK737550.1, 537590, 538558, -, 969, 6373; cds-WP_004198387.1, NZ_KK737550.1, 538641, 539402, -, 762, 2; cds-WP_002885046.1, NZ_KK737550.1, 539413, 540426, -, 1014, 400; cds-WP_004151742.1, NZ_KK737550.1, 540423, 541181, -, 759, 0; cds-WP_032430384.1, NZ_KK737550.1, 541357, 542385, +, 1029, 0; cds-WP_013815099.1-78, NZ_KK737550.1, 542403, 543371, +, 969, 4083; cds-WP_004174268.1, NZ_KK737550.1, 543545, 543796, -, 252, 39; cds-WP_032430367.1, NZ_KK737550.1, 543799, 544413, -, 615, 1628; cds-WP_004174272.1, NZ_KK737550.1, 544514, 544750, +, 237, 7; cds-AF52_RS26555, NZ_KK737550.1, 544785, 545111, +, 327, 127; cds-WP_013815099.1-79, NZ_KK737550.1, 545145, 546113, +, 969, 4539; cds-AF52_RS0124710, NZ_KK737550.1, 546172, 546360, +, 189, 0; cds-WP_004174277.1, NZ_KK737550.1, 546368, 546568, +, 201, 0; cds-AF52_RS01075, NZ_KK737550.1, 546532, 546735, +, 204, 0; cds-WP_002885077.1, NZ_KK737551.1, 251, 709, -, 459, 7; cds-WP_004151738.1, NZ_KK737551.1, 794, 1123, +, 330, 4; cds-WP_023283136.1, NZ_KK737551.1, 1108, 2712, -, 1605, 2678; cds-WP_004177826.1, NZ_KK737551.1, 3219, 3500, +, 282, 0; cds-WP_004186517.1, NZ_KK737551.1, 3547,
4404, -, 858, 0; cds-WP _004177833.1, NZ_KK737551.1, 4401, 5234, -, 834, 5; cds-WP _004177835.1, NZ_KK737551.1, 5294, 6433, -, 1140, 0; cds-WP _013815099.1-80, NZ_KK737551.1, 6546, 7514, -, 969, 4148; cds-AF52_RS01030, NZ_KK737551.1, 7646, 8203, -, 558, 0; cds-AF52_RS01025, NZ_KK737551.1, 8492, 9028, -, 537, 35; cds-AF52_RS0124720, NZ_KK737551.1, 9021, 9488, -, 468, 0; cds-AF52_RS01020, NZ_KK737551.1, 10314, 10625, -, 312, 0; cds-AF52_RS26200, NZ_KK737551.1, 10743, 10920, +, 178, 407; cds-AF52_RS01015, NZ_KK737551.1, 11477, 12304, -, 828, 26; cds-AF52_RS01010, NZ_KK737551.1, 12683, 13012, +, 330, 0; cds-AF52_RS27655, NZ_KK737551.1, 13210, 13361, +, 152, 432; cds-AF52_RS01000, NZ_KK737551.1, 13462, 13718, +, 257, 623; cds-WP _002885500.1, NZ_KK737551.1, 14025, 15053, -, 1029, 8751; cds-WP _002885506.1, NZ_KK737551.1, 15095, 15661, +, 567, 64376; cds-WP_002885511.1, NZ_KK737551.1, 15735, 15866, +, 132, 376; cds-WP _002885518.1, NZ_KK737551.1, 16026, 16172, +, 147, 193; cds-WP_002885521.1, NZ_KK737551.1, 16316, 16633, +, 318, 11; cds-WP_002885523.1, NZ_KK737551.1, 16630, 17163, -, 534, 0; cds-WP_002885526.1, NZ_KK737551.1, 17277, 17636, -, 360, 4; cds-WP _002885530.1, NZ_KK737551.1, 17647, 18042, -, 396, 789; cds-WP_004177729.1, NZ_KK737551.1, 18053, 18787, -, 735, 7918; cds-WP_004177727.1, NZ _KK737551.1, 18780, 20570, -, 1791, 10998; cds-WP _004146714.1, NZ_KK737551.1, 20846, 21823, +, 978, 1; cds-WP _021441059.1, NZ_KK737551.1, 22006, 23508, +, 1503, 2; cds-WP _004146716.1, NZ_KK737551.1, 23546, 26875, -, 3330, 29627; cds-WP_002885536.1, NZ_KK737551.1, 26899, 27861, -, 963, 41581; cds-WP_002885537.1, NZ_KK737551.1, 28022, 29083, -, 1062, 966; cds-WP_002885538.1, NZ_KK737551.1, 29178, 29723, +, 546, 51; cds-WP _032430397.1, NZ_KK737551.1, 29767, 30498, -, 732, 2; cds-WP _004210690.1, NZ_KK737551.1, 31431, 32570, -, 1140, 11038; cds-WP_187079197.1, NZ_KK737551.1, 32584, 34095, +, 1512, 49; cds-WP _004220675.1, NZ_KK737551.1, 34100, 34549, +, 450, 2571; cds-WP _004152021.1, NZ_KK737551.1, 34566, 35921, +, 1356, 1073; cds-WP _004152020.1, NZ_KK737551.1, 35932, 37791, +, 1860, 1429; cds-WP _032430401.1, NZ_KK737551.1, 37784, 38734, +, 951, 68613; cds-WP_002885659.1, NZ_KK737551.1, 38827, 39135, +, 309, 121498; cds-WP _004146725.1, NZ_KK737551.1, 39211, 40491, +, 1281, 54204; cds-WP_004146726.1, NZ_KK737551.1, 40557, 41819, +, 1263, 154711; cds-WP_002885662.1, NZ_KK737551.1, 41822, 42826, +, 1005, 262957; cds-WP_002885663.1, NZ_KK737551.1, 42909, 43199, -, 291, 0; cds-WP _002885664.1, NZ_KK737551.1, 43422, 43619, +, 198, 0; cds-WP _002885665.1, NZ_KK737551.1, 43722, 45020, +, 1299, 44878; cds-WP_002885667.1, NZ_KK737551.1, 45171, 45596, +, 426, 5112; cds-WP _004192475.1, NZ_KK737551.1, 45633, 48065, +, 2433, 122281; cds-WP_002885669.1, NZ_KK737551.1, 48147, 48878, +, 732, 24469; cds-WP _021441063.1, NZ_KK737551.1, 49004, 50641, +, 1638, 8467; cds-WP _002885673.1, NZ_KK737551.1, 50632, 50907, -, 276, 10304; cds-WP_002885674.1, NZ_KK737551.1, 51045, 51353, -, 309, 9040; cds-WP _032428483.1, NZ_KK737551.1, 51563, 52279, +, 717, 4; cds-WP _004177697.1, NZ_KK737551.1, 52276, 53031, -, 756, 29; cds-WP _032430402.1, NZ_KK737551.1, 53139, 54203, -, 1065, 0; cds-WP_016946098.1, NZ_KK737551.1, 54566, 55966, +, 1401, 0; cds-WP _002885682.1, NZ_KK737551.1, 55979, 56284, +, 306, 0; cds-WP _002885683.1, NZ_KK737551.1, 56294, 56761, +, 468, 0; cds-WP _002885684.1, NZ_KK737551.1, 56775, 57425, +, 651, 6; cds-WP_004210671.1, NZ_KK737551.1, 57435, 58289, +, 855, 0; cds-WP_004178347.1, NZ_KK737551.1, 58289, 58975, +, 687, 1; cds-WP_009309325.1, NZ_KK737551.1, 59038, 60231, -, 1194, 0; cds-WP _002885689.1, NZ_KK737551.1, 60381, 60656, -, 276, 0; cds-WP _002885691.1, NZ_KK737551.1, 60997, 61392, +, 396, 265942; cds-WP _002885722.1, NZ_KK737551.1, 61398, 61712, +, 315, 341717; cds-WP _000135199.1, NZ_KK737551.1, 61717, 61944, +, 228, 358153; cds-WP_002886682.1, NZ_KK737551.1, 61986, 62435, +, 450, 277067; cds-WP_004177687.1, NZ_KK737551.1, 62544, 63218, -, 675, 2329; cds-WP _002886687.1, NZ_KK737551.1, 63437, 64057, +, 621, 843; cds-WP_002886688.1, NZ_KK737551.1, 64362, 65765, +, 1404, 190240; cds-WP_004210666.1, NZ_KK737551.1, 65870, 66532, -, 663, 2; cds-WP_002886691.1, NZ_KK737551.1, 66615, 67586, -, 972, 0; cds-WP_032430405.1, NZ_KK737551.1, 67662, 68486, -, 825, 0; cds-WP_021441067.1, NZ_KK737551.1, 68564, 69412, -, 849, 12; cds-WP _004152015.1, NZ_KK737551.1, 69502, 69873, +, 372, 6; cds-WP_021441068.1, NZ_KK737551.1, 69928, 71871, -, 1944, 11637; cds-WP _032430406.1, NZ_KK737551.1, 72063, 72806, +, 744, 5; cds-WP_002886706.1, NZ_KK737551.1, 72796, 73353, -, 558, 2212; cds-WP_002886707.1, NZ_KK737551.1, 73677, 73883, +, 207, 7; cds-WP_004178353.1, NZ_KK737551.1, 73945, 74817, -, 873, 11019; cds-WP_004222103.1, NZ_KK737551.1, 74848, 76857, -, 2010, 6; cds-WP_016946104.1, NZ_KK737551.1, 76876, 78108, -, 1233, 0; cds-WP _002886718.1, NZ_KK737551.1, 78310, 79647, -, 1338, 628; cds-WP _002886721.1, NZ_KK737551.1, 79828, 80466, -, 639, 112; cds-WP _002886722.1, NZ_KK737551.1, 80675, 82408, +, 1734, 3680; cds-WP_032430407.1, NZ_KK737551.1, 82405, 86181, +, 3777, 23289; cds-WP _002886724.1, NZ_KK737551.1, 86184, 86528, +, 345, 90; cds-WP_021441069.1, NZ_KK737551.1, 86566, 87039, -, 474, 60; cds-WP_171789277.1, NZ_KK737551.1, 87011, 87733, -, 723, 53; cds-WP_02144107.1, NZ_KK737551.1, 87889, 89076, -, 1188, 4947; cds-WP _002886766.1, NZ_KK737551.1, 89151, 89681, -, 531, 43916; cds-WP_002886769.1, NZ_KK737551.1, 90085, 91041, +, 957, 2647; cds-AF52_RS00620, NZ_KK737551.1, 91165, 92456, +, 1292, 0; cds-WP_032430409.1, NZ_KK737551.1, 92467, 93492, +, 1026,0; cds-WP_021312675.1, NZ_KK737551.1, 93479, 94477, +, 999, 270; cds-WP_002886827.1, NZ_KK737551.1, 94520, 95518, -, 999, 109287; cds-WP_002886886.1, NZ_KK737551.1, 95694, 97067, +, 1374, 1293; cds-WP _004177666.1, NZ_KK737551.1, 97136, 97663, +, 528, 17; cds-WP_032430410.1, NZ_KK737551.1, 97707, 98915, -, 1209, 3723; cds-WP _004177664.1, NZ_KK737551.1, 99407, 99958, -, 552, 14403; cds-WP _004152265.1, NZ_KK737551.1, 100054, 101406, +, 1353, 4104; cds-WP_021441074.1, NZ_KK737551.1, 101443, 101829, +, 387, 46482; cds-WP _002886894.1, NZ _KK737551.1, 102119, 102457, +, 339, 58; cds-WP_002886895.1, NZ_KK737551.1, 102468, 102830, +, 363, 3; cds-WP_002886896.1, NZ _KK737551.1, 102833, 103132, +, 300, 148; cds-WP_002886897.1, NZ_KK737551.1, 103154, 103930, +, 777, 30155; cds-WP_004152267.1, NZ_KK737551.1, 103944, 104588, +, 645, 369; cds-WP _004152268.1, NZ_KK737551.1, 104694, 105827, +, 1134, 21293; cds-WP_021441075.1, NZ_KK737551.1, 105811, 106929, +, 1119, 629; cds-WP_002886899.1, NZ_KK737551.1, 106926, 107666, +, 741, 445; cds-WP_002886900.1, NZ_KK737551.1, 107733, 108887, +, 1155, 16807; cds-WP _004152270.1, NZ_KK737551.1, 108910, 110820, +, 1911, 131; cds-WP _032430412.1, NZ_KK737551.1, 110898, 111140, +, 243, 513; cds-WP_002886902.1, NZ_KK737551.1, 111130, 111414, +, 285, 646; cds-WP _004222151.1, NZ_KK737551.1, 111418, 111882, -, 465, 33846; cds-WP_004186701.1, NZ_KK737551.1, 112190, 114328, -, 2139, 778; cds-WP _021441079.1, NZ _KK737551.1, 114685, 115428, -, 744, 24040; cds-WP _032410138.1, NZ _KK737551.1, 115431, 115604, -, 174, 0; cds-WP _004178377.1, NZ_KK737551.1, 115689, 115997, +, 309, 1; cds-WP_021441081.1, NZ _KK737551.1, 116088, 117395, +, 1308, 65; cds-WP_004146783.1, NZ_KK737551.1, 117422, 118774, +, 1353, 40159; cds-WP _004178379.1, NZ_KK737551.1, 118830, 120485, -, 1656, 347602; cds-WP _021441082.1, NZ_KK737551.1, 120537, 121955, -, 1419, 514974; cds-WP_004177658.1, NZ_KK737551.1, 122086, 123033, -, 948, 24; cds-WP _009309303.1, NZ_KK737551.1, 123413, 126121, +, 2709, 11; cds-WP_002886926.1, NZ_KK737551.1, 126195, 126581, -, 387, 11840; cds-WP _002886927.1, NZ_KK737551.1, 126734, 127195, -, 462, 30992; cds-WP _002886928.1, NZ_KK737551.1, 127209, 128144, -, 936, 33057; cds-WP_002886930.1, NZ_KK737551.1, 128181, 128285, -, 105, 0; cds-WP_002886939.1, NZ_KK737551.1, 128480, 128932, +, 453, 0; cds-WP _002886940.1, NZ_KK737551.1, 129175, 130149, +, 975, 1; cds-WP _002886941.1, NZ_KK737551.1, 130161, 130991, +, 831, 0; cds-WP _021441084.1, NZ_KK737551.1, 131086, 132651, +, 1566, 0; cds-WP _004152274.1, NZ_KK737551.1, 132708, 134126, +, 1419, 34; cds-WP_002886945.1, NZ_KK737551.1, 134211, 134936, +, 726, 65335; cds-WP_002886946.1, NZ_KK737551.1, 134939, 135943, -, 1005, 33; cds-WP _002886947.1, NZ_KK737551.1, 136107, 136529, +, 423, 16138; cds-WP _004186726.1, NZ_KK737551.1, 136588, 137352, +, 765, 19575; cds-WP_004152275.1, NZ_KK737551.1, 137625, 138128, -, 504, 1968; cds-WP_032430414.1, NZ_KK737551.1, 138249, 141104, -, 2856, 69623; cds-WP_002886953.1, NZ_KK737551.1, 141104, 141547, -, 444,792; cds-WP_002886954.1, NZ_KK737551.1, 141667, 143178, -, 1512,75760; cds-WP_002886955.1, NZ_KK737551.1, 143568, 144665, +, 1098, 17; cds-WP_002886956.1, NZ_KK737551.1, 144665, 145747, +, 1083, 1880; cds-WP_002886957.1, NZ_KK737551.1, 145796, 147298, -, 1503, 4014; cds-WP_032430415.1, NZ_KK737551.1, 147397, 148218, -, 822, 168; cds-WP_004177655.1, NZ_KK737551.1, 148569, 150074, +, 1506, 53557; cds-WP _004186731.1, NZ_KK737551.1, 150093, 150902, +, 810, 45259; cds-WP _004152190.1, NZ_KK737551.1, 151887, 152744, -, 858, 18344; cds-WP_004146797.1, NZ_KK737551.1, 152896, 154809, -, 1914, 36641; cds-WP _004186736.1, NZ_KK737551.1, 155315, 157255, +, 1941, 125477; cds-WP _004186740.1, NZ_KK737551.1, 157302, 158315, +, 1014, 121336; cds-WP_021314296.1, NZ_KK737551.1, 158357, 159241, +, 885, 151443; cds-WP _002886970.1, NZ_KK737551.1, 159265, 160164, +, 900, 19098; cds-WP _021441086.1, NZ _KK737551.1, 160308, 161618, -, 1311, 8; cds-WP _021441087.1, NZ_KK737551.1, 161758, 162828, -, 1071, 0; cds-WP_032430419.1, NZ_KK737551.1, 162834, 163658, -, 825, 0; cds-WP_004177643.1, NZ_KK737551.1, 163669, 164556, -, 888, 0; cds-WP _012737244.1, NZ_KK737551.1, 164546, 165430, -, 885, 0; cds-WP _004178400.1, NZ_KK737551.1, 165561, 166580, -, 1020, 74; cds-WP_021441090.1, NZ_KK737551.1, 166692, 168161, -, 1470, 0; cds-WP_032428490.1, NZ_KK737551.1, 168158, 168832, -, 675, 1; cds-AF52_RS00275, NZ_KK737551.1, 169708, 170763, +, 1056, 249; cds-WP _013815099.1-81, NZ_KK737551.1, 170797, 171765, +, 969, 9785; cds-WP_002887608.1, NZ_KK737551.1, 171853, 172632, +, 780, 12738; cds-WP _032430421.1, NZ_KK737551.1, 172610, 173461, +, 852, 0; cds-WP_032428518.1, NZ_KK737551.1, 173474, 174535, +, 1062, 0; cds-WP_004146946.1, NZ_KK737551.1, 174553, 175563, +, 1011, 0; cds-WP _021441152.1, NZ_KK737551.1, 175697, 176629, +, 933, 442; cds-WP_002887618.1, NZ_KK737551.1, 176684,178975, -, 2292, 164239; cds-WP_002887620.1, NZ_KK737551.1, 179233, 179724, -, 492, 256; cds-WP_002887623.1, NZ_KK737551.1, 179790, 180527, -, 738, 3495; cds-WP_004178536.1, NZ_KK737551.1, 180530, 181069, -, 540, 0; cds-WP _002887631.1, NZ_KK737551.1, 181172, 181645, -, 474, 1; cds-WP_004146059.1, NZ_KK737551.1, 181636, 182412, -, 777, 677; cds-WP_032430423.1, NZ_KK737551.1, 182692, 183876, +, 1185, 47458; cds-WP_009485342.1, NZ_KK737551.1, 183893, 184774, +, 882, 203; cds-WP_002887645.1, NZ_KK737551.1, 185040, 185762, +, 723, 7; cds-WP _002887647.1, NZ_KK737551.1, 185723, 186397, +, 675, 104; cds-WP_004151383.1, NZ_KK737551.1, 186403, 186861, -, 459, 44; cds-AF52_RS0124785, NZ_KK737551.1, 187200, 187601, +, 402, 0; cds-WP _013815099.1-82, NZ_KK737551.1, 187633, 188601, +, 969, 5644; cds-AF52_RS00180, NZ_KK737551.1, 188659, 189609, +, 951, 14223; cds-WP_002887652.1, NZ_KK737551.1, 189659, 190447, -, 789, 140; cds-AF52_RS27680, NZ_KK737551.1, 190554, 190682, -, 129, 0; cds-WP_013815099.1-83, NZ_KK737551.1, 190741, 191709, -, 969, 5790; cds-AF52_RS00165, NZ_KK737551.1, 191743, 192666, -, 924, 37; cds-WP_004221147.1, NZ _KK737551.1, 192690, 192860, -, 171, 0; cds-WP_002887654.1, NZ_KK737551.1, 192931, 193170, +, 240, 21; cds-WP_023317511.1, NZ_KK737551.1, 193450, 194478, -, 1029, 54524; cds-WP _015959390.1, NZ_KK737551.1, 194581, 194994, +, 414, 34; cds-WP_021441160.1, NZ_KK737551.1, 194963, 195409, +, 447, 11352; cds-WP_004146044.1, NZ_KK737551.1, 195424, 196101, +, 678, 3437; cds-WP_002887711.1, NZ_KK737551.1, 196192, 197781, +, 1590, 27045; cds-WP _032430427.1, NZ _KK737551.1, 198001, 198621, +, 621, 2204; cds-WP_002887716.1, NZ _KK737551.1, 198751, 198912, +, 162, 149; cds-WP _021441162.1, NZ_KK737551.1, 199066, 200139, +, 1074, 31152; cds-WP _004177496.1, NZ_KK737551.1, 200136, 200930, +, 795, 6292; cds-WP_004146034.1, NZ_KK737551.1, 201061, 202338, +, 1278, 98209; cds-WP_004146033.1, NZ_KK737551.1, 202734, 203534, +, 801, 1474; cds-WP_004178548.1, NZ_KK737551.1, 203648, 204970, +, 1323, 790; cds-WP_004151380.1, NZ_KK737551.1,
205023, 206246, +, 1224, 22154; cds-WP _002887789.1, NZ_KK737551.1, 206349, 207068, +, 720, 109091; cds-WP_032430428.1, NZ_KK737551.1, 207148, 208164, -, 1017, 4360; cds-WP_002887794.1, NZ_KK737551.1, 208164, 208811, -, 648, 30; cds-WP _004177487.1, NZ_KK737551.1, 208916, 209887, +, 972, 30693; cds-WP _002887800.1, NZ_KK737551.1, 209897, 211279, +, 1383, 11; cds-WP_002887802.1, NZ_KK737551.1, 211301, 212533, +, 1233, 30991; cds-WP _002887805.1, NZ_KK737551.1, 212627, 214294, -, 1668, 338131; cds-WP_002887806.1, NZ_KK737551.1, 214523, 216460, +, 1938, 3933; cds-WP_002887809.1, NZ_KK737551.1, 216549, 216878, +, 330, 3; cds-WP _002887810.1, NZ_KK737551.1, 216865, 217380, -, 516, 675; cds-WP_002887811.1, NZ_KK737551.1, 217494, 218141, +, 648, 31052; cds-WP_002887812.1, NZ_KK737551.1, 218138, 219007, -, 870, 18661; cds-WP_002887814.1, NZ_KK737551.1, 219217, 219690, +, 474, 1737; cds-WP_002887820.1, NZ_KK737551.1, 219703, 220392, +, 690, 2071; cds-WP _021441167.1, NZ_KK737551.1, 220392, 221816, +, 1425, 19621; cds-WP_004214415.1, NZ_KK737551.1, 221877, 223235, +, 1359, 41; cds-WP_002887843.1, NZ_KK737551.1, 223298, 224014, -, 717, 295117; cds-WP_004146984.1, NZ_KK737551.1, 224110, 224250, +, 141, 33720; cds-WP_002887846.1, NZ_KK737551.1, 224647, 225333, +, 687, 136;

TABLE 10

Counts table of the large (1 million) K. pneumoniae library when analyzed in bulk. Table organized as Geneid, Chr, Start, End, Strand, Length, K.pneumoniae_Large; Geneid, Chr, Start, End, Strand, Length, K.pneumoniae_Large; etc
cds-AF52_RS22895, NZ_KK737546.1, 1, 1202, +, 1202, 1555; cds-AF52_RS22890, NZ_KK737546.1, 1257, 1415, -, 159, 4951; cds-AF52_RS26590, NZ_KK737546.1, 6165, 6338, -, 174, 7449; cds-AF52_RS26600, NZ_KK737546.1, 8440, 8543, -, 104, 4687; cds-AF52_RS0122900, NZ_KK737546.1, 8577, 10166, -, 1590, 2727; cds-WP _014228879.1, NZ _KK737546.1, 10308, 10652, -, 345, 13349; cds-WP_032429382.1, NZ_KK737546.1, 10695, 10889, -, 195, 0; cds-AF52_RS0122905, NZ_KK737546.1, 11334, 11537, +, 204, 2052; cds-AF52_RS26610, NZ_KK737546.1, 11581, 11765, +, 185, 7359; cds-AF52_RS22870, NZ_KK737546.1, 12344, 12603, +, 260, 11894; cds-WP_040234937.1, NZ_KK737546.1, 13181, 13495, +, 315, 4896; cds-WP _032429384.1, NZ_KK737546.1, 13777, 14166, -, 390, 80873; cds-WP_072001558.1, NZ_KK737546.1, 14276, 14503, +, 228, 112; cds-WP _032429385.1, NZ_KK737546.1, 14506, 15060, +, 555, 31; cds-WP_072001559.1, NZ_KK737546.1, 15105, 16124, +, 1020, 27396; cds-WP _032429698.1, NZ_KK737546.1, 16117, 16581, +, 465, 8498; cds-WP_032429386.1, NZ_KK737546.1, 16595, 17035, +, 441, 39166; cds-WP_013815099.1, NZ_KK737546.1, 17339, 18307, -, 969, 102841; cds-WP _004147997.1, NZ_KK737546.1, 18343, 18546, -, 204, 3160; cds-WP_139042930.1, NZ_KK737546.1, 19315, 19434, -, 120, 1002; cds-WP_016160648.1, NZ_KK737546.1, 20352, 20567, +, 216, 0; cds-WP_032429387.1, NZ_KK737546.1, 20567, 21064, +, 498, 0; cds-WP_016160650.1, NZ_KK737546.1, 21061, 21411, +, 351, 1257; cds-WP _050484272.1, NZ_KK737546.1, 22361, 22786, +, 426, 27052; cds-WP _013815099.1-2, NZ_KK737546.1, 22812, 23780, -, 969, 85375; cds-WP _023289182.1, NZ_KK737546.1, 23903, 24127, -, 225, 13958; cds-WP_162924227.1, NZ_KK737546.1, 24306, 24470, +, 165, 2597; cds-AF52_RS24930, NZ_KK737546.1, 24454, 24816, +, 363, 8491; cds-WP _023289183.1, NZ_KK737546.1, 24768, 25091, +, 324, 72; cds-WP _023289184.1, NZ_KK737546.1, 25088, 25519, +, 432, 0; cds-WP_012542168.1, NZ_KK737546.1, 25767, 26201, +, 435, 0; cds-WP _021462603.1, NZ_KK737546.1, 26201, 27922, +, 1722, 1474; cds-WP _017898992.1, NZ_KK737546.1, 27916, 28095, +, 180, 0; cds-WP_012542166.1, NZ_KK737546.1, 28095, 29354, +, 1260, 34; cds-WP_032429388.1, NZ_KK737546.1, 29391, 30311, +, 921, 0; cds-WP _032429389.1, NZ_KK737546.1, 30389, 31675, +, 1287, 1730; cds-WP _014907814.1, NZ_KK737546.1, 31734, 31994, +, 261, 0; cds-WP _020317538.1, NZ_KK737546.1, 31975, 32292, +, 318, 0; cds-WP _014228910.1, NZ_KK737546.1, 32289, 32627, +, 339, 0; cds-WP _032429390.1, NZ_KK737546.1, 32608, 32997, +, 390, 0; cds-WP _017898997.1, NZ_KK737546.1, 32994, 33395, +, 402, 0; cds-WP_014228913.1, NZ_KK737546.1, 33427, 33888, +, 462, 2085; cds-WP_014228914.1, NZ_KK737546.1, 33946, 34311, +, 366, 0; cds-WP _032429392.1, NZ_KK737546.1, 34544, 37879, +, 3336, 311; cds-WP _014228916.1, NZ_KK737546.1, 37879, 38217, +, 339, 0; cds-WP _032429393.1, NZ_KK737546.1, 38214, 38969, +, 756, 27; cds-WP_023317958.1, NZ_KK737546.1, 38971, 39681, +, 711, 0; cds-WP _032429394.1, NZ_KK737546.1, 39714, 40061, +, 348, 405; cds-WP_023159867.1, NZ_KK737546.1, 40113, 40706, +, 594, 0; cds-AF52_RS0122945, NZ_KK737546.1, 40769, 53314, +, 12546, 37418; cds-WP _032429396.1, NZ_KK737546.1, 53376, 54872, +, 1497, 16398; cds-WP _004892953.1, NZ_KK737546.1, 55027, 55179, +, 153, 4085; cds-WP _023279886.1, NZ_KK737546.1, 55452, 56165, -, 714, 209; cds-WP _004190914.1, NZ_KK737546.1, 56162, 56554, -, 393, 0; cds-WP _004150797.1, NZ_KK737546.1, 56547, 56870, -, 324, 41; cds-AF52_RS0122950, NZ_KK737546.1, 56993, 57166, -, 174, 7048; cds-WP_004140530.1, NZ_KK737546.1, 57320, 57547, -, 228, 1026; cds-WP_021462619.1, NZ_KK737546.1, 57660, 58853, -, 1194, 60; cds-WP_004150795.1, NZ_KK737546.1, 59476, 59661, +, 186, 600; cds-WP_004148027.1, NZ_KK737546.1, 59752, 60246, +, 495, 403; cds-WP _004140514.1, NZ_KK737546.1, 60273, 60779, +, 507, 6640; cds-WP _013815099.1-3, NZ_KK737546.1, 60849, 61817, +, 969, 75419; cds-WP _032429398.1, NZ_KK737546.1, 61874, 62749, +, 876, 58; cds-WP_004140511.1, NZ_KK737546.1, 62805, 64211, +, 1407, 445; cds-WP _004183659.1, NZ_KK737546.1, 64208, 65218, +, 1011, 1247; cds-WP _004140506.1, NZ_KK737546.1, 65334, 65531, -, 198, 2840; cds-WP_004183660.1, NZ_KK737546.1, 66098, 66730, +, 633, 3615; cds-WP _004140501.1, NZ_KK737546.1, 66770, 66949, +, 180, 855; cds-WP_004150790.1, NZ_KK737546.1, 67347, 68033, +, 687, 93103; cds-WP_004150789.1, NZ_KK737546.1, 68146, 68310, -, 165, 37565; cds-WP _004140497.1, NZ_KK737546.1, 68344, 69852, -, 1509, 1479; cds-WP _021313530.1, NZ_KK737546.1, 69973, 70863, +, 891, 1942; cds-WP_004213090.1, NZ_KK737546.1, 70870, 72654, -, 1785, 63584; cds-WP _004140494.1, NZ_KK737546.1, 72728, 73936, -, 1209, 66557; cds-WP_004179363.1, NZ_KK737546.1, 74239, 75282, +, 1044, 3046; cds-WP _004148038.1, NZ_KK737546.1, 75944, 76858, +, 915, 123755; cds-WP_004150783.1, NZ_KK737546.1, 76948, 77586, +, 639, 208926; cds-WP_004140489.1, NZ_KK737546.1, 77717, 77980, +, 264, 192381; cds-WP_004140488.1, NZ_KK737546.1, 78039, 78164, -, 126, 279454; cds-WP _004213093.1, NZ_KK737546.1, 78282, 78356, -, 75, 12500; cds-WP _004150782.1, NZ_KK737546.1, 78356, 78457, -, 102, 25410; cds-WP _004176549.1, NZ_KK737546.1, 78515, 79528, -, 1014, 152388; cds-WP _004176548.1, NZ_KK737546.1, 79829, 80068, +, 240, 46471; cds-WP_014343000.1, NZ_KK737546.1, 80058, 80396, -, 339, 65296; cds-WP _020802835.1, NZ_KK737546.1, 80401, 80910, -, 510, 1570; cds-WP _021441569.1, NZ_KK737546.1, 81056, 81748, +, 693, 7422; cds-WP _020324105.1, NZ_KK737546.1, 81780, 82955, -, 1176, 2444; cds-WP_004140469.1, NZ_KK737546.1, 83063, 83857, +, 795, 44816; cds-WP_002901080.1, NZ_KK737546.1, 83841, 84287, -, 447, 36130; cds-WP _002901088.1, NZ_KK737546.1, 84404, 84904, -, 501, 3546; cds-WP _072271772.1, NZ_KK737546.1, 85151, 86287, +, 1137, 143446; cds-WP _002901096.1, NZ_KK737546.1, 86458, 86700, -, 243, 95784; cds-WP _002901192.1, NZ_KK737546.1, 86971, 87390, +, 420, 29751; cds-WP _004152360.1, NZ_KK737546.1, 87393, 88658, +, 1266, 56084; cds-WP_021441571.1, NZ_KK737546.1, 88665, 89570, -, 906, 37665; cds-WP _004176542.1, NZ_KK737546.1, 89737, 90486, +, 750, 800; cds-WP _021441573.1, NZ_KK737546.1, 90483, 91700, -, 1218, 2634; cds-WP _004179386.1, NZ_KK737546.1, 91876, 92757, +, 882, 2600; cds-WP _004220356.1, NZ_KK737546.1, 92828, 92947, -, 120, 3032; cds-WP _016946410.1, NZ_KK737546.1, 93015, 93326, +, 312, 811; cds-WP _032428825.1, NZ_KK737546.1, 93618, 94166, +, 549, 840; cds-WP_124053788.1, NZ_KK737546.1, 94180, 94551, +, 372, 1124; cds-WP_004140429.1, NZ_KK737546.1, 94548, 95831, -, 1284, 242853; cds-WP _002901234.1, NZ_KK737546.1, 95917, 97851, -, 1935, 499238; cds-WP _002901236.1, NZ_KK737546.1, 98268, 99014, +, 747, 387979; cds-WP _072143276.1, NZ_KK737546.1, 99145, 99960, +, 816, 30285; cds-WP_002901240.1, NZ_KK737546.1, 100017, 100901, -, 885, 410850; cds-WP_002901243.1, NZ_KK737546.1, 100976, 101971, -, 996, 12440905; cds-WP _002901246.1, NZ_KK737546.1, 102310, 102723, +, 414, 304542; cds-WP_002901249.1, NZ_KK737546.1, 102767, 103045, +, 279, 150857; cds-WP_002901254.1, NZ_KK737546.1, 103140, 103484, -, 345, 408912; cds-WP _002901255.1, NZ_KK737546.1, 103645, 104898, -, 1254, 63845; cds-WP _004140413.1, NZ_KK737546.1, 105070, 105711, -, 642, 361642; cds-WP _004176535.1, NZ_KK737546.1, 105721, 106740, -, 1020, 813894; cds-WP_004140411.1, NZ_KK737546.1, 106879, 108732, -, 1854, 325252; cds-WP_004176531.1, NZ_KK737546.1, 108898, 109449, +, 552, 24526; cds-WP_002901387.1, NZ_KK737546.1, 110185, 110931, -, 747, 295366; cds-WP_004176528.1, NZ_KK737546.1, 111157, 112200, +, 1044, 160209; cds-WP _032428827.1, NZ_KK737546.1, 112205, 114151, +, 1947, 280801; cds-WP _004140406.1, NZ _KK737546.1, 114331, 115311, +, 981, 73875; cds-WP _004176526.1, NZ_KK737546.1, 115355, 116698, -, 1344,88096; cds-WP _004183673.1, NZ _KK737546.1, 116881, 117291, -, 411, 71; cds-WP _002901398.1, NZ_KK737546.1, 117378, 118001, +, 624, 6220; cds-WP _032429402.1, NZ_KK737546.1, 118053, 119360, -, 1308, 12699; cds-WP _032428829.1, NZ_KK737546.1, 119454, 120086, -, 633, 1288; cds-WP _004892845.1, NZ_KK737546.1, 120086, 121621, -, 1536, 22; cds-WP_004224226.1, NZ_KK737546.1, 121594, 122772, -, 1179, 1; cds-WP _004176519.1, NZ_KK737546.1, 122775, 123335, -, 561, 2315; cds-WP _004151923.1, NZ_KK737546.1, 123332, 124042, -, 711, 286; cds-WP _021441584.1, NZ_KK737546.1, 124029, 124685, -, 657, 46325; cds-WP_002901486.1, NZ_KK737546.1, 124899, 125705, -, 807, 79436; cds-WP_002901489.1, NZ_KK737546.1, 126144, 127364, +, 1221, 100254; cds-WP _032429405.1, NZ_KK737546.1, 127361, 128395, +, 1035, 167730; cds-WP _004151922.1, NZ_KK737546.1, 128392, 129870, +, 1479, 66885; cds-WP_004892826.1, NZ_KK737546.1, 129867, 131192, +, 1326, 115895; cds-WP_021441588.1, NZ_KK737546.1, 131202, 132167, +, 966, 8587; cds-WP _004217130.1, NZ _KK737546.1, 132511, 132996, +, 486, 449436; cds-WP_004176514.1, NZ_KK737546.1, 133084, 133947, -, 864, 11332; cds-WP _032429406.1, NZ _KK737546.1, 134048, 134875, -, 828, 311552; cds-WP_002901544.1, NZ_KK737546.1, 135158, 135496, +, 339, 42067; cds-WP _013815099.1-4, NZ_KK737546.1, 135868, 136836, +, 969, 101925; cds-WP_050484274.1, NZ_KK737546.1, 136880, 137161, +, 282, 5100; cds-WP_014907779.1, NZ_KK737546.1, 137181, 137297, +, 117, 0; cds-WP_004148091.1, NZ_KK737546.1, 137309, 138667, +, 1359, 471; cds-WP_004183683.1, NZ_KK737546.1, 138721, 139068, +, 348, 0; cds-WP _002901552.1, NZ_KK737546.1, 139096, 139920, +, 825, 3449; cds-WP_032429409.1, NZ_KK737546.1, 140031, 141377, +, 1347, 42638; cds-WP _004179430.1, NZ_KK737546.1, 141390, 142148, +, 759, 45019; cds-WP _002901554.1, NZ _KK737546.1, 142206, 144464, -, 2259, 254605; cds-WP _004140343.1, NZ_KK737546.1, 144577, 144909, +, 333, 680; cds-WP_032429412.1, NZ_KK737546.1, 144969, 146360, -, 1392, 395944; cds-WP _002901611.1, NZ_KK737546.1, 146496, 147086, -, 591, 58496; cds-WP_004176511.1, NZ_KK737546.1, 147178, 147939, -, 762, 29800; cds-
WP_004140335.1, NZ_KK737546.1, 148089, 148757, -, 669, 24478; cds-WP_002901627.1, NZ_KK737546.1, 148946, 149482, +, 537, 29062; cds-WP_002901629.1, NZ_KK737546.1, 149512, 149910, -, 399, 85319; cds-AF52_RS0123015, NZ_KK737546.1, 150010, 152194, -, 2185, 7964; cds-WP_002901631.1, NZ_KK737546.1, 152466, 153005, -, 540, 184626; cds-WP_002901632.1, NZ_KK737546.1, 153060, 153803, -, 744, 145060; cds-WP_032429413.1, NZ_KK737546.1, 153829, 154242, -, 414, 66217; cds-WP_002901634.1, NZ_KK737546.1, 154512, 155150, +, 639, 12747; cds-WP_032429414.1, NZ_KK737546.1, 155235, 155912, -, 678, 24863; cds-WP_032429415.1, NZ_KK737546.1, 155961, 158246, -, 2286, 54916; cds-WP_021441592.1, NZ_KK737546.1, 158556, 159263, -, 708, 23999; cds-WP_004151911.1, NZ_KK737546.1, 159382, 159582, +, 201, 937; cds-WP_032429416.1, NZ_KK737546.1, 159691, 160638, -, 948, 384; cds-WP_021441594.1, NZ_KK737546.1, 160713, 161171, +, 459, 14421; cds-WP_004183698.1, NZ_KK737546.1, 161214, 162122, -, 909, 4716; cds-WP_004210755.1, NZ_KK737546.1, 162251, 163561, +, 1311, 347; cds-WP_002901724.1, NZ_KK737546.1, 163576, 164664, +, 1089, 2042; cds-WP_004179454.1, NZ_KK737546.1, 164667, 165926, +, 1260, 53268; cds-WP_021441596.1, NZ_KK737546.1, 166145, 166954, -, 810, 147921; cds-WP_032423750.1, NZ_KK737546.1, 166954, 168147, -, 1194, 118962; cds-WP_032429417.1, NZ_KK737546.1, 168157, 169515, -, 1359, 37404; cds-WP_021441599.1, NZ_KK737546.1, 169519, 171114, -, 1596, 36142; cds-WP_004176488.1, NZ_KK737546.1, 171114, 172676, -, 1563, 67818; cds-WP_002901735.1, NZ_KK737546.1, 172965, 173828, +, 864, 123250; cds-WP_002901738.1, NZ_KK737546.1, 173845, 174465, +, 621, 216148; cds-WP_021441600.1, NZ_KK737546.1, 174487, 175395, -, 909, 30914; cds-WP_004176482.1, NZ_KK737546.1, 175507, 176403, +, 897, 5554; cds-WP_002901746.1, NZ_KK737546.1, 176685, 177587, +, 903, 317805; cds-WP_002901749.1, NZ_KK737546.1, 177648, 178238, -, 591, 131448; cds-WP_032429418.1, NZ_KK737546.1, 178235, 178996, -, 762, 505792; cds-WP_004148112.1, NZ_KK737546.1, 178991, 179206, +, 216,3508; cds-WP_002901758.1, NZ_KK737546.1, 179251, 180297, +, 1047, 319982; cds-WP_002901761.1, NZ_KK737546.1, 180345, 180596, -, 252, 106630; cds-WP_002901763.1, NZ_KK737546.1, 181003, 183600, +, 2598, 1253282; cds-WP_002901772.1, NZ_KK737546.1, 183946, 184920, +, 975,136576; cds-WP_002901776.1, NZ_KK737546.1, 185166, 185333, +, 168, 33971; cds-WP_032429420.1, NZ_KK737546.1, 185722, 188400, +, 2679, 238620; cds-WP_032429421.1, NZ_KK737546.1, 188448, 189050, -, 603, 168913; cds-WP_002901779.1, NZ_KK737546.1, 189214, 189981, +, 768, 50501; cds-WP_002901780.1, NZ_KK737546.1, 190117, 190425, +, 309, 44487; cds-WP_002901781.1, NZ_KK737546.1, 190432, 191601, +, 1170, 81731; cds-WP_014907762.1, NZ_KK737546.1, 191793, 192530, +, 738, 29913; cds-WP_004210732.1, NZ_KK737546.1, 192530, 192856, +, 327, 68699; cds-WP_002901783.1, NZ_KK737546.1, 192988, 193206, -, 219, 317871; cds-WP_002901785.1, NZ_KK737546.1, 193482, 194231, -, 750, 269428; cds-WP_002901786.1, NZ_KK737546.1, 194303, 194482, -, 180, 54413; cds-WP_002901787.1, NZ_KK737546.1, 194641, 196575, -, 1935, 1455677; cds-WP_032421254.1, NZ_KK737546.1, 196657, 197814, -, 1158, 134855; cds-WP_004140277.1, NZ_KK737546.1, 198005, 198793, -, 789, 1099339; cds-WP_014907760.1, NZ_KK737546.1, 198992, 199534, -, 543, 7522; cds-WP_004151902.1, NZ_KK737546.1, 199782, 201161, +, 1380, 329024; cds-WP_004140269.1, NZ_KK737546.1, 201206, 202015, -, 810, 221164; cds-WP_004140266.1, NZ_KK737546.1, 202017, 203009, -, 993, 122273; cds-WP_004151901.1, NZ_KK737546.1, 203009, 203899, -, 891, 117599; cds-WP_032429422.1, NZ_KK737546.1, 204046, 205263, +, 1218, 29289; cds-WP_004892750.1, NZ_KK737546.1, 205484, 205723, -, 240, 65; cds-WP_016831904.1, NZ_KK737546.1, 205731, 206039, -, 309, 0; cds-WP_108918811.1, NZ_KK737546.1, 206036, 206677, -, 642, 427; cds-WP_016831906.1, NZ_KK737546.1, 206689, 206910, -, 222, 11; cds-WP_032426563.1, NZ_KK737546.1, 206907, 207434, -, 528, 0; cds-WP_008807811.1, NZ_KK737546.1, 207463, 208086, -, 624, 1435; cds-WP_032429423.1, NZ_KK737546.1, 208083, 208829, -, 747, 1091; cds-WP_008807813.1, NZ_KK737546.1, 208846, 209130, -, 285, 12826; cds-WP_008807814.1, NZ_KK737546.1, 209211, 209417, -, 207, 9017; cds-WP_161477215.1, NZ_KK737546.1, 209410, 209568, -, 159, 2930; cds-WP_004223168.1, NZ_KK737546.1, 209854, 210057, +, 204, 0; cds-WP_008807815.1, NZ_KK737546.1, 210087, 210734, -, 648, 785030; cds-WP_008807816.1, NZ_KK737546.1, 210731, 211165, -, 435, 338090; cds-WP_024622727.1, NZ_KK737546.1, 211385, 212074, -, 690, 226748; cds-WP_004178811.1, NZ_KK737546.1, 212179, 212412, +, 234, 2635; cds-WP_004141720.1, NZ_KK737546.1, 212452, 212772, +, 321, 34810; cds-WP_004151298.1, NZ_KK737546.1, 212859, 213005, +, 147, 1366; cds-WP_073545955.1, NZ_KK737546.1, 213046, 213897, +, 852, 48831; cds-WP_075209545.1, NZ_KK737546.1, 213902, 215317, +, 1416, 1615; cds-WP_032429424.1, NZ_KK737546.1, 215317, 215619, +, 303, 363; cds-AF52_RS21920, NZ_KK737546.1, 215616, 215912, +, 297, 7328; cds-WP_032429425.1, NZ_KK737546.1, 216035, 216298, +, 264, 51; cds-AF52_RS25000, NZ_KK737546.1, 216450, 216854, +, 405, 178; cds-WP_023286281.1, NZ_KK737546.1, 217078, 217299, +, 222, 35823; cds-WP_032429427.1, NZ_KK737546.1, 217303, 218010, +, 708, 234676; cds-WP_016831923.1, NZ_KK737546.1, 218000, 218224, +, 225, 0; cds-WP_032429428.1, NZ_KK737546.1, 218299, 218811, +, 513, 35756; cds-WP_032429704.1, NZ_KK737546.1, 218879, 219136, +, 258, 46853; cds-WP_032429429.1, NZ_KK737546.1, 219243, 219839, +, 597, 5754; cds-WP_004146340.1, NZ_KK737546.1, 220048, 220338, +, 291, 50; cds-WP_004177081.1, NZ_KK737546.1, 220335, 220697, +, 363, 0; cds-WP_023279926.1, NZ_KK737546.1, 220694, 220834, +, 141, 4; cds-WP_032429430.1, NZ_KK737546.1, 220831, 221520, +, 690, 0; cds-WP_024940884.1, NZ_KK737546.1, 222173, 222472, +, 300, 0; cds-WP_008807831.1, NZ_KK737546.1, 222469, 223008, +, 540, 3535; cds-WP_004190674.1, NZ_KK737546.1, 223005, 223349, +, 345, 2012; cds-WP_032429431.1, NZ_KK737546.1, 223346, 223621, +, 276, 143; cds-WP_004218558.1, NZ_KK737546.1, 224580, 224825, +, 246, 33190; cds-WP_013815099.1-5, NZ_KK737546.1, 225397, 226365, -, 969, 99238; cds-WP_032429432.1, NZ_KK737546.1, 226754, 227758, +, 1005, 122; cds-WP_004190663.1, NZ_KK737546.1, 227736, 229043, +, 1308, 2952; cds-AF52_RS21820, NZ_KK737546.1, 229043, 229582, +, 540, 0; cds-WP_170965341.1, NZ_KK737546.1, 229616, 229810, +, 195, 7491; cds-AF52_RS26695, NZ_KK737546.1, 231136, 231250, +, 115, 3133; cds-WP_187079184.1, NZ_KK737546.1, 231498, 232175, +, 678, 61; cds-WP_032429434.1, NZ_KK737546.1, 232159, 233271, +, 1113, 1274; cds-WP_032429435.1, NZ_KK737546.1, 233356, 234141, +, 786, 214; cds-WP_032429436.1, NZ_KK737546.1, 234152, 235105, +, 954, 103; cds-WP_142689607.1, NZ_KK737546.1, 235114, 235386, +, 273, 0; cds-WP_004217348.1, NZ_KK737546.1, 235427, 235822, +, 396, 232; cds-WP_004190649.1, NZ_KK737546.1, 235824, 236078, +, 255, 0; cds-WP_004217344.1, NZ_KK737546.1, 236307, 236690, +, 384, 0; cds-WP_008807839.1, NZ_KK737546.1, 236692, 237243, +, 552, 0; cds-WP_004190640.1, NZ_KK737546.1, 237240, 237632, +, 393, 3; cds-WP_004217341.1, NZ_KK737546.1, 237656, 238828, +, 1173,7702; cds-WP_004226994.1, NZ_KK737546.1, 238882, 239364, +, 483, 104; cds-WP_074185736.1, NZ_KK737546.1, 239502, 239696, +, 195, 2516; cds-WP_032429437.1, NZ_KK737546.1, 240136, 240315, +, 180, 162231; cds-WP_032417044.1, NZ_KK737546.1, 240325, 240810, +, 486, 863671; cds-AF52_RS21755, NZ_KK737546.1, 240849, 241094, -, 246, 152574; cds-WP_013815099.1-6, NZ_KK737546.1, 241160, 242128, -, 969, 98171; cds-WP_032429438.1, NZ_KK737546.1, 242146, 242397, -, 252, 6591; cds-WP_077254381.1, NZ_KK737546.1, 242561, 242902, +, 342, 4335; cds-AF52_RS21745, NZ_KK737546.1, 243004, 244416, +, 1413, 2199; cds-WP_013815099.1-7, NZ_KK737546.1, 244471, 245439, -, 969, 82493; cds-AF52_RS21735, NZ_KK737546.1, 245474, 246952, +, 1479, 39062; cds-WP_032429439.1, NZ_KK737546.1, 246952, 247425, +, 474, 0; cds-WP_032429440.1, NZ_KK737546.1, 247412, 247894, +, 483, 0; cds-WP_032429442.1, NZ_KK737546.1, 247904, 248284, +, 381, 15997; cds-AF52_RS21715, NZ_KK737546.1, 248281, 251259, +, 2979, 30396; cds-WP_013815099.1-8, NZ_KK737546.1, 251293, 252261, +, 969, 99011; cds-AF52_RS0123075, NZ_KK737546.1, 252965, 253330, -, 366, 26349; cds-WP_032429443.1, NZ_KK737546.1, 253422, 253970, +, 549, 4707; cds-WP_004179627.1, NZ_KK737546.1, 255020, 255439, +, 420, 13336; cds-AF52_RS0123080, NZ_KK737546.1, 255441, 256706, +, 1266, 32979; cds-WP_032429444.1, NZ_KK737546.1, 256748, 257389, +, 642, 91007; cds-WP_016831214.1, NZ_KK737546.1, 257382, 258056, -, 675, 9565; cds-WP_002901816.1, NZ_KK737546.1, 258261, 259226, -, 966, 154422; cds-WP_004140258.1, NZ_KK737546.1, 259223, 260866, -, 1644, 209872; cds-WP_021441607.1, NZ_KK737546.1, 261200, 262108, -, 909, 669; cds-WP_019705465.1, NZ_KK737546.1, 262216, 263046, +, 831, 29646; cds-WP_032429447.1, NZ_KK737546.1, 263025, 265334, -, 2310, 157256; cds-WP_002901900.1, NZ_KK737546.1, 265506, 266675, +, 1170, 152; cds-WP_021441611.1, NZ_KK737546.1, 266701, 268092, +, 1392, 9869; cds-WP_004148137.1, NZ_KK737546.1, 268083, 269072, -, 990, 24472; cds-WP_002901908.1, NZ_KK737546.1, 269225, 269893, +, 669, 292569; cds-WP_002901911.1, NZ_KK737546.1, 269949, 270173, +, 225, 252309; cds-WP_002901913.1, NZ_KK737546.1, 270173, 270532, +, 360, 119496; cds-WP_002901915.1, NZ_KK737546.1, 270561, 270779, +, 219, 2003; cds-WP_002901917.1, NZ_KK737546.1, 270882, 272279, +, 1398, 134373; cds-WP_004140239.1, NZ_KK737546.1, 272276, 273337, +, 1062, 50437; cds-WP_004888583.1, NZ_KK737546.1, 273427, 274326, -, 900, 3925; cds-WP_002901977.1, NZ_KK737546.1, 274440, 275753, +, 1314, 3573; cds-WP_002901979.1, NZ_KK737546.1, 275926, 277467, +, 1542, 83329; cds-WP_032428836.1, NZ_KK737546.1, 277569, 278621, +, 1053, 57350; cds-WP_004152131.1, NZ_KK737546.1, 278692, 279198, -, 507, 741292; cds-WP_032429452.1, NZ_KK737546.1, 279301, 280266, +, 966, 27056; cds-WP_004152134.1, NZ_KK737546.1, 280263, 280970, -, 708, 13879; cds-WP_004148146.1, NZ_KK737546.1, 281043, 281159, +, 117, 2958; cds-WP_004176446.1, NZ_KK737546.1, 281156, 282772, +, 1617, 369024; cds-WP_021441615.1, NZ_KK737546.1, 282873, 283259, -, 387, 151724; cds-WP_004190572.1, NZ_KK737546.1, 283490, 284323, +, 834, 43661; cds-WP_004152138.1, NZ_KK737546.1, 284448, 285332, -, 885, 23307; cds-WP_004190569.1, NZ_KK737546.1, 285505, 286641, +, 1137, 949; cds-WP_023287551.1, NZ_KK737546.1, 286714, 287916, +, 1203, 132; cds-WP_004152141.1, NZ_KK737546.1, 287995, 288864, +, 870, 1309; cds-AF52_RS21545, NZ_KK737546.1, 289095, 289340, +, 246, 0; cds-WP_072001575.1, NZ_KK737546.1, 289412, 289558, -, 147, 5011; cds-WP_013815099.1-9, NZ_KK737546.1, 289602, 290570, -, 969, 89107; cds-WP_187079185.1, NZ_KK737546.1, 290586, 291959, +, 1374, 14514; cds-WP_002902422.1, NZ_KK737546.1, 292005, 292940, -, 936, 493171; cds-WP_004140161.1, NZ_KK737546.1, 293174, 293608, -, 435, 145283; cds-WP_002902432.1, NZ_KK737546.1, 293690, 293902, -, 213, 2224; cds-WP_004176397.1, NZ_KK737546.1, 294046, 295170, -, 1125, 32922; cds-WP_021313170.1, NZ_KK737546.1, 295527, 299054, -, 3528, 210661; cds-WP_004140155.1, NZ_KK737546.1, 299417, 299680, +, 264, 23863; cds-WP_032429459.1, NZ_KK737546.1, 299726, 300166, -, 441, 54064; cds-WP_002902515.1, NZ_KK737546.1, 300289, 301278, -, 990, 414594; cds-WP_002902516.1, NZ_KK737546.1, 301426, 301926, -, 501, 42470; cds-WP_023279029.1, NZ_KK737546.1, 302133, 304772, +, 2640, 120215; cds-WP_004140145.1, NZ_KK737546.1, 304769, 304954, +, 186, 16264; cds-WP_002902522.1, NZ_KK737546.1, 304962, 305285, +, 324, 184882; cds-WP_004140140.1, NZ_KK737546.1, 305286, 306188, -, 903, 92140; cds-WP_032429462.1, NZ_KK737546.1, 306424, 307923, +, 1500, 7768; cds-WP_032429463.1, NZ_KK737546.1, 308048, 308938, +, 891, 12248; cds-WP_021440033.1, NZ_KK737546.1, 308982, 309965, +, 984, 47459; cds-WP_021440034.1, NZ_KK737546.1, 309968, 312235, -, 2268, 582001; cds-WP_032429464.1, NZ_KK737546.1, 312712, 314757, -, 2046, 1686240; cds-WP_032428268.1, NZ_KK737546.1, 315044, 315973, +, 930, 949070; cds-WP_032429465.1, NZ_KK737546.1, 315985, 316272, +, 288, 143281; cds-WP_020324632.1, NZ_KK737546.1, 316280, 317035, +, 756, 804337; cds-WP_021440037.1, NZ_KK737546.1, 317047, 317544, +, 498, 608321; cds-WP_002902772.1, NZ_KK737546.1, 317552, 318622, +, 1071, 733391; cds-WP_032429468.1, NZ_KK737546.1, 318619, 319386, +, 768, 386246; cds-WP_004140117.1,
NZ_KK737546.1, 319389, 320177, +, 789, 143805; cds-WP_021440038.1, NZ_KK737546.1, 320178, 321602, +, 1425, 167894; cds-WP_002902876.1, NZ_KK737546.1, 321592, 322014, +, 423, 222067; cds-WP_014907683.1, NZ_KK737546.1, 322014, 323219, +, 1206,162404; cds-WP_002902877.1, NZ_KK737546.1, 323246, 324562, +, 1317, 153448; cds-WP_004194367.1, NZ_KK737546.1, 324682, 325608, +, 927, 277575; cds-WP_002902883.1, NZ_KK737546.1, 325618, 326214, +, 597, 57510; cds-WP_024940864.1, NZ_KK737546.1, 326223, 326633, +, 411, 12266; cds-WP_002902889.1, NZ_KK737546.1, 326670, 327275, -, 606, 56045; cds-WP_032429470.1, NZ_KK737546.1, 327480, 331382, +, 3903, 475495; cds-WP_020324670.1, NZ_KK737546.1, 331443, 332117, -, 675, 102437; cds-WP_021440039.1, NZ_KK737546.1, 332118, 333497, -, 1380, 55747; cds-WP_002902965.1, NZ_KK737546.1, 333494, 334579, -, 1086, 30121; cds-WP_002902967.1, NZ_KK737546.1, 334608, 336260, -, 1653, 133664; cds-WP_002902969.1, NZ_KK737546.1, 336260, 336691, -, 432, 8782; cds-WP_002902971.1, NZ_KK737546.1, 336799, 337746, -, 948, 54733; cds-WP_032429472.1, NZ_KK737546.1, 337847, 338845, +, 999, 228685; cds-WP_004151574.1, NZ_KK737546.1, 339001, 340275, -, 1275, 5660305; cds-WP_004224524.1, NZ_KK737546.1, 340488, 340634, -, 147, 43323; cds-WP_020802294.1, NZ_KK737546.1, 340804, 342009, +, 1206, 2066639; cds-WP_004140076.1, NZ_KK737546.1, 342028, 343050, +, 1023, 939181; cds-WP_004183870.1, NZ_KK737546.1, 343041, 344507, +, 1467, 861982; cds-WP_004190366.1, NZ_KK737546.1, 344529, 345869, +, 1341, 389975; cds-WP_004176358.1, NZ_KK737546.1, 345880, 346875, +, 996, 202344; cds-WP_016947259.1, NZ_KK737546.1, 346885, 348315, +, 1431, 272379; cds-WP_021440044.1, NZ_KK737546.1, 348524, 349849, -, 1326, 43019; cds-WP_020316474.1, NZ_KK737546.1, 350230, 351030, +, 801, 380; cds-WP_004140057.1, NZ_KK737546.1, 351092, 351229, -, 138, 581; cds-WP_004151568.1, NZ_KK737546.1, 351228, 352667, +, 1440, 10582; cds-WP_002903227.1, NZ_KK737546.1, 352716, 353714, -, 999, 11405; cds-WP_002903230.1, NZ_KK737546.1, 353997, 354155, +, 159, 359; cds-WP_004224558.1, NZ_KK737546.1, 354190, 354582, -, 393, 4; cds-WP_002903233.1, NZ_KK737546.1, 354833, 355066, -, 234, 0; cds-WP_021440045.1, NZ_KK737546.1, 355063, 356271, -, 1209, 36369; cds-WP_004179665.1, NZ_KK737546.1, 356375, 356728, -, 354, 9178; cds-WP_004179667.1, NZ_KK737546.1, 356965, 357444, +, 480, 157; cds-WP_004151566.1, NZ_KK737546.1, 357514, 357717, -, 204, 0; cds-WP_032429476.1, NZ_KK737546.1, 358029, 359057, +, 1029, 14117; cds-WP_032429478.1, NZ_KK737546.1, 359071, 360243, -, 1173, 254279; cds-WP_021440047.1, NZ_KK737546.1, 360295, 361887, -, 1593, 333578; cds-WP_021440048.1, NZ_KK737546.1, 362060, 363088, +, 1029, 60391; cds-WP_004190339.1, NZ_KK737546.1, 363128, 364687, -, 1560, 301714; cds-WP_032429480.1, NZ_KK737546.1, 364774, 365952, -, 1179, 627504; cds-WP_032429482.1, NZ_KK737546.1, 366154, 367800, +, 1647, 6342208; cds-WP_002903315.1, NZ_KK737546.1, 368140, 369540, +, 1401, 355650; cds-WP_021440051.1, NZ_KK737546.1, 369530, 370462, -, 933, 37806; cds-WP_021440052.1, NZ_KK737546.1, 370538, 371839, -, 1302, 13810; cds-WP_032429484.1, NZ_KK737546.1, 372095, 372457, -, 363, 1873; cds-WP_002903377.1, NZ_KK737546.1, 372700, 373419, -, 720, 32285; cds-WP_002903379.1, NZ_KK737546.1, 373548, 373883, +, 336, 8193; cds-WP_004179681.1, NZ_KK737546.1, 373880, 374602, -, 723, 179716; cds-WP_004183892.1, NZ_KK737546.1, 374639, 376021, -, 1383, 87748; cds-WP_002903384.1, NZ_KK737546.1, 376199, 377149, -, 951, 335909; cds-WP_004140012.1, NZ_KK737546.1, 377146, 377328, -, 183, 5390; cds-WP_032422490.1, NZ_KK737546.1, 377671, 379200, +, 1530, 342550; cds-WP_002903386.1, NZ_KK737546.1, 379211, 380599, +, 1389, 179783; cds-WP_004887037.1, NZ_KK737546.1, 380748, 380936, +, 189, 43255; cds-WP_002903391.1, NZ_KK737546.1, 381046, 381996, -, 951, 342137; cds-WP_002903394.1, NZ_KK737546.1, 382135, 382887, -, 753, 587250; cds-WP_004224598.1, NZ_KK737546.1, 383082, 383597, -, 516, 20280; cds-WP_002903398.1, NZ_KK737546.1, 383890, 384048, +, 159, 90; cds-AF52_RS21120, NZ_KK737546.1, 384081, 384533, -, 453, 0; cds-WP_023343160.1, NZ_KK737546.1, 384656, 384919, -, 264, 130; cds-WP_032429712.1, NZ_KK737546.1, 384927, 385493, -, 567, 770; cds-WP_072001561.1, NZ_KK737546.1, 385495, 386064, -, 570, 1861; cds-WP_032429489.1, NZ_KK737546.1, 386061, 386354, -, 294, 4393; cds-AF52_RS0123125, NZ_KK737546.1, 386354, 386536, -, 183, 5456; cds-WP_032429490.1, NZ_KK737546.1, 386715, 386915, -, 201, 3626; cds-WP_032429491.1, NZ_KK737546.1, 386912, 387394, -, 483, 39; cds-AF52_RS26290, NZ_KK737546.1, 387508, 387846, -, 339, 0; cds-WP_032429493.1, NZ_KK737546.1, 388073, 388858, -, 786, 0; cds-WP_023343167.1, NZ_KK737546.1, 388851, 389051, -, 201, 0; cds-WP_032429494.1, NZ_KK737546.1, 389051, 389578, -, 528, 27; cds-WP_032429495.1, NZ_KK737546.1, 389714, 390544, -, 831, 4488; cds-WP_032429496.1, NZ_KK737546.1, 390597, 390968, -, 372, 68865; cds-WP_187079182.1, NZ_KK737546.1, 391972, 392158, +, 187, 9330; cds-AF52_RS26730, NZ_KK737546.1, 393389, 393527, +, 139, 45938; cds-WP_032429497.1, NZ_KK737546.1, 393786, 394019, -, 234, 4277; cds-WP_032429029.1, NZ_KK737546.1, 394208, 394867, -, 660, 61280; cds-WP_023343174.1, NZ_KK737546.1, 394955, 395155, +, 201, 0; cds-WP_032429498.1, NZ_KK737546.1, 395200, 395751, +, 552, 744; cds-WP_023322342.1, NZ_KK737546.1, 395924, 396103, +, 180, 0; cds-AF52_RS21035, NZ_KK737546.1, 396093, 396482, +, 390, 8; cds-WP_013815099.1-10, NZ_KK737546.1, 396540, 397508, -, 969, 107577; cds-AF52_RS21025, NZ_KK737546.1, 397537, 398793, -, 1257, 28096; cds-WP_004143402.1, NZ_KK737546.1, 399073, 399846, -, 774, 119895; cds-WP_023301931.1, NZ_KK737546.1, 399893, 401398, -, 1506, 29878; cds-WP_021440130.1, NZ_KK737546.1, 401452, 402201, -, 750, 151475; cds-WP_021440129.1, NZ_KK737546.1, 402565, 403365, +, 801, 178; cds-AF52_RS21000, NZ_KK737546.1, 403475, 404163, +, 689, 15691; cds-WP_032429499.1, NZ_KK737546.1, 404175, 405119, +, 945, 26155; cds-WP_004143414.1, NZ_KK737546.1, 405156, 406490, +, 1335, 117154; cds-WP_004176179.1, NZ_KK737546.1, 406492, 406881, +, 390, 95; cds-WP_021440127.1, NZ_KK737546.1, 407130, 408686, +, 1557, 63390; cds-WP_021440126.1, NZ_KK737546.1, 408683, 410275, +, 1593, 106246; cds-WP_004190119.1, NZ_KK737546.1, 410287, 411021, +, 735, 669; cds-WP_004148393.1, NZ_KK737546.1, 411033, 412223, +, 1191, 58910; cds-WP_004143425.1, NZ_KK737546.1, 412359, 413696, +, 1338, 8975; cds-WP_004148392.1, NZ_KK737546.1, 413710, 414588, +, 879, 7914; cds-WP_004176181.1, NZ_KK737546.1, 414665, 416035, +, 1371, 3065; cds-WP_021440125.1, NZ_KK737546.1, 416049, 417224, +, 1176, 213; cds-WP_002904834.1, NZ_KK737546.1, 417221, 418378, +, 1158, 50567; cds-WP_002904832.1, NZ_KK737546.1, 418461, 419336, -, 876, 27012; cds-WP_021440123.1, NZ_KK737546.1, 419448, 420332, -, 885, 23669; cds-WP_002904827.1, NZ_KK737546.1, 420444, 421187, +, 744, 4054; cds-WP_002904825.1, NZ_KK737546.1, 421193, 421891, -, 699, 3622; cds-WP_032417116.1, NZ_KK737546.1, 422159, 423691, +, 1533, 4204; cds-WP_021440120.1, NZ_KK737546.1, 423703, 425031, +, 1329, 35078; cds-WP_032417115.1, NZ_KK737546.1, 425777, 427222, +, 1446, 1281378; cds-WP_021440118.1, NZ_KK737546.1, 427212, 427655, +, 444, 475209; cds-WP_002904816.1, NZ_KK737546.1, 428072, 428311, +, 240, 2133; cds-WP_009308870.1, NZ_KK737546.1, 428336, 428476, +, 141, 0; cds-WP_004148380.1, NZ_KK737546.1, 428664, 429599, +, 936, 14203; cds-WP_004143447.1, NZ_KK737546.1, 429633, 431288, -, 1656, 30112; cds-WP_002904810.1, NZ_KK737546.1, 431585, 431833, +, 249, 20038; cds-WP_002904808.1, NZ_KK737546.1, 432123, 432341, +, 219, 26530; cds-WP_004179825.1, NZ_KK737546.1, 432322, 433104, +, 783, 92944; cds-WP_004176193.1, NZ_KK737546.1, 433331, 433618, -, 288, 194; cds-WP_004143457.1, NZ_KK737546.1, 434062, 434625, +, 564, 0; cds-WP_004176194.1, NZ_KK737546.1, 434703, 435398, +, 696, 228; cds-WP_016831617.1, NZ_KK737546.1, 435414, 437915, +, 2502, 430; cds-WP_004176196.1, NZ_KK737546.1, 437903, 438907, +, 1005, 171; cds-WP_002904794.1, NZ_KK737546.1, 439049, 440341, +, 1293, 125753; cds-WP_004176197.1, NZ_KK737546.1, 440358, 441680, -, 1323, 48217; cds-WP_032417111.1, NZ_KK737546.1, 441680, 441946, -, 267, 19079; cds-WP_002904788.1, NZ_KK737546.1, 442211, 442537, -, 327, 3017; cds-WP_002904787.1, NZ_KK737546.1, 442534, 443034, -, 501, 35959; cds-WP_032429500.1, NZ_KK737546.1, 443012, 444391, -, 1380, 36567; cds-WP_002904783.1, NZ_KK737546.1, 444737, 445891, +, 1155, 32; cds-WP_032429501.1, NZ_KK737546.1, 445872, 446795, -, 924, 496; cds-WP_021440114.1, NZ_KK737546.1, 446921, 447958, +, 1038, 55144; cds-WP_032429502.1, NZ_KK737546.1, 447970, 448953, +, 984, 114418; cds-WP_014907616.1, NZ_KK737546.1, 448930, 450168, -, 1239, 4; cds-WP_032429503.1, NZ_KK737546.1, 450274, 451218, +, 945, 35801; cds-WP_032429504.1, NZ_KK737546.1, 451228, 451983, -, 756, 2851; cds-WP_002904646.1, NZ_KK737546.1, 452160, 452471, -, 312, 57; cds-WP_002904643.1, NZ_KK737546.1, 452493, 452756, -, 264, 40789; cds-WP_004176215.1, NZ_KK737546.1, 453039, 454067, +, 1029, 24; cds-WP_002904638.1, NZ_KK737546.1, 454091, 454897, +, 807, 4620; cds-WP_021440110.1, NZ_KK737546.1, 454894, 455685, +, 792, 28623; cds-WP_021440109.1, NZ_KK737546.1, 455682, 456341, +, 660, 122; cds-WP_021440108.1, NZ_KK737546.1, 456352, 457425, -, 1074, 9740; cds-WP_004143508.1, NZ_KK737546.1, 457500, 458345, +, 846, 57923; cds-WP_032417109.1, NZ_KK737546.1, 458342, 459499, -, 1158, 2253; cds-WP_032417108.1, NZ_KK737546.1, 459707, 460129, +, 423, 28561; cds-WP_004176220.1, NZ_KK737546.1, 460285, 461472, +, 1188, 56409; cds-WP_002904624.1, NZ_KK737546.1, 461701, 461991, +, 291, 10309; cds-WP_004176221.1, NZ_KK737546.1, 462200, 462961, +, 762, 7413; cds-WP_004143517.1, NZ_KK737546.1, 463122, 463442, +, 321, 373; cds-WP_032429505.1, NZ_KK737546.1, 463433, 463813, +, 381, 23087; cds-WP_021440101.1, NZ_KK737546.1, 464024, 464470, -, 447, 80666; cds-WP_004183995.1, NZ_KK737546.1, 464724, 466175, -, 1452, 195298; cds-WP_021440100.1, NZ_KK737546.1, 466289, 466819, -, 531, 31486; cds-WP_004148352.1, NZ_KK737546.1, 466897, 468321, -, 1425, 484159; cds-WP_004190206.1, NZ_KK737546.1, 468436, 468804, -, 369, 1736; cds-AF52_RS0123140, NZ_KK737546.1, 468804, 469730, -, 927, 1485; cds-WP_021440098.1, NZ_KK737546.1, 469811, 471199, -, 1389, 2430; cds-WP_002904495.1, NZ_KK737546.1, 471301, 472179, +, 879, 9398; cds-WP_009308907.1, NZ_KK737546.1, 472133, 472615, -, 483, 8842; cds-WP_021440097.1, NZ_KK737546.1, 472760, 473704, +, 945, 86178; cds-WP_002904489.1, NZ_KK737546.1, 473719, 474480, -, 762, 263545; cds-WP_016532445.1, NZ_KK737546.1, 474482, 475468, -, 987, 10377; cds-WP_032429506.1, NZ_KK737546.1, 475461, 476696, -, 1236, 44; cds-WP_004179774.1, NZ_KK737546.1, 476871, 478019, +, 1149, 22498; cds-WP_021440094.1, NZ_KK737546.1, 478034, 478918, +, 885, 3859; cds-WP_021440093.1, NZ_KK737546.1, 479004, 479954, +, 951, 22435; cds-WP_004179768.1, NZ_KK737546.1, 480018, 481337, +, 1320, 79551; cds-WP_004179766.1, NZ_KK737546.1, 481383, 482177, -, 795, 67966; cds-WP_002904407.1, NZ_KK737546.1, 482379, 483578, +, 1200, 47204; cds-WP_002904403.1, NZ_KK737546.1, 483647, 484315, -, 669, 73116; cds-WP_004217987.1, NZ_KK737546.1, 484359, 484487, -, 129, 1259; cds-WP_013815099.1-11, NZ_KK737546.1, 484550, 485518, -, 969, 73302; cds-WP_032429507.1, NZ_KK737546.1, 485643, 486077, +, 435, 4439; cds-WP_002904397.1, NZ_KK737546.1, 486098, 486472, +, 375, 145401; cds-WP_002904394.1, NZ_KK737546.1, 486513, 486731, +, 219,88400; cds-WP_004143566.1, NZ_KK737546.1, 486780, 487679, -, 900, 35707; cds-WP_021440091.1, NZ_KK737546.1, 487875, 489071, +, 1197, 13816; cds-WP_002904389.1, NZ_KK737546.1, 489201, 489683, +, 483, 31; cds-WP_004176246.1, NZ_KK737546.1, 489719, 490417, -, 699, 69; cds-WP_004148342.1, NZ_KK737546.1, 490427, 491224, -, 798, 1; cds-WP_032429508.1, NZ_KK737546.1, 491224, 492297, -, 1074, 53; cds-WP_015958384.1, NZ_KK737546.1, 492297, 493871, -, 1575, 36; cds-WP_032429509.1, NZ_KK737546.1, 493933, 495204, -, 1272, 0; cds-WP_004148338.1, NZ_KK737546.1, 495376, 496479, -, 1104, 7121; cds-WP_004183961.1, NZ_KK737546.1, 496628, 497026, -, 399, 0; cds-WP_029602386.1, NZ_KK737546.1, 497094, 498191, +, 1098, 1847; cds-WP_004205985.1, NZ_KK737546.1, 498160, 498375, +, 216, 83683; cds-WP_021440087.1, NZ_KK737546.1, 498428, 498868, -, 441, 120445; cds-WP_004183956.1, NZ_KK737546.1, 499122, 500186, -, 1065, 1082;
cds-WP _021440086.1, NZ_KK737546.1, 500383, 503490, +, 3108, 28210; cds-WP_032429510.1, NZ_KK737546.1, 503545, 504810, +, 1266, 256634; cds-WP _021440085.1, NZ_KK737546.1, 504841, 505929, -, 1089, 64022; cds-WP_004176262.1, NZ_KK737546.1, 506016, 506276, -, 261, 39274; cds-WP_021440083.1, NZ_KK737546.1, 506568, 507434, +, 867, 25979; cds-WP_002210513.1, NZ_KK737546.1, 507455, 508216, -, 762, 58752; cds-AF52_RS0123150, NZ_KK737546.1, 508478, 509380, +, 903, 23137; cds-WP_004224682.1, NZ_KK737546.1, 509392, 510657, +, 1266, 73032; cds-WP _002210516.1, NZ_KK737546.1, 510650, 511270, +, 621, 6456; cds-WP _004176276.1, NZ_KK737546.1, 511275, 512057, +, 783, 20802; cds-WP _013815099.1-12, NZ_KK737546.1, 512157, 513125, -, 969, 92834; cds-AF52_RS0123155, NZ_KK737546.1, 513181, 514545, +, 1365, 2935; cds-WP _021440081.1, NZ_KK737546.1, 514572, 515498, -, 927, 55422; cds-AF52_RS0123160, NZ_KK737546.1, 515809, 516421, +, 613, 7; cds-WP_004195995.1, NZ_KK737546.1, 516452, 516571, -, 120, 0; cds-WP_004143607.1, NZ_KK737546.1, 516972, 518189, +, 1218, 36209; cds-WP_032429511.1, NZ_KK737546.1, 518210, 518722, -, 513, 4599; cds-WP_004183942.1, NZ_KK737546.1, 518888, 519361, -, 474, 285438; cds-WP _004173721.1, NZ_KK737546.1, 519807, 519941, -, 135, 11024; cds-WP_020316505.1, NZ_KK737546.1, 520183, 520305, +, 123, 0; cds-WP _013815099.1-13, NZ_KK737546.1, 520461, 521429, +, 969, 92525; cds-WP _032429512.1, NZ_KK737546.1, 521466, 522074, +, 609, 76962; cds-WP _004143611.1, NZ_KK737546.1, 522071, 522193, -, 123, 0; cds-WP_004219446.1, NZ_KK737546.1, 522248, 522376, -, 129, 0; cds-WP _004176282.1, NZ_KK737546.1, 522548, 523222, +, 675, 0; cds-WP _015958377.1, NZ_KK737546.1, 523273, 524115, -, 843, 55035; cds-WP _002903735.1, NZ_KK737546.1, 524143, 524529, +, 387, 1394; cds-WP _032417100.1, NZ_KK737546.1, 524607, 526652, -, 2046, 26183; cds-WP_032429513.1, NZ_KK737546.1, 526783, 527532, +, 750, 361122; cds-WP _002903730.1, NZ_KK737546.1, 527624, 528310, +, 687, 173988; cds-WP _004183938.1, NZ_KK737546.1, 528363, 528794, -, 432, 106855; cds-WP_032429514.1, NZ_KK737546.1, 529059, 530522, -, 1464, 9389; cds-WP_032429515.1, NZ_KK737546.1, 530776, 532059, -, 1284, 41224; cds-WP _072143281.1, NZ_KK737546.1, 532173, 532535, -, 363, 8384; cds-WP _002903720.1, NZ_KK737546.1, 532644, 532985, +, 342, 163336; cds-WP_002903719.1, NZ_KK737546.1, 533064, 533624, +, 561, 316122; cds-WP_032428279.1, NZ_KK737546.1, 533618, 534328, -, 711, 96596; cds-WP _002903714.1, NZ_KK737546.1, 534430, 534699, +, 270, 158610; cds-WP_032429516.1, NZ_KK737546.1, 534850, 537285, +, 2436, 35850; cds-WP _004152235.1, NZ_KK737546.1, 537296, 537913, +, 618, 10483; cds-WP _002903710.1, NZ_KK737546.1, 537915, 538772, +, 858, 1481; cds-WP_004209765.1, NZ_KK737546.1, 538814, 539422, +, 609, 11771; cds-WP _021440073.1, NZ_KK737546.1, 539556, 540851, +, 1296, 58846; cds-WP _032428278.1, NZ_KK737546.1, 540804, 541499, -, 696, 46070; cds-WP _002903701.1, NZ_KK737546.1, 541627, 542847, -, 1221, 394131; cds-WP_002903700.1, NZ_KK737546.1, 542971, 543873, -, 903, 2529; cds-WP _002903698.1, NZ_KK737546.1, 544004, 545254, +, 1251, 31956; cds-WP _029884324.1, NZ_KK737546.1, 545594, 547081, +, 1488, 298071; cds-WP _002903693.1, NZ_KK737546.1, 547246, 547665, +, 420, 36423; cds-WP _013815099.1-14, NZ_KK737546.1, 548708, 549676, -, 969, 75382; cds-WP _050484275.1, NZ_KK737546.1, 549709, 550482, +, 774, 44851; cds-WP_004224649.1, NZ_KK737546.1, 550496, 550612, +, 117, 17570; cds-WP _032429518.1, NZ_KK737546.1, 550656, 550985, -, 330, 91899; cds-WP_002903681.1, NZ_KK737546.1, 550972, 551334, -, 363, 570420; cds-WP_002903679.1, NZ_KK737546.1, 551777, 552811, +, 1035, 79626; cds-WP _004176297.1, NZ_KK737546.1, 553036, 554691, +, 1656, 0; cds-WP_009486289.1, NZ_KK737546.1, 554691, 555533, +, 843, 90; cds-WP _032428276.1, NZ_KK737546.1, 555551, 555850, +, 300, 0; cds-WP _004143105.1, NZ_KK737546.1, 555843, 556676, +, 834, 538; cds-WP_032429519.1, NZ_KK737546.1, 556676, 557476, +, 801, 0; cds-WP _004148291.1, NZ_KK737546.1, 557613, 558572, +, 960, 1589; cds-WP _004152239.1, NZ_KK737546.1, 558576, 559193, +, 618, 1173; cds-WP _021440066.1, NZ_KK737546.1, 559193, 560095, +, 903, 34; cds-WP _002903632.1, NZ_KK737546.1, 560085, 561011, -, 927, 129412; cds-AF52_RS20250, NZ_KK737546.1, 561169, 562206, -, 1038, 4231; cds-AF52_RS20245, NZ_KK737546.1, 562264, 562518, -, 255, 23083; cds-WP_032429520.1, NZ_KK737546.1, 563260, 563512, -, 253, 18095; cds-AF52_RS20235, NZ_KK737546.1, 563544, 564125, -, 582, 12; cds-WP_032428274.1, NZ_KK737546.1, 564433, 565356, -, 924, 75938; cds-WP_016529947.1, NZ_KK737546.1, 565520, 565876, -, 357, 990; cds-WP _032429521.1, NZ_KK737546.1, 566032, 567648, +, 1617, 149691; cds-WP _004176303.1, NZ_KK737546.1, 567645, 568364, +, 720, 91521; cds-WP _020803870.1, NZ_KK737546.1, 568345, 569295, -, 951, 8146; cds-WP _004152245.1, NZ_KK737546.1, 569363, 572140, -, 2778, 22243; cds-WP_004148278.1, NZ_KK737546.1, 572782, 574293, +, 1512, 64; cds-WP _004152246.1, NZ_KK737546.1, 574348, 576000, +, 1653, 27194; cds-WP _032417097.1, NZ_KK737546.1, 576164, 577780, +, 1617, 8790; cds-WP_004143067.1, NZ_KK737546.1, 578370, 578759, -, 390, 3061; cds-WP_004151556.1, NZ_KK737546.1, 578752, 579516, -, 765, 25994; cds-WP_032429522.1, NZ_KK737546.1, 579506, 580858, -, 1353, 8882; cds-WP _009308944.1, NZ_KK737546.1, 580868, 582070, -, 1203, 700; cds-WP _032429523.1, NZ_KK737546.1, 582081, 582737, -, 657, 29; cds-WP_002903522.1, NZ_KK737546.1, 582748, 583434, -, 687, 27064; cds-WP_004151560.1, NZ_KK737546.1, 583604, 584410, +, 807, 27428; cds-WP _032429524.1, NZ_KK737546.1, 584407, 584970, -, 564, 41664; cds-WP _002903460.1, NZ_KK737546.1, 585072, 585980, -, 909, 47486; cds-WP_021440059.1, NZ_KK737546.1, 586147, 587457, +, 1311, 97543; cds-WP_020805029.1, NZ_KK737546.1, 587457, 588902, +, 1446, 412512; cds-WP_017879817.1, NZ_KK737546.1, 589022, 590140, +, 1119, 67305; cds-WP _072001564.1, NZ_KK737546.1, 590269, 591363, +, 1095, 251793; cds-WP _072001565.1, NZ_KK737546.1, 591373, 592209, +, 837, 114369; cds-WP _021314545.1, NZ_KK737546.1, 592340, 592711, -, 372, 508964; cds-WP _032429525.1, NZ_KK737546.1, 593013, 593564, -, 552, 1629; cds-AF52_RS27770, NZ_KK737546.1, 593656, 594012, +, 357, 0; cds-WP_013815099.1-15, NZ_KK737546.1, 596036, 597004, -, 969, 91647; cds-AF52_RS25145, NZ_KK737546.1, 597030, 597290, +, 261, 6878; cds-WP _004150800.1, NZ_KK737546.1, 597408, 598658, -, 1251, 8405681; cds-WP _004150801.1, NZ_KK737546.1, 598899, 599549, +, 651, 92532; cds-WP _021313538.1, NZ_KK737546.1, 599566, 600024, +, 459, 108442; cds-WP _004150803.1, NZ_KK737546.1, 600081, 601187, +, 1107, 1072857; cds-WP _004140557.1, NZ_KK737546.1, 601242, 601883, +, 642, 995954; cds-WP_004176557.1, NZ_KK737546.1, 601887, 603257, +, 1371, 1471179; cds-WP_004213084.1, NZ_KK737546.1, 603312, 603674, -, 363, 42374; cds-WP _004183655.1, NZ_KK737546.1, 603758, 604564, -, 807, 2556; cds-WP _004150807.1, NZ_KK737546.1, 604848, 605519, +, 672, 266724; cds-WP_004147969.1, NZ_KK737546.1, 605519, 606985, +, 1467, 417181; cds-WP_004176560.1, NZ_KK737546.1, 607071, 608192, +, 1122, 370138; cds-WP _004150810.1, NZ_KK737546.1, 608332, 609564, -, 1233, 1529; cds-WP_004150811.1, NZ_KK737546.1, 609823, 610959, +, 1137, 105522; cds-WP _004150812.1, NZ_KK737546.1, 610943, 611800, +, 858, 98488; cds-WP _004150813.1, NZ_KK737546.1, 611797, 612582, +, 786, 11628; cds-WP _004140590.1, NZ_KK737546.1, 612579, 613625, +, 1047, 371271; cds-WP_032429526.1, NZ_KK737546.1, 613672, 615396, -, 1725, 64823; cds-WP _032429527.1, NZ_KK737546.1, 615393, 617021, -, 1629, 4749; cds-WP_032429528.1, NZ_KK737546.1, 617259, 619262, -, 2004, 3176901; cds-WP_009484250.1, NZ_KK737546.1, 619282, 620235, -, 954, 10562; cds-AF52_RS0123220, NZ_KK737546.1, 620263, 620421, -, 159, 37058; cds-AF52_RS0123225, NZ_KK737546.1, 620476, 621173, +, 698, 374498; cds-WP _032429529.1, NZ_KK737546.1, 621229, 622248, -, 1020, 0; cds-AF52_RS25155, NZ_KK737546.1, 622341, 622724, -, 384, 4803; cds-WP_013815099.1-16, NZ_KK737546.1, 622777, 623745, -, 969, 82668; cds-AF52_RS19995, NZ_KK737546.1, 623776, 625125, -, 1350, 10753; cds-WP _004179336.1, NZ_KK737546.1, 625161, 626168, -, 1008, 96294; cds-WP_004211823.1, NZ_KK737546.1, 626411, 627205, +, 795, 749; cds-WP _004140612.1, NZ_KK737546.1, 627241, 628203, +, 963, 103882; cds-WP_032429531.1, NZ_KK737546.1, 628228, 628572, -, 345, 40962; cds-WP _004179333.1, NZ_KK737546.1, 628663, 629493, -, 831, 134505; cds-WP_004140619.1, NZ_KK737546.1, 629508, 630419, -, 912, 179458; cds-WP _002900801.1, NZ_KK737546.1, 630468, 631712, -, 1245, 77452; cds-WP _002900798.1, NZ_KK737546.1, 631712, 632413, -, 702, 71743; cds-WP _004176566.1, NZ_KK737546.1, 632406, 633605, -, 1200, 182418; cds-WP _004140630.1, NZ_KK737546.1, 633739, 633870, -, 132, 8111; cds-WP _016946615.1, NZ_KK737546.1, 633910, 637356, +, 3447, 544299; cds-WP _023159039.1, NZ_KK737546.1, 637514, 638479, +, 966, 267974; cds-WP_002900788.1, NZ_KK737546.1, 638608, 638865, -, 258, 114310; cds-WP _004147941.1, NZ_KK737546.1, 638963, 639118, +, 156,9701; cds-WP _004140640.1, NZ_KK737546.1, 639115, 639750, +, 636, 98084; cds-WP _004140642.1, NZ_KK737546.1, 639803, 639925, -, 123, 22483; cds-WP _004150817.1, NZ_KK737546.1, 639994, 642183, +, 2190, 2772151; cds-WP_002900781.1, NZ_KK737546.1, 642241, 642780, -, 540, 111735; cds-WP_002900778.1, NZ_KK737546.1, 642975, 644279, -, 1305, 114871; cds-WP _002900775.1, NZ_KK737546.1, 644525, 645067, -, 543, 293387; cds-WP _004147935.1, NZ_KK737546.1, 645103, 646113, -, 1011, 298912; cds-WP_023286229.1, NZ_KK737546.1, 646137, 646964, -, 828, 54277; cds-WP _014342994.1, NZ_KK737546.1, 646945, 647583, -, 639, 145031; cds-WP_002900683.1, NZ_KK737546.1, 647605, 647979, -, 375, 119400; cds-WP_002900681.1, NZ_KK737546.1, 647984, 648340, -, 357, 63987; cds-WP_002900680.1, NZ_KK737546.1, 648602, 650035, -, 1434, 2067333; cds-WP _009484238.1, NZ_KK737546.1, 650332, 651126, -, 795, 133394; cds-WP _021441547.1, NZ_KK737546.1, 651137, 652141, -, 1005, 166485; cds-WP_002900674.1, NZ_KK737546.1, 652138, 652779, -, 642, 94396; cds-WP _002900671.1, NZ_KK737546.1, 652769, 653791, -, 1023, 272915; cds-WP _002900669.1, NZ_KK737546.1, 653793, 654602, -, 810, 75679; cds-WP_032428777.1, NZ_KK737546.1, 654728, 655969, -, 1242, 2528380; cds-WP _000103754.1, NZ_KK737546.1, 656059, 656295, -, 237, 4349737; cds-WP _002899294.1, NZ_KK737546.1, 656506, 657240, -, 735, 3787661; cds-WP _009308402.1, NZ_KK737546.1, 657253, 658182, -, 930, 837616; cds-WP_002899288.1, NZ_KK737546.1, 658198, 659151, -, 954, 801173; cds-WP_004140697.1, NZ_KK737546.1, 659205, 660365, -, 1161, 2780718; cds-WP _000290724.1, NZ_KK737546.1, 660506, 660679, -, 174, 7861409; cds-WP_002899276.1, NZ_KK737546.1, 660696, 661217, -, 522, 16103854; cds-WP _004183641.1, NZ_KK737546.1, 661359, 661943, +, 585, 117482; cds-WP _002899271.1, NZ_KK737546.1, 661981, 662934, -, 954, 139504; cds-AF52_RS19800, NZ_KK737546.1, 663634, 666601, +, 2968, 5110388; cds-WP _014342992.1, NZ_KK737546.1, 666598, 666849, +, 252, 27867; cds-WP_004218670.1, NZ_KK737546.1, 666888, 667916, -, 1029, 6498; cds-WP_004176581.1, NZ_KK737546.1, 667933, 669147, +, 1215, 0; cds-WP_002899104.1, NZ_KK737546.1, 669202, 670737, -, 1536, 536958; cds-WP _004140718.1, NZ_KK737546.1, 670849, 671760, -, 912, 493945; cds-WP _004179323.1, NZ_KK737546.1, 671776, 672429, -, 654, 589320; cds-WP_002899099.1, NZ_KK737546.1, 672439, 673023, -, 585, 386355; cds-WP _002899096.1, NZ_KK737546.1, 673253, 674461, +, 1209, 65303; cds-WP_004150824.1, NZ_KK737546.1, 674575, 675135, +, 561, 387448; cds-WP _032428773.1, NZ_KK737546.1, 675261, 676307, +, 1047, 492273; cds-WP_002898994.1, NZ_KK737546.1, 676380, 676631, +, 252, 123986; cds-WP _002898992.1, NZ_KK737546.1, 676934, 677188, +, 255, 356709; cds-WP _014907855.1, NZ_KK737546.1, 677314, 678432, +, 1119, 205047; cds-WP_002898988.1, NZ_KK737546.1, 678504, 678617, +, 114, 54268; cds-WP_032429533.1, NZ_KK737546.1, 678603, 679667, -, 1065, 267887; cds-WP_004140735.1, NZ_KK737546.1, 679873, 680793, +, 921, 242186; cds-WP _002898984.1, NZ_KK737546.1, 680965, 682191, +, 1227, 3783; cds-WP_002898981.1, NZ_KK737546.1, 682283, 682669, +, 387, 11264; cds-WP_002898978.1, NZ_KK737546.1, 682670, 682897, -, 228, 11996; cds-WP _002898967.1, NZ_KK737546.1, 682964, 685492, -, 2529, 2457436; cds-WP_004183630.1, NZ_KK737546.1, 685485, 687038, -, 1554, 2763996; cds-WP _021441537.1, NZ_KK737546.1, 687290, 688450, +, 1161,
161371; cds-WP_004179313.1, NZ_KK737546.1, 688485, 689078, -, 594, 44901; cds-WP_021441536.1, NZ_KK737546.1, 689145, 689672, -, 528, 8715; cds-WP _004191006.1, NZ_KK737546.1, 689822, 690304, -, 483, 16763; cds-WP_004140758.1, NZ_KK737546.1, 690408, 690962, -, 555, 96957; cds-WP_004140764.1, NZ_KK737546.1, 690985, 691722, -, 738, 120652; cds-WP _032429534.1, NZ_KK737546.1, 691811, 692749, -, 939, 67714; cds-WP _020804646.1, NZ_KK737546.1, 693463, 694368, -, 906, 7242; cds-WP_032428769.1, NZ_KK737546.1, 694483, 695343, +, 861, 4063; cds-WP_002898927.1, NZ_KK737546.1, 695362, 696039, +, 678, 65750; cds-WP _013815099.1-17, NZ_KK737546.1, 696673, 697641, +, 969, 85984; cds-AF52_RS0123255, NZ_KK737546.1, 697961, 698214, +, 254, 1095; cds-WP_148673676.1, NZ_KK737546.1, 698298, 699011, +, 714, 24790; cds-WP _072001576.1, NZ_KK737546.1, 699534, 699737, -, 204, 1027; cds-WP_020806188.1, NZ_KK737546.1, 699763, 700743, +, 981, 278320; cds-WP_002906471.1, NZ_KK737546.1, 701021, 701311, -, 291, 10; cds-AF52_RS0123260, NZ_KK737546.1, 701331, 702492, -, 1162, 3186; cds-WP_187079186.1, NZ_KK737546.1, 702608, 703498, +, 891, 67740; cds-WP _032429537.1, NZ_KK737546.1, 703493, 704992, -, 1500, 367746; cds-WP_021440209.1, NZ_KK737546.1, 705116, 705691, +, 576, 332162; cds-WP_004151231.1, NZ_KK737546.1, 705866, 707248, +, 1383, 7333; cds-WP_021440211.1, NZ_KK737546.1, 707363, 711103, +, 3741, 31767; cds-WP _004148517.1, NZ_KK737546.1, 711100, 712644, +, 1545, 317; cds-WP _002906547.1, NZ_KK737546.1, 712644, 713339, +, 696, 7982; cds-WP_004180107.1, NZ_KK737546.1, 713336, 714016, +, 681,17705; cds-WP_032429538.1, NZ_KK737546.1, 714449, 715294, -, 846, 112919; cds-WP_021440213.1, NZ_KK737546.1, 715371, 717026, -, 1656, 181; cds-WP _004189830.1, NZ_KK737546.1, 717084, 718052, -, 969, 313; cds-WP_004189827.1, NZ_KK737546.1, 718049, 718753, -, 705, 57; cds-WP_004151228.1, NZ_KK737546.1, 718965, 719546, +, 582, 32211; cds-WP _021440214.1, NZ_KK737546.1, 719670, 719897, -, 228, 84008; cds-WP _021440215.1, NZ_KK737546.1, 719968, 720585, -, 618, 34906; cds-WP _014343117.1, NZ_KK737546.1, 720663, 721514, -, 852, 15500; cds-WP_002906697.1, NZ_KK737546.1, 721553, 722332, -, 780, 0; cds-WP_021440216.1, NZ_KK737546.1, 722316, 723242, -, 927, 32780; cds-WP_021440217.1, NZ_KK737546.1, 723252, 723761, -, 510, 1145; cds-WP_032429539.1, NZ_KK737546.1, 723773, 724705, -, 933, 0; cds-WP _032429540.1, NZ_KK737546.1, 724723, 726036, -, 1314, 1625; cds-WP_032429541.1, NZ_KK737546.1, 726061, 727182, -, 1122, 4; cds-WP_004898544.1, NZ_KK737546.1, 727172, 728179, -, 1008, 4680; cds-WP _004180132.1, NZ_KK737546.1, 728271, 728822, -, 552, 2; cds-WP _021440220.1, NZ_KK737546.1, 729096, 729983, +, 888, 53738; cds-WP _002906778.1, NZ_KK737546.1, 730173, 731627, +, 1455, 681026; cds-WP_002906779.1, NZ_KK737546.1, 731678, 731899, -, 222, 36126; cds-WP_032429542.1, NZ_KK737546.1, 732068, 733129, -, 1062, 733351; cds-WP _032429543.1, NZ_KK737546.1, 733388, 735493, +, 2106, 251082; cds-AF52_RS0123265, NZ_KK737546.1, 735691, 737396, +, 1706, 39278; cds-WP_002906788.1, NZ_KK737546.1, 737494, 737793, -, 300, 8258; cds-WP_032428300.1, NZ_KK737546.1, 737883, 738113, -, 231, 123; cds-WP_021440225.1, NZ_KK737546.1, 738075, 739010, +, 936, 166272; cds-WP_002906792.1, NZ_KK737546.1, 739074, 740111, -, 1038, 262936; cds-WP _002906794.1, NZ_KK737546.1, 740177, 740758, -, 582, 106049; cds-WP _002906796.1, NZ_KK737546.1, 740921, 741439, +, 519, 56835; cds-WP _004180135.1, NZ_KK737546.1, 741436, 741885, +, 450, 97896; cds-WP _002906801.1, NZ_KK737546.1, 741894, 742127, -, 234, 50559; cds-WP _009484786.1, NZ_KK737546.1, 742144, 743742, -, 1599, 246499; cds-WP _021440226.1, NZ_KK737546.1, 744012, 745445, +, 1434, 83200; cds-WP _004200090.1, NZ_KK737546.1, 745459, 746208, -, 750, 51219; cds-AF52_RS25200, NZ_KK737546.1, 746297, 746392, -, 96, 14; cds-WP_013815099.1-18, NZ_KK737546.1, 746451, 747419, -, 969, 90897; cds-AF52_RS0123285, NZ_KK737546.1, 747453, 747536, -, 84, 0; cds-WP _002906891.1, NZ_KK737546.1, 747732, 747824, +, 93, 633; cds-AF52_RS0123290, NZ_KK737546.1, 747881, 748849, +, 969, 120621; cds-WP_002906893.1, NZ_KK737546.1, 748942, 749193, -, 252, 60062; cds-WP_004189776.1, NZ_KK737546.1, 749331, 749528, -, 198, 8701; cds-WP_004175940.1, NZ_KK737546.1, 749677, 749799, +, 123, 3704; cds-WP _004175939.1, NZ_KK737546.1, 750140, 751567, -, 1428, 11487; cds-WP _004143773.1, NZ_KK737546.1, 751590, 752396, -, 807, 182; cds-WP _032429544.1, NZ_KK737546.1, 752386, 753315, -, 930, 41; cds-WP_004151215.1, NZ_KK737546.1, 753317, 754330, -, 1014, 1013; cds-AF52_RS0123305, NZ_KK737546.1, 754347, 755492, -, 1146, 51978; cds-WP _032429724.1, NZ_KK737546.1, 755797, 757209, -, 1413, 262555; cds-WP _032429545.1, NZ_KK737546.1, 757897, 759294, +, 1398, 3629; cds-WP_020802114.1, NZ_KK737546.1, 759294, 760304, +, 1011, 3082; cds-WP_004180143.1, NZ_KK737546.1, 760304, 760429, +, 126, 0; cds-WP_187079190.1, NZ_KK737546.1, 760525, 760692, +, 168, 2900; cds-WP_013815099.1-19, NZ_KK737546.1, 760738, 761706, -, 969, 75405; cds-WP_077254734.1, NZ_KK737546.1, 761708, 761899, -, 192, 262; cds-WP _004143781.1, NZ_KK737546.1, 762045, 762275, +, 231, 6005; cds-WP_032429546.1, NZ_KK737546.1, 762288, 764249, -, 1962, 696215; cds-WP_020806151.1, NZ_KK737546.1, 764283, 764450, +, 168, 32855; cds-WP _002907473.1, NZ_KK737546.1, 764461, 765030, -, 570, 37906; cds-WP_032428296.1, NZ_KK737546.1, 765216, 766382, +, 1167, 948; cds-WP_004151213.1, NZ_KK737546.1, 766359, 767228, -, 870, 10800; cds-WP_004151212.1, NZ_KK737546.1, 767409, 768305, +, 897, 31741; cds-WP_002907480.1, NZ_KK737546.1, 768352, 769020, -, 669, 146312; cds-WP_021440235.1, NZ_KK737546.1, 769281, 769877, -, 597, 7403; cds-WP_002907483.1, NZ_KK737546.1, 769874, 770878, -, 1005, 12453; cds-WP_004891515.1, NZ_KK737546.1, 770990, 771970, +, 981, 43801; cds-WP _002907487.1, NZ_KK737546.1, 771956, 772534, -, 579, 162222; cds-WP _002907489.1, NZ_KK737546.1, 772588, 774243, -, 1656, 1298757; cds-WP _032428297.1, NZ_KK737546.1, 774681, 775769, +, 1089, 12845; cds-WP _021440237.1, NZ_KK737546.1, 775816, 777321, -, 1506, 18050; cds-WP _002907549.1, NZ_KK737546.1, 777363, 778706, -, 1344, 17867; cds-WP _021440238.1, NZ_KK737546.1, 778946, 779686, +, 741, 47166; cds-WP_004143799.1, NZ_KK737546.1, 779683, 780303, +, 621, 1015; cds-WP_004143800.1, NZ_KK737546.1, 780481, 781404, +, 924, 1601215; cds-WP _021440239.1, NZ_KK737546.1, 781704, 784076, +, 2373, 92031; cds-WP _004143804.1, NZ_KK737546.1, 784146, 785264, -, 1119, 249721; cds-WP_002907563.1, NZ_KK737546.1, 785289, 785564, -, 276, 60900; cds-WP _002907640.1, NZ_KK737546.1, 785669, 786196, -, 528, 123504; cds-WP_004143806.1, NZ_KK737546.1, 786456, 786818, +, 363, 69245; cds-WP_002907644.1, NZ_KK737546.1, 786828, 787013, -, 186, 80535; cds-WP_032429547.1, NZ_KK737546.1, 787257, 787721, -, 465, 86599; cds-WP_032428298.1, NZ_KK737546.1, 787915, 789069, +, 1155, 1278270; cds-WP_004151209.1, NZ_KK737546.1, 789227, 790228, +, 1002, 1341336; cds-WP_021440243.1, NZ_KK737546.1, 790264, 791304, -, 1041, 123889; cds-WP_004227851.1, NZ_KK737546.1, 791495, 791632, +, 138, 44745; cds-WP_021440244.1, NZ_KK737546.1, 791748, 793118, +, 1371, 146732; cds-WP _002907652.1, NZ_KK737546.1, 793335, 793550, +, 216, 270834; cds-WP_002907654.1, NZ_KK737546.1, 793626, 794066, +, 441, 121849; cds-WP_002907658.1, NZ_KK737546.1, 794145, 794726, +, 582, 527488; cds-WP_004143816.1, NZ_KK737546.1, 794726, 795304, +, 579, 211043; cds-AF52_RS19200, NZ_KK737546.1, 795297, 797532, +, 2236, 334540; cds-WP_002907730.1, NZ_KK737546.1, 797533, 798585, +, 1053, 412447; cds-WP _002907731.1, NZ_KK737546.1, 798596, 799216, +, 621, 209627; cds-WP _002907733.1, NZ_KK737546.1, 799219, 799917, +, 699, 149587; cds-WP_032429549.1, NZ_KK737546.1, 799919, 800554, +, 636, 333083; cds-WP_004151206.1, NZ_KK737546.1, 801162, 802667, +, 1506, 1880325; cds-WP _021440245.1, NZ_KK737546.1, 802778, 803383, +, 606, 109826; cds-WP_032428303.1, NZ_KK737546.1, 803426, 804286, -, 861, 103301; cds-WP_032429550.1, NZ_KK737546.1, 804348, 805622, -, 1275, 782225; cds-WP _032428304.1, NZ_KK737546.1, 805747, 806403, -, 657, 179793; cds-WP _032428306.1, NZ_KK737546.1, 806461, 806784, -, 324, 140521; cds-WP _004143831.1, NZ_KK737546.1, 806877, 808001, -, 1125, 154406; cds-WP_002907749.1, NZ_KK737546.1, 808269, 808736, +, 468, 133004; cds-AF52_RS19130, NZ_KK737546.1, 808959, 810134, +, 1176, 76525; cds-WP _013815099.1-20, NZ_KK737546.1, 810186, 811154, -, 969, 89311; cds-AF52_RS25210, NZ_KK737546.1, 811180, 811386, +, 207, 21469; cds-WP _004143833.1, NZ_KK737546.1, 811420, 811860, -, 441, 294271; cds-WP_002907752.1, NZ_KK737546.1, 812033, 812269, +, 237, 13001; cds-WP_004143835.1, NZ_KK737546.1, 812280, 813176, +, 897, 60162; cds-WP _002907754.1, NZ_KK737546.1, 813176, 815200, +, 2025, 42961; cds-WP_032429551.1, NZ_KK737546.1, 815193, 815711, -, 519, 43543; cds-WP_021440254.1, NZ_KK737546.1, 815781, 816677, -, 897, 774078; cds-WP _004189722.1, NZ_KK737546.1, 816726, 816968, -, 243, 4707; cds-WP _004175857.1, NZ_KK737546.1, 817108, 817707, +, 600, 30718; cds-WP _002907763.1, NZ_KK737546.1, 817761, 818858, +, 1098, 64695; cds-WP _013815099.1-21, NZ_KK737546.1, 818914, 819882, +, 969, 76709; cds-WP _002907764.1, NZ_KK737546.1, 820003, 820410, +, 408, 26548; cds-WP_002907766.1, NZ_KK737546.1, 820548, 821195, +, 648, 152129; cds-WP_032429552.1, NZ_KK737546.1, 821243, 821590, -, 348, 218025; cds-WP _004151204.1, NZ_KK737546.1, 821931, 822803, +, 873, 297257; cds-WP_004200127.1, NZ_KK737546.1, 822758, 823243, -, 486, 96695; cds-WP _002907771.1, NZ_KK737546.1, 823713, 824294, +, 582, 544830; cds-WP_002907773.1, NZ_KK737546.1, 824356, 825522, -, 1167, 204037; cds-WP_004152976.1, NZ_KK737546.1, 825690, 825779, -, 90, 19582; cds-WP_002907778.1, NZ_KK737546.1, 826075, 827100, +, 1026, 62844; cds-WP_004180170.1, NZ_KK737546.1, 827027, 828031, -, 1005, 25938; cds-WP _002907785.1, NZ_KK737546.1, 828144, 829325, +, 1182, 33014; cds-WP _032429553.1, NZ_KK737546.1, 829618, 830766, +, 1149, 342801; cds-WP _021440257.1, NZ_KK737546.1, 830803, 831438, -, 636, 157108; cds-WP_032429554.1, NZ_KK737546.1, 831668, 833041, +, 1374, 80544; cds-WP_004180172.1, NZ_KK737546.1, 833217, 833453, +, 237, 142; cds-AF52_RS0123325, NZ_KK737546.1, 833594, 834171, -, 578, 24175; cds-AF52_RS25220, NZ_KK737546.1, 835237, 835697, -, 461, 2239; cds-WP _032428308.1, NZ_KK737546.1, 836525, 837277, -, 753, 10; cds-WP _004180175.1, NZ_KK737546.1, 837376, 838302, +, 927, 4408; cds-WP_002907799.1, NZ_KK737546.1, 838299, 839183, -, 885, 4001; cds-WP_021440259.1, NZ_KK737546.1, 839340, 839855, +, 516, 0; cds-WP_004184289.1, NZ_KK737546.1, 839863, 840699, +, 837, 2175; cds-WP_002907802.1, NZ_KK737546.1, 840787, 840936, -, 150, 2077; cds-WP _004143863.1, NZ_KK737546.1, 841338, 841772, +, 435, 196; cds-WP_021440261.1, NZ_KK737546.1, 841818, 842534, +, 717, 28591; cds-WP_002907807.1, NZ_KK737546.1, 842619, 842828, +, 210, 15401; cds-WP _002907811.1, NZ_KK737546.1, 843141, 843776, -, 636, 349048; cds-WP _002907812.1, NZ_KK737546.1, 844026, 844655, +, 630, 229762; cds-WP_004189677.1, NZ_KK737546.1, 844660, 845430, -, 771, 2016; cds-WP_004175834.1, NZ_KK737546.1, 845522, 845905, +, 384, 6203; cds-WP_004175829.1, NZ_KK737546.1, 845937, 846620, +, 684, 195293; cds-WP_032428309.1, NZ_KK737546.1, 846965, 848668, +, 1704, 30793; cds-WP_004175825.1, NZ_KK737546.1, 848712, 849671, +, 960, 0; cds-WP _016197619.1, NZ_KK737546.1, 849668, 850618, +, 951, 11; cds-WP_021440263.1, NZ_KK737546.1, 850615, 851595, +, 981, 41; cds-WP _021440264.1, NZ_KK737546.1, 851592, 852563, +, 972, 0; cds-WP_002907918.1, NZ_KK737546.1, 852929, 853456, +, 528, 398887; cds-WP_032429555.1, NZ_KK737546.1, 853574, 854062, +, 489, 214127; cds-AF52_RS25225, NZ_KK737546.1, 854099, 854194, -, 96, 26421; cds-AF52_RS0123345, NZ_KK737546.1, 854208, 855739, -, 1532, 51773; cds-WP _021440267.1, NZ_KK737546.1, 855856, 856773, +, 918, 8233; cds-WP _021440268.1, NZ_KK737546.1, 856847, 857836, -, 990, 626; cds-WP_032429556.1, NZ_KK737546.1, 857857, 859275, -, 1419, 91; cds-WP _032428310.1, NZ_KK737546.1, 859287, 860276, -, 990, 0; cds-WP _021440271.1, NZ_KK737546.1, 860571, 861470, -, 900, 2548; cds-WP _032428312.1, NZ_KK737546.1, 861678, 862646, +, 969, 1211; cds-WP_021440273.1, NZ_KK737546.1, 862658, 863941, +, 1284, 6; cds-WP_002908128.1, NZ_KK737546.1, 863931, 864410, +, 480, 474; cds-WP_021440274.1, NZ_KK737546.1, 864407, 865288, +, 882, 11385; cds-WP_002908130.1, NZ_KK737546.1,
865288, 866271, +, 984, 27568; cds-WP_032429558.1, NZ_KK737546.1, 866268, 867098, +, 831, 0; cds-WP_002908134.1, NZ_KK737546.1, 867100, 867915, +, 816, 0; cds-WP_032429559.1, NZ_KK737546.1, 867942, 869363, +, 1422, 260; cds-WP_026005880.1, NZ_KK737546.1, 869443, 869691, +, 249, 2595; cds-WP_004145368.1, NZ_KK737546.1, 869857, 870765, -, 909, 1146594; cds-WP_002908189.1, NZ_KK737546.1, 871055, 871195, +, 141, 11816; cds-WP_004189584.1, NZ_KK737546.1, 871242, 872132, -, 891, 16076; cds-WP_004175762.1, NZ_KK737546.1, 872273, 872980, +, 708, 8776; cds-WP_021440277.1, NZ_KK737546.1, 872982, 873638, +, 657, 20433; cds-WP_032429560.1, NZ_KK737546.1, 873648, 874829, +, 1182,0; cds-WP_004891326.1, NZ_KK737546.1, 874841, 875764, +, 924, 78; cds-WP_032428313.1, NZ_KK737546.1, 875812, 877203, +, 1392, 2622; cds-WP_002908202.1, NZ_KK737546.1, 877227, 877997, +, 771, 9791; cds-WP_004145376.1, NZ_KK737546.1, 878017, 878889, -, 873, 10098; cds-WP_002908205.1, NZ_KK737546.1, 878996, 879775, +, 780, 46; cds-WP_004200174.1, NZ_KK737546.1, 879785, 881464, +, 1680, 352; cds-WP_004151179.1, NZ_KK737546.1, 881488, 882258, +, 771, 4382; cds-WP_013815099.1-22, NZ_KK737546.1, 883311, 884279, -, 969, 92098; cds-AF52_RS0123360, NZ_KK737546.1, 884312, 884632, +, 321, 33876; cds-WP_032428316.1, NZ_KK737546.1, 885134, 885976, -, 843, 14; cds-WP_014599186.1, NZ_KK737546.1, 885973, 886419, -, 447, 955; cds-WP_002908216.1, NZ_KK737546.1, 886431, 886985, -, 555, 78; cds-WP_021440283.1, NZ_KK737546.1, 886982, 888934, -, 1953, 51; cds-WP_021440284.1, NZ_KK737546.1, 888931, 889389, -, 459, 9698; cds-WP_021440285.1, NZ_KK737546.1, 889373, 889603, -, 231, 0; cds-WP_004189553.1, NZ_KK737546.1, 889600, 890337, -, 738, 57; cds-WP_032428317.1, NZ_KK737546.1, 890387, 891046, -, 660, 240; cds-WP_004151165.1, NZ_KK737546.1, 891043, 891666, -, 624, 1700; cds-WP_182920176.1, NZ_KK737546.1, 891750, 893090, -, 1341, 14620; cds-WP_002908285.1, NZ_KK737546.1, 893101, 894885, -, 1785, 28068; cds-WP_004217200.1, NZ_KK737546.1, 894888, 895643, -, 756, 49; cds-WP_002908289.1, NZ_KK737546.1, 896145, 896516, +, 372, 2396; cds-WP_002908292.1, NZ_KK737546.1, 896524, 897594, -, 1071, 21042; cds-WP_002908294.1, NZ_KK737546.1, 897587, 899356, -, 1770, 318384; cds-WP_002908297.1, NZ_KK737546.1, 899430, 900518, -, 1089, 423628; cds-WP_021313976.1, NZ_KK737546.1, 900821, 901900, +, 1080, 11349; cds-WP_021440289.1, NZ_KK737546.1, 902147, 904297, +, 2151, 3220; cds-WP_014343153.1, NZ_KK737546.1, 904340, 904501, -, 162, 0; cds-AF52_RS18635, NZ_KK737546.1, 904465, 905497, -, 1033, 24885; cds-WP_004148650.1, NZ_KK737546.1, 905793, 906992, +, 1200, 4290; cds-WP_004175720.1, NZ_KK737546.1, 907108, 907542, +, 435, 29713; cds-WP_032428318.1, NZ_KK737546.1, 908108, 909340, +, 1233, 21231; cds-WP_002908370.1, NZ_KK737546.1, 909377, 909769, +, 393, 46198; cds-WP_021440293.1, NZ_KK737546.1, 909898, 910485, -, 588, 0; cds-WP_002908374.1, NZ_KK737546.1, 910785, 911507, -, 723, 28975; cds-WP_032429562.1, NZ_KK737546.1, 911494, 912258, -, 765, 12076; cds-WP_032428319.1, NZ_KK737546.1, 912322, 913089, -, 768, 46487; cds-WP_002908378.1, NZ_KK737546.1, 913182, 914084, -, 903, 7792; cds-WP_004148675.1, NZ_KK737546.1, 914081, 915058, -, 978, 21043; cds-WP_021440296.1, NZ_KK737546.1, 915048, 916100, -, 1053,622; cds-WP_004213478.1, NZ_KK737546.1, 916097, 917122, -, 1026, 33791; cds-WP_002908416.1, NZ_KK737546.1, 917119, 917940, -, 822, 50221; cds-WP_004891261.1, NZ_KK737546.1, 918446, 920518, +, 2073, 22304; cds-WP_004175711.1, NZ_KK737546.1, 920627, 921400, -, 774, 0; cds-WP_004148681.1, NZ_KK737546.1, 921394, 922389, -, 996, 0; cds-WP_032429563.1, NZ_KK737546.1, 922370, 923407, -, 1038, 3654; cds-WP_002908454.1, NZ_KK737546.1, 923404, 924366, -, 963, 10; cds-WP_032429564.1, NZ_KK737546.1, 924372, 925523, -, 1152, 94; cds-WP_016947511.1, NZ_KK737546.1, 925520, 926605, -, 1086, 45; cds-WP_032429565.1, NZ_KK737546.1, 926750, 927643, -, 894, 163; cds-WP_002908526.1, NZ_KK737546.1, 927750, 928352, +, 603, 12; cds-WP_021440299.1, NZ_KK737546.1, 928358, 929938, +, 1581, 4421; cds-WP_021440300.1, NZ_KK737546.1, 929951, 930676, -, 726, 21145; cds-AF52_RS0123375, NZ_KK737546.1, 930779, 931975, -, 1197, 1250; cds-WP_004180340.1, NZ_KK737546.1, 932051, 933067, -, 1017, 348; cds-WP_021440302.1, NZ_KK737546.1, 933064, 934014, -, 951, 3534; cds-WP_032429566.1, NZ_KK737546.1, 934007, 934813, -, 807, 0; cds-WP_004175683.1, NZ_KK737546.1, 934824, 935690, -, 867, 138; cds-WP_021440303.1, NZ_KK737546.1, 935708, 936652, -, 945, 99; cds-WP_024622675.1, NZ_KK737546.1, 936654, 938318, -, 1665, 39230; cds-WP_004184404.1, NZ_KK737546.1, 938496, 939308, +, 813, 2789; cds-WP_016530930.1, NZ_KK737546.1, 939400, 940458, +, 1059, 80742; cds-WP_004151840.1, NZ_KK737546.1, 940448, 941455, +, 1008, 2; cds-WP_004151841.1, NZ_KK737546.1, 941452, 942201, +, 750, 502; cds-WP_014907413.1, NZ_KK737546.1, 942185, 942928, -, 744, 12657; cds-WP_004175664.1, NZ_KK737546.1, 942985, 943821, -, 837, 53670; cds-WP_004148701.1, NZ_KK737546.1, 943837, 945159, -, 1323, 45470; cds-WP_032429567.1, NZ_KK737546.1, 945231, 947084, -, 1854, 29961; cds-WP_002908844.1, NZ_KK737546.1, 947211, 947849, +, 639, 40032; cds-WP_004180357.1, NZ_KK737546.1, 947867, 948079, -, 213, 20153; cds-WP_014907411.1, NZ_KK737546.1, 948069, 948227, -, 159, 2178; cds-WP_004217210.1, NZ_KK737546.1, 948316, 948447, -, 132, 27626; cds-WP_002908859.1, NZ_KK737546.1, 948585, 949997, +, 1413, 1803984; cds-WP_032429569.1, NZ_KK737546.1, 950310, 950546, +, 237, 3280507; cds-WP_004143241.1, NZ_KK737546.1, 950608, 951594, -, 987, 268703; cds-WP_002908865.1, NZ_KK737546.1, 951694, 952110, -, 417, 85331; cds-WP_002908867.1, NZ_KK737546.1, 952122, 953342, -, 1221, 39769; cds-AF52_RS18405, NZ_KK737546.1, 953339, 954046, -, 708, 32; cds-WP_013815099.1-23, NZ_KK737546.1, 954079, 955047, +, 969, 119323; cds-AF52_RS18395, NZ_KK737546.1, 955104, 955679, -, 576, 35363; cds-WP_002908876.1, NZ_KK737546.1, 955654, 956400, -, 747, 114860; cds-WP_002908877.1, NZ_KK737546.1, 956417, 957904, -, 1488, 310645; cds-WP_004143232.1, NZ_KK737546.1, 957907, 958284, -, 378, 21374; cds-WP_002908880.1, NZ_KK737546.1, 958767, 958964, -, 198, 1915; cds-WP_014343173.1, NZ_KK737546.1, 958963, 959100, +, 138, 342; cds-WP_004143228.1, NZ_KK737546.1, 959201, 959596, +, 396, 3546; cds-WP_002908882.1, NZ_KK737546.1, 959593, 960225, +, 633, 21772; cds-WP_004151846.1, NZ_KK737546.1, 960480, 961664, +, 1185, 2465; cds-WP_021440309.1, NZ_KK737546.1, 961720, 964812, -, 3093, 20562; cds-WP_032429570.1, NZ_KK737546.1, 964809, 965918, -, 1110, 3403; cds-WP_020805192.1, NZ_KK737546.1, 965993, 966529, -, 537, 19513; cds-WP_021440311.1, NZ_KK737546.1, 966703, 967698, -, 996, 52150; cds-WP_004175653.1, NZ_KK737546.1, 967795, 968706, +, 912, 9886; cds-WP_004180366.1, NZ_KK737546.1, 968865, 969863, +, 999, 163694; cds-WP_002908998.1, NZ_KK737546.1, 969863, 970900, +, 1038, 86793; cds-WP_004212506.1, NZ_KK737546.1, 970900, 971661, +, 762, 209796; cds-WP_182920187.1, NZ_KK737546.1, 971718, 972905, +, 1188, 187314; cds-WP_002909005.1, NZ_KK737546.1, 972988, 974019, -, 1032, 48590; cds-WP_002909008.1, NZ_KK737546.1, 974043, 975023, -, 981, 40005; cds-WP_004212509.1, NZ_KK737546.1, 975593, 975913, +, 321, 148895; cds-WP_002909014.1, NZ_KK737546.1, 976011, 976259, -, 249, 59621; cds-WP_002909015.1, NZ_KK737546.1, 976464, 976874, -, 411, 42030; cds-WP_004175614.1, NZ_KK737546.1, 976871, 979927, -, 3057, 246657; cds-WP_004148729.1, NZ_KK737546.1, 980146, 981258, +, 1113, 76160; cds-WP_002909055.1, NZ_KK737546.1, 981641, 984019, -, 2379, 3907518; cds-WP_009484225.1, NZ_KK737546.1, 984095, 984214, +, 120, 935; cds-WP_002909061.1, NZ_KK737546.1, 984359, 985192, +, 834, 318327; cds-WP_021440317.1, NZ_KK737546.1, 985347, 986393, +, 1047, 122406; cds-WP_002909070.1, NZ_KK737546.1, 986501, 986728, +, 228, 24091; cds-WP_021440318.1, NZ_KK737546.1, 986758, 988200, -, 1443, 372190; cds-WP_002909082.1, NZ_KK737546.1, 988314, 988778, -, 465, 39613; cds-WP_020803964.1, NZ_KK737546.1, 988860, 989609, -, 750, 115809; cds-WP_004180371.1, NZ_KK737546.1, 989609, 990160, -, 552, 21685; cds-WP_002909085.1, NZ_KK737546.1, 990397, 991197, +, 801, 393433; cds-WP_004151850.1, NZ_KK737546.1, 991234, 992220, -, 987, 62671; cds-WP_014907393.1, NZ_KK737546.1, 992342, 993775, -, 1434, 44410; cds-WP_002909089.1, NZ_KK737546.1, 994056, 994970, +, 915, 128855; cds-WP_020802655.1, NZ_KK737546.1, 995020, 996273, +, 1254, 83757; cds-WP_021440321.1, NZ_KK737546.1, 996270, 996623, +, 354, 3002; cds-WP_002909098.1, NZ_KK737546.1, 996676, 996975, -, 300, 651882; cds-WP_015958605.1, NZ_KK737546.1, 996980, 999367, -, 2388, 2685901; cds-WP_002909105.1, NZ_KK737546.1, 999383, 1000366, -, 984, 790367; cds-WP_001386830.1, NZ_KK737546.1, 1000505, 1000549, -, 45, 984; cds-WP_000124850.1, NZ_KK737546.1, 1000673, 1001029, -, 357, 11167969; cds-WP_001124225.1, NZ_KK737546.1, 1001080, 1001277, -, 198, 7290760; cds-WP_004189469.1, NZ_KK737546.1, 1001370, 1001912, -, 543, 8581305; cds-WP_032429575.1, NZ_KK737546.1, 1001916, 1003844, -, 1929, 8369298; cds-WP_002910028.1, NZ_KK737546.1, 1004276, 1004509, -, 234, 3434; cds-WP_002910030.1, NZ_KK737546.1, 1004781, 1005539, -, 759, 139199; cds-WP_032429576.1, NZ_KK737546.1, 1005896, 1006828, +, 933, 25256; cds-WP_004184572.1, NZ_KK737546.1, 1006923, 1007207, +, 285, 25485; cds-WP_032429577.1, NZ_KK737546.1, 1007313, 1008185, +, 873, 302380; cds-WP_014343181.1, NZ_KK737546.1, 1008229, 1009017, -, 789, 206279; cds-WP_002910042.1, NZ_KK737546.1, 1009186, 1009482, +, 297, 129044; cds-WP_032103891.1, NZ_KK737546.1, 1009595, 1009735, +, 141, 2526; cds-WP_072271743.1, NZ_KK737546.1, 1010163, 1010810, +, 648, 13558; cds-WP_004194006.1, NZ_KK737546.1, 1011313, 1011486, -, 174, 1343; cds-WP_002910077.1, NZ_KK737546.1, 1011630, 1013090, +, 1461, 510612; cds-WP_002910079.1, NZ_KK737546.1, 1013125, 1013454, +, 330, 37066; cds-WP_002910080.1, NZ_KK737546.1, 1013517, 1014344, -, 828, 214381; cds-WP_002910083.1, NZ_KK737546.1, 1014400, 1015404, -, 1005, 282745; cds-WP_004151853.1, NZ_KK737546.1, 1015401, 1016414, -, 1014, 412449; cds-WP_004145396.1, NZ_KK737546.1, 1016426, 1017334, -, 909, 310605; cds-WP_002910089.1, NZ_KK737546.1, 1017349, 1018269, -, 921, 144698; cds-WP_004148754.1, NZ_KK737546.1, 1018355, 1019989, -, 1635, 2245889; cds-WP_002910095.1, NZ_KK737546.1, 1020727, 1021374, -, 648, 25320; cds-WP_004145398.1, NZ_KK737546.1, 1021558, 1021773, -, 216, 38; cds-WP_002910097.1, NZ_KK737546.1, 1021851, 1024526, +, 2676, 2669653; cds-WP_002910100.1, NZ_KK737546.1, 1024674, 1025291, -, 618, 75555; cds-WP_004145401.1, NZ_KK737546.1, 1025299, 1025430, +, 132, 1594; cds-WP_002910103.1, NZ_KK737546.1, 1025853, 1026260, +, 408, 2182248; cds-WP_002910105.1, NZ_KK737546.1, 1026382, 1027284, -, 903, 3193508; cds-WP_002910107.1, NZ_KK737546.1, 1027482, 1028495, -, 1014, 224160; cds-WP_002910108.1, NZ_KK737546.1, 1028585, 1029487, -, 903, 413028; cds-WP_032429578.1, NZ_KK737546.1, 1029600, 1030058, +, 459, 338226; cds-WP_004151854.1, NZ_KK737546.1, 1030101, 1030943, +, 843, 474339; cds-WP_032428322.1, NZ_KK737546.1, 1031951, 1032394, -, 444, 180616; cds-WP_021440328.1, NZ_KK737546.1, 1032502, 1033398, +, 897, 317; cds-WP_032429579.1, NZ_KK737546.1, 1033395, 1034072, -, 678, 93625; cds-WP_002910191.1, NZ_KK737546.1, 1034072, 1034782, -, 711, 111746; cds-WP_019705142.1, NZ_KK737546.1, 1034779, 1036314, -, 1536, 99385; cds-WP_004180382.1, NZ_KK737546.1, 1036311, 1040054, -, 3744, 211732; cds-WP_032428329.1, NZ_KK737546.1, 1040221, 1040334, -, 114, 0; cds-WP_032428323.1, NZ_KK737546.1, 1040460, 1041848, -, 1389, 16754; cds-WP_004175566.1, NZ_KK737546.1, 1042160, 1043938, +, 1779, 78910; cds-WP_002910198.1, NZ_KK737546.1, 1043949, 1044599, +, 651, 72496; cds-WP_002910201.1, NZ_KK737546.1, 1044596, 1045978, -, 1383, 67689; cds-WP_004175564.1, NZ_KK737546.1, 1046130, 1048730, -, 2601, 3428; cds-WP_021440334.1, NZ_KK737546.1, 1048730, 1052797, -, 4068, 160; cds-WP_004145418.1, NZ_KK737546.1, 1052808, 1053596, -, 789, 0; cds-WP_004152255.1, NZ_KK737546.1, 1053606, 1054490, -, 885, 0; cds-WP_032429580.1, NZ_KK737546.1, 1054492, 1055748, -, 1257, 0; cds-WP_021440335.1, NZ_KK737546.1, 1055991, 1057172, -, 1182, 0; cds-WP_002910380.1, NZ_KK737546.1, 1057240, 1057593, +, 354, 96068; cds-WP_002910387.1, NZ_KK737546.1, 1057831, 1058073, +, 243, 41540; cds-WP_004152258.1, NZ_KK737546.1, 1058291, 1058971, -, 681, 102568; cds-WP_002910389.1,
NZ_KK737546.1, 1059108, 1059338, -, 231, 14910; cds-WP_002910392.1, NZ_KK737546.1, 1059602, 1060702, +, 1101, 395416; cds-WP_002910393.1, NZ_KK737546.1, 1060789, 1061643, -, 855, 1190831; cds-WP_032429581.1, NZ_KK737546.1, 1061683, 1062495, -, 813, 402633; cds-WP_004145428.1, NZ_KK737546.1, 1062499, 1062891, -, 393, 57633; cds-WP_004898979.1, NZ_KK737546.1, 1062891, 1063739, -, 849, 268295; cds-WP_002910403.1, NZ_KK737546.1, 1063739, 1064821, -, 1083, 118008; cds-WP_002910404.1, NZ_KK737546.1, 1064864, 1066120, -, 1257, 267787; cds-WP_002910405.1, NZ_KK737546.1, 1066391, 1067002, +, 612, 435167; cds-WP_004898981.1, NZ_KK737546.1, 1067002, 1067850, +, 849, 1069941; cds-WP_002910407.1, NZ_KK737546.1, 1068034, 1068981, +, 948, 5984409; cds-WP_004200291.1, NZ_KK737546.1, 1069106, 1070785, +, 1680, 195660; cds-WP_004175547.1, NZ_KK737546.1, 1070786, 1071832, -, 1047, 117709; cds-WP_002910437.1, NZ_KK737546.1, 1072054, 1072329, -, 276, 236728; cds-WP_004180389.1, NZ_KK737546.1, 1072602, 1073186, +, 585, 550048; cds-WP_002910443.1, NZ_KK737546.1, 1073304, 1074395, +, 1092, 1696062; cds-WP_004145442.1, NZ_KK737546.1, 1074477, 1074806, -, 330, 117884; cds-WP_002910453.1, NZ_KK737546.1, 1074890, 1075804, -, 915, 126143; cds-WP_032429582.1, NZ_KK737546.1, 1075936, 1077351, -, 1416, 0; cds-WP_004175538.1, NZ_KK737546.1, 1077371, 1077814, -, 444, 295; cds-WP_004175532.1, NZ_KK737546.1, 1077817, 1078353, -, 537, 0; cds-WP_021440342.1, NZ_KK737546.1, 1078334, 1079350, -, 1017, 83; cds-AF52_RS17835, NZ_KK737546.1, 1079380, 1081143, -, 1764, 219; cds-WP_171991785.1, NZ_KK737546.1, 1081277, 1084693, -, 3417, 55425; cds-WP_004899005.1, NZ_KK737546.1, 1084708, 1085919, -, 1212, 48158; cds-WP_004899008.1, NZ_KK737546.1, 1085924, 1086181, -, 258, 1207; cds-WP_013815099.1-24, NZ_KK737546.1, 1086223, 1087191, +, 969, 75293; cds-WP_071603213.1, NZ_KK737546.1, 1087273, 1088052, -, 780, 0; cds-AF52_RS25250, NZ_KK737546.1, 1088184, 1089413, -, 1230, 6210; cds-AF52_RS26885, NZ_KK737546.1, 1089482, 1089616, +, 135, 6244; cds-WP_187079183.1, NZ_KK737546.1, 1089947, 1090528, -, 582, 8381; cds-AF52_RS0123460, NZ_KK737546.1, 1090628, 1090912, -, 285, 37502; cds-AF52_RS26890, NZ_KK737546.1, 1095758, 1095891, -, 134, 10854; cds-AF52_RS25260, NZ_KK737546.1, 1096147, 1096341, +, 195, 10832; cds-WP_050484277.1, NZ_KK737546.1, 1096700, 1097497, -, 798, 1645; cds-AF52_RS26910, NZ_KK737546.1, 1101418, 1101547, -, 130, 3625; cds-WP_024622687.1, NZ_KK737546.1, 1101978, 1103318, +, 1341, 0; cds-WP_032429587.1, NZ_KK737546.1, 1103315, 1103968, +, 654, 0; cds-WP_032429588.1, NZ_KK737546.1, 1103972, 1105669, +, 1698, 0; cds-AF52_RS27855, NZ_KK737546.1, 1105856, 1105980, +, 125, 1266; cds-AF52_RS17780, NZ_KK737546.1, 1106131, 1108194, +, 2064, 14640; cds-WP_013815099.1-25, NZ_KK737546.1, 1108246, 1109214, -, 969, 71088; cds-WP_050484282.1, NZ_KK737546.1, 1109194, 1110111, +, 918, 74270; cds-WP_032429589.1, NZ_KK737546.1, 1110157, 1110477, -, 321, 9972; cds-WP_032429590.1, NZ_KK737546.1, 1110545, 1110808, -, 264, 660; cds-WP_032429591.1, NZ_KK737546.1, 1110819, 1111511, -, 693, 407; cds-WP_032429592.1, NZ_KK737546.1, 1111564, 1113084, -, 1521, 11383; cds-WP_032429593.1, NZ_KK737546.1, 1113085, 1114761, -, 1677, 53251; cds-WP_032429594.1, NZ_KK737546.1, 1114766, 1115335, -, 570, 7498; cds-WP_032429596.1, NZ_KK737546.1, 1115340, 1115555, -, 216, 2711; cds-WP_087749278.1, NZ_KK737546.1, 1115890, 1116027, -, 138, 4; cds-WP_032429598.1, NZ_KK737546.1, 1116030, 1116497, -, 468, 13020; cds-WP_020804048.1, NZ_KK737546.1, 1116494, 1117024, -, 531, 10697; cds-WP_004151282.1, NZ_KK737546.1, 1117027, 1117275, -, 249, 171; cds-WP_032429600.1, NZ_KK737546.1, 1118641, 1118994, -, 354, 545426; cds-WP_004884209.1, NZ_KK737546.1, 1119587, 1120087, -, 501, 32949; cds-WP_032429601.1, NZ_KK737546.1, 1120084, 1120224, -, 141, 12; cds-WP_032429603.1, NZ_KK737546.1, 1120221, 1120583, -, 363, 483; cds-WP_004141687.1, NZ_KK737546.1, 1120580, 1120870, -, 291, 1400; cds-WP_032429604.1, NZ_KK737546.1, 1120863, 1121033, -, 171, 1809; cds-WP_032429606.1, NZ_KK737546.1, 1121014, 1121481, -, 468, 3; cds-WP_032429607.1, NZ_KK737546.1, 1121740, 1121937, -, 198, 0; cds-WP_032429608.1, NZ_KK737546.1, 1121930, 1122235, -, 306, 173; cds-WP_077254738.1, NZ_KK737546.1, 1122226, 1122615, -, 390, 65040; cds-AF52_RS25290, NZ_KK737546.1, 1122697, 1122954, -, 258, 37718; cds-WP_001096658.1, NZ_KK737546.1, 1123182, 1123418, -, 237, 3; cds-WP_032429609.1, NZ_KK737546.1, 1123418, 1123792, -, 375, 2330; cds-WP_050484278.1, NZ_KK737546.1, 1123792, 1124439, -, 648, 99512; cds-WP_032429610.1, NZ_KK737546.1, 1124546, 1124806, -, 261, 52978; cds-WP_032429612.1, NZ_KK737546.1, 1124803, 1125198, -, 396, 12917; cds-AF52_RS0123495, NZ_KK737546.1, 1125195, 1125496, -, 302, 12; cds-WP_187079187.1, NZ_KK737546.1, 1125496, 1126911, -, 1416, 35789; cds-WP_187079188.1, NZ_KK737546.1, 1126916, 1127767, -, 852, 37543; cds-WP_004151298.1-2, NZ_KK737546.1, 1127808, 1127954, -, 147, 661; cds-WP_004141720.1-2, NZ_KK737546.1, 1128041, 1128361, -, 321, 6614; cds-WP_023284762.1, NZ_KK737546.1, 1128411, 1128626, -, 216, 3393; cds-WP_019704102.1, NZ_KK737546.1, 1128726, 1129358, +, 633, 159168; cds-WP_074440723.1, NZ_KK737546.1, 1129864, 1129989, +, 126, 58333; cds-WP_072001571.1, NZ_KK737546.1, 1129982, 1130188, +, 207, 56891; cds-WP_032429616.1, NZ_KK737546.1, 1130270, 1130554, +, 285, 95492; cds-AF52_RS27875, NZ_KK737546.1, 1130566, 1130764, +, 199, 8822; cds-WP_014342891.1, NZ_KK737546.1, 1130734, 1131069, +, 336, 7226; cds-WP_032429617.1, NZ_KK737546.1, 1131066, 1131689, +, 624, 37346; cds-WP_032429618.1, NZ_KK737546.1, 1131686, 1132114, +, 429, 3574; cds-WP_032429619.1, NZ_KK737546.1, 1132111, 1132767, +, 657, 37703; cds-WP_032429620.1, NZ_KK737546.1, 1132764, 1133933, +, 1170, 64092; cds-WP_032429621.1, NZ_KK737546.1, 1134147, 1134491, +, 345, 7496; cds-WP_072001572.1, NZ_KK737546.1, 1134484, 1134678, +, 195, 1; cds-WP_032429622.1, NZ_KK737546.1, 1134675, 1134866, +, 192, 485; cds-WP_032429623.1, NZ_KK737546.1, 1134863, 1135081, +, 219, 2; cds-WP_019725112.1, NZ_KK737546.1, 1135085, 1135327, +, 243, 14; cds-WP_032429624.1, NZ_KK737546.1, 1135375, 1136637, +, 1263, 107160; cds-AF52_RS0123510, NZ_KK737546.1, 1136676, 1136903, +, 228, 3756; cds-WP_004145468.1, NZ_KK737546.1, 1136878, 1137687, -, 810, 12884; cds-WP_004175494.1, NZ_KK737546.1, 1137699, 1138994, -, 1296, 1415; cds-WP_032429625.1, NZ_KK737546.1, 1139298, 1140224, -, 927, 121506; cds-WP_002910717.1, NZ_KK737546.1, 1140323, 1140799, +, 477, 137394; cds-WP_004148802.1, NZ_KK737546.1, 1140849, 1142492, -, 1644, 5085370; cds-WP_002910720.1, NZ_KK737546.1, 1142776, 1143669, +, 894, 335627; cds-WP_004175491.1, NZ_KK737546.1, 1143675, 1144394, -, 720, 22; cds-WP_004148803.1, NZ_KK737546.1, 1144391, 1145266, -, 876, 0; cds-WP_004148804.1, NZ_KK737546.1, 1145263, 1146549, -, 1287, 179; cds-WP_004175489.1, NZ_KK737546.1, 1146559, 1147473, -, 915, 378; cds-WP_004189329.1, NZ_KK737546.1, 1147583, 1148641, -, 1059, 0; cds-WP_004225356.1, NZ_KK737546.1, 1148931, 1149077, +, 147, 11; cds-WP_024622740.1, NZ_KK737546.1, 1149129, 1151423, -, 2295, 2733; cds-WP_021440352.1, NZ_KK737546.1, 1151452, 1152660, -, 1209, 13080; cds-WP_004145486.1, NZ_KK737546.1, 1152688, 1154019, -, 1332, 53606; cds-WP_004211070.1, NZ_KK737546.1, 1154045, 1155034, -, 990, 16; cds-WP_002910764.1, NZ_KK737546.1, 1155128, 1156069, -, 942, 57; cds-WP_004148810.1, NZ_KK737546.1, 1156246, 1156389, +, 144, 0; cds-WP_004145488.1, NZ_KK737546.1, 1156687, 1156809, +, 123, 113; cds-WP_024622741.1, NZ_KK737546.1, 1156847, 1157647, -, 801, 175; cds-WP_032425076.1, NZ_KK737546.1, 1157640, 1158626, -, 987, 682; cds-WP_002910830.1, NZ_KK737546.1, 1159065, 1159811, -, 747, 14229; cds-WP_008804319.1, NZ_KK737546.1, 1159828, 1160643, -, 816, 397; cds-WP_004184646.1, NZ_KK737546.1, 1160640, 1161569, -, 930, 238; cds-WP_004211064.1, NZ_KK737546.1, 1161563, 1163002, -, 1440, 882; cds-AF52_RS26335, NZ_KK737546.1, 1163005, 1163203, -, 199, 0; cds-WP_004175476.1, NZ_KK737546.1, 1163388, 1165133, +, 1746, 99609; cds-WP_023328505.1, NZ_KK737546.1, 1165384, 1167504, +, 2121, 364051; cds-WP_002910846.1, NZ_KK737546.1, 1167546, 1168157, -, 612, 386548; cds-WP_002910883.1, NZ_KK737546.1, 1168333, 1169247, +, 915, 1280; cds-WP_002910885.1, NZ_KK737546.1, 1169340, 1171073, +, 1734, 261506; cds-WP_002910887.1, NZ_KK737546.1, 1171090, 1172160, -, 1071, 1023236; cds-WP_004175465.1, NZ_KK737546.1, 1172170, 1173468, -, 1299, 2108262; cds-WP_032409324.1, NZ_KK737546.1, 1173567, 1173713, -, 147, 358; cds-WP_032429737.1, NZ_KK737546.1, 1173790, 1175322, +, 1533, 384016; cds-WP_002910890.1, NZ_KK737546.1, 1175397, 1176116, -, 720, 451966; cds-WP_004151442.1, NZ_KK737546.1, 1176365, 1177915, +, 1551, 290109; cds-WP_002910894.1, NZ_KK737546.1, 1178044, 1178574, +, 531, 167067; cds-WP_002910895.1, NZ_KK737546.1, 1178725, 1179069, +, 345, 22372; cds-WP_021440363.1, NZ_KK737546.1, 1179076, 1179522, -, 447, 148142; cds-WP_004180422.1, NZ_KK737546.1, 1179617, 1180276, -, 660, 231313; cds-WP_002910898.1, NZ_KK737546.1, 1180293, 1180574, -, 282, 56408; cds-WP_004180423.1, NZ_KK737546.1, 1180701, 1181399, +, 699, 617086; cds-WP_002910900.1, NZ_KK737546.1, 1181423, 1182235, +, 813, 2944661; cds-WP_002910902.1, NZ_KK737546.1, 1182239, 1182508, +, 270, 761645; cds-WP_187079189.1, NZ_KK737546.1, 1182586, 1183701, -, 1116, 144380; cds-WP_002910904.1, NZ_KK737546.1, 1183783, 1185468, -, 1686, 1632526; cds-WP_002910905.1, NZ_KK737546.1, 1185674, 1186255, -, 582, 60173; cds-WP_004151443.1, NZ_KK737546.1, 1186294, 1186989, -, 696, 61651; cds-WP_004175456.1, NZ_KK737546.1, 1187135, 1189045, -, 1911, 338260; cds-WP_002910910.1, NZ_KK737546.1, 1189176, 1189520, +, 345, 73588; cds-WP_004145519.1, NZ_KK737546.1, 1189521, 1189706, -, 186, 43011; cds-WP_004145520.1, NZ_KK737546.1, 1189795, 1191150, +, 1356, 139475; cds-WP_004180429.1, NZ_KK737546.1, 1191154, 1191732, +, 579, 74475; cds-WP_004151445.1, NZ_KK737546.1, 1191895, 1193259, +, 1365, 1416980; cds-WP_024622747.1, NZ_KK737546.1, 1193442, 1195025, +, 1584, 280834; cds-WP_004145524.1, NZ_KK737546.1, 1195059, 1196618, -, 1560, 856546; cds-WP_002910917.1, NZ_KK737546.1, 1197065, 1198036, +, 972, 2364158; cds-WP_002910918.1, NZ_KK737546.1, 1198093, 1198893, +, 801, 2331765; cds-WP_004145526.1, NZ_KK737546.1, 1198906, 1199757, +, 852, 2204752; cds-WP_002910920.1, NZ_KK737546.1, 1199820, 1200278, +, 459, 133571; cds-WP_009484665.1, NZ_KK737546.1, 1200697, 1201263, +, 567, 113729; cds-WP_002910922.1, NZ_KK737546.1, 1201267, 1202061, -, 795, 107700; cds-WP_002910924.1, NZ_KK737546.1, 1202123, 1203871, -, 1749, 264791; cds-WP_001062678.1, NZ_KK737546.1, 1204059, 1204268, -, 210, 1529418; cds-WP_071526670.1, NZ_KK737546.1, 1204351, 1204425, -, 75, 559602; cds-WP_002911374.1, NZ_KK737546.1, 1205016, 1205306, -, 291, 177976; cds-WP_002911375.1, NZ_KK737546.1, 1205381, 1205524, -, 144, 60709; cds-WP_004145533.1, NZ_KK737546.1, 1205684, 1205923, +, 240, 155909; cds-WP_002911378.1, NZ_KK737546.1, 1205980, 1206771, -, 792, 614517; cds-WP_072200054.1, NZ_KK737546.1, 1206898, 1208319, +, 1422, 75371; cds-WP_002911381.1, NZ_KK737546.1, 1208385, 1209269, -, 885, 2037296; cds-WP_004145536.1, NZ_KK737546.1, 1209505, 1211553, -, 2049, 3502314; cds-WP_002911385.1, NZ_KK737546.1, 1211573, 1212250, -, 678, 1229646; cds-WP_002911386.1, NZ_KK737546.1, 1212348, 1212845, -, 498, 138596; cds-WP_002911387.1, NZ_KK737546.1, 1212978, 1214261, +, 1284, 110841; cds-WP_002911388.1, NZ_KK737546.1, 1214230, 1216863, +, 2634, 930500; cds-WP_032429627.1, NZ_KK737546.1, 1216988, 1218421, +, 1434, 160902; cds-WP_002911393.1, NZ_KK737546.1, 1218537, 1218776, +, 240, 130994; cds-WP_002911395.1, NZ_KK737546.1, 1218874, 1219065, +, 192, 51; cds-WP_009484457.1, NZ_KK737546.1, 1219062, 1219715, -, 654, 15; cds-WP_014907334.1, NZ_KK737546.1, 1219872, 1220840, +, 969, 212057; cds-WP_002911398.1, NZ_KK737546.1, 1220945, 1221088, +, 144, 98019; cds-WP_002911404.1, NZ_KK737546.1, 1221724, 1222062, -, 339, 245869; cds-WP_032429628.1, NZ_KK737546.1, 1222079, 1222948, -, 870, 278904; cds-WP_004151448.1, NZ_KK737546.1, 1222952, 1223326, -, 375, 35547; cds-WP_002911406.1, NZ_KK737546.1, 1223439, 1223669, +, 231, 41108; cds-WP_002911407.1, NZ_KK737546.1, 1223748, 1224407, +, 660, 357376; cds-WP_004151449.1, NZ_KK737546.1, 1224411, 1226471, -, 2061, 478551; cds-WP_004180434.1, NZ_KK737546.1, 1226592, 1227251, -, 660, 434735; cds-WP_004184662.1, NZ_KK737546.1, 1227391,
1227738, -, 348, 80556; cds-WP_002911418.1, NZ_KK737546.1, 1227883, 1229061, +, 1179, 6539; cds-WP_002911423.1, NZ_KK737546.1, 1229119, 1229760, -, 642, 433668; cds-WP_002911425.1, NZ_KK737546.1, 1229799, 1231610, -, 1812, 100427; cds-WP_002911427.1, NZ_KK737546.1, 1231833, 1233308, -, 1476, 2242978; cds-WP _004145554.1, NZ_KK737546.1, 1233319, 1233468, -, 150, 295228; cds-WP_004145555.1, NZ_KK737546.1, 1233628, 1234533, +, 906, 662750; cds-WP_002911440.1, NZ_KK737546.1, 1234659, 1236101, +, 1443, 1727847; cds-WP _032429629.1, NZ_KK737546.1, 1236142, 1237116, -, 975, 163120; cds-WP_004148858.1, NZ_KK737546.1, 1237234, 1238553, -, 1320, 1351572; cds-WP _004180437.1, NZ_KK737546.1, 1238569, 1239513, -, 945, 648434; cds-WP _002911449.1, NZ_KK737546.1, 1239592, 1240344, +, 753, 126894; cds-WP_004212745.1, NZ_KK737546.1, 1240344, 1241129, +, 786, 25764; cds-WP _004148860.1, NZ_KK737546.1, 1241193, 1242203, -, 1011, 311948; cds-WP _002911454.1, NZ_KK737546.1, 1242212, 1242823, -, 612, 113180; cds-WP_002911456.1, NZ_KK737546.1, 1242903, 1243424, -, 522, 190997; cds-WP_002911459.1, NZ_KK737546.1, 1243459, 1244199, -, 741, 2268049; cds-WP_002911477.1, NZ_KK737546.1, 1244227, 1244670, -, 444, 393106; cds-WP_002911479.1, NZ_KK737546.1, 1244672, 1246459, -, 1788, 3121888; cds-WP_004145564.1, NZ_KK737546.1, 1246727, 1247293, +, 567, 299718; cds-WP_009307531.1, NZ_KK737546.1, 1247290, 1248108, +, 819, 82307; cds-WP _002911484.1, NZ_KK737546.1, 1248161, 1248556, +, 396, 82812; cds-WP_004175417.1, NZ_KK737546.1, 1248596, 1249339, +, 744, 245025; cds-WP_032429630.1, NZ_KK737546.1, 1249336, 1250340, +, 1005, 486093; cds-WP _032429631.1, NZ_KK737546.1, 1250422, 1251165, -, 744, 325423; cds-WP _015958659.1, NZ_KK737546.1, 1251242, 1251811, -, 570, 1230; cds-WP_004151452.1, NZ_KK737546.1, 1252047, 1253780, +, 1734, 1212075; cds-WP _002911497.1, NZ_KK737546.1, 1253842, 1254981, -, 1140, 22011; cds-WP _021440394.1, NZ_KK737546.1, 1254986, 1256569, -, 1584, 0; cds-WP _002911500.1, NZ_KK737546.1, 1256923, 1257351, +, 429, 29911; cds-WP _004175414.1, NZ_KK737546.1, 1257375, 1258799, -, 1425, 154473; cds-WP _002911505.1, NZ_KK737546.1, 1258774, 1259562, -, 789, 14368; cds-WP_002911507.1, NZ_KK737546.1, 1259724, 1260704, -, 981, 11322; cds-WP_004184670.1, NZ_KK737546.1, 1260719, 1262233, -, 1515, 4751; cds-WP _009484443.1, NZ_KK737546.1, 1262296, 1263276, -, 981, 530; cds-WP_160540596.1, NZ _KK737546.1, 1263638, 1263736, +, 99, 0; cds-WP_004175413.1, NZ_KK737546.1, 1264168, 1264677, +, 510, 40275; cds-WP_004148869.1, NZ_KK737546.1, 1264748, 1264906, -, 159, 65; cds-WP_002911522.1, NZ_KK737546.1, 1264972, 1266483, +, 1512, 73967; cds-WP_002911524.1, NZ_KK737546.1, 1266521, 1266772, -, 252, 42929; cds-WP_021440395.1, NZ_KK737546.1, 1266923, 1268344, +, 1422, 12522; cds-WP_021440396.1, NZ_KK737546.1, 1268394, 1269032, -, 639, 74378; cds-AF52_RS0123570, NZ_KK737546.1, 1269204, 1269384, +, 181, 0; cds-WP_004141101.1, NZ_KK737546.1, 1269442, 1269939, +, 498, 206156; cds-WP_024622766.1, NZ_KK737546.1, 1269975, 1270214, -, 240, 17841; cds-WP _004151453.1, NZ_KK737546.1, 1270406, 1271617, +, 1212, 127232; cds-WP_004141106.1, NZ_KK737546.1, 1271642, 1272310, -, 669, 102824; cds-WP_004180444.1, NZ_KK737546.1, 1272409, 1273290, -, 882, 21839; cds-WP_002911537.1, NZ_KK737546.1, 1273958, 1274506, -, 549, 54323; cds-WP _002911538.1, NZ_KK737546.1, 1274564, 1276396, -, 1833, 566251; cds-WP_002911539.1, NZ_KK737546.1, 1276393, 1277049, -, 657, 111855; cds-WP_002911541.1, NZ_KK737546.1, 1277511, 1277735, +, 225, 83847; cds-WP_032429632.1, NZ_KK737546.1, 1277803, 1278525, -, 723, 647671; cds-WP_024622767.1, NZ_KK737546.1, 1278843, 1279595, -, 753, 49226; cds-WP_002911547.1, NZ_KK737546.1, 1279592, 1280260, -, 669, 356050; cds-WP _021313403.1, NZ_KK737546.1, 1280276, 1281262, -, 987, 257269; cds-WP _004141135.1, NZ_KK737546.1, 1281358, 1282158, -, 801, 273068; cds-WP_004899206.1, NZ_KK737546.1, 1282359, 1283846, +, 1488,365360; cds-WP_004189223.1, NZ_KK737546.1, 1283888, 1284322, -, 435, 19280; cds-WP_004180447.1, NZ_KK737546.1, 1284695, 1285318, +, 624, 234843; cds-WP_002911586.1, NZ_KK737546.1, 1285351, 1285542, -, 192, 64874; cds-WP_002911589.1, NZ_KK737546.1, 1285676, 1285897, +, 222, 263; cds-WP_013815099.1-26, NZ_KK737546.1, 1286063, 1287031, -, 969, 106043; cds-AF52_RS25350, NZ_KK737546.1, 1287063, 1287197, -, 135, 575; cds-WP _004184683.1, NZ_KK737546.1, 1287375, 1288286, +, 912, 53524; cds-WP_024622769.1, NZ_KK737546.1, 1288261, 1288755, -, 495, 41563; cds-WP_004153246.1, NZ_KK737546.1, 1288736, 1290136, -, 1401, 115612; cds-WP_002911594.1, NZ_KK737546.1, 1290213, 1290920, -, 708, 45503; cds-WP_004151461.1, NZ_KK737546.1, 1290963, 1291244, -, 282, 9461; cds-WP _002911596.1, NZ_KK737546.1, 1291783, 1292928, +, 1146, 88322; cds-WP _004227143.1, NZ_KK737546.1, 1293381, 1294181, +, 801, 206942; cds-WP_009484428.1, NZ_KK737546.1, 1294767, 1295462, +, 696, 27021; cds-WP_016946077.1, NZ_KK737546.1, 1295493, 1295690, +, 198, 13612; cds-WP_022631352.1, NZ_KK737546.1, 1295840, 1296781, -, 942, 221391; cds-WP_004175385.1, NZ_KK737546.1, 1296860, 1297810, -, 951, 24362; cds-WP_002911729.1, NZ_KK737546.1, 1297917, 1298834, -, 918, 1483; cds-WP_004148971.1, NZ_KK737546.1, 1299334, 1300968, -, 1635, 48332; cds-WP _022631351.1, NZ_KK737546.1, 1301560, 1302264, +, 705, 65423; cds-WP_004151466.1, NZ_KK737546.1, 1302261, 1303238, +, 978, 42311; cds-WP_004184763.1, NZ_KK737546.1, 1303359, 1304291, +, 933, 23291; cds-WP_032429633.1, NZ_KK737546.1, 1304202, 1305824, -, 1623, 5997; cds-WP_004141189.1, NZ_KK737546.1, 1306097, 1306342, +, 246, 41059; cds-WP_004148975.1, NZ_KK737546.1, 1306472, 1307926, +, 1455, 647867; cds-WP _004151469.1, NZ_KK737546.1, 1308292, 1309728, -, 1437, 13548; cds-AF52_RS25355, NZ_KK737546.1, 1310050, 1310136, -, 87, 1862; cds-WP _022631349.1, NZ_KK737546.1, 1310278, 1311381, +, 1104, 38443; cds-WP_002911798.1, NZ_KK737546.1, 1311378, 1312349, -, 972, 31317; cds-WP _004180471.1, NZ_KK737546.1, 1312556, 1313392, -, 837, 101847; cds-WP_016532101.1, NZ_KK737546.1, 1313585, 1314160, -, 576, 476; cds-WP_022631348.1, NZ_KK737546.1, 1314292, 1315206, +, 915, 11079; cds-WP_002911862.1, NZ_KK737546.1, 1315212, 1315547, -, 336, 12948; cds-WP _002911866.1, NZ_KK737546.1, 1315554, 1315886, -, 333, 5953; cds-WP _004144110.1, NZ_KK737546.1, 1315984, 1316112, +, 129, 2154; cds-WP_016831855.1, NZ_KK737546.1, 1316109, 1318019, -, 1911, 104748; cds-WP _014907250.1, NZ_KK737546.1, 1318030, 1318491, -, 462, 128087; cds-WP _022631347.1, NZ_KK737546.1, 1318835, 1320154, +, 1320, 37573; cds-WP_022631346.1, NZ_KK737546.1, 1320201, 1320941, +, 741, 9198; cds-WP _032429739.1, NZ_KK737546.1, 1320901, 1321857, -, 957, 96524; cds-WP _004175282.1, NZ_KK737546.1, 1321973, 1322362, +, 390, 76337; cds-WP_002911879.1, NZ_KK737546.1, 1322384, 1323115, -, 732, 113225; cds-WP _004200363.1, NZ_KK737546.1, 1323314, 1324423, +, 1110, 286379; cds-WP_004200364.1, NZ_KK737546.1, 1324466, 1325533, -, 1068, 45509; cds-WP _021440419.1, NZ_KK737546.1, 1325561, 1326301, -, 741, 32188; cds-WP_002911883.1, NZ_KK737546.1, 1326616, 1326939, -, 324, 768700; cds-WP_032429634.1, NZ_KK737546.1, 1327107, 1328165, -, 1059, 644843; cds-WP _021440421.1, NZ_KK737546.1, 1328264, 1328737, -, 474, 567867; cds-WP_004153104.1, NZ_KK737546.1, 1328913, 1330076, -, 1164, 257567; cds-WP_032429635.1, NZ_KK737546.1, 1330257, 1331681, +, 1425, 290164; cds-WP_002912106.1, NZ_KK737546.1, 1331905, 1334007, -, 2103, 67589; cds-WP_002912109.1, NZ_KK737546.1, 1334247, 1334558, +, 312, 67117; cds-WP_004148994.1, NZ_KK737546.1, 1334740, 1336098, -, 1359, 833543; cds-WP _096335043.1, NZ_KK737546.1, 1336088, 1336150, -, 63, 22519; cds-WP _004175276.1, NZ_KK737546.1, 1336388, 1337278, -, 891, 26244; cds-WP _004152513.1, NZ_KK737546.1, 1337317, 1338141, -, 825, 57863; cds-WP_004899375.1, NZ_KK737546.1, 1338318, 1338368, +, 51, 10026; cds-WP _002912152.1, NZ_KK737546.1, 1338514, 1339413, +, 900, 162932; cds-WP _009307478.1, NZ_KK737546.1, 1339453, 1340757, +, 1305, 222518; cds-WP_032429636.1, NZ_KK737546.1, 1340754, 1341815, +, 1062, 191747; cds-WP_004144133.1, NZ_KK737546.1, 1341812, 1342879, +, 1068, 111285; cds-WP _002912227.1, NZ _KK737546.1, 1342879, 1343469, +, 591, 129433; cds-WP_004175272.1, NZ _KK737546.1, 1343469, 1344206, +, 738, 130620; cds-WP_004180497.1, NZ _KK737546.1, 1344188, 1344964, +, 777, 287876; cds-WP_032429637.1, NZ_KK737546.1, 1344958, 1345557, +, 600, 355077; cds-WP_004198893.1, NZ_KK737546.1, 1345750, 1346127, +, 378, 91346; cds-AF52_RS25360, NZ_KK737546.1, 1346124, 1346378, +, 255, 16194; cds-AF52_RS16545, NZ_KK737546.1, 1351106, 1351364, +, 259, 18616; cds-AF52_RS16540, NZ_KK737546.1, 1351435, 1352232, +, 798, 168178; cds-AF52_RS27905, NZ_KK737546.1, 1352265, 1352369, +, 105, 2218; cds-AF52_RS0123600, NZ_KK737546.1, 1352352, 1353032, +, 681, 193940; cds-AF52_RS0123605, NZ _KK737546.1, 1353102, 1353650, -, 549, 111817; cds-WP_013815099.1-27, NZ _KK737546.1, 1353685, 1354653, +, 969, 141110; cds-AF52_RS25365, NZ_KK737546.1, 1354708, 1355376, -, 669, 583060; cds-WP_072269281.1, NZ_KK737546.1, 1355472, 1356614, -, 1143, 1233442; cds-WP _032429639.1, NZ_KK737546.1, 1356615, 1357508, -, 894, 2252091; cds-WP_004890861.1, NZ_KK737546.1, 1357505, 1358659, -, 1155, 3100352; cds-WP_004214006.1, NZ_KK737546.1, 1358675, 1360570, -, 1896, 5136627; cds-WP _004175264.1, NZ_KK737546.1, 1360586, 1361326, -, 741, 889895; cds-WP_002912373.1, NZ_KK737546.1, 1361326, 1362093, -, 768, 924818; cds-WP_013815099.1-28, NZ_KK737546.1, 1362205, 1363173, +, 969, 231689; cds-WP _032429640.1, NZ_KK737546.1, 1364205, 1365209, +, 1005, 980506; cds-AF52_RS26960, NZ_KK737546.1, 1368345, 1368481, +, 137, 5942; cds-AF52_RS26965, NZ_KK737546.1, 1368526, 1368645, -, 120, 2202; cds-WP_032429643.1, NZ_KK737546.1, 1368690, 1368818, +, 129, 334; cds-WP_032429644.1, NZ_KK737546.1, 1369241, 1370407, -, 1167, 2984414; cds-WP _004899416.1, NZ_KK737546.1, 1370571, 1371941, -, 1371, 1627961; cds-WP _004180506.1, NZ_KK737546.1, 1371964, 1373379, -, 1416, 2796554; cds-WP _020802781.1, NZ_KK737546.1, 1373607, 1375013, -, 1407, 18049258; cds-WP_013815099.1-29, NZ_KK737546.1, 1375211, 1376179, -, 969, 130594; cds-WP _020802783.1, NZ_KK737546.1, 1376246, 1377643, -, 1398, 290143; cds-WP _032429645.1, NZ_KK737546.1, 1377862, 1378497, -, 636, 9017; cds-AF52_RS26980, NZ_KK737546.1, 1381542, 1381660, -, 119, 81482; cds-WP_072001573.1, NZ_KK737546.1, 1381682, 1382065, -, 384, 271607; cds-WP_032429646.1, NZ_KK737546.1, 1382073, 1383821, -, 1749, 1746484; cds-WP _020801666.1, NZ_KK737546.1, 1383842, 1384609, -, 768, 519511; cds-WP_020801633.1, NZ_KK737546.1, 1384619, 1385788, -, 1170, 927633; cds-WP_032429647.1, NZ_KK737546.1, 1385800, 1386888, -, 1089, 745825; cds-WP _032429746.1, NZ_KK737546.1, 1386888, 1388180, -, 1293, 529439; cds-WP _020801632.1, NZ_KK737546.1, 1388137, 1389291, -, 1155, 326178; cds-WP_020801659.1, NZ_KK737546.1, 1389302, 1390468, -, 1167, 609459; cds-WP_020801642.1, NZ_KK737546.1, 1390472, 1391422, -, 951, 857941; cds-WP_013815099.1-30, NZ_KK737546.1, 1391484, 1392452, +, 969, 245994; cds-WP_032429648.1, NZ_KK737546.1, 1392641, 1394812, -, 2172, 4029423; cds-WP _072001574.1, NZ_KK737546.1, 1394831, 1395262, -, 432, 1214349; cds-WP_020801619.1, NZ_KK737546.1, 1395269, 1396402, -, 1134, 3138056; cds-WP _032429649.1, NZ_KK737546.1, 1396548, 1397981, -, 1434, 1879818; cds-WP _032429650.1, NZ _KK737546.1, 1398272, 1398394, +, 123, 68814; cds-WP_020806188.1-2, NZ_KK737546.1, 1398736, 1399716, -, 981, 348459; cds-WP _004184823.1, NZ_KK737546.1, 1400141, 1400770, -, 630, 472747; cds-WP_001741945.1, NZ_KK737546.1, 1401163, 1402053, -, 891, 973226; cds-WP _004151147.1, NZ_KK737546.1, 1402818, 1404401, +, 1584, 323106; cds-WP_032412678.1, NZ_KK737546.1, 1404896, 1406743, -, 1848, 416214; cds-WP_004151145.1, NZ_KK737546.1, 1406774, 1407355, -, 582, 285730; cds-WP _002912442.1, NZ_KK737546.1, 1407446, 1408087, -, 642, 942516; cds-WP _032429651.1, NZ_KK737546.1, 1408202, 1409050, -, 849, 40077; cds-WP_032429652.1, NZ_KK737546.1, 1409188, 1410540, +, 1353, 51574; cds-WP _032429747.1, NZ_KK737546.1, 1410830, 1412686, +, 1857, 82379; cds-WP_032429653.1, NZ_KK737546.1, 1412701, 1413402, +, 702, 7273; cds-WP_032409828.1, NZ_KK737546.1, 1413640, 1413780, +, 141, 5453; cds-WP_004899454.1, NZ_KK737546.1, 1413896, 1414507, +, 612, 29871; cds-WP _004149053.1, NZ _KK737546.1, 1414781, 1416055, -, 1275, 59949; cds-WP_032429654.1, NZ_KK737546.1, 1416356, 1417594, +, 1239, 12333; cds-WP _004149055.1, NZ_KK737546.1, 1417594,
1420716, +, 3123, 254416; cds-WP_032429655.1, NZ_KK737546.1, 1420717, 1423794, +, 3078, 286474; cds-WP_032429656.1, NZ_KK737546.1, 1423796, 1425211, +, 1416, 6738; cds-WP_004149058.1, NZ_KK737546.1, 1425208, 1426686, +, 1479, 9506; cds-WP_004175198.1, NZ_KK737546.1, 1426683, 1427405, +, 723, 42123; cds-WP _004144192.1, NZ_KK737546.1, 1427724, 1429085, +, 1362, 88078; cds-WP_004151134.1, NZ_KK737546.1, 1429328, 1430224, +, 897, 158983; cds-WP_032429657.1, NZ_KK737546.1, 1430465, 1431238, -, 774, 1130; cds-WP_032429748.1, NZ_KK737546.1, 1431249, 1432112, -, 864, 32673; cds-WP _004200464.1, NZ_KK737546.1, 1432084, 1432989, -, 906, 0; cds-WP_004149062.1, NZ_KK737546.1, 1432986, 1434023, -, 1038, 1569; cds-WP _004180553.1, NZ_KK737546.1, 1434020, 1435642, -, 1623, 5672; cds-WP _002912648.1, NZ_KK737546.1, 1435762, 1436916, -, 1155, 69259; cds-WP _032429658.1, NZ_KK737546.1, 1437214, 1437876, -, 663, 329; cds-WP _016529109.1, NZ_KK737546.1, 1437925, 1439202, -, 1278, 1983; cds-WP_032429659.1, NZ_KK737546.1, 1439273, 1440736, -, 1464, 2990; cds-WP_004144197.1, NZ_KK737546.1, 1440746, 1442113, -, 1368, 75466; cds-WP_004144198.1, NZ_KK737546.1, 1442320, 1443261, +, 942, 104278; cds-WP_032429660.1, NZ_KK737546.1, 1443284, 1444285, -, 1002, 41144; cds-WP _004180592.1, NZ_KK737546.1, 1444540, 1445289, +, 750, 3305; cds-WP_023285368.1, NZ_KK737546.1, 1445316, 1446923, +, 1608, 8240; cds-WP _004175116.1, NZ_KK737546.1, 1447005, 1448288, +, 1284, 18180; cds-WP _002912704.1, NZ_KK737546.1, 1448347, 1449399, -, 1053, 223502; cds-WP _002912707.1, NZ_KK737546.1, 1449547, 1450347, -, 801, 16044; cds-WP_004151131.1, NZ_KK737546.1, 1450344, 1451117, -, 774, 107383; cds-WP_009307427.1, NZ_KK737546.1, 1451435, 1451857, +, 423, 32804; cds-WP _004175111.1, NZ_KK737546.1, 1451854, 1452768, -, 915, 15306; cds-WP _009307426.1, NZ_KK737546.1, 1452889, 1454700, +, 1812, 461; cds-WP_009307425.1, NZ_KK737546.1, 1454703, 1456046, +, 1344, 584; cds-WP _004149074.1, NZ_KK737546.1, 1456036, 1456935, -, 900, 5333; cds-WP _002912749.1, NZ_KK737546.1, 1457119, 1457436, +, 318, 7186; cds-WP_072269284.1, NZ_KK737546.1, 1457408, 1457872, +, 465, 36940; cds-WP _032429661.1, NZ_KK737546.1, 1457869, 1458978, -, 1110, 404269; cds-WP _002912753.1, NZ_KK737546.1, 1459132, 1461165, +, 2034, 1928940; cds-WP_002912756.1, NZ_KK737546.1, 1461280, 1461750, -, 471, 97365; cds-WP_004144215.1, NZ_KK737546.1, 1461798, 1462517, -, 720, 75206; cds-WP_002912760.1, NZ_KK737546.1, 1462511, 1464199, -, 1689, 287113; cds-WP_002912762.1, NZ_KK737546.1, 1464429, 1464542, +, 114, 7769; cds-WP_022631308.1, NZ_KK737546.1, 1464517, 1465254, -, 738, 65956; cds-WP _022631307.1, NZ_KK737546.1, 1465238, 1466185, -, 948, 13847; cds-WP _023285369.1, NZ_KK737546.1, 1466178, 1467335, -, 1158, 22797; cds-WP_002912823.1, NZ_KK737546.1, 1467343, 1468260, -, 918, 35998; cds-WP_009307419.1, NZ_KK737546.1, 1468397, 1470694, -, 2298,580783; cds-WP_002912827.1, NZ_KK737546.1, 1470889, 1472634, +, 1746, 1879089; cds-WP_002912829.1, NZ _KK737546.1, 1472720, 1473274, +, 555, 70578; cds-WP_004180615.1, NZ_KK737546.1, 1473497, 1474447, -, 951, 205716; cds-WP_002912831.1, NZ_KK737546.1, 1474636, 1475223, -, 588, 40401; cds-WP _009307418.1, NZ _KK737546.1, 1475378, 1475947, +, 570, 19616; cds-WP _032429662.1, NZ_KK737546.1, 1475981, 1477420, -, 1440, 134356; cds-WP _022631304.1, NZ_KK737546.1, 1477692, 1479086, -, 1395, 58013; cds-WP_032429663.1, NZ_KK737546.1, 1479102, 1480964, -, 1863, 143583; cds-WP_002912842.1, NZ_KK737546.1, 1481107, 1481940, -, 834, 122151; cds-WP_004175080.1, NZ_KK737546.1, 1482096, 1483460, -, 1365, 39007; cds-WP_002912862.1, NZ_KK737546.1, 1483817, 1484749, -, 933, 40948; cds-WP _002912865.1, NZ_KK737546.1, 1485107, 1485505, +, 399, 9176; cds-WP_002912867.1, NZ_KK737546.1, 1485502, 1486197, +, 696, 14086; cds-WP _002912869.1, NZ_KK737546.1, 1486325, 1487209, +, 885, 53082; cds-WP _004211527.1, NZ_KK737546.1, 1487485, 1488201, +, 717, 88470; cds-WP_002912871.1, NZ_KK737546.1, 1488272, 1489282, -, 1011, 1164934; cds-WP_004175074.1, NZ_KK737546.1, 1489298, 1490818, -, 1521, 5497921; cds-WP_002912878.1, NZ_KK737546.1, 1490937, 1491935, -, 999, 8270135; cds-WP _002912919.1, NZ_KK737546.1, 1492232, 1493254, -, 1023, 1235678; cds-WP _021440476.1, NZ_KK737546.1, 1493410, 1494567, -, 1158, 154646; cds-WP_002912924.1, NZ_KK737546.1, 1494583,1495251, -, 669, 1228762; cds-WP_004151123.1, NZ_KK737546.1, 1495612, 1496445, +, 834, 52046; cds-WP_032429664.1, NZ_KK737546.1, 1496516, 1498489, -, 1974, 394780; cds-WP_002912929.1, NZ_KK737546.1, 1498988, 1500457, -, 1470, 370398; cds-WP _002912932.1, NZ_KK737546.1, 1500636, 1501502, -, 867, 36115; cds-WP_002912935.1, NZ_KK737546.1, 1501768, 1502817, +, 1050, 9504; cds-WP_002912937.1, NZ_KK737546.1, 1502890, 1503744, +, 855, 461314; cds-WP _021440480.1, NZ_KK737546.1, 1503795, 1505489, -, 1695, 507455; cds-WP_002912941.1, NZ_KK737546.1, 1505506, 1506444, -, 939, 233183; cds-WP_032428355.1, NZ_KK737546.1, 1506445, 1507575, -, 1131, 164545; cds-WP_032429665.1, NZ_KK737546.1, 1507911, 1509092, +, 1182, 110886; cds-WP_004175066.1, NZ_KK737546.1, 1509132, 1509386, -, 255, 79389; cds-WP_002912951.1, NZ_KK737546.1, 1509534, 1510106, +, 573, 1023021; cds-WP_002912952.1, NZ_KK737546.1, 1510177, 1511367, -, 1191, 180523; cds-WP _004200498.1, NZ_KK737546.1, 1511575, 1513041, +, 1467, 584617; cds-WP_021463904.1, NZ_KK737546.1, 1513162, 1514139, +, 978, 3681; cds-WP_072269286.1, NZ_KK737546.1, 1514177, 1514890, +, 714, 212062; cds-WP_002912967.1, NZ_KK737546.1, 1515317, 1515886, +, 570, 555550; cds-WP_021440487.1, NZ_KK737546.1, 1516075, 1517637, +, 1563, 90033; cds-WP_032429666.1, NZ_KK737546.1, 1517706, 1519511, +, 1806, 35953; cds-WP_004200501.1, NZ_KK737546.1, 1519521, 1520615, +, 1095, 63485; cds-WP _004149131.1, NZ_KK737546.1, 1520615, 1521640, +, 1026, 150414; cds-WP _004151118.1, NZ_KK737546.1, 1521642, 1523231, +, 1590, 469776; cds-WP _002912974.1, NZ_KK737546.1, 1523235, 1523579, -, 345, 245751; cds-WP _002912975.1, NZ_KK737546.1, 1523911, 1525107, -, 1197, 237418; cds-WP _002912977.1, NZ_KK737546.1, 1525104, 1525823, -, 720, 205172; cds-WP _004144263.1, NZ_KK737546.1, 1525971, 1527728, +, 1758, 208464; cds-WP_002912979.1, NZ_KK737546.1, 1527865, 1528149, +, 285, 573763; cds-WP_004189004.1, NZ_KK737546.1, 1528208, 1529095, -, 888, 201346; cds-AF52_RS0123640, NZ_KK737546.1, 1529200, 1530485, +, 1286, 8164; cds-WP _024622810.1, NZ_KK737546.1, 1530504, 1531100, -, 597, 2121; cds-WP_002912986.1, NZ_KK737546.1, 1531183, 1531944, +, 762, 68058; cds-WP_004144267.1, NZ_KK737546.1, 1531964, 1532971, -, 1008,353952; cds-WP_004195346.1, NZ_KK737546.1, 1533158, 1533385, +, 228,37995; cds-WP_002912990.1, NZ_KK737546.1, 1533404, 1535164, +, 1761, 1309564; cds-WP_032429667.1, NZ_KK737546.1, 1535502, 1536611, +, 1110, 84771; cds-WP_032428356.1, NZ_KK737546.1, 1536786, 1537277, +, 492, 3985337; cds-WP _002912994.1, NZ _KK737546.1, 1537344, 1538978, -, 1635, 254156; cds-AF52_RS0123645, NZ _KK737546.1, 1539025, 1539722, -, 698, 451234; cds-WP_020324457.1, NZ _KK737546.1, 1540115, 1541275, +, 1161, 210434; cds-WP _002912996.1, NZ_KK737546.1, 1541238, 1542674, -, 1437, 81754; cds-WP _004184919.1, NZ_KK737546.1, 1542823, 1544466, -, 1644, 225620; cds-WP_016530253.1, NZ_KK737546.1, 1544541, 1545194, -, 654, 15227; cds-WP_021440495.1, NZ_KK737546.1, 1545194, 1546258, -, 1065, 35048; cds-WP _032429668.1, NZ_KK737546.1, 1546332, 1547384, -, 1053, 279985; cds-WP_032429669.1, NZ_KK737546.1, 1547488, 1548582, -, 1095, 20381269; cds-WP_002913006.1, NZ_KK737546.1, 1549354, 1552011, +, 2658, 1098779; cds-WP_002913007.1, NZ_KK737546.1, 1552027, 1552677, +, 651, 688409; cds-WP _032429670.1, NZ_KK737546.1, 1552723, 1555563, -, 2841, 473899; cds-WP_032429671.1, NZ_KK737546.1, 1555695, 1558328, -, 2634, 3845578; cds-WP_002913014.1, NZ_KK737546.1, 1558475, 1559203, +, 729, 93090; cds-WP_002913016.1, NZ_KK737546.1, 1559548, 1561833, +, 2286, 8482742; cds-WP _004140835.1, NZ_KK737546.1, 1561935, 1563065, +, 1131, 2586750; cds-WP _002913017.1, NZ_KK737546.1, 1563065, 1563319, +, 255, 47576; cds-WP_032429672.1, NZ_KK737546.1, 1563783, 1564853, -, 1071, 5956593; cds-WP_002913019.1, NZ_KK737546.1, 1564863, 1566209, -, 1347, 5352892; cds-WP_004214634.1, NZ_KK737546.1, 1566481, 1568103, +, 1623, 3218; cds-WP_015958752.1, NZ_KK737546.1, 1568093, 1569352, +, 1260, 6448; cds-WP_004184966.1, NZ_KK737546.1, 1569349, 1570527, +, 1179, 128543; cds-WP_032428357.1, NZ_KK737546.1, 1570549, 1571352, -, 804, 8626; cds-WP _002913045.1, NZ_KK737546.1, 1571367, 1572656, -, 1290, 32948; cds-WP_004175029.1, NZ_KK737546.1, 1572699, 1573904, -, 1206, 5192; cds-WP_002913046.1, NZ_KK737546.1, 1573915, 1574700, -, 786, 2539; cds-WP_004184973.1, NZ_KK737546.1, 1574905, 1576101, -, 1197, 151671; cds-WP_019705497.1, NZ_KK737546.1, 1576204, 1576746, -, 543, 92788; cds-WP_024622815.1, NZ_KK737546.1, 1577021, 1577962, -, 942, 77846; cds-WP_032429673.1, NZ_KK737546.1, 1578100, 1578525, +, 426, 12955; cds-WP _021440500.1, NZ_KK737546.1, 1578581, 1579957, -, 1377, 30430; cds-WP _021440501.1, NZ_KK737546.1, 1579954, 1580919, -, 966, 285; cds-WP _002913098.1, NZ_KK737546.1, 1580919, 1581776, -, 858, 100045; cds-WP _021440502.1, NZ_KK737546.1, 1581791, 1582549, -, 759, 21627; cds-WP_002913103.1, NZ_KK737546.1, 1582546, 1584216, -, 1671, 20764; cds-WP_021440503.1, NZ_KK737546.1, 1584299, 1585588, -, 1290, 6713; cds-WP_004184987.1, NZ_KK737546.1, 1585724, 1586185, -, 462, 97739; cds-WP _004214593.1, NZ _KK737546.1, 1586243, 1587163, +, 921, 24691; cds-WP_004151107.1, NZ_KK737546.1, 1587218, 1588675, -, 1458, 1037504; cds-WP _032429674.1, NZ_KK737546.1, 1588682, 1590211, -, 1530, 2251171; cds-WP _032429675.1, NZ_KK737546.1, 1590381, 1592222, -, 1842, 2114408; cds-WP _002913148.1, NZ_KK737546.1, 1592219, 1592521, -, 303, 542349; cds-WP _002913150.1, NZ_KK737546.1, 1592518, 1593072, -, 555, 1533827; cds-WP_002913152.1, NZ_KK737546.1, 1593084, 1593626, -, 543, 1029777; cds-WP_002913156.1, NZ_KK737546.1, 1593641, 1594618, -, 978, 2419389; cds-WP_023285394.1, NZ_KK737546.1, 1594615, 1597341, -, 2727, 4677790; cds-WP _002913158.1, NZ_KK737546.1, 1597374, 1598711, -, 1338, 2656024; cds-WP _002913160.1, NZ_KK737546.1, 1598708, 1599208, -, 501, 1150177; cds-WP_002913162.1, NZ_KK737546.1, 1599211, 1601019, -, 1809, 7951496; cds-WP_002913178.1, NZ_KK737546.1, 1601106, 1601780, -, 675, 2168125; cds-WP _002913181.1, NZ_KK737546.1, 1601796, 1602242, -, 447, 1015210; cds-WP_021440508.1, NZ_KK737546.1, 1602894, 1603820, -, 927, 113926; cds-WP _002913185.1, NZ_KK737546.1, 1604693, 1605910, +, 1218, 702922; cds-WP_002913188.1, NZ_KK737546.1, 1605992, 1606591, +, 600, 154086; cds-WP _032428358.1, NZ_KK737546.1, 1606636, 1608468, -, 1833, 236088; cds-WP _019705510.1, NZ_KK737546.1, 1608545, 1609204, -, 660, 202494; cds-WP_002913193.1, NZ_KK737546.1, 1609216, 1609710, -, 495, 184865; cds-WP_002913194.1, NZ_KK737546.1, 1609798, 1610253, -, 456, 57638; cds-WP _002913195.1, NZ_KK737546.1, 1610591, 1611793, +, 1203, 5459924; cds-WP _002913196.1, NZ_KK737546.1, 1611994, 1614141, +, 2148, 8670279; cds-WP_032429676.1, NZ_KK737546.1, 1614234, 1614785, -, 552, 167200; cds-WP _004174991.1, NZ_KK737546.1, 1614843, 1615394, -, 552, 332685; cds-WP_004180746.1, NZ_KK737546.1, 1615457, 1616095, -, 639, 96601; cds-WP_004174988.1, NZ_KK737546.1, 1616276, 1616905, +, 630, 15222; cds-WP _002913200.1, NZ _KK737546.1, 1616971, 1617333, +, 363, 358484; cds-WP_002913203.1, NZ _KK737546.1, 1617352, 1618245, +, 894, 657420; cds-WP_004217247.1, NZ _KK737546.1, 1618225, 1618341, -, 117, 26237; cds-WP _032429677.1, NZ_KK737546.1, 1618461, 1618988, +, 528, 115641; cds-WP_002913206.1, NZ_KK737546.1, 1618994, 1619767, -, 774, 218155; cds-WP_002913207.1, NZ_KK737546.1, 1619775, 1620491, -, 717, 310020; cds-WP_002913208.1, NZ_KK737546.1, 1620488, 1621174, -, 687, 113593; cds-WP _002913209.1, NZ_KK737546.1, 1621239, 1622021, -, 783, 346869; cds-WP_002913210.1, NZ_KK737546.1, 1622289, 1623071, -, 783, 566324; cds-WP_004140952.1, NZ_KK737546.1, 1623117, 1623278, +, 162, 84026; cds-WP _014599227.1, NZ_KK737546.1, 1623359, 1623928, -, 570, 12650; cds-WP _032429678.1, NZ_KK737546.1, 1624064, 1625581, -, 1518, 1035331; cds-WP_002913214.1, NZ_KK737546.1, 1625615, 1626103, -, 489, 412096; cds-WP_004151102.1, NZ_KK737546.1, 1626320, 1627009, -, 690, 145164; cds-WP _021440515.1, NZ_KK737546.1, 1626999, 1628267, -, 1269, 293090; cds-WP_002913220.1, NZ_KK737546.1, 1628343, 1629263, -, 921, 862372; cds-WP_002913221.1, NZ_KK737546.1, 1629449,
1630108, -, 660, 547236; cds-WP_002913226.1, NZ_KK737546.1, 1630135, 1630947, -, 813, 456700; cds-WP_002913227.1, NZ_KK737546.1, 1630947, 1631960, -, 1014, 382869; cds-WP_021440517.1, NZ_KK737546.1, 1632024, 1633160, -, 1137, 213714; cds-WP_032429679.1, NZ_KK737546.1, 1633271, 1634248, +, 978, 71752; cds-WP_004149211.1, NZ_KK737546.1, 1634335, 1635510, -, 1176, 5016; cds-WP_002913291.1, NZ_KK737546.1, 1635720, 1636940, -, 1221, 909813; cds-WP_032429751.1, NZ_KK737546.1, 1637099, 1639087, +, 1989, 73970; cds-WP_002913338.1, NZ_KK737546.1, 1639149, 1639430, -, 282, 114554; cds-WP_032429680.1, NZ_KK737546.1, 1639462, 1640010, -, 549, 314494; cds-WP_002913340.1, NZ_KK737546.1, 1640010, 1640819, -, 810, 136632; cds-WP_0041749601, NZ_KK737546.1, 1640819, 1641643, -, 825, 884971; cds-WP_002913342.1, NZ_KK737546.1, 1641646, 1642731, -, 1086, 441138; cds-WP_002913346.1, NZ_KK737546.1, 1642773, 1643705, -, 933, 410804; cds-WP_002913348.1, NZ_KK737546.1, 1643873, 1644424, +, 552, 251769; cds-WP_002913355.1, NZ_KK737546.1, 1644445, 1644930, -, 486, 735571; cds-WP_032429681.1, NZ_KK737546.1, 1645140, 1647284, -, 2145, 1716483; cds-WP_002913358.1, NZ_KK737546.1, 1647284, 1648594, -, 1311, 1076943; cds-WP_002913359.1, NZ_KK737546.1, 1648754, 1649038, -, 285, 72284; cds-WP_002913360.1, NZ_KK737546.1, 1649412, 1650734, +, 1323, 1220127; cds-WP_002913362.1, NZ_KK737546.1, 1650796, 1651557, -, 762, 475817; cds-WP_004149224.1, NZ_KK737546.1, 1651847, 1652776, +, 930, 21191; cds-WP_004174945.1, NZ_KK737546.1, 1653189, 1653671, +, 483, 171605; cds-WP_032428359.1, NZ_KK737546.1, 1654039, 1654920, +, 882, 53558; cds-WP_021440524.1, NZ_KK737546.1, 1654930, 1655838, -, 909, 5876; cds-WP_009486224.1, NZ_KK737546.1, 1655971, 1656429, +, 459, 748; cds-WP_023282888.1, NZ_KK737546.1, 1656426, 1657622, +, 1197, 54839; cds-WP_099119320.1, NZ_KK737546.1, 1657961, 1658032, +, 72, 11865; cds-WP_002913372.1, NZ_KK737546.1, 1658103, 1659317, -, 1215, 184501; cds-WP_004188912.1, NZ_KK737546.1, 1659406, 1659591, +, 186, 94; cds-WP_004153200.1, NZ_KK737546.1, 1659588, 1659701, -, 114, 1; cds-WP_004149230.1, NZ_KK737546.1, 1659700, 1661397, +, 1698, 132432; cds-WP_002913374.1, NZ_KK737546.1, 1661409, 1662146, +, 738, 94035; cds-WP_002913377.1, NZ _KK737546.1, 1662200, 1663165, -, 966, 492627; cds-WP_004154521.1, NZ_KK737546.1, 1663429, 1664664, +, 1236, 78732; cds-WP_032428368.1, NZ_KK737546.1, 1664661, 1666322, -, 1662, 2473; cds-WP_002913417.1, NZ_KK737546.1, 1666503, 1667495, +, 993, 555624; cds-WP_002913419.1, NZ_KK737546.1, 1667626, 1667985, +, 360, 26832; cds-WP_002913421.1, NZ_KK737546.1, 1668043, 1669284, -, 1242, 55976; cds-WP_002913423.1, NZ_KK737546.1, 1669631, 1670833, +, 1203, 1130101; cds-WP_032429682.1, NZ_KK737546.1, 1670882, 1673053, -, 2172, 54710; cds-WP_002913434.1, NZ_KK737546.1, 1673558, 1673911, +, 354, 12081; cds-WP_002913435.1, NZ_KK737546.1, 1673915, 1674307, +, 393, 54256; cds-WP_004145598.1, NZ_KK737546.1, 1674359, 1675777, -, 1419, 2171725; cds-WP_021440528.1, NZ_KK737546.1, 1676741, 1677667, -, 927, 243197; cds-WP_021440529.1, NZ_KK737546.1, 1677756, 1678754, +, 999, 280663; cds-WP_002913440.1, NZ_KK737546.1, 1678751, 1678969, -, 219, 75410; cds-WP_004180820.1, NZ_KK737546.1, 1678971, 1680986, -, 2016, 187688; cds-WP_004180822.1, NZ_KK737546.1, 1681056, 1682117, -, 1062, 426485; cds-WP_004174922.1, NZ_KK737546.1, 1682348, 1683109, +, 762, 189855; cds-WP_002913498.1, NZ_KK737546.1, 1683286, 1684257, +, 972, 388795; cds-WP_002913505.1, NZ_KK737546.1, 1684638, 1684895, +, 258, 2073672; cds-WP_021440531.1, NZ_KK737546.1, 1684940, 1686667, +, 1728, 9211480; cds-WP_000522253.1, NZ_KK737546.1, 1686710, 1687219, +, 510, 2727897; cds-WP_004890526.1, NZ_KK737546.1, 1687261, 1687500, -, 240, 15477; cds-WP_004890524.1, NZ_KK737546.1, 1687497, 1688363, -, 867, 4994; cds-WP_004174915.1, NZ_KK737546.1, 1688446, 1689744, +, 1299, 35406; cds-WP_004890521.1, NZ_KK737546.1, 1689885, 1690280, +, 396, 34883; cds-WP_004890515.1, NZ_KK737546.1, 1690277, 1691188, -, 912, 79384; cds-WP_002913623.1, NZ_KK737546.1, 1691307, 1692401, -, 1095, 60166; cds-WP_004890510.1, NZ_KK737546.1, 1692391, 1693266, -, 876, 36145; cds-WP_002913626.1, NZ_KK737546.1, 1693266, 1694099, -, 834, 79208; cds-WP_032429684.1, NZ_KK737546.1, 1694099, 1695121, -, 1023, 28242; cds-AF52_RS25445, NZ_KK737546.1, 1695322, 1695665, +, 344, 23988; cds-WP_020803947.1, NZ_KK737546.1, 1695761, 1696660, -, 900, 487147; cds-WP_002913630.1, NZ_KK737546.1, 1696755, 1697330, -, 576, 303648; cds-WP_002913635.1, NZ_KK737546.1, 1697392, 1697841, -, 450, 45986; cds-WP_002913637.1, NZ_KK737546.1, 1697828, 1698253, -, 426, 124078; cds-WP_002913639.1, NZ_KK737546.1, 1698465, 1699337, +, 873, 892736; cds-WP_032429685.1, NZ_KK737546.1, 1699337, 1700236, +, 900, 366182; cds-WP_002913642.1, NZ_KK737546.1, 1700280, 1701332, -, 1053, 115365; cds-WP_032428360.1, NZ_KK737546.1, 1701378, 1701863, -, 486, 2645; cds-WP_004174905.1, NZ_KK737546.1, 1701875, 1702534, -, 660, 8092; cds-WP_021440537.1, NZ_KK737546.1, 1702544, 1703443, -, 900, 1877; cds-WP_032429686.1, NZ_KK737546.1, 1703464, 1704825, -, 1362, 4542; cds-WP_021440539.1, NZ_KK737546.1, 1704837, 1706240, -, 1404,32; cds-WP_004149260.1, NZ_KK737546.1, 1706237, 1707469, -, 1233, 17; cds-WP_032429687.1, NZ_KK737546.1, 1707669, 1708856, -, 1188, 0; cds-WP_004180842.1, NZ_KK737546.1, 1708846, 1709685, -, 840, 0; cds-WP_004152003.1, NZ_KK737546.1, 1709695, 1711098, -, 1404, 248; cds-WP_002913719.1, NZ_KK737546.1, 1711110, 1711397, -, 288, 93; cds-WP_000387720.1, NZ_KK737546.1, 1711526, 1711816, -, 291, 23; cds-WP_021440542.1, NZ_KK737546.1, 1711855, 1712871, -, 1017, 267; cds-WP_004180847.1, NZ_KK737546.1, 1712861, 1713667, -, 807, 26750; cds-WP_002913725.1, NZ_KK737546.1, 1713664, 1714353, -, 690, 35635; cds-WP_021440543.1, NZ_KK737546.1, 1714331, 1714810, -, 480, 0; cds-WP_002913729.1, NZ_KK737546.1, 1714823, 1715158, -, 336, 243; cds-WP_004185057.1, NZ_KK737546.1, 1715377, 1717656, -, 2280, 1193846; cds-WP_002913732.1, NZ_KK737546.1, 1717951, 1718907, +, 957, 117788; cds-WP_004149269.1, NZ_KK737546.1, 1718924, 1720918, +, 1995, 420775; cds-WP_009309515.1, NZ_KK737546.1, 1720908, 1721954, -, 1047, 39107; cds-WP_002913754.1, NZ_KK737546.1, 1722018, 1722614, -, 597, 111588; cds-WP_032429689.1, NZ_KK737546.1, 1722673, 1724655, -, 1983, 2332; cds-WP_004185072.1, NZ_KK737546.1, 1725028, 1728141, +, 3114, 186241; cds-WP_100181659.1, NZ_KK737546.1, 1728269, 1728328, -, 60, 332512; cds-WP_002913764.1, NZ_KK737546.1, 1728694, 1729047, +, 354, 46452; cds-WP_021440546.1, NZ_KK737546.1, 1729051, 1730178, +, 1128, 331260; cds-WP_002913768.1, NZ_KK737546.1, 1730205, 1730408, +, 204, 170347; cds-WP_002913770.1, NZ_KK737546.1, 1730457, 1731152, -, 696, 49633; cds-WP_021440547.1, NZ_KK737546.1, 1731229, 1733232, -, 2004, 106394; cds-WP_032429690.1, NZ_KK737546.1, 1733242, 1734117, -, 876, 77201; cds-WP_002913801.1, NZ_KK737546.1, 1734237, 1734950, -, 714, 646693; cds-WP_004149279.1, NZ_KK737546.1, 1735167, 1736201, -, 1035, 872043; cds-WP_002913803.1, NZ_KK737546.1, 1736218, 1737096, -, 879, 2822910; cds-WP_002913804.1, NZ_KK737546.1, 1737250, 1737816, +, 567, 417123; cds-WP_002913805.1, NZ_KK737546.1, 1737820, 1738290, +, 471, 532898; cds-WP_002913806.1, NZ_KK737546.1, 1738352, 1739413, -, 1062, 248952; cds-WP_004221267.1, NZ_KK737546.1, 1739468, 1739584, -, 117, 3898; cds-WP_002913807.1, NZ_KK737546.1, 1739636, 1741099, +, 1464, 654058; cds-WP_002913810.1, NZ_KK737546.1, 1741109, 1741468, +, 360, 55418; cds-WP_023301620.1, NZ_KK737546.1, 1741596, 1742519, +, 924, 229413; cds-WP_020947395.1, NZ_KK737546.1, 1742516, 1743217, -, 702, 168705; cds-WP_002913824.1, NZ_KK737546.1, 1743316, 1744602, -, 1287, 309755; cds-WP_002913827.1, NZ_KK737546.1, 1744698, 1745324, -, 627, 462095; cds-WP 021440551.1, NZ_KK737546.1, 1745542, 1746975, -, 1434, 158141; cds-WP_021440552.1, NZ_KK737546.1, 1746985, 1747878, -, 894, 3483; cds-WP_004149288.1, NZ_KK737546.1, 1748142, 1749179, +, 1038, 87988; cds-WP_004145656.1, NZ_KK737546.1, 1749176, 1749817, +, 642, 184624; cds-WP_002913838.1, NZ_KK737546.1, 1749998, 1752058, +, 2061, 1139557; cds-WP_002913839.1, NZ _KK737546.1, 1752062, 1753594, +, 1533, 415855; cds-WP_032429691.1, NZ_KK737546.1, 1753648, 1755876, -, 2229,36138; cds-WP_004174861.1, NZ_KK737546.1, 1756247, 1756420, +, 174, 10998; cds-WP_004221278.1, NZ_KK737546.1, 1756517, 1757428, -, 912, 26098; cds-WP_021440556.1, NZ_KK737546.1, 1757502, 1758734, +, 1233, 131304; cds-WP_021440557.1, NZ_KK737546.1, 1759028, 1760206, +, 1179, 10614; cds-WP_009309501.1, NZ_KK737546.1, 1760190, 1762058, +, 1869, 32755; cds-WP_004151979.1, NZ_KK737546.1, 1762153, 1763730, -, 1578, 1688331; cds-WP_004151980.1, NZ_KK737546.1, 1763798, 1765264, -, 1467, 1035553; cds-WP_015958798.1, NZ_KK737546.1, 1765423, 1766814, +, 1392, 201938; cds-WP_004144303.1, NZ_KK737546.1, 1766798, 1767016, -, 219, 7702; cds-WP_021313079.1, NZ_KK737546.1, 1767066, 1768145, -, 1080, 15051; cds-WP_020317242.1, NZ_KK737546.1, 1768329, 1768808, -, 480, 6422; cds-WP_032418518.1, NZ_KK737546.1, 1769030, 1769722, -, 693, 13944; cds-WP_004144309.1, NZ_KK737546.1, 1770089, 1771567, -, 1479, 659840; cds-WP_002913889.1, NZ_KK737546.1, 1771681, 1772859, -, 1179, 1212608; cds-WP_002913890.1, NZ_KK737546.1, 1772870, 1773490, -, 621, 1257848; cds-WP_004149335.1, NZ_KK737546.1, 1773525, 1774799, -, 1275, 1136210; cds-WP_002913892.1, NZ_KK737546.1, 1774891, 1776012, -, 1122, 877919; cds-WP_004149336.1, NZ_KK737546.1, 1776039, 1777034, -, 996, 1246544; cds-WP_002913894.1, NZ_KK737546.1, 1777311, 1778477, -, 1167, 786978; cds-WP_004144312.1, NZ_KK737546.1, 1778961, 1779392, -, 432, 2141976; cds-WP_004890414.1, NZ_KK737546.1, 1779553, 1781877, -, 2325, 37340; cds-WP_021440561.1, NZ_KK737546.1, 1781878, 1786827, -, 4950, 1114128; cds-WP_002913950.1, NZ_KK737546.1, 1787033, 1787890, +, 858, 345441; cds-WP_032429692.1, NZ_KK737546.1, 1788117, 1788959, +, 843, 77470; cds-WP_021440563.1, NZ_KK737546.1, 1789030, 1789806, -, 777, 479480; cds-WP_002913953.1, NZ_KK737546.1, 1789905, 1791191, -, 1287, 1154616; cds-WP_002913954.1, NZ_KK737546.1, 1791262, 1791462, -, 201, 555439; cds-WP_002913956.1, NZ_KK737546.1, 1791464, 1791799, -, 336, 677131; cds-WP_032429693.1, NZ_KK737546.1, 1791801, 1793651, -, 1851, 1665981; cds-WP_004174835.1, NZ_KK737546.1, 1793667, 1794182, -, 516, 864867; cds-WP_002913979.1, NZ_KK737546.1, 1794257, 1794580, -, 324, 526111; cds-WP_002913991.1, NZ_KK737546.1, 1794598, 1794984, -, 387, 440727; cds-WP_002913992.1, NZ_KK737546.1, 1795011, 1796225, -, 1215, 1656612; cds-WP_002913993.1, NZ_KK737546.1, 1796404, 1796895, -, 492, 466070; cds-WP_004185116.1, NZ_KK737546.1, 1797130, 1797864, -, 735, 262672; cds-WP_002913995.1, NZ_KK737546.1, 1797985, 1798788, +, 804, 440062; cds-WP_021440564.1, NZ_KK737546.1, 1798835, 1799671, -, 837, 7931; cds-WP_021440565.1, NZ_KK737546.1, 1799771, 1800751, -, 981, 28928; cds-WP_023301580.1, NZ_KK737546.1, 1800742, 1801380, -, 639, 27902; cds-WP_002914002.1, NZ_KK737546.1, 1801505, 1802782, +, 1278, 252359; cds-WP_004144329.1, NZ_KK737546.1, 1802779, 1803915, -, 1137, 145066; cds-WP_021440567.1, NZ_KK737546.1, 1803998, 1804831, -, 834, 15578; cds-WP_002914018.1, NZ_KK737546.1, 1804831, 1806267, -, 1437, 5117; cds-WP_015958809.1, NZ_KK737546.1, 1806337, 1809612, -, 3276, 25983; cds-WP_004214134.1, NZ_KK737546.1, 1809725, 1810921, +, 1197, 6888; cds-WP_002914024.1, NZ_KK737546.1, 1810995, 1811417, +, 423, 1383; cds-WP_002914027.1, NZ_KK737546.1, 1811473, 1812726, -, 1254, 7400237; cds-WP_004185137.1, NZ_KK737546.1, 1813052, 1814242, +, 1191, 185039; cds-WP_002914032.1, NZ_KK737546.1, 1814317, 1814655, -, 339, 178238; cds-WP_004149357.1, NZ_KK737546.1, 1814721, 1816058, -, 1338, 416716; cds-WP_002914033.1, NZ_KK737546.1, 1816045, 1816731, -, 687, 163313; cds-WP_004888415.1, NZ_KK737546.1, 1816761, 1818182, -, 1422, 146319; cds-WP_004185139.1, NZ_KK737546.1, 1818773, 1822660, -, 3888, 1011965; cds-WP_004144346.1, NZ_KK737546.1, 1822836, 1824452, +, 1617, 64332; cds-WP_071603195.1, NZ_KK737546.1, 1824449, 1824991, -, 543, 32744; cds-WP_004180922.1, NZ_KK737546.1, 1825021, 1825656, -, 636, 10014; cds-WP_004144349.1, NZ_KK737546.1, 1825870, 1826718, +, 849, 347660; cds-WP_004888420.1, NZ_KK737546.1, 1826775, 1827035, +, 261, 102322; cds-WP_004144351.1, NZ_KK737546.1, 1827048, 1827428, -, 381, 87553; cds-WP_004888422.1, NZ_KK737546.1, 1827428, 1828159, -, 732, 295750; cds-WP_002914062.1,
NZ_KK737546.1, 1828171, 1828908, -, 738, 438297; cds-WP_002914063.1, NZ_KK737546.1, 1828920, 1829825, -, 906, 2036644; cds-WP_002914065.1, NZ_KK737546.1, 1829822, 1830502, -, 681, 599061; cds-WP_002914067.1, NZ_KK737546.1, 1830752, 1831726, -, 975,1004806; cds-WP_002914069.1, NZ_KK737546.1, 1831742, 1833541, -, 1800, 3510419; cds-WP_004144354.1, NZ_KK737546.1, 1833727, 1834206, -, 480, 109916; cds-WP_029140701, NZ_KK737546.1, 1834203, 1835159, -, 957, 1684700; cds-WP_002914072.1, NZ_KK737546.1, 1835159, 1835809, -, 651, 2744542; cds-WP_002914074.1, NZ_KK737546.1, 1835842, 1836417, -, 576, 2128499; cds-WP_121980491.1, NZ_KK737546.1, 1836414, 1836509, -, 96, 37984; cds-WP_004144357.1, NZ_KK737546.1, 1836838, 1838457, +, 1620, 148519; cds-WP_002914079.1, NZ_KK737546.1, 1838442, 1839179, -, 738, 54254; cds-WP_002914082.1, NZ_KK737546.1, 1839311, 1840642, +, 1332, 1695291; cds-WP_002914084.1, NZ_KK737546.1, 1840688, 1841071, -, 384, 54144; cds-WP_002914088.1, NZ_KK737546.1, 1841384, 1842073, +, 690, 34340; cds-WP_002914089.1, NZ_KK737546.1, 1842131, 1843216, -, 1086, 39770; cds-WP_002914091.1, NZ_KK737546.1, 1843420, 1843845, +, 426, 193891; cds-WP_002914092.1, NZ_KK737546.1, 1843915, 1844613, +, 699, 37770; cds-WP_004149373.1, NZ_KK737546.1, 1844648, 1847299, +, 2652, 607237; cds-WP_002914094.1, NZ_KK737546.1, 1847420, 1848775, +, 1356, 501477; cds-WP_004888435.1, NZ_KK737546.1, 1848817, 1849140, +, 324, 11662; cds-WP_004144368.1, NZ_KK737546.1, 1849144, 1850442, -, 1299, 405638; cds-AF52_RS14425, NZ_KK737547.1, 98, 328, -, 231, 1972; cds-WP_002890376.1, NZ_KK737547.1, 349, 636, -, 288, 0; cds-AF52_RS0123765, NZ_KK737547.1, 642, 842, -, 201, 0; cds-AF52_RS27035, NZ_KK737547.1, 876, 1049, +, 174, 8249; cds-AF52_RS14415, NZ_KK737547.1, 4070, 4221, +, 152, 3753; cds-AF52_RS14410, NZ KK737547.1, 4277, 4528, -, 252, 106; cds-WP_002890386.1, NZ KK737547.1, 4875, 5477, -, 603, 943006; cds-AF52_RS14400, NZ KK737547.1, 5700, 5873, -, 174, 14306; cds-AF52_RS27040, NZ_KK737547.1, 7013, 7174, -, 162, 5211; cds-WP_032429763.1, NZ_KK737547.1, 7192, 7758, -, 567, 2455; cds-WP_032429764.1, NZ_KK737547.1, 7833, 7949, +, 117, 371; cds-WP_002890390.1, NZ KK737547.1, 8014, 9078, +, 1065, 547712; cds-WP_002890395.1, NZ_KK737547.1, 9187, 10314, +, 1128, 3318011; cds-WP_002890398.1, NZ_KK737547.1, 10337, 10669, +, 333, 2027704; cds-WP_0028904001, NZ_KK737547.1, 10696, 12543, +, 1848, 4132887; cds-WP_002890403.1, NZ_KK737547.1, 12554, 13525, +, 972, 501608; cds-WP_002890404.1, NZ_KK737547.1, 13662, 14027, +, 366, 22094; cds-WP_004144646.1, NZ_KK737547.1, 14052, 14747, +, 696, 13310; cds-WP_002890412.1, NZ KK737547.1, 14802, 15686, -, 885, 1865428; cds-WP_002890414.1, NZ_KK737547.1, 15985, 16524, -, 540, 82266; cds-WP_002890417.1, NZ_KK737547.1, 16672, 17121, +, 450, 56399; cds-WP_004144650.1, NZ_KK737547.1, 17125, 18228, +, 1104, 109188; cds-WP_001021161.1, NZ_KK737547.1, 18316,18786, +, 471, 510789; cds-WP_002891356.1, NZ _KK737547.1, 18806, 19225, +, 420, 1104461; cds-WP_004177267.1, NZ_KK737547.1, 19297, 20268, +, 972, 290796; cds-WP_004177266.1, NZ_KK737547.1, 20261, 20764, +, 504, 21052; cds-WP_004191727.1, NZ_KK737547.1, 20810, 21784, -, 975, 177519; cds-WP_004144655.1, NZ_KK737547.1, 21845, 23707, -, 1863, 628267; cds-WP_004144656.1, NZ_KK737547.1, 23726, 24625, -, 900, 344235; cds-WP_032429766.1, NZ_KK737547.1, 24626, 24868, -, 243, 72487; cds-WP_004151340.1, NZ_KK737547.1, 25050, 26498, +, 1449, 266996; cds-WP_032429768.1, NZ KK737547.1, 26565, 27041, -, 477, 39367; cds-WP_002891460.1, NZ_KK737547.1, 27092, 27685, -, 594, 15995; cds-WP_002891462.1, NZ_KK737547.1, 27648, 28559, -, 912, 75185; cds-WP_002891465.1, NZ_KK737547.1, 28751, 29242, +, 492, 437824; cds-WP_004144660.1, NZ_KK737547.1, 29286,30650, -, 1365, 162323; cds-WP_004177261.1, NZ_KK737547.1, 30810, 31988, -, 1179, 167345; cds-WP_002891473.1, NZ_KK737547.1, 32320, 33684, -, 1365, 990; cds-WP_002891476.1, NZ KK737547.1, 33695, 34513, -, 819, 7661; cds-WP_032103307.1, NZ_KK737547.1, 34588, 36267, -, 1680, 1642; cds-WP_002891541.1, NZ_KK737547.1, 36278, 36598, -, 321, 20; cds-WP_032429771.1, NZ KK737547.1, 36612, 38288, -, 1677, 1407; cds-WP_032419345.1, NZ KK737547.1, 38285, 39106, -, 822, 3705; cds-WP_002891624.1, NZ_KK737547.1, 39259, 40017, +, 759, 57657; cds-WP_004178748.1, NZ_KK737547.1, 40057, 40389, -, 333, 66937; cds-WP_002891629.1, NZ_KK737547.1, 40639, 41526, -, 888, 3958484; cds-WP_002891634.1, NZ_KK737547.1, 41538, 41867, -, 330, 2163146; cds-WP_002891635.1, NZ_KK737547.1, 41867, 42478, -, 612, 6154700; cds-WP 032424651.1, NZ_KK737547.1, 42468, 44459, -, 1992, 20664858; cds-WP 002891641.1, NZ_KK737547.1, 44479, 45423, -, 945, 12372381; cds-WP_032429776.1, NZ KK737547.1, 45903, 47378, -, 1476, 102547; cds-WP_002891647.1, NZ_KK737547.1, 47422, 48000, -, 579, 265209; cds-WP_032429779.1, NZ_KK737547.1, 48303, 48620, +, 318, 83877; cds-WP_004147333.1, NZ_KK737547.1, 48966, 50264, +, 1299, 8571044; cds-WP_002891804.1, NZ KK737547.1, 50574, 51197, +, 624, 1780159; cds-WP_002891807.1, NZ_KK737547.1, 51448, 52722, +, 1275, 1859620; cds-WP_004151336.1, NZ_KK737547.1, 52906, 55260, +, 2355, 4555732; cds-WP_002444653.1, NZ KK737547.1, 55470, 55742, +, 273, 1354090; cds-WP_004177252.1, NZ_KK737547.1, 55957, 57831, +, 1875, 1018832; cds-WP_004144680.1, NZ KK737547.1, 57980, 58348, +, 369, 7422; cds-WP 002891860.1, NZ_KK737547.1, 58460, 58861, +, 402, 49972; cds-WP_002891863.1, NZ_KK737547.1, 58973, 59674, -, 702, 111838; cds-WP_032429782.1, NZ_KK737547.1, 59741, 61441, -, 1701, 61314; cds-WP_004151335.1, NZ_KK737547.1, 61569, 62387, +, 819, 47454; cds-WP_002891871.1, NZ KK737547.1, 62432, 63484, -, 1053, 91569; cds-WP_002891873.1, NZ_KK737547.1, 63600, 64058, +, 459, 59440; cds-WP_032429784.1, NZ_KK737547.1, 64102, 65874, +, 1773, 281123; cds-WP_032429785.1, NZ KK737547.1, 65867, 67645, +, 1779, 68565; cds-WP_002891893.1, NZ KK737547.1, 67889, 68227, +, 339, 85; cds-WP_004152876.1, NZ KK737547.1, 68261, 69547, +, 1287, 20500; cds-WP_020804789.1, NZ KK737547.1, 69602, 70465, -, 864, 117982; cds-WP_002891903.1, NZ_KK737547.1, 70682, 71173, +, 492, 86484; cds-WP_004142687.1, NZ_KK737547.1, 71211, 71522, -, 312, 11108; cds-WP_004178755.1, NZ_KK737547.1, 71590, 71997, -, 408, 80318; cds-WP_013815099.1-31, NZ KK737547.1, 72608, 73576, -, 969, 126563; cds-WP_004147373.1, NZ_KK737547.1, 74028, 77174, -, 3147, 5463951; cds-WP_004177236.1, NZ KK737547.1, 77197, 78390, -, 1194, 2821536; cds-WP_002892080.1, NZ_KK737547.1, 78533, 79183, +, 651, 222061; cds-WP_032429786.1, NZ_KK737547.1, 79313, 82660, +, 3348, 1456054; cds-WP_032429788.1, NZ KK737547.1, 82696, 83595, -, 900, 35929; cds-WP_002892136.1, NZ_KK737547.1, 83663, 83836, -, 174, 14896; cds-WP_002892142.1, NZ_KK737547.1, 83849, 84376, -, 528, 5852; cds-WP_002892144.1, NZ_KK737547.1, 84446, 84823, +, 378, 7742; cds-WP_002892145.1, NZ_KK737547.1, 84974, 85525, +, 552, 276003; cds-WP_032429789.1, NZ_KK737547.1, 85618, 87525, +, 1908, 301165; cds-WP_002892173.1, NZ_KK737547.1, 87583, 87915, +, 333, 175413; cds-WP_002892177.1, NZ_KK737547.1, 87915, 88520, +, 606, 268229; cds-WP_002892181.1, NZ KK737547.1, 88632, 90506, +, 1875, 2627528; cds-WP_032423667.1, NZ KK737547.1, 90729, 91373, +, 645, 401190; cds-WP_002892189.1, NZ_KK737547.1, 91502, 92464, +, 963, 73258; cds-WP_002892192.1, NZ KK737547.1, 92528, 93832, +, 1305, 191639; cds-WP_002892195.1, NZ KK737547.1, 93868, 95544, -, 1677, 194975; cds-WP_004147385.1, NZ_KK737547.1, 95770, 96990, -, 1221, 2887; cds-WP_002892203.1, NZ_KK737547.1, 97260, 98912, +, 1653, 289483; cds-WP_002892205.1, NZ_KK737547.1, 99018, 99497, -, 480, 47726; cds-WP_004151327.1, NZ_KK737547.1, 99700, 100494, -, 795, 26260; cds-WP_004893800.1, NZ KK737547.1, 100626, 103127, -, 2502, 180177; cds-WP_002892208.1, NZ_KK737547.1, 103234, 103644, +, 411, 13163; cds-WP_004893797.1, NZ KK737547.1, 103641, 104099, -, 459, 24645; cds-WP_002892258.1, NZ_KK737547.1, 104096, 105013, -, 918, 106223; cds-WP_004893793.1, NZ_KK737547.1, 105154, 105831, +, 678, 30641; cds-WP_004893788.1, NZ_KK737547.1, 105818, 106600, +, 783, 136969; cds-WP_002892263.1, NZ_KK737547.1, 106708, 107562, -, 855, 457456; cds-WP_009484702.1, NZ_KK737547.1, 107621, 108430, -, 810, 230047; cds-WP_004196998.1, NZ_KK737547.1, 108420, 109043, -, 624, 37536; cds-WP_032429794.1, NZ_KK737547.1, 109014, 109700, +, 687, 21131; cds-WP_032429796.1, NZ_KK737547.1, 109697, 112111, +, 2415, 84430; cds-WP_004219504.1, NZ_KK737547.1, 112088, 112201, -, 114, 650; cds-WP_004142997.1, NZ_KK737547.1, 112293, 113438, +, 1146, 14824; cds-WP_004151323.1, NZ_KK737547.1, 113546, 114622, -, 1077, 51527; cds-WP_032429798.1, NZ_KK737547.1, 114734, 115801, -, 1068, 92684; cds-WP_004151322.1, NZ_KK737547.1, 115798, 116307, -, 510, 63144; cds-WP_032429801.1, NZ_KK737547.1, 116404, 116742, -, 339, 33724; cds-WP_004151320.1, NZ KK737547.1, 116850, 117572, -, 723, 417009; cds-WP_004151319.1, NZ_KK737547.1, 117576, 118070, -, 495, 380935; cds-WP_004143010.1, NZ_KK737547.1, 118246, 119631, +, 1386, 792501; cds-WP_004143016.1, NZ_KK737547.1, 119677, 119889, -, 213, 32605; cds-WP_004143017.1, NZ KK737547.1, 119891, 120757, -, 867, 363687; cds-WP_013815099.1-32, NZ_KK737547.1, 121337, 122305, +, 969, 118264; cds-AF52_RS25480, NZ_KK737547.1, 122362, 122511, +, 150, 927; cds-WP_002893005.1, NZ KK737547.1, 122529, 123590, +, 1062, 1898; cds-AF52_RS13845, NZ_KK737547.1, 123655, 124524, +, 870, 9556; cds-WP_013815099.1-33, NZ KK737547.1, 124555, 125523, +, 969, 112625; cds-AF52_RS27075, NZ KK737547.1, 125582, 125707, +, 126, 4800; cds-AF52_RS13835, NZ_KK737547.1, 125760, 126170, -, 411, 52; cds-AF52_RS27080, NZ_KK737547.1, 127801, 127981, -, 181, 5461; cds-AF52_RS13830, NZ_KK737547.1, 128015, 130339, -, 2325, 167471; cds-WP_021441375.1, NZ_KK737547.1, 130436, 131470, -, 1035, 29857; cds-WP_004151823.1, NZ_KK737547.1, 132136, 133002, -, 867, 63081; cds-WP_004223482.1, NZ _KK737547.1, 133111, 134538, +, 1428,461; cds-WP_004142570.1, NZ_KK737547.1, 134609, 135853, +, 1245, 127477; cds-WP_004176981.1, NZ_KK737547.1, 135857, 136837, -, 981, 90028; cds-WP_004183289.1, NZ KK737547.1, 137086, 137502, +, 417, 9981; cds-WP_016946855.1, NZ_KK737547.1, 137522, 138673, -, 1152, 1473; cds-WP_032429809.1, NZ KK737547.1, 138675, 139697, -, 1023, 791; cds-WP_021441372.1, NZ_KK737547.1, 139714, 140919, -, 1206, 1821; cds-WP_021441371.1, NZ_KK737547.1, 141107, 142483, -, 1377, 29313; cds-WP_032428635.1, NZ_KK737547.1, 142591, 144789, -, 2199, 60749; cds-WP_032428633.1, NZ KK737547.1, 144849, 145931, -, 1083, 257; cds-WP_032428631.1, NZ_KK737547.1, 146327, 147811, -, 1485, 103382; cds-WP_004176990.1, NZ_KK737547.1, 147970, 149271, +, 1302, 5587; cds-WP_002892824.1, NZ_KK737547.1, 149285, 149650, +, 366, 44; cds-WP_021441367.1, NZ KK737547.1, 150030, 151529, +, 1500, 14366; cds-WP_021441366.1, NZ_KK737547.1, 151507, 152049, +, 543, 41800; cds-WP_004178885.1, NZ_KK737547.1, 152067, 152429, -, 363, 72645; cds-WP_004178884.1, NZ_KK737547.1, 152430, 152732, -, 303,163604; cds-WP_021441365.1, NZ KK737547.1, 152816, 153931, -, 1116, 115126; cds-WP_004183273.1, NZ_KK737547.1, 153948, 155120, -, 1173, 55679; cds-WP_002892774.1, NZ_KK737547.1, 155238, 155921, -, 684, 422814; cds-WP_009308086.1, NZ_KK737547.1, 156346, 157533, +, 1188, 181; cds-WP_004223434.1, NZ_KK737547.1, 157578, 159311, +, 1734, 61324; cds-WP_004183270.1, NZ_KK737547.1, 159399, 160175, +, 777, 1; cds-WP_032429971.1, NZ_KK737547.1, 160201, 160935, +, 735, 20386; cds-WP_013815099.1-34, NZ_KK737547.1, 160961, 161929, -, 969, 89015; cds-AF52_RS13690, NZ_KK737547.1, 161961, 162431, -, 471, 8; cds-WP_021441356.1, NZ KK737547.1, 162670, 163593, +, 924, 0; cds-WP_004147437.1, NZ KK737547.1, 163758, 165275, +, 1518, 0; cds-WP_021441355.1, NZ_KK737547.1, 165380, 166750, +, 1371, 0; cds-WP_021441354.1, NZ_KK737547.1, 166750, 168150, +, 1401, 0; cds-WP_187079191.1, NZ_KK737547.1, 168174, 169184, +, 1011, 0; cds-WP_004177009.1, NZ KK737547.1, 169200, 169841, -, 642, 0; cds-WP_004152946.1, NZ_KK737547.1, 170025, 171047, -, 1023, 0; cds-WP_004142660.1, NZ_KK737547.1, 171067, 172551, -, 1485, 0; cds-WP_002892597.1, NZ_KK737547.1, 172584, 173507, -, 924, 0; cds-WP_004191476.1, NZ_KK737547.1, 173937, 174956, -, 1020, 0; cds-AF52_RS13635, NZ_KK737547.1, 175058, 176062, +, 1005, 0; cds-WP_004147428.1, NZ_KK737547.1, 176984, 178015, -, 1032, 0; cds-WP_004191478.1, NZ_KK737547.1, 178025, 179197, -, 1173, 0; cds-WP_004215104.1, NZ_KK737547.1,
179194, 180114, -, 921, 0; cds-WP_187079192.1, NZ_KK737547.1, 180220, 181188, -, 969, 96885; cds-AF52_RS0123810, NZ_KK737547.1, 181222, 181695, +, 474, 39233; cds-WP_032429814.1, NZ_KK737547.1, 181760, 183334, -, 1575, 67862; cds-WP_002892402.1, NZ_KK737547.1, 183628, 183900, -, 273, 219389; cds-WP_002892400.1, NZ_KK737547.1, 184001, 184921, -, 921, 180254; cds-WP_032429816.1, NZ_KK737547.1, 185432, 186298, +, 867, 80798; cds-WP 032428620.1, NZ_KK737547.1, 186371, 187675, -, 1305, 3861; cds-WP_004142684.1, NZ_KK737547.1, 187721, 187861, -, 141, 0; cds-WP_002892370.1, NZ_KK737547.1, 188186, 188356, +, 171, 19207; cds-AF52_RS27950, NZ_KK737547.1, 188435, 188512, -, 78, 8504; cds-WP_013815099.1-35, NZ_KK737547.1, 188546, 189514, +, 969, 107322; cds-AF52_RS13575, NZ_KK737547.1, 189571, 190284, -, 714, 238; cds-WP_004183295.1, NZ_KK737547.1, 190287, 191030, -, 744, 659; cds-WP_004176974.1, NZ_KK737547.1, 191045, 191515, -, 471, 98; cds-WP_002893017.1, NZ_KK737547.1, 191508, 191942, -, 435, 32; cds-WP_004178896.1, NZ_KK737547.1, 192190, 192843, -, 654, 169857; cds-WP_004142557.1, NZ_KK737547.1, 192963, 193331, -, 369, 21712; cds-WP_014599061.1, NZ_KK737547.1, 193331, 193912, -, 582, 59410; cds-WP_171991789.1, NZ_KK737547.1, 194092, 195207, +, 1116, 345369; cds-WP_002893035.1, NZ_KK737547.1, 195238, 195579, +, 342, 194085; cds-WP_002893037.1, NZ_KK737547.1, 195597, 195845, -, 249, 78641; cds-WP_032428645.1, NZ_KK737547.1, 196348, 199071, +, 2724, 218080; cds-WP_021441380.1, NZ_KK737547.1, 199124, 200203, +, 1080,323; cds-WP_032429823.1, NZ_KK737547.1, 200217, 203309, +, 3093, 11165; cds-WP_004176956.1, NZ_KK737547.1, 203658, 204377, -, 720, 12902; cds-WP_004178906.1, NZ_KK737547.1, 204425, 205540, -, 1116, 1610; cds-WP_021441381.1, NZ_KK737547.1, 205867, 207534, +, 1668, 79781; cds-WP_004178910.1, NZ_KK737547.1, 207553, 208506, +, 954, 0; cds-WP_008805546.1, NZ_KK737547.1, 208509, 209402, +, 894, 0; cds-WP_004223507.1, NZ_KK737547.1, 209406, 210443, +, 1038, 16; cds-WP_021441382.1, NZ_KK737547.1, 210433, 211443, +, 1011, 3445; cds-WP_021441383.1, NZ_KK737547.1, 211541, 212809, +, 1269, 38837; cds-WP_032428650.1, NZ_KK737547.1, 212913, 213821, -, 909, 75; cds-WP_032429824.1, NZ_KK737547.1, 213946, 215280, +, 1335, 6397; cds-WP_004176948.1, NZ_KK737547.1, 215277, 216740, -, 1464, 117; cds-WP_004176946.1, NZ_KK737547.1, 216982, 217833, +, 852, 0; cds-WP_002893187.1, NZ_KK737547.1, 217881, 218522, +, 642, 0; cds-WP_002893189.1, NZ_KK737547.1, 218537, 219202, +, 666, 1174; cds-WP_004151676.1, NZ_KK737547.1, 219195, 219956, +, 762, 0; cds-WP_032428652.1, NZ_KK737547.1, 220010, 220774, -, 765, 40; cds-WP_004183313.1, NZ_KK737547.1, 220955, 222292, +, 1338, 0; cds-WP_104443363.1, NZ_KK737547.1, 222667, 222822, -, 156, 6; cds-WP_077254746.1, NZ_KK737547.1, 223649, 223759, -, 111, 3011; cds-WP_013815099.1-36, NZ_KK737547.1, 223803, 224771, -, 969, 84879; cds-WP_022615459.1, NZ_KK737547.1, 225071, 226066, -, 996, 58006; cds-WP_009483809.1, NZ_KK737547.1, 226423, 227127, -, 705, 12728; cds-WP_021441389.1, NZ_KK737547.1, 227114, 227950, -, 837, 3211; cds-WP_032429827.1, NZ_KK737547.1, 227943, 228776, -, 834, 16681; cds-WP_021441390.1, NZ_KK737547.1, 228776, 229798, -, 1023, 12174; cds-WP_009308119.1, NZ_KK737547.1, 229918, 231486, -, 1569, 96673; cds-WP_002893466.1, NZ_KK737547.1, 231692, 232387, +, 696, 15612; cds-WP_032429829.1, NZ_KK737547.1, 232508, 234172, -, 1665, 303764; cds-WP_004147503.1, NZ_KK737547.1, 234186, 235658, -, 1473, 541257; cds-WP_004151671.1, NZ_KK737547.1, 235672, 236259, -, 588, 22169; cds-WP_004142478.1, NZ_KK737547.1, 236388, 238421, +, 2034, 27997; cds-WP_071603220.1, NZ_KK737547.1, 238467, 238994, -, 528, 102977; cds-WP_021441392.1, NZ_KK737547.1, 239138, 240394, +, 1257, 58155; cds-WP_021441393.1, NZ_KK737547.1, 240391, 241284, -, 894, 7289; cds-WP_004183326.1, NZ_KK737547.1, 241539, 242144, +, 606, 28603; cds-WP_021441395.1, NZ_KK737547.1, 242203, 243702, -, 1500, 5481096; cds-WP_032428654.1, NZ_KK737547.1, 243717, 245135, -, 1419, 9418081; cds-WP_004893550.1, NZ_KK737547.1, 245170, 246120, -, 951, 6265779; cds-WP_004893549.1, NZ_KK737547.1, 246113, 246943, -, 831, 6587574; cds-WP_032429976.1, NZ_KK737547.1, 247255, 248205, +, 951, 476712; cds-WP_021441398.1, NZ_KK737547.1, 248202, 249164, -, 963, 438720; cds-WP_004885708.1, NZ_KK737547.1, 249320, 250408, -, 1089, 4576918; cds-WP_032429833.1, NZ_KK737547.1, 250410, 251936, -, 1527, 11740788; cds-WP_032428655.1, NZ_KK737547.1, 251936, 252544, -, 609, 4899624; cds-WP_021441401.1, NZ_KK737547.1, 252555, 253496, -, 942, 5522241; cds-WP_021441402.1, NZ_KK737547.1, 253614, 254273, -, 660, 66600; cds-WP_021441403.1, NZ_KK737547.1, 254340, 256568, -, 2229, 795972; cds-WP_004147519.1, NZ_KK737547.1, 256828, 258036, +, 1209, 25053; cds-WP_004176919.1, NZ_KK737547.1, 258064, 258267, +, 204, 1758; cds-WP_021441404.1, NZ_KK737547.1, 258278, 262159, +, 3882, 274012; cds-WP_002893737.1, NZ_KK737547.1, 262224, 263018, -, 795, 40816; cds-WP_004147521.1, NZ_KK737547.1, 263015, 264007, -, 993, 1532; cds-WP_032429835.1, NZ_KK737547.1, 264004, 265011, -, 1008, 93208; cds-WP_032429836.1, NZ_KK737547.1, 265124, 266365, +, 1242, 10123; cds-WP_004147526.1, NZ_KK737547.1, 266628, 267587, -, 960, 94477; cds-WP_032429837.1, NZ_KK737547.1, 267776, 268951, +, 1176, 52608; cds-WP_004147529.1, NZ_KK737547.1, 268961, 270568, +, 1608, 51446; cds-WP_020324582.1, NZ_KK737547.1, 270582, 271433, +, 852, 285072; cds-WP_024622880.1, NZ_KK737547.1, 271442, 272188, +, 747, 68530; cds-WP_002893892.1, NZ_KK737547.1, 272189, 272602, +, 414, 9998; cds-WP_021441407.1, NZ_KK737547.1, 272959, 275064, +, 2106, 1074299; cds-WP_002893903.1, NZ_KK737547.1, 275169, 275366, +, 198, 119357; cds-WP_004178982.1, NZ_KK737547.1, 275363, 275767, -, 405, 141853; cds-WP_021441408.1, NZ_KK737547.1, 275742, 276044, -, 303, 13347; cds-WP_021464368.1, NZ_KK737547.1, 276228, 276971, -, 744, 2038; cds-WP_032429838.1, NZ_KK737547.1, 277029, 278117, -, 1089, 0; cds-WP_020801584.1, NZ_KK737547.1, 278281, 279783, +, 1503, 76; cds-WP_002894035.1, NZ_KK737547.1, 279780, 280778, +, 999, 2170; cds-WP_002894036.1, NZ_KK737547.1, 280799, 281863, +, 1065, 9724; cds-WP_004178989.1, NZ_KK737547.1, 281965, 283206, +, 1242, 168287; cds-WP_004210859.1, NZ_KK737547.1, 283420, 284619, -, 1200, 364; cds-WP_004183373.1, NZ_KK737547.1, 284721, 285749, +, 1029, 3335; cds-WP_032429839.1, NZ_KK737547.1, 285810, 287078, -, 1269, 78548; cds-WP_032429841.1, NZ_KK737547.1, 287104, 288462, -, 1359, 22657; cds-WP_004151655.1, NZ_KK737547.1, 288573, 289382, -, 810, 183754; cds-WP_072157843.1, NZ_KK737547.1, 289504, 290454, -, 951, 6262; cds-WP_032429842.1, NZ_KK737547.1, 290600, 291838, -, 1239, 0; cds-WP_002894159.1, NZ_KK737547.1, 291835, 293070, -, 1236, 0; cds-WP_032429843.1, NZ_KK737547.1, 293067, 294329, -, 1263, 41648; cds-WP_002894164.1, NZ_KK737547.1, 294781, 295389, +, 609, 2022; cds-WP_032429844.1, NZ_KK737547.1, 295402, 296898, +, 1497, 1763; cds-WP_032429847.1, NZ_KK737547.1, 296895, 297923, +, 1029, 174; cds-WP_032429849.1, NZ_KK737547.1, 297966, 298907, -, 942, 2180; cds-WP_004223619.1, NZ_KK737547.1, 298919, 299923, -, 1005, 0; cds-WP_004147555.1, NZ_KK737547.1, 299920, 300909, -, 990, 0; cds-WP_021441413.1, NZ_KK737547.1, 300906, 302408, -, 1503, 946; cds-WP_032429851.1, NZ_KK737547.1, 302453, 303433, -, 981, 615; cds-WP_032429853.1, NZ_KK737547.1, 303842, 306151, +, 2310, 0; cds-WP_002894357.1, NZ_KK737547.1, 306259, 306801, -, 543, 12225; cds-WP_004142397.1, NZ_KK737547.1, 306798, 307487, -, 690, 38935; cds-WP_020802207.1, NZ_KK737547.1, 307629, 308774, +, 1146, 3; cds-WP_004212613.1, NZ_KK737547.1, 308775, 309401, -, 627, 47384; cds-WP_002894369.1, NZ_KK737547.1, 309386, 310609, -, 1224, 79364; cds-WP_004147565.1, NZ_KK737547.1, 310720, 311652, -, 933, 39881; cds-WP_002894375.1, NZ_KK737547.1, 311833, 312582, -, 750, 6292; cds-WP_002894394.1, NZ_KK737547.1, 312994, 313557, +, 564, 3177344; cds-WP_004183398.1, NZ_KK737547.1, 313745, 315310, +, 1566, 1004454; cds-WP_002894401.1, NZ_KK737547.1, 315387, 315815, -, 429, 45962; cds-WP_002894404.1, NZ_KK737547.1, 316002, 316412, -, 411, 142584; cds-WP_032429854.1, NZ_KK737547.1, 316591, 317388, -, 798, 33598; cds-WP_004142371.1, NZ_KK737547.1, 317481, 318854, -, 1374, 129290; cds-WP_072143258.1, NZ_KK737547.1, 319136, 319711, +, 576, 215433; cds-WP_002439184.1, NZ_KK737547.1, 319906, 320115, +, 210, 1411471; cds-WP_002894459.1, NZ_KK737547.1, 320183, 320566, -, 384, 109315; cds-WP_002894461.1, NZ_KK737547.1, 320656, 321444, +, 789, 96487; cds-WP_002894464.1, NZ_KK737547.1, 321687, 321893, +, 207, 47421; cds-WP_002894467.1, NZ_KK737547.1, 322084, 323049, -, 966, 1807706; cds-WP_020803604.1, NZ_KK737547.1, 323230, 324189, -, 960, 106567; cds-WP_002894472.1, NZ_KK737547.1, 324320, 324964, -, 645, 96620; cds-WP_002894474.1, NZ_KK737547.1, 325069, 325332, -, 264, 105601; cds-WP_002894539.1, NZ_KK737547.1, 325445, 326644, -, 1200, 964851; cds-WP_004147579.1, NZ_KK737547.1, 326783, 327931, -, 1149, 626250; cds-WP_002894613.1, NZ_KK737547.1, 327943, 329055, -, 1113, 125684; cds-WP_002894617.1, NZ_KK737547.1, 329055, 330956, -, 1902, 541301; cds-WP_002894620.1, NZ_KK737547.1, 330984, 331451, -, 468, 253214; cds-WP_002894623.1, NZ_KK737547.1, 331455, 331772, -, 318, 120045; cds-WP_004151650.1, NZ_KK737547.1, 331995, 332624, -, 630, 14421; cds-WP_004176879.1, NZ_KK737547.1, 332697, 333347, -, 651, 258468; cds-WP_032429857.1, NZ_KK737547.1, 333340, 334371, -, 1032, 473285; cds-WP_002894693.1, NZ_KK737547.1, 334371, 334961, -, 591, 596226; cds-WP_004147581.1, NZ_KK737547.1, 334976, 337558, -, 2583, 3344331; cds-WP_032429977.1, NZ_KK737547.1, 337785, 338267, +, 483, 57647; cds-WP_032429859.1, NZ_KK737547.1, 338313, 340106, -, 1794, 46679; cds-WP_032429860.1, NZ_KK737547.1, 340172, 341842, -, 1671, 5999; cds-WP_002894706.1, NZ_KK737547.1, 342216, 342941, -, 726, 290778; cds-WP_002894707.1, NZ_KK737547.1, 342941, 343615, -, 675, 266857; cds-WP_002894709.1, NZ_KK737547.1, 343615, 344355, -, 741, 228136; cds-WP_002894712.1, NZ_KK737547.1, 344520, 345431, -, 912, 1511799; cds-WP_032429861.1, NZ_KK737547.1, 345786, 347324, -, 1539, 659377; cds-WP_004197606.1, NZ_KK737547.1, 347450, 348328, -, 879, 1232034; cds-WP_002894722.1, NZ_KK737547.1, 348476, 348949, -, 474, 439781; cds-WP_004176871.1, NZ_KK737547.1, 348946, 349992, -, 1047, 368834; cds-WP_002894730.1, NZ_KK737547.1, 350146, 351570, -, 1425, 1445594; cds-WP_032429863.1, NZ_KK737547.1, 351750, 352925, +, 1176, 23577; cds-WP_002894734.1, NZ_KK737547.1, 354033, 355697, -, 1665, 104408; cds-WP_002894738.1, NZ_KK737547.1, 355976, 356728, -, 753, 132508; cds-WP_002894742.1, NZ_KK737547.1, 356845, 358065, -, 1221, 808857; cds-WP_002894747.1, NZ_KK737547.1, 358074, 359222, -, 1149, 550694; cds-WP _002894749.1, NZ_KK737547.1, 359336, 360136, -, 801, 993594; cds-WP_032429865.1, NZ_KK737547.1, 360451, 362418, +, 1968, 797303; cds-WP_002894753.1, NZ_KK737547.1, 362597, 364264, +, 1668, 2855847; cds-WP_004142270.1, NZ_KK737547.1, 364318, 364626, -, 309, 4329; cds-WP_004152230.1, NZ_KK737547.1, 364699, 366102, +, 1404, 31599; cds-WP_002894759.1, NZ_KK737547.1, 366150, 366482, +, 333, 5129; cds-WP_166686314.1, NZ_KK737547.1, 366534, 366977, -, 444, 56948; cds-WP_002894761.1, NZ_KK737547.1, 367099, 367551, -, 453, 955694; cds-WP_002894765.1, NZ_KK737547.1, 367843, 368373, -, 531, 381317; cds-WP_002894767.1, NZ_KK737547.1, 368525, 368818, -, 294, 86266; cds-WP_004152229.1, NZ_KK737547.1, 368952, 369725, -, 774, 157401; cds-WP_002894771.1, NZ_KK737547.1, 369909, 370457, +, 549, 1066784; cds-WP_002894773.1, NZ_KK737547.1, 370490, 372130, +, 1641, 3674512; cds-WP_004179052.1, NZ_KK737547.1, 372185, 372622, +, 438, 219332; cds-WP_002894776.1, NZ_KK737547.1, 372604, 373281, -, 678, 147672; cds-WP_021441429.1, NZ_KK737547.1, 373278, 375965, -, 2688, 24059; cds-WP_032429868.1, NZ_KK737547.1, 375966, 376541, -, 576, 0; cds-WP_021441430.1, NZ_KK737547.1, 376552, 378600, -, 2049, 18; cds-WP_004152226.1, NZ_KK737547.1, 378621, 380300, -, 1680, 593; cds-WP_020323459.1, NZ_KK737547.1, 380300, 380389, -, 90, 0; cds-WP_004142243.1, NZ_KK737547.1, 380696, 380905, +, 210, 850; cds-WP_032429869.1, NZ_KK737547.1, 381089, 382531, +, 1443, 37365; cds-WP_004893379.1, NZ_KK737547.1, 382503, 383981, -, 1479, 10909; cds-WP_002894919.1, NZ_KK737547.1, 384249, 384992, +, 744, 16210; cds-WP_004176857.1,
NZ_KK7375471, 385007, 385663, +, 657,66059; cds-WP_004183433.1, NZ_KK737547.1, 385657, 386589, +, 933,799927; cds-WP_004183435.1, NZ_KK737547.1,386579, 387322, +, 744, 43630; cds-WP_002894928.1, NZ_KK737547.1, 387404, 388129, +, 726, 48084; cds-WP_004176855.1, NZ_KK737547.1, 388126, 389121, +, 996, 17400; cds-WP_004183438.1, NZ_KK737547.1, 389131, 389775, +, 645, 17329; cds-WP_002894935.1, NZ_KK737547.1, 389791, 390582, +, 792, 120566; cds-WP_004183439.1, NZ_KK737547.1, 390673, 391956, -, 1284, 12944352; cds-WP_004142216.1, NZ_KK737547.1, 392597, 393001, +, 405, 5320365; cds-WP_002894949.1, NZ_KK737547.1, 392995, 393342, +, 348, 6047984; cds-WP_004179067.1, NZ_KK737547.1, 393342, 395108, +, 1767, 18938181; cds-WP_002894954.1, NZ_KK737547.1, 395124, 395840, +, 717, 5803723; cds-WP_012068464.1, NZ_KK737547.1, 396222, 399029, +, 2808, 27024889; cds-WP_004142209.1, NZ_KK737547.1, 399044, 400270, +, 1227, 6268512; cds-WP_002895039.1, NZ_KK737547.1, 400374, 401540, +, 1167, 8673088; cds-WP_002895043.1, NZ_KK737547.1, 401540, 402409, +, 870, 3387838; cds-WP_002895047.1, NZ_KK737547.1, 403234, 404802, +, 1569, 2416197; cds-WP_002895049.1, NZ_KK737547.1, 404818, 405957, +, 1140, 1366863; cds-WP_002895051.1, NZ_KK737547.1, 405973, 406089, +, 117, 99743; cds-WP_002895053.1, NZ_KK737547.1, 406089, 406379, +, 291, 29736; cds-WP_002895056.1, NZ_KK737547.1, 406529, 406933, +, 405, 436989; cds-WP_004152221.1, NZ_KK737547.1, 406930, 407622, +, 693, 1202036; cds-WP_002895061.1, NZ_KK737547.1, 407626, 408054, +, 429, 325615; cds-WP_004179068.1, NZ_KK737547.1, 408154, 409479, +, 1326, 850591; cds-WP_032429872.1, NZ_KK737547.1, 409609, 410901, +, 1293, 2741269; cds-WP_002895068.1, NZ_KK737547.1, 410936, 411460, +, 525, 3120660; cds-WP_002895071.1, NZ_KK737547.1, 411470, 412264, +, 795, 1820126; cds-WP_032409073.1, NZ_KK737547.1, 413744, 413893, -, 150, 1815; cds-WP_002895074.1, NZ_KK737547.1, 413963, 415006, +, 1044,30470; cds-WP_004151689.1, NZ_KK737547.1, 415042, 415761, +, 720, 54509; cds-WP_004147641.1, NZ_KK737547.1, 415758, 416702, -, 945, 10267; cds-WP_002895084.1, NZ_KK737547.1, 416820, 417185, -, 366, 91257; cds-WP_002895086.1, NZ_KK737547.1, 417500, 418552, +, 1053, 12692; cds-WP_002895089.1, NZ_KK737547.1, 418626, 419378, -, 753, 5333687; cds-WP_023302155.1, NZ_KK737547.1, 419670, 420713, -, 1044, 517873; cds-WP_004179071.1, NZ_KK737547.1, 420707, 421855, -, 1149, 818264; cds-WP_004142166.1, NZ_KK737547.1, 421858, 422904, -, 1047, 1986555; cds-WP_002895150.1, NZ_KK737547.1, 422914, 423930, -, 1017, 1402109; cds-WP_032428718.1, NZ_KK737547.1, 424140, 425612, -, 1473, 78046; cds-WP_032429873.1, NZ_KK737547.1, 425680, 426468, -, 789, 21176; cds-WP_002895156.1, NZ_KK737547.1, 426621, 426770, +, 150, 3911; cds-WP_032428721.1, NZ_KK737547.1, 426913, 427686, +, 774, 140771; cds-WP_002895159.1, NZ_KK737547.1, 427686, 428375, +, 690, 57819; cds-WP_004176832.1, NZ_KK737547.1, 428378, 429436, +, 1059, 193198; cds-WP_002895164.1, NZ_KK737547.1, 429437, 430255, -, 819, 64049; cds-WP_002895166.1, NZ_KK737547.1, 430484, 431479, +, 996, 431903; cds-WP_002895168.1, NZ_KK737547.1, 431589, 431855, -, 267, 681374; cds-WP_004147654.1, NZ_KK737547.1, 431907, 432212, -, 306, 534032; cds-WP_004151693.1, NZ_KK737547.1, 432339, 432584, -, 246, 317490; cds-WP_004176831.1, NZ_KK737547.1, 432913, 434124, +, 1212, 261455; cds-WP_032429874.1, NZ_KK737547.1, 434280, 435014, +, 735, 465946; cds-WP_004151695.1, NZ_KK737547.1, 435056, 435610, -, 555, 169933; cds-WP_002895280.1, NZ_KK737547.1, 435692, 435904, -, 213, 26331; cds-WP_032428722.1, NZ_KK737547.1, 436289, 436435, +, 147, 44; cds-WP_002895283.1, NZ_KK737547.1, 436640, 437146, +, 507,12223; cds-WP_072271782.1, NZ_KK737547.1, 437163, 438086, -, 924,34578; cds-AF52_RS0123875, NZ_KK737547.1, 438125, 441190, -, 3066, 26654; cds-WP_032429876.1, NZ_KK737547.1, 441191, 442276, -, 1086, 0; cds-WP_021441450.1, NZ_KK737547.1, 442810, 443241, +, 432, 3809; cds-WP_020316497.1, NZ_KK737547.1, 443293, 445980, +, 2688, 54205; cds-WP_004142122.1, NZ_KK737547.1, 446254, 447537, -, 1284, 1546721; cds-WP_004182478.1, NZ_KK737547.1, 447692, 449011, +, 1320, 213615; cds-WP_021441452.1, NZ_KK737547.1, 449008, 449964, +, 957, 34390; cds-WP_002895358.1, NZ_KK737547.1, 450026, 450751, +, 726, 210801; cds-AF52_RS27965, NZ_KK737547.1, 450894, 451647, +, 754, 560431; cds-WP_160333877.1, NZ_KK737547.1, 451644, 452582, +, 939, 1510645; cds-WP_032428723.1, NZ_KK737547.1, 452579, 454105, +, 1527, 889768; cds-WP_004147672.1, NZ_KK737547.1, 454204, 455586, +, 1383, 206692; cds-WP_002895575.1, NZ_KK737547.1, 456305, 456781, -, 477, 17200; cds-WP_004223758.1, NZ_KK737547.1, 456852, 458141, -, 1290, 94796; cds-WP_032429877.1, NZ_KK737547.1, 458355, 459395, +, 1041, 61569; cds-WP_004183475.1, NZ_KK737547.1, 459392, 460549, +, 1158, 48014; cds-WP_032429878.1, NZ_KK737547.1, 460533, 461288, +, 756, 10440; cds-WP_002895648.1, NZ_KK737547.1, 461281, 462003, +, 723, 39078; cds-WP_004152853.1, NZ_KK737547.1, 461945, 462667, -, 723, 8646; cds-WP_002895652.1, NZ_KK737547.1, 462754, 462966, -, 213, 42201; cds-WP_004147678.1, NZ_KK737547.1, 463378, 465399, +, 2022, 1099513; cds-WP_032429879.1, NZ_KK737547.1, 465648, 466745, -, 1098, 68781; cds-WP_002895659.1, NZ_KK737547.1, 466749, 467420, -, 672, 103988; cds-WP_021441454.1, NZ_KK737547.1, 467420, 469060, -, 1641, 27740; cds-WP_004142092.1, NZ_KK737547.1, 469165, 470070, -, 906, 4685; cds-WP_002895665.1, NZ_KK737547.1, 470405, 471394, +, 990, 198020; cds-WP_021441455.1, NZ_KK737547.1, 471418, 471930, +, 513, 62327; cds-WP_002895670.1, NZ_KK737547.1, 471934, 472422, +, 489, 35748; cds-WP_002895672.1, NZ_KK737547.1, 472415, 472663, +, 249, 1552; cds-WP_002895674.1, NZ_KK737547.1, 472665, 473117, +, 453, 11003; cds-WP_004142079.1, NZ_KK737547.1, 473163, 473867, +, 705, 105515; cds-WP_032429880.1, NZ_KK737547.1, 474048, 475013, -, 966, 56829; cds-WP_032429882.1, NZ_KK737547.1, 475010, 476254, -, 1245, 51518; cds-WP_004179098.1, NZ_KK737547.1, 476251, 477012, -, 762, 8279; cds-WP_004176793.1, NZ_KK737547.1, 477144, 477581, +, 438, 111954; cds-WP_004142068.1, NZ_KK737547.1, 477642, 478748, -, 1107, 131060; cds-WP_004212202.1, NZ_KK737547.1, 478878, 480011, -, 1134, 40528; cds-WP_032429883.1, NZ_KK737547.1, 480001, 481740, -, 1740, 193594; cds-WP_002895745.1, NZ_KK737547.1, 481733, 482728, -, 996, 138867; cds-WP_002895746.1, NZ_KK737547.1, 482728, 483387, -, 660, 42359; cds-WP_004223785.1, NZ_KK737547.1, 483461, 484384, -, 924, 8974; cds-WP_004142057.1, NZ_KK737547.1, 484376, 484513, +, 138, 0; cds-WP_019705008.1, NZ_KK737547.1, 484801, 486132, +, 1332, 328; cds-AF52_RS27125, NZ_KK737547.1, 486247, 486348, -, 102, 3043; cds-WP_032429885.1, NZ_KK737547.1, 488364, 488701, -, 338, 32043; cds-WP 002895750.1, NZ_KK737547.1, 488735, 489484, +, 750, 459; cds-WP_002895753.1, NZ_KK737547.1, 489498, 490343, +, 846, 2929; cds-WP_002895757.1, NZ_KK737547.1, 490343, 491335, +, 993, 0; cds-WP_032429886.1, NZ_KK737547.1, 491550, 492905, +, 1356, 284327; cds-WP_004147693.1, NZ_KK737547.1, 493093, 495249, +, 2157, 226505; cds-WP_004179105.1, NZ_KK737547.1, 495279, 496247, +, 969, 35195; cds-WP_004176772.1, NZ_KK737547.1, 496357, 496617, -, 261, 217080; cds-WP_032428726.1, NZ_KK737547.1, 496903, 497169, -, 267, 3502; cds-WP_004183485.1, NZ_KK737547.1, 497190, 497450, -, 261, 651; cds-WP_016529094.1, NZ_KK737547.1, 497590, 498204, -, 615, 99399; cds-WP_021441457.1, NZ_KK737547.1, 498301, 499215, +, 915, 61245; cds-WP_021441458.1, NZ_KK737547.1, 499218, 501413, -, 2196, 57906; cds-WP_004142040.1, NZ_KK737547.1, 501525, 502247, -, 723, 627524; cds-WP_002895837.1, NZ_KK737547.1, 502244, 502903, -, 660, 843647; cds-WP_002895839.1, NZ_KK737547.1, 503029, 503775, -, 747, 3283577; cds-WP_002895841.1, NZ_KK737547.1, 504184, 504687, -, 504, 247067; cds-WP_002895842.1, NZ_KK737547.1, 504925, 505812, -, 888, 25043; cds-WP_002895845.1, NZ_KK737547.1, 506164, 506676, +, 513, 1191159; cds-WP_021441460.1, NZ_KK737547.1, 506767, 508347, -, 1581, 605016; cds-WP_002895851.1, NZ_KK737547.1, 508616, 508741, -, 126, 141450; cds-WP_004176765.1, NZ_KK737547.1, 508927, 509400, +, 474, 69952; cds-WP_021441462.1, NZ_KK737547.1, 509397, 510509, +, 1113, 241712; cds-WP_004176762.1, NZ_KK737547.1, 510679, 512055, +, 1377, 379138; cds-WP_004151705.1, NZ_KK737547.1, 512124, 512840, +, 717, 62810; cds-WP_002895871.1, NZ_KK737547.1, 512986, 513900, -, 915, 516696; cds-WP_002895874.1, NZ_KK737547.1, 514101, 514886, -, 786, 60490; cds-WP_002895876.1, NZ_KK737547.1, 515065, 516657, +, 1593, 379598; cds-WP_021441465.1, NZ_KK737547.1, 517192, 519555, -, 2364, 39; cds-WP_004183491.1, NZ_KK737547.1, 519571, 520872, -, 1302, 2087; cds-WP_004219745.1, NZ_KK737547.1, 521115, 522245, +, 1131, 50133; cds-WP_004176744.1, NZ_KK737547.1, 522242, 523507, -, 1266, 95866; cds-WP_004212174.1, NZ_KK737547.1, 523647, 524462, -, 816, 290108; cds-WP_032429888.1, NZ_KK737547.1, 524639, 527071, -, 2433, 0; cds-WP_004183492.1, NZ_KK737547.1, 527076, 527975, -, 900, 2767; cds-WP_004141998.1, NZ_KK737547.1, 528130, 528885, -, 756, 46701; cds-WP_032429890.1, NZ_KK737547.1, 528885, 530120, -, 1236, 33666; cds-WP_004147725.1, NZ_KK737547.1, 530356, 531297, +, 942, 19509; cds-WP_004151710.1, NZ_KK737547.1, 531315, 533174, +, 1860, 280532; cds-WP_012068490.1, NZ_KK737547.1, 533207, 534748, +, 1542, 426411; cds-WP_002892997.1, NZ_KK737547.1, 534795, 535715, +, 921, 92962; cds-WP_004147729.1, NZ_KK737547.1, 535717, 536628, +, 912, 25599; cds-WP_032429891.1, NZ_KK737547.1, 537149, 538474, -, 1326, 528772; cds-WP_004176729.1, NZ_KK737547.1, 538707, 539948, -, 1242, 1387271; cds-WP_004151712.1, NZ_KK737547.1, 540327, 540710, +, 384, 72067; cds-WP_029884283.1, NZ_KK737547.1, 540826, 541935, +, 1110, 76782; cds-WP_004176727.1, NZ_KK737547.1, 541938, 542564, -, 627, 31119; cds-AF52_RS27135, NZ_KK737547.1, 542764, 542887, -, 124, 11223; cds-WP_171290763.1, NZ_KK737547.1, 551069, 551268, -, 200, 8854; cds-WP_179139609.1, NZ_KK737547.1, 552619, 552832, +, 214, 13777; cds-AF52_RS11970, NZ_KK737547.1, 559347, 559565, +, 219, 13068; cds-WP_032428727.1, NZ_KK737547.1, 559740, 559868, +, 129, 0; cds-WP_032429893.1, NZ_KK737547.1, 560520, 561722, +, 1203, 590429; cds-WP_004151717.1, NZ_KK737547.1, 561761, 562519, -, 759, 209211; cds-WP_004141974.1, NZ_KK737547.1, 562621, 563214, -, 594, 189315; cds-WP_004141972.1, NZ_KK737547.1, 563542, 564774, +, 1233, 64037; cds-WP_004141971.1, NZ_KK737547.1, 564812, 565624, -, 813, 275497; cds-WP_004151719.1, NZ_KK737547.1, 565624, 566826, -, 1203, 58742; cds-WP_032429895.1, NZ_KK737547.1, 566909, 567472, +, 564, 8191; cds-WP_032429979.1, NZ_KK737547.1, 567650, 569335, -, 1686, 93104; cds-WP_002896351.1, NZ_KK737547.1, 569602, 569985, +, 384, 54043; cds-WP_002896352.1, NZ_KK737547.1, 569992, 570255, -, 264, 56774; cds-WP_004179132.1, NZ_KK737547.1, 570458, 570745, +, 288, 69385; cds-WP_004179133.1, NZ_KK737547.1, 570729, 571451, +, 723, 16551; cds-WP_002896363.1, NZ_KK737547.1, 571566, 572468, +, 903, 33061; cds-WP_002896365.1, NZ_KK737547.1, 572557, 573036, +, 480, 179856; cds-WP_002896368.1, NZ_KK737547.1, 573385, 574497, +, 1113, 62099; cds-WP_002896370.1, NZ_KK737547.1, 574661, 575794, +, 1134, 114210; cds-WP_002896371.1, NZ_KK737547.1, 575805, 576758, +, 954, 2384; cds-WP_002896372.1, NZ_KK737547.1, 576755, 577600, +, 846, 31102; cds-WP_002896376.1, NZ_KK737547.1, 577658, 578146, +, 489, 106634; cds-WP_004147758.1, NZ_KK737547.1, 578188, 579315, +, 1128, 130123; cds-WP_002896380.1, NZ_KK737547.1, 579394, 580110, +, 717, 21539; cds-WP_002896382.1, NZ_KK737547.1, 580107, 581579, +, 1473, 12453; cds-WP_002896384.1, NZ_KK737547.1, 581666, 582397, -, 732, 93184; cds-WP_004141917.1, NZ_KK737547.1, 582581, 583249, -, 669, 271337; cds-WP_002896386.1, NZ_KK737547.1, 583249, 583965, -, 717, 96170; cds-WP_002896390.1, NZ_KK737547.1, 583972, 584703, -, 732, 244487; cds-WP_002896392.1, NZ_KK737547.1, 584724, 585452, -, 729, 159177; cds-WP_002896394.1, NZ_KK737547.1, 585679, 586194, -, 516, 90003; cds-AF52_RS11825, NZ_KK737547.1, 587073, 587345, +, 273, 388; cds-WP_013815099.1-37, NZ_KK737547.1, 587378, 588346, +, 969, 95238; cds-AF52_RS11815, NZ_KK737547.1, 588400, 589278, +, 879, 31580; cds-WP_032429899.1, NZ_KK737547.1, 589310, 590140, +, 831, 143774; cds-WP_002896399.1, NZ_KK737547.1, 590137, 591150, -, 1014, 311737; cds-WP_004209682.1, NZ_KK737547.1, 591238,
592680, -, 1443, 61543; cds-WP _032429901.1, NZ_KK737547.1, 592691, 593692, -, 1002, 57611; cds-WP _004176700.1, NZ_KK737547.1, 593731, 595449, -, 1719, 184566; cds-WP_002896408.1, NZ_KK737547.1, 595601, 596035, +, 435, 2159; cds-WP _004147771.1, NZ_KK737547.1, 596247, 597215, -, 969, 0; cds-WP _004176696.1, NZ_KK737547.1, 597226, 598878, -, 1653, 226; cds-AF52_RS11770, NZ_KK737547.1, 599022, 599681, -, 660, 5904; cds-WP _013815099.1-38, NZ_KK737547.1, 599738, 600706, -, 969, 92460; cds-AF52_RS11760, NZ_KK737547.1, 600739, 600987, -, 249, 13780; cds-WP _032429902.1, NZ_KK737547.1, 601103, 601798, -, 696, 2582; cds-WP _013815099.1-39, NZ_KK737547.1, 602061, 603029, -, 969, 83115; cds-WP _032429903.1, NZ_KK737547.1, 603283, 604941, +, 1659, 154950; cds-WP_002896440.1, NZ_KK737547.1, 605088, 606203, +, 1116, 510930; cds-WP_032429904.1, NZ_KK737547.1, 606200, 608140, +, 1941, 227455; cds-WP _002896516.1, NZ_KK737547.1, 608217, 608438, -, 222, 179488; cds-WP _002896520.1, NZ_KK737547.1, 608764, 609081, +, 318, 738868; cds-WP_002896522.1, NZ_KK737547.1, 609112, 611391, +, 2280, 2753192; cds-WP _001040187.1, NZ_KK737547.1, 611511, 611729, -, 219, 393746; cds-WP _002898014.1, NZ_KK737547.1, 612083, 612784, -, 702, 135965; cds-WP _004224003.1, NZ_KK737547.1, 612829, 614550, -, 1722, 116655; cds-WP_032429906.1, NZ_KK737547.1, 614551, 616317, -, 1767, 305259; cds-WP _002898019.1, NZ_KK737547.1, 616432, 617400, -, 969, 650347; cds-WP _000228469.1, NZ_KK737547.1, 617933, 618427, +, 495, 345735; cds-AF52_RS0123915, NZ_KK737547.1, 618563, 622808, +, 4246, 1364564; cds-WP_002898132.1, NZ_KK737547.1, 622931, 623542, +, 612, 356677; cds-WP _004179165.1, NZ_KK737547.1, 623551, 624894, +, 1344, 837337; cds-WP _002898139.1, NZ_KK737547.1, 624985, 626277, +, 1293, 3143502; cds-WP_004147794.1, NZ_KK737547.1, 626478, 628916, +, 2439, 5353; cds-WP_004150845.1, NZ_KK737547.1, 628927, 629544, +, 618, 13769; cds-WP _004150844.1, NZ_KK737547.1, 629546, 630409, +, 864, 0; cds-WP _032429909.1, NZ_KK737547.1, 630684, 631832, +, 1149, 192456; cds-WP_002898145.1, NZ_KK737547.1, 631995, 632735, -, 741, 45432; cds-WP _002898148.1, NZ_KK737547.1, 632927, 635209, -, 2283, 2677902; cds-WP _032429911.1, NZ_KK737547.1, 635261, 636118, -, 858, 159177; cds-WP_004150842.1, NZ_KK737547.1, 636504, 638264, -, 1761, 793281; cds-WP _002898152.1, NZ_KK737547.1, 638380, 639072, +, 693, 2304; cds-WP _004176687.1, NZ_KK737547.1, 639261, 640349, +, 1089, 245942; cds-WP_002898157.1, NZ_KK737547.1, 640423, 641706, +, 1284, 297945; cds-WP _002898160.1, NZ_KK737547.1, 641881, 642564, +, 684, 373879; cds-WP _002898162.1, NZ_KK737547.1, 642698, 644371, +, 1674, 41143787; cds-WP_002898165.1, NZ_KK737547.1, 644523, 644810, +, 288, 867236; cds-WP_032411196.1, NZ_KK737547.1, 645016, 647280, +, 2265, 1354; cds-WP_004201387.1, NZ_KK737547.1, 647317, 649065, +, 1749, 416393; cds-WP _016946992.1, NZ_KK737547.1, 649062, 650042, +, 981, 33582; cds-WP _032429912.1, NZ_KK737547.1, 650097, 651326, +, 1230, 100009; cds-WP_002898177.1, NZ_KK737547.1, 651390, 651572, +, 183, 79898; cds-WP _032429914.1, NZ_KK737547.1, 651569, 652315, +, 747, 215041; cds-WP _002898182.1, NZ_KK737547.1, 652453, 653346, +, 894, 1211; cds-WP_004141786.1, NZ_KK737547.1, 653323, 654102, -, 780, 38118; cds-WP_002898187.1, NZ_KK737547.1, 654224, 655024, +, 801, 191885; cds-WP _032429915.1, NZ_KK737547.1, 655021, 656343, +, 1323, 388587; cds-WP _004150840.1, NZ_KK737547.1, 656324, 657028, +, 705, 167868; cds-WP _004176682.1, NZ_KK737547.1, 657028, 661476, +, 4449, 1250961; cds-WP_002898195.1, NZ_KK737547.1, 661677, 663467, +, 1791, 269014; cds-WP_002898198.1, NZ_KK737547.1, 663770, 664321, +, 552, 1123889; cds-WP_002898200.1, NZ_KK737547.1, 664335, 664982, +, 648, 739570; cds-WP_002898202.1, NZ_KK737547.1, 665051, 666241, -, 1191, 1081062; cds-WP _004141771.1, NZ_KK737547.1, 666429, 667508, -, 1080, 1093646; cds-WP _032429917.1, NZ_KK737547.1, 668100, 669500, -, 1401, 936523; cds-AF52_RS11510, NZ_KK737547.1, 669581, 670140, -, 560, 53773; cds-WP _013815099.1-40, NZ_KK737547.1, 670203, 671171, -, 969, 83748; cds-WP_004179185.1, NZ_KK737547.1, 671515, 672726, +, 1212, 12093; cds-WP_023286202.1, NZ_KK737547.1, 672849, 674138, +, 1290, 1846; cds-WP_032429918.1, NZ_KK737547.1, 674163, 675401, +, 1239, 29855; cds-WP _004179190.1, NZ_KK737547.1, 675420, 676586, +, 1167, 6640; cds-WP _002898217.1, NZ_KK737547.1, 676629, 677831, -, 1203, 273890; cds-WP_002898220.1, NZ_KK737547.1, 678158, 680773, +, 2616, 2074497; cds-WP_004150838.1, NZ_KK737547.1, 680978, 681751, -, 774, 39185; cds-WP _004141755.1, NZ_KK737547.1, 681748, 682539, -, 792, 2; cds-WP_002898293.1, NZ_KK737547.1, 682550, 683695, -, 1146, 0; cds-WP _023284918.1, NZ_KK737547.1, 683692, 684654, -, 963, 103; cds-WP _032429920.1, NZ_KK737547.1, 684647, 685222, -, 576, 5723; cds-WP _002898303.1, NZ_KK737547.1, 685472, 686482, +, 1011, 270949; cds-WP _002898308.1, NZ_KK737547.1, 686652, 687194, +, 543, 173724; cds-WP_004179196.1, NZ_KK737547.1, 687191, 688300, -, 1110, 147560; cds-WP _004147847.1, NZ_KK737547.1, 688400, 690505, +, 2106, 407687; cds-WP _004147848.1, NZ_KK737547.1, 690518, 692425, +, 1908, 775594; cds-WP_004147849.1, NZ_KK737547.1, 692440, 693708, +, 1269, 312050; cds-WP _002898390.1, NZ_KK737547.1, 693698, 695335, +, 1638, 266739; cds-WP_002898392.1, NZ_KK737547.1, 695335, 695898, +, 564, 55751; cds-WP _002898396.1, NZ_KK737547.1, 696152, 696319, +, 168, 47894; cds-WP_002898398.1, NZ_KK737547.1, 696388, 696906, -, 519, 425771; cds-WP_032429922.1, NZ_KK737547.1, 696976, 698733, -, 1758, 859103; cds-WP _002898404.1, NZ_KK737547.1, 698919, 699371, +, 453, 10147; cds-WP _002898408.1, NZ_KK737547.1, 699503, 700573, -, 1071, 25234677; cds-WP _002898418.1, NZ_KK737547.1, 700926, 701435, -, 510, 581309; cds-WP _032429924.1, NZ_KK737547.1, 701784, 702380, +, 597, 5441; cds-WP_002898422.1, NZ_KK737547.1, 702394, 704529, -, 2136, 457626; cds-WP_002898425.1, NZ_KK737547.1, 704547, 704993, -, 447, 97253; cds-WP _004176653.1, NZ_KK737547.1, 705118, 707172, +, 2055, 166336; cds-WP _002898432.1, NZ_KK737547.1, 707184, 707642, -, 459, 143320; cds-WP_004144085.1, NZ_KK737547.1, 707797, 708210, +, 414, 87062; cds-WP _013815099.1-41, NZ_KK737547.1, 708377, 709345, -, 969, 90258; cds-AF52_RS0123935, NZ_KK737547.1, 709895, 712733, +, 2839, 98135; cds-WP_072001581.1, NZ_KK737547.1, 724264, 728283, +, 4020, 126506; cds-AF52_RS11335, NZ_KK737547.1, 728452, 729213, +, 762, 3722; cds-AF52_RS27170, NZ_KK737547.1, 729248, 729418, +, 171, 5587; cds-AF52_RS11330, NZ_KK737547.1, 730319, 730471, +, 153, 5558; cds-AF52_RS11325, NZ_KK737547.1, 730530, 733256, +, 2727, 1036; cds-WP _032429986.1, NZ_KK737547.1, 733403, 734278, +, 876, 24424; cds-WP_002898441.1, NZ_KK737547.1, 734321, 734638, -, 318, 79501; cds-WP_032429928.1, NZ_KK737547.1, 734698, 735900, -, 1203, 45747; cds-WP _004210995.1, NZ_KK737547.1, 736091, 736645, +, 555, 146; cds-WP _004183586.1, NZ_KK737547.1, 736663, 737451, +, 789, 37090; cds-WP_032429987.1, NZ_KK737547.1, 737500, 737781, +, 282, 85267; cds-WP_002898457.1, NZ_KK737547.1, 737778, 738107, -, 330, 19018; cds-WP _002898458.1, NZ_KK737547.1, 738196, 738855, -, 660, 1330575; cds-WP_032429929.1, NZ_KK737547.1, 739125, 740777, +, 1653, 21092; cds-AF52_RS26415, NZ_KK737547.1, 741134, 741886, -, 753, 164273; cds-WP_032429931.1, NZ_KK737547.1, 741867, 742055, +, 189, 23256; cds-WP_032429933.1, NZ_KK737547.1, 742225, 743190, -, 966, 78286; cds-WP_004211006.1, NZ_KK737547.1, 743248, 744696, -, 1449, 314711; cds-WP_002898571.1, NZ_KK737547.1, 744715, 745839, -, 1125, 170899; cds-WP_009308376.1, NZ_KK737547.1, 745876, 747483, -, 1608, 296541; cds-WP _032429935.1, NZ_KK737547.1, 747988, 749073, -, 1086, 1500; cds-WP_004221441.1, NZ_KK737547.1, 749156, 750118, +, 963, 2502; cds-WP_002898585.1, NZ_KK737547.1, 750115, 751395, -, 1281, 69322; cds-WP _032429937.1, NZ_KK737547.1, 751398, 752885, -, 1488, 161500; cds-WP _002898590.1, NZ_KK737547.1, 753206, 753763, -, 558, 1114762; cds-WP_020802183.1, NZ_KK737547.1, 753789, 754541, -, 753, 635242; cds-WP_002898595.1, NZ_KK737547.1, 754767, 756188, +, 1422, 611289; cds-WP _004141463.1, NZ_KK737547.1, 756542, 757933, +, 1392, 417647; cds-WP_002898600.1, NZ_KK737547.1, 758052, 758174, -, 123, 42488; cds-WP_021441517.1, NZ_KK737547.1, 758431, 758652, +, 222, 36440; cds-WP_004150831.1, NZ_KK737547.1, 758673, 759461, -, 789, 13776; cds-WP_004141456.1, NZ_KK737547.1, 759577, 760026, -, 450, 4346; cds-WP_004183618.1, NZ_KK737547.1, 760179, 761426, +, 1248, 259151; cds-WP_002898701.1, NZ_KK737547.1, 761495, 761722, -, 228, 47815; cds-WP _002898704.1, NZ_KK737547.1, 761743, 762339, -, 597, 165165; cds-WP_004224058.1, NZ_KK737547.1, 762556, 762687, -, 132, 218; cds-WP _002898708.1, NZ_KK737547.1, 762775, 762948, +, 174, 1458; cds-WP_004199515.1, NZ_KK737547.1, 763111, 764049, +, 939, 30679; cds-WP _004191019.1, NZ_KK737547.1, 764455, 765777, -, 1323, 16; cds-WP _004176611.1, NZ_KK737547.1, 765799, 766293, -, 495, 0; cds-WP _004141442.1, NZ_KK737547.1, 766304, 766894, -, 591, 341; cds-WP_004179289.1, NZ_KK737547.1, 766891, 767694, -, 804, 287; cds-WP_002898877.1, NZ_KK737547.1, 767691, 768083, -, 393, 521; cds-WP_004224061.1, NZ_KK737547.1, 768076, 768786, -, 711, 0; cds-WP _004179293.1, NZ_KK737547.1, 768786, 769877, -, 1092, 30553; cds-WP_002898883.1, NZ_KK737547.1, 770162, 770800, +, 639, 101810; cds-WP_002898886.1, NZ_KK737547.1, 770797, 771192, -, 396, 77265; cds-WP _032429939.1, NZ_KK737547.1, 771378, 775340, -, 3963, 3104138; cds-WP_013815099.1-42, NZ_KK737547.1, 775708, 776676, +, 969, 81545; cds-WP_032429941.1, NZ_KK737547.1, 776720, 777919, +, 1200, 132163; cds-AF52_RS0123960, NZ_KK737547.1, 777953, 778729, +, 777, 16000; cds-WP _020806188.1-3, NZ_KK737547.1, 778830, 779810, -, 981, 236518; cds-WP_032429943.1, NZ_KK737547.1, 779855, 781216, +, 1362, 601; cds-WP_021440205.1, NZ_KK737547.1, 781216, 781701, +, 486, 0; cds-WP _021440204.1, NZ_KK737547.1, 781711, 782727, +, 1017, 0; cds-WP _004143751.1, NZ_KK737547.1, 782737, 783510, +, 774, 600; cds-WP _002906464.1, NZ_KK737547.1, 783507, 784385, -, 879, 1299; cds-WP_148673683.1, NZ_KK737547.1, 784611, 787658, +, 3048, 10212; cds-WP_004143747.1, NZ_KK737547.1, 787670, 788554, +, 885, 70; cds-WP _002906457.1, NZ_KK737547.1, 788547, 789203, +, 657, 26; cds-AF52_RS25665, NZ_KK737547.1, 789256, 789581, -, 326, 38227; cds-WP _004175994.1, NZ_KK737547.1, 789578, 789763, -, 186, 63; cds-WP_004180084.1, NZ_KK737547.1, 789942, 790760, -, 819, 6021; cds-WP_021440202.1, NZ_KK737547.1, 790828, 791268, -, 441,11478; cds-WP_004180082.1, NZ_KK737547.1, 791533, 792687, +, 1155, 6624; cds-WP_020802157.1, NZ_KK737547.1, 792694, 793785, -, 1092, 2676; cds-WP _004184178.1, NZ_KK737547.1, 793795, 794781, -, 987, 72; cds-WP_021440201.1, NZ_KK737547.1, 794781, 796397, -, 1617, 1165; cds-WP_020324770.1, NZ_KK737547.1, 796394, 797242, -, 849, 116; cds-WP_023280140.1, NZ_KK737547.1, 797239, 798183, -, 945, 0; cds-WP _002906226.1, NZ_KK737547.1, 798194, 799816, -, 1623, 1452; cds-WP _002906221.1, NZ_KK737547.1, 799986, 800996, -, 1011, 317694; cds-WP_002906218.1, NZ_KK737547.1, 801249, 801848, +, 600, 614; cds-WP_004176019.1, NZ_KK737547.1, 802022, 802975, +, 954, 1688; cds-WP _032428292.1, NZ_KK737547.1, 803012, 804727, -, 1716, 22446; cds-WP_004200032.1, NZ_KK737547.1, 805090, 805776, -, 687, 30823; cds-WP_021440197.1, NZ_KK737547.1, 805773, 806468, -, 696, 81894; cds-WP_002906125.1, NZ_KK737547.1, 806652, 808349, -, 1698, 625582; cds-WP _002906122.1, NZ_KK737547.1, 808406, 808534, +, 129, 4931; cds-WP_004143727.1, NZ_KK737547.1, 808531, 808671, -, 141, 7305; cds-WP_032429948.1, NZ_KK737547.1, 808849, 809700, -, 852, 163; cds-WP_021440195.1, NZ_KK737547.1, 809814, 810707, +, 894, 50587; cds-WP_004180069.1, NZ_KK737547.1, 810775, 811020, -, 246, 10989; cds-WP _004151242.1, NZ_KK737547.1, 811217, 812131, -, 915, 444; cds-WP_004143718.1, NZ_KK737547.1, 812787, 812972, +, 186, 473; cds-WP_013815099.1-43, NZ_KK737547.1, 813024, 813992, +, 969, 89267; cds-WP_004143717.1, NZ_KK737547.1, 814404, 814619, +, 216, 0; cds-WP _023301896.1, NZ_KK737547.1, 814643, 815149, +, 507, 0; cds-WP _004143715.1, NZ_KK737547.1, 815166, 815348, +, 183, 0; cds-WP_021440193.1, NZ_KK737547.1, 815388, 817013, +, 1626, 232357; cds-WP_032429949.1, NZ_KK737547.1, 817010, 818005, -, 996, 1; cds-WP _004148482.1, NZ_KK737547.1, 818324, 818770, -, 447, 43; cds-WP_021440191.1, NZ_KK737547.1, 818779, 819282, -, 504, 10376; cds-WP _002906035.1,
NZ_KK737547.1, 819427, 819831, +, 405, 35; cds-WP _002906033.1, NZ_KK737547.1, 819847, 819993, +, 147, 357; cds-WP_002906030.1, NZ_KK737547.1, 820107, 820538, +, 432, 2763; cds-WP_021440189.1, NZ_KK737547.1, 820643, 821512, +, 870, 3678; cds-WP _004184165.1, NZ_KK737547.1, 821514, 822200, -, 687, 2391; cds-WP _004143707.1, NZ_KK737547.1, 822190, 822372, -, 183, 53; cds-WP _004151892.1, NZ_KK737547.1, 822422, 823345, +, 924, 0; cds-WP _023282626.1, NZ_KK737547.1, 823595, 824512, +, 918, 29284; cds-WP_023280134.1, NZ_KK737547.1, 824828, 827128, +, 2301, 136507; cds-WP_002906016.1, NZ_KK737547.1, 827278, 828159, +, 882, 78850; cds-WP _004180040.1, NZ_KK737547.1, 828422, 829828, -, 1407, 17110; cds-WP_002906011.1, NZ_KK737547.1, 830416, 831096, +, 681, 27589; cds-AF52_RS0123990, NZ_KK737547.1, 831194, 832255, +, 1062, 256954; cds-WP_004180028.1, NZ_KK737547.1, 832290, 834467, -, 2178, 110646; cds-WP _004184162.1, NZ_KK737547.1, 834736, 835668, -, 933, 14627; cds-WP_002905764.1, NZ_KK737547.1, 835768, 836202, +, 435, 0; cds-WP_004143692.1, NZ_KK737547.1, 836212, 836688, +, 477, 0; cds-WP_002905759.1, NZ_KK737547.1, 836887, 838797, +, 1911, 4406; cds-WP _004189919.1, NZ_KK737547.1, 838910, 839647, +, 738, 9875; cds-WP_004189922.1, NZ_KK737547.1, 839644, 841929, -, 2286, 18511; cds-WP_004184158.1, NZ_KK737547.1, 841929, 843071, -, 1143, 168494; cds-WP _004143686.1, NZ_KK737547.1, 843058, 843336, -, 279, 35446; cds-WP _004143685.1, NZ_KK737547.1, 843339, 844094, -, 756, 81893; cds-WP_004148467.1, NZ_KK737547.1, 844088, 845014, -, 927, 101614; cds-WP _002905689.1, NZ_KK737547.1, 845069, 845140, -, 72, 25427; cds-WP _021440182.1, NZ_KK737547.1, 845277, 846296, -, 1020, 61209; cds-WP_032429953.1, NZ_KK737547.1, 846539, 847531, +, 993, 542; cds-WP _004176065.1, NZ_KK737547.1, 847618, 848358, +, 741, 4849; cds-WP_032428291.1, NZ_KK737547.1, 848355, 849698, +, 1344, 1561; cds-WP _004184155.1, NZ_KK737547.1, 849801, 850991, +, 1191, 9; cds-WP_032429955.1, NZ_KK737547.1, 851905, 852798, -, 894, 3604; cds-WP_187079193.1, NZ_KK737547.1, 852992, 854473, -, 1482, 299036; cds-WP _002905540.1, NZ_KK737547.1, 854483, 855868, -, 1386, 462698; cds-WP _013815099.1-44, NZ_KK737547.1, 856165, 857133, +, 969, 77334; cds-WP_021440179.1, NZ_KK737547.1, 857241, 858176, -, 936, 121117; cds-WP _032429957.1, NZ_KK737547.1, 858201, 858941, -, 741, 44000; cds-WP_004189949.1, NZ_KK737547.1, 858938, 859666, -, 729, 95471; cds-WP_004189951.1, NZ_KK737547.1, 859663, 860358, -, 696, 313058; cds-AF52_RS28000, NZ_KK737547.1, 860529, 860615, +, 87, 14657; cds-WP_002905531.1, NZ_KK737547.1, 860775, 861881, +, 1107, 21844; cds-WP _021440176.1, NZ_KK737547.1, 861919, 863091, -, 1173, 57973; cds-WP _032411533.1, NZ_KK737547.1, 863105, 864028, -, 924, 45503; cds-WP_004148452.1, NZ_KK737547.1, 864221, 865261, -, 1041, 5433; cds-WP _032429958.1, NZ_KK737547.1, 865660, 866916, +, 1257, 162978; cds-WP _021440173.1, NZ_KK737547.1, 867015, 868325, -, 1311, 3895; cds-WP_021440172.1, NZ_KK737547.1, 868427, 869923, -, 1497, 9155; cds-WP_004179987.1, NZ_KK737547.1, 870068, 870784, +, 717, 3797; cds-AF52_RS10695, NZ_KK737547.1, 870784, 871655, +, 872, 28316; cds-WP _004179983.1, NZ_KK737547.1, 871540, 872472, +, 933, 165382; cds-WP _004184144.1, NZ_KK737547.1, 872561, 873352, -, 792, 292; cds-WP_004189983.1, NZ_KK737547.1, 873462, 874229, +, 768, 3849; cds-WP _004176101.1, NZ_KK737547.1, 874432, 874656, +, 225, 34680; cds-AF52_RS25690, NZ_KK737547.1, 874992, 875342, +, 351, 25784; cds-WP_002882540.1, NZ_KK737548.1, 3401, 4102, +, 702, 78994; cds-WP _002882539.1, NZ KK737548.1, 4115, 5512, +, 1398, 229461; cds-WP_004187944.1, NZ_KK737548.1, 5509, 6501, -, 993, 446202; cds-WP_187079194.1, NZ_KK737548.1, 6505, 7437, -, 933, 349287; cds-WP _002882536.1, NZ_KK737548.1, 7538, 8428, -, 891, 298510; cds-WP _002882531.1, NZ_KK737548.1, 8456, 9421, -, 966, 124181; cds-WP _002882529.1, NZ KK737548.1, 9427, 10932, -, 1506, 304766; cds-WP_002882527.1, NZ KK737548.1, 10943, 11362, -, 420, 27382; cds-WP_002882520.1, NZ_KK737548.1, 11548, 13416, -, 1869, 242639; cds-WP _004151555.1, NZ_KK737548.1, 13634, 15133, +, 1500, 201649; cds-WP _002882517.1, NZ_KK737548.1, 15130, 16578, +, 1449, 152389; cds-WP _002882514.1, NZ KK737548.1, 16582, 17574, -, 993, 3762; cds-WP_002882510.1, NZ_KK737548.1, 17726, 18184, +, 459, 113853; cds-WP_004144988.1, NZ_KK737548.1, 18283, 18723, +, 441, 332983; cds-WP _004144989.1, NZ KK737548.1, 19105, 20994, +, 1890, 2127687; cds-WP _004151554.1, NZ KK737548.1, 21110, 21733, +, 624, 258106; cds-WP_013815099.1-45, NZ_KK737548.1, 22264, 23232, +, 969, 117299; cds-WP _004144991.1, NZ_KK737548.1, 23416, 23796, +, 381, 199415; cds-WP_004151552.1, NZ_KK737548.1, 23804, 24619, +, 816, 4392704; cds-WP_000429386.1, NZ KK737548.1, 24669, 24908, +, 240, 2872007; cds-WP _004107293.1, NZ_KK737548.1, 24959, 25429, +, 471, 6642779; cds-WP_004144994.1, NZ KK737548.1, 25444, 25977, +, 534, 6104178; cds-WP_004144995.1, NZ KK737548.1, 25990, 27531, +, 1542, 18931573; cds-WP _004144996.1, NZ KK737548.1, 27582, 28445, +, 864,10231881; cds-WP_004144997.1, NZ KK737548.1, 28472, 29854, +, 1383, 16958859; cds-WP_001251971.1, NZ_KK737548.1, 29875, 30294, +, 420, 2306219; cds-WP _004151550.1, NZ_KK737548.1, 30991, 32361, +, 1371, 844003; cds-WP _004150309.1, NZ_KK737548.1, 32545, 34374, +, 1830, 2220161; cds-WP_004150308.1, NZ_KK737548.1, 34711, 35751, +, 1041, 737210; cds-WP_004145004.1, NZ_KK737548.1, 35880, 36839, +, 960, 178859; cds-WP _004151547.1, NZ_KK737548.1, 36839, 37729, +, 891, 332626; cds-WP _004145006.1, NZ_KK737548.1, 37777, 38550, +, 774, 576806; cds-WP _004145007.1, NZ_KK737548.1, 38601, 39326, +, 726, 156276; cds-AF52_RS10495, NZ KK737548.1, 39804, 40754, +, 951, 6868; cds-WP _013815099.1-46, NZ_KK737548.1, 40806, 41774, -, 969, 75849; cds-AF52_RS10485, NZ_KK737548.1, 41809, 42504, +, 696, 283; cds-WP _032430005.1, NZ KK737548.1, 42543, 43709, +, 1167, 27371; cds-WP_004895208.1, NZ_KK737548.1, 43759, 44478, +, 720, 10530; cds-WP_004145102.1, NZ_KK737548.1, 44558, 45223, -, 666, 42385; cds-WP _004145100.1, NZ_KK737548.1, 45393, 46730, +, 1338, 224594; cds-WP _004151543.1, NZ_KK737548.1, 46776, 47342, -, 567, 234698; cds-WP_004145098.1, NZ_KK737548.1, 47379, 47954, -, 576, 113809; cds-WP _004211197.1, NZ KK737548.1, 48264, 49223, -, 960, 17183; cds-WP_004181598.1, NZ KK737548.1, 49198, 50376, -, 1179, 56333; cds-WP _004151539.1, NZ_KK737548.1, 50476, 50895, -, 420, 847; cds-WP _004151538.1, NZ_KK737548.1, 51015, 52379, -, 1365, 242071; cds-WP_004150295.1, NZ_KK737548.1, 52574, 54220, -, 1647, 8657178; cds-WP _004151536.1, NZ_KK737548.1, 54223, 54480, -, 258, 980675; cds-WP_004151535.1, NZ KK737548.1, 54444, 54803, -, 360, 1854190; cds-WP_000831330.1, NZ_KK737548.1, 54819, 54959, -, 141, 705189; cds-WP _004151534.1, NZ KK737548.1, 55581, 56984, +, 1404, 1092569; cds-WP _004145090.1, NZ_KK737548.1, 56989, 58089, +, 1101, 598571; cds-WP_004150293.1, NZ_KK737548.1, 58297, 59370, +, 1074, 658733; cds-WP_004173845.1, NZ KK737548.1, 59399, 61813, +, 2415, 2357890; cds-WP_050484285.1, NZ_KK737548.1, 62015, 62869, +, 855, 137808; cds-WP_020806188.1-4, NZ_KK737548.1, 62970, 63950, -, 981, 237712; cds-AF52_RS25730, NZ KK737548.1, 64019, 64090, +, 72, 51; cds-WP_021440944.1, NZ_KK737548.1, 64268, 65605, +, 1338, 7543; cds-WP_004150284.1, NZ_KK737548.1, 65657, 66883, +, 1227, 28440; cds-WP_004151524.1, NZ_KK737548.1, 66885, 67217, -, 333, 13001; cds-WP_004151523.1, NZ KK737548.1, 67532, 67945, +, 414, 6433373; cds-WP_004145074.1, NZ_KK737548.1, 68062, 68490, +, 429, 7777986; cds-WP_009308837.1, NZ KK737548.1, 68627, 69451, +, 825, 172207; cds-WP_019705399.1, NZ_KK737548.1, 69553, 71214, +, 1662, 227816; cds-WP_004151520.1, NZ_KK737548.1, 71206, 71928, -, 723, 5456; cds-WP_002923307.1, NZ KK737548.1, 72227, 73849, +, 1623, 42435; cds-WP _002923306.1, NZ_KK737548.1, 73846, 75168, +, 1323, 48015; cds-AF52_RS24880, NZ KK737548.1, 75270, 75617, +, 348, 7700; cds-WP_002923304.1, NZ_KK737548.1, 75607, 75969, +, 363, 18926; cds-WP _002923302.1, NZ_KK737548.1, 76044, 76466, +, 423, 3345; cds-WP_020723563.1, NZ_KK737548.1, 76470, 77798, -, 1329, 7215; cds-WP_004173855.1, NZ KK737548.1, 77816, 79153, -, 1338, 7806; cds-WP _032410116.1, NZ KK737548.1, 79245, 79391, +, 147, 17; cds-WP_032430010.1, NZ_KK737548.1, 79379, 80302, +, 924, 311334; cds-WP _004181587.1, NZ_KK737548.1, 80462, 81646, -, 1185, 159442; cds-WP_032430012.1, NZ_KK737548.1, 81822, 82655, -, 834, 117320; cds-WP _002923294.1, NZ KK737548.1, 82724, 83170, -, 447, 204845; cds-WP _004145059.1, NZ KK737548.1, 83261, 83380, -, 120, 192891; cds-WP _004150272.1, NZ KK737548.1, 83833, 84000, -, 168, 1951; cds-WP _002923286.1, NZ KK737548.1, 84013, 84108, +, 96, 1209; cds-WP _004173858.1, NZ_KK737548.1, 84213, 85901, +, 1689, 227731; cds-WP _002923283.1, NZ_KK737548.1, 85905, 86192, +, 288, 73917; cds-WP _002923282.1, NZ KK737548.1, 86343, 86936, +, 594, 13516; cds-AF52_RS10285, NZ KK737548.1, 86954, 88434, +, 1481, 8631; cds-WP_004145056.1, NZ KK737548.1, 88446, 89774, +, 1329, 602; cds-WP _002923280.1, NZ_KK737548.1, 89920, 91311, +, 1392, 110; cds-WP _004901545.1, NZ KK737548.1, 91448, 91900, +, 453, 72093; cds-WP _004173862.1, NZ KK737548.1, 92029, 92352, -, 324, 20629; cds-AF52_RS10260, NZ KK737548.1, 92336, 92696, -, 361, 60335; cds-WP_002923205.1, NZ_KK737548.1, 92908, 94101, +, 1194, 25438; cds-WP_021440939.1, NZ_KK737548.1, 94098, 94907, -, 810, 3136; cds-WP _021440938.1, NZ_KK737548.1, 94917, 96020, -, 1104, 33158; cds-WP _004145049.1, NZ KK737548.1, 96162, 96881, +, 720, 72845; cds-WP _021440937.1, NZ KK737548.1, 97058, 98071, +, 1014, 9869; cds-WP _004150258.1, NZ KK737548.1, 98077, 99189, +, 1113, 2439; cds-WP _004150257.1, NZ_KK737548.1, 99189, 100049, +, 861, 9739; cds-WP_004145044.1, NZ_KK737548.1, 100052, 100849, +, 798, 12392; cds-WP _032422633.1, NZ KK737548.1, 101047, 102426, -, 1380, 0; cds-WP _002923158.1, NZ_KK737548.1, 102571, 102873, -, 303, 0; cds-WP_002923156.1, NZ KK737548.1, 102971, 104293, -, 1323, 8830; cds-WP_004145041.1, NZ_KK737548.1, 104305, 104619, -, 315, 18809; cds-WP_002923154.1, NZ_KK737548.1, 104913, 105854, +, 942, 30657; cds-WP _032426549.1, NZ KK737548.1, 106066, 107436, +, 1371, 258; cds-WP_002923152.1, NZ_KK737548.1, 107450, 108712, +, 1263, 1191; cds-WP _032430013.1, NZ_KK737548.1, 108709, 109935, -, 1227, 21143; cds-WP _002923146.1, NZ KK737548.1, 110063, 110803, +, 741, 3402; cds-WP_032430014.1, NZ_KK737548.1, 110800, 111513, +, 714, 0; cds-WP _032428449.1, NZ _KK737548.1, 111514, 112599, +, 1086, 8784; cds-WP _032428448.1, NZ_KK737548.1, 112602, 113465, +, 864, 16714; cds-WP_004145033.1, NZ _KK737548.1, 113469, 113744, -, 276, 12418; cds-WP _004234160.1, NZ_KK737548.1, 113866, 114804, -, 939, 134513; cds-WP _004150245.1, NZ_KK737548.1, 114920, 116353, -, 1434, 307706; cds-WP_021440928.1, NZ KK737548.1, 116378, 117280, -, 903, 326926; cds-WP_002923107.1, NZ_KK737548.1, 117443, 118606, -, 1164, 0; cds-WP_004889164.1, NZ_KK737548.1, 118652, 120016, -, 1365, 139; cds-WP _004151514.1, NZ_KK737548.1, 120020, 123127, -, 3108, 1365; cds-WP_016529678.1, NZ KK737548.1, 123127, 124251, -, 1125, 468; cds-WP_032412474.1, NZ_KK737548.1, 124458, 124580, +, 123, 122; cds-WP_032428455.1, NZ_KK737548.1, 124540, 124656, -, 117, 0; cds-WP_004173879.1, NZ_KK737548.1, 124631, 125047, +, 417, 1319; cds-WP _004181564.1, NZ_KK737548.1, 125551, 126198, -, 648, 68804; cds-WP_032430016.1, NZ KK737548.1, 126279, 127772, +, 1494, 10140; cds-WP_002923020.1, NZ_KK737548.1, 127769, 128683, -, 915, 8578; cds-WP _021440922.1, NZ_KK737548.1, 128779, 129405, +, 627, 2111; cds-WP _021440921.1, NZ _KK737548.1, 129408, 129878, +, 471, 15895; cds-WP _004145017.1, NZ_KK737548.1, 129899, 130435, -, 537, 32865; cds-WP_004150236.1, NZ_KK737548.1, 130552, 131457, +, 906, 48766; cds-WP _004173884.1, NZ KK737548.1, 131586, 132395, +, 810, 85509; cds-WP_032430018.1, NZ_KK737548.1, 132420, 133274, +, 855, 11939; cds-WP_021440918.1, NZ _KK737548.1, 133264, 133824, -, 561, 2869; cds-WP _004173886.1, NZ_KK737548.1, 133990, 134769, +, 780, 19165; cds-WP _004145010.1, NZ_KK737548.1, 134791,135744, +, 954, 8401; cds-WP _004901462.1, NZ _KK737548.1, 135741, 136790, -, 1050, 3168; cds-WP_004873113.1, NZ_KK737548.1, 137163, 137861, +, 699, 61071; cds-WP_021440916.1, NZ_KK737548.1, 137912, 138202, -, 291, 30534; cds-WP_002922964.1, NZ_KK737548.1, 138204, 139211, -, 1008, 13865; cds-WP _002922963.1, NZ KK737548.1, 139324, 139791, -, 468, 1864; cds-WP _088296409.1, NZ_KK737548.1;NZ_KK737548.1, 140181;140442, 140442;141343, +;+, 1163, 186054; cds-
WP_108443077.1, NZ_KK737548.1, 141410, 141619, +, 210, 32093; cds-AF52_RS0124095, NZ_KK737548.1, 141929, 142276, -, 348, 43549; cds-AF52_RS0124105, NZ_KK737548.1, 142332, 143029, +, 698, 367704; cds-WP _021440909.1, NZ _KK737548.1, 143089, 144468, +, 1380, 100124; cds-WP_004173889.1, NZ_KK737548.1, 144515, 145237, -, 723, 13044; cds-AF52_RS0124110, NZ_KK737548.1, 145393, 147711, -, 2319, 18830; cds-WP_002922962.1, NZ_KK737548.1, 147724, 149118, -, 1395, 3096; cds-WP _032428443.1, NZ_KK737548.1, 149805, 151016, +, 1212, 363760; cds-WP_013815099.1-47, NZ_KK737548.1, 151813, 152781, -, 969, 114996; cds-WP_148673684.1, NZ_KK737548.1, 153177, 153419, -, 243, 2961; cds-WP_013815099.1-48, NZ_KK737548.1, 153544, 154512, -, 969, 102286; cds-WP_050484287.1, NZ_KK737548.1, 154501, 155697, +, 1197, 36374; cds-WP_002922944.1, NZ_KK737548.1, 155812, 157323, -, 1512, 10905803; cds-WP _002922941.1, NZ_KK737548.1, 157345, 158196, -, 852, 2728325; cds-WP _004150222.1, NZ_KK737548.1, 158639, 158884, +, 246, 295574; cds-WP_002922936.1, NZ_KK737548.1, 159036, 160025, +, 990, 312131; cds-WP_020803798.1, NZ_KK737548.1, 160188, 160958, +, 771, 12515; cds-WP _019724988.1, NZ_KK737548.1, 161023, 162327, +, 1305, 14867; cds-WP _002922909.1, NZ_KK737548.1, 162420, 163187, -, 768, 1279287; cds-WP_002922908.1, NZ _KK737548.1, 163297, 163899, -, 603, 31420; cds-WP_021440903.1, NZ _KK737548.1, 164012, 164440, +, 429, 31342; cds-WP _032430022.1, NZ_KK737548.1, 164441, 165187, -, 747, 301235; cds-WP_021440902.1, NZ_KK737548.1, 165338, 166348, -, 1011, 207054; cds-WP_032428441.1, NZ_KK737548.1, 166397, 168478, -, 2082, 2207495; cds-WP_004181538.1, NZ_KK737548.1, 168484, 169173, -, 690, 509186; cds-WP _004150214.1, NZ_KK737548.1, 169178, 171298, -, 2121, 5641939; cds-WP_000135058.1, NZ_KK737548.1, 171317, 171592, -, 276, 1378005; cds-WP _002922664.1, NZ_KK737548.1, 171647, 172270, -, 624, 1924019; cds-WP _021440900.1, NZ _KK737548.1, 172529, 174205, +, 1677, 25246; cds-WP_002922654.1, NZ_KK737548.1, 174211, 174828, -, 618, 49808; cds-WP _021440899.1, NZ _KK737548.1, 175104, 176354, +, 1251, 22109; cds-WP_004151500.1, NZ_KK737548.1, 176410, 177468, -, 1059, 46080; cds-WP _023317368.1, NZ_KK737548.1, 177471, 178217, -, 747, 42202; cds-WP_002922636.1, NZ_KK737548.1, 178400, 179263, -, 864, 198597; cds-AF52_RS27235, NZ_KK737548.1, 184808, 184969, -, 162, 4768; cds-AF52_RS09865, NZ_KK737548.1, 185020, 185550, +, 531, 3877; cds-AF52_RS27245, NZ_KK737548.1, 185584, 185730, +, 147, 10526; cds-AF52_RS27250, NZ_KK737548.1, 189864, 189965, +, 102, 2559; cds-AF52_RS09860, NZ_KK737548.1, 190018, 190773, +, 756, 886; cds-WP_004216995.1, NZ_KK737548.1, 190811, 190942, -, 132, 678; cds-WP_187079195.1, NZ_KK737548.1, 190941, 191795, +, 855, 35248; cds-AF52_RS27260, NZ_KK737548.1, 191813, 191960, +, 148, 3539; cds-AF52_RS09845, NZ_KK737548.1, 195939, 196123, +, 185, 8068; cds-WP_032430029.1, NZ_KK737548.1, 196229, 197146, +, 918, 271640; cds-AF52_RS09835, NZ_KK737548.1, 197178, 197753, +, 576, 111506; cds-WP _013815099.1-49, NZ_KK737548.1, 197803, 198771, -, 969, 93686; cds-WP _032430030.1, NZ_KK737548.1, 198813, 199445, +, 633, 36; cds-WP _004181470.1, NZ_KK737548.1, 199673, 200161, +, 489, 17224; cds-WP_009484082.1, NZ_KK737548.1, 200629, 202374, -, 1746, 89768; cds-WP _004150073.1, NZ_KK737548.1, 202498, 202818, +, 321, 20775; cds-WP_021440835.1, NZ_KK737548.1, 202842, 203582, -, 741, 36397; cds-WP_004150074.1, NZ_KK737548.1, 203579, 204649, -, 1071,8653; cds-WP_004145129.1, NZ_KK737548.1, 204651, 205496, -, 846, 11179; cds-WP_014906831.1, NZ_KK737548.1, 205493, 206380, -, 888, 10136; cds-WP_004174008.1, NZ_KK737548.1, 206487, 207803, -, 1317, 37472; cds-WP_025861226.1, NZ_KK737548.1, 208076, 208444, -, 369, 30812; cds-WP_004174006.1, NZ_KK737548.1, 208441, 208662, -, 222, 14973; cds-WP _004145133.1, NZ_KK737548.1, 208827, 209540, -, 714, 31; cds-WP_002920803.1, NZ_KK737548.1, 209542, 210309, -, 768, 61889; cds-WP_004174005.1, NZ_KK737548.1, 210306, 211583, -, 1278, 7602; cds-WP_002920807.1, NZ_KK737548.1, 211580, 212506, -, 927, 17967; cds-WP _004150079.1, NZ_KK737548.1, 212568, 213677, -, 1110, 104314; cds-WP_021312829.1, NZ_KK737548.1, 214101, 214484, +, 384, 24972; cds-WP _002920812.1, NZ_KK737548.1, 214753, 215856, -, 1104, 279373; cds-WP _004150081.1, NZ_KK737548.1, 216114, 217358, -, 1245, 81023; cds-WP_021440838.1, NZ_KK737548.1, 217506, 218771, -, 1266, 11296; cds-WP _021440839.1, NZ_KK737548.1, 218890, 220413, +, 1524, 370909; cds-WP_032430034.1, NZ_KK737548.1, 220466, 221320, -, 855, 4546492; cds-WP_004173994.1, NZ_KK737548.1, 221590, 222645, -, 1056, 226502; cds-WP_002920817.1, NZ_KK737548.1, 222638, 223306, -, 669, 194274; cds-WP_004173992.1, NZ_KK737548.1, 223309, 224832, -, 1524, 1132043; cds-WP _002920820.1, NZ_KK737548.1, 225015, 225611, +, 597, 185808; cds-WP_002920822.1, NZ_KK737548.1, 225601, 225876, +, 276, 116734; cds-WP_004173989.1, NZ_KK737548.1, 225890, 226240, -, 351, 83293; cds-WP_004173987.1, NZ_KK737548.1, 226391, 227017, +, 627, 201186; cds-WP_004173985.1, NZ_KK737548.1, 227096, 229306, +, 2211, 230480; cds-WP_002920858.1, NZ_KK737548.1, 229410, 229655, -, 246, 65473; cds-WP _002920860.1, NZ_KK737548.1, 229862, 230527, +, 666, 1367312; cds-WP _002920861.1, NZ_KK737548.1, 230601, 231158, +, 558, 581903; cds-WP_004173981.1, NZ_KK737548.1, 231159, 232379, -, 1221, 45226; cds-WP_004145147.1, NZ_KK737548.1, 232513, 233562, +, 1050, 13201; cds-WP _002920863.1, NZ_KK737548.1, 233559, 233939, -, 381, 5001; cds-WP_002920864.1, NZ_KK737548.1, 233939, 234277, -, 339, 11231; cds-WP_004186016.1, NZ_KK737548.1, 234274, 235929, -, 1656, 144768; cds-WP _009308746.1, NZ_KK737548.1, 235929, 236831, -, 903, 32927; cds-WP _032430036.1, NZ_KK737548.1, 236828, 238813, -, 1986, 253367; cds-WP _002921035.1, NZ_KK737548.1, 238810, 239793, -, 984, 276970; cds-WP_002921037.1, NZ_KK737548.1, 239795, 240934, -, 1140, 80157; cds-WP _004181483.1, NZ_KK737548.1, 241331, 241837, -, 507, 87762; cds-WP _032430038.1, NZ_KK737548.1, 241935, 242795, +, 861, 1601; cds-WP_021440844.1, NZ_KK737548.1, 242934, 244502, +, 1569, 11730; cds-WP_004150102.1, NZ_KK737548.1, 244502, 245446, +, 945, 98; cds-WP_004151422.1, NZ_KK737548.1, 245443, 246276, +, 834, 4014; cds-WP_004173959.1, NZ_KK737548.1, 246276, 247040, +, 765, 35; cds-WP_009308750.1, NZ_KK737548.1, 247037, 247828, +, 792, 268; cds-WP_002921188.1, NZ_KK737548.1, 247816, 248214, +, 399, 29092; cds-WP_002921192.1, NZ_KK737548.1, 248552, 249565, +, 1014, 167751; cds-WP _004150109.1, NZ_KK737548.1, 249605, 250798, -, 1194, 241549; cds-WP_002921200.1, NZ_KK737548.1, 251028, 252524, +, 1497, 1416618; cds-WP _002921203.1, NZ_KK737548.1, 252580, 252915, -, 336, 35574; cds-WP _004145166.1, NZ_KK737548.1, 253298, 253735, +, 438, 792663; cds-WP_002921264.1, NZ_KK737548.1, 253992, 255464, +, 1473, 936575; cds-WP_004173956.1, NZ_KK737548.1, 255592, 256344, -, 753, 52247; cds-WP_004901172.1, NZ_KK737548.1, 256352, 258394, -, 2043, 1227223; cds-WP _021465315.1, NZ_KK737548.1, 258577, 259848, -, 1272, 2; cds-WP _021440845.1, NZ_KK737548.1, 260042, 260884, +, 843, 327335; cds-WP _002921271.1, NZ_KK737548.1, 260957, 262309, +, 1353, 833260; cds-WP_004173953.1, NZ_KK737548.1, 262363, 262986, -, 624, 76227; cds-WP _032430040.1, NZ_KK737548.1, 263113, 264261, +, 1149, 2259; cds-WP_004150117.1, NZ_KK737548.1, 264501, 266153, +, 1653, 196401; cds-WP_032430042.1, NZ_KK737548.1, 266220, 266753, +, 534, 6608; cds-WP _004211228.1, NZ_KK737548.1, 266788, 267546, -, 759, 113421; cds-WP_004145179.1, NZ_KK737548.1, 267666, 268565, +, 900, 89028; cds-WP_002921337.1, NZ_KK737548.1, 268672, 269703, +, 1032, 44526; cds-WP_004150118.1, NZ_KK737548.1, 269975, 271297, +, 1323, 21700; cds-WP _004901194.1, NZ_KK737548.1, 271335, 273386, -, 2052, 1283; cds-WP_002921434.1, NZ_KK737548.1, 273581, 274510, +, 930, 237778; cds-WP _032430046.1, NZ_KK737548.1, 274565, 276067, -, 1503, 828516; cds-WP _002921438.1, NZ_KK737548.1, 276287, 277573, -, 1287, 1479359; cds-WP_072198875.1, NZ_KK737548.1, 277730, 279760, -, 2031, 243566; cds-WP_032430051.1, NZ_KK737548.1, 279979, 283458, -, 3480, 49097; cds-WP _004173946.1, NZ_KK737548.1, 283440, 284549, -, 1110, 44189; cds-WP_072001583.1, NZ_KK737548.1, 284553, 286898, -, 2346, 60561; cds-WP_002921508.1, NZ_KK737548.1, 286909, 289527, -, 2619, 15404; cds-WP_002921510.1, NZ_KK737548.1, 289524, 290255, -, 732, 5525; cds-WP_002921513.1, NZ_KK737548.1, 290267, 290455, -, 189, 611; cds-WP _032430057.1, NZ_KK737548.1, 290624, 292177, +, 1554, 12600; cds-WP _002921518.1, NZ_KK737548.1, 292177, 292374, +, 198, 109; cds-WP_004188236.1, NZ_KK737548.1, 292371, 294050, +, 1680, 2777; cds-WP _032430059.1, NZ_KK737548.1, 294158, 295159, -, 1002, 152596; cds-WP _002921529.1, NZ_KK737548.1, 295171, 295647, -, 477, 7019; cds-WP _032430061.1, NZ_KK737548.1, 295647, 299699, -, 4053, 26039; cds-WP_004151435.1, NZ_KK737548.1, 299696, 302131, -, 2436, 52571; cds-WP_004173941.1, NZ_KK737548.1, 302128, 304239, -, 2112, 46217; cds-WP_004173940.1, NZ_KK737548.1, 304259, 305062, -, 804, 20892; cds-WP_004186070.1, NZ_KK737548.1, 305053, 305592, -, 540, 9813; cds-WP _004145206.1, NZ_KK737548.1, 305910, 306089, -, 180, 54; cds-WP _021440859.1, NZ_KK737548.1, 306108, 307121, -, 1014, 35415; cds-WP_021440860.1, NZ_KK737548.1, 307118, 308101, -, 984, 0; cds-WP_004145207.1, NZ_KK737548.1, 308112, 309014, -, 903, 0; cds-WP_002921786.1, NZ_KK737548.1, 309024, 310043, -, 1020, 22946; cds-WP _050484288.1, NZ_KK737548.1, 310178, 311779, -, 1602, 26610; cds-WP _013815099.1-50, NZ_KK737548.1, 311759, 312727, +, 969, 88916; cds-WP _004214574.1, NZ_KK737548.1, 313931, 315325, -, 1395, 3910; cds-WP_004152427.1, NZ_KK737548.1, 315517, 317190, -, 1674, 830388; cds-WP_002921907.1, NZ_KK737548.1, 317433, 318638, -, 1206, 356870; cds-WP _020803959.1, NZ_KK737548.1, 318880, 319461, -, 582, 3977; cds-WP_002921914.1, NZ_KK737548.1, 319667, 320248, +, 582, 147585; cds-WP _015959126.1, NZ_KK737548.1, 320226, 320666, +, 441, 85942; cds-WP _032428435.1, NZ_KK737548.1, 320635, 322965, -, 2331, 254740; cds-WP _002921917.1, NZ_KK737548.1, 323133, 323795, +, 663, 42398; cds-WP_002921918.1, NZ_KK737548.1, 323899, 324915, +, 1017, 34414; cds-WP_002921919.1, NZ_KK737548.1, 325027, 325776, +, 750, 54328; cds-WP_002921921.1, NZ_KK737548.1, 325780, 326715, +, 936, 20624; cds-WP_002921927.1, NZ_KK737548.1, 326820, 328100, +, 1281, 386171; cds-WP _021440865.1, NZ_KK737548.1, 328125, 329096, +, 972, 53634; cds-WP _002921929.1, NZ_KK737548.1, 329205, 329915, -, 711, 377913; cds-WP_021440866.1, NZ_KK737548.1, 330356, 331711, +, 1356, 247291; cds-WP_013815099.1-51, NZ_KK737548.1, 331921, 332889, -, 969, 87704; cds-AF52_RS26455, NZ_KK737548.1, 332922, 333029, -, 108, 9876; cds-WP _002922127.1, NZ_KK737548.1, 333330, 335399, -, 2070, 2150851; cds-WP_002922128.1, NZ_KK737548.1, 335409, 336320, -, 912, 1027908; cds-WP_002922129.1, NZ_KK737548.1, 336447, 336746, -, 300, 4834; cds-WP _004145225.1, NZ_KK737548.1, 336926, 337921, +, 996, 17672; cds-WP _004188180.1, NZ_KK737548.1, 337948, 338394, -, 447, 42779; cds-WP_002922133.1, NZ_KK737548.1, 338426, 338797, -, 372, 38554; cds-WP _004150156.1, NZ_KK737548.1, 338966, 340420, -, 1455, 6087; cds-WP_021440868.1, NZ_KK737548.1, 340513, 341835, -, 1323, 19430; cds-WP_014906789.1, NZ_KK737548.1, 342270, 343325, +, 1056, 271; cds-WP_002922346.1, NZ_KK737548.1, 343390, 344931, +, 1542, 3214; cds-WP_002922349.1, NZ_KK737548.1, 344909, 346090, +, 1182, 10093; cds-WP _002922351.1, NZ_KK737548.1, 346323, 347501, +, 1179, 117819; cds-WP_004188166.1, NZ_KK737548.1, 347540, 348358, -, 819, 241221; cds-WP_004173926.1, NZ_KK737548.1, 348673, 350706, +, 2034,64352; cds-WP_002922359.1, NZ_KK737548.1, 350875, 352131, +, 1257, 103958; cds-WP _021440872.1, NZ_KK737548.1, 352174, 352659, -, 486, 12175; cds-WP_021440873.1, NZ_KK737548.1, 352769, 353596, -, 828, 239916; cds-WP_021440874.1, NZ_KK737548.1, 353815, 354813, +, 999, 322918; cds-WP _021440875.1, NZ_KK737548.1, 354825, 355289, +, 465, 10305; cds-WP _004150167.1, NZ_KK737548.1, 355314, 356243, +, 930, 42242; cds-WP_032430066.1, NZ_KK737548.1, 356280, 357599, +, 1320, 39616; cds-WP _021440877.1, NZ_KK737548.1, 357678, 359183, +, 1506, 5209; cds-WP _004889285.1, NZ_KK737548.1, 359180, 359842, +, 663, 0; cds-WP_004889283.1, NZ_KK737548.1, 359835, 360695, +, 861, 0; cds-WP_021440878.1, NZ_KK737548.1, 360689, 361387, +, 699,
7158; cds-WP _032430069.1, NZ_KK737548.1, 361445, 363286, -, 1842, 34136; cds-WP _021440880.1, NZ_KK737548.1, 363283, 364671, -, 1389, 9492; cds-WP _004145238.1, NZ_KK737548.1, 364777, 365385, -, 609, 3860; cds-AF52_RS26460, NZ_KK737548.1, 365492, 365822, -, 331, 6477; cds-WP_002922397.1, NZ_KK737548.1, 366352, 368259, +, 1908, 428257; cds-WP_019705056.1, NZ_KK737548.1, 368360, 369508, +, 1149, 180708; cds-WP _002922401.1, NZ_KK737548.1, 369508, 370104, +, 597, 54715; cds-WP_002922402.1, NZ_KK737548.1, 370093, 370317, -, 225, 52144; cds-WP_002922410.1, NZ_KK737548.1, 370590, 370952, +, 363, 68281; cds-WP _002922413.1, NZ_KK737548.1, 371238, 372893, +, 1656, 7196470; cds-WP_002922415.1, NZ_KK737548.1, 372890, 373663, +, 774, 1777534; cds-WP _002922420.1, NZ_KK737548.1, 373663, 374847, +, 1185, 1174373; cds-WP _002922423.1, NZ_KK737548.1, 374940, 375413, +, 474, 274342; cds-WP _004164645.1, NZ_KK737548.1, 375490, 375757, +, 268, 33251; cds-WP _032430073.1, NZ_KK737548.1, 376056, 376773, +, 718, 66143; cds-WP _004185922.1, NZ_KK737548.1, 376896, 377726, -, 831, 2225266; cds-WP_002920136.1, NZ_KK737548.1, 377949, 378167, +, 219, 41284; cds-WP_002920140.1, NZ_KK737548.1, 378226, 378813, -, 588, 546102; cds-WP_002920143.1, NZ_KK737548.1, 378907, 379107, -, 201, 20632; cds-WP_004150031.1, NZ_KK737548.1, 379119, 380924, -, 1806, 293931; cds-WP _004151401.1, NZ_KK737548.1, 380911, 381474, -, 564, 230938; cds-WP _032430077.1, NZ_KK737548.1, 381648, 383552, +, 1905, 386389; cds-WP _072145335.1, NZ_KK737548.1, 383780, 384778, +, 999, 171825; cds-WP _002920151.1, NZ_KK737548.1, 384775, 384993, +, 219, 102817; cds-WP _021440811.1, NZ_KK737548.1, 385030, 385899, +, 870, 401350; cds-WP_002920158.1, NZ_KK737548.1, 385954, 386358, -, 405, 247860; cds-WP_000242758.1, NZ_KK737548.1, 386665, 387297, +, 633, 2469155; cds-WP _004213494.1, NZ_KK737548.1, 387349, 389427, +, 2079, 482463; cds-WP _021440813.1, NZ_KK737548.1, 389417, 390637, -, 1221, 72376; cds-WP_021440814.1, NZ_KK737548.1, 390728, 391291, -, 564, 7791; cds-WP_004181446.1, NZ_KK737548.1, 391324, 391926, -, 603, 4817; cds-WP _002920231.1, NZ_KK737548.1, 391916, 392083, -, 168, 159337; cds-WP_002920238.1, NZ_KK737548.1, 392190, 392759, -, 570, 517303; cds-WP_002920240.1, NZ_KK737548.1, 393036, 394223, +, 1188, 315997; cds-WP_004174045.1, NZ_KK737548.1, 394220, 395542, -, 1323, 241006; cds-WP _002920243.1, NZ_KK737548.1, 395781, 398324, +, 2544, 368; cds-WP_002920249.1, NZ_KK737548.1, 398321, 398647, +, 327, 56; cds-WP _002920252.1, NZ_KK737548.1, 398801, 400174, +, 1374, 21172; cds-WP _002920253.1, NZ_KK737548.1, 400247, 401251, -, 1005, 1628472; cds-WP _004188345.1, NZ_KK737548.1, 401244, 402005, -, 762, 742145; cds-WP_002920259.1, NZ_KK737548.1, 401998, 402675, -, 678, 978307; cds-WP_002920260.1, NZ_KK737548.1, 402693, 403520, -, 828, 2102251; cds-WP _021440816.1, NZ_KK737548.1, 403599, 404885, -, 1287, 2961174; cds-WP _004150047.1, NZ_KK737548.1, 404970, 406064, -, 1095, 1976832; cds-WP _002920267.1, NZ_KK737548.1, 406120, 406641, -, 522, 879644; cds-WP _021440817.1, NZ_KK737548.1, 406912, 408156, -, 1245, 20266; cds-WP _002920279.1, NZ_KK737548.1, 408089, 408472, -, 384, 11520; cds-WP_004174029.1, NZ_KK737548.1, 408462, 408890, -, 429, 400; cds-WP _032428423.1, NZ_KK737548.1, 408880, 409413, -, 534, 337; cds-WP _004174027.1, NZ_KK737548.1, 409397, 410197, -, 801, 429; cds-WP_004185951.1, NZ_KK737548.1, 410316, 412874, +, 2559, 799955; cds-WP _032430083.1, NZ_KK737548.1, 412928, 413488, -, 561, 84082; cds-WP_032430086.1, NZ_KK737548.1, 413809, 415938, +, 2130, 530286; cds-WP_004181456.1, NZ_KK737548.1, 415999, 416682, +, 684, 571928; cds-WP _002920326.1, NZ_KK737548.1, 416679, 417080, +, 402, 633277; cds-WP_004188324.1, NZ_KK737548.1, 417099, 417983, +, 885, 413361; cds-WP_032430089.1, NZ_KK737548.1, 418344, 419966, +, 1623, 3536828; cds-WP_002920333.1, NZ_KK737548.1, 420044, 421399, -, 1356, 399017; cds-WP_001157751.1, NZ_KK737548.1, 421396, 422115, -, 720, 503347; cds-WP_002920503.1, NZ_KK737548.1, 422343, 422816, +, 474, 197114; cds-WP_004150056.1, NZ_KK737548.1, 422917, 425247, +, 2331, 1729992; cds-WP _013815099.1-52, NZ_KK737548.1, 425442, 426410, -, 969, 74729; cds-WP_002920506.1, NZ_KK737548.1, 426726, 426953, +, 228, 58960; cds-WP_014906839.1, NZ_KK737548.1, 426982, 429300, +, 2319, 192491; cds-WP_002920509.1, NZ_KK737548.1, 429310, 429549, +, 240, 6893; cds-WP _002920510.1, NZ_KK737548.1, 429638, 429907, -, 270, 161209; cds-WP_032430095.1, NZ_KK737548.1, 430028, 430801, -, 774, 16324; cds-WP_004185961.1, NZ_KK737548.1, 430840, 431514, +, 675, 32055; cds-WP _002920540.1, NZ_KK737548.1, 431573, 432148, +, 576, 526982; cds-WP _002920542.1, NZ_KK737548.1, 432485, 433801, +, 1317, 50162; cds-WP_004185965.1, NZ_KK737548.1, 433904, 435997, -, 2094, 978790; cds-WP_004151407.1, NZ_KK737548.1, 436008, 438398, -, 2391, 2717367; cds-WP _002920545.1, NZ_KK737548.1, 439023, 441728, +, 2706, 2268244; cds-WP_002920548.1, NZ_KK737548.1, 441744, 442502, -, 759, 455361; cds-WP _032428425.1, NZ_KK737548.1, 442552, 443382, -, 831, 407433; cds-WP _002920552.1, NZ_KK737548.1, 443433, 443762, -, 330, 145325; cds-WP_002920554.1, NZ_KK737548.1, 443967, 445475, +, 1509, 5219154; cds-WP _004174017.1, NZ_KK737548.1, 445538, 445837, -, 300, 404090; cds-WP _016529833.1, NZ_KK737548.1, 445837, 446184, -, 348, 100822; cds-WP _002920561.1, NZ_KK737548.1, 446360, 448807, -, 2448, 601319; cds-WP_002920564.1, NZ_KK737548.1, 448825, 450258, -, 1434, 100206; cds-WP _002920566.1, NZ_KK737548.1, 450258, 451553, -, 1296, 542379; cds-WP _023342583.1, NZ_KK737548.1, 451634, 453610, -, 1977, 132790; cds-WP_004150069.1, NZ_KK737548.1, 453607, 455793, -, 2187, 667729; cds-WP_002920570.1, NZ_KK737548.1, 456031, 457137, -, 1107, 293797; cds-WP_004151411.1, NZ_KK737548.1, 457241, 457603, +, 363, 50; cds-WP_002920773.1, NZ_KK737548.1, 457600, 458940, -, 1341, 32205; cds-WP_002920775.1, NZ_KK737548.1, 458940, 459470, -, 531, 37835; cds-WP _002920777.1, NZ_KK737548.1, 459651, 460646, -, 996, 90666; cds-WP_002920779.1, NZ_KK737548.1, 460760, 461455, -, 696, 102889; cds-WP _021440831.1, NZ_KK737548.1, 461577, 462614, -, 1038, 247677; cds-WP _013815099.1-53, NZ_KK737548.1, 462956, 463924, -, 969, 74280; cds-AF52_RS08695, NZ_KK737548.1, 463958, 464491, +, 534, 6821; cds-WP_002922602.1, NZ_KK737548.1, 464588, 465304, +, 717, 610162; cds-WP _004173904.1, NZ_KK737548.1, 465415, 466056, +, 642, 219995; cds-WP _004173905.1, NZ_KK737548.1, 466092, 467108, -, 1017, 666450; cds-WP_002922598.1, NZ_KK737548.1, 467284, 467880, -, 597, 181712; cds-WP _004145270.1, NZ_KK737548.1, 468005, 468463, -, 459, 232596; cds-WP _004188117.1, NZ_KK737548.1, 468441, 469655, -, 1215, 377680; cds-WP_002922589.1, NZ_KK737548.1, 469828, 470493, +, 666, 174826; cds-WP_000091955.1, NZ_KK737548.1, 470710, 470946, +, 237, 1494509; cds-WP _002922510.1, NZ_KK737548.1, 470967, 471134, +, 168, 1094159; cds-WP_004173907.1, NZ_KK737548.1, 471270, 472079, +, 810, 35285; cds-WP_002922501.1, NZ_KK737548.1, 472268, 472747, -, 480, 269868; cds-WP _004145264.1, NZ_KK737548.1, 472744, 473520, -, 777, 575687; cds-WP _004145263.1, NZ_KK737548.1, 473520, 474794, -, 1275, 682296; cds-WP_002922498.1, NZ_KK737548.1, 474886, 475875, -, 990, 615731; cds-WP_032430101.1, NZ_KK737548.1, 475905, 476999, -, 1095, 549355; cds-WP _009308781.1, NZ_KK737548.1, 477002, 478129, -, 1128, 185272; cds-WP_021440889.1, NZ_KK737548.1, 478126, 479202, -, 1077, 321300; cds-WP_021440888.1, NZ_KK737548.1, 479314, 480276, +, 963, 96309; cds-WP _032428440.1, NZ_KK737548.1, 480295, 481374, +, 1080, 375531; cds-WP_032430104.1, NZ_KK737548.1, 481418, 482587, -, 1170, 429916; cds-WP_002922468.1, NZ_KK737548.1, 482565, 483473, -, 909, 743593; cds-WP _004186128.1, NZ_KK737548.1, 483473, 484444, -, 972, 234360; cds-WP _002922465.1, NZ_KK737548.1, 484448, 485506, -, 1059, 971595; cds-WP _002922463.1, NZ_KK737548.1, 485516, 486448, -, 933, 908721; cds-WP_002922462.1, NZ_KK737548.1, 486662, 487855, +, 1194, 1370002; cds-WP_002922461.1, NZ_KK737548.1, 487868, 488893, +, 1026, 1895422; cds-WP_032428438.1, NZ_KK737548.1, 489062, 489850, +, 789, 597524; cds-WP_002922459.1, NZ_KK737548.1, 489856, 490803, -, 948, 296894; cds-WP_032428437.1, NZ_KK737548.1, 490807, 492078, -, 1272, 232473; cds-WP _004152046.1, NZ_KK737548.1, 492088, 493632, -, 1545, 242983; cds-WP_002922436.1, NZ_KK737548.1, 493878, 494309, +, 432, 931506; cds-WP_002922429.1, NZ_KK737548.1, 494416, 494667, +, 252, 1132760; cds-WP_002922428.1, NZ_KK737548.1, 494728, 495195, +, 468, 1894414; cds-WP _002922427.1, NZ_KK737548.1, 495195, 496214, +, 1020, 1533632; cds-WP_002922426.1, NZ_KK737548.1, 496288, 497109, +, 822, 373563; cds-AF52_RS27300, NZ_KK737548.1, 497212, 497488, -, 277, 18761; cds-WP _032430113.1, NZ_KK737548.1, 498141, 498462, -, 322, 12112; cds-WP_002920130.1, NZ_KK737548.1, 498606, 499328, +, 723, 419387; cds-WP_002920128.1, NZ_KK737548.1, 499328, 499714, +, 387, 28701; cds-WP _004889490.1, NZ_KK737548.1, 499714, 500073, +, 360, 48653; cds-WP_002920119.1, NZ_KK737548.1, 500081, 500368, +, 288, 98470; cds-WP_002920115.1, NZ_KK737548.1, 500493, 500867, +, 375, 7784182; cds-WP_002920113.1, NZ_KK737548.1, 500964, 501434, +, 471, 16601278; cds-WP _002920103.1, NZ_KK737548.1, 501531, 503645, +, 2115, 89066909; cds-WP _004174069.1, NZ_KK737548.1, 503715, 504899, +, 1185, 85971667; cds-WP _004152415.1, NZ_KK737548.1, 505080, 505274, +, 195, 36146; cds-WP_002919971.1, NZ_KK737548.1, 505348, 505824, +, 477, 88812; cds-WP_020801891.1, NZ_KK737548.1, 505808, 506293, -, 486, 14222; cds-WP_001181005.1, NZ_KK737548.1, 506677, 506988, +, 312, 13923212; cds-WP_002919796.1, NZ_KK737548.1, 507021, 507650, +, 630, 23488641; cds-WP _002919794.1, NZ_KK737548.1, 507661, 508266, +, 606, 23551825; cds-WP _000617546.1, NZ_KK737548.1, 508263, 508565, +, 303, 11695539; cds-WP_002919786.1, NZ_KK737548.1, 508583, 509404, +, 822, 38043802; cds-WP _001138115.1, NZ_KK737548.1, 509421, 509699, +, 279, 16401637; cds-WP_002919773.1, NZ_KK737548.1, 509714, 510046, +, 333, 16496081; cds-WP_002919766.1, NZ_KK737548.1, 510064, 510762, +, 699, 29338149; cds-WP_002919759.1, NZ_KK737548.1, 510775, 511185, +, 411, 14820490; cds-WP _002919754.1, NZ_KK737548.1, 511185, 511376, +, 192, 6056967; cds-WP_002919751.1, NZ_KK737548.1, 511376, 511630, +, 255, 5197239; cds-WP _002919748.1, NZ_KK737548.1, 511796, 512167, +, 372, 9325206; cds-WP_000729185.1, NZ_KK737548.1, 512178, 512492, +, 315, 13302073; cds-WP _001096200.1, NZ_KK737548.1, 512507, 513046, +, 540, 27987517; cds-WP_002919667.1, NZ_KK737548.1, 513061, 513366, +, 306, 20541713; cds-WP_002919665.1, NZ_KK737548.1, 513400, 513792, +, 393, 13759116; cds-WP _002919662.1, NZ_KK737548.1, 513805, 514338, +, 534, 20426559; cds-WP _000358960.1, NZ_KK737548.1, 514348, 514701, +, 354, 17910734; cds-WP_002919545.1, NZ_KK737548.1, 514716, 515219, +, 504, 18203191; cds-WP _001140434.1, NZ_KK737548.1, 515223, 515402, +, 180, 6094160; cds-WP _002919516.1, NZ_KK737548.1, 515406, 515840, +, 435, 23514844; cds-WP_002919515.1, NZ_KK737548.1, 515848, 517179, +, 1332, 43372031; cds-WP _000868187.1, NZ_KK737548.1, 517213, 517329, +, 117, 3506399; cds-WP _002919259.1, NZ_KK737548.1, 517476, 517832, +, 357, 13915840; cds-WP _002919257.1, NZ_KK737548.1, 517849, 518238, +, 390, 20066577; cds-WP_002919224.1, NZ_KK737548.1, 518272, 518892, +, 621, 22477649; cds-WP _002919219.1, NZ_KK737548.1, 518918, 519907, +, 990, 45116302; cds-WP_002919206.1, NZ_KK737548.1, 519948, 520334, +, 387, 10630712; cds-WP_002919204.1, NZ_KK737548.1, 520443, 520811, +, 369, 32303; cds-WP_004185894.1, NZ_KK737548.1, 520814, 521239, +, 426, 37238; cds-WP _004145324.1, NZ_KK737548.1, 521297, 521575, +, 279, 56176; cds-WP_004145325.1, NZ_KK737548.1, 521511, 521924, -, 414, 131535; cds-WP_032430127.1, NZ_KK737548.1, 522065, 523441, -, 1377, 698494; cds-WP_004212524.1, NZ_KK737548.1, 523455, 524750, -, 1296, 515197; cds-WP _032430129.1, NZ_KK737548.1, 524803, 525750, -, 948, 994930; cds-WP_004889527.1, NZ_KK737548.1, 525765, 526274, -, 510, 940649; cds-WP_032428422.1, NZ_KK737548.1, 526403, 527527, +, 1125, 67815; cds-WP _002919137.1, NZ_KK737548.1, 527499, 527972, +, 474, 518432; cds-WP_004145330.1, NZ_KK737548.1, 527999, 528541, +, 543, 737861; cds-WP_021440795.1, NZ_KK737548.1, 528546, 529118, +, 573, 508410; cds-WP_002919126.1, NZ_KK737548.1, 529122, 529940, +, 819, 931050; cds-WP _002919125.1, NZ_KK737548.1, 529937, 530194, +, 258, 37634; cds-
AF52_RS08260, NZ_KK737548.1, 530170, 530502, -, 333, 75251; cds-AF52_RS08255, NZ_KK737548.1, 530559, 530709, -, 151, 12731; cds-AF52_RS08250, NZ_KK737548.1, 538177, 538410, -, 234, 3591; cds-WP_002919103.1, NZ_KK737548.1, 546983, 547204, -, 222, 65420; cds-WP _032430131.1, NZ_KK737548.1, 547497, 550607, -, 3111, 529916; cds-WP_002919101.1, NZ_KK737548.1, 550620, 551759, -, 1140, 447; cds-WP _021440791.1, NZ_KK737548.1, 552138, 552791, +, 654, 125; cds-WP _000462905.1, NZ_KK737548.1, 552876, 553172, -, 297, 557070; cds-WP _004144972.1, NZ_KK737548.1, 553197, 554162, -, 966, 1916724; cds-WP_004144971.1, NZ_KK737548.1, 554520, 555401, -, 882, 345561; cds-WP_004188394.1, NZ_KK737548.1, 555413, 556864, -, 1452, 183432; cds-WP _002918740.1, NZ_KK737548.1, 556854, 557096, -, 243, 20177; cds-WP _002918738.1, NZ_KK737548.1, 557207, 558556, -, 1350, 2170589; cds-WP _002918736.1, NZ_KK737548.1, 558567, 559034, -, 468, 959810; cds-WP_019725248.1, NZ_KK737548.1, 559057, 559509, -, 453, 769097; cds-WP _002918689.1, NZ_KK737548.1, 559733, 560341, -, 609, 55157; cds-WP_004211677.1, NZ_KK737548.1, 560341, 561342, -, 1002, 40800; cds-WP_004211674.1, NZ_KK737548.1, 561572, 561763, +, 192, 718; cds-WP_002918686.1, NZ_KK737548.1, 561843, 563783, +, 1941, 242023; cds-WP _002918653.1, NZ_KK737548.1, 564089, 565132, +, 1044, 1537883; cds-WP_004149974.1, NZ_KK737548.1, 565203, 566195, +, 993, 1126220; cds-WP _002918648.1, NZ_KK737548.1, 566195, 566683, +, 489, 218079; cds-WP_032430132.1, NZ_KK737548.1, 566691, 567272, +, 582, 292261; cds-WP _002918644.1, NZ_KK737548.1, 567275, 568744, +, 1470, 848513; cds-WP_032417624.1, NZ_KK737548.1, 568782, 572579, +, 3798, 471741; cds-WP _004144958.1, NZ_KK737548.1, 572668, 574113, +, 1446, 235187; cds-WP _002918641.1, NZ_KK737548.1, 574149, 575078, -, 930, 52780; cds-WP_002918640.1, NZ_KK737548.1, 575210, 575413, +, 204, 11585; cds-WP_002918639.1, NZ_KK737548.1, 575421, 576353, +, 933, 4699; cds-WP_002918632.1, NZ_KK737548.1, 576359, 578326, +, 1968, 31318; cds-WP_002918629.1, NZ_KK737548.1, 578406, 578681, +, 276, 3111; cds-WP_002918627.1, NZ_KK737548.1, 578732, 578998, -, 267, 67237; cds-WP _032428421.1, NZ_KK737548.1, 579097, 579360, -, 264, 56015; cds-WP_002918625.1, NZ_KK737548.1, 579735, 580205, -, 471, 42796; cds-WP_002918570.1, NZ_KK737548.1, 580620, 581558, +, 939, 507488; cds-WP _002918568.1, NZ_KK737548.1, 581695, 582753, -, 1059, 104206; cds-WP_032430134.1, NZ_KK737548.1, 582841, 584208, -, 1368, 443774; cds-WP_002918565.1, NZ_KK737548.1, 584382, 584780, -, 399, 224565; cds-WP _004900926.1, NZ_KK737548.1, 584971, 586098, +, 1128, 454372; cds-WP _002918559.1, NZ_KK737548.1, 586364, 586792, +, 429, 10587461; cds-WP _000829818.1, NZ_KK737548.1, 586808, 587200, +, 393, 17960410; cds-WP_002918467.1, NZ_KK737548.1, 587512, 588150, +, 639, 2829176; cds-WP_002918465.1, NZ_KK737548.1, 588154, 588648, +, 495, 355647; cds-WP_004185859.1, NZ_KK737548.1, 588773, 589477, +, 705, 62543; cds-WP _002918461.1, NZ_KK737548.1, 589532, 590950, -, 1419, 89470; cds-WP _004144939.1, NZ_KK737548.1, 590960, 595420, -, 4461, 1428391; cds-WP _002918455.1, NZ_KK737548.1, 596094, 597026, +, 933, 2; cds-AF52_RS08010, NZ_KK737548.1, 597078, 597653, -, 576, 442; cds-AF52_RS08005, NZ_KK737548.1, 597707, 597899, -, 193, 6221; cds-WP _032430135.1, NZ_KK737548.1, 600002, 601147, -, 1146, 1042218; cds-WP _002917952.1, NZ_KK737548.1, 601212, 602102, -, 891, 1395156; cds-WP _002917954.1, NZ_KK737548.1, 602129, 602899, -, 771, 734559; cds-WP_020802049.1, NZ_KK737548.1, 602926, 604257, -, 1332, 1178567; cds-WP _002917960.1, NZ_KK737548.1, 604650, 606221, +, 1572, 886123; cds-WP_004144878.1, NZ_KK737548.1, 606434, 607297, -, 864, 129056; cds-WP_032430136.1, NZ_KK737548.1, 607361, 609469, +, 2109, 492859; cds-WP _002918206.1, NZ_KK737548.1, 609427, 609813, +, 387, 21760; cds-WP _002918211.1, NZ_KK737548.1, 609839, 610429, +, 591, 213889; cds-WP_002918214.1, NZ_KK737548.1, 610439, 611014, +, 576, 177089; cds-WP_004149921.1, NZ_KK737548.1, 611136, 612176, -, 1041, 22802; cds-WP _002918218.1, NZ_KK737548.1, 612252, 612899, -, 648, 31769; cds-WP_002918221.1, NZ_KK737548.1, 613028, 613564, +, 537, 11061; cds-WP_002918223.1, NZ_KK737548.1, 613526, 613969, -, 444, 45042; cds-WP_002918226.1, NZ_KK737548.1, 614019, 614309, +, 291,66975; cds-WP_002918229.1, NZ_KK737548.1, 614296, 614799, -, 504, 82351; cds-WP_002918231.1, NZ_KK737548.1, 614793, 615317, -, 525, 108463; cds-WP_002918232.1, NZ_KK737548.1, 615539, 616534, +, 996, 81; cds-WP_020802035.1, NZ_KK737548.1, 616540, 617418, +, 879, 20; cds-WP_020802054.1, NZ_KK737548.1, 617562, 618569, +, 1008, 231425; cds-WP _002918235.1, NZ_KK737548.1, 618614, 619858, -, 1245, 461556; cds-WP_004150943.1, NZ_KK737548.1, 620003, 621934, -, 1932, 2899246; cds-WP _085903200.1, NZ_KK737548.1, 621927, 622007, -, 81, 512584; cds-WP_002918241.1, NZ_KK737548.1, 622112, 622996, -, 885, 7436332; cds-WP _004150944.1, NZ_KK737548.1, 623105, 625240, -, 2136, 9234085; cds-WP_002918244.1, NZ_KK737548.1, 625483, 625752, -, 270, 809875; cds-WP _023280600.1, NZ_KK737548.1, 625901, 626845, -, 945, 280166; cds-WP _002918248.1, NZ_KK737548.1, 626845, 627246, -, 402, 286021; cds-WP_002918250.1, NZ_KK737548.1, 627349, 630039, -, 2691, 8538168; cds-WP_002918252.1, NZ_KK737548.1, 630064, 631551, -, 1488, 2985235; cds-WP_002918364.1, NZ_KK737548.1, 631579, 632031, -, 453, 1483274; cds-WP _004144895.1, NZ_KK737548.1, 632624, 633967, +, 1344, 349178; cds-WP_013815099.1-54, NZ_KK737548.1, 634121, 635089, -, 969, 76858; cds-AF52_RS0124215, NZ_KK737548.1, 635123, 635506, +, 384, 22118; cds-WP _004174150.1, NZ_KK737548.1, 635644, 636273, +, 630, 5359; cds-WP_002918367.1, NZ_KK737548.1, 636616, 637167, +, 552, 12850; cds-WP _014906872.1, NZ_KK737548.1, 637209, 637457, -, 249, 7601; cds-WP_002918369.1, NZ_KK737548.1, 637851, 638180, -, 330, 1150903; cds-WP _002918370.1, NZ_KK737548.1, 638410, 639747, -, 1338, 338049; cds-WP_002918371.1, NZ_KK737548.1, 639740, 640588, -, 849, 917950; cds-WP_002918372.1, NZ_KK737548.1, 640682, 642616, -, 1935, 6966241; cds-WP _002918373.1, NZ_KK737548.1, 642716, 643345, -, 630, 1898481; cds-WP_002918374.1, NZ_KK737548.1, 643469, 643762, +, 294, 777412; cds-WP_002918375.1, NZ_KK737548.1, 643915, 644391, -, 477, 368896; cds-WP_004149937.1, NZ_KK737548.1, 644627, 646060, +, 1434, 434926; cds-WP_002918377.1, NZ_KK737548.1, 646112, 647290, -, 1179, 1372336; cds-WP_004149938.1, NZ_KK737548.1, 647306, 648271, -, 966, 1411901; cds-WP _002434222.1, NZ_KK737548.1, 648365, 648622, -, 258, 4211150; cds-WP _002918379.1, NZ_KK737548.1, 648643, 648954, -, 312, 5488232; cds-WP_004144907.1, NZ_KK737548.1, 649215, 650186, +, 972, 522255; cds-WP_002918381.1, NZ_KK737548.1, 650383, 650655, +, 273, 40785; cds-WP_002918382.1, NZ_KK737548.1, 650711, 651970, -, 1260, 1087431; cds-WP 004144910.1, NZ_KK737548.1, 652024, 652278, -, 255, 222976; cds-WP_004174142.1, NZ_KK737548.1, 652415, 652705, -, 291, 118949; cds-WP_032430139.1, NZ_KK737548.1, 652705, 653340, -, 636, 1017239; cds-WP _002918387.1, NZ_KK737548.1, 653359, 653910, -, 552, 480820; cds-WP_004150949.1, NZ_KK737548.1, 653915, 654697, -, 783, 718536; cds-WP _004150950.1, NZ_KK737548.1, 654705, 655517, -, 813, 404117; cds-WP_004149949.1, NZ_KK737548.1, 655727, 656704, +, 978, 123967; cds-WP_002918399.1, NZ_KK737548.1, 656719, 657705, +, 987, 314470; cds-WP_002918405.1, NZ_KK737548.1, 657720, 658286, +, 567, 176349; cds-WP _002918413.1, NZ_KK737548.1, 658283, 658858, +, 576, 855942; cds-WP _002918415.1, NZ_KK737548.1, 658827, 659372, +, 546, 302911; cds-WP_002918417.1, NZ_KK737548.1, 659379, 660104, +, 726, 891765; cds-WP_002918420.1, NZ_KK737548.1, 660152, 661585, +, 1434, 1574281; cds-WP_002918423.1, NZ_KK737548.1, 661608, 661895, +, 288, 452096; cds-WP_004144926.1, NZ_KK737548.1, 661966, 662454, +, 489, 613726; cds-WP_002918428.1, NZ_KK737548.1, 662500, 663354, +, 855, 916140; cds-WP _002918431.1, NZ_KK737548.1, 663351, 663623, +, 273, 202731; cds-WP _032430141.1, NZ_KK737548.1, 663687, 664412, -, 726, 294669; cds-WP_004889584.1, NZ_KK737548.1, 664409, 665062, -, 654, 277973; cds-WP _002918444.1, NZ_KK737548.1, 665296, 667635, -, 2340, 885159; cds-WP_021440775.1, NZ_KK737548.1, 667782, 668846, -, 1065, 885; cds-WP _032428420.1, NZ_KK737548.1, 669143, 670159, +, 1017, 986; cds-AF52_RS07625, NZ_KK737548.1, 670245, 670964, +, 720, 2; cds-WP_032430142.1, NZ_KK737548.1, 670997, 671650, +, 654, 40137; cds-WP_032175380.1, NZ_KK737548.1, 671925, 672273, +, 349, 33713; cds-AF52_RS07610, NZ_KK737548.1, 672318, 672869, -, 552, 6017; cds-WP_004144865.1, NZ_KK737548.1, 672974, 673870, +, 897, 167113; cds-WP_002917923.1, NZ_KK737548.1, 673912, 674280, -, 369, 221403; cds-WP _002917922.1, NZ_KK737548.1, 674407, 675393, -, 987, 57725; cds-WP _002917920.1, NZ_KK737548.1, 675474, 675866, -, 393, 45; cds-WP_004181402.1, NZ_KK737548.1, 676212, 676508, -, 297, 19256; cds-WP_002917917.1, NZ_KK737548.1, 676505, 676903, -, 399, 306949; cds-WP_002917916.1, NZ_KK737548.1, 676906, 677211, -, 306, 337574; cds-WP _002917914.1, NZ_KK737548.1, 677257, 677625, -, 369, 800021; cds-WP_004218213.1, NZ_KK737548.1, 677775, 678152, -, 378, 165924; cds-WP _002917909.1, NZ_KK737548.1, 678161, 678823, -, 663, 373196; cds-WP _002917907.1, NZ_KK737548.1, 679164, 679940, -, 777, 236304; cds-WP_002917905.1, NZ_KK737548.1, 680068, 681369, -, 1302, 46145; cds-WP _050484286.1, NZ_KK737548.1, 681850, 683202, +, 1353, 417918; cds-WP_002917898.1, NZ_KK737548.1, 683281, 684768, +, 1488, 19925; cds-WP _002917897.1, NZ_KK737548.1, 684898, 685452, +, 555, 52195; cds-WP_002917895.1, NZ_KK737548.1, 685468, 686715, -, 1248, 519398; cds-WP _004185787.1, NZ_KK737548.1, 686982, 687953, -, 972, 208496; cds-WP _004181399.1, NZ_KK737548.1, 688225, 689214, -, 990, 1188918; cds-WP_004144843.1, NZ_KK737548.1, 689288, 689788, -, 501, 25821; cds-WP _021440752.1, NZ_KK737548.1, 689808, 690410, -, 603, 145293; cds-WP_002917889.1, NZ_KK737548.1, 690454, 691161, +, 708, 4627; cds-WP_021440751.1, NZ_KK737548.1, 691247, 692377, +, 1131, 193114; cds-WP_004900808.1, NZ_KK737548.1, 692486, 694507, -, 2022, 614033; cds-WP _019704938.1, NZ_KK737548.1, 694692, 696290, -, 1599, 34871; cds-WP _004144837.1, NZ_KK737548.1, 696328, 697299, -, 972, 25207; cds-WP_022615670.1, NZ_KK737548.1, 697518, 699005, +, 1488, 3593; cds-WP_004174225.1, NZ_KK737548.1, 699002, 700036, +, 1035, 5473; cds-WP _032430144.1, NZ_KK737548.1, 700037, 701035, +, 999, 8; cds-WP _032430145.1, NZ_KK737548.1, 701037, 702038, +, 1002, 0; cds-WP _002917724.1, NZ_KK737548.1, 702050, 702937, +, 888, 17; cds-WP _002917723.1, NZ_KK737548.1, 702934, 703230, +, 297, 172; cds-WP_023316052.1, NZ_KK737548.1, 703277, 704656, -, 1380, 106433; cds-WP_002917718.1, NZ_KK737548.1, 704914, 705468, -, 555, 27060; cds-WP _021440748.1, NZ_KK737548.1, 705706, 706482, +, 777, 14276; cds-WP_004174228.1, NZ_KK737548.1, 706638, 708287, -, 1650, 102247; cds-WP _004174229.1, NZ_KK737548.1, 708363, 709784, -, 1422, 49574; cds-WP _004181378.1, NZ_KK737548.1, 709795, 710427, -, 633, 218082; cds-WP _002917682.1, NZ_KK737548.1, 710438, 711508, -, 1071, 194797; cds-WP_020324489.1, NZ_KK737548.1, 711664, 711849, -, 186, 285; cds-WP_002917681.1, NZ_KK737548.1, 712119, 713216, +, 1098, 58050; cds-WP _002917680.1, NZ_KK737548.1, 713318, 715243, +, 1926, 171682; cds-WP_002917679.1, NZ_KK737548.1, 715221, 715751, -, 531, 3181; cds-WP _021440747.1, NZ_KK737548.1, 715752, 716105, -, 354, 0; cds-WP _032430191.1, NZ_KK737548.1, 716126, 717277, -, 1152, 84; cds-WP _013815099.1-55, NZ_KK737548.1, 717293, 718261, +, 969, 83005; cds-WP_015875176.1, NZ_KK737548.1, 718377, 718805, -, 429, 0; cds-WP_002917676.1, NZ_KK737548.1, 719219, 720886, +, 1668, 0; cds-WP _021440746.1, NZ_KK737548.1, 720899, 721483, +, 585, 199; cds-WP_002917670.1, NZ_KK737548.1, 721486, 721911, +, 426, 0; cds-AF52_RS0124235, NZ_KK737548.1, 721924, 723747, +, 1824, 613; cds-WP_015959037.1, NZ_KK737548.1, 723801, 724604, +, 804, 2069; cds-WP_002917655.1, NZ_KK737548.1, 724656, 724961, -, 306, 6316; cds-WP _002917651.1, NZ_KK737548.1, 724988, 725563, -, 576, 6938; cds-WP_004144810.1, NZ_KK737548.1, 725804, 726466, +, 663, 92; cds-AF52_RS07345, NZ_KK737548.1, 726557, 728848, -, 2292, 516; cds-WP_013815099.1-56, NZ_KK737548.1, 728883, 729851, +, 969, 86529; cds-AF52_RS07335, NZ_KK737548.1, 729909, 730865, -, 957, 4037; cds-WP _004174237.1, NZ_KK737548.1, 730870, 731484, -, 615, 25791; cds-WP_021440741.1, NZ_KK737548.1,
731785, 732150, +, 366, 0; cds-WP _023313669.1, NZ_KK737548.1, 732219, 732491, -, 273, 2; cds-AF52_RS27330, NZ_KK737548.1, 732623, 732801, +, 179, 10056; cds-AF52_RS27335, NZ_KK737548.1, 735673, 735777, -, 105, 402; cds-WP_004152286.1, NZ_KK737548.1, 736364, 737446, +, 1083, 634011; cds-WP_032430148.1, NZ_KK737548.1, 737568, 740642, +, 3075, 5841286; cds-WP_023158026.1, NZ_KK737548.1, 740694, 741947, +, 1254, 1006959; cds-AF52_RS25890, NZ_KK737548.1, 742013, 742153, +,141,0; cds-AF52_RS25895, NZ_KK737548.1, 742168, 742863, -, 696, 2728; cds-AF52_RS25905, NZ_KK737548.1, 742956, 743322, +, 367, 15951; cds-AF52_RS07290, NZ_KK737548.1, 743401, 743690, +, 290, 37108; cds-WP_032430151.1, NZ_KK737548.1, 746080, 747084, +, 1005, 196375; cds-AF52_RS07280, NZ_KK737548.1, 747266, 747803, -, 538, 39105; cds-AF52_RS25915, NZ_KK737548.1, 748423, 748868, -, 446, 178635; cds-WP _004118832.1, NZ_KK737548.1, 749044, 750777, -, 1734, 131963; cds-WP _004118225.1, NZ_KK737548.1, 750785, 751732, -, 948, 7913; cds-WP _004152278.1, NZ_KK737548.1, 751777, 753381, -, 1605, 16545; cds-WP_004118227.1, NZ_KK737548.1, 753394, 754314, -, 921, 2588; cds-WP_004118228.1, NZ_KK737548.1, 754314, 755162, -, 849, 13601; cds-WP_004118229.1, NZ_KK737548.1, 755159, 755752, -, 594, 16685; cds-WP _004118840.1, NZ_KK737548.1, 755749, 756876, -, 1128, 40920; cds-WP_004118231.1, NZ_KK737548.1, 757161, 757328, -, 168, 9704; cds-WP_162551856.1, NZ_KK737548.1, 757257, 757469, +, 213, 10598; cds-AF52_RS07235, NZ_KK737548.1, 757470, 757955, +, 486, 54801; cds-WP _004197062.1, NZ_KK737548.1, 760634, 761155, +, 522, 125338; cds-WP_004197067.1, NZ_KK737548.1, 761152, 762105, +, 954, 67681; cds-WP_032430155.1, NZ_KK737548.1, 762198, 764522, +, 2325, 14189294; cds-WP _023157913.1, NZ_KK737548.1, 764567, 765469, +, 903, 3231750; cds-WP_032430156.1, NZ_KK737548.1, 765466, 766464, +, 999, 1939377; cds-WP _004118246.1, NZ_KK737548.1, 766461, 767417, +, 957, 671120; cds-WP_004152282.1, NZ_KK737548.1, 767418, 768185, +, 768, 373062; cds-WP_004118251.1, NZ_KK737548.1, 768284, 768577, +, 294, 159364; cds-AF52_RS25920, NZ_KK737548.1, 768652, 768879, +, 228, 138561; cds-WP_020806188.1-5, NZ_KK737548.1, 769118, 770098, -, 981, 246880; cds-WP _032430158.1, NZ_KK737548.1, 770398, 771066, +, 669, 5672; cds-WP _002885079.1, NZ_KK737548.1, 771455, 772804, +, 1350, 141057; cds-WP _002885082.1, NZ_KK737548.1, 772952, 774598, +, 1647, 203513; cds-WP_002885085.1, NZ_KK737548.1, 774761, 775642, -, 882, 91987; cds-WP _002885086.1, NZ_KK737548.1, 775748, 776155, +, 408, 16550; cds-WP_002885088.1, NZ_KK737548.1, 776152, 776841, +, 690, 37745; cds-AF52_RS0124245, NZ_KK737548.1, 777352, 777909, +, 558, 4; cds-WP _004210006.1, NZ_KK737548.1, 777970, 778659, +, 690, 6118; cds-WP_032428472.1, NZ_KK737548.1, 778613, 781192, +, 2580, 62010; cds-WP _004151734.1, NZ_KK737548.1, 781228, 781776, +, 549, 1529; cds-WP_002885139.1, NZ_KK737548.1, 781796, 782893, +, 1098, 15864; cds-WP _024623073.1, NZ_KK737548.1, 782941, 784593, -, 1653, 185052; cds-WP _004177812.1, NZ_KK737548.1, 784593, 784904, -, 312, 114208; cds-WP_002885145.1, NZ_KK737548.1, 785088, 787046, -, 1959, 4557317; cds-WP_004146658.1, NZ_KK737548.1, 787147, 787347, +, 201, 23239; cds-WP _004146659.1, NZ_KK737548.1, 787459, 788772, +, 1314, 253488; cds-WP_004151733.1, NZ_KK737548.1, 788809, 789495, -, 687, 127393; cds-WP_012737173.1, NZ_KK737548.1, 789725, 791872, -, 2148, 65155; cds-WP_023159071.1, NZ_KK737548.1, 792145, 792873, +, 729, 24017; cds-WP _004177809.1, NZ_KK737548.1, 792959, 793717, +, 759, 50780; cds-WP _002885155.1, NZ_KK737548.1, 793723, 794640, +, 918, 183357; cds-AF52_RS0124265, NZ_KK737548.1, 794765, 795772, -, 1008, 16827; cds-WP_002885156.1, NZ_KK737548.1, 795847, 796788, -, 942, 845; cds-WP _002885172.1, NZ_KK737548.1, 796819, 797817, -, 999, 441; cds-WP _002885173.1, NZ_KK737548.1, 797814, 799334, -, 1521, 0; cds-WP _021441042.1, NZ_KK737548.1, 799514, 801799, +, 2286, 4599; cds-WP _032430167.1, NZ_KK737548.1, 801870, 802223, +, 354, 29816; cds-WP_004210023.1, NZ_KK737548.1, 802245, 803003, -, 759, 9298; cds-WP _004151727.1, NZ_KK737548.1, 803019, 803582, -, 564, 41; cds-WP _009484481.1, NZ_KK737548.1, 803582, 804718, -, 1137, 2648; cds-WP_002885196.1, NZ_KK737548.1, 804715, 805395, -, 681, 123825; cds-WP_004177787.1, NZ_KK737548.1, 805503, 806261, -, 759, 13; cds-WP_002885202.1, NZ_KK737548.1, 806258, 807106, -, 849, 0; cds-WP _021441044.1, NZ_KK737548.1, 807099, 808163, -, 1065, 90; cds-WP _002885207.1, NZ_KK737548.1, 808164, 808748, -, 585, 94; cds-WP_020802927.1, NZ_KK737548.1, 808745, 809197, -, 453, 0; cds-WP_032428478.1, NZ_KK737548.1, 809198, 809923, -, 726, 42054; cds-WP _004177784.1, NZ_KK737548.1, 810079, 810513, -, 435, 81260; cds-WP_004177783.1, NZ_KK737548.1, 810742, 811077, -, 336, 247081; cds-WP _002885227.1, NZ_KK737548.1, 811622, 813124, +, 1503, 351556; cds-WP_004177781.1, NZ_KK737548.1, 813174, 814112, -, 939, 13833; cds-WP_032430168.1, NZ_KK737548.1, 814352, 815704, +, 1353, 3754; cds-WP _019724884.1, NZ_KK737548.1, 815798, 817213, +, 1416, 41889; cds-WP_004146677.1, NZ_KK737548.1, 817219, 817341, +, 123, 45198; cds-WP_004177778.1, NZ_KK737548.1, 817489, 818445, +, 957, 764271; cds-WP _004177777.1, NZ_KK737548.1, 818455, 818826, +, 372, 59010; cds-WP _002885324.1, NZ_KK737548.1, 819518, 819823, -, 306, 2619; cds-WP_021441050.1, NZ_KK737548.1, 819823, 820740, -, 918, 5633; cds-WP _020316611.1, NZ_KK737548.1, 820816, 820929, +, 114, 131; cds-WP _004221988.1, NZ_KK737548.1, 820886, 821569, -, 684, 15555; cds-WP _002885391.1, NZ_KK737548.1, 821795, 822580, +, 786, 58586; cds-WP _004177775.1, NZ_KK737548.1, 822626, 823075, -, 450, 66; cds-WP_002885393.1, NZ_KK737548.1, 823075, 824403, -, 1329, 39709; cds-WP _032430193.1, NZ_KK737548.1, 824396, 826411, -, 2016, 6886; cds-WP _072271759.1, NZ_KK737548.1, 826420, 828867, -, 2448, 29448; cds-AF52_RS06930, NZ_KK737548.1, 829500, 829859, +, 360, 67293; cds-WP _032430171.1, NZ_KK737548.1, 829892, 830562, +, 671, 169625; cds-AF52_RS27350, NZ_KK737548.1, 830663, 830977, +, 315, 122729; cds-AF52_RS0124285, NZ_KK737548.1, 831034, 831249, +, 216, 2932; cds-WP_004146686.1, NZ_KK737548.1, 831244, 831366, -, 123, 7616; cds-WP_004153693.1, NZ_KK737548.1, 831561, 831851, +, 291, 19; cds-WP _021441054.1, NZ_KK737548.1, 832920, 835007, -, 2088, 36944; cds-WP_002885410.1, NZ_KK737548.1, 835075, 835665, -, 591, 221619; cds-WP _032430173.1, NZ_KK737548.1, 835686, 837482, -, 1797, 434177; cds-WP_002885422.1, NZ_KK737548.1, 837458, 837781, -, 324, 22461; cds-WP _002885423.1, NZ_KK737548.1, 837906, 839207, -, 1302, 214260; cds-WP_002885424.1, NZ_KK737548.1, 839323, 840759, -, 1437, 5601066; cds-WP _002885425.1, NZ_KK737548.1, 841097, 841573, +, 477, 39334; cds-WP _009485424.1, NZ_KK737548.1, 841625, 842875, -, 1251, 25636; cds-WP _004152420.1, NZ_KK737548.1, 843108, 843401, +, 294, 3385628; cds-WP_002885441.1, NZ_KK737548.1, 843439, 845085, +, 1647, 22417959; cds-AF52_RS27360, NZ_KK737548.1, 846097, 846318, -, 222, 36; cds-WP_013815099.1-57, NZ_KK737548.1, 846353, 847321, +, 969, 74545; cds-AF52_RS25950, NZ_KK737548.1, 847378, 848127, -, 750, 4403; cds-WP _004186605.1, NZ_KK737548.1, 848177, 848857, -, 681, 298; cds-WP_004186607.1, NZ_KK737548.1, 848908, 849450, -, 543, 5; cds-WP_004222578.1, NZ_KK737549.1, 190, 258, +, 69, 23694; cds-WP _002887848.1, NZ_KK737549.1, 340, 2802, +, 2463, 412549; cds-WP_002887853.1, NZ KK737549.1, 2804, 3733, +, 930, 114769; cds-WP _002887863.1, NZ KK737549.1, 3737, 5017, +, 1281, 125557; cds-WP_004222583.1, NZ_KK737549.1, 5332, 5709, +, 378, 21959; cds-WP_004178561.1, NZ_KK737549.1, 5778, 6551, -, 774, 252818; cds-WP_002887871.1, NZ_KK737549.1, 6629, 8059, -, 1431, 34858; cds-WP_002887897.1, NZ KK737549.1, 8262, 9215, +, 954, 4733179; cds-WP _002887898.1, NZ_KK737549.1, 9315, 9902, +, 588, 161965; cds-WP_021441173.1, NZ_KK737549.1, 10022, 11326, +, 1305, 84681; cds-WP_004177461.1, NZ_KK737549.1, 11389, 11955, -, 567, 6890; cds-WP _021441175.1, NZ KK737549.1, 12044, 12793, -, 750, 13107; cds-WP_002887909.1, NZ_KK737549.1, 12821, 13225, -, 405, 122; cds-WP_004146997.1, NZ_KK737549.1, 13611, 15527, +, 1917, 10358104; cds-WP _002887955.1, NZ_KK737549.1, 15615, 16748, +, 1134, 1015878; cds-WP _004192071.1, NZ KK737549.1, 16926, 18101, +, 1176, 650335; cds-WP _002887961.1, NZ_KK737549.1, 18156, 19052, +, 897, 590231; cds-WP_002887965.1, NZ_KK737549.1, 19172, 19435, -, 264, 708935; cds-WP _002887969.1, NZ_KK737549.1, 19765, 20703, +, 939, 765531; cds-WP _032430302.1, NZ KK737549.1, 20747, 23563, +, 2817, 4756655; cds-WP_002887973.1, NZ KK737549.1, 23563, 24063, +, 501, 185115; cds-WP _002887976.1, NZ_KK737549.1, 24179, 24628, +, 450, 321586; cds-WP _002887979.1, NZ_KK737549.1, 24630, 25580, +, 951, 767522; cds-WP _032430200.1, NZ_KK737549.1, 25647, 26561, +, 915, 328683; cds-WP _032428524.1, NZ KK737549.1, 26592, 26957, +, 366, 93247; cds-WP_021441178.1, NZ_KK737549.1, 26905, 27834, -, 930, 97445; cds-WP_004192057.1, NZ_KK737549.1, 27927, 28556, +, 630, 445489; cds-WP_004222618.1, NZ_KK737549.1, 28562, 29266, -, 705, 1010562; cds-WP_004151379.1, NZ_KK737549.1, 29259, 30887, -, 1629, 371534; cds-WP _032430201.1, NZ KK737549.1, 31059, 32360, -, 1302, 7372; cds-WP_021441180.1, NZ KK737549.1, 32376, 34154, -, 1779, 10; cds-WP _012540458.1, NZ_KK737549.1, 34170, 34421, -, 252, 0; cds-WP_004151375.1, NZ KK737549.1, 34577, 35917, -, 1341, 7081; cds-WP _004222626.1, NZ KK737549.1, 36174, 37202, +, 1029, 122; cds-WP _004151373.1, NZ_KK737549.1, 37233, 37526, +, 294, 0; cds-WP _004151372.1, NZ_KK737549.1, 37523, 38392, +, 870, 0; cds-WP _032428528.1, NZ_KK737549.1, 38403, 39929, +, 1527, 1014; cds-WP_004222628.1, NZ_KK737549.1, 39948, 40856, +, 909, 1768; cds-WP_021441183.1, NZ_KK737549.1, 41085, 41906, +, 822, 271359; cds-WP_004212571.1, NZ_KK737549.1, 42364, 43512, +, 1149, 2133036; cds-WP _002888056.1, NZ KK737549.1, 43530, 46754, +, 3225, 7750280; cds-WP_002888059.1, NZ_KK737549.1, 46924, 47157, +, 234, 23476; cds-WP_002888068.1, NZ_KK737549.1, 47289, 47822, +, 534, 151935; cds-WP_004145992.1, NZ KK737549.1, 47815, 49680, +, 1866, 120987; cds-WP _002888320.1, NZ KK737549.1, 49854, 50333, +, 480, 163580; cds-WP _002888321.1, NZ KK737549.1, 50586, 51434, -, 849, 202640; cds-WP _002888323.1, NZ KK737549.1, 51439, 51816, -, 378, 209920; cds-WP_002888324.1, NZ KK737549.1, 51819, 52640, -, 822, 429410; cds-WP _032430205.1, NZ_KK737549.1, 52637, 53626, -, 990, 1015031; cds-WP _002888329.1, NZ KK737549.1, 53632, 54918, -, 1287, 1725030; cds-WP_032103829.1, NZ_KK737549.1, 54973, 57321, -, 2349, 2067987; cds-WP _002888332.1, NZ_KK737549.1, 57565, 58392, +, 828, 274658; cds-WP_002888337.1, NZ_KK737549.1, 58425, 59123, -, 699, 17731; cds-WP_004192048.1, NZ_KK737549.1, 59113, 60738, -, 1626, 135652; cds-WP_002888345.1, NZ KK737549.1, 61063, 62427, +, 1365, 6757; cds-WP_021441189.1, NZ KK737549.1, 62607, 63146, +, 540, 160515; cds-WP_002888348.1, NZ_KK737549.1, 63209, 63868, -, 660, 181842; cds-WP_032428530.1, NZ KK737549.1, 63881, 66787, -, 2907, 1306674; cds-WP_021441191.1, NZ KK737549.1, 66975, 69332, -, 2358, 371699; cds-WP _002888353.1, NZ KK737549.1, 69499, 70194, -, 696, 4; cds-WP_021441192.1, NZ_KK737549.1, 70332, 71834, -, 1503, 120892; cds-WP_023279864.1, NZ_KK737549.1, 71845, 73554, -, 1710, 13143; cds-WP _004145972.1, NZ KK737549.1, 73892, 74737, +, 846, 201388; cds-WP _002888373.1, NZ_KK737549.1, 74866, 75633, +, 768, 25011; cds-WP _032430207.1, NZ KK737549.1, 75620, 76321, -, 702, 12190; cds-WP _002888430.1, NZ_KK737549.1, 76305, 77915, -, 1611, 109717; cds-WP _032428532.1, NZ KK737549.1, 77891, 78874, -, 984, 536525; cds-AF52_RS0124360, NZ KK737549.1, 79164, 80819, -, 1656, 220236; cds-WP_002888448.1, NZ_KK737549.1, 80907, 81059, +, 153, 916; cds-WP _004147046.1, NZ KK737549.1, 81327, 82511, +, 1185, 25337; cds-WP_004147047.1, NZ_KK737549.1, 82545, 83855, -, 1311, 62235; cds-WP_013815099.1-58, NZ_KK737549.1, 84110, 85078, +, 969, 97463; cds-WP_002888455.1, NZ_KK737549.1, 85153, 86049, +, 897, 12239; cds-WP_004177420.1, NZ_KK737549.1, 86087, 86842, -, 756, 38347; cds-WP _032428534.1, NZ KK737549.1, 86873, 88087, -, 1215, 2254; cds-WP _002888460.1, NZ_KK737549.1, 88098, 88961, -, 864, 163; cds-WP _004147052.1, NZ_KK737549.1, 88973, 91006, -, 2034, 5852; cds-WP _002888481.1, NZ_KK737549.1, 91402, 92007, -, 606, 151420; cds-WP _021441196.1, NZ KK737549.1, 92018, 93418, -, 1401, 188181; cds-WP _002888487.1, NZ KK737549.1, 93421, 94512, -, 1092, 162916; cds-WP_023287248.1, NZ KK737549.1, 94512, 96083, -, 1572, 67274; cds-WP _004222662.1, NZ_KK737549.1, 96179, 96265, -,
87, 197; cds-WP_148673689.1, NZ_KK737549.1, 96879, 97847, +, 969, 7756; cds-WP_002888534.1, NZ_KK737549.1, 98242, 99966, +, 1725, 106634; cds-WP_002888536.1, NZ_KK737549.1, 99969, 100460, +, 492, 34465; cds-WP_004145952.1, NZ_KK737549.1, 100641, 101645, +, 1005,844064; cds-WP_002888541.1, NZ_KK737549.1, 102286, 102744, +, 459, 502965; cds-WP_002888547.1, NZ_KK737549.1, 102747, 103688, +, 942, 1003916; cds-WP_002888550.1, NZ_KK737549.1, 103685, 104050, +, 366, 378996; cds-WP_032430210.1, NZ_KK737549.1, 104066, 105832, +, 1767, 1169896; cds-WP_004182923.1, NZ_KK737549.1, 105819, 107306, +, 1488, 928773; cds-WP_004177411.1, NZ_KK737549.1, 107303, 108661, +, 1359, 506708; cds-WP_002888562.1, NZ_KK737549.1, 108655, 109737, +, 1083, 460631; cds-WP_004151364.1, NZ_KK737549.1, 109740, 111056, +, 1317, 484869; cds-WP_002888565.1, NZ_KK737549.1, 111056, 112330, +, 1275, 702879; cds-WP_002888566.1, NZ_KK737549.1, 112327, 113397, +, 1071, 233394; cds-WP_004145947.1, NZ_KK737549.1, 113445, 114920, +, 1476, 660188; cds-WP_002888623.1, NZ_KK737549.1, 114913, 115833, +, 921, 398617; cds-WP_002888624.1, NZ_KK737549.1, 115835, 116665, +, 831, 691946; cds-WP_002888625.1, NZ_KK737549.1, 116662, 117924, +, 1263, 1053512; cds-WP_002888628.1, NZ_KK737549.1, 117987, 119138, +, 1152, 2662650; cds-WP_032430211.1, NZ_KK737549.1, 119239, 120156, +, 918, 4078659; cds-WP_004152922.1, NZ_KK737549.1, 120473, 120976, +, 504, 255914; cds-WP_002888638.1, NZ_KK737549.1, 121039, 123744, +, 2706, 2716537; cds-WP_004145942.1, NZ_KK737549.1, 123803, 124195, +, 393, 65160; cds-WP_002888644.1, NZ_KK737549.1, 124248, 124442, -, 195, 70345; cds-WP_004178596.1, NZ_KK737549.1, 124452, 125195, -, 744, 180488; cds-WP_002888649.1, NZ_KK737549.1, 125195, 125815, -, 621, 89111; cds-WP_004145938.1, NZ_KK737549.1, 126159, 127202, +, 1044, 593109; cds-WP_021441203.1, NZ_KK737549.1, 127239, 128444, -, 1206, 11093; cds-WP_004145935.1, NZ_KK737549.1, 128434, 129819, -, 1386, 15587; cds-WP_002888683.1, NZ_KK737549.1, 129830, 130261, -, 432, 61983; cds-WP_002888684.1, NZ_KK737549.1, 130443, 131336, -, 894, 79927; cds-WP_002888692.1, NZ_KK737549.1, 131470, 132033, +, 564, 96632; cds-WP_002888694.1, NZ_KK737549.1, 132030, 132884, +, 855, 131469; cds-WP_021441204.1, NZ_KK737549.1, 133120, 134070, -, 951, 109998; cds-WP_032428537.1, NZ_KK737549.1, 134070, 135476, -, 1407, 151204; cds-WP_004147090.1, NZ_KK737549.1, 135661, 137031, -, 1371, 79821; cds-WP_002888699.1, NZ_KK737549.1, 137675, 138454, +, 780, 180064; cds-WP_032430213.1, NZ_KK737549.1, 138591, 141254, +, 2664, 6767104; cds-WP_021441205.1, NZ_KK737549.1, 141269, 143167, +, 1899, 2284319; cds-WP_002888731.1, NZ_KK737549.1, 143374, 144798, +, 1425, 14502708; cds-WP_004177403.1, NZ_KK737549.1, 144963, 145247, +, 285, 2574; cds-WP_002888733.1, NZ_KK737549.1, 145293, 146099, -, 807, 997; cds-WP_032430214.1, NZ_KK737549.1, 146111, 147721, -, 1611, 33580; cds-WP_002888735.1, NZ_KK737549.1, 148153, 150750, +, 2598, 27379809; cds-WP_002888737.1, NZ_KK737549.1, 150937, 151299, +, 363, 63052; cds-WP_002888739.1, NZ_KK737549.1, 151372, 152166, -, 795, 407131; cds-WP_002888741.1, NZ_KK737549.1, 152191, 153051, -, 861, 385681; cds-WP_072072754.1, NZ_KK737549.1, 153201, 154394, -, 1194, 25097; cds-AF52_RS28060, NZ_KK737549.1, 154404, 154643, -, 240, 9265; cds-WP_002888804.1, NZ_KK737549.1, 155225, 155635, -, 411, 9411; cds-WP_002888808.1, NZ_KK737549.1, 155882, 156229, -, 348, 63767; cds-WP_032430215.1, NZ_KK737549.1, 156375, 157973, +, 1599, 39129; cds-WP_002888816.1, NZ_KK737549.1, 158057, 160447, -, 2391, 549148; cds-WP_012542835.1, NZ_KK737549.1, 160528, 160647, +, 120, 2199; cds-WP_002888819.1, NZ_KK737549.1, 160652, 161188, +, 537, 154074; cds-WP_002888821.1, NZ_KK737549.1, 161248, 161910, -, 663, 280673; cds-WP_002888823.1, NZ_KK737549.1, 162095, 163021, +, 927, 251694; cds-WP_004204389.1, NZ_KK737549.1, 163018, 163788, +, 771, 153501; cds-WP_023282187.1, NZ_KK737549.1, 163911, 164351, +, 441, 40044; cds-WP_004145907.1, NZ_KK737549.1, 164410, 165663, +, 1254, 346272; cds-WP_002888834.1, NZ_KK737549.1, 165699, 166079, -, 381, 244172; cds-WP_002888835.1, NZ_KK737549.1, 166172, 167026, -, 855, 313840; cds-WP_002888841.1, NZ_KK737549.1, 167038, 167829, -, 792, 88013; cds-WP_004145905.1, NZ_KK737549.1, 167950, 168429, -, 480, 149581; cds-WP_071526609.1, NZ_KK737549.1, 168426, 169820, -, 1395, 563060; cds-WP_021441212.1, NZ_KK737549.1, 169883, 170764, -, 882, 796606; cds-WP_002888845.1, NZ_KK737549.1, 170824, 171279, -, 456, 1561582; cds-WP_032430216.1, NZ_KK737549.1, 171442, 172158, -, 717, 209417; cds-WP_004145901.1, NZ_KK737549.1, 172158, 172694, -, 537, 8043; cds-WP_023282188.1, NZ_KK737549.1, 172767, 175196, +, 2430, 68336; cds-WP_021441214.1, NZ_KK737549.1, 175244, 176044, -, 801, 3045; cds-WP_021441215.1, NZ_KK737549.1, 176041, 176787, -, 747, 189; cds-WP_004151943.1, NZ_KK737549.1, 176777, 177262, -, 486, 0; cds-WP_021441216.1, NZ_KK737549.1, 177255, 178448, -, 1194, 0; cds-WP_004183041.1, NZ_KK737549.1, 178435, 179421, -, 987, 72; cds-WP_032428541.1, NZ_KK737549.1, 179418, 180014, -, 597, 20; cds-WP_002889025.1, NZ_KK737549.1, 180011, 180376, -, 366, 0; cds-WP_021441218.1, NZ_KK737549.1, 180379, 180888, -, 510, 29; cds-WP_002889054.1, NZ_KK737549.1, 180888, 181310, -, 423, 16136; cds-WP_004177382.1, NZ_KK737549.1, 181331, 182545, -, 1215,3214; cds-WP_004145888.1, NZ_KK737549.1, 182547, 184040, -, 1494, 1; cds-WP_032430217.1, NZ_KK737549.1, 184037, 186010, -, 1974, 7652; cds-WP_004177377.1, NZ_KK737549.1, 186020, 186862, -, 843, 2050; cds-WP_004219434.1, NZ_KK737549.1, 186847, 186984, -, 138, 380; cds-WP_023286046.1, NZ_KK737549.1, 187169, 190477, +, 3309, 166060; cds-WP_021441221.1, NZ_KK737549.1, 190734, 191291, +, 558, 37241; cds-WP_004194926.1, NZ_KK737549.1, 191330, 191704, -, 375, 52731; cds-WP_004147125.1, NZ_KK737549.1, 191767, 191901, -, 135, 1100; cds-WP_002889208.1, NZ_KK737549.1, 192292, 194844, +, 2553, 1187198; cds-WP_004178624.1, NZ_KK737549.1, 195079, 197286, +, 2208, 461560; cds-WP_002889212.1, NZ_KK737549.1, 197332, 198129, +, 798, 64349; cds-WP_004178627.1, NZ_KK737549.1, 198129, 199019, +, 891, 64099; cds-WP_032430218.1, NZ_KK737549.1, 199016, 200998, +, 1983, 64559; cds-WP_002889272.1, NZ_KK737549.1, 201135, 202415, -, 1281, 175957; cds-WP_004151932.1, NZ_KK737549.1, 202596, 204014, +, 1419, 98124; cds-WP_002889275.1, NZ_KK737549.1, 204096, 204440, +, 345, 113317; cds-WP_002889277.1, NZ_KK737549.1, 204512, 205135, -, 624, 20798; cds-WP_002889279.1, NZ_KK737549.1, 205172, 205972, -, 801, 221441; cds-WP_002889282.1, NZ_KK737549.1, 205965, 206663, -, 699, 322932; cds-WP_004145871.1, NZ_KK737549.1, 206747, 208261, +, 1515, 385447; cds-WP_004177363.1, NZ_KK737549.1, 208396, 209829, +, 1434, 2121361; cds-WP_002889289.1, NZ_KK737549.1, 209996, 211153, +, 1158, 316129; cds-WP_002889292.1, NZ_KK737549.1, 211272, 211658, -, 387, 256641; cds-WP_002889295.1, NZ_KK737549.1, 211766, 212590, -, 825, 520771; cds-WP_004151931.1, NZ_KK737549.1, 212621, 215284, -, 2664, 561080; cds-WP_004177362.1, NZ_KK737549.1, 215342, 216136, -, 795, 1111066; cds-WP_002889299.1, NZ_KK737549.1, 216459, 217184, +, 726, 18849230; cds-WP_004151930.1, NZ_KK737549.1, 217304, 218155, +, 852, 5254594; cds-WP_002889306.1, NZ_KK737549.1, 218305, 219030, +, 726, 2075746; cds-WP_002889308.1, NZ_KK737549.1, 219181, 219738, +, 558, 1751119; cds-WP_004178642.1, NZ_KK737549.1, 219967, 221169, +, 1203, 313381; cds-WP_002889316.1, NZ_KK737549.1, 221407, 222165, +, 759, 1021112; cds-WP_032430220.1, NZ_KK737549.1, 222178, 223035, +, 858, 590514; cds-WP_004177358.1, NZ_KK737549.1, 223047, 224399, +, 1353, 3106135; cds-WP_004145860.1, NZ_KK737549.1, 224431, 226860, +, 2430, 8487188; cds-WP_004178645.1, NZ_KK737549.1, 226982, 227467, +, 486, 1608172; cds-WP_002889327.1, NZ_KK737549.1, 227471, 228496, +, 1026, 2260246; cds-WP_004145858.1, NZ_KK737549.1, 228602, 229057, +, 456, 640910; cds-WP_004178647.1, NZ_KK737549.1, 229061, 229849, +, 789, 1344052; cds-WP_002889374.1, NZ_KK737549.1, 229849, 231000, +, 1152, 249780; cds-WP_021441229.1, NZ_KK737549.1, 230997, 231596, +, 600, 103599; cds-WP_004177353.1, NZ_KK737549.1, 231614, 235096, +, 3483, 655506; cds-WP_004145855.1, NZ_KK737549.1, 235109, 236068, +, 960, 710937; cds-WP_021441230.1, NZ_KK737549.1, 236182, 238335, +, 2154, 285486; cds-WP_021441231.1, NZ_KK737549.1, 238382, 238771, +, 390, 262302; cds-WP_009309280.1, NZ_KK737549.1, 238825, 240138, +, 1314, 123806; cds-WP_002889424.1, NZ_KK737549.1, 240152, 240412, -, 261, 98862; cds-WP_002889429.1, NZ_KK737549.1, 240399, 240599, -, 201, 82280; cds-WP_009309281.1, NZ_KK737549.1, 240796, 241341, +, 546, 30829; cds-WP_002889433.1, NZ_KK737549.1, 241338, 241751, +, 414, 27647; cds-WP_002889435.1, NZ_KK737549.1, 241794, 242492, +, 699, 166067; cds-WP_004212714.1, NZ_KK737549.1, 242624, 243466, -, 843, 194511; cds-WP_004151928.1, NZ_KK737549.1, 243525, 245243, -, 1719, 1924168; cds-WP_032430223.1, NZ_KK737549.1, 245356, 246063, -, 708, 268838; cds-WP_002889443.1, NZ_KK737549.1, 246060, 246467, -, 408, 153146; cds-WP_002889445.1, NZ_KK737549.1, 246575, 247390, -, 816, 332397; cds-WP_002889448.1, NZ_KK737549.1, 247434, 248087, -, 654, 88125; cds-WP_002889450.1, NZ_KK737549.1, 248080, 249111, -, 1032, 174588; cds-WP_002889452.1, NZ_KK737549.1, 249300, 249866, +, 567, 125613; cds-WP_012737430.1, NZ_KK737549.1, 256451, 257254, +, 804, 188396; cds-WP_002889616.1, NZ_KK737549.1, 257300, 258205, -, 906, 1799; cds-WP_021441232.1, NZ_KK737549.1, 258310, 259494, +, 1185, 4959; cds-WP_002889629.1, NZ_KK737549.1, 259597, 260394, +, 798, 152691; cds-WP_021441233.1, NZ_KK737549.1, 260486, 261256, +, 771, 7298; cds-WP_002889632.1, NZ_KK737549.1, 261314, 262681, -, 1368, 421394; cds-WP_032428550.1, NZ_KK737549.1, 262753, 263508, -, 756, 84437; cds-WP_002889685.1, NZ_KK737549.1, 263541, 264263, +, 723, 162501; cds-WP_002889686.1, NZ_KK737549.1, 264260, 264727, -, 468, 31252; cds-WP_023286054.1, NZ_KK737549.1, 264783, 265523, +, 741, 32421; cds-WP_032430225.1, NZ_KK737549.1, 265793, 266674, -, 882, 61735; cds-WP_004178672.1, NZ_KK737549.1, 266778, 267368, +, 591, 4469; cds-WP_002889689.1, NZ_KK737549.1, 267399, 268169, -, 771, 24206; cds-WP_021441237.1, NZ_KK737549.1, 268347, 270791, -, 2445, 1660237; cds-WP_004145825.1, NZ_KK737549.1, 271031, 271612, +, 582, 527877; cds-WP_002889696.1, NZ_KK737549.1, 271687, 272454, +, 768, 373734; cds-WP_002889699.1, NZ_KK737549.1, 272425, 273165, -, 741, 617590; cds-WP_002889702.1, NZ_KK737549.1, 273577, 274920, +, 1344, 491045; cds-WP_002889703.1, NZ_KK737549.1, 274924, 276162, +, 1239, 398615; cds-WP_004177336.1, NZ_KK737549.1, 276155, 276949, +, 795, 217564; cds-WP_002889716.1, NZ_KK737549.1, 276942, 277580, +, 639, 545235; cds-WP_004177335.1, NZ_KK737549.1, 277587, 278183, +, 597, 244642; cds-WP_021441239.1, NZ_KK737549.1, 278196, 279419, +, 1224, 373936; cds-WP_004178678.1, NZ_KK737549.1, 279422, 279637, +, 216, 9928; cds-WP_021441240.1, NZ_KK737549.1, 280018, 280878, -, 861, 23909; cds-WP_002889726.1, NZ_KK737549.1, 281038, 282093, +, 1056, 185369; cds-WP_002889727.1, NZ_KK737549.1, 282096, 282548, +, 453, 3010; cds-WP_004152033.1, NZ_KK737549.1, 282591, 284048, -, 1458, 1250572; cds-WP_002889733.1, NZ_KK737549.1, 284306, 284764, +, 459, 247995; cds-WP_002889742.1, NZ_KK737549.1, 284870, 286114, +, 1245, 309863; cds-WP_004177332.1, NZ_KK737549.1, 286172, 286570, +, 399, 164036; cds-WP_004177331.1, NZ_KK737549.1, 286656, 287348, -, 693, 60306; cds-WP_032430227.1, NZ_KK737549.1, 287394, 288794, -, 1401, 73836; cds-WP_032430228.1, NZ_KK737549.1, 288944, 290227, -, 1284, 14890; cds-WP_032430229.1, NZ_KK737549.1, 290240, 291688, -, 1449, 69785; cds-WP_002889846.1, NZ_KK737549.1, 291713, 292981, -, 1269, 285818; cds-WP_002889847.1, NZ_KK737549.1, 293009, 293986, -, 978, 7165; cds-WP_032430230.1, NZ_KK737549.1, 294448, 294705, +, 258, 238647; cds-WP_032430232.1, NZ_KK737549.1, 294763, 295512, +, 750, 521946; cds-WP_032421047.1, NZ_KK737549.1, 295784, 296248, +, 465, 340478; cds-WP_009309773.1, NZ_KK737549.1, 296439, 297173, -, 735, 151510; cds-WP_021441244.1, NZ_KK737549.1, 297139, 298473, -, 1335, 38137; cds-WP_032430234.1, NZ_KK737549.1, 298478, 301033, -, 2556, 25580; cds-WP_009309770.1, NZ_KK737549.1, 301017, 301775, -, 759, 977; cds-AF52_RS05525, NZ_KK737549.1, 301835, 302236, -,
402, 41; cds-AF52_RS27385, NZ_KK737549.1, 302271, 302442, +, 172, 7547; cds-AF52_RS27390, NZ_KK737549.1, 303421, 303558, +, 138, 37873; cds-WP_050484291.1, NZ_KK737549.1, 303624, 304661, +, 1038, 159782; cds-WP_004222859.1, NZ_KK737549.1, 304706, 305272, -, 567, 183199; cds-WP_023279253.1, NZ_KK737549.1, 305685, 306554, +, 870, 180; cds-WP_021441254.1, NZ_KK737549.1, 306650, 307315, +, 666, 2842; cds-WP_124053765.1, NZ_KK737549.1, 307782, 307952, -, 171, 12; cds-WP_016531458.1, NZ_KK737549.1, 308463, 309347, -, 885, 1013; cds-WP_002890001.1, NZ_KK737549.1, 309344, 310216, -, 873, 0; cds-WP_002890002.1, NZ_KK737549.1, 310283, 311221, -, 939, 793; cds-WP_004177295.1, NZ_KK737549.1, 311244, 312086, -, 843, 86; cds-WP_013815099.1-59, NZ_KK737549.1, 312505, 313473, -, 969, 84338; cds-WP_013815099.1-60, NZ_KK737549.1, 313954, 314922, +, 969, 91958; cds-AF52_RS27405, NZ_KK737549.1, 314979, 315062, -, 84, 582; cds-WP_004144574.1, NZ_KK737549.1, 315352, 316455, +, 1104, 249622; cds-WP_004147253.1, NZ_KK737549.1, 316466, 317719, +, 1254, 460212; cds-AF52_RS27410, NZ_KK737549.1, 318130, 318266, -, 137, 3789; cds-AF52_RS27415, NZ_KK737549.1, 319963, 320115, -, 153, 5721; cds-AF52_RS05440, NZ_KK737549.1, 320150, 320659, +, 510, 32; cds-WP_032430236.1, NZ_KK737549.1, 320718, 321386, +, 669, 13350; cds-WP_032430238.1, NZ_KK737549.1, 321412, 323937, +, 2526, 0; cds-WP_002890060.1, NZ_KK737549.1, 323927, 325570, +, 1644, 1230; cds-WP_002890061.1, NZ_KK737549.1, 325539, 326249, +, 711, 0; cds-WP_004178714.1, NZ_KK737549.1, 326253, 326942, -, 690, 0; cds-WP_032430239.1, NZ_KK737549.1, 326932, 327684, -, 753, 26; cds-WP_004151355.1, NZ_KK737549.1, 327681, 328499, -, 819, 0; cds-WP_002890106.1, NZ_KK737549.1, 328501, 329310, -, 810, 29; cds-WP_013815099.1-61, NZ_KK737549.1, 329684, 330652, +, 969, 65957; cds-WP_002890108.1, NZ_KK737549.1, 330723, 330893, +, 171, 195; cds-WP_002890126.1, NZ_KK737549.1, 331010, 331705, -, 696, 14454; cds-WP_004151354.1, NZ_KK737549.1, 331698, 332480, -, 783, 814; cds-WP_004144594.1, NZ_KK737549.1, 332467, 333516, -, 1050, 120; cds-WP_025861891.1, NZ_KK737549.1, 333516, 334415, -, 900, 503; cds-WP_004147267.1, NZ_KK737549.1, 334435, 335541, -, 1107, 99; cds-WP_009485305.1, NZ_KK737549.1, 335534, 335677, +, 144, 0; cds-WP_004147268.1, NZ_KK737549.1, 336117, 336893, -, 777, 224; cds-WP_032430241.1, NZ_KK737549.1, 336890, 338278, -, 1389, 3658; cds-WP_032430243.1, NZ_KK737549.1, 338288, 339667, -, 1380, 671; cds-WP_021441263.1, NZ_KK737549.1, 339730, 339858, +, 129, 0; cds-WP_002890161.1, NZ_KK737549.1, 339941, 341350, +, 1410, 86264; cds-WP_032430245.1, NZ_KK737549.1, 341337, 342269, +, 933, 3695; cds-WP_032430246.1, NZ_KK737549.1, 342509, 343471, +, 963, 0; cds-WP_004183141.1, NZ_KK737549.1, 343483, 344250, +, 768, 38; cds-WP_004144603.1, NZ_KK737549.1, 344247, 345074, +, 828, 321; cds-WP_004147277.1, NZ_KK737549.1, 345071, 345922, +, 852, 4618; cds-WP_032430247.1, NZ_KK737549.1, 346096, 347814, -, 1719, 18; cds-WP_002890201.1, NZ_KK737549.1, 348267, 349241, -, 975, 415278; cds-WP_004151350.1, NZ_KK737549.1, 349348, 350508, -, 1161, 326728; cds-WP_004151349.1, NZ_KK737549.1, 350727, 351239, +, 513, 157439; cds-WP_002890204.1, NZ_KK737549.1, 351357, 352577, +, 1221, 249861; cds-WP_004183146.1, NZ_KK737549.1, 352596, 353702, +, 1107, 130413; cds-WP_002890210.1, NZ_KK737549.1, 353699, 354001, -, 303, 53454; cds-WP_002890215.1, NZ_KK737549.1, 354254, 354457, +, 204, 33679; cds-WP_004151348.1, NZ_KK737549.1, 354490, 355587, -, 1098, 188050; cds-WP_004212277.1, NZ_KK737549.1, 355687, 356370, +, 684, 20267; cds-WP_002890219.1, NZ_KK737549.1, 356540, 357739, +, 1200, 0; cds-WP_002890221.1, NZ_KK737549.1, 358039, 358299, +, 261, 53493; cds-WP_004232385.1, NZ_KK737549.1, 358389, 359819, +, 1431, 217240; cds-WP_002890225.1, NZ_KK737549.1, 359911, 360228, +, 318, 6450; cds-WP_004177276.1, NZ_KK737549.1, 360296, 361105, -, 810, 228727; cds-WP_002890230.1, NZ_KK737549.1, 361222, 361680, +, 459, 9212; cds-WP_032430252.1, NZ_KK737549.1, 361677, 362387, -, 711, 601240; cds-WP_002890276.1, NZ_KK737549.1, 362716, 363249, +, 534, 71070; cds-WP_002890278.1, NZ_KK737549.1, 363321, 363512, +, 192, 204882; cds-WP_004144619.1, NZ_KK737549.1, 363763, 364443, +, 681, 29222; cds-WP_002890284.1, NZ_KK737549.1, 364521, 364805, +, 285, 157879; cds-WP_002890285.1, NZ_KK737549.1, 364877, 365788, -, 912, 210016; cds-WP_032430253.1, NZ_KK737549.1, 365880, 366794, +, 915, 83998; cds-WP_032430254.1, NZ_KK737549.1, 366868, 370005, -, 3138, 159653; cds-WP_032430256.1, NZ_KK737549.1, 370002, 371207, -, 1206, 66082; cds-WP_002890343.1, NZ_KK737549.1, 371390, 372079, +, 690, 287455; cds-WP_002890344.1, NZ_KK737549.1, 372101, 373396, +, 1296, 283053; cds-WP_032430258.1, NZ_KK737549.1, 373810, 375129, +, 1320, 142392; cds-WP_002890348.1, NZ_KK737549.1, 375216, 376589, +, 1374, 111308; cds-WP_032430260.1, NZ_KK737549.1, 376741, 378558, +, 1818, 24568; cds-WP_002890353.1, NZ_KK737549.1, 378555, 379457, -, 903, 1946; cds-WP_002890357.1, NZ_KK737549.1, 379724, 380365, +, 642, 2; cds-WP_002890365.1, NZ_KK737549.1, 380552, 381097, +, 546, 0; cds-WP_023286084.1, NZ_KK737549.1, 381122, 382990, +, 1869, 1257; cds-WP_004218830.1, NZ_KK737549.1, 383049, 383903, -, 855, 14; cds-WP_032430262.1, NZ_KK737549.1, 383939, 384883, -, 945, 985; cds-WP_013815099.1-62, NZ_KK737549.1, 384903, 385871, +, 969, 75759; cds-AF52_RS05135, NZ_KK737549.1, 386033, 387202, -, 1170, 22580; cds-WP_013815099.1-63, NZ_KK737549.1, 387236, 388204, +, 969, 77881; cds-AF52_RS28095, NZ_KK737549.1, 388263, 388406, +, 144, 235; cds-WP_019704181.1, NZ_KK737549.1, 388474, 388707, +, 234, 0; cds-WP_032430264.1, NZ_KK737549.1, 388707, 388934, +, 228, 34; cds-AF52_RS05115, NZ_KK737549.1, 388931, 389551, +, 621, 4024; cds-WP_013815099.1-64, NZ_KK737549.1, 389609, 390577, -, 969, 79007; cds-WP_021441029.1, NZ_KK737549.1, 390933, 392513, +, 1581, 3563; cds-WP_004151744.1, NZ_KK737549.1, 392638, 393162, -, 525, 350301; cds-WP_004146620.1, NZ_KK737549.1, 393414, 396239, +, 2826, 4592608; cds-WP_002884953.1, NZ_KK737549.1, 396240, 396596, -, 357, 58371; cds-WP_002884951.1, NZ_KK737549.1, 396600, 397016, -, 417, 20168; cds-WP_004151745.1, NZ_KK737549.1, 397147, 397860, -, 714, 69562; cds-WP_009485000.1, NZ_KK737549.1, 398051, 399244, -, 1194, 161312; cds-WP_004214117.1, NZ_KK737549.1, 399421, 400500, -, 1080, 62826; cds-WP_002884942.1, NZ_KK737549.1, 400532, 401947, -, 1416, 260885; cds-WP_032428470.1, NZ_KK737549.1, 402117, 403100, +, 984, 131099; cds-WP_009484997.1, NZ_KK737549.1, 403282, 403524, -, 243, 724; cds-WP_004214118.1, NZ_KK737549.1, 403664, 404662, -, 999, 8686; cds-WP_002884935.1, NZ_KK737549.1, 404763, 405251, -, 489, 21365; cds-WP_002884931.1, NZ_KK737549.1, 405501, 406016, +, 516, 101789; cds-WP_004151747.1, NZ_KK737549.1, 406112, 406321, -, 210, 158785; cds-WP_004219133.1, NZ_KK737549.1, 406387, 406536, +, 150, 36669; cds-WP_004151748.1, NZ_KK737549.1, 406556, 407872, -, 1317, 430631; cds-WP_002884822.1, NZ_KK737549.1, 407942, 408550, -, 609, 1550542; cds-WP_002884819.1, NZ_KK737549.1, 408673, 409041, -, 369, 56028; cds-WP_004178269.1, NZ_KK737549.1, 409170, 411593, +, 2424, 1290721; cds-WP_002884808.1, NZ_KK737549.1, 411642, 412508, -, 867, 151315; cds-WP_002884807.1, NZ_KK737549.1, 412521, 413018, -, 498, 295385; cds-WP_032428466.1, NZ_KK737549.1, 413206, 414129, -, 924, 1918850; cds-WP_002884747.1, NZ_KK737549.1, 414244, 415533, -, 1290, 10417857; cds-WP_002884743.1, NZ_KK737549.1, 415617, 416726, -, 1110, 2808619; cds-WP_002884740.1, NZ_KK737549.1, 417097, 418287, +, 1191, 6495824; cds-WP_002884726.1, NZ_KK737549.1, 418405, 419949, +, 1545, 4927055; cds-WP_002884725.1, NZ_KK737549.1, 419964, 420854, +, 891, 684378; cds-WP_002884721.1, NZ_KK737549.1, 421046, 421456, -, 411, 12674; cds-WP_013815099.1-65, NZ_KK737549.1, 421813, 422781, -, 969, 90331; cds-WP_021441021.1, NZ_KK737549.1, 422896, 424173, +, 1278, 81857; cds-AF52_RS04955, NZ_KK737549.1, 424257, 424484, +, 228, 3965; cds-WP_013815099.1-66, NZ_KK737549.1, 424540, 425508, -, 969, 88884; cds-AF52_RS04945, NZ_KK737549.1, 425541, 425747, -, 207, 33; cds-WP_004177855.1, NZ_KK737549.1, 425768, 426088, -, 321, 0; cds-AF52_RS04935, NZ_KK737549.1, 426081, 426602, -, 522, 23; cds-WP_013815099.1-67, NZ_KK737549.1, 426636, 427604, +, 969, 96999; cds-AF52_RS0124460, NZ_KK737549.1, 427655, 427843, +, 189, 4185; cds-WP_004177856.1, NZ_KK737549.1, 427951, 428460, +, 510, 5728; cds-WP_019725463.1, NZ_KK737549.1, 428469, 428924, +, 456, 83521; cds-WP_002884631.1, NZ_KK737549.1, 428974, 430623, -, 1650, 2615975; cds-WP_004146591.1, NZ_KK737549.1, 431388, 432737, +, 1350, 203566; cds-WP_002884627.1, NZ_KK737549.1, 432876, 433805, +, 930, 4745; cds-WP_002884625.1, NZ_KK737549.1, 433922, 434194, +, 273, 41613; cds-WP_002884619.1, NZ_KK737549.1, 434191, 435066, -, 876, 604599; cds-WP_002884616.1, NZ_KK737549.1, 435416, 436363, +, 948, 9845; cds-WP_032430268.1, NZ_KK737549.1, 436434, 437237, +, 804, 35037; cds-WP_021441018.1, NZ_KK737549.1, 437247, 437654, +, 408, 4; cds-WP_032430269.1, NZ_KK737549.1, 437654, 438148, +, 495, 11843; cds-WP_002884608.1, NZ_KK737549.1, 438214, 439014, +, 801, 6; cds-WP_032428464.1, NZ_KK737549.1, 439026, 439850, +, 825, 11; cds-WP_021441016.1, NZ_KK737549.1, 439923, 441155, +, 1233, 4308; cds-WP_002884583.1, NZ_KK737549.1, 441148, 441957, +, 810, 60475; cds-WP_004177864.1, NZ_KK737549.1, 442045, 443676, -, 1632, 43931; cds-WP_015959249.1, NZ_KK737549.1, 443806, 447489, -, 3684, 819846; cds-WP_002884578.1, NZ_KK737549.1, 447684, 448514, +, 831, 298650; cds-WP_002884575.1, NZ_KK737549.1, 448564, 450348, -, 1785, 17140; cds-WP_004192658.1, NZ_KK737549.1, 450451, 451755, -, 1305, 50659; cds-WP_002884559.1, NZ_KK737549.1, 451835, 453436, -, 1602, 159445; cds-WP_004146576.1, NZ_KK737549.1, 453697, 454626, -, 930, 27365; cds-WP_004178258.1, NZ_KK737549.1, 454783, 455244, +, 462, 97808; cds-WP_032425377.1, NZ_KK737549.1, 462509, 464098, +, 1590, 915070; cds-WP_002884357.1, NZ_KK737549.1, 464114, 465409, +, 1296, 115760; cds-WP_032430271.1, NZ_KK737549.1, 465399, 466730, -, 1332, 20469; cds-WP_021441012.1, NZ_KK737549.1, 466717, 468111, -, 1395, 11111; cds-WP_002884346.1, NZ_KK737549.1, 468371, 468808, +, 438, 10948; cds-WP_002884344.1, NZ_KK737549.1, 468812, 469507, -, 696, 845667; cds-WP_002884342.1, NZ_KK737549.1, 469520, 469792, -, 273, 2549261; cds-WP_002884331.1, NZ_KK737549.1, 469979, 470569, -, 591, 451451; cds-WP_009485014.1, NZ_KK737549.1, 470612, 471283, -, 672, 192952; cds-WP_004178235.1, NZ_KK737549.1, 471296, 472360, -, 1065, 717037; cds-WP_002884327.1, NZ_KK737549.1, 472401, 473174, -, 774, 231025; cds-WP_002884324.1, NZ_KK737549.1, 473268, 473765, +, 498, 296492; cds-WP_004178230.1, NZ_KK737549.1, 474028, 475923, +, 1896, 519887; cds-WP_004152310.1, NZ_KK737549.1, 475923, 476558, +, 636, 162216; cds-WP_004177871.1, NZ_KK737549.1, 476551, 477306, +, 756, 42874; cds-WP_002884183.1, NZ_KK737549.1, 477290, 477490, +, 201, 648; cds-WP_004146302.1, NZ_KK737549.1, 477492, 478262, +, 771, 8910; cds-WP_004212852.1, NZ_KK737549.1, 478259, 479392, +, 1134, 165772; cds-WP_004178221.1, NZ_KK737549.1, 479709, 481241, +, 1533, 202961; cds-WP_002884150.1, NZ_KK737549.1, 481414, 481719, +, 306, 111267; cds-WP_004212847.1, NZ_KK737549.1, 481723, 482040, +, 318, 291667; cds-WP_004192681.1, NZ_KK737549.1, 482083, 483423, -, 1341, 65313; cds-WP_002884146.1, NZ_KK737549.1, 483824, 488047, -, 4224, 30752610; cds-WP_004901914.1, NZ_KK737549.1, 488124, 492152, -, 4029, 36371778; cds-WP_002884142.1, NZ_KK737549.1, 492477, 492842, -, 366, 15023714; cds-WP_001207203.1, NZ_KK737549.1, 492909, 493406, -, 498, 28604293; cds-WP_002884036.1, NZ_KK737549.1, 493820, 494524, -, 705, 20771520; cds-WP_002884034.1, NZ_KK737549.1, 494528, 494956, -, 429, 11326001; cds-WP_001287521.1, NZ_KK737549.1, 495113, 495658, -, 546, 548182; cds-WP_002883772.1, NZ_KK737549.1, 495660, 496043, -, 384, 646760; cds-WP_004174069.1-2, NZ_KK737549.1, 496380, 497564, -, 1185, 58638224; cds-WP_002883524.1, NZ_KK737549.1, 498550, 499500, +, 951, 539956; cds-WP_002883521.1, NZ_KK737549.1, 499526, 500488, -, 963, 252625; cds-WP_032430275.1, NZ_KK737549.1, 500485, 501513, -, 1029, 280045; cds-WP_002883453.1, NZ_KK737549.1, 509001, 509534, -, 534, 211155; cds-WP_002883450.1, NZ_KK737549.1, 509546, 510997, -, 1452, 455332; cds-WP_002883449.1, NZ_KK737549.1, 511036, 511650, -, 615, 528237; cds-WP_032430276.1, NZ_KK737549.1, 511650, 512981, -, 1332, 1829920; cds-
WP_032430277.1, NZ_KK737549.1, 513173, 515362, +, 2190, 4512192; cds-WP _004150456.1, NZ_KK737549.1, 515372, 516535, +, 1164, 1563949; cds-WP _002883448.1, NZ_KK737549.1, 516642, 517343, -, 702, 930364; cds-WP_004146456.1, NZ_KK737549.1, 517393, 518868, -, 1476, 2001970; cds-WP_002883430.1, NZ_KK737549.1, 519047, 519535, +, 489, 171563; cds-WP _004146458.1, NZ_KK737549.1, 519532, 520335, -, 804, 90926; cds-WP_002883427.1, NZ_KK737549.1, 520368, 521147, -, 780, 307953; cds-WP_002883426.1, NZ_KK737549.1, 521150, 521686, -, 537, 495898; cds-WP _002883425.1, NZ_KK737549.1, 521690, 521941, -, 252, 353414; cds-WP _004177924.1, NZ_KK737549.1, 522071, 523711, -, 1641, 911888; cds-WP _021441002.1, NZ_KK737549.1, 523708, 524313, -, 606, 266043; cds-WP_002883421.1, NZ_KK737549.1, 524327, 525082, -, 756, 590827; cds-WP _021441001.1, NZ_KK737549.1, 525153, 526601, -, 1449, 572531; cds-WP _004181652.1, NZ_KK737549.1, 526737, 527498, -, 762, 298899; cds-WP_002883417.1, NZ_KK737549.1, 527771, 528583, +, 813, 713032; cds-WP_021441000.1, NZ_KK737549.1, 528642, 530906, -, 2265, 12977; cds-WP _002883415.1, NZ_KK737549.1, 531154, 532107, +, 954, 39218; cds-WP _021440999.1, NZ_KK737549.1, 531995, 532894, -, 900, 161624; cds-WP _004146466.1, NZ_KK737549.1, 532983, 533783, -, 801, 695800; cds-WP _002883412.1, NZ_KK737549.1, 533828, 534820, -, 993, 563485; cds-WP_002883411.1, NZ_KK737549.1, 534930, 535550, +, 621, 189238; cds-WP_021440998.1, NZ_KK737549.1, 535713, 536633, +, 921, 69865; cds-WP _021440997.1, NZ_KK737549.1, 536679, 537299, -, 621, 168082; cds-WP _032430279.1, NZ_KK737549.1, 537361, 539187, -, 1827, 710872; cds-WP _002883408.1, NZ_KK737549.1, 539255, 540115, -, 861, 225749; cds-WP_002883404.1, NZ_KK737549.1, 540268, 540732, +, 465, 141018; cds-WP _002883402.1, NZ_KK737549.1, 540781, 541674, +, 894, 570077; cds-WP_002883400.1, NZ_KK737549.1, 541718, 542668, -, 951, 710333; cds-WP_002883398.1, NZ_KK737549.1, 543024, 545186, -, 2163, 558453; cds-WP _032430280.1, NZ_KK737549.1, 545250, 545966, -, 717, 54548; cds-WP _002883396.1, NZ_KK737549.1, 545966, 546868, -, 903, 442366; cds-WP_002883395.1, NZ_KK737549.1, 546865, 547572, -, 708, 105401; cds-WP _002883394.1, NZ_KK737549.1, 547569, 548393, -, 825, 86176; cds-WP _002883392.1, NZ_KK737549.1, 548429, 548632, -, 204, 14747; cds-WP _002883389.1, NZ_KK737549.1, 548779, 549099, +, 321, 70300; cds-WP_002883383.1, NZ_KK737549.1, 549233, 551785, -, 2553, 2026914; cds-WP _021440994.1, NZ_KK737549.1, 552143, 553084, +, 942, 72238; cds-WP _012737128.1, NZ_KK737549.1, 553081, 553821, +, 741, 455096; cds-WP _004150429.1, NZ_KK737549.1, 553843, 555024, +, 1182, 693573; cds-WP _004150428.1, NZ_KK737549.1, 555028, 556224, +, 1197, 308187; cds-WP_002883322.1, NZ_KK737549.1, 557224, 558609, -, 1386, 348110; cds-WP_004186366.1, NZ_KK737549.1, 558815, 559555, -, 741, 93331; cds-WP_032428460.1, NZ_KK737549.1, 559567, 560913, -, 1347, 44692; cds-WP_021440992.1, NZ_KK737549.1, 560910, 561986, -, 1077, 286733; cds-WP _002883312.1, NZ_KK737549.1, 561983, 563233, -, 1251, 251740; cds-WP_002883310.1, NZ_KK737549.1, 563235, 564365, -, 1131, 327100; cds-WP_004146507.1, NZ_KK737549.1, 564371, 565045, -, 675, 48960; cds-WP _002883307.1, NZ_KK737549.1, 565023, 565904, -, 882, 533483; cds-WP_002883303.1, NZ_KK737549.1, 565923, 566990, -, 1068, 1031305; cds-WP_021440991.1, NZ_KK737549.1, 566987, 568249, -, 1263, 1056318; cds-WP _002883297.1, NZ_KK737549.1, 568246, 569376, -, 1131, 373621; cds-WP _002883296.1, NZ_KK737549.1, 569433, 570479, -, 1047, 437172; cds-WP_002883295.1, NZ_KK737549.1, 570493, 571596, -, 1104, 576306; cds-WP_002883293.1, NZ_KK737549.1, 571837, 573096, -, 1260, 5057217; cds-WP_002883224.1, NZ_KK737549.1, 573427, 573756, -, 330, 1525146; cds-WP _002883222.1, NZ_KK737549.1, 574098, 575363, +, 1266, 2529857; cds-WP_072271788.1, NZ_KK737549.1, 575482, 576990, +, 1509, 510820; cds-WP _002883211.1, NZ_KK737549.1, 577010, 577936, +, 927, 94282; cds-WP _004181642.1, NZ_KK737549.1, 578067, 579065, +, 999, 273; cds-WP_002883185.1, NZ_KK737549.1, 579128, 579850, +, 723, 26302; cds-WP_032430283.1, NZ_KK737549.1, 579855, 581876, -, 2022, 221289; cds-WP_002883181.1, NZ_KK737549.1, 581989, 582270, +, 282, 10375; cds-WP_002883180.1, NZ_KK737549.1, 582322, 582603, +, 282, 58670; cds-WP_002883178.1, NZ_KK737549.1, 582807, 584282, -, 1476, 648621; cds-WP_002883176.1, NZ_KK737549.1, 584435, 585325, +, 891, 230771; cds-WP _002883174.1, NZ_KK737549.1, 585322, 586866, -, 1545, 24473; cds-WP _002883173.1, NZ_KK737549.1, 586869, 588719, -, 1851, 398149; cds-WP_032430284.1, NZ_KK737549.1, 588780, 589709, -, 930, 317689; cds-WP _002883170.1, NZ_KK737549.1, 589726, 589983, -, 258, 56658; cds-WP _012737117.1, NZ_KK737549.1, 589980, 591626, -, 1647, 150715; cds-WP_001311244.1, NZ_KK737549.1, 591767, 591865, -, 99, 4155; cds-WP _032430285.1, NZ_KK737549.1, 592220, 593740, +, 1521, 3436; cds-WP _004146520.1, NZ_KK737549.1, 593766, 594104, -, 339, 78707; cds-WP_004177958.1, NZ_KK737549.1, 594223, 595044, +, 822, 141113; cds-WP _032430287.1, NZ_KK737549.1, 603456, 604307, -, 852, 200506; cds-WP_004146288.1, NZ_KK737549.1, 604252, 606090, -, 1839, 846330; cds-WP _002883023.1, NZ_KK737549.1, 606457, 607557, +, 1101, 371532; cds-WP _002883021.1, NZ_KK737549.1, 607609, 607968, -, 360, 274916; cds-WP_004151856.1, NZ_KK737549.1, 607984, 608619, -, 636, 199526; cds-WP _002883016.1, NZ_KK737549.1, 608816, 610216, +, 1401, 3711467; cds-WP _002883015.1, NZ_KK737549.1, 610199, 611116, -, 918, 2648382; cds-WP _032430289.1, NZ_KK737549.1, 611375, 612748, -, 1374, 224302; cds-WP_004187843.1, NZ_KK737549.1, 612848, 613624, -, 777, 83418; cds-WP_004181637.1, NZ_KK737549.1, 613631, 614635, -, 1005, 80038; cds-WP _019705783.1, NZ_KK737549.1, 614749, 615900, +, 1152, 228800; cds-WP_004181636.1, NZ_KK737549.1, 616159, 618810, +, 2652, 912258; cds-WP _021440981.1, NZ_KK737549.1, 618854, 619504, -, 651, 91438; cds-WP _004220885.1, NZ_KK737549.1, 619529, 620515, +, 987, 29054; cds-WP _004177977.1, NZ_KK737549.1, 620721, 621383, +, 663, 16960; cds-WP_004146274.1, NZ_KK737549.1, 621438, 622541, +, 1104, 80137; cds-WP_004177983.1, NZ_KK737549.1, 622605, 623486, -, 882, 60472; cds-WP _021440979.1, NZ_KK737549.1, 623630, 624517, -, 888, 22672; cds-WP_021440978.1, NZ_KK737549.1, 624746, 625654, -, 909, 26842; cds-WP_021440977.1, NZ_KK737549.1, 625746, 626117, +, 372, 123; cds-WP _032428457.1, NZ_KK737549.1, 626155, 627711, -, 1557, 3169; cds-WP _013815099.1-68, NZ_KK737549.1, 627780, 628748, +, 969, 81501; cds-WP_004181629.1, NZ_KK737549.1, 628916, 630052, +, 1137, 42485; cds-WP _032430291.1, NZ_KK737549.1, 630027, 632459, -, 2433, 638865; cds-WP _004150388.1, NZ_KK737549.1, 632462, 633622, -, 1161, 182326; cds-WP _002882924.1, NZ_KK737549.1, 633890, 634207, +, 318, 85006; cds-WP_002882922.1, NZ_KK737549.1, 634309, 634521, -, 213, 471155; cds-WP_025710954.1, NZ_KK737549.1, 634774, 636969, +, 2196, 118525; cds-WP_002882921.1, NZ_KK737549.1, 637120, 638148, +, 1029, 433177; cds-WP_071600598.1, NZ_KK737549.1, 638242, 639210, +, 969, 764115; cds-WP _002882918.1, NZ_KK737549.1, 639302, 639832, +, 531, 715217; cds-WP _002882917.1, NZ_KK737549.1, 639842, 641176, +, 1335, 2039351; cds-WP _032430292.1, NZ_KK737549.1, 641246, 642166, +, 921, 278754; cds-WP _032430293.1, NZ_KK737549.1, 642259, 642744, +, 486, 113047; cds-WP _002882911.1, NZ_KK737549.1, 642806, 643768, -, 963, 1271029; cds-WP _002882907.1, NZ_KK737549.1, 643965, 644867, -, 903, 175006; cds-WP_002882903.1, NZ_KK737549.1, 645026, 645529, -, 504, 1407550; cds-WP_002882901.1, NZ_KK737549.1, 645679, 646377, +, 699, 608246; cds-WP _032430294.1, NZ_KK737549.1, 646374, 647747, +, 1374, 1189502; cds-WP_002882896.1, NZ_KK737549.1, 647829, 648503, -, 675, 61863; cds-WP_002882894.1, NZ_KK737549.1, 648576, 649196, -, 621, 5892802; cds-WP_032430295.1, NZ_KK737549.1, 649486, 650520, +, 1035, 9174; cds-WP _002882891.1, NZ_KK737549.1, 650553, 651398, -, 846, 22093; cds-WP_002882888.1, NZ_KK737549.1, 651472, 652308, -, 837, 11060; cds-WP_002882885.1, NZ_KK737549.1, 652619, 654085, +, 1467, 7; cds-WP _004150375.1, NZ_KK737549.1, 654082, 655341, +, 1260, 2; cds-WP_004187188.1, NZ_KK737549.1, 655491, 656321, +, 831, 513; cds-WP _002882880.1, NZ_KK737549.1, 656403, 657389, +, 987, 49; cds-WP_021440966.1, NZ_KK737549.1, 657535, 659046, +, 1512, 28249; cds-WP _004181621.1, NZ_KK737549.1, 659043, 660050, +, 1008, 1265; cds-WP _032430296.1, NZ_KK737549.1, 660047, 661051, +, 1005, 17336; cds-WP_002882871.1, NZ_KK737549.1, 661141, 662289, +, 1149, 1809; cds-WP _021440964.1, NZ_KK737549.1, 662286, 662600, +, 315, 13; cds-WP _004210464.1, NZ_KK737549.1, 662638, 663903, -, 1266, 208509; cds-WP_004178018.1, NZ_KK737549.1, 663945, 666068, -, 2124, 293082; cds-WP_021440963.1, NZ_KK737549.1, 666184, 667179, -, 996, 87687; cds-WP_002882865.1, NZ_KK737549.1, 667267, 667815, -, 549, 21471; cds-WP_032430297.1, NZ_KK737549.1, 667915, 668571, +, 657, 92; cds-WP _004146241.1, NZ_KK737549.1, 668571, 668894, +, 324, 0; cds-WP _002882830.1, NZ_KK737549.1, 668935, 669147, -, 213, 8300; cds-WP_032430298.1, NZ_KK737549.1, 669276, 670112, -, 837, 3246; cds-WP _085921749.1, NZ_KK737549.1, 670269, 673319, +, 3051, 8926725; cds-WP _002882822.1, NZ_KK737549.1, 673332, 674234, +, 903, 1006616; cds-WP_032430299.1, NZ_KK737549.1, 674231, 674866, +, 636, 1079641; cds-WP _004150358.1, NZ_KK737549.1, 674863, 675792, +, 930, 1006235; cds-WP_019705794.1, NZ_KK737549.1, 675966, 676349, -, 384, 70760; cds-WP _004150355.1, NZ_KK737549.1, 676349, 676591, -, 243, 17458; cds-WP_032430300.1, NZ_KK737549.1, 676796, 677713, +, 918, 246510; cds-WP_004178031.1, NZ_KK737549.1, 677728, 678669, -, 942, 390213; cds-WP _002882809.1, NZ_KK737549.1, 678714, 679151, -, 438, 13848; cds-WP_004146232.1, NZ_KK737549.1, 679148, 680008, -, 861, 174210; cds-WP_004151865.1, NZ_KK737549.1, 680002, 680601, -, 600, 385240; cds-WP _002882755.1, NZ_KK737549.1, 680737, 682560, -, 1824, 5152853; cds-WP_002882753.1, NZ_KK737549.1, 682939, 684348, +, 1410, 2047855; cds-WP_004146229.1, NZ_KK737549.1, 684537, 685586, +, 1050, 115909; cds-WP _002882749.1, NZ_KK737549.1, 685595, 687004, +, 1410, 300230; cds-WP _004152951.1, NZ_KK737549.1, 687115, 687225, +, 111, 3242; cds-WP _002882745.1, NZ_KK737549.1, 687261, 688634, -, 1374, 407027; cds-WP_002882743.1, NZ_KK737549.1, 688823, 689329, -, 507, 275564; cds-WP_002882734.1, NZ_KK737549.1, 689913, 690545, +, 633, 458917; cds-WP_002882729.1, NZ_KK737549.1, 690893, 693685, -, 2793, 751903; cds-WP_004150336.1, NZ_KK737549.1, 694082, 694981, +, 900, 497301; cds-WP_004146224.1, NZ_KK737549.1, 695030, 695653, -, 624, 615897; cds-WP _004187940.1, NZ_KK737549.1, 695681, 696667, -, 987, 424123; cds-WP_002882612.1, NZ_KK737549.1, 696743, 697012, -, 270, 47847; cds-WP_004178039.1, NZ_KK737549.1, 697084, 697665, +, 582, 36386; cds-WP _002882596.1, NZ_KK737549.1, 697662, 698168, +, 507, 34487; cds-WP_004150973.1, NZ_KK737550.1, 3504, 6077, -, 2574, 2371793; cds-WP_021440571.1, NZ_KK737550.1, 6207, 6938, -, 732, 128402; cds-WP_032428369.1, NZ_KK737550.1, 6935, 7915, -, 981, 442514; cds-WP _004145664.1, NZ_KK737550.1, 8047, 8784, +, 738, 1065870; cds-WP_002914111.1, NZ_KK737550.1, 9055, 9390, +, 336, 608517; cds-WP_100250063.1, NZ_KK737550.1, 9497, 9544, +, 48, 44554; cds-WP_021440574.1, NZ_KK737550.1, 9645, 10805, +, 1161, 376799; cds-WP_021440575.1, NZ_KK737550.1, 10802, 11674, -, 873, 411730; cds-WP_014907099.1, NZ_KK737550.1, 11737, 12858, -, 1122, 114212; cds-WP_002914114.1, NZ_KK737550.1, 12868, 13938, -, 1071, 61242; cds-WP_004174804.1, NZ_KK737550.1, 14281, 14790, +, 510, 69769; cds-WP_002914116.1, NZ_KK737550.1, 14783, 16006, +, 1224, 88010; cds-WP_004145671.1, NZ_KK737550.1, 16020, 16502, +, 483, 118173; cds-WP_023285466.1, NZ_KK737550.1, 16511,17881, -, 1371, 157907; cds-WP _002914119.1, NZ_KK737550.1, 17938, 18396, -, 459, 304091; cds-WP _002914145.1, NZ_KK737550.1, 18516, 18863, -, 348, 8306018; cds-WP_002914147.1, NZ_KK737550.1, 18903, 19670, -, 768, 22476640; cds-WP _004150977.1, NZ_KK737550.1, 19702, 20250, -, 549, 14306309; cds-WP _002914149.1, NZ_KK737550.1, 20269, 20517, -, 249, 3348707; cds-WP _002914152.1, NZ_KK737550.1, 20777, 22141, -, 1365, 1032048; cds-WP_002914153.1, NZ_KK737550.1, 22305, 23096, +, 792, 144580; cds-WP_004149392.1, NZ_KK737550.1, 23116, 24402, +, 1287, 198871; cds-WP_004174800.1, NZ_KK737550.1, 24523, 25113, -, 591, 536112; cds-
WP_002914158.1, NZ_KK737550.1, 25238, 26116, +, 879, 175738; cds-WP_004174799.1, NZ_KK737550.1, 26203, 27864, +, 1662, 1814393; cds-WP _002914160.1, NZ_KK737550.1, 28012, 28353, +, 342, 733724; cds-WP _004145682.1, NZ_KK737550.1, 28420, 28710, -, 291, 20632; cds-WP _015875096.1, NZ_KK737550.1, 28700, 29176, -, 477, 62593; cds-WP _002914164.1, NZ_KK737550.1, 29287, 29769, +, 483, 982168; cds-WP _032430315.1, NZ_KK737550.1, 30546, 30794, +, 249, 67946; cds-WP _032430316.1, NZ_KK737550.1, 31673, 32251, -, 579, 269; cds-WP _032430369.1, NZ_KK737550.1, 32977, 34431, +, 1455, 4447; cds-WP _032430317.1, NZ_KK737550.1, 34432, 35181, +, 750, 1134; cds-WP _032430318.1, NZ_KK737550.1, 35321, 36064, -, 744, 612; cds-WP_032430320.1, NZ_KK737550.1, 36079, 37839, -, 1761, 142315; cds-AF52_RS03525, NZ_KK737550.1, 37916, 38458, -, 543, 2; cds-WP_013815099.1-69, NZ_KK737550.1, 38493, 39461, +, 969, 86319; cds-AF52_RS0124535, NZ_KK737550.1, 39512, 39685, +, 174, 15827; cds-WP _002914289.1, NZ_KK737550.1, 39771, 40220, -, 450, 107121; cds-WP_004217497.1, NZ_KK737550.1, 40931, 41350, +, 420, 23961; cds-WP _002914284.1, NZ_KK737550.1, 41423, 41602, -, 180, 49982; cds-AF52_RS03500, NZ_KK737550.1, 41808, 42377, +, 570, 245730; cds-WP_013815099.1-70, NZ_KK737550.1, 42433, 43401, -, 969, 119004; cds-AF52_RS03490, NZ_KK737550.1, 43435, 43776, +, 342, 46914; cds-WP _021440595.1, NZ_KK737550.1, 43757, 44302, -, 546, 91293; cds-WP _002914277.1, NZ_KK737550.1, 44310, 44609, -, 300, 41145; cds-WP _004212889.1, NZ_KK737550.1, 44685, 45290, -, 606, 382; cds-WP_021440594.1, NZ_KK737550.1, 45394, 46302, +, 909, 19156; cds-WP _021440593.1, NZ_KK737550.1, 46385, 48172, -, 1788, 262035; cds-WP _004174747.1, NZ_KK737550.1, 48436, 49956, +, 1521, 385083; cds-WP _013815099.1-71, NZ_KK737550.1, 50334, 51302, +, 969, 103547; cds-WP _032430322.1, NZ_KK737550.1, 51339, 51674, +, 336, 12471; cds-WP _021440597.1, NZ_KK737550.1, 51818, 53170, -, 1353, 31291; cds-WP _004151037.1, NZ_KK737550.1, 53259, 53690, +, 432, 92787; cds-WP_002914320.1, NZ_KK737550.1, 53874, 54119, +, 246, 8683; cds-WP _002914321.1, NZ_KK737550.1, 54116, 54526, +, 411, 14323; cds-WP_032428382.1, NZ_KK737550.1, 54499, 56640, +, 2142, 30271; cds-WP _021440599.1, NZ_KK737550.1, 56651, 57613, +, 963, 23715; cds-WP _002914328.1, NZ_KK737550.1, 57970, 59172, +, 1203, 2954; cds-WP_021312381.1, NZ_KK737550.1, 59165, 60232, +, 1068, 280; cds-WP _002914330.1, NZ_KK737550.1, 60311, 61309, +, 999, 94330; cds-WP_002914333.1, NZ_KK737550.1, 61487, 62233, +, 747, 28085; cds-WP _021440601.1, NZ_KK737550.1, 62223, 62558, +, 336, 4422; cds-WP_002914337.1, NZ_KK737550.1, 62649, 63179, +, 531, 316895; cds-WP_002914339.1, NZ_KK737550.1, 63305, 64477, +, 1173, 172156; cds-WP _002914342.1, NZ_KK737550.1, 64493, 66031, +, 1539, 401536; cds-WP _004185353.1, NZ_KK737550.1, 66168, 67844, +, 1677, 14585; cds-WP_004149452.1, NZ_KK737550.1, 67823, 68590, -, 768, 61950; cds-WP _002914351.1, NZ_KK737550.1, 68643, 69158, -, 516, 629506; cds-WP _002914353.1, NZ_KK737550.1, 69315, 70871, -, 1557, 1108268; cds-WP _002914355.1, NZ_KK737550.1, 70938, 71366, -, 429, 140128; cds-WP _004174701.1, NZ_KK737550.1, 71363, 71929, -, 567, 219589; cds-WP _002914359.1, NZ_KK737550.1, 72055, 72369, -, 315, 230879; cds-WP _000906486.1, NZ_KK737550.1, 73958, 74143, -, 186, 498425; cds-WP _004174700.1, NZ_KK737550.1, 74507, 77134, -, 2628, 4557005; cds-WP _004213244.1, NZ_KK737550.1, 77385, 77885, -, 501, 84805; cds-WP _002914769.1, NZ_KK737550.1, 77953, 79011, -, 1059, 4693558; cds-WP _002914771.1, NZ_KK737550.1, 79102, 79599, -, 498, 76488; cds-WP_032428383.1, NZ_KK737550.1, 79739, 80617, -, 879, 1372; cds-WP _004149458.1, NZ_KK737550.1, 80625, 81488, -, 864, 2; cds-AF52_RS0124565, NZ_KK737550.1, 81485, 82195, -, 711, 0; cds-WP _004174694.1, NZ_KK737550.1, 82534, 83613, -, 1080, 169202; cds-WP_002914812.1, NZ_KK737550.1, 83890, 84453, +, 564, 37108; cds-WP _021440607.1, NZ_KK737550.1, 84450, 85430, +, 981, 152838; cds-WP _004174693.1, NZ_KK737550.1, 85444, 85806, +, 363, 12421; cds-WP _002914815.1, NZ_KK737550.1, 85819, 86598, +, 780, 190488; cds-WP_021440608.1, NZ_KK737550.1, 86756, 87115, +, 360, 1686; cds-WP _002914817.1, NZ_KK737550.1, 87182, 87955, +, 774, 368945; cds-WP _004174691.1, NZ_KK737550.1, 87948, 88913, +, 966, 295086; cds-WP_004890240.1, NZ_KK737550.1, 88876, 90426, -, 1551, 14003; cds-WP _004145759.1, NZ_KK737550.1, 90614, 92062, +, 1449, 11249; cds-WP _021440610.1, NZ_KK737550.1, 92059, 93192, +, 1134, 1828; cds-WP _004181028.1, NZ_KK737550.1, 93289, 94302, -, 1014, 5630; cds-WP_032430324.1, NZ_KK737550.1, 94299, 96515, -, 2217, 919; cds-WP_002914875.1, NZ_KK737550.1, 96524, 97051, -, 528, 94; cds-WP _004174685.1, NZ_KK737550.1, 97188, 98201, -, 1014, 117797; cds-WP _032430325.1, NZ_KK737550.1, 98469, 99923, +, 1455, 46292; cds-WP _004181034.1, NZ_KK737550.1, 99938, 101362, +, 1425, 136268; cds-WP_002914883.1, NZ_KK737550.1, 101544, 101852, +, 309, 16050; cds-WP _032430326.1, NZ_KK737550.1, 101849, 102319, -, 471, 38966; cds-WP_002914887.1, NZ_KK737550.1, 102312, 102722, -, 411, 14988; cds-WP _002914891.1, NZ_KK737550.1, 102719, 103486, -, 768, 12766; cds-WP_002914893.1, NZ_KK737550.1, 103486, 104028, -, 543, 1157; cds-WP_021440614.1, NZ_KK737550.1, 104038, 105747, -, 1710, 155; cds-WP_032428387.1, NZ_KK737550.1, 105764, 106687, -, 924, 1940; cds-WP_004890213.1, NZ_KK737550.1, 106690, 108516, -, 1827, 0; cds-WP _004151047.1, NZ_KK737550.1, 108513, 109121, -, 609, 52; cds-WP _002914925.1, NZ_KK737550.1, 109204, 109653, -, 450, 0; cds-WP_002914928.1, NZ_KK737550.1, 109863, 110207, +, 345, 47; cds-WP_004181043.1, NZ_KK737550.1, 110211, 111083, +, 873, 394; cds-WP_014907033.1, NZ_KK737550.1, 111074, 111346, +, 273, 0; cds-WP _004145781.1, NZ_KK737550.1, 111346, 112467, +, 1122, 1529; cds-WP_032428388.1, NZ_KK737550.1, 112464, 113474, +, 1011, 94; cds-WP _032428389.1, NZ_KK737550.1, 113711, 115783, +, 2073, 78007; cds-WP _002914970.1, NZ_KK737550.1, 115837, 117237, -, 1401, 85715; cds-WP_021440618.1, NZ _KK737550.1, 117288, 119480, -, 2193,5647; cds-AF52_RS03110, NZ_KK737550.1, 119493, 120829, -, 1337, 267; cds-WP_032430327.1, NZ_KK737550.1, 121092, 122363, -, 1272, 111390; cds-WP_002914979.1, NZ _KK737550.1, 122541, 122855, +, 315, 7694; cds-WP _002914981.1, NZ_KK737550.1, 122865, 124187, +, 1323, 20667; cds-WP_032430328.1, NZ_KK737550.1, 124211, 124525, +, 315, 77; cds-WP _004151053.1, NZ_KK737550.1, 124576, 125490, +, 915, 107; cds-WP_004181055.1, NZ_KK737550.1, 125547, 126509, +, 963, 26870; cds-WP_004174672.1, NZ_KK737550.1, 126688, 127605, +, 918, 147849; cds-WP_021440623.1, NZ_KK737550.1, 127602, 128423, +, 822, 19193; cds-WP_004185394.1, NZ_KK737550.1, 128420, 129268, +, 849, 31738; cds-WP_002915035.1, NZ_KK737550.1, 129262, 130116, +, 855, 40108; cds-WP _004193741.1, NZ_KK737550.1, 130183, 130524, -, 342, 535; cds-WP_032430329.1, NZ_KK737550.1, 130591, 131478, -, 888, 51956; cds-WP _002915041.1, NZ_KK737550.1, 131663, 132604, +, 942, 2028; cds-WP_032430330.1, NZ_KK737550.1, 132640, 134349, +, 1710, 24974; cds-WP_021440627.1, NZ_KK737550.1, 134389, 134682, -, 294, 42018; cds-WP _021440628.1, NZ_KK737550.1, 134898, 137261, +, 2364, 43462; cds-WP _032428391.1, NZ_KK737550.1, 137333, 138364, +, 1032, 237194; cds-WP _004181072.1, NZ_KK737550.1, 138361, 139179, +, 819, 0; cds-WP_032428392.1, NZ_KK737550.1, 139179, 140174, +, 996, 80; cds-WP_021440630.1, NZ_KK737550.1, 140167, 140946, +, 780, 652; cds-WP _032430331.1, NZ_KK737550.1, 141100, 143661, +, 2562, 607588; cds-WP_021440632.1, NZ_KK737550.1, 143781, 143933, -, 153, 27882; cds-WP_002915098.1, NZ_KK737550.1, 144272, 144508, -, 237, 10370; cds-WP_004151060.1, NZ_KK737550.1, 144519, 145946, -, 1428, 22710; cds-WP _002915099.1, NZ_KK737550.1, 145946, 146539, -, 594, 1513; cds-WP_002915102.1, NZ_KK737550.1, 146713, 147120, +, 408, 3725; cds-WP_032430332.1, NZ_KK737550.1, 147129, 148016, -, 888, 46158; cds-WP_021440634.1, NZ_KK737550.1, 148125, 149327, +, 1203, 397; cds-WP _021440635.1, NZ_KK737550.1, 149324, 149701, +, 378, 3120; cds-WP _002915106.1, NZ_KK737550.1, 149754, 150746, -, 993, 1204836; cds-WP_014907019.1, NZ_KK737550.1, 150904, 152034, -, 1131, 2942188; cds-WP _002915108.1, NZ_KK737550.1, 152160, 152786, -, 627, 429204; cds-WP _004142750.1, NZ_KK737550.1, 152780, 153541, -, 762, 452973; cds-WP _004185423.1, NZ_KK737550.1, 153522, 154571, -, 1050, 228122; cds-WP_002915113.1, NZ_KK737550.1, 154568, 155047, -, 480, 150799; cds-WP _021440636.1, NZ_KK737550.1, 155047, 155757, -, 711, 360099; cds-WP_002915156.1, NZ_KK737550.1, 155777, 156094, -, 318, 416389; cds-WP_002915157.1, NZ_KK737550.1, 156235, 156561, -, 327, 21852; cds-WP_002915158.1, NZ_KK737550.1, 156612, 157217, -, 606, 6314; cds-WP_004181094.1, NZ_KK737550.1, 157217, 158644, -, 1428, 14975; cds-WP_002915160.1, NZ_KK737550.1, 158654, 159562, -, 909, 41571; cds-WP_072271746.1, NZ_KK737550.1, 159572, 160978, -, 1407, 14295; cds-WP_004174643.1, NZ_KK737550.1, 161225, 162271, +, 1047, 107673; cds-WP _002915177.1, NZ_KK737550.1, 162344, 163078, -, 735, 110119; cds-WP _021440639.1, NZ_KK737550.1, 163177, 164889, -, 1713, 76359; cds-WP_004174642.1, NZ_KK737550.1, 164889, 166688, -, 1800, 10298; cds-WP_187079196.1, NZ_KK737550.1, 167005, 167370, +, 366, 68444; cds-WP _021440641.1, NZ_KK737550.1, 167411, 168082, -, 672, 84773; cds-WP_032430333.1, NZ_KK737550.1, 168557, 169666, +, 1110, 204082; cds-WP_002915213.1, NZ_KK737550.1, 169730, 171028, -, 1299, 14397199; cds-WP _004142808.1, NZ_KK737550.1, 171110, 172747, -, 1638, 4025716; cds-WP _002915215.1, NZ_KK737550.1, 173028, 173819, -, 792, 109376; cds-WP_002915217.1, NZ_KK737550.1, 173931, 176168, -, 2238, 1188878; cds-WP_023301525.1, NZ_KK737550.1, 176290, 177594, -, 1305, 265875; cds-WP_004151066.1, NZ_KK737550.1, 177708, 180458, +, 2751, 345845; cds-WP _004149562.1, NZ_KK737550.1, 180616, 181755, -, 1140, 264950; cds-WP_002915223.1, NZ_KK737550.1, 181828, 183168, -, 1341, 445380; cds-WP _032428394.1, NZ_KK737550.1, 183186, 184526, -, 1341, 361447; cds-WP _002915225.1, NZ_KK737550.1, 184529, 185881, -, 1353, 895462; cds-WP_002915235.1, NZ_KK737550.1, 186353, 186796, -, 444, 81130; cds-WP _002915236.1, NZ_KK737550.1, 186801, 187595, -, 795, 128659; cds-WP _002915249.1, NZ_KK737550.1, 187595, 187924, -, 330, 37775; cds-WP_002915253.1, NZ_KK737550.1, 188556, 189101, -, 546, 177257; cds-WP_004174633.1, NZ_KK737550.1, 189170, 190015, +, 846, 262956; cds-WP_002915258.1, NZ_KK737550.1, 190127, 191494, +, 1368, 1334915; cds-WP _004151070.1, NZ_KK737550.1, 192004, 193293, +, 1290, 19709321; cds-WP_002915259.1, NZ_KK737550.1, 193359, 194726, +, 1368, 4967824; cds-WP_032430336.1, NZ_KK737550.1, 194865, 195806, +, 942, 318533; cds-WP _002915263.1, NZ_KK737550.1, 195852, 197075, -, 1224, 11722; cds-WP _004151071.1, NZ_KK737550.1, 197086, 198507, -, 1422, 1497; cds-WP_002915265.1, NZ_KK737550.1, 198701, 199579, -, 879, 55; cds-WP _002915267.1, NZ_KK737550.1, 199576, 200274, -, 699, 35; cds-WP _004174621.1, NZ_KK737550.1, 200271, 201293, -, 1023, 0; cds-WP _032428395.1, NZ_KK737550.1, 201290, 202696, -, 1407, 0; cds-AF52_RS0124590, NZ_KK737550.1, 202770, 204167, -, 1398, 0; cds-WP_021440649.1, NZ_KK737550.1, 204214, 205386, -, 1173, 4336; cds-WP_032428396.1, NZ_KK737550.1, 205464, 205580, -, 117, 0; cds-WP _021440650.1, NZ_KK737550.1, 205658, 207601, +, 1944, 2591; cds-WP _004142871.1, NZ_KK737550.1, 207698, 208453, +, 756, 21341; cds-WP _032430337.1, NZ_KK737550.1, 208512, 209660, -, 1149, 171739; cds-WP_002915470.1, NZ_KK737550.1, 209688, 210335, -, 648, 13582; cds-WP _004151076.1, NZ_KK737550.1, 210902, 212212, +, 1311, 11273; cds-WP _032430338.1, NZ_KK737550.1, 212245, 214020, +, 1776, 19495; cds-WP_004181132.1, NZ_KK737550.1, 214128, 215546, +, 1419, 3; cds-WP_002915538.1, NZ_KK737550.1, 215548, 215970, +, 423, 0; cds-WP_004142883.1, NZ_KK737550.1, 216029, 216739, +, 711, 67566; cds-WP_002915541.1, NZ_KK737550.1, 216788, 217888, -, 1101, 195631; cds-WP _002915543.1, NZ_KK737550.1, 217881, 218276, -, 396, 82615; cds-WP_002915545.1, NZ_KK737550.1, 218296, 219213, -, 918, 370533; cds-WP_002915549.1, NZ_KK737550.1, 219565, 219789, -, 225, 169286; cds-WP _021440652.1, NZ_KK737550.1, 219987, 221192, +, 1206, 122964; cds-WP_021440653.1, NZ_KK737550.1, 221192, 221623, +, 432, 40034; cds-WP_002915577.1, NZ_KK737550.1, 221666, 222487, -, 822, 78300; cds-WP_002915579.1, NZ_KK737550.1, 222575, 223672, -, 1098, 290044; cds-WP _004900253.1, NZ_KK737550.1, 224055, 225110, -, 1056, 75348; cds-WP 004143921.1, NZ_KK737550.1, 225154, 225699, -, 546, 847; cds-WP_021440655.1, NZ_KK737550.1, 225693, 227216, -
1524, 22678; cds-WP_002915614.1, NZ_KK737550.1, 227213, 228037, -, 825, 16311; cds-WP_004143925.1, NZ_KK737550.1, 228046, 228723, -, 678, 33790; cds-WP_002915619.1, NZ_KK737550.1, 228710, 228991, -, 282, 846; cds-WP_004143926.1, NZ_KK737550.1, 228993, 229730, -, 738, 29425; cds-WP_004143927.1, NZ_KK737550.1, 229730, 230440, -, 711, 15699; cds-WP_016532596.1, NZ_KK737550.1, 230437, 231231, -, 795, 10787; cds-WP_002915627.1, NZ_KK737550.1, 231242, 232027, -, 786, 27225; cds-WP_004143930.1, NZ_KK737550.1, 232024, 232749, -, 726, 13293; cds-WP_021440657.1, NZ_KK737550.1, 232749, 233804, -, 1056, 48609; cds-WP_004149602.1, NZ_KK737550.1, 233785, 234558, -, 774, 61417; cds-WP_032430341.1, NZ_KK737550.1, 234551, 235120, -, 570, 1087; cds-WP_016947545.1, NZ_KK737550.1, 235110, 235715, -, 606, 439; cds-WP_002915708.1, NZ_KK737550.1, 235709, 236848, -, 1140, 19776; cds-WP_021440659.1, NZ_KK737550.1, 236845, 237480, -, 636, 417; cds-WP_002915724.1, NZ_KK737550.1, 237491, 238450, -, 960, 3265; cds-WP_004185466.1, NZ_KK737550.1, 238447, 239823, -, 1377, 95783; cds-AF52_RS02545, NZ_KK737550.1, 240376, 241203, -, 828, 6296; cds-AF52_RS26545, NZ_KK737550.1, 241259, 241436, -, 178, 5379; cds-WP_170999280.1, NZ_KK737550.1, 241947, 242236, -, 290, 16897; cds-WP_002915737.1, NZ_KK737550.1, 242699, 242983, +, 285, 0; cds-WP_002915739.1, NZ_KK737550.1, 242980, 243792, +, 813, 0; cds-WP_002915744.1, NZ_KK737550.1, 243811, 245475, +, 1665, 38; cds-WP_008806302.1, NZ_KK737550.1, 245486, 246175, +, 690, 460; cds-WP_021440661.1, NZ_KK737550.1, 246190, 246714, +, 525, 1; cds-WP_021440662.1, NZ_KK737550.1, 246727, 248559, +, 1833, 2; cds-WP_004900279.1, NZ_KK737550.1, 248549, 248899, +, 351, 0; cds-WP_002915806.1, NZ_KK737550.1, 248918, 249193, +, 276, 0; cds-WP_002915808.1, NZ_KK737550.1, 249215, 249661, +, 447, 82; cds-WP_002915810.1, NZ_KK737550.1, 249661, 250293, +, 633, 221; cds-WP_021440663.1, NZ_KK737550.1, 250290, 250781, +, 492, 19; cds-WP_021440664.1, NZ_KK737550.1, 250785, 251060, +, 276, 0; cds-WP_004211864.1, NZ_KK737550.1, 251071, 252087, +, 1017, 375; cds-WP_021440665.1, NZ_KK737550.1, 252084, 253472, +, 1389, 747; cds-WP_021440666.1, NZ_KK737550.1, 253484, 254596, +, 1113, 10035; cds-WP_023301518.1, NZ_KK737550.1, 254593, 255945, +, 1353, 0; cds-WP_004181171.1, NZ_KK737550.1, 255948, 256502, +, 555, 866; cds-WP_002915850.1, NZ_KK737550.1, 256502, 256852, +, 351, 3049; cds-WP_004185483.1, NZ_KK737550.1, 256857, 257297, +, 441, 13529; cds-WP_032430342.1, NZ_KK737550.1, 257294, 258508, +, 1215, 15192; cds-WP_032428406.1, NZ_KK737550.1, 258600, 259466, +, 867, 258189; cds-WP_004143954.1, NZ_KK737550.1, 259470, 260546, -, 1077, 99124; cds-WP_004174538.1, NZ_KK737550.1, 260564, 261817, -, 1254, 281531; cds-AF52_RS02415, NZ_KK737550.1, 262045, 263376, +, 1332, 31299; cds-WP_009485668.1, NZ_KK737550.1, 263606, 263926, +, 321, 3151; cds-WP_004900323.1, NZ_KK737550.1, 263984, 265828, -, 1845, 15585; cds-WP_021440669.1, NZ_KK737550.1, 265825, 269361, -, 3537, 352293; cds-WP_023301515.1, NZ_KK737550.1, 269358, 272243, -, 2886, 356943; cds-WP_004211885.1, NZ_KK737550.1, 272364, 275741, -, 3378, 284553; cds-WP_021440672.1, NZ_KK737550.1, 275754, 276077, -, 324, 4661; cds-WP_004181182.1, NZ_KK737550.1, 276062, 276469, -, 408, 4; cds-WP_021440673.1, NZ_KK737550.1, 276466, 277023, -, 558, 1781; cds-WP_002915932.1, NZ_KK737550.1, 277020, 277571, -, 552, 5954; cds-WP_002915933.1, NZ_KK737550.1, 277757, 278551, -, 795, 659423; cds-WP_002915934.1, NZ_KK737550.1, 278558, 279433, -, 876, 630628; cds-WP_032430343.1, NZ_KK737550.1, 279679, 281925, -, 2247,924308; cds-WP_002915936.1, NZ_KK737550.1, 281938, 282468, -, 531, 331577; cds-WP_004218186.1, NZ_KK737550.1, 282922, 283065, +, 144, 0; cds-WP_004143967.1, NZ_KK737550.1, 283151, 283846, +, 696, 19039; cds-WP_002915973.1, NZ_KK737550.1, 283910, 284623, +, 714, 125265; cds-WP_002915974.1, NZ_KK737550.1, 284747, 284965, +, 219, 150253; cds-WP_032430346.1, NZ_KK737550.1, 285186, 286226, +, 1041, 256289; cds-WP_004185495.1, NZ_KK737550.1, 286329, 287522, -, 1194, 114152; cds-WP_002915977.1, NZ_KK737550.1, 287515, 289674, -, 2160, 398432; cds-AF52_RS28115, NZ_KK737550.1, 289938, 290202, -, 265, 4096; cds-WP_002915981.1, NZ_KK737550.1, 290296, 291312, +, 1017, 910505; cds-WP_021440677.1, NZ_KK737550.1, 291273, 291752, -, 480, 19842; cds-WP_032430347.1, NZ_KK737550.1, 291749, 292522, -, 774, 348389; cds-WP_004174521.1, NZ_KK737550.1, 292590, 294017, -, 1428, 19375; cds-WP_004174520.1, NZ_KK737550.1, 294025, 295362, -, 1338, 43389; cds-WP_021440678.1, NZ_KK737550.1, 295662, 296663, +, 1002, 1489810; cds-WP_002915990.1, NZ_KK737550.1, 296656, 297918, -, 1263, 259772; cds-WP_002915991.1, NZ_KK737550.1, 298055, 298981, +, 927, 21796; cds-WP_032428400.1, NZ_KK737550.1, 298947, 299660, -, 714, 13916; cds-WP_002915993.1, NZ_KK737550.1, 299800, 300126, +, 327, 43099; cds-WP_002915995.1, NZ_KK737550.1, 300579, 301469, +, 891, 4870; cds-WP_002915996.1, NZ_KK737550.1, 301462, 302364, +, 903, 12674; cds-WP_021440680.1, NZ_KK737550.1, 302377, 303504, +, 1128, 85793; cds-WP_002915998.1, NZ_KK737550.1, 303521, 304807, +, 1287, 92047; cds-WP_032415517.1, NZ_KK737550.1, 304867, 306210, -, 1344, 6399; cds-WP_026005853.1, NZ_KK737550.1, 306332, 307765, -, 1434, 20051; cds-WP_002916000.1, NZ_KK737550.1, 307785, 309026, -, 1242, 6356; cds-WP_004143987.1, NZ_KK737550.1, 309039, 309173, -, 135, 7493; cds-WP_032428401.1, NZ_KK737550.1, 309268, 310263, +, 996, 174589; cds-WP_002916001.1, NZ_KK737550.1, 310260, 311681, -, 1422, 8169; cds-WP_002916003.1, NZ_KK737550.1, 311977, 312738, -, 762, 22841; cds-WP_002916048.1, NZ_KK737550.1, 313224, 314399, +, 1176, 27248; cds-WP_004143995.1, NZ_KK737550.1, 314535, 315230, -, 696, 9789; cds-WP_002916052.1, NZ_KK737550.1, 315479, 316657, -, 1179, 272462; cds-WP_002916055.1, NZ_KK737550.1, 316776, 317645, -, 870, 2870; cds-WP_002916056.1, NZ_KK737550.1, 317748, 318209, +, 462, 8022; cds-WP_002916058.1, NZ_KK737550.1, 318333, 319562, +, 1230, 121470; cds-WP_002916061.1, NZ_KK737550.1, 319681, 320559, +, 879, 28629; cds-WP_013815099.1-72, NZ_KK737550.1, 320898, 321866, -, 969, 75772; cds-AF52_RS02175, NZ_KK737550.1, 321899, 322303, +, 405, 1154; cds-WP_013815099.1-73, NZ_KK737550.1, 322616, 323584, +, 969, 81641; cds-WP_021440689.1, NZ_KK737550.1, 323835, 324443, +, 609, 157577; cds-WP_002916190.1, NZ_KK737550.1, 324922, 325470, +, 549, 16219; cds-WP_021440690.1, NZ_KK737550.1, 325541, 326077, +, 537, 4544; cds-WP_002916192.1, NZ_KK737550.1, 326106, 326831, +, 726, 40058; cds-WP_032430351.1, NZ_KK737550.1, 326913, 329525, +, 2613, 1853; cds-WP_004185520.1, NZ_KK737550.1, 329536, 330063, +, 528, 1420; cds-WP_004229447.1, NZ_KK737550.1, 330091, 330576, +, 486, 60091; cds-WP_032423847.1, NZ_KK737550.1, 330594, 331496, +, 903, 8562; cds-WP_021440694.1, NZ_KK737550.1, 331493, 332905, +, 1413, 8400; cds-WP_002916201.1, NZ_KK737550.1, 332948, 333220, -, 273, 25172; cds-WP_004144031.1, NZ_KK737550.1, 333232, 333501, -, 270, 21995; cds-WP_002916204.1, NZ_KK737550.1, 333668, 333940, +, 273, 42983; cds-WP_004174502.1, NZ_KK737550.1, 333947, 334972, +, 1026, 208869; cds-WP_004185523.1, NZ_KK737550.1, 334985, 336922, +, 1938, 90733; cds-WP_032430352.1, NZ_KK737550.1, 336919, 338367, +, 1449, 78446; cds-WP_002916274.1, NZ_KK737550.1, 338632, 338931, -, 300, 25889; cds-WP_002916277.1, NZ_KK737550.1, 339116, 340669, -, 1554, 3965380; cds-WP_016947386.1, NZ_KK737550.1, 341142, 341564, -, 423, 273390; cds-WP_023301507.1, NZ_KK737550.1, 341574, 342866, -, 1293, 49690; cds-WP_002916281.1, NZ_KK737550.1, 342918, 343247, -, 330, 984; cds-WP_002916282.1, NZ_KK737550.1, 343466, 343759, +, 294, 92097; cds-WP_002916283.1, NZ_KK737550.1, 343790, 344362, +, 573, 73853; cds-WP_014599254.1, NZ_KK737550.1, 344441, 344644, +, 204, 7536; cds-WP_004174493.1, NZ_KK737550.1, 344813, 345712, -, 900, 262584; cds-WP_004151785.1, NZ_KK737550.1, 345851, 346159, +, 309, 3769; cds-WP_023285538.1, NZ_KK737550.1, 346168, 346725, +, 558, 2143; cds-WP_032428403.1, NZ_KK737550.1, 346746, 347765, +, 1020, 13013; cds-WP_004185540.1, NZ_KK737550.1, 347798, 348601, +, 804, 48831; cds-WP_015958948.1, NZ_KK737550.1, 348965, 349894, -, 930, 61350; cds-AF52_RS02025, NZ_KK737550.1, 350024, 351103, +, 1080, 211; cds-WP_013815099.1-74, NZ_KK737550.1, 351161, 352129, -, 969, 81068; cds-AF52_RS26135, NZ_KK737550.1, 352161, 352478, +, 318, 4756; cds-WP_004157874.1, NZ_KK737550.1, 352800, 353513, -, 714, 62273; cds-WP_002916298.1, NZ_KK737550.1, 353830, 354384, +, 555, 10495; cds-WP_002916299.1, NZ_KK737550.1, 354620, 356137, -, 1518, 2340499; cds-WP_095858446.1, NZ_KK737550.1;NZ_KK737550.1, 356147;357171, 357169;357245, -;-, 1098, 1434989; cds-WP_004149758.1, NZ_KK737550.1, 357331, 359064, -, 1734, 290570; cds-WP_004185548.1, NZ_KK737550.1, 359070, 359783, -, 714, 532805; cds-WP_004144729.1, NZ_KK737550.1, 359806, 360702, -, 897, 400869; cds-WP_004144730.1, NZ_KK737550.1, 360803, 361324, +, 522, 163681; cds-WP_002916310.1, NZ_KK737550.1, 361331, 361741, -, 411, 143261; cds-WP_002916312.1, NZ_KK737550.1, 361722, 361988, -, 267, 89776; cds-WP_004185555.1, NZ_KK737550.1, 362234, 363217, +, 984, 485976; cds-WP_002916317.1, NZ_KK737550.1, 363368, 364027, -, 660, 161030; cds-WP_004144734.1, NZ_KK737550.1, 364191, 364502, -, 312, 20647; cds-WP_002916321.1, NZ_KK737550.1, 364552, 365280, +, 729, 130891; cds-WP_002916322.1, NZ_KK737550.1, 365399, 366832, +, 1434, 69569; cds-WP_004185561.1, NZ_KK737550.1, 366957, 367316, +, 360, 123214; cds-WP_009308616.1, NZ_KK737550.1, 367366, 369375, +, 2010, 26477; cds-WP_009308617.1, NZ_KK737550.1, 369377, 369988, +, 612, 4264; cds-WP_009308618.1, NZ_KK737550.1, 369978, 370481, +, 504, 6544; cds-WP_004174442.1, NZ_KK737550.1, 370602, 371345, -, 744, 85386; cds-WP_004900541.1, NZ_KK737550.1, 371408, 374281, -, 2874, 9658006; cds-WP_002916479.1, NZ_KK737550.1, 374488, 374877, -, 390, 2205620; cds-WP_002916480.1, NZ_KK737550.1, 374902, 375996, -, 1095, 3161974; cds-WP_002916481.1, NZ_KK737550.1, 376419, 377621, -, 1203, 170190; cds-WP_021440702.1, NZ_KK737550.1, 377632, 378810, -, 1179, 348482; cds-WP_002916483.1, NZ_KK737550.1, 378807, 380123, -, 1317, 680264; cds-WP_002916486.1, NZ_KK737550.1, 380147, 380725, -, 579, 428520; cds-WP_002916488.1, NZ_KK737550.1, 380892, 381221, +, 330, 271983; cds-WP_004144746.1, NZ_KK737550.1, 381545, 382141, +, 597, 286285; cds-WP_002916493.1, NZ_KK737550.1, 382225, 383457, -, 1233, 356525; cds-WP_002916495.1, NZ_KK737550.1, 383727, 384386, -, 660, 245535; cds-WP_002916497.1, NZ_KK737550.1, 384554, 385447, +, 894, 51205; cds-WP_002916499.1, NZ_KK737550.1, 385501, 386268, -, 768, 788861; cds-WP_004144747.1, NZ_KK737550.1, 386358, 386993, -, 636, 50054; cds-WP_002916504.1, NZ_KK737550.1, 387145, 388002, -, 858, 412448; cds-WP_004144748.1, NZ_KK737550.1, 388193, 389272, -, 1080, 15821335; cds-WP_002916508.1, NZ_KK737550.1, 389370, 390533, -, 1164, 5218215; cds-WP_002916512.1, NZ_KK737550.1, 390573, 391601, -, 1029, 1957486; cds-WP_004174438.1, NZ_KK737550.1, 391987, 393978, -, 1992, 9200859; cds-WP_004151780.1, NZ_KK737550.1, 394257, 395015, +, 759, 271935; cds-WP_032428408.1, NZ_KK737550.1, 395393, 396661, +, 1269, 38133; cds-WP_021440705.1, NZ_KK737550.1, 396798, 398438, -, 1641, 18026; cds-WP_032430353.1, NZ_KK737550.1, 398452, 399921, -, 1470, 6; cds-WP_004211378.1, NZ_KK737550.1, 399937, 400701, -, 765, 0; cds-WP_032430354.1, NZ_KK737550.1, 400711, 401505, -, 795, 14; cds-WP_032428409.1, NZ_KK737550.1, 401489, 402259, -, 771, 10; cds-WP_032430355.1, NZ_KK737550.1, 402269, 402799, -, 531, 19209; cds-WP_004149797.1, NZ_KK737550.1, 402810, 404066, -, 1257, 1026; cds-WP_004174432.1, NZ_KK737550.1, 404093, 405289, -, 1197, 113055; cds-WP_004149799.1, NZ_KK737550.1, 405286, 406212, -, 927, 33563; cds-WP_021440707.1, NZ_KK737550.1, 406301, 407089, -, 789, 47017; cds-WP_021440708.1, NZ_KK737550.1, 407103, 407852, -, 750, 43637; cds-WP_004174429.1, NZ_KK737550.1, 407852, 408874, -, 1023, 322984; cds-WP_004144765.1, NZ_KK737550.1, 408885, 409193, -, 309, 204510; cds-WP_004151769.1, NZ_KK737550.1, 409190, 409513, -, 324, 39322; cds-WP_032428411.1, NZ_KK737550.1, 409941, 411161, +, 1221, 59797; cds-WP_002916572.1, NZ_KK737550.1, 411289, 412209, -, 921, 385322; cds-WP_004149805.1, NZ_KK737550.1, 412443, 414419, -, 1977, 1108596; cds-WP_002916578.1,
NZ_KK737550.1, 414428, 414559, -, 132, 81024; cds-WP_157828762.1, NZ_KK737550.1, 414740, 415063, +, 324, 25104; cds-WP_004149807.1, NZ_KK737550.1, 415261, 416415, +, 1155, 977597; cds-WP_002916587.1, NZ_KK737550.1, 416803, 418197, +, 1395, 488743; cds-WP_002916588.1, NZ_KK737550.1, 418275, 418775, +, 501, 157364; cds-WP_002916589.1, NZ_KK737550.1, 418870, 419577, +, 708, 84446; cds-WP_002916597.1, NZ_KK737550.1, 419669, 420400, +, 732, 333278; cds-WP_002916600.1, NZ_KK737550.1, 420421, 421371, +, 951, 1116852; cds-WP_002916603.1, NZ_KK737550.1, 421546, 422109, +, 564, 238850; cds-WP_002916605.1, NZ_KK737550.1, 422109, 422525, +, 417, 101346; cds-WP_002916607.1, NZ_KK737550.1, 422578, 423231, -, 654, 60467; cds-WP_004149811.1, NZ_KK737550.1, 423580, 424560, -, 981, 56; cds-WP_004144780.1, NZ_KK737550.1, 424577, 425278, +, 702, 215297; cds-WP_002916613.1, NZ_KK737550.1, 425299, 425865, +, 567, 327123; cds-WP_002916614.1, NZ_KK737550.1, 425862, 426152, +, 291, 300982; cds-WP_002916615.1, NZ_KK737550.1, 426165, 426758, +, 594, 289517; cds-WP_004149812.1, NZ_KK737550.1, 426751, 427887, +, 1137, 246125; cds-WP_023285550.1, NZ_KK737550.1, 428193, 429179, +, 987, 46035; cds-WP_025861936.1, NZ_KK737550.1, 429226, 429729, +, 504, 19990; cds-WP_004149816.1, NZ_KK737550.1, 429729, 431030, +, 1302, 65276; cds-WP_019704316.1, NZ_KK737550.1, 431078, 431797, -, 720, 265537; cds-WP_002916620.1, NZ_KK737550.1, 431848, 432174, -, 327, 36595; cds-WP_002916627.1, NZ_KK737550.1, 432174, 432893, -, 720, 170874; cds-AF52_RS01615, NZ_KK737550.1, 433030, 434087, +, 1058, 209600; cds-WP_004105744.1, NZ_KK737550.1, 434116, 434391, +, 276, 279194; cds-WP_004900580.1, NZ_KK737550.1, 434441, 435523, +, 1083, 966916; cds-WP_004144787.1, NZ_KK737550.1, 435715, 436968, +, 1254, 1160389; cds-WP_009484134.1, NZ_KK737550.1, 437012, 439150, -, 2139, 104886; cds-WP_002916694.1, NZ_KK737550.1, 439570, 440283, +, 714, 14819; cds-AF52_RS01580, NZ_KK737550.1, 441124, 441340, -, 217, 192384; cds-WP_004188553.1, NZ_KK737550.1, 441437, 442030, -, 594, 244638; cds-WP_021314147.1, NZ_KK737550.1, 442734, 443219, -, 486, 92408; cds-WP_004150912.1, NZ_KK737550.1, 443216, 443509, -, 294, 88136; cds-AF52_RS27585, NZ_KK737550.1, 444170, 444344, +, 175, 5874; cds-WP_032175380.1-2, NZ_KK737550.1, 445134, 445482, +, 349, 32321; cds-WP_004217775.1, NZ_KK737550.1, 445564, 446940, +, 1377, 23751; cds-WP_004150915.1, NZ_KK737550.1, 446951, 448099, +, 1149, 41910; cds-WP_004188550.1, NZ_KK737550.1, 448100, 449011, -, 912, 13467; cds-WP_021440720.1, NZ_KK737550.1, 449109, 449711, +, 603, 4266; cds-WP_004174409.1, NZ_KK737550.1, 449798, 450142, +, 345, 14753; cds-WP_002916738.1, NZ_KK737550.1, 450342, 450530, +, 189, 56099; cds-WP_004149825.1, NZ_KK737550.1, 450707, 451888, -, 1182, 236221; cds-WP_002916740.1, NZ_KK737550.1, 452034, 453899, -, 1866, 997092; cds-WP_004217169.1, NZ_KK737550.1, 453976, 454098, -, 123, 9456; cds-WP_004150918.1, NZ_KK737550.1, 454119, 454679, +, 561, 37924; cds-WP_002916742.1, NZ_KK737550.1, 454727, 455593, +, 867, 216859; cds-WP_002916743.1, NZ_KK737550.1, 455666, 455953, -, 288, 24138; cds-WP_004144560.1, NZ_KK737550.1, 456032, 456919, -, 888, 17490; cds-WP_004105804.1, NZ_KK737550.1, 457041, 457535, -, 495, 162125; cds-WP_032412422.1, NZ_KK737550.1, 457642, 459165, -, 1524, 94980; cds-WP_002916782.1, NZ_KK737550.1, 459179, 460597, -, 1419, 75010; cds-WP_002916785.1, NZ_KK737550.1, 460802, 461227, -, 426, 284669; cds-WP_004174395.1, NZ_KK737550.1, 461234, 461965, -, 732, 290065; cds-WP_004149836.1, NZ_KK737550.1, 462216, 463403, +, 1188, 766; cds-WP_002916791.1, NZ_KK737550.1, 463535, 464194, +, 660, 277066; cds-WP_032430356.1, NZ_KK737550.1, 464244, 465143, -, 900, 85309; cds-WP_004144551.1, NZ_KK737550.1, 465340, 466503, +, 1164, 34031; cds-WP_023286874.1, NZ_KK737550.1, 466649, 467476, +, 828, 171362; cds-WP_004144550.1, NZ_KK737550.1, 467512, 468039, -, 528, 77031; cds-WP_004174383.1, NZ_KK737550.1, 468096, 470279, -, 2184, 837501; cds-WP_004144547.1, NZ_KK737550.1, 470403, 471815, -, 1413, 415693; cds-WP_002916826.1, NZ_KK737550.1, 471899, 472636, -, 738, 399702; cds-WP_004181324.1, NZ_KK737550.1, 472828, 475086, -, 2259, 1160059; cds-WP_002916831.1, NZ_KK737550.1, 475208, 476077, -, 870, 100129; cds-WP_002916833.1, NZ_KK737550.1, 476155, 476550, -, 396, 34620; cds-WP_002916836.1, NZ_KK737550.1, 476701, 477360, +, 660, 43517; cds-WP_021440728.1, NZ_KK737550.1, 477357, 478706, +, 1350, 22063; cds-WP_002916840.1, NZ_KK737550.1, 478813, 479394, +, 582, 86188; cds-WP_002916842.1, NZ_KK737550.1, 479475, 479789, +, 315, 423699; cds-WP_002916844.1, NZ_KK737550.1, 479862, 481757, -, 1896, 1808341; cds-WP_002916845.1, NZ_KK737550.1, 481788, 482363, -, 576, 984898; cds-WP_002916846.1, NZ_KK737550.1, 482363, 483190, -, 828, 1334902; cds-WP_002916847.1, NZ_KK737550.1, 483215, 483637, -, 423, 335767; cds-WP_002916848.1, NZ_KK737550.1, 483634, 484266, -, 633, 69201; cds-WP_004150921.1, NZ_KK737550.1, 484463, 485932, +, 1470, 2236408; cds-WP_002916849.1, NZ_KK737550.1, 486100, 486771, +, 672, 829779; cds-WP_004900674.1, NZ_KK737550.1, 486777, 487937, +, 1161, 1983418; cds-WP_004174357.1, NZ_KK737550.1, 487982, 488773, -, 792, 272823; cds-WP_032430358.1, NZ_KK737550.1, 488960, 489730, +, 771, 31171; cds-WP_004185664.1, NZ_KK737550.1, 489792, 490445, -, 654, 239659; cds-WP_002916856.1, NZ_KK737550.1, 490823, 491095, +, 273, 11088; cds-WP_002916857.1, NZ_KK737550.1, 491131, 491328, -, 198, 15411; cds-AF52_RS28140, NZ_KK737550.1, 491320, 491470, +, 151, 5035; cds-WP_002916858.1, NZ_KK737550.1, 491548, 492981, -, 1434, 852788; cds-WP_002916860.1, NZ_KK737550.1, 493040, 495877, -, 2838, 1093939; cds-WP_009308672.1, NZ_KK737550.1, 495898, 497196, -, 1299, 33220; cds-WP_002916862.1, NZ_KK737550.1, 497411, 498031, +, 621, 539498; cds-WP_002916864.1, NZ_KK737550.1, 498093, 499334, +, 1242, 1314679; cds-WP_004144532.1, NZ_KK737550.1, 499346, 500167, -, 822, 506318; cds-WP_004150923.1, NZ_KK737550.1, 500366, 500749, -, 384, 190158; cds-WP_004144530.1, NZ_KK737550.1, 500857, 501474, +, 618, 105668; cds-WP_021440736.1, NZ_KK737550.1, 501606, 501725, +, 120, 0; cds-AF52_RS27590, NZ_KK737550.1, 501736, 501798, -, 63, 993; cds-WP_013815099.1-75, NZ_KK737550.1, 501856, 502824, -, 969, 76225; cds-WP_032430359.1, NZ_KK737550.1, 502826, 502948, -, 123, 1; cds-WP_004174349.1, NZ_KK737550.1, 502938, 503762, +, 825, 1842; cds-WP_002916871.1, NZ_KK737550.1, 503772, 504074, +, 303, 2299; cds-WP_021440738.1, NZ_KK737550.1, 504084, 504404, +, 321, 0; cds-WP_015958999.1, NZ_KK737550.1, 504397, 506100, +, 1704, 9110; cds-WP_004215466.1, NZ_KK737550.1, 506110, 506586, +, 477, 316; cds-WP_016532672.1, NZ_KK737550.1, 506588, 507262, +, 675, 302; cds-WP_002916877.1, NZ_KK737550.1, 507271, 507888, +, 618, 1335; cds-WP_025367880.1, NZ_KK737550.1, 508042, 509649, +, 1608, 93528; cds-WP_004900709.1, NZ_KK737550.1, 509694, 510707, -, 1014, 56676; cds-WP_001144069.1, NZ_KK737550.1, 510945, 511160, +, 216, 624277; cds-WP_004149864.1, NZ_KK737550.1, 511272, 513017, +, 1746, 1963987; cds-WP_004174339.1, NZ_KK737550.1, 513236, 515077, +, 1842, 6340082; cds-WP_002917636.1, NZ_KK737550.1, 515177, 515683, -, 507, 17658; cds-WP_004174338.1, NZ_KK737550.1, 516020, 516238, -, 219, 32708; cds-WP_032430361.1, NZ_KK737550.1, 516309, 517406, -, 1098, 156284; cds-WP_004185683.1, NZ_KK737550.1, 517403, 517888, -, 486, 10106; cds-AF52_RS01195, NZ_KK737550.1, 517885, 519153, -, 1269, 178415; cds-WP_020806188.1-6, NZ_KK737550.1, 519222, 520202, +, 981, 266570; cds-WP_050484295.1, NZ_KK737550.1, 520559, 522556, -, 1998, 8901350; cds-WP_032430362.1, NZ_KK737550.1, 522572, 523600, -, 1029, 4587138; cds-WP_032414744.1, NZ_KK737550.1, 523929, 524162, -, 234, 1277010; cds-WP_032414845.1, NZ_KK737550.1, 524174, 524362, -, 189, 570705; cds-WP_032430364.1, NZ_KK737550.1, 524546, 526954, -, 2409, 507; cds-AF52_RS27605, NZ_KK737550.1, 527242, 527414, +, 173, 5614; cds-AF52_RS0124695, NZ_KK737550.1, 528005, 528295, +, 291, 16114; cds-WP_021441030.1, NZ_KK737550.1, 528625, 530883, +, 2259, 2317; cds-WP_004177844.1, NZ_KK737550.1, 531147, 531515, -, 369, 0; cds-WP_004177843.1, NZ_KK737550.1, 531549, 533309, -, 1761, 315; cds-WP_004146634.1, NZ_KK737550.1, 533683, 533913, -, 231, 410; cds-WP_013815099.1-76, NZ_KK737550.1, 533996, 534964, -, 969, 75173; cds-WP_023285711.1, NZ_KK737550.1, 535001, 537424, +, 2424, 156421; cds-WP_013815099.1-77, NZ_KK737550.1, 537590, 538558, -, 969, 89402; cds-WP_004198387.1, NZ_KK737550.1, 538641, 539402, -, 762, 3042; cds-WP_002885046.1, NZ_KK737550.1, 539413, 540426, -, 1014, 3153; cds-WP_004151742.1, NZ_KK737550.1, 540423, 541181, -, 759, 7432; cds-WP_032430384.1, NZ_KK737550.1, 541357, 542385, +, 1029, 13984; cds-WP_013815099.1-78, NZ_KK737550.1, 542403, 543371, +, 969, 110986; cds-WP_004174268.1, NZ_KK737550.1, 543545, 543796, -, 252, 41001; cds-WP_032430367.1, NZ_KK737550.1, 543799, 544413, -, 615, 80978; cds-WP_004174272.1, NZ_KK737550.1, 544514, 544750, +, 237, 690110; cds-AF52_RS26555, NZ_KK737550.1, 544785, 545111, +, 327, 1350550; cds-WP_013815099.1-79, NZ_KK737550.1, 545145, 546113, +, 969, 464205; cds-AF52_RS0124710, NZ_KK737550.1, 546172, 546360, +, 189, 33096; cds-WP_004174277.1, NZ_KK737550.1, 546368, 546568, +, 201, 74727; cds-AF52_RS01075, NZ_KK737550.1, 546532, 546735, +, 204, 6457; cds-WP_002885077.1, NZ_KK737551.1, 251, 709, -, 459, 10976; cds-WP_004151738.1, NZ_KK737551.1, 794, 1123, +, 330, 314006; cds-WP_023283136.1, NZ_KK737551.1, 1108, 2712, -, 1605, 401138; cds-WP_004177826.1, NZ_KK737551.1, 3219, 3500, +, 282, 1540; cds-WP_004186517.1, NZ_KK737551.1, 3547, 4404, -, 858, 344; cds-WP_004177833.1, NZ_KK737551.1, 4401, 5234, -, 834, 22813; cds-WP_004177835.1, NZ_KK737551.1, 5294, 6433, -, 1140, 9494; cds-WP_013815099.1-80, NZ_KK737551.1, 6546, 7514, -, 969, 75235; cds-AF52_RS01030, NZ_KK737551.1, 7646, 8203, -, 558, 1716; cds-AF52_RS01025, NZ_KK737551.1, 8492, 9028, -, 537, 140; cds-AF52_RS0124720, NZ_KK737551.1, 9021, 9488, -, 468, 34467; cds-AF52_RS01020, NZ_KK737551.1, 10314, 10625, -, 312, 2529; cds-AF52_RS26200, NZ_KK737551.1, 10743, 10920, +, 178, 10080; cds-AF52_RS01015, NZ_KK737551.1, 11477, 12304, -, 828, 86; cds-AF52_RS01010, NZ_KK737551.1, 12683, 13012, +, 330, 15692; cds-AF52_RS27655, NZ_KK737551.1, 13210, 13361, +, 152, 32413; cds-AF52_RS01000, NZ_KK737551.1, 13462, 13718, +, 257, 45400; cds-WP_002885500.1, NZ_KK737551.1, 14025, 15053, -, 1029, 118784; cds-WP_002885506.1, NZ_KK737551.1, 15095, 15661, +, 567, 1215422; cds-WP_002885511.1, NZ_KK737551.1, 15735, 15866, +, 132, 149218; cds-WP_002885518.1, NZ_KK737551.1, 16026, 16172, +, 147, 69614; cds-WP_002885521.1, NZ_KK737551.1, 16316, 16633, +, 318, 29341; cds-WP_002885523.1, NZ_KK737551.1, 16630, 17163, -, 534, 8214; cds-WP_002885526.1, NZ_KK737551.1, 17277, 17636, -, 360, 20614; cds-WP_002885530.1, NZ_KK737551.1, 17647, 18042, -, 396, 148830; cds-WP_004177729.1, NZ_KK737551.1, 18053, 18787, -, 735, 642493; cds-WP_004177727.1, NZ_KK737551.1, 18780, 20570, -, 1791, 290660; cds-WP_004146714.1, NZ_KK737551.1, 20846, 21823, +, 978, 134808; cds-WP_021441059.1, NZ_KK737551.1, 22006, 23508, +, 1503, 47361; cds-WP_004146716.1, NZ_KK737551.1, 23546, 26875, -, 3330, 621773; cds-WP_002885536.1, NZ_KK737551.1, 26899, 27861, -, 963, 556021; cds-WP_002885537.1, NZ_KK737551.1, 28022, 29083, -, 1062, 496037; cds-WP_002885538.1, NZ_KK737551.1, 29178, 29723, +, 546, 151327; cds-WP_032430397.1, NZ_KK737551.1, 29767, 30498, -, 732, 36883; cds-WP_004210690.1, NZ_KK737551.1, 31431, 32570, -, 1140, 330646; cds-WP_187079197.1, NZ_KK737551.1, 32584, 34095, +, 1512, 26689; cds-WP_004220675.1, NZ_KK737551.1, 34100, 34549, +, 450, 122414; cds-WP_004152021.1, NZ_KK737551.1, 34566, 35921, +, 1356, 143371; cds-WP_004152020.1, NZ_KK737551.1, 35932, 37791, +, 1860, 192600; cds-WP_032430401.1, NZ_KK737551.1, 37784, 38734, +, 951, 2581981; cds-WP_002885659.1, NZ_KK737551.1, 38827, 39135, +, 309, 2354660; cds-WP_004146725.1, NZ_KK737551.1, 39211, 40491, +, 1281, 2492143; cds-WP_004146726.1, NZ_KK737551.1, 40557, 41819, +, 1263, 1863617; cds-WP_002885662.1, NZ_KK737551.1, 41822, 42826, +, 1005, 1456846; cds-WP_002885663.1, NZ_KK737551.1, 42909, 43199, -, 291, 1281; cds-WP_002885664.1, NZ_KK737551.1, 43422, 43619, +, 198, 2929; cds-WP_002885665.1, NZ_KK737551.1, 43722, 45020, +, 1299, 1358278; cds-WP_002885667.1, NZ_KK737551.1, 45171, 45596, +, 426, 251126; cds-WP_004192475.1,
NZ_KK737551.1, 45633, 48065, +, 2433, 1484782; cds-WP_002885669.1, NZ_KK737551.1, 48147, 48878, +, 732, 220617; cds-WP _021441063.1, NZ_KK737551.1, 49004, 50641, +, 1638,141519; cds-WP_002885673.1, NZ_KK737551.1, 50632, 50907, -, 276, 394395; cds-WP_002885674.1, NZ_KK737551.1, 51045, 51353, -, 309, 169439; cds-WP _032428483.1, NZ_KK737551.1, 51563, 52279, +, 717, 135572; cds-WP _004177697.1, NZ_KK737551.1, 52276, 53031, -, 756, 126964; cds-WP _032430402.1, NZ_KK737551.1, 53139, 54203, -, 1065, 70730; cds-WP_016946098.1, NZ_KK737551.1, 54566, 55966, +, 1401, 30815; cds-WP_002885682.1, NZ_KK737551.1, 55979, 56284, +, 306, 8929; cds-WP _002885683.1, NZ_KK737551.1, 56294, 56761, +, 468, 139; cds-WP _002885684.1, NZ_KK737551.1, 56775, 57425, +, 651, 382; cds-WP _004210671.1, NZ_KK737551.1, 57435, 58289, +, 855, 3632; cds-WP_004178347.1, NZ_KK737551.1, 58289, 58975, +, 687, 83; cds-WP_009309325.1, NZ_KK737551.1, 59038, 60231, -, 1194, 3791; cds-WP_002885689.1, NZ_KK737551.1, 60381, 60656, -, 276, 13908; cds-WP_002885691.1, NZ_KK737551.1, 60997, 61392, +, 396, 7072507; cds-WP_002885722.1, NZ_KK737551.1, 61398, 61712, +, 315, 8526092; cds-WP_000135199.1, NZ_KK737551.1, 61717, 61944, +, 228, 8257795; cds-WP _002886682.1, NZ_KK737551.1, 61986, 62435, +, 450, 4461235; cds-WP _004177687.1, NZ_KK737551.1, 62544, 63218, -, 675, 221274; cds-WP _002886687.1, NZ_KK737551.1, 63437, 64057, +, 621, 277090; cds-WP _002886688.1, NZ_KK737551.1, 64362, 65765, +, 1404, 457123; cds-WP _004210666.1, NZ_KK737551.1, 65870, 66532, -, 663, 1158; cds-WP _002886691.1, NZ_KK737551.1, 66615, 67586, -, 972, 0; cds-WP _032430405.1, NZ_KK737551.1, 67662, 68486, -, 825, 29200; cds-WP _021441067.1, NZ_KK737551.1, 68564, 69412, -, 849, 121295; cds-WP _004152015.1, NZ_KK737551.1, 69502, 69873, +, 372, 102132; cds-WP _021441068.1, NZ_KK737551.1, 69928, 71871, -, 1944, 147336; cds-WP _032430406.1, NZ_KK737551.1, 72063, 72806, +, 744, 58459; cds-WP _002886706.1, NZ_KK737551.1, 72796, 73353, -, 558, 58536; cds-WP _002886707.1, NZ_KK737551.1, 73677, 73883, +, 207, 96597; cds-WP _004178353.1, NZ_KK737551.1, 73945, 74817, -, 873, 267826; cds-WP_004222103.1, NZ_KK737551.1, 74848, 76857, -, 2010, 79724; cds-WP _016946104.1, NZ_KK737551.1, 76876, 78108, -, 1233, 11961; cds-WP _002886718.1, NZ_KK737551.1, 78310, 79647, -, 1338, 316084; cds-WP _002886721.1, NZ_KK737551.1, 79828, 80466, -, 639, 202284; cds-WP _002886722.1, NZ_KK737551.1, 80675, 82408, +, 1734, 523771; cds-WP_032430407.1, NZ_KK737551.1, 82405, 86181, +, 3777, 414325; cds-WP _002886724.1, NZ_KK737551.1, 86184, 86528, +, 345, 179276; cds-WP_021441069.1, NZ_KK737551.1, 86566, 87039, -, 474, 7465; cds-WP_171789277.1, NZ_KK737551.1, 87011, 87733, -, 723, 133618; cds-WP _021441070.1, NZ_KK737551.1, 87889, 89076, -, 1188, 251445; cds-WP_002886766.1, NZ_KK737551.1, 89151, 89681, -, 531, 1637604; cds-WP_002886769.1, NZ_KK737551.1, 90085, 91041, +, 957, 231209; cds-AF52_RS00620, NZ_KK737551.1, 91165, 92456, +, 1292, 87819; cds-WP_032430409.1, NZ_KK737551.1, 92467, 93492, +, 1026, 771; cds-WP_021312675.1, NZ_KK737551.1, 93479, 94477, +, 999, 163629; cds-WP _002886827.1, NZ_KK737551.1, 94520, 95518, -, 999, 3494421; cds-WP _002886886.1, NZ_KK737551.1, 95694, 97067, +, 1374, 439488; cds-WP_004177666.1, NZ_KK737551.1, 97136, 97663, +, 528, 53707; cds-WP _032430410.1, NZ_KK737551.1, 97707, 98915, -, 1209, 3935; cds-WP _004177664.1, NZ_KK737551.1, 99407, 99958, -, 552, 310983; cds-WP _004152265.1, NZ _KK737551.1, 100054, 101406, +, 1353, 128773; cds-WP _021441074.1, NZ_KK737551.1, 101443, 101829, +, 387, 405498; cds-WP_002886894.1, NZ_KK737551.1, 102119, 102457, +, 339, 30976; cds-WP_002886895.1, NZ_KK737551.1, 102468, 102830, +, 363, 54054; cds-WP _002886896.1, NZ _KK737551.1, 102833, 103132, +, 300, 26800; cds-WP _002886897.1, NZ_KK737551.1, 103154, 103930, +, 777, 117859; cds-WP _004152267.1, NZ_KK737551.1, 103944, 104588, +, 645, 80725; cds-WP _004152268.1, NZ_KK737551.1, 104694, 105827, +, 1134, 136190; cds-WP _021441075.1, NZ_KK737551.1, 105811, 106929, +, 1119, 101248; cds-WP_002886899.1, NZ_KK737551.1, 106926, 107666, +, 741, 42288; cds-WP _002886900.1, NZ_KK737551.1, 107733, 108887, +, 1155, 8249; cds-WP _004152270.1, NZ _KK737551.1, 108910, 110820, +, 1911, 7485; cds-WP _032430412.1, NZ_KK737551.1, 110898, 111140, +, 243, 73467; cds-WP_002886902.1, NZ _KK737551.1, 111130, 111414, +, 285, 106253; cds-WP _004222151.1, NZ_KK737551.1, 111418, 111882, -, 465, 192640; cds-WP _004186701.1, NZ_KK737551.1, 112190, 114328, -, 2139, 92496; cds-WP_021441079.1, NZ_KK737551.1, 114685, 115428, -, 744, 28845; cds-WP_032410138.1, NZ _KK737551.1, 115431, 115604, -, 174, 2792; cds-WP_004178377.1, NZ_KK737551.1, 115689, 115997, +, 309, 18; cds-WP _021441081.1, NZ_KK737551.1, 116088, 117395, +, 1308, 11293; cds-WP_004146783.1, NZ_KK737551.1, 117422, 118774, +, 1353, 534131; cds-WP_004178379.1, NZ_KK737551.1, 118830, 120485, -, 1656, 2889100; cds-WP_021441082.1, NZ_KK737551.1, 120537, 121955, -, 1419, 5970343; cds-WP _004177658.1, NZ_KK737551.1, 122086, 123033, -, 948, 11346; cds-WP_009309303.1, NZ_KK737551.1, 123413, 126121, +, 2709, 273244; cds-WP_002886926.1, NZ_KK737551.1, 126195, 126581, -, 387, 228563; cds-WP _002886927.1, NZ_KK737551.1, 126734, 127195, -, 462, 377161; cds-WP_002886928.1, NZ_KK737551.1, 127209, 128144, -, 936, 944629; cds-WP_002886930.1, NZ_KK737551.1, 128181, 128285, -, 105, 2095; cds-WP _002886939.1, NZ_KK737551.1, 128480, 128932, +, 453, 0; cds-WP _002886940.1, NZ_KK737551.1, 129175, 130149, +, 975, 60723; cds-WP_002886941.1, NZ_KK737551.1, 130161, 130991, +, 831, 34085; cds-WP _021441084.1, NZ _KK737551.1, 131086, 132651, +, 1566, 38909; cds-WP_004152274.1, NZ_KK737551.1, 132708, 134126, +, 1419, 15773; cds-WP_002886945.1, NZ_KK737551.1, 134211, 134936, +, 726, 215773; cds-WP _002886946.1, NZ_KK737551.1, 134939, 135943, -, 1005, 60827; cds-WP_002886947.1, NZ_KK737551.1, 136107, 136529, +, 423, 484177; cds-WP_004186726.1, NZ_KK737551.1, 136588, 137352, +, 765, 940910; cds-WP _004152275.1, NZ_KK737551.1, 137625, 138128, -, 504, 84682; cds-WP_032430414.1, NZ_KK737551.1, 138249, 141104, -, 2856, 3194044; cds-WP_002886953.1, NZ_KK737551.1, 141104, 141547, -, 444, 395535; cds-WP_002886954.1, NZ_KK737551.1, 141667, 143178, -, 1512, 1354534; cds-WP _002886955.1, NZ_KK737551.1, 143568, 144665, +, 1098, 286102; cds-WP_002886956.1, NZ_KK737551.1, 144665, 145747, +, 1083, 171654; cds-WP _002886957.1, NZ_KK737551.1, 145796, 147298, -, 1503, 335237; cds-WP_032430415.1, NZ_KK737551.1, 147397, 148218, -, 822, 70371; cds-WP _004177655.1, NZ_KK737551.1, 148569, 150074, +, 1506, 663347; cds-WP_004186731.1, NZ_KK737551.1, 150093, 150902, +, 810, 758337; cds-WP_004152190.1, NZ_KK737551.1, 151887, 152744, -, 858, 235889; cds-WP _004146797.1, NZ_KK737551.1, 152896, 154809, -, 1914, 1196251; cds-WP_004186736.1, NZ_KK737551.1, 155315, 157255, +, 1941, 596595; cds-WP _004186740.1, NZ_KK737551.1, 157302, 158315, +, 1014, 1275171; cds-WP_021314296.1, NZ_KK737551.1, 158357, 159241, +, 885, 1047858; cds-WP_002886970.1, NZ_KK737551.1, 159265, 160164, +, 900, 273861; cds-WP_021441086.1, NZ_KK737551.1, 160308, 161618, -, 1311, 41062; cds-WP_021441087.1, NZ_KK737551.1, 161758, 162828, -, 1071, 41617; cds-WP _032430419.1, NZ_KK737551.1, 162834, 163658, -, 825, 0; cds-WP _004177643.1, NZ_KK737551.1, 163669, 164556, -, 888, 0; cds-WP_012737244.1, NZ_KK737551.1, 164546, 165430, -, 885, 1894; cds-WP _004178400.1, NZ_KK737551.1, 165561, 166580, -, 1020, 65234; cds-WP_021441090.1, NZ_KK737551.1, 166692, 168161, -, 1470, 22718; cds-WP _032428490.1, NZ _KK737551.1, 168158, 168832, -, 675, 309; cds-AF52_RS00275, NZ _KK737551.1, 169708, 170763, +, 1056, 82265; cds-WP _013815099.1-81, NZ_KK737551.1, 170797, 171765, +, 969, 94175; cds-WP _002887608.1, NZ_KK737551.1, 171853, 172632, +, 780, 25172; cds-WP_032430421.1, NZ_KK737551.1, 172610, 173461, +, 852, 59149; cds-WP_032428518.1, NZ_KK737551.1, 173474, 174535, +, 1062, 484; cds-WP_004146946.1, NZ_KK737551.1, 174553, 175563, +, 1011, 16519; cds-WP _021441152.1, NZ_KK737551.1, 175697, 176629, +, 933, 8340; cds-WP_002887618.1, NZ_KK737551.1, 176684, 178975, -, 2292, 920815; cds-WP_002887620.1, NZ_KK737551.1, 179233, 179724, -, 492, 52692; cds-WP _002887623.1, NZ_KK737551.1, 179790, 180527, -, 738, 182642; cds-WP _004178536.1, NZ_KK737551.1, 180530, 181069, -, 540, 10626; cds-WP_002887631.1, NZ_KK737551.1, 181172, 181645, -, 474, 291; cds-WP_004146059.1, NZ_KK737551.1, 181636, 182412, -, 777, 3916; cds-WP_032430423.1, NZ_KK737551.1, 182692, 183876, +, 1185, 756415; cds-WP _009485342.1, NZ_KK737551.1, 183893, 184774, +, 882, 147998; cds-WP _002887645.1, NZ_KK737551.1, 185040, 185762, +, 723, 146; cds-WP_002887647.1, NZ_KK737551.1, 185723, 186397, +, 675, 89322; cds-WP_004151383.1, NZ_KK737551.1, 186403, 186861, -, 459, 48640; cds-AF52_RS0124785, NZ _KK737551.1, 187200, 187601, +, 402, 19689; cds-WP _013815099.1-82, NZ _KK737551.1, 187633, 188601, +, 969, 69479; cds-AF52_RS00180, NZ_KK737551.1, 188659, 189609, +, 951, 1916; cds-WP _002887652.1, NZ_KK737551.1, 189659, 190447, -, 789, 288847; cds-AF52_RS27680, NZ_KK737551.1, 190554, 190682, -, 129, 1526; cds-WP_013815099.1-83, NZ_KK737551.1, 190741, 191709, -, 969, 92087; cds-AF52_RS00165, NZ _KK737551.1, 191743, 192666, -, 924, 10475; cds-WP_004221147.1, NZ _KK737551.1, 192690, 192860, -, 171, 10705; cds-WP _002887654.1, NZ_KK737551.1, 192931, 193170, +, 240, 117562; cds-WP _023317511.1, NZ_KK737551.1, 193450, 194478, -, 1029, 257008; cds-WP_015959390.1, NZ_KK737551.1, 194581, 194994, +, 414, 25584; cds-WP_021441160.1, NZ_KK737551.1, 194963, 195409, +, 447, 127968; cds-WP _004146044.1, NZ_KK737551.1, 195424, 196101, +, 678, 253479; cds-WP_002887711.1, NZ_KK737551.1, 196192, 197781, +, 1590, 1118682; cds-WP _032430427.1, NZ_KK737551.1, 198001, 198621, +, 621, 101878; cds-WP_002887716.1, NZ_KK737551.1, 198751, 198912, +, 162, 65182; cds-WP _021441162.1, NZ_KK737551.1, 199066, 200139, +, 1074, 56985; cds-WP_004177496.1, NZ_KK737551.1, 200136, 200930, +, 795, 12689; cds-WP_004146034.1, NZ_KK737551.1, 201061, 202338, +, 1278, 456165; cds-WP _004146033.1, NZ_KK737551.1, 202734, 203534, +, 801, 280957; cds-WP _004178548.1, NZ_KK737551.1, 203648, 204970, +, 1323, 221140; cds-WP _004151380.1, NZ_KK737551.1, 205023, 206246, +, 1224, 751457; cds-WP_002887789.1, NZ_KK737551.1, 206349, 207068, +, 720, 689062; cds-WP_032430428.1, NZ_KK737551.1, 207148, 208164, -, 1017, 152621; cds-WP_002887794.1, NZ_KK737551.1, 208164, 208811, -, 648, 75108; cds-WP_004177487.1, NZ_KK737551.1, 208916, 209887, +, 972, 173810; cds-WP _002887800.1, NZ_KK737551.1, 209897, 211279, +, 1383, 156119; cds-WP_002887802.1, NZ_KK737551.1, 211301, 212533, +, 1233, 257422; cds-WP _002887805.1, NZ_KK737551.1, 212627, 214294, -, 1668, 2666600; cds-WP _002887806.1, NZ_KK737551.1, 214523, 216460, +, 1938, 294289; cds-WP _002887809.1, NZ_KK737551.1, 216549, 216878, +, 330, 37604; cds-WP_002887810.1, NZ_KK737551.1, 216865, 217380, -, 516, 116598; cds-WP_002887811.1, NZ_KK737551.1, 217494, 218141, +, 648, 220817; cds-WP _002887812.1, NZ_KK737551.1, 218138, 219007, -, 870, 760718; cds-WP_002887814.1, NZ_KK737551.1, 219217, 219690, +, 474, 149466; cds-WP_002887820.1, NZ_KK737551.1, 219703, 220392, +, 690, 93493; cds-WP _021441167.1, NZ_KK737551.1, 220392, 221816, +, 1425, 85619; cds-WP _004214415.1, NZ_KK737551.1, 221877, 223235, +, 1359, 20858; cds-WP_002887843.1, NZ_KK737551.1, 223298, 224014, -, 717, 3228798; cds-WP_004146984.1, NZ_KK737551.1, 224110, 224250, +, 141, 573050; cds-WP _002887846.1, NZ_KK737551.1, 224647, 225333, +, 687, 326759;

TABLE 11

RNA FISH probes

| | | | | |
|---|---|---|---|---|
| Name | Species | Gene | Sequence (5′-3′) | Fluorophore |
| EUB338 | Most bacteria | 16s rRNA | GCT GCC TCC CGT AGG AGT (SEQ ID NO: 778) | Cy3 |
| cspD51 | MGH66 | cspD | TCGCCGCCGCCCTCAGGGCA (SEQ ID NO: 779) | Alexa488 |
| cspD53 | MGH67 | cspD | CGCCGCCGCCCTCAGGGCAGA (SEQ ID NO: 780) | Alexa488 |
| cspD55 | MGH68 | cspD | TCGCCGCCGCCCTCAGGGCA (SEQ ID NO: 781) | Alexa488 |
| rihC173 | MGH66 | rihC | GCGCCCTGCGCCAGCGGTACATCGG (SEQ ID NO: 782) | Cy3 |
| rihC431 | MGH66 | rihC | GCCGCCCATGATCACCAGCCGGC (SEQ ID NO: 783) | Cy3 |
| rihC559 | MGH66 | rihC | GCCCGGTTGGTGACGTCCAGCCC (SEQ ID NO: 784) | Cy3 |
| rihC715 | MGH66 | rihC | GTTCCGGGCGGGCCAGCCACGC (SEQ ID NO: 785) | Cy3 |
| rihC817 | MGH66 | rihC | CGCCGGCTGGCCCAGTCGTCC (SEQ ID NO: 786) | Cy3 |
| rseB224 | MGH66 | rseB | TCCCGGCGCGGACCGTCCAGCT (SEQ ID NO: 787) | Alexa488 |
| rseB520 | MGH66 | rseB | CTCCAGCGTTTCCCCGTCGCGGTC (SEQ ID NO: 788) | Alexa488 |
| rseB675 | MGH66 | rseB | CAGTCACGCCCTGCGGGAGCCACGTC (SEQ ID NO: 789) | Alexa488 |
| yidC145 | MGH66 | yidC | CTGGCCACTGGCCGGTACGCCCTG (SEQ ID NO: 790) | Cy3 |
| yidC323 | MGH66 | yidC | GGCCGGTTAAGCCGCTCTGCGCCT (SEQ ID NO: 791) | Cy3 |
| yidC872 | MGH66 | yidC | GGCCTGGCTGCACCAGTACCGGCT (SEQ ID NO: 792) | Cy3 |
| yidC914 | MGH66 | yidC | GCCGGGCCGACCCACAGCGTGC (SEQ ID NO: 793) | Cy3 |

TABLE 12

Summary of genera captured by 16s rRNA DNA sequencing and BacDrop

| | Number of genera |
|---|---|
| Total genera captured | 58 (16s rRNA); 127 (BacDrop) |
| Genera captured by both methods | 54 |
| Genera captured only by 16s rRNA | 54 |
| Genera captured only by BacDrop | 73 |

TABLE 13

Genera captured

| Genus | 16s rRNA | BacDrop |
|---|---|---|
| Lachnoclostridium | 4.156 | 4.295 |
| Roseburia | 0.726 | 0.606 |
| Blautia | 0.356 | 2.263 |
| Anaerostipes | 0.109 | 1.358 |
| Cellulosilyticum | 0.014 | 0.281 |
| Butyrivibrio | 0.002 | 1.361 |
| Eubacterium | 6.977 | 1.444 |
| Clostridium | 2.010 | 1.895 |
| Candidatus Arthromitus | 0.141 | 0.013 |
| Ruminococcus | 0.297 | 0.370 |
| Oscillibacter | 1.458 | 0.975 |
| Dehalobacter | 0.007 | 0.023 |
| Mogibacterium | 0.117 | 0.088 |
| Halanaerobium | 0.002 | 0.008 |
| Lactobacillus | 5.848 | 6.242 |
| Streptococcus | 0.014 | 0.080 |
| Lactococcus | 0.005 | 0.007 |
| Leuconostoc | 0.002 | 0.001 |
| Bacillus | 0.011 | 1.091 |
| Bacillales incertae sedis><wbr>Bacillales Family XI. Incertae Sedis><wbr>Gemella | 0.007 | 0.019 |
| Staphylococcaceae><wbr>Staphylococcus | 0.004 | 0.271 |
| Gordonibacter | 0.009 | 0.007 |
| Olsenella | 0.071 | 0.011 |
| Corynebacteriaceae><wbr>Corynebacterium | 0.032 | 0.127 |
| Bifidobacteriales><wbr>Bifidobacteriaceae><wbr>Bifidobacterium | 0.016 | 0.035 |
| Streptomyces | 0.012 | 0.020 |
| Mycoplasma | 0.283 | 0.069 |
| Ureaplasma | 0.224 | 0.015 |
| Spiroplasmataceae><wbr>Spiroplasma | 0.039 | 0.007 |
| Acholeplasmatales><wbr>Acholeplasmataceae><wbr>Candidatus Phytoplasma | 0.039 | 0.009 |
| Chloroflexi><wbr>...><wbr>Dehalococcoides | 0.073 | 0.003 |
| Chloroflexi><wbr>Dehalococcoidia><wbr>Dehalogenimonas | 0.005 | NA |
| Deinococcus-Thermus><wbr>...><wbr>Thermus | 0.002 | 0.001 |
| Muribaculum | 6.980 | 1.591 |
| Prevotella | 4.265 | 1.706 |
| Alistipes | 2.828 | 0.151 |
| Bacteroidaceae><wbr>Bacteroides | 1.787 | 2.525 |
| Parabacteroides | 0.402 | 0.764 |
| Tannerella | 0.037 | 0.102 |
| Porphyromonas | 0.004 | 0.295 |
| Runella | 0.279 | 0.020 |
| Spirosoma | 0.002 | 0.013 |
| Cellulophaga | 0.012 | 0.004 |
| Gramella | 0.002 | NA |
| Blattabacteriaceae><wbr>Blattabacterium | 0.002 | 0.004 |
| Gemmatimonas | 0.052 | 0.004 |
| Helicobacter | 7.076 | 1.040 |
| Campylobacteraceae><wbr>Campylobacter | 0.002 | 0.023 |
| Desulfovibrio | 0.521 | 0.937 |
| Klebsiella | 0.002 | 0.011 |
| Psychrobacter | 0.005 | 0.001 |
| Acinetobacter | 0.002 | 0.007 |
| Pseudomonadales><wbr>Pseudomonadaceae><wbr>Pseudomonas | 0.004 | 0.019 |
| Pasteurellales><wbr>Pasteurellaceae><wbr>Aggregatibacter | 0.002 | NA |
| Nitrosomonadaceae><wbr>Nitrosomonas | 0.002 | NA |
| Verrucomicrobiae><wbr>...><wbr>Akkermansia | 0.043 | 0.042 |
| Chlamydiae><wbr>...><wbr>Chlamydia | 0.002 | 0.004 |
| Nitrospirae><wbr>...><wbr>Leptospirillum | 0.007 | 0.001 |
| Acetobacterium | NA | 0.016 |
| Desulfotomaculum | NA | 0.232 |
| Desulfitobacterium | NA | 0.092 |
| Desulfosporosinus | NA | 0.009 |
| Clostridiales incertae sedis><wbr>Clostridiales Family XVII. Incertae Sedis><wbr> Thermaerobacter | NA | 0.037 |
| Thermoanaerobacter | NA | 0.011 |
| Thermoanaerobacterales Family III. Incertae Sedis><wbr>Thermoanaerobacterium | NA | 0.008 |
| Thermoanaerobacterales Family III. Incertae Sedis><wbr>Caldicellulosiruptor | NA | 0.005 |
| Enterococcaceae><wbr>Enterococcus | NA | 0.068 |
| Oenococcus | NA | 0.004 |
| Geobacillus | NA | 0.024 |
| Lysinibacillus | NA | 0.011 |
| Paenibacillus | NA | 0.178 |
| Bacillales incertae sedis><wbr>Bacillales Family XII. Incertae Sedis><wbr>Exiguobacterium | NA | 0.015 |
| Dialister | NA | 0.035 |
| Megasphaera | NA | 0.029 |
| Selenomonas | NA | 0.049 |
| Sporomusaceae><wbr>Pelosinus | NA | 0.009 |
| Acidaminococcales><wbr>Acidaminococcaceae><wbr>Phascolarctobacterium | NA | 0.029 |
| Anaerococcus | NA | 0.028 |
| Leucobacter | NA | 0.001 |
| Arthrobacter | NA | 0.004 |
| Microlunatus | NA | 0.016 |
| Nocardioidaceae><wbr>Aeromicrobium | NA | 0.013 |
| Streptosporangiales><wbr>Thermomonosporaceae><wbr>Actinomadura | NA | 0.001 |
| Chloroflexi><wbr>...><wbr>Chloroflexus | NA | 0.007 |
| Tenacibaculum | NA | 0.005 |
| Antarcticibacterium | NA | 0.005 |
| Flavobacterium | NA | 0.004 |
| Hymenobacter | NA | 0.023 |
| Sphingobacterium | NA | 0.008 |
| Mucilaginibacter | NA | 0.003 |
| Chitinophaga | NA | 0.007 |
| Arachidicoccus | NA | 0.004 |
| Pseudodesulfovibrio | NA | 0.012 |
| Desulfomicrobiaceae><wbr>Desulfomicrobium | NA | 0.015 |
| Desulfobacterales><wbr>Desulfobacteraceae><wbr>Desulfobacter | NA | 0.001 |
| Geobacteraceae><wbr>Geobacter | NA | 0.013 |
| Myxococcaceae><wbr>Myxococcus | NA | 0.004 |
| Anaeromyxobacteraceae><wbr>Anaeromyxobacter | NA | 0.001 |
| Campylobacteraceae><wbr>Arcobacter | NA | 0.020 |
| Kosakonia | NA | 0.003 |
| Pectobacteriaceae><wbr>Dickeya | NA | 0.004 |
| Erwiniaceae><wbr>Pantoea | NA | 0.008 |
| Yersiniaceae><wbr>Serratia | NA | 0.007 |
| Legionellales><wbr>Legionellaceae><wbr>Legionella | NA | 0.007 |
| Vibrio | NA | 0.025 |
| Moraxella | NA | 0.003 |
| Pasteurellales><wbr>Pasteurellaceae><wbr>Haemophilus | NA | 0.013 |
| Alteromonadales><wbr>Colwelliaceae><wbr>Colwellia | NA | 0.003 |
| Thiotrichales><wbr>Francisellaceae><wbr>Francisella | NA | 0.004 |
| unclassified Rhizobiales><wbr>Methyloceanibacter | NA | 0.001 |
| Rhizobiaceae><wbr>Sinorhizobium/Ensifer group><wbr>Sinorhizobium | NA | 0.003 |
| Methylocystaceae><wbr>Methylocystis | NA | 0.001 |
| Paracoccus | NA | 0.004 |
| Labrenzia | NA | 0.001 |
| Rhodospirillales><wbr>Rhodospirillaceae><wbr>Azospirillum | NA | 0.003 |
| Roseomonas | NA | 0.001 |
| Neorickettsia | NA | 0.004 |
| Wolbachieae><wbr>Wolbachia | NA | 0.003 |
| Anaplasma | NA | 0.001 |
| unclassified Burkholderiales><wbr>Burkholderiales Genera incertae sedis><wbr>Thiomonas | NA | 0.003 |
| Alcaligenaceae><wbr>Bordetella | NA | 0.005 |
| Neisseriaceae><wbr>Neisseria | NA | 0.007 |
| Oligoflexia><wbr>...><wbr>Bdellovibrio | NA | 0.003 |
| Planctomycetia><wbr>...><wbr>Planctopirus | NA | 0.001 |
| Sphaerochaeta | NA | 0.025 |
| Treponema | NA | 0.015 |
| Spirochaetes><wbr>...><wbr>Brachyspira | NA | 0.003 |
| Acidobacteria><wbr>...><wbr>Terriglobus | NA | 0.005 |
| Thermotogae><wbr>...><wbr>Marinitoga | NA | 0.004 |
| Thermodesulfobacteria><wbr>...><wbr>Thermodesulfobacterium | NA | 0.001 |
| Nitrospirae><wbr>...><wbr>Nitrospira | NA | 0.001 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of generating a single prokaryotic cell cDNA library, wherein the RNAs from different prokaryotic cells are barcoded individually, allowing the cell identity of each RNA to be retained in a single library, the method comprising:
a. degrading ribosomal RNA (rRNA) and genomic DNA (gDNA) in a set of fixed and permeabilized cells;
b. labeling each mRNA in the fixed and permeabilized cells, the labeling comprising separating the fixed and permeabilized cells into a plurality of first reaction volumes, reverse transcribing the mRNA into cDNA in the fixed and permeabilized cells, and adding a first barcode, a unique molecular identifier (UMI), and a poly-nucleotide tail to each first strand cDNA;
c. labeling each cDNA from a same cell in the fixed and permeabilized cells, the labeling comprising separating the fixed and permeabilized cells into separate individual discrete second reaction volumes, conducting a second strand cDNA synthesis in the fixed and permeabilized cells, and adding a second barcode unique to that reaction volume to each second strand cDNA, thus labeling each cDNA with a cell-specific first and second barcode combination and a UMI; and
d. amplifying the labeled cDNA and preparing a sequencing library comprising the amplified labeled cDNA.

2. The method of claim 1, further comprising, prior to the degrading step, preparing a set of fixed and permeabilized cells, the preparing comprising:
a. fixing each cell of a set of prokaryotic cells; and
b. permeabilizing each fixed cell of the set of prokaryotic cells.

3. The method of claim 1, wherein each first reaction volume comprises a plurality of RT primers, and wherein each RT primer comprises (i) a 5′ primer binding sequence (ii) a barcode sequence that is the same for all primers in the same first reaction volume but differs from the barcode sequence of primers in any other first reaction volume, and (iii) a unique molecular identifier (UMI) sequence that is different for each primer in a first reaction volume.

4. The method of claim 1, wherein each second reaction volume comprises a single bead and a plurality of primers capable of hybridizing to the poly-nucleotide tail, wherein each bead comprises a plurality of capture oligonucleotides attached 5′ to the bead surface, and wherein each capture oligonucleotide comprises (i) a barcode sequence that is the same for all capture oligonucleotides on the same bead but differs from the barcode sequence of capture oligonucleotides on other beads and (ii) a capture sequence that is the same as the 5′ primer binding sequence in each RT primer.

5. The method of claim 1, wherein the generating second strand cDNA step is accomplished by linear amplification using the primers capable of hybridizing to the poly-nucleotide tail.

6. The method of claim 1, wherein the second barcode is a bead barcode sequence, and wherein the adding a second barcode step is accomplished by amplifying the second strand cDNA using the capture sequence on the capture oligonucleotides attached to the bead to prime linear amplification.

7. The method of claim 1, wherein mRNA accounts for more than 5%, more than 10%, more than 20%, more than 30%, more than 40%, more than 50%, more than 60%, more than 70%, or more than 80% of total aligned reads.

8. The method of claim 1, wherein the mean mRNA UMI per cell is more than 25, more than 35, more than 45, more than 55, or more than 65.

9. The method of claim 1, wherein each second reaction volume can be loaded with multiple fixed and permeabilized cells.

10. The method of claim 1, wherein each second reaction volume comprises one, two, three, four, five, or six fixed and permeabilized cells.

11. The method of claim 1, wherein each second reaction volume is an aqueous in oil droplet.

12. The method of claim 1, wherein the sequencing library can distinguish species heterogeneity, strain heterogeneity, genotypic heterogeneity and/or phenotypic heterogeneity of the plurality of prokaryotic cells.

13. The method of claim 1, wherein the sequencing library can distinguish species heterogeneity, strain heterogeneity, genotypic heterogeneity and/or phenotypic heterogeneity of one or more uncharacterized organisms, an environmental microbiome, or an organismal microbiome.

14. The method of claim 1, wherein the sequencing library can distinguish phenotypic heterogeneity in the response of the plurality of prokaryotic cells to one or more drug treatments.

15. The method of claim 1, wherein the sequencing library can distinguish phenotypic heterogeneity in the response of the plurality of prokaryotic cells to one or more antibiotic treatments.

16. The method of claim 1, wherein the set of prokaryotic cells is a biological sample obtained from a subject.
